SEQUENCE LISTING (180 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - RNA, 3819 nt, synthetic construct
  source 1..3819: mol_type = other RNA
atgtttgtgtttcttgtgctgctgcctcttgtgtcttctcagtgtgtgaatttgacaacaagaacacagctgccaccagcttatacaaattcttttaccagaggagtgtattatcctgataaagtgtttagatcttctgtgctgcacagcacacaggacctgtttctgccattttttagcaatgtgacatggtttcatgcaattcatgtgtctggaacaaatggaacaaaaagatttgataatcctgtgctgccttttaatgatggagtgtattttgcttcaacagaaaagtcaaatattattagaggatggatttttggaacaacactggattctaaaacacagtctctgctgattgtgaataatgcaacaaatgtggtgattaaagtgtgtgaatttcagttttgtaatgatccttttctgggagtgtattatcacaaaaataataaatcttggatggaatctgaatttagagtgtattcctctgcaaataattgtacatttgaatatgtgtctcagccttttctgatggatctggaaggaaaacagggcaattttaaaaatctgagagaatttgtgtttaaaaatattgatggatattttaaaatttattctaaacacacaccaattaatttagtgagagatctgcctcagggattttctgctctggaacctctggtggatctgccaattggcattaatattacaagatttcagacactgctggctctgcacagatcttatctgacacctggagattcttcttctggatggacagccggagctgcagcttattatgtgggctatctgcagccaagaacatttctgctgaaatataatgaaaatggaacaattacagatgctgtggattgtgctctggatcctctgtctgaaacaaaatgtacattaaaatcttttacagtggaaaaaggcatttatcagacatctaattttagagtgcagccaacagaatctattgtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaaaatctacaaatttagtgaaaaataaatgtgtgaattttaattttaatggattaacaggaacaggagtgctgacagaatctaataaaaaatttctgccttttcagcagtttggcagagatattgcagataccacagatgcagtgagagatcctcagacattagaaattctggatattacaccttgttcttttgggggtgtgtctgtgattacacctggaacaaatacatctaatcaggtggctgtgctgtatcaggatgtgaattgtacagaagtgccagtggcaattcatgcagatcagctgacaccaacatggagagtgtattctacaggatctaatgtgtttcagacaagagcaggatgtctgattggagcagaacatgtgaataattcttatgaatgtgatattccaattggagcaggcatttgtgcatcttatcagacacagacaaattccccaaggagagcaagatctgtggcatctcagtctattattgcatacaccatgtctctgggagcagaaaattctgtggcatattctaataattctattgctattccaacaaattttaccatttctgtgacaacagaaattttacctgtgtctatgacaaaaacatctgtggattgtaccatgtacatttgtggagattctacagaatgttctaatctgctgctgcagtatggatctttttgtacacagctgaatagagctttaacaggaattgctgtggaacaggataaaaatacacaggaagtgtttgctcaggtgaaacagatttacaaaacaccaccaattaaagattttggaggatttaattttagccagattctgcctgatccttctaaaccttctaaaagatcttttattgaagatctgctgtttaataaagtgacactggcagatgcaggatttattaaacagtatggagattgcctgggtgatattgctgcaagagatctgatttgtgctcagaaatttaatggactgacagtgctgcctcctctgctgacagatgaaatgattgctcagtacacatctgctttactggctggaacaattacaagcggatggacatttggagctggagctgctctgcagattccttttgcaatgcagatggcttacagatttaatggaattggagtgacacagaatgtgttatatgaaaatcagaaactgattgcaaatcagtttaattctgcaattggcaaaattcaggattctctgtcttctacagcttctgctctgggaaaactgcaggatgtggtgaatcagaatgcacaggcactgaatactctggtgaaacagctgtctagcaattttggggcaatttcttctgtgctgaatgatattctgtctagactggataaagtggaagctgaagtgcagattgatagactgatcacaggaagactgcagtctctgcagacttatgtgacacagcagctgattagagctgctgaaattagagcttctgctaatctggctgctacaaaaatgtctgaatgtgtgctgggacagtcaaaaagagtggatttttgtggaaaaggatatcatctgatgtcttttccacagtctgctccacatggagtggtgtttttacatgtgacatatgtgccagcacaggaaaagaattttaccacagcaccagcaatttgtcatgatggaaaagcacattttccaagagaaggagtgtttgtgtctaatggaacacattggtttgtgacacagagaaatttttatgaacctcagattattacaacagataatacatttgtgtcaggaaattgtgatgtggtgattggaattgtgaataatacagtgtatgatccactgcagccagaactggattcttttaaagaagaactggataaatattttaaaaatcacacatctcctgatgtggatttaggagatatttctggaatcaatgcatctgtggtgaatattcagaaagaaattgatagactgaatgaagtggccaaaaatctgaatgaatctctgattgatctgcaggaacttggaaaatatgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacaca

SEQ ID NO: 3 - protein, 218 aa, synthetic construct
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 4 - RNA, 654 nt, synthetic construct
  source 1..654: mol_type = other RNA
atgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaa

SEQ ID NO: 5 - protein, 266 aa, synthetic construct
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 6 - RNA, 798 nt, synthetic construct
  source 1..798: mol_type = other RNA
atgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggccggtccctggaggtgctgttccagggccccggc

SEQ ID NO: 7 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - RNA, 3819 nt, synthetic construct
  source 1..3819: mol_type = other RNA
atgtttgtgtttcttgtgctgctgcctcttgtgtcttctcagtgtgtgaatttgacaacaagaacacagctgccaccagcttatacaaattcttttaccagaggagtgtattatcctgataaagtgtttagatcttctgtgctgcacagcacacaggacctgtttctgccattttttagcaatgtgacatggtttcatgcaattcatgtgtctggaacaaatggaacaaaaagatttgataatcctgtgctgccttttaatgatggagtgtattttgcttcaacagaaaagtcaaatattattagaggatggatttttggaacaacactggattctaaaacacagtctctgctgattgtgaataatgcaacaaatgtggtgattaaagtgtgtgaatttcagttttgtaatgatccttttctgggagtgtattatcacaaaaataataaatcttggatggaatctgaatttagagtgtattcctctgcaaataattgtacatttgaatatgtgtctcagccttttctgatggatctggaaggaaaacagggcaattttaaaaatctgagagaatttgtgtttaaaaatattgatggatattttaaaatttattctaaacacacaccaattaatttagtgagagatctgcctcagggattttctgctctggaacctctggtggatctgccaattggcattaatattacaagatttcagacactgctggctctgcacagatcttatctgacacctggagattcttcttctggatggacagccggagctgcagcttattatgtgggctatctgcagccaagaacatttctgctgaaatataatgaaaatggaacaattacagatgctgtggattgtgctctggatcctctgtctgaaacaaaatgtacattaaaatcttttacagtggaaaaaggcatttatcagacatctaattttagagtgcagccaacagaatctattgtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaaaatctacaaatttagtgaaaaataaatgtgtgaattttaattttaatggattaacaggaacaggagtgctgacagaatctaataaaaaatttctgccttttcagcagtttggcagagatattgcagataccacagatgcagtgagagatcctcagacattagaaattctggatattacaccttgttcttttgggggtgtgtctgtgattacacctggaacaaatacatctaatcaggtggctgtgctgtatcaggatgtgaattgtacagaagtgccagtggcaattcatgcagatcagctgacaccaacatggagagtgtattctacaggatctaatgtgtttcagacaagagcaggatgtctgattggagcagaacatgtgaataattcttatgaatgtgatattccaattggagcaggcatttgtgcatcttatcagacacagacaaattccccaaggagagcaagatctgtggcatctcagtctattattgcatacaccatgtctctgggagcagaaaattctgtggcatattctaataattctattgctattccaacaaattttaccatttctgtgacaacagaaattttacctgtgtctatgacaaaaacatctgtggattgtaccatgtacatttgtggagattctacagaatgttctaatctgctgctgcagtatggatctttttgtacacagctgaatagagctttaacaggaattgctgtggaacaggataaaaatacacaggaagtgtttgctcaggtgaaacagatttacaaaacaccaccaattaaagattttggaggatttaattttagccagattctgcctgatccttctaaaccttctaaaagatcttttattgaagatctgctgtttaataaagtgacactggcagatgcaggatttattaaacagtatggagattgcctgggtgatattgctgcaagagatctgatttgtgctcagaaatttaatggactgacagtgctgcctcctctgctgacagatgaaatgattgctcagtacacatctgctttactggctggaacaattacaagcggatggacatttggagctggagctgctctgcagattccttttgcaatgcagatggcttacagatttaatggaattggagtgacacagaatgtgttatatgaaaatcagaaactgattgcaaatcagtttaattctgcaattggcaaaattcaggattctctgtcttctacagcttctgctctgggaaaactgcaggatgtggtgaatcagaatgcacaggcactgaatactctggtgaaacagctgtctagcaattttggggcaatttcttctgtgctgaatgatattctgtctagactggatcctcctgaagctgaagtgcagattgatagactgatcacaggaagactgcagtctctgcagacttatgtgacacagcagctgattagagctgctgaaattagagcttctgctaatctggctgctacaaaaatgtctgaatgtgtgctgggacagtcaaaaagagtggatttttgtggaaaaggatatcatctgatgtcttttccacagtctgctccacatggagtggtgtttttacatgtgacatatgtgccagcacaggaaaagaattttaccacagcaccagcaatttgtcatgatggaaaagcacattttccaagagaaggagtgtttgtgtctaatggaacacattggtttgtgacacagagaaatttttatgaacctcagattattacaacagataatacatttgtgtcaggaaattgtgatgtggtgattggaattgtgaataatacagtgtatgatccactgcagccagaactggattcttttaaagaagaactggataaatattttaaaaatcacacatctcctgatgtggatttaggagatatttctggaatcaatgcatctgtggtgaatattcagaaagaaattgatagactgaatgaagtggccaaaaatctgaatgaatctctgattgatctgcaggaacttggaaaatatgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacaca

SEQ ID NO: 9 - RNA, 3819 nt, synthetic construct
  source 1..3819: mol_type = other RNA
atgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacaca

SEQ ID NO: 10 - protein, 41 aa, synthetic construct
GSGYIPEAPRDGQAYVRKDGEWVLLSTFLGRSLEVLFQGPG

SEQ ID NO: 11 - RNA, 123 nt, synthetic construct
  source 1..123: mol_type = other RNA
ggatctggttatattcctgaagctccaagagatgggcaagcttacgttcgtaaagatggcgaatgggtattactttctacctttttaggccggtccctggaggtgctgttccagggccccggc

SEQ ID NO: 12 - RNA, 47 nt, synthetic construct
  source 1..47: mol_type = other RNA
aactagtattcttctggtccccacagactcagagagaacccgccacc

SEQ ID NO: 13 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 14 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 15 - RNA, 4282 nt, synthetic construct
  source 1..4282: mol_type = other RNA
gggcgaactagtattcttctggtccccacagactcagagagaacccgccaccatgtttgtgtttcttgtgctgctgcctcttgtgtcttctcagtgtgtgaatttgacaacaagaacacagctgccaccagcttatacaaattcttttaccagaggagtgtattatcctgataaagtgtttagatcttctgtgctgcacagcacacaggacctgtttctgccattttttagcaatgtgacatggtttcatgcaattcatgtgtctggaacaaatggaacaaaaagatttgataatcctgtgctgccttttaatgatggagtgtattttgcttcaacagaaaagtcaaatattattagaggatggatttttggaacaacactggattctaaaacacagtctctgctgattgtgaataatgcaacaaatgtggtgattaaagtgtgtgaatttcagttttgtaatgatccttttctgggagtgtattatcacaaaaataataaatcttggatggaatctgaatttagagtgtattcctctgcaaataattgtacatttgaatatgtgtctcagccttttctgatggatctggaaggaaaacagggcaattttaaaaatctgagagaatttgtgtttaaaaatattgatggatattttaaaatttattctaaacacacaccaattaatttagtgagagatctgcctcagggattttctgctctggaacctctggtggatctgccaattggcattaatattacaagatttcagacactgctggctctgcacagatcttatctgacacctggagattcttcttctggatggacagccggagctgcagcttattatgtgggctatctgcagccaagaacatttctgctgaaatataatgaaaatggaacaattacagatgctgtggattgtgctctggatcctctgtctgaaacaaaatgtacattaaaatcttttacagtggaaaaaggcatttatcagacatctaattttagagtgcagccaacagaatctattgtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaaaatctacaaatttagtgaaaaataaatgtgtgaattttaattttaatggattaacaggaacaggagtgctgacagaatctaataaaaaatttctgccttttcagcagtttggcagagatattgcagataccacagatgcagtgagagatcctcagacattagaaattctggatattacaccttgttcttttgggggtgtgtctgtgattacacctggaacaaatacatctaatcaggtggctgtgctgtatcaggatgtgaattgtacagaagtgccagtggcaattcatgcagatcagctgacaccaacatggagagtgtattctacaggatctaatgtgtttcagacaagagcaggatgtctgattggagcagaacatgtgaataattcttatgaatgtgatattccaattggagcaggcatttgtgcatcttatcagacacagacaaattccccaaggagagcaagatctgtggcatctcagtctattattgcatacaccatgtctctgggagcagaaaattctgtggcatattctaataattctattgctattccaacaaattttaccatttctgtgacaacagaaattttacctgtgtctatgacaaaaacatctgtggattgtaccatgtacatttgtggagattctacagaatgttctaatctgctgctgcagtatggatctttttgtacacagctgaatagagctttaacaggaattgctgtggaacaggataaaaatacacaggaagtgtttgctcaggtgaaacagatttacaaaacaccaccaattaaagattttggaggatttaattttagccagattctgcctgatccttctaaaccttctaaaagatcttttattgaagatctgctgtttaataaagtgacactggcagatgcaggatttattaaacagtatggagattgcctgggtgatattgctgcaagagatctgatttgtgctcagaaatttaatggactgacagtgctgcctcctctgctgacagatgaaatgattgctcagtacacatctgctttactggctggaacaattacaagcggatggacatttggagctggagctgctctgcagattccttttgcaatgcagatggcttacagatttaatggaattggagtgacacagaatgtgttatatgaaaatcagaaactgattgcaaatcagtttaattctgcaattggcaaaattcaggattctctgtcttctacagcttctgctctgggaaaactgcaggatgtggtgaatcagaatgcacaggcactgaatactctggtgaaacagctgtctagcaattttggggcaatttcttctgtgctgaatgatattctgtctagactggatcctcctgaagctgaagtgcagattgatagactgatcacaggaagactgcagtctctgcagacttatgtgacacagcagctgattagagctgctgaaattagagcttctgctaatctggctgctacaaaaatgtctgaatgtgtgctgggacagtcaaaaagagtggatttttgtggaaaaggatatcatctgatgtcttttccacagtctgctccacatggagtggtgtttttacatgtgacatatgtgccagcacaggaaaagaattttaccacagcaccagcaatttgtcatgatggaaaagcacattttccaagagaaggagtgtttgtgtctaatggaacacattggtttgtgacacagagaaatttttatgaacctcagattattacaacagataatacatttgtgtcaggaaattgtgatgtggtgattggaattgtgaataatacagtgtatgatccactgcagccagaactggattcttttaaagaagaactggataaatattttaaaaatcacacatctcctgatgtggatttaggagatatttctggaatcaatgcatctgtggtgaatattcagaaagaaattgatagactgaatgaagtggccaaaaatctgaatgaatctctgattgatctgcaggaacttggaaaatatgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 16 - RNA, 4282 nt, synthetic construct
  source 1..4282: mol_type = other RNA
gggcgaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 17 - RNA, 1261 nt, synthetic construct
  source 1..1261: mol_type = other RNA
gggcgaactagtattcttctggtccccacagactcagagagaacccgccaccatgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggccggtccctggaggtgctgttccagggccccggctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 18 - protein, 250 aa, synthetic construct
VRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 19 - RNA, 4283 nt, synthetic construct
  source 1..4283: mol_type = other RNA
agaataaactagtattcttctggtccccacagactcagagagaacccgccaccatgtttgtgtttcttgtgctgctgcctcttgtgtcttctcagtgtgtgaatttgacaacaagaacacagctgccaccagcttatacaaattcttttaccagaggagtgtattatcctgataaagtgtttagatcttctgtgctgcacagcacacaggacctgtttctgccattttttagcaatgtgacatggtttcatgcaattcatgtgtctggaacaaatggaacaaaaagatttgataatcctgtgctgccttttaatgatggagtgtattttgcttcaacagaaaagtcaaatattattagaggatggatttttggaacaacactggattctaaaacacagtctctgctgattgtgaataatgcaacaaatgtggtgattaaagtgtgtgaatttcagttttgtaatgatccttttctgggagtgtattatcacaaaaataataaatcttggatggaatctgaatttagagtgtattcctctgcaaataattgtacatttgaatatgtgtctcagccttttctgatggatctggaaggaaaacagggcaattttaaaaatctgagagaatttgtgtttaaaaatattgatggatattttaaaatttattctaaacacacaccaattaatttagtgagagatctgcctcagggattttctgctctggaacctctggtggatctgccaattggcattaatattacaagatttcagacactgctggctctgcacagatcttatctgacacctggagattcttcttctggatggacagccggagctgcagcttattatgtgggctatctgcagccaagaacatttctgctgaaatataatgaaaatggaacaattacagatgctgtggattgtgctctggatcctctgtctgaaacaaaatgtacattaaaatcttttacagtggaaaaaggcatttatcagacatctaattttagagtgcagccaacagaatctattgtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaaaatctacaaatttagtgaaaaataaatgtgtgaattttaattttaatggattaacaggaacaggagtgctgacagaatctaataaaaaatttctgccttttcagcagtttggcagagatattgcagataccacagatgcagtgagagatcctcagacattagaaattctggatattacaccttgttcttttgggggtgtgtctgtgattacacctggaacaaatacatctaatcaggtggctgtgctgtatcaggatgtgaattgtacagaagtgccagtggcaattcatgcagatcagctgacaccaacatggagagtgtattctacaggatctaatgtgtttcagacaagagcaggatgtctgattggagcagaacatgtgaataattcttatgaatgtgatattccaattggagcaggcatttgtgcatcttatcagacacagacaaattccccaaggagagcaagatctgtggcatctcagtctattattgcatacaccatgtctctgggagcagaaaattctgtggcatattctaataattctattgctattccaacaaattttaccatttctgtgacaacagaaattttacctgtgtctatgacaaaaacatctgtggattgtaccatgtacatttgtggagattctacagaatgttctaatctgctgctgcagtatggatctttttgtacacagctgaatagagctttaacaggaattgctgtggaacaggataaaaatacacaggaagtgtttgctcaggtgaaacagatttacaaaacaccaccaattaaagattttggaggatttaattttagccagattctgcctgatccttctaaaccttctaaaagatcttttattgaagatctgctgtttaataaagtgacactggcagatgcaggatttattaaacagtatggagattgcctgggtgatattgctgcaagagatctgatttgtgctcagaaatttaatggactgacagtgctgcctcctctgctgacagatgaaatgattgctcagtacacatctgctttactggctggaacaattacaagcggatggacatttggagctggagctgctctgcagattccttttgcaatgcagatggcttacagatttaatggaattggagtgacacagaatgtgttatatgaaaatcagaaactgattgcaaatcagtttaattctgcaattggcaaaattcaggattctctgtcttctacagcttctgctctgggaaaactgcaggatgtggtgaatcagaatgcacaggcactgaatactctggtgaaacagctgtctagcaattttggggcaatttcttctgtgctgaatgatattctgtctagactggatcctcctgaagctgaagtgcagattgatagactgatcacaggaagactgcagtctctgcagacttatgtgacacagcagctgattagagctgctgaaattagagcttctgctaatctggctgctacaaaaatgtctgaatgtgtgctgggacagtcaaaaagagtggatttttgtggaaaaggatatcatctgatgtcttttccacagtctgctccacatggagtggtgtttttacatgtgacatatgtgccagcacaggaaaagaattttaccacagcaccagcaatttgtcatgatggaaaagcacattttccaagagaaggagtgtttgtgtctaatggaacacattggtttgtgacacagagaaatttttatgaacctcagattattacaacagataatacatttgtgtcaggaaattgtgatgtggtgattggaattgtgaataatacagtgtatgatccactgcagccagaactggattcttttaaagaagaactggataaatattttaaaaatcacacatctcctgatgtggatttaggagatatttctggaatcaatgcatctgtggtgaatattcagaaagaaattgatagactgaatgaagtggccaaaaatctgaatgaatctctgattgatctgcaggaacttggaaaatatgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 20 - RNA, 4283 nt, synthetic construct
  source 1..4283: mol_type = other RNA
agaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 21 - RNA, 1262 nt, synthetic construct
  source 1..1262: mol_type = other RNA
agaataaactagtattcttctggtccccacagactcagagagaacccgccaccatgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggccggtccctggaggtgctgttccagggccccggctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 22 - protein, 1879 aa, synthetic construct
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQ

SEQ ID NO: 23 - protein, 613 aa, synthetic construct
RFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 24 - RNA, 11917 nt, synthetic construct
  source 1..11917: mol_type = other RNA
gatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgaaaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagagctagtcactgggctagggctcacaggcgagctggtcgatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaggcttttgcttgtcatgcaggtactctcagagcgctcatagccattataagacctaaaaaggcagtgctctgcggagatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggtagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagctaagcatgttgaccaagaaagcatgtctgcatctgaatcccggcggaacctgtgtcagcataggttatggttacgctgacagggccagcgaaagcatcattggtgctatagcgcggcagttcaagttttcccgagtatgcaaaccgaaatcctcacttgaggagacggaagttctgtttgtattcattgggtacgatcgcaaggcccgtacgcacaatccttacaagctatcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagtttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgattcttcagtgacagaacctgatgcagagctggtgagggtgcatcccaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaggagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaagaagaagaagatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctccagaaccagcctagtttccaccccgccaggcgtgaatagggtgatcactagagaggagctcgaagcgcttaccccgtcacgcactcctagcaggtcggtctccagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagctaggcaaacctctggcagcagacgatgaacatgatgatgacaggagaagggcattgcatgaggagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagactagtatgtttgtgtttcttgtgctgctgcctcttgtgtcttctcagtgtgtgaatttgacaacaagaacacagctgccaccagcttatacaaattcttttaccagaggagtgtattatcctgataaagtgtttagatcttctgtgctgcacagcacacaggacctgtttctgccattttttagcaatgtgacatggtttcatgcaattcatgtgtctggaacaaatggaacaaaaagatttgataatcctgtgctgccttttaatgatggagtgtattttgcttcaacagaaaagtcaaatattattagaggatggatttttggaacaacactggattctaaaacacagtctctgctgattgtgaataatgcaacaaatgtggtgattaaagtgtgtgaatttcagttttgtaatgatccttttctgggagtgtattatcacaaaaataataaatcttggatggaatctgaatttagagtgtattcctctgcaaataattgtacatttgaatatgtgtctcagccttttctgatggatctggaaggaaaacagggcaattttaaaaatctgagagaatttgtgtttaaaaatattgatggatattttaaaatttattctaaacacacaccaattaatttagtgagagatctgcctcagggattttctgctctggaacctctggtggatctgccaattggcattaatattacaagatttcagacactgctggctctgcacagatcttatctgacacctggagattcttcttctggatggacagccggagctgcagcttattatgtgggctatctgcagccaagaacatttctgctgaaatataatgaaaatggaacaattacagatgctgtggattgtgctctggatcctctgtctgaaacaaaatgtacattaaaatcttttacagtggaaaaaggcatttatcagacatctaattttagagtgcagccaacagaatctattgtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaaaatctacaaatttagtgaaaaataaatgtgtgaattttaattttaatggattaacaggaacaggagtgctgacagaatctaataaaaaatttctgccttttcagcagtttggcagagatattgcagataccacagatgcagtgagagatcctcagacattagaaattctggatattacaccttgttcttttgggggtgtgtctgtgattacacctggaacaaatacatctaatcaggtggctgtgctgtatcaggatgtgaattgtacagaagtgccagtggcaattcatgcagatcagctgacaccaacatggagagtgtattctacaggatctaatgtgtttcagacaagagcaggatgtctgattggagcagaacatgtgaataattcttatgaatgtgatattccaattggagcaggcatttgtgcatcttatcagacacagacaaattccccaaggagagcaagatctgtggcatctcagtctattattgcatacaccatgtctctgggagcagaaaattctgtggcatattctaataattctattgctattccaacaaattttaccatttctgtgacaacagaaattttacctgtgtctatgacaaaaacatctgtggattgtaccatgtacatttgtggagattctacagaatgttctaatctgctgctgcagtatggatctttttgtacacagctgaatagagctttaacaggaattgctgtggaacaggataaaaatacacaggaagtgtttgctcaggtgaaacagatttacaaaacaccaccaattaaagattttggaggatttaattttagccagattctgcctgatccttctaaaccttctaaaagatcttttattgaagatctgctgtttaataaagtgacactggcagatgcaggatttattaaacagtatggagattgcctgggtgatattgctgcaagagatctgatttgtgctcagaaatttaatggactgacagtgctgcctcctctgctgacagatgaaatgattgctcagtacacatctgctttactggctggaacaattacaagcggatggacatttggagctggagctgctctgcagattccttttgcaatgcagatggcttacagatttaatggaattggagtgacacagaatgtgttatatgaaaatcagaaactgattgcaaatcagtttaattctgcaattggcaaaattcaggattctctgtcttctacagcttctgctctgggaaaactgcaggatgtggtgaatcagaatgcacaggcactgaatactctggtgaaacagctgtctagcaattttggggcaatttcttctgtgctgaatgatattctgtctagactggatcctcctgaagctgaagtgcagattgatagactgatcacaggaagactgcagtctctgcagacttatgtgacacagcagctgattagagctgctgaaattagagcttctgctaatctggctgctacaaaaatgtctgaatgtgtgctgggacagtcaaaaagagtggatttttgtggaaaaggatatcatctgatgtcttttccacagtctgctccacatggagtggtgtttttacatgtgacatatgtgccagcacaggaaaagaattttaccacagcaccagcaatttgtcatgatggaaaagcacattttccaagagaaggagtgtttgtgtctaatggaacacattggtttgtgacacagagaaatttttatgaacctcagattattacaacagataatacatttgtgtcaggaaattgtgatgtggtgattggaattgtgaataatacagtgtatgatccactgcagccagaactggattcttttaaagaagaactggataaatattttaaaaatcacacatctcctgatgtggatttaggagatatttctggaatcaatgcatctgtggtgaatattcagaaagaaattgatagactgaatgaagtggccaaaaatctgaatgaatctctgattgatctgcaggaacttggaaaatatgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgcggccgcatgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 25 - RNA, 11917 nt, synthetic construct
  source 1..11917: mol_type = other RNA
gatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgaaaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagagctagtcactgggctagggctcacaggcgagctggtcgatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaggcttttgcttgtcatgcaggtactctcagagcgctcatagccattataagacctaaaaaggcagtgctctgcggagatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggtagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagctaagcatgttgaccaagaaagcatgtctgcatctgaatcccggcggaacctgtgtcagcataggttatggttacgctgacagggccagcgaaagcatcattggtgctatagcgcggcagttcaagttttcccgagtatgcaaaccgaaatcctcacttgaggagacggaagttctgtttgtattcattgggtacgatcgcaaggcccgtacgcacaatccttacaagctatcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagtttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgattcttcagtgacagaacctgatgcagagctggtgagggtgcatcccaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaggagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaagaagaagaagatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctccagaaccagcctagtttccaccccgccaggcgtgaatagggtgatcactagagaggagctcgaagcgcttaccccgtcacgcactcctagcaggtcggtctccagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagctaggcaaacctctggcagcagacgatgaacatgatgatgacaggagaagggcattgcatgaggagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagactagtatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgcggccgcatgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 26 - RNA, 8896 nt, synthetic construct
  source 1..8896: mol_type = other RNA
gatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgaaaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagagctagtcactgggctagggctcacaggcgagctggtcgatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaggcttttgcttgtcatgcaggtactctcagagcgctcatagccattataagacctaaaaaggcagtgctctgcggagatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggtagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagctaagcatgttgaccaagaaagcatgtctgcatctgaatcccggcggaacctgtgtcagcataggttatggttacgctgacagggccagcgaaagcatcattggtgctatagcgcggcagttcaagttttcccgagtatgcaaaccgaaatcctcacttgaggagacggaagttctgtttgtattcattgggtacgatcgcaaggcccgtacgcacaatccttacaagctatcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagtttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgattcttcagtgacagaacctgatgcagagctggtgagggtgcatcccaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaggagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaagaagaagaagatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctccagaaccagcctagtttccaccccgccaggcgtgaatagggtgatcactagagaggagctcgaagcgcttaccccgtcacgcactcctagcaggtcggtctccagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagctaggcaaacctctggcagcagacgatgaacatgatgatgacaggagaagggcattgcatgaggagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagactagtatgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggccggtccctggaggtgctgttccagggccccggctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgcggccgcatgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 27 - RNA, 9079 nt, synthetic construct
  source 1..9079: mol_type = other RNA
gatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgaaaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagagctagtcactgggctagggctcacaggcgagctggtcgatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaggcttttgcttgtcatgcaggtactctcagagcgctcatagccattataagacctaaaaaggcagtgctctgcggagatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggtagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagctaagcatgttgaccaagaaagcatgtctgcatctgaatcccggcggaacctgtgtcagcataggttatggttacgctgacagggccagcgaaagcatcattggtgctatagcgcggcagttcaagttttcccgagtatgcaaaccgaaatcctcacttgaggagacggaagttctgtttgtattcattgggtacgatcgcaaggcccgtacgcacaatccttacaagctatcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagtttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgattcttcagtgacagaacctgatgcagagctggtgagggtgcatcccaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaggagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaagaagaagaagatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctccagaaccagcctagtttccaccccgccaggcgtgaatagggtgatcactagagaggagctcgaagcgcttaccccgtcacgcactcctagcaggtcggtctccagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagctaggcaaacctctggcagcagacgatgaacatgatgatgacaggagaagggcattgcatgaggagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagactagtatgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggaagcggcagcggatctgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgtaaatttgatgaagatgattctgaacctgtgttaaaaggagtgaaattgcattacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgcggccgcatgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 28 - protein, 327 aa, synthetic construct
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCCKFDEDDSEPVLKGVKLHYT

SEQ ID NO: 29 - protein, 311 aa, synthetic construct
MFVFLVLLPLVSSQCVNLTVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCC

SEQ ID NO: 30 - RNA, 1397 nt, synthetic construct
  source 1..1397: mol_type = other RNA
agaataaactagtattcttctggtccccacagactcagagagaacccgccaccatgtttgtgtttcttgtgctgctgcctcttgtgtcttctcagtgtgtgaatttgacagtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggaagcggcagcggatctgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 31 - protein, 314 aa, synthetic construct
MDWIWRILFLVGAATGAHSQMQVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGSGSGSEQYIKWPWYIWLGFIAGLIAIVMVTIMLCCMTSCCSCLKGCCSCGSCC

SEQ ID NO: 32 - RNA, 1406 nt, synthetic construct
  source 1..1406: mol_type = other RNA
agaataaactagtattcttctggtccccacagactcagagagaacccgccaccatggattggatttggagaatcctgttcctcgtgggagccgctacaggagcccactcccagatgcaggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggaagcggcagcggatctgaacagtacattaaatggccttggtacatttggcttggatttattgcaggattaattgcaattgtgatggtgacaattatgttatgttgtatgacatcatgttgttcttgtttaaaaggatgttgttcttgtggaagctgttgttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 33 - protein, 9 aa, synthetic construct
GSPGSGSGS

SEQ ID NO: 34 - protein, 6 aa, synthetic construct
GSGSGS

SEQ ID NO: 35 - protein, 9 aa, synthetic construct
YLQPRTFLL

SEQ ID NO: 36 - protein, 9 aa, synthetic construct
RLQSLQTYV

SEQ ID NO: 37 - protein, 9 aa, synthetic construct
QYIKWPWYI

SEQ ID NO: 38 - protein, 9 aa, synthetic construct
NYNYLYRLF

SEQ ID NO: 39 - protein, 10 aa, synthetic construct
KWPWYIWLGF

SEQ ID NO: 40 - protein, 9 aa, synthetic construct
QPTESIVRF

SEQ ID NO: 41 - protein, 9 aa, synthetic construct
IPFAMQMAY

SEQ ID NO: 42 - protein, 9 aa, synthetic construct
LPFNDGVYF

SEQ ID NO: 43 - protein, 9 aa, synthetic construct
GVYFASTEK

SEQ ID NO: 44 - protein, 9 aa, synthetic construct
CVADYSVLY

SEQ ID NO: 45 - protein, 9 aa, synthetic construct
KCYGVSPTK

SEQ ID NO: 46 - protein, 9 aa, synthetic construct
FQPTNGVGY

SEQ ID NO: 47 - protein, 9 aa, synthetic construct
GTHWFVTQR

SEQ ID NO: 48 - protein, 9 aa, synthetic construct
VYDPLQPEL

SEQ ID NO: 49 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 50 - RNA, 3816 nt, synthetic construct
  source 1..3816: mol_type = other RNA
atgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcatcgtgagagagcccgaggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccccttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacagctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcttcagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 51 - RNA, 4274 nt, synthetic construct
  source 1..4274: mol_type = other RNA
agaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcatcgtgagagagcccgaggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccccttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacagctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcttcagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 52 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 53 - RNA, 3816 nt, synthetic construct
  source 1..3816: mol_type = other RNA
atgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcatcgtgagagagcccgaggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccccttcttcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacagctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcttcagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 54 - RNA, 4274 nt, synthetic construct
  source 1..4274: mol_type = other RNA
agaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcatcgtgagagagcccgaggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccccttcttcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacagctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcttcagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 55 - protein, 1270 aa, synthetic construct
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 56 - RNA, 3817 nt, synthetic construct
  source 1..3817: mol_type = other RNA
atgttcgtgttcctggtgctgctgcctctggtgtccagccagtgtgtgaacttcaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcggggcctgcctcagggcttctctgctctggaacccctggtggatctgcccatcggcatcaacatcacccggtttcagacactgcacatcagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgtcgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgac

SEQ ID NO: 57 - RNA, 4274 nt, synthetic construct
  source 1..4274: mol_type = other RNA
agaataaactagtattcttctggtccccacagactcagagagaacccgccaccatgttcgtgttcctggtgctgctgcctctggtgtccagccagtgtgtgaacttcaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcggggcctgcctcagggcttctctgctctggaacccctggtggatctgcccatcggcatcaacatcacccggtttcagacactgcacatcagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgtcgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 58 - protein, 1270 aa, synthetic construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 59 - RNA, 3816 nt, synthetic construct
  source 1..3816: mol_type = other RNA
atgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacatacggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgacgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccaccggagagccagaagcgtggccagccagagcatcattgcctacacaatgtctctgggcgccgagaacagcgtggcctactccaacaactctatcgctatccccatcaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctggccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccacccacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 60 - RNA, 4274 nt, synthetic construct
  source 1..4274: mol_type = other RNA
agaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacatacggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgacgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccaccggagagccagaagcgtggccagccagagcatcattgcctacacaatgtctctgggcgccgagaacagcgtggcctactccaacaactctatcgctatccccatcaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctggccagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccacccacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgagatctgctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 61 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 62 - DNA, 3819 bp, synthetic construct
  source 1..3819: mol_type = other DNA
atgttcgtgttcctggtgctgctgcctctggtgtccagccagtgtgtgaacctgagaaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacaggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagcaggcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 63 - DNA, 4277 bp, synthetic construct
  source 1..4277: mol_type = other DNA
agaataaactagtattcttctggtccccacagactcagagagaacccgccaccatgttcgtgttcctggtgctgctgcctctggtgtccagccagtgtgtgaacctgagaaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacaggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagcaggcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcagaacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgatttcacctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 64 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 65 - RNA, 3816 nt, synthetic construct
  source 1..3816: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggcaggcttcaactgctacttcccactgaggtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 66 - DNA, 3816 bp, synthetic construct
  source 1..3816: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggcaggcttcaactgctacttcccactgaggtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 67 - RNA, 4274 nt, synthetic construct
  source 1..4274: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggcaggcttcaactgctacttcccactgaggtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 68 - DNA, 4274 bp, synthetic construct
  source 1..4274: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggcaggcttcaactgctacttcccactgaggtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 69 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVRQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 70 - RNA, 3810 nt, synthetic construct
  source 1..3810: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgcggcagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 71 - DNA, 3810 bp, synthetic construct
  source 1..3810: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgcggcagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 72 - RNA, 4268 nt, synthetic construct
  source 1..4268: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgcggcagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 73 - DNA, 4268 bp, synthetic construct
  source 1..4268: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgcggcagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 74 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 75 - RNA, 3810 nt, synthetic construct
  source 1..3810: mol_type = other RNA
atgttcgtgttcctggtgctgctgcctctggtgtccagccagtgtgtgaacctgatcaccagaacacagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggccggcgtgaactgctacttcccactgcagtcctacggctttaggcccacatatggcgtgggccatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 76 - DNA, 3810 bp, synthetic construct
  source 1..3810: mol_type = other DNA
atgttcgtgttcctggtgctgctgcctctggtgtccagccagtgtgtgaacctgatcaccagaacacagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtgtactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccccttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggccggcgtgaactgctacttcccactgcagtcctacggctttaggcccacatatggcgtgggccatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 77 - protein, 1268 aa, synthetic construct; identical to SEQ ID NO: 69

SEQ ID NO: 78 - RNA, 3810 nt, synthetic construct; identical to SEQ ID NO: 70
  source 1..3810: mol_type = other RNA

SEQ ID NO: 79 - DNA, 3810 bp, synthetic construct; identical to SEQ ID NO: 71
  source 1..3810: mol_type = other DNA

SEQ ID NO: 80 - protein, 566 aa, synthetic construct
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 81 - DNA, 1704 bp, synthetic construct
  source 1..1704: mol_type = other DNA
atgaagaccatcattgccctgagcaacatcctgtgcctggtgttcgcccagaagatccccggcaacgacaatagcaccgccacactgtgtctgggccatcacgctgtgcctaacggcaccatcgtgaaaaccatcaccaacgaccggatcgaagtgaccaacgccacagagctggtgcagaacagcagcatcggcgagatctgtggcagccctcaccagattctggacggcggcaattgcaccctgatcgatgctctgctgggcgaccctcagtgtgacggcttccagaacaaagaatgggacctgttcgtggaaagaagccgggccaacagcaactgctacccctacgatgtgcccgactacgccagcctgagatctctggtggcctctagcggcaccctggaattcaagaacgagagcttcaactggaccggcgtgaagcagaatggcaccagcagcgcctgtatcagaggcagcagctccagcttcttcagcagactgaactggctgaccagcctgaacaacatctaccccgctcagaacgtgaccatgcctaacaaagagcagttcgacaagctgtacatctggggcgtgcaccatcctgacaccgacaagaaccagatcagcctgtttgcccagagcagcggcagaatcaccgtgtccactaagagaagccagcaggccgtgattcccaacatcggcagcagaccccggatcagagacatccccagccggatcagcatctactggacaatcgtgaagcccggcgacatcctgctgatcaacagcaccggcaatctgatcgcccctcggggctacttcaagatcagaagcggcaagagcagcatcatgcggagcgacgcccctatcggcaagtgcaagagcgagtgcatcaccccaaacggcagcatccccaacgacaagcccttccagaatgtgaaccggatcacctacggcgcctgtcctagatacgtgaaacagagcaccctgaagctggccaccggcatgagaaacgtgccagagaagcagaccagaggcatcttcggagccattgccggcttcatcgagaacggctgggaaggcatggtggacggatggtacggcttcagacaccagaacagcgaaggcagaggacaggccgctgacctgaaatctacacaggccgccatcgaccagatcaacggcaagctgaaccggctgatcggcaagaccaacgagaagttccaccagatcgagaaagagttcagcgaggtcgagggcagagtgcaggacctcgagaaatacgtggaagataccaagatcgacctgtggtcctacaatgccgaactgctggtggccctggaaaaccagcacaccatcgacctgaccgacagcgagatgaacaagctgttcgaaaagaccaagaagcagctgcgcgagaacgccgaggatatgggcaacggctgctttaagatctaccacaagtgcgacaacgcctgcatcggctccatccggaacgagacatacgaccacaacgtgtacagagatgaggccctgaacaatcggttccagatcaaaggcgtggaactgaagtccggctacaaggactggatcctgtggatcagcttcgccatgagctgctttctgctgtgtatcgccctgctgggcttcatcatgtgggcctgccagaaaggcaacatcagatgcaacatctgcatctgatga

SEQ ID NO: 82 - RNA, 1704 nt, synthetic construct
  source 1..1704: mol_type = other RNA
atgaagaccatcattgccctgagcaacatcctgtgcctggtgttcgcccagaagatccccggcaacgacaatagcaccgccacactgtgtctgggccatcacgctgtgcctaacggcaccatcgtgaaaaccatcaccaacgaccggatcgaagtgaccaacgccacagagctggtgcagaacagcagcatcggcgagatctgtggcagccctcaccagattctggacggcggcaattgcaccctgatcgatgctctgctgggcgaccctcagtgtgacggcttccagaacaaagaatgggacctgttcgtggaaagaagccgggccaacagcaactgctacccctacgatgtgcccgactacgccagcctgagatctctggtggcctctagcggcaccctggaattcaagaacgagagcttcaactggaccggcgtgaagcagaatggcaccagcagcgcctgtatcagaggcagcagctccagcttcttcagcagactgaactggctgaccagcctgaacaacatctaccccgctcagaacgtgaccatgcctaacaaagagcagttcgacaagctgtacatctggggcgtgcaccatcctgacaccgacaagaaccagatcagcctgtttgcccagagcagcggcagaatcaccgtgtccactaagagaagccagcaggccgtgattcccaacatcggcagcagaccccggatcagagacatccccagccggatcagcatctactggacaatcgtgaagcccggcgacatcctgctgatcaacagcaccggcaatctgatcgcccctcggggctacttcaagatcagaagcggcaagagcagcatcatgcggagcgacgcccctatcggcaagtgcaagagcgagtgcatcaccccaaacggcagcatccccaacgacaagcccttccagaatgtgaaccggatcacctacggcgcctgtcctagatacgtgaaacagagcaccctgaagctggccaccggcatgagaaacgtgccagagaagcagaccagaggcatcttcggagccattgccggcttcatcgagaacggctgggaaggcatggtggacggatggtacggcttcagacaccagaacagcgaaggcagaggacaggccgctgacctgaaatctacacaggccgccatcgaccagatcaacggcaagctgaaccggctgatcggcaagaccaacgagaagttccaccagatcgagaaagagttcagcgaggtcgagggcagagtgcaggacctcgagaaatacgtggaagataccaagatcgacctgtggtcctacaatgccgaactgctggtggccctggaaaaccagcacaccatcgacctgaccgacagcgagatgaacaagctgttcgaaaagaccaagaagcagctgcgcgagaacgccgaggatatgggcaacggctgctttaagatctaccacaagtgcgacaacgcctgcatcggctccatccggaacgagacatacgaccacaacgtgtacagagatgaggccctgaacaatcggttccagatcaaaggcgtggaactgaagtccggctacaaggactggatcctgtggatcagcttcgccatgagctgctttctgctgtgtatcgccctgctgggcttcatcatgtgggcctgccagaaaggcaacatcagatgcaacatctgcatctgatga

SEQ ID NO: 83 - DNA, 2161 bp, synthetic construct
  source 1..2161: mol_type = other DNA
agaataaactagtattcttctggtccccacagactcagagagaacccgccaccatgaagaccatcattgccctgagcaacatcctgtgcctggtgttcgcccagaagatccccggcaacgacaatagcaccgccacactgtgtctgggccatcacgctgtgcctaacggcaccatcgtgaaaaccatcaccaacgaccggatcgaagtgaccaacgccacagagctggtgcagaacagcagcatcggcgagatctgtggcagccctcaccagattctggacggcggcaattgcaccctgatcgatgctctgctgggcgaccctcagtgtgacggcttccagaacaaagaatgggacctgttcgtggaaagaagccgggccaacagcaactgctacccctacgatgtgcccgactacgccagcctgagatctctggtggcctctagcggcaccctggaattcaagaacgagagcttcaactggaccggcgtgaagcagaatggcaccagcagcgcctgtatcagaggcagcagctccagcttcttcagcagactgaactggctgaccagcctgaacaacatctaccccgctcagaacgtgaccatgcctaacaaagagcagttcgacaagctgtacatctggggcgtgcaccatcctgacaccgacaagaaccagatcagcctgtttgcccagagcagcggcagaatcaccgtgtccactaagagaagccagcaggccgtgattcccaacatcggcagcagaccccggatcagagacatccccagccggatcagcatctactggacaatcgtgaagcccggcgacatcctgctgatcaacagcaccggcaatctgatcgcccctcggggctacttcaagatcagaagcggcaagagcagcatcatgcggagcgacgcccctatcggcaagtgcaagagcgagtgcatcaccccaaacggcagcatccccaacgacaagcccttccagaatgtgaaccggatcacctacggcgcctgtcctagatacgtgaaacagagcaccctgaagctggccaccggcatgagaaacgtgccagagaagcagaccagaggcatcttcggagccattgccggcttcatcgagaacggctgggaaggcatggtggacggatggtacggcttcagacaccagaacagcgaaggcagaggacaggccgctgacctgaaatctacacaggccgccatcgaccagatcaacggcaagctgaaccggctgatcggcaagaccaacgagaagttccaccagatcgagaaagagttcagcgaggtcgagggcagagtgcaggacctcgagaaatacgtggaagataccaagatcgacctgtggtcctacaatgccgaactgctggtggccctggaaaaccagcacaccatcgacctgaccgacagcgagatgaacaagctgttcgaaaagaccaagaagcagctgcgcgagaacgccgaggatatgggcaacggctgctttaagatctaccacaagtgcgacaacgcctgcatcggctccatccggaacgagacatacgaccacaacgtgtacagagatgaggccctgaacaatcggttccagatcaaaggcgtggaactgaagtccggctacaaggactggatcctgtggatcagcttcgccatgagctgctttctgctgtgtatcgccctgctgggcttcatcatgtgggcctgccagaaaggcaacatcagatgcaacatctgcatctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 84 - RNA, 2161 nt, synthetic construct
  source 1..2161: mol_type = other RNA
agaataaactagtattcttctggtccccacagactcagagagaacccgccaccatgaagaccatcattgccctgagcaacatcctgtgcctggtgttcgcccagaagatccccggcaacgacaatagcaccgccacactgtgtctgggccatcacgctgtgcctaacggcaccatcgtgaaaaccatcaccaacgaccggatcgaagtgaccaacgccacagagctggtgcagaacagcagcatcggcgagatctgtggcagccctcaccagattctggacggcggcaattgcaccctgatcgatgctctgctgggcgaccctcagtgtgacggcttccagaacaaagaatgggacctgttcgtggaaagaagccgggccaacagcaactgctacccctacgatgtgcccgactacgccagcctgagatctctggtggcctctagcggcaccctggaattcaagaacgagagcttcaactggaccggcgtgaagcagaatggcaccagcagcgcctgtatcagaggcagcagctccagcttcttcagcagactgaactggctgaccagcctgaacaacatctaccccgctcagaacgtgaccatgcctaacaaagagcagttcgacaagctgtacatctggggcgtgcaccatcctgacaccgacaagaaccagatcagcctgtttgcccagagcagcggcagaatcaccgtgtccactaagagaagccagcaggccgtgattcccaacatcggcagcagaccccggatcagagacatccccagccggatcagcatctactggacaatcgtgaagcccggcgacatcctgctgatcaacagcaccggcaatctgatcgcccctcggggctacttcaagatcagaagcggcaagagcagcatcatgcggagcgacgcccctatcggcaagtgcaagagcgagtgcatcaccccaaacggcagcatccccaacgacaagcccttccagaatgtgaaccggatcacctacggcgcctgtcctagatacgtgaaacagagcaccctgaagctggccaccggcatgagaaacgtgccagagaagcagaccagaggcatcttcggagccattgccggcttcatcgagaacggctgggaaggcatggtggacggatggtacggcttcagacaccagaacagcgaaggcagaggacaggccgctgacctgaaatctacacaggccgccatcgaccagatcaacggcaagctgaaccggctgatcggcaagaccaacgagaagttccaccagatcgagaaagagttcagcgaggtcgagggcagagtgcaggacctcgagaaatacgtggaagataccaagatcgacctgtggtcctacaatgccgaactgctggtggccctggaaaaccagcacaccatcgacctgaccgacagcgagatgaacaagctgttcgaaaagaccaagaagcagctgcgcgagaacgccgaggatatgggcaacggctgctttaagatctaccacaagtgcgacaacgcctgcatcggctccatccggaacgagacatacgaccacaacgtgtacagagatgaggccctgaacaatcggttccagatcaaaggcgtggaactgaagtccggctacaaggactggatcctgtggatcagcttcgccatgagctgctttctgctgtgtatcgccctgctgggcttcatcatgtgggcctgccagaaaggcaacatcagatgcaacatctgcatctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 85 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 86 - DNA, 1752 bp, synthetic construct
  source 1..1752: mol_type = other DNA
atgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagccacttcgccaacctgaagggcaccgagacaagaggcaagctgtgccccaagtgcctgaactgcaccgatctggatgtggccctgggcagacctaagtgtaccggcaagatccctagcgccagagtgtccatcctgcacgaagtgcggcctgtgaccagcggctgctttcccattatgcacgaccggaccaagatcagacagctgcccaatctgctgcggggctatgaacatgtgcggctgagcacccacaacgtgatcaacacagaggatgcccctggcggcccttacgagatcggaacatctggctcttgcctgaacattaccaacggcaagggcttcttcgccaccatggcttgggccgtgcctaagaacaagaccgccaccaatccactgaccatcgaggtgccctacatctgtaccgaggaagaagatcagatcaccgtctggggcttccacagcgacgacgaaacacagatggccagactgtacggcgacagcaagcctcagaagttcaccagctctgccaacggcgtgaccacacactacgtgtcccagatcggcggcttccccaatcagacagaagatggcggactgccccagagcggaagaatcgtggtggactacatggtgcagaagtccggcaagaccggcacaatcacataccagcggggaatcctgctgcctcagaaagtttggtgcgccagcggcaagagcaaagtgatcaagggcagcctgcctctgatcggcgaagccgattgtctgcacgagaagtacggcggcctgaacaagagcaagccctactacacaggcgagcacgccaaggccatcggcaactgtcctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggcccaagcgccgtggaaatcggcaatggctgcttcgagacaaagcacaagtgcaaccagacctgcctggacagaattgccgccggaacatttgacgccggcgagtttagcctgcctaccttcgactccctgaacatcacagccgccagcctgaatgacgacggcctggacaatcacaccatcctgctgtactactccaccgccgcttctagcctggccgtgacactgatgatcgccatctttgtggtgtacatggtgtccagagacaacgtgtcctgcagcatctgcctgtgatga

SEQ ID NO: 87 - RNA, 1752 nt, synthetic construct
  source 1..1752: mol_type = other RNA
atgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagccacttcgccaacctgaagggcaccgagacaagaggcaagctgtgccccaagtgcctgaactgcaccgatctggatgtggccctgggcagacctaagtgtaccggcaagatccctagcgccagagtgtccatcctgcacgaagtgcggcctgtgaccagcggctgctttcccattatgcacgaccggaccaagatcagacagctgcccaatctgctgcggggctatgaacatgtgcggctgagcacccacaacgtgatcaacacagaggatgcccctggcggcccttacgagatcggaacatctggctcttgcctgaacattaccaacggcaagggcttcttcgccaccatggcttgggccgtgcctaagaacaagaccgccaccaatccactgaccatcgaggtgccctacatctgtaccgaggaagaagatcagatcaccgtctggggcttccacagcgacgacgaaacacagatggccagactgtacggcgacagcaagcctcagaagttcaccagctctgccaacggcgtgaccacacactacgtgtcccagatcggcggcttccccaatcagacagaagatggcggactgccccagagcggaagaatcgtggtggactacatggtgcagaagtccggcaagaccggcacaatcacataccagcggggaatcctgctgcctcagaaagtttggtgcgccagcggcaagagcaaagtgatcaagggcagcctgcctctgatcggcgaagccgattgtctgcacgagaagtacggcggcctgaacaagagcaagccctactacacaggcgagcacgccaaggccatcggcaactgtcctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggcccaagcgccgtggaaatcggcaatggctgcttcgagacaaagcacaagtgcaaccagacctgcctggacagaattgccgccggaacatttgacgccggcgagtttagcctgcctaccttcgactccctgaacatcacagccgccagcctgaatgacgacggcctggacaatcacaccatcctgctgtactactccaccgccgcttctagcctggccgtgacactgatgatcgccatctttgtggtgtacatggtgtccagagacaacgtgtcctgcagcatctgcctgtgatga

SEQ ID NO: 88 - DNA, 2209 bp, synthetic construct
  source 1..2209: mol_type = other DNA
agaataaactagtattcttctggtccccacagactcagagagaacccgccaccatgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagccacttcgccaacctgaagggcaccgagacaagaggcaagctgtgccccaagtgcctgaactgcaccgatctggatgtggccctgggcagacctaagtgtaccggcaagatccctagcgccagagtgtccatcctgcacgaagtgcggcctgtgaccagcggctgctttcccattatgcacgaccggaccaagatcagacagctgcccaatctgctgcggggctatgaacatgtgcggctgagcacccacaacgtgatcaacacagaggatgcccctggcggcccttacgagatcggaacatctggctcttgcctgaacattaccaacggcaagggcttcttcgccaccatggcttgggccgtgcctaagaacaagaccgccaccaatccactgaccatcgaggtgccctacatctgtaccgaggaagaagatcagatcaccgtctggggcttccacagcgacgacgaaacacagatggccagactgtacggcgacagcaagcctcagaagttcaccagctctgccaacggcgtgaccacacactacgtgtcccagatcggcggcttccccaatcagacagaagatggcggactgccccagagcggaagaatcgtggtggactacatggtgcagaagtccggcaagaccggcacaatcacataccagcggggaatcctgctgcctcagaaagtttggtgcgccagcggcaagagcaaagtgatcaagggcagcctgcctctgatcggcgaagccgattgtctgcacgagaagtacggcggcctgaacaagagcaagccctactacacaggcgagcacgccaaggccatcggcaactgtcctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggcccaagcgccgtggaaatcggcaatggctgcttcgagacaaagcacaagtgcaaccagacctgcctggacagaattgccgccggaacatttgacgccggcgagtttagcctgcctaccttcgactccctgaacatcacagccgccagcctgaatgacgacggcctggacaatcacaccatcctgctgtactactccaccgccgcttctagcctggccgtgacactgatgatcgccatctttgtggtgtacatggtgtccagagacaacgtgtcctgcagcatctgcctgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 89 - RNA, 2209 nt, synthetic construct
  source 1..2209: mol_type = other RNA
agaataaactagtattcttctggtccccacagactcagagagaacccgccaccatgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagccacttcgccaacctgaagggcaccgagacaagaggcaagctgtgccccaagtgcctgaactgcaccgatctggatgtggccctgggcagacctaagtgtaccggcaagatccctagcgccagagtgtccatcctgcacgaagtgcggcctgtgaccagcggctgctttcccattatgcacgaccggaccaagatcagacagctgcccaatctgctgcggggctatgaacatgtgcggctgagcacccacaacgtgatcaacacagaggatgcccctggcggcccttacgagatcggaacatctggctcttgcctgaacattaccaacggcaagggcttcttcgccaccatggcttgggccgtgcctaagaacaagaccgccaccaatccactgaccatcgaggtgccctacatctgtaccgaggaagaagatcagatcaccgtctggggcttccacagcgacgacgaaacacagatggccagactgtacggcgacagcaagcctcagaagttcaccagctctgccaacggcgtgaccacacactacgtgtcccagatcggcggcttccccaatcagacagaagatggcggactgccccagagcggaagaatcgtggtggactacatggtgcagaagtccggcaagaccggcacaatcacataccagcggggaatcctgctgcctcagaaagtttggtgcgccagcggcaagagcaaagtgatcaagggcagcctgcctctgatcggcgaagccgattgtctgcacgagaagtacggcggcctgaacaagagcaagccctactacacaggcgagcacgccaaggccatcggcaactgtcctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggcccaagcgccgtggaaatcggcaatggctgcttcgagacaaagcacaagtgcaaccagacctgcctggacagaattgccgccggaacatttgacgccggcgagtttagcctgcctaccttcgactccctgaacatcacagccgccagcctgaatgacgacggcctggacaatcacaccatcctgctgtactactccaccgccgcttctagcctggccgtgacactgatgatcgccatctttgtggtgtacatggtgtccagagacaacgtgtcctgcagcatctgcctgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 90 - protein, 566 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 91 - DNA, 1704 bp, synthetic construct
  source 1..1704: mol_type = other DNA
atgaaggccatcctggtggtcatgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggatcctgggcaaccctgagtgtgaaagcctgagcaccgccagatcctggtcctacatcgtggaaaccagcaacagcgacaacggcacatgctaccccggcgacttcatcaactacgaggaactgcgggaacagctgagcagcgtgtccagcttcgagagattcgagatcttccccaagaccagcagctggcccaaccacgactctgacaatggcgtgacagccgcctgtcctcatgccggcgctaagagcttctacaagaacctgatctggctggtcaagaagggcaagagctaccccaagatcaaccagacctacatcaacgacaagggcaaagaggtgctggtcctctggggcatccaccatcctccaacaatcgccgatcagcagagcctgtaccagaacgccgatgcctatgtgttcgtgggcaccagccggtacagcaagaagttcaagcccgagatcgccaccaggcctaaagtgcgggatcaagagggcagaatgaactactactggaccctggtggaacccggcgacaagatcacatttgaggccaccggcaatctggtggcccctagatacgccttcaccatggaaagagatgccggcagcggcatcatcatcagcgatacccctgtgcacgactgcaacaccacctgtcagacacctgagggcgccatcaataccagcctgcctttccagaacgtgcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctgctggagaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagatcgacggcgtgaagctggatagcacccggatctaccagattctggccatctacagcaccgtggcctctagcctggtgctggtggtttctctgggcgctatcagcttctggatgtgcagcaatggcagcctgcagtgccggatctgcatctgatga

SEQ ID NO: 92 - RNA, 1704 nt, synthetic construct
  source 1..1704: mol_type = other RNA
atgaaggccatcctggtggtcatgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggatcctgggcaaccctgagtgtgaaagcctgagcaccgccagatcctggtcctacatcgtggaaaccagcaacagcgacaacggcacatgctaccccggcgacttcatcaactacgaggaactgcgggaacagctgagcagcgtgtccagcttcgagagattcgagatcttccccaagaccagcagctggcccaaccacgactctgacaatggcgtgacagccgcctgtcctcatgccggcgctaagagcttctacaagaacctgatctggctggtcaagaagggcaagagctaccccaagatcaaccagacctacatcaacgacaagggcaaagaggtgctggtcctctggggcatccaccatcctccaacaatcgccgatcagcagagcctgtaccagaacgccgatgcctatgtgttcgtgggcaccagccggtacagcaagaagttcaagcccgagatcgccaccaggcctaaagtgcgggatcaagagggcagaatgaactactactggaccctggtggaacccggcgacaagatcacatttgaggccaccggcaatctggtggcccctagatacgccttcaccatggaaagagatgccggcagcggcatcatcatcagcgatacccctgtgcacgactgcaacaccacctgtcagacacctgagggcgccatcaataccagcctgcctttccagaacgtgcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctgctggagaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagatcgacggcgtgaagctggatagcacccggatctaccagattctggccatctacagcaccgtggcctctagcctggtgctggtggtttctctgggcgctatcagcttctggatgtgcagcaatggcagcctgcagtgccggatctgcatctgatga

SEQ ID NO: 93 - DNA, 2162 bp, synthetic construct
  source 1..2162: mol_type = other DNA
agaataaactagtattcttctggtccccacagactcagagagaacccgccaccatgaaggccatcctggtggtcatgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggatcctgggcaaccctgagtgtgaaagcctgagcaccgccagatcctggtcctacatcgtggaaaccagcaacagcgacaacggcacatgctaccccggcgacttcatcaactacgaggaactgcgggaacagctgagcagcgtgtccagcttcgagagattcgagatcttccccaagaccagcagctggcccaaccacgactctgacaatggcgtgacagccgcctgtcctcatgccggcgctaagagcttctacaagaacctgatctggctggtcaagaagggcaagagctaccccaagatcaaccagacctacatcaacgacaagggcaaagaggtgctggtcctctggggcatccaccatcctccaacaatcgccgatcagcagagcctgtaccagaacgccgatgcctatgtgttcgtgggcaccagccggtacagcaagaagttcaagcccgagatcgccaccaggcctaaagtgcgggatcaagagggcagaatgaactactactggaccctggtggaacccggcgacaagatcacatttgaggccaccggcaatctggtggcccctagatacgccttcaccatggaaagagatgccggcagcggcatcatcatcagcgatacccctgtgcacgactgcaacaccacctgtcagacacctgagggcgccatcaataccagcctgcctttccagaacgtgcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctgctggagaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagatcgacggcgtgaagctggatagcacccggatctaccagattctggccatctacagcaccgtggcctctagcctggtgctggtggtttctctgggcgctatcagcttctggatgtgcagcaatggcagcctgcagtgccggatctgcatctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 94 - RNA, 2162 nt, synthetic construct
  source 1..2162: mol_type = other RNA
agaataaactagtattcttctggtccccacagactcagagagaacccgccaccatgaaggccatcctggtggtcatgctgtacaccttcaccaccgccaacgccgacacactgtgtatcggctaccacgccaacaacagcaccgacaccgtggataccgtgctggaaaagaacgtgaccgtgacacacagcgtgaacctgctggaagataagcacaacggcaagctgtgcaagctgagaggcgtggcacctctgcacctgggcaagtgtaatatcgccggctggatcctgggcaaccctgagtgtgaaagcctgagcaccgccagatcctggtcctacatcgtggaaaccagcaacagcgacaacggcacatgctaccccggcgacttcatcaactacgaggaactgcgggaacagctgagcagcgtgtccagcttcgagagattcgagatcttccccaagaccagcagctggcccaaccacgactctgacaatggcgtgacagccgcctgtcctcatgccggcgctaagagcttctacaagaacctgatctggctggtcaagaagggcaagagctaccccaagatcaaccagacctacatcaacgacaagggcaaagaggtgctggtcctctggggcatccaccatcctccaacaatcgccgatcagcagagcctgtaccagaacgccgatgcctatgtgttcgtgggcaccagccggtacagcaagaagttcaagcccgagatcgccaccaggcctaaagtgcgggatcaagagggcagaatgaactactactggaccctggtggaacccggcgacaagatcacatttgaggccaccggcaatctggtggcccctagatacgccttcaccatggaaagagatgccggcagcggcatcatcatcagcgatacccctgtgcacgactgcaacaccacctgtcagacacctgagggcgccatcaataccagcctgcctttccagaacgtgcaccccatcaccatcggcaagtgccccaaatacgtgaagtccaccaagctgaggctggccacaggcctgagaaatgtgccctccatccagagcagaggcctgtttggagccattgccggctttatcgaaggcggctggacaggcatggtggacggatggtacggataccaccaccagaacgagcaaggctctggctatgccgccgacctgaagtctacccagaatgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctacaatgccgaactgctggtgctgctggagaacgagagaaccctggactaccacgacagcaacgtgaagaacctgtacgagaaagtgcgcaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagatcgacggcgtgaagctggatagcacccggatctaccagattctggccatctacagcaccgtggcctctagcctggtgctggtggtttctctgggcgctatcagcttctggatgtgcagcaatggcagcctgcagtgccggatctgcatctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 95 - protein, 566 aa, synthetic construct [sequence omitted]

SEQ ID NO: 96 - DNA, 1704 bp, synthetic construct
  source 1..1704: mol_type = other DNA
atgaagaccatcattgccctgagctacatcctgtgcctggtgttcgcccagaagatccccggcaacgacaatagcaccgccacactgtgtctgggccatcacgctgtgcctaacggcaccatcgtgaaaaccatcaccaacgaccggatcgaagtgaccaacgccacagagctggtgcagaacagcagcatcggcgagatctgcgatagccctcaccagatcctggacggcggcaattgcacactgatcgatgccctgctgggcgaccctcagtgtgatggcttccagaacaaagaatgggacctgttcgtggaaagaagccgggccaacagcaactgctacccctacgatgtgcccgactacgccagcctgagatctctggtggcctctagcggcaccctggaattcaagaacgagagcttcaactggaccggcgtgaagcagaatggcaccagcagcgcctgtatcagaggcagcagctccagcttcttcagcagactgaactggctgacccacctgaactacacataccccgctctgaacgtgaccatgcctaacaacgagcagttcgacaagctgtacatctggggcgtgcaccatcctagcaccgacaaggatcagatcagcctgtttgcccagcctagcggcagaatcaccgtgtccactaagagaagccagcaggccgtgattcccaacatcggcagcagaccccggatcagagacatccccagccggatcagcatctactggacaatcgtgaagcccggcgacatcctgctgatcaacagcaccggcaatctgatcgcccctcggggctacttcaagatcagaagcggcaagagcagcatcatgcggagcgacgcccctatcggcaagtgcaagagcgagtgcatcaccccaaacggcagcatccccaacgacaagcccttccagaatgtgaaccggatcacctacggcgcctgtcctagatacgtgaaacagagcaccctgaagctggccaccggcatgagaaacgtgccagagaagcagaccagaggcatcttcggagccattgccggcttcatcgagaacggctgggaaggcatggtggacggatggtacggcttcagacaccagaacagcgaaggcagaggacaggccgctgacctgaaatctacacaggccgccatcgaccagatcaacggcaagctgaaccggctgatcggcaagaccaacgagaagttccaccagatcgagaaagagttcagcgaggtcgagggcagagtgcaggacctcgaaaaatacgtggaagataccaagatcgacctgtggtcctacaatgccgaactgctggtggccctggaaaaccagcacaccatcgacctgaccgacagcgagatgaacaagctgttcgaaaagaccaagaagcagctgcgcgagaacgccgaggatatgggcaacggctgctttaagatctaccacaagtgcgacaacgcctgcatcggctccatccggaacgagacatacgaccacaacgtgtacagagatgaggccctgaacaaccggttccagatcaaaggcgtggaactgaagtccggctacaaggactggatactgtggatcagcttcgccatgagctgctttctgctgtgtatcgctctgctgggcttcatcatgtgggcctgccagaaaggcaacatccggtgcaacatctgcatctgatga

SEQ ID NO: 97 - RNA, 1704 nt, synthetic construct
  source 1..1704: mol_type = other RNA
atgaagaccatcattgccctgagctacatcctgtgcctggtgttcgcccagaagatccccggcaacgacaatagcaccgccacactgtgtctgggccatcacgctgtgcctaacggcaccatcgtgaaaaccatcaccaacgaccggatcgaagtgaccaacgccacagagctggtgcagaacagcagcatcggcgagatctgcgatagccctcaccagatcctggacggcggcaattgcacactgatcgatgccctgctgggcgaccctcagtgtgatggcttccagaacaaagaatgggacctgttcgtggaaagaagccgggccaacagcaactgctacccctacgatgtgcccgactacgccagcctgagatctctggtggcctctagcggcaccctggaattcaagaacgagagcttcaactggaccggcgtgaagcagaatggcaccagcagcgcctgtatcagaggcagcagctccagcttcttcagcagactgaactggctgacccacctgaactacacataccccgctctgaacgtgaccatgcctaacaacgagcagttcgacaagctgtacatctggggcgtgcaccatcctagcaccgacaaggatcagatcagcctgtttgcccagcctagcggcagaatcaccgtgtccactaagagaagccagcaggccgtgattcccaacatcggcagcagaccccggatcagagacatccccagccggatcagcatctactggacaatcgtgaagcccggcgacatcctgctgatcaacagcaccggcaatctgatcgcccctcggggctacttcaagatcagaagcggcaagagcagcatcatgcggagcgacgcccctatcggcaagtgcaagagcgagtgcatcaccccaaacggcagcatccccaacgacaagcccttccagaatgtgaaccggatcacctacggcgcctgtcctagatacgtgaaacagagcaccctgaagctggccaccggcatgagaaacgtgccagagaagcagaccagaggcatcttcggagccattgccggcttcatcgagaacggctgggaaggcatggtggacggatggtacggcttcagacaccagaacagcgaaggcagaggacaggccgctgacctgaaatctacacaggccgccatcgaccagatcaacggcaagctgaaccggctgatcggcaagaccaacgagaagttccaccagatcgagaaagagttcagcgaggtcgagggcagagtgcaggacctcgaaaaatacgtggaagataccaagatcgacctgtggtcctacaatgccgaactgctggtggccctggaaaaccagcacaccatcgacctgaccgacagcgagatgaacaagctgttcgaaaagaccaagaagcagctgcgcgagaacgccgaggatatgggcaacggctgctttaagatctaccacaagtgcgacaacgcctgcatcggctccatccggaacgagacatacgaccacaacgtgtacagagatgaggccctgaacaaccggttccagatcaaaggcgtggaactgaagtccggctacaaggactggatactgtggatcagcttcgccatgagctgctttctgctgtgtatcgctctgctgggcttcatcatgtgggcctgccagaaaggcaacatccggtgcaacatctgcatctgatga

SEQ ID NO: 98 - DNA, 2162 bp, synthetic construct
  source 1..2162: mol_type = other DNA
agaataaactagtattcttctggtccccacagactcagagagaacccgccaccatgaagaccatcattgccctgagctacatcctgtgcctggtgttcgcccagaagatccccggcaacgacaatagcaccgccacactgtgtctgggccatcacgctgtgcctaacggcaccatcgtgaaaaccatcaccaacgaccggatcgaagtgaccaacgccacagagctggtgcagaacagcagcatcggcgagatctgcgatagccctcaccagatcctggacggcggcaattgcacactgatcgatgccctgctgggcgaccctcagtgtgatggcttccagaacaaagaatgggacctgttcgtggaaagaagccgggccaacagcaactgctacccctacgatgtgcccgactacgccagcctgagatctctggtggcctctagcggcaccctggaattcaagaacgagagcttcaactggaccggcgtgaagcagaatggcaccagcagcgcctgtatcagaggcagcagctccagcttcttcagcagactgaactggctgacccacctgaactacacataccccgctctgaacgtgaccatgcctaacaacgagcagttcgacaagctgtacatctggggcgtgcaccatcctagcaccgacaaggatcagatcagcctgtttgcccagcctagcggcagaatcaccgtgtccactaagagaagccagcaggccgtgattcccaacatcggcagcagaccccggatcagagacatccccagccggatcagcatctactggacaatcgtgaagcccggcgacatcctgctgatcaacagcaccggcaatctgatcgcccctcggggctacttcaagatcagaagcggcaagagcagcatcatgcggagcgacgcccctatcggcaagtgcaagagcgagtgcatcaccccaaacggcagcatccccaacgacaagcccttccagaatgtgaaccggatcacctacggcgcctgtcctagatacgtgaaacagagcaccctgaagctggccaccggcatgagaaacgtgccagagaagcagaccagaggcatcttcggagccattgccggcttcatcgagaacggctgggaaggcatggtggacggatggtacggcttcagacaccagaacagcgaaggcagaggacaggccgctgacctgaaatctacacaggccgccatcgaccagatcaacggcaagctgaaccggctgatcggcaagaccaacgagaagttccaccagatcgagaaagagttcagcgaggtcgagggcagagtgcaggacctcgaaaaatacgtggaagataccaagatcgacctgtggtcctacaatgccgaactgctggtggccctggaaaaccagcacaccatcgacctgaccgacagcgagatgaacaagctgttcgaaaagaccaagaagcagctgcgcgagaacgccgaggatatgggcaacggctgctttaagatctaccacaagtgcgacaacgcctgcatcggctccatccggaacgagacatacgaccacaacgtgtacagagatgaggccctgaacaaccggttccagatcaaaggcgtggaactgaagtccggctacaaggactggatactgtggatcagcttcgccatgagctgctttctgctgtgtatcgctctgctgggcttcatcatgtgggcctgccagaaaggcaacatccggtgcaacatctgcatctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 99 - RNA, 2162 nt, synthetic construct
  source 1..2162: mol_type = other RNA
agaataaactagtattcttctggtccccacagactcagagagaacccgccaccatgaagaccatcattgccctgagctacatcctgtgcctggtgttcgcccagaagatccccggcaacgacaatagcaccgccacactgtgtctgggccatcacgctgtgcctaacggcaccatcgtgaaaaccatcaccaacgaccggatcgaagtgaccaacgccacagagctggtgcagaacagcagcatcggcgagatctgcgatagccctcaccagatcctggacggcggcaattgcacactgatcgatgccctgctgggcgaccctcagtgtgatggcttccagaacaaagaatgggacctgttcgtggaaagaagccgggccaacagcaactgctacccctacgatgtgcccgactacgccagcctgagatctctggtggcctctagcggcaccctggaattcaagaacgagagcttcaactggaccggcgtgaagcagaatggcaccagcagcgcctgtatcagaggcagcagctccagcttcttcagcagactgaactggctgacccacctgaactacacataccccgctctgaacgtgaccatgcctaacaacgagcagttcgacaagctgtacatctggggcgtgcaccatcctagcaccgacaaggatcagatcagcctgtttgcccagcctagcggcagaatcaccgtgtccactaagagaagccagcaggccgtgattcccaacatcggcagcagaccccggatcagagacatccccagccggatcagcatctactggacaatcgtgaagcccggcgacatcctgctgatcaacagcaccggcaatctgatcgcccctcggggctacttcaagatcagaagcggcaagagcagcatcatgcggagcgacgcccctatcggcaagtgcaagagcgagtgcatcaccccaaacggcagcatccccaacgacaagcccttccagaatgtgaaccggatcacctacggcgcctgtcctagatacgtgaaacagagcaccctgaagctggccaccggcatgagaaacgtgccagagaagcagaccagaggcatcttcggagccattgccggcttcatcgagaacggctgggaaggcatggtggacggatggtacggcttcagacaccagaacagcgaaggcagaggacaggccgctgacctgaaatctacacaggccgccatcgaccagatcaacggcaagctgaaccggctgatcggcaagaccaacgagaagttccaccagatcgagaaagagttcagcgaggtcgagggcagagtgcaggacctcgaaaaatacgtggaagataccaagatcgacctgtggtcctacaatgccgaactgctggtggccctggaaaaccagcacaccatcgacctgaccgacagcgagatgaacaagctgttcgaaaagaccaagaagcagctgcgcgagaacgccgaggatatgggcaacggctgctttaagatctaccacaagtgcgacaacgcctgcatcggctccatccggaacgagacatacgaccacaacgtgtacagagatgaggccctgaacaaccggttccagatcaaaggcgtggaactgaagtccggctacaaggactggatactgtggatcagcttcgccatgagctgctttctgctgtgtatcgctctgctgggcttcatcatgtgggcctgccagaaaggcaacatccggtgcaacatctgcatctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 100 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 101 - DNA, 1752 bp, synthetic construct
  source 1..1752: mol_type = other DNA
atgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagccacttcgccaacctgaagggcaccgagacaagaggcaagctgtgccccaagtgcctgaactgcaccgatctggatgtggccctgggcagacctaagtgtaccggcaagatccctagcgccagagtgtccatcctgcacgaagtgcggcctgtgaccagcggctgctttcccattatgcacgaccggaccaagatcagacagctgcccaatctgctgcggggctatgaacatgtgcggctgagcacccacaacgtgatcaacgccgaagatgcccctggcagaccatacgagatcggcacatctggctcttgccccaacattaccaacggcaacggcttcttcgccaccatggcttgggccgtgcctaagaacaagaccgccaccaatccactgaccatcgaggtgccctacatctgtaccgaaggcgaggaccagatcaccgtgtggggcttccacagcgacaacgagacacagatggccaaactgtacggcgacagcaagccccagaagttcaccagctctgccaacggcgtgaccacacactacgtgtcccagatcggcggcttccccaatcagacagaagatggcggactgccccagagcggaagaatcgtggtggactacatggtgcagaagtccggcaagaccggcacaatcacataccagcggggaatcctgctgcctcagaaagtttggtgcgccagcggccggtccaaagtgatcaaaggatcactgcctctgatcggcgaggccgattgtctgcacgagaaatacggcggcctgaacaagagcaagccctactacacaggcgagcacgccaaggccatcggcaactgtcctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggcccaagcgccgtggaaatcggcaatggctgcttcgagacaaagcacaagtgcaaccagacctgcctggacagaattgccgccggaacatttgacgccggcgagtttagcctgcctaccttcgacagcctgaacatcacagccgccagcctgaatgacgacggcctggacaatcacaccatcctgctgtactactccaccgccgcatcttctctggccgtgacactgatgatcgccatctttgtggtgtacatggtgtccagagacaacgtgtcctgcagcatctgcctgtagtga

SEQ ID NO: 102 - RNA, 1752 nt, synthetic construct
  source 1..1752: mol_type = other RNA
atgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagccacttcgccaacctgaagggcaccgagacaagaggcaagctgtgccccaagtgcctgaactgcaccgatctggatgtggccctgggcagacctaagtgtaccggcaagatccctagcgccagagtgtccatcctgcacgaagtgcggcctgtgaccagcggctgctttcccattatgcacgaccggaccaagatcagacagctgcccaatctgctgcggggctatgaacatgtgcggctgagcacccacaacgtgatcaacgccgaagatgcccctggcagaccatacgagatcggcacatctggctcttgccccaacattaccaacggcaacggcttcttcgccaccatggcttgggccgtgcctaagaacaagaccgccaccaatccactgaccatcgaggtgccctacatctgtaccgaaggcgaggaccagatcaccgtgtggggcttccacagcgacaacgagacacagatggccaaactgtacggcgacagcaagccccagaagttcaccagctctgccaacggcgtgaccacacactacgtgtcccagatcggcggcttccccaatcagacagaagatggcggactgccccagagcggaagaatcgtggtggactacatggtgcagaagtccggcaagaccggcacaatcacataccagcggggaatcctgctgcctcagaaagtttggtgcgccagcggccggtccaaagtgatcaaaggatcactgcctctgatcggcgaggccgattgtctgcacgagaaatacggcggcctgaacaagagcaagccctactacacaggcgagcacgccaaggccatcggcaactgtcctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggcccaagcgccgtggaaatcggcaatggctgcttcgagacaaagcacaagtgcaaccagacctgcctggacagaattgccgccggaacatttgacgccggcgagtttagcctgcctaccttcgacagcctgaacatcacagccgccagcctgaatgacgacggcctggacaatcacaccatcctgctgtactactccaccgccgcatcttctctggccgtgacactgatgatcgccatctttgtggtgtacatggtgtccagagacaacgtgtcctgcagcatctgcctgtagtga

SEQ ID NO: 103 - DNA, 2210 bp, synthetic construct
  source 1..2210: mol_type = other DNA
agaataaactagtattcttctggtccccacagactcagagagaacccgccaccatgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagccacttcgccaacctgaagggcaccgagacaagaggcaagctgtgccccaagtgcctgaactgcaccgatctggatgtggccctgggcagacctaagtgtaccggcaagatccctagcgccagagtgtccatcctgcacgaagtgcggcctgtgaccagcggctgctttcccattatgcacgaccggaccaagatcagacagctgcccaatctgctgcggggctatgaacatgtgcggctgagcacccacaacgtgatcaacgccgaagatgcccctggcagaccatacgagatcggcacatctggctcttgccccaacattaccaacggcaacggcttcttcgccaccatggcttgggccgtgcctaagaacaagaccgccaccaatccactgaccatcgaggtgccctacatctgtaccgaaggcgaggaccagatcaccgtgtggggcttccacagcgacaacgagacacagatggccaaactgtacggcgacagcaagccccagaagttcaccagctctgccaacggcgtgaccacacactacgtgtcccagatcggcggcttccccaatcagacagaagatggcggactgccccagagcggaagaatcgtggtggactacatggtgcagaagtccggcaagaccggcacaatcacataccagcggggaatcctgctgcctcagaaagtttggtgcgccagcggccggtccaaagtgatcaaaggatcactgcctctgatcggcgaggccgattgtctgcacgagaaatacggcggcctgaacaagagcaagccctactacacaggcgagcacgccaaggccatcggcaactgtcctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggcccaagcgccgtggaaatcggcaatggctgcttcgagacaaagcacaagtgcaaccagacctgcctggacagaattgccgccggaacatttgacgccggcgagtttagcctgcctaccttcgacagcctgaacatcacagccgccagcctgaatgacgacggcctggacaatcacaccatcctgctgtactactccaccgccgcatcttctctggccgtgacactgatgatcgccatctttgtggtgtacatggtgtccagagacaacgtgtcctgcagcatctgcctgtagtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 104 - RNA, 2210 nt, synthetic construct
  source 1..2210: mol_type = other RNA
agaataaactagtattcttctggtccccacagactcagagagaacccgccaccatgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagccacttcgccaacctgaagggcaccgagacaagaggcaagctgtgccccaagtgcctgaactgcaccgatctggatgtggccctgggcagacctaagtgtaccggcaagatccctagcgccagagtgtccatcctgcacgaagtgcggcctgtgaccagcggctgctttcccattatgcacgaccggaccaagatcagacagctgcccaatctgctgcggggctatgaacatgtgcggctgagcacccacaacgtgatcaacgccgaagatgcccctggcagaccatacgagatcggcacatctggctcttgccccaacattaccaacggcaacggcttcttcgccaccatggcttgggccgtgcctaagaacaagaccgccaccaatccactgaccatcgaggtgccctacatctgtaccgaaggcgaggaccagatcaccgtgtggggcttccacagcgacaacgagacacagatggccaaactgtacggcgacagcaagccccagaagttcaccagctctgccaacggcgtgaccacacactacgtgtcccagatcggcggcttccccaatcagacagaagatggcggactgccccagagcggaagaatcgtggtggactacatggtgcagaagtccggcaagaccggcacaatcacataccagcggggaatcctgctgcctcagaaagtttggtgcgccagcggccggtccaaagtgatcaaaggatcactgcctctgatcggcgaggccgattgtctgcacgagaaatacggcggcctgaacaagagcaagccctactacacaggcgagcacgccaaggccatcggcaactgtcctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggcccaagcgccgtggaaatcggcaatggctgcttcgagacaaagcacaagtgcaaccagacctgcctggacagaattgccgccggaacatttgacgccggcgagtttagcctgcctaccttcgacagcctgaacatcacagccgccagcctgaatgacgacggcctggacaatcacaccatcctgctgtactactccaccgccgcatcttctctggccgtgacactgatgatcgccatctttgtggtgtacatggtgtccagagacaacgtgtcctgcagcatctgcctgtagtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 105 - protein, 584 aa, synthetic construct
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 106 - DNA, 1758 bp, synthetic construct
  source 1..1758: mol_type = other DNA
atgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagctacttcgccaacctgaagggcaccagaaccagaggcaagctgtgccccgattgcctgaactgcaccgatctggatgtggccctgggcagacctatgtgcgtgggaacaacacctagcgccaaggccagcatcctgcatgaagtgcggcctgtgaccagcggctgcttccctattatgcacgaccggaccaagatcagacagctgcccaatctgctgcggggctacgagaagatcaggctgagcacccagaacgtgatcgacgccgaaaaagctcctggcggcccttacagactgggcacatctggctcttgccccaacgctacaagcaagatcggcttcttcgccaccatggcctgggccgtgcctaaggacaactacaagaacgccaccaatcctctgaccgtggaagtgccctacatctgtaccgaaggcgaggaccagatcaccgtgtggggcttccacagcgacaacaagacccagatgaagtccctgtacggcgacagcaaccctcagaagtttaccagcagcgccaacggcgtgaccacacactatgtgtcccagatcggcgacttccccgaccagacagaagatggcggactgcctcagagcggcagaatcgtggtggactacatgatgcagaagcccggcaagaccggcaccatcgtgtatcagagaggcgtcctgctgccacagaaagtttggtgcgccagcggccggtccaaagtgatcaaaggatcactgcctctgatcggcgaggccgactgtctgcacgaagaatatggcggcctgaacaagagcaagccctactacacaggcaagcacgccaaagccatcggcaactgccctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggacccagcgccgtggatatcggcaatggctgcttcgagacaaagcacaagtgcaaccagacctgcctggacagaattgccgccggaacctttaacgccggcgagtttagcctgcctaccttcgacagcctgaacatcacagccgccagcctgaatgacgacggcctggacaatcacaccatcctgctgtactactccaccgccgcatcttctctggccgtgacactgatgctggctatcttcatcgtgtacatggtgtccagagacaacgtgtcctgcagcatctgcctgtgatga

SEQ ID NO: 107 - RNA, 1758 nt, synthetic construct
  source 1..1758: mol_type = other RNA
atgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagctacttcgccaacctgaagggcaccagaaccagaggcaagctgtgccccgattgcctgaactgcaccgatctggatgtggccctgggcagacctatgtgcgtgggaacaacacctagcgccaaggccagcatcctgcatgaagtgcggcctgtgaccagcggctgcttccctattatgcacgaccggaccaagatcagacagctgcccaatctgctgcggggctacgagaagatcaggctgagcacccagaacgtgatcgacgccgaaaaagctcctggcggcccttacagactgggcacatctggctcttgccccaacgctacaagcaagatcggcttcttcgccaccatggcctgggccgtgcctaaggacaactacaagaacgccaccaatcctctgaccgtggaagtgccctacatctgtaccgaaggcgaggaccagatcaccgtgtggggcttccacagcgacaacaagacccagatgaagtccctgtacggcgacagcaaccctcagaagtttaccagcagcgccaacggcgtgaccacacactatgtgtcccagatcggcgacttccccgaccagacagaagatggcggactgcctcagagcggcagaatcgtggtggactacatgatgcagaagcccggcaagaccggcaccatcgtgtatcagagaggcgtcctgctgccacagaaagtttggtgcgccagcggccggtccaaagtgatcaaaggatcactgcctctgatcggcgaggccgactgtctgcacgaagaatatggcggcctgaacaagagcaagccctactacacaggcaagcacgccaaagccatcggcaactgccctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggacccagcgccgtggatatcggcaatggctgcttcgagacaaagcacaagtgcaaccagacctgcctggacagaattgccgccggaacctttaacgccggcgagtttagcctgcctaccttcgacagcctgaacatcacagccgccagcctgaatgacgacggcctggacaatcacaccatcctgctgtactactccaccgccgcatcttctctggccgtgacactgatgctggctatcttcatcgtgtacatggtgtccagagacaacgtgtcctgcagcatctgcctgtgatga

SEQ ID NO: 108 - DNA, 2216 bp, synthetic construct
  source 1..2216: mol_type = other DNA
agaataaactagtattcttctggtccccacagactcagagagaacccgccaccatgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagctacttcgccaacctgaagggcaccagaaccagaggcaagctgtgccccgattgcctgaactgcaccgatctggatgtggccctgggcagacctatgtgcgtgggaacaacacctagcgccaaggccagcatcctgcatgaagtgcggcctgtgaccagcggctgcttccctattatgcacgaccggaccaagatcagacagctgcccaatctgctgcggggctacgagaagatcaggctgagcacccagaacgtgatcgacgccgaaaaagctcctggcggcccttacagactgggcacatctggctcttgccccaacgctacaagcaagatcggcttcttcgccaccatggcctgggccgtgcctaaggacaactacaagaacgccaccaatcctctgaccgtggaagtgccctacatctgtaccgaaggcgaggaccagatcaccgtgtggggcttccacagcgacaacaagacccagatgaagtccctgtacggcgacagcaaccctcagaagtttaccagcagcgccaacggcgtgaccacacactatgtgtcccagatcggcgacttccccgaccagacagaagatggcggactgcctcagagcggcagaatcgtggtggactacatgatgcagaagcccggcaagaccggcaccatcgtgtatcagagaggcgtcctgctgccacagaaagtttggtgcgccagcggccggtccaaagtgatcaaaggatcactgcctctgatcggcgaggccgactgtctgcacgaagaatatggcggcctgaacaagagcaagccctactacacaggcaagcacgccaaagccatcggcaactgccctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggacccagcgccgtggatatcggcaatggctgcttcgagacaaagcacaagtgcaaccagacctgcctggacagaattgccgccggaacctttaacgccggcgagtttagcctgcctaccttcgacagcctgaacatcacagccgccagcctgaatgacgacggcctggacaatcacaccatcctgctgtactactccaccgccgcatcttctctggccgtgacactgatgctggctatcttcatcgtgtacatggtgtccagagacaacgtgtcctgcagcatctgcctgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 109 - RNA, 2216 nt, synthetic construct
  source 1..2216: mol_type = other RNA
agaataaactagtattcttctggtccccacagactcagagagaacccgccaccatgaaggccatcatcgtgctgctgatggtggtcaccagcaacgccgacagaatctgcaccggcatcaccagcagcaacagccctcacgtggtcaagacagccacacagggcgaagtgaatgtgaccggcgtgatccctctgaccaccacacctaccaagagctacttcgccaacctgaagggcaccagaaccagaggcaagctgtgccccgattgcctgaactgcaccgatctggatgtggccctgggcagacctatgtgcgtgggaacaacacctagcgccaaggccagcatcctgcatgaagtgcggcctgtgaccagcggctgcttccctattatgcacgaccggaccaagatcagacagctgcccaatctgctgcggggctacgagaagatcaggctgagcacccagaacgtgatcgacgccgaaaaagctcctggcggcccttacagactgggcacatctggctcttgccccaacgctacaagcaagatcggcttcttcgccaccatggcctgggccgtgcctaaggacaactacaagaacgccaccaatcctctgaccgtggaagtgccctacatctgtaccgaaggcgaggaccagatcaccgtgtggggcttccacagcgacaacaagacccagatgaagtccctgtacggcgacagcaaccctcagaagtttaccagcagcgccaacggcgtgaccacacactatgtgtcccagatcggcgacttccccgaccagacagaagatggcggactgcctcagagcggcagaatcgtggtggactacatgatgcagaagcccggcaagaccggcaccatcgtgtatcagagaggcgtcctgctgccacagaaagtttggtgcgccagcggccggtccaaagtgatcaaaggatcactgcctctgatcggcgaggccgactgtctgcacgaagaatatggcggcctgaacaagagcaagccctactacacaggcaagcacgccaaagccatcggcaactgccctatctgggtcaagacccctctgaagctggccaacggcaccaagtatagacctccagccaagctgctgaaagagcggggcttctttggagctatcgccggctttcttgaaggcggctgggagggaatgattgccggctggcatggctacacatctcatggcgcacatggcgtggcagtggccgctgatctgaagtctacacaagaggccatcaacaagatcaccaagaacctgaacagcctgagcgagctggaagtgaagaacctgcagagactgtccggcgccatggacgagctgcacaacgagatcctggaactggacgagaaggtggacgacctgagagccgataccatctccagccagattgagctggcagtgctgctgtccaacgagggcatcatcaacagcgaggacgagcatctgctggccctggaacggaagctgaagaagatgctgggacccagcgccgtggatatcggcaatggctgcttcgagacaaagcacaagtgcaaccagacctgcctggacagaattgccgccggaacctttaacgccggcgagtttagcctgcctaccttcgacagcctgaacatcacagccgccagcctgaatgacgacggcctggacaatcacaccatcctgctgtactactccaccgccgcatcttctctggccgtgacactgatgctggctatcttcatcgtgtacatggtgtccagagacaacgtgtcctgcagcatctgcctgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 110 - protein, 27 aa, Human alphaherpesvirus 1
MGGAAARLGAVILFVVIVGLHGVRSKY

SEQ ID NO: 111 - protein, 25 aa, Human alphaherpesvirus 2
MGRLTSGVGTAALLVVAVGLRVVCA

SEQ ID NO: 112 - protein, 28 aa, Human alphaherpesvirus 2
MGRLTSGVGTAALLVVAVGLRVVCAKYA

SEQ ID NO: 113 - protein, 19 aa, Severe acute respiratory syndrome coronavirus 2
MFVFLVLLPLVSSQCVNLT

SEQ ID NO: 114 - protein, 21 aa, synthetic construct
MDWIWRILFLVGAATGAHSQM

SEQ ID NO: 115 - protein, 20 aa, Homo sapiens
METPAQLLFLLLLWLPDTTG

SEQ ID NO: 116 - protein, 18 aa, synthetic construct
MDWTWILFLVAAATRVHS

SEQ ID NO: 117 - protein, 24 aa, Japanese encephalitis virus
MLGSNSGQRVVFTILLLLVAPAYS

SEQ ID NO: 118 - protein, 17 aa, synthetic construct
MKCLLYLAFLFIGVNCA

SEQ ID NO: 119 - protein, 18 aa, synthetic construct
MDWTWILFLVAAATRVHS

SEQ ID NO: 120 - protein, 19 aa, synthetic construct
ETPAQLLFLLLLWLPDTTG

SEQ ID NO: 121 - protein, 24 aa, synthetic construct
MLGSNSGQRVVFTILLLLVAPAYS

SEQ ID NO: 122 - protein, 17 aa, synthetic construct
MKCLLYLAFLFIGVNCA

SEQ ID NO: 123 - protein, 15 aa, synthetic construct
MWLVSLAIVTACAGA

SEQ ID NO: 124 - protein, 15 aa, synthetic construct
MFVFLVLLPLVSSQC

SEQ ID NO: 125 - DNA, 81 bp, Human alphaherpesvirus 1
  source 1..81: mol_type = genomic DNA
atggggggggctgccgccaggttgggggccgtgattttgtttgtcgtcatagtgggcctccatggggtccgcagcaaatat

SEQ ID NO: 126 - DNA, 81 bp, synthetic construct
  source 1..81: mol_type = other DNA
atgggaggagccgccgccagactgggagccgtgatcctgttcgtggtgatcgtgggactgcatggagtgagaagcaagtac

SEQ ID NO: 127 - DNA, 57 bp, Severe acute respiratory syndrome coronavirus 2
  source 1..57: mol_type = genomic DNA
atgtttgtgtttcttgtgctgctgcctcttgtgtcttctcagtgtgtgaatttgaca

SEQ ID NO: 128 - DNA, 63 bp, synthetic construct
  source 1..63: mol_type = other DNA
atggattggatttggagaatcctgttcctcgtgggagccgctacaggagcccactcccagatg

SEQ ID NO: 129 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 130 - RNA, 3813 nt, synthetic construct
  source 1..3813: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtggatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 131 - DNA, 3813 bp, synthetic construct
  source 1..3813: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtggatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 132 - RNA, 4270 nt, synthetic construct
  source 1..4270: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtggatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 133 - DNA, 4270 bp, synthetic construct
  source 1..4270: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtggatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 134 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLVGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNRPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 135 - RNA, 3813 nt, synthetic construct
  source 1..3813: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggtgggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtggatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaggccttgtaacggcgtggcaggccccaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 136 - DNA, 3813 bp, synthetic construct
  source 1..3813: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggtgggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtggatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaggccttgtaacggcgtggcaggccccaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 137 - RNA, 4270 nt, synthetic construct
  source 1..4270: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggtgggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtggatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaggccttgtaacggcgtggcaggccccaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 138 - DNA, 4270 bp, synthetic construct
  source 1..4270: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggtgggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtggatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaggccttgtaacggcgtggcaggccccaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 139 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHASATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 140 - RNA, 3813 nt, synthetic construct
  source 1..3813: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcggcagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcctcagccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 141 - DNA, 3813 bp, synthetic construct
  source 1..3813: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcggcagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcctcagccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 142 - RNA, 4270 nt, synthetic construct
  source 1..4270: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcggcagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcctcagccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 143 - DNA, 4270 bp, synthetic construct
  source 1..4270: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcggcagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcctcagccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 144 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEVNFKNLREFVFKNIDGYFKIYSKHTPINLERDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHASATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 145 - RNA, 3813 nt, synthetic construct
  source 1..3813: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggaggtgaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcggcagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcctcagccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 146 - DNA, 3813 bp, synthetic construct
  source 1..3813: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggaggtgaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcggcagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcctcagccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 147 - RNA, 4270 nt, synthetic construct
  source 1..4270: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggaggtgaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcggcagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcctcagccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 148 - DNA, 4270 bp, synthetic construct
  source 1..4270: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggaggtgaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcggcagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggccccaactgctacagcccactgcagtcctacggctttaggcccacatacggcgtgggccaccagccctacagagtggtggtgctgagcttcgaactgctgcatgcctcagccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 149 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 150 - RNA, 3816 nt, synthetic construct
  source 1..3816: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacgagaacaacaagagcaggatggaaagcgagctccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctgtgaacctcggccgggatctgcctcagggcttctctgctctggaacccctggtggatctgcccatcggcatcaacatcacccggtttcagacactgctggccctgcacagaagctacctgacacctggcgatagcagcagcagctggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaagctgaagcccttcgagcgggacatctccaccgagatctatcaggccggcaacaagccttgtaacggcgtggcaggcttcaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 151 - DNA, 3816 bp, synthetic construct
  source 1..3816: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacgagaacaacaagagcaggatggaaagcgagctccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctgtgaacctcggccgggatctgcctcagggcttctctgctctggaacccctggtggatctgcccatcggcatcaacatcacccggtttcagacactgctggccctgcacagaagctacctgacacctggcgatagcagcagcagctggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaagctgaagcccttcgagcgggacatctccaccgagatctatcaggccggcaacaagccttgtaacggcgtggcaggcttcaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 152 - RNA, 4273 nt, synthetic construct
  source 1..4273: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacgagaacaacaagagcaggatggaaagcgagctccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctgtgaacctcggccgggatctgcctcagggcttctctgctctggaacccctggtggatctgcccatcggcatcaacatcacccggtttcagacactgctggccctgcacagaagctacctgacacctggcgatagcagcagcagctggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaagctgaagcccttcgagcgggacatctccaccgagatctatcaggccggcaacaagccttgtaacggcgtggcaggcttcaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 153 - DNA, 4273 bp, synthetic construct
  source 1..4273: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacgagaacaacaagagcaggatggaaagcgagctccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctgtgaacctcggccgggatctgcctcagggcttctctgctctggaacccctggtggatctgcccatcggcatcaacatcacccggtttcagacactgctggccctgcacagaagctacctgacacctggcgatagcagcagcagctggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccagattcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaagctgaagcccttcgagcgggacatctccaccgagatctatcaggccggcaacaagccttgtaacggcgtggcaggcttcaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 154 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 155 - RNA, 3816 nt, synthetic construct
  source 1..3816: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacgagaacaacaagagcaggatggaaagcgagctccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctgtgaacctcggccgggatctgcctcagggcttctctgctctggaacccctggtggatctgcccatcggcatcaacatcacccggtttcagacactgctggccctgcacagaagctacctgacacctggcgatagcagcagcagctggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaagctgaagcccttcgagcgggacatctccaccgagatctatcaggccggcaacaagccttgtaacggcgtggcaggcagcaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcaacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 156 - DNA, 3816 bp, synthetic construct
  source 1..3816: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacgagaacaacaagagcaggatggaaagcgagctccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctgtgaacctcggccgggatctgcctcagggcttctctgctctggaacccctggtggatctgcccatcggcatcaacatcacccggtttcagacactgctggccctgcacagaagctacctgacacctggcgatagcagcagcagctggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaagctgaagcccttcgagcgggacatctccaccgagatctatcaggccggcaacaagccttgtaacggcgtggcaggcagcaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcaacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 157 - RNA, 4273 nt, synthetic construct
  source 1..4273: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacgagaacaacaagagcaggatggaaagcgagctccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctgtgaacctcggccgggatctgcctcagggcttctctgctctggaacccctggtggatctgcccatcggcatcaacatcacccggtttcagacactgctggccctgcacagaagctacctgacacctggcgatagcagcagcagctggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaagctgaagcccttcgagcgggacatctccaccgagatctatcaggccggcaacaagccttgtaacggcgtggcaggcagcaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcaacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 158 - DNA, 4273 bp, synthetic construct
  source 1..4273: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacgagaacaacaagagcaggatggaaagcgagctccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctgtgaacctcggccgggatctgcctcagggcttctctgctctggaacccctggtggatctgcccatcggcatcaacatcacccggtttcagacactgctggccctgcacagaagctacctgacacctggcgatagcagcagcagctggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaagctgaagcccttcgagcgggacatctccaccgagatctatcaggccggcaacaagccttgtaacggcgtggcaggcagcaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcaacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 159 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVRQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 160 - RNA, 3810 nt, synthetic construct
  source 1..3810: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgcggcagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 161 - DNA, 3810 bp, synthetic construct
  source 1..3810: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgcggcagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 162 - RNA, 4267 nt, synthetic construct
  source 1..4267: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgcggcagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 163 - DNA, 4267 bp, synthetic construct
  source 1..4267: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgcggcagattgcccctggacagacaggcaacatcgccgactacaactacaagctgcccgacgacttcaccggctgtgtgattgcctggaacagcaacaagctggactccaaagtcggcggcaactacaattacaggtaccggctgttccggaagtccaatctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 164 - protein, 1269 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 165 - RNA, 3813 nt, synthetic construct
  source 1..3813: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggcagcaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 166 - DNA, 3813 bp, synthetic construct
  source 1..3813: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggcagcaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 167 - RNA, 4270 nt, synthetic construct
  source 1..4270: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggcagcaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 168 - DNA, 4270 bp, synthetic construct
  source 1..4270: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgccctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctaccagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgtttaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggcaggcagcaactgctacagcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 169 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 170 - RNA, 3810 nt, synthetic construct
  source 1..3810: mol_type = other RNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaccgtcggcggcaactacaattacaggtaccggctgttccggaagtccaagctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 171 - DNA, 3810 bp, synthetic construct
  source 1..3810: mol_type = other DNA
atgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaccgtcggcggcaactacaattacaggtaccggctgttccggaagtccaagctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatga

SEQ ID NO: 172 - RNA, 4267 nt, synthetic construct
  source 1..4267: mol_type = other RNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaccgtcggcggcaactacaattacaggtaccggctgttccggaagtccaagctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 173 - DNA, 4267 bp, synthetic construct
  source 1..4267: mol_type = other DNA
agaataaactagtattcttctggtccccacagactcagagagaacccgccaccatgttcgtgttcctggtgctgctgcctctggtgtccagccagtgtgtgaacctgatcaccagaacacagtcatacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccccttcttcgcattcaagtgctacggcgtgtcccctaccaagctgaacgacctgtgcttcacaaacgtgtacgccgacagcttcgtgatccggggaaacgaagtgtcacagattgcccctggacagacaggcaacatcgccgactacaactacaagctgcccgacgacttcaccggctgtgtgattgcctggaacagcaacaagctggactccaccgtcggcggcaactacaattacaggtaccggctgttccggaagtccaagctgaagcccttcgagcgggacatctccaccgagatctatcaggccggcaacaagccttgtaacggcgtggcaggcgtgaactgctacttcccactgcagtcctacggctttaggcccacatacggcgtgggccaccagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggaatctgcgccagctaccagacacagacaaagagccaccggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaaaagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccacaatgcccaggcactgaacaccctggtcaagcagctgtcctccaagttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 174 - RNA, 30 nt, synthetic construct
  source 1..30: mol_type = other RNA
aaaaaaaaaaaaaaaaaaaaaaaaaaaaaa

SEQ ID NO: 175 - RNA, 70 nt, synthetic construct
  source 1..70: mol_type = other RNA
aaaaaaaaaaaaaaaaaaaaaaaaaaaaaaaaaaaaaaaaaaaaaaaaaaaaaaaaaaaaaaaaaaaaaa

SEQ ID NO: 176 - RNA, 11 nt, synthetic construct
  source 1..11: mol_type = other RNA
ctngagctagc

SEQ ID NO: 177 - RNA, 33 nt, synthetic construct
  source 1..33: mol_type = other RNA
gagacctggtccagagtcgctagccgcgtcgct

SEQ ID NO: 178 - protein, 4 aa, synthetic construct
RRAR

SEQ ID NO: 179 - RNA, 12 nt, synthetic construct
  source 1..12: mol_type = other RNA
cggagagccaga

SEQ ID NO: 180 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0144861 A1
(43) Pub. Date: May 28, 2026

(54) COMBINATION VACCINES AGAINST CORONAVIRUS INFECTION, INFLUENZA INFECTION, AND/OR RSV INFECTION

(71) Applicants: BioNTech SE, Mainz (DE); Pfizer Inc., New York, NY (US)

(72) Inventors: Ugur Sahin, Mainz (DE); Nadine Salisch, Treuchtlingen (DE); Federico Mensa, Glencoe, IL (US); Nicholas Randolph Everard Kitchin, Sandwich, Kent (GB); Annaliesa Sybil Anderson, Upper Saddle River, NJ (US); Kena Anne Swanson, Pearl River, NY (US); Advait Vijay Badkar, Westford, MA (US); Ramin Darvari, Lexington, MA (US); Mark Duda, Lynn, MA (US); Alejandra Clarisa Gurtman, Englewood Cliffs, NJ (US); Christina Van Geen Hoven, Boxford, MA (US); Pirada Suphaphiphat Allen, Congers, NY (US)

(21) Appl. No.: 19/121,711

(22) PCT Filed: Oct. 17, 2023
(86) PCT No.: PCT/US2023/077086

§ 371 Date: Apr. 16, 2025

Related U.S. Application Data
(60) Provisional application No. 63/416,933, filed on Oct. 17, 2022.
(60) Provisional application No. 63/431,615, filed on Dec. 9, 2022.
(60) Provisional application No. 63/437,967, filed on Jan. 9, 2023.
(60) Provisional application No. 63/465,516, filed on May 10, 2023.
(60) Provisional application No. 63/469,473, filed on May 29, 2023.

(51) Int. Cl. A61K 39/215; A61K 9/1271; A61K 9/51; A61K 39/00; A61K 39/09; A61K 39/145; A61K 39/155; A61M 5/19; A61P 31/16; B82Y 5/00; C07K 14/005; C12N 7/00

(52) U.S. Cl. CPC A61K 39/215; A61K 9/1271; A61K 9/5123; A61K 39/092; A61K 39/145; A61K 39/155; A61M 5/19; A61P 31/16; C07K 14/005; C12N 7/00; A61K 2039/53; A61K 2039/54; A61K 2039/545; A61K 2039/55555; A61K 2039/70; B82Y 5/00; C12N 2760/16122; C12N 2760/16134; C12N 2760/16222; C12N 2760/16234; C12N 2760/18522; C12N 2760/18534; C12N 2770/20022; C12N 2770/20034; C12N 2830/50

(57) ABSTRACT

This disclosure relates to the field of RNA to prevent or treat multiple infectious agents. In particular, the present disclosure relates to methods and agents for vaccination against coronavirus infection, influenza infection, and/or RSV infection and inducing effective coronavirus, influenza virus, and/or RSV antigen-specific immune responses such as antibody and/or T cell responses. Specifically, in one embodiment, the present disclosure relates to methods comprising administering to a subject (i) a bivalent RNA vaccine encoding peptides or proteins comprising epitopes of SARS-CoV-2 spike proteins (S proteins) and (ii) a tetravalent RNA vaccine encoding peptides or proteins comprising epitopes of hemagglutinin (HA), for inducing an immune response against coronavirus S proteins, in particular S proteins of SARS-CoV-2, and influenza proteins, in particular HA proteins of type A and type B influenza viruses, in the subject.

### PRIORITY CLAIM

This application is the National Stage Application of International Application No. PCT/US23/77086, filed Oct. 17, 2023, which claims the benefit of each of the following applications, the disclosure of each of which is hereby incorporated by reference in its entirety: U.S. provisional application No. 63/416,933, filed Oct. 17, 2022; U.S. provisional application No. 63/431,615, filed Dec. 9, 2022; U.S. provisional application No. 63/437,967, filed Jan. 9, 2023; U.S. provisional application No. 63/465,516, filed May 10, 2023; and U.S. provisional application No. 63/469,473, filed May 29, 2023.

### SEQUENCE LISTING

The instant application contains a Sequence Listing which has been submitted electronically in XML file format and is hereby incorporated by reference in its entirety. Said XML copy, created on Oct. 12, 2023, is named “2013237-0765_SL.xml” and is 548,288 bytes in size.

### BACKGROUND

Viral infections represent a major threat to human health and well-being. For example, coronaviruses are a group of RNA viruses that cause respiratory tract infections that can range from mild to lethal. Mild illnesses in humans include some cases of the common cold (which is also caused by other viruses, predominantly rhinoviruses), while more lethal varieties can cause SARS, MERS and COVID-19.

Influenza, commonly known as “the flu”, is an infectious disease caused by influenza viruses, a family of negative-sense RNA viruses. Symptoms range from mild to severe and often include fever, runny nose, sore throat, muscle pain, headache, coughing, and fatigue. In a typical year, 5-15% of the population contracts influenza, with 3-5 million severe cases annually and up to 650,000 respiratory-related deaths globally each year.

### SUMMARY

The present disclosure relates to concurrent delivery of multiple antigenic polypeptides to subjects (e.g., human subjects) and related technologies (e.g., methods) for preventing and/or treating multiple infectious agents. In some embodiments, such infectious agents may include, but are not limited to infectious bacterial agents and viral agents.

In some embodiments, the present disclosure provides pharmaceutical compositions (e.g., immunogenic compositions, e.g., vaccines) that achieve such concurrent delivery of multiple antigenic polypeptides. In some embodiments, the present disclosure provides certain combination compositions that are particularly useful in effective vaccination. In some embodiments, such combination compositions comprise a plurality of RNAs encoding antigenic polypeptides of (e.g., that induce or promote immunity to) at least two different infectious diseases (e.g., in some embodiments infectious respiratory diseases).

In some embodiments, an antigenic polypeptide as described herein is a polypeptide comprising at least one antigen epitope. In some embodiments, an antigenic polypeptide is a full-length antigen. In some embodiments, an antigenic polypeptide is an immunogenic fragment of a full-length antigen. In some embodiments, an antigenic polypeptide may comprise one or more modifications (e.g., in some embodiments substitutions) relative to a full-length or an immunogenic fragment, e.g., as found in the relevant infectious agent. In some embodiments, an antigen epitope or antigenic polypeptide is cross-reactive with (e.g., induces and/or promotes an immune response to) a corresponding epitope or polypeptide in an infectious agent (e.g., in a virus such as a respiratory virus).

The present disclosure, among other things, provides a recognition that annual vaccine programs against certain infectious diseases (e.g., influenza, respiratory syncytial virus disease, and/or coronavirus disease) can be conducted at a similar time of the year and that existing vaccine vectors and traditional technologies may limit time to manufacture from selection of seasonal strains. For example, influenza and coronavirus vaccines are currently available, but the present disclosure identifies the source of a problem with current vaccination programs, and, moreover, provides improved vaccination technologies, including particular vaccine compositions and strategies.

Due to observed waning in neutralizing antibody titers over time after a primary series of COVID-19 vaccines, booster dosing is recommended to restore or maintain robust immunity and disease protection. Furthermore, the continued evolution of new SARS-CoV-2 variants warrants vaccine strain changes. Influenza vaccines are also regularly updated (typically annually). Existing influenza vaccines have limitations, including, for example time to manufacture from selection of seasonal strains, complexity of manufacturing, and limited effectiveness. The present disclosure, among other things, provides a recognition that combination RNA vaccines against certain infectious diseases (e.g., infectious respiratory diseases) may be beneficial to address certain limitations of the existing individual vaccines against certain infectious diseases. For example, in one aspect, the present disclosure provides a recognition that combination RNA vaccines may provide certain benefits, including, e.g., but are not limited to, potential for accelerated manufacturing, for example, with no reassortment step, and/or reduced probability of vaccine being mismatched with seasonal circulating strains, and/or improved efficacy relative to currently licensed vaccines through induction of strong T cells responses (e.g., CD4+ and/or CD8+ T cell responses).

In one aspect, the present disclosure relates to technologies (e.g., compositions and methods) for vaccination against coronavirus and influenza virus infection or disease and inducing effective coronavirus and influenza virus antigen-specific immune responses such as antibody and/or T cell responses. In some embodiments, such technologies based on RNA technologies are, in particular, useful for the prevention or treatment of coronavirus and influenza virus infections and/or disease. Administration of RNA disclosed herein to a subject can protect the subject against coronavirus infection and/or influenza virus infection (e.g., reducing probability that an exposure will result in established infection and/or in disease).

In some embodiments, the present disclosure provides technologies (e.g., composition and/or methods) for protection against coronavirus and influenza virus infection by administering a subject RNA encoding a coronavirus antigenic polypeptide and RNA encoding an influenza antigenic polypeptide. Specifically, in one embodiment, the present disclosure relates to methods comprising administering to a subject RNA encoding a coronavirus a peptide or protein comprising an epitope of SARS-CoV-2 spike protein (S protein), in particular S protein of SARS-CoV-2, and RNA encoding a peptide or protein comprising an epitope of a Hemagglutinin (HA) protein, for inducing an immune response against coronavirus S protein and an immune response against Hemagglutinin, i.e., vaccine RNA encoding vaccine antigen. Administering to the subject RNA encoding vaccine antigen may provide (following expression of the RNA by appropriate target cells) vaccine antigen for inducing an immune response against vaccine antigen (and disease-associated antigen) in the subject.

The present disclosure provides, among other things, a number of insights for achieving effective delivery of multiple antigenic polypeptides (e.g., antigenic polypeptides from different infectious agents) to a subject and/or provide robust immune responses against different infectious diseases. In some embodiments, the present disclosure provides insights relating to RNA vaccine technologies, including certain insights relating to antigen combinations, sequences used to encode antigenic polypeptides, non-coding elements, nanoparticle formulations, pharmaceutical compositions, and dosing regimens that can provide effective delivery of multiple antigenic polypeptides (e.g., antigenic polypeptides from different infectious agents) to a subject and/or provide robust immune responses against different infectious diseases.

In some embodiments, insights provided herein result in RNA compositions that can produce effective immune responses against multiple infectious agents. In some embodiments, insights provided herein result in RNA compositions that can produce immune responses against at least two infectious agents that are comparable to (e.g., within 70%, 80%, 90%, 95% or higher and up to 100%) or superior to (e.g., increased by at least 30%, including, e.g., at least 40%, at least 50%, at least 60%, at least 70%, at least 80%, at least 90%, at least 95%, at least 1.1-fold, at least 1.5-fold, at least 2-fold, at least 3-fold, or higher) the efficacy of their respective stand-alone (e.g., monovalent) RNA vaccines or reference vaccines such as non-RNA vaccines (e.g., inactivated virus vaccines). In some embodiments, insights provided herein result in RNA compositions that can produce superior (e.g., increased by at least 30%, including, e.g., at least 40%, at least 50%, at least 60%, at least 70%, at least 80%, at least 90%, at least 95%, at least 1.1-fold, at least 1.5-fold, at least 2-fold, at least 3-fold, or higher) immune responses against at least one and, in embodiments, each, of at least two infectious agents, as compared to the efficacy of their respective stand alone (e.g., monovalent) RNA vaccines even when the same dose of RNAs as in respective stand alone RNA vaccines are administered. In some embodiments, insights provided herein can even be used to produce RNA compositions comprising RNAs encoding at least two antigenic polypeptides of at least two infectious agents, each in a lower dose than as used in their respective stand alone (e.g., monovalent) RNA vaccines, which can produce superior (e.g., increased by at least 30%, including, e.g., at least 40%, at least 50%, at least 60%, at least 70%, at least 80%, at least 90%, at least 95%, at least 1.1-fold, at least 1.5-fold, at least 2-fold, at least 3-fold, or higher) immune responses against at least one and, in embodiments, each, of at least two infectious agents, as compared to that of their stand alone (e.g., monovalent) RNA vaccines having higher doses.

In some embodiments, the present disclosure provides an insight that using the same RNA backbone construct (e.g., having the same combination of non-coding elements, e.g., in the context of mRNA, the same 5′cap, cap proximal sequence, 5′ UTR sequence, 3′ UTR sequence, and polyA sequence, except for a sequence that encodes an antigenic payload) and/or using the same nanoparticle formulation that encapsulate RNAs (e.g., same lipid formulation), to deliver one or more antigenic polypeptides from at least two different infectious agents (e.g., a respiratory infectious agents) in a single composition can provide one or more certain advantages. For example, in some embodiments, such an approach may allow such RNAs to remain stable in the single composition after it is stored at non-zero temperatures or above for at least 24 hours or longer (e.g., in some embodiments, exposing to 30° C. for a period of time, followed by maintaining at 2-8° C. for a period of time). In some embodiments, the present disclosure provides an insight that using the same RNA backbone construct (e.g., having the same combination of non-coding elements, e.g., in the context of mRNA, the same 5′cap, cap proximal sequence, 5′ UTR sequence, 3′ UTR sequence, and polyA sequence, except for a sequence that encodes an antigenic payload) and/or using the same nanoparticle formulation that encapsulate RNAs (e.g., same lipid formulation), to deliver one or more antigenic polypeptides from at least two different infectious agents (e.g., a respiratory infectious agents) in a single composition can provide comparable pharmacokinetics and/or pharmacodynamics of such RNAs and/or can reduce or minimize interference between the immunogenicity of the encoded antigenic polypeptides, as compared to RNAs having a different combination of non-coding elements and/or different nanoparticle formulation.

In some embodiments, the present disclosure provides an insight that for compositions comprising two or more polynucleotides, each comprising a nucleotide sequence encoding an antigenic polypeptide associated with a different infectious agent, a superior immune response against each target infectious agent can be induced when the two or more polynucleotides comprise the same combination of non-coding elements (e.g., in the context of mRNA, the same 5′cap, cap proximal sequence, 5′ UTR sequence, 3′ UTR sequence, and polyA sequence, except for a sequence that encodes an antigenic payload) and/or formulated in the same nanoparticle formulation (e.g., same lipid formulation), as compared to RNAs having a different combination of non-coding elements and/or different nanoparticle formulation.

In some embodiments, the present disclosure provides an insight that multivalency of a combination vaccine may have an adjuvanting effect. Without wishing to be bound by a particular theory, in some embodiments, such an adjuvanting effect may be due to an increased concentration of nanoparticles (e.g., lipid nanoparticles) encapsulating RNAs that could lead to a dose-sparing effect. Accordingly, in some embodiments, in compositions comprising two or more polynucleotides, each comprising a nucleotide sequence encoding an antigen associated with a different infectious agent, and each formulated (together or separately) in a nanoparticle formulation (e.g., a lipid nanoparticle formulation), an immune response can be induced that is superior to that induced by a corresponding monovalent composition (e.g., a composition comprising only one of the polynucleotides in the same nanoparticle formulation). In some embodiments, the immune response induced by such compositions can be stronger than that induced by the same amount of a monovalent composition. In some embodiments, a lower amount of such compositions may be required to produce the same strength immune response as a monovalent product.

Without wishing to be bound by any particular theory, the present application provides an insight that, in some embodiments, separate encapsulation of individual mRNAs (e.g., in delivery vehicle(s), such as LNP(s)) that encode distinct antigenic polypeptides (e.g., antigenic polypeptides from different infectious agents—e.g., SARS-CoV-2 vs influenza, or antigenic polypeptides representing different variants of the same infectious agent—e.g., different variants of a SARS-CoV-2 spike protein, etc), may provide certain advantages including, for example, certain immunological benefits, for example as compared to co-encapsulation (e.g., two or more individual mRNAs that encode distinct antigenic polypeptides) in the same delivery vehicle(s). For example, the present disclosure proposes that, in some embodiments, separate encapsulation may achieve improved expression of one or more of encoded antigenic polypeptides, and/or improved immune responses against one or more encoded antigenic polypeptides. Without wishing to be bound by any particular theory, in some embodiments, separate encapsulation can facilitate separate uptake of individual mRNAs into cells in a subject (e.g., separate APCs). Without wishing to be bound by any particular theory, the present application notes that co-encapsulation of two or more RNAs, each encoding a different antigenic polypeptide, has the potential to lead to co-uptake by a cell (e.g., an APC), and that co-uptake could result in translational competition and reduced expression and/or a reduced immune response for one or more of the encoded antigenic polypeptides. In some embodiments, separate encapsulation of mRNAs encoding influenza antigens (e.g., separate encapsulation of each mRNA encoding an influenza antigen; encapsulation of mRNA(s) encoding an influenza type A antigen in a first population of nanoparticles (e.g., LNPs) and encapsulation of mRNA(s) encoding an influenza type B antigen in a second population of nanoparticles (e.g., LNPs); or encapsulation of mRNA(s) encoding an influenza type A antigen in a first population of nanoparticles (e.g., LNPs) and encapsulation of each mRNA encoding an influenza type B antigen in a separate population of nanoparticles (e.g., LNPs)) can provide benefits as compared to compositions comprising coencapsulated mRNAs encoding an influenza antigen. In some embodiments, a composition comprising (i) two or more different RNAS, each encoding an antigenic polypeptide (e.g., an HA protein) of an influenza type A virus (e.g., so that the two or more different RNAs, together, encode two or more different influenza A HA polypeptides), and (ii) two or more different RNAs, each encoding an antigenic polypeptide (e.g., an HA protein) of an influenza type B virus (e.g., so that the two or more different RNAs, together, encode two or more different influenza B HA polypeptides) are formulated in nanoparticles (e.g., LNPs) such that:
Each RNA encoding an antigenic polypeptide of an influenza type A virus is encapsulated in a first population of nanoparticles and each RNA encoding an antigenic polypeptide of an influenza type B virus is encapsulated in a second population of nanoparticles;
Each RNA is encapsulated in a separate nanoparticle; or
Each RNA encoding an influenza type A antigenic polypeptide is encapsulated in a first population of nanoparticles and the two RNAs encoding an influenza type B antigenic polypeptide are each encapsulated in separate populations of nanoparticles.

In some embodiments, the present disclosure also provides insights for producing an immune response that is broadly neutralizing against different influenza virus strains (e.g., that produces high neutralizing titers and/or seroconversion rates against influenza type A and/or type B viruses (e.g., neutralizing titers and/or seroconversion rates that are at clinically relevant levels (e.g., (i) neutralizing titers that are comparable or superior to those previously shown to prevent influenza symptoms, and/or (ii) neutralizing titers and/or seroconversion rates that are comparable or superior to those induced by a relevant comparator (e.g., a commercially approved influenza vaccine or an influenza RNA vaccine administered without a SARS-CoV-2 vaccine))). The present disclosure also provides exemplary doses of RNA that can produce strong immune responses against both types of influenza viruses (e.g., neutralizing titers and/or seroconversion rates that are at clinically relevant levels (e.g., (i) neutralizing titers that are comparable or superior to those previously shown to prevent influenza symptoms, and/or (ii) neutralizing titers and/or seroconversion rates that are comparable or superior to those induced by a relevant comparator (e.g., a commercially approved influenza vaccine or an influenza RNA vaccine administered without a SARS-CoV-2 vaccine))).

Coronaviruses are positive-sense, single-stranded RNA ((+)ssRNA) enveloped viruses that encode for a total of four structural proteins, spike protein(S), envelope protein (E), membrane protein (M) and nucleocapsid protein (N). The spike protein (S protein) is responsible for receptor-recognition, attachment to the cell, infection via the endosomal pathway, and the genomic release driven by fusion of viral and endosomal membranes. Though sequences between the different family members vary, there are conserved regions and motifs within the S protein making it possible to divide the S protein into two subdomains: S1 and S2. While the S2, with its transmembrane domain, is responsible for membrane fusion, the S1 domain recognizes the virus-specific receptor and binds to the target host cell. Within several coronavirus isolates, the receptor binding domain (RBD) was identified and a general structure of the S protein defined (FIG. 1).

In December 2019, a pneumonia outbreak of unknown cause occurred in Wuhan, China and it became clear that a novel coronavirus (severe acute respiratory syndrome coronavirus 2; SARS-CoV-2) was the underlying cause. The genetic sequence of SARS-CoV-2 became available to the WHO and public (MN908947.3) and the virus was categorized into the betacoronavirus subfamily. By sequence analysis, the phylogenetic tree revealed a closer relationship to severe acute respiratory syndrome (SARS) virus isolates than to another coronavirus infecting humans, namely the Middle East respiratory syndrome (MERS) virus.

SARS-CoV-2 infections and the resulting disease COVID-19 have spread globally, affecting a growing number of countries. On 11 Mar. 2020 the WHO characterized the COVID-19 outbreak as a pandemic. As of 1 Dec. 2020, there have been >63 million globally confirmed COVID-19 cases and >1.4 million deaths, with 191 countries/regions affected. The ongoing pandemic remains a significant challenge to public health and economic stability worldwide.

Every individual is at risk of infection as there is no pre-existing immunity to SARS-CoV-2. Following infection some but not all individuals develop protective immunity in terms of neutralising antibody responses and cell mediated immunity. However, it is currently unknown to what extent and for how long this protection lasts. According to WHO 80% of infected individuals recover without need for hospital care, while 15% develop more severe disease and 5% need intensive care. Increasing age and underlying medical conditions are considered risk factors for developing severe disease.

The presentation of COVID-19 is generally with cough and fever, with chest radiography showing ground-glass opacities or patchy shadowing. However, many patients present without fever or radiographic changes, and infections may be asymptomatic which is relevant to controlling transmission. For symptomatic subjects, progression of disease may lead to acute respiratory distress syndrome requiring ventilation and subsequent multi-organ failure and death. Common symptoms in hospitalized patients (in order of highest to lowest frequency) include fever, dry cough, shortness of breath, fatigue, myalgias, nausea/vomiting or diarrhoea, headache, weakness, and rhinorrhea. Anosmia (loss of smell) or ageusia (loss of taste) may be the sole presenting symptom in approximately 3% of individuals who have COVID-19.

All ages may present with the disease, but notably case fatality rates (CFR) are elevated in persons >60 years of age. Comorbidities are also associated with increased CFR, including cardiovascular disease, diabetes, hypertension, and chronic respiratory disease. Healthcare workers are overrepresented among COVID-19 patients due to occupational exposure to infected patients.

In most situations, a molecular test is used to detect SARS-CoV-2 and confirm infection. The reverse transcription polymerase chain reaction (RT-PCR) test methods targeting SARS-CoV-2 viral RNA are the gold standard in vitro methods for diagnosing suspected cases of COVID-19. Samples to be tested are collected from the nose and/or throat with a swab.

Influenza is a major cause of morbidity and mortality worldwide, occurring in annual seasonal epidemics and occasionally in global pandemics (Cunha B A. Influenza: historical aspects of epidemics and pandemics. Infect Dis Clin North Am. 2004; 18(1):141-55). Symptomatic influenza virus infection causes a febrile illness with respiratory and systemic symptoms (Monto A S, Gravenstein S, Elliott M, et al. Clinical signs and symptoms predicting influenza infection. Arch Intern Med. 2000; 160(21):3243-7), although influenza virus infection is also often asymptomatic (Cowling B J, Chan K H, Fang V J, et al. Comparative epidemiology of pandemic and seasonal influenza A in households. N Engl J Med. 2010; 362(23):2175-84). The risk of complications and hospitalization from influenza are higher in people ≥65 years of age, young children, and people with certain underlying medical conditions. In the US, an average of >200,000 hospitalizations per year are related to influenza, while the annual global number of deaths is estimated to range from almost 300,000 to over 600,000 (Iuliano A D, Roguski K M, Chang H H, et al. Estimates of global seasonal influenza-associated respiratory mortality: a modelling study. Lancet. 2018; 391(10127):1285-300).

### Signs and Symptoms

Seasonal influenza is characterized by a sudden onset of fever, cough (usually dry), headache, muscle and joint pain, severe malaise (feeling unwell), sore throat and a runny nose. The cough can be severe and can last 2 or more weeks. Most people recover from fever and other symptoms within a week without requiring medical attention, but influenza can cause severe illness or death especially in people at high risk.

In industrialized countries most deaths associated with influenza occur among people age 65 or older. Epidemics can result in high levels of worker/school absenteeism and productivity losses. Clinics and hospitals can be overwhelmed during peak illness periods.

The effects of seasonal influenza epidemics in developing countries are not fully known, but research estimates that 99% of deaths in children under 5 years of age with influenza related lower respiratory tract infections are found in developing countries.

### Epidemiology

All age groups can be affected by influenza infection, but some groups are more at risk than others. People at greater risk of severe disease or complications when infected include: pregnant women, children under 59 months, the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy).

Health care workers are also at high risk of acquiring an influenza virus infection, due to patient exposure, and can also further spread the disease particularly to vulnerable individuals.

Seasonal influenza is transmitted easily, with rapid transmission in crowded areas including schools and nursing homes. When an infected person coughs or sneezes, droplets containing viruses (infectious droplets) are dispersed into the air and can spread up to one meter, and infect persons in close proximity who breathe these droplets in. The virus can also be spread by hands contaminated with influenza viruses. To prevent transmission, people should cover their mouth and nose with a tissue when coughing, and wash their hands regularly.

In temperate climates, seasonal epidemics occur mainly during winter, while in tropical regions, influenza may occur throughout the year, causing outbreaks more irregularly. The time from infection to illness, known as the incubation period, is about 2 days, but ranges from one to four days.

### Diagnosis

Most cases of human influenza are clinically diagnosed. Collection of appropriate respiratory samples and the application of a laboratory diagnostic test is typically recommended in order to establish a definitive diagnosis. Proper collection, storage and transport of respiratory specimens is typically the first step for laboratory detection of influenza virus infections. Commonly, influenza infection is confirmed using samples from throat, nasal and nasopharyngeal secretions or tracheal aspirate or washings, e.g., using direct antigen detection, virus isolation, or detection of influenza-specific RNA by reverse transcriptase-polymerase chain reaction (RT-PCR). Guidance on laboratory techniques is known in the art, and can be found, for example on the World Health Organization's (WHO) website. Rapid influenza diagnostic tests (RIDTs) can be used in clinical settings, but can have a lower sensitivity as compared to RT-PCR methods and their reliability depends largely on the conditions under which they are used. Among other things, the present disclosure provides insights into immune responses elicited by compositions comprising (i) two or more antigenic polypeptides, each associated with different infectious agents, or (ii) two or more polynucleotides, each comprising a sequence encoding an antigenic polypeptide associated with a different infectious agent. These insights enable the development of combination products and/or treatments that can induce a strong immune response against multiple infectious agents (e.g., combination products that induce an immune response that is similar to, or even superior to that induced by monovalent products). In particular, the present disclosure provides insights into immune responses elicited by compositions comprising (i) one or more antigenic polypeptides associated with a coronavirus and one or more antigenic polypeptides associated with an influenza virus, or (ii) one or more polynucleotides, each comprising a nucleotide sequence encoding an antigenic polypeptide associated with an influenza virus.

In some embodiments, the present disclosure provides a composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 9;
(ii) an RNA comprising a second nucleotide sequence that includes modified uridines and encodes a second SARS-CoV-2 Spike (S) polypeptide from a variant of the SARS-CoV-2 strain, wherein the second nucleotide sequence is at least 85% identical to SEQ ID NO: 70;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 92;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 97;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 102; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 107.

In some embodiments, the present disclosure provides a composition comprising:
(i) an RNA comprising a nucleotide sequence that includes modified uridines and encodes a first SARS-CoV-2 Spike (S) polypeptide from a SARS-CoV-2 strain, wherein the RNA comprises a nucleotide sequence that is at least 85% identical to SEQ ID NO: 20;
(ii) an RNA comprising a nucleotide sequence that includes modified uridines and encodes a second SARS-CoV-2 Spike (S) polypeptide from a variant of the SARS-CoV-2 strain, wherein the RNA comprises a nucleotide sequence that is at least 85% identical to SEQ ID NO: 72;
(iii) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94;
(iv) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99;
(v) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104; and
(vi) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109.

In some embodiments, the influenza A H1N1 strain is Influenza A/Wisconsin/588/2019. In some embodiments, the influenza A H3N2 strain is Influenza A/Cambodia/e0826360/2020. In some embodiments, the influenza B Victoria strain is Influenza B/Washington/02/2019. In some embodiments, the influenza B Yamagata strain is Influenza B/PHUKET/3073/2013.

In some embodiments, disclosed herein is a composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 9;
(ii) an RNA comprising a second nucleotide sequence that includes modified uridines and encodes a second SARS-CoV-2 Spike (S) polypeptide from a variant of the SARS-CoV-2 strain, wherein the second nucleotide sequence is at least 85% identical to SEQ ID NO: 70;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 92;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 82;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 87; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 107.

In some embodiments, the present disclosure provides a composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 20;
(ii) an RNA comprising a second nucleotide sequence that includes modified uridines and encodes a second SARS-CoV-2 Spike (S) polypeptide from a SARS-CoV-2 strain, wherein the first nucleotide sequence is at least 85% identical to SEQ ID NO: 72;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 94;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 84;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 89; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 109.

In some embodiments, an influenza A H1N1 strain is Influenza A/Wisconsin/588/2019. In some embodiments, an influenza A H3N2 strain is Influenza A/Darwin/6/2021. In some embodiments, an influenza B Victoria strain is Influenza B/Austria/1359417/2021. In some embodiments, an influenza B Yamagata strain is Influenza B/PHUKET/3073/2013.

In some embodiments, a SARS-CoV-2 strain is a Wuhan strain. In some embodiments, a variant of the SARS-CoV-2 strain is an Omicron BA.4/5 variant. In some embodiments, a variant of the SARS-CoV-2 strain is an Omicron XBB.1.5 variant.

In some embodiments, each RNA in a composition comprises the same non-coding elements, including the same 5′ cap, cap proximal sequence, 5′ UTR sequence, 3′ UTR sequence, and polyA sequence.

In some embodiments, the mass ratio of RNAS (i)-(ii) to RNAs (iii)-(vi) is 1:5 to 5:1, 1:4 to 4:1, 1:3 to 3:1, or 1:2 to 2:1. In some embodiments, the mass ratio RNAs (iii)-(iv) to RNAs (v)-(vi) is 1:1 to 1:5. In some embodiments, the mass ratio of RNA (i) to RNA (ii) is 1:1. In some embodiments, RNAs (iii), (iv), (v), and (vi) are present at a mass ratio of 1:1:1:1 or 1:1:5:5.

In some embodiments, the combined mass of RNAs (i)-(vi) in a composition is about 30 μg to about 100 μg. In some embodiments, the combined mass of RNA (i) and RNA (ii) is about 3 μg to about 60 μg (e.g., about 3 μg, about 10 μg, about 30 μg, or about 60 μg). In some embodiments, the combined mass of RNAs (iii)-(vi) is about 30 μg to about 60 μg (e.g., about 30 μg or about 60 μg).

In some embodiments, RNA (i) and RNA (ii) are each present in an amount of about 15 μg, and RNAs (iii)-(vi) are each present in an amount of about 7.5 μg. In some embodiments, RNA (i) and (ii) are each present in an amount of about 30 μg, and RNAs (iii)-(vi) are each present in an amount of about 7.5 μg. In some embodiments, RNA (i) and (ii) are each present in an amount of about 15 μg, and RNAs (iii)-(vi) are each present in an amount of about 11.25 μg. In some embodiments, RNA (i) and (ii) are each present in an amount of about 15 μg, RNAs (iii) and (iv) are each present in an amount of about 5 μg, and RNAs (v) and (vi) are each present in an amount of about 25 μg. In some embodiments, RNA (i) and (ii) are each present in an amount of about 15 μg, RNAs (iii) and (iv) are each present in an amount of about 2.5 μg, and RNAs (v) and (vi) are each present in an amount of about 12.5 μg. In some embodiments, RNA (i) and (ii) are each present in an amount of about 30 μg, RNAs (iii) and (iv) are each present in an amount of about 2.5 μg, and RNAs (v) and (vi) are each present in an amount of about 12.5 μg. In some embodiments, RNA (i)-(vi) are each present in an amount of about 15 μg.

In some embodiments, a composition comprises:
(i) an RNA comprising a first nucleotide sequence that includes modified uridines and encodes a SARS-CoV-2 Spike (S) polypeptide, wherein the first nucleotide sequence is at least 85% identical to SEQ ID NO: 129;
(ii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 92;
(iii) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 99;
(iv) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 102; and
(v) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 107.

In some embodiments, a composition comprises:
(i) an RNA comprising a nucleotide sequence that includes modified uridines and encodes a SARS-CoV-2 Spike (S) polypeptide, wherein the RNA comprises a nucleotide sequence that is at least 85% identical to SEQ ID NO: 132;
(ii) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94;
(iii) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99;
(iv) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104; and
(v) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109.

In some embodiments, a composition comprises an RNA encoding an hemagglutinin antigen from an influenza A H1N1 strain, wherein the influenza A H1N1 strain is Influenza A/Wisconsin/588/2019.

In some embodiments, a composition comprises an RNA encoding an hemagglutinin antigen from an influenza A H3N2 strain, wherein the influenza A H3N2 strain is Influenza A/Cambodia/e0826360/2020.

In some embodiments, a composition comprises an RNA encoding an hemagglutinin antigen from an influenza B Victoria strain, wherein the influenza B Victoria strain is Influenza B/Washington/02/2019.

In some embodiments, a composition comprises an RNA encoding an hemagglutinin antigen from an influenza B Yamagata strain, wherein the influenza B Yamagata strain is Influenza B/PHUKET/3073/2013.

In some embodiments, a composition comprises:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 130;
(ii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 92;
(iii) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 82;
(iv) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 87; and
(v) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 107.

In some embodiments, a composition comprises:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 132;
(iii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 94;
(iv) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 84;
(v) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 89; and
(vi) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 109.

In some embodiments, a composition comprises an RNA encoding an hemagglutinin antigen from an influenza A H3N2 strain, wherein the influenza A H3N2 strain is Influenza A/Darwin/6/2021.

In some embodiments, a composition comprises an RNA encoding an hemagglutinin antigen from an influenza B Victoria strain, wherein the influenza B Victoria strain is Influenza B/Austria/1359417/2021.

In some embodiments, a composition comprises an RNA encoding an hemagglutinin antigen from an influenza B Yamagata strain, wherein the influenza B Yamagata strain is Influenza B/PHUKET/3073/2013.

In some embodiments, a composition comprises an RNA encoding a SARS-CoV-2 S protein from a Wuhan strain.

In some embodiments, a composition comprises an RNA encoding a SARS-CoV-2 S protein from an Omicron BA.4/5 variant.

In some embodiments, a composition comprises an RNA encoding a SARS-CoV-2 S protein from an XBB.1.5 variant.

In some embodiments, each of the RNAs in the composition comprises the same non-coding elements (e.g., including the same 5′ cap, cap proximal sequence, 5′ UTR sequence, 3′ UTR sequence, and polyA sequence).

In some embodiments, a composition comprises RNA (i) and RNAs (ii)-(v) in a mass ratio of 1:5 to 5:1, 1:4 to 4:1, 1:3 to 3:1, or 1:2 to 2:1.

In some embodiments, a composition comprises RNA (i) and RNAs (ii)-(v) in a mass ratio of 1:1 to 1:5.

In some embodiments, a composition comprises RNAs (ii), (iii), (iv), and (v) in a mass ratio of 1:1:1:1 or 1:1:5:5.

In some embodiments, a composition comprises one or more RNAs encoding a SARS-CoV-2 S protein, and one or more RNAs encoding an influenza HA protein, wherein the mass ratio of (i) the one or more RNAs encoding a SARS-CoV-2 S protein to (ii) the one or more RNAs encoding an influenza HA protein is 1:5 to 5:1, 1:4 to 4:1, 1:3 to 3:1, or 1:2 to 2:1 (e.g., 1:2 or 2:1).

In some embodiments, a composition comprises one or more RNAs encoding a SARS-CoV-2 S protein, wherein the mass of the one or more RNAs is about 30 μg. In some embodiments, a composition comprises one or more RNAs encoding a SARS-CoV-2 S protein, wherein the mass of the one or more RNAs is about 60 μg.

In some embodiments, a composition comprises one or more RNAs encoding an influenza HA protein, wherein the mass of the one or more RNAs encoding an influenza HA protein is 30 μg. In some embodiments, a composition comprises one or more RNAs encoding an influenza HA protein, wherein the mass of the one or more RNAs encoding an influenza HA protein is 60 μg.

In some embodiments, a composition comprises:
(a) RNA (i) in an amount of about 30 μg, and RNAs (ii)-(v) in an amount of about 7.5 μg each;
(b) RNA (i) in an amount of about 60 μg, and RNAs (ii)-(v) in an amount of about 7.5 μg each;
(c) RNA (i) in an amount of about 30 μg, and RNAs (ii)-(v) in an amount of about 11.25 μg each;
(d) RNA (i) in an amount of about 30 μg, RNAs (ii) and (iii) in an amount of about 5 μg each, and RNAs (iv) and (v) in an amount of about 25 μg each;
(e) RNA (i) in an amount of about 30 μg, RNAs (ii) and (iii) in an amount of about 2.5 μg each, and RNAs (iv) and (v) in an amount of about 12.5 μg each;
(f) RNA (i) in an amount of about 30 μg, RNAs (ii) and (iii) in an amount of about 2.5 μg each, and RNAs (iv) and (v) in an amount of about 12.5 μg each; or
(g) RNA (i) in an amount of about 30 μg, and RNAs (ii)-(v) in an amount of about 15 μg each.

In some embodiments, a composition described herein comprises:
(i) a coronavirus RNA vaccine comprising one or more RNAs, each comprising a nucleotide sequence that encodes a SARS-CoV-2 antigen; and
(ii) an influenza RNA vaccine comprising one or more RNAs, each comprising one or more nucleotide sequences that encode an influenza antigen, wherein the influenza RNA vaccine encodes at least four influenza antigens, and wherein each influenza antigen is from a distinct influenza virus strain that is predicted to circulate during a flu season of a particular hemisphere;
wherein each RNA in the composition comprises the same non-coding elements, including the same 5′ cap, cap proximal sequence, 5′ UTR sequence, 3′ UTR sequence, and polyA sequence.

In some embodiments, each of the one or more RNAs in a coronavirus RNA vaccine and each of the one or more RNAs in an influenza RNA vaccine include one or more modified uridines.

In some embodiments, the present disclosure provides a composition comprising:
a coronavirus RNA vaccine that is at least bivalent, wherein the coronavirus RNA vaccine comprises one or more RNAs comprising a nucleotide sequence that encodes at least two SARS-CoV-2 antigens; and
an influenza RNA vaccine that is at least quadrivalent, wherein the influenza RNA vaccine comprises one or more RNAs comprising a nucleotide sequence that encodes at least four influenza antigens, each influenza antigen from a distinct influenza virus strain predicted to circulate during a flu season of a particular hemisphere;
wherein each RNA in the coronavirus RNA vaccine and in the influenza RNA vaccine comprises the same non-coding elements that include the same 5′ cap, cap proximal sequence, 5′ UTR sequence, 3′ UTR sequence, and polyA sequence.

In some embodiments, each RNA in an at least bivalent coronavirus RNA vaccine and each RNA in an at least quadrivalent influenza RNA vaccine includes modified uridines in place of uridine.

In some embodiments, an at least bivalent SARS-CoV-2 vaccine comprises or encodes at least two SARS-CoV-2 antigens that are or comprise a SARS-CoV-2 Spike (S) polypeptide from a SARS-CoV-2 strain and a SARS-CoV-2 S polypeptide from a variant of the SARS-CoV-2 strain.

In some embodiments, a quadrivalent influenza vaccine comprises or encodes at least four influenza antigens, each of which are or comprise a hemagglutinin antigen from a distinct influenza virus strain predicted to circulate during a flu season. In some embodiments, each distinct influenza virus is predicted to circulate during a flu season based on human serology data from the Northern or Southern hemisphere.

In some embodiments, a coronavirus RNA vaccine encodes at least two SARS-CoV-2 antigens, each from a distinct SARS-CoV-2 strain or variant.

In some embodiments, an at least bivalent SARS-CoV-2 vaccine comprises RNAs encoding at least two SARS-CoV-2 antigens, each of which are each encoded by a separate RNA.

In some embodiments, an at least quadrivalent influenza vaccine comprises RNAs encoding at least four influenza antigens, each of which is encoded by a separate RNA.

In some embodiments, RNAs in an at least bivalent coronavirus vaccine and RNAs in an at least tetravalent influenza vaccine are present in a mass ratio of 1:5 to 5:1, 1:4 to 4:1, 1:3 to 3:1, or 1:2 to 2:1.

In some embodiments, an at least quadrivalent influenza vaccine comprises RNA encoding at least four influenza antigens, including at least two hemagglutinin antigens from influenza A viruses and at least two hemagglutinin antigens from influenza B viruses, wherein each influenza antigen is encoded by a separate RNA. In some embodiments, RNAs that encode hemagglutinin antigens from influenza A viruses and RNAs that encode hemagglutinin antigens from influenza B viruses are present in a mass ratio of 1:1 to 1:5. In some embodiments, an at least quadrivalent influenza vaccine comprises at least four RNAs present in a mass ratio of 1:1:1:1.

In some embodiments, an at least bivalent coronavirus vaccine comprise at least two RNAs, each encoding a different coronavirus antigen, wherein the two RNAs are present in a mass ratio of 1:1.

In some embodiments, a composition comprises RNA in a total amount of about 30 μg to about 100 μg (e.g., about 30 μg, about 45 μg, about 60 μg, about 75 μg, or about 90 μg).

In some embodiments, a composition comprising an at least bivalent coronavirus vaccine and an at least quadrivalent influenza vaccine comprises a total amount of RNA of 30 μg to 100 μg.

In some embodiments, the present disclosure provides a composition comprising:
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent, wherein the second infectious agent is different from the first infectious agent;
wherein each of the first and second RNAs in the composition comprises the same non-coding elements, including the same 5′ cap, cap proximal sequence, 5′ UTR sequence, 3′ UTR sequence, and polyA sequence, and
wherein at least one of the same non-coding elements is or comprises:
   (i) a 5′-UTR sequence that is or comprises a modified human alpha-globin 5′-UTR;
   (ii) a 3′-UTR sequence that is or comprises a first sequence from the amino terminal enhancer of split (AES) messenger RNA and a second sequence from the mitochondrial encoded 12S ribosomal RNA;
   (iii) a polyA sequence comprising at least 100 A nucleotides, wherein the first RNA and the second RNA each do not comprise a stretch of at least 30 contiguous C nucleotides between the 3′ UTR and the polyA sequence;
   (iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
   (v) a 5′ cap comprising a Cap1 structure and a cap proximal sequence comprising positions +1, +2, +3, +4, and +5 of the RNA; wherein:
      (a) the Cap1 structure comprises m7(3′OMeG)(5′)ppp(5′)(2′OMeA₁)pG₂, wherein A₁ is position +1 of the RNA, and G₂ is position +2 of the RNA; and
      (b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

In some embodiments, the present disclosure provides a composition comprising:
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent that is different from the first infectious agent;
wherein each of the first and second RNAs in the composition comprises the same non-coding elements including the same 5′ cap, cap proximal sequence, 5′ UTR sequence, 3′ UTR sequence, and polyA sequence, and
wherein each of the first and second RNAs is characterized in that:
   (i) an immune response induced by the RNA in the composition has a level that is at least 80% of a level of an immune response induced by the same RNA when it is administered alone; and/or
   (ii) an immune response induced by the RNA in the composition has a level that is at least 80% of a level of an immune response induced by the same RNA when it is administered separately from the other RNAs at a different location of a subject's body; and/or
   (iii) an immune response induced by the RNA in the composition has a level that is at least 80% of a level of an immune response induced by a respective reference composition.

In some embodiments, a respective reference composition is an inactivated virus vaccine.

In some embodiments, an immune response induced by one or more first RNA(s) and one or more second RNA(s) are each at least 100% of a level of an immune response induced by the same RNA when the one or more first RNA(s) and the one or more second RNA(s) are administered separately.

In some embodiments, an immune response induced by one or more first RNA(s) and one or more second RNA(s) are each greater than an immune response induced by the same RNAs administered separately.

In some embodiments, one or more first RNA(s) and one or more second RNA(s) are each present at a dose that is lower than that of the same RNAs administered separately, wherein the immune response induced by the lower dose of the one or more first RNA(s) and the one or more second RNA(s) are each substantially comparable to or greater than the immune response induced by a greater dose of the same RNAs administered separately.

In some embodiments, disclosed herein is a composition comprising:
one or more first RNAs each comprising a nucleotide sequence that encodes one or more antigenic polypeptides associated with a first infectious agent;
one or more second RNAs each comprising a nucleotide sequence that encodes one or more antigenic polypeptides associated with a second infectious agent that is different from the first infectious agent;
wherein each of the first and second RNAs in the composition comprises the same non-coding elements, including the same 5′ cap, cap proximal sequence, 5′ UTR sequence, 3′ UTR sequence, and polyA sequence,
wherein each of the first and second RNAs is encapsulated, separately or together, in nanoparticles; and
wherein the composition is characterized in that:
   (i) RNA content of the composition is at least 95% that of the initial RNA content after storing for 24 hours;
   (ii) RNA encapsulation remains at least 95% that of the initial RNA encapsulation after storing for 24 hours;
   (iii) the nanoparticles encapsulating the first and second RNAs have maintained substantially the same size after storing for 24 hours;
   (iv) the nanoparticles encapsulating the first and second RNAs have maintained a polydispersity of no more than 0.3 after 24 hours; and/or
   (v) the mass ratio of the first RNA and the second RNA remains substantially the same after storing for 24 hours.

In some embodiments, compositions disclosed herein comprise nanoparticles that comprise lipid nanoparticles, polyplexes (PLX), lipidated polyplexes (LPLX), liposomes, or polysaccharide nanoparticles. In some embodiments, compositions disclosed herein comprise nanoparticles that comprise lipid nanoparticles. In some embodiments, lipid nanoparticles each comprise: a cationically ionizable lipid; and one or more neutral lipids, and a polymer-conjugated lipid. In some embodiments, a polymer-conjugated lipid comprises a PEG-conjugated lipid. In some embodiments, nanoparticles have an average diameter of about 50-150 nm.

In some embodiments, for each of (i)-(v), the first 12 hours of storing is at 30° C. and the remaining 12 hours of storing is at 2-8° C.

In some embodiments, one or more first RNAs comprise at least two first RNAs, each comprising a nucleotide sequence that encodes one or more antigenic polypeptides associated with a different strain or variant of a first infectious agent.

In some embodiments, one or more second RNAs comprise at least two second RNAs, each comprising a nucleotide sequence that encodes one or more antigenic polypeptides associated with a different strain or variant of a second infectious agent.

In some embodiments, one or more second RNAs comprise at least three second RNAs, each comprising a nucleotide sequence that encodes one or more antigenic polypeptides associated with a different strain or variant of a second infectious agent.

In some embodiments, one or more second RNAs comprise at least four second RNAs, each comprising a nucleotide sequence that encodes one or more antigenic polypeptides associated with a different variant or strain of a second infectious agent.

In some embodiments, disclosed herein is a composition comprising:
a plurality of (e.g., at least two, at least three, at least four, or at least five or more) first RNAs each comprising a nucleotide sequence that encodes one or more antigenic polypeptides associated with a first infectious agent of a different strain and/or variant thereof;
one or more second RNAs each comprising a nucleotide sequence that encodes one or more antigenic polypeptides associated with a second infectious agent that is different from the first infectious agent;
and wherein each of the first and second RNAs is formulated, either separately or together, in the same nanoparticle formulation;
wherein (i) the first RNAs and the second RNAs are present in a mass ratio of 1:2 to 2:1 and/or (ii) the first RNAs and second RNAs are present in the total amount of about 10 μg to about 100 μg per dose; and
   one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent, wherein the second infectious agent is different from the first infectious agent;
wherein each of the first and second RNAs in the composition comprises the same non-coding elements, including the same 5′ cap, cap proximal sequence, 5′ UTR sequence, 3′ UTR sequence, and polyA sequence, and
wherein at least one of the same non-coding elements is or comprises:
   (i) a 5′-UTR sequence that is or comprises a modified human alpha-globin 5′-UTR;
   (ii) a 3′-UTR sequence that is or comprises a first sequence from the amino terminal enhancer of split (AES) messenger RNA and a second sequence from the mitochondrial encoded 12S ribosomal RNA;
   (iii) a polyA sequence comprising at least 100 A nucleotides, wherein the first RNA and the second RNA each do not comprise a stretch of at least 30 contiguous C nucleotides between the 3′ UTR and the polyA sequence;
   (iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
   (v) a 5′ cap comprising a Cap1 structure and a cap proximal sequence comprising positions +1, +2, +3, +4, and +5 of the RNA; wherein:
      (a) the Cap1 structure comprises m7(3′OMeG)(5′)ppp(5′)(2′OMeA₁)pG₂, wherein A₁ is position +1 of the RNA, and G₂ is position +2 of the RNA; and
      (b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

In some embodiments, each first RNA in a composition is co-formulated in the same nanoparticle formulation. In some embodiments, each second RNA in a composition is co-formulated in the same nanoparticle formulation. In some embodiments, each first RNA and each second RNA in a composition are formulated in separate populations of nanoparticles. In some embodiments, each first RNA and each second RNA in a composition are co-formulated together in the same nanoparticle formulation.

In some embodiments, a first infectious agent is or comprises a coronavirus.

In some embodiments, a composition comprises one or more first RNAs comprising (i) an RNA comprising a nucleotide sequence that encodes one or more antigenic polypeptides associated with a first coronavirus and (ii) an RNA comprising a nucleotide sequence that encodes one or more antigenic polypeptides associated with a second coronavirus.

In some embodiments, a composition comprises a plurality of second RNAs, each comprising a nucleotide sequence that encodes one or more antigenic polypeptides associated with a second infectious agent of a different strain and/or variant thereof.

In some embodiments, a composition comprises at least two second RNAs, each comprising a nucleotide sequence that encodes one or more antigenic polypeptides associated with a second infectious agent of a different strain and/or variant thereof.

In some embodiments, a composition comprises at least three second RNAs, each comprising a nucleotide sequence that encodes one or more antigenic polypeptides associated with a second infectious agent of a different strain and/or variant thereof.

In some embodiments, a composition comprises at least four second RNAs, each comprising a nucleotide sequence that encodes one or more antigenic polypeptides associated with a second infectious agent of a different strain and/or variant thereof.

In some embodiments, a second infectious agent is or comprises a bacterial infectious agent. In some embodiments, a bacterial infectious agent is Streptococcus pneumoniae.

In some embodiments, a second infectious agent is or comprises a viral infectious agent. In some embodiments, a viral infectious agent induces an infectious respiratory disease. In some embodiments, a viral infectious agent is or comprises an influenza virus, a pneumoviridae virus, or a Paramyxoviridae virus. In some embodiments, a Pneumoviridae virus is a Respiratory syncytial virus (RSV). In some embodiments, an infectious respiratory disease is or comprises an influenza type A, type B, and/or type C virus. In some embodiments, an infectious respiratory disease is or comprises an influenza type A, and/or type B virus.

In some embodiments, a composition comprises (i) at least one RNA comprising a nucleotide sequence that encodes one or more antigenic polypeptides associated with influenza type A virus and (ii) at least one RNA comprising a nucleotide sequence that encodes one or more antigenic polypeptides associated with influenza type B virus.

In some embodiments, a composition comprises at least two RNAs, each comprising a nucleotide sequence that encodes one or more antigenic polypeptides associated with a different strain of an influenza type A virus, and at least two RNAs, each comprising a nucleotide sequence that encodes one or more antigenic polypeptides associated with a different strain of an influenza type B virus.

In some embodiments, an antigenic polypeptide(s) associated with an influenza virus an Hemagglutinin (HA) polypeptide, a neuraminidase (NA) polypeptide, or combinations thereof, or immunogenic fragments thereof.

In some embodiments, strain(s) of an influenza type A and/or influenza type B viruses have been predicted to be or are circulating strains in a coming flu season, for example, based on human serology data.

In some embodiments, strain(s) of an influenza A virus are selected from an H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, and an H10N8 virus. In some embodiments, strain(s) of an influenza type A virus is selected from an H1N1, H3N2, H5N1, and an H5N8 virus.

In some embodiments, a composition comprises one or more second RNAs comprising an RNA comprising a nucleotide sequence encoding an antigenic polypeptide associated with an H1N1 virus. In some embodiments, an H1N1 virus is A/Wisconsin/588/2019. In some embodiments, an antigenic polypeptide associated with A/Wisconsin/588/2019 is an HA polypeptide and comprises an amino acid sequence that is at least 85% identical to SEQ ID NO: 90. In some embodiments, an antigenic polypeptide associated with A/Wisconsin/588/2019 is an HA polypeptide and an RNA encoding the HA polypeptide comprises a nucleotide sequence that is at least 85% identical to SEQ ID NO: 92.

In some embodiments, a composition comprises one or more second RNAs comprising an RNA comprising a nucleotide sequence encoding an antigenic polypeptide associated with an H3N2 virus. In some embodiments, an H3N2 virus is A/Cambodia/e0826360/2020. In some embodiments, an antigenic polypeptide associated with A/Cambodia/e0826360/2020 is an HA polypeptide and comprises an amino acid sequence that is at least 85% identical to SEQ ID NO: 95. In some embodiments, an antigenic polypeptide associated with A/Cambodia/e0826360/2020 is an HA polypeptide, and an RNA encoding the HA polypeptide comprises a nucleotide sequence that is at least 85% identical to SEQ ID NO: 92. In some embodiments, an H3N2 virus is A/Darwin/6/2021. In some embodiments, an antigenic polypeptide associated with A/Darwin/6/2021 is an HA polypeptide and comprises an amino acid sequence that is at least 85% identical to SEQ ID NO: 80. In some embodiments, an antigenic polypeptide associated with A/Darwin/6/2021 is an HA polypeptide and an RNA encoding the HA polypeptide comprises a nucleotide sequence that is at least 85% identical to SEQ ID NO: 82.

In some embodiments, a composition comprises one or more second RNAs comprising an RNA comprising a nucleotide sequence encoding an antigenic polypeptide associated with a B/Yamagata or B/Victoria lineage virus.

In some embodiments, a B/Victoria lineage influenza virus is B/Washington/02/2019. In some embodiments, an antigenic polypeptide associated with B/Washington/02/2019 is an HA polypeptide and comprises an amino acid sequence that is at least 85% identical to SEQ ID NO: 100. In some embodiments, an antigenic polypeptide associated with B/Washington/02/2019 is an HA polypeptide, and an RNA encoding the HA polypeptide comprises a nucleotide sequence that is at least 85% identical to SEQ ID NO: 102. In some embodiments a B/Victoria lineage influenza virus is B/Austria/1359417/2021. In some embodiments, an antigenic polypeptide associated with B/Austria/1359417/2021 is an HA polypeptide and comprises a sequence that is at least 85% identical to SEQ ID NO: 85. In some embodiments, the antigenic polypeptide associated with B/Austria/1359417/2021 is an HA polypeptide and an RNA encoding the HA polypeptide comprises a nucleotide sequence that is at least 85% identical to SEQ ID NO: 87.

In some embodiments, a B/Yamagata lineage influenza virus is B/Phuket/3073/2013. In some embodiments, an antigenic polypeptide associated with B/Phuket/3073/2013 is an HA polypeptide and comprises an amino acid sequence that is at least 85% identical to SEQ ID NO: 105. In some embodiments, an antigenic polypeptide associated with B/Phuket/3073/2013 is an HA polypeptide, and an RNA encoding an HA polypeptide comprises a nucleotide sequence that is at least 85% identical to SEQ ID NO: 107.

In some embodiments, a first infectious agent is a coronavirus. In some embodiments, a coronavirus is an alphacoronavirus, a betacoronavirus, a gammacoronavirus, or a deltacoronavirus. In some embodiments, a coronavirus is a betacoronavirus. In some embodiments, a betacoronavirus is a sarbecovirus, a merbecovirus, an embecorvius, a nobecovirus, or a hibecorvirus. In some embodiments, a sarbecovirus is SARS-CoV-1 or SARS-CoV-2. In some embodiments, a sarbecovirus is SARS-CoV-2. In some embodiments, a merbecovirus is MERS-CoV.

In some embodiments, a composition comprises one or more first RNAs comprising an RNA comprising a nucleotide sequence that encodes one or more antigenic polypeptides associated with a SARS-CoV-2 variant that is prevalent or has been identified as a variant of concern in a relevant population at the time of administration.

In some embodiments, a composition comprises one or more first RNAs comprising an RNA comprising a nucleotide sequence that encodes one or more antigenic polypeptides associated with an Omicron SARS-CoV-2 variant (e.g., a BA.1, BA.2, BA.4/5, or XBB.1.5 variant).

In some embodiments, a composition disclosed herein comprises (i) an RNA comprising a nucleotide sequence that encodes one or more antigenic polypeptides associated with a first SARS-CoV-2 strain, wherein the first SARS-CoV-2 strain is a SARS-CoV-2 ancestral strain (Wuhan strain) and (ii) an RNA comprising a nucleotide sequence that encodes one or more antigenic polypeptides associated with a second SARS-CoV-2 variant, wherein the second SARS-CoV-2 is a variant of the SARS-CoV-2 ancestral strain, and is prevalent or has been identified as a variant of concern in a relevant population at the time of administration.

In some embodiments, a composition comprises (i) an RNA comprising a nucleotide sequence that encodes one or more antigenic polypeptides associated with a first SARS-CoV-2 variant and (ii) an RNA comprising a nucleotide sequence that encodes one or more antigenic polypeptides associated with a second SARS-CoV-2 variant, wherein the first and the second SARS-CoV-2 variant are each prevalent or have been identified as a variant of concern in a relevant population at the time of administration. In some embodiments, a second SARS-CoV-2 variant is an Omicron variant of SARS-CoV-2. In some embodiments, an Omicron variant of SARS-CoV-2 is or comprises Omicron BA.1, BA.2, or BA.4/5. In some embodiments, the antigenic polypeptide(s) associated with the coronavirus is a Spike (S) polypeptide, or a immunogenic fragment or variant thereof. In some embodiments, an S polypeptide is a prefusion stabilized S polypeptide. In some embodiments, a prefusion stabilized S polypeptide comprises at least two proline substitutions. In some embodiments, the two proline substitutions comprises proline residues at positions corresponding to residues 986 and 987 of SEQ ID NO: 1. In some embodiments, a prefusion stabilized S polypeptide comprises at least six proline substitutions. In some embodiments, a prefusion stabilized S polypeptide comprises proline residues at positions corresponding to residues 817, 892, 899, and 942 of SEQ ID NO: 1. In some embodiments, an RNA encoding one or more antigenic polypeptides associated with an Omicron SARS-CoV-2 variant encodes an S protein associated with an XBB.1.5 strain and comprises an amino acid sequence that is at least 85% identical to SEQ ID NO: 129.

In some embodiments, RNA encoding one or more antigenic polypeptides associated with a SARS-CoV-2 ancestral strain encodes an S protein associated with a Wuhan strain and comprising an amino acid sequence that is at least 85% identical to SEQ ID NO: 1. In some embodiments, RNA encoding SEQ ID NO: 1 comprises a nucleotide sequence that is at least 85% identical to SEQ ID NO: 9.

In some embodiments, RNA encoding one or more antigenic polypeptides associated with a second SARS-CoV-2 variant encode an S protein associated with a BA.4/5 variant, and comprise an amino acid sequence that is at least 85% identical to SEQ ID NO: 69. In some embodiments, an RNA encoding SEQ ID NO: 69 comprises a nucleotide sequence that is at least 85% identical to SEQ ID NO: 70.

In some embodiments, a composition disclosed herein comprises:
(i) (a) an RNA comprising a nucleotide sequence that encodes a SARS-CoV-2 Spike (S) polypeptide from an Omicron variant of SARS-CoV-2 (e.g., a BA.1, BA.2, BA.4/5, or XBB.1.5 variant) or (b) an RNA comprising a nucleotide sequence that encodes a SARS-CoV-2 Spike (S) polypeptide from a SARS-CoV-2 ancestral strain (Wuhan strain) and an RNA comprising a nucleotide sequence that encodes a SARS-CoV-2 Spike (S) polypeptide from an Omicron variant of SARS-CoV-2 (e.g., a BA.1, BA.2, BA.4/5, or XBB.1.5 variant); and
(ii) an RNA comprising a nucleotide sequence that encodes an HA polypeptide from an influenza A/H1N1 virus, an RNA comprising a nucleotide sequence that encodes an HA polypeptide from an influenza A/H3N2 virus, an RNA comprising a nucleotide sequence that encodes an HA polypeptide from an influenza B/Victoria lineage virus, and an RNA comprising a nucleotide sequence that encodes an HA polypeptide from an influenza B/Yamagata virus.

In some embodiments, an H1N1 virus is A/Wisconsin/588/2019. In some embodiments, an HA polypeptide associated with A/Wisconsin/588/2019 comprises a sequence that is at least 85% identical to SEQ ID NO: 90. In some embodiments, an HA polypeptide associated with A/Wisconsin/588/2019 is encoded by an RNA comprising a nucleotide sequence that is at least 85% identical to SEQ ID NO: 92.

In some embodiments, an H3N2 virus is A/Cambodia/e0826360/2020. In some embodiments, an HA polypeptide associated with A/Cambodia/e0826360/2020 comprises a sequence that is at least 85% identical to SEQ ID NO: 95. In some embodiments, an HA polypeptide associated with A/Cambodia/e0826360/2020 is encoded by an RNA comprising a sequence that is at least 85% identical to SEQ ID NO: 97.

In some embodiments, a B/Victoria lineage influenza virus is B/Washington/02/2019. In some embodiments, an HA polypeptide associated with B/Washington/02/2019 comprises an amino acid sequence that is at least 85% identical to SEQ ID NO: 100. In some embodiments, an HA polypeptide associated with B/Washington/02/2019 is encoded by an RNA comprising a nucleotide sequence that is at least 85% identical to SEQ ID NO: 102.

In some embodiments, a B/Yamagata lineage influenza virus is B/Phuket/3073/2013. In some embodiments, an HA polypeptide associated with B/Phuket/3073/2013 comprises an amino acid sequence that is at least 85% identical to SEQ ID NO: 105. In some embodiments, an HA polypeptide associated with B/Phuket/3073/2013 is encoded by an RNA comprising a sequence that is at least 85% identical to SEQ ID NO: 107.

In some embodiments, an S polypeptide associated with a Wuhan strain comprises a sequence that is at least 85% identical to SEQ ID NO: 7.

In some embodiments, an S polypeptide associated with a Wuhan strain comprises a sequence that is at least 85% identical to SEQ ID NO: 9.

In some embodiments, an Omicron variant is a BA.4/5 variant. In some embodiments, an S polypeptide associated with a BA.4/5 Omicron variant comprises a sequence that is at least 85% identical to SEQ ID NO: 69. In some embodiments, an S polypeptide associated with a BA.4/5 Omicron variant is encoded by an RNA that comprises a sequence that is at least 85% identical to SEQ ID NO: 70.

In some embodiments, an Omicron variant is an XBB.1.5 variant. In some embodiments, an S polypeptide associated with an XBB.1.5 Omicron variant comprises a sequence that is at least 85% identical to SEQ ID NO: 69.

In some embodiments, an S polypeptide associated with an XBB.1.5 variant is encoded by an RNA that comprises a sequence that is at least 85% identical to SEQ ID NO: 130.

In some embodiments, each of the RNAs in a composition disclosed herein comprises the same non-coding elements, wherein at least one of the non-coding elements is or comprises:
(i) a 5′-UTR sequence that is or comprises a modified human alpha-globin 5′-UTR;
(ii) a 3′-UTR sequence that is or comprises a first sequence from the amino terminal enhancer of split (AES) messenger RNA and a second sequence from the mitochondrial encoded 12S ribosomal RNA;
(iii) a polyA sequence comprising at least 100 A nucleotides, wherein the first RNA and the second RNA each do not comprise a stretch of at least 30 contiguous C nucleotides between the 3′ UTR and the polyA sequence;
(iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
(v) a 5′ cap comprising a Cap1 structure and a cap proximal sequence comprising positions +1, +2, +3, +4, and +5 of the RNA; wherein:
   (a) the Cap1 structure comprises m7(3′OMeG)(5′)ppp(5′)(2′OMeA₁)pG₂, wherein A₁ is position +1 of the RNA, and G₂ is position +2 of the RNA; and
   (b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

In some embodiments, each RNA in a composition comprises the same non-coding elements, wherein at least one of the same non-coding elements is or comprises:
(i) a 5′-UTR sequence that is or comprises a modified human alpha-globin 5′-UTR;
(ii) a 3′-UTR sequence that is or comprises a first sequence from the amino terminal enhancer of split (AES) messenger RNA and a second sequence from the mitochondrial encoded 12S ribosomal RNA;
(iii) a polyA sequence comprising at least 100 A nucleotides, wherein the first RNA and the second RNA each do not comprise a stretch of at least 30 contiguous C nucleotides between the 3′ UTR and the polyA sequence;
(iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; and
(v) a 5′ cap comprising a Cap1 structure and a cap proximal sequence comprising positions +1, +2, +3, +4, and +5 of the RNA; wherein:
   (a) the Cap1 structure comprises m7(3′OMeG)(5′)ppp(5′)(2′OMeA₁)pG₂, wherein A₁ is position +1 of the RNA, and G₂ is position +2 of the RNA; and
   (b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

In some embodiments, each RNA in a composition comprises, in 5′ to 3′ orientation, a 5′ cap, cap proximal sequence, 5′ UTR sequence, 3′ UTR sequence, and polyA sequence.

In some embodiments, each RNA in a composition comprises a 5′-cap that is or comprises m₂^{7,3′-O}Gppp(m₁^{2′-O})ApG.

In some embodiments, each RNA in a composition comprises a 5′ UTR that comprises or consists of a human alpha-globin 5′-UTR. In some embodiments, a human alpha-globin 5′-UTR comprises SEQ ID NO: 12.

In some embodiments, each RNA in a composition comprises a 3′ UTR that comprises or consists of a first sequence from the amino terminal enhancer of split (AES) messenger RNA and a second sequence from the mitochondrial encoded 12S ribosomal RNA. In some embodiments, each RNA in a composition comprises a 3′ UTR that comprises or consists of a sequence according to SEQ ID NO: 13.

In some embodiments, each RNA in a composition comprises a polyA tail sequence that is a interrupted polyA tail sequence. In some embodiments, an interrupted polyA tail sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence. In some embodiments, an interrupted polyA tail sequence comprises or consists of a ribonucleic acid sequence according to SEQ ID NO: 14.

In some embodiments, each RNA in a composition includes modified uridines in place of all uridines. In some embodiments, a modified uridines is N1-methyl-pseudouridine.

In some embodiments, a composition comprises one or more first RNAs and one or more second RNAs in a mass ratio of 1:5 to 5:1, 1:4 to 4:1, 1:3 to 3:1, or 1:2 to 2:1.

In some embodiments, each RNA in a composition is formulated in nanoparticles.

In some embodiments, all first RNAs in a composition are co-formulated together in the same population of nanoparticles and all second RNAs in a composition are co-formulated together in the same population of nanoparticles, wherein the first RNAs and the second RNAs are formulated in separate populations of nanoparticles.

In some embodiments, all first RNAs and all second RNAs in a composition are co-formulated together in the same population of nanoparticles.

In some embodiments, nanoparticles comprise lipid nanoparticles, polyplexes (PLX), lipidated polyplexes (LPLX), liposomes, or polysaccharide nanoparticles. In some embodiments, nanoparticles comprise lipid nanoparticles. In some embodiments, lipid nanoparticles comprise: a cationically ionizable lipid; and one or more neutral lipids, and a polymer-conjugated lipid. In some embodiments, a polymer-conjugated lipid comprises a PEG-conjugated lipid.

In some embodiments, nanoparticles have an average diameter of about 50-150 nm.

In some embodiments, compositions disclosed herein further comprise one or more third RNAs, each comprising a nucleotide sequence that encodes one or more antigenic polypeptides associated with a third infectious agent that is different from a first infectious agent and a second infectious agent. In some embodiments, compositions disclosed herein further comprise one or more antigenic polypeptides associated with a third infectious agent that is different from a first infectious agent and a second infectious agent. In some embodiments, the third infectious agent is a respiratory virus (e.g., a respiratory virus that is not a SARS-CoV-2 virus or an influenza virus). In some embodiments, the third infectious agent is respiratory syncytial virus (RSV).

In some embodiments, a composition comprises one or more RNAs, each encoding an RSV polypeptide. In some embodiments, a composition comprises one or more RSV polypeptides. In some embodiments, a composition comprises one or more RNAs, each encoding an RSV F protein, a variant thereof, or an immunogenic fragment of an RSV F protein or a variant thereof. In some embodiments, a composition comprises one or more RSV F proteins, an immunogenic variant thereof, or an immunogenic fragment of an RSV F protein or a variant thereof.

In some embodiments, a composition described herein comprises:
(i) one or more RNAs, each encoding a polypeptide of an RSV subtype A virus (e.g., an F protein, a variant thereof, or an immunogenic fragment of an F protein or a variant thereof), and one or more RNAs, each encoding a polypeptide of an RSV subtype B virus (e.g., an F protein, a variant thereof, or an immunogenic fragment of an F protein or a variant thereof); or
(ii) one or more polypeptides of an RSV subtype A virus (e.g., an F protein, a variant thereof, or an immunogenic fragment of an F protein or a variant thereof) and one or more polypeptides of an RSV subtype B virus (e.g., an F protein, a variant thereof, or an immunogenic fragment of an F protein or a variant thereof).

In some embodiments, an RSV F protein, variant, or immunogenic fragment is stabilized in a prefusion confirmation. In some embodiments, a composition comprises or describes RSVpreF (also known as Abrysvo™) and Arexvy™.

In some embodiments, the present disclosure provides a pharmaceutical composition comprising a composition disclosed herein and at least one pharmaceutically acceptable excipient. In some embodiments, a pharmaceutical composition comprises a cryoprotectant, optionally wherein the cryoprotectant is or comprises sucrose. In some embodiments, a pharmaceutical composition comprises an aqueous buffered solution, optionally wherein the aqueous buffered solution comprises one or more of Tris base, Tris HCl, NaCl, KCl, Na₂HPO₄, and KH₂PO₄.

In some embodiments a pharmaceutical composition is formulated to provide a dose of 100 μg or less of total RNA. In some embodiments, a pharmaceutical composition is formulated to provide a dose of 90 μg of total RNA. In some embodiments, a pharmaceutical composition is formulated to provide a dose of 60 μg of total RNA. In some embodiments, a pharmaceutical composition is formulated to provide a dose of 30 μg of one or more first RNAs and a dose of 60 μg of one or more second RNAs. In some embodiments, a pharmaceutical composition is formulated to provide a dose of 60 μg of one or more first RNAs and a dose of 30 μg of one or more second RNAs. In some embodiments, a pharmaceutical composition is formulated to provide a dose of 30 μg of one or more first RNAs and a dose of 30 μg of one or more second RNAs.

In some embodiments, a pharmaceutical composition comprises four second RNAs, each comprising a nucleotide sequence that encodes an antigenic polypeptide associated with a different influenza antigen, and wherein the pharmaceutical composition is formulated to provide a dose of 15 μg of each second RNA.

In some embodiments, a pharmaceutical composition comprises four second RNAs, each comprising a nucleotide sequence that encodes an antigenic polypeptide associated with a different influenza antigen, and wherein the pharmaceutical composition is formulated to provide a dose of 7.5 μg of each second RNA.

In some embodiments, a pharmaceutical composition comprises two first RNAs, each comprising a nucleotide sequence that encodes an antigenic polypeptide associated with a different coronavirus antigen, and wherein the pharmaceutical composition is formulated to provide a dose of 15 μg of each first RNA.

In some embodiments, a pharmaceutical composition comprises two first RNAs, each comprising a nucleotide sequence that encodes an antigenic polypeptide associated with a different coronavirus antigen, and wherein the pharmaceutical composition is formulated to provide a dose of 30 μg of each first RNA.

In some embodiments, disclosed herein is a method that comprises administering to a subject a composition or a pharmaceutical composition disclosed herein.

In some embodiments, disclosed herein is a method that comprises one or more doses of a pharmaceutical composition disclosed herein to a subject.

In some embodiments, a method disclosed herein is a method for treating coronavirus disease and influenza disease. In some embodiments, a method disclosed herein is a method for treating coronavirus disease and RSV disease. In some embodiments, a method disclosed herein is a method for treating coronavirus disease, influenza disease and RSV disease.

In some embodiments, a method disclosed herein is a method of preventing coronavirus disease and influenza disease. In some embodiments, a method disclosed herein is a method for preventing coronavirus disease and RSV disease. In some embodiments, a method disclosed herein is a method for preventing coronavirus disease, influenza disease and RSV disease.

In some embodiments, a method disclosed herein is a method of inducing an immune response against one or more coronaviruses and one or more influenza viruses. In some embodiments, a method disclosed herein is a method for inducing an immune response against one or more coronaviruses and ones or more RSVs. In some embodiments, a method disclosed herein is a method for inducing an immune response against one or more coronaviruses, one or more influenza viruses and one or more RSVs.

In some embodiments, one or more doses of a composition or a pharmaceutical composition is co-administered with a vaccine against a third infectious agent. In some embodiments, the third infectious agent is a virus that can cause a respiratory disease. In some embodiments, the third infectious agent is RSV. In some embodiments, the vaccine against a third infectious agent is Arexvy™ or Abrysvo™.

In some embodiments, a vaccine against a third infectious agent is mixed with one or more doses of a composition the one or more doses of a pharmaceutical compositions described herein immediately before administering to a subject. In some embodiments, a vaccine against a third infectious agent is administered separately from one or more doses of the composition or the one or more doses of the pharmaceutical compositions (e.g., wherein the vaccine against the third infectious agent and the one or more doses of the composition or the one or more doses of the pharmaceutical composition are administered to the subject at separate injection sites (e.g., on opposite arms).

In some embodiments, disclosed herein is a composition or pharmaceutical composition for use in treating a coronavirus disease, an influenza disease, and/or an RSV disease (e.g., a coronavirus disease and an influenza disease; a coronavirus disease and an RSV disease; or a coronavirus disease, an influenza disease, and an RSV disease) comprising administering one or more doses of the composition or pharmaceutical composition to a subject. In some embodiments, disclosed herein is a composition or pharmaceutical composition for use in the prevention of coronavirus disease, RSV disease, and/or influenza disease (e.g., coronavirus disease and influenza disease; coronavirus disease and RSV disease; or coronavirus disease, influenza disease, and RSV disease), wherein the use comprises administering one or more doses of the composition pharmaceutical composition to a subject. In some embodiments, the use comprises administering two or more doses of the composition or pharmaceutical composition. In some embodiments, the two or more doses are administered at least about 21 days apart.

In some embodiments a method or use disclosed herein comprises administering three or more doses of a composition or a pharmaceutical composition to a subject.

In some embodiments, a method or use comprises administering to a subject who has previously been exposed to a coronavirus and/or an influenza virus (e.g., by vaccination or by infection).

In some embodiments, a method or use induces an immune response in a subject against one or coronaviruses, one or more RSVs, and/or one or more influenza viruses (e.g., one or more coronaviruses and one or more influenza viruses; one or more coronaviruses and one or more RSVs; or one or more coronaviruses, one or more influenza viruses, and one or more RSVs). In some embodiments, an immune response comprises a B-cell response. In some embodiments, a B-cell response comprises production of antibodies directed against the one or more antigens. In some embodiments, an immune response comprises a T cell response. In some embodiments, a T-cell response is or comprises a CD4+ T cell response. In some embodiments, a T-cell response is or comprises a CD8+ T cell response.

In some embodiments, disclosed herein is a method or a use of a pharmaceutical composition disclosed herein treatment of a coronavirus disease, an RSV disease, and/or an influenza disease (e.g., a coronavirus disease and an influenza disease; a coronavirus disease and an RSV disease; or a coronavirus disease, an influenza disease, and an RSV disease).

In some embodiments, disclosed herein is a composition (e.g., a composition described herein) or a pharmaceutical composition (e.g., a pharmaceutical composition described herein) for use in preventing a coronavirus disease, an RSV disease, and/an influenza disease (e.g., a coronavirus disease and an influenza disease; a coronavirus disease and an RSV disease; or a coronavirus disease, an influenza disease, and an RSV disease). In some embodiments, disclosed herein is a composition or pharmaceutical composition for use in inducing an immune response in a subject against one or more coronaviruses, and one or more RSVs, and one or more influenza viruses (e.g., one or more coronaviruses and one or more influenza viruses; one or more coronaviruses and one or more RSVs; or one or more coronaviruses, one or more influenza viruses, and one or more RSVs).

In some embodiments, a composition comprises:
one or more RNAs, each encoding a polypeptide of a first infectious agent; and
one or more polypeptides of a second infectious agent.

In some embodiments, a composition comprises one or more RNAs, each encoding a polypeptide of a coronavirus (e.g., a SARS-CoV-2 virus). In some embodiments, a composition comprises one or more RNAs, each encoding a SARS-CoV-2 S protein, a variant thereof, or an immunogenic fragment of a SARS-CoV-2 S protein or variant thereof. In some embodiments, a composition comprises one or more RNAs, each encoding a SARS-CoV-2 S protein, a variant thereof, or an immunogenic fragment of a SARS-CoV-2 S protein or variant thereof of a Wuhan strain or a SARS-CoV-2 variant (e.g., an Omicron variant (e.g., an Omicron BA.1, BA.2, BA.4/5, or an XBB.1.5 variant (e.g., an RNA described herein))). In some embodiments, a composition comprises one or more polypeptides of an influenza virus. In some embodiments, a composition comprises one or more polypeptides of one or more influenza viruses (e.g., one or more polypeptides of two or more influenza virus strains (e.g., one or more polypeptides of four or more influenza virus strains that are prevalent or which have been predicted to be prevalent in a relevant jurisdiction)). In some embodiments, a composition comprises a commercially available influenza virus (e.g., a recombinant commercially available influenza virus, or an inactivated virus vaccine described herein). In some embodiments, a commercially available influenza virus is Flublok or Fluzone. In some embodiments, a composition comprises one or more polypeptides of an RSV. In some embodiments, a composition comprises one or more polypeptides associated with a first RSV subtype and one or more polypeptides of a second RSV subtype. In some embodiments, a composition comprises one or more RSV F proteins, variants thereof, or immunogenic fragments of RSV F proteins or variants thereof. In some embodiments, a composition comprises an RSV F protein or an immunogenic fragment thereof comprising one or more mutations that stabilize a prefusion confirmation of the F protein. In some embodiments, a composition comprises Arexvy™ or ABRYSVO™. In some embodiments, a composition comprises one or more polypeptides of a third infectious agent.

In some embodiments, a composition comprises:
one or more RNAs, each encoding one or more polypeptides of a coronavirus (e.g., a SARS-CoV-2 S protein, a variant thereof, or an immunogenic fragment of either of the foregoing;
one or more polypeptides of one or more influenza viruses; and
one or more polypeptides of one or more RSVs.

In some embodiments, a composition comprises
an RNA encoding a SARS-CoV-2 S protein of an Omicron variant (e.g., an RNA encoding an S protein of an Omicron BA.1, BA.4/5, or XBB.1.5 variant described herein);
a recombinant influenza vaccine (e.g., as described herein (e.g., a FluBlok vaccine)) or an inactivated virus vaccine (e.g., as described herein (e.g., Fluzone)); and
an RSV vaccine comprising a prefusion-stabilized F protein or an immunogenic fragment thereof (e.g., an RSV vaccine described herein (e.g., Arexvy™ or ABRYSVO™)).

In some embodiments, described herein is a combination comprising a SARS-CoV-2 vaccine comprising one or more mRNAs encoding a prefusion stabilized SARS-CoV-2 spike protein or a variant thereof; and
(a) an influenza vaccine comprising (i) one or more mRNAs encoding an HA protein of an influenza virus, or (ii) one or HA polypeptides, and/or
(b) an RSV vaccine comprising one or more prefusion stabilized RSV F proteins, or immunogenic fragments thereof.

In some embodiments, a combination comprises one or more mRNAs encoding a prefusion stabilized SARS-CoV-2 spike protein or a variant thereof, wherein the one or more mRNAs is formulated as an LNP.

In some embodiments, a composition comprises one or more mRNAs encoding an HA protein of an influenza virus, wherein the one or more mRNAs is formulated as an LNP.

In some embodiments, a combination comprises (1) a SARS-CoV-2 vaccine comprising one or more mRNAs encoding a prefusion stabilized SARS-CoV-2 spike protein or a variant thereof, and (2) an influenza vaccine or an RSV vaccine, wherein the (1) SARS-CoV-2 vaccine and the (2) influenza vaccine or RSV vaccine are provided in separate containers (e.g., vials or syringes). In some embodiments, a combination comprises (1) a SARS-CoV-2 vaccine comprising one or more mRNAs encoding a prefusion stabilized SARS-CoV-2 spike protein or a variant thereof, and (2) an influenza vaccine or an RSV vaccine, wherein the (1) SARS-CoV-2 vaccine and the (2) influenza vaccine or RSV vaccine are provided in a single container (e.g., vial or syringe).

In some embodiments, a combination comprises a SARS-CoV-2 vaccine, an influenza vaccine, and an RSV vaccine. In some embodiments, a combination comprises a SARS-CoV-2 vaccine, an influenza vaccine, and an RSV vaccine, all of which are provided in a single container (e.g., a vial or syringe). In some embodiments, a combination comprises a SARS-CoV-2 vaccine, an influenza vaccine, and an RSV vaccine, each of which is provided in a separate container (e.g., separate vials and/or syringes).

In some embodiments, a combination comprises:
(a) a SARS-CoV-2 vaccine and an influenza vaccine provided in a single container, and an RSV vaccine is provided in a separate container; or
(b) a SARS-CoV-2 vaccine and an RSV vaccine provided in a single container, and an influenza vaccine is provided in a separate container.

In some embodiments, a combination comprises a SARS-CoV-2 vaccine that is BNT162b2 (e.g., a monovalent or bivalent vaccine described herein).

In some embodiments, a combination comprises an influenza vaccine that is a recombinant influenza vaccine (e.g., as described herein (e.g., a FluBlok vaccine)); or comprises an inactivated influenza virus (e.g., Fluzone). In some embodiments, a combination comprises an RSV vaccine that comprises a prefusion-stabilized F protein or an immunogenic fragment thereof (e.g., an RSV vaccine described herein (e.g., RSVpreF or ABRYSVO™)).

In some embodiments, disclosed herein is a method for inducing an immune response against a first infectious agent and a second infectious agent, wherein the method comprises administering (i) a first nanoparticle formulated RNA comprising a nucleotide sequence encoding an antigenic polypeptide associated with a first infectious agent and (ii) a second LNP formulated RNA comprising a nucleotide sequence encoding an antigenic polypeptide associated with a second infectious agent,
wherein the immune response induced against each of the first and the second infectious agents is greater than the immune response induced when the LNPs are administered separately.

In some embodiments, disclosed herein is a method for reducing the amount of a first LNP-formulated RNA required to produce an immune response against a first infectious agent, wherein the RNA of the first LNP-formulated RNA comprises a nucleotide sequence encoding one or more antigenic polypeptides associated with a first infectious agent,
wherein the method comprises co-administering a second LNP-formulated RNA comprising a nucleotide sequence encoding one or more antigenic polypeptides associated with a second infectious agent, and
wherein the first infectious agent differs from the second infectious agent.

Also provided herein, inter alia, is a vessel comprising a recently admixed combination comprising: a SARS-CoV-2 vaccine and an influenza vaccine; a SARS-CoV-2 vaccine and an RSV vaccine; or a SARS-CoV-2 vaccine, an influenza vaccine, and an RSV vaccine.

In some embodiments, a vessel comprises a recently admixed combination comprising:
(a) a SARS-CoV-2 vaccine; and
(b) an influenza vaccine;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the influenza vaccine: (i) is a nanoparticle (e.g., LNP) formulated RNA vaccine, or (ii) comprises one or more antigenic polypeptides (e.g., an HA protein) of one or more influenza virus strains.

In some embodiments a vessel comprises a recently admixed combination comprising:
(a) a SARS-CoV-2 vaccine; and
(b) an RSV vaccine;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains.

In some embodiments, a vessel comprises a recently admixed combination comprising:
(a) a SARS-CoV-2 vaccine;
(b) an RSV vaccine;
(c) an influenza vaccine;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains; and
wherein the influenza vaccine: (i) is a nanoparticle (e.g., LNP) formulated RNA vaccine, or (ii) comprises one or more antigenic polypeptides (e.g., an HA protein) of one or more influenza virus strains.

In some embodiments, a vessel comprises a SARS-CoV-2 vaccine that is a monovalent or bivalent vaccine.

In some embodiments, a vessel comprises an influenza vaccine, wherein the influenza vaccine is a quadrivalent vaccine.

In some embodiments, a vessel comprises an influenza vaccine that is an inactivated influenza virus, a recombinant influenza vaccine, a live attenuated influenza vaccine, a non-adjuvanted influenza vaccine, an adjuvanted influenza vaccine, or a subunit or split vaccine.

In some embodiments, a vessel comprises an RSV vaccine that comprises a prefusion-stabilized F protein or an immunogenic fragment thereof of one or more RSV strains.

In some embodiments, a vessel is a syringe or a vial.

Also provided herein, inter alia, is a method of simultaneously vaccinating a human subject against each of SARS-CoV-2 and influenza, each of SARS-CoV-2 and RSV, or each of SARS-CoV-2, influenza, and RSV.

In some embodiments, a method of simultaneously vaccinating a human subject against each of SARS-CoV-2 and influenza comprises:
simultaneously administering a SARS-CoV-2 vaccine composition and an influenza vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the influenza vaccine: (i) is a nanoparticle (e.g., LNP) formulated RNA vaccine, or (ii) comprises one or more antigenic polypeptides (e.g., an HA protein) of one or more influenza virus strains.

In some embodiments, a method of simultaneously vaccinating a human subject against each of SARS-CoV-2 and RSV comprises:
simultaneously administering a SARS-CoV-2 vaccine composition and an RSV vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains.

In some embodiments, a method of simultaneously vaccinating a human subject against each of SARS-CoV-2, influenza, and RSV comprises:
simultaneously administering a SARS-CoV-2 vaccine composition, an influenza vaccine composition, and an RSV vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs));
wherein the influenza vaccine: (i) is a nanoparticle (e.g., LNP) formulated RNA vaccine, or (ii) comprises one or more antigenic polypeptides (e.g., an HA protein) of one or more influenza virus strains; and
wherein the RSV vaccine comprises one or more antigenic polypeptides (e.g., an F protein or an immunogenic fragment thereof) associated with one or more RSV strains.

In some embodiments, a method of simultaneously vaccinating against each of SARS-CoV-2 and influenza comprises a step of administering that comprises injecting a composition through a needle or port; and
wherein the injected composition includes both the SARS-CoV-2 vaccine composition and the influenza vaccine composition; and
wherein the SARS-CoV-2 vaccine composition and the influenza vaccine composition are optionally administered using a syringe (e.g., a dual chamber syringe).

In some embodiments, a method of simultaneously vaccinating against each of SARS-CoV-2 and RSV comprises a step of administering that comprises injecting a composition through a needle or port;
wherein the injected composition includes both the SARS-CoV-2 vaccine composition and the RSV vaccine composition; and
wherein the SARS-CoV-2 vaccine composition and the RSV vaccine composition are optionally administered using a syringe (e.g., a dual chamber syringe).

In some embodiments, a method of simultaneously vaccinating against each of SARS-CoV-2, and RSV comprises a step of administering that comprises injecting a composition through a needle or port;
wherein the injected composition includes each of the SARS-CoV-2 vaccine composition, the influenza vaccine composition, and the RSV vaccine composition; and
wherein the SARS-CoV-2 vaccine composition, the RSV vaccine composition, and the influenza vaccine composition are optionally administered using a syringe (e.g., a dual chamber syringe).

In some embodiments, a method of simultaneously vaccinating against each of SARS-CoV-2 and influenza further comprises a step, prior to a step of administering, of admixing a SARS-CoV-2 vaccine composition and a influenza vaccine composition.

In some embodiments, a method of simultaneously vaccinating against both SARS-CoV-2 and RSV comprises a step, prior to a step of administering, of admixing a SARS-CoV-2 vaccine composition and a RSV vaccine composition.

In some embodiments, a method of simultaneously vaccinating against each of SARS-CoV-2, influenza, and RSV comprises a step, prior to administering, of admixing a SARS-CoV-2 vaccine composition, a influenza vaccine composition, and a RSV vaccine composition.

In some embodiments, a step of admixing is performed within a period of time of before administering, which period of time is not more than 2 hours (e.g., not more than 1 hour, 30 minutes, 20 minutes, 15 minutes, 10 minutes, or 5 minutes).

In some embodiments, a vessel comprises or a method of simultaneously vaccinating uses a SARS-CoV-2 vaccine, wherein the SARS-CoV-2 vaccine composition comprises two or more RNAs, each encoding an S protein of a different SARS-CoV-2 strain or variant, and wherein the two or more RNAs are encapsulated in separate populations of LNPs.

In some embodiments, a vessel comprises or a method of simultaneously vaccinating uses an influenza vaccine that comprises two or more RNAs (e.g., four RNAs), each encoding an antigenic polypeptide (e.g., HA protein) of a different influenza strain, and wherein the two or more RNAs are encapsulated in separate populations of LNPs.

In some embodiments, a vessel comprises or a method of simultaneously administering uses a SARS-CoV-2 vaccine, wherein the SARS-CoV-2 vaccine comprises:
(a) (i) an RNA comprising a nucleotide sequence that includes modified uridines and encodes a first SARS-CoV-2 Spike (S) polypeptide from a SARS-CoV-2 strain, wherein the RNA encodes a polypeptide comprising an amino acid sequence that is at least 85% identical to SEQ ID NO: 7, and/or comprises a nucleotide sequence that is at least 85% identical to SEQ ID NO: 20 and/or a nucleotide sequence that is at least 85% identical to SEQ ID NO: 9, and (ii) an RNA comprising a nucleotide sequence that includes modified uridines and encodes a S polypeptide from an Omicron BA.4/5 SARS-CoV-2 variant, wherein the RNA comprises a nucleotide sequence that encodes a polypeptide comprising a sequence that is at least 85% identical to SEQ ID NO: 69 and/or wherein the RNA comprises a nucleotide sequence that is at least 85% identical to SEQ ID NO: 72 and/or a nucleotide sequence that is at least 85% identical to SEQ ID NO: 70; or
(b) an RNA comprising a nucleotide sequence that includes modified uridines and encodes a SARS-CoV-2 Spike (S) polypeptide, wherein the RNA comprises a nucleotide sequence that encodes a polypeptide comprising a sequence that is at least 85% identical to SEQ ID NO: 129 and/or wherein the RNA comprises a nucleotide sequence that is at least 85% identical to SEQ ID NO: 132 and/or a nucleotide sequence that is at least 85% identical to SEQ ID NO: 130.

In some embodiments, a vessel comprises or a method of simultaneously administering uses a SARS-CoV-2 vaccine, wherein the SARS-CoV-2 vaccine comprises an influenza vaccine, wherein the influenza vaccine comprises:
(a) (i) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94 and/or a nucleotide sequence that is at least 85% identical to SEQ ID NO: 92; (ii) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99 and/or a nucleotide sequence that is at least 85% identical to SEQ ID NO: 97; (iii) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104 and/or a nucleotide sequence that is at least 85% identical to SEQ ID NO: 102; and (iv) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109 and/or a nucleotide sequence that is at least 85% identical to SEQ ID NO: 107; or
(b) (i) an RNA comprising a nucleotide sequence that includes modified uridines and encodes an influenza hemagglutinin antigen from an influenza A H1N1 strain, wherein the nucleotide sequence is at least 85% identical to SEQ ID NO: 92 and/or at least 85% identical to SEQ ID NO: 94; (ii) an RNA comprising a nucleotide sequence that includes modified uridines and encodes an influenza hemagglutinin antigen from an influenza A H3N2 strain, wherein the nucleotide sequence is at least 85% identical to SEQ ID NO: 82 and/or at least 85% identical to SEQ ID NO: 84; (iii) an RNA comprising a nucleotide sequence that includes modified uridines and encodes an influenza hemagglutinin antigen from an influenza B Victoria strain, wherein the nucleotide sequence is at least 85% identical to SEQ ID NO: 87 and/or that is at least 85% identical to SEQ ID NO: 89; and (iv) an RNA comprising a nucleotide sequence that includes modified uridines and encodes an influenza hemagglutinin antigen from an influenza B Yamagata strain, wherein the nucleotide sequence is at least 85% identical to SEQ ID NO: 107 and/or that is at least 85% identical to SEQ ID NO: 109.

SARS-CoV-2 is an RNA virus with four structural proteins. One of them, the spike protein is a surface protein which binds the angiotensin-converting enzyme 2 (ACE-2) present on host cells. Therefore, the spike protein is considered a relevant antigen for vaccine development.

BNT162b2 (which comprises an RNA comprising SEQ ID NO: 20) is an mRNA vaccine for prevention of COVID-19 and has demonstrated an efficacy of 95% or more at preventing COVID-19. The vaccine comprises a 5′capped mRNA encoding for the full-length SARS-CoV-2 spike glycoprotein (S) encapsulated in lipid nanoparticles (LNPs). The finished product is presented as a concentrate for dispersion for injection containing BNT162b2 as active substance. Other ingredients include: ALC-0315 (4-hydroxybutyl)azanediyl)bis(hexane-6,1-diyl)bis(2-hexyldecanoate), ALC-0159 (2-[(polyethylene glycol)-2000]-N,N-ditetradecylacetamide), 1,2-Distearoyl-sn-glycero-3-phosphocholine (DSPC), cholesterol, and in some embodiments, potassium chloride, potassium dihydrogen phosphate, sodium chloride, disodium phosphate dihydrate, sucrose and water for injection.

In some embodiments, a different buffer may be used in lieu of PBS. In some embodiments, BNT162b2 is formulated in a Tris-buffered solution, optionally comprising sucrose. In some embodiments, the formulation comprises ALC-0315 (4-hydroxybutyl)azanediyl)bis(hexane-6,1-diyl)bis(2-hexyldecanoate), ALC-0159 (2-[(polyethylene glycol)-2000]-N,N-ditetradecylacetamide), DSPC (1,2-distearoyl-sn-glycero-3-phosphocholine), cholesterol, sucrose, trometamol (Tris), trometamol hydrochloride and water.

In some embodiments, the concentration of RNA in a pharmaceutical RNA preparation is about 0.1-0.2 mg/ml. In some embodiments, the concentration of RNA in a pharmaceutical RNA preparation is about 0.1 mg/ml. In some embodiments, the concentration of RNA in a pharmaceutical RNA preparation is about 0.12 mg/ml. In some embodiments, the concentration of RNA in a pharmaceutical RNA preparation is about 0.14 mg/ml. In some embodiments, the concentration of RNA in a pharmaceutical RNA preparation is about 0.16 mg/ml. In some embodiments, the concentration of RNA in a pharmaceutical RNA preparation is about 0.18 mg/ml. In some embodiments about 30 ug of RNA is administered by administering about 200 uL of RNA preparation. In some embodiments, the RNA in a pharmaceutical RNA preparation is diluted prior to administration (e.g., diluted to a concentration of about 0.05 mg/ml). In some embodiments, administration volumes are between about 200 μl and about 300 μl. In some embodiments, the RNA in a pharmaceutical RNA preparation is formulated in about 10 mM Tris buffer, and about 10% sucrose.

In some embodiments, a pharmaceutical RNA preparation comprises RNA in a concentration of about 0.1 mg/ml, and is formulated in about 10 mM Tris buffer, and about 10% sucrose. In some embodiments, a pharmaceutical RNA preparation comprises RNA in a concentration of about 0.12 mg/ml, and is formulated in about 10 mM Tris buffer, and about 10% sucrose. In some embodiments, a pharmaceutical RNA preparation comprises RNA in a concentration of about 0.14 mg/ml, and is formulated in about 10 mM Tris buffer, and about 10% sucrose. In some embodiments, a pharmaceutical RNA preparation comprises RNA in a concentration of about 0.16 mg/ml, and is formulated in about 10 mM Tris buffer, and about 10% sucrose. In some embodiments, a pharmaceutical RNA preparation comprises RNA in a concentration of about 0.18 mg/ml, and is formulated in about 10 mM Tris buffer, and about 10% sucrose.

In some embodiments, formulations provided herein (e.g., formulations comprising about 10 mM Tris buffer and about 10% sucrose) can be can be diluted as needed prior to administration to administer different doses of RNA while keeping total injection volume relatively constant. For example, a dose of RNA of about 10 μg can be administered by diluting a pharmaceutical preparation comprising RNA in a concentration of about 0.1 mg/ml, and by about 1:1 and administering about 200 μl of diluted pharmaceutical RNA preparation.

In some embodiments, a vaccine is formulated in a vial (e.g., a glass vial). In some embodiments, a glass vial is sealed with a bromobutyl elastomeric stopper and an aluminum seal with flip-off plastic cap.

In some embodiments, a composition comprises an RNA comprising a nucleotide sequence encoding an antigenic polypeptide associated with a coronavirus. In some embodiments, the coronavirus is a betacoronavirus. In some embodiments, the betacoronavirus is SARS-CoV-2. In some embodiments, the antigenic polypeptide associated with a coronavirus is a Spike (S) protein (e.g., a SARS-CoV-2 S protein). The SARS-CoV-2 S protein encoded by BNT162b2 was chosen based on the sequence published at “SARS-CoV-2 isolate Wuhan-Hu-1”: GenBank: MN908947.3 (complete genome) and GenBank: QHD43416.1 (spike surface glycoprotein). In some embodiments, an RNA comprising a sequence encoding a SARS-CoV-2 S protein is a single-stranded, 5′-capped codon-optimized mRNA that is translated into the spike antigen of SARS-CoV-2. In some embodiments, an encoded spike antigen protein sequence contains two proline mutations, which stabilizes an antigenically improved, pre-fusion confirmation (P2 S). In some embodiments, RNA comprising a nucleotide sequence that encodes a SARS-CoV-2 S protein does not contain any uridines. In some embodiments, instead of uridine N1-methylpseudouridine is used in RNA synthesis. RNA encoding a SARS-CoV-2 S protein is translated into the SARS-CoV-2 S protein in a host cell. The S protein is then expressed on the cell surface where it induces an adaptive immune response. The S protein is identified as a target for neutralising antibodies against the virus and is considered a relevant vaccine component. The recent emergence of novel circulating variants of SARS-CoV-2 has raised significant concerns about geographic and temporal efficacy of vaccine interventions. One of the earliest variants that emerged and rapidly became globally dominant was D614G.

The alpha variant (also known as B.1.1.7, VOC202012/01, 501Y.V1 or GRY) was initially detected in the United Kingdom. The alpha variant has a large number of mutations, including several mutations in the S gene. It has been shown to be inherently more transmissible, with a growth rate that has been estimated to be 40-70% higher than other SARS-CoV-2 lineages in multiple countries (Volz et al., 2021, Nature, https_//doi.org/10.1038/s41586-021-03470-x; Washington et al., 2021, Cell https_//doi.org/10.1016/j.cell.2021.03.052).

The beta variant (also known as B.1.351 or GH/501Y.V2) was first detected in South Africa. The beta variant carries several mutations in the S gene. Three of these mutations are at sites in the RBD that are associated with immune evasion: N501Y (shared with alpha) and E484K and K417N.

The gamma variant (also known as P.1 or GR/501Y.V3) was first detected in Brazil. The gamma variant carries several mutations that affect the spike protein, including two shared with beta (N501Y and E484K), as well as a different mutation at position 417 (K417T).

The delta variant (also known as B.1.617.2 or G/478K.V1) was first documented in India. The delta variant has several point mutations that affect the spike protein, including P681R (a mutation position shared with alpha and adjacent to the furin cleavage site), and L452R, which is in the RBD and has been linked with increased binding to ACE2 and neutralizing antibody resistance. There is also a deletion in the spike protein at position 156/157.

These four VOCs have circulated globally and have become dominant variants in the geographic regions where they were first identified.

On 24 Nov. 2021, the Omicron (B.1.1.529) variant was first reported to WHO from South Africa. SARS-CoV-2 Omicron and its sublineages have had a major impact on the epidemiological landscape of the COVID-19 pandemic since initial emergence in November 2021 (WHO Technical Advisory Group on SARS-CoV-2 Virus Evolution (TAG-VE): Classification of Omicron (B.1.1.259): SARS-CoV-2 Variant of Concern (2021); WHO Headquarters (HQ), WHO Health Emergencies Programme, Enhancing Response to Omicron SARS-CoV-2 variant: Technical brief and priority actions for Member States (2022)). Significant alterations in the spike(S) glycoprotein of the first Omicron variant BA.1 leading to the loss of many neutralizing antibody epitopes (M. Hoffmann et al., “The Omicron variant is highly resistant against antibody mediated neutralization: Implications for control of the COVID-19 pandemic”, Cell 185, 447-456.e11 (2022)) rendered BA.1 capable of partially escaping previously established SARS-CoV-2 wild-type strain (Wuhan-Hu-1)-based immunity (V. Servellita, et al., “Neutralizing immunity in vaccine breakthrough infections from the SARS-CoV-2 Omicron and Delta variants”, Cell 185, 1539-1548.e5 (2022); Y. Cao et al., “Omicron escapes the majority of existing SARS-CoV-2 neutralizing antibodies”, Nature 602, 657-663 (2022)). Hence, breakthrough infection of vaccinated individuals with Omicron are more common than with previous Variants of Concern (VOCs). While Omicron BA.1 was displaced by the BA.2 variant in many countries around the globe, other variants such as BA.1.1 and BA.3 temporarily and/or locally gained momentum but did not become globally dominant (S. Xia et al., “Origin, virological features, immune evasion and intervention of SARS-CoV-2 Omicron sublineages. Signal Transduct. Target. Ther. 7, 241 (2022); H. Gruell et al., “SARS-CoV-2 Omicron sublineages exhibit distinct antibody escape patterns, Cell Host Microbe 7, 241 (2022).). Omicron BA.2.12.1 subsequently displaced BA.2 to become dominant in the United States, whereas BA.4 and BA.5 displaced BA.2 in Europe, parts of Africa, and Asia/Pacific (H. Gruell et al., “SARS-CoV-2 Omicron sublineages exhibit distinct antibody escape patterns,” Cell Host Microbe 7, 241 (2022); European Centre for Disease Prevention and Control, Weekly COVID-19 country overview-Country overview report: Week 31 2022 (2022); J. Hadfield et al., “Nextstrain: Real-time tracking of pathogen evolution,” Bioinformatics 34, 4121-4123 (2018)). Currently, Omicron BA.5 is dominant globally, including in the United States (Centers for Disease Control and Prevention. COVID Data Tracker. Atlanta, GA: US Department of Health and Human Services, CDC; 2022 Aug. 12. https_//covid.cdc.gov/coviddata-tracker (2022)).

Omicron has acquired numerous alterations (amino acid exchanges, insertions, or deletions) in the S glycoprotein, among which some are shared between all Omicron VOCs while others are specific to one or more Omicron sublineages. Antigenically, BA.2.12.1 exhibits high similarity with BA.2 but not BA.1, whereas BA.4 and BA.5 differ considerably from their ancestor BA.2 and even more so from BA.1, in line with their genealogy (A. Z. Mykytyn et al., “Antigenic cartography of SARS-CoV-2 reveals that Omicron BA.1 and BA.2 are antigenically distinct,” Sci. Immunol. 7, eabq4450 (2022).). Major differences of BA.1 from the remaining Omicron VOCs include Δ143-145, L212I, or ins214EPE in the S glycoprotein N-terminal domain and G446S or G496S in the receptor binding domain (RBD). Amino acid changes T376A, D405N, and R408S in the RBD are in turn common to BA.2 and its descendants but not found in BA.1. In addition, some alterations are specific for individual BA.2-descendant VOCs, including L452Q for BA.2.12.1 or L452R and F486V for BA.4 and BA.5 (BA.4 and BA.5 encode for the same S sequence). Most of these shared and VOC-specific alterations were shown to play an important role in immune escape from monoclonal antibodies and polyclonal sera raised against the wild-type S glycoprotein. In particular, the BA.4/BA.5-specific alterations are strongly implicated in immune escape of these VOCs (P. Wang et al., “Antibody resistance of SARS-CoV-2 variants B.1.351 and B.1.1.7. Nature 593, 130-135 (2021); Q. Wang et al., “Antibody evasion by SARS-CoV-2 Omicron subvariants BA.2.12.1, BA.4, & BA.5. Nature 608, 603-608 (2022)). As of the filing date of the present application, the XBB group of variants, resulting from a recombination of BA.10.1 and BA.2.75, are the most prevalent SARS-CoV-2 variants of concern, representing the top three most prevalent strains in the US between May 28, 2023, and Jun. 10, 2023.

Similarly, the ability of influenza to mutate and evade existing immune responses is well known. Influenza viruses are part of the Orthomyxoviridae family and are divided into 3 genera or types (A, B, and C) based upon antigenic differences in the nucleoprotein and the matrix protein. Influenza A viruses are further classified into subtypes based upon membrane glycoproteins, hemagglutinin (HA) and neuraminidase (NA) (Cox N J, Subbarao K. Influenza. Lancet. 1999; 354(9186):1277-82). Influenza A subtypes H1N1 (also written as A(H1N1)pdm09) and H3N2 are currently circulating in humans. H1N1 was responsible for the 2009 pandemic, and replaced the influenza A(H1N1) virus that had circulated prior to 2009. To date, all influenza pandemics have been caused by influenza type A viruses.

The RNA genome of influenza is segmented, which allows genetic reassortment among viruses of the same type. This genetic instability can result in the phenomenon known as antigenic shift, involving a major change in one or both of the HAs and NAs, which, if efficiently transmissible, can result in a pandemic. More common are multiple point mutations in the genome, leading to more minor changes in the HA and NA, known as antigenic drift (Hall E. Influenza. Chapter 12. In: Centers for Disease Control and Prevention. Hall E, Wodi A P, Hamborsky J, et al, eds. Epidemiology and prevention of vaccine-preventable diseases. 14th ed. Washington, DC: Public Health Foundation; 2021:179-92). Currently, two lineages of influenza B circulate, Victoria (B/Victoria) and Yamagata (B/Yamagata), based on differences in HA (Rota P A, Hemphill M L, Whistler T, Regnery H L, Kendal APJJoGV. Antigenic and genetic characterization of the haemagglutinins of recent cocirculating strains of influenza B virus. J General Virology. 1992; 73(10):2737-42). Both influenza A and B undergo genetic mutations, which are subject to selection pressure from human immune responses, leading to drift. This genetic instability is what necessitates current vaccines to be tailored annually to the influenza that are prevalent or predicted to be prevalent.

There is still a need for effective vaccine strategies against SARS-CoV-2 and influenza.

The present disclosure discloses technologies (e.g., compositions and methods) for inducing an immune response against multiple infectious agents. In particular, the present disclosure provides technologies for inducing an immune response against a coronavirus and one or more additional respiratory diseases (e.g., influenza). In some embodiments, compositions disclosed herein comprise RNA comprising a nucleotide sequence that encodes an amino acid of a SARS-CoV-2 S protein, an immunogenic variant thereof, or an immunogenic fragment of the SARS-CoV-2 S protein or the immunogenic variant thereof. In some embodiments, compositions disclosed herein comprise RNA comprising a nucleotide sequence encoding an HA protein from an influenza virus, or an immunogenic variant thereof, or an immunogenic fragment of the SARS-CoV-2 S protein or an immunogenic variant thereof. RNA encoding antigen polypeptide is administered to provide (following expression of the polynucleotide by appropriate target cells) antigen for induction, i.e., stimulation, priming and/or expansion, of an immune response, e.g., antibodies and/or immune effector cells, which is targeted to target antigen (e.g., a coronavirus S protein, in particular SARS-CoV-2 S protein and an influenza virus HA protein) or a procession product thereof. In one embodiment, the immune response which is to be induced according to the present disclosure is a B cell-mediated immune response, i.e., an antibody-mediated immune response. Additionally or alternatively, in one embodiment, the immune response which is to be induced according to the present disclosure is a T cell-mediated immune response. In one embodiment, the immune response is an anti-coronavirus, in particular anti-SARS-CoV-2 immune response. In one embodiment, the immune response is an anti-influenza virus immune response, in particular anti-subtype A and/or subtype B immune response.

In some embodiments, vaccines described herein comprise, as an active principle, one or more single-stranded RNAs that may be translated into the respective protein upon entering cells of a recipient. In addition to wildtype or codon-optimized sequences encoding the antigen sequence, RNA may contain one or more structural elements optimized for maximal efficacy with respect to stability and translational efficiency (e.g., 5′ cap, 5′ UTR, 3′ UTR, poly(A)-tail, or combinations thereof). In one embodiment, RNA described herein contains all of these elements. In one embodiment, a cap1 structure may be utilized as specific capping structure at the 5′-end of an RNA drug substance. In one embodiment, beta-S-ARCA(D1) (m₂^{7,2′-O}GppSpG) or m₂^{7,3′-O}Gppp(m₁^{2′-O})ApG may be utilized as specific capping structure at the 5′-end of an RNA drug substances. As 5′-UTR sequence, the 5′-UTR sequence of the human alpha-globin mRNA, optionally with an optimized ‘Kozak sequence’ to increase translational efficiency (e.g., SEQ ID NO: 12) may be used. As 3′-UTR sequence, a combination of two sequence elements (FI element) derived from the “amino terminal enhancer of split” (AES) mRNA (called F) and the mitochondrial encoded 12S ribosomal RNA (called I) (e.g., SEQ ID NO: 13) placed between the coding sequence and the poly(A)-tail to assure higher maximum protein levels and prolonged persistence of the mRNA may be used. These sequences were identified using an ex vivo selection process for sequences that confer RNA stability and augment total protein expression (see WO 2017/060314, herein incorporated by reference). Alternatively, the 3′-UTR may be two re-iterated 3′-UTRs of the human beta-globin mRNA. Additionally or alternatively, in some embodiments, a poly(A)-tail may comprise a length of at least 100 adenosine residues (SEQ ID NO: 180) (including, e.g., at least 110 adenosine residues, at least 120 adenosine residues, 130 adenosine residues, or longer). In some embodiments, a poly(A)-tail may comprise a length of about 100 to about 150 adenosine residues. In some embodiments a poly(A)-tail may comprise an interrupted poly(A)-tail. For example, in some such embodiments, a poly(A)-tail measuring 110 nucleotides in length, consisting of a stretch of 30 adenosine residues (SEQ ID NO: 174), followed by a 10 nucleotide linker sequence (of random nucleotides) and another 70 adenosine residues (SEQ ID NO: 175) (e.g., SEQ ID NO: 14) may be used. This poly(A)-tail sequence was designed to enhance RNA stability and translational efficiency.

In some embodiments, a secretory signal peptide (sec) can be fused to an antigen described herein (e.g., as an N terminal tag), or an RNA may comprise such an antigen fused to a sec. In one embodiment, see corresponds to the secretory signal peptide of the S protein and is fused to the N-terminus of an S protein. Sequences coding for short linker peptides predominantly consisting of the amino acids glycine (G) and serine(S), as commonly used for fusion proteins may be used as GS/Linkers between see and an antigen region.

In some embodiments, RNA described herein may be complexed with proteins and/or lipids, preferably lipids, to generate RNA-particles for administration. If a combination of different RNAs is used, the RNAs may be complexed together or complexed separately with proteins and/or lipids to generate RNA-particles for administration.

In one aspect, the present disclosure relates to a composition or medical preparation comprising RNA encoding an amino acid sequence comprising a SARS-CoV-2 S protein, an immunogenic variant thereof, or an immunogenic fragment of the SARS-CoV-2 S protein or the immunogenic variant thereof.

In one embodiment, an immunogenic fragment of the SARS-CoV-2 S protein comprises the S1 subunit of the SARS-CoV-2 S protein, or the receptor binding domain (RBD) of the S1 subunit of the SARS-CoV-2 S protein.

In one embodiment, the amino acid sequence comprising a SARS-CoV-2 S protein, an immunogenic variant thereof, or an immunogenic fragment of the SARS-CoV-2 S protein or the immunogenic variant thereof is able to form a multimeric complex, in particular a trimeric complex. To this end, an amino acid sequence comprising a SARS-CoV-2 S protein, an immunogenic variant thereof, or an immunogenic fragment of the SARS-CoV-2 S protein or the immunogenic variant thereof may comprise a domain allowing the formation of a multimeric complex, in particular a trimeric complex of the amino acid sequence comprising a SARS-CoV-2 S protein, an immunogenic variant thereof, or an immunogenic fragment of the SARS-CoV-2 S protein or the immunogenic variant thereof. In one embodiment, the domain allowing the formation of a multimeric complex comprises a trimerization domain, for example, a trimerization domain as described herein, e.g., SARS-CoV-2 S protein trimerization domain. In one embodiment, trimerization is achieved by addition of a trimerization domain, e.g., a T4-fibritin-derived “foldon” trimerization domain (e.g., SEQ ID NO: 10), in particular if the amino acid sequence comprising a SARS-CoV-2 S protein, an immunogenic variant thereof, or an immunogenic fragment of the SARS-CoV-2 S protein or the immunogenic variant thereof corresponds to a portion of a SARS-CoV-2 S protein that does not comprise the SARS-CoV-2 S protein trimerization domain.

In one embodiment, an amino acid sequence comprising a SARS-CoV-2 S protein, an immunogenic variant thereof, or an immunogenic fragment of the SARS-CoV-2 S protein or the immunogenic variant thereof is encoded by a coding sequence which is codon-optimized and/or the G/C content of which is increased compared to wild type coding sequence, wherein the codon-optimization and/or the increase in the G/C content preferably does not change the sequence of the encoded amino acid sequence. Those skilled in the art will appreciate that codon optimization involves choosing between or among alternative codons encoding the same amino acid residue. Codon optimization typically includes consideration of codon(s) preferred by a particular host in which a sequence is to be expressed.

In accordance with the present disclosure, in many embodiments, a preferred host is a human. In some embodiments, a preferred host may be a domestic animal. Alternatively or additionally, in some embodiments, selection between or among possible codons encoding the same amino acid may consider one or more other features such as, for example, overall G/C content (as noted above) and/or similarity to a particular reference. For example, in some embodiments of the present disclosure, a provided coding sequence that encodes a SARS-CoV-2 S protein or immunogenic variant thereof that differs in amino acid sequence from that encoded by a BNT162b2 construct described herein utilizes a codon, in at least one position of such difference, that preserves greater similarity to the BNT162b2 construct sequence relative to at least one alternative codon encoding the same amino acid at such position of difference.

In one embodiment, an RNA is a modified RNA, in particular a stabilized mRNA. In one embodiment, an RNA comprises a modified nucleoside in place of at least one uridine. In one embodiment, an RNA comprises a modified nucleoside in place of each uridine. In one embodiment, a modified nucleoside is independently selected from pseudouridine (ψ), N1-methyl-pseudouridine (m1ψ), and 5-methyl-uridine (m5U).

In one embodiment, an RNA comprises a modified nucleoside in place of uridine.

In one embodiment, the modified nucleoside is selected from pseudouridine (ψ), N1-methyl-pseudouridine (m1ψ), and 5-methyl-uridine (m5U).

In one embodiment, RNA comprises a 5′ cap.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 979 to 1584 of SEQ ID NO: 2, 8 or 9, a nucleotide sequence having at least 99%, 98%, 97%, 96%, 95%, 90%, 85%, or 80% identity to the nucleotide sequence of nucleotides 979 to 1584 of SEQ ID NO: 2, 8 or 9, or a fragment of the nucleotide sequence of nucleotides 979 to 1584 of SEQ ID NO: 2, 8 or 9, or the nucleotide sequence having at least 99%, 98%, 97%, 96%, 95%, 90%, 85%, or 80% identity to the nucleotide sequence of nucleotides 979 to 1584 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 327 to 528 of SEQ ID NO: 1, an amino acid sequence having at least 99%, 98%, 97%, 96%, 95%, 90%, 85%, or 80% identity to the amino acid sequence of amino acids 327 to 528 of SEQ ID NO: 1, or an immunogenic fragment of the amino acid sequence of amino acids 327 to 528 of SEQ ID NO: 1, or the amino acid sequence having at least 99%, 98%, 97%, 96%, 95%, 90%, 85%, or 80% identity to the amino acid sequence of amino acids 327 to 528 of SEQ ID NO: 1.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 111 to 986 of SEQ ID NO: 30, a nucleotide sequence having at least 99%, 98%, 97%, 96%, 95%, 90%, 85%, or 80% identity to the nucleotide sequence of nucleotides 111 to 986 of SEQ ID NO: 30, or a fragment of the nucleotide sequence of nucleotides 111 to 986 of SEQ ID NO: 30, or the nucleotide sequence having at least 99%, 98%, 97%, 96%, 95%, 90%, 85%, or 80% identity to the nucleotide sequence of nucleotides 111 to 986 of SEQ ID NO: 30; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 20 to 311 of SEQ ID NO: 29, an amino acid sequence having at least 99%, 98%, 97%, 96%, 95%, 90%, 85%, or 80% identity to the amino acid sequence of amino acids 20 to 311 of SEQ ID NO: 29, or an immunogenic fragment of the amino acid sequence of amino acids 20 to 311 of SEQ ID NO: 29, or the amino acid sequence having at least 99%, 98%, 97%, 96%, 95%, 90%, 85%, or 80% identity to the amino acid sequence of amino acids 20 to 311 of SEQ ID NO: 29.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 2055 of SEQ ID NO: 2, 8 or 9, a nucleotide sequence having at least 99%, 98%, 97%, 96%, 95%, 90%, 85%, or 80% identity to the nucleotide sequence of nucleotides 49 to 2055 of SEQ ID NO: 2, 8 or 9, or a fragment of the nucleotide sequence of nucleotides 49 to 2055 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2055 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 685 of SEQ ID NO: 1, an amino acid sequence having at least 99%, 98%, 97%, 96%, 95%, 90%, 85%, or 80% identity to the amino acid sequence of amino acids 17 to 685 of SEQ ID NO: 1, or an immunogenic fragment of the amino acid sequence of amino acids 17 to 685 of SEQ ID NO: 1, or the amino acid sequence having at least 99%, 98%, 97%, 96%, 95%, 90%, 85%, or 80% identity to the amino acid sequence of amino acids 17 to 685 of SEQ ID NO: 1.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 3819 of SEQ ID NO: 2, 8 or 9, a nucleotide sequence having at least 99%, 98%, 97%, 96%, 95%, 90%, 85%, or 80% identity to the nucleotide sequence of nucleotides 49 to 3819 of SEQ ID NO: 2, 8 or 9, or a fragment of the nucleotide sequence of nucleotides 49 to 3819 of SEQ ID NO: 2, 8 or 9, or the nucleotide sequence having at least 99%, 98%, 97%, 96%, 95%, 90%, 85%, or 80% identity to the nucleotide sequence of nucleotides 49 to 3819 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 1273 of SEQ ID NO: 1 or 7, an amino acid sequence having at least 99%, 98%, 97%, 96%, 95%, 90%, 85%, or 80% identity to the amino acid sequence of amino acids 17 to 1273 of SEQ ID NO: 1 or 7, or an immunogenic fragment of the amino acid sequence of amino acids 17 to 1273 of SEQ ID NO: 1 or 7, or the amino acid sequence having at least 99%, 98%, 97%, 96%, 95%, 90%, 85%, or 80% identity to the amino acid sequence of amino acids 17 to 1273 of SEQ ID NO: 1 or 7.

In one embodiment, the amino acid sequence comprising a SARS-CoV-2 S protein, an immunogenic variant thereof, or an immunogenic fragment of the SARS-CoV-2 S protein or the immunogenic variant thereof comprises a secretory signal peptide.

In one embodiment, the secretory signal peptide is fused, preferably N-terminally, to a SARS-CoV-2 S protein, an immunogenic variant thereof, or an immunogenic fragment of the SARS-CoV-2 S protein or the immunogenic variant thereof.

In one embodiment,
(i) the RNA encoding the secretory signal peptide comprises the nucleotide sequence of nucleotides 1 to 48 of SEQ ID NO: 2, 8 or 9, a nucleotide sequence having at least 99%, 98%, 97%, 96%, 95%, 90%, 85%, or 80% identity to the nucleotide sequence of nucleotides 1 to 48 of SEQ ID NO: 2, 8 or 9, or a fragment of the nucleotide sequence of nucleotides 1 to 48 of SEQ ID NO: 2, 8 or 9, or the nucleotide sequence having at least 99%, 98%, 97%, 96%, 95%, 90%, 85%, or 80% identity to the nucleotide sequence of nucleotides 1 to 48 of SEQ ID NO: 2, 8 or 9; and/or
(ii) the secretory signal peptide comprises the amino acid sequence of amino acids 1 to 16 of SEQ ID NO: 1, an amino acid sequence having at least 99%, 98%, 97%, 96%, 95%, 90%, 85%, or 80% identity to the amino acid sequence of amino acids 1 to 16 of SEQ ID NO: 1, or a functional fragment of the amino acid sequence of amino acids 1 to 16 of SEQ ID NO: 1, or the amino acid sequence having at least 99%, 98%, 97%, 96%, 95%, 90%, 85%, or 80% identity to the amino acid sequence of amino acids 1 to 16 of SEQ ID NO: 1.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6, or the nucleotide sequence having at least 99%, 98%, 97%, 96%, 95%, 90%, 85%, or 80% identity to the nucleotide sequence of SEQ ID NO: 6; and/or
(ii) a SARS-CoV-2 S protein, an immunogenic variant thereof, or an immunogenic fragment of the SARS-CoV-2 S protein or the immunogenic variant thereof comprises the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 54 to 986 of SEQ ID NO: 30, a nucleotide sequence having at least 99%, 98%, 97%, 96%, 95%, 90%, 85%, or 80% identity to the nucleotide sequence of nucleotides 54 to 986 of SEQ ID NO: 30, or a fragment of the nucleotide sequence of nucleotides 54 to 986 of SEQ ID NO: 30, or the nucleotide sequence having at least 99%, 98%, 97%, 96%, 95%, 90%, 85%, or 80% identity to the nucleotide sequence of nucleotides 54 to 986 of SEQ ID NO: 30; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 1 to 311 of SEQ ID NO: 29, an amino acid sequence having at least 99%, 98%, 97%, 96%, 95%, 90%, 85%, or 80% identity to the amino acid sequence of amino acids 1 to 311 of SEQ ID NO: 29, or an immunogenic fragment of the amino acid sequence of amino acids 1 to 311 of SEQ ID NO: 29, or the amino acid sequence having at least 99%, 98%, 97%, 96%, 95%, 90%, 85%, or 80% identity to the amino acid sequence of amino acids 1 to 311 of SEQ ID NO: 29.

In one embodiment, each RNA in a composition is a modified RNA, in particular a stabilized mRNA. In one embodiment, each RNA in a composition comprises a modified nucleoside in place of at least one uridine. In one embodiment, each RNA in a composition comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, each RNA in a composition comprises a modified nucleoside in place of uridine.

In one embodiment, the modified nucleoside is selected from pseudouridine (ψ), N1-methyl-pseudouridine (m1ψ), and 5-methyl-uridine (m5U).

In one embodiment, each RNA in a composition comprises a 5′ cap.

In one embodiment, each RNA in a composition comprises a 5′ UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In one embodiment, each RNA in a composition comprises a 3′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In one embodiment, each RNA in a composition comprises a poly-A sequence.

In one embodiment, the poly-A sequence comprises at least 100 nucleotides.

In one embodiment, the poly-A sequence comprises or consists of the nucleotide sequence of SEQ ID NO: 14.

In one embodiment, each RNA in a composition is formulated or is to be formulated as a liquid, a solid, or a combination thereof.

In one embodiment, each RNA in a composition is formulated or is to be formulated for injection.

In one embodiment, each RNA in a composition is formulated or is to be formulated for intramuscular administration.

In one embodiment, each RNA in a composition is formulated or is to be formulated as particles.

In one embodiment, the particles are lipid nanoparticles (LNP) or lipoplex (LPX) particles.

In one embodiment, the LNP particles comprise ((4-hydroxybutyl)azanediyl)bis(hexane-6,1-diyl)bis(2-hexyldecanoate), 2-[(polyethylene glycol)-2000]-N,N-ditetradecylacetamide, 1,2-Distearoyl-sn-glycero-3-phosphocholine, and cholesterol.

In one embodiment, RNA lipoplex particles are obtainable by mixing RNA with liposomes. In one embodiment, RNA lipoplex particles are obtainable by mixing RNA with lipids.

In one embodiment, RNA is formulated or is to be formulated as colloid. In one embodiment, RNA is formulated or is to be formulated as particles, forming the dispersed phase of a colloid. In one embodiment, 50% or more, 75% or more, or 85% or more of RNA is present in the dispersed phase. In one embodiment, RNA is formulated or is to be formulated as particles comprising RNA and lipids. In one embodiment, particles are formed by exposing RNA, dissolved in an aqueous phase, with lipids, dissolved in an organic phase. In one embodiment, the organic phase comprises ethanol. In one embodiment, particles are formed by exposing RNA, dissolved in an aqueous phase, with lipids, dispersed in an aqueous phase. In one embodiment, the lipids dispersed in an aqueous phase form liposomes.

In one embodiment, each RNA in a composition is mRNA or saRNA.

In one embodiment, the composition or medical preparation is a pharmaceutical composition.

In one embodiment, the composition or medical preparation is a vaccine.

In one embodiment, the pharmaceutical composition further comprises one or more pharmaceutically acceptable carriers, diluents and/or excipients.

In one embodiment, the composition or medical preparation is a kit.

In one embodiment, RNA and optionally particle forming components are in separate vials.

In one embodiment, the kit further comprises instructions for use of the composition or medical preparation for inducing an immune response against coronavirus in a subject.

In one aspect, the present disclosure relates to the composition or medical preparation described herein for pharmaceutical use.

In one embodiment, the pharmaceutical use comprises inducing an immune response against coronavirus in a subject.

In one embodiment, the pharmaceutical use comprises a therapeutic or prophylactic treatment of a coronavirus infection.

In one embodiment, the composition or medical preparation described herein is for administration to a human.

In one embodiment, the coronavirus is a betacoronavirus.

In one embodiment, the coronavirus is a sarbecovirus.

In one embodiment, the coronavirus is SARS-CoV-2.

In one aspect, the present disclosure relates to a method of inducing an immune response against coronavirus and influenza in a subject comprising administering to the subject a composition comprising RNA encoding an amino acid sequence comprising a SARS-CoV-2 S protein, an immunogenic variant thereof, or an immunogenic fragment of the SARS-CoV-2 S protein or the immunogenic variant thereof and an RNA encoding an amino acid sequence comprising an influenza HA protein, an immunogenic variant thereof, or an immunogenic fragment of the influenza HA protein or the immunogenic variant thereof.

In one embodiment, an immunogenic fragment of the SARS-CoV-2 S protein comprises the S1 subunit of the SARS-CoV-2 S protein, or the receptor binding domain (RBD) of the S1 subunit of the SARS-CoV-2 S protein.

Both the N-terminal domain (NTD) and RBD of a coronavirus S protein are known to be sites for binding of antibodies that neutralize virus activity. RBD, in the case of SARS-CoV-2, is the portion of the S protein that angiotensin-converting enzyme 2 (ACE2) on the surface of a host cell. The function of the NTD in the SARS-CoV-2 S protein is not thoroughly understood, but the domain appears to have a role in binding sugar moieties and in facilitating the conformational transition of the S protein from the prefusion to post fusion conformation. Both the NTD and RBD can induce high binding antibody and neutralizing antibody titers.

In some embodiments, the present disclosure provides methods that comprise administering to a human subject a therapeutic dose of a composition comprising an RNA (e.g., an mRNA)) comprising an open reading frame (ORF) that encodes a fusion protein comprising at least two domains of a SARS-CoV-2 Spike (S) protein, and less than the full length spike protein, wherein the RNA is in a lipid nanoparticle.

In one embodiment, the amino acid sequence comprising a SARS-CoV-2 S protein, an immunogenic variant thereof, or an immunogenic fragment of the SARS-CoV-2 S protein or the immunogenic variant thereof is able to form a multimeric complex, in particular a trimeric complex. To this end, an amino acid sequence comprising a SARS-CoV-2 S protein, an immunogenic variant thereof, or an immunogenic fragment of the SARS-CoV-2 S protein or the immunogenic variant thereof may comprise a domain allowing the formation of a multimeric complex, in particular a trimeric complex of the amino acid sequence comprising a SARS-CoV-2 S protein, an immunogenic variant thereof, or an immunogenic fragment of the SARS-CoV-2 S protein or the immunogenic variant thereof. In one embodiment, the domain allowing the formation of a multimeric complex comprises a trimerization domain, for example, a trimerization domain as described herein.

In one embodiment, the amino acid sequence comprising a SARS-CoV-2 S protein, an immunogenic variant thereof, or an immunogenic fragment of the SARS-CoV-2 S protein or the immunogenic variant thereof is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 979 to 1584 of SEQ ID NO: 2, 8 or 9, a nucleotide sequence having at least 99%, 98%, 97%, 96%, 95%, 90%, 85%, or 80% identity to the nucleotide sequence of nucleotides 979 to 1584 of SEQ ID NO: 2, 8 or 9, or a fragment of the nucleotide sequence of nucleotides 979 to 1584 of SEQ ID NO: 2, 8 or 9, or the nucleotide sequence having at least 99%, 98%, 97%, 96%, 95%, 90%, 85%, or 80% identity to the nucleotide sequence of nucleotides 979 to 1584 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 327 to 528 of SEQ ID NO: 1, an amino acid sequence having at least 99%, 98%, 97%, 96%, 95%, 90%, 85%, or 80% identity to the amino acid sequence of amino acids 327 to 528 of SEQ ID NO: 1, or an immunogenic fragment of the amino acid sequence of amino acids 327 to 528 of SEQ ID NO: 1, or the amino acid sequence having at least 99%, 98%, 97%, 96%, 95%, 90%, 85%, or 80% identity to the amino acid sequence of amino acids 327 to 528 of SEQ ID NO: 1.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 111 to 986 of SEQ ID NO: 30, a nucleotide sequence having at least 99%, 98%, 97%, 96%, 95%, 90%, 85%, or 80% identity to the nucleotide sequence of nucleotides 111 to 986 of SEQ ID NO: 30, or a fragment of the nucleotide sequence of nucleotides 111 to 986 of SEQ ID NO: 30, or the nucleotide sequence having at least 99%, 98%, 97%, 96%, 95%, 90%, 85%, or 80% identity to the nucleotide sequence of nucleotides 111 to 986 of SEQ ID NO: 30; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 20 to 311 of SEQ ID NO: 29, an amino acid sequence having at least 99%, 98%, 97%, 96%, 95%, 90%, 85%, or 80% identity to the amino acid sequence of amino acids 20 to 311 of SEQ ID NO: 29, or an immunogenic fragment of the amino acid sequence of amino acids 20 to 311 of SEQ ID NO: 29, or the amino acid sequence having at least 99%, 98%, 97%, 96%, 95%, 90%, 85%, or 80% identity to the amino acid sequence of amino acids 20 to 311 of SEQ ID NO: 29.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 2055 of SEQ ID NO: 2, 8 or 9, a nucleotide sequence having at least 99%, 98%, 97%, 96%, 95%, 90%, 85%, or 80% identity to the nucleotide sequence of nucleotides 49 to 2055 of SEQ ID NO: 2, 8 or 9, or a fragment of the nucleotide sequence of nucleotides 49 to 2055 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2055 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 685 of SEQ ID NO: 1, an amino acid sequence having at least 99%, 98%, 97%, 96%, 95%, 90%, 85%, or 80% identity to the amino acid sequence of amino acids 17 to 685 of SEQ ID NO: 1, or an immunogenic fragment of the amino acid sequence of amino acids 17 to 685 of SEQ ID NO: 1, or the amino acid sequence having at least 99%, 98%, 97%, 96%, 95%, 90%, 85%, or 80% identity to the amino acid sequence of amino acids 17 to 685 of SEQ ID NO: 1.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 49 to 3819 of SEQ ID NO: 2, 8 or 9, a nucleotide sequence having at least 99%, 98%, 97%, 96%, 95%, 90%, 85%, or 80% identity to the nucleotide sequence of nucleotides 49 to 3819 of SEQ ID NO: 2, 8 or 9, or a fragment of the nucleotide sequence of nucleotides 49 to 3819 of SEQ ID NO: 2, 8 or 9, or the nucleotide sequence having at least 99%, 98%, 97%, 96%, 95%, 90%, 85%, or 80% identity to the nucleotide sequence of nucleotides 49 to 3819 of SEQ ID NO: 2, 8 or 9; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 17 to 1273 of SEQ ID NO: 1 or 7, an amino acid sequence having at least 99%, 98%, 97%, 96%, 95%, 90%, 85%, or 80% identity to the amino acid sequence of amino acids 17 to 1273 of SEQ ID NO: 1 or 7, or an immunogenic fragment of the amino acid sequence of amino acids 17 to 1273 of SEQ ID NO: 1 or 7, or the amino acid sequence having at least 99%, 98%, 97%, 96%, 95%, 90%, 85%, or 80% identity to the amino acid sequence of amino acids 17 to 1273 of SEQ ID NO: 1 or 7.

In one embodiment, the amino acid sequence comprising a SARS-CoV-2 S protein, an immunogenic variant thereof, or an immunogenic fragment of the SARS-CoV-2 S protein or the immunogenic variant thereof comprises a secretory signal peptide.

In one embodiment, the secretory signal peptide is fused, preferably N-terminally, to a SARS-CoV-2 S protein, an immunogenic variant thereof, or an immunogenic fragment of the SARS-CoV-2 S protein or the immunogenic variant thereof.

In one embodiment,
(i) the RNA encoding the secretory signal peptide comprises the nucleotide sequence of nucleotides 1 to 48 of SEQ ID NO: 2, 8 or 9, a nucleotide sequence having at least 99%, 98%, 97%, 96%, 95%, 90%, 85%, or 80% identity to the nucleotide sequence of nucleotides 1 to 48 of SEQ ID NO: 2, 8 or 9, or a fragment of the nucleotide sequence of nucleotides 1 to 48 of SEQ ID NO: 2, 8 or 9, or the nucleotide sequence having at least 99%, 98%, 97%, 96%, 95%, 90%, 85%, or 80% identity to the nucleotide sequence of nucleotides 1 to 48 of SEQ ID NO: 2, 8 or 9; and/or
(ii) the secretory signal peptide comprises the amino acid sequence of amino acids 1 to 16 of SEQ ID NO: 1, an amino acid sequence having at least 99%, 98%, 97%, 96%, 95%, 90%, 85%, or 80% identity to the amino acid sequence of amino acids 1 to 16 of SEQ ID NO: 1, or a functional fragment of the amino acid sequence of amino acids 1 to 16 of SEQ ID NO: 1, or the amino acid sequence having at least 99%, 98%, 97%, 96%, 95%, 90%, 85%, or 80% identity to the amino acid sequence of amino acids 1 to 16 of SEQ ID NO: 1.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6, or the nucleotide sequence having at least 99%, 98%, 97%, 96%, 95%, 90%, 85%, or 80% identity to the nucleotide sequence of SEQ ID NO: 6; and/or
(ii) a SARS-CoV-2 S protein, an immunogenic variant thereof, or an immunogenic fragment of the SARS-CoV-2 S protein or the immunogenic variant thereof comprises the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5.

In one embodiment,
(i) the RNA encoding a SARS-CoV-2 S protein, an immunogenic variant thereof, or an immunogenic fragment of the SARS-CoV-2 S protein or the immunogenic variant thereof comprises the nucleotide sequence of nucleotides 54 to 986 of SEQ ID NO: 30, a nucleotide sequence having at least 99%, 98%, 97%, 96%, 95%, 90%, 85%, or 80% identity to the nucleotide sequence of nucleotides 54 to 986 of SEQ ID NO: 30, or a fragment of the nucleotide sequence of nucleotides 54 to 986 of SEQ ID NO: 30, or the nucleotide sequence having at least 99%, 98%, 97%, 96%, 95%, 90%, 85%, or 80% identity to the nucleotide sequence of nucleotides 54 to 986 of SEQ ID NO: 30; and/or
(ii) a SARS-CoV-2 S protein, an immunogenic variant thereof, or an immunogenic fragment of the SARS-CoV-2 S protein or the immunogenic variant thereof comprises the amino acid sequence of amino acids 1 to 311 of SEQ ID NO: 29, an amino acid sequence having at least 99%, 98%, 97%, 96%, 95%, 90%, 85%, or 80% identity to the amino acid sequence of amino acids 1 to 311 of SEQ ID NO: 29, or an immunogenic fragment of the amino acid sequence of amino acids 1 to 311 of SEQ ID NO: 29, or the amino acid sequence having at least 99%, 98%, 97%, 96%, 95%, 90%, 85%, or 80% identity to the amino acid sequence of amino acids 1 to 311 of SEQ ID NO: 29.

In one embodiment, each RNA in a composition is a modified RNA, in particular a stabilized mRNA. In one embodiment, each RNA in a composition comprises a modified nucleoside in place of at least one uridine. In one embodiment, each RNA in a composition comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, each RNA in a composition comprises a modified nucleoside in place of uridine.

In one embodiment, the modified nucleoside is selected from pseudouridine (ψ), N1-methyl-pseudouridine (m1ψ), and 5-methyl-uridine (m5U).

In one embodiment, each RNA in a composition comprises a cap.

In one embodiment, the RNA encoding an amino acid sequence comprising a SARS-CoV-2 S protein, an immunogenic variant thereof, or an immunogenic fragment of the SARS-CoV-2 S protein or the immunogenic variant thereof comprises a 5′ UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In one embodiment, the RNA encoding an amino acid sequence comprising a SARS-CoV-2 S protein, an immunogenic variant thereof, or an immunogenic fragment of the SARS-CoV-2 S protein or the immunogenic variant thereof comprises a 3′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In one embodiment, each RNA in a composition comprises a poly-A sequence.

In one embodiment, the poly-A sequence comprises at least 100 nucleotides.

In one embodiment, the poly-A sequence comprises or consists of the nucleotide sequence of SEQ ID NO: 14.

In one embodiment, each RNA in a composition is formulated as a liquid, a solid, or a combination thereof.

In one embodiment, each RNA in a composition is administered by injection.

In one embodiment, each RNA in a composition is administered by intramuscular administration.

In one embodiment, each RNA in a composition is formulated as particles.

In one embodiment, the particles are lipid nanoparticles (LNP) or lipoplex (LPX) particles.

In one embodiment, the LNP particles comprise ((4-hydroxybutyl)azanediyl)bis(hexane-6,1-diyl)bis(2-hexyldecanoate), 2-[(polyethylene glycol)-2000]-N,N-ditetradecylacetamide, 1,2-Distearoyl-sn-glycero-3-phosphocholine, and cholesterol.

In one embodiment, RNA lipoplex particles are obtainable by mixing RNA with liposomes. In one embodiment, RNA lipoplex particles are obtainable by mixing RNA with lipids.

In one embodiment, RNA is formulated as colloid. In one embodiment, RNA is formulated as particles, forming the dispersed phase of a colloid. In one embodiment, 50% or more, 75% or more, or 85% or more of RNA in a composition are present in the dispersed phase. In one embodiment, RNA is formulated as particles comprising RNA and lipids. In one embodiment, particles are formed by exposing RNA, dissolved in an aqueous phase, with lipids, dissolved in an organic phase. In one embodiment, the organic phase comprises ethanol. In one embodiment, the particles are formed by exposing RNA, dissolved in an aqueous phase, with lipids, dispersed in an aqueous phase. In one embodiment, the lipids dispersed in an aqueous phase form liposomes.

In one embodiment, each RNA in a composition is mRNA or saRNA.

In one embodiment, the method is a method for vaccination against coronavirus.

In one embodiment, the method is a method for therapeutic or prophylactic treatment of a coronavirus infection.

In one embodiment, the subject is a human.

In one embodiment, the coronavirus is a betacoronavirus.

In one embodiment, the coronavirus is a sarbecovirus.

In one embodiment, the coronavirus is SARS-CoV-2.

In one embodiment of the method described herein, the composition is a composition described herein.

In one aspect, the present disclosure relates to a composition or medical preparation described herein for use in a method described herein.

Among other things, the present disclosure teaches that a composition comprising (i) a lipid nanoparticle encapsulated RNA encoding at least a portion (e.g., that is or comprises an epitope) of a SARS-CoV-2-encoded polypeptide (e.g., of a SARS-CoV-2-encoded S protein) and (ii) a lipid nanoparticle encapsulated RNA encoding at least a portion (e.g., that is or comprises an epitope) of an influenza virus-encoded polypeptide (e.g., of an influenza virus-encoded HA protein) can achieve detectable antibody titer against each epitope in serum within 7 days after administration to a population of adult human subjects according to a regimen that includes administration of at least one dose of the vaccine composition. In such compositions, the mRNA encoding at least a portion of a SARS-CoV-2-encoded polypeptide and the mRNA encoding at least a portion of an influenza virus-encoded polypeptide can be formulated in the same, or separate lipid nanoparticle formulations. Moreover, the present disclosure teaches persistence of such antibody titer. In some embodiments, the present disclosure teaches increased such antibody titer when a modified mRNA is used, as compared with that achieved with a corresponding unmodified mRNA.

In some embodiments, a provided regimen includes at least one dose. In some embodiments, a provided regimen includes a first dose and at least one subsequent dose. In some embodiments, the first dose is the same amount as at least one subsequent dose. In some embodiments, the first dose is the same amount as all subsequent doses. In some embodiments, the first dose is a different amount as at least one subsequent dose. In some embodiments, the first dose is a different amount than all subsequent doses. In some embodiments, a provided regimen comprises two doses. In some embodiments, a provided regimen consists of two doses.

In particular embodiments, the immunogenic composition is formulated as a single-dose in a container, e.g., a vial. In some embodiments, the immunogenic composition is formulated as a multi-dose formulation in a vial. In some embodiments, the multi-dose formulation includes at least 2 doses per vial. In some embodiments, the multi-dose formulation includes a total of 2-20 doses per vial, such as, for example, 2, 3, 4, 5, 6, 7, 8, 9, 10, 11, or 12 doses per vial. In some embodiments, each dose in the vial is equal in volume. In some embodiments, a first dose is a different volume than a subsequent dose.

A “stable” multi-dose formulation exhibits no unacceptable levels of microbial growth, and substantially no or no breakdown or degradation of the active biological molecule component(s). As used herein, a “stable” immunogenic composition includes a formulation that remains capable of eliciting a desired immunologic response when administered to a subject.

In some embodiments, the multi-dose formulation remains stable for a specified time with multiple or repeated inoculations/insertions into the multi-dose container. For example, in some embodiments the multi-dose formulation may be stable for at least three days with up to ten usages, when contained within a multi-dose container. In some embodiments, the multi-dose formulations remain stable with 2-20 inoculations/insertions.

In some embodiments, administration of a composition comprising a lipid nanoparticle encapsulated mRNA encoding at least a portion (e.g., that is or comprises an epitope) of a SARS-CoV-2-encoded polypeptide (e.g., of a SARS-CoV-2-encoded S protein), e.g., according to a regimen as described herein, may result in lymphopenia in some subjects (e.g., in all subjects, in most subjects, in about 50% or fewer, in about 40% or fewer, in about 40% or fewer, in about 25% or fewer, in about 20% or fewer, in about 15% or fewer, in about 10% or fewer, in about 5% or fewer, etc). Among other things, the present disclosure teaches that such lymphopenia can resolve over time. For example, in some embodiments, lymphopenia resolves within about 14, about 10, about 9, about 8, about 7 days or less. In some embodiments, lymphopenia is Grade 3, Grade 2, or less.

Thus, among other things, the present disclosure provides compositions comprising a lipid nanoparticle encapsulated mRNA encoding at least a portion (e.g., that is or comprises an epitope) of a SARS-CoV-2-encoded polypeptide (e.g., of a SARS-CoV-2-encoded S protein) and a lipid nanoparticle encapsulated mRNA encoding at least a portion (e.g., that is or comprises an epitope) of an influenza virus-encoded polypeptide (e.g., of an influenza virus-encoded HA protein) that are characterized, when administered to a relevant population of adults, to display certain characteristics (e.g., achieve certain effects) as described herein. In such compositions, the mRNA encoding at least a portion of a SARS-CoV-2-encoded polypeptide and the mRNA encoding at least a portion of an influenza virus-encoded polypeptide can be formulated in the same, or separate lipid nanoparticle formulations. In some embodiments, provided compositions may have been prepared, stored, transported, characterized, and/or used under conditions where temperature does not exceed a particular threshold. Alternatively or additionally, in some embodiments, provided compositions may have been protected from light (e.g., from certain wavelengths) during some or all of their preparation, storage, transport, characterization, and/or use. In some embodiments, one or more features of provided compositions (e.g., mRNA stability, as may be assessed, for example, by one or more of size, presence of particular moiety or modification, etc; lipid nanoparticle stability or aggregation, pH, etc) may be or have been assessed at one or more points during preparation, storage, transport, and/or use prior to administration.

Among other things, the present disclosure documents that certain provided compositions in which nucleotides within an mRNA are not modified (e.g., are naturally occurring A, U, C, G), and/or provided methods relating to such compositions, are characterized (e.g., when administered to a relevant population, which may in some embodiments be or comprise an adult population), by an intrinsic adjuvant effect. In some embodiments, such composition and/or method can induce an antibody and/or a T cell response. In some embodiments, such a composition and/or method can induce a higher T cell response, as compared to conventional vaccines (e.g., non-mRNA vaccines such as protein vaccines).

Alternatively or additionally, the present disclosure documents that provided compositions (e.g., compositions comprising (i) a lipid nanoparticle encapsulated mRNA encoding at least a portion (e.g., that is or comprises an epitope) of a SARS-CoV-2-encoded polypeptide (e.g., of a SARS-CoV-2-encoded S protein) and (ii) a lipid nanoparticle encapsulated mRNA encoding at least a portion (e.g., that is or comprises an epitope) of an influenza virus-encoded polypeptide (e.g., of an influenza-encoded HA protein) in which nucleotides within an mRNA are modified, and/or provided methods relating to such compositions, are characterized (e.g., when administered to a relevant population, which may in some embodiments be or comprise an adult population), by absence of an intrinsic adjuvant effect, or by a reduced intrinsic adjuvant effect as compared with an otherwise comparable composition (or method) with unmodified results. Alternatively or additionally, in some embodiments, such compositions (or methods) are characterized in that they (e.g., when administered to a relevant population, which may in some embodiments be or comprise an adult population) induce an antibody response and/or a CD4+ T cell response. Still further alternatively or additionally, in some embodiments, such compositions (or methods) are characterized in that they (e.g., when administered to a relevant population, which may in some embodiments be or comprise an adult population) induce a higher CD4+ T cell response than that observed with an alternative vaccine format (e.g., a peptide vaccine). In some embodiments involving modified nucleotides, such modified nucleotides may be present, for example, in a 3′ UTR sequence, an antigen-encoding sequence, and/or a 5′UTR sequence. In some embodiments, modified nucleotides are or include one or more modified uracil residues and/or one or more modified cytosine residues.

Among other things, the present disclosure documents that provided compositions (e.g., compositions comprising (i) a lipid nanoparticle encapsulated mRNA encoding at least a portion (e.g., that is or comprises an epitope) of a SARS-CoV-2-encoded polypeptide (e.g., of a SARS-CoV-2-encoded S protein) and (ii) a lipid nanoparticle encapsulated mRNA encoding at least a portion (e.g., that is or comprises an epitope) of an influenza virus-encoded polypeptide (e.g., of an influenza-encoded HA protein)) and/or methods are characterized by (e.g., when administered to a relevant population, which may in some embodiments be or comprise an adult population) sustained expression of an encoded polypeptide (e.g., (i) of a SARS-CoV-2-encoded protein [such as an S protein] or portion thereof, which portion, in some embodiments, may be or comprise an epitope thereof and (ii) of an influenza-encoded protein [such as an HA protein] or portion thereof, which portion, in some embodiments, may be or comprise an epitope thereof). For example, in some embodiments, such compositions and/or methods are characterized in that, when administered to a human, they achieve detectable polypeptide expression in a biological sample (e.g., serum) from such human and, in some embodiments, such expression persists for a period of time that is at least at least 36 hours or longer, including, e.g., at least 48 hours, at least 60 hours, at least 72 hours, at least 96 hours, at least 120 hours, at least 148 hours, or longer.

Those skilled in the art, reading the present disclosure, will appreciate that it describes various compositions comprising one or more mRNA constructs encoding at least a portion (e.g., that is or comprises an epitope) of a SARS-CoV-2-encoded polypeptide (e.g., of a SARS-CoV-2-encoded S protein)) and one or more mRNA constructs encoding at least a portion (e.g., that is or comprises an epitope) of an influenza virus-encoded polypeptide (e.g., of an influenza virus-encoded HA protein)). Such person of ordinary skill, reading the present disclosure, will particularly appreciate that it describes compositions comprising one or more of various mRNA constructs encoding at least a portion of a SARS-CoV-2 S protein, for example at least an RBD portion of a SARS-CoV-2 S protein. Still further, such a person of ordinary skill, reading the present disclosure, will appreciate that it describes particular characteristics and/or advantages of compositions comprising one or more mRNA constructs encoding at least a portion (e.g., that is or comprises an epitope) of a SARS-CoV-2-encoded polypeptide (e.g., of a SARS-CoV-2-encoded S protein) and one or more mRNA constructs encoding at least a portion (e.g., that is or comprises an epitope) of an influenza virus-encoded polypeptide. In some embodiments, a composition may comprise one or more mRNA constructs encoding at least one domain of a SARS-CoV-2 encoded polypeptide (e.g., one or more domains of a SARS-CoV-2 encoded polypeptide as described in WO 2021/159040, including, e.g., an N-terminal domain (NTD) of a SARS-CoV-2 Spike protein, a receptor binding domain (RBD) of a SARS-CoV-2 Spike protein, Heptapeptide repeat sequence 1 (HR1) of a SARS-CoV-2 Spike protein, Heptapeptide repeat sequence 2 (HR1) of a SARS-CoV-2 Spike protein, and/or combinations thereof). Among other things, the present disclosure particularly documents surprising and useful characteristics and/or advantages of compositions comprising one or more RNAs comprising a nucleotide sequence encoding an antigenic polypeptide associated with an influenza virus and certain mRNA constructs encoding a SARS-CoV-2 RBD portion and, in some embodiments, not encoding a full length SARS-CoV-2 S protein. Without wishing to be bound by any particular theory, the present disclosure suggests that RNA that encodes less than a full-length SARS-CoV-2 S protein, and particularly encoding at least an RBD portion of such SARS-CoV-2 S protein may be particularly useful and/or effective for use as or in an immunogenic composition (e.g., a vaccine), and/or for achieving immunological effects as described herein (e.g., generation of SARS-CoV-2 neutralizing antibodies, and/or T cell responses (e.g., CD4+ and/or CD8+ T cell responses)).

In some embodiments, the present disclosure provides a composition comprising an RNA (e.g., mRNA) comprising an open reading frame encoding a polypeptide that comprises a receptor-binding portion of a SARS-CoV-2 S protein, which RNA is suitable for intracellular expression of the polypeptide. In some embodiments, such an encoded polypeptide does not comprise the complete S protein. In some embodiments, the encoded polypeptide comprises the receptor binding domain (RBD), for example, as shown in SEQ ID NO: 5. In some embodiments, the encoded polypeptide comprises the peptide according to SEQ ID NO: 29 or 31. In some embodiments, such an RNA (e.g., mRNA) may be complexed by a (poly) cationic polymer, polyplex(es), protein(s) or peptide(s). In some embodiments, such an RNA may be formulated in a lipid nanoparticle (e.g., ones described herein). In some embodiments, such an RNA (e.g., mRNA) may be particularly useful and/or effective for use as or in an immunogenic composition (e.g., a vaccine), and/or for achieving immunological effects as described herein (e.g., generation of SARS-CoV-2 neutralizing antibodies, and/or T cell responses (e.g., CD4+ and/or CD8+ T cell responses)). In some embodiments, such an RNA (e.g., mRNA) may be useful for vaccinating humans (including, e.g., humans known to have been exposed and/or infected by SARS-CoV-2, and/or humans not known to have been exposed to SARS-CoV-2).

Those skilled in the art, reading the present disclosure, will further appreciate that it describes various mRNA constructs comprising a nucleic acid sequence that encodes a full-length SARS-CoV-2 Spike protein (e.g., including embodiments in which such encoded SARS-CoV-2 Spike protein may comprise at least one or more amino acid substitutions, e.g., proline substitutions as described herein, and/or embodiments in which the mRNA sequence is codon-optimized e.g., for mammalian, e.g., human, subjects). In some embodiments, such a full-length SARS-CoV-2 Spike protein may have an amino acid sequence that is or comprises that set forth in SEQ ID NO: 7. Still further, such a person of ordinary skill, reading the present disclosure, will appreciate, among other things, that it describes particular characteristics and/or advantages of certain mRNA constructs comprising a nucleic acid sequence that encodes a full-length SARS-CoV-2 Spike protein.

Without wishing to be bound by any particular theory, the present disclosure suggests that provided compositions (e.g., compositions comprising one or more mRNA constructs that encode a full-length SARS-CoV-2 S protein and one or more mRNA constructs that encode a an HA protein) may be particularly useful and/or effective for use as or in an immunogenic composition (e.g., a vaccine) in particular subject population (e.g., particular age populations). For example, in some embodiments, such an mRNA composition may be particularly useful in younger (e.g., less than 25 years old, 20 years old, 18 years old, 15 years, 10 years old, or lower) subjects; alternatively or additionally, in some embodiments, such an mRNA composition may be particularly useful in elderly subjects (e.g., over 55 years old, 60 years old, 65 years old, 70 years old, 75 years old, 80 years old, 85 years old, or higher). In particular embodiments, an immunogenic composition comprising such an mRNA construct provided herein exhibits a minimal to modest increase (e.g., no more than 30% increase, no more than 20% increase, or no more than 10% increase, or lower) in dose level and/or dose number-dependent systemic reactogenicity (e.g., fever, fatigue, headache, chills, diarrhea, muscle pain, and/or joint pain, etc.) and/or local tolerability (e.g., pain, redness, and/or swelling, etc.), at least in some subjects (e.g., in some subject age groups); in some embodiments, such reactogenicity and/or local tolerability is observed particularly, in in younger age group (e.g., less than 25 years old, 20 years old, 18 years old or lower) subjects, and/or in older (e.g., elderly) age group (e.g., 65-85 years old). In some embodiments, provided compositions comprising one or more mRNA constructs that encode a full-length SARS-CoV-2 S protein and one or more mRNA constructs that encode an HA protein may be particularly useful and/or effective for use as or in an immunogenic composition (e.g., a vaccine) for inducing SARS-CoV-2 neutralizing antibody and influenza virus neutralizing antibody response levels in a population of subjects that are at high risk for severe diseases associated with SARS-CoV-2 infection and/or influenza virus infection (e.g., an elderly population, for example, 65-85 year-old group).

In some embodiments, methods, compositions, or combinations described herein can be administered to an older adult subject (e.g., a subject 50 years or older, 55 years or older, 60 years and older, or 65 years and older) at increased risk of severe disease caused by RSV infection (e.g., having one of the risk factors described herein). In some embodiments, methods, compositions, or combinations described herein are administered to an older adult (e.g., a subject 60 years and older, or 65 years and older) at increased risk of severe disease caused by RSV infection (e.g., having one of the risk factors described herein).

In some embodiments, methods, compositions, or combinations described herein can be administered to an infant or young child at increased risk of severe disease caused by RSV infection (e.g., having one of the risk factors described herein). In some embodiments, methods, compositions, or combinations described herein are administered to a subject having a condition or that can be exacerbated by RSV infection (e.g., having one of the conditions described herein).

In some embodiments, methods, compositions, or combinations described herein can be administered to a pregnant subject (e.g., a subject at about 32 through about 36 weeks gestational age), e.g., to prevent lower respiratory tract disease (LRTD) and severe LRTD caused by RSV in an infant immediately after birth (e.g., a child from birth through about 6 months of age).

In some embodiments, a higher dose may be administered to elderly subjects (e.g., to subjects 65 year or older) as compared to younger patients. For example, in some embodiments, a dose that is double that given to non-elderly patients (e.g., patients less than 65 years old) is administered to elderly patients (e.g., patients 65 years or older). In some embodiments, elderly patients are administered 60 ug of RNA encoding one or more antigens associated with an infectious agent (e.g., influenza). In some embodiments, elderly patients are administered 60 ug of a tetravalent influenza vaccine (e.g., a vaccine comprising 15 μg of RNA encoding an HA polypeptide associated with an H1N1 influenza A virus, 15 μg of RNA encoding an HA polypeptide associated with an H3N2 influenza A virus, 15 μg of RNA encoding an HA polypeptide associated with a B/Yamagata lineage, and 15 μg of RNA encoding an HA polypeptide associated with a B/Yamagata lineage).

In some embodiments, a person of ordinary skill, reading the present disclosure, will appreciate, among other things, that provided compositions comprising one or more mRNA constructs that encode a full-length SARS-CoV-2 S protein and one or more RNA constructs that encode an HA protein, which exhibit a favorable reactogenicity profile (e.g., as described herein) in younger and elderly age populations, may be particularly useful and/or effective for use as or in an immunogenic composition (e.g., a vaccine) for achieving immunological effects as described herein (e.g., generation of SARS-CoV-2 neutralizing antibodies, influenza virus neutralizing antibodies and/or T cell responses (e.g., CD4+ and/or CD8+ T cell responses)). In some embodiments, the present disclosure also suggests that provided compositions comprising one or more mRNA constructs that encode a full-length SARS-CoV-2 S protein and one or more mRNA constructs that encode an HA protein may be particularly effective to protect against SARS-CoV-2 infection and/or influenza infection, as characterized by earlier clearance of SARS-CoV-2 viral and/or influenza viral RNA in non-human mammalian subjects (e.g., rhesus macaques) that were immunized with immunogenic compositions comprising such mRNA constructs and subsequently challenged by SARS-CoV-2 and/or influenza virus. In some embodiments, such earlier clearance of SARS-CoV-2 viral RNA and/or influenza viral RNA may be observed in the nose of non-human mammalian subjects (e.g., rhesus macaques) that were immunized with immunogenic compositions comprising such mRNA constructs and subsequently challenged by SARS-CoV-2 and/or influenza virus.

In some embodiments, the present disclosure provides a composition comprising one or more RNAs (e.g., one or more mRNAs), each comprising an open reading frame encoding a full-length SARS-CoV-2 S protein (e.g., a full-length SARS-CoV-2 S protein with one or more amino acid substitutions) and one or more RNAs (e.g., one or more mRNAs), each comprising an open reading frame encoding an HA protein, which RNA is suitable for intracellular expression of the polypeptide. In some embodiments, the encoded SARS-CoV-2 protein comprises the amino acid sequence of SEQ ID NO:7, 50, or 69. In some embodiments, the encoded HA protein comprises the amino acid sequence of any one of SEQ ID NOs: 80, 85, 90, 95, 100, or 105. In some embodiments, such an RNA (e.g., mRNA) may be complexed by a (poly) cationic polymer, polyplex(es), protein(s) or peptide(s). In some embodiments, such an RNA may be formulated in a lipid nanoparticle (e.g., ones described herein).

In some embodiments, an immunogenic composition provided herein may comprise a plurality of (e.g., at least two or more, including, e.g., at least three, at least four, at least five, at least six, at least seven, at least eight, at least nine, at least ten, etc.) immunoreactive epitopes of a SARS-CoV-2 polypeptide or variants thereof. In some such embodiments, such a plurality of immunoreactive epitopes may be encoded by a plurality of RNAs (e.g., mRNAs). In some such embodiments, such a plurality of immunoreactive epitopes may be encoded by a single RNA (e.g., mRNA). In some embodiments, nucleic acid sequences encoding a plurality of immunoreactive epitopes may be separated from each other in a single RNA (e.g., mRNA) by a linker (e.g., a peptide linker in some embodiments). Without wishing to be bound by any particular theory, in some embodiments, provided polyepitope immunogenic compositions (including, e.g., those that encode a full-length SARS-CoV-2 spike protein) may be particularly useful, when considering the genetic diversity of SARS-CoV-2 variants, to provide protection against numerous viral variants and/or may offer a greater opportunity for development of a diverse and/or otherwise robust (e.g., persistent, e.g., detectable about 5, 10, 15, 20, 25, 30, 35, 40, 45, 50, 55, 60 or more days after administration of one or more doses) neutralizing antibody and/or T cell response, and in particular a particularly robust T_{H}1-type T cell (e.g., CD4+ and/or CD8+ T cell) response.

In some embodiments, the present disclosure documents that provided compositions and/or methods are characterized by (e.g., when administered to a relevant population, which may in some embodiments be or comprise an adult population) in that they achieve one or more particular therapeutic outcomes (e.g., effective immune responses as described herein and/or detectable expression of an encoded SARS-CoV-2 S protein(s) and an encoded influenza HA protein(s) or immunogenic fragments thereof) with a single administration; in some such embodiments, an outcome may be assessed, for example, as compared to that observed in absence of mRNA vaccines described herein. In some embodiments, an outcome may be assessed as compared to that observed following administration of a monovalent vaccine (e.g., a composition comprising only one of the RNAs disclosed here). In some embodiments, a particular outcome may be achieved at a lower dose than required for one or more alternative strategies.

In some embodiments, the present disclosure provides an immunogenic composition comprising (i) one or more messenger ribonucleic acid (mRNA) polynucleotides, each comprising an open reading frame encoding a polypeptide that comprises a receptor-binding portion of a SARs-CoV-2 S protein, and (ii) one or more messenger ribonucleic acid (mRNA) polynucleotides, each comprising an open reading frame that encodes a polypeptide that comprises an HA protein, wherein the mRNA polynucleotide (i) and the mRNA polynucleotide (ii) are each formulated (together or separately) in at least one lipid nanoparticle. For example, in some embodiments, such a lipid nanoparticle may comprise a molar ratio of 20-60% ionizable cationic lipid, 5-25% non-cationic lipid (e.g., neutral lipid), 25-55% sterol or steroid, and 0.5-15% polymer-conjugated lipid (e.g., PEG-modified lipid). In some embodiments, a sterol or steroid included in a lipid nanoparticle may be or comprise cholesterol. In some embodiments, a neutral lipid may be or comprise 1,2-distearoyl-sn-glycero-3-phosphocholine (DSPC). In some embodiments, a polymer-conjugated lipid may be or comprise PEG2000 DMG. In some embodiments, such an immunogenic composition may comprise a total lipid content of about 1 mg to 10 mg, or 3 mg to 8 mg, or 4 mg to 6 mg. In some embodiments, such an immunogenic composition may comprise a total lipid content of about 5 mg/mL-15 mg/mL or 7.5 mg/mL-12.5 mg/mL or 9-11 mg/mL. In some embodiments, such a composition is provided in an effective amount to induce an immune response in a subject administered at least one dose of the immunogenic composition. In some embodiments, a polypeptide encoded by the mRNA polynucleotide (i) does not comprise the complete S protein. In some embodiments, in the mRNA polynucleotides in such an immunogenic composition are not self-replicating RNA.

In some embodiments, a composition disclosed herein can induce an immune response against a first infectious agent and a second infectious agent. In some embodiments, a composition can induce an immune response against a coronavirus an another respiratory disease. In some embodiments, a composition can induce an immune response against SARS-CoV-2 and an influenza virus.

In some embodiments, an immune response may comprise generation of a binding antibody titer against SARS-CoV-2 protein (including, e.g., a stabilized prefusion spike trimer in some embodiments) and/or an influenza virus protein, or fragments thereof. In some embodiments, an immune response may comprise generation of a binding antibody titer against the receptor binding domain (RBD) of the SARS-CoV-2 spike protein. In some embodiments, a provided immunogenic composition has been established to achieve a detectable binding antibody titer after administration of a first dose, with seroconversion in at least 70% (including, e.g., at least 80%, at least 90%, at least 95% and up to 100%) of a population of subjects receiving such a provided immunogenic composition, for example, by about 2 weeks.

In some embodiments, an immune response may comprise generation of a neutralizing antibody titer against SARS-CoV-2 protein (including, e.g., a stabilized prefusion spike trimer in some embodiments) and/or an influenza virus protein, or fragments thereof. In some embodiments, an immune response may comprise generation of a neutralizing antibody titer against the receptor binding domain (RBD) of the SARS-CoV-2 spike protein. In some embodiments, a provided immunogenic composition has been established to achieve a neutralizing antibody titer in an appropriate system (e.g., in a human infected with SARS-CoV-2, influenza virus and/or populations thereof, and/or in model systems therefor). For example, in some embodiments, such neutralizing antibody titer may have been demonstrated in one or more of a population of humans, a non-human primate model (e.g., rhesus macaques), and/or a mouse model.

In some embodiments, a neutralizing antibody titer is a titer that is (e.g., that has been established to be) sufficient to reduce viral infection of B cells relative to that observed for an appropriate control (e.g., an unvaccinated control subject, or a subject vaccinated with a live attenuated viral vaccine, an inactivated viral vaccine, or a protein subunit viral vaccine, or a combination thereof). In some such embodiments, such reduction is of at least 30%, 35%, 40%, 45%, 50%, 55%, 60%, 65%, 70%, 75%, 80%, 85%, 90%, 95%, or more.

In some embodiments, a neutralizing antibody titer is a titer that is (e.g., that has been established to be) sufficient to reduce the rate of asymptomatic viral infection relative to that observed for an appropriate control (e.g., an unvaccinated control subject, or a subject vaccinated with a live attenuated viral vaccine, an inactivated viral vaccine, or a protein subunit viral vaccine, or a combination thereof). In some such embodiments, such reduction is of at least 30%, 35%, 40%, 45%, 50%, 55%, 60%, 65%, 70%, 75%, 80%, 85%, 90%, 95%, or more. In some embodiments, such reduction can be characterized by assessment of SARS-CoV-2 N protein and/or influenza virus serology. Significant protection against asymptomatic infection can also be confirmed by real life observations (see e.g., the SARS-CoV-2 related results summarized in Dagan N. et al., N Engl J Med. 2021, doi: 10.1056/NEJMoa2101765. Epub ahead of print. PMID: 33626250)

In some embodiments, a neutralizing antibody titer is a titer that is (e.g., that has been established to be) sufficient to reduce or block fusion of virus with epithelial cells and/or B cells of a vaccinated subject relative to that observed for an appropriate control (e.g., an unvaccinated control subject, or a subject vaccinated with a live attenuated viral vaccine, an inactivated viral vaccine, or a protein subunit viral vaccine, or a combination thereof). In some such embodiments, such reduction is of at least 30%, 35%, 40%, 45%, 50%, 55%, 60%, 65%, 70%, 75%, 80%, 85%, 90%, 95%, or more.

In some embodiments, induction of a neutralizing antibody titer may be characterized by an elevation in the number of B cells, which in some embodiments may include plasma cells, class-switched IgG1- and IgG2-positive B cells, and/or germinal center B cells. In some embodiments, a provided immunogenic composition has been established to achieve such an elevation in the number of B cells in an appropriate system (e.g., in a human infected with SARS-CoV-2 and/or a population thereof, and/or in a model system therefor). For example, in some embodiments, such an elevation in the number of B cells may have been demonstrated in one or more of a population of humans, a non-human primate model (e.g., rhesus macaques), and/or a mouse model. In some embodiments, such an elevation in the number of B cells may have been demonstrated in draining lymph nodes and/or spleen of a mouse model after (e.g., at least 7 days, at least 8 days, at least 9 days, at least 10 days, at least 11 days, at least 12 days, at least 13 days, at least 14 days, after) immunization of such a mouse model with a provided immunogenic composition.

In some embodiments, induction of a neutralizing antibody titer may be characterized by a reduction in the number of circulating B cells in blood. In some embodiments, a provided immunogenic composition has been established to achieve such a reduction in the number of circulating B cells in blood of an appropriate system (e.g., in a human infected with SARS-CoV-2 and/or a population thereof, and/or in a model system therefor). For example, in some embodiments, such a reduction in the number of circulating B cells in blood may have been demonstrated in one or more of a population of humans, a non-human primate model (e.g., rhesus macaques), and/or a mouse model. In some embodiments, such a reduction in the number of circulating B cells in blood may have been demonstrated in a mouse model after (e.g., at least 4 days, at least 5 days, at least 6 days, at least 7 days, at least 8 days, at least 9 days, at least 10 days, after) immunization of such a mouse model with a provided immunogenic composition. Without wishing to be bound by theory, a reduction in circulating B cells in blood may be due to B cell homing to lymphoid compartments.

In some embodiments, an immune response induced by a provided immunogenic composition may comprise an elevation in the number of T cells. In some embodiments, such an elevation in the number of T cells may include an elevation in the number of T follicular helper (T_{FH}) cells, which in some embodiments may comprise one or more subsets with ICOS upregulation. One of skilled in the art will understand that proliferation of TH in germinal centres is integral for generation of an adaptive B-cell response, and also that in humans, T_{FH} occurring in the circulation after vaccination is typically correlated with a high frequency of antigen-specific antibodies. In some embodiments, a provided immunogenic composition has been established to achieve such an elevation in the number of T cells (e.g., T_{FH} cells) in an appropriate system (e.g., in a human infected with SARS-CoV-2 and/or a population thereof, and/or in a model system therefor). For example, in some embodiments, such an elevation in the number of T cells (e.g., T_{FH} cells) may have been demonstrated in one or more of a population of humans, a non-human primate model (e.g., rhesus macaques), and/or a mouse model. In some embodiments, such an elevation in the number of T cells (e.g., e.g., T_{FH} cells) may have been demonstrated in draining lymph nodes, spleen, and/or blood of a mouse model after (e.g., at least 4 days, at least 5 days, at least 6 days, at least 7 days, at least 8 days, at least 9 days, at least 10 days, at least 11 days, at least 12 days, at least 13 days, at least 14 days, after) immunization of such a mouse model with a provided immunogenic composition.

In some embodiments, a protective response against SARS-CoV-2 and/or influenza virus induced by a provided immunogenic composition has been established in an appropriate model system for SARS-CoV-2 and/or influenza. For example, in some embodiments, such a protective response may have been demonstrated in an animal model, e.g., a non-human primate model (e.g., rhesus macaques) and/or a mouse model. In some embodiments, a non-human primate (e.g., rhesus macaque) or a population thereof that has/have received at least one immunization with a provided immunogenic composition is/are challenged with SARS-CoV-2 and/or influenza virus, e.g., through intranasal and/or intratracheal route. In some embodiments, such a challenge may be performed several weeks (e.g., 5-10 weeks) after at least one immunization (including, e.g., at least two immunizations) with a provided immunogenic composition. In some embodiments, such a challenge may be performed when a detectable level of a SARS-CoV-2 neutralizing titer and/or an influenza neutralizing titer (e.g., antibody response to SARS-CoV-2 spike protein, influenza virus HA protein and/or fragments thereof, including, e.g., but not limited to a stabilized prefusion spike trimer, S-2P, RBD, and/or HA protein) is achieved in non-human primate(s) (e.g., rhesus macaque(s)) that has received at least one immunization (including, e.g., at least two immunizations) with a provided immunogenic composition. In some embodiments, a protective response is characterized by absence of or reduction in detectable viral RNA in bronchoalveolar lavage (BAL) and/or nasal swabs of challenged non-human primate(s) (e.g., rhesus macaque(s)). In some embodiments, immunogenic compositions described herein may have been characterized in that a larger percent of challenged animals, for example, non-human primates in a population (e.g., rhesus macaques), that have received at least one immunization (including, e.g., at least two immunizations) with a provided immunogenic composition display absence of detectable RNA in their BAL and/or nasal swab, as compared to a population of non-immunized animals, for example, non-human primates (e.g., rhesus macaques). In some embodiments, immunogenic compositions described herein may have been characterized in that challenged animals, for example, non-human in a population (e.g., rhesus macaques), that have received at least one immunization (including, e.g., at least two immunizations) with a provided immunogenic composition may show clearance of viral RNA in nasal swab no later than 10 days, including, e.g., no later than 8 days, no later than 6 days, no later than 4 days, etc., as compared to a population of non-immunized animals, for example, non-human primates (e.g., rhesus macaques).

In some embodiments, immunogenic compositions described herein when administered to subjects in need thereof do not substantially increase the risk of vaccine-associated enhanced respiratory disease. In some embodiments, such vaccine-associated enhanced respiratory disease may be associated with antibody-dependent enhancement of replication and/or with vaccine antigens that induced antibodies with poor neutralizing activity and Th2-biased responses. In some embodiments, immunogenic compositions described herein when administered to subjects in need thereof do not substantially increase the risk of antibody-dependent enhancement of replication.

In some embodiments, a single dose of an mRNA composition (e.g., formulated in lipid nanoparticles) can induce a therapeutic antibody response in less than 10 days of vaccination. In some embodiments, such a therapeutic antibody response may be characterized in that when such an mRNA vaccine can induce production of about 10-100 ug/mL IgG measured at 10 days after vaccination at a dose of 0.1 to 10 ug or 0.2-5 ug in an animal model. In some embodiments, such a therapeutic antibody response may be characterized in that such an mRNA vaccine induces about 100-1000 ug/mL IgG measured at 20 days of vaccination at a dose of 0.1 to 10 ug or 0.2-5 ug in an animal model. In some embodiments, a single dose may induce a pseudovirus-neutralization titer, as measured in an animal model, of 10-200 pVN50 titer 15 days after vaccination. In some embodiments, a single dose may induce a pseudovirus-neutralization titer, as measured in an animal model, of 50-500 pVN50 titer 15 days after vaccination.

In some embodiments, a single dose of an mRNA composition can expand antigen-specific CD8 and/or CD4 T cell response by at least at 50% or more (including, e.g., at least 60%, at least 70%, at least 80%, at least 90%, at least 95%, or more), as compared to that observed in absence of such an mRNA composition. In some embodiments, a single dose of an mRNA composition can expand antigen-specific CD8 and/or CD4 T cell response by at least at 1.5-fold or more (including, e.g., at least 2-fold, at least 3-fold, at least 5-fold, at least 10-fold, at least 50-fold, at least 100-fold, at least 500-fold, at least 1000-fold, or more), as compared to that observed in absence of such an mRNA composition.

In some embodiments, a regimen (e.g., a single dose of an mRNA composition) can expand T cells that exhibit a Th1 phenotype (e.g., as characterized by expression of IFN-gamma, IL-2, IL-4, and/or IL-5) by at least at 50% or more (including, e.g., at least 60%, at least 70%, at least 80%, at least 90%, at least 95%, or more), as compared to that observed in absence of such a regimen. In some embodiments, a regimen (e.g., a single dose of an mRNA composition) can expand T cells that exhibit a Th1 phenotype (e.g., as characterized by expression of IFN-gamma, IL-2, IL-4, and/or IL-5), for example by at least at 1.5-fold or more (including, e.g., at least 2-fold, at least 3-fold, at least 5-fold, at least 10-fold, at least 50-fold, at least 100-fold, at least 500-fold, at least 1000-fold, or more), as compared to that observed in absence of such a regimen. In some embodiments, a T-cell phenotype may be or comprise a Th1-dominant cytokine profile (e.g., as characterized by INF-gamma positive and/or IL-2 positive), and/or no by or biologically insignificant IL-4 secretion.

In some embodiments, a regimen as described herein (e.g., one or more doses of an mRNA composition) induces and/or achieves production of RBD-specific CD4+ T cells. Among other things, the present disclosure documents that mRNA compositions encoding an RBD-containing portion of a SARS-CoV-2 spike protein (e.g., a full-length SARS-CoV-2 spike protein) and one or more HA proteins may be particularly useful and/or effective in such induction and/or production of RBD-specific CD4+ T cells. In some embodiments, RBD-specific CD4+ T-cells induced by an mRNA composition described herein (e.g., by an mRNA composition that encodes an RBD-containing-portion of a SARS-CoV-2 spike protein and an HA protein demonstrate a Th1-dominant cytokine profile (e.g., as characterized by INF-gamma positive and/or IL-2 positive), and/or by no or biologically insignificant IL-4 secretion.

In some embodiments, characterization of CD4+ and/or CD8+ T cell responses (e.g., described herein) in subjects receiving mRNA compositions (e.g., as described herein) may be performed using ex vivo assays using PBMCs collected from the subjects.

In some embodiments, immunogenicity of mRNA compositions described herein may be assessed by one of or more of the following serological immunogenicity assays: detection of IgG, IgM, and/or IgA to SARS-CoV-2 S protein and/or HA protein present in blood samples of a subject receiving a provided mRNA composition, and/or neutralization assays using SARS-CoV-2 pseudovirus influenza pseudovirus and/or a wild-type SARS-CoV-2 virus or a wild-type influenza virus.

In some embodiments, an mRNA composition (e.g., as described herein) provide a relatively low adverse effect (e.g., Grade 1-Grade 2 pain, redness and/or swelling) within 7 days after vaccinations at a dose of 10 ug-100 ug or 1 ug-50 ug. In some embodiments, mRNA compositions (e.g., as described herein) provide a relatively low observation of systemic events (e.g., Grade 1-Grade 2 fever, fatigue, headache, chills, vomiting, diarrhea, muscle pain, joint pain, medication, and combinations thereof) within 7 days after vaccinations at a dose of 10 ug-100 ug.

In some embodiments, mRNA compositions are characterized in that when administered to subjects at 10-100 ug dose or 1 ug-50 ug, IgG directed to a SARS-CoV-2 immunogenic protein, an influenza virus immunogenic protein, and/or fragments thereof (e.g., spike protein receptor binding domain, and/or HA protein) may be produced at a level of 100-100,000 U/mL or 500-50,000 U/mL 21 days after vaccination.

In some embodiments, an mRNA encodes a natively-folded trimeric receptor binding protein of SARS-CoV-2. In some embodiments, an mRNA encodes a variant of such receptor binding protein such that the encoded variant binds to ACE2 at a Kd of 10 pM or lower, including, e.g., at a Kd of 9 pM, 8 pM, 7 pM, 6 pM, 5 pM, 4 pM, or lower. In some embodiments, an mRNA encodes a variant of such receptor binding protein such that the encoded variant binds to ACE2 at a Kd of 5 pM. In some embodiments, an mRNA encodes a trimeric receptor binding portion of SARS-CoV-2 that comprises an ACE2 receptor binding site. In some embodiments, an mRNA comprises a coding sequence for a receptor-binding portion of SARS-CoV-2 and a trimerization domain (e.g., a natural trimerization domain (foldon) of T4 fibritin) such that the coding sequence directs expression of a trimeric protein that has an ACE2 receptor binding site and binds ACE2. In some embodiments, an mRNA encodes a trimeric receptor binding portion of SARS-CoV-2 or a variant thereof such that its Kd is smaller than that for a monomeric receptor-binding domain (RBD) of SARS-CoV-2. For example, in some embodiments, an mRNA encodes a trimeric receptor binding portion of SARS-CoV-2 or a variant thereof such that its Kd is at least 10-fold (including, e.g., at least 50-fold, at least 100-fold, at least 500-fold, at least 1000-fold, etc.) smaller than that for a RBD of SARS-CoV-2.

In some embodiments, a trimer receptor binding portion of SARS-CoV-2 encoded by an mRNA (e.g., as described herein) may be determined to have a size of about 3-4 angstroms when it is complexed with ACE2 and B⁰AT1 neutral amino acid transporter in a closed conformation, as characterized by electron cryomicroscopy (cryoEM). In some embodiments, geometric mean SARS-CoV-2 neutralizing titer that characterizes and/or is achieved by an mRNA composition or method as described herein can reach at least 1.5-fold, including, at least 2-fold, at least 2.5-fold, at least 3-fold, or higher, that of a COVID-19 convalescent human panel (e.g., a panel of sera from COVID-19 convalescing humans obtained 20-40 days after the onset of symptoms and at least 14 days after the start of asymptomatic convalescence.

In some embodiments, mRNA compositions as provided herein may be characterized in that subjects who have been treated with such compositions (e.g., with at least one dose, at least two doses, etc) may show reduced and/or more transient presence of viral RNA in relevant site(s) (e.g., nose and/or lungs, etc, and/or any other tissue susceptible to infection) as compared with an appropriate control (e.g., an established expected level for a comparable subject or population not having been so treated and having been exposed to virus under reasonably comparable exposure conditions) In some embodiments, the RBD antigen expressed by an mRNA construct (e.g., as described herein) can be modified by addition of a T4-fibritin-derived “foldon” trimerization domain, for example, to increase its immunogenicity.

In some embodiments, mRNA compositions and/or methods described herein are characterized in that certain local reactions (e.g., pain, redness, and/or swelling, etc.) and/or systemic events (e.g., fever, fatigue, headache, etc.) may appear and/or peak at Day 2 after vaccination. In some embodiments, mRNA compositions described herein are characterized in that certain local reactions (e.g., pain, redness, and/or swelling, etc.) and/or systemic events (e.g., fever, fatigue, headache, etc.) may resolve by Day 7 after vaccination.

In some embodiments, mRNA compositions and/or methods described herein are characterized in that no Grade 1 or greater change in routine clinical laboratory values or laboratory abnormalities are observed in subjects receiving mRNA compositions (e.g., as described herein). Examples of such clinical laboratory assays may include lymphocyte count, hematological changes, etc.

In some embodiments, mRNA compositions and/or methods described herein are characterized in that by 21 days after a first dose (e.g., 10-100 μg inclusive or 1 μg-50 μg inclusive), geometric mean concentrations (GMCs) of IgG directed to a SARS-CoV-2 S polypeptide, influenza virus HA protein, or immunogenic fragments thereof (e.g., RBD) may reach 200-3000 units/mL or 500-3000 units/mL or 500-2000 units/mL, compared to 602 units/mL for a panel of COVID-19 convalescent human sera. In some embodiments, mRNA compositions described herein are characterized in that by 7 days after a second dose (e.g., 10-30 ug inclusive; or 1 ug-50 ug inclusive), geometric mean concentrations (GMCs) of IgG directed to a SARS-CoV-2 spike polypeptide, HA polypeptide, or immunogenic fragments thereof (e.g., RBD) may increase by at least 8-fold or higher, including, e.g., at least 9-fold, at least 10-fold, at least 15-fold, at least 20-fold, at least 25-fold, at least 30-fold, at least 35-fold, at least 40-fold, or higher. In some embodiments, mRNA compositions described herein are characterized in that by 7 days after a second dose (e.g., 10-30 μg inclusive; or 1 μg-50 μg inclusive), geometric mean concentrations (GMCs) of IgG directed to a SARS-CoV-2 S polypeptide, influenza HA polypeptide, or immunogenic fragments thereof (e.g., RBD) may increase to 1500 units/mL to 40,000 units/mL or 4000 units/mL to 40,000 units/mL. In some embodiments, antibody concentrations described herein can persist to at least 20 days or longer, including, e.g., at least 25 days, at least 30 days, at least 35 days, at least 40 days, at least 45 days, at least 50 days, after a first dose, or at least 10 days or longer, including, e.g., at least 15 days, at least 20 days, at least 25 days, or longer, after a second dose. In some embodiments, antibody concentrations can persist to 35 days after a first dose, or at least 14 days after a second dose.

In some embodiments, mRNA compositions described herein are characterized in that when measured at 7 days after a second dose (e.g., 1-50 μg inclusive), GMC of IgG directed to a SARS-CoV-2 S polypeptide, an influenza virus HA polypeptide, or immunogenic fragments thereof (e.g., RBD) is at least 30% higher (including, e.g., at least 40% higher, at least 50% higher, at least 60%, higher, at least 70% higher, at least 80% higher, at least 90% higher, at least 95% higher, as compared to antibody concentrations observed in a panel of COVID-19 convalescent human serum or influenza convalescent human serum. In many embodiments, geometric mean concentration (GMC) of IgG described herein is GMCs of RBD-binding IgG.

In some embodiments, mRNA compositions described herein are characterized in that when measured at 7 days after a second dose (e.g., 10-50 μg inclusive), GMC of IgG directed to a SARS-CoV-2 S polypeptide, an influenza virus HA polypeptide, or immunogenic fragments thereof (e.g., RBD) is at least 1.1-fold higher (including, e.g., at least 1.5-fold, at least 2-fold, at least 3-fold, at least 4-fold, at least 5-fold, at least 6-fold higher, at least 7-fold higher, at least 8-fold higher, at least 9-fold higher, at least 10-fold higher, at least 15-fold higher, at least 20-fold higher, at least 25-fold higher, at least 30-fold higher), as compared to antibody concentrations observed in a panel of COVID-19 convalescent human serum and/or a panel of influenza convalescent human serum, In many embodiments, geometric mean concentration (GMC) of IgG described herein is GMCs of RBD-binding IgG.

In some embodiments, mRNA compositions described herein are characterized in that when measured at 21 days after a second dose, GMC of IgG directed to a SARS-CoV-2 S polypeptide, an influenza virus HA polypeptide or immunogenic fragments thereof (e.g., RBD) is at least 5-fold higher (including, e.g., at least 6-fold higher, at least 7-fold higher, at least 8-fold higher, at least 9-fold higher, at least 10-fold higher, at least 15-fold higher, at least 20-fold higher, at least 25-fold higher, at least 30-fold higher), as compared to antibody concentrations observed in a panel of COVID-19 convalescent or influenza convalescent human serum, In many embodiments, geometric mean concentration (GMC) of IgG described herein is GMCs of RBD-binding IgG.

In some embodiments, mRNA compositions and/or methods described herein are characterized in that an increase (e.g., at least 30%, at least 40%, at least 50%, or more) in SARS-CoV-2 and/or influenza neutralizing geometric mean titers (GMTs) is observed 21 days after a first dose. In some embodiments, mRNA compositions described herein are characterized in that a substantially greater serum neutralizing GMTs are achieved 7 days after subjects receive a second dose (e.g., 10 μg-30 μg inclusive), reaching 150-300, compared to 94 for a COVID-19 convalescent serum panel.

In some embodiments, mRNA compositions and/or methods described herein are characterized in that 7 days after administration of the second dose, the protective efficacy is at least 60%, e.g., at least 70%, at least 80%, at least 90, or at least 95%. In one embodiment, mRNA compositions and/or methods described herein are characterized in that 7 days after administration of the second dose, the protective efficacy is at least 70%. In one embodiment, mRNA compositions and/or methods described herein are characterized in that 7 days after administration of the second dose, the protective efficacy is at least 80%. In one embodiment, mRNA compositions and/or methods described herein are characterized in that 7 days after administration of the second dose, the protective efficacy is at least 90%. In one embodiment, mRNA compositions and/or methods described herein are characterized in that 7 days after administration of the second dose, the protective efficacy is at least 95%.

In some embodiments, an RNA composition provided herein is characterized in that it induces an immune response against SARS-CoV-2 and or influenza virus after at least 7 days after a dose (e.g., after a second dose). In some embodiments, an RNA composition provided herein is characterized in that it induces an immune response against SARS-CoV-2 and/or an influenza virus in less than 14 days after a dose (e.g., after a second dose). In some embodiments, an RNA composition provided herein is characterized in that it induces an immune response against SARS-CoV-2 and/or an influenza virus after at least 7 days after a vaccination regimen. In some embodiments, a vaccination regimen comprises a first dose and a second dose. In some embodiments, a first dose and a second dose are administered by at least 21 days apart. In some such embodiments, an immune response against SARS-CoV-2 and/or an influenza virus is induced at least after 28 days after a first dose.

In some embodiments, mRNA compositions and/or methods described herein are characterized in that geometric mean concentration (GMCs) of antibodies directed to a SARS-CoV-2 spike polypeptide, an influenza virus HA polypeptide or immunogenic fragments thereof (e.g., RBD), as measured in serum from subjects receiving mRNA compositions of the present disclosure (e.g., at a dose of 10-30 μg inclusive), is substantially higher than in a convalescent serum panel (e.g., as described herein). In some embodiments where a subject may receive a second dose (e.g., 21 days after 1 first dose), geometric mean concentration (GMCs) of antibodies directed to a SARS-CoV-2 spike polypeptide, an influenza virus HA polypeptide, or immunogenic fragments thereof (e.g., RBD), as measured in serum from the subject, may be 8.0-fold to 50-fold higher than a convalescent serum panel GMC. In some embodiments where a subject may receive a second dose (e.g., 21 days after 1 first dose), geometric mean concentration (GMCs) of antibodies directed to a SARS-CoV-2 spike polypeptide, an influenza virus HA polypeptide or immunogenic fragments thereof (e.g., RBD), as measured in serum from the subject, may be at least 8.0-fold or higher, including, e.g., at least 10-fold, at least 20-fold, at least 30-fold, at least 40-fold, at least 50-fold, at least 60-fold or higher, as compared to a convalescent serum panel GMC.

In some embodiments, mRNA compositions and/or methods described herein are characterized in that the SARS-CoV-2 neutralizing geometric mean titer and/or influenza virus neutralizing geometric mean titer, as measured at 28 days after a first dose or 7 days after a second dose, may be at least 1.5-fold or higher (including, e.g., at least 2-fold, at least 2.5-fold, at least 3-fold, at least 3.5-fold or higher), as compared to a neutralizing GMT of a convalescent serum panel.

In some embodiments, a regimen administered to a subject may be or comprise a single dose. In some embodiments, a regimen administered to a subject may comprise a plurality of doses (e.g., at least two doses, at least three doses, or more). In some embodiments, a regimen administered to a subject may comprise a first dose and a second dose, which are given at least 2 weeks apart, at least 3 weeks apart, at least 4 weeks apart, or more. In some embodiments, such doses may be at least 1 month, at least 2 months, at least 3 months, at least 4 months, at least 5 months, at least 6 months, at least 7 months, at least 8 months, at least 9 months, at least 10 months, at least 11 months, at least 12 months, or more apart. In some embodiments, doses may be administered days apart, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days apart. In some embodiments, doses may be administered about 1 to about 3 weeks apart, or about 1 to about 4 weeks apart, or about 1 to about 5 weeks apart, or about 1 to about 6 weeks apart, or about 1 to more than 6 weeks apart. In some embodiments, doses may be separated by a period of about 7 to about 60 days, such as for example about 14 to about 48 days, etc. In some embodiments, a minimum number of days between doses may be about 1, 2, 3, 4, 5, 6, 7, 8, 9, 10, 11, 12, 13, 14, 15, 16, 17, 18, 19, 20, 21 or more. In some embodiments, a maximum number of days between doses may be about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or fewer. In some embodiments, doses may be about 21 to about 28 days apart. In some embodiments, doses may be about 19 to about 42 days apart. In some embodiments, doses may be about 7 to about 28 days apart. In some embodiments, doses may be about 14 to about 24 days. In some embodiments, doses may be about 21 to about 42 days.

In some embodiments, particularly for compositions established to achieve elevated antibody and/or T-cell titres for a period of time longer than about 3 weeks—e.g., in some embodiments, a provided composition is established to achieve elevated antibody and/or T-cell titres (e.g., specific for a relevant portion of a SARS-CoV-2 spike protein or an influenza virus HA protein) for a period of time longer than about 3 weeks; in some such embodiments, a dosing regimen may involve only a single dose, or may involve two or more doses, which may, in some embodiments, be separated from one another by a period of time that is longer than about 21 days or three weeks. For example, in some such embodiments, such period of time may be about 4 weeks, 5 weeks, 6 weeks 7 weeks, 8 weeks, 9 weeks, 10 weeks, 11 weeks, 12 weeks, 13 weeks, 14 weeks, 15 wees, 16 weeks, 17 weeks, 18 weeks, 19 weeks, 20 weeks or more, or about 1 month, 2 months, 3 months, 4 months, 5 months, 6 months, 7 months, 8 months, 9 months, 10, months, 11 months, 12 months or more, or in some embodiments about a year or more.

In some embodiments, a first dose and a second dose (and/or other subsequent dose) may be administered by intramuscular injection. In some embodiments, a first dose and a second dose may be administered in the deltoid muscle. In some embodiments, a first dose and a second dose may be administered in the same arm. In some embodiments, an mRNA composition described herein is administered (e.g., by intramuscular injection) as a series of two doses (e.g., 0.3 mL each) 21 days part. In some embodiments, each dose is about 30 ug. In some embodiments, each dose may be higher than 30 ug, e.g., about 40 ug, about 50 ug, about 60 ug. In some embodiments, each dose may be lower than 30 ug, e.g., about 20 ug, about 10 ug, about 5 ug, etc. In some embodiments, each dose is about 3 ug or lower, e.g., about 1 ug. In some such embodiments, an mRNA composition described herein is administered to subjects of age 16 or older (including, e.g., 16-85 years). In some such embodiments, an mRNA composition described herein is administered to subjects of age 18-55. In some such embodiments, an mRNA composition escribed herein is administered to subjects of age 56-85. In some embodiments, an mRNA composition described herein is administered (e.g., by intramuscular injection) as a single dose.

In some embodiments, mRNA compositions and/or methods described herein are characterized in that RBD-specific IgG (e.g., polyclonal response) induced by such mRNA compositions and/or methods exhibit a higher binding affinity to RBD, as compared to a reference human monoclonal antibody with SARS-CoV-2 RBD-binding affinity (e.g., CR3022 as described in J. ter Meulen et al., PLOS Med. 3, e237 (2006).) In some embodiments, mRNA compositions and/or methods described herein are characterized in that HA-specific IgG (e.g., polyclonal response) induced by such mRNA compositions and/or methods exhibit a higher binding affinity to HA, as compared to a reference human monoclonal antibody with HA binding affinity.

In some embodiments, mRNA compositions and/or methods described herein are characterized in that sera of vaccinated subjects display neutralizing activity across a panel (e.g., at least 10, at least 15, or more) of SARS-CoV-2 spike variants and/or influenza virus HA variants. In some embodiments, such SARs-CoV-2 spike variants include mutations in RBD (e.g., but not limited to Q321L, V341I, A348T, N354D, S359N, V367F, K378R, R408I, Q409E, A435S, N439K, K458R, I472V, G476S, S477N, V483A, Y508H, H519P, etc., as compared to SEQ ID NO: 1), and/or mutations in spike protein (e.g., but not limited to D614G, etc., as compared to SEQ ID NO: 1). Those skilled in the art are aware of various spike variants, and/or resources that document them (e.g., the Table of mutating sites in Spike maintained by the COVID-19 Viral Genome Analysis Pipeline and found at https_//cov.lanl.gov/components/sequence/COV/int_sites_tbls.comp) (last accessed 24 Aug. 2020), and, reading the present specification, will appreciate that mRNA compositions and/or methods described herein can be characterized for their ability to induce sera in vaccinated subject that display neutralizing activity with respect to any or all of such variants and/or combinations thereof.

In particular embodiments, mRNA compositions encoding RBD of a SARS-CoV-2 spike protein are characterized in that sera of vaccinated subjects display neutralizing activity across a panel (e.g., at least 10, at least 15, or more) of SARs-CoV-2 spike variants including RBD variants (e.g., but not limited to Q321L, V341I, A348T, N354D, S359N, V367F, K378R, R408I, Q409E, A435S, N439K, K458R, I472V, G476S, S477N, V483A, Y508H, H519P, etc., as compared to SEQ ID NO: 1) and spike protein variants (e.g., but not limited to D614G, as compared to SEQ ID NO: 1).

In particular embodiments, mRNA compositions encoding a SARS-CoV-2 spike protein variant that includes two consecutive proline substitutions at amino acid positions 986 and 987, at the top of the central helix in the S2 subunit, are characterized in that sera of vaccinated subjects display neutralizing activity across a panel (e.g., at least 10, at least 15, or more) of SARs-CoV-2 spike variants including RBD variants (e.g., but not limited to Q321L, V341I, A348T, N354D, S359N, V367F, K378R, R408I, Q409E, A435S, N439K, K458R, I472V, G476S, S477N, V483A, Y508H, H519P, etc., as compared to SEQ ID NO: 1) and spike protein variants (e.g., but not limited to D614G, as compared to SEQ ID NO: 1). For example, in some embodiments, the mRNA composition encoding SEQ ID NO: 7 (S P2) elicits an immune response against any one of a SARs-CoV-2 spike variant including RBD variants (e.g., but not limited to Q321L, V341I, A348T, N354D, S359N, V367F, K378R, R408I, Q409E, A435S, N439K, K458R, I472V, G476S, S477N, V483A, Y508H, H519P, etc., as compared to SEQ ID NO: 1) and spike protein variants (e.g., but not limited to D614G, as compared to SEQ ID NO: 1).

In some embodiments, mRNA compositions and/or methods described herein are characterized in that sera of vaccinated subjects display neutralizing activity against one or more SARs-CoV-2 spike variants including a mutation at position 501 in spike protein as compared to SEQ ID NO: 1. In some embodiments, mRNA compositions and/or methods described herein are characterized in that sera of vaccinated subjects display neutralizing activity against one or more SARs-CoV-2 spike variants including a N501Y mutation in spike protein as compared to SEQ ID NO: 1. Said one or more SARs-CoV-2 spike variants including a mutation at position 501 in spike protein as compared to SEQ ID NO: 1 or said one or more SARs-CoV-2 spike variants including a N501Y mutation in spike protein as compared to SEQ ID NO: 1 may include one or more further mutations as compared to SEQ ID NO: 1 (e.g., but not limited to H69/V70 deletion, Y144 deletion, A570D, D614G, P681H, T716I, S982A, D1118H, D80A, D215G, E484K, A701V, L18F, R246I, K417N, L242/A243/L244 deletion etc., as compared to SEQ ID NO: 1).

In particular embodiments, mRNA compositions and/or methods described herein are characterized in that sera of vaccinated subjects display neutralizing activity against SARs-CoV-2 spike variant “Variant of Concern 202012/01” (VOC-202012/01; also known as lineage B.1.1.7). The variant had previously been named the first Variant Under Investigation in December 2020 (VUI-202012/01) by Public Health England, but was reclassified to a Variant of Concern (VOC-202012/01). VOC-202012/01 is a variant of SARS-CoV-2 which was first detected in October 2020 during the COVID-19 pandemic in the United Kingdom from a sample taken the previous month, and it quickly began to spread by mid-December. It is correlated with a significant increase in the rate of COVID-19 infection in United Kingdom; this increase is thought to be at least partly because of change N501Y inside the spike glycoprotein's receptor-binding domain, which is needed for binding to ACE2 in human cells. The VOC-202012/01 variant is defined by 23 mutations: 13 non-synonymous mutations, 4 deletions, and 6 synonymous mutations (i.e., there are 17 mutations that change proteins and six that do not). The spike protein changes in VOC 202012/01 include deletion 69-70, deletion 144, N501Y, A570D, D614G, P681H, T716I, S982A, and D1118H. One of the most important changes in VOC-202012/01 seems to be N501Y, a change from asparagine (N) to tyrosine (Y) at amino-acid site 501. This mutation alone or in combination with the deletion at positions 69/70 in the N terminal domain (NTD) may enhance the transmissibility of the virus.

In particular embodiments, mRNA compositions and/or methods described herein are characterized in that sera of vaccinated subjects display neutralizing activity against SARs-CoV-2 spike variant including the following mutations: deletion 69-70, deletion 144, N501Y, A570D, D614G, P681H, T716I, S982A, and D1118H as compared to SEQ ID NO: 1.

In particular embodiments, mRNA compositions and/or methods described herein are characterized in that sera of vaccinated subjects display neutralizing activity against SARs-CoV-2 spike variant “501.V2”. This variant was first observed in samples from October 2020, and since then more than 300 cases with the 501.V2 variant have been confirmed by whole genome sequencing (WGS) in South Africa, where in December 2020 it was the dominant form of the virus. Preliminary results indicate that this variant may have an increased transmissibility. The 501.V2 variant is defined by multiple spike protein changes including: D80A, D215G, E484K, N501Y and A701V, and more recently collected viruses have additional changes: L18F, R246I, K417N, and deletion 242-244.

In particular embodiments, mRNA compositions and/or methods described herein are characterized in that sera of vaccinated subjects display neutralizing activity against SARs-CoV-2 spike variant including the following mutations: D80A, D215G, E484K, N501Y and A701V as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

In some embodiments, mRNA compositions and/or methods described herein are characterized in that sera of vaccinated subjects display neutralizing activity against one or more SARs-CoV-2 spike variants including a H69/V70 deletion in spike protein as compared to SEQ ID NO: 1.

In some embodiments, one or more SARs-CoV-2 spike variants including a H69/V70 deletion in spike protein as compared to SEQ ID NO: 1 may include one or more further mutations as compared to SEQ ID NO: 1 (e.g., but not limited to Y144 deletion, N501Y, A570D, D614G, P681H, T716I, S982A, D1118H, D80A, D215G, E484K, A701V, L18F, R246I, K417N, L242/A243/L244 deletion, Y453F, I692V, S1147L, M1229I etc., as compared to SEQ ID NO: 1),

In particular embodiments, mRNA compositions and/or methods described herein are characterized in that sera of vaccinated subjects display neutralizing activity against SARs-CoV-2 spike variant “Variant of Concern 202012/01” (VOC-202012/01; also known as lineage B.1.1.7).

In particular embodiments, mRNA compositions and/or methods described herein are characterized in that sera of vaccinated subjects display neutralizing activity against SARs-CoV-2 spike variant including the following mutations: deletion 69-70, deletion 144, N501Y, A570D, D614G, P681H, T716I, S982A, and D1118H as compared to SEQ ID NO: 1.

In particular embodiments, mRNA compositions and/or methods described herein are characterized in that sera of vaccinated subjects display neutralizing activity against SARs-CoV-2 spike variant “Cluster 5”, also referred to as ΔFVI-spike by the Danish State Serum Institute (SSI). It was discovered in North Jutland, Denmark, and is believed to have been spread from minks to humans via mink farms. In cluster 5, several different mutations in the spike protein of the virus have been confirmed. The specific mutations include 69-70deltaHV (a deletion of the histidine and valine residues at the 69th and 70th position in the protein), Y453F (a change from tyrosine to phenylalanine at position 453), I692V (isoleucine to valine at position 692), M1229I (methionine to isoleucine at position 1229), and optionally S1147L (serine to leucine at position 1147).

In particular embodiments, mRNA compositions and/or methods described herein are characterized in that sera of vaccinated subjects display neutralizing activity against SARs-CoV-2 spike variant including the following mutations: deletion 69-70, Y453F, I692V, M1229I, and optionally S1147L, as compared to SEQ ID NO: 1.

In some embodiments, mRNA compositions and/or methods described herein are characterized in that sera of vaccinated subjects display neutralizing activity against one or more SARs-CoV-2 spike variants including a mutation at position 614 in spike protein as compared to SEQ ID NO: 1. In some embodiments, mRNA compositions and/or methods described herein are characterized in that sera of vaccinated subjects display neutralizing activity against one or more SARs-CoV-2 spike variants including a D614G mutation in spike protein as compared to SEQ ID NO: 1. In some embodiments, one or more SARs-CoV-2 spike variants including a mutation at position 614 in spike protein as compared to SEQ ID NO: 1 or said one or more SARs-CoV-2 spike variants including a D614G mutation in spike protein as compared to SEQ ID NO: 1 may include one or more further mutations as compared to SEQ ID NO: 1 (e.g., but not limited to H69/V70 deletion, Y144 deletion, N501Y, A570D, P681H, T716I, S982A, D1118H, D80A, D215G, E484K, A701V, L18F, R246I, K417N, L242/A243/L244 deletion, Y453F, I692V, S1147L, M1229I etc., as compared to SEQ ID NO: 1).

In particular embodiments, mRNA compositions and/or methods described herein are characterized in that sera of vaccinated subjects display neutralizing activity against SARs-CoV-2 spike variant “Variant of Concern 202012/01” (VOC-202012/01; also known as lineage B.1.1.7).

In particular embodiments, mRNA compositions and/or methods described herein are characterized in that sera of vaccinated subjects display neutralizing activity against SARs-CoV-2 spike variant including the following mutations: deletion 69-70, deletion 144, N501Y, A570D, D614G, P681H, T716I, S982A, and D1118H as compared to SEQ ID NO: 1.

In particular embodiments, mRNA compositions and/or methods described herein are characterized in that sera of vaccinated subjects display neutralizing activity against SARs-CoV-2 spike variant including the following mutations: D80A, D215G, E484K, N501Y, A701V, and D614G as compared to SEQ ID NO: 1, and optionally: L18F, R246I, K417N, and deletion 242-244 as compared to SEQ ID NO: 1.

In some embodiments, mRNA compositions and/or methods described herein are characterized in that sera of vaccinated subjects display neutralizing activity against one or more SARs-CoV-2 spike variants including a mutation at positions 501 and 614 in spike protein as compared to SEQ ID NO: 1. In some embodiments, mRNA compositions and/or methods described herein are characterized in that sera of vaccinated subjects display neutralizing activity against one or more SARs-CoV-2 spike variants including a N501Y mutation and a D614G mutation in spike protein as compared to SEQ ID NO: 1.

In some embodiments, one or more SARs-CoV-2 spike variants including a mutation at positions 501 and 614 in spike protein as compared to SEQ ID NO: 1 or said one or more SARs-CoV-2 spike variants including a N501Y mutation and a D614G mutation in spike protein as compared to SEQ ID NO: 1 may include one or more further mutations as compared to SEQ ID NO: 1 (e.g., but not limited to H69/V70 deletion, Y144 deletion, A570D, P681H, T716I, S982A, D1118H, D80A, D215G, E484K, A701V, L18F, R246I, K417N, L242/A243/L244 deletion, Y453F, I692V, S1147L, M1229I etc., as compared to SEQ ID NO: 1).

In particular embodiments, mRNA compositions and/or methods described herein are characterized in that sera of vaccinated subjects display neutralizing activity against SARs-CoV-2 spike variant “Variant of Concern 202012/01” (VOC-202012/01; also known as lineage B.1.1.7).

In particular embodiments, mRNA compositions and/or methods described herein are characterized in that sera of vaccinated subjects display neutralizing activity against SARs-CoV-2 spike variant including the following mutations: deletion 69-70, deletion 144, N501Y, A570D, D614G, P681H, T716I, S982A, and D1118H as compared to SEQ ID NO: 1.

In particular embodiments, mRNA compositions and/or methods described herein are characterized in that sera of vaccinated subjects display neutralizing activity against SARs-CoV-2 spike variant including the following mutations: D80A, D215G, E484K, N501Y, A701V, and D614G as compared to SEQ ID NO: 1, and optionally: L18F, R246I, K417N, and deletion 242-244 as compared to SEQ ID NO: 1.

In some embodiments, mRNA compositions and/or methods described herein are characterized in that sera of vaccinated subjects display neutralizing activity against one or more SARs-CoV-2 spike variants including a mutation at position 484 in spike protein as compared to SEQ ID NO: 1. In some embodiments, mRNA compositions and/or methods described herein are characterized in that sera of vaccinated subjects display neutralizing activity against one or more SARs-CoV-2 spike variants including a E484K mutation in spike protein as compared to SEQ ID NO: 1. In some embodiments, one or more SARs-CoV-2 spike variants including a mutation at position 484 in spike protein as compared to SEQ ID NO: 1 or said one or more SARs-CoV-2 spike variants including a E484K mutation in spike protein as compared to SEQ ID NO: 1 may include one or more further mutations as compared to SEQ ID NO: 1 (e.g., but not limited to H69/V70 deletion, Y144 deletion, N501Y, A570D, D614G, P681H, T716I, S982A, D1118H, D80A, D215G, A701V, L18F, R246I, K417N, L242/A243/L244 deletion, Y453F, I692V, S1147L, M1229I, T20N, P26S, D138Y, R190S, K417T, H655Y, T1027I, V1176F etc., as compared to SEQ ID NO: 1).

In particular embodiments, mRNA compositions and/or methods described herein are characterized in that sera of vaccinated subjects display neutralizing activity against SARs-CoV-2 spike variant “501.V2”.

In particular embodiments, mRNA compositions and/or methods described herein are characterized in that sera of vaccinated subjects display neutralizing activity against SARs-CoV-2 spike variant including the following mutations: D80A, D215G, E484K, N501Y, and A701V, as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

Lineage B.1.1.248, known as the Brazil(ian) variant, is one of the variants of SARS-CoV-2 which has been named P.1 lineage and has 17 unique amino acid changes, 10 of which in its spike protein, including N501Y and E484K. B.1.1.248 originated from B.1.1.28. E484K is present in both B.1.1.28 and B.1.1.248. B.1.1.248 has a number of S-protein polymorphisms [L18F, T20N, P26S, D138Y, R190S, K417T, E484K, N501Y, H655Y, T1027I, V1176F] and is similar in certain key RBD positions (K417, E484, N501) to variant described from South Africa.

In particular embodiments, mRNA compositions and/or methods described herein are characterized in that sera of vaccinated subjects display neutralizing activity against SARs-CoV-2 spike variant “B.1.1.28”.

In particular embodiments, mRNA compositions and/or methods described herein are characterized in that sera of vaccinated subjects display neutralizing activity against SARs-CoV-2 spike variant “B.1.1.248”.

In particular embodiments, mRNA compositions and/or methods described herein are characterized in that sera of vaccinated subjects display neutralizing activity against SARs-CoV-2 spike variant including the following mutations: L18F, T20N, P26S, D138Y, R190S, K417T, E484K, N501Y, H655Y, T1027I, and V1176F as compared to SEQ ID NO: 1.

In some embodiments, mRNA compositions and/or methods described herein are characterized in that sera of vaccinated subjects display neutralizing activity against one or more SARs-CoV-2 spike variants including a mutation at positions 501 and 484 in spike protein as compared to SEQ ID NO: 1. In some embodiments, mRNA compositions and/or methods described herein are characterized in that sera of vaccinated subjects display neutralizing activity against one or more SARs-CoV-2 spike variants including a N501Y mutation and a E484K mutation in spike protein as compared to SEQ ID NO: 1.

In some embodiments, one or more SARs-CoV-2 spike variants including a mutation at positions 501 and 484 in spike protein as compared to SEQ ID NO: 1 or said one or more SARs-CoV-2 spike variants including a N501Y mutation and a E484K mutation in spike protein as compared to SEQ ID NO: 1 may include one or more further mutations as compared to SEQ ID NO: 1 (e.g., but not limited to H69/V70 deletion, Y144 deletion, A570D, D614G, P681H, T716I, S982A, D1118H, D80A, D215G, A701V, L18F, R246I, K417N, L242/A243/L244 deletion, Y453F, I692V, S1147L, M1229I, T20N, P26S, D138Y, R190S, K417T, H655Y, T1027I, V1176F etc., as compared to SEQ ID NO: 1).

In particular embodiments, mRNA compositions and/or methods described herein are characterized in that sera of vaccinated subjects display neutralizing activity against SARs-CoV-2 spike variant “501.V2”.

In particular embodiments, mRNA compositions and/or methods described herein are characterized in that sera of vaccinated subjects display neutralizing activity against SARs-CoV-2 spike variant including the following mutations: D80A, D215G, E484K, N501Y and A701V as compared to SEQ ID NO: 1, and optionally: L18F, R246I, K417N, and deletion 242-244 as compared to SEQ ID NO: 1. Said SARs-CoV-2 spike variant may also include a D614G mutation as compared to SEQ ID NO: 1.

In particular embodiments, mRNA compositions and/or methods described herein are characterized in that sera of vaccinated subjects display neutralizing activity against SARs-CoV-2 spike variant “B.1.1.248”.

In particular embodiments, mRNA compositions and/or methods described herein are characterized in that sera of vaccinated subjects display neutralizing activity against SARs-CoV-2 spike variant including the following mutations: L18F, T20N, P26S, D138Y, R190S, K417T, E484K, N501Y, H655Y, T1027I, and V1176F as compared to SEQ ID NO: 1.

In some embodiments, mRNA compositions and/or methods described herein are characterized in that sera of vaccinated subjects display neutralizing activity against one or more SARs-CoV-2 spike variants including a mutation at positions 501, 484 and 614 in spike protein as compared to SEQ ID NO: 1. In some embodiments, mRNA compositions and/or methods described herein are characterized in that sera of vaccinated subjects display neutralizing activity against one or more SARs-CoV-2 spike variants including a N501Y mutation, a E484K mutation and a D614G mutation in spike protein as compared to SEQ ID NO: 1.

In some embodiments, one or more SARs-CoV-2 spike variants including a mutation at positions 501, 484 and 614 in spike protein as compared to SEQ ID NO: 1 or said one or more SARs-CoV-2 spike variants including a N501Y mutation, a E484K mutation and a D614G mutation in spike protein as compared to SEQ ID NO: 1 may include one or more further mutations as compared to SEQ ID NO: 1 (e.g., but not limited to H69/V70 deletion, Y144 deletion, A570D, P681H, T716I, S982A, D1118H, D80A, D215G, A701V, L18F, R246I, K417N, L242/A243/L244 deletion, Y453F, I692V, S1147L, M1229I, T20N, P26S, D138Y, R190S, K417T, H655Y, T1027I, V1176F etc., as compared to SEQ ID NO: 1).

In particular embodiments, mRNA compositions and/or methods described herein are characterized in that sera of vaccinated subjects display neutralizing activity against SARs-CoV-2 spike variant including the following mutations: D80A, D215G, E484K, N501Y, A701V, and D614G as compared to SEQ ID NO: 1, and optionally: L18F, R246I, K417N, and deletion 242-244 as compared to SEQ ID NO: 1.

In some embodiments, mRNA compositions and/or methods described herein are characterized in that sera of vaccinated subjects display neutralizing activity against one or more SARs-CoV-2 spike variants including a L242/A243/L244 deletion in spike protein as compared to SEQ ID NO: 1.

In some embodiments, one or more SARs-CoV-2 spike variants including a L242/A243/L244 deletion in spike protein as compared to SEQ ID NO: 1 may include one or more further mutations as compared to SEQ ID NO: 1 (e.g., but not limited to H69/V70 deletion, Y144 deletion, N501Y, A570D, D614G, P681H, T716I, S982A, D1118H, D80A, D215G, E484K, A701V, L18F, R246I, K417N, Y453F, I692V, S1147L, M1229I, T20N, P26S, D138Y, R190S, K417T, H655Y, T1027I, V1176F etc., as compared to SEQ ID NO: 1).

In particular embodiments, mRNA compositions and/or methods described herein are characterized in that sera of vaccinated subjects display neutralizing activity against SARs-CoV-2 spike variant “501.V2”.

In particular embodiments, mRNA compositions and/or methods described herein are characterized in that sera of vaccinated subjects display neutralizing activity against SARs-CoV-2 spike variant including the following mutations: D80A, D215G, E484K, N501Y, A701V and deletion 242-244 as compared to SEQ ID NO: 1, and optionally: L18F, R246I, and K417N, as compared to SEQ ID NO: 1. Said SARs-CoV-2 spike variant may also include a D614G mutation as compared to SEQ ID NO: 1.

In some embodiments, mRNA compositions and/or methods described herein are characterized in that sera of vaccinated subjects display neutralizing activity against one or more SARs-CoV-2 spike variants including a mutation at position 417 in spike protein as compared to SEQ ID NO: 1. In some embodiments, mRNA compositions and/or methods described herein are characterized in that sera of vaccinated subjects display neutralizing activity against one or more SARs-CoV-2 spike variants including a K417N or K417T mutation in spike protein as compared to SEQ ID NO: 1.

In some embodiments, one or more SARs-CoV-2 spike variants including a mutation at position 417 in spike protein as compared to SEQ ID NO: 1 or said one or more SARs-CoV-2 spike variants including a K417N or K417T mutation in spike protein as compared to SEQ ID NO: 1 may include one or more further mutations as compared to SEQ ID NO: 1 (e.g., but not limited to H69/V70 deletion, Y144 deletion, N501Y, A570D, D614G, P681H, T716I, S982A, D1118H, D80A, D215G, E484K, A701V, L18F, R246I, L242/A243/L244 deletion, Y453F, I692V, S1147L, M1229I, T20N, P26S, D138Y, R190S, H655Y, T1027I, V1176F etc., as compared to SEQ ID NO: 1).

In particular embodiments, mRNA compositions and/or methods described herein are characterized in that sera of vaccinated subjects display neutralizing activity against SARs-CoV-2 spike variant “501.V2”.

In particular embodiments, mRNA compositions and/or methods described herein are characterized in that sera of vaccinated subjects display neutralizing activity against SARs-CoV-2 spike variant including the following mutations: D80A, D215G, E484K, N501Y, A701V and K417N, as compared to SEQ ID NO: 1, and optionally: L18F, R246I, and deletion 242-244 as compared to SEQ ID NO: 1. Said SARs-CoV-2 spike variant may also include a D614G mutation as compared to SEQ ID NO: 1.

In particular embodiments, mRNA compositions and/or methods described herein are characterized in that sera of vaccinated subjects display neutralizing activity against SARs-CoV-2 spike variant “B.1.1.248”.

In particular embodiments, mRNA compositions and/or methods described herein are characterized in that sera of vaccinated subjects display neutralizing activity against SARs-CoV-2 spike variant including the following mutations: L18F, T20N, P26S, D138Y, R190S, K417T, E484K, N501Y, H655Y, T1027I, and V1176F as compared to SEQ ID NO: 1.

In some embodiments, mRNA compositions and/or methods described herein are characterized in that sera of vaccinated subjects display neutralizing activity against one or more SARs-CoV-2 spike variants including a mutation at positions 417 and 484 and/or 501 in spike protein as compared to SEQ ID NO: 1. In some embodiments, mRNA compositions and/or methods described herein are characterized in that sera of vaccinated subjects display neutralizing activity against one or more SARs-CoV-2 spike variants including a K417N or K417T mutation and a E484K and/or N501Y mutation in spike protein as compared to SEQ ID NO: 1.

In some embodiments, one or more SARs-CoV-2 spike variants including a mutation at positions 417 and 484 and/or 501 in spike protein as compared to SEQ ID NO: 1 or said one or more SARs-CoV-2 spike variants including a K417N or K417T mutation and a E484K and/or N501Y mutation in spike protein as compared to SEQ ID NO: 1 may include one or more further mutations as compared to SEQ ID NO: 1 (e.g., but not limited to H69/V70 deletion, Y144 deletion, A570D, D614G, P681H, T716I, S982A, D1118H, D80A, D215G, A701V, L18F, R246I, L242/A243/L244 deletion, Y453F, I692V, S1147L, M1229I, T20N, P26S, D138Y, R190S, H655Y, T1027I, V1176F etc., as compared to SEQ ID NO: 1).

In particular embodiments, mRNA compositions and/or methods described herein are characterized in that sera of vaccinated subjects display neutralizing activity against SARs-CoV-2 spike variant “501.V2”.

In particular embodiments, mRNA compositions and/or methods described herein are characterized in that sera of vaccinated subjects display neutralizing activity against SARs-CoV-2 spike variant including the following mutations: D80A, D215G, E484K, N501Y, A701V and K417N, as compared to SEQ ID NO: 1, and optionally: L18F, R246I, and deletion 242-244 as compared to SEQ ID NO: 1. Said SARs-CoV-2 spike variant may also include a D614G mutation as compared to SEQ ID NO: 1.

In particular embodiments, mRNA compositions and/or methods described herein are characterized in that sera of vaccinated subjects display neutralizing activity against SARs-CoV-2 spike variant “B.1.1.248”.

In particular embodiments, mRNA compositions and/or methods described herein are characterized in that sera of vaccinated subjects display neutralizing activity against SARs-CoV-2 spike variant including the following mutations: L18F, T20N, P26S, D138Y, R190S, K417T, E484K, N501Y, H655Y, T1027I, and V1176F as compared to SEQ ID NO: 1.

In some embodiments, mRNA compositions and/or methods described herein are characterized in that sera of vaccinated subjects display neutralizing activity against SARs-CoV-2 spike variant of the Omicron (B.1.1.529) variant. Omicron (B.1.1.529) variant is a variant of SARS-CoV-2 which was detected in South Africa. Multiple Omicron sublineages have arisen, including e.g., the BA.1, BA.2, BA.2.12.1, BA.3, BA.4, BA.5, and BA.2.75 sublineages. As used herein, unless otherwise specified, “Omicron variant” refers to a SARS-CoV-2 variant having one or mutations characteristic of BA.1 or any variant thereof that has since arisen (including, but not limited to, e.g., BA.2, BA.2.12.1, BA.2.12.1, BA.4 or BA.5, BA.2.75, BA.2.75.2, BJ.1, BA.4.6 or BF.7, XBB, XBB.1, XBB.2, XBB.1.3, BA.2.3.20, BQ.1.1, as described herein). In some embodiments, the spike protein changes in Omicron (B.1.1.529) BA.1 variant include A67V, Δ69-70, T95I, G142D, Δ143-145, Δ211, L212I, ins214EPE (insertion of EPE following amino acid 214), G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In some embodiments, the spike protein changes in Omicron (B.1.1.529) variant include A67V, Δ69-70, T95I, G142D, Δ143-145, Δ211, L212I, ins214EPE (insertion of EPE following amino acid 214), G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F. In some embodiments, the spike changes in Omicron BA.2 variant include T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K. In some embodiments BA.4 and BA.5 have the same Spike protein amino acid sequence, in which case “BA.4/5” is used to either Omicron variant. In some embodiments, the spike changes in Omicron BA.4/5 include: T19I, Δ24-26, A27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, and N969K. In some embodiments, the spike changes in Omicron BA.2.75 include T19I, Δ24-26, A27S, G142D, K147E, W152R, F157L, I210V, V213G, G257S, G339H, N354D, S371F, S373P, S375F, T376A, D405N, R408S, K417N, N440K, G446S, N460K, S477N, T478K, E484A, Q498R, N501Y, Y505H D614G, H655Y, N679K, P681H, N764K, D796Y, Q954H, and N969K.

In some embodiments, mRNA compositions and/or methods described herein are characterized in that sera of vaccinated subjects display neutralizing activity against SARs-CoV-2 spike variant including at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37 of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, N501Y, S375F, Y505H, V143del, H69del, V70del, N211del, L212I, ins214EPE, G142D, Y144del, Y145del, L141del, Y144F, Y145D, G142del, as compared to SEQ ID NO: 1.

In some embodiments, mRNA compositions and/or methods described herein are characterized in that sera of vaccinated subjects display neutralizing activity against SARs-CoV-2 spike variant including at least 10, at least 15, at least 20, at least 21, at least 22, at least 23, at least 24, or all of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, as compared to SEQ ID NO: 1. Said SARs-CoV-2 spike variant may include at least 1, at least 2, at least 3, at least 4, at least 5, or all of the following mutations: N501Y, S375F, Y505H, V143del, H69del, V70del, as compared to SEQ ID NO: 1, and/or may include at least 1, at least 2, at least 3, at least 4, at least 5, or all of the following mutations: N211del, L212I, ins214EPE, G142D, Y144del, Y145del, as compared to SEQ ID NO: 1. In some embodiments, said SARs-CoV-2 spike variant may include at least 1, at least 2, at least 3, or all of the following mutations: L141del, Y144F, Y145D, G142del, as compared to SEQ ID NO: 1.

In some embodiments, mRNA compositions and/or methods described herein are characterized in that sera of vaccinated subjects display neutralizing activity against SARs-CoV-2 spike variant including at least 10, at least 15, at least 20, at least 21, at least 22, at least 23, at least 24, at least 25, at least 26, at least 27, at least 28, at least 29, at least 30, at least 31, at least 32, at least 33 of the following mutations: A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, mRNA compositions and/or methods described herein are characterized in that sera of vaccinated subjects display neutralizing activity against a SARS-CoV-2 spike variant including at least 10, at least 15, at least 20, at least 21, at least 22, at least 23, at least 24, at least 25, at least 26, at least 27, at least 28, at least 29, at least 30, or at least 31, of the following mutations: T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, as compared to SEQ ID NO: 1.

In some embodiments, mRNA compositions and/or methods described herein are characterized in that sera of vaccinated subjects display neutralizing activity against a SARS-CoV-2 spike variant including at least 10, at least 15, at least 20, at least 21, at least 22, at least 23, at least 24, at least 25, at least 26, at least 27, at least 28, at least 29, at least 30, at least 31, at least 32, at least 33, or at least 34 of the following mutations: T19I, Δ24-26, A275, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, and N969K, as compared to SEQ ID NO: 1.

In some embodiments, mRNA compositions and/or methods described herein are characterized in that sera of vaccinated subjects display neutralizing activity against SARs-CoV-2 spike variant including the following mutations: A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, mRNA compositions and/or methods described herein are characterized in that sera of vaccinated subjects display neutralizating against a SARS-CoV-2 spike variant including the following mutations: T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K. In some embodiments, mRNA compositions and/or methods described herein are characterized in that sera of vaccinated subjects display neutralizating against SARS-CoV2 spike variant including the following mutations: T19I, Δ24-26, A27S, 469/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, and N969K, as compared to SEQ ID NO: 1.

In some embodiments, mRNA compositions and/or methods described herein are characterized in that sera of vaccinated subjects display neutralizing activity against SARs-CoV-2 spike variant including the following mutations: A67V, Δ69-70, T95I, G142D, Δ143-145, Δ211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, mRNA compositions and/or methods described herein are characterized in that sera of vaccinated subjects display neutralizing activity against SARs-CoV-2 spike variant including the following mutations: T19I, Δ24-26, A27S, G142D, K147E, W152R, F157L, I210V, V213G, G257S, G339H, N354D, S371F, S373P, S375F, T376A, D405N, R408S, K417N, N440K, G446S, N460K, S477N, T478K, E484A, Q498R, N501Y, Y505H D614G, H655Y, N679K, P681H, N764K, D796Y, Q954H, and N969K, as compared to SEQ ID NO: 1.

The SARs-CoV-2 spike variants described herein may or may not include a D614G mutation as compared to SEQ ID NO: 1.

In some embodiments, SARS-CoV-2 spike variants described herein comprise a mutation in a furin cleavage site (e.g., in some embodiments residues 682-685 of SEQ ID NO: 1). In some embodiments, a SARS-CoV-2 spike variant comprises a mutation in the furin cleavage site that prevents cleavage by a furin protease (e.g., a human furin protease). In some embodiments, a SARS-CoV-2 variant described herein comprises a furin mutation disclosed in WO2021163365 or WO2021243122 (e.g., a GSAS mutation), the contents of both of which are incorporated by reference herein in their entirety.

In some embodiments, mRNA compositions and/or methods described herein can provide protection against SARS-CoV-2 and/or influenza virus (e.g., influenza type A and/or type B viruses), and/or decrease severity of SARS-CoV-2 infection and/or influenza virus infection (e.g. influenza type A and/or type B virus infection) in at least 50% of subjects receiving such mRNA compositions and/or methods. In some embodiments, compositions disclosed herein can be used for active immunization to prevent both SARS-CoV-2 infection and Influenza subtype A and subtype B infection in individuals (e.g., in pediatric patients, in pregnant patients, and in patients 18 years of age or older).

In some embodiments, populations to be treated with mRNA compositions described herein include subjects 18 years of age and older. In some embodiments, populations to be treated with mRNA compositions described herein include subjects of age 18-55. In some embodiments, populations to be treated with mRNA compositions described herein include subjects of age 56-85. In some embodiments, populations to be treated with mRNA compositions described herein include older subjects (e.g., over age 60, 65, 70, 75, 80, 85, etc, for example subjects of age 65-85). In some embodiments, populations to be treated with mRNA compositions described herein include subjects of age 18-85. In some embodiments, populations to be treated with mRNA compositions described herein include subjects of age 18 or younger. In some embodiments, populations to be treated with mRNA compositions described herein include subjects of age 12 or younger. In some embodiments, populations to be treated with mRNA compositions described herein include subjects of age 10 or younger. In some embodiments, populations to be treated with mRNA compositions described herein may include adolescent populations (e.g., individuals approximately 12 to approximately 17 years of age). In some embodiments, populations to be treated with mRNA compositions described herein may include pediatric populations (e.g., as described herein). In some embodiments, populations to be treated with mRNA compositions described herein include infants (e.g., less than 1 year old). In some embodiments, populations to be treated with mRNA compositions described herein do not include infants (e.g., less than 1 year) whose mothers have received such mRNA compositions described herein during pregnancy. Without wishing to be bound by any particular theory, a rat study has suggested that a SARS-CoV-2 neutralizing antibody response induced in female rats given such mRNA compositions during pregnancy can pass onto fetuses. In some embodiments, populations to be treated with mRNA compositions described herein include infants (e.g., less than 1 year) whose mothers did not receive such mRNA compositions described herein during pregnancy. In some embodiments, populations to be treated with mRNA compositions described herein may include pregnant women; in some embodiments, infants whose mothers were vaccinated during pregnancy (e.g., who received at least one dose, or alternatively only who received both doses), are not vaccinated during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth. Alternatively or additionally, in some embodiments, infants whose mothers were vaccinated during pregnancy (e.g., who received at least one dose, or alternatively only who received both doses), receive reduced vaccination (e.g., lower doses and/or smaller numbers of administrations—e.g., boosters—and/or lower total exposure over a given period of time) after birth, for example during the first weeks, months, or even years (e.g., 1, 2, 3, 4, 5, 6, 7, 8 weeks or more, or 1, 2, 3, 4, 5, 6, 7, 8, 9, 10, 11, 12, 13, 14, 15, 16, 17, 18, 19, 20, 21, 22, 23, 24 months or more, or 1, 2, 3, 4, 5 years or more) post-birth or may need reduced vaccination (e.g., lower doses and/or smaller numbers of administrations—e.g., boosters—over a given period of time), In some embodiments, compositions as provided herein are administered to populations that do not include pregnant women.

In some particular embodiments, compositions as provided herein are administered to pregnant women according to a regimen that includes a first dose administered after about 24 weeks of gestation (e.g., after about 22, 23, 24, 25, 26, 27, 28 or more weeks of gestation); in some embodiments, compositions as provided herein are administered to pregnant women according to a regimen that includes a first dose administered before about 34 weeks of gestation (e.g., before about 30, 31, 32, 33, 34, 35, 36, 37, 38 weeks of gestation). In some embodiments, compositions as provided herein are administered to pregnant women according to a regimen that includes a first dose administered after about 24 weeks (e.g., after about 27 weeks of gestation, e.g., between about 24 weeks and 34 weeks, or between about 27 weeks and 34 weeks) of gestation and a second dose administered about 21 days later; in some embodiments both doses are administered prior to delivery. Without wishing to be bound by any particular theory, it is proposed that such a regimen (e.g., involving administration of a first dose after about 24 weeks, or 27 weeks of gestation and optionally before about 34 weeks of gestation), and optionally a second dose within about 21 days, ideally before delivery, may have certain advantages in terms of safety (e.g., reduced risk of premature delivery or of fetal morbidity or mortality) and/or efficacy (e.g., carryover vaccination imparted to the infant) relative to alternative dosing regimens (e.g., dosing at any time during pregnancy, refraining from dosing during pregnancy, and/or dosing later in pregnancy for example so that only one dose is administered during gestation. In some embodiments, infants born of mothers vaccinated during pregnancy, e.g., according to a particular regimen as described herein, may not need further vaccination, or may need reduced vaccination (e.g., lower doses and/or smaller numbers of administrations—e.g., boosters—, and/or lower overall exposure over a given period of time), for a period of time (e.g., as noted herein) after birth.

In some embodiments, compositions as provided herein are administered to populations in which women are advised against becoming pregnant for a period of time after receipt of the vaccine (e.g., after receipt of a first dose of the vaccine, after receipt of a final dose of the vaccine, etc.); in some such embodiments, the period of time may be at least 1 week, at least 2 weeks, at least 3 weeks, at least 4 weeks, at least 5 weeks, at least 6 weeks, at least 7 weeks, at least 8 weeks, at least 9 weeks, at least 10 weeks or more, or may be at least 1 month, at least 2 months, at least 3 months, at least 4 months, at least 5 months, at least 6 months, or more.

In some embodiments, populations to be treated with mRNA compositions described herein may include one or more populations with one or more particularly high risk conditions or history, e.g., as noted herein. For example, in some embodiments, populations to be treated with mRNA compositions described herein may include subjects whose profession and/or environmental exposure may dramatically increase their risk of getting SARS-CoV-2 infection and/or influenza virus infection (including, e.g., but not limited to mass transportation, prisoners, grocery store workers, residents in long-term care facilities, butchers or other meat processing workers, healthcare workers, and/or first responders, e.g., emergency responders). In particular embodiments, populations to be treated with mRNA compositions described herein may include healthcare workers and/or first responders, e.g., emergency responders. In some embodiments, populations to be treated with mRNA compositions described herein may include those with a history of smoking or vaping (e.g., within 6 months, 12 months or more, including a history of chronic smoking or vaping). In some embodiments, populations to be treated with mRNA compositions described herein may include certain ethnic groups that have been determined to be more susceptible to SARS-CoV-2 infection and/or influenza virus infection.

In some embodiments, populations to be treated with mRNA compositions described herein may include certain populations with a blood type that may have been determined to more susceptible to SARS-CoV-2 infection and/or influenza virus infection. In some embodiments, populations to be treated with mRNA compositions described herein may include immunocompromised subjects (e.g., those with HIV/AIDS; cancer patients (e.g., receiving antitumor treatment); patients who are taking certain immunosuppressive drugs (e.g., transplant patients, cancer patients, etc.); autoimmune diseases or other physiological conditions expected to warrant immunosuppressive therapy (e.g., within 3 months, within 6 months, or more); and those with inherited diseases that affect the immune system (e.g., congenital agammaglobulinemia, congenital IgA deficiency)). In some embodiments, populations to be treated with mRNA compositions described herein may include those with an infectious disease. For example, in some embodiments, populations to be treated with mRNA compositions described herein may include those infected with human immunodeficiency virus (HIV) and/or a hepatitis virus (e.g., HBV, HCV). In some embodiments, populations to be treated with mRNA compositions described herein may include those with underlying medical conditions. Examples of such underlying medical conditions may include, but are not limited to hypertension, cardiovascular disease, diabetes, chronic respiratory disease, e.g., chronic pulmonary disease, asthma, etc., cancer, and other chronic diseases such as, e.g., lupus, rheumatoid arthritis, chronic liver diseases, chronic kidney diseases (e.g., Stage 3 or worse such as in some embodiments as characterized by a glomerular filtration rate (GFR) of less than 60 mL/min/1.73 m²). In some embodiments, populations to be treated with mRNA compositions described herein may include overweight or obese subjects, e.g., specifically including those with a body mass index (BMI) above about 30 kg/m². In some embodiments, populations to be treated with mRNA compositions described herein may include subjects who have prior diagnosis of COVID-19 or evidence of current or prior SARS-CoV-2 infection, e.g., based on serology or nasal swab. In some embodiments, populations to be treated include white and/or non-Hispanic/non-Latino.

In some embodiments, certain mRNA compositions described herein may be selected for administration to Asian populations (e.g., Chinese populations), or in particular embodiments to older Asian populations (e.g., 60 years old or over, e.g., 60-85 or 65-85 years old).

In some embodiments, an mRNA composition as provided herein is administered to and/or assessed in subject(s) who have been determined not to show evidence of prior infection, and/or of present infection, before administration; in some embodiments, evidence of prior infection and/or of present infection, may be or include evidence of intact virus, or any viral nucleic acid, protein, lipid etc. present in the subject (e.g., in a biological sample thereof, such as blood, cells, mucus, and/or tissue), and/or evidence of a subject's immune response to the same. In some embodiments, an mRNA composition as provided herein is administered to and/or assessed in subject(s) who have been determined to show evidence of prior infection, and/or of present infection, before administration; in some embodiments, evidence of prior infection and/or of present infection, may be or include evidence of intact virus, or any viral nucleic acid, protein, lipid etc. present in the subject (e.g., in a biological sample thereof, such as blood, cells, mucus, and/or tissue), and/or evidence of a subject's immune response to the same. In some embodiments, a subject is considered to have a prior infection based on having a positive N-binding antibody test result or positive nucleic acid amplification test (NAAT) result on the day of Dose 1.

In some embodiments, an RNA (e.g., mRNA) composition as provided herein is administered to a subject who has been informed of a risk of side effects that may include one or more of, for example: chills, fever, headache, injection site pain, muscle pain, tiredness; in some embodiments, an RNA (e.g., mRNA) composition is administered to a subject who has been invited to notify a healthcare provider if one or more such side effects occurs, is experienced as more than mild or moderate, persists for a period of more than a day or a few days, or if any serious or unexpected event is experienced that the subject reasonably considers may be associated with receipt of the composition. In some embodiments, an RNA (e.g., mRNA) composition as provided herein is administered to a subject who has been invited to notify a healthcare provider of particular medical conditions which may include, for example, one or more of allergies, bleeding disorder or taking a blood thinner medication, breastfeeding, fever, immunocompromised state or taking medication that affects the immune system, pregnancy or plan to become pregnant, etc. In some embodiments, an RNA (e.g., mRNA) composition as provided herein is administered to a subject who has been invited to notify a healthcare provider of having received another COVID-19 vaccine and/or another influenza vaccine (e.g., COVID-19 or influenza vaccines described herein). In some embodiments, an RNA (e.g., mRNA) composition as provided herein is administered to a subject not having one of the following medical conditions: experiencing febrile illness, receiving immunosuppressant therapy (e.g., receiving a known immunosuppressive medication or radiotherapy within the past 60 days), receiving anticoagulant therapy, suffering from a bleeding disorder (e.g., one that would contraindicate intramuscular injection), a prior history of heart disease, an abnormal screening troponin I laboratory value, probably or possible myocarditis or pericarditis (e.g., a subject having a 12-lead ECG that shows an average QTcF interval >450 msec, complete left bundle branch block, signs of an acute or indeterminate-age myocardial infarction, ST-T interval changes suggestive of myocardial ischemia, second- or third-degree AV block, and/or serious bradyarrhythmias or tachyarrhythmias) or pregnancy and/or breastfeeding/lactation. In some embodiments, an RNA (e.g., mRNA) composition as provided herein is administered to a subject not having received another COVID-19 vaccine and/or another influenza vaccine. In some embodiments, an RNA (e.g., mRNA) composition as provided herein is administered to a subject who has not had an allergic reaction to any component of the RNA (e.g., mRNA) composition. Examples of such allergic reaction may include, but are not limited to difficulty breathing, swelling of fact and/or throat, fast heartbeat, rash, dizziness and/or weakness. In some embodiments, an RNA (e.g., mRNA) composition as provided herein is administered to a subject who received a first dose and did not have an allergic reaction (e.g., as described herein) to the first dose.

In some embodiments where allergic reaction occurs in subject(s) after receiving a dose of an RNA (e.g., mRNA) composition as provided herein, such subject(s) may be administered one or more interventions such as treatment to manage and/or reduce symptom(s) of such allergic reactions, for example, fever-reducing and/or anti-inflammatory agents.

In some embodiments, a subject who has received at least one dose of an RNA (e.g., mRNA) composition as provided herein is informed of avoiding being exposed to a coronavirus (e.g., SARS-CoV-2) and/or an influenza virus unless and until several days (e.g., at least 7 days, at least 8 days, 9 days, at least 10 days, at least 11 days, at least 12 days, at least 13 days, at least 14 days, etc.) have passed since administration of a second dose. For example, a subject who has received at least one dose of an RNA (e.g., mRNA) composition as provided herein is informed of taking precautionary measures against SARS-CoV-2 infection and/or influenza virus infection (e.g., remaining socially distant, wearing masks, frequent hand-washing, etc.) unless and until several days (e.g., at least 7 days, at least 8 days, 9 days, at least 10 days, at least 11 days, at least 12 days, at least 13 days, at least 14 days, etc.) have passed since administration of a second dose. Accordingly, in some embodiments, methods of administering an RNA (e.g., mRNA) composition as provided herein comprise administering a second dose of such an RNA (e.g., mRNA) composition as provided herein to a subject who received a first dose and took precautionary measures to avoid being exposed to a coronavirus (e.g., SARS-CoV-2) and/or an influenza virus.

In some embodiments, mRNA compositions described herein may be delivered to a draining lymph node of a subject in need thereof, for example, for vaccine priming. In some embodiments, such delivery may be performed by intramuscular administration of a provided mRNA composition.

In some embodiments, different particular mRNA compositions may be administered to different subject population(s); alternatively or additionally, in some embodiments, different dosing regimens may be administered to different subject populations. For example, in some embodiments, mRNA compositions administered to particular subject population(s) may be characterized by one or more particular effects (e.g., incidence and/or degree of effect) in those subject populations. In some embodiments, such effect(s) may be or comprise, for example titer and/or persistence of neutralizing antibodies and/or T cells (e.g., T_{H}1-type T cells such as CD4⁺ and/or CD8⁺ T cells), protection against challenge (e.g., via injection and/or nasal exposure, etc), incidence, severity, and/or persistence of side effects (e.g., reactogenicity), etc.

In some embodiments, one or more mRNA compositions described herein may be administered according to a regimen established to reduce COVID-19 incidence and/or influenza incidence per 1000 person-years, e.g., based on a laboratory test such as nucleic acid amplification test (NAAT). In some embodiments, one or more mRNA compositions described herein may be administered according to a regimen established to reduce COVID-19 and/or influenza incidence per 1000 person-years based on a laboratory test such as nucleic acid amplification test (NAAT) in subjects receiving at least one dose of a provided mRNA composition with no serological or virological evidence (e.g., up to 7 days after receipt of the last dose) of past SARS-CoV-2 and/or influenza virus infection. In some embodiments, one or more mRNA compositions described herein may be administered according to a regimen established to reduce confirmed severe COVID-19 and/or influenza incidence per 1000 person-years. In some embodiments, one or more mRNA compositions described herein may be administered according to a regimen established to reduce confirmed severe COVID-19 and/or influenza incidence per 1000 person-years in subjects receiving at least one dose of a provided mRNA composition with no serological or virological evidence of past SARS-CoV-2 and/or past influenza virus infection.

In some embodiments, one or more mRNA compositions described herein may be administered according to a regimen established to produce neutralizing antibodies directed to a SARS-CoV-2 spike polypeptide, an influenza virus HA polypeptide, and/or immunogenic fragments thereof (e.g., RBD) as measured in serum from a subject that achieves or exceeds a reference level (e.g., a reference level determined based on human SARS-CoV-2 infection/COVID-19 convalescent sera and/or human influenza convalescent sera) for a period of time and/or induction of cell-mediated immune response (e.g., a T cell response against SARS-CoV-2 and/or influenza virus), including, e.g., in some embodiments induction of T cells that recognize at least one or more MHC-restricted (e.g., MHC class I-restricted) epitopes within a SARS-CoV-2 spike polypeptide, an influenza virus HA polypeptide, and/or immunogenic fragments thereof (e.g., RBD) for a period of time. In some such embodiments, the period of time may be at least 2 months, 3 months, at least 4 months, at least 5 months, at least 6 months, at least 7 months, at least 8 months, at least 9 months, at least 10 months, at least 11 months, at least 12 months or longer. In some embodiments, one or more epitopes recognized by vaccine-induced T cells (e.g., CD8+ T cells) may be presented on a MHC class I allele that is present in at least 50% of subjects in a population, including, e.g., at least 60%, at least 70%, at least 80%, at least 90%, or more; in some such embodiments, the MHC class I allele may be HLA-B*0702, HLA-A*2402, HLA-B*3501, HLA-B*4401, or HLA-A*0201. In some embodiments, an epitope may comprise HLA-A*0201 YLQPRTFLL (SEQ ID NO: 35); HLA-A*0201 RLQSLQTYV (SEQ ID NO: 36); HLA-A*2402 QYIKWPWYI (SEQ ID NO: 37); HLA-A*2402 NYNYLYRLF (SEQ ID NO: 38); HLA-A*2402 KWPWYIWLGF (SEQ ID NO: 39); HLA-B*3501 QPTESIVRF (SEQ ID NO: 40); HLA-B*3501 IPFAMQMAY (SEQ ID NO: 41); or HLA-B*3501 LPFNDGVYF (SEQ ID NO: 42).

In some embodiments, efficacy is assessed as COVID-19 and/or influenza incidence per 1000 person-years in individuals without serological or virological evidence of past SARS-CoV-2 infection and/or past influenza virus infection before and during vaccination regimen; alternatively or additionally, in some embodiments, efficacy is assessed as COVID-19 and/or influenza incidence per 1000 person-years in subjects with and without evidence of past SARS-CoV-2 infection and/or influenza virus infection before and during vaccination regimen. In some such embodiments, such incidence is of COVID-19 and/or influenza cases confirmed within a specific time period after the final vaccination dose (e.g., a first dose in a single-dose regimen; a second dose in a two-dose regimen, etc); in some embodiments, such time period may be within (i.e., up to and including 7 days) a particular number of days (e.g., 3, 4, 5, 6, 7, 8, 9, 10, 11, 12, 13, 14, 15, 16, 17, 18, 19, 20, 21, 22, 23, 24, 25, 26, 27, 28, 29, 30 days or more). In some embodiments, such time period may be within 7 days or within 14 days or within 21 days or within 28 days. In some embodiments, such time period may be within 7 days. In some embodiments, such time period may be within 14 days.

In some embodiments (e.g., in some embodiments of assessing efficacy), a subject is determined to have experienced COVID-19 infection or influenza virus infection if one or more of the following is established: detection of SARS-CoV-2 nucleic acid or influenza virus nucleic acid in a sample from the subject, detection of antibodies that specifically recognize SARS-CoV-2 or influenza virus (e.g., a SARS-Co-V-2 spike protein or HA polypeptide), one or more symptoms of COVID-19 infection or influenza virus infection, and combinations thereof. In some such embodiments, detection of SARS-CoV-2 or influenza virus nucleic acid may involve, for example, NAAT testing on a mid-turbinatae swap sample. In some such embodiments, detection of relevant antibodies may involve serological testing of a blood sample or portion thereof. In some such embodiments, symptoms of COVID-19 infection may be or include: fever, new or increased cough, new or increased shortness of breath, chills, new or increased muscle pain, new loss of taste or smell, sore throat, diarrhea, vomiting and combinations thereof. In some such embodiments, symptoms of COVID-19 infection may be or include: fever, new or increased cough, new or increased shortness of breath, chills, new or increased muscle pain, new loss of taste or smell, sore throat, diarrhea, vomiting, fatigue, headache, nasal congestion or runny nose, nausea, and combinations thereof. In some such embodiments, a subject is determined to have experienced COVID-19 infection if such subject both has experienced one such symptom and also has received a positive test for SARS-CoV-2 nucleic acid or antibodies, or both. In some such embodiments, a subject is determined to have experienced COVID-19 infection if such subject both has experienced one such symptom and also has received a positive test for SARS-CoV-2 nucleic acid. In some such embodiments, a subject is determined to have experienced COVID-19 infection if such subject both has experienced one such symptom and also has received a positive test for SARS-CoV-2 antibodies.

In some embodiments (e.g., in some embodiments of assessing efficacy), a subject is determined to have experienced severe COVID-19 infection if such subject has experienced one or more of: clinical signs at rest indicative or severe systemic illness (e.g., one or more of respiratory rate at greater than or equal to 30 breaths per minute, heart rate at or above 125 beats per minute, SpO₂ less than or equal to 93% on room air at sea level or a PaO₂/FiO₂ below 300 m Hg), respiratory failure (e.g., one or more of needing high-flow oxygen, noninvasive ventilation, mechanical ventilation, ECMO), evidence of shock (systolic blood pressure below 90 mm Hg, diastolic blood pressure below 60 mm Hg, requiring vasopressors), significant acute renal, hepatic, or neurologic dysfunction, admission to an intensive care unit, death, and combinations thereof.

In some embodiments, one or more mRNA compositions described herein may be administered according to a regimen established to reduce the percentage of subjects reporting at least one of the following: (i) one or more local reactions (e.g., as described herein) for up to 7 days following each dose; (ii) one or more systemic events for up to 7 days following each dose; (iii) adverse events (e.g., as described herein) from a first dose to 1 month after the last dose; and/or (iv) serious adverse events (e.g., as described herein) from a first dose to 6 months after the last dose.

In some embodiments, one or more subjects who have received an RNA (e.g., mRNA) composition as described herein may be monitored (e.g., for a period of at least 1, 2, 3, 4, 5, 6, 7, 8, 9, 10 days or more, including, for example, 1, 2, 3, 4, 5, 6, 7, 8, 9, 10, 11, 12 weeks or more, including for example 1, 2, 3, 4, 5, 6, 7, 8, 9, 10, 11, 12, 13, 14, 15, 16, 17, 18, 19, 20, 21, 22, 23, 24 months or more, including for example 1, 2, 3, 4, 5, 6, 7, 8, 9, 10 years or more) to assess, for example, presence of an immune response to component(s) of the administered composition, evidence of exposure to and/or immune response to SARS-CoV-2 or another coronavirus, evidence of any adverse event, etc. In some embodiments, monitoring may be via tele-visit. Alternatively or additionally, in some embodiments, monitoring may be in-person.

In some embodiments, a treatment effect conferred by one or more mRNA compositions described herein may be characterized by (i) a SARS-CoV-2 anti-S1 binding antibody level above a pre-determined threshold; (ii) a SARS-CoV-2 anti-RBD binding antibody level above a pre-determined threshold; (iii) a SARS-CoV-2 serum neutralizing titer above a threshold level, (iv) an anti-HA binding antibody level above a pre-determined threshold, and/or (v) an influenza virus serum neutralizing titer above a threshold, e.g., at baseline, 1 month, 3 months, 6 months, 9 months, 12 months, 18 months, and/or 24 months after completion of vaccination. In some embodiments, anti-S1 binding antibody and/or anti-RBD binding antibody levels and/or anti-HA binding antibody levels and/or serum neutralizing titers may be characterized by geometric mean concentration (GMC), geometric mean titer (GMT), or geometric mean fold-rise (GMFR).

In some embodiments, a treatment effect conferred by one or more mRNA compositions described herein may be characterized in that percentage of treated subjects showing a SARS-CoV-2 serum neutralizing titer and/or an influenza virus neutralizing titer above a pre-determined threshold, e.g., at baseline, 1 month, 3 months, 6 months, 9 months, 12 months, 18 months, and/or 24 months after completion of vaccination, is higher than the percentage of non-treated subjects showing a SARS-CoV-2 serum neutralizing titer and/or influenza virus neutralizing titer above such a pre-determined threshold (e.g., as described herein). In some embodiments, a serum neutralizing titer may be characterized by geometric mean concentration (GMC), geometric mean titer (GMT), or geometric mean fold-rise (GMFR).

In some embodiments, a treatment effect conferred by one or more mRNA compositions described herein may be characterized by detection of SARS-CoV-2 NVA-specific binding antibody.

In some embodiments, a treatment effect conferred by one or more mRNA compositions described herein may be characterized by SARS-CoV-2 detection and/or influenza virus detection by nucleic acid amplification test.

In some embodiments, a treatment effect conferred by one or more mRNA compositions described herein may be characterized by induction of cell-mediated immune response (e.g., a T cell response against SARS-CoV-2 and/or influenza virus), including, e.g., in some embodiments induction of T cells that recognize at least one or more MHC-restricted (e.g., MHC class I-restricted) epitopes within a SARS-CoV-2 spike polypeptide, HA polypeptide, and/or immunogenic fragments thereof (e.g., RBD). In some embodiments, one or more epitopes recognized by vaccine-induced T cells (e.g., CD8+ T cells) may be presented on a MHC class I allele that is present in at least 50% of subjects in a population, including, e.g., at least 60%, at least 70%, at least 80%, at least 90%, or more; in some such embodiments, the MHC class I allele may be HLA-B*0702, HLA-A*2402, HLA-B*3501, HLA-B*4401, or HLA-A*0201. In some embodiments, an epitope may comprise HLA-A*0201 YLQPRTFLL (SEQ ID NO: 35); HLA-A*0201 RLQSLQTYV (SEQ ID NO: 36); HLA-A*2402 QYIKWPWYI (SEQ ID NO: 37); HLA-A*2402 NYNYLYRLF (SEQ ID NO: 38); HLA-A*2402 KWPWYIWLGF (SEQ ID NO: 39); HLA-B*3501 QPTESIVRF (SEQ ID NO: 40); HLA-B*3501 IPFAMQMAY (SEQ ID NO: 41); or HLA-B*3501 LPFNDGVYF (SEQ ID NO: 42).

In some embodiments, primary vaccine efficacy (VE) of one or more mRNA compositions described herein may be established when there is sufficient evidence (posterior probability) that either primary VE1 or both primary VE1 and primary VE2 are >30% or higher (including, e.g., greater than 40%, greater than 50%, greater than 60%, greater than 70%, greater than 80%, greater than 90%, greater than 95%, greater than 96%, greater than 97%, greater than 98%, or higher), wherein primary VE is defined as primary VE=100×(1−IRR); and IRR is calculated as the ratio of COVID-19 and/or influenza illness rates in the vaccine group to the corresponding illness rate in the placebo group. Primary VE1 represents VE for prophylactic mRNA compositions described herein against confirmed COVID-19 and/or influenza in participants without evidence of infection before vaccination, and primary VE2 represents VE for prophylactic mRNA compositions described herein against confirmed COVID-19 and/or influenza in all participants after vaccination. In some embodiments, primary VE1 and VE2 can be evaluated sequentially to control the overall type I error of 2.5% (hierarchical testing). In some embodiments where one or more RNA (e.g., mRNA) compositions described herein are demonstrated to achieve primary VE endpoints as discussed above, secondary VE endpoints (e.g., confirmed severe COVID-19 and/or confirmed severe influenza in participants without evidence of infection before vaccination and confirmed severe COVID-19 and/or severe influenza in all participants) can be evaluated sequentially, e.g., by the same method used for the primary VE endpoint evaluation (hierarchical testing) as discussed above. In some embodiments, evaluation of primary and/or secondary VE endpoints may be based on at least 20,000 or more subjects (e.g., at least 25,000 or more subjects) randomized in a 1:1 ratio to the vaccine or placebo group, e.g., based on the following assumptions: (i) 1.0% illness rate per year in the placebo group, and (ii) 20% of the participants being non-evaluable or having serological evidence of prior infection with SARS-CoV-2 and/or influenza, potentially making them immune to further infection.

In some embodiments, one or more mRNA compositions described herein may be administered according to a regimen established to achieve maintenance and/or continued enhancement of an immune response. For example, in some embodiments, an administration regimen may include a first dose optionally followed by one or more subsequent doses; in some embodiments, need for, timing of, and/or magnitude of any such subsequent dose(s) may be selected to maintain, enhance, and/or modify one or more immune responses or features thereof. In some embodiments, number, timing, and/or amount(s) of dose(s) have been established to be effective when administered to a relevant population. In some embodiments, number, timing and/or amount(s) of dose(s) may be adjusted for an individual subject; for example, in some embodiments, one or more features of an immune response in an individual subject may be assessed at least once (and optionally more than once, for example multiple times, typically spaced apart, often at pre-selected intervals) after receipt of a first dose. For example, presence of antibodies, B cells, and/or T cells (e.g., CD4⁺ and/or CD8⁺ T cells), and/or of cytokines secreted thereby and/or identity of and/or extent of responses to particular antigen(s) and/or epitope(s) may be assessed. In some embodiments, need for, timing of, and/or amount of a subsequent dose may be determined in light of such assessments.

As noted hereinabove, in some embodiments, one or more subjects who have received an RNA (e.g., mRNA) composition as described herein may be monitored (e.g., for a period of at least 1, 2, 3, 4, 5, 6, 7, 8, 9, 10 days or more, including, for example, 1, 2, 3, 4, 5, 6, 7, 8, 9, 10, 11, 12 weeks or more, including for example 1, 2, 3, 4, 5, 6, 7, 8, 9, 10, 11, 12, 13, 14, 15, 16, 17, 18, 19, 20, 21, 22, 23, 24 months or more, including for example 1, 2, 3, 4, 5, 6, 7, 8, 9, 10 years or more) from receipt of any particular dose to assess, for example, presence of an immune response to component(s) of the administered composition, evidence of exposure to and/or immune response to SARS-CoV-2 or another coronavirus, or an influenza virus, evidence of any adverse event, etc, including to perform assessment of one or more of presence of antibodies, B cells, and/or T cells (e.g., CD4⁺ and/or CD8⁺ T cells), and/or of cytokines secreted thereby and/or identity of and/or extent of responses to particular antigen(s) and/or epitope(s) may be assessed. Administration of a composition as described herein may be in accordance with a regimen that includes one or more such monitoring steps.

For example, in some embodiments, need for, timing of, and/or amount of a second dose relative to a first dose (and/or of a subsequent dose relative to a prior dose) is assessed, determined, and/or selected such that administration of such second (or subsequent) dose achieves amplification or modification of an immune response (e.g., as described herein) observed after the first (or other prior) dose. In some embodiments, such amplification of an immune response (e.g., ones described herein) may be at least 30%, at least 40%, at least 50%, at least 60%, at least 70%, at least 80%, at least 90%, at least 95%, or higher, as compared to the level of an immune response observed after the first dose. In some embodiments, such amplification of an immune response may be at least 1.5 fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, or higher, as compared to the level of an immune response observed after the first dose.

In some embodiments, need for, timing of, and/or amount of a second (or subsequent) dose relative to a first (or other prior) dose is assessed, determined, and/or selected such that administration of the later dose extends the durability of an immune response (e.g., as described herein) observed after the earlier dose; in some such embodiments, the durability may be extended by at least 1 week, at least 2 weeks, at least 3 weeks, at least 1 month, at least 2 months, at least 3 months, at least 4 months, at least 5 months, at least 6 months, at least 7 months, at least 8 months, at least 9 months, or longer. In some embodiments, an immune response observed after the first dose may be characterized by production of neutralizing antibodies directed to a SARS-CoV-2 spike polypeptide, an influenza virus HA polypeptide, and/or immunogenic fragments thereof (e.g., RBD) as measured in serum from a subject and/or induction of cell-mediated immune response (e.g., a T cell response against SARS-CoV-2 and or an influenza virus), including, e.g., in some embodiments induction of T cells that recognize at least one or more MHC-restricted (e.g., MHC class I-restricted) epitopes within a SARS-CoV-2 spike polypeptide, an influenza virus HA polypeptide and/or immunogenic fragments thereof (e.g., RBD). In some embodiments, one or more epitopes recognized by vaccine-induced T cells (e.g., CD8+ T cells) may be presented on a MHC class I allele that is present in at least 50% of subjects in a population, including, e.g., at least 60%, at least 70%, at least 80%, at least 90%, or more; in some such embodiments, the MHC class I allele may be HLA-B*0702, HLA-A*2402, HLA-B*3501, HLA-B*4401, or HLA-A*0201. In some embodiments, an epitope may comprise HLA-A*0201 YLQPRTFLL (SEQ ID NO: 35); HLA-A*0201 RLQSLQTYV (SEQ ID NO: 36); HLA-A*2402 QYIKWPWYI (SEQ ID NO: 37); HLA-A*2402 NYNYLYRLF (SEQ ID NO: 38); HLA-A*2402 KWPWYIWLGF (SEQ ID NO: 39); HLA-B*3501 QPTESIVRF (SEQ ID NO: 40); HLA-B*3501 IPFAMQMAY (SEQ ID NO: 41); or HLA-B*3501 LPFNDGVYF (SEQ ID NO: 42).

In some embodiments, need for, timing of, and/or amount of a second dose relative to a first dose (or other subsequent dose relative to a prior dose) is assessed, determined and/or selected such that administration of such second (or subsequent) dose maintains or exceeds a reference level of an immune response; in some such embodiments, the reference level is determined based on human SARS-CoV-2 infection/COVID-19 convalescent sera, influenza convalescent sera, and/or PBMC samples drawn from subjects (e.g., at least a period of time such as at least 14 days or longer, including, e.g., 15 days, 16 days, 17 days, 18 days, 19 days, 20 days, 25 days, 30 days, 35 days, 40 days, 45 days, 50 days, 55 days, 60 days, or longer, after PCR-confirmed diagnosis when the subjects were asymptomatic. In some embodiments, an immune response may be characterized by production of neutralizing antibodies directed to a SARS-CoV-2 spike polypeptide, an influenza virus polypeptide, and/or immunogenic fragments thereof (e.g., RBD) as measured in serum from a subject and/or induction of cell-mediated immune response (e.g., a T cell response against SARS-CoV-2 and/or influenza virus), including, e.g., in some embodiments induction of T cells that recognize at least one or more MHC-restricted (e.g., MHC class I-restricted) epitopes within a SARS-CoV-2 spike polypeptide, an influenza virus polypeptide, and/or immunogenic fragments thereof (e.g., RBD). In some embodiments, one or more epitopes recognized by vaccine-induced T cells (e.g., CD8+ T cells) may be presented on a MHC class I allele that is present in at least 50% of subjects in a population, including, e.g., at least 60%, at least 70%, at least 80%, at least 90%, or more; in some such embodiments, the MHC class I allele may be HLA-B*0702, HLA-A*2402, HLA-B*3501, HLA-B*4401, or HLA-A*0201. In some embodiments, an epitope may comprise HLA-A*0201 YLQPRTFLL (SEQ ID NO: 35); HLA-A*0201 RLQSLQTYV (SEQ ID NO: 36); HLA-A*2402 QYIKWPWYI (SEQ ID NO: 37); HLA-A*2402 NYNYLYRLF (SEQ ID NO: 38); HLA-A*2402 KWPWYIWLGF (SEQ ID NO: 39); HLA-B*3501 QPTESIVRF (SEQ ID NO: 40); HLA-B*3501 IPFAMQMAY (SEQ ID NO: 41); or HLA-B*3501 LPFNDGVYF (SEQ ID NO: 42).

In some embodiments, determination of need for, timing of, and/or amount of a second (or subsequent) dose may include one or more steps of assessing, after (e.g., 7, 8, 9, 10, 11, 12, 13, 14, 15, 16, 17, 18, 19, 20, 21 days or longer after) a first (or other prior) dose, presence and/or expression levels of neutralizing antibodies directed to a SARS-CoV-2 spike polypeptide, an influenza HA polypeptide and/or immunogenic fragments thereof (e.g., RBD) as measured in serum from a subject and/or induction of cell-mediated immune response (e.g., a T cell response against SARS-CoV-2 and/or influenza virus), including, e.g., in some embodiments induction of T cells that recognize at least one or more MHC-restricted (e.g., MHC class I-restricted) epitopes within a SARS-CoV-2 spike polypeptide, an influenza virus HA polypeptide, and/or immunogenic fragments thereof (e.g., RBD). In some embodiments, one or more epitopes recognized by vaccine-induced T cells (e.g., CD8+ T cells) may be presented on a MHC class I allele that is present in at least 50% of subjects in a population, including, e.g., at least 60%, at least 70%, at least 80%, at least 90%, or more; in some such embodiments, the MHC class I allele may be HLA-B*0702, HLA-A*2402, HLA-B*3501, HLA-B*4401, or HLA-A*0201. In some embodiments, an epitope may comprise HLA-A*0201 YLQPRTFLL (SEQ ID NO: 35); HLA-A*0201 RLQSLQTYV (SEQ ID NO: 36); HLA-A*2402 QYIKWPWYI (SEQ ID NO: 37); HLA-A*2402 NYNYLYRLF (SEQ ID NO: 38); HLA-A*2402 KWPWYIWLGF (SEQ ID NO: 39); HLA-B*3501 QPTESIVRF (SEQ ID NO: 40); HLA-B*3501 IPFAMQMAY (SEQ ID NO: 41); or HLA-B*3501 LPFNDGVYF (SEQ ID NO: 42).

In some embodiments, a kit as provided herein may comprise a real-time monitoring logging device, which, for example in some embodiments, is capable of providing shipment temperatures, shipment time and/or location.

In some embodiments, an RNA (e.g., mRNA) composition as described herein may be shipped, stored, and/or utilized, in a container (such as a vial or syringe), e.g., a glass container (such as a glass vial or syringe), which, in some embodiments, may be a single-dose container or a multi-dose container (e.g., may be arranged and constructed to hold, and/or in some embodiments may hold, a single dose, or multiple doses of a product for administration). In some embodiments, a multi-dose container (such as a multi-dose vial or syringe) may be arranged and constructed to hold, and/or may hold 2, 3, 4, 5, 6, 7, 8, 9, 10 or more doses; in some particular embodiments, it may be designed to hold and/or may hold 5 doses. In some embodiments, a single-dose or multi-dose container (such as a single-dose or multi-dose vial or syringe) may be arranged and constructed to hold and/or may hold a volume or amount greater than the indicated number of doses, e.g., in order to permit some loss in transfer and/or administration. In some embodiments, an RNA (e.g., mRNA) composition as described herein may be shipped, stored, and/or utilized, in a preservative-free glass container (e.g., a preservative-free glass vial or syringe, e.g., a single-dose or multi-dose preservative-free glass vial or syringe). In some embodiments, an RNA (e.g., mRNA) composition as described herein may be shipped, stored, and/or utilized, in a preservative-free glass container (e.g., a preservative-free glass vial or syringe, e.g., a single-dose or multi-dose preservative-free glass vial or syringe) that contains a frozen liquid, e.g., in some embodiments 0.45 ml of frozen liquid (e.g., including 5 doses). In some embodiments, an RNA (e.g., mRNA) composition as described herein and/or a container (e.g., a vial or syringe) in which it is disposed, is shipped, stored, and/or utilized may be maintained at a temperature below room temperature, at or below 4° C., at or below 0° C., at or below −20° C., at or below −60° C., at or below −70° C., at or below −80° C., at or below −90° C., etc. In some embodiments, an RNA (e.g., mRNA) composition as described herein and/or a container (e.g., a viral or syringe) in which it is disposed, is shipped, stored, and/or utilized may be maintained at a temperature between −80° C. and −60° C. and in some embodiments protected from light. In some embodiments, an RNA (e.g., mRNA) composition as described herein and/or a container (e.g., a viral or syringe) in which it is disposed, is shipped, stored, and/or utilized may be maintained at a temperature below about 25° C., and in some embodiments protected from light. In some embodiments, an RNA (e.g., mRNA) composition as described herein and/or a container (e.g., a viral or syringe) in which it is disposed, is shipped, stored, and/or utilized may be maintained at a temperature below about 5° C. (e.g., below about 4° C.), and in some embodiments protected from light. In some embodiments, an RNA (e.g., mRNA) composition as described herein and/or a container (e.g., a viral or syringe) in which it is disposed, is shipped, stored, and/or utilized may be maintained at a temperature below about −20° C., and in some embodiments protected from light. In some embodiments, an RNA (e.g., mRNA) composition as described herein and/or a container (e.g., a viral or syringe) in which it is disposed, is shipped, stored, and/or utilized may be maintained at a temperature above about −60° C. (e.g., in some embodiments at or above about −20° C., and in some embodiments at or above about 4-5° C., in either case optionally below about 25° C.), and in some embodiments protected from light, or otherwise without affirmative steps (e.g., cooling measures) taken to achieve a storage temperature materially below about −20° C.

In some embodiments, an RNA (e.g., mRNA) composition as described herein and/or a container (e.g., a vial or syringe) in which it is disposed is shipped, stored, and/or utilized together with and/or in the context of a thermally protective material or container and/or of a temperature adjusting material. For example, in some embodiments, an RNA (e.g., mRNA) composition as described herein and/or a container (e.g., a vial or syringe) in which it is disposed is shipped, stored, and/or utilized together with ice and/or dry ice and/or with an insulating material. In some particular embodiments, a container (e.g., a vial or syringe) in which an RNA (e.g., mRNA) composition is disposed is positioned in a tray or other retaining device and is further contacted with (or otherwise in the presence of) temperature adjusting (e.g., ice and/or dry ice) material and/or insulating material. In some embodiments, multiple containers (e.g., multiple vials or syringes such as single use or multi-use vials or syringes as described herein) in which a provided RNA (e.g., mRNA) composition is disposed are co-localized (e.g., in a common tray, rack, box, etc.) and packaged with (or otherwise in the presence of) temperature adjusting (e.g., ice and/or dry ice) material and/or insulating material. To give but one example, in some embodiments, multiple containers (e.g., multiple vials or syringes such as single use or multi-use vials or syringes as described herein) in which an RNA (e.g., mRNA) composition is disposed are positioned in a common tray or rack, and multiple such trays or racks are stacked in a carton that is surrounded by a temperature adjusting material (e.g., dry ice) in a thermal (e.g., insulated) shipper. In some embodiments, temperature adjusting material is replenished periodically (e.g., within 24 hours of arrival at a site, and/or every 2 hours, 4 hours, 6 hours, 8 hours, 10 hours, 12 hours, 14 hours, 16 hours, 18 hours, 20 hours, 22 hours, 1 day, 2 days, 3 days, 4 days, 5 days, 6 days, 7 days, 8 days, 9 days, 10 days, etc.). Preferably, re-entry into a thermal shipper should be infrequent, and desirably should not occur more than twice a day. In some embodiments, a thermal shipper is re-closed within 5, 4, 3, 2, or 1 minute, or less, of having been opened. In some embodiments, a provided RNA (e.g., mRNA) composition that has been stored within a thermal shipper for a period of time, optionally within a particular temperature range remains useful. For example, in some embodiments, if a thermal shipper as described herein containing a provided RNA (e.g., mRNA) composition is or has been maintained (e.g., stored) at a temperature within a range of about 15° C. to about 25° C., the RNA (e.g., mRNA) composition may be used for up to 10 days; that is, in some embodiments, a provided RNA (e.g., mRNA) composition that has been maintained within a thermal shipper, which thermal shipper is at a temperature within a range of about 15° C. to about 25° C., for a period of not more than 10 days is administered to a subject. Alternatively or additionally, in some embodiments, if a provided RNA (e.g., mRNA) composition is or has been maintained (e.g., stored) within a thermal shipper, which thermal shipper has been maintained (e.g., stored) at a temperature within a range of about 15° C. to about 25° C., it may be used for up to 10 days; that is, in some embodiments, a provided RNA (e.g., mRNA) composition that has been maintained within a thermal shipper, which thermal shipper has been maintained at a temperature within a range of about 15° C. to about 25° C. for a period of not more than 10 days is administered to a subject.

In some embodiments, a provided RNA (e.g., mRNA) composition is shipped and/or stored in a frozen state. In some embodiments, a provided RNA (e.g., mRNA composition is shipped and/or stored as a frozen suspension, which in some embodiments does not contain preservative. In some embodiments, a frozen RNA (e.g., mRNA) composition is thawed. In some embodiments, a thawed RNA (e.g., mRNA) composition (e.g., a suspension) may contain white to off-white opaque amorphous particles. In some embodiments, a thawed RNA (e.g., mRNA) composition may be used for up to a small number (e.g., 1, 2, 3, 4, 5, or 6) of days after thawing if maintained (e.g., stored) at a temperature at or below room temperature (e.g., below about 30° C., 25° C., 20° C., 15° C., 10° C., 8° C., 4° C., etc). In some embodiments, a thawed RNA (e.g., mRNA) composition may be used after being stored (e.g., for such small number of days) at a temperature between about 2° C. and about 8° C.; alternatively or additionally, a thawed RNA (e.g., mRNA) composition may be used within a small number (e.g., 1, 2, 3, 4, 5, 6) of hours after thawing at room temperature. Thus, in some embodiments, a provided RNA (e.g., mRNA) composition that has been thawed and maintained at a temperature at or below room temperature, and in some embodiments between about 2° C. and about 8° C., for not more than 6, 5, 4, 3, 2, or 1 days is administered to a subject. Alternatively or additionally, in some embodiments, a provided RNA (e.g., mRNA) composition that has been thawed and maintained at room temperature for not more than 6, 5, 4, 3, 2, or 1 hours is administered to a subject. In some embodiments, a provided RNA (e.g., mRNA) composition is shipped and/or stored in a concentrated state. In some embodiments, such a concentrated composition is diluted prior to administration. In some embodiments, a diluted composition is administered within a period of about 10, 9, 8, 7, 6, 5, 4, 3, 2, or 1 hour(s) post-dilution; in some embodiments, such administration is within 6 hours post-dilution. Thus, in some embodiments, diluted preparation of a provided RNA (e.g., mRNA) composition is administered to a subject within 6 hours post-dilution (e.g., as described herein after having been maintained at an appropriate temperature, e.g., at a temperature below room temperature, at or below 4° C., at or below 0° C., at or below −20° C., at or below −60° C., at or below −70° C., at or below −80° C., etc, and typically at or above about 2° C., for example between about 2° C. and about 8° C. or between about 2° C. and about 25° C.). In some embodiments, unused composition is discarded within several hours (e.g., about 10, about 9, about 8, about 7, about 6, about 5 or fewer hours) after dilution; in some embodiments, unused composition is discarded within 6 hours of dilution.

In some embodiments, an RNA (e.g., mRNA) composition that is stored, shipped or utilized (e.g., a frozen composition, a liquid concentrated composition, a diluted liquid composition, etc.) may have been maintained at a temperature materially above −60° C. for a period of time of at least 1, 2, 3, 4, 5, 6, 7 days or more, or at least 1, 2, 3, 4, 5, 6, 7, 8, 9, 10 weeks or more, or at least 1, 2, 3, 4, 5, 6, 7, 8, 9, 10, 11, 12 months or more; in some such embodiments, such composition may have been maintained at a temperature at or above about −20° C. for such period of time, and/or at a temperature up to or about 4-5° C. for such period of time, and/or may have been maintained at a temperature above about 4-5° C., and optionally about 25° C. for a period of time up that is less than two (2) months and/or optionally up to about one (1) month. In some embodiments, such composition may not have been stored, shipped or utilized (or otherwise exposed to) a temperature materially above about 4-5° C., and in particular not at or near a temperature of about 25° C. for a period of time as long as about 2 weeks, or in some embodiments 1 week. In some embodiments, such composition may not have been stored, shipped or utilized (or otherwise exposed to) a temperature materially above about −20° C., and in particular not at or near a temperature of about 4-5° C. for a period of time as long as about 12 months, 11 months, 10 months, 9 months, 8 months, 7 months, 6 months, 5 months, 4 months, 3 months, 2 months, or, in some embodiments, for a period of time as long as about 8 weeks or 6 weeks or materially more than about 2 months or, in some embodiments, 3 months or, in some embodiments 4 months.

In some embodiments, an RNA (e.g., mRNA) composition that is stored, shipped or utilized (e.g., a frozen composition, a liquid concentrated composition, a diluted liquid composition, etc.) may be protected from light. In some embodiments, one or more steps may be taken to reduce or minimize exposure to light for such compositions (e.g., which may be disposed within a container such as a vial or a syringe). In some embodiments, exposure to direct sunlight and/or to ultraviolent light is avoided. In some embodiments, a diluted solution may be handled and/or utilized under normal room light conditions (e.g., without particular steps taken to minimize or reduce exposure to room light). It should be understood that strict adherence to aseptic techniques is desirable during handling (e.g., diluting and/or administration) of an RNA (e.g., mRNA) composition as described herein. In some embodiments, an RNA (e.g., mRNA) composition as described herein is not administered (e.g., is not injected) intravenously. In some embodiments, an RNA (e.g., mRNA) composition as described herein is not administered (e.g., is not injected) intradermally. In some embodiments, an RNA (e.g., mRNA) composition as described herein is not administered (e.g., is not injected) subcutaneously. In some embodiments, an RNA (e.g., mRNA) composition as described herein is not administered (e.g., is not injected) any of intravenously, intradermally, or subcutaneously. In some embodiments, an RNA (e.g., mRNA) composition as described herein is not administered to a subject with a known hypersensitivity to any ingredient thereof. In some embodiments, a subject to whom an RNA (e.g., mRNA) composition has been administered is monitored for one or more signs of anaphylaxis. In some embodiments, an RNA (e.g., mRNA composition) as described herein is not administered (in particular, not administered via IM injection) to a subject with bleeding diathesis or a condition associated with prolonged bleeding. In some embodiments, a subject to whom an RNA (e.g., mRNA) composition is administered had previously received at least one dose of a different vaccine for SARS-CoV-2 and/or influenza; in some embodiments, a subject to whom an RNA (e.g., mRNA) composition is administered had not previously received a different vaccine for SARS-CoV-2 and/or influenza. In some embodiments, a subject's temperature is taken promptly prior to administration of an RNA (e.g., mRNA) composition (e.g., shortly before or after thawing, dilution, and/or administration of such composition); in some embodiments, if such subject is determined to be febrile, administration is delayed or canceled. In some embodiments, an RNA (e.g., mRNA) composition as described herein is not administered to a subject who is receiving anticoagulant therapy or is suffering from or susceptible to a bleeding disorder or condition that would contraindicate intramuscular injection. In some embodiments, an RNA (e.g., mRNA) composition as described herein is administered by a healthcare professional who has communicated with the subject receiving the composition information relating to side effects and risks. In some embodiments, an RNA (e.g., mRNA) composition as described herein is administered by a healthcare professional who has agreed to submit an adverse event report for any serious adverse events, which may include for example one or more of death, development of a disability or congenital anomaly/birth defect (e.g., in a child of the subject), in-patient hospitalization (including prolongation of an existing hospitalization), a life-threatening event, a medical or surgical intervention to prevent death, a persistent or significant or substantial disruption of the ability to conduct normal life functions; or another important medical event that may jeopardize the individual and may require medical or surgical intervention (treatment) to prevent one of the other outcomes.

In some embodiments, provided RNA compositions are administered to a population of individuals under 18 years of age, or under 17 years of age, or under 16 years of age, or under 15 years of age, or under 14 years of age, or under 13 years of age, for example according to a regimen established to have a rate of incidence for one or more of the local reaction events indicated below that does not exceed the rate of incidence indicated below:
pain at the injection site (75% after a first dose and/or a second dose, and/or a lower incidence after a second dose, e.g., 65% after a second dose);
redness at the injection site (less than 5% after a first dose and/or a second dose); and/or
swelling at the injection site (less than 5% after a first dose and/or a second dose).

In some embodiments, provided RNA compositions are administered to a population of individuals under 18 years of age, or under 17 years of age, or under 16 years of age, or under 15 years of age, or under 14 years of age, or under 13 years of age, for example according to a regimen established to have a rate of incidence for one or more of the systemic reaction events indicated below that does not exceed the rate of incidence indicated below:
fatigue (55% after a first dose and/or a second dose);
headache (50% after a first dose and/or a second dose);
muscle pain (40% after a first dose and/or a second dose);
chills (40% after a first dose and/or a second dose);
joint pain (20% after a first dose and/or a second dose);
fever (25% after a first dose and/or a second dose);
vomiting (10% after a first dose and/or a second dose); and/or
diarrhea (10% after a first dose and/or a second dose).

In some embodiments, medication that alleviates one or more symptoms of one or more local reaction and/or systemic reaction events (e.g., described herein) are administered to individuals under 18 years of age, or under 17 years of age, or under 16 years of age, or under 15 years of age, or under 14 years of age, or under 13 years of age who have been administered with provided RNA compositions and have experienced one or more of the local and/or systemic reaction events (e.g., described herein). In some embodiments, antipyretic and/or pain medication can be administered to such individuals.

### BRIEF DESCRIPTION OF THE DRAWINGS

FIG. 1. Schematics of an exemplary vaccination regimen.

FIG. 2. Immune Responses in Mice administered Covid/Influenza Combination Vaccines. Neutralization titers in mice are shown 21 days after administration of a first and a second dose of an influenza vaccine alone, a SARS-CoV-2 vaccine alone, or combinations thereof. Mouse groups indicated on x-axes. “Quad Flu” or “Flu” refers to mice administered a vaccine comprising four RNAs, each encoding an HA protein of an H1N1/Wisconsin, H3N2/Darwin, By/Phuket, or Bv/Austria influenza strain, at a ratio of 1:1:1:1. “COVID” or “Bivalent COVID” refers to mice administered a bivalent SARS-CoV-2 vaccine, comprising RNA encoding a SARS-CoV-2 S protein of a Wuhan strain and RNA encoding a SARS-CoV-2 S protein comprising mutations characteristic of a BA.4/5 Omicron variant, at a ratio of 1:1. Quad flu and bivalent COVID vaccines were produced by mixing RNA prior to LNP formulation (i.e., RNAs were in the same LNPs). Influenza/SARS-CoV-2 combinations were administered by (i) mixing vaccines prior to administering in a single injection (“Post-Mix”) or (ii) two injections, at separate injection sites (“Separate Injections”). “Fluad” and “Fluzone” refer to commercially available influenza vaccines. Neutralization of H1N1/Wisconsin, H3N2/Darwin, By/Phuket, and Bv/Austria influenza strains by sera collected from mice 3 weeks after administration of a first dose of a vaccine, and as determined by Microneutralization (MNT) Assay are shown in (A)-(D), respectively. Pseudovirus neutralization titers against a SARS-CoV-2 Wuhan strain and a BA.4/5 Omicron strain by sera collected from mice 3 weeks after administration of a first dose of a vaccine are shown in (E). (F) summarizes SARS-CoV-2 (“COVID vaccine responses”) and Influenza (“Flu vaccine responses”) neutralization responses in mice administered (i) 0.8 μg of Bivalent COVID alone (“Bivalent Covid”), (ii) 0.8 μg of Bivalent COVID and 0.8 μg of Quadrivalent Flu (“Bivalent COVID+Quad Flu”), or (iii) 0.8 μg of Quadrivalent Flu alone (“Quad Flu”), 3 weeks after administration of a first dose of a vaccine. Pseudovirus neutralization titers against a SARS-CoV-2 Wuhan strain and a BA.4/5 Omicron strain by sera collected from mice 2 weeks after administration of a second dose of a vaccine are shown in (G). Neutralization of H1N1/Wisconsin, H3N2/Darwin, By/Phuket, and Bv/Austria influenza strains by sera collected from mice 2 weeks after administration of a second dose of a vaccine, and as determined by MNT Assay are shown in (H)-(K), respectively. (L) summarizes SARS-CoV-2 (“Bivalent COVID vaccine responses”) and Influenza (“Quadrivalent Flu vaccine responses”) neutralization responses in mice administered (i) 0.8 μg of Bivalent COVID alone (“Bivalent Covid”), (ii) 0.8 μg of Bivalent COVID and 0.8 μg of Quadrivalent Flu (“Bivalent COVID+Quad Flu”), or (iii) 0.8 μg of Quadrivalent Flu alone (“Quad Flu”), 3 weeks after administration of a first dose of a vaccine.

FIG. 3. Exemplary method for co-administering two or more vaccines in single syringe. (A) shows an exemplary method for co-administering two vaccines, and (B) shows an exemplary method for co-administering three vaccines.

FIG. 4. BNT16b2 and RSVpreF vaccines do not interfere with one another when co-administered. Shown is a Forrest plot, indicating the Geometric Mean Ratios (GMRs) for [RSVpreF+BNT162b2] coadministered with a placebo vs each vaccine administered alone, 1 month after vaccination. GMR=geometric mean ratio, LLOQ=lower limit of quantitation, NT50=50% neutralizating titer; [RSVpreF+BNT162b2] denotes admixture of RSVpreF and bivalent BNT162b2 (Wuhan+BA.4/5 vaccines. GMRs and 2-sided Cis were calculated by exponentiating the mean differences of the logarithms of the titers (Intervention Group minus Reference Group and the corresponding Cis (based on the Student t distribution).

### DETAILED DESCRIPTION OF CERTAIN EMBODIMENTS

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

The term “about” means approximately or nearly, and in the context of a numerical value or range set forth herein in one embodiment means±20%, ±10%, ±5%, or ±3% of the numerical value or range recited or claimed. The terms “a” and “an” and “the” and similar reference used in the context of describing the present disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the present disclosure and does not pose a limitation on the scope of the claims. No language in the specification should be construed as indicating any non-claimed element essential to the practice of the present disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of” or “consisting essentially of”.

Terms such as “reduce”, “decrease”, “inhibit” or “impair” as used herein relate to an overall reduction or the ability to cause an overall reduction, preferably of at least 5%, at least 10%, at least 20%, at least 50%, at least 75% or even more, in the level. These terms include a complete or essentially complete inhibition, i.e., a reduction to zero or essentially to zero.

Terms such as “increase”, “enhance” or “exceed” preferably relate to an increase or enhancement by at least 10%, at least 20%, at least 30%, at least 40%, at least 50%, at least 80%, at least 100%, at least 200%, at least 500%, or even more.

According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 150 amino acids, but the terms “peptide”, “protein” and “polypeptide” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, antigens for vaccination and immunostimulants such as cytokines.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified. As used herein, a wild type SARS-CoV-2 S protein refers to a protein having the sequence of the S protein of the first detected Wuhan strain of SARS-CoV-2 (having SEQ ID NO: 1).

In some embodiments, the present disclosure refers to a SARS-CoV-2 variant that is prevalent and/or rapidly spreading in a relevant jurisdiction. In some embodiments, such variants may be identified based on publicly available data (e.g., data provided in the GISAID Initiative database: http_//www.gisaid.org, and/or data provided by the World Health Organization WHO (e.g., as provided at https_//www.who.int/activities/tracking-SARS-CoV-2-variants). In some embodiments, such a variant refers to a variant disclosed herein.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups: glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS: needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the present disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

As used herein, an “instructional material” or “instructions” includes a publication, a recording, a diagram, or any other medium of expression which can be used to communicate the usefulness of the compositions and methods of the present disclosure. The instructional material of the kit of the present disclosure may, for example, be affixed to a container which contains the compositions of the present disclosure or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present disclosure means “made through genetic engineering”. Preferably, a “recombinant object” such as a recombinant nucleic acid in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

“Physiological pH” as used herein refers to a pH of about 7.5.

The term “genetic modification” or simply “modification” includes the transfection of cells with nucleic acid. The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro or in vivo, e.g. the cell can form part of an organ, a tissue and/or an organism of a patient. According to the present disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding antigen is transiently transfected into cells. RNA can be transfected into cells to transiently express its coded protein.

The term “seroconversion” includes a ≥4-fold rise from before vaccination to 1-month post Dose 2.

### Coronavirus

Coronaviruses are enveloped, positive-sense, single-stranded RNA ((+) ssRNA) viruses. They have the largest genomes (26-32 kb) among known RNA viruses and are phylogenetically divided into four genera (α, β, γ, and δ), with betacoronaviruses further subdivided into four lineages (A, B, C, and D). Coronaviruses infect a wide range of avian and mammalian species, including humans. Some human coronaviruses generally cause mild respiratory diseases, although severity can be greater in infants, the elderly, and the immunocompromised. Middle East respiratory syndrome coronavirus (MERS-CoV) and severe acute respiratory syndrome coronavirus (SARS-COV), belonging to betacoronavirus lineages C and B, respectively, are highly pathogenic. Both viruses emerged into the human population from animal reservoirs within the last 15 years and caused outbreaks with high case-fatality rates. The outbreak of severe acute respiratory syndrome coronavirus-2 (SARS-CoV-2) that causes atypical pneumonia (coronavirus disease 2019; COVID-19) has raged in China since mid-December 2019, and has developed to be a public health emergency of international concern. SARS-CoV-2 (MN908947.3) belongs to betacoronavirus lineage B. It has at least 70% sequence similarity to SARS-COV.

In general, coronaviruses have four structural proteins, namely, envelope (E), membrane (M), nucleocapsid (N), and spike(S). The E and M proteins have important functions in the viral assembly, and the N protein is necessary for viral RNA synthesis. The critical glycoprotein S is responsible for virus binding and entry into target cells. The S protein is synthesized as a single-chain inactive precursor that is cleaved by furin-like host proteases in the producing cell into two noncovalently associated subunits, S1 and S2. The S1 subunit contains the receptor-binding domain (RBD), which recognizes the host-cell receptor. The S2 subunit contains the fusion peptide, two heptad repeats, and a transmembrane domain, all of which are required to mediate fusion of the viral and host-cell membranes by undergoing a large conformational rearrangement. The S1 and S2 subunits trimerize to form a large prefusion spike.

The S precursor protein of SARS-CoV-2 can be proteolytically cleaved into S1 (685 aa) and S2 (588 aa) subunits. The S1 subunit comprises the receptor-binding domain (RBD), which mediates virus entry into sensitive cells through the host angiotensin-converting enzyme 2 (ACE2) receptor.

### Influenza Viruses

Influenza illness is caused by influenza viruses, of which there are four types: A, B, C, and D. Types A and B are responsible for the seasonal epidemics that occur every winter in the United States (also known as flu season). Type A viruses are the only type to date that have caused a pandemic (i.e., a global epidemic). Type C viruses generally cause mild illness and are not thought to cause human epidemics, while type D viruses primarily affect cattle, and are not known to infect or cause illness in humans.

Influenza A viruses are divided into subtypes based on two surface proteins: hemagglutinin (HA) and neuraminidase (NA). 18 HA subtypes and 11 different NA subtypes are known to exist, and more than 130 influenza A subtype combinations have been observed, although many more subtype combinations are possible, given the virus's propensity for “reassortment” (i.e., the process in which influenza viruses swap gene segments, which can occur when two viruses infect a host at the same time). Subtypes H1N1 and H3N2 are the type A viruses that are currently common in humans. Subtypes can be further broken down into “clades” and “sub-clades” (also known as “groups” and “sub-groups”, respectively), which are organized based on HA gene sequences.

Clades and sub-clades may be genetically distinct from one another while not being antigenically distinct. For example, it may be possible for two viruses to have distinct HA gene sequences, and thus be genetically distinct, and yet still be bound and neutralized by a given antibody, and thus not antigenically distinct.

Currently circulating influenza A (H1N1) viruses are related to the 2009 H1N1 virus that emerged in the spring of 2009 and caused the flu pandemic of that year. These viruses, also called A(H1N1)pdm09 viruses or “2009 H1N1”, have continued to circulate seasonally since first being discovered, and have undergone several changes both genetically and antigenically.

Influenza A (H3N2) viruses also comprise many separate, genetically different clades in recent years that continue to circulate.

Influenza B viruses are classified by lineage rather than subtype. Two lineages of influenza B viruses exist: B/Yamagata and B/Victoria, each of which can be further divided into clades and sub-clades. Influenza B viruses generally change more slowly than influenza A viruses, both genetically and antigenically. In recent years, both B/Yamagata and B/Victoria have been in co-circulation, although the proportion from each lineage can vary depending on location and season.

Influenza virus names usually indicate type (A, B, C, D), host of origin (although for humans, the host of origin is usually not indicated), geographical origin, strain number, and year of collection. For influenza A viruses, HA and NA descriptions are provided in parenthesis.

Seasonal flu vaccines are typically formulated to provide protection against multiple influenza viruses that are known to cause epidemics. In recent years, vaccines have been formulated as tetravalent vaccines, to provide antigens against H1N1, H3N2, B/Victoria, and B/Yamagata viruses. In some embodiments, an influenza vaccine can protect both against the viruses that the vaccine comprises or delivers antigens from, and antigenically similar viruses.

### RSV Viruses

Respiratory syncytial virus (RSV), also called human respiratory syncytial virus (hRSV) and human orthopneumovirus, is a common, contagious virus that can cause respiratory tract infections.

RSV is a negative-sense, single-stranded RNA virus. RSV belongs to the genus Orthopneumovirus, family Pneumoviridae, order Mononegavirales. Its name is derived from the large cells known as syncytia that form when infected cells fuse. RSV's genome is linear and approximately 15,000 nucleotides in length. It is non-segmented, meaning that, unlike influenza, RSV cannot participate in the type of genetic reassortment and antigenic shifts responsible for large pandemics. RSV has 10 genes encoding 11 proteins. The gene order is NS1-NS2-N-P-M-SH-G-F-M2-L, with the NS1 and NS2 gene serving as nonstructural promoter genes.

RSV is a medium-sized (˜150 nm) enveloped virus. While many particles are spherical, filamentous species have also been identified. The genome rests within a helical nucleocapsid and is surrounded by matrix protein and an envelope containing viral glycoproteins.

RSV is divided into two antigenic subtypes, A and B, based on reactivity of F and G surface proteins to monoclonal antibodies. Both subtypes tend to circulate simultaneously within local epidemics, although subtype A tends to be more prevalent. Generally, RSV subtype A (RSV A) is thought to be more virulent than RSV subtype B (RSV B), with higher viral loads and faster transmission time. To date, 16 RSVA and 22 RSVB clades have been identified. Among RSVA, the GA1, GA2, GA5, and GA7 clades predominate; GA7 is found only in the United States. Among RSVB, the BA clade predominates worldwide.

RSV can cause outbreaks both in the community and in hospital settings. Following initial infection via the eyes or nose, the virus infects the epithelial cells of the upper and lower airway, causing inflammation, cell damage, and airway obstruction. A variety of methods are available for viral detection and diagnosis of RSV including antigen testing, molecular testing, and viral culture.

Respiratory syncytial virus (RSV) is a common cause of childhood illness and hospitalization in infants. It causes annual outbreaks of respiratory illnesses in all age groups. In most regions of the United States, RSV season starts in the fall and peaks in the winter, but the timing and severity of RSV season in a given community can vary from year to year.

RSV can be a particular concern in patients with respiratory illness, especially during RSV season.

RSV vaccines are available to protect older adults (e.g., subjects 60 years and older) from severe RSV (e.g., RSV vaccines described herein). Monoclonal antibody products are also available to protect infants and young children from severe RSV.

In some embodiments, an RSV vaccine is administered as a single dose.

### Clinical Description and Diagnosis

### In Infants and Young Children

RSV infection can cause a variety of respiratory illnesses and symptoms in infants and young children. It most commonly causes a cold-like illness but can also cause lower respiratory infections like bronchiolitis and pneumonia.

Two to three percent of infants with RSV infection may need to be hospitalized. Severe disease most commonly occurs in very young infants. Additionally, children with any of the following underlying conditions are considered at increased risk:
Premature infants
Infants, especially those 6 months and younger.
Children younger than 2 years old with chronic lung disease or congenital heart disease
Children with suppressed or weakened immune systems
Children who have neuromuscular disorders or a congenital anomaly, including those who have difficulty swallowing or clearing mucus secretions
Children with severe cystic fibrosis

Infants and young children with RSV infection may have rhinorrhea and a decrease in appetite before any other symptoms appear. Cough usually develops 1 to 3 days later. Soon after the cough develops, sneezing, fever, and wheezing may occur. Symptoms in very young infants can include irritability, decreased activity, and/or apnea. Most otherwise healthy infants and young children who are infected with RSV do not need hospitalization. Those who are hospitalized may require oxygen, rehydration, and/or mechanical ventilation. Most improve with supportive care and are discharged in a few days.

In Older Adults and Adults with Chronic Medical Conditions

Adults infected with RSV usually have mild or no symptoms. Symptoms are usually consistent with an upper respiratory tract infection and can include rhinorrhea, pharyngitis, cough, headache, fatigue, and fever. Disease usually lasts less than 5 days.

Some adults, however, can experience more severe symptoms consistent with a lower respiratory tract infection, such as pneumonia. Epidemiologic evidence indicates that people 60 years and older who are at highest risk of severe RSV disease include those with any of the following chronic conditions:
Lung disease (such as chronic obstructive pulmonary disease [COPD] and asthma)
Chronic cardiovascular diseases (such as congestive heart failure and coronary artery disease)
Diabetes mellitus
Neurologic conditions
Kidney disorders
Liver disorders
Hematologic disorders
Immune compromise
Other underlying conditions that a health care provider determines might increase risk of severe respiratory disease

Other underlying factors that can increase the risk of severe RSV-associated respiratory illness can include the following:
Frailty
Advanced age
Residence in a nursing home or other long-term care facility.
Other underlying factors that a health care provider determines might increase the risk for severe respiratory disease

RSV can sometimes also lead to exacerbation of serious conditions such as:
Asthma
Chronic obstructive pulmonary disease (COPD); and
Congestive heart failure.

### Clinical Laboratory Testing

Clinical symptoms of RSV are nonspecific and can overlap with other viral respiratory infections, as well as some bacterial infections. Several types of laboratory tests are available for confirming RSV infection. These tests may be performed on upper and lower respiratory specimens.

The most commonly used types of RSV clinical laboratory tests include:
Real-time reverse transcription-polymerase chain reaction (rRT-PCR), which is more sensitive than culture and antigen testing
Antigen testing, which is sensitive in children but less sensitive in adults

Less commonly used tests include:
Viral culture
Serology, which is usually only used for research and surveillance studies

Some tests can differentiate between RSV subtypes (A and B).

### For Infants and Young Children

Both rRT-PCR and antigen detection tests are effective methods for diagnosing RSV infection in infants and young children. Sensitivity of RSV antigen detection tests generally ranges from 80% to 90% in this age group.

### For Older Children, Adolescents, and Adults

Healthcare providers should use highly sensitive rRT-PCR assays when testing older children and adults for RSV. rRT-PCR assays are now commercially available for RSV. The sensitivity of these assays often exceeds the sensitivity of virus isolation and antigen detection methods. Antigen tests are not sensitive for older children and adults because they may have lower viral loads in their respiratory specimens.

### Antigen

The present disclosure, among other things, provides compositions that comprise and methods that use RNA encoding an amino acid sequence comprising a SARS-CoV-2 S protein, RNA encoding an amino acid sequence comprising an influenza virus HA protein, immunogenic variants thereof, or immunogenic fragments of the SARS-CoV-2 S protein, influenza HA protein, or immunogenic variants thereof. Thus, in some embodiments, an RNA encodes a peptide or protein comprising at least an epitope of a SARS-CoV-2 S protein, at least an epitope of an influenza HA protein, or immunogenic variants thereof for inducing an immune response against a coronavirus S protein, in particular a SARS-CoV-2 S protein, or an influenza HA protein in a subject. The amino acid sequence comprising a SARS-CoV-2 S protein, an influenza HA protein, immunogenic variants thereof, or immunogenic fragments of the SARS-CoV-2 S protein, influenza HA protein, or the immunogenic variants thereof (i.e., the antigenic peptide or protein) are examples of a “vaccine antigen”, “peptide and protein antigen”, “antigen molecule” or simply “antigen”. The SARS-CoV-2 S protein, influenza HA protein, immunogenic variants thereof, or immunogenic fragments of the SARS-CoV-2 S protein, influenza HA protein, or immunogenic variants thereof are also examples of “antigenic peptide or protein” or “antigenic sequence”.

As used herein, the SARS-CoV-2 coronavirus full length spike(S) protein of the Wuhan variant refers to a polypeptide consisting of 1273 amino acids and having an amino acid sequence according to SEQ ID NO: 1):

(SEQ ID NO: 1)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

For purposes of the present disclosure, the above sequence is considered the wildtype SARS-CoV-2 S protein amino acid sequence. Position numberings in SARS-CoV-2 S protein given herein are in relation to the amino acid sequence according to SEQ ID NO: 1 and corresponding positions in SARS-CoV-2 S protein variants.

In specific embodiments, full length spike(S) protein encoded by an RNA described herein can be modified in such a way that the prototypical prefusion conformation is stabilized. Certain mutations that stabilize a prefusion confirmation are known in the art, e.g., as disclosed in WO 2021243122 A2, WO 2023/001259 A1, US 2023/0021583 A1, and Hsieh, Ching-Lin, et al. (“Structure-based design of prefusion-stabilized SARS-CoV-2 spikes,” Science 369.6510 (2020): 1501-1505), the contents of each which are incorporated by reference herein in their entirety. In some embodiments, a SARS-CoV-2 S protein can be stabilized by introducing one or more glycine or proline mutations (e.g., one or more glycine or proline mutations in the crown of the helix turn region in the S protein, in the 12 amino acids between the heptad region 1 (HR1) and central helix (CH) or heptad region 2 (HR2) regions of the S2 subunit. In some embodiments, a SARS-CoV-2 S protein can be stabilized by introducing one or more glycine or proline mutations at one positions corresponding to one or more of L984, D985, K986, and V987 (positions relative to SEQ ID NO: 1)). In some embodiments, a Spike protein comprises glycine mutations at positions corresponding to each of L984, D985, K986, and V987 (positions relative to SEQ ID NO: 1).

In some embodiments, a SARS-CoV-2 S protein may be stabilized by introducing one or more proline mutations. Examples of such proline mutations are known in the art, and include, e.g., mutations at positions corresponding to F817P, L865P, T866P, A892P, A899P, T912P, A893P, E895P, K921P, K922P, N978P, A942P, G946P, S975P, K986P, and V987P (positions relative to SEQ ID NO:1). In some embodiments, a SARS-CoV-2 S protein comprises a proline substitution at positions corresponding to residues 986 and/or 987 of SEQ ID NO: 1. In some embodiments, a SARS-CoV-2 S protein comprises a proline substitution at one or more positions corresponding to residues 817, 892, 899, and 942 of SEQ ID NO: 1. In some embodiments, a SARS-CoV-2 S protein comprises a proline substitution at positions corresponding to each of residues 817, 892, 899, and 942 of SEQ ID NO: 1. In some embodiments, a SARS-CoV-2 S protein comprises a proline substitution at positions corresponding to each of residues 817, 892, 899, 942, 986, and 987 of SEQ ID NO: 1.

In some embodiments, a Spike protein can be modified in such a way as to block a pre-fusion to post-fusion conformational change (referred to herein as a “pre-post fusion block”) and/or to reduce shedding. In some embodiments, a pre-post fusion block can be introduced or shedding reduced by introducing one or more pairs of cysteine mutations (e.g., at locations close to one another in a pre-fusion conformation of the Spike protein).

Examples of locations for such pairs of cysteine mutations are known in the art, and include, pairs at positions corresponding to, e.g., S383C/D985C, G413C/P987C, A668C and V963C or P862C, I712C/T1077C, T547C/N978C; K558C/N282C, A570C/V963C, D571C/S967C, A653C/A694C, S659C/S698C, C662C/M697C, A668C/P862C, A672C/A694C; V705C/A983C; V705C/T883C, Y707C/T883C, 1714C/Y1110C, P715C/P1069C, V722C/A930C; L727C/S1021C; P728C/V951C; V729C/A1022C; S735C/T859C; V736C/L858C; I770C/A1015C; T791C/A879C; G799C/A924C; P807C/S875C; E819C/Q1054C, E819C/S1055C; L822C/A1056C; 1870C/S1055C; T874C/S1055C; S884C/A893C; G885C/Q901C; G889C/L1034C, A890C/V1040C; 1896C/Q901C; A903C/Q913C; 1909C/Y1047C; V911C/N1108C; N914C/S1123C; T961C/S758C; T961C/E762C; Q965C/S1003C; F970C/G999C; A972C/1980C; A972C/Q992C; A972C/R995C; S974C/D979C; A1078C/V1133C; A1080C/I1132C; I1081C/N1135C; H1088C/T1120C; F1103C/P1112C; T1116C/Y1138C; and T1117C/D1139C (position relative to SEQ ID NO: 1). Further disclosure regarding exemplary disulfide bond mutations are disclosed, in e.g., US 2023/0021583 A1, WO 2022/241229 A1, WO 2022/040220 A1, and WO 2021/243122 A1 the contents of which are incorporated by reference herein in their entirety.

In some embodiments, a prefusion confirmation of an S protein can be stabilized by introducing one or more “cavity filling” mutations. A “cavity filling mutation” refers to an amino acid substitution that fills a cavity within the core of a folded protein. Cavities are voids within a folded protein where amino acids or amino acid side chains are not present (as determined, e.g., by examination of structural models of a SARS-CoV-2 S protein). In some embodiments, a prefusion conformation of a SARS-CoV-2 S protein can be stabilized by introducing a mutation that fills a cavity present in the prefusion conformation of a SARS-CoV-2 S protein that collapses (e.g., has a reduced volume) after transition to the postfusion conformation. Suitable cavity filling mutations are known in the art, and include, e.g., mutations at positions corresponding to T724I, T724M, S730L, T778L, Q779M, V826L, S875F, T887W, A890V, L894F, A899F, Q901M, L923W, P937F, L938F, M939K, A944F, A944Y, V963L, S975M, V9831, R1000Y, R1000W, S1003V, I1013F, A1016, A1020, A1020W, L1033, R1039F, V1040F, V1040Y, V1041, H1058W, H1058F, H1058Y, P1069F, H1088W, H1088Y, D1118F, N1132Y, and L1141F, and any combination thereof (positions relative to SEQ ID NO: 1). Further disclosure regarding cavity filling mutations can be found in, e.g., WO 2023/015332 A1, WO 2023/001259 A1, WO 2022/241229 A1, and WO 2021/243122 A1, the contents of each of which are incorporated by reference herein in their entirety.

In some embodiments, a Spike protein can be modified so as to decrease “shedding” (i.e., decrease separation of S1 and S2 subunits). In some embodiments, a Spike protein can be modified to decrease shedding by introducing mutations at the furin cleavage site, such that a furin protease can no longer bind and/or cleave the S protein (e.g., one or more mutations at position corresponding to residues 682-685 of SEQ ID NO: 1). In some embodiments, an S protein can be modified to reduce shedding by introducing mutations at positions corresponding to residues 682 and 685 of SEQ ID NO: 1 (e.g., introducing mutations corresponding to R682S and R685G of SEQ ID NO: 1), mutations at positions corresponding to each of residues 682, 683, and 685 of SEQ ID NO: 1 (e.g., introducing mutations corresponding to (i) R682G, R683S, and R685S, (ii) R682G, R683S, and R685G, or (iii) R682Q, R683Q, and R685Q of SEQ ID NO: 1), or by introducing mutations at positions corresponding to each of residues 682, 683, 684, and 685 of SEQ ID NO: 1 (e.g., introducing mutations corresponding to R682G, R683S, A684G, and R685G of SEQ ID NO: 1).

In some embodiments, one or more modifications may be introduced into a Spike protein so as to stabilize an “up” conformation (referred to herein as “RBD Up” mutations). Without wishing to be bound by theory, the up conformation of the SARS-CoV-2 Spike protein is thought to increase exposure of neutralization sensitive residues. Thus, mutations that stabilize the up conformation can produce a vaccine that is more immunogenic.

In some embodiments, stabilization of the prefusion conformation may be obtained by introducing two consecutive proline substitutions at AS residues at positions corresponding to residues 986 and 987 of SEQ ID NO: 1 in the full length spike protein. Specifically, spike(S) protein stabilized protein variants can be obtained in a way that the amino acid residue at the residue corresponding to position 986 of SEQ ID NO: 1 is exchanged to proline and the amino acid residue at the position corresponding to position 987 of SEQ ID NO: 1 is also exchanged to proline. In one embodiment, a SARS-CoV-2 S protein variant wherein the prototypical prefusion conformation is stabilized comprises the amino acid sequence shown in SEQ ID NO: 7:

(SEQ ID NO: 7)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

Those skilled in the art are aware of various spike variants, and/or resources that document them. For example, the following strains, their SARS-CoV-2 S protein amino acid sequences and, in particular, modifications thereof compared to wildtype SARS-CoV-2 S protein amino acid sequence, e.g., as compared to SEQ ID NO: 1, are useful herein.

### B.1.1.7 (“Variant of Concern 202012/01” (VOC-202012/01)

B.1.1.7 (the “alpha variant) is a variant of SARS-CoV-2 which was first detected in October 2020 during the COVID-19 pandemic in the United Kingdom from a sample taken the previous month, and it quickly began to spread by mid-December. It is correlated with a significant increase in the rate of COVID-19 infection in United Kingdom; this increase is thought to be at least partly because of change N501Y inside the spike glycoprotein's receptor-binding domain, which is needed for binding to ACE2 in human cells. The B.1.1.7 variant is defined by 23 mutations: 13 non-synonymous mutations, 4 deletions, and 6 synonymous mutations (i.e., there are 17 mutations that change proteins and six that do not). The spike protein changes in B.1.1.7 include deletion 69-70, deletion 144, N501Y, A570D, D614G, P681H, T716I, S982A, and D1118H.

### B.1.351 (501.V2)

B.1.351 lineage (“the Beta variant”) and colloquially known as South African COVID-19 variant, is a variant of SARS-CoV-2. Preliminary results indicate that this variant may have an increased transmissibility. The B.1.351 variant is defined by multiple spike protein changes including: L18F, D80A, D215G, deletion 242-244, R246I, K417N, E484K, N501Y, D614G and A701V. There are three mutations of particular interest in the spike region of the B.1.351 genome: K417N, E484K, N501Y.

### B.1.1.298 (Cluster 5)

B.1.1.298 was discovered in North Jutland, Denmark, and is believed to have been spread from minks to humans via mink farms. Several different mutations in the spike protein of the virus have been confirmed. The specific mutations include deletion 69-70, Y453F, D614G, I692V, M1229I, and optionally S1147L.

### P.1 (B.1.1.248)

Lineage B.1.1.248 (the “gamma variant”), known as the Brazil(ian) variant, is one of the variants of SARS-CoV-2 which has been named P.1 lineage. P.1 has a number of S-protein modifications [L18F, T20N, P26S, D138Y, R190S, K417T, E484K, N501Y, D614G, H655Y, T1027I, V1176F] and is similar in certain key RBD positions (K417, E484, N501) to variant B.1.351 from South Africa.

### B.1.427/B.1.429 (CAL.20C)

Lineage B.1.427/B.1.429 (the “epsilon variant”), also known as CAL.20C, is defined by the following modifications in the S-protein: S13I, W152C, L452R, and D614G of which the L452R modification is of particular concern. CDC has listed B.1.427/B.1.429 as “variant of concern”.

### B.1.525

B.1.525 (the “eta variant”) carries the same E484K modification as found in the P.1, and B.1.351 variants, and also carries the same ΔH69/ΔV70 deletion as found in B.1.1.7, and B.1.1.298. It also carries the modifications D614G, Q677H and F888L.

### B.1.526

B.1.526 (the “iota variant”) was detected as an emerging lineage of viral isolates in the New York region that shares mutations with previously reported variants. The most common sets of spike mutations in this lineage are L5F, T95I, D253G, E484K, D614G, and A701V.

The following table shows an overview of exemplary SARS-CoV-2 strains which are or which have been VOI/VOC.

TABLE 1
Overview of certain circulating SARS-CoV-2 strains which have been or are VOI/VOC

| Lineage | Amino acid substitution | | | | | | | | | | |
|---|---|---|---|---|---|---|---|---|---|---|---|
| P.1 (BRA) | | | L18F | T20N | P26S | | | | D138Y | | |
| B.1.1.7 (UK) | | | | | | ΔH69/V70 | | | | ΔY144 | |
| B.1.351 (SA) | | | L18F | | | | D80A | | | | |
| B.1.1.298 (DK) | | | | | | ΔH69/V70 | | | | | |
| B.1.427/B.1.429 (CAL) | | S13I | | | | | | | | | W152C |
| B.1.525 | | | | | | ΔH69/V70 | | | | | |
| B.1.526 (NY) | L5F | | | | | | | T95I | | | |

| Lineage | Amino acid substitution | | | | | | | | | | |
|---|---|---|---|---|---|---|---|---|---|---|---|
| P.1 (BRA) | R190S | | | | | K417T | | | E484K | N501Y | |
| B.1.1.7 (UK) | | | | | | | | | | N501Y | A570D |
| B.1.351 (SA) | | D215G | Δ242/243/ | R246I | | K417N | | | E484K | N501Y | |
| B.1.1.298 (DK) | | | | | | | | Y453F | | | |
| B.1.427/B.1.429 (CAL) | | | | | | | L452R | | | | |
| B.1.525 | | | | | | | | | E484K | | |
| B.1.526 (NY) | | | | | D253G | | | | E484K | | |

| Lineage | Amino acid substitution | | | | | | |
|---|---|---|---|---|---|---|---|
| P.1 (BRA) | D614G | H655Y | | | | | |
| B.1.1.7 (UK) | D614G | | | P681H | | | T716I |
| B.1.351 (SA) | D614G | | | | | A701V | |
| B.1.1.298 (DK) | D614G | | | | I692V | | |
| B.1.427/B.1.429 (CAL) | D614G | | | | | | |
| B.1.525 | D614G | | Q677H | | | | |
| B.1.526 (NY) | D614G | | | | | A701V | |

| Lineage | Amino acid substitution | | | | | |
|---|---|---|---|---|---|---|
| P.1 (BRA) | | | T1027I | | V1176F | |
| B.1.1.7 (UK) | | S982A | | D1118H | | |
| B.1.351 (SA) | | | | | | |
| B.1.1.298 (DK) | | | | | | M1229I |
| B.1.427/B.1.429 (CAL) | | | | | | |
| B.1.525 | F888L | | | | | |
| B.1.526 (NY) | | | | | | |

### B.1.1.529

B.1.529 (the “Omicron variant”) was first detected in South Africa in November 2021. Omicron multiplies around 70 times faster than Delta variants, and quickly became the dominant strain of SARS-CoV-2 worldwide. Since its initial detection, a number of Omicron sublineages have arisen. Listed below are Omicron variants of concern, along with certain characteristic mutations associated with the S protein of each. The S protein of BA.4 and BA.5 have the same set of characteristic mutations, which is why the below table has a single row for “BA.4 or BA.5”, and why the present disclosure refers to a “BA.4/5” S protein in some embodiments. Similarly, the S proteins of the BA.4.6 and BF.7 Omicron variants have the same set of characteristic mutations, which is why the below table has a single row for “BA.4.6 or BF.7”).

TABLE 2
Characteristic mutations of certain Omicron variants of concern

| Subvariant | Characteristic mutations |
|---|---|
| BA.1 | A67V, Δ69-70, T95I, G142D, Δ143-145, Δ211, L212I, ins214EPE, G339D, |
| | S371L, S373P, S375F, K417N, N440K, G446S, S477N, T478K, E484A, Q493R, |
| | G496S, Q498R, N501Y, Y505H, T547K, D614G, H655Y, N679K, P681H, |
| | N764K, D796Y, N856K, Q954H, N969K, and L981F |
| BA.2 | T19I, Δ24-26, A27S, G142D, V213G, G339D, S371F, S373P, S375F, T376A, |
| | D405N, R408S, K417N, N440K, S477N, T478K, E484A, Q493R, Q498R, N501Y, |
| | Y505H, D614G, H655Y, N679K, P681H, N764K, D796Y, Q954H, and N969K |
| BA.2.12.1 | T19I, Δ24-26, A27S, G142D, V213G, G339D, S371F, S373P, S375F, T376A, |
| | D405N, R408S, K417N, N440K, L452Q, S477N, T478K, E484A, Q493R, Q498R, |
| | N501Y, Y505H, D614G, H655Y, N679K, P681H, S704L, N764K, D796Y, Q954H, |
| | and N969K |
| BA.2.12.1 | T19I, Δ24-26, A27S, G142D, V213G, G339D, S371F, S373P, S375F, T376A, |
| | D405N, R408S, K417N, N440K, L452Q, S477N, T478K, E484A, Q493R, Q498R, |
| | N501Y, Y505H, D614G, H655Y, N679K, P681H, S704L, N764K, D796Y, Q954H, |
| | N969K |
| BA.4 or BA.5 | Characteristic mutations (1): T19I, Δ24-26, A27S, Δ69/70, G142D, V213G, |
| | G339D, S371F, S373P, S375F, T376A, D405N, R408S, K417N, N440K, L452R, |
| | S477N, T478K, E484A, F486V, Q498R, N501Y, Y505H, D614G, H655Y, N679K, |
| | P681H, N764K, D796Y, Q954H, and N969K |
| | Characteristic mutations (2): T19I, Δ24-26, A27S, Δ69/70, G142D, V213G, |
| | G339D, S371F, S373P, S375F, T376A, D405N, K417N, N440K, L452R, S477N, |
| | T478K, E484A, F486V, Q498R, N501Y, Y505H, D614G, H655Y, N679K, P681H, |
| | N764K, D796Y, Q954H, and N969K |
| BA.2.75 | T19I, Δ24-26, A27S, G142D, K147E, W152R, F157L, I210V, V213G, G257S, |
| | G339H, N354D, S371F, S373P, S375F, T376A, D405N, R408S, K417N, N440K, |
| | G446S, N460K, S477N, T478K, E484A, Q498R, N501Y, Y505H D614G, H655Y, |
| | N679K, P681H, N764K, D796Y, Q954H, and N969K |
| BA.2.75.2 | T19I, Δ24-26, A27S, G142D, K147E, W152R, F157L, I210V, V213G, G257S, |
| | G339H, R346T, N354D, S371F, S373P, S375F, T376A, D405N, R408S, K417N, |
| | N440K, G446S, N460K, S477N, T478K, E484A, F486S, Q498R, N501Y, Y505H |
| | D614G, H655Y, N679K, P681H, N764K, D796Y, Q954H, N969K, and D1199N |
| BJ.1 | T19I, Δ24-26, A27S, V83A, G142D, Δ144, H146Q, Q183E, V213E, G339H, |
| | R346T, L368I, S371F, S373P, S375F, T376A, D405N, R408S, K417N, N440K, |
| | V445P, G446S, S477N, T478K, V483A, E484A, F490V, Q493R, Q498R, N501Y, |
| | Y505H, D614G, H655Y, N679K, P681H, N764K, D796Y, G798D, Q954H, |
| | N969K, and S1003I |
| BA.4.6 or | T19I, Δ24-26, A27S, Δ69/70, G142D, V213G, G339D, R346T, S371F, S373P, |
| BF.7 | S375F, T376A, D405N, R408S, K417N, N440K, L452R, S477N, T478K, E484A, |
| | F486V, Q498R, N501Y, Y505H, D614G, H655Y, N658S, N679K, P681H, N764K, |
| | D796Y, Q954H, and N969K |
| XBB | Characteristic mutations (1): T19I, Δ24-26, A27S, V83A, G142D, Δ144, H146Q, |
| | Q183E, V213E, G339H, R346T, L368I, S371F, S373P, S375F, T376A, D405N, |
| | R408S, K417N, N440K, V445P, G446S, N460K, S477N, T478K, E484A, F486S, |
| | F490S, Q493R, Q498R, N501Y, Y505H, D614G, H655Y, N679K, P681H, N764K, |
| | D796Y, Q954H, and N969K |
| | Characteristic mutations (2): T19I, Δ24-26, A27S, V83A, G142D, Δ145, H146Q, |
| | Q183E, V213E, G339H, R346T, L368I, S371F, S373P, S375F, T376A, D405N, |
| | R408S, K417N, N440K, V445P, G446S, N460K, S477N, T478K, E484A, F486S, |
| | F490S, Q498R, N501Y, Y505H, D614G, H655Y, N679K, P681H, N764K, D796Y, |
| | Q954H, and N969K |
| XBB.1 | Characteristic mutations (1): T19I, Δ24-26, A27S, V83A, G142D, Δ144, H146Q, |
| | Q183E, V213E, G252V, G339H, R346T, L368I, S371F, S373P, S375F, T376A, |
| | D405N, R408S, K417N, N440K, V445P, G446S, N460K, S477N, T478K, E484A, |
| | F486S, F490S, Q493R, Q498R, N501Y, Y505H, D614G, H655Y, N679K, P681H, |
| | N764K, D796Y, Q954H, and N969K |
| | Characteristic mutations (2): T19I, Δ24-26, A27S, V83A, G142D, Δ144, H146Q, |
| | Q183E, V213E, G339H, R346T, L368I, S371F, S373P, S375F, T376A, D405N, |
| | R408S, K417N, N440K, V445P, G446S, N460K, S477N, T478K, E484A, F486S, |
| | F490S, Q498R, N501Y, Y505H, D614G, H655Y, N679K, P681H, N764K, D796Y, |
| | Q954H, and N969K |
| XBB.2 | T19I, Δ24-26, A27S, V83A, G142D, Δ144, H146Q, Q183E, V213E, D253G, |
| | G339H, R346T, L368I, S371F, S373P, S375F, T376A, D405N, R408S, K417N, |
| | N440K, V445P, G446S, N460K, S477N, T478K, E484A, F486S, F490S, Q493R, |
| | Q498R, N501Y, Y505H, D614G, H655Y, N679K, P681H, N764K, D796Y, |
| | Q954H, and N969K |
| XBB.2.3 | T19I, Δ24-26, A27S, V83A, G142D, Δ144, H146Q, Q183E, V213E, D253G, |
| | G339H, R346T, L368I, S371F, S373P, S375F, T376A, D405N, R408S, K417N, |
| | N440K, V445P, G446S, N460K, S477N, T478K, E484A, F486P, F490S, Q498R, |
| | N501Y, P521S, D614G, H655Y, N679K, P681H, N764K, D796Y, Q954H, and |
| | N969K |
| XBB.2.3.2 | T19I, Δ24-26, A27S, V83A, G142D, Δ144, H146Q, Q183E, G184V, V213E, |
| | D253G, G339H, R346T, L368I, S371F, S373P, S375F, T376A, D405N, R408S, |
| | K417N, N440K, V445P, G446S, N460K, S477N, T478K, E484A, F486P, F490S, |
| | Q498R, N501Y, P521S, D614G, H655Y, N679K, P681H, N764K, D796Y, Q954H, |
| | and N969K |
| XBB.1.3 | T19I, Δ24-26, A27S, V83A, G142D, Δ144, H146Q, Q183E, V213E, G252V, |
| | G339H, R346T, L368I, S371F, S373P, S375F, T376A, D405N, R408S, K417N, |
| | N440K, V445P, G446S, N460K, S477N, T478K, A484T, F486S, F490S, Q493R, |
| | Q498R, N501Y, Y505H, D614G, H655Y, N679K, P681H, N764K, D796Y, |
| | Q954H, and N969K |
| XBB.4 | T19I, Δ24-26, A27S, V83A, G142D, Δ144, H146Q, Q183E, V213E, G252V, |
| | G339H, R346T, L368I, S371F, S373P, S375F, T376A, D405N, R408S, K417N, |
| | N440K, K444R, V445P, G446S, N460K, S477N, T478K, E484A, F486S, F490S, |
| | Q493R, Q498R, N501Y, Y505H, D614G, H655Y, N679K, P681H, N764K, D796Y, |
| | Q954H, and N969K |
| XBB.1.5 | Characteristic mutations (1): T19I, Δ24-26, A27S, V83A, G142D, Δ144, H146Q, |
| | Q183E, V213E, G339H, R346T, L368I, S371F, S373P, S375F, T376A, D405N, |
| | R408S, K417N, N440K, V445P, G446S, N460K, S477N, T478K, E484A, F486P, |
| | F490S, Q493R, Q498R, N501Y, Y505H, D614G, H655Y, N679K, P681H, N764K, |
| | D796Y, Q954H, and N969K |
| | Characteristic mutations (2): T19I, Δ24-26, A27S, V83A, G142D, Δ145, H146Q, |
| | Q183E, V213E, G252V, G339H, R346T, L368I, S371F, S373P, S375F, T376A, |
| | D405N, R408S, K417N, N440K, V445P, G446S, N460K, S477N, T478K, E484A, |
| | F486P, F490S, Q498R, N501Y, Y505H, D614G, H655Y, N679K, P681H, N764K, |
| | D796Y, Q954H, and N969K |
| XBB.1.16 | Characteristic mutations (1): T19I, Δ24-26, A27S, V83A, G142D, Δ144, H146Q, |
| | E180V, Q183E, V213E, G252V, G339H, R346T, L368I, S371F, S373P, S375F, |
| | T376A, D405N, R408S, K417N, N440K, V445P, G446S, N460K, S477N, T478R, |
| | E484A, F486P, F490S, Q498R, N501Y, Y505H, D614G, H655Y, N679K, P681H, |
| | N764K, D796Y, Q954H, and N969K |
| | Characteristic mutations (2): T19I, Δ24-26, A27S, V83A, G142D, Δ145, H146Q, |
| | E180V, Q183E, V213E, G252V, G339H, R346T, L368I, S371F, S373P, S375F, |
| | T376A, D405N, R408S, K417N, N440K, V445P, G446S, N460K, S477N, T478R, |
| | E484A, F486P, F490S, Q498R, N501Y, Y505H, D614G, H655Y, N679K, P681H, |
| | N764K, D796Y, Q954H, and N969K |
| BA.2.3.20 | T19I, Δ24-26, A27S, G142D, M153T, N164K, V213G, H245N, G257D, G339D, |
| | S371F, S373P, S375F, T376A, D405N, R408S, K417N, N440K, K444R, E484R |
| | N450D, L452M, N460K, S477N, T478K, E484A, Q493R, Q498R, N501Y, Y505H, |
| | D614G, H655Y, N679K, P681H, N764K, D796Y, Q954H, and N969K |
| BQ.1 | T191, Δ24-26, A27S, Δ69/70, G142D, V213G, G339D, S371F, S373P, S375F, |
| | T376A, D405N, R408S, K417N, N440K, K444T, L452R, N460K, S477N, T478K, |
| | E484A, F486V, Q498R, N501Y, Y505H, D614G, H655Y, N679K, P681H, N764K, |
| | D796Y, Q954H, N969K |
| BQ.1.1 | T191, Δ24-26, A27S, Δ69/70, G142D, V213G, G339D, R346T, S371F, S373P, |
| | S375F, T376A, D405N, R408S, K417N, N440K, K444T, L452R, N460K, S477N, |
| | T478K, E484A, F486V, Q498R, N501Y, Y505H, D614G, H655Y, N679K, P681H, |
| | N764K, D796Y, Q954H, N969K |
| BN.1 | T191, Δ24-26, A27S, G142D, V213G, G339D, R346T, K356T, S371F, S373P, |
| | S375F, T376A, D405N, R408S, K417N, N440K, G446S, N460K, R493Q, S477N, |
| | T478K, E484A, Q493R, Q498R, N501Y, Y505H, D614G, H655Y, N679K, P681H, |
| | N764K, D796Y, Q954H, and N969K |

In addition to the above Omicron variants, further variants of BA.5 have been observed (such variants including, e.g., BF.7, BF.14, BQ.1, and BQ.1.1) comprising one of more of the following mutations in the S protein (positions shown relative to SEQ ID NO: 1): E340X (e.g., E340K), R346X (e.g., R346T, R346I, or R346S), K444X (e.g., K444N or K444T), V445X, N450D, and N460X (e.g., N460K).

In some embodiments, RNA described herein comprises a nucleotide sequence encoding a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of an Omicron variant (e.g., one or more (e.g., all of the) mutations associated with a given Omicron variant in Table 2). In some embodiments, such an RNA further comprise one or more mutations that stabilize the S protein in a pre-fusion confirmation (e.g., in some embodiments, such RNA further comprises proline residues at positions corresponding to residues 986 and 987 of SEQ ID NO: 1). In some embodiments, an RNA comprises a nucleotide sequence encoding a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) listed in Table 2. In some such embodiments, an RNA comprises a nucleotide sequence encoding a SARS-CoV-2 S protein comprising one or more mutations from each of two or more variants listed in Table 2. In some embodiments, an RNA comprises a nucleotide sequence encoding a SARS-CoV-2 S protein comprising one or more (e.g., all) the mutations identified in Table 2 as being characteristic of a certain Omicron variant (e.g., in some embodiments, an RNA comprises a nucleotide sequence encoding a SARS-CoV-2 S protein comprising each of the mutations listed in Table 2 as being characteristic of an Omicron BA.1, BA.2, BA.2.12.1, BA.4/5, BA.2.75, BA.2.75.1, BA.4.6, BQ.1.1, XBB, XBB.1, XBB.2, XBB.1.3, XBB.1.5, XBB.1.16, XBB.2.3, XBB.2.3.2 variant).

In some embodiments, RNA described herein comprises a nucleotide sequence that encodes an immunogenic fragment of a SARS-CoV-2 S protein (e.g., the RBD), which comprises one or more mutations that are characteristic of a SARS-CoV-2 variant (e.g., an Omicron variant described herein). For example, in some embodiments, an RNA comprises a nucleotide sequence encoding the RBD of an S protein of a SARS-CoV-2 variant (e.g., a region of the S protein corresponding to amino acids 327 to 528 of SEQ ID NO: 1, and comprising one or more mutations that are characteristic of a variant of concern that lie within this region of the S protein).

In some embodiments, an RNA comprises a nucleotide sequence encoding the RBD of an XBB.1.5 SARS-CoV-2 variant, wherein (i) the RBD comprises amino acids 323 to 524 of SEQ ID NO: 129 or an amino acid sequence that is at least 70% (e.g., at least 80%, at least 85%, at least 90%, at least 95%, at least 96%, at least 97%, at least 98%, at least 99%) identical to SEQ ID NO: 129 and/or (ii) the RNA comprises nucleotides 967 to 1572 of SEQ ID NO: 131 or a nucleotide sequence that is at least 70% (e.g., at least 80%, at least 85%, at least 90%, at least 95%, at least 96%, at least 97%, at least 98%, at least 99%) identical to nucleotides 967 to 1572 of SEQ ID NO: 131131.

In some embodiments, RNA comprises a nucleotide sequence encoding the RBD of an XBB.1.16 SARS-CoV-2 variant, wherein (i) the RBD comprises amino acids 323 to 524 of SEQ ID NO: 134 or an amino acid sequence that is at least 70% (e.g., at least 80%, at least 85%, at least 90%, at least 95%, at least 96%, at least 97%, at least 98%, at least 99%) identical to SEQ ID NO: 134 and/or (ii) the RNA comprises nucleotides 967 to 1572 of SEQ ID NO: 136 or a nucleotide sequence that is at least 70% (e.g., at least 80%, at least 85%, at least 90%, at least 95%, at least 96%, at least 97%, at least 98%, at least 99%) identical to nucleotides 967 to 1572 of SEQ ID NO: 136136. In some embodiments, an RNA comprises a nucleotide sequence encoding the RBD of an XBB.2.3 SARS-CoV-2 variant, wherein (i) the RBD comprises amino acids 323 to 524 of SEQ ID NO: 139 or an amino acid sequence that is at least 70% (e.g., at least 80%, at least 85%, at least 90%, at least 95%, at least 96%, at least 97%, at least 98%, at least 99%) identical to SEQ ID NO: 139 and/or (ii) the RNA comprises nucleotides 967 to 1572 of SEQ ID NO: 141 or a nucleotide sequence that is at least 70% (e.g., at least 80%, at least 85%, at least 90%, at least 95%, at least 96%, at least 97%, at least 98%, at least 99%) identical to nucleotides 967 to 1572 of SEQ ID NO: 141141.

In some embodiments, RNA comprises a nucleotide sequence encoding the RBD of an XBB.2.3.2 SARS-CoV-2 variant, wherein (i) the RBD comprises amino acids 323 to 524 of SEQ ID NO: 144 or an amino acid sequence that is at least 70% (e.g., at least 80%, at least 85%, at least 90%, at least 95%, at least 96%, at least 97%, at least 98%, at least 99%) identical to SEQ ID NO: 144 and/or (ii) the RNA comprises nucleotides 967 to 1572 of SEQ ID NO: 146 or a nucleotide sequence that is at least 70% (e.g., at least 80%, at least 85%, at least 90%, at least 95%, at least 96%, at least 97%, at least 98%, at least 99%) identical to nucleotides 967 to 1572 of SEQ ID NO: 146146.

In some embodiments, RNA comprises a nucleotide sequence encoding the RBD of an XBB.1.16 SARS-CoV-2 variant, wherein (i) the amino acid sequence of the RBD comprises amino acids 323 to 524 of SEQ ID NO: 134 and/or (ii) the RNA comprises a nucleotide sequence that is at least 70% (e.g., at least 80%, at least 85%, at least 90%, at least 95%, at least 96%, at least 97%, at least 98%, at least 99%) identical to nucleotides 967 to 1572 of SEQ ID NO: 135135.

In some embodiments, RNA encodes a SARS-CoV-2 S protein comprising a subset of the mutations listed in Table 2. In some embodiments, an RNA encodes a SARS-CoV-2 S protein comprising the mutations listed in Table 2 that are most prevalent in a certain variant (e.g., mutations that have been detected in at least 30%, at least 40%, at least 50%, at least 60%, at least 70%, at least 80%, at least 90%, at least 95%, at least 96%, at least 97%, at least 98%, at least 99%, or 100% of sequences collected to date for a given variant sequenced). Mutation prevalence can be determined, e.g., based on published sequences (e.g., sequences that are collected and made available to the public by GISAID).

In some embodiments, RNA described herein encodes a SARS-CoV-2 S protein comprising one or more mutations that are characteristic of a BA.4/5 variant. In some embodiments, the one or more mutations characteristic of a BA.4/5 variant include T19I, Δ24-26, A27S, Δ69/70, G142D, V213G, G339D, S371F, S373P, S375F, T376A, D405N, K417N, N440K, L452R, S477N, T478K, E484A, F486V, Q498R, N501Y, Y505H, D614G, H655Y, N679K, P681H, N764K, D796Y, Q954H, and N969K, or any combination thereof. In some embodiments, RNA described herein encodes a SARS-CoV-2 S protein comprising one or more mutations that are characteristic of a BA.4/5 variant and excludes R408S. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more (e.g., all)) that are characteristic of a BA.4/5 variant and excludes R408S.

In some embodiments, RNA described herein encodes a SARS-CoV-2 S protein comprising one or more (including, e.g., 2, 3, 4, 5, 6, 7, 8, 9, 10, 11, 12, 13, 14, 15, 16, 17, 18, 19, 20, or more (e.g., all)) mutations characteristic of a BA.2.75 variant. In some embodiments, the one or more mutations characteristic of a BA.2.75 variant include T19I, Δ24-26, A27S, G142D, K147E, W152R, F157L, I210V, V213G, G257S, G339H, S371F, S373P, S375F, T376A, D405N, R408S, K417N, N440K, G446S, N460K, S477N, T478K, E484A, Q498R, N501Y, Y505H D614G, H655Y, N679K, P681H, N764K, Q954H, and N969K, or any combination thereof. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BA.2.75 variant, and which excludes N354D. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BA.2.75 variant, and which excludes D796Y. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BA.2.75 variant, and which excludes D796Y and N354D.

In some embodiments, RNA described herein encodes a SARS-CoV-2 S protein comprising one or more mutations characteristic of a BA.2.75.2 variant. In some embodiments, the one or more mutations characteristic of a BA.2.75.2 variant include T19I, Δ24-26, A27S, G142D, K147E, W152R, F157L, 1210V, V213G, G257S, G339H, R346T, N354D,

S371F, S373P, S375F, T376A, D405N, R408S, K417N, N440K, G446S, N460K, S477N, T478K, E484A, F486S, Q498R, N501Y, Y505H, D614G, H655Y, N679K, P681H, N764K, D796Y, Q954H, N969K, and D1199N, or any combination thereof. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BA.2.75.2 variant, and which excludes R346T.

In some embodiments, RNA described herein encodes a SARS-CoV-2 S protein comprising one or more mutations characteristic of a BA.4.6 or BF.7 variant. In some embodiments, the one or more mutations characteristic of a BA.4.6 or BF.7 variant include T19I, Δ24-26, A27S, Δ69/70, G142D, V213G, G339D, R346T, S371F, S373P, S375F, T376A, D405N, K417N, N440K, L452R, S477N, T478K, E484A, F486V, Q498R, N501Y, Y505H, D614G, H655Y, N679K, P681H, N764K, D796Y, Q954H, and N969K, or any combination thereof. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BA.4.6 or BF.7 variant, and which exclude R408S. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BA.4.6 or BF.7 variant, and which exclude N658S. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BA.4.6 or BF.7 variant, and which exclude N658S and R408S.

In some embodiments, RNA described herein encodes a SARS-CoV-2 S protein comprising one or more mutations characteristic of an Omicron XBB variant. In some embodiments, the one or more mutations characteristic of an Omicron XBB variant include T19I, Δ24-26, A27S, V83A, G142D, Δ144, H146Q, Q183E, V213E, G339H, R346T, L368I, S371F, S373P, S375F, T376A, D405N, R408S, K417N, N440K, V445P, G446S, N460K, S477N, T478K, E484A, F486S, F490S, Q498R, N501Y, Y505H, D614G, H655Y, N679K, P681H, N764K, D796Y, Q954H, and N969K, or any combination thereof. In some embodiments, the one or more mutations characteristic of an Omicron XBB variant include T19I, Δ24-26, A27S, V83A, G142D, Δ144, H146Q, Q183E, V213E, G339H, R346T, L368I, S371F, S373P, S375F, T376A, D405N, R408S, K417N, N440K, V445P, G446S, N460K, S477N, T478K, E484A, F486S, F490S, Q498R, N501Y, Y505H, D614G, H655Y, N679K, P681H, N764K, D796Y, Q954H, and N969K, or any combination thereof. In some embodiments, the one or more mutations characteristic of an Omicron XBB variant include T19I, Δ24-26, A27S, V83A, G142D, Δ144, H146Q, Q183E, V213E, G339H, R346T, L368I, S371F, S373P, S375F, T376A, D405N, R408S, K417N, N440K, V445P, G446S, N460K, S477N, T478K, E484A, F486S, F490S, Q493R, Q498R, N501Y, Y505H, D614G, H655Y, N679K, P681H, N764K, D796Y, Q954H, and N969K, or any combination thereof. In some embodiments, the one or more mutations characteristic of an Omicron XBB variant include T19I, Δ24-26, A27S, V83A, G142D, Δ145, H146Q, Q183E, V213E, G339H, R346T, L3681, S371F, S373P, S375F, T376A, D405N, R408S, K417N, N440K, V445P, G446S, N460K, S477N, T478K, E484A, F486S, F490S, Q498R, N501Y, Y505H, D614G, H655Y, N679K, P681H, N764K, D796Y, Q954H, and N969K, or any combination thereof.

In some embodiments, RNA described herein encodes a SARS-CoV-2 S protein comprising one or more mutations characteristic of an Omicron XBB.1 variant. In some embodiments, the one or more mutations characteristic of an Omicron XBB.1 variant include G252V. In some embodiments, the one or more mutations characteristic of an Omicron XBB.1 variant include T19I, Δ24-26, A27S, V83A, G142D, Δ144, H146Q, Q183E, V213E, G252V, G339H, R346T, L368I, S371F, S373P, S375F, T376A, D405N, R408S, K417N, N440K, V445P, G446S, N460K, S477N, T478K, E484A, F486S, F490S, Q498R, N501Y, Y505H, D614G, H655Y, N679K, P681H, N764K, D796Y, Q954H, and N969K, or any combination thereof. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of an Omicron XBB.1 variant and which exclude Q493R. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of an Omicron XBB variant and which exclude Q493R and G252V.

In some embodiments, RNA described herein encodes a SARS-CoV-2 S protein comprising one or more mutations characteristic of an Omicron XBB.2 variant. In some embodiments, the one or more mutations characteristic of an Omicron XBB.2 variant include D253G. In some embodiments, the one or more mutations characteristic of an Omicron-XBB.2 variant include T19I, Δ24-26, A27S, V83A, G142D, Δ144, H146Q, Q183E, V213E, D253G, G339H, R346T, L368I, S371F, S373P, S375F, T376A, D405N, R408S, K417N, N440K, V445P, G446S, N460K, S477N, T478K, E484A, F486S, F490S, Q493R, Q498R, N501Y, Y505H, D614G, H655Y, N679K, P681H, N764K, D796Y, Q954H, and N969K, or any combination thereof.

In some embodiments, RNA described herein encodes a SARS-CoV-2 S protein comprising one or more mutations characteristic of an Omicron XBB.2.3 variant (e.g., as listed in the above Table 2). In some embodiments, the one or more mutations characteristic of an Omicron XBB.2.3 variant include one or more mutations characteristic of an XBB variant and one or more of D253G, F486P, and P521S. In some embodiments, the one or more mutations characteristic of an Omicron XBB.2.3 variant include T19I, Δ24-26, A27S, V83A, G142D, Δ144, H146Q, Q183E, V213E, D253G, G339H, R346T, L368I, S371F, S373P, S375F, T376A, D405N, R408S, K417N, N440K, V445P, G446S, N460K, S477N, T478K, E484A, F486P, F490S, Q498R, N501Y, P521S, D614G, H655Y, N679K, P681H, N764K, D796Y, Q954H, and N969K, or any combination thereof.

In some embodiments, RNA described herein encodes a SARS-CoV-2 S protein comprising one or more mutations characteristic of an Omicron XBB.2.3.2 variant (e.g., as listed in the above Table 2). In some embodiments, the one or more mutations characteristic of an Omicron XBB.2.3.2 variant include one or more mutations characteristic of an XBB variant and one or more of G184V, D253G, F486P, and P521S. In some embodiments, the one or more mutations characteristic of an Omicron XBB.2.3 variant include T19I, Δ24-26, A27S, V83A, G142D, Δ144, H146Q, Q183E, G184V, V213E, D253G, G339H, R346T, L368I, S371F, S373P, S375F, T376A, D405N, R408S, K417N, N440K, V445P, G446S, N460K, S477N, T478K, E484A, F486P, F490S, Q498R, N501Y, P521S, D614G, H655Y, N679K, P681H, N764K, D796Y, Q954H, and N969K, or any combination thereof.

In some embodiments, RNA described herein encodes a SARS-CoV-2 S protein comprising one or more mutations characteristic of an Omicron XBB.1.3 variant. In some embodiments, the one or more mutations characteristic of an Omicron XBB.1.3 variant include G252V and A484T. In some embodiments, the one or more mutations characteristic of an Omicron XBB.1.3 variant include T19I, Δ24-26, A27S, V83A, G142D, Δ144, H146Q, Q183E, V213E, G252V, G339H, R346T, L368I, S371F, S373P, S375F, T376A, D405N, R408S, K417N, N440K, V445P, G446S, N460K, S477N, T478K, A484T, F486S, F490S, Q493R, Q498R, N501Y, Y505H, D614G, H655Y, N679K, P681H, N764K, D796Y, Q954H, and N969K, or any combination thereof.

In some embodiments, RNA described herein encodes a SARS-CoV-2 S protein comprising one or more mutations characteristic of an Omicron XBB.1.5 variant. In some embodiments, the one or more mutations characteristic of an Omicron XBB.1.5 variant include F486P. In some embodiments, the one or more mutations characteristic of an Omicron XBB.1.5 variant include T19I, Δ24-26, A27S, V83A, G142D, Δ144, H146Q, Q183E, V213E, G252V, G339H, R346T, L368I, S371F, S373P, S375F, T376A, D405N, R408S, K417N, N440K, V445P, G446S, N460K, S477N, T478K, E484A, S486P, F490S, Q498R, N501Y, Y505H, D614G, H655Y, N679K, P681H, N764K, D796Y, Q954H, and N969K, or any combination thereof. In some embodiments, the one or more mutations characteristic of an Omicron XBB.1.5 variant include T19I, Δ24-26, A27S, V83A, G142D, Δ144, H146Q, Q183E, V213E, G252V, G339H, R346T, L368I, S371F, S373P, S375F, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K, or any combination thereof.

In some embodiments, RNA described herein encodes a SARS-CoV-2 S protein comprising one or more mutations characteristic of an Omicron XBB.1.16 variant. In some embodiments, the one or more mutations characteristic of an Omicron XBB.1.16 variant include E180V and K478R. In some embodiments, the one or more mutations characteristic of an Omicron XBB.1.16 variant include T19I, Δ24-26, A27S, V83A, G142D, Δ144, H146Q, Q183E, V213E, G252V, G339H, R346T, L368I, S371F, S373P, S375F, T376A, D405N, R408S, K417N, N440K, V445P, G446S, N460K, S477N, T478K, E484A, S486P, F490S, Q498R, N501Y, Y505H, D614G, H655Y, N679K, P681H, N764K, D796Y, Q954H, and N969K, or any combination thereof. In some embodiments, the one or more mutations characteristic of an Omicron XBB.1.16 variant include T19I, Δ24-26, A27S, V83A, G142D, Δ145, H146Q, E180V, Q183E, V213E, G252V, G339H, R346T, L368I, S371F, S373P, S375F, T376A, D405N, R408S, K417N, N440K, V445P, G446S, N460K, S477N, T478R, E484A, F486P, F490S, Q498R, N501Y, Y505H, D614G, H655Y, N679K, P681H, N764K, D796Y, Q954H, and N969K, or any combination thereof.

In some embodiments, RNA described herein encodes a SARS-CoV-2 S protein comprising one or more mutations that are characteristic of a BQ.1.1 variant. In some embodiments, the one or more mutations characteristic of a BQ.1.1 variant include T19I, Δ24-26, A27S, Δ69/70, G142D, V213G, G339D, S371F, S373P, S375F, T376A, D405N, R408S, K417N, N440K, K444T, L452R, N463K, S477N, T478K, E484A, F486V, Q498R, N501Y, Y505H, D614G, H655Y, N679K, P681H, N764K, D796Y, Q954H, and N969K, or any combination thereof. In some embodiments, RNA described herein encodes a SARS-CoV-2 S protein comprising one or more mutations (including, e.g., 2, 3, 4, 5, 6, 7, 8, 9, 10, 11, 12, 13, 14, 15, 16, 17, 18, 19, 20, or more) that are characteristic of a BQ.1.1 variant.

In one embodiment, a vaccine antigen described herein comprises, consists essentially of or consists of a spike protein(S) of SARS-CoV-2, a variant thereof, or a fragment thereof.

In one embodiment, a vaccine antigen comprises the amino acid sequence of amino acids 17 to 1273 of SEQ ID NO: 1 or 7, an amino acid sequence having at least 99%, 98%, 97%, 96%, 95%, 90%, 85%, or 80% identity to the amino acid sequence of amino acids 17 to 1273 of SEQ ID NO: 1 or 7, or an immunogenic fragment of the amino acid sequence of amino acids 17 to 1273 of SEQ ID NO: 1 or 7, or the amino acid sequence having at least 99%, 98%, 97%, 96%, 95%, 90%, 85%, or 80% identity to the amino acid sequence of amino acids 17 to 1273 of SEQ ID NO: 1 or 7. In one embodiment, a vaccine antigen comprises the amino acid sequence of amino acids 17 to 1273 of SEQ ID NO: 1 or 7.

In one embodiment, RNA encoding a vaccine antigen (i) comprises the nucleotide sequence of nucleotides 49 to 3819 of SEQ ID NO: 2, 8 or 9, a nucleotide sequence having at least 99%, 98%, 97%, 96%, 95%, 90%, 85%, or 80% identity to the nucleotide sequence of nucleotides 49 to 3819 of SEQ ID NO: 2, 8 or 9, or a fragment of the nucleotide sequence of nucleotides 49 to 3819 of SEQ ID NO: 2, 8 or 9, or the nucleotide sequence having at least 99%, 98%, 97%, 96%, 95%, 90%, 85%, or 80% identity to the nucleotide sequence of nucleotides 49 to 3819 of SEQ ID NO: 2, 8 or 9; and/or (ii) encodes an amino acid sequence comprising the amino acid sequence of amino acids 17 to 1273 of SEQ ID NO: 1 or 7, an amino acid sequence having at least 99%, 98%, 97%, 96%, 95%, 90%, 85%, or 80% identity to the amino acid sequence of amino acids 17 to 1273 of SEQ ID NO: 1 or 7, or an immunogenic fragment of the amino acid sequence of amino acids 17 to 1273 of SEQ ID NO: 1 or 7, or the amino acid sequence having at least 99%, 98%, 97%, 96%, 95%, 90%, 85%, or 80% identity to the amino acid sequence of amino acids 17 to 1273 of SEQ ID NO: 1 or 7. In one embodiment, RNA encoding a vaccine antigen (i) comprises the nucleotide sequence of nucleotides 49 to 3819 of SEQ ID NO: 2, 8 or 9; and/or (ii) encodes an amino acid sequence comprising the amino acid sequence of amino acids 17 to 1273 of SEQ ID NO: 1 or 7.

In one embodiment, a vaccine antigen comprises, consists essentially of or consists of SARS-CoV-2 spike S1 fragment (S1) (the S1 subunit of a spike protein(S) of SARS-CoV-2), a variant thereof, or a fragment thereof.

In one embodiment, a vaccine antigen comprises the amino acid sequence of amino acids 17 to 683 of SEQ ID NO: 1, an amino acid sequence having at least 99%, 98%, 97%, 96%, 95%, 90%, 85%, or 80% identity to the amino acid sequence of amino acids 17 to 683 of SEQ ID NO: 1, or an immunogenic fragment of the amino acid sequence of amino acids 17 to 683 of SEQ ID NO: 1, or the amino acid sequence having at least 99%, 98%, 97%, 96%, 95%, 90%, 85%, or 80% identity to the amino acid sequence of amino acids 17 to 683 of SEQ ID NO: 1. In one embodiment, a vaccine antigen comprises the amino acid sequence of amino acids 17 to 683 of SEQ ID NO: 1.

In one embodiment, RNA encoding a vaccine antigen (i) comprises the nucleotide sequence of nucleotides 49 to 2049 of SEQ ID NO: 2, 8 or 9, a nucleotide sequence having at least 99%, 98%, 97%, 96%, 95%, 90%, 85%, or 80% identity to the nucleotide sequence of nucleotides 49 to 2049 of SEQ ID NO: 2, 8 or 9, or a fragment of the nucleotide sequence of nucleotides 49 to 2049 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2049 of SEQ ID NO: 2, 8 or 9; and/or (ii) encodes an amino acid sequence comprising the amino acid sequence of amino acids 17 to 683 of SEQ ID NO: 1, an amino acid sequence having at least 99%, 98%, 97%, 96%, 95%, 90%, 85%, or 80% identity to the amino acid sequence of amino acids 17 to 683 of SEQ ID NO: 1, or an immunogenic fragment of the amino acid sequence of amino acids 17 to 683 of SEQ ID NO: 1, or the amino acid sequence having at least 99%, 98%, 97%, 96%, 95%, 90%, 85%, or 80% identity to the amino acid sequence of amino acids 17 to 683 of SEQ ID NO: 1. In one embodiment, RNA encoding a vaccine antigen (i) comprises the nucleotide sequence of nucleotides 49 to 2049 of SEQ ID NO: 2, 8 or 9; and/or (ii) encodes an amino acid sequence comprising the amino acid sequence of amino acids 17 to 683 of SEQ ID NO: 1.

In one embodiment, a vaccine antigen comprises the amino acid sequence of amino acids 17 to 685 of SEQ ID NO: 1, an amino acid sequence having at least 99%, 98%, 97%, 96%, 95%, 90%, 85%, or 80% identity to the amino acid sequence of amino acids 17 to 685 of SEQ ID NO: 1, or an immunogenic fragment of the amino acid sequence of amino acids 17 to 685 of SEQ ID NO: 1, or the amino acid sequence having at least 99%, 98%, 97%, 96%, 95%, 90%, 85%, or 80% identity to the amino acid sequence of amino acids 17 to 685 of SEQ ID NO: 1. In one embodiment, a vaccine antigen comprises the amino acid sequence of amino acids 17 to 685 of SEQ ID NO: 1.

In one embodiment, RNA encoding a vaccine antigen (i) comprises the nucleotide sequence of nucleotides 49 to 2055 of SEQ ID NO: 2, 8 or 9, a nucleotide sequence having at least 99%, 98%, 97%, 96%, 95%, 90%, 85%, or 80% identity to the nucleotide sequence of nucleotides 49 to 2055 of SEQ ID NO: 2, 8 or 9, or a fragment of the nucleotide sequence of nucleotides 49 to 2055 of SEQ ID NO: 2, 8 or 9, or the nucleotide sequence having at least 99%, 98%, 97%, 96%, 95%, 90%, 85%, or 80% identity to the nucleotide sequence of nucleotides 49 to 2055 of SEQ ID NO: 2, 8 or 9; and/or (ii) encodes an amino acid sequence comprising the amino acid sequence of amino acids 17 to 685 of SEQ ID NO: 1, an amino acid sequence having at least 99%, 98%, 97%, 96%, 95%, 90%, 85%, or 80% identity to the amino acid sequence of amino acids 17 to 685 of SEQ ID NO: 1, or an immunogenic fragment of the amino acid sequence of amino acids 17 to 685 of SEQ ID NO: 1, or the amino acid sequence having at least 99%, 98%, 97%, 96%, 95%, 90%, 85%, or 80% identity to the amino acid sequence of amino acids 17 to 685 of SEQ ID NO: 1. In one embodiment, RNA encoding a vaccine antigen (i) comprises the nucleotide sequence of nucleotides 49 to 2055 of SEQ ID NO: 2, 8 or 9; and/or (ii) encodes an amino acid sequence comprising the amino acid sequence of amino acids 17 to 685 of SEQ ID NO: 1.

In one embodiment, the vaccine antigen comprises, consists essentially of or consists of the receptor binding domain (RBD) of the S1 subunit of a spike protein(S) of SARS-CoV-2, a variant thereof, or a fragment thereof. The amino acid sequence of amino acids 327 to 528 of SEQ ID NO: 1, a variant thereof, or a fragment thereof is also referred to herein as “RBD” or “RBD domain”.

In one embodiment, a vaccine antigen comprises the amino acid sequence of amino acids 327 to 528 of SEQ ID NO: 1, an amino acid sequence having at least 99%, 98%, 97%, 96%, 95%, 90%, 85%, or 80% identity to the amino acid sequence of amino acids 327 to 528 of SEQ ID NO: 1, or an immunogenic fragment of the amino acid sequence of amino acids 327 to 528 of SEQ ID NO: 1, or the amino acid sequence having at least 99%, 98%, 97%, 96%, 95%, 90%, 85%, or 80% identity to the amino acid sequence of amino acids 327 to 528 of SEQ ID NO: 1. In one embodiment, a vaccine antigen comprises the amino acid sequence of amino acids 327 to 528 of SEQ ID NO: 1.

In one embodiment, RNA encoding a vaccine antigen (i) comprises the nucleotide sequence of nucleotides 979 to 1584 of SEQ ID NO: 2, 8 or 9, a nucleotide sequence having at least 99%, 98%, 97%, 96%, 95%, 90%, 85%, or 80% identity to the nucleotide sequence of nucleotides 979 to 1584 of SEQ ID NO: 2, 8 or 9, or a fragment of the nucleotide sequence of nucleotides 979 to 1584 of SEQ ID NO: 2, 8 or 9, or the nucleotide sequence having at least 99%, 98%, 97%, 96%, 95%, 90%, 85%, or 80% identity to the nucleotide sequence of nucleotides 979 to 1584 of SEQ ID NO: 2, 8 or 9; and/or (ii) encodes an amino acid sequence comprising the amino acid sequence of amino acids 327 to 528 of SEQ ID NO: 1, an amino acid sequence having at least 99%, 98%, 97%, 96%, 95%, 90%, 85%, or 80% identity to the amino acid sequence of amino acids 327 to 528 of SEQ ID NO: 1, or an immunogenic fragment of the amino acid sequence of amino acids 327 to 528 of SEQ ID NO: 1, or the amino acid sequence having at least 99%, 98%, 97%, 96%, 95%, 90%, 85%, or 80% identity to the amino acid sequence of amino acids 327 to 528 of SEQ ID NO: 1. In one embodiment, RNA encoding a vaccine antigen (i) comprises the nucleotide sequence of nucleotides 979 to 1584 of SEQ ID NO: 2, 8 or 9; and/or (ii) encodes an amino acid sequence comprising the amino acid sequence of amino acids 327 to 528 of SEQ ID NO: 1.

### Signal Peptides

According to certain embodiments, a signal peptide is fused, either directly or through a linker, to a SARS-CoV-2 S protein, a variant thereof, or a fragment thereof, i.e., the antigenic peptide or protein. Accordingly, in one embodiment, a signal peptide is fused to the above described amino acid sequences derived from SARS-CoV-2 S protein or immunogenic fragments thereof (antigenic peptides or proteins) comprised by the vaccine antigens described above.

Such signal peptides are sequences, which typically exhibit a length of about 15 to 30 amino acids and are preferably located at the N-terminus of the antigenic peptide or protein, without being limited thereto. Signal peptides as defined herein preferably allow the transport of the antigenic peptide or protein as encoded by an RNA into a defined cellular compartment, preferably the cell surface, the endoplasmic reticulum (ER) or the endosomal-lysosomal compartment. In one embodiment, the signal peptide sequence as defined herein includes, without being limited thereto, the signal peptide sequence of SARS-CoV-2 S protein, in particular a sequence comprising the amino acid sequence of amino acids 1 to 16 or 1 to 19 of SEQ ID NO: 1 or a functional variant thereof.

In one embodiment, a signal sequence comprises the amino acid sequence of amino acids 1 to 16 of SEQ ID NO: 1, an amino acid sequence having at least 99%, 98%, 97%, 96%, 95%, 90%, 85%, or 80% identity to the amino acid sequence of amino acids 1 to 16 of SEQ ID NO: 1, or a functional fragment of the amino acid sequence of amino acids 1 to 16 of SEQ ID NO: 1, or the amino acid sequence having at least 99%, 98%, 97%, 96%, 95%, 90%, 85%, or 80% identity to the amino acid sequence of amino acids 1 to 16 of SEQ ID NO: 1. In one embodiment, a signal sequence comprises the amino acid sequence of amino acids 1 to 16 of SEQ ID NO: 1.

In one embodiment, RNA encoding a signal sequence (i) comprises the nucleotide sequence of nucleotides 1 to 48 of SEQ ID NO: 2, 8 or 9, a nucleotide sequence having at least 99%, 98%, 97%, 96%, 95%, 90%, 85%, or 80% identity to the nucleotide sequence of nucleotides 1 to 48 of SEQ ID NO: 2, 8 or 9, or a fragment of the nucleotide sequence of nucleotides 1 to 48 of SEQ ID NO: 2, 8 or 9, or the nucleotide sequence having at least 99%, 98%, 97%, 96%, 95%, 90%, 85%, or 80% identity to the nucleotide sequence of nucleotides 1 to 48 of SEQ ID NO: 2, 8 or 9; and/or (ii) encodes an amino acid sequence comprising the amino acid sequence of amino acids 1 to 16 of SEQ ID NO: 1, an amino acid sequence having at least 99%, 98%, 97%, 96%, 95%, 90%, 85%, or 80% identity to the amino acid sequence of amino acids 1 to 16 of SEQ ID NO: 1, or a functional fragment of the amino acid sequence of amino acids 1 to 16 of SEQ ID NO: 1, or the amino acid sequence having at least 99%, 98%, 97%, 96%, 95%, 90%, 85%, or 80% identity to the amino acid sequence of amino acids 1 to 16 of SEQ ID NO: 1. In one embodiment, RNA encoding a signal sequence (i) comprises the nucleotide sequence of nucleotides 1 to 48 of SEQ ID NO: 2, 8 or 9; and/or (ii) encodes an amino acid sequence comprising the amino acid sequence of amino acids 1 to 16 of SEQ ID NO: 1.

In one embodiment, a signal sequence comprises the amino acid sequence of amino acids 1 to 19 of SEQ ID NO: 1, an amino acid sequence having at least 99%, 98%, 97%, 96%, 95%, 90%, 85%, or 80% identity to the amino acid sequence of amino acids 1 to 19 of SEQ ID NO: 1, or a functional fragment of the amino acid sequence of amino acids 1 to 19 of SEQ ID NO: 1, or the amino acid sequence having at least 99%, 98%, 97%, 96%, 95%, 90%, 85%, or 80% identity to the amino acid sequence of amino acids 1 to 19 of SEQ ID NO: 1. In one embodiment, a signal sequence comprises the amino acid sequence of amino acids 1 to 19 of SEQ ID NO: 1.

In one embodiment, RNA encoding a signal sequence (i) comprises the nucleotide sequence of nucleotides 1 to 57 of SEQ ID NO: 2, 8 or 9, a nucleotide sequence having at least 99%, 98%, 97%, 96%, 95%, 90%, 85%, or 80% identity to the nucleotide sequence of nucleotides 1 to 57 of SEQ ID NO: 2, 8 or 9, or a fragment of the nucleotide sequence of nucleotides 1 to 57 of SEQ ID NO: 2, 8 or 9, or the nucleotide sequence having at least 99%, 98%, 97%, 96%, 95%, 90%, 85%, or 80% identity to the nucleotide sequence of nucleotides 1 to 57 of SEQ ID NO: 2, 8 or 9; and/or (ii) encodes an amino acid sequence comprising the amino acid sequence of amino acids 1 to 19 of SEQ ID NO: 1, an amino acid sequence having at least 99%, 98%, 97%, 96%, 95%, 90%, 85%, or 80% identity to the amino acid sequence of amino acids 1 to 19 of SEQ ID NO: 1, or a functional fragment of the amino acid sequence of amino acids 1 to 19 of SEQ ID NO: 1, or the amino acid sequence having at least 99%, 98%, 97%, 96%, 95%, 90%, 85%, or 80% identity to the amino acid sequence of amino acids 1 to 19 of SEQ ID NO: 1. In one embodiment, RNA encoding a signal sequence (i) comprises the nucleotide sequence of nucleotides 1 to 57 of SEQ ID NO: 2, 8 or 9; and/or (ii) encodes an amino acid sequence comprising the amino acid sequence of amino acids 1 to 19 of SEQ ID NO: 1.

The signal peptide sequence as defined herein further includes, without being limited thereto, the signal peptide sequence of an immunoglobulin, e.g., the signal peptide sequence of an immunoglobulin heavy chain variable region, wherein the immunoglobulin may be human immunoglobulin. In particular, the signal peptide sequence as defined herein includes a sequence comprising the amino acid sequence of amino acids 1 to 22 of SEQ ID NO: 31 or a functional variant thereof.

In some embodiments, a signal peptide sequence is functional in mammalian cells. In some embodiments, a utilized signal sequence is “intrinsic” in that it is, in nature, associated with (e.g., linked to) the encoded polypeptide. In some embodiments, a utilized signal sequence is heterologous to the encoded polypeptide—e.g., is not naturally part of a polypeptide (e.g., protein) whose sequences are included in the encoded polypeptide. In some embodiments, signal peptides are sequences, which are typically characterized by a length of about 15 to 30 amino acids. In many embodiments, signal peptides are positioned at the N-terminus of an encoded polypeptide as described herein, without being limited thereto. In some embodiments, signal peptides preferably allow the transport of the polypeptide encoded by RNAs of the present disclosure with which they are associated into a defined cellular compartment, preferably the cell surface, the endoplasmic reticulum (ER) or the endosomal-lysosomal compartment. In some embodiments, a signal sequence is selected from an S1S2 signal peptide (aa 1-19), an immunoglobulin secretory signal peptide (aa 1-22), an HSV-1 gD signal peptide (MGGAAARLGAVILFWVIVGLHGVRSKY (SEQ ID 110)), gD peptide an NO: HSV-2 signal (MGRLTSGVGTAALLVVAVGLRVVCA (SEQ ID NO: 111)); a human SPARC signal peptide, a human insulin isoform 1 signal peptide, a human albumin signal peptide, etc. Those skilled in the art will be aware of other secretory signal peptides such as, for example, as disclosed in WO2017/081082 (e.g., SEQ ID NOs: 1-1115 and 1728, or fragments variants thereof) and WO2019008001. In some embodiments, an RNA sequence encodes an epitope that may comprise or otherwise be linked to a signal sequence (e.g., secretory sequence), such as those listed in Table A, or at least a sequence having 1, 2, 3, 4, or 5 amino acid differences relative thereto. In some embodiments, a signal sequence such as MFVFLVLLPLVSSQCVNLT (SEQ ID NO: 113), or at least a sequence having 1, 2, 3, 4, or at the most 5 amino acid differences relative thereto is utilized. In some embodiments, a sequence such as MFVFLVLLPLVSSQCVNLT (SEQ ID NO: 113), or a sequence having 1, 2, 3, 4, or at the most 5 amino acid differences relative thereto, is utilized. In some embodiments, a signal sequence is selected from those included in the Table A below and/or those encoded by the sequences in Table B below:

TABLE A
Exemplary signal sequences

| SEQ | | |
|---|---|---|
| ID | | |
| NO: | Signal | Sequence (Amino Acid) |
| 110 | HSV-1 gD SP | MGGAAARLGAVILFVVIVGLHGVRSKY |
| 111 | HSV-2 gD SP | MGRLTSGVGTAALLVVAVGLRVVCA |
| 112 | HSV-2 | MGRLTSGVGTAALLVVAVGLRVVCAKYA |
| 113 | SARS-COV-2- | MFVFLVLLPLVSSQCVNLT |
| | S | |
| 114 | human Ig | MDWIWRILFLVGAATGAHSQM |
| | heavy chain | |
| | signal | |
| | peptide | |
| | (huSec) | |
| 115 | HuIgGk | METPAQLLFLLLLWLPDTTG |
| | signal | |
| | peptide | |
| 116 | IgE heavy | MDWTWILFLVAAATRVHS |
| | chain | |
| | epsilon- | |
| | 1signal | |
| | peptide | |
| 117 | Japanese | MLGSNSGQRVVFTILLLLVAPAYS |
| | encephalitis | |
| | PRM signal | |
| | sequence | |
| 118 | VSVg protein | MKCLLYLAFLFIGVNCA |
| | signal | |
| | sequence | |
| 119 | | MDWTWILFLVAAATRVHS |
| 120 | | ETPAQLLFLLLLWLPDTTG |
| 121 | | MLGSNSGQRVVFTILLLLVAPAYS |
| 122 | | MKCLLYLAFLFIGVNCA |
| 123 | | MWLVSLAIVTACAGA |
| 124 | | MFVFLVLLPLVSSQC |

TABLE B
Exemplary nucleotide sequences encoding
signal sequences

| SEQ | | |
|---|---|---|
| ID | | |
| NO: | Signal | Sequence (Nucleotide) |
| 125 | HSV-1 gD | ATGGGGGGGGCTGCCGCCAGGTTGGG |
| | SP wild-type | GGCCGTGATTTTGTTTGTCGTCATAG |
| | | TGGGCCTCCATGGGGTCCGCAGCAAA |
| | | TAT |
| 126 | HSV-1 gD SP | ATGggaggagccGCCGCCagactggg |
| | optimized | aGCCGTGatcctgttcgtggtgatcG |
| | nucleotide | TGggactgCATggagtgagaAGCaag |
| | sequence | tac |
| 127 | SARS-COV-2-S | ATGTTTGTGTTTCTTGTGCTGCTGCC |
| | | TCTTGTGTCTTCTCAGTGTGTGAATT |
| | | TGACA |
| 128 | human Ig | ATGGATTGGATTTGGAGAATCCTGTT |
| | heavy chain | CCTCGTGGGAGCCGCTACAGGAGCCC |
| | signal | ACTCCCAGATG |
| | peptide | |
| | (huSec) | |

### Multimerization Domains

In some embodiments, an RNA utilized as described herein encodes a multimerization element (e.g., a heterologous multimerization element). In some embodiments, a heterologous multimerization element comprises a dimerization, trimerization or tetramerization element. In some embodiments, a multimerization element is one described in WO2017/081082 (e.g., SEQ ID NOs: 1116-1167, or fragments or variants thereof). Exemplary trimerization and tetramerization elements include, but are not limited to, engineered leucine zippers, fibritin foldon domain from enterobacteria phage T4, GCN4pII, GCN4-pII, and p53. In some embodiments, a provided encoded polypeptide(s) is able to form a trimeric complex. For example, a utilized encoded polypeptide(s) may comprise a domain allowing formation of a multimeric complex, such as for example particular a trimeric complex of an amino acid sequence comprising an encoded polypeptide(s) as described herein. In some embodiments, a domain allowing formation of a multimeric complex comprises a trimerization domain, for example, a trimerization domain as described herein. In some embodiments, an encoded polypeptide(s) can be modified by addition of a T4-fibritin-derived “foldon” trimerization domain, for example, to increase its immunogenicity.

### Transmembrane Domains

In some embodiments, an RNA described herein encodes a membrane association element (e.g., a heterologous membrane association element), such as a transmembrane domain. A transmembrane domain can be N-terminal, C-terminal, or internal to an encoded polypeptide. A coding sequence of a transmembrane element is typically placed in frame (i.e., in the same reading frame), 5′, 3′, or internal to coding sequences of sequences (e.g., sequences encoding polypeptide(s)) with which it is to be linked. In some embodiments, a transmembrane domain comprises or is a transmembrane domain of Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, or a seven transmembrane domain receptor.

In one embodiment, a signal sequence comprises the amino acid sequence of amino acids 1 to 22 of SEQ ID NO: 31, an amino acid sequence having at least 99%, 98%, 97%, 96%, 95%, 90%, 85%, or 80% identity to the amino acid sequence of amino acids 1 to 22 of SEQ ID NO: 31, or a functional fragment of the amino acid sequence of amino acids 1 to 22 of SEQ ID NO: 31, or the amino acid sequence having at least 99%, 98%, 97%, 96%, 95%, 90%, 85%, or 80% identity to the amino acid sequence of amino acids 1 to 22 of SEQ ID NO: 31. In one embodiment, a signal sequence comprises the amino acid sequence of amino acids 1 to 22 of SEQ ID NO: 31.

In one embodiment, RNA encoding a signal sequence (i) comprises the nucleotide sequence of nucleotides 54 to 119 of SEQ ID NO: 32, a nucleotide sequence having at least 99%, 98%, 97%, 96%, 95%, 90%, 85%, or 80% identity to the nucleotide sequence of nucleotides 54 to 119 of SEQ ID NO: 32, or a fragment of the nucleotide sequence of nucleotides 54 to 119 of SEQ ID NO: 32, or the nucleotide sequence having at least 99%, 98%, 97%, 96%, 95%, 90%, 85%, or 80% identity to the nucleotide sequence of nucleotides 54 to 119 of SEQ ID NO: 32; and/or (ii) encodes an amino acid sequence comprising the amino acid sequence of amino acids 1 to 22 of SEQ ID NO: 31, an amino acid sequence having at least 99%, 98%, 97%, 96%, 95%, 90%, 85%, or 80% identity to the amino acid sequence of amino acids 1 to 22 of SEQ ID NO: 31, or a functional fragment of the amino acid sequence of amino acids 1 to 22 of SEQ ID NO: 31, or the amino acid sequence having at least 99%, 98%, 97%, 96%, 95%, 90%, 85%, or 80% identity to the amino acid sequence of amino acids 1 to 22 of SEQ ID NO: 31. In one embodiment, RNA encoding a signal sequence (i) comprises the nucleotide sequence of nucleotides 54 to 119 of SEQ ID NO: 32; and/or (ii) encodes an amino acid sequence comprising the amino acid sequence of amino acids 1 to 22 of SEQ ID NO: 31.

Such signal peptides are preferably used in order to promote secretion of the encoded antigenic peptide or protein. More preferably, a signal peptide as defined herein is fused to an encoded antigenic peptide or protein as defined herein.

Accordingly, in particularly preferred embodiments, RNA described herein comprises at least one coding region encoding an antigenic peptide or protein and a signal peptide, said signal peptide preferably being fused to the antigenic peptide or protein, more preferably to the N-terminus of the antigenic peptide or protein as described herein.

In one embodiment, a vaccine antigen comprises the amino acid sequence of SEQ ID NO: 1 or 7, an amino acid sequence having at least 99%, 98%, 97%, 96%, 95%, 90%, 85%, or 80% identity to the amino acid sequence of SEQ ID NO: 1 or 7, or an immunogenic fragment of the amino acid sequence of SEQ ID NO: 1 or 7, or the amino acid sequence having at least 99%, 98%, 97%, 96%, 95%, 90%, 85%, or 80% identity to the amino acid sequence of SEQ ID NO: 1 or 7. In one embodiment, a vaccine antigen comprises the amino acid sequence of SEQ ID NO: 1 or 7.

In one embodiment, RNA encoding a vaccine antigen (i) comprises the nucleotide sequence of SEQ ID NO: 2, 8 or 9, a nucleotide sequence having at least 99%, 98%, 97%, 96%, 95%, 90%, 85%, or 80% identity to the nucleotide sequence of SEQ ID NO: 2, 8 or 9, or a fragment of the nucleotide sequence of SEQ ID NO: 2, 8 or 9, or the nucleotide sequence having at least 99%, 98%, 97%, 96%, 95%, 90%, 85%, or 80% identity to the nucleotide sequence of SEQ ID NO: 2, 8 or 9; and/or (ii) encodes an amino acid sequence comprising the amino acid sequence of SEQ ID NO: 1 or 7, an amino acid sequence having at least 99%, 98%, 97%, 96%, 95%, 90%, 85%, or 80% identity to the amino acid sequence of SEQ ID NO: 1 or 7, or an immunogenic fragment of the amino acid sequence of SEQ ID NO: 1 or 7, or the amino acid sequence having at least 99%, 98%, 97%, 96%, 95%, 90%, 85%, or 80% identity to the amino acid sequence of SEQ ID NO: 1 or 7. In one embodiment, RNA encoding a vaccine antigen (i) comprises the nucleotide sequence of SEQ ID NO: 2, 8 or 9; and/or (ii) encodes an amino acid sequence comprising the amino acid sequence of SEQ ID NO: 1 or 7.

In one embodiment, a vaccine antigen comprises the amino acid sequence of SEQ ID NO: 7, an amino acid sequence having at least 99%, 98%, 97%, 96%, 95%, 90%, 85%, or 80% identity to the amino acid sequence of SEQ ID NO: 7, or an immunogenic fragment of the amino acid sequence of SEQ ID NO: 7, or the amino acid sequence having at least 99%, 98%, 97%, 96%, 95%, 90%, 85%, or 80% identity to the amino acid sequence of SEQ ID NO: 7. In one embodiment, a vaccine antigen comprises the amino acid sequence of SEQ ID NO: 7.

In one embodiment, RNA encoding a vaccine antigen (i) comprises the nucleotide sequence of SEQ ID NO: 15, 16, 19, 20, 24, or 25, a nucleotide sequence having at least 99%, 98%, 97%, 96%, 95%, 90%, 85%, or 80% identity to the nucleotide sequence of SEQ ID NO: 15, 16, 19, 20, 24, or 25, or a fragment of the nucleotide sequence of SEQ ID NO: 15, 16, 19, 20, 24, or 25, or the nucleotide sequence having at least 99%, 98%, 97%, 96%, 95%, 90%, 85%, or 80% identity to the nucleotide sequence of SEQ ID NO: 15, 16, 19, 20, 24, or 25; and/or (ii) encodes an amino acid sequence comprising the amino acid sequence of SEQ ID NO: 7, an amino acid sequence having at least 99%, 98%, 97%, 96%, 95%, 90%, 85%, or 80% identity to the amino acid sequence of SEQ ID NO: 7, or an immunogenic fragment of the amino acid sequence of SEQ ID NO: 7, or the amino acid sequence having at least 99%, 98%, 97%, 96%, 95%, 90%, 85%, or 80% identity to the amino acid sequence of SEQ ID NO: 7. In one embodiment, RNA encoding a vaccine antigen (i) comprises the nucleotide sequence of SEQ ID NO: 15, 16, 19, 20, 24, or 25; and/or (ii) encodes an amino acid sequence comprising the amino acid sequence of SEQ ID NO: 7.

In one embodiment, a vaccine antigen comprises the amino acid sequence of amino acids 1 to 683 of SEQ ID NO: 1, an amino acid sequence having at least 99%, 98%, 97%, 96%, 95%, 90%, 85%, or 80% identity to the amino acid sequence of amino acids 1 to 683 of SEQ ID NO: 1, or an immunogenic fragment of the amino acid sequence of amino acids 1 to 683 of SEQ ID NO: 1, or the amino acid sequence having at least 99%, 98%, 97%, 96%, 95%, 90%, 85%, or 80% identity to the amino acid sequence of amino acids 1 to 683 of SEQ ID NO: 1. In one embodiment, a vaccine antigen comprises the amino acid sequence of amino acids 1 to 683 of SEQ ID NO: 1.

In one embodiment, RNA encoding a vaccine antigen (i) comprises the nucleotide sequence of nucleotides 1 to 2049 of SEQ ID NO: 2, 8 or 9, a nucleotide sequence having at least 99%, 98%, 97%, 96%, 95%, 90%, 85%, or 80% identity to the nucleotide sequence of nucleotides 1 to 2049 of SEQ ID NO: 2, 8 or 9, or a fragment of the nucleotide sequence of nucleotides 1 to 2049 of SEQ ID NO: 2, 8 or 9, or the nucleotide sequence having at least 99%, 98%, 97%, 96%, 95%, 90%, 85%, or 80% identity to the nucleotide sequence of nucleotides 1 to 2049 of SEQ ID NO: 2, 8 or 9; and/or (ii) encodes an amino acid sequence comprising the amino acid sequence of amino acids 1 to 683 of SEQ ID NO: 1, an amino acid sequence having at least 99%, 98%, 97%, 96%, 95%, 90%, 85%, or 80% identity to the amino acid sequence of amino acids 1 to 683 of SEQ ID NO: 1, or an immunogenic fragment of the amino acid sequence of amino acids 1 to 683 of SEQ ID NO: 1, or the amino acid sequence having at least 99%, 98%, 97%, 96%, 95%, 90%, 85%, or 80% identity to the amino acid sequence of amino acids 1 to 683 of SEQ ID NO: 1. In one embodiment, RNA encoding a vaccine antigen (i) comprises the nucleotide sequence of nucleotides 1 to 2049 of SEQ ID NO: 2, 8 or 9; and/or (ii) encodes an amino acid sequence comprising the amino acid sequence of amino acids 1 to 683 of SEQ ID NO: 1.

In one embodiment, a vaccine antigen comprises the amino acid sequence of amino acids 1 to 685 of SEQ ID NO: 1, an amino acid sequence having at least 99%, 98%, 97%, 96%, 95%, 90%, 85%, or 80% identity to the amino acid sequence of amino acids 1 to 685 of SEQ ID NO: 1, or an immunogenic fragment of the amino acid sequence of amino acids 1 to 685 of SEQ ID NO: 1, or the amino acid sequence having at least 99%, 98%, 97%, 96%, 95%, 90%, 85%, or 80% identity to the amino acid sequence of amino acids 1 to 685 of SEQ ID NO: 1. In one embodiment, a vaccine antigen comprises the amino acid sequence of amino acids 1 to 685 of SEQ ID NO: 1.

In one embodiment, RNA encoding a vaccine antigen (i) comprises the nucleotide sequence of nucleotides 1 to 2055 of SEQ ID NO: 2, 8 or 9, a nucleotide sequence having at least 99%, 98%, 97%, 96%, 95%, 90%, 85%, or 80% identity to the nucleotide sequence of nucleotides 1 to 2055 of SEQ ID NO: 2, 8 or 9, or a fragment of the nucleotide sequence of nucleotides 1 to 2055 of SEQ ID NO: 2, 8 or 9, or the nucleotide sequence having at least 99%, 98%, 97%, 96%, 95%, 90%, 85%, or 80% identity to the nucleotide sequence of nucleotides 1 to 2055 of SEQ ID NO: 2, 8 or 9; and/or (ii) encodes an amino acid sequence comprising the amino acid sequence of amino acids 1 to 685 of SEQ ID NO: 1, an amino acid sequence having at least 99%, 98%, 97%, 96%, 95%, 90%, 85%, or 80% identity to the amino acid sequence of amino acids 1 to 685 of SEQ ID NO: 1, or an immunogenic fragment of the amino acid sequence of amino acids 1 to 685 of SEQ ID NO: 1, or the amino acid sequence having at least 99%, 98%, 97%, 96%, 95%, 90%, 85%, or 80% identity to the amino acid sequence of amino acids 1 to 685 of SEQ ID NO: 1. In one embodiment, RNA encoding a vaccine antigen (i) comprises the nucleotide sequence of nucleotides 1 to 2055 of SEQ ID NO: 2, 8 or 9; and/or (ii) encodes an amino acid sequence comprising the amino acid sequence of amino acids 1 to 685 of SEQ ID NO: 1.

In one embodiment, a vaccine antigen comprises the amino acid sequence of SEQ ID NO: 3, an amino acid sequence having at least 99%, 98%, 97%, 96%, 95%, 90%, 85%, or 80% identity to the amino acid sequence of SEQ ID NO: 3, or an immunogenic fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3. In one embodiment, a vaccine antigen comprises the amino acid sequence of SEQ ID NO: 3.

In one embodiment, RNA encoding a vaccine antigen (i) comprises the nucleotide sequence of SEQ ID NO: 4, a nucleotide sequence having at least 99%, 98%, 97%, 96%, 95%, 90%, 85%, or 80% identity to the nucleotide sequence of SEQ ID NO: 4, or a fragment of the nucleotide sequence of SEQ ID NO: 4, or the nucleotide sequence having at least 99%, 98%, 97%, 96%, 95%, 90%, 85%, or 80% identity to the nucleotide sequence of SEQ ID NO: 4; and/or (ii) encodes an amino acid sequence comprising the amino acid sequence of SEQ ID NO: 3, an amino acid sequence having at least 99%, 98%, 97%, 96%, 95%, 90%, 85%, or 80% identity to the amino acid sequence of SEQ ID NO: 3, or an immunogenic fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3. In one embodiment, RNA encoding a vaccine antigen (i) comprises the nucleotide sequence of SEQ ID NO: 4; and/or (ii) encodes an amino acid sequence comprising the amino acid sequence of SEQ ID NO: 3.

In one embodiment, a vaccine antigen comprises the amino acid sequence of amino acids 1 to 221 of SEQ ID NO: 29, an amino acid sequence having at least 99%, 98%, 97%, 96%, 95%, 90%, 85%, or 80% identity to the amino acid sequence of amino acids 1 to 221 of SEQ ID NO: 29, or an immunogenic fragment of the amino acid sequence of amino acids 1 to 221 of SEQ ID NO: 29, or the amino acid sequence having at least 99%, 98%, 97%, 96%, 95%, 90%, 85%, or 80% identity to the amino acid sequence of amino acids 1 to 221 of SEQ ID NO: 29. In one embodiment, a vaccine antigen comprises the amino acid sequence of amino acids 1 to 221 of SEQ ID NO: 29.

In one embodiment, RNA encoding a vaccine antigen (i) comprises the nucleotide sequence of nucleotides 54 to 716 of SEQ ID NO: 30, a nucleotide sequence having at least 99%, 98%, 97%, 96%, 95%, 90%, 85%, or 80% identity to the nucleotide sequence of nucleotides 54 to 716 of SEQ ID NO: 30, or a fragment of the nucleotide sequence of nucleotides 54 to 716 of SEQ ID NO: 30, or the nucleotide sequence having at least 99%, 98%, 97%, 96%, 95%, 90%, 85%, or 80% identity to the nucleotide sequence of nucleotides 54 to 716 of SEQ ID NO: 30; and/or (ii) encodes an amino acid sequence comprising the amino acid sequence of amino acids 1 to 221 of SEQ ID NO: 29, an amino acid sequence having at least 99%, 98%, 97%, 96%, 95%, 90%, 85%, or 80% identity to the amino acid sequence of amino acids 1 to 221 of SEQ ID NO: 29, or an immunogenic fragment of the amino acid sequence of amino acids 1 to 221 of SEQ ID NO: 29, or the amino acid sequence having at least 99%, 98%, 97%, 96%, 95%, 90%, 85%, or 80% identity to the amino acid sequence of amino acids 1 to 221 of SEQ ID NO: 29. In one embodiment, RNA encoding a vaccine antigen (i) comprises the nucleotide sequence of nucleotides 54 to 716 of SEQ ID NO: 30; and/or (ii) encodes an amino acid sequence comprising the amino acid sequence of amino acids 1 to 221 of SEQ ID NO: 29.

In one embodiment, a vaccine antigen comprises the amino acid sequence of amino acids 1 to 224 of SEQ ID NO: 31, an amino acid sequence having at least 99%, 98%, 97%, 96%, 95%, 90%, 85%, or 80% identity to the amino acid sequence of amino acids 1 to 224 of SEQ ID NO: 31, or an immunogenic fragment of the amino acid sequence of amino acids 1 to 224 of SEQ ID NO: 31, or the amino acid sequence having at least 99%, 98%, 97%, 96%, 95%, 90%, 85%, or 80% identity to the amino acid sequence of amino acids 1 to 224 of SEQ ID NO: 31. In one embodiment, a vaccine antigen comprises the amino acid sequence of amino acids 1 to 224 of SEQ ID NO: 31. In one embodiment, RNA encoding a vaccine antigen (i) comprises the nucleotide sequence of nucleotides 54 to 725 of SEQ ID NO: 32, a nucleotide sequence having at least 99%, 98%, 97%, 96%, 95%, 90%, 85%, or 80% identity to the nucleotide sequence of nucleotides 54 to 725 of SEQ ID NO: 32, or a fragment of the nucleotide sequence of nucleotides 54 to 725 of SEQ ID NO: 32, or the nucleotide sequence having at least 99%, 98%, 97%, 96%, 95%, 90%, 85%, or 80% identity to the nucleotide sequence of nucleotides 54 to 725 of SEQ ID NO: 32; and/or (ii) encodes an amino acid sequence comprising the amino acid sequence of amino acids 1 to 224 of SEQ ID NO: 31, an amino acid sequence having at least 99%, 98%, 97%, 96%, 95%, 90%, 85%, or 80% identity to the amino acid sequence of amino acids 1 to 224 of SEQ ID NO: 31, or an immunogenic fragment of the amino acid sequence of amino acids 1 to 224 of SEQ ID NO: 31, or the amino acid sequence having at least 99%, 98%, 97%, 96%, 95%, 90%, 85%, or 80% identity to the amino acid sequence of amino acids 1 to 224 of SEQ ID NO: 31. In one embodiment, RNA encoding a vaccine antigen (i) comprises the nucleotide sequence of nucleotides 54 to 725 of SEQ ID NO: 32; and/or (ii) encodes an amino acid sequence comprising the amino acid sequence of amino acids 1 to 224 of SEQ ID NO: 31.

According to certain embodiments, a trimerization domain is fused, either directly or through a linker, e.g., a glycine/serine linker, to a SARS-CoV-2 S protein, a variant thereof, or a fragment thereof, i.e., the antigenic peptide or protein. Accordingly, in one embodiment, a trimerization domain is fused to the above described amino acid sequences derived from SARS-CoV-2 S protein or immunogenic fragments thereof (antigenic peptides or proteins) comprised by the vaccine antigens described above (which may optionally be fused to a signal peptide as described above).

Such trimerization domains are preferably located at the C-terminus of the antigenic peptide or protein, without being limited thereto. Trimerization domains as defined herein preferably allow the trimerization of the antigenic peptide or protein as encoded by an RNA. Examples of trimerization domains as defined herein include, without being limited thereto, foldon, the natural trimerization domain of T4 fibritin. The C-terminal domain of T4 fibritin (foldon) is obligatory for the formation of the fibritin trimer structure and can be used as an artificial trimerization domain. In one embodiment, the trimerization domain as defined herein includes, without being limited thereto, a sequence comprising the amino acid sequence of amino acids 3 to 29 of SEQ ID NO: 10 or a functional variant thereof. In one embodiment, the trimerization domain as defined herein includes, without being limited thereto, a sequence comprising the amino acid sequence of SEQ ID NO: 10 or a functional variant thereof.

In one embodiment, a trimerization domain comprises the amino acid sequence of amino acids 3 to 29 of SEQ ID NO: 10, an amino acid sequence having at least 99%, 98%, 97%, 96%, 95%, 90%, 85%, or 80% identity to the amino acid sequence of amino acids 3 to 29 of SEQ ID NO: 10, or a functional fragment of the amino acid sequence of amino acids 3 to 29 of SEQ ID NO: 10, or the amino acid sequence having at least 99%, 98%, 97%, 96%, 95%, 90%, 85%, or 80% identity to the amino acid sequence of amino acids 3 to 29 of SEQ ID NO: 10. In one embodiment, a trimerization domain comprises the amino acid sequence of amino acids 3 to 29 of SEQ ID NO: 10. In one embodiment, RNA encoding a trimerization domain (i) comprises the nucleotide sequence of nucleotides 7 to 87 of SEQ ID NO: 11, a nucleotide sequence having at least 99%, 98%, 97%, 96%, 95%, 90%, 85%, or 80% identity to the nucleotide sequence of nucleotides 7 to 87 of SEQ ID NO: 11, or a fragment of the nucleotide sequence of nucleotides 7 to 87 of SEQ ID NO: 11, or the nucleotide sequence having at least 99%, 98%, 97%, 96%, 95%, 90%, 85%, or 80% identity to the nucleotide sequence of nucleotides 7 to 87 of SEQ ID NO: 11; and/or (ii) encodes an amino acid sequence comprising the amino acid sequence of amino acids 3 to 29 of SEQ ID NO: 10, an amino acid sequence having at least 99%, 98%, 97%, 96%, 95%, 90%, 85%, or 80% identity to the amino acid sequence of amino acids 3 to 29 of SEQ ID NO: 10, or a functional fragment of the amino acid sequence of amino acids 3 to 29 of SEQ ID NO: 10, or the amino acid sequence having at least 99%, 98%, 97%, 96%, 95%, 90%, 85%, or 80% identity to the amino acid sequence of amino acids 3 to 29 of SEQ ID NO: 10. In one embodiment, RNA encoding a trimerization domain (i) comprises the nucleotide sequence of nucleotides 7 to 87 of SEQ ID NO: 11; and/or (ii) encodes an amino acid sequence comprising the amino acid sequence of amino acids 3 to 29 of SEQ ID NO: 10.

In one embodiment, a trimerization domain comprises the amino acid sequence SEQ ID NO: 10, an amino acid sequence having at least 99%, 98%, 97%, 96%, 95%, 90%, 85%, or 80% identity to the amino acid sequence of SEQ ID NO: 10, or a functional fragment of the amino acid sequence of SEQ ID NO: 10, or the amino acid sequence having at least 99%, 98%, 97%, 96%, 95%, 90%, 85%, or 80% identity to the amino acid sequence of SEQ ID NO: 10. In one embodiment, a trimerization domain comprises the amino acid sequence of SEQ ID NO: 10.

In one embodiment, RNA encoding a trimerization domain (i) comprises the nucleotide sequence of SEQ ID NO: 11, a nucleotide sequence having at least 99%, 98%, 97%, 96%, 95%, 90%, 85%, or 80% identity to the nucleotide sequence of SEQ ID NO: 11, or a fragment of the nucleotide sequence of SEQ ID NO: 11, or the nucleotide sequence having at least 99%, 98%, 97%, 96%, 95%, 90%, 85%, or 80% identity to the nucleotide sequence of SEQ ID NO: 11; and/or (ii) encodes an amino acid sequence comprising the amino acid sequence of SEQ ID NO: 10, an amino acid sequence having at least 99%, 98%, 97%, 96%, 95%, 90%, 85%, or 80% identity to the amino acid sequence of SEQ ID NO: 10, or a functional fragment of the amino acid sequence of SEQ ID NO: 10, or the amino acid sequence having at least 99%, 98%, 97%, 96%, 95%, 90%, 85%, or 80% identity to the amino acid sequence of SEQ ID NO: 10. In one embodiment, RNA encoding a trimerization domain (i) comprises the nucleotide sequence of SEQ ID NO: 11; and/or (ii) encodes an amino acid sequence comprising the amino acid sequence of SEQ ID NO: 10.

Such trimerization domains are preferably used in order to promote trimerization of the encoded antigenic peptide or protein. More preferably, a trimerization domain as defined herein is fused to an antigenic peptide or protein as defined herein.

Accordingly, in particularly preferred embodiments, RNA described herein comprises at least one coding region encoding an antigenic peptide or protein and a trimerization domain as defined herein, said trimerization domain preferably being fused to the antigenic peptide or protein, more preferably to the C-terminus of the antigenic peptide or protein.

In one embodiment, a vaccine antigen comprises the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5. In one embodiment, a vaccine antigen comprises the amino acid sequence of SEQ ID NO: 5.

In one embodiment, RNA encoding a vaccine antigen (i) comprises the nucleotide sequence of SEQ ID NO: 6, a nucleotide sequence having at least 99%, 98%, 97%, 96%, 95%, 90%, 85%, or 80% identity to the nucleotide sequence of SEQ ID NO: 6, or a fragment of the nucleotide sequence of SEQ ID NO: 6, or the nucleotide sequence having at least 99%, 98%, 97%, 96%, 95%, 90%, 85%, or 80% identity to the nucleotide sequence of SEQ ID NO: 6; and/or (ii) encodes an amino acid sequence comprising the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5. In one embodiment, RNA encoding a vaccine antigen (i) comprises the nucleotide sequence of SEQ ID NO: 6; and/or (ii) encodes an amino acid sequence comprising the amino acid sequence of SEQ ID NO: 5.

In one embodiment, RNA encoding a vaccine antigen (i) comprises the nucleotide sequence of SEQ ID NO: 17, 21, or 26, a nucleotide sequence having at least 99%, 98%, 97%, 96%, 95%, 90%, 85%, or 80% identity to the nucleotide sequence of SEQ ID NO: 17, 21, or 26, or a fragment of the nucleotide sequence of SEQ ID NO: 17, 21, or 26, or the nucleotide sequence having at least 99%, 98%, 97%, 96%, 95%, 90%, 85%, or 80% identity to the nucleotide sequence of SEQ ID NO: 17, 21, or 26; and/or (ii) encodes an amino acid sequence comprising the amino acid sequence of SEQ ID NO: 5, an amino acid sequence having at least 99%, 98%, 97%, 96%, 95%, 90%, 85%, or 80% identity to the amino acid sequence of SEQ ID NO: 5, or an immunogenic fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5. In one embodiment, RNA encoding a vaccine antigen (i) comprises the nucleotide sequence of SEQ ID NO: 17, 21, or 26; and/or (ii) encodes an amino acid sequence comprising the amino acid sequence of SEQ ID NO: 5.

In one embodiment, a vaccine antigen comprises the amino acid sequence of SEQ ID NO: 18, an amino acid sequence having at least 99%, 98%, 97%, 96%, 95%, 90%, 85%, or 80% identity to the amino acid sequence of SEQ ID NO: 18, or an immunogenic fragment of the amino acid sequence of SEQ ID NO: 18, or the amino acid sequence having at least 99%, 98%, 97%, 96%, 95%, 90%, 85%, or 80% identity to the amino acid sequence of SEQ ID NO: 18. In one embodiment, a vaccine antigen comprises the amino acid sequence of SEQ ID NO: 18.

In one embodiment, a vaccine antigen comprises the amino acid sequence of amino acids 1 to 257 of SEQ ID NO: 29, an amino acid sequence having at least 99%, 98%, 97%, 96%, 95%, 90%, 85%, or 80% identity to the amino acid sequence of amino acids 1 to 257 of SEQ ID NO: 29, or an immunogenic fragment of the amino acid sequence of amino acids 1 to 257 of SEQ ID NO: 29, or the amino acid sequence having at least 99%, 98%, 97%, 96%, 95%, 90%, 85%, or 80% identity to the amino acid sequence of amino acids 1 to 257 of SEQ ID NO: 29. In one embodiment, a vaccine antigen comprises the amino acid sequence of amino acids 1 to 257 of SEQ ID NO: 29.

In one embodiment, RNA encoding a vaccine antigen (i) comprises the nucleotide sequence of nucleotides 54 to 824 of SEQ ID NO: 30, a nucleotide sequence having at least 99%, 98%, 97%, 96%, 95%, 90%, 85%, or 80% identity to the nucleotide sequence of nucleotides 54 to 824 of SEQ ID NO: 30, or a fragment of the nucleotide sequence of nucleotides 54 to 824 of SEQ ID NO: 30, or the nucleotide sequence having at least 99%, 98%, 97%, 96%, 95%, 90%, 85%, or 80% identity to the nucleotide sequence of nucleotides 54 to 824 of SEQ ID NO: 30; and/or (ii) encodes an amino acid sequence comprising the amino acid sequence of amino acids 1 to 257 of SEQ ID NO: 29, an amino acid sequence having at least 99%, 98%, 97%, 96%, 95%, 90%, 85%, or 80% identity to the amino acid sequence of amino acids 1 to 257 of SEQ ID NO: 29, or an immunogenic fragment of the amino acid sequence of amino acids 1 to 257 of SEQ ID NO: 29, or the amino acid sequence having at least 99%, 98%, 97%, 96%, 95%, 90%, 85%, or 80% identity to the amino acid sequence of amino acids 1 to 257 of SEQ ID NO: 29. In one embodiment, RNA encoding a vaccine antigen (i) comprises the nucleotide sequence of nucleotides 54 to 824 of SEQ ID NO: 30; and/or (ii) encodes an amino acid sequence comprising the amino acid sequence of amino acids 1 to 257 of SEQ ID NO: 29.

In one embodiment, a vaccine antigen comprises the amino acid sequence of amino acids 1 to 260 of SEQ ID NO: 31, an amino acid sequence having at least 99%, 98%, 97%, 96%, 95%, 90%, 85%, or 80% identity to the amino acid sequence of amino acids 1 to 260 of SEQ ID NO: 31, or an immunogenic fragment of the amino acid sequence of amino acids 1 to 260 of SEQ ID NO: 31, or the amino acid sequence having at least 99%, 98%, 97%, 96%, 95%, 90%, 85%, or 80% identity to the amino acid sequence of amino acids 1 to 260 of SEQ ID NO: 31. In one embodiment, a vaccine antigen comprises the amino acid sequence of amino acids 1 to 260 of SEQ ID NO: 31.

In one embodiment, RNA encoding a vaccine antigen (i) comprises the nucleotide sequence of nucleotides 54 to 833 of SEQ ID NO: 32, a nucleotide sequence having at least 99%, 98%, 97%, 96%, 95%, 90%, 85%, or 80% identity to the nucleotide sequence of nucleotides 54 to 833 of SEQ ID NO: 32, or a fragment of the nucleotide sequence of nucleotides 54 to 833 of SEQ ID NO: 32, or the nucleotide sequence having at least 99%, 98%, 97%, 96%, 95%, 90%, 85%, or 80% identity to the nucleotide sequence of nucleotides 54 to 833 of SEQ ID NO: 32; and/or (ii) encodes an amino acid sequence comprising the amino acid sequence of amino acids 1 to 260 of SEQ ID NO: 31, an amino acid sequence having at least 99%, 98%, 97%, 96%, 95%, 90%, 85%, or 80% identity to the amino acid sequence of amino acids 1 to 260 of SEQ ID NO: 31, or an immunogenic fragment of the amino acid sequence of amino acids 1 to 260 of SEQ ID NO: 31, or the amino acid sequence having at least 99%, 98%, 97%, 96%, 95%, 90%, 85%, or 80% identity to the amino acid sequence of amino acids 1 to 260 of SEQ ID NO: 31. In one embodiment, RNA encoding a vaccine antigen (i) comprises the nucleotide sequence of nucleotides 54 to 833 of SEQ ID NO: 32; and/or (ii) encodes an amino acid sequence comprising the amino acid sequence of amino acids 1 to 260 of SEQ ID NO: 31.

In one embodiment, a vaccine antigen comprises the amino acid sequence of amino acids 20 to 257 of SEQ ID NO: 29, an amino acid sequence having at least 99%, 98%, 97%, 96%, 95%, 90%, 85%, or 80% identity to the amino acid sequence of amino acids 20 to 257 of SEQ ID NO: 29, or an immunogenic fragment of the amino acid sequence of amino acids 20 to 257 of SEQ ID NO: 29, or the amino acid sequence having at least 99%, 98%, 97%, 96%, 95%, 90%, 85%, or 80% identity to the amino acid sequence of amino acids 20 to 257 of SEQ ID NO: 29. In one embodiment, a vaccine antigen comprises the amino acid sequence of amino acids 20 to 257 of SEQ ID NO: 29.

In one embodiment, RNA encoding a vaccine antigen (i) comprises the nucleotide sequence of nucleotides 111 to 824 of SEQ ID NO: 30, a nucleotide sequence having at least 99%, 98%, 97%, 96%, 95%, 90%, 85%, or 80% identity to the nucleotide sequence of nucleotides 111 to 824 of SEQ ID NO: 30, or a fragment of the nucleotide sequence of nucleotides 111 to 824 of SEQ ID NO: 30, or the nucleotide sequence having at least 99%, 98%, 97%, 96%, 95%, 90%, 85%, or 80% identity to the nucleotide sequence of nucleotides 111 to 824 of SEQ ID NO: 30; and/or (ii) encodes an amino acid sequence comprising the amino acid sequence of amino acids 20 to 257 of SEQ ID NO: 29, an amino acid sequence having at least 99%, 98%, 97%, 96%, 95%, 90%, 85%, or 80% identity to the amino acid sequence of amino acids 20 to 257 of SEQ ID NO: 29, or an immunogenic fragment of the amino acid sequence of amino acids 20 to 257 of SEQ ID NO: 29, or the amino acid sequence having at least 99%, 98%, 97%, 96%, 95%, 90%, 85%, or 80% identity to the amino acid sequence of amino acids 20 to 257 of SEQ ID NO: 29. In one embodiment, RNA encoding a vaccine antigen (i) comprises the nucleotide sequence of nucleotides 111 to 824 of SEQ ID NO: 30; and/or (ii) encodes an amino acid sequence comprising the amino acid sequence of amino acids 20 to 257 of SEQ ID NO: 29.

In one embodiment, a vaccine antigen comprises the amino acid sequence of amino acids 23 to 260 of SEQ ID NO: 31, an amino acid sequence having at least 99%, 98%, 97%, 96%, 95%, 90%, 85%, or 80% identity to the amino acid sequence of amino acids 23 to 260 of SEQ ID NO: 31, or an immunogenic fragment of the amino acid sequence of amino acids 23 to 260 of SEQ ID NO: 31, or the amino acid sequence having at least 99%, 98%, 97%, 96%, 95%, 90%, 85%, or 80% identity to the amino acid sequence of amino acids 23 to 260 of SEQ ID NO: 31. In one embodiment, a vaccine antigen comprises the amino acid sequence of amino acids 23 to 260 of SEQ ID NO: 31.

In one embodiment, RNA encoding a vaccine antigen (i) comprises the nucleotide sequence of nucleotides 120 to 833 of SEQ ID NO: 32, a nucleotide sequence having at least 99%, 98%, 97%, 96%, 95%, 90%, 85%, or 80% identity to the nucleotide sequence of nucleotides 120 to 833 of SEQ ID NO: 32, or a fragment of the nucleotide sequence of nucleotides 120 to 833 of SEQ ID NO: 32, or the nucleotide sequence having at least 99%, 98%, 97%, 96%, 95%, 90%, 85%, or 80% identity to the nucleotide sequence of nucleotides 120 to 833 of SEQ ID NO: 32; and/or (ii) encodes an amino acid sequence comprising the amino acid sequence of amino acids 23 to 260 of SEQ ID NO: 31, an amino acid sequence having at least 99%, 98%, 97%, 96%, 95%, 90%, 85%, or 80% identity to the amino acid sequence of amino acids 23 to 260 of SEQ ID NO: 31, or an immunogenic fragment of the amino acid sequence of amino acids 23 to 260 of SEQ ID NO: 31, or the amino acid sequence having at least 99%, 98%, 97%, 96%, 95%, 90%, 85%, or 80% identity to the amino acid sequence of amino acids 23 to 260 of SEQ ID NO: 31. In one embodiment, RNA encoding a vaccine antigen (i) comprises the nucleotide sequence of nucleotides 120 to 833 of SEQ ID NO: 32; and/or (ii) encodes an amino acid sequence comprising the amino acid sequence of amino acids 23 to 260 of SEQ ID NO: 31.

According to certain embodiments, a transmembrane domain is fused, either directly or through a linker, e.g., a glycine/serine linker, to a SARS-CoV-2 S protein, a variant thereof, or a fragment thereof, i.e., the antigenic peptide or protein. Accordingly, in one embodiment, a transmembrane domain is fused to the above described amino acid sequences derived from SARS-CoV-2 S protein or immunogenic fragments thereof (antigenic peptides or proteins) comprised by the vaccine antigens described above (which may optionally be fused to a signal peptide and/or trimerization domain as described above).

Such transmembrane domains are preferably located at the C-terminus of the antigenic peptide or protein, without being limited thereto. Preferably, such transmembrane domains are located at the C-terminus of the trimerization domain, if present, without being limited thereto. In one embodiment, a trimerization domain is present between the SARS-CoV-2 S protein, a variant thereof, or a fragment thereof, i.e., the antigenic peptide or protein, and the transmembrane domain.

Transmembrane domains as defined herein preferably allow the anchoring into a cellular membrane of the antigenic peptide or protein as encoded by an RNA.

In one embodiment, the transmembrane domain sequence as defined herein includes, without being limited thereto, the transmembrane domain sequence of SARS-CoV-2 S protein, in particular a sequence comprising the amino acid sequence of amino acids 1207 to 1254 of SEQ ID NO: 1 or a functional variant thereof.

In one embodiment, a transmembrane domain sequence comprises the amino acid sequence of amino acids 1207 to 1254 of SEQ ID NO: 1, an amino acid sequence having at least 99%, 98%, 97%, 96%, 95%, 90%, 85%, or 80% identity to the amino acid sequence of amino acids 1207 to 1254 of SEQ ID NO: 1, or a functional fragment of the amino acid sequence of amino acids 1207 to 1254 of SEQ ID NO: 1, or the amino acid sequence having at least 99%, 98%, 97%, 96%, 95%, 90%, 85%, or 80% identity to the amino acid sequence of amino acids 1207 to 1254 of SEQ ID NO: 1. In one embodiment, a transmembrane domain sequence comprises the amino acid sequence of amino acids 1207 to 1254 of SEQ ID NO: 1.

In one embodiment, RNA encoding a transmembrane domain sequence (i) comprises the nucleotide sequence of nucleotides 3619 to 3762 of SEQ ID NO: 2, 8 or 9, a nucleotide sequence having at least 99%, 98%, 97%, 96%, 95%, 90%, 85%, or 80% identity to the nucleotide sequence of nucleotides 3619 to 3762 of SEQ ID NO: 2, 8 or 9, or a fragment of the nucleotide sequence of nucleotides 3619 to 3762 of SEQ ID NO: 2, 8 or 9, or the nucleotide sequence having at least 99%, 98%, 97%, 96%, 95%, 90%, 85%, or 80% identity to the nucleotide sequence of nucleotides 3619 to 3762 of SEQ ID NO: 2, 8 or 9; and/or (ii) encodes an amino acid sequence comprising the amino acid sequence of amino acids 1207 to 1254 of SEQ ID NO: 1, an amino acid sequence having at least 99%, 98%, 97%, 96%, 95%, 90%, 85%, or 80% identity to the amino acid sequence of amino acids 1207 to 1254 of SEQ ID NO: 1, or a functional fragment of the amino acid sequence of amino acids 1207 to 1254 of SEQ ID NO: 1, or the amino acid sequence having at least 99%, 98%, 97%, 96%, 95%, 90%, 85%, or 80% identity to the amino acid sequence of amino acids 1207 to 1254 of SEQ ID NO: 1. In one embodiment, RNA encoding a transmembrane domain sequence (i) comprises the nucleotide sequence of nucleotides 3619 to 3762 of SEQ ID NO: 2, 8 or 9; and/or (ii) encodes an amino acid sequence comprising the amino acid sequence of amino acids 1207 to 1254 of SEQ ID NO: 1.

In one embodiment, a vaccine antigen comprises the amino acid sequence of amino acids 1 to 311 of SEQ ID NO: 29, an amino acid sequence having at least 99%, 98%, 97%, 96%, 95%, 90%, 85%, or 80% identity to the amino acid sequence of amino acids 1 to 311 of SEQ ID NO: 29, or an immunogenic fragment of the amino acid sequence of amino acids 1 to 311 of SEQ ID NO: 29, or the amino acid sequence having at least 99%, 98%, 97%, 96%, 95%, 90%, 85%, or 80% identity to the amino acid sequence of amino acids 1 to 311 of SEQ ID NO: 29. In one embodiment, a vaccine antigen comprises the amino acid sequence of amino acids 1 to 311 of SEQ ID NO: 29.

In one embodiment, RNA encoding a vaccine antigen (i) comprises the nucleotide sequence of nucleotides 54 to 986 of SEQ ID NO: 30, a nucleotide sequence having at least 99%, 98%, 97%, 96%, 95%, 90%, 85%, or 80% identity to the nucleotide sequence of nucleotides 54 to 986 of SEQ ID NO: 30, or a fragment of the nucleotide sequence of nucleotides 54 to 986 of SEQ ID NO: 30, or the nucleotide sequence having at least 99%, 98%, 97%, 96%, 95%, 90%, 85%, or 80% identity to the nucleotide sequence of nucleotides 54 to 986 of SEQ ID NO: 30; and/or (ii) encodes an amino acid sequence comprising the amino acid sequence of amino acids 1 to 311 of SEQ ID NO: 29, an amino acid sequence having at least 99%, 98%, 97%, 96%, 95%, 90%, 85%, or 80% identity to the amino acid sequence of amino acids 1 to 311 of SEQ ID NO: 29, or an immunogenic fragment of the amino acid sequence of amino acids 1 to 311 of SEQ ID NO: 29, or the amino acid sequence having at least 99%, 98%, 97%, 96%, 95%, 90%, 85%, or 80% identity to the amino acid sequence of amino acids 1 to 311 of SEQ ID NO: 29. In one embodiment, RNA encoding a vaccine antigen (i) comprises the nucleotide sequence of nucleotides 54 to 986 of SEQ ID NO: 30; and/or (ii) encodes an amino acid sequence comprising the amino acid sequence of amino acids 1 to 311 of SEQ ID NO: 29.

In one embodiment, a vaccine antigen comprises the amino acid sequence of amino acids 1 to 314 of SEQ ID NO: 31, an amino acid sequence having at least 99%, 98%, 97%, 96%, 95%, 90%, 85%, or 80% identity to the amino acid sequence of amino acids 1 to 314 of SEQ ID NO: 31, or an immunogenic fragment of the amino acid sequence of amino acids 1 to 314 of SEQ ID NO: 31, or the amino acid sequence having at least 99%, 98%, 97%, 96%, 95%, 90%, 85%, or 80% identity to the amino acid sequence of amino acids 1 to 314 of SEQ ID NO: 31. In one embodiment, a vaccine antigen comprises the amino acid sequence of amino acids 1 to 314 of SEQ ID NO: 31.

In one embodiment, RNA encoding a vaccine antigen (i) comprises the nucleotide sequence of nucleotides 54 to 995 of SEQ ID NO: 32, a nucleotide sequence having at least 99%, 98%, 97%, 96%, 95%, 90%, 85%, or 80% identity to the nucleotide sequence of nucleotides 54 to 995 of SEQ ID NO: 32, or a fragment of the nucleotide sequence of nucleotides 54 to 995 of SEQ ID NO: 32, or the nucleotide sequence having at least 99%, 98%, 97%, 96%, 95%, 90%, 85%, or 80% identity to the nucleotide sequence of nucleotides 54 to 995 of SEQ ID NO: 32; and/or (ii) encodes an amino acid sequence comprising the amino acid sequence of amino acids 1 to 314 of SEQ ID NO: 31, an amino acid sequence having at least 99%, 98%, 97%, 96%, 95%, 90%, 85%, or 80% identity to the amino acid sequence of amino acids 1 to 314 of SEQ ID NO: 31, or an immunogenic fragment of the amino acid sequence of amino acids 1 to 314 of SEQ ID NO: 31, or the amino acid sequence having at least 99%, 98%, 97%, 96%, 95%, 90%, 85%, or 80% identity to the amino acid sequence of amino acids 1 to 314 of SEQ ID NO: 31. In one embodiment, RNA encoding a vaccine antigen (i) comprises the nucleotide sequence of nucleotides 54 to 995 of SEQ ID NO: 32; and/or (ii) encodes an amino acid sequence comprising the amino acid sequence of amino acids 1 to 314 of SEQ ID NO: 31.

In one embodiment, a vaccine antigen comprises the amino acid sequence of amino acids 20 to 311 of SEQ ID NO: 29, an amino acid sequence having at least 99%, 98%, 97%, 96%, 95%, 90%, 85%, or 80% identity to the amino acid sequence of amino acids 20 to 311 of SEQ ID NO: 29, or an immunogenic fragment of the amino acid sequence of amino acids 20 to 311 of SEQ ID NO: 29, or the amino acid sequence having at least 99%, 98%, 97%, 96%, 95%, 90%, 85%, or 80% identity to the amino acid sequence of amino acids 20 to 311 of SEQ ID NO: 29. In one embodiment, a vaccine antigen comprises the amino acid sequence of amino acids 20 to 311 of SEQ ID NO: 29.

In one embodiment, RNA encoding a vaccine antigen (i) comprises the nucleotide sequence of nucleotides 111 to 986 of SEQ ID NO: 30, a nucleotide sequence having at least 99%, 98%, 97%, 96%, 95%, 90%, 85%, or 80% identity to the nucleotide sequence of nucleotides 111 to 986 of SEQ ID NO: 30, or a fragment of the nucleotide sequence of nucleotides 111 to 986 of SEQ ID NO: 30, or the nucleotide sequence having at least 99%, 98%, 97%, 96%, 95%, 90%, 85%, or 80% identity to the nucleotide sequence of nucleotides 111 to 986 of SEQ ID NO: 30; and/or (ii) encodes an amino acid sequence comprising the amino acid sequence of amino acids 20 to 311 of SEQ ID NO: 29, an amino acid sequence having at least 99%, 98%, 97%, 96%, 95%, 90%, 85%, or 80% identity to the amino acid sequence of amino acids 20 to 311 of SEQ ID NO: 29, or an immunogenic fragment of the amino acid sequence of amino acids 20 to 311 of SEQ ID NO: 29, or the amino acid sequence having at least 99%, 98%, 97%, 96%, 95%, 90%, 85%, or 80% identity to the amino acid sequence of amino acids 20 to 311 of SEQ ID NO: 29. In one embodiment, RNA encoding a vaccine antigen (i) comprises the nucleotide sequence of nucleotides 111 to 986 of SEQ ID NO: 30; and/or (ii) encodes an amino acid sequence comprising the amino acid sequence of amino acids 20 to 311 of SEQ ID NO: 29.

In one embodiment, a vaccine antigen comprises the amino acid sequence of amino acids 23 to 314 of SEQ ID NO: 31, an amino acid sequence having at least 99%, 98%, 97%, 96%, 95%, 90%, 85%, or 80% identity to the amino acid sequence of amino acids 23 to 314 of SEQ ID NO: 31, or an immunogenic fragment of the amino acid sequence of amino acids 23 to 314 of SEQ ID NO: 31, or the amino acid sequence having at least 99%, 98%, 97%, 96%, 95%, 90%, 85%, or 80% identity to the amino acid sequence of amino acids 23 to 314 of SEQ ID NO: 31. In one embodiment, a vaccine antigen comprises the amino acid sequence of amino acids 23 to 314 of SEQ ID NO: 31.

In one embodiment, RNA encoding a vaccine antigen (i) comprises the nucleotide sequence of nucleotides 120 to 995 of SEQ ID NO: 32, a nucleotide sequence having at least 99%, 98%, 97%, 96%, 95%, 90%, 85%, or 80% identity to the nucleotide sequence of nucleotides 120 to 995 of SEQ ID NO: 32, or a fragment of the nucleotide sequence of nucleotides 120 to 995 of SEQ ID NO: 32, or the nucleotide sequence having at least 99%, 98%, 97%, 96%, 95%, 90%, 85%, or 80% identity to the nucleotide sequence of nucleotides 120 to 995 of SEQ ID NO: 32; and/or (ii) encodes an amino acid sequence comprising the amino acid sequence of amino acids 23 to 314 of SEQ ID NO: 31, an amino acid sequence having at least 99%, 98%, 97%, 96%, 95%, 90%, 85%, or 80% identity to the amino acid sequence of amino acids 23 to 314 of SEQ ID NO: 31, or an immunogenic fragment of the amino acid sequence of amino acids 23 to 314 of SEQ ID NO: 31, or the amino acid sequence having at least 99%, 98%, 97%, 96%, 95%, 90%, 85%, or 80% identity to the amino acid sequence of amino acids 23 to 314 of SEQ ID NO: 31. In one embodiment, RNA encoding a vaccine antigen (i) comprises the nucleotide sequence of nucleotides 120 to 995 of SEQ ID NO: 32; and/or (ii) encodes an amino acid sequence comprising the amino acid sequence of amino acids 23 to 314 of SEQ ID NO: 31.

In one embodiment, RNA encoding a vaccine antigen (i) comprises the nucleotide sequence of SEQ ID NO: 30, a nucleotide sequence having at least 99%, 98%, 97%, 96%, 95%, 90%, 85%, or 80% identity to the nucleotide sequence of SEQ ID NO: 30, or a fragment of the nucleotide sequence of SEQ ID NO: 30, or the nucleotide sequence having at least 99%, 98%, 97%, 96%, 95%, 90%, 85%, or 80% identity to the nucleotide sequence of SEQ ID NO: 30; and/or (ii) encodes an amino acid sequence comprising the amino acid sequence of SEQ ID NO: 29, an amino acid sequence having at least 99%, 98%, 97%, 96%, 95%, 90%, 85%, or 80% identity to the amino acid sequence of SEQ ID NO: 29, or an immunogenic fragment of the amino acid sequence of SEQ ID NO: 29, or the amino acid sequence having at least 99%, 98%, 97%, 96%, 95%, 90%, 85%, or 80% identity to the amino acid sequence of SEQ ID NO: 29. In one embodiment, RNA encoding a vaccine antigen (i) comprises the nucleotide sequence of SEQ ID NO: 30; and/or (ii) encodes an amino acid sequence comprising the amino acid sequence of SEQ ID NO: 29.

In one embodiment, RNA encoding a vaccine antigen (i) comprises the nucleotide sequence of SEQ ID NO: 32, a nucleotide sequence having at least 99%, 98%, 97%, 96%, 95%, 90%, 85%, or 80% identity to the nucleotide sequence of SEQ ID NO: 32, or a fragment of the nucleotide sequence of SEQ ID NO: 32, or the nucleotide sequence having at least 99%, 98%, 97%, 96%, 95%, 90%, 85%, or 80% identity to the nucleotide sequence of SEQ ID NO: 32; and/or (ii) encodes an amino acid sequence comprising the amino acid sequence of SEQ ID NO: 31, an amino acid sequence having at least 99%, 98%, 97%, 96%, 95%, 90%, 85%, or 80% identity to the amino acid sequence of SEQ ID NO: 31, or an immunogenic fragment of the amino acid sequence of SEQ ID NO: 31, or the amino acid sequence having at least 99%, 98%, 97%, 96%, 95%, 90%, 85%, or 80% identity to the amino acid sequence of SEQ ID NO: 31. In one embodiment, RNA encoding a vaccine antigen (i) comprises the nucleotide sequence of SEQ ID NO: 32; and/or (ii) encodes an amino acid sequence comprising the amino acid sequence of SEQ ID NO: 31.

In one embodiment, a vaccine antigen comprises the amino acid sequence of SEQ ID NO: 28, an amino acid sequence having at least 99%, 98%, 97%, 96%, 95%, 90%, 85%, or 80% identity to the amino acid sequence of SEQ ID NO: 28, or an immunogenic fragment of the amino acid sequence of SEQ ID NO: 28, or the amino acid sequence having at least 99%, 98%, 97%, 96%, 95%, 90%, 85%, or 80% identity to the amino acid sequence of SEQ ID NO: 28. In one embodiment, a vaccine antigen comprises the amino acid sequence of SEQ ID NO: 28.

In one embodiment, RNA encoding a vaccine antigen (i) comprises the nucleotide sequence of SEQ ID NO: 27, a nucleotide sequence having at least 99%, 98%, 97%, 96%, 95%, 90%, 85%, or 80% identity to the nucleotide sequence of SEQ ID NO: 27, or a fragment of the nucleotide sequence of SEQ ID NO: 27, or the nucleotide sequence having at least 99%, 98%, 97%, 96%, 95%, 90%, 85%, or 80% identity to the nucleotide sequence of SEQ ID NO: 27; and/or (ii) encodes an amino acid sequence comprising the amino acid sequence of SEQ ID NO: 28, an amino acid sequence having at least 99%, 98%, 97%, 96%, 95%, 90%, 85%, or 80% identity to the amino acid sequence of SEQ ID NO: 28, or an immunogenic fragment of the amino acid sequence of SEQ ID NO: 28, or the amino acid sequence having at least 99%, 98%, 97%, 96%, 95%, 90%, 85%, or 80% identity to the amino acid sequence of SEQ ID NO: 28. In one embodiment, RNA encoding a vaccine antigen (i) comprises the nucleotide sequence of SEQ ID NO: 27; and/or (ii) encodes an amino acid sequence comprising the amino acid sequence of SEQ ID NO: 28.

In one embodiment, a vaccine antigen comprises the amino acid sequence of SEQ ID NO: 49, an amino acid sequence having at least 99.5%, 99%, 98.5%, 98%, 98.5% or 97% identity to the amino acid sequence of SEQ ID NO: 49, or an immunogenic fragment of the amino acid sequence of SEQ ID NO: 49, or the amino acid sequence having at least 99.5%, 99%, 98.5%, 98%, 98.5% or 97% identity to the amino acid sequence of SEQ ID NO: 49.

In one embodiment, a vaccine antigen comprises the amino acid sequence of SEQ ID NO: 49. The amino acid sequence of SEQ ID NO: 49 corresponds to the amino acid sequence of the full-length S protein from Omicron BA.1, which includes proline residues at positions 986 and 987 of SEQ ID NO: 49.

In one embodiment, RNA encoding a vaccine antigen (i) comprises the nucleotide sequence of SEQ ID NO: 50, a nucleotide sequence having at least 99.5%, 99%, 98.5%, 98%, 98.5% or 97% identity to the nucleotide sequence of SEQ ID NO: 50, or a fragment of the nucleotide sequence of SEQ ID NO: 50, or the nucleotide sequence having at least 99.5%, 99%, 98.5%, 98%, 98.5% or 97% identity to the nucleotide sequence of SEQ ID NO: 50; and/or (ii) encodes an amino acid sequence comprising the amino acid sequence of SEQ ID NO: 49, an amino acid sequence having at least 99.5%, 99%, 98.5%, 98%, 98.5% or 97% identity to the amino acid sequence of SEQ ID NO: 49, or an immunogenic fragment of the amino acid sequence of SEQ ID NO: 49, or the amino acid sequence having at least 99.5%, 99%, 98.5%, 98%, 98.5% or 97% identity to the amino acid sequence of SEQ ID NO: 49. In one embodiment, RNA encoding a vaccine antigen (i) comprises the nucleotide sequence of SEQ ID NO: 50; and/or (ii) encodes an amino acid sequence comprising the amino acid sequence of SEQ ID NO: 49. The nucleotide sequence of SEQ ID NO: 50 is a nucleotide sequence designed to encode the amino acid sequence of the full-length S protein from Omicron BA.1 with proline residues at positions 986 and 987 of SEQ ID NO: 49.

In one embodiment, RNA encoding a vaccine antigen (i) comprises the nucleotide sequence of SEQ ID NO: 51, a nucleotide sequence having at least 99.5%, 99%, 98.5%, 98%, 98.5% or 97% identity to the nucleotide sequence of SEQ ID NO: 51, or a fragment of the nucleotide sequence of SEQ ID NO: 51, or the nucleotide sequence having at least 99.5%, 99%, 98.5%, 98%, 98.5% or 97% identity to the nucleotide sequence of SEQ ID NO: 51; and/or (ii) encodes an amino acid sequence comprising the amino acid sequence of SEQ ID NO: 49, an amino acid sequence having at least 99.5%, 99%, 98.5%, 98%, 98.5% or 97% identity to the amino acid sequence of SEQ ID NO: 49, or an immunogenic fragment of the amino acid sequence of SEQ ID NO: 49, or the amino acid sequence having at least 99.5%, 99%, 98.5%, 98%, 98.5% or 97% identity to the amino acid sequence of SEQ ID NO: 49. In one embodiment, RNA encoding a vaccine antigen (i) comprises the nucleotide sequence of SEQ ID NO: 51; and/or (ii) encodes an amino acid sequence comprising the amino acid sequence of SEQ ID NO: 49. The nucleotide sequence of SEQ ID NO: 51 corresponds to an RNA construct (e.g., comprising a 5′ UTR, a S-protein-encoding sequence, a 3′ UTR, and a poly-A tail), which encodes the amino acid sequence of the full-length S protein from Omicron BA.1 with proline residues at positions 986 and 987 of SEQ ID NO: 49.

In one embodiment, a vaccine antigen comprises the amino acid sequence of SEQ ID NO: 55, an amino acid sequence having at least 99.5%, 99%, 98.5%, 98%, 98.5% or 97% identity to the amino acid sequence of SEQ ID NO: 55, or an immunogenic fragment of the amino acid sequence of SEQ ID NO: 55, or the amino acid sequence having at least 99.5%, 99%, 98.5%, 98%, 98.5% or 97% identity to the amino acid sequence of SEQ ID NO: 55.

In one embodiment, a vaccine antigen comprises the amino acid sequence of SEQ ID NO: 55.

In one embodiment, RNA encoding a vaccine antigen (i) comprises the nucleotide sequence of SEQ ID NO: 56, a nucleotide sequence having at least 99.5%, 99%, 98.5%, 98%, 98.5% or 97% identity to the nucleotide sequence of SEQ ID NO: 56, or a fragment of the nucleotide sequence of SEQ ID NO: 56, or the nucleotide sequence having at least 99.5%, 99%, 98.5%, 98%, 98.5% or 97% identity to the nucleotide sequence of SEQ ID NO: 56; and/or (ii) encodes an amino acid sequence comprising the amino acid sequence of SEQ ID NO: 55, an amino acid sequence having at least 99.5%, 99%, 98.5%, 98%, 98.5% or 97% identity to the amino acid sequence of SEQ ID NO: 55, or an immunogenic fragment of the amino acid sequence of SEQ ID NO: 55, or the amino acid sequence having at least 99.5%, 99%, 98.5%, 98%, 98.5% or 97% identity to the amino acid sequence of SEQ ID NO: 55. In one embodiment, RNA encoding a vaccine antigen (i) comprises the nucleotide sequence of SEQ ID NO: 56; and/or (ii) encodes an amino acid sequence comprising the amino acid sequence of SEQ ID NO: 55.

In one embodiment, RNA encoding a vaccine antigen (i) comprises the nucleotide sequence of SEQ ID NO: 57, a nucleotide sequence having at least 99.5%, 99%, 98.5%, 98%, 98.5% or 97% identity to the nucleotide sequence of SEQ ID NO: 57, or a fragment of the nucleotide sequence of SEQ ID NO: 57, or the nucleotide sequence having at least 99.5%, 99%, 98.5%, 98%, 98.5% or 97% identity to the nucleotide sequence of SEQ ID NO: 57; and/or (ii) encodes an amino acid sequence comprising the amino acid sequence of SEQ ID NO: 55, an amino acid sequence having at least 99.5%, 99%, 98.5%, 98%, 98.5% or 97% identity to the amino acid sequence of SEQ ID NO: 55, or an immunogenic fragment of the amino acid sequence of SEQ ID NO: 55, or the amino acid sequence having at least 99.5%, 99%, 98.5%, 98%, 98.5% or 97% identity to the amino acid sequence of SEQ ID NO: 55. In one embodiment, RNA encoding a vaccine antigen (i) comprises the nucleotide sequence of SEQ ID NO: 57; and/or (ii) encodes an amino acid sequence comprising the amino acid sequence of SEQ ID NO: 55.

In one embodiment, a vaccine antigen comprises the amino acid sequence of SEQ ID NO: 58, an amino acid sequence having at least 99.5%, 99%, 98.5%, 98%, 98.5% or 97% identity to the amino acid sequence of SEQ ID NO: 58, or an immunogenic fragment of the amino acid sequence of SEQ ID NO: 58, or the amino acid sequence having at least 99.5%, 99%, 98.5%, 98%, 98.5% or 97% identity to the amino acid sequence of SEQ ID NO: 58.

In one embodiment, a vaccine antigen comprises the amino acid sequence of SEQ ID NO: 58.

In one embodiment, RNA encoding a vaccine antigen (i) comprises the nucleotide sequence of SEQ ID NO: 59, a nucleotide sequence having at least 99.5%, 99%, 98.5%, 98%, 98.5% or 97% identity to the nucleotide sequence of SEQ ID NO: 59, or a fragment of the nucleotide sequence of SEQ ID NO: 59, or the nucleotide sequence having at least 99.5%, 99%, 98.5%, 98%, 98.5% or 97% identity to the nucleotide sequence of SEQ ID NO: 59; and/or (ii) encodes an amino acid sequence comprising the amino acid sequence of SEQ ID NO: 58, an amino acid sequence having at least 99.5%, 99%, 98.5%, 98%, 98.5% or 97% identity to the amino acid sequence of SEQ ID NO: 58, or an immunogenic fragment of the amino acid sequence of SEQ ID NO: 58, or the amino acid sequence having at least 99.5%, 99%, 98.5%, 98%, 98.5% or 97% identity to the amino acid sequence of SEQ ID NO: 58. In one embodiment, RNA encoding a vaccine antigen (i) comprises the nucleotide sequence of SEQ ID NO: 59; and/or (ii) encodes an amino acid sequence comprising the amino acid sequence of SEQ ID NO: 58.

In one embodiment, RNA encoding a vaccine antigen (i) comprises the nucleotide sequence of SEQ ID NO: 60, a nucleotide sequence having at least 99.5%, 99%, 98.5%, 98%, 98.5% or 97% identity to the nucleotide sequence of SEQ ID NO: 60, or a fragment of the nucleotide sequence of SEQ ID NO: 60, or the nucleotide sequence having at least 99.5%, 99%, 98.5%, 98%, 98.5% or 97% identity to the nucleotide sequence of SEQ ID NO: 60; and/or (ii) encodes an amino acid sequence comprising the amino acid sequence of SEQ ID NO: 58, an amino acid sequence having at least 99.5%, 99%, 98.5%, 98%, 98.5% or 97% identity to the amino acid sequence of SEQ ID NO: 58, or an immunogenic fragment of the amino acid sequence of SEQ ID NO: 58, or the amino acid sequence having at least 99.5%, 99%, 98.5%, 98%, 98.5% or 97% identity to the amino acid sequence of SEQ ID NO: 58. In one embodiment, RNA encoding a vaccine antigen (i) comprises the nucleotide sequence of SEQ ID NO: 60; and/or (ii) encodes an amino acid sequence comprising the amino acid sequence of SEQ ID NO: 58.

In one embodiment, a vaccine antigen comprises the amino acid sequence of SEQ ID NO: 61, an amino acid sequence having at least 99.5%, 99%, 98.5%, 98%, 98.5% or 97% identity to the amino acid sequence of SEQ ID NO: 61, or an immunogenic fragment of the amino acid sequence of SEQ ID NO: 61, or the amino acid sequence having at least 99.5%, 99%, 98.5%, 98%, 98.5% or 97% identity to the amino acid sequence of SEQ ID NO: 61.

In one embodiment, a vaccine antigen comprises the amino acid sequence of SEQ ID NO: 61.

In one embodiment, RNA encoding a vaccine antigen (i) comprises the nucleotide sequence of SEQ ID NO: 62, a nucleotide sequence having at least 99.5%, 99%, 98.5%, 98%, 98.5% or 97% identity to the nucleotide sequence of SEQ ID NO: 62, or a fragment of the nucleotide sequence of SEQ ID NO: 62, or the nucleotide sequence having at least 99.5%, 99%, 98.5%, 98%, 98.5% or 97% identity to the nucleotide sequence of SEQ ID NO: 62; and/or (ii) encodes an amino acid sequence comprising the amino acid sequence of SEQ ID NO: 61, an amino acid sequence having at least 99.5%, 99%, 98.5%, 98%, 98.5% or 97% identity to the amino acid sequence of SEQ ID NO: 61, or an immunogenic fragment of the amino acid sequence of SEQ ID NO: 61, or the amino acid sequence having at least 99.5%, 99%, 98.5%, 98%, 98.5% or 97% identity to the amino acid sequence of SEQ ID NO: 61. In one embodiment, RNA encoding a vaccine antigen (i) comprises the nucleotide sequence of SEQ ID NO: 62; and/or (ii) encodes an amino acid sequence comprising the amino acid sequence of SEQ ID NO: 61.

In one embodiment, RNA encoding a vaccine antigen (i) comprises the nucleotide sequence of SEQ ID NO: 63, a nucleotide sequence having at least 99.5%, 99%, 98.5%, 98%, 98.5% or 97% identity to the nucleotide sequence of SEQ ID NO: 63, or a fragment of the nucleotide sequence of SEQ ID NO: 63, or the nucleotide sequence having at least 99.5%, 99%, 98.5%, 98%, 98.5% or 97% identity to the nucleotide sequence of SEQ ID NO: 63; and/or (ii) encodes an amino acid sequence comprising the amino acid sequence of SEQ ID NO: 61, an amino acid sequence having at least 99.5%, 99%, 98.5%, 98%, 98.5% or 97% identity to the amino acid sequence of SEQ ID NO: 61, or an immunogenic fragment of the amino acid sequence of SEQ ID NO: 61, or the amino acid sequence having at least 99.5%, 99%, 98.5%, 98%, 98.5% or 97% identity to the amino acid sequence of SEQ ID NO: 61. In one embodiment, RNA encoding a vaccine antigen (i) comprises the nucleotide sequence of SEQ ID NO: 63; and/or (ii) encodes an amino acid sequence comprising the amino acid sequence of SEQ ID NO: 61.

In one embodiment, a vaccine antigen comprises the amino acid sequence of SEQ ID NO: 52, an amino acid sequence having at least 99.5%, 99%, 98.5%, 98%, 98.5% or 97% identity to the amino acid sequence of SEQ ID NO: 52, or an immunogenic fragment of the amino acid sequence of SEQ ID NO: 52, or the amino acid sequence having at least 99.5%, 99%, 98.5%, 98%, 98.5% or 97% identity to the amino acid sequence of SEQ ID NO: 52.

In one embodiment, a vaccine antigen comprises the amino acid sequence of SEQ ID NO: 52.

In one embodiment, RNA encoding a vaccine antigen (i) comprises the nucleotide sequence of SEQ ID NO: 53, a nucleotide sequence having at least 99.5%, 99%, 98.5%, 98%, 98.5% or 97% identity to the nucleotide sequence of SEQ ID NO: 53, or a fragment of the nucleotide sequence of SEQ ID NO: 53, or the nucleotide sequence having at least 99.5%, 99%, 98.5%, 98%, 98.5% or 97% identity to the nucleotide sequence of SEQ ID NO: 53; and/or (ii) encodes an amino acid sequence comprising the amino acid sequence of SEQ ID NO: 52, an amino acid sequence having at least 99.5%, 99%, 98.5%, 98%, 98.5% or 97% identity to the amino acid sequence of SEQ ID NO: 52, or an immunogenic fragment of the amino acid sequence of SEQ ID NO: 52, or the amino acid sequence having at least 99.5%, 99%, 98.5%, 98%, 98.5% or 97% identity to the amino acid sequence of SEQ ID NO: 52. In one embodiment, RNA encoding a vaccine antigen (i) comprises the nucleotide sequence of SEQ ID NO: 53; and/or (ii) encodes an amino acid sequence comprising the amino acid sequence of SEQ ID NO: 52.

In one embodiment, RNA encoding a vaccine antigen (i) comprises the nucleotide sequence of SEQ ID NO: 54, a nucleotide sequence having at least 99.5%, 99%, 98.5%, 98%, 98.5% or 97% identity to the nucleotide sequence of SEQ ID NO: 54, or a fragment of the nucleotide sequence of SEQ ID NO: 54, or the nucleotide sequence having at least 99.5%, 99%, 98.5%, 98%, 98.5% or 97% identity to the nucleotide sequence of SEQ ID NO: 54; and/or (ii) encodes an amino acid sequence comprising the amino acid sequence of SEQ ID NO: 52, an amino acid sequence having at least 99.5%, 99%, 98.5%, 98%, 98.5% or 97% identity to the amino acid sequence of SEQ ID NO: 52, or an immunogenic fragment of the amino acid sequence of SEQ ID NO: 52, or the amino acid sequence having at least 99.5%, 99%, 98.5%, 98%, 98.5% or 97% identity to the amino acid sequence of SEQ ID NO: 52. In one embodiment, RNA encoding a vaccine antigen (i) comprises the nucleotide sequence of SEQ ID NO: 54; and/or (ii) encodes an amino acid sequence comprising the amino acid sequence of SEQ ID NO: 52.

In one embodiment, the vaccine antigens described above comprise a contiguous sequence of SARS-CoV-2 coronavirus spike(S) protein that consists of or essentially consists of the above described amino acid sequences derived from SARS-CoV-2 S protein or immunogenic fragments thereof (antigenic peptides or proteins) comprised by the vaccine antigens described above. In one embodiment, the vaccine antigens described above comprise a contiguous sequence of SARS-CoV-2 coronavirus spike(S) protein of no more than 220 amino acids, 215 amino acids, 210 amino acids, or 205 amino acids.

In one embodiment, RNA encoding a vaccine antigen is nucleoside modified messenger RNA (modRNA) described herein as BNT162b1 (RBP020.3), BNT162b2 (RBP020.1 or RBP020.2), or BNT162b3 (e.g., BNT162b3c). In one embodiment, RNA encoding a vaccine antigen is nucleoside modified messenger RNA (modRNA) described herein as RBP020.2. In one embodiment, RNA encoding a vaccine antigen is nucleoside modified messenger RNA (modRNA) described herein as BNT162b3 (e.g., BNT162b3c).

In one embodiment, RNA encoding a vaccine antigen is nucleoside modified messenger RNA (modRNA) and (i) comprises the nucleotide sequence of SEQ ID NO: 21, a nucleotide sequence having at least 99%, 98%, 97%, 96%, 95%, 90%, 85%, or 80% identity to the nucleotide sequence of SEQ ID NO: 21, and/or (ii) encodes an amino acid sequence comprising the amino acid sequence of SEQ ID NO: 5, or an amino acid sequence having at least 99%, 98%, 97%, 96%, 95%, 90%, 85%, or 80% identity to the amino acid sequence of SEQ ID NO: 5. In one embodiment, RNA encoding a vaccine antigen is nucleoside modified messenger RNA (modRNA) and (i) comprises the nucleotide sequence of SEQ ID NO: 21; and/or (ii) encodes an amino acid sequence comprising the amino acid sequence of SEQ ID NO: 5.

In one embodiment, RNA encoding a vaccine antigen is nucleoside modified messenger RNA (modRNA) and (i) comprises the nucleotide sequence of SEQ ID NO: 19, or 20, a nucleotide sequence having at least 99%, 98%, 97%, 96%, 95%, 90%, 85%, or 80% identity to the nucleotide sequence of SEQ ID NO: 19, or 20, and/or (ii) encodes an amino acid sequence comprising the amino acid sequence of SEQ ID NO: 7, or an amino acid sequence having at least 99%, 98%, 97%, 96%, 95%, 90%, 85%, or 80% identity to the amino acid sequence of SEQ ID NO: 7. In one embodiment, RNA encoding a vaccine antigen is nucleoside modified messenger RNA (modRNA) and (i) comprises the nucleotide sequence of SEQ ID NO: 19, or 20; and/or (ii) encodes an amino acid sequence comprising the amino acid sequence of SEQ ID NO: 7.

In one embodiment, RNA encoding a vaccine antigen is nucleoside modified messenger RNA (modRNA) and (i) comprises the nucleotide sequence of SEQ ID NO: 20, a nucleotide sequence having at least 99%, 98%, 97%, 96%, 95%, 90%, 85%, or 80% identity to the nucleotide sequence of SEQ ID NO: 20, and/or (ii) encodes an amino acid sequence comprising the amino acid sequence of SEQ ID NO: 7, or an amino acid sequence having at least 99%, 98%, 97%, 96%, 95%, 90%, 85%, or 80% identity to the amino acid sequence of SEQ ID NO: 7. In one embodiment, RNA encoding a vaccine antigen is nucleoside modified messenger RNA (modRNA) and (i) comprises the nucleotide sequence of SEQ ID NO: 20; and/or (ii) encodes an amino acid sequence comprising the amino acid sequence of SEQ ID NO: 7.

In one embodiment, RNA encoding a vaccine antigen is nucleoside modified messenger RNA (modRNA) and (i) comprises the nucleotide sequence of SEQ ID NO: 30, a nucleotide sequence having at least 99%, 98%, 97%, 96%, 95%, 90%, 85%, or 80% identity to the nucleotide sequence of SEQ ID NO: 30, and/or (ii) encodes an amino acid sequence comprising the amino acid sequence of SEQ ID NO: 29, or an amino acid sequence having at least 99%, 98%, 97%, 96%, 95%, 90%, 85%, or 80% identity to the amino acid sequence of SEQ ID NO: 29. In one embodiment, RNA encoding a vaccine antigen is nucleoside modified messenger RNA (modRNA) and (i) comprises the nucleotide sequence of SEQ ID NO: 30; and/or (ii) encodes an amino acid sequence comprising the amino acid sequence of SEQ ID NO: 29.

In one embodiment, RNA encoding a vaccine antigen is nucleoside modified messenger RNA (modRNA) and (i) comprises the nucleotide sequence of SEQ ID NO: 50, a nucleotide sequence having at least 99.5%, 99%, 98.5%, 98%, 98.5% or 97% identity to the nucleotide sequence of SEQ ID NO: 50, and/or (ii) encodes an amino acid sequence comprising the amino acid sequence of SEQ ID NO: 49, or an amino acid sequence having at least 99.5%, 99%, 98.5%, 98%, 98.5% or 97% identity to the amino acid sequence of SEQ ID NO: 49. In one embodiment, RNA encoding a vaccine antigen is nucleoside modified messenger RNA (modRNA) and (i) comprises the nucleotide sequence of SEQ ID NO: 50; and/or (ii) encodes an amino acid sequence comprising the amino acid sequence of SEQ ID NO: 49.

In one embodiment, RNA encoding a vaccine antigen is nucleoside modified messenger RNA (modRNA) and (i) comprises the nucleotide sequence of SEQ ID NO: 51, a nucleotide sequence having at least 99.5%, 99%, 98.5%, 98%, 98.5% or 97% identity to the nucleotide sequence of SEQ ID NO: 51, and/or (ii) encodes an amino acid sequence comprising the amino acid sequence of SEQ ID NO: 49, or an amino acid sequence having at least 99.5%, 99%, 98.5%, 98%, 98.5% or 97% identity to the amino acid sequence of SEQ ID NO: 49. In one embodiment, RNA encoding a vaccine antigen is nucleoside modified messenger RNA (modRNA) and (i) comprises the nucleotide sequence of SEQ ID NO: 51; and/or (ii) encodes an amino acid sequence comprising the amino acid sequence of SEQ ID NO: 49.

In one embodiment, RNA encoding a vaccine antigen is nucleoside modified messenger RNA (modRNA) and (i) comprises the nucleotide sequence of SEQ ID NO: 57, a nucleotide sequence having at least 99.5%, 99%, 98.5%, 98%, 98.5% or 97% identity to the nucleotide sequence of SEQ ID NO: 57, and/or (ii) encodes an amino acid sequence comprising the amino acid sequence of SEQ ID NO: 55, or an amino acid sequence having at least 99.5%, 99%, 98.5%, 98%, 98.5% or 97% identity to the amino acid sequence of SEQ ID NO: 55. In one embodiment, RNA encoding a vaccine antigen is nucleoside modified messenger RNA (modRNA) and (i) comprises the nucleotide sequence of SEQ ID NO: 57; and/or (ii) encodes an amino acid sequence comprising the amino acid sequence of SEQ ID NO: 55.

In one embodiment, RNA encoding a vaccine antigen is nucleoside modified messenger RNA (modRNA) and (i) comprises the nucleotide sequence of SEQ ID NO: 60, a nucleotide sequence having at least 99.5%, 99%, 98.5%, 98%, 98.5% or 97% identity to the nucleotide sequence of SEQ ID NO: 60, and/or (ii) encodes an amino acid sequence comprising the amino acid sequence of SEQ ID NO: 58, or an amino acid sequence having at least 99.5%, 99%, 98.5%, 98%, 98.5% or 97% identity to the amino acid sequence of SEQ ID NO: 58. In one embodiment, RNA encoding a vaccine antigen is nucleoside modified messenger RNA (modRNA) and (i) comprises the nucleotide sequence of SEQ ID NO: 60; and/or (ii) encodes an amino acid sequence comprising the amino acid sequence of SEQ ID NO: 58.

In one embodiment, RNA encoding a vaccine antigen is nucleoside modified messenger RNA (modRNA) and (i) comprises the nucleotide sequence of SEQ ID NO: 63, a nucleotide sequence having at least 99.5%, 99%, 98.5%, 98%, 98.5% or 97% identity to the nucleotide sequence of SEQ ID NO: 63, and/or (ii) encodes an amino acid sequence comprising the amino acid sequence of SEQ ID NO: 61, or an amino acid sequence having at least 99.5%, 99%, 98.5%, 98%, 98.5% or 97% identity to the amino acid sequence of SEQ ID NO: 61. In one embodiment, RNA encoding a vaccine antigen is nucleoside modified messenger RNA (modRNA) and (i) comprises the nucleotide sequence of SEQ ID NO: 63; and/or (ii) encodes an amino acid sequence comprising the amino acid sequence of SEQ ID NO: 61.

In one embodiment, RNA encoding a vaccine antigen is nucleoside modified messenger RNA (modRNA) and (i) comprises the nucleotide sequence of SEQ ID NO: 53, a nucleotide sequence having at least 99.5%, 99%, 98.5%, 98%, 98.5% or 97% identity to the nucleotide sequence of SEQ ID NO: 53, and/or (ii) encodes an amino acid sequence comprising the amino acid sequence of SEQ ID NO: 52, or an amino acid sequence having at least 99.5%, 99%, 98.5%, 98%, 98.5% or 97% identity to the amino acid sequence of SEQ ID NO: 52. In one embodiment, RNA encoding a vaccine antigen is nucleoside modified messenger RNA (modRNA) and (i) comprises the nucleotide sequence of SEQ ID NO: 53; and/or (ii) encodes an amino acid sequence comprising the amino acid sequence of SEQ ID NO: 52.

In one embodiment, RNA encoding a vaccine antigen is nucleoside modified messenger RNA (modRNA) and (i) comprises the nucleotide sequence of SEQ ID NO: 54, a nucleotide sequence having at least 99.5%, 99%, 98.5%, 98%, 98.5% or 97% identity to the nucleotide sequence of SEQ ID NO: 54, and/or (ii) encodes an amino acid sequence comprising the amino acid sequence of SEQ ID NO: 52, or an amino acid sequence having at least 99.5%, 99%, 98.5%, 98%, 98.5% or 97% identity to the amino acid sequence of SEQ ID NO: 52. In one embodiment, RNA encoding a vaccine antigen is nucleoside modified messenger RNA (modRNA) and (i) comprises the nucleotide sequence of SEQ ID NO: 54; and/or (ii) encodes an amino acid sequence comprising the amino acid sequence of SEQ ID NO: 52.

As used herein, the term “vaccine” refers to a composition that induces an immune response upon inoculation into a subject. In some embodiments, the induced immune response provides protective immunity.

In one embodiment, RNA encoding an antigen molecule is expressed in cells of the subject to provide the antigen molecule. In one embodiment, expression of the antigen molecule is at the cell surface or into the extracellular space. In one embodiment, the antigen molecule is presented in the context of MHC. In one embodiment, an RNA encoding an antigen molecule is transiently expressed in cells of the subject. In one embodiment, after administration of an RNA encoding the antigen molecule, in particular after intramuscular administration of an RNA encoding an antigen molecule, expression of the RNA encoding the antigen molecule in muscle occurs. In one embodiment, after administration of an RNA encoding the antigen molecule, expression of the RNA encoding the antigen molecule in spleen occurs. In one embodiment, after administration of an RNA encoding the antigen molecule, expression of the RNA encoding the antigen molecule in antigen presenting cells, preferably professional antigen presenting cells occurs. In one embodiment, the antigen presenting cells are selected from the group consisting of dendritic cells, macrophages and B cells. In one embodiment, after administration of an RNA encoding the antigen molecule, no or essentially no expression of the RNA encoding the antigen molecule in lung and/or liver occurs. In one embodiment, after administration of an RNA encoding the antigen molecule, expression of the RNA encoding the antigen molecule in spleen is at least 5-fold the amount of expression in lung.

In some embodiments, the methods and agents, e.g., mRNA compositions, described herein following administration, in particular following intramuscular administration, to a subject result in delivery of an RNA encoding a vaccine antigen to lymph nodes and/or spleen. In some embodiments, RNA encoding a vaccine antigen is detectable in lymph nodes and/or spleen 6 hours or later following administration and preferably up to 6 days or longer.

In some embodiments, the methods and agents, e.g., mRNA compositions, described herein following administration, in particular following intramuscular administration, to a subject result in delivery of an RNA encoding a vaccine antigen to B cell follicles, subcapsular sinus, and/or T cell zone, in particular B cell follicles and/or subcapsular sinus of lymph nodes.

In some embodiments, the methods and agents, e.g., mRNA compositions, described herein following administration, in particular following intramuscular administration, to a subject result in delivery of RNA encoding a vaccine antigen to B cells (CD19+), subcapsular sinus macrophages (CD169+) and/or dendritic cells (CD11c+) in the T cell zone and intermediary sinus of lymph nodes, in particular to B cells (CD19+) and/or subcapsular sinus macrophages (CD169+) of lymph nodes.

In some embodiments, the methods and agents, e.g., mRNA compositions, described herein following administration, in particular following intramuscular administration, to a subject result in delivery of RNA encoding a vaccine antigen to white pulp of spleen.

In some embodiments, the methods and agents, e.g., mRNA compositions, described herein following administration, in particular following intramuscular administration, to a subject result in delivery of RNA encoding a vaccine antigen to B cells, DCs (CD11c+), in particular those surrounding the B cells, and/or macrophages of spleen, in particular to B cells and/or DCs (CD11c+).

In one embodiment, the vaccine antigen is expressed in lymph node and/or spleen, in particular in the cells of lymph node and/or spleen described above.

The peptide and protein antigens suitable for use according to the present disclosure typically include a peptide or protein comprising an epitope of SARS-CoV-2 S protein or a functional variant thereof for inducing an immune response. The peptide or protein or epitope may be derived from a target antigen, i.e. the antigen against which an immune response is to be elicited. For example, the peptide or protein antigen or the epitope contained within the peptide or protein antigen may be a target antigen or a fragment or variant of a target antigen. The target antigen may be a coronavirus S protein, in particular SARS-CoV-2 S protein.

The antigen molecule or a procession product thereof, e.g., a fragment thereof, may bind to an antigen receptor such as a BCR or TCR carried by immune effector cells, or to antibodies.

A peptide and protein antigen which is provided to a subject according to the present disclosure by administering RNA encoding the peptide and protein antigen, i.e., a vaccine antigen, preferably results in the induction of an immune response, e.g., a humoral and/or cellular immune response in the subject being provided the peptide or protein antigen. Said immune response is preferably directed against a target antigen, in particular a coronavirus S protein, in particular SARS-CoV-2 S protein and/or an influenza HA protein. Thus, a vaccine antigen may comprise the target antigen, a variant thereof, or a fragment thereof. In one embodiment, such fragment or variant is immunologically equivalent to the target antigen. In the context of the present disclosure, the term “fragment of an antigen” or “variant of an antigen” means an agent which results in the induction of an immune response which immune response targets the antigen, i.e. a target antigen. Thus, the vaccine antigen may correspond to or may comprise the target antigen, may correspond to or may comprise a fragment of the target antigen or may correspond to or may comprise an antigen which is homologous to the target antigen or a fragment thereof. Thus, according to the present disclosure, a vaccine antigen may comprise an immunogenic fragment of a target antigen or an amino acid sequence being homologous to an immunogenic fragment of a target antigen. An “immunogenic fragment of an antigen” according to the present disclosure preferably relates to a fragment of an antigen which is capable of inducing an immune response against the target antigen. The vaccine antigen may be a recombinant antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization.

For example, an amino acid sequence is immunologically equivalent to a reference amino acid sequence if said amino acid sequence when exposed to the immune system of a subject induces an immune reaction having a specificity of reacting with the reference amino acid sequence.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which immune effector cells are stimulated by an antigen, proliferate, and the specific immune effector cell recognizing said antigen is amplified. Preferably, clonal expansion leads to differentiation of the immune effector cells.

The term “antigen” relates to an agent comprising an epitope against which an immune response can be generated.

The term “antigen” includes, in particular, proteins and peptides. In one embodiment, an antigen is presented by cells of the immune system such as antigen presenting cells like dendritic cells or macrophages. An antigen or a procession product thereof such as a T-cell epitope is in one embodiment bound by a T- or B-cell receptor, or by an immunoglobulin molecule such as an antibody. Accordingly, an antigen or a procession product thereof may react specifically with antibodies or T lymphocytes (T cells). In one embodiment, an antigen is a viral antigen, such as a coronavirus S protein, e.g., SARS-CoV-2 S protein, and an epitope is derived from such antigen.

The term “viral antigen” refers to any viral component having antigenic properties, i.e. being able to provoke an immune response in an individual. The viral antigen may be coronavirus S protein, e.g., SARS-CoV-2 S protein. The viral antigen may be an influenza protein, e.g., an HA protein.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by e.g. antigen-specific antibodies added to the cells.

The term “extracellular portion” or “exodomain” in the context of the present disclosure refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and protein antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to the peptide or protein.

In one embodiment, a vaccine antigen is recognized by an immune effector cell. Preferably, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen is preferably presented or present on the surface of a cell, preferably an antigen presenting cell. In one embodiment, an antigen is presented by a diseased cell such as a virus-infected cell. In one embodiment, an antigen receptor is a TCR which binds to an epitope of an antigen presented in the context of MHC. In one embodiment, binding of a TCR when expressed by T cells and/or present on T cells to an antigen presented by cells such as antigen presenting cells results in stimulation, priming and/or expansion of said T cells. In one embodiment, binding of a TCR when expressed by T cells and/or present on T cells to an antigen presented on diseased cells results in cytolysis and/or apoptosis of the diseased cells, wherein said T cells preferably release cytotoxic factors, e.g. perforins and granzymes.

In one embodiment, an antigen receptor is an antibody or B cell receptor which binds to an epitope in an antigen.

In one embodiment, an antibody or B cell receptor binds to native epitopes of an antigen.

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the present disclosure, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment of all aspects of the present disclosure, RNA encoding the vaccine antigen is expressed in cells such as antigen presenting cells of the subject treated to provide the vaccine antigen.

The nucleic acids described herein may be recombinant and/or isolated molecules.

In the present disclosure, the term “RNA” relates to a nucleic acid molecule which includes ribonucleotide residues. In preferred embodiments, RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, an RNA is a messenger RNA (mRNA), which relates to an RNA transcript which encodes a peptide or protein. As established in the art, mRNA generally contains a 5′ untranslated region (5′-UTR), a peptide coding region and a 3′ untranslated region (3′-UTR). In some embodiments, RNA is produced by in vitro transcription or chemical synthesis. In one embodiment, mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In certain embodiments of the present disclosure, an RNA is “replicon RNA” or simply a “replicon”, in particular “self-replicating RNA” or “self-amplifying RNA”. In one particularly preferred embodiment, the replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In one embodiment, RNA described herein may have modified nucleosides. In some embodiments, RNA comprises a modified nucleoside in place of at least one (e.g., every) uridine.

The term “uracil,” as used herein, describes one of the nucleobases that can occur in the nucleic acid of RNA. The structure of uracil is:

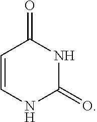

The term “uridine,” as used herein, describes one of the nucleosides that can occur in RNA. The structure of uridine is:

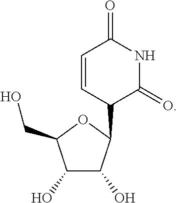 [SMILES: O=C1C=CC([C@@H]2O[C@H](CO)[C@@H](O)[C@H]2O)C(=O)N1]

UTP (uridine 5′-triphosphate) has the following structure:

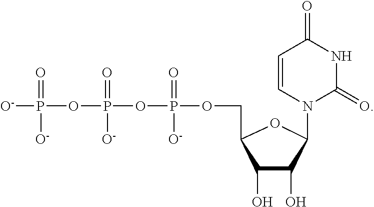

Pseudo-UTP (pseudouridine 5′-triphosphate) has the following structure:

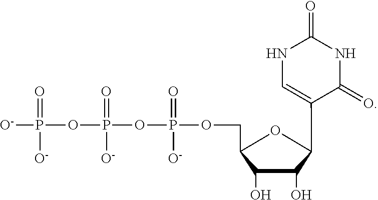

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

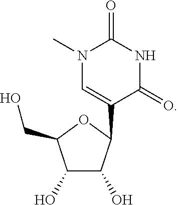 [SMILES: Cn1cc([C@@H]2O[C@H](CO)[C@@H](O)[C@H]2O)c(=O)[nH]c1=O]

N1-methyl-pseudo-UTP has the following structure:

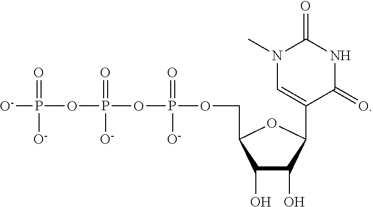

Another exemplary modified nucleoside is 5-methyl-uridine (m5U), which has the structure:

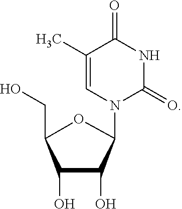

In some embodiments, one or more uridine in an RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in an RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (Tm⁵U), 1-taurinomethyl-pseudouridine, 5-taurinomethyl-2-thio-uridine (Tm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, or any other modified uridine known in the art.

In one embodiment, an RNA comprises other modified nucleosides or comprises further modified nucleosides, e.g., modified cytidine. For example, in one embodiment, in an RNA, 5-methylcytidine is substituted partially or completely, preferably completely, for cytidine. In one embodiment, an RNA comprises 5-methylcytidine and one or more selected from pseudouridine (ψ), N1-methyl-pseudouridine (m1ψ), and 5-methyl-uridine (m5U). In one embodiment, an RNA comprises 5-methylcytidine and N1-methyl-pseudouridine (m1ψ). In some embodiments, an RNA comprises 5-methylcytidine in place of each cytidine and N1-methyl-pseudouridine (m1ψ) in place of each uridine.

### 5′ Caps

In some embodiments, an RNA described herein comprises a 5′ cap. Natural eukaryotic mRNA comprises a 7-methylguanosine cap linked to the mRNA via a 5′ to 5 ‘-triphosphate bridge resulting in cap0 structure (m7GpppN). In most eukaryotic mRNA and some viral mRNA, further modifications can occur at the 2’-hydroxy-group (2′-OH) (e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550 reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate IFN response. Daffis, et al. (2010) Nature, 468:452-456; and Züst et al. (2011) Nature Immunology, 12:137-143.

RNA capping is well researched and is described, e.g., in Decroly E et al. (2012) Nature Reviews 10:51-65; and in Ramanathan A. et al., (2016) Nucleic Acids Res; 44(16): 7511-7526, the entire contents of each of which is hereby incorporated by reference. For example, in some embodiments, a 5′-cap structure which may be suitable in the context of the present invention is a cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In one embodiment, RNA of the present disclosure does not have uncapped 5′-triphosphates. In one embodiment, RNA may be modified by a 5′-cap analog.

The term “5′-cap” refers to a structure found on the 5′-end of an RNA (e.g., mRNA) molecule and generally includes a guanosine nucleotide connected to an RNA (e.g., mRNA) via a 5′- to 5′-triphosphate linkage (also referred to as Gppp or G (5′) ppp (5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′O methylation at a ribose (3′OMeG). In one embodiment, this guanosine is methylated at the 7-position. In some embodiments, providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes. In some embodiments, co-transcriptional capping with a cap disclosed improves the capping efficiency of an RNA compared to co-transcriptional capping with an appropriate reference comparator. In some embodiments, improving capping efficiency can increase translation efficiency and/or translation rate of an RNA, and/or increase expression of an encoded polypeptide. In some embodiments, alterations to polynucleotides generates a non-hydrolyzable cap structure which can, for example, prevent decapping and increase RNA half-life.

In some embodiments, T7 RNA polymerase prefers G as the initial site. Accordingly, in some such embodiments, the present disclosure provides caps (e.g., trinucleotide and tetranucleotide caps described herein) wherein the 3′end of the trinucleotide (e.g., N₂) or tetranucleotide cap (e.g., N₃) is G.

In some embodiments, it will be appreciated that all compounds or structures (e.g., 5′ caps) provided herein encompass the free base or a salt form (e.g., an Na⁺ salt) comprising a suitable counterion (e.g., Na⁺). Compounds or structures (e.g., 5′ caps) depicted as a salt also encompass the free base and include suitable counterions (e.g., Na⁺).

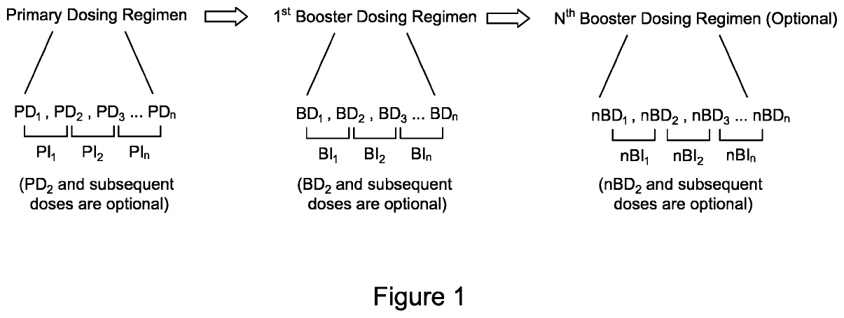

In some embodiments, a utilized 5′ cap is a cap0, a cap1, or cap2 structure. See, e.g., FIG. 1 of Ramanathan A et al., and FIG. 1 of Decroly E et al., each of which is incorporated herein by reference in its entirety. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2 structure.

In some embodiments, an RNA described herein comprises a cap0 structure. In some embodiments, a cap0 structure comprises a guanosine nucleoside methylated at the 7-position of guanine ((m⁷)G). In some embodiments, such a cap0 structure is connected to an RNA via a 5′- to 5′-triphosphate linkage and is also referred to herein as (m⁷)Gppp. In some embodiments, a cap0 structure comprises a guanosine nucleoside methylated at the 2′-position of the ribose of guanosine In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m₂^{7,2′-O})G). In some embodiments, a guanosine nucleoside included in a 5′ cap comprises methylation at the 7-position of guanine and at the 2′-position of the ribose ((m₂^{7,3′-O})G).

In some embodiments, an RNA described herein comprises a cap1 structure. In some embodiments, a cap1 structure comprises a guanosine nucleoside methylated at the 7-position of guanine ((m⁷)G) and optionally methylated at the 2′ or 3′ position of the ribose, and a 2′O methylated first nucleotide in an RNA ((m^{2′-O})N₁). In some embodiments, a cap1 structure comprises a guanosine nucleoside methylated at the 7-position of guanine ((m⁷)G) and the 3′ position of the ribose, and a 2′O methylated first nucleotide in an RNA ((m^{2′-O})N₁). In some embodiments, a cap1 structure is connected to an RNA via a 5′- to 5′-triphosphate linkage and is also referred to herein as, e.g., ((m⁷)Gppp(^{2′-O})N₁) or (m₂^{7,3′-O})Gppp(^{2′-O})N₁), wherein N₁ is as defined and described herein. In some embodiments, a cap1 structure comprises a second nucleotide, N2, which is at position 2 and is chosen from A, G, C, or U, e.g., (m⁷)Gpp(^{2′-O})N₁pN₂ or (m₂^{7,3′-O})Gppp(^{2′-O})N₁pN₂, wherein each of N₁ and N₂ is as defined and described herein.

In some embodiments, an RNA described herein comprises a cap2 structure. In some embodiments, a cap2 structure comprises a guanosine nucleoside methylated at the 7-position of guanine ((m⁷)G) and optionally methylated at the 2′ or 3′ position of the ribose, and a 2′O methylated first and second nucleotides in an RNA ((m^{2′-O})N₁p(m^{2′-O})N₂). In some embodiments, a cap2 structure comprises a guanosine nucleoside methylated at the 7-position of guanine ((m⁷)G) and the 3′ position of the ribose, and a 2′O methylated first and second nucleotide in an RNA. In some embodiments, a cap2 structure is connected to an RNA via a 5′- to 5′-triphosphate linkage and is also referred to herein as, e.g., ((m⁷)Gppp(^{2′-O})N₁p(^{2′-O})N₂) or (m₂^{7,3′-O})Gppp(^{2′-O})N₁p(^{2′-O})N₂), wherein each of N₁ and N₂ is as defined and described herein.

In some embodiments, a 5′ cap is a dinucleotide cap structure. In some embodiments, a 5′ cap is a dinucleotide cap structure comprising N₁, wherein N₁ is as defined and described herein. In some embodiments, a 5′ cap is a dinucleotide cap G*N₁, wherein N₁ is as defined above and herein, and:
G* comprises a structure of formula (I):

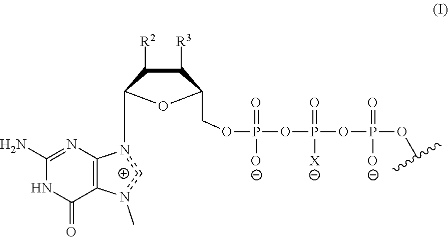

or a salt thereof,
wherein
each R² and R³ is —OH or —OCH₃; and
X is O or S.

In some embodiments, R² is —OH. In some embodiments, R² is —OCH₃. In some embodiments, R³ is —OH. In some embodiments, R³ is —OCH₃. In some embodiments, R² is —OH and R³ is —OH. In some embodiments, R² is —OH and R³ is —CH₃. In some embodiments, R² is —CH₃ and R³ is —OH. In some embodiments, R² is —CH₃ and R³ is —CH₃.

In some embodiments, a guanosine nucleoside included in a 5′ cap comprises methylation at the 7-position of guanine and a 3′ O methylation at a ribose (m7(3′OMeG)). It will be understood that the notation used in the above paragraph, e.g., “(m₂^{7,3′-O})G” or “m7(3′OMeG)”, applies to other structures described herein.

In some embodiments, a 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is as defined and described herein. In some embodiments, a 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is G. In some embodiments, a 5′ cap is a dinucleotide cap0 structure (e.g., (m⁷)GpppN₁, (m₂^{7,2′-O})GpppN₁, (m₂^{7,3′-O})GpppN₁, (m⁷)GppSpN₁, (m₂^{7,2′-O})GppSpN₁, or (m₂^{7,3′-O})GppSpN₁), wherein N₁ is A, U, or C. In some embodiments, a 5′ cap is a dinucleotide cap1 structure (e.g., (m⁷)Gppp(m^{2′-O})N₁, (m₂^{7,2′-O})Gppp(m^{2′-O}N₁, (m₂^{7,3′-O})Gppp(m^{2′-O}N₁, (m⁷)GppSp(m^{2′-O})N₁, (m₂^{7,2′-O})GppSp(m^{2′-O})N₁, or (m₂^{7,3′-O})GppSp(m^{2′-O})N₁), wherein N₁ is as defined and described herein. In some embodiments, a 5′ cap is selected from the group consisting of (m⁷)GpppG (“Ecap0”), (m⁷)Gppp(m^{2′-O})G (“Ecap1”), (m₂^{7,3′-O})GpppG (“ARCA” or “D1”), and (m₂^{7,2′-O})GppSpG (“beta-S-ARCA”). In some embodiments, a 5′ cap is (m⁷)GpppG (“Ecap0”), having a structure:

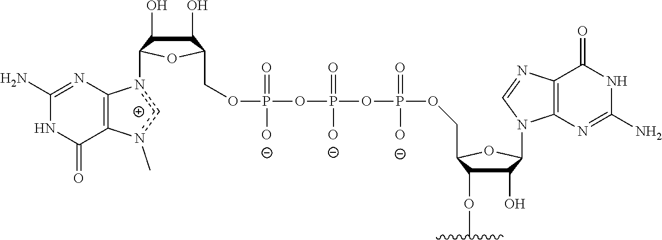

or a salt thereof.

In some embodiments, a 5′ cap is (m⁷)Gppp(m^{2′-O})G (“Ecap1”), having a structure:

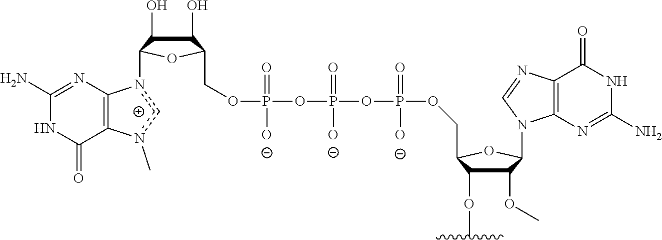

or a salt thereof.

In some embodiments, a 5′ cap is (m₂^{7,3′-O})GpppG (“ARCA” or “D1”), having a structure:

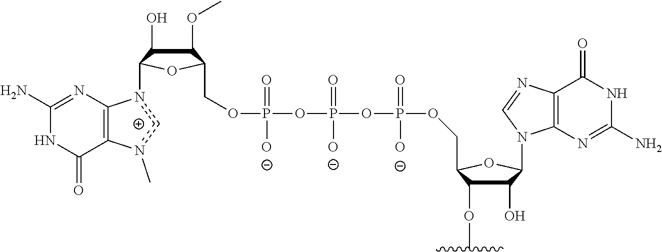

or a salt thereof.

In some embodiments, a 5′ cap is (m₂^{7,2′-O})GppSpG (“beta-S-ARCA”), having a structure:

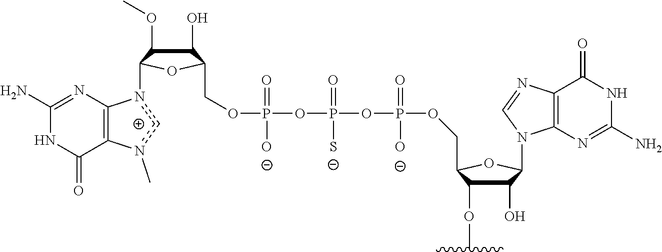

or a salt thereof.

In some embodiments, a 5′ cap is a trinucleotide cap structure. In some embodiments, a 5′ cap is a trinucleotide cap structure comprising N₁pN₂, wherein N₁ and N₂ are as defined and described herein. In some embodiments, a 5′ cap is a trinucleotide cap G*N₁pN₂, wherein N₁ and N₂ are as defined above and herein, and:
G* comprises a structure of formula (I):

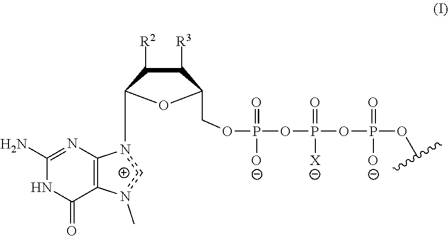

or a salt thereof, wherein R2, R³, and X are as defined and described herein.

In some embodiments, a 5′ cap is a trinucleotide cap0 structure (e.g. (m⁷)GppN₁pN₂, (m₂^{7,2′-O})GppN₁pN₂, or (m₂^{7,3′-O})GpppN₁pN₂), wherein N₁ and N₂ are as defined and described herein). In some embodiments, a 5′ cap is a trinucleotide cap1 structure (e.g., (m⁷)Gppp(m^{2′-O})N₁pN₂, (m₂^{7,2′-O})Gppp(m^{2′-O})N₁pN₂, (m₂^{7,3′-O})Gppp(m^{2′-O})N₁pN₂), wherein N₁ and N₂ are as defined and described herein. In some embodiments, a 5′ cap is a trinucleotide cap2 structure (e.g., (m⁷)Gppp(m^{2′-O})N₁p(m^{2′-O})N₂, (m₂^{7,2′-O})Gppp(m^{2′-O})N₁p(m^{2′-O})N₂, (m₂^{7,3′-O})Gppp(m^{2′-O})N₁p(m^{2′-O})N₂), wherein N₁ and N₂ are as defined and described herein. In some embodiments, a 5′ cap is selected from the group consisting of (m₂^{7,3′-O})Gppp(m^{2′-O})ApG (“CleanCap AG 3′ OMe”, “CC413”), (m₂^{7,3′-O})Gppp(m^{2′-O})GpG (“CleanCap GG”), (m⁷)Gppp(m^{2′-O})ApG, (m⁷)Gppp(m^{2′-O})GpG, (m₂^{7,3′-O})Gppp(m₂^{6,2′-O})ApG, and (m⁷)Gppp(m^{2′-O})ApU. In some embodiments, a 5′ cap is selected from the group consisting of (m₂^{7,3′-O})Gppp(m^{2′-O})ApG (“CleanCap AG”, “CC413”), (m₂^{7,3′-O})Gppp(m^{2′-O})GpG (“CleanCap GG”), (m⁷)Gppp(m^{2′-O})ApG, and (m₂^{7,3′-O})Gppp(m₂^{6,2′-O})ApG, (m⁷)Gppp(m^{2′-O})ApU, and (m₂^{7,3′-O})Gppp(m^{2′-O})CpG.

In some embodiments, a 5′ cap is (m₂^{7,3′-O})Gppp(m^{2′-O})ApG (“CleanCap AG 3′ OMe”, “CC413”), having a structure:

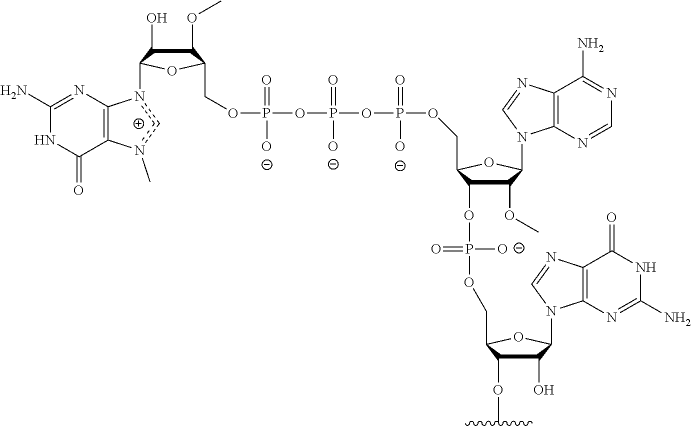

or a salt thereof.

In some embodiments, a 5′ cap is (m₂^{7,3′-O})Gpp (m^{2′-O})GpG (“CleanCap GG”), having a structure:

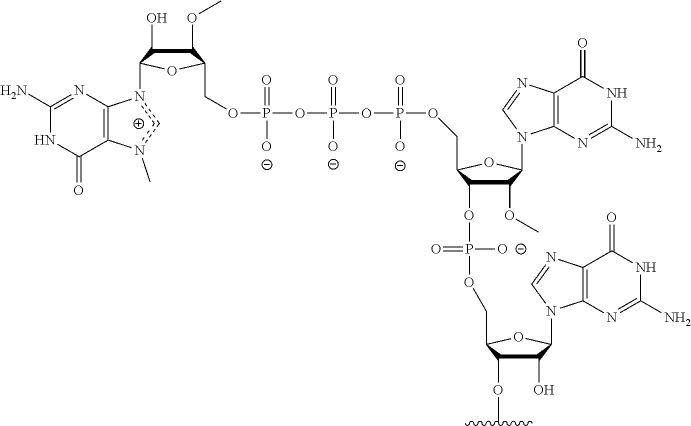

or a salt thereof.

In some embodiments, a 5′ cap is (m⁷)Gppp(m^{2′-O})ApG, having a structure:

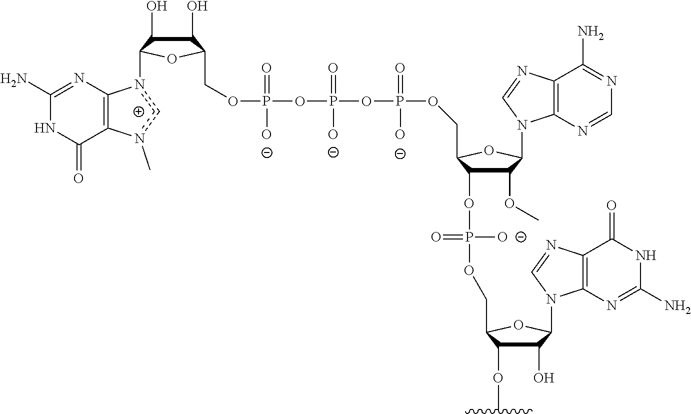

or a salt thereof.

In some embodiments, a 5′ cap is (m⁷)Gppp(m^{2′-O})GpG, having a structure:

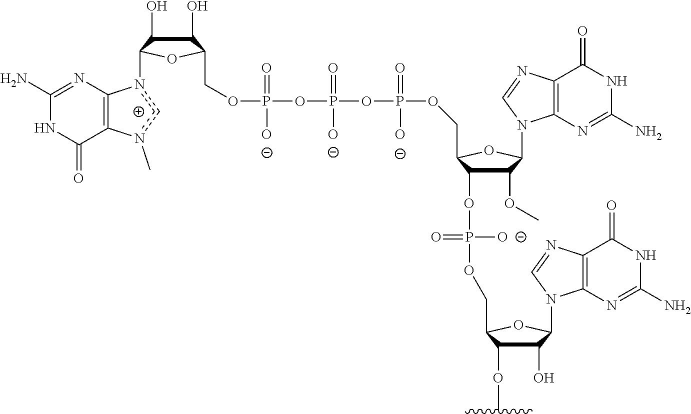

or a salt thereof.

In some embodiments, a 5′ cap is (m₂^{7,3′-O})Gppp(m₂^{6,2′-O})ApG, having a structure:

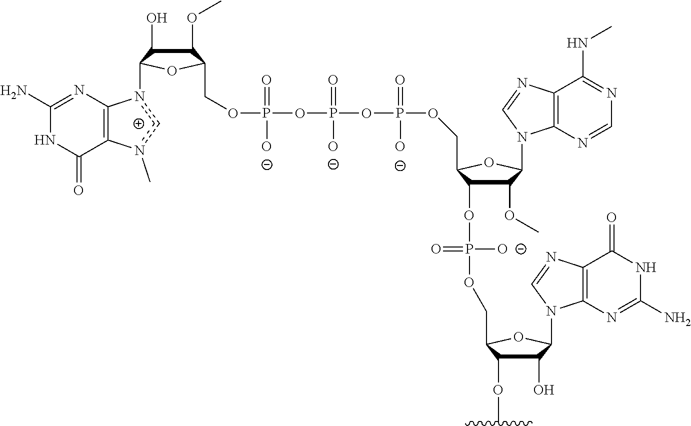

or a salt thereof.

In some embodiments, a 5′ cap is (m⁷)Gppp(m^{2′-O})ApU, having a structure:

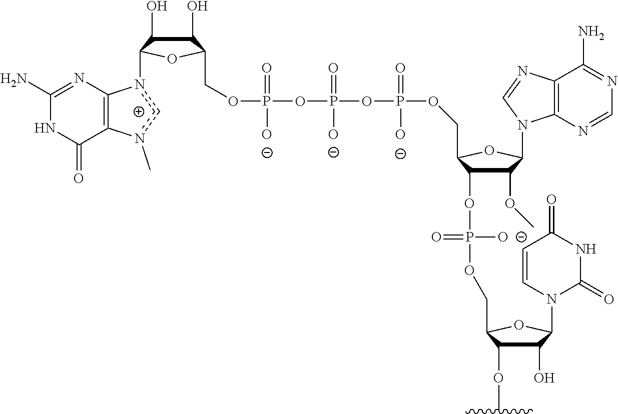

or a salt thereof.

In some embodiments, a 5′ cap is (m₂^{7,3′-O})Gppp(m^{2′-O})CpG, having a structure:

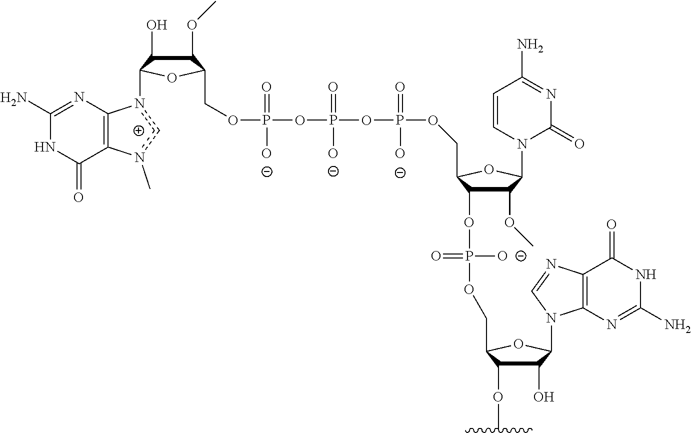

or a salt thereof.

In some embodiments, a 5′ cap is a tetranucleotide cap structure. In some embodiments, a 5′ cap is a tetranucleotide cap structure comprising N₁pN₂pN₃, wherein N₁, N₂, and N₃ are as defined and described herein.

In some embodiments, the 5′ cap is a tetranucleotide cap G*N₁pN₂pN₃, wherein N₁, N₂, and N₃ are as defined above and herein, and:
G* comprises a structure of formula (I):

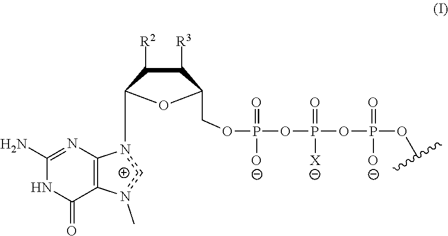

or a salt thereof, wherein R², R³, and X are as defined and described herein.

In some embodiments, a 5′ cap is a tetranucleotide cap0 structure (e.g. (m⁷)GpppN₁pN₂pN₃, (m₂^{7,2′-O})GpppN₁pN₂pN₃, or (m₂^{7,3′-O})GpppN₁N₂pN₃), wherein N₁, N₂, and N₃ are as defined and described herein). In some embodiments, a 5′ cap is a tetranucleotide Cap1 structure (e.g., (m⁷)Gppp(m^{2′-O})N₁pN₂pN₃, (m₂^{7,2′-O})Gppp(m^{2′-O})N₁pN₂pN₃, (m₂^{7,3′-O})Gppp(m^{2′-O}) N₁pN₂N₃), wherein N₁, N₂, and N₃ are as defined and described herein. In some embodiments, a 5′ cap is a tetranucleotide Cap2 structure (e.g., (m⁷)Gppp(m^{2′-O})N₁p(m^{2′-O})N₂pN₃, (m₂^{7,2′-O})Gppp(m^{2′-O})N₁p(m^{2′-O})N₂pN₃, (m₂^{7,3′-O})Gppp(m^{2′-O})N₁p(m^{2′-O})N₂pN₃), wherein N₁, N₂, and N₃ are as defined and described herein. In some embodiments, a 5′ cap is selected from the group consisting of (m₂^{7,3′-O})Gppp(m^{2′-O})Ap(m^{2′-O})GpG, (m₂^{7,3′-O})Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, a 5′ cap is (m₂^{7,3′-O})Gppp(m^{2′-O})Ap(m^{2′-O})GpG, having a structure:

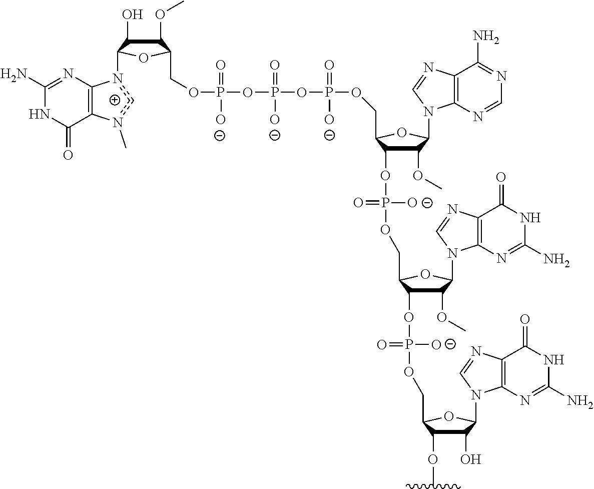

or a salt thereof.

In some embodiments, a 5′ cap is (m₂^{7,3′-O})Gppp(m^{2′-O})Gp(m^{2′-O}) GPC, having a structure:

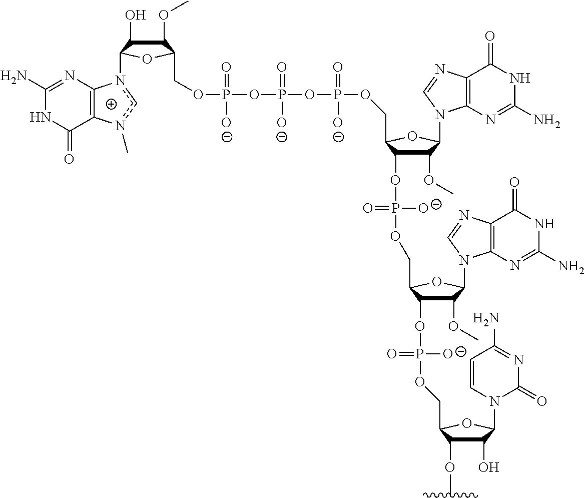

or a salt thereof.

In some embodiments, a 5′ cap is (m⁷)Gppp(m^{2′-O})Ap(m^{2′-O})UpA, having a structure:

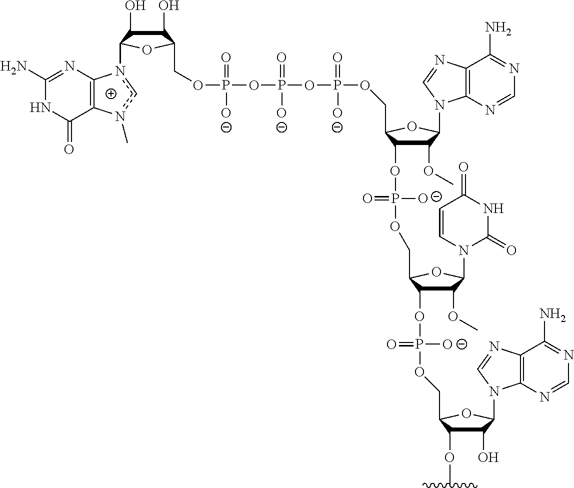

or a salt thereof.

In some embodiments, a 5′ cap is (m⁷)Gppp(m^{2′-O})Ap(m^{2′-O})GpG, having a structure:

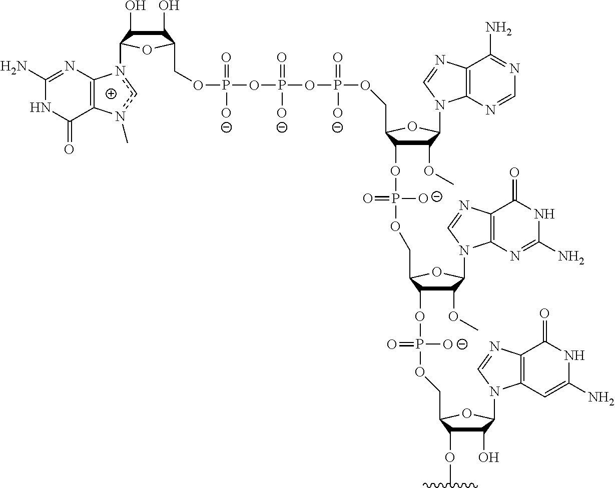

or a salt thereof.

In some embodiments, mRNA comprises a cap0, cap1, or cap2, preferably cap1 or cap2, more preferably cap1. According to the present disclosure, the term “cap0” comprises the structure “m7GpppN”, wherein N is any nucleoside bearing an OH moiety at position 2′. According to the present disclosure, the term “cap1” comprises the structure “m⁷GpppNm”, wherein Nm is any nucleoside bearing an OCH₃ moiety at position 2′. According to the present disclosure, the term “cap2” comprises the structure “m⁷GpppNmNm”, wherein each Nm is independently any nucleoside bearing an OCH₃ moiety at position 2′.

In some embodiments, the building block cap for RNA is m₂^{7,3′-O}Gppp(m₁^{2′-O})ApG (also sometimes referred to as m₂^{7,3′ O}G(5′)ppp(5′)m^{2′-O}ApG), which has the following structure:

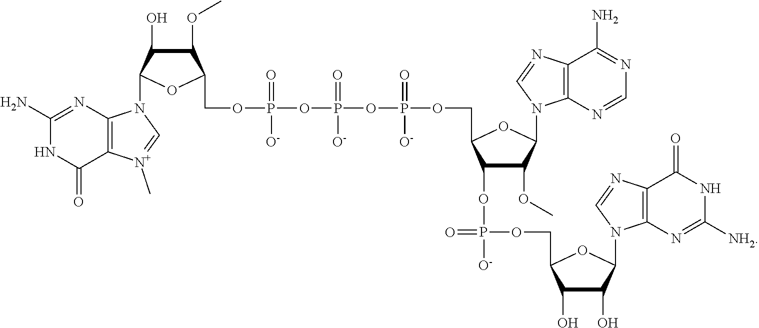

Below is an exemplary Cap1 RNA, which comprises RNA and m₂^{7,3′ O}G(5′)ppp(5′)m^{2′-O}ApG:

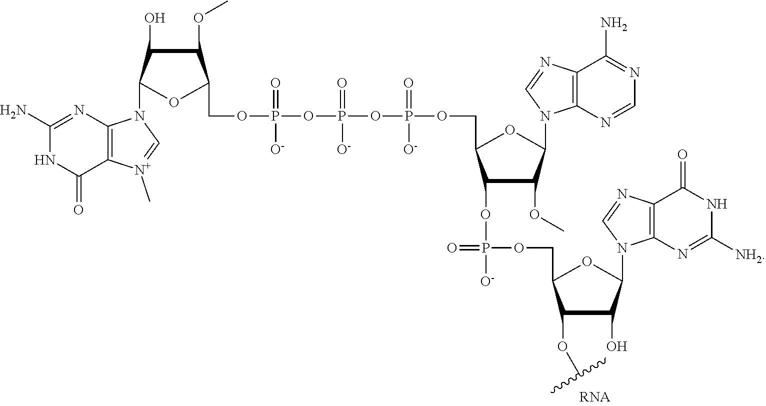

Below is another exemplary Cap1 RNA (no cap analog):

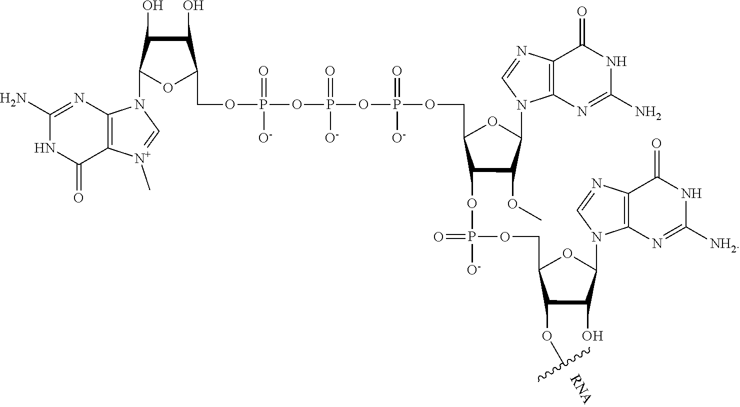

In some embodiments, RNA is modified with “Cap0” structures using, in one embodiment, the cap analog anti-reverse cap (ARCA Cap (m₂^{7,3′ O}G(5′)ppp(5′)G)) with the structure:

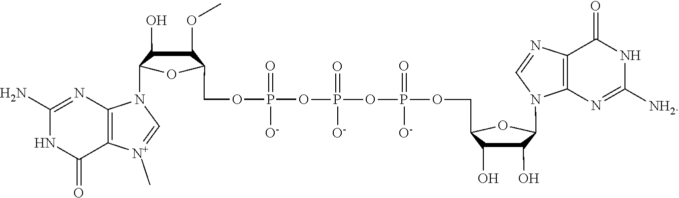

Below is an exemplary Cap0 RNA comprising RNA and m₂^{7,3′ O}G(5′)ppp(5′)G:

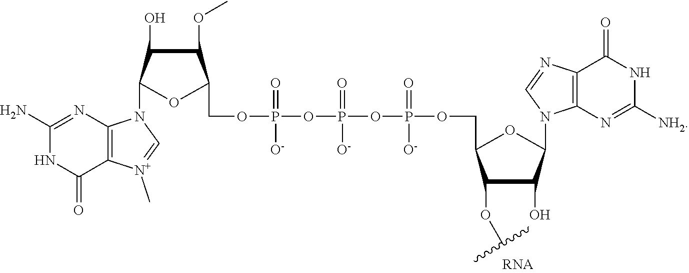

In some embodiments, the “Cap0” structures are generated using the cap analog Beta-S-ARCA (m₂^{7,2′ O}G(5′)ppSp(5′)G) with the structure:

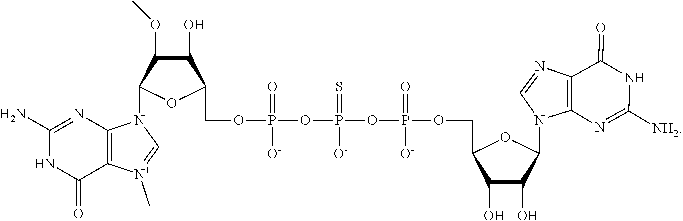

Below is an exemplary Cap0 RNA comprising Beta-S-ARCA (m₂^{7,2′ O}G(5′)ppSp(5′)G) and RNA:

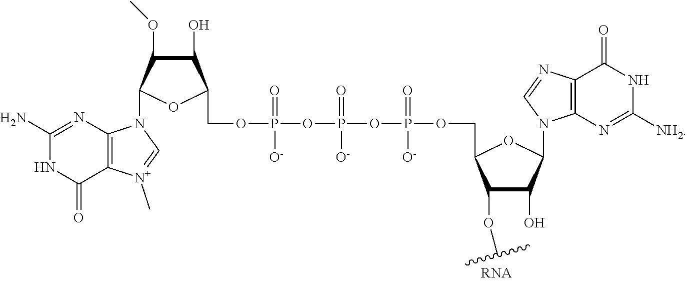

The “D1” diastereomer of beta-S-ARCA or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time (cf., WO 2011/015347, herein incorporated by reference).

A particularly preferred cap is beta-S-ARCA(D1) (m₂^{7,2′-O}GppSpG) or m₂^{7,3′-O}Gppp(m₁^{2′-O})ApG.

In some embodiments, an RNA comprises a 5′ cap, a cap proximal sequence (e.g., a sequence at the 5′ terminus of the RNA), or a combination thereof that is disclosed in WO2023/073190A1 or WO2021/214204, the contents of which are incorporated by reference herein in their entirety. For example, in some embodiments, an RNA comprises AGAAU at its 5′ terminus. In some embodiments, an RNA comprises AGCAC at its 5′ terminus.

### 5′ UTR and 3′ UTR

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end of an RNA, upstream of the start codon of a protein-encoding region. A 5′-UTR is downstream of the 5′-cap (if present), e.g. directly adjacent to the 5′-cap. A 3′-UTR, if present, is located at the 3′ end of an RNA, downstream of the termination codon of a protein-encoding region, but the term “3′-UTR” preferably does not include the poly(A) sequence. Thus, the 3′-UTR is upstream of the poly(A) sequence (if present), e.g. directly adjacent to the poly(A) sequence.

Exemplary 5′ UTRs include a human alpha globin (hAg) 5′UTR or a fragment thereof, a TEV 5′ UTR or a fragment thereof, a HSP70 5′ UTR or a fragment thereof, or a c-June 5′ UTR or a fragment thereof.

In some embodiments, an RNA disclosed herein comprises a hAg 5′ UTR sequence or a fragment thereof.

In some embodiments, RNA comprises a 5′-UTR comprising the nucleotide sequence of SEQ ID NO: 12, or a nucleotide sequence having at least 99%, 98%, 97%, 96%, 95%, 90%, 85%, or 80% identity to the nucleotide sequence of SEQ ID NO: 12.

In some embodiments, an RNA disclosed herein comprises a 3′ UTR comprising a first sequence from the amino terminal enhancer of split (AES) messenger RNA (an “F element”) and/or a second sequence from the mitochondrial encoded 12S ribosomal RNA (an “I element”).

In some embodiments, RNA comprises a 3′-UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

A particularly preferred 5′-UTR comprises the nucleotide sequence of SEQ ID NO: 12. A particularly preferred 3′-UTR comprises the nucleotide sequence of SEQ ID NO: 13.

In some embodiments, a 3′ UTR comprises a sequence that is at least 80% identical to SEQ ID NO: 13, which is immediately adjacent to and downstream of a sequence encoding an antigenic polypeptide. In some embodiments, a 3′ UTR comprises a sequence that is at least 80% identical to SEQ ID NO: 13, and comprises an intervening sequence between the sequence encoding an antigenic polypeptide and the sequence that is at least 80% identical to SEQ ID NO: 13. In some embodiments, the intervening sequence between the sequence encoding an antigenic polypeptide and the sequence that is at least 80% identical to SEQ ID NO: 13, comprises, in the 5′ to 3′ direction, the sequence CUCGAG. In some embodiments, the intervening sequence between the sequence encoding an antigenic polypeptide and the sequence that is at least 80% identical to SEQ ID NO: 13, comprises, in the 5′ to 3′ direction, the sequence GGAUCCGAU.

In some embodiments, a 3′ UTR is adjacent to (e.g., immediately adjacent to and upstream of) a polyA sequence that is at least 80% identical to SEQ ID NO: 14. In some embodiments, a 3′ UTR comprises an intervening sequence between the sequence that is at least 80% identical to SEQ ID NO: 13 and a polyA tail (e.g., a sequence that is at least 80% identical to SEQ ID NO: 14). In some embodiments, the intervening sequence between the sequence that is at least 80% identical to SEQ ID NO: 13 and the polyA tail comprises, in the 5′ to 3′ direction, the sequence CUXGAGCUAGC (SEQ ID NO: 176), where X is G, C, A, or U. In some embodiments, the intervening sequence between the sequence that is at least 80% identical to SEQ ID NO: 13 and the polyA tail comprises, in the 5′ to 3′ direction, the sequence GAGACCUGGUCCAGAGUCGCUAGCCGCGUCGCU (SEQ ID NO: 177).

In some embodiments, an RNA comprises non-coding elements as described in WO2021/213924, WO2023/285560A1, or WO2021/214204, the contents of each of which is incorporated by reference herein in their entirety.

### Poly(A) Sequence

In some embodiments, an RNA according to the present disclosure comprises a 3′-poly(A) sequence.

As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3′-UTR in an RNA described herein. An uninterrupted poly(A) sequence is characterized by consecutive adenylate residues. In nature, an uninterrupted poly(A) sequence is typical. RNAs disclosed herein can have a poly(A) sequence attached to the free 3′-end of an RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

The poly(A) sequence may be of any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly(A) sequence contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a poly(A) sequence included in an RNA described herein is a interrupted poly(A) sequence, e.g., as described in WO2016/005324, the entire content of which is incorporated herein by reference for purposes described herein. In some embodiments, a poly(A) sequence comprises a stretch of at least 20 adenosine residues (including, e.g., at least 30, at least 40, at least 50, at least 60, at least 70, or more adenosine residues), followed by a linker sequence (e.g., in some embodiments comprising non-A nucleotides) and another stretch of at least 20 adenosine residues (including, e.g., at least 30, at least 40, at least 50, at least 60, at least 70, or more adenosine residues). In some embodiments, such a linker sequence may be 3-50 nucleotides in length, or 5-25 nucleotides in length, or 10-15 nucleotides in length. In some embodiments, a poly(A) sequence comprises a stretch of about 30 adenosine residues, followed by a linker sequence having a length of about 5-15 nucleotides (e.g., in some embodiments comprising non-A nucleotides) and another stretch of about 70 adenosine residues.

In some embodiments, RNA comprises a poly(A) sequence comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

A particularly preferred poly(A) sequence comprises the nucleotide sequence of SEQ ID NO: 14.

According to the present disclosure, vaccine antigen is preferably administered as single-stranded, 5′-capped mRNA that is translated into the respective protein upon entering cells of a subject being administered the mRNA. Preferably, an RNA contains structural elements optimized for maximal efficacy of the RNA with respect to stability and translational efficiency (5′-cap, 5′-UTR, 3′-UTR, poly(A) sequence).

In one embodiment, beta-S-ARCA(D1) is utilized as specific capping structure at the 5′-end of an RNA. In one embodiment, m₂^{7,3′-O}Gppp(m₁^{2′-O})ApG is utilized as specific capping structure at the 5′-end of an RNA. In one embodiment, the 5′-UTR sequence is derived from the human alpha-globin mRNA and optionally has an optimized ‘Kozak sequence’ to increase translational efficiency. In one embodiment, a combination of two sequence elements (FI element) derived from the “amino terminal enhancer of split” (AES) mRNA (called F) and the mitochondrial encoded 12S ribosomal RNA (called I) are placed between the coding sequence and the poly(A) sequence to assure higher maximum protein levels and prolonged persistence of the mRNA. In one embodiment, two re-iterated 3′-UTRs derived from the human beta-globin mRNA are placed between the coding sequence and the poly(A) sequence to assure higher maximum protein levels and prolonged persistence of the mRNA. In one embodiment, a poly(A) sequence measuring 110 nucleotides in length, consisting of a stretch of 30 adenosine residues (SEQ ID NO: 174), followed by a 10 nucleotide linker sequence and another 70 adenosine residues (SEQ ID NO: 175) is used. This poly(A) sequence was designed to enhance RNA stability and translational efficiency.

In one embodiment of all aspects of the present disclosure, RNA encoding a vaccine antigen is expressed in cells of the subject treated to provide the vaccine antigen. In one embodiment of all aspects of the present disclosure, an RNA is transiently expressed in cells of the subject. In one embodiment of all aspects of the present disclosure, an RNA is in vitro transcribed RNA. In one embodiment of all aspects of the present disclosure, expression of a vaccine antigen encoded by an RNA is at the cell surface. In one embodiment of all aspects of the present disclosure, a vaccine antigen is expressed and presented in the context of MHC. In one embodiment of all aspects of the present disclosure, expression of a vaccine antigen is into the extracellular space, i.e., the vaccine antigen is secreted.

In the context of the present disclosure, the term “transcription” relates to a process, wherein the genetic code in a DNA sequence is transcribed into RNA. Subsequently, an RNA may be translated into peptide or protein.

According to the present disclosure, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present disclosure encompassed by the term “vector”. According to the present disclosure, an RNA used in the present disclosure preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the present disclosure is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

With respect to RNA, the term “expression” or “translation” relates to the process in the ribosomes of a cell by which a strand of mRNA directs the assembly of a sequence of amino acids to make a peptide or protein.

In one embodiment, after administration of RNA described herein, e.g., formulated as RNA lipid particles, at least a portion of the RNA is delivered to a target cell. In one embodiment, at least a portion of the RNA is delivered to the cytosol of the target cell. In one embodiment, the RNA is translated by the target cell to produce the peptide or protein it encodes. In one embodiment, the target cell is a spleen cell. In one embodiment, the target cell is an antigen presenting cell such as a professional antigen presenting cell in the spleen. In one embodiment, the target cell is a dendritic cell or macrophage. RNA particles such as RNA lipid particles described herein may be used for delivering RNA to such target cell. Accordingly, the present disclosure also relates to a method for delivering RNA to a target cell in a subject comprising the administration of the RNA particles described herein to the subject. In one embodiment, RNA is delivered to the cytosol of the target cell. In one embodiment, RNA is translated by the target cell to produce the peptide or protein encoded by the RNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In one embodiment, RNA encoding a vaccine antigen to be administered according to the present disclosure is non-immunogenic. RNA encoding immunostimulant may be administered according to the present disclosure to provide an adjuvant effect. RNA encoding immunostimulant may be standard RNA or non-immunogenic RNA.

The term “non-immunogenic RNA” as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In one preferred embodiment, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the non-immunogenic RNA non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In one embodiment, the modified nucleosides comprises a replacement of one or more uridines with a nucleoside comprising a modified nucleobase. In one embodiment, the modified nucleobase is a modified uracil. In one embodiment, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (Tm⁵U), 1-taurinomethyl-pseudouridine, 5-taurinomethyl-2-thio-uridine (Tm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ ψ), 5-(isopentenylaminomethyl) uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine. In one particularly preferred embodiment, the nucleoside comprising a modified nucleobase is pseudouridine (ψ), N1-methyl-pseudouridine (m1ψ) or 5-methyl-uridine (m5U), in particular N1-methyl-pseudouridine.

In one embodiment, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In one embodiment, an RNA preparation is contacted with a cellulose material and the ssRNA is separated from the cellulose material under conditions which allow binding of dsRNA to the cellulose material and do not allow binding of ssRNA to the cellulose material.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In one embodiment, the removal of dsRNA from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In one embodiment, the non-immunogenic RNA is free or essentially free of dsRNA. In some embodiments, the non-immunogenic RNA composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In one embodiment, the non-immunogenic RNA is translated in a cell more efficiently than standard RNA with the same sequence. In one embodiment, translation is enhanced by a factor of 2-fold relative to its unmodified counterpart. In one embodiment, translation is enhanced by a 3-fold factor. In one embodiment, translation is enhanced by a 4-fold factor. In one embodiment, translation is enhanced by a 5-fold factor. In one embodiment, translation is enhanced by a 6-fold factor. In one embodiment, translation is enhanced by a 7-fold factor. In one embodiment, translation is enhanced by an 8-fold factor. In one embodiment, translation is enhanced by a 9-fold factor. In one embodiment, translation is enhanced by a 10-fold factor. In one embodiment, translation is enhanced by a 15-fold factor. In one embodiment, translation is enhanced by a 20-fold factor. In one embodiment, translation is enhanced by a 50-fold factor. In one embodiment, translation is enhanced by a 100-fold factor. In one embodiment, translation is enhanced by a 200-fold factor. In one embodiment, translation is enhanced by a 500-fold factor. In one embodiment, translation is enhanced by a 1000-fold factor. In one embodiment, translation is enhanced by a 2000-fold factor. In one embodiment, the factor is 10-1000-fold. In one embodiment, the factor is 10-100-fold. In one embodiment, the factor is 10-200-fold. In one embodiment, the factor is 10-300-fold. In one embodiment, the factor is 10-500-fold. In one embodiment, the factor is 20-1000-fold. In one embodiment, the factor is 30-1000-fold. In one embodiment, the factor is 50-1000-fold. In one embodiment, the factor is 100-1000-fold. In one embodiment, the factor is 200-1000-fold. In one embodiment, translation is enhanced by any other significant amount or range of amounts.

In one embodiment, the non-immunogenic RNA exhibits significantly less innate immunogenicity than standard RNA with the same sequence. In one embodiment, the non-immunogenic RNA exhibits an innate immune response that is 2-fold less than its unmodified counterpart. In one embodiment, innate immunogenicity is reduced by a 3-fold factor. In one embodiment, innate immunogenicity is reduced by a 4-fold factor. In one embodiment, innate immunogenicity is reduced by a 5-fold factor. In one embodiment, innate immunogenicity is reduced by a 6-fold factor. In one embodiment, innate immunogenicity is reduced by a 7-fold factor. In one embodiment, innate immunogenicity is reduced by a 8-fold factor. In one embodiment, innate immunogenicity is reduced by a 9-fold factor. In one embodiment, innate immunogenicity is reduced by a 10-fold factor. In one embodiment, innate immunogenicity is reduced by a 15-fold factor. In one embodiment, innate immunogenicity is reduced by a 20-fold factor. In one embodiment, innate immunogenicity is reduced by a 50-fold factor. In one embodiment, innate immunogenicity is reduced by a 100-fold factor. In one embodiment, innate immunogenicity is reduced by a 200-fold factor. In one embodiment, innate immunogenicity is reduced by a 500-fold factor. In one embodiment, innate immunogenicity is reduced by a 1000-fold factor. In one embodiment, innate immunogenicity is reduced by a 2000-fold factor.

The term “exhibits significantly less innate immunogenicity” refers to a detectable decrease in innate immunogenicity. In one embodiment, the term refers to a decrease such that an effective amount of the non-immunogenic RNA can be administered without triggering a detectable innate immune response. In one embodiment, the term refers to a decrease such that the non-immunogenic RNA can be repeatedly administered without eliciting an innate immune response sufficient to detectably reduce production of the protein encoded by the non-immunogenic RNA. In one embodiment, the decrease is such that the non-immunogenic RNA can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked,” “fused”, or “fusion” are used interchangeably. These terms refer to the joining together of two or more elements or components or domains.

### Codon-Optimization/Increase in G/C Content

In some embodiment, an antigenic polypeptide associated with an infectious agent is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present disclosure, coding regions are preferably codon-optimized for optimal expression in a subject to be treated using RNA molecules described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments of the present disclosure, the guanosine/cytosine (G/C) content of the coding region of RNA described herein is increased compared to the G/C content of the corresponding coding sequence of the wild type RNA, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favourable codons for the stability can be determined (so-called alternative codon usage). Depending on the amino acid to be encoded by an RNA, there are various possibilities for modification of an RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of an RNA described herein is increased by at least 10%, at least 20%, at least 30%, at least 40%, at least 50%, at least 55%, or even more compared to the G/C content of the coding region of the wild type RNA. In some embodiments, G/C content of a coding region is increased by about 10% to about 60% (e.g., by about 20% to about 60%, about 30% to about 60%, about 40% to about 60%, about 50% to about 60%, or by about 51%, about 52%, about 53%, about 54%, about 55%, about 56%, about 57%, about 58%, about 59%, or about 60%) compared to the G/C content of the coding region of the wild type RNA.

In some embodiments, RNA disclosed herein comprises a sequence disclosed herein (e.g., SEQ ID NO: 9), that has been modified to encode one or more mutations characteristic of a SARS-CoV-2 variant (e.g., ones described herein including but not limited to a BA.2 or a BA.4/5 Omicron variant). In some embodiments, RNA can be modified to encode one or more mutations characteristic of a SARS-CoV-2 variant by making as few nucleotide changes as possible. In some embodiments, RNA can be modified to encode one or more mutations that are characteristic of a SARS-CoV-2 variant by introducing mutations that result in high codon-optimization and/or increased G/C content. In some embodiments, one or more mutations characteristic of a SARS-CoV-2 variant are introduced onto a full-length S protein (e.g., an S protein comprising SEQ ID NO: 1). In some embodiments one or more mutations characteristic of a SARS-CoV-2 variant are introduced onto a full-length S protein having one or more proline mutations that increase stability of a prefusion confirmation. For example, in some embodiments, proline substitutions are made at positions corresponding to positions 986 and 987 of SEQ ID NO: 1. In some embodiments, at least 4 proline substitutions are made. In some embodiments, at least four of such proline mutations include mutations at positions corresponding to residues 817, 892, 899, and 942 of SEQ ID NO: 1, e.g., as described in WO 2021243122 A2, the entire contents of which are incorporated herein by reference in its entirety. In some embodiments, such a SARS-CoV-2 S protein comprising proline substitutions at positions corresponding to residues 817, 892, 899, and 942 of SEQ ID NO: 1, may further comprise proline substitutions at positions corresponding to residues 986 and 987 of SEQ ID NO: 1. In some embodiments, one or more mutations characteristic of a SARS-CoV-2 variant are introduced onto an immunogenic fragment of an S protein (e.g., the RBD of SEQ ID NO: 1).

### Embodiments of Administered RNAs

In some embodiments, the present disclosure provides a composition comprising (i) one or more first RNAs, each comprising a nucleotide sequence encoding an antigenic polypeptide associated with a first infectious agent, and (ii) one or more second RNAs, each comprising a nucleotide sequence encoding an antigenic polypeptide associated with a second infectious agent, wherein the first infectious agent is different from the second infectious agent. In some embodiments, the first infectious agent and second infectious agent can each be independently an infectious bacterial agent and/or an infectious viral agent. In some embodiments, the first infectious agent and second infectious agents can each be independently selected from coronaviruses (including, e.g., alphacoronavirus, betacoronavirus, gammacoronavirus, deltacoronavirus), influenza viruses (e.g., Type A, B, C or D), pneumoviridae (e.g., respiratory syncytial viruses), and combinations thereof.

In some embodiments, the composition comprises at least one first RNA comprising an open reading frame encoding a coronavirus antigen and at least one second RNA comprising an open reading frame encoding a non-coronavirus infectious disease antigen. In some embodiments, the composition comprises at least one first RNA comprising an open reading frame encoding a coronavirus antigen and at least one second RNA comprising an open reading frame encoding an influenza antigen. Exemplary influenza antigens include hemagglutinin (HA) and neuraminidase (NA) and immunogenic fragments thereof. In some embodiments, the composition comprises at least one first RNA encoding a polypeptide that comprises at least an immunogenic portion of a SARS-CoV-2 S protein (e.g., a RBD portion of a SARS-CoV-2 S protein). In some embodiments, the composition comprises at least one first RNA encoding a full-length S protein. In some embodiments, the composition comprises at least one first RNA encoding a stabilized prefusion S protein. In some embodiments, the composition comprises at least one second RNA comprising an open reading frame encoding a polypeptide that comprises at least a portion of an influenza HA protein.

In some embodiments, the composition comprises at least one first RNA comprising an open reading frame encoding a polypeptide that comprises at least a portion of a SARS-CoV-2 S protein. In some embodiments, the composition comprises at least one second RNA comprising an open reading frame encoding a polypeptide that comprises at least a portion of an influenza HA protein. Each RNA in such compositions is suitable for intracellular expression of an encoded polypeptide. In some embodiments, such an encoded polypeptide comprises a sequence corresponding to the complete S protein. In some embodiments, such an encoded polypeptide does not comprise a sequence corresponding to the complete S protein. In some embodiments, the encoded polypeptide comprises a sequence that corresponds to the receptor binding domain (RBD). In some embodiments, the encoded polypeptide comprises a sequence that corresponds to the RBD, and further comprises a trimerization domain (e.g., a trimerization domain as disclosed herein, such as a fibritin domain). In some embodiments an RBD comprises a signaling domain (e.g., a signaling domain as disclosed herein). In some embodiments an RBD comprises a transmembrane domain (e.g., a transmembrane domain as disclosed herein). In some embodiments, an RBD comprises a signaling domain and a trimerization domain. In some embodiments, an RBD comprises a signaling domain, a trimerization domain, and transmembrane domain. In some embodiments, such an encoded polypeptide comprises a sequence corresponding to the complete HA protein.

In some embodiments, the encoded polypeptide comprises a sequence that corresponds to two receptor binding domains. In some embodiments, the encoded polypeptide comprises a sequence that corresponds to two receptor binding domains in tandem in an amino acid chain, e.g., as disclosed in Dai, Lianpan, et al. “A universal design of betacoronavirus vaccines against COVID-19, MERS, and SARS,” Cell 182.3 (2020): 722-733, the contents of which are incorporated by reference herein in their entirety.

In some embodiments, a SARS-CoV-2 S protein, or an immunogenic fragment thereof comprises one or more mutations to alter or remove a glycosylation site, e.g., as described in WO2022221835A2, US20220323574A1, or WO2022195351A1.

In some embodiments, compositions or medical preparations described herein comprise RNA encoding an amino acid sequence comprising SARS-CoV-2 S protein, an immunogenic variant thereof, or an immunogenic fragment of the SARS-CoV-2 S protein or the immunogenic variant thereof. Likewise, methods described herein comprise administration of such RNA.

The active platform for use herein is based on an antigen-coding RNA vaccine to induce robust neutralising antibodies and accompanying/concomitant T cell response to achieve protective immunization with preferably minimal vaccine doses. RNA administered is preferably in-vitro transcribed RNA.

Three different RNA platforms are particularly preferred, namely non-modified uridine containing mRNA (uRNA), nucleoside modified mRNA (modRNA) and self-amplifying RNA (saRNA). In one particularly preferred embodiment, RNA is in vitro transcribed RNA. In some embodiments, uRNA is mRNA. In some embodiments, modRNA is mRNA. In the following, embodiments of these three different RNA platforms are described, wherein certain terms used when describing elements thereof have the following meanings:

S1S2 protein/S1S2 RBD: Sequences encoding the respective antigen of SARS-CoV-2.

nsP1, nsP2, nsP3, and nsP4: Wildtype sequences encoding the Venezuelan equine encephalitis virus (VEEV) RNA-dependent RNA polymerase replicase and a subgenomic promotor plus conserved sequence elements supporting replication and translation.

virUTR: Viral untranslated region encoding parts of the subgenomic promotor as well as replication and translation supporting sequence elements.

hAg-Kozak: 5′-UTR sequence of the human alpha-globin mRNA with an optimized ‘Kozak sequence’ to increase translational efficiency.

Sec: Sec corresponds to a secretory signal peptide (sec), which guides translocation of the nascent polypeptide chain into the endoplasmic reticulum. In some embodiments, such a secretory signal peptide includes the intrinsic S1S2 secretory signal peptide. In some embodiments, such a secretory signal peptide is a secretory signal peptide from a non-S1S2 protein. For example, an immunoglobulin secretory signal peptide (aa 1-22), an HSV-1 gD signal peptide (MGGAAARLGAVILFVVIVGLHGVRSKY (SEQ ID NO: 110)), an HSV-2 gD signal peptide (MGRLTSGVGTAALLVVAVGLRVVCA (SEQ ID NO: 111)); a human SPARC signal peptide, a human insulin isoform 1 signal peptide, a human albumin signal peptide, or any other signal peptide described herein.

Glycine-serine linker (GS): Sequences coding for short linker peptides predominantly consisting of the amino acids glycine (G) and serine(S), as commonly used for fusion proteins.

Fibritin: Partial sequence of T4 fibritin (foldon), used as artificial trimerization domain.

TM: TM sequence corresponds to the transmembrane part of a protein. A transmembrane domain can be N-terminal, C-terminal, or internal to an encoded polypeptide. A coding sequence of a transmembrane element is typically placed in frame (i.e., in the same reading frame), 5′, 3′, or internal to coding sequences of sequences (e.g., sequences encoding polypeptide(s)) with which it is to be linked. In some embodiments, a transmembrane domain comprises or is a transmembrane domain of Hemagglutinin (HA) of Influenza virus, Env of HIV-1, equine infectious anaemia virus (EIAV), murine leukaemia virus (MLV), mouse mammary tumor virus, G protein of vesicular stomatitis virus (VSV), Rabies virus, or a seven transmembrane domain receptor. In some embodiments, the transmembrane part of a protein is from the S1S2 protein.

FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.

A30L70: A poly(A)-tail measuring 110 nucleotides in length, consisting of a stretch of 30 adenosine residues (SEQ ID NO: 174), followed by a 10 nucleotide linker sequence and another 70 adenosine residues (SEQ ID NO: 175) designed to enhance RNA stability and translational efficiency in dendritic cells.

In some embodiments, vaccine RNA described herein may comprise, from 5′ to 3′, one of the following structures:
Cap-5′-UTR-Vaccine Antigen-Encoding Sequence-3′-UTR-Poly(A)
   or
Cap-hAg-Kozak-Vaccine Antigen-Encoding Sequence-FI-A30L70.

In some embodiments, a vaccine antigen described herein may comprise a full-length S protein or an immunogenic fragment thereof (e.g., RBD). In some embodiments where a vaccine antigen comprises a full-length S protein, its secretory signal peptide and/or transmembrane domain may be replaced by a heterologous secretory signal peptide (e.g., as described herein) and/or a heterologous transmembrane domain (e.g., as described herein).

In some embodiments, a vaccine antigen described herein may comprise, from N-terminus to C-terminus, one of the following structures:
Signal Sequence-RBD-Trimerization Domain
   or
Signal Sequence-RBD-Trimerization Domain-Transmembrane Domain.

RBD and Trimerization Domain may be separated by a linker, in particular a GS linker such as a linker having the amino acid sequence GSPGSGSGS (SEQ ID NO: 33). Trimerization Domain and Transmembrane Domain may be separated by a linker, in particular a GS linker such as a linker having the amino acid sequence GSGSGS (SEQ ID NO: 34).

Signal Sequence may be a signal sequence as described herein. RBD may be a RBD domain as described herein. Trimerization Domain may be a trimerization domain as described herein. Transmembrane Domain may be a transmembrane domain as described herein.

In one embodiment,
Signal sequence comprises the amino acid sequence of amino acids 1 to 16 or 1 to 19 of SEQ ID NO: 1 or the amino acid sequence of amino acids 1 to 22 of SEQ ID NO: 31, or an amino acid sequence having at least 99%, 98%, 97%, 96%, 95%, 90%, 85%, or 80% identity to this amino acid sequence,
RBD comprises the amino acid sequence of amino acids 327 to 528 of SEQ ID NO: 1, or an amino acid sequence having at least 99%, 98%, 97%, 96%, 95%, 90%, 85%, or 80% identity to this amino acid sequence,
Trimerization Domain comprises the amino acid sequence of amino acids 3 to 29 of SEQ ID NO: 10 or the amino acid sequence of SEQ ID NO: 10, or an amino acid sequence having at least 99%, 98%, 97%, 96%, 95%, 90%, 85%, or 80% identity to this amino acid sequence; and
Transmembrane Domain comprises the amino acid sequence of amino acids 1207 to 1254 of SEQ ID NO: 1, or an amino acid sequence having at least 99%, 98%, 97%, 96%, 95%, 90%, 85%, or 80% identity to this amino acid sequence.

In one embodiment,
Signal sequence comprises the amino acid sequence of amino acids 1 to 16 or 1 to 19 of SEQ ID NO: 1 or the amino acid sequence of amino acids 1 to 22 of SEQ ID NO: 31,
RBD comprises the amino acid sequence of amino acids 327 to 528 of SEQ ID NO: 1,
Trimerization Domain comprises the amino acid sequence of amino acids 3 to 29 of SEQ ID NO: 10 or the amino acid sequence of SEQ ID NO: 10; and
Transmembrane Domain comprises the amino acid sequence of amino acids 1207 to 1254 of SEQ ID NO: 1.

In some embodiments, an RNA polynucleotide comprising a sequence encoding a vaccine antigen (e.g., a SARS-CoV-2 S protein, an immunogenic variant thereof, an immunogenic fragment of the SARS-CoV-2 S protein or the immunogenic variant thereof or an HA protein or an immunogenic variant thereof) or comprising an open reading frame encoding a vaccine antigen (e.g., a SARS-CoV-2 S protein, an HA protein, immunogenic variants thereof, or an immunogenic fragments of the SARS-CoV-2 S protein, HA protein or immunogenic variants thereof) such as the nucleotide sequence of SEQ ID NO: 50 or the nucleotide sequence of SEQ ID NO: 53, a variant or fragment thereof, further comprises a 5′ cap, e.g., a 5′ cap comprising a Cap1 structure, a 5′ UTR sequence, e.g., a 5′ UTR sequence comprising the nucleotide sequence of SEQ ID NO: 12, a 3′ UTR sequence, e.g., a 3′ UTR sequence comprising the nucleotide sequence of SEQ ID NO: 13, and polyA sequence, e.g., a polyA sequence comprising the nucleotide sequence of SEQ ID NO: 14. In some embodiments, an RNA polynucleotide is formulated in a composition comprising ((4-hydroxybutyl)azanediyl)bis(hexane-6,1-diyl)bis(2-hexyldecanoate), cholesterol, distearoylphosphatidylcholine, and (2-[(polyethylene glycol)-2000]-N,N-ditetradecylacetamide).

RNA described herein or RNA encoding a vaccine antigen described herein may be non-modified uridine containing mRNA (uRNA), nucleoside modified mRNA (modRNA) or self-amplifying RNA (saRNA). In one embodiment, an RNA described herein or RNA encoding the vaccine antigen described herein is nucleoside modified mRNA (modRNA).

### Exemplary RNAs Encoding Variant Specific SARS-CoV-2 Antigens

In some embodiments, RNA disclosed herein encodes an S protein comprising one or more mutations that are characteristic of a SARS-CoV-2 variant. In some embodiments, RNA encodes a SARS-CoV-2 S protein comprising one or more mutations characteristic of an Alpha variant. In some embodiments, RNA encodes a SARS-CoV-2 S protein comprising one or more mutations characteristic of a Beta variant. In some embodiments, RNA encodes a SARS-CoV-2 S protein comprising one or more mutations characteristic of a Delta variant. In some embodiments, RNA encodes a SARS-CoV-2 S protein comprising one or more mutations characteristic of an Omicron variant (e.g., an S protein comprising one or more mutations characteristic of a BA.1, BA.2, or BA.4/5 Omicron variant). In some embodiments, RNA encodes a SARS-CoV-2 S protein comprising one or more mutations characteristic of an Omicron XBB variant (e.g., an S protein comprising one or more mutations characteristic of an XBB.1.5, XBB.1.16, XBB.2.3, or XBB.2.3.2 variant). In some embodiments, RNA encodes a SARS-CoV-2 S protein comprising one or more mutations characteristic of an BA.1 Omicron variant. In some embodiments, RNA encodes a SARS-CoV-2 S protein comprising one or more mutations characteristic of an BA.2 Omicron variant. In some embodiments, RNA encodes a SARS-CoV-2 S protein comprising one or more mutations characteristic of an BA.2.12.1 Omicron variant. In some embodiments, RNA encodes a SARS-CoV-2 S protein comprising one or more mutations characteristic of a BA.3 Omicron variant. In some embodiments, RNA encodes a SARS-CoV-2 S protein comprising one or more mutations characteristic of a BA.4 Omicron variant. In some embodiments, RNA encodes a SARS-CoV-2 S protein comprising one or more mutations characteristic of a BA.5 Omicron variant.

In some embodiments, the compositions disclosed herein comprise a bivalent SARS-CoV-2 vaccine. In some embodiments, the SARS-CoV-2 bivalent vaccine comprises an RNA encoding a SARS-CoV-2 S protein of a Wuhan strain and an RNA encoding a SARS-CoV-2 S protein associated with a variant that is predicted to be prevalent in a relevant jurisdiction. In some embodiments, the SARS-CoV-2 bivalent vaccine comprises two RNAs, each encoding an S protein of a SARS-CoV-2 variant that is predicted to be prevalent in a relevant jurisdiction. In some embodiments, compositions disclosed herein are updated annually, so as to encode SARS-CoV-2 S proteins of variants that are predicted to be most prevalent in a relevant jurisdiction.

### Exemplary RNAs Encoding Influenza Antigens

In some embodiments, RNA disclosed herein comprises a nucleotide sequence encoding an antigenic polypeptide associated with influenza. In some embodiments, the antigenic polypeptide associated with influenza is a hemagglutinin (HA) protein. In some embodiments, the antigenic polypeptide associated with influenza is a neuraminidase (NA) protein. In some embodiments, the antigenic polypeptide associated with influenza includes at least one conserved epitope (e.g., as described in WO2021202734A2; Freyn, Alec W., et al. “A multi-targeting, nucleoside-modified mRNA influenza virus vaccine provides broad protection in mice,” Molecular Therapy 28.7 (2020): 1569-1584; and/or Ekiert, Damian C., et al. “Antibody recognition of a highly conserved influenza virus epitope,” Science 324.5924 (2009): 246-251, the contents of each of which are incorporated by reference herein in their entirety).

In some embodiments, a composition comprises an RNA encoding an antigen (e.g., an HA protein) of an influenza virus that is recommended by a relevant health authority for inclusion in a seasonally-adapted vaccine (e.g., a cell-based, recombinant, or live attenuated virus). In some embodiments a composition comprises a plurality of RNAS, encoding antigens (e.g., HA proteins) of each influenza virus recommended by a relevant health authority for inclusion in a seasonally-adapted vaccine (e.g., a cell-based, recombinant, or live attenuated virus).

In some embodiments, the influenza virus is an influenza A, influenza B, or influenza C virus. In some embodiments, the influenza A virus is an H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, or H10N8 virus. In some embodiments, the influenza A virus is an H1N1, H3N2, H5N1, or H5N8 virus. In some embodiments, the influenza A virus is an H1N1 virus (e.g., A/Wisconsin/588/2019 or A/Sydney/5/2021). In some embodiments the influenza A virus is an H3N2 virus. In some embodiments the H3N2 virus is A/Cambodia/e0826360/2020 or A/Darwin/6/2021. In some embodiments, the influenza B virus is of a B/Yamagata or B/Victoria lineage. In some embodiments, the B/Victoria lineage influenza virus is B/Washington/02/2019. In some embodiments, the B/Victoria lineage virus is B/Austria/1359417/2021. In some embodiments, the B/Yamagata lineage influenza virus is B/Phuket/3073/2013. In some embodiments, a composition described herein comprises a multivalent influenza vaccine. In some embodiments, a multivalent influenza vaccine comprises 2 to 50 RNA distinct molecules (e.g., 2 to 40, 2 to 30, or 2 to 20 RNA molecules), each of which, in some embodiments, may encode a different antigenic polypeptide (or a different version of a particular antigenic polypeptide) associated with influenza, e.g., as described in Arevalo, Claudia P., et al. “A multivalent nucleoside-modified mRNA vaccine against all known influenza virus subtypes.” Science 378.6622 (2022): 899-904. In some embodiments, a composition described herein comprises a trivalent influenza vaccine. In some embodiments, a trivalent influenza vaccine comprises RNAs encoding an antigenic polypeptide associated with two type A viruses and one type B virus that are predicted to be prevalent in a relevant jurisdiction. In some embodiments, a composition described herein comprises a tetravalent influenza vaccine. In some embodiments, a tetravalent influenza vaccine comprises RNAs encoding an antigenic polypeptide associated with two type A viruses and two type B viruses that are predicted to be prevalent in a relevant jurisdiction. In some embodiments, a composition described herein comprises an octavalent influenza vaccine. In some embodiments, an octavalent influenza vaccine comprises RNAs encoding two antigenic polypeptides associated with each of two type A viruses and two type B viruses that are predicted to be prevalent in a relevant jurisdiction (e.g., an HA protein and an NA protein associated with each virus, or immunogenic fragments thereof). In some embodiments, a composition disclosed herein comprises a tetravalent influenza vaccine comprising an RNA comprising a nucleotide sequence encoding an HA protein associated with an H1N1 virus (e.g., A/Wisconsin/588/2019), an RNA comprising a nucleotide sequence encoding an HA protein associated with an H3N2 virus (e.g., A/Cambodia/e0826360/2020), an RNA comprising a nucleotide sequence encoding an HA protein associated with a B/Victoria lineage influenza virus (e.g., B/Washington/02/2019), and an HA protein associated with a B/Yamagata lineage influenza virus (e.g., B/Phuket/3073/2013). In some embodiments, such a composition comprising a tetravalent influenza vaccine further comprises a coronavirus vaccine. In some embodiments, such a coronavirus vaccine is a SARS-CoV-2 vaccine. In some embodiments, a SARS-CoV-2 vaccine is a bivalent SARS-CoV-2 vaccine (e.g., (i) a SARS-CoV-2 bivalent vaccine comprising an RNA encoding a SARS-CoV-2 S protein of a Wuhan strain and an RNA encoding a SARS-CoV-2 S protein comprising one or more mutations associated with a variant that is prevalent in relevant populations around the time of administration. For example, in some embodiments, a SARS-CoV-2 vaccine is a bivalent SARS-CoV-2 vaccine (e.g., (i) a SARS-CoV-2 bivalent vaccine comprising an RNA encoding a SARS-CoV-2 S protein of a Wuhan strain and an RNA encoding a SARS-CoV-2 S protein comprising one or more mutations associated with a BA.4/5 Omicron variant.

In some embodiments, a composition comprises a tetravalent influenza vaccine comprises RNA encoding an antigenic polypeptide associated with two type A viruses and two type B viruses that are predicted to be prevalent in a relevant jurisdiction. In some embodiments, a tetravalent influenza vaccine comprises RNA encoding an antigenic polypeptide associated with an H1N1 influenza virus, RNA encoding an antigenic polypeptide associated with an H3N2 influenza virus, RNA encoding an antigenic polypeptide associated with a Victoria lineage influenza virus, and RNA encoding an antigenic polypeptide associated with a Yamagata lineage influenza virus. In some embodiments, the tetravalent influenza vaccine comprises RNA associated with influenza types that are predicted to be prevalent in a relevant jurisdiction (e.g., HA polypeptides associated with the H1N1, H3N2, B/Victoria, and B/Yamagata influenza viruses that are predicted to be prevalent in a relevant jurisdiction).

In some embodiments, a composition comprises (i) a tetravalent influenza vaccine comprising RNA encoding HA polypeptides associated with influenza virus strains that are predicted to be most prevalent in a relevant jurisdiction and (ii) a bivalent SARS-CoV-2 vaccine comprising (a) RNA encoding a SARS-CoV-2 S protein of two variants that are predicted to be most prevalent in a relevant jurisdiction, or (b) RNA encoding a SARS-CoV-2 S protein of a Wuhan strain and RNA encoding a SARS-CoV-2 S protein of a variant that is predicted to be prevalent in a relevant jurisdiction.

In some embodiments, each of the RNAs in a composition disclosed herein encodes an antigenic polypeptide associated with an infectious agent that is predicted to be prevalent in a relevant jurisdiction. Such compositions can reduce the number of vaccinations needed.

Non-Modified Uridine Messenger RNA (uRNA)

In some embodiments, a non-modified uridine RNA is a messenger RNA. The active principle of non-modified messenger RNA (uRNA) drug substance is a single-stranded RNA (e.g., mRNA) that can be translated upon entering a cell. In addition to a sequence encoding a vaccine antigen (i.e. open reading frame), each uRNA preferably contains common structural elements optimized for maximal efficacy of an RNA with respect to stability and translational efficiency (5′-cap, 5′-UTR, 3′-UTR, poly(A)-tail). The preferred 5′ cap structure is beta-S-ARCA(D1) (m₂^{7,2′-O}GppSpG). The preferred 5′-UTR and 3′-UTR comprise the nucleotide sequence of SEQ ID NO: 12 and the nucleotide sequence of SEQ ID NO: 13, respectively. The preferred poly(A)-tail comprises the sequence of SEQ ID NO: 14.

Different embodiments of this platform are as follows:

### RBL063.1 (SEQ ID NO: 15; SEQ ID NO: 7)

Structure beta-S-ARCA(D1)-hAg-Kozak-S1S2-PP-FI-A30L70
Encoded antigen Viral spike protein (S1S2 protein) of the SARS-CoV-2 (S1S2 full-length protein, sequence variant)

### RBL063.2 (SEQ ID NO: 16; SEQ ID NO: 7)

Structure beta-S-ARCA(D1)-hAg-Kozak-S1S2-PP-FI-A30L70
Encoded antigen Viral spike protein (S1S2 protein) of the SARS-CoV-2 (S1S2 full-length protein, sequence variant)

### BNT162a1; RBL063.3 (SEQ ID NO: 17; SEQ ID NO: 5)

Structure beta-S-ARCA(D1)-hAg-Kozak-RBD-GS-Fibritin-FI-A30L70
Encoded antigen Viral spike protein (S protein) of the SARS-CoV-2 (partial sequence, Receptor Binding Domain (RBD) of S1S2 protein)

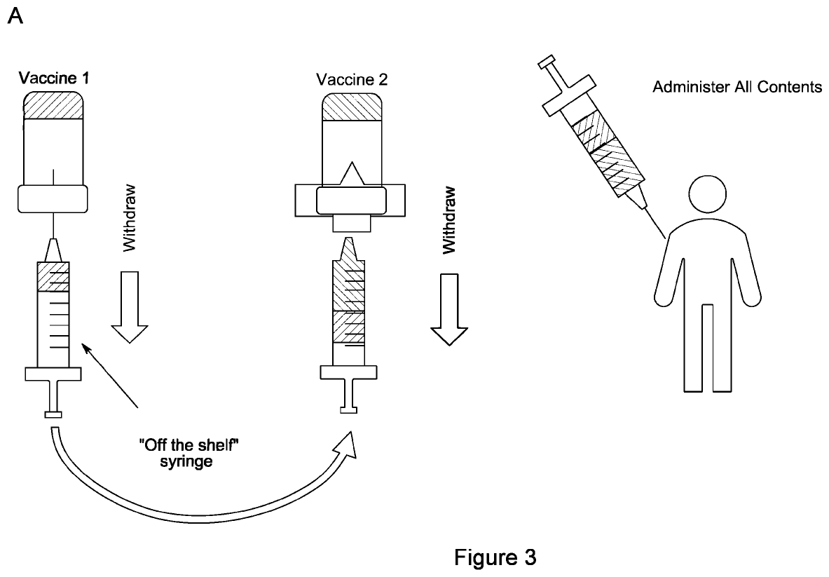

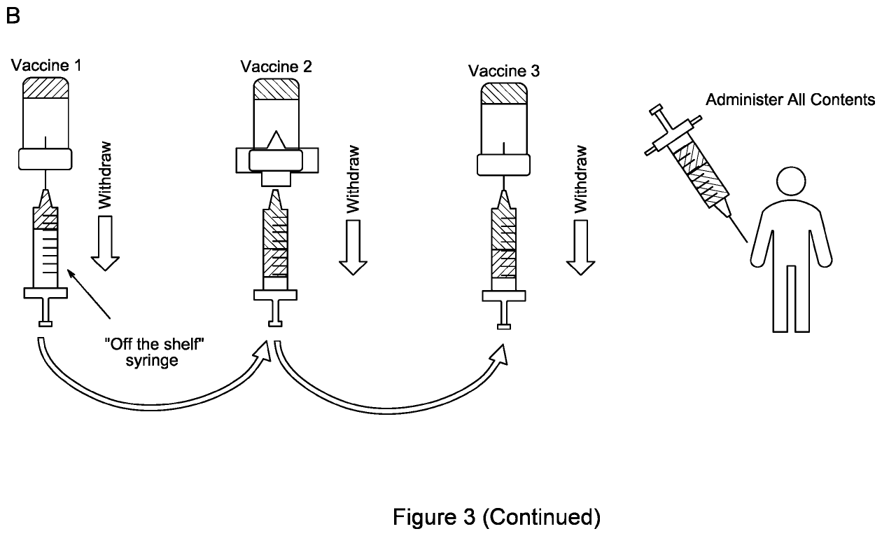

FIG. 3 schematizes the general structure of the antigen-encoding RNAs.

Nucleoside Modified Messenger RNA (modRNA)

The active principle of nucleoside modified RNA (modRNA) drug substance is a single-stranded RNA (e.g., mRNA) that can be translated upon entering a cell. In addition to the sequence encoding a vaccine antigen (i.e. open reading frame), each modRNA contains common structural elements optimized for maximal efficacy of an RNA as the uRNA (5′-cap, 5′-UTR, 3′-UTR, poly(A)-tail). Compared to uRNA, modRNA contains 1-methyl-pseudouridine instead of uridine. The preferred 5′ cap structure is m₂^{7,3′-O}Gppp(m₁^{2′-O})ApG. The preferred 5′-UTR and 3′-UTR comprise the nucleotide sequence of SEQ ID NO: 12 and the nucleotide sequence of SEQ ID NO: 13, respectively. The preferred poly(A)-tail comprises the sequence of SEQ ID NO: 14. An additional purification step is applied for modRNA to reduce dsRNA contaminants generated during the in vitro transcription reaction.

Different embodiment of this platform are as follows:

### BNT162b2; RBP020.1 (SEQ ID NO: 19; SEQ ID NO: 7)

Structure m₂^{7,3′-O}Gppp(m₁^{2′-O})ApG)-hAg-Kozak-S1S2-PP-FI-A30L70
Encoded antigen Viral spike protein (S1S2 protein) of the SARS-CoV-2 (S1S2 full-length protein, sequence variant)

### BNT162b2; RBP020.2 (SEQ ID NO: 20; SEQ ID NO: 7)

Structure m₂^{7,3′-O}Gppp(m₁^{2′-O})ApG)-hAg-Kozak-S1S2-PP-FI-A30L70
Encoded antigen Viral spike protein (S1S2 protein) of the SARS-CoV-2 (S1S2 full-length protein, sequence variant)

### BNT162b1; RBP020.3 (SEQ ID NO: 21; SEQ ID NO: 5)

Structure m₂^{7,3′-O}Gppp(m₁^{2′-O})ApG)-hAg-Kozak-RBD-GS-Fibritin-FI-A30L70
Encoded antigen Viral spike protein (S1S2 protein) of the SARS-CoV-2 (partial sequence, Receptor Binding Domain (RBD) of S1S2 protein fused to fibritin)

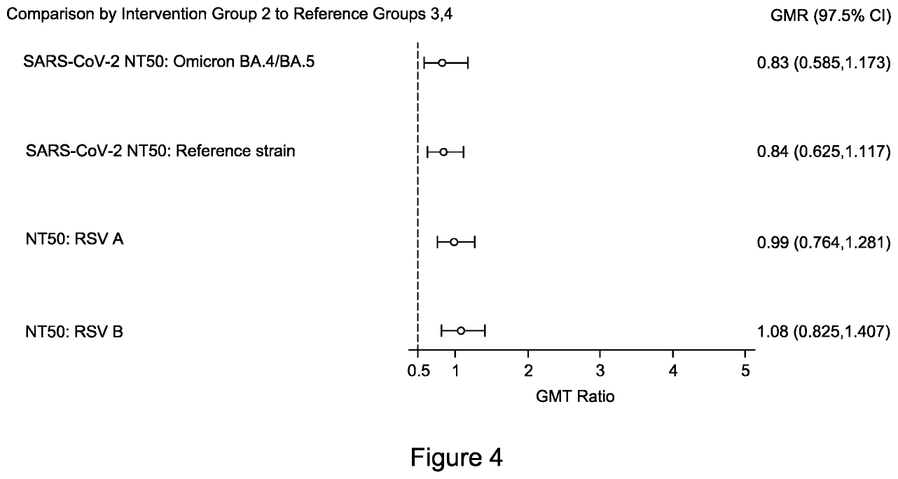

FIG. 4 schematizes the general structure of the antigen-encoding RNAs.

### BNT162b3c (SEQ ID NO: 29; SEQ ID NO: 30)

Structure m₂^{7,3′-O}Gppp(m₁^{2′-O})ApG-hAg-Kozak-RBD-GS-Fibritin-GS-TM-FI-A30L70
Encoded antigen Viral spike protein (S1S2 protein) of the SARS-CoV-2 (partial sequence, Receptor Binding Domain (RBD) of S1S2 protein fused to Fibritin fused to Transmembrane Domain (TM) of S1S2 protein); intrinsic S1S2 protein secretory signal peptide (aa 1-19) at the N-terminus of the antigen sequence

### BNT162b3d (SEQ ID NO: 31; SEQ ID NO: 32)

Structure m₂^{7,3′-O}Gppp(m₁^{2′-O})ApG-hAg-Kozak-RBD-GS-Fibritin-GS-TM-FI-A30L70
Encoded antigen Viral spike protein (S1S2 protein) of the SARS-CoV-2 (partial sequence, Receptor Binding Domain (RBD) of S1S2 protein fused to Fibritin fused to Transmembrane Domain (TM) of S1S2 protein); immunoglobulin secretory signal peptide (aa 1-22) at the N-terminus of the antigen sequence.

### BNT162b2—Beta Variant; RBP020.11 (SEQ ID NO: 57; SEQ ID NO: 55)

Structure m₂^{7,3′-O}Gppp(m₁^{2′-O})ApG)-hAg-Kozak-S1S2-PP-FI-A30L70
Encoded antigen Viral spike protein (S1S2 protein) of the SARS-CoV-2 (S1S2 full-length protein, sequence variant), comprising mutations characteristic of the Beta variant of SARS-CoV-2

### BNT162b2—Alpha Variant; RBP020.14 (SEQ ID NO: 60; SEQ ID NO: 58)

Structure m₂^{7,3′-O}Gppp(m₁^{2′-O})ApG)-hAg-Kozak-S1S2-PP-FI-A30L70
Encoded antigen Viral spike protein (S1S2 protein) of the SARS-CoV-2 (S1S2 full-length protein, sequence variant), comprising mutations characteristic of the Alpha variant of SARS-CoV-2

### BNT162b2—Delta Variant; RBP020.16 (SEQ ID NO: 63; SEQ ID NO: 61)

Structure m₂^{7,3′-O}Gppp(m₁^{2′-O})ApG)-hAg-Kozak-S1S2-PP-FI-A30L70
Encoded antigen Viral spike protein (S1S2 protein) of the SARS-CoV-2 (S1S2 full-length protein, sequence variant), comprising mutations characteristic of the Delta variant of SARS-CoV-2

TABLE 8
Sequences of RBP020.22 (Omicron BA.4/BA.5-specific SARS-COV-2 RNA vaccine)

| SEQ | Brief | |
|---|---|---|
| ID | Descrip- | |
| NO. | tion | Sequence |
| 69 | Amino | MFVFLVLLPLVSSQCVNLITRTQSYTNSFTRGVYYPDKVFRSSVLHSTQDLFLPFFSNVTWFHAISGTNG |
| | acid se- | TKRFDNPVLPFNDGVYFASTEKSNIIRGWIFGTTLDSKTQSLLIVNNATNVVIKVCEFQFCNDPFLDVYY |
| | quence of | HKNNKSWMESEFRVYSSANNCTFEYVSQPFLMDLEGKQGNFKNLREFVFKNIDGYFKIYSKHTPINLGRD |
| | RNA- | LPQGFSALEPLVDLPIGINITRFQTLLALHRSYLTPGDSSSGWTAGAAAYYVGYLQPRTFLLKYNENGTI |
| | encoded | TDAVDCALDPLSETKCTLKSFTVEKGIYQTSNFRVQPTESIVRFPNITNLCPFDEVFNATRFASVYAWNR |
| | SARS- | KRISNCVADYSVLYNFAPFFAFKCYGVSPTKLNDLCFTNVYADSFVIRGNEVRQIAPGQTGNIADYNYKL |
| | COV-2 S | PDDFTGCVIAWNSNKLDSKVGGNYNYRYRLFRKSNLKPFERDISTEIYQAGNKPCNGVAGVNCYFPLQSY |
| | protein | GFRPTYGVGHQPYRVVVLSFELLHAPATVCGPKKSTNLVKNKCVNFNFNGLTGTGVLTESNKKFLPFQQF |
| | from an | GRDIADTTDAVRDPQTLEILDITPCSFGGVSVITPGTNTSNQVAVLYQGVNCTEVPVAIHADQLTPTWRV |
| | Omicron | YSTGSNVFQTRAGCLIGAEYVNNSYECDIPIGAGICASYQTQTKSHRRARSVASQSIIAYTMSLGAENSV |
| | BA.4/BA.5 | AYSNNSIAIPTNFTISVTTEILPVSMTKTSVDCTMYICGDSTECSNLLLQYGSFCTQLKRALTGIAVEQD |
| | variant | KNTQEVFAQVKQIYKTPPIKYFGGFNFSQILPDPSKPSKRSFIEDLLFNKVTLADAGFIKQYGDCLGDIA |
| | (with PRO | ARDLICAQKFNGLTVLPPLLTDEMIAQYTSALLAGTITSGWTFGAGAALQIPFAMQMAYRFNGIGVTQNV |
| | mutations | LYENQKLIANQFNSAIGKIQDSLSSTASALGKLQDVVNHNAQALNTLVKQLSSKFGAISSVLNDILSRLD |
| | at posi- | PPEAEVQIDRLITGRLQSLQTYVTQQLIRAAEIRASANLAATKMSECVLGQSKRVDFCGKGYHLMSFPQS |
| | tions | APHGVVFLHVTYVPAQEKNFTTAPAICHDGKAHFPREGVFVSNGTHWFVTQRNFYEPQIITTDNTFVSGN |
| | corres- | CDVVIGIVNNTVYDPLQPELDSFKEELDKYFKNHTSPDVDLGDISGINASVVNIQKEIDRLNEVAKNLNE |
| | ponding | SLIDLQELGKYEQYIKWPWYIWLGFIAGLIAIVMVTIMLCCMTSCCSCLKGCCSCGSCCKFDEDDSEPVL |
| | to | KGVKLHYT** |
| | positions | |
| | K986P and | |
| | V987P of | |
| | SEQ ID | |
| | NO: 7, | |
| | i.e., at | |
| | residues | |
| | 981 and | |
| | 982 of | |
| | SEQ ID | |
| | NO: 69) | |
| 70 | RNA se | AUGUUCGUGUUCCUGGUGCUGCUGCCUCUGGUGUCCAGCCAGUGUGUGAACCUGAUCACCAGAACACAG |
| | quence | UCAUACACCAACAGCUUUACCAGAGGCGUGUACUACCCCGACAAGGUGUUCAGAUCCAGCGUGCUGCACU |
| | encoding | CUACCCAGGACCUGUUCCUGCCUUUCUUCAGCAACGUGACCUGGUUCCACGCCAUCUCCGGCACCAAUGG |
| | a SARS- | CACCAAGAGAUUCGACAACCCCGUGCUGCCCUUCAACGACGGGGUGUACUUUGCCAGCACCGAGAAGUCC |
| | COV-2 | AACAUCAUCAGAGGCUGGAUCUUCGGCACCACACUGGACAGCAAGACCCAGAGCCUGCUGAUCGUGAACA |
| | S protein | ACGCCACCAACGUGGUCAUCAAAGUGUGCGAGUUCCAGUUCUGCAACGACCCCUUCCUGGACGUCUACUA |
| | from an | CCACAAGAACAACAAGAGCUGGAUGGAAAGCGAGUUCCGGGUGUACAGCAGCGCCAACAACUGCACCUUC |
| | Omicron | GAGUACGUGUCCCAGCCUUUCCUGAUGGACCUGGAAGGCAAGCAGGGCAACUUCAAGAACCUGCGCGAGU |
| | BA.4/BA.5 | UCGUGUUUAAGAACAUCGACGGCUACUUCAAGAUCUACAGCAAGCACACCCCUAUCAACCUCGGCCGGGA |
| | variant | UCUGCCUCAGGGCUUCUCUGCUCUGGAACCCCUGGUGGAUCUGCCCAUCGGCAUCAACAUCACCCGGUUU |
| | | CAGACACUGCUGGCCCUGCACAGAAGCUACCUGACACCUGGCGAUAGCAGCAGCGGAUGGACAGCUGGUG |
| | | CCGCCGCUUACUAUGUGGGCUACCUGCAGCCUAGAACCUUCCUGCUGAAGUACAACGAGAACGGCACCAU |
| | | CACCGACGCCGUGGAUUGUGCUCUGGAUCCUCUGAGCGAGACAAAGUGCACCCUGAAGUCCUUCACCGUG |
| | | GAAAAGGGCAUCUACCAGACCAGCAACUUCCGGGUGCAGCCCACCGAAUCCAUCGUGCGGUUCCCCAAUA |
| | | UCACCAAUCUGUGCCCCUUCGACGAGGUGUUCAAUGCCACCAGAUUCGCCUCUGUGUACGCCUGGAACCG |
| | | GAAGCGGAUCAGCAAUUGCGUGGCCGACUACUCCGUGCUGUACAACUUCGCCCCCUUCUUCGCAUUCAAG |
| | | UGCUACGGCGUGUCCCCUACCAAGCUGAACGACCUGUGCUUCACAAACGUGUACGCCGACAGCUUCGUGA |
| | | UCCGGGGAAACGAAGUGCGGCAGAUUGCCCCUGGACAGACAGGCAACAUCGCCGACUACAACUACAAGCU |
| | | GCCCGACGACUUCACCGGCUGUGUGAUUGCCUGGAACAGCAACAAGCUGGACUCCAAAGUCGGCGGCAAC |
| | | UACAAUUACAGGUACCGGCUGUUCCGGAAGUCCAAUCUGAAGCCCUUCGAGCGGGACAUCUCCACCGAGA |
| | | UCUAUCAGGCCGGCAACAAGCCUUGUAACGGCGUGGCAGGCGUGAACUGCUACUUCCCACUGCAGUCCUA |
| | | CGGCUUUAGGCCCACAUACGGCGUGGGCCACCAGCCCUACAGAGUGGUGGUGCUGAGCUUCGAACUGCU |
| | | GCAUGCCCCUGCCACAGUGUGCGGCCCUAAGAAAAGCACCAAUCUCGUGAAGAACAAAUGCGUGAACUUC |
| | | AACUUCAACGGCCUGACCGGCACCGGCGUGCUGACAGAGAGCAACAAGAAGUUCCUGCCAUUCCAGCAGU |
| | | UUGGCCGGGAUAUCGCCGAUACCACAGACGCCGUUAGAGAUCCCCAGACACUGGAAAUCCUGGACAUCAC |
| | | CCCUUGCAGCUUCGGCGGAGUGUCUGUGAUCACCCCUGGCACCAACACCAGCAAUCAGGUGGCAGUGCUG |
| | | UACCAGGGCGUGAACUGUACCGAAGUGCCCGUGGCCAUUCACGCCGAUCAGCUGACACCUACAUGGGGG |
| | | UGUACUCCACCGGCAGCAAUGUGUUUCAGACCAGAGCCGGCUGUCUGAUCGGAGCCGAGUACGUGAACAA |
| | | UAGCUACGAGUGCGACAUCCCCAUCGGCGCUGGAAUCUGCGCCAGCUACCAGACACAGACAAAGAGCCACC |
| | | GGAGAGCCAGAAGCGUGGCCAGCCAGAGCAUCAUUGCCUACACAAUGUCUCUGGGCGCCGAGAACAGCGU |
| | | GGCCUACUCCAACAACUCUAUCGCUAUCCCCACCAACUUCACCAUCAGCGUGACCACAGAGAUCCUGCCUG |
| | | UGUCCAUGACCAAGACCAGCGUGGACUGCACCAUGUACAUCUGCGGCGAUUCCACCGAGUGCUCCAACCU |
| | | GCUGCUGCAGUACGGCAGCUUCUGCACCCAGCUGAAAAGAGCCCUGACAGGGAUCGCCGUGGAACAGGAC |
| | | AAGAACACCCAAGAGGUGUUCGCCCAAGUGAAGCAGAUCUACAAGACCCCUCCUAUCAAGUACUUCGGCG |
| | | GCUUCAAUUUCAGCCAGAUUCUGCCCGAUCCUAGCAAGCCCAGCAAGCGGAGCUUCAUCGAGGACCUGCU |
| | | GUUCAACAAAGUGACACUGGCCGACGCCGGCUUCAUCAAGCAGUAUGGCGAUUGUCUGGGCGACAUUGCC |
| | | GCCAGGGAUCUGAUUUGCGCCCAGAAGUUUAACGGACUGACAGUGCUGCCUCCUCUGCUGACCGAUGAGA |
| | | UGAUCGCCCAGUACACAUCUGCCCUGCUGGCCGGCACAAUCACAAGCGGCUGGACAUUUGGAGCAGGCGC |
| | | CGCUCUGCAGAUCCCCUUUGCUAUGCAGAUGGCCUACCGGUUCAACGGCAUCGGAGUGACCCAGAAUGUG |
| | | CUGUACGAGAACCAGAAGCUGAUCGCCAACCAGUUCAACAGCGCCAUCGGCAAGAUCCAGGACAGCCUGA |
| | | GCAGCACAGCAAGCGCCCUGGGAAAGCUGCAGGACGUGGUCAACCACAAUGCCCAGGCACUGAACACCCU |
| | | GGUCAAGCAGCUGUCCUCCAAGUUCGGCGCCAUCAGCUCUGUGCUGAACGAUAUCCUGAGCAGACUGGAC |
| | | CCUCCUGAGGCCGAGGUGCAGAUCGACAGACUGAUCACAGGCAGACUGCAGAGCCUCCAGACAUACGUGA |
| | | CCCAGCAGCUGAUCAGAGCCGCCGAGAUUAGAGCCUCUGCCAAUCUGGCCGCCACCAAGAUGUCUGAGUG |
| | | UGUGCUGGGCCAGAGCAAGAGAGUGGACUUUUGCGGCAAGGGCUACCACCUGAUGAGCUUCCCUCAGUC |
| | | UGCCCCUCACGGCGUGGUGUUUCUGCACGUGACAUAUGUGCCCGCUCAAGAGAAGAAUUUCACCACCGCU |
| | | CCAGCCAUCUGCCACGACGGCAAAGCCCACUUUCCUAGAGAAGGCGUGUUCGUGUCCAACGGCACCCAUU |
| | | GGUUCGUGACACAGCGGAACUUCUACGAGCCCCAGAUCAUCACCACCGACAACACCUUCGUGUCUGGCAA |
| | | CUGCGACGUCGUGAUCGGCAUUGUGAACAAUACCGUGUACGACCCUCUGCAGCCCGAGCUGGACAGCUUC |
| | | AAAGAGGAACUGGACAAGUACUUUAAGAACCACACAAGCCCCGACGUGGACCUGGGCGAUAUCAGCGGAA |
| | | UCAAUGCCAGCGUCGUGAACAUCCAGAAAGAGAUCGACCGGCUGAACGAGGUGGCCAAGAAUCUGAACGA |
| | | GAGCCUGAUCGACCUGCAAGAACUGGGGAAGUACGAGCAGUACAUCAAGUGGCCCUGGUACAUCUGGCUG |
| | | GGCUUUAUCGCCGGACUGAUUGCCAUCGUGAUGGUCACAAUCAUGCUGUGUUGCAUGACCAGCUGCUGU |
| | | AGCUGCCUGAAGGGCUGUUGUAGCUGUGGCAGCUGCUGCAAGUUCGACGAGGACGAUUCUGAGCCCGUG |
| | | CUGAAGGGCGUGAAACUGCACUACACAUGAUGA |
| 71 | DNA se- | ATGTTCGTGTTCCTGGTGCTGCTGCCTCTGGTGTCCAGCCAGTGTGTGAACCTGATCACCAGAACACAGTCA |
| | quence | TACACCAACAGCTTTACCAGAGGCGTGTACTACCCCGACAAGGTGTTCAGATCCAGCGTGCTGCACTCTACC |
| | encoding | CAGGACCTGTTCCTGCCTTTCTTCAGCAACGTGACCTGGTTCCACGCCATCTCCGGCACCAATGGCACCAAG |
| | a SARS- | AGATTCGACAACCCCGTGCTGCCCTTCAACGACGGGGTGTACTTTGCCAGCACCGAGAAGTCCAACATCATC |
| | COV-2 S | AGAGGCTGGATCTTCGGCACCACACTGGACAGCAAGACCCAGAGCCTGCTGATCGTGAACAACGCCACCAAC |
| | protein | GTGGTCATCAAAGTGTGCGAGTTCCAGTTCTGCAACGACCCCTTCCTGGACGTCTACTACCACAAGAACAAC |
| | from an | AAGAGCTGGATGGAAAGCGAGTTCCGGGTGTACAGCAGCGCCAACAACTGCACCTTCGAGTACGTGTCCCA |
| | Omicron | GCCTTTCCTGATGGACCTGGAAGGCAAGCAGGGCAACTTCAAGAACCTGCGCGAGTTCGTGTTTAAGAACAT |
| | BA.4/BA.5 | CGACGGCTACTTCAAGATCTACAGCAAGCACACCCCTATCAACCTCGGCCGGGATCTGCCTCAGGGCTTCTC |
| | variant | TGCTCTGGAACCCCTGGTGGATCTGCCCATCGGCATCAACATCACCCGGTTTCAGACACTGCTGGCCCTGCA |
| | | CAGAAGCTACCTGACACCTGGCGATAGCAGCAGCGGATGGACAGCTGGTGCCGCCGCTTACTATGTGGGCT |
| | | ACCTGCAGCCTAGAACCTTCCTGCTGAAGTACAACGAGAACGGCACCATCACCGACGCCGTGGATTGTGCTC |
| | | TGGATCCTCTGAGCGAGACAAAGTGCACCCTGAAGTCCTTCACCGTGGAAAAGGGCATCTACCAGACCAGCA |
| | | ACTTCCGGGTGCAGCCCACCGAATCCATCGTGCGGTTCCCCAATATCACCAATCTGTGCCCCTTCGACGAGG |
| | | TGTTCAATGCCACCAGATTCGCCTCTGTGTACGCCTGGAACCGGAAGCGGATCAGCAATTGCGTGGCCGACT |
| | | ACTCCGTGCTGTACAACTTCGCCCCCTTCTTCGCATTCAAGTGCTACGGCGTGTCCCCTACCAAGCTGAACG |
| | | ACCTGTGCTTCACAAACGTGTACGCCGACAGCTTCGTGATCCGGGGAAACGAAGTGCGGCAGATTGCCCCT |
| | | GGACAGACAGGCAACATCGCCGACTACAACTACAAGCTGCCCGACGACTTCACCGGCTGTGTGATTGCCTGG |
| | | AACAGCAACAAGCTGGACTCCAAAGTCGGCGGCAACTACAATTACAGGTACCGGCTGTTCCGGAAGTCCAAT |
| | | CTGAAGCCCTTCGAGCGGGACATCTCCACCGAGATCTATCAGGCCGGCAACAAGCCTTGTAACGGCGTGGC |
| | | AGGCGTGAACTGCTACTTCCCACTGCAGTCCTACGGCTTTAGGCCCACATACGGCGTGGGCCACCAGCCCTA |
| | | CAGAGTGGTGGTGCTGAGCTTCGAACTGCTGCATGCCCCTGCCACAGTGTGCGGCCCTAAGAAAAGCACCA |
| | | ATCTCGTGAAGAACAAATGCGTGAACTTCAACTTCAACGGCCTGACCGGCACCGGCGTGCTGACAGAGAGCA |
| | | ACAAGAAGTTCCTGCCATTCCAGCAGTTTGGCCGGGATATCGCCGATACCACAGACGCCGTTAGAGATCCCC |
| | | AGACACTGGAAATCCTGGACATCACCCCTTGCAGCTTCGGCGGAGTGTCTGTGATCACCCCTGGCACCAACA |
| | | CCAGCAATCAGGTGGCAGTGCTGTACCAGGGCGTGAACTGTACCGAAGTGCCCGTGGCCATTCACGCCGAT |
| | | CAGCTGACACCTACATGGCGGGTGTACTCCACCGGCAGCAATGTGTTTCAGACCAGAGCCGGCTGTCTGAT |
| | | CGGAGCCGAGTACGTGAACAATAGCTACGAGTGCGACATCCCCATCGGCGCTGGAATCTGCGCCAGCTACC |
| | | AGACACAGACAAAGAGCCACCGGAGAGCCAGAAGCGTGGCCAGCCAGAGCATCATTGCCTACACAATGTCTC |
| | | TGGGCGCCGAGAACAGCGTGGCCTACTCCAACAACTCTATCGCTATCCCCACCAACTTCACCATCAGCGTGA |
| | | CCACAGAGATCCTGCCTGTGTCCATGACCAAGACCAGCGTGGACTGCACCATGTACATCTGCGGCGATTCCA |
| | | CCGAGTGCTCCAACCTGCTGCTGCAGTACGGCAGCTTCTGCACCCAGCTGAAAAGAGCCCTGACAGGGATC |
| | | GCCGTGGAACAGGACAAGAACACCCAAGAGGTGTTCGCCCAAGTGAAGCAGATCTACAAGACCCCTCCTATC |
| | | AAGTACTTCGGCGGCTTCAATTTCAGCCAGATTCTGCCCGATCCTAGCAAGCCCAGCAAGCGGAGCTTCATC |
| | | GAGGACCTGCTGTTCAACAAAGTGACACTGGCCGACGCCGGCTTCATCAAGCAGTATGGCGATTGTCTGGG |
| | | CGACATTGCCGCCAGGGATCTGATTTGCGCCCAGAAGTTTAACGGACTGACAGTGCTGCCTCCTCTGCTGAC |
| | | CGATGAGATGATCGCCCAGTACACATCTGCCCTGCTGGCCGGCACAATCACAAGCGGCTGGACATTTGGAG |
| | | CAGGCGCCGCTCTGCAGATCCCCTTTGCTATGCAGATGGCCTACCGGTTCAACGGCATCGGAGTGACCCAG |
| | | AATGTGCTGTACGAGAACCAGAAGCTGATCGCCAACCAGTTCAACAGCGCCATCGGCAAGATCCAGGACAGC |
| | | CTGAGCAGCACAGCAAGCGCCCTGGGAAAGCTGCAGGACGTGGTCAACCACAATGCCCAGGCACTGAACAC |
| | | CCTGGTCAAGCAGCTGTCCTCCAAGTTCGGCGCCATCAGCTCTGTGCTGAACGATATCCTGAGCAGACTGGA |
| | | CCCTCCTGAGGCCGAGGTGCAGATCGACAGACTGATCACAGGCAGACTGCAGAGCCTCCAGACATACGTGA |
| | | CCCAGCAGCTGATCAGAGCCGCCGAGATTAGAGCCTCTGCCAATCTGGCCGCCACCAAGATGTCTGAGTGT |
| | | GTGCTGGGCCAGAGCAAGAGAGTGGACTTTTGCGGCAAGGGCTACCACCTGATGAGCTTCCCTCAGTCTGC |
| | | CCCTCACGGCGTGGTGTTTCTGCACGTGACATATGTGCCCGCTCAAGAGAAGAATTTCACCACCGCTCCAGC |
| | | CATCTGCCACGACGGCAAAGCCCACTTTCCTAGAGAAGGCGTGTTCGTGTCCAACGGCACCCATTGGTTCGT |
| | | GACACAGCGGAACTTCTACGAGCCCCAGATCATCACCACCGACAACACCTTCGTGTCTGGCAACTGCGACGT |
| | | CGTGATCGGCATTGTGAACAATACCGTGTACGACCCTCTGCAGCCCGAGCTGGACAGCTTCAAAGAGGAACT |
| | | GGACAAGTACTTTAAGAACCACACAAGCCCCGACGTGGACCTGGGCGATATCAGCGGAATCAATGCCAGCG |
| | | TCGTGAACATCCAGAAAGAGATCGACCGGCTGAACGAGGTGGCCAAGAATCTGAACGAGAGCCTGATCGAC |
| | | CTGCAAGAACTGGGGAAGTACGAGCAGTACATCAAGTGGCCCTGGTACATCTGGCTGGGCTTTATCGCCGG |
| | | ACTGATTGCCATCGTGATGGTCACAATCATGCTGTGTTGCATGACCAGCTGCTGTAGCTGCCTGAAGGGCT |
| | | GTTGTAGCTGTGGCAGCTGCTGCAAGTTCGACGAGGACGATTCTGAGCCCGTGCTGAAGGGCGTGAAACTG |
| | | CACTACACATGATGA |
| 72 | Full | AGAAUAAACUAGUAUUCUUCUGGUCCCCACAGACUCAGAGAGAACCCGCCACCAUGUUCGUGUUCCUGGU |
| | length | GCUGCUGCCUCUGGUGUCCAGCCAGUGUGUGAACCUGAUCACCAGAACACAGUCAUACACCAACAGCUUU |
| | RNA con- | ACCAGAGGCGUGUACUACCCCGACAAGGUGUUCAGAUCCAGCGUGCUGCACUCUACCCAGGACCUGUUCC |
| | struct | UGCCUUUCUUCAGCAACGUGACCUGGUUCCACGCCAUCUCCGGCACCAAUGGCACCAAGAGAUUCGACAA |
| | sequence | CCCCGUGCUGCCCUUCAACGACGGGGUGUACUUUGCCAGCACCGAGAAGUCCAACAUCAUCAGAGGCUGG |
| | of | AUCUUCGGCACCACACUGGACAGCAAGACCCAGAGCCUGCUGAUCGUGAACAACGCCACCAACGUGGUCAU |
| | RBP020.22 | CAAAGUGUGCGAGUUCCAGUUCUGCAACGACCCCUUCCUGGACGUCUACUACCACAAGAACAACAAGAGC |
| | | UGGAUGGAAAGCGAGUUCCGGGUGUACAGCAGCGCCAACAACUGCACCUUCGAGUACGUGUCCCAGCCUU |
| | | UCCUGAUGGACCUGGAAGGCAAGCAGGGCAACUUCAAGAACCUGCGCGAGUUCGUGUUUAAGAACAUCGA |
| | | CGGCUACUUCAAGAUCUACAGCAAGCACACCCCUAUCAACCUCGGCCGGGAUCUGCCUCAGGGCUUCUCU |
| | | GCUCUGGAACCCCUGGUGGAUCUGCCCAUCGGCAUCAACAUCACCCGGUUUCAGACACUGCUGGCCCUGC |
| | | ACAGAAGCUACCUGACACCUGGCGAUAGCAGCAGCGGAUGGACAGCUGGUGCCGCCGCUUACUAUGUGGG |
| | | CUACCUGCAGCCUAGAACCUUCCUGCUGAAGUACAACGAGAACGGCACCAUCACCGACGCCGUGGAUUGU |
| | | GCUCUGGAUCCUCUGAGCGAGACAAAGUGCACCCUGAAGUCCUUCACCGUGGAAAAGGGCAUCUACCAGA |
| | | CCAGCAACUUCCGGGUGCAGCCCACCGAAUCCAUCGUGCGGUUCCCCAAUAUCACCAAUCUGUGCCCCUU |
| | | CGACGAGGUGUUCAAUGCCACCAGAUUCGCCUCUGUGUACGCCUGGAACCGGAAGCGGAUCAGCAAUUGC |
| | | GUGGCCGACUACUCCGUGCUGUACAACUUCGCCCCCUUCUUCGCAUUCAAGUGCUACGGCGUGUCCCCUA |
| | | CCAAGCUGAACGACCUGUGCUUCACAAACGUGUACGCCGACAGCUUCGUGAUCCGGGGAAACGAAGUGCG |
| | | GCAGAUUGCCCCUGGACAGACAGGCAACAUCGCCGACUACAACUACAAGCUGCCCGACGACUUCACCGGCU |
| | | GUGUGAUUGCCUGGAACAGCAACAAGCUGGACUCCAAAGUCGGCGGCAACUACAAUUACAGGUACCGGCU |
| | | GUUCCGGAAGUCCAAUCUGAAGCCCUUCGAGCGGGACAUCUCCACCGAGAUCUAUCAGGCCGGCAACAAG |
| | | CCUUGUAACGGCGUGGCAGGCGUGAACUGCUACUUCCCACUGCAGUCCUACGGCUUUAGGCCCACAUACG |
| | | GCGUGGGCCACCAGCCCUACAGAGUGGUGGUGCUGAGCUUCGAACUGCUGCAUGCCCCUGCCACAGUGU |
| | | GCGGCCCUAAGAAAAGCACCAAUCUCGUGAAGAACAAAUGCGUGAACUUCAACUUCAACGGCCUGACCGGC |
| | | ACCGGCGUGCUGACAGAGAGCAACAAGAAGUUCCUGCCAUUCCAGCAGUUUGGCCGGGAUAUCGCCGAUA |
| | | CCACAGACGCCGUUAGAGAUCCCCAGACACUGGAAAUCCUGGACAUCACCCCUUGCAGCUUCGGCGGAGU |
| | | GUCUGUGAUCACCCCUGGCACCAACACCAGCAAUCAGGUGGCAGUGCUGUACCAGGGCGUGAACUGUACC |
| | | GAAGUGCCCGUGGCCAUUCACGCCGAUCAGCUGACACCUACAUGGCGGGUGUACUCCACCGGCAGCAAUG |
| | | UGUUUCAGACCAGAGCCGGCUGUCUGAUCGGAGCCGAGUACGUGAACAAUAGCUACGAGUGCGACAUCCC |
| | | CAUCGGCGCUGGAAUCUGCGCCAGCUACCAGACACAGACAAAGAGCCACCGGAGAGCCAGAAGCGUGGCC |
| | | AGCCAGAGCAUCAUUGCCUACACAAUGUCUCUGGGCGCCGAGAACAGCGUGGCCUACUCCAACAACUCUA |
| | | UCGCUAUCCCCACCAACUUCACCAUCAGCGUGACCACAGAGAUCCUGCCUGUGUCCAUGACCAAGACCAGC |
| | | GUGGACUGCACCAUGUACAUCUGCGGCGAUUCCACCGAGUGCUCCAACCUGCUGCUGCAGUACGGCAGCU |
| | | UCUGCACCCAGCUGAAAAGAGCCCUGACAGGGAUCGCCGUGGAACAGGACAAGAACACCCAAGAGGUGUU |
| | | CGCCCAAGUGAAGCAGAUCUACAAGACCCCUCCUAUCAAGUACUUCGGCGGCUUCAAUUUCAGCCAGAUU |
| | | CUGCCCGAUCCUAGCAAGCCCAGCAAGCGGAGCUUCAUCGAGGACCUGCUGUUCAACAAAGUGACACUGG |
| | | CCGACGCCGGCUUCAUCAAGCAGUAUGGCGAUUGUCUGGGCGACAUUGCCGCCAGGGAUCUGAUUUGCG |
| | | CCCAGAAGUUUAACGGACUGACAGUGCUGCCUCCUCUGCUGACCGAUGAGAUGAUCGCCCAGUACACAUC |
| | | UGCCCUGCUGGCCGGCACAAUCACAAGCGGCUGGACAUUUGGAGCAGGCGCCGCUCUGCAGAUCCCCUUU |
| | | GCUAUGCAGAUGGCCUACCGGUUCAACGGCAUCGGAGUGACCCAGAAUGUGCUGUACGAGAACCAGAAGC |
| | | UGAUCGCCAACCAGUUCAACAGCGCCAUCGGCAAGAUCCAGGACAGCCUGAGCAGCACAGCAAGCGCCCUG |
| | | GGAAAGCUGCAGGACGUGGUCAACCACAAUGCCCAGGCACUGAACACCCUGGUCAAGCAGCUGUCCUCCA |
| | | AGUUCGGCGCCAUCAGCUCUGUGCUGAACGAUAUCCUGAGCAGACUGGACCCUCCUGAGGCCGAGGUGCA |
| | | GAUCGACAGACUGAUCACAGGCAGACUGCAGAGCCUCCAGACAUACGUGACCCAGCAGCUGAUCAGAGCC |
| | | GCCGAGAUUAGAGCCUCUGCCAAUCUGGCCGCCACCAAGAUGUCUGAGUGUGUGCUGGGCCAGAGCAAGA |
| | | GAGUGGACUUUUGCGGCAAGGGCUACCACCUGAUGAGCUUCCCUCAGUCUGCCCCUCACGGCGUGGUGU |
| | | UUCUGCACGUGACAUAUGUGCCCGCUCAAGAGAAGAAUUUCACCACCGCUCCAGCCAUCUGCCACGACGG |
| | | CAAAGCCCACUUUCCUAGAGAAGGCGUGUUCGUGUCCAACGGCACCCAUUGGUUCGUGACACAGCGGAAC |
| | | UUCUACGAGCCCCAGAUCAUCACCACCGACAACACCUUCGUGUCUGGCAACUGCGACGUCGUGAUCGGCA |
| | | UUGUGAACAAUACCGUGUACGACCCUCUGCAGCCCGAGCUGGACAGCUUCAAAGAGGAACUGGACAAGUA |
| | | CUUUAAGAACCACACAAGCCCCGACGUGGACCUGGGCGAUAUCAGCGGAAUCAAUGCCAGCGUCGUGAAC |
| | | AUCCAGAAAGAGAUCGACCGGCUGAACGAGGUGGCCAAGAAUCUGAACGAGAGCCUGAUCGACCUGCAAG |
| | | AACUGGGGAAGUACGAGCAGUACAUCAAGUGGCCCUGGUACAUCUGGCUGGGCUUUAUCGCCGGACUGA |
| | | UUGCCAUCGUGAUGGUCACAAUCAUGCUGUGUUGCAUGACCAGCUGCUGUAGCUGCCUGAAGGGCUGUU |
| | | GUAGCUGUGGCAGCUGCUGCAAGUUCGACGAGGACGAUUCUGAGCCCGUGCUGAAGGGCGUGAAACUGC |
| | | ACUACACAUGAUGACUCGAGCUGGUACUGCAUGCACGCAAUGCUAGCUGCCCCUUUCCCGUCCUGGGUAC |
| | | CCCGAGUCUCCCCCGACCUCGGGUCCCAGGUAUGCUCCCACCUCCACCUGCCCCACUCACCACCUCUGCUA |
| | | GUUCCAGACACCUCCCAAGCACGCAGCAAUGCAGCUCAAAACGCUUAGCCUAGCCACACCCCCACGGGAAA |
| | | CAGCAGUGAUUAACCUUUAGCAAUAAACGAAAGUUUAACUAAGCUAUACUAACCCCAGGGUUGGUCAAUU |
| | | UCGUGCCAGCCACACCCUGGAGCUAGCAAAAAAAAAAAAAAAAAAAAAAAAAAAAAAGCAUAUGACUAAAAA |
| | | AAAAAAAAAAAAAAAAAAAAAAAAAAAAAAAAAAAAAAAAAAAAAAAAAAAAAAAAAAAAAAAAA |
| 73 | Full | AGAATAAACTAGTATTCTTCTGGTCCCCACAGACTCAGAGAGAACCCGCCACCATGTTCGTGTTCCTGGTGC |
| | length | TGCTGCCTCTGGTGTCCAGCCAGTGTGTGAACCTGATCACCAGAACACAGTCATACACCAACAGCTTTACCA |
| | DNA con- | GAGGCGTGTACTACCCCGACAAGGTGTTCAGATCCAGCGTGCTGCACTCTACCCAGGACCTGTTCCTGCCTT |
| | struct | TCTTCAGCAACGTGACCTGGTTCCACGCCATCTCCGGCACCAATGGCACCAAGAGATTCGACAACCCCGTGC |
| | sequence | TGCCCTTCAACGACGGGGTGTACTTTGCCAGCACCGAGAAGTCCAACATCATCAGAGGCTGGATCTTCGGCA |
| | of | CCACACTGGACAGCAAGACCCAGAGCCTGCTGATCGTGAACAACGCCACCAACGTGGTCATCAAAGTGTGCG |
| | RBP020.22 | AGTTCCAGTTCTGCAACGACCCCTTCCTGGACGTCTACTACCACAAGAACAACAAGAGCTGGATGGAAAGCG |
| | | AGTTCCGGGTGTACAGCAGCGCCAACAACTGCACCTTCGAGTACGTGTCCCAGCCTTTCCTGATGGACCTGG |
| | | AAGGCAAGCAGGGCAACTTCAAGAACCTGCGCGAGTTCGTGTTTAAGAACATCGACGGCTACTTCAAGATCT |
| | | ACAGCAAGCACACCCCTATCAACCTCGGCCGGGATCTGCCTCAGGGCTTCTCTGCTCTGGAACCCCTGGTGG |
| | | ATCTGCCCATCGGCATCAACATCACCCGGTTTCAGACACTGCTGGCCCTGCACAGAAGCTACCTGACACCTG |
| | | GCGATAGCAGCAGCGGATGGACAGCTGGTGCCGCCGCTTACTATGTGGGCTACCTGCAGCCTAGAACCTTC |
| | | CTGCTGAAGTACAACGAGAACGGCACCATCACCGACGCCGTGGATTGTGCTCTGGATCCTCTGAGCGAGAC |
| | | AAAGTGCACCCTGAAGTCCTTCACCGTGGAAAAGGGCATCTACCAGACCAGCAACTTCCGGGTGCAGCCCAC |
| | | CGAATCCATCGTGCGGTTCCCCAATATCACCAATCTGTGCCCCTTCGACGAGGTGTTCAATGCCACCAGATT |
| | | CGCCTCTGTGTACGCCTGGAACCGGAAGCGGATCAGCAATTGCGTGGCCGACTACTCCGTGCTGTACAACT |
| | | TCGCCCCCTTCTTCGCATTCAAGTGCTACGGCGTGTCCCCTACCAAGCTGAACGACCTGTGCTTCACAAACG |
| | | TGTACGCCGACAGCTTCGTGATCCGGGGAAACGAAGTGCGGCAGATTGCCCCTGGACAGACAGGCAACATC |
| | | GCCGACTACAACTACAAGCTGCCCGACGACTTCACCGGCTGTGTGATTGCCTGGAACAGCAACAAGCTGGAC |
| | | TCCAAAGTCGGCGGCAACTACAATTACAGGTACCGGCTGTTCCGGAAGTCCAATCTGAAGCCCTTCGAGCGG |
| | | GACATCTCCACCGAGATCTATCAGGCCGGCAACAAGCCTTGTAACGGCGTGGCAGGCGTGAACTGCTACTTC |
| | | CCACTGCAGTCCTACGGCTTTAGGCCCACATACGGCGTGGGCCACCAGCCCTACAGAGTGGTGGTGCTGAG |
| | | CTTCGAACTGCTGCATGCCCCTGCCACAGTGTGCGGCCCTAAGAAAAGCACCAATCTCGTGAAGAACAAATG |
| | | CGTGAACTTCAACTTCAACGGCCTGACCGGCACCGGCGTGCTGACAGAGAGCAACAAGAAGTTCCTGCCATT |
| | | CCAGCAGTTTGGCCGGGATATCGCCGATACCACAGACGCCGTTAGAGATCCCCAGACACTGGAAATCCTGGA |
| | | CATCACCCCTTGCAGCTTCGGCGGAGTGTCTGTGATCACCCCTGGCACCAACACCAGCAATCAGGTGGCAGT |
| | | GCTGTACCAGGGCGTGAACTGTACCGAAGTGCCCGTGGCCATTCACGCCGATCAGCTGACACCTACATGGC |
| | | GGGTGTACTCCACCGGCAGCAATGTGTTTCAGACCAGAGCCGGCTGTCTGATCGGAGCCGAGTACGTGAAC |
| | | AATAGCTACGAGTGCGACATCCCCATCGGCGCTGGAATCTGCGCCAGCTACCAGACACAGACAAAGAGCCAC |
| | | CGGAGAGCCAGAAGCGTGGCCAGCCAGAGCATCATTGCCTACACAATGTCTCTGGGCGCCGAGAACAGCGT |
| | | GGCCTACTCCAACAACTCTATCGCTATCCCCACCAACTTCACCATCAGCGTGACCACAGAGATCCTGCCTGT |
| | | GTCCATGACCAAGACCAGCGTGGACTGCACCATGTACATCTGCGGCGATTCCACCGAGTGCTCCAACCTGCT |
| | | GCTGCAGTACGGCAGCTTCTGCACCCAGCTGAAAAGAGCCCTGACAGGGATCGCCGTGGAACAGGACAAGA |
| | | ACACCCAAGAGGTGTTCGCCCAAGTGAAGCAGATCTACAAGACCCCTCCTATCAAGTACTTCGGCGGCTTCA |
| | | ATTTCAGCCAGATTCTGCCCGATCCTAGCAAGCCCAGCAAGCGGAGCTTCATCGAGGACCTGCTGTTCAACA |
| | | AAGTGACACTGGCCGACGCCGGCTTCATCAAGCAGTATGGCGATTGTCTGGGCGACATTGCCGCCAGGGAT |
| | | CTGATTTGCGCCCAGAAGTTTAACGGACTGACAGTGCTGCCTCCTCTGCTGACCGATGAGATGATCGCCCAG |
| | | TACACATCTGCCCTGCTGGCCGGCACAATCACAAGCGGCTGGACATTTGGAGCAGGCGCCGCTCTGCAGAT |
| | | CCCCTTTGCTATGCAGATGGCCTACCGGTTCAACGGCATCGGAGTGACCCAGAATGTGCTGTACGAGAACCA |
| | | GAAGCTGATCGCCAACCAGTTCAACAGCGCCATCGGCAAGATCCAGGACAGCCTGAGCAGCACAGCAAGCG |
| | | CCCTGGGAAAGCTGCAGGACGTGGTCAACCACAATGCCCAGGCACTGAACACCCTGGTCAAGCAGCTGTCC |
| | | TCCAAGTTCGGCGCCATCAGCTCTGTGCTGAACGATATCCTGAGCAGACTGGACCCTCCTGAGGCCGAGGT |
| | | GCAGATCGACAGACTGATCACAGGCAGACTGCAGAGCCTCCAGACATACGTGACCCAGCAGCTGATCAGAG |
| | | CCGCCGAGATTAGAGCCTCTGCCAATCTGGCCGCCACCAAGATGTCTGAGTGTGTGCTGGGCCAGAGCAAG |
| | | AGAGTGGACTTTTGCGGCAAGGGCTACCACCTGATGAGCTTCCCTCAGTCTGCCCCTCACGGCGTGGTGTT |
| | | TCTGCACGTGACATATGTGCCCGCTCAAGAGAAGAATTTCACCACCGCTCCAGCCATCTGCCACGACGGCAA |
| | | AGCCCACTTTCCTAGAGAAGGCGTGTTCGTGTCCAACGGCACCCATTGGTTCGTGACACAGCGGAACTTCTA |
| | | CGAGCCCCAGATCATCACCACCGACAACACCTTCGTGTCTGGCAACTGCGACGTCGTGATCGGCATTGTGAA |
| | | CAATACCGTGTACGACCCTCTGCAGCCCGAGCTGGACAGCTTCAAAGAGGAACTGGACAAGTACTTTAAGAA |
| | | CCACACAAGCCCCGACGTGGACCTGGGCGATATCAGCGGAATCAATGCCAGCGTCGTGAACATCCAGAAAG |
| | | AGATCGACCGGCTGAACGAGGTGGCCAAGAATCTGAACGAGAGCCTGATCGACCTGCAAGAACTGGGGAAG |
| | | TACGAGCAGTACATCAAGTGGCCCTGGTACATCTGGCTGGGCTTTATCGCCGGACTGATTGCCATCGTGAT |
| | | GGTCACAATCATGCTGTGTTGCATGACCAGCTGCTGTAGCTGCCTGAAGGGCTGTTGTAGCTGTGGCAGCT |
| | | GCTGCAAGTTCGACGAGGACGATTCTGAGCCCGTGCTGAAGGGCGTGAAACTGCACTACACATGATGACTC |
| | | GAGCTGGTACTGCATGCACGCAATGCTAGCTGCCCCTTTCCCGTCCTGGGTACCCCGAGTCTCCCCCGACCT |
| | | CGGGTCCCAGGTATGCTCCCACCTCCACCTGCCCCACTCACCACCTCTGCTAGTTCCAGACACCTCCCAAGC |
| | | ACGCAGCAATGCAGCTCAAAACGCTTAGCCTAGCCACACCCCCACGGGAAACAGCAGTGATTAACCTTTAGC |
| | | AATAAACGAAAGTTTAACTAAGCTATACTAACCCCAGGGTTGGTCAATTTCGTGCCAGCCACACCCTGGAGC |
| | | TAGCAAAAAAAAAAAAAAAAAAAAAAAAAAAAAAGCATATGACTAAAAAAAAAAAAAAAAAAAAAAAAAAAAA |
| | | AAAAAAAAAAAAAAAAAAAAAAAAAAAAAAAAAAAAAAAAA |

TABLE 9
Description of RBP020.22 (Omicron BA.4/BA.5-specific
SARS-CoV-2 RNA vaccine) as shown in Table 8 above

| Construct | Omicron BA.4/BA.5 P2 | |
|---|---|---|
| Antigen | P2-mutated full spike protein | |
| Changes | Amino Acid^{a} | mRNA Nucleotides (location) |
| Furin site | RRAR (SEQ ID NO: 178) | CGGAGAGCCAGA (SEQ ID |
| | | NO: 179) (2082-2093) |
| Proline | K986P | CCU (3000-3002) |
| | V987P | CCU (3003-3005) |
| Lineage | T19I | AUC (108-110) |
| | L24del | / |
| | P25del | / |
| | P26del | / |
| | A27S | UCA (123-125) |
| | H69del | / |
| | V70del | / |
| | G142D | GAC (462-464) |
| | V213G | GGC (675-677) |
| | G339D | GAC (1053-1055) |
| | S371F | UUC (1149-1151) |
| | S373P | CCC (1155-1157) |
| | S375F | UUC (1161-1163) |
| | T376A | GCA (1164-1166) |
| | D405N | AAC (1251-1253) |
| | K417N | AAC (1287-1289) |
| | N440K | AAG (1356-1358) |
| | L452R | AGG (1392-1394) |
| | S477N | AAC (1467-1469) |
| | T478K | AAG (1470-1472) |
| | E484A | GCA (1488-1490) |
| | F486V | GUG (1494-1496) |
| | Q498R | AGG (1530-1532) |
| | N501Y | UAC (1539-1541) |
| | Y505H | CAC (1551-1553) |
| | D614G | GGC (1878-1880) |
| | H655Y | UAC (2001-2003) |
| | N679K | AAG (2073-2075) |
| | P681H | CAC (2079-2081) |
| | N764K | AAA (2328-2330) |
| | D796Y | UAC (2424-2426) |
| | Q954H | CAC (2898-2900) |
| | N969K | AAG (2943-2945) |

TABLE 10
Sequence of one embodiment of Omicron BA.4/BA.5-specific SARS-CoV-2 RNA vaccine

| SEQ | | |
|---|---|---|
| ID | | |
| NO. | Brief Description | Sequence |
| 74 | Amino acid sequence of RNA-encoded | MFVFLVLLPLVSSQCVNLITRTQSYTNSFTRGVYYPDKVF |
| | SARS-CoV-2 S protein from an | RSSVLHSTQDLFLPFFSNVTWFHAISGTNGTKRFDNPVLP |
| | Omicron BA.4/BA.5 variant (with PRO | FNDGVYFASTEKSNIIRGWIFGTTLDSKTQSLLIVNNATN |
| | mutations at positions corresponding | VVIKVCEFQFCNDPFLDVYYHKNNKSWMESEFRVYSSANN |
| | to K986P and V987P of SEQ ID NO: 1; | CTFEYVSQPFLMDLEGKQGNFKNLREFVFKNIDGYFKIYS |
| | i.e., PRO mutations at positions 981 | KHTPINLGRDLPQGFSALEPLVDLPIGINITRFQTLLALH |
| | and 982 of SEQ ID NO: 74) | RSYLTPGDSSSGWTAGAAAYYVGYLQPRTFLLKYNENGTI |
| | | TDAVDCALDPLSETKCTLKSFTVEKGIYQTSNFRVQPTES |
| | | IVRFPNITNLCPFDEVFNATRFASVYAWNRKRISNCVADY |
| | | SVLYNFAPFFAFKCYGVSPTKLNDLCFTNVYADSFVIRGN |
| | | EVSQIAPGQTGNIADYNYKLPDDFTGCVIAWNSNKLDSKV |
| | | GGNYNYRYRLFRKSNLKPFERDISTEIYQAGNKPCNGVAG |
| | | VNCYFPLQSYGFRPTYGVGHQPYRVVVLSFELLHAPATVC |
| | | GPKKSTNLVKNKCVNFNFNGLTGTGVLTESNKKFLPFQQF |
| | | GRDIADTTDAVRDPQTLEILDITPCSFGGVSVITPGTNTS |
| | | NQVAVLYQGVNCTEVPVAIHADQLTPTWRVYSTGSNVFQT |
| | | RAGCLIGAEYVNNSYECDIPIGAGICASYQTQTKSHRRAR |
| | | SVASQSIIAYTMSLGAENSVAYSNNSIAIPTNFTISVTTE |
| | | ILPVSMTKTSVDCTMYICGDSTECSNLLLQYGSFCTQLKR |
| | | ALTGIAVEQDKNTQEVFAQVKQIYKTPPIKYFGGFNFSQI |
| | | LPDPSKPSKRSFIEDLLFNKVTLADAGFIKQYGDCLGDIA |
| | | ARDLICAQKFNGLTVLPPLLTDEMIAQYTSALLAGTITSG |
| | | WTFGAGAALQIPFAMQMAYRFNGIGVTQNVLYENQKLIAN |
| | | QFNSAIGKIQDSLSSTASALGKLQDVVNHNAQALNTLVKQ |
| | | LSSKFGAISSVLNDILSRLDPPEAEVQIDRLITGRLQSLQ |
| | | TYVTQQLIRAAEIRASANLAATKMSECVLGQSKRVDFCGK |
| | | GYHLMSFPQSAPHGVVFLHVTYVPAQEKNFTTAPAICHDG |
| | | KAHFPREGVFVSNGTHWFVTQRNFYEPQIITTDNTFVSGN |
| | | CDVVIGIVNNTVYDPLQPELDSFKEELDKYFKNHTSPDVD |
| | | LGDISGINASVVNIQKEIDRLNEVAKNLNESLIDLQELGK |
| | | YEQYIKWPWYIWLGFIAGLIAIVMVTIMLCCMTSCCSCLK |
| | | GCCSCGSCCKFDEDDSEPVLKGVKLHYT |
| 75 | RNA sequence encoding a SARS-CoV-2 | AUGUUCGUGUUCCUGGUGCUGCUGCCUCUGGUGUCCAGCC |
| | S protein from an Omicron BA.4/BA.5 | AGUGUGUGAACCUGAUCACCAGAACACAGAGCUACACCAA |
| | variant (with PRO mutations at | CAGCUUUACCAGAGGCGUGUACUACCCCGACAAGGUGUUC |
| | positions corresponding to K986P and | AGAUCCAGCGUGCUGCACUCUACCCAGGACCUGUUCCUGC |
| | V987P of SEQ ID NO: 1; i.e., PRO | CUUUCUUCAGCAACGUGACCUGGUUCCACGCCAUCUCCGG |
| | mutations at positions 981 and 982 of | CACCAAUGGCACCAAGAGAUUCGACAACCCCGUGCUGCCC |
| | SEQ ID NO: 74) | UUCAACGACGGGGUGUACUUUGCCAGCACCGAGAAGUCCA |
| | | ACAUCAUCAGAGGCUGGAUCUUCGGCACCACACUGGACAG |
| | | CAAGACCCAGAGCCUGCUGAUCGUGAACAACGCCACCAAC |
| | | GUGGUCAUCAAAGUGUGCGAGUUCCAGUUCUGCAACGACC |
| | | CCUUCCUGGACGUGUACUACCACAAGAACAACAAGAGCUG |
| | | GAUGGAAAGCGAGUUCCGGGUGUACAGCAGCGCCAACAAC |
| | | UGCACCUUCGAGUACGUGUCCCAGCCUUUCCUGAUGGACC |
| | | UGGAAGGCAAGCAGGGCAACUUCAAGAACCUGCGCGAGUU |
| | | CGUGUUUAAGAACAUCGACGGCUACUUCAAGAUCUACAGC |
| | | AAGCACACCCCUAUCAACCUCGGCCGGGAUCUGCCUCAGG |
| | | GCUUCUCUGCUCUGGAACCCCUGGUGGAUCUGCCCAUCGG |
| | | CAUCAACAUCACCCGGUUUCAGACACUGCUGGCCCUGCAC |
| | | AGAAGCUACCUGACACCUGGCGAUAGCAGCAGCGGAUGGA |
| | | CAGCUGGUGCCGCCGCUUACUAUGUGGGCUACCUGCAGCC |
| | | UAGAACCUUCCUGCUGAAGUACAACGAGAACGGCACCAUC |
| | | ACCGACGCCGUGGAUUGUGCUCUGGAUCCUCUGAGCGAGA |
| | | CAAAGUGCACCCUGAAGUCCUUCACCGUGGAAAAGGGCAU |
| | | CUACCAGACCAGCAACUUCCGGGUGCAGCCCACCGAAUCC |
| | | AUCGUGCGGUUCCCCAAUAUCACCAAUCUGUGCCCCUUCG |
| | | ACGAGGUGUUCAAUGCCACCAGAUUCGCCUCUGUGUACGC |
| | | CUGGAACCGGAAGCGGAUCAGCAAUUGCGUGGCCGACUAC |
| | | UCCGUGCUGUACAACUUCGCCCCCUUCUUCGCCUUCAAGU |
| | | GCUACGGCGUGUCCCCUACCAAGCUGAACGACCUGUGCUU |
| | | CACAAACGUGUACGCCGACAGCUUCGUGAUCCGGGGAAAC |
| | | GAAGUGAGCCAGAUUGCCCCUGGACAGACAGGCAACAUCG |
| | | CCGACUACAACUACAAGCUGCCCGACGACUUCACCGGCUG |
| | | UGUGAUUGCCUGGAACAGCAACAAGCUGGACUCCAAAGUC |
| | | GGCGGCAACUACAAUUACAGGUACCGGCUGUUCCGGAAGU |
| | | CCAAUCUGAAGCCCUUCGAGCGGGACAUCUCCACCGAGAU |
| | | CUAUCAGGCCGGCAACAAGCCUUGUAACGGCGUGGCCGGC |
| | | GUGAACUGCUACUUCCCACUGCAGUCCUACGGCUUUAGGC |
| | | CCACAUAUGGCGUGGGCCAUCAGCCCUACAGAGUGGUGGU |
| | | GCUGAGCUUCGAACUGCUGCAUGCCCCUGCCACAGUGUGC |
| | | GGCCCUAAGAAAAGCACCAAUCUCGUGAAGAACAAAUGCG |
| | | UGAACUUCAACUUCAACGGCCUGACCGGCACCGGCGUGCU |
| | | GACAGAGAGCAACAAGAAGUUCCUGCCAUUCCAGCAGUUU |
| | | GGCCGGGAUAUCGCCGAUACCACAGACGCCGUUAGAGAUC |
| | | CCCAGACACUGGAAAUCCUGGACAUCACCCCUUGCAGCUU |
| | | CGGCGGAGUGUCUGUGAUCACCCCUGGCACCAACACCAGC |
| | | AAUCAGGUGGCAGUGCUGUACCAGGGCGUGAACUGUACCG |
| | | AAGUGCCCGUGGCCAUUCACGCCGAUCAGCUGACACCUAC |
| | | AUGGCGGGUGUACUCCACCGGCAGCAAUGUGUUUCAGACC |
| | | AGAGCCGGCUGUCUGAUCGGAGCCGAGUACGUGAACAAUA |
| | | GCUACGAGUGCGACAUCCCCAUCGGCGCUGGAAUCUGCGC |
| | | CAGCUACCAGACACAGACAAAGAGCCACCGGAGAGCCAGA |
| | | AGCGUGGCCAGCCAGAGCAUCAUUGCCUACACAAUGUCUC |
| | | UGGGCGCCGAGAACAGCGUGGCCUACUCCAACAACUCUAU |
| | | CGCUAUCCCCACCAACUUCACCAUCAGCGUGACCACAGAG |
| | | AUCCUGCCUGUGUCCAUGACCAAGACCAGCGUGGACUGCA |
| | | CCAUGUACAUCUGCGGCGAUUCCACCGAGUGCUCCAACCU |
| | | GCUGCUGCAGUACGGCAGCUUCUGCACCCAGCUGAAGAGA |
| | | GCCCUGACAGGGAUCGCCGUGGAACAGGACAAGAACACCC |
| | | AAGAGGUGUUCGCCCAAGUGAAGCAGAUCUACAAGACCCC |
| | | UCCUAUCAAGUACUUCGGCGGCUUCAAUUUCAGCCAGAUU |
| | | CUGCCCGAUCCUAGCAAGCCCAGCAAGCGGAGCUUCAUCG |
| | | AGGACCUGCUGUUCAACAAAGUGACACUGGCCGACGCCGG |
| | | CUUCAUCAAGCAGUAUGGCGAUUGUCUGGGCGACAUUGCC |
| | | GCCAGGGAUCUGAUUUGCGCCCAGAAGUUUAACGGACUGA |
| | | CAGUGCUGCCUCCUCUGCUGACCGAUGAGAUGAUCGCCCA |
| | | GUACACAUCUGCCCUGCUGGCCGGCACAAUCACAAGCGGC |
| | | UGGACAUUUGGAGCAGGCGCCGCUCUGCAGAUCCCCUUUG |
| | | CUAUGCAGAUGGCCUACCGGUUCAACGGCAUCGGAGUGAC |
| | | CCAGAAUGUGCUGUACGAGAACCAGAAGCUGAUCGCCAAC |
| | | CAGUUCAACAGCGCCAUCGGCAAGAUCCAGGACAGCCUGA |
| | | GCAGCACAGCAAGCGCCCUGGGAAAGCUGCAGGACGUGGU |
| | | CAACCACAAUGCCCAGGCACUGAACACCCUGGUCAAGCAG |
| | | CUGUCCUCCAAGUUCGGCGCCAUCAGCUCUGUGCUGAACG |
| | | AUAUCCUGAGCAGACUGGACCCUCCUGAGGCCGAGGUGCA |
| | | GAUCGACAGACUGAUCACAGGCAGACUGCAGAGCCUCCAG |
| | | ACAUACGUGACCCAGCAGCUGAUCAGAGCCGCCGAGAUUA |
| | | GAGCCUCUGCCAAUCUGGCCGCCACCAAGAUGUCUGAGUG |
| | | UGUGCUGGGCCAGAGCAAGAGAGUGGACUUUUGCGGCAAG |
| | | GGCUACCACCUGAUGAGCUUCCCUCAGUCUGCCCCUCACG |
| | | GCGUGGUGUUUCUGCACGUGACAUAUGUGCCCGCUCAAGA |
| | | GAAGAAUUUCACCACCGCUCCAGCCAUCUGCCACGACGGC |
| | | AAAGCCCACUUUCCUAGAGAAGGCGUGUUCGUGUCCAACG |
| | | GCACCCAUUGGUUCGUGACACAGCGGAACUUCUACGAGCC |
| | | CCAGAUCAUCACCACCGACAACACCUUCGUGUCUGGCAAC |
| | | UGCGACGUCGUGAUCGGCAUUGUGAACAAUACCGUGUACG |
| | | ACCCUCUGCAGCCCGAGCUGGACAGCUUCAAAGAGGAACU |
| | | GGACAAGUACUUUAAGAACCACACAAGCCCCGACGUGGAC |
| | | CUGGGCGAUAUCAGCGGAAUCAAUGCCAGCGUCGUGAACA |
| | | UCCAGAAAGAGAUCGACCGGCUGAACGAGGUGGCCAAGAA |
| | | UCUGAACGAGAGCCUGAUCGACCUGCAAGAACUGGGGAAG |
| | | UACGAGCAGUACAUCAAGUGGCCCUGGUACAUCUGGCUGG |
| | | GCUUUAUCGCCGGACUGAUUGCCAUCGUGAUGGUCACAAU |
| | | CAUGCUGUGUUGCAUGACCAGCUGCUGUAGCUGCCUGAAG |
| | | GGCUGUUGUAGCUGUGGCAGCUGCUGCAAGUUCGACGAGG |
| | | ACGAUUCUGAGCCCGUGCUGAAGGGCGUGAAACUGCACUA |
| | | CACAUGAUGA |
| 76 | DNA sequence encoding a SARS-CoV-2 | ATGTTCGTGTTCCTGGTGCTGCTGCCTCTGGTGTCCAGCC |
| | S protein from an Omicron BA.4/BA.5 | AGTGTGTGAACCTGATCACCAGAACACAGAGCTACACCAA |
| | variant (with proline residues at | CAGCTTTACCAGAGGCGTGTACTACCCCGACAAGGTGTTC |
| | positions corresponding to K986P and | AGATCCAGCGTGCTGCACTCTACCCAGGACCTGTTCCTGC |
| | V987P of SEQ ID NO: 1) | CTTTCTTCAGCAACGTGACCTGGTTCCACGCCATCTCCGG |
| | | CACCAATGGCACCAAGAGATTCGACAACCCCGTGCTGCCC |
| | | TTCAACGACGGGGTGTACTTTGCCAGCACCGAGAAGTCCA |
| | | ACATCATCAGAGGCTGGATCTTCGGCACCACACTGGACAG |
| | | CAAGACCCAGAGCCTGCTGATCGTGAACAACGCCACCAAC |
| | | GTGGTCATCAAAGTGTGCGAGTTCCAGTTCTGCAACGACC |
| | | CCTTCCTGGACGTGTACTACCACAAGAACAACAAGAGCTG |
| | | GATGGAAAGCGAGTTCCGGGTGTACAGCAGCGCCAACAAC |
| | | TGCACCTTCGAGTACGTGTCCCAGCCTTTCCTGATGGACC |
| | | TGGAAGGCAAGCAGGGCAACTTCAAGAACCTGCGCGAGTT |
| | | CGTGTTTAAGAACATCGACGGCTACTTCAAGATCTACAGC |
| | | AAGCACACCCCTATCAACCTCGGCCGGGATCTGCCTCAGG |
| | | GCTTCTCTGCTCTGGAACCCCTGGTGGATCTGCCCATCGG |
| | | CATCAACATCACCCGGTTTCAGACACTGCTGGCCCTGCAC |
| | | AGAAGCTACCTGACACCTGGCGATAGCAGCAGCGGATGGA |
| | | CAGCTGGTGCCGCCGCTTACTATGTGGGCTACCTGCAGCC |
| | | TAGAACCTTCCTGCTGAAGTACAACGAGAACGGCACCATC |
| | | ACCGACGCCGTGGATTGTGCTCTGGATCCTCTGAGCGAGA |
| | | CAAAGTGCACCCTGAAGTCCTTCACCGTGGAAAAGGGCAT |
| | | CTACCAGACCAGCAACTTCCGGGTGCAGCCCACCGAATCC |
| | | ATCGTGCGGTTCCCCAATATCACCAATCTGTGCCCCTTCG |
| | | ACGAGGTGTTCAATGCCACCAGATTCGCCTCTGTGTACGC |
| | | CTGGAACCGGAAGCGGATCAGCAATTGCGTGGCCGACTAC |
| | | TCCGTGCTGTACAACTTCGCCCCCTTCTTCGCCTTCAAGT |
| | | GCTACGGCGTGTCCCCTACCAAGCTGAACGACCTGTGCTT |
| | | CACAAACGTGTACGCCGACAGCTTCGTGATCCGGGGAAAC |
| | | GAAGTGAGCCAGATTGCCCCTGGACAGACAGGCAACATCG |
| | | CCGACTACAACTACAAGCTGCCCGACGACTTCACCGGCTG |
| | | TGTGATTGCCTGGAACAGCAACAAGCTGGACTCCAAAGTC |
| | | GGCGGCAACTACAATTACAGGTACCGGCTGTTCCGGAAGT |
| | | CCAATCTGAAGCCCTTCGAGCGGGACATCTCCACCGAGAT |
| | | CTATCAGGCCGGCAACAAGCCTTGTAACGGCGTGGCCGGC |
| | | GTGAACTGCTACTTCCCACTGCAGTCCTACGGCTTTAGGC |
| | | CCACATATGGCGTGGGCCATCAGCCCTACAGAGTGGTGGT |
| | | GCTGAGCTTCGAACTGCTGCATGCCCCTGCCACAGTGTGC |
| | | GGCCCTAAGAAAAGCACCAATCTCGTGAAGAACAAATGCG |
| | | TGAACTTCAACTTCAACGGCCTGACCGGCACCGGCGTGCT |
| | | GACAGAGAGCAACAAGAAGTTCCTGCCATTCCAGCAGTTT |
| | | GGCCGGGATATCGCCGATACCACAGACGCCGTTAGAGATC |
| | | CCCAGACACTGGAAATCCTGGACATCACCCCTTGCAGCTT |
| | | CGGCGGAGTGTCTGTGATCACCCCTGGCACCAACACCAGC |
| | | AATCAGGTGGCAGTGCTGTACCAGGGCGTGAACTGTACCG |
| | | AAGTGCCCGTGGCCATTCACGCCGATCAGCTGACACCTAC |
| | | ATGGCGGGTGTACTCCACCGGCAGCAATGTGTTTCAGACC |
| | | AGAGCCGGCTGTCTGATCGGAGCCGAGTACGTGAACAATA |
| | | GCTACGAGTGCGACATCCCCATCGGCGCTGGAATCTGCGC |
| | | CAGCTACCAGACACAGACAAAGAGCCACCGGAGAGCCAGA |
| | | AGCGTGGCCAGCCAGAGCATCATTGCCTACACAATGTCTC |
| | | TGGGCGCCGAGAACAGCGTGGCCTACTCCAACAACTCTAT |
| | | CGCTATCCCCACCAACTTCACCATCAGCGTGACCACAGAG |
| | | ATCCTGCCTGTGTCCATGACCAAGACCAGCGTGGACTGCA |
| | | CCATGTACATCTGCGGCGATTCCACCGAGTGCTCCAACCT |
| | | GCTGCTGCAGTACGGCAGCTTCTGCACCCAGCTGAAGAGA |
| | | GCCCTGACAGGGATCGCCGTGGAACAGGACAAGAACACCC |
| | | AAGAGGTGTTCGCCCAAGTGAAGCAGATCTACAAGACCCC |
| | | TCCTATCAAGTACTTCGGCGGCTTCAATTTCAGCCAGATT |
| | | CTGCCCGATCCTAGCAAGCCCAGCAAGCGGAGCTTCATCG |
| | | AGGACCTGCTGTTCAACAAAGTGACACTGGCCGACGCCGG |
| | | CTTCATCAAGCAGTATGGCGATTGTCTGGGCGACATTGCC |
| | | GCCAGGGATCTGATTTGCGCCCAGAAGTTTAACGGACTGA |
| | | CAGTGCTGCCTCCTCTGCTGACCGATGAGATGATCGCCCA |
| | | GTACACATCTGCCCTGCTGGCCGGCACAATCACAAGCGGC |
| | | TGGACATTTGGAGCAGGCGCCGCTCTGCAGATCCCCTTTG |
| | | CTATGCAGATGGCCTACCGGTTCAACGGCATCGGAGTGAC |
| | | CCAGAATGTGCTGTACGAGAACCAGAAGCTGATCGCCAAC |
| | | CAGTTCAACAGCGCCATCGGCAAGATCCAGGACAGCCTGA |
| | | GCAGCACAGCAAGCGCCCTGGGAAAGCTGCAGGACGTGGT |
| | | CAACCACAATGCCCAGGCACTGAACACCCTGGTCAAGCAG |
| | | CTGTCCTCCAAGTTCGGCGCCATCAGCTCTGTGCTGAACG |
| | | ATATCCTGAGCAGACTGGACCCTCCTGAGGCCGAGGTGCA |
| | | GATCGACAGACTGATCACAGGCAGACTGCAGAGCCTCCAG |
| | | ACATACGTGACCCAGCAGCTGATCAGAGCCGCCGAGATTA |
| | | GAGCCTCTGCCAATCTGGCCGCCACCAAGATGTCTGAGTG |
| | | TGTGCTGGGCCAGAGCAAGAGAGTGGACTTTTGCGGCAAG |
| | | GGCTACCACCTGATGAGCTTCCCTCAGTCTGCCCCTCACG |
| | | GCGTGGTGTTTCTGCACGTGACATATGTGCCCGCTCAAGA |
| | | GAAGAATTTCACCACCGCTCCAGCCATCTGCCACGACGGC |
| | | AAAGCCCACTTTCCTAGAGAAGGCGTGTTCGTGTCCAACG |
| | | GCACCCATTGGTTCGTGACACAGCGGAACTTCTACGAGCC |
| | | CCAGATCATCACCACCGACAACACCTTCGTGTCTGGCAAC |
| | | TGCGACGTCGTGATCGGCATTGTGAACAATACCGTGTACG |
| | | ACCCTCTGCAGCCCGAGCTGGACAGCTTCAAAGAGGAACT |
| | | GGACAAGTACTTTAAGAACCACACAAGCCCCGACGTGGAC |
| | | CTGGGCGATATCAGCGGAATCAATGCCAGCGTCGTGAACA |
| | | TCCAGAAAGAGATCGACCGGCTGAACGAGGTGGCCAAGAA |
| | | TCTGAACGAGAGCCTGATCGACCTGCAAGAACTGGGGAAG |
| | | TACGAGCAGTACATCAAGTGGCCCTGGTACATCTGGCTGG |
| | | GCTTTATCGCCGGACTGATTGCCATCGTGATGGTCACAAT |
| | | CATGCTGTGTTGCATGACCAGCTGCTGTAGCTGCCTGAAG |
| | | GGCTGTTGTAGCTGTGGCAGCTGCTGCAAGTTCGACGAGG |
| | | ACGATTCTGAGCCCGTGCTGAAGGGCGTGAAACTGCACTA |
| | | CACATGATGA |

TABLE 11
Sequence of one embodiment of Omicron BA.4/BA.5-specific SARS-CoV-2 RNA vaccine

| SEQ | | |
|---|---|---|
| ID | | |
| NO. | Brief Description | Sequence |
| 77 | Amino acid sequence of RNA-encoded | MFVFLVLLPLVSSQCVNLITRTQSYTNSFTRGVYYPDKVF |
| | SARS-CoV-2 S protein from an | RSSVLHSTQDLFLPFFSNVTWFHAISGTNGTKRFDNPVLP |
| | Omicron BA.4/BA.5 variant (with PRO | FNDGVYFASTEKSNIIRGWIFGTTLDSKTQSLLIVNNATN |
| | mutations at positions corresponding | VVIKVCEFQFCNDPFLDVYYHKNNKSWMESEFRVYSSANN |
| | to K986P and V987P of SEQ ID NO: 1; | CTFEYVSQPFLMDLEGKQGNFKNLREFVFKNIDGYFKIYS |
| | i.e., PRO mutations at positions 981 | KHTPINLGRDLPQGFSALEPLVDLPIGINITRFQTLLALH |
| | and 982 of SEQ ID NO: 77) | RSYLTPGDSSSGWTAGAAAYYVGYLQPRTFLLKYNENGTI |
| | | TDAVDCALDPLSETKCTLKSFTVEKGIYQTSNFRVQPTES |
| | | IVRFPNITNLCPFDEVFNATRFASVYAWNRKRISNCVADY |
| | | SVLYNFAPFFAFKCYGVSPTKLNDLCFTNVYADSFVIRGN |
| | | EVRQIAPGQTGNIADYNYKLPDDFTGCVIAWNSNKLDSKV |
| | | GGNYNYRYRLFRKSNLKPFERDISTEIYQAGNKPCNGVAG |
| | | VNCYFPLQSYGFRPTYGVGHQPYRVVVLSFELLHAPATVC |
| | | GPKKSTNLVKNKCVNFNFNGLTGTGVLTESNKKFLPFQQF |
| | | GRDIADTTDAVRDPQTLEILDITPCSFGGVSVITPGTNTS |
| | | NQVAVLYQGVNCTEVPVAIHADQLTPTWRVYSTGSNVFQT |
| | | RAGCLIGAEYVNNSYECDIPIGAGICASYQTQTKSHRRAR |
| | | SVASQSIIAYTMSLGAENSVAYSNNSIAIPTNFTISVTTE |
| | | ILPVSMTKTSVDCTMYICGDSTECSNLLLQYGSFCTQLKR |
| | | ALTGIAVEQDKNTQEVFAQVKQIYKTPPIKYFGGFNFSQI |
| | | LPDPSKPSKRSFIEDLLFNKVTLADAGFIKQYGDCLGDIA |
| | | ARDLICAQKFNGLTVLPPLLTDEMIAQYTSALLAGTITSG |
| | | WTFGAGAALQIPFAMQMAYRFNGIGVTQNVLYENQKLIAN |
| | | QFNSAIGKIQDSLSSTASALGKLQDVVNHNAQALNTLVKQ |
| | | LSSKFGAISSVLNDILSRLDPPEAEVQIDRLITGRLQSLQ |
| | | TYVTQQLIRAAEIRASANLAATKMSECVLGQSKRVDFCGK |
| | | GYHLMSFPQSAPHGVVFLHVTYVPAQEKNFTTAPAICHDG |
| | | KAHFPREGVFVSNGTHWFVTQRNFYEPQIITTDNTFVSGN |
| | | CDVVIGIVNNTVYDPLQPELDSFKEELDKYFKNHTSPDVD |
| | | LGDISGINASVVNIQKEIDRLNEVAKNLNESLIDLQELGK |
| | | YEQYIKWPWYIWLGFIAGLIAIVMVTIMLCCMTSCCSCLK |
| | | GCCSCGSCCKFDEDDSEPVLKGVKLHYT |
| 78 | RNA sequence encoding a SARS-CoV-2 | AUGUUCGUGUUCCUGGUGCUGCUGCCUCUGGUGUCCAGCC |
| | S protein from an Omicron BA.4/BA.5 | AGUGUGUGAACCUGAUCACCAGAACACAGUCAUACACCAA |
| | variant | CAGCUUUACCAGAGGCGUGUACUACCCCGACAAGGUGUUC |
| | | AGAUCCAGCGUGCUGCACUCUACCCAGGACCUGUUCCUGC |
| | | CUUUCUUCAGCAACGUGACCUGGUUCCACGCCAUCUCCGG |
| | | CACCAAUGGCACCAAGAGAUUCGACAACCCCGUGCUGCCC |
| | | UUCAACGACGGGGUGUACUUUGCCAGCACCGAGAAGUCCA |
| | | ACAUCAUCAGAGGCUGGAUCUUCGGCACCACACUGGACAG |
| | | CAAGACCCAGAGCCUGCUGAUCGUGAACAACGCCACCAAC |
| | | GUGGUCAUCAAAGUGUGCGAGUUCCAGUUCUGCAACGACC |
| | | CCUUCCUGGACGUCUACUACCACAAGAACAACAAGAGCUG |
| | | GAUGGAAAGCGAGUUCCGGGUGUACAGCAGCGCCAACAAC |
| | | UGCACCUUCGAGUACGUGUCCCAGCCUUUCCUGAUGGACC |
| | | UGGAAGGCAAGCAGGGCAACUUCAAGAACCUGCGCGAGUU |
| | | CGUGUUUAAGAACAUCGACGGCUACUUCAAGAUCUACAGC |
| | | AAGCACACCCCUAUCAACCUCGGCCGGGAUCUGCCUCAGG |
| | | GCUUCUCUGCUCUGGAACCCCUGGUGGAUCUGCCCAUCGG |
| | | CAUCAACAUCACCCGGUUUCAGACACUGCUGGCCCUGCAC |
| | | AGAAGCUACCUGACACCUGGCGAUAGCAGCAGCGGAUGGA |
| | | CAGCUGGUGCCGCCGCUUACUAUGUGGGCUACCUGCAGCC |
| | | UAGAACCUUCCUGCUGAAGUACAACGAGAACGGCACCAUC |
| | | ACCGACGCCGUGGAUUGUGCUCUGGAUCCUCUGAGCGAGA |
| | | CAAAGUGCACCCUGAAGUCCUUCACCGUGGAAAAGGGCAU |
| | | CUACCAGACCAGCAACUUCCGGGUGCAGCCCACCGAAUCC |
| | | AUCGUGCGGUUCCCCAAUAUCACCAAUCUGUGCCCCUUCG |
| | | ACGAGGUGUUCAAUGCCACCAGAUUCGCCUCUGUGUACGC |
| | | CUGGAACCGGAAGCGGAUCAGCAAUUGCGUGGCCGACUAC |
| | | UCCGUGCUGUACAACUUCGCCCCCUUCUUCGCAUUCAAGU |
| | | GCUACGGCGUGUCCCCUACCAAGCUGAACGACCUGUGCUU |
| | | CACAAACGUGUACGCCGACAGCUUCGUGAUCCGGGGAAAC |
| | | GAAGUGCGGCAGAUUGCCCCUGGACAGACAGGCAACAUCG |
| | | CCGACUACAACUACAAGCUGCCCGACGACUUCACCGGCUG |
| | | UGUGAUUGCCUGGAACAGCAACAAGCUGGACUCCAAAGUC |
| | | GGCGGCAACUACAAUUACAGGUACCGGCUGUUCCGGAAGU |
| | | CCAAUCUGAAGCCCUUCGAGCGGGACAUCUCCACCGAGAU |
| | | CUAUCAGGCCGGCAACAAGCCUUGUAACGGCGUGGCAGGC |
| | | GUGAACUGCUACUUCCCACUGCAGUCCUACGGCUUUAGGC |
| | | CCACAUACGGCGUGGGCCACCAGCCCUACAGAGUGGUGGU |
| | | GCUGAGCUUCGAACUGCUGCAUGCCCCUGCCACAGUGUGC |
| | | GGCCCUAAGAAAAGCACCAAUCUCGUGAAGAACAAAUGCG |
| | | UGAACUUCAACUUCAACGGCCUGACCGGCACCGGCGUGCU |
| | | GACAGAGAGCAACAAGAAGUUCCUGCCAUUCCAGCAGUUU |
| | | GGCCGGGAUAUCGCCGAUACCACAGACGCCGUUAGAGAUC |
| | | CCCAGACACUGGAAAUCCUGGACAUCACCCCUUGCAGCUU |
| | | CGGCGGAGUGUCUGUGAUCACCCCUGGCACCAACACCAGC |
| | | AAUCAGGUGGCAGUGCUGUACCAGGGCGUGAACUGUACCG |
| | | AAGUGCCCGUGGCCAUUCACGCCGAUCAGCUGACACCUAC |
| | | AUGGCGGGUGUACUCCACCGGCAGCAAUGUGUUUCAGACC |
| | | AGAGCCGGCUGUCUGAUCGGAGCCGAGUACGUGAACAAUA |
| | | GCUACGAGUGCGACAUCCCCAUCGGCGCUGGAAUCUGCGC |
| | | CAGCUACCAGACACAGACAAAGAGCCACCGGAGAGCCAGA |
| | | AGCGUGGCCAGCCAGAGCAUCAUUGCCUACACAAUGUCUC |
| | | UGGGCGCCGAGAACAGCGUGGCCUACUCCAACAACUCUAU |
| | | CGCUAUCCCCACCAACUUCACCAUCAGCGUGACCACAGAG |
| | | AUCCUGCCUGUGUCCAUGACCAAGACCAGCGUGGACUGCA |
| | | CCAUGUACAUCUGCGGCGAUUCCACCGAGUGCUCCAACCU |
| | | GCUGCUGCAGUACGGCAGCUUCUGCACCCAGCUGAAAAGA |
| | | GCCCUGACAGGGAUCGCCGUGGAACAGGACAAGAACACCC |
| | | AAGAGGUGUUCGCCCAAGUGAAGCAGAUCUACAAGACCCC |
| | | UCCUAUCAAGUACUUCGGCGGCUUCAAUUUCAGCCAGAUU |
| | | CUGCCCGAUCCUAGCAAGCCCAGCAAGCGGAGCUUCAUCG |
| | | AGGACCUGCUGUUCAACAAAGUGACACUGGCCGACGCCGG |
| | | CUUCAUCAAGCAGUAUGGCGAUUGUCUGGGCGACAUUGCC |
| | | GCCAGGGAUCUGAUUUGCGCCCAGAAGUUUAACGGACUGA |
| | | CAGUGCUGCCUCCUCUGCUGACCGAUGAGAUGAUCGCCCA |
| | | GUACACAUCUGCCCUGCUGGCCGGCACAAUCACAAGCGGC |
| | | UGGACAUUUGGAGCAGGCGCCGCUCUGCAGAUCCCCUUUG |
| | | CUAUGCAGAUGGCCUACCGGUUCAACGGCAUCGGAGUGAC |
| | | CCAGAAUGUGCUGUACGAGAACCAGAAGCUGAUCGCCAAC |
| | | CAGUUCAACAGCGCCAUCGGCAAGAUCCAGGACAGCCUGA |
| | | GCAGCACAGCAAGCGCCCUGGGAAAGCUGCAGGACGUGGU |
| | | CAACCACAAUGCCCAGGCACUGAACACCCUGGUCAAGCAG |
| | | CUGUCCUCCAAGUUCGGCGCCAUCAGCUCUGUGCUGAACG |
| | | AUAUCCUGAGCAGACUGGACCCUCCUGAGGCCGAGGUGCA |
| | | GAUCGACAGACUGAUCACAGGCAGACUGCAGAGCCUCCAG |
| | | ACAUACGUGACCCAGCAGCUGAUCAGAGCCGCCGAGAUUA |
| | | GAGCCUCUGCCAAUCUGGCCGCCACCAAGAUGUCUGAGUG |
| | | UGUGCUGGGCCAGAGCAAGAGAGUGGACUUUUGCGGCAAG |
| | | GGCUACCACCUGAUGAGCUUCCCUCAGUCUGCCCCUCACG |
| | | GCGUGGUGUUUCUGCACGUGACAUAUGUGCCCGCUCAAGA |
| | | GAAGAAUUUCACCACCGCUCCAGCCAUCUGCCACGACGGC |
| | | AAAGCCCACUUUCCUAGAGAAGGCGUGUUCGUGUCCAACG |
| | | GCACCCAUUGGUUCGUGACACAGCGGAACUUCUACGAGCC |
| | | CCAGAUCAUCACCACCGACAACACCUUCGUGUCUGGCAAC |
| | | UGCGACGUCGUGAUCGGCAUUGUGAACAAUACCGUGUACG |
| | | ACCCUCUGCAGCCCGAGCUGGACAGCUUCAAAGAGGAACU |
| | | GGACAAGUACUUUAAGAACCACACAAGCCCCGACGUGGAC |
| | | CUGGGCGAUAUCAGCGGAAUCAAUGCCAGCGUCGUGAACA |
| | | UCCAGAAAGAGAUCGACCGGCUGAACGAGGUGGCCAAGAA |
| | | UCUGAACGAGAGCCUGAUCGACCUGCAAGAACUGGGGAAG |
| | | UACGAGCAGUACAUCAAGUGGCCCUGGUACAUCUGGCUGG |
| | | GCUUUAUCGCCGGACUGAUUGCCAUCGUGAUGGUCACAAU |
| | | CAUGCUGUGUUGCAUGACCAGCUGCUGUAGCUGCCUGAAG |
| | | GGCUGUUGUAGCUGUGGCAGCUGCUGCAAGUUCGACGAGG |
| | | ACGAUUCUGAGCCCGUGCUGAAGGGCGUGAAACUGCACUA |
| | | CACAUGAUGA |
| 79 | DNA sequence encoding a SARS-CoV-2 | ATGTTCGTGTTCCTGGTGCTGCTGCCTCTGGTGTCCAGCC |
| | S protein from an Omicron BA.4/BA.5 | AGTGTGTGAACCTGATCACCAGAACACAGTCATACACCAA |
| | variant | CAGCTTTACCAGAGGCGTGTACTACCCCGACAAGGTGTTC |
| | | AGATCCAGCGTGCTGCACTCTACCCAGGACCTGTTCCTGC |
| | | CTTTCTTCAGCAACGTGACCTGGTTCCACGCCATCTCCGG |
| | | CACCAATGGCACCAAGAGATTCGACAACCCCGTGCTGCCC |
| | | TTCAACGACGGGGTGTACTTTGCCAGCACCGAGAAGTCCA |
| | | ACATCATCAGAGGCTGGATCTTCGGCACCACACTGGACAG |
| | | CAAGACCCAGAGCCTGCTGATCGTGAACAACGCCACCAAC |
| | | GTGGTCATCAAAGTGTGCGAGTTCCAGTTCTGCAACGACC |
| | | CCTTCCTGGACGTCTACTACCACAAGAACAACAAGAGCTG |
| | | GATGGAAAGCGAGTTCCGGGTGTACAGCAGCGCCAACAAC |
| | | TGCACCTTCGAGTACGTGTCCCAGCCTTTCCTGATGGACC |
| | | TGGAAGGCAAGCAGGGCAACTTCAAGAACCTGCGCGAGTT |
| | | CGTGTTTAAGAACATCGACGGCTACTTCAAGATCTACAGC |
| | | AAGCACACCCCTATCAACCTCGGCCGGGATCTGCCTCAGG |
| | | GCTTCTCTGCTCTGGAACCCCTGGTGGATCTGCCCATCGG |
| | | CATCAACATCACCCGGTTTCAGACACTGCTGGCCCTGCAC |
| | | AGAAGCTACCTGACACCTGGCGATAGCAGCAGCGGATGGA |
| | | CAGCTGGTGCCGCCGCTTACTATGTGGGCTACCTGCAGCC |
| | | TAGAACCTTCCTGCTGAAGTACAACGAGAACGGCACCATC |
| | | ACCGACGCCGTGGATTGTGCTCTGGATCCTCTGAGCGAGA |
| | | CAAAGTGCACCCTGAAGTCCTTCACCGTGGAAAAGGGCAT |
| | | CTACCAGACCAGCAACTTCCGGGTGCAGCCCACCGAATCC |
| | | ATCGTGCGGTTCCCCAATATCACCAATCTGTGCCCCTTCG |
| | | ACGAGGTGTTCAATGCCACCAGATTCGCCTCTGTGTACGC |
| | | CTGGAACCGGAAGCGGATCAGCAATTGCGTGGCCGACTAC |
| | | TCCGTGCTGTACAACTTCGCCCCCTTCTTCGCATTCAAGT |
| | | GCTACGGCGTGTCCCCTACCAAGCTGAACGACCTGTGCTT |
| | | CACAAACGTGTACGCCGACAGCTTCGTGATCCGGGGAAAC |
| | | GAAGTGCGGCAGATTGCCCCTGGACAGACAGGCAACATCG |
| | | CCGACTACAACTACAAGCTGCCCGACGACTTCACCGGCTG |
| | | TGTGATTGCCTGGAACAGCAACAAGCTGGACTCCAAAGTC |
| | | GGCGGCAACTACAATTACAGGTACCGGCTGTTCCGGAAGT |
| | | CCAATCTGAAGCCCTTCGAGCGGGACATCTCCACCGAGAT |
| | | CTATCAGGCCGGCAACAAGCCTTGTAACGGCGTGGCAGGC |
| | | GTGAACTGCTACTTCCCACTGCAGTCCTACGGCTTTAGGC |
| | | CCACATACGGCGTGGGCCACCAGCCCTACAGAGTGGTGGT |
| | | GCTGAGCTTCGAACTGCTGCATGCCCCTGCCACAGTGTGC |
| | | GGCCCTAAGAAAAGCACCAATCTCGTGAAGAACAAATGCG |
| | | TGAACTTCAACTTCAACGGCCTGACCGGCACCGGCGTGCT |
| | | GACAGAGAGCAACAAGAAGTTCCTGCCATTCCAGCAGTTT |
| | | GGCCGGGATATCGCCGATACCACAGACGCCGTTAGAGATC |
| | | CCCAGACACTGGAAATCCTGGACATCACCCCTTGCAGCTT |
| | | CGGCGGAGTGTCTGTGATCACCCCTGGCACCAACACCAGC |
| | | AATCAGGTGGCAGTGCTGTACCAGGGCGTGAACTGTACCG |
| | | AAGTGCCCGTGGCCATTCACGCCGATCAGCTGACACCTAC |
| | | ATGGCGGGTGTACTCCACCGGCAGCAATGTGTTTCAGACC |
| | | AGAGCCGGCTGTCTGATCGGAGCCGAGTACGTGAACAATA |
| | | GCTACGAGTGCGACATCCCCATCGGCGCTGGAATCTGCGC |
| | | CAGCTACCAGACACAGACAAAGAGCCACCGGAGAGCCAGA |
| | | AGCGTGGCCAGCCAGAGCATCATTGCCTACACAATGTCTC |
| | | TGGGCGCCGAGAACAGCGTGGCCTACTCCAACAACTCTAT |
| | | CGCTATCCCCACCAACTTCACCATCAGCGTGACCACAGAG |
| | | ATCCTGCCTGTGTCCATGACCAAGACCAGCGTGGACTGCA |
| | | CCATGTACATCTGCGGCGATTCCACCGAGTGCTCCAACCT |
| | | GCTGCTGCAGTACGGCAGCTTCTGCACCCAGCTGAAAAGA |
| | | GCCCTGACAGGGATCGCCGTGGAACAGGACAAGAACACCC |
| | | AAGAGGTGTTCGCCCAAGTGAAGCAGATCTACAAGACCCC |
| | | TCCTATCAAGTACTTCGGCGGCTTCAATTTCAGCCAGATT |
| | | CTGCCCGATCCTAGCAAGCCCAGCAAGCGGAGCTTCATCG |
| | | AGGACCTGCTGTTCAACAAAGTGACACTGGCCGACGCCGG |
| | | CTTCATCAAGCAGTATGGCGATTGTCTGGGCGACATTGCC |
| | | GCCAGGGATCTGATTTGCGCCCAGAAGTTTAACGGACTGA |
| | | CAGTGCTGCCTCCTCTGCTGACCGATGAGATGATCGCCCA |
| | | GTACACATCTGCCCTGCTGGCCGGCACAATCACAAGCGGC |
| | | TGGACATTTGGAGCAGGCGCCGCTCTGCAGATCCCCTTTG |
| | | CTATGCAGATGGCCTACCGGTTCAACGGCATCGGAGTGAC |
| | | CCAGAATGTGCTGTACGAGAACCAGAAGCTGATCGCCAAC |
| | | CAGTTCAACAGCGCCATCGGCAAGATCCAGGACAGCCTGA |
| | | GCAGCACAGCAAGCGCCCTGGGAAAGCTGCAGGACGTGGT |
| | | CAACCACAATGCCCAGGCACTGAACACCCTGGTCAAGCAG |
| | | CTGTCCTCCAAGTTCGGCGCCATCAGCTCTGTGCTGAACG |
| | | ATATCCTGAGCAGACTGGACCCTCCTGAGGCCGAGGTGCA |
| | | GATCGACAGACTGATCACAGGCAGACTGCAGAGCCTCCAG |
| | | ACATACGTGACCCAGCAGCTGATCAGAGCCGCCGAGATTA |
| | | GAGCCTCTGCCAATCTGGCCGCCACCAAGATGTCTGAGTG |
| | | TGTGCTGGGCCAGAGCAAGAGAGTGGACTTTTGCGGCAAG |
| | | GGCTACCACCTGATGAGCTTCCCTCAGTCTGCCCCTCACG |
| | | GCGTGGTGTTTCTGCACGTGACATATGTGCCCGCTCAAGA |
| | | GAAGAATTTCACCACCGCTCCAGCCATCTGCCACGACGGC |
| | | AAAGCCCACTTTCCTAGAGAAGGCGTGTTCGTGTCCAACG |
| | | GCACCCATTGGTTCGTGACACAGCGGAACTTCTACGAGCC |
| | | CCAGATCATCACCACCGACAACACCTTCGTGTCTGGCAAC |
| | | TGCGACGTCGTGATCGGCATTGTGAACAATACCGTGTACG |
| | | ACCCTCTGCAGCCCGAGCTGGACAGCTTCAAAGAGGAACT |
| | | GGACAAGTACTTTAAGAACCACACAAGCCCCGACGTGGAC |
| | | CTGGGCGATATCAGCGGAATCAATGCCAGCGTCGTGAACA |
| | | TCCAGAAAGAGATCGACCGGCTGAACGAGGTGGCCAAGAA |
| | | TCTGAACGAGAGCCTGATCGACCTGCAAGAACTGGGGAAG |
| | | TACGAGCAGTACATCAAGTGGCCCTGGTACATCTGGCTGG |
| | | GCTTTATCGCCGGACTGATTGCCATCGTGATGGTCACAAT |
| | | CATGCTGTGTTGCATGACCAGCTGCTGTAGCTGCCTGAAG |
| | | GGCTGTTGTAGCTGTGGCAGCTGCTGCAAGTTCGACGAGG |
| | | ACGATTCTGAGCCCGTGCTGAAGGGCGTGAAACTGCACTA |
| | | CACATGATGA |

TABLE 12
Sequence of one embodiment of an exemplary Omicron BA.2.75-specific RNA vaccine

| SEQ | | |
|---|---|---|
| ID | | |
| NO. | Brief Description | Sequence |
| 149 | Amino acid sequence of RNA-encoded | MFVFLVLLPLVSSQCVNLITRTQSYTNSFTRGVYYPDKVF |
| | SARS-CoV-2 S protein from an | RSSVLHSTQDLFLPFFSNVTWFHAIHVSGTNGTKRFDNPV |
| | Omicron BA.2.75 variant (with PRO | LPFNDGVYFASTEKSNIIRGWIFGTTLDSKTQSLLIVNNA |
| | mutations at positions corresponding | TNVVIKVCEFQFCNDPFLDVYYHENNKSRMESELRVYSSA |
| | to positions K986P and V987P of SEQ | NNCTFEYVSQPFLMDLEGKQGNFKNLREFVFKNIDGYFKI |
| | ID NO: 1; i.e., PRO mutations at | YSKHTPVNLGRDLPQGFSALEPLVDLPIGINITRFQTLLA |
| | positions 983 and 984 of SEQ ID NO: | LHRSYLTPGDSSSSWTAGAAAYYVGYLQPRTFLLKYNENG |
| | 80) | TITDAVDCALDPLSETKCTLKSFTVEKGIYQTSNFRVQPT |
| | | ESIVRFPNITNLCPFHEVFNATRFASVYAWNRKRISNCVA |
| | | DYSVLYNFAPFFAFKCYGVSPTKLNDLCFTNVYADSFVIR |
| | | GNEVSQIAPGQTGNIADYNYKLPDDFTGCVIAWNSNKLDS |
| | | KVSGNYNYLYRLFRKSKLKPFERDISTEIYQAGNKPCNGV |
| | | AGFNCYFPLQSYGFRPTYGVGHQPYRVVVLSFELLHAPAT |
| | | VCGPKKSTNLVKNKCVNFNFNGLTGTGVLTESNKKFLPFQ |
| | | QFGRDIADTTDAVRDPQTLEILDITPCSFGGVSVITPGTN |
| | | TSNQVAVLYQGVNCTEVPVAIHADQLTPTWRVYSTGSNVF |
| | | QTRAGCLIGAEYVNNSYECDIPIGAGICASYQTQTKSHRR |
| | | ARSVASQSIIAYTMSLGAENSVAYSNNSIAIPTNFTISVT |
| | | TEILPVSMTKTSVDCTMYICGDSTECSNLLLQYGSFCTQL |
| | | KRALTGIAVEQDKNTQEVFAQVKQIYKTPPIKYFGGFNFS |
| | | QILPDPSKPSKRSFIEDLLFNKVTLADAGFIKQYGDCLGD |
| | | IAARDLICAQKFNGLTVLPPLLTDEMIAQYTSALLAGTIT |
| | | SGWTFGAGAALQIPFAMQMAYRFNGIGVTQNVLYENQKLI |
| | | ANQFNSAIGKIQDSLSSTASALGKLQDVVNHNAQALNTLV |
| | | KQLSSKFGAISSVLNDILSRLDPPEAEVQIDRLITGRLQS |
| | | LQTYVTQQLIRAAEIRASANLAATKMSECVLGQSKRVDFC |
| | | GKGYHLMSFPQSAPHGVVFLHVTYVPAQEKNFTTAPAICH |
| | | DGKAHFPREGVFVSNGTHWFVTQRNFYEPQIITTDNTFVS |
| | | GNCDVVIGIVNNTVYDPLQPELDSFKEELDKYFKNHTSPD |
| | | VDLGDISGINASVVNIQKEIDRLNEVAKNLNESLIDLQEL |
| | | GKYEQYIKWPWYIWLGFIAGLIAIVMVTIMLCCMTSCCSC |
| | | LKGCCSCGSCCKFDEDDSEPVLKGVKLHYT |
| 150 | RNA sequence encoding a SARS-CoV-2 | auguucguguuccuggugcugcugccucugguguccagcc |
| | S protein from an Omicron BA.2.75 | agugugugaaccugaucaccagaacacagucauacaccaa |
| | variant | cagcuuuaccagaggcguguacuaccccgacaagguguuc |
| | | agauccagcgugcugcacucuacccaggaccuguuccugc |
| | | cuuucuucagcaacgugaccugguuccacgccauccacgu |
| | | guccggcaccaauggcaccaagagauucgacaaccccgug |
| | | cugcccuucaacgacgggguguacuuugccagcaccgaga |
| | | aguccaacaucaucagaggcuggaucuucggcaccacacu |
| | | ggacagcaagacccagagccugcugaucgugaacaacgcc |
| | | accaacguggucaucaaagugugcgaguuccaguucugca |
| | | acgaccccuuccuggacgucuacuaccacgagaacaacaa |
| | | gagcaggauggaaagcgagcuccggguguacagcagcgcc |
| | | aacaacugcaccuucgaguacgugucccagccuuuccuga |
| | | uggaccuggaaggcaagcagggcaacuucaagaaccugcg |
| | | cgaguucguguuuaagaacaucgacggcuacuucaagauc |
| | | uacagcaagcacaccccugugaaccucggccgggaucugc |
| | | cucagggcuucucugcucuggaaccccugguggaucugcc |
| | | caucggcaucaacaucacccgguuucagacacugcuggcc |
| | | cugcacagaagcuaccugacaccuggcgauagcagcagca |
| | | gcuggacagcuggugccgccgcuuacuaugugggcuaccu |
| | | gcagccuagaaccuuccugcugaaguacaacgagaacggc |
| | | accaucaccgacgccguggauugugcucuggauccucuga |
| | | gcgagacaaagugcacccugaaguccuucaccguggaaaa |
| | | gggcaucuaccagaccagcaacuuccgggugcagcccacc |
| | | gaauccaucgugcgguuccccaauaucaccaaucugugcc |
| | | ccuuccacgagguguucaaugccaccagauucgccucugu |
| | | guacgccuggaaccggaagcggaucagcaauugcguggcc |
| | | gacuacuccgugcuguacaacuucgcccccuucuucgcau |
| | | ucaagugcuacggcguguccccuaccaagcugaacgaccu |
| | | gugcuucacaaacguguacgccgacagcuucgugauccgg |
| | | ggaaacgaagugucacagauugccccuggacagacaggca |
| | | acaucgccgacuacaacuacaagcugcccgacgacuucac |
| | | cggcugugugauugccuggaacagcaacaagcuggacucc |
| | | aaagucagcggcaacuacaauuaccuguaccggcuguucc |
| | | ggaaguccaagcugaagcccuucgagcgggacaucuccac |
| | | cgagaucuaucaggccggcaacaagccuuguaacggcgug |
| | | gcaggcuucaacugcuacuucccacugcaguccuacggcu |
| | | uuaggcccacauacggcgugggccaccagcccuacagagu |
| | | gguggugcugagcuucgaacugcugcaugccccugccaca |
| | | gugugcggcccuaagaaaagcaccaaucucgugaagaaca |
| | | aaugcgugaacuucaacuucaacggccugaccggcaccgg |
| | | cgugcugacagagagcaacaagaaguuccugccauuccag |
| | | caguuuggccgggauaucgccgauaccacagacgccguua |
| | | gagauccccagacacuggaaauccuggacaucaccccuug |
| | | cagcuucggcggagugucugugaucaccccuggcaccaac |
| | | accagcaaucagguggcagugcuguaccagggcgugaacu |
| | | guaccgaagugcccguggccauucacgccgaucagcugac |
| | | accuacauggcggguguacuccaccggcagcaauguguuu |
| | | cagaccagagccggcugucugaucggagccgaguacguga |
| | | acaauagcuacgagugcgacauccccaucggcgcuggaau |
| | | cugcgccagcuaccagacacagacaaagagccaccggaga |
| | | gccagaagcguggccagccagagcaucauugccuacacaa |
| | | ugucucugggcgccgagaacagcguggccuacuccaacaa |
| | | cucuaucgcuauccccaccaacuucaccaucagcgugacc |
| | | acagagauccugccuguguccaugaccaagaccagcgugg |
| | | acugcaccauguacaucugcggcgauuccaccgagugcuc |
| | | caaccugcugcugcaguacggcagcuucugcacccagcug |
| | | aaaagagcccugacagggaucgccguggaacaggacaaga |
| | | acacccaagagguguucgcccaagugaagcagaucuacaa |
| | | gaccccuccuaucaaguacuucggcggcuucaauuucagc |
| | | cagauucugcccgauccuagcaagcccagcaagcggagcu |
| | | ucaucgaggaccugcuguucaacaaagugacacuggccga |
| | | cgccggcuucaucaagcaguauggcgauugucugggcgac |
| | | auugccgccagggaucugauuugcgcccagaaguuuaacg |
| | | gacugacagugcugccuccucugcugaccgaugagaugau |
| | | cgcccaguacacaucugcccugcuggccggcacaaucaca |
| | | agcggcuggacauuuggagcaggcgccgcucugcagaucc |
| | | ccuuugcuaugcagauggccuaccgguucaacggcaucgg |
| | | agugacccagaaugugcuguacgagaaccagaagcugauc |
| | | gccaaccaguucaacagcgccaucggcaagauccaggaca |
| | | gccugagcagcacagcaagcgcccugggaaagcugcagga |
| | | cguggucaaccacaaugcccaggcacugaacacccugguc |
| | | aagcagcuguccuccaaguucggcgccaucagcucugugc |
| | | ugaacgauauccugagcagacuggacccuccugaggccga |
| | | ggugcagaucgacagacugaucacaggcagacugcagagc |
| | | cuccagacauacgugacccagcagcugaucagagccgccg |
| | | agauuagagccucugccaaucuggccgccaccaagauguc |
| | | ugagugugugcugggccagagcaagagaguggacuuuugc |
| | | ggcaagggcuaccaccugaugagcuucccucagucugccc |
| | | cucacggcgugguguuucugcacgugacauaugugcccgc |
| | | ucaagagaagaauuucaccaccgcuccagccaucugccac |
| | | gacggcaaagcccacuuuccuagagaaggcguguucgugu |
| | | ccaacggcacccauugguucgugacacagcggaacuucua |
| | | cgagccccagaucaucaccaccgacaacaccuucgugucu |
| | | ggcaacugcgacgucgugaucggcauugugaacaauaccg |
| | | uguacgacccucugcagcccgagcuggacagcuucaaaga |
| | | ggaacuggacaaguacuuuaagaaccacacaagccccgac |
| | | guggaccugggcgauaucagcggaaucaaugccagcgucg |
| | | ugaacauccagaaagagaucgaccggcugaacgagguggc |
| | | caagaaucugaacgagagccugaucgaccugcaagaacug |
| | | gggaaguacgagcaguacaucaaguggcccugguacaucu |
| | | ggcugggcuuuaucgccggacugauugccaucgugauggu |
| | | cacaaucaugcuguguugcaugaccagcugcuguagcugc |
| | | cugaagggcuguuguagcuguggcagcugcugcaaguucg |
| | | acgaggacgauucugagcccgugcugaagggcgugaaacu |
| | | gcacuacacaugauga |
| 151 | DNA sequence encoding a SARS-CoV-2 | atgttcgtgttcctggtgctgctgcctctggtgtccagcc |
| | S protein from an Omicron BA.2.75 | agtgtgtgaacctgatcaccagaacacagtcatacaccaa |
| | variant | cagctttaccagaggcgtgtactaccccgacaaggtgttc |
| | | agatccagcgtgctgcactctacccaggacctgttcctgc |
| | | ctttcttcagcaacgtgacctggttccacgccatccacgt |
| | | gtccggcaccaatggcaccaagagattcgacaaccccgtg |
| | | ctgcccttcaacgacggggtgtactttgccagcaccgaga |
| | | agtccaacatcatcagaggctggatcttcggcaccacact |
| | | ggacagcaagacccagagcctgctgatcgtgaacaacgcc |
| | | accaacgtggtcatcaaagtgtgcgagttccagttctgca |
| | | acgaccccttcctggacgtctactaccacgagaacaacaa |
| | | gagcaggatggaaagcgagctccgggtgtacagcagcgcc |
| | | aacaactgcaccttcgagtacgtgtcccagcctttcctga |
| | | tggacctggaaggcaagcagggcaacttcaagaacctgcg |
| | | cgagttcgtgtttaagaacatcgacggctacttcaagatc |
| | | tacagcaagcacacccctgtgaacctcggccgggatctgc |
| | | ctcagggcttctctgctctggaacccctggtggatctgcc |
| | | catcggcatcaacatcacccggtttcagacactgctggcc |
| | | ctgcacagaagctacctgacacctggcgatagcagcagca |
| | | gctggacagctggtgccgccgcttactatgtgggctacct |
| | | gcagcctagaaccttcctgctgaagtacaacgagaacggc |
| | | accatcaccgacgccgtggattgtgctctggatcctctga |
| | | gcgagacaaagtgcaccctgaagtccttcaccgtggaaaa |
| | | gggcatctaccagaccagcaacttccgggtgcagcccacc |
| | | gaatccatcgtgcggttccccaatatcaccaatctgtgcc |
| | | ccttccacgaggtgttcaatgccaccagattcgcctctgt |
| | | gtacgcctggaaccggaagcggatcagcaattgcgtggcc |
| | | gactactccgtgctgtacaacttcgcccccttcttcgcat |
| | | tcaagtgctacggcgtgtcccctaccaagctgaacgacct |
| | | gtgcttcacaaacgtgtacgccgacagcttcgtgatccgg |
| | | ggaaacgaagtgtcacagattgcccctggacagacaggca |
| | | acatcgccgactacaactacaagctgcccgacgacttcac |
| | | cggctgtgtgattgcctggaacagcaacaagctggactcc |
| | | aaagtcagcggcaactacaattacctgtaccggctgttcc |
| | | ggaagtccaagctgaagcccttcgagcgggacatctccac |
| | | cgagatctatcaggccggcaacaagccttgtaacggcgtg |
| | | gcaggcttcaactgctacttcccactgcagtcctacggct |
| | | ttaggcccacatacggcgtgggccaccagccctacagagt |
| | | ggtggtgctgagcttcgaactgctgcatgcccctgccaca |
| | | gtgtgcggccctaagaaaagcaccaatctcgtgaagaaca |
| | | aatgcgtgaacttcaacttcaacggcctgaccggcaccgg |
| | | cgtgctgacagagagcaacaagaagttcctgccattccag |
| | | cagtttggccgggatatcgccgataccacagacgccgtta |
| | | gagatccccagacactggaaatcctggacatcaccccttg |
| | | cagcttcggcggagtgtctgtgatcacccctggcaccaac |
| | | accagcaatcaggtggcagtgctgtaccagggcgtgaact |
| | | gtaccgaagtgcccgtggccattcacgccgatcagctgac |
| | | acctacatggcgggtgtactccaccggcagcaatgtgttt |
| | | cagaccagagccggctgtctgatcggagccgagtacgtga |
| | | acaatagctacgagtgcgacatccccatcggcgctggaat |
| | | ctgcgccagctaccagacacagacaaagagccaccggaga |
| | | gccagaagcgtggccagccagagcatcattgcctacacaa |
| | | tgtctctgggcgccgagaacagcgtggcctactccaacaa |
| | | ctctatcgctatccccaccaacttcaccatcagcgtgacc |
| | | acagagatcctgcctgtgtccatgaccaagaccagcgtgg |
| | | actgcaccatgtacatctgcggcgattccaccgagtgctc |
| | | caacctgctgctgcagtacggcagcttctgcacccagctg |
| | | aaaagagccctgacagggatcgccgtggaacaggacaaga |
| | | acacccaagaggtgttcgcccaagtgaagcagatctacaa |
| | | gacccctcctatcaagtacttcggcggcttcaatttcagc |
| | | cagattctgcccgatcctagcaagcccagcaagcggagct |
| | | tcatcgaggacctgctgttcaacaaagtgacactggccga |
| | | cgccggcttcatcaagcagtatggcgattgtctgggcgac |
| | | attgccgccagggatctgatttgcgcccagaagtttaacg |
| | | gactgacagtgctgcctcctctgctgaccgatgagatgat |
| | | cgcccagtacacatctgccctgctggccggcacaatcaca |
| | | agcggctggacatttggagcaggcgccgctctgcagatcc |
| | | cctttgctatgcagatggcctaccggttcaacggcatcgg |
| | | agtgacccagaatgtgctgtacgagaaccagaagctgatc |
| | | gccaaccagttcaacagcgccatcggcaagatccaggaca |
| | | gcctgagcagcacagcaagcgccctgggaaagctgcagga |
| | | cgtggtcaaccacaatgcccaggcactgaacaccctggtc |
| | | aagcagctgtcctccaagttcggcgccatcagctctgtgc |
| | | tgaacgatatcctgagcagactggaccctcctgaggccga |
| | | ggtgcagatcgacagactgatcacaggcagactgcagagc |
| | | ctccagacatacgtgacccagcagctgatcagagccgccg |
| | | agattagagcctctgccaatctggccgccaccaagatgtc |
| | | tgagtgtgtgctgggccagagcaagagagtggacttttgc |
| | | ggcaagggctaccacctgatgagcttccctcagtctgccc |
| | | ctcacggcgtggtgtttctgcacgtgacatatgtgcccgc |
| | | tcaagagaagaatttcaccaccgctccagccatctgccac |
| | | gacggcaaagcccactttcctagagaaggcgtgttcgtgt |
| | | ccaacggcacccattggttcgtgacacagcggaacttcta |
| | | cgagccccagatcatcaccaccgacaacaccttcgtgtct |
| | | ggcaactgcgacgtcgtgatcggcattgtgaacaataccg |
| | | tgtacgaccctctgcagcccgagctggacagcttcaaaga |
| | | ggaactggacaagtactttaagaaccacacaagccccgac |
| | | gtggacctgggcgatatcagcggaatcaatgccagcgtcg |
| | | tgaacatccagaaagagatcgaccggctgaacgaggtggc |
| | | caagaatctgaacgagagcctgatcgacctgcaagaactg |
| | | gggaagtacgagcagtacatcaagtggccctggtacatct |
| | | ggctgggctttatcgccggactgattgccatcgtgatggt |
| | | cacaatcatgctgtgttgcatgaccagctgctgtagctgc |
| | | ctgaagggctgttgtagctgtggcagctgctgcaagttcg |
| | | acgaggacgattctgagcccgtgctgaagggcgtgaaact |
| | | gcactacacatgatga |
| 152 | Full length RNA construct encoding a | agaauaaacuaguauucuucugguccccacagacucagag |
| | SARS-CoV-2 S protein from an | agaacccgccaccauguucguguuccuggugcugcugccu |
| | Omicron BA.2.75 variant | cugguguccagccagugugugaaccugaucaccagaacac |
| | | agucauacaccaacagcuuuaccagaggcguguacuaccc |
| | | cgacaagguguucagauccagcgugcugcacucuacccag |
| | | gaccuguuccugccuuucuucagcaacgugaccugguucc |
| | | acgccauccacguguccggcaccaauggcaccaagagauu |
| | | cgacaaccccgugcugcccuucaacgacgggguguacuuu |
| | | gccagcaccgagaaguccaacaucaucagaggcuggaucu |
| | | ucggcaccacacuggacagcaagacccagagccugcugau |
| | | cgugaacaacgccaccaacguggucaucaaagugugcgag |
| | | uuccaguucugcaacgaccccuuccuggacgucuacuacc |
| | | acgagaacaacaagagcaggauggaaagcgagcuccgggu |
| | | guacagcagcgccaacaacugcaccuucgaguacgugucc |
| | | cagccuuuccugauggaccuggaaggcaagcagggcaacu |
| | | ucaagaaccugcgcgaguucguguuuaagaacaucgacgg |
| | | cuacuucaagaucuacagcaagcacaccccugugaaccuc |
| | | ggccgggaucugccucagggcuucucugcucuggaacccc |
| | | ugguggaucugcccaucggcaucaacaucacccgguuuca |
| | | gacacugcuggcccugcacagaagcuaccugacaccuggc |
| | | gauagcagcagcagcuggacagcuggugccgccgcuuacu |
| | | augugggcuaccugcagccuagaaccuuccugcugaagua |
| | | caacgagaacggcaccaucaccgacgccguggauugugcu |
| | | cuggauccucugagcgagacaaagugcacccugaaguccu |
| | | ucaccguggaaaagggcaucuaccagaccagcaacuuccg |
| | | ggugcagcccaccgaauccaucgugcgguuccccaauauc |
| | | accaaucugugccccuuccacgagguguucaaugccacca |
| | | gauucgccucuguguacgccuggaaccggaagcggaucag |
| | | caauugcguggccgacuacuccgugcuguacaacuucgcc |
| | | cccuucuucgcauucaagugcuacggcguguccccuacca |
| | | agcugaacgaccugugcuucacaaacguguacgccgacag |
| | | cuucgugauccggggaaacgaagugucacagauugccccu |
| | | ggacagacaggcaacaucgccgacuacaacuacaagcugc |
| | | ccgacgacuucaccggcugugugauugccuggaacagcaa |
| | | caagcuggacuccaaagucagcggcaacuacaauuaccug |
| | | uaccggcuguuccggaaguccaagcugaagcccuucgagc |
| | | gggacaucuccaccgagaucuaucaggccggcaacaagcc |
| | | uuguaacggcguggcaggcuucaacugcuacuucccacug |
| | | caguccuacggcuuuaggcccacauacggcgugggccacc |
| | | agcccuacagagugguggugcugagcuucgaacugcugca |
| | | ugccccugccacagugugcggcccuaagaaaagcaccaau |
| | | cucgugaagaacaaaugcgugaacuucaacuucaacggcc |
| | | ugaccggcaccggcgugcugacagagagcaacaagaaguu |
| | | ccugccauuccagcaguuuggccgggauaucgccgauacc |
| | | acagacgccguuagagauccccagacacuggaaauccugg |
| | | acaucaccccuugcagcuucggcggagugucugugaucac |
| | | cccuggcaccaacaccagcaaucagguggcagugcuguac |
| | | cagggcgugaacuguaccgaagugcccguggccauucacg |
| | | ccgaucagcugacaccuacauggcggguguacuccaccgg |
| | | cagcaauguguuucagaccagagccggcugucugaucgga |
| | | gccgaguacgugaacaauagcuacgagugcgacaucccca |
| | | ucggcgcuggaaucugcgccagcuaccagacacagacaaa |
| | | gagccaccggagagccagaagcguggccagccagagcauc |
| | | auugccuacacaaugucucugggcgccgagaacagcgugg |
| | | ccuacuccaacaacucuaucgcuauccccaccaacuucac |
| | | caucagcgugaccacagagauccugccuguguccaugacc |
| | | aagaccagcguggacugcaccauguacaucugcggcgauu |
| | | ccaccgagugcuccaaccugcugcugcaguacggcagcuu |
| | | cugcacccagcugaaaagagcccugacagggaucgccgug |
| | | gaacaggacaagaacacccaagagguguucgcccaaguga |
| | | agcagaucuacaagaccccuccuaucaaguacuucggcgg |
| | | cuucaauuucagccagauucugcccgauccuagcaagccc |
| | | agcaagcggagcuucaucgaggaccugcuguucaacaaag |
| | | ugacacuggccgacgccggcuucaucaagcaguauggcga |
| | | uugucugggcgacauugccgccagggaucugauuugcgcc |
| | | cagaaguuuaacggacugacagugcugccuccucugcuga |
| | | ccgaugagaugaucgcccaguacacaucugcccugcuggc |
| | | cggcacaaucacaagcggcuggacauuuggagcaggcgcc |
| | | gcucugcagauccccuuugcuaugcagauggccuaccggu |
| | | ucaacggcaucggagugacccagaaugugcuguacgagaa |
| | | ccagaagcugaucgccaaccaguucaacagcgccaucggc |
| | | aagauccaggacagccugagcagcacagcaagcgcccugg |
| | | gaaagcugcaggacguggucaaccacaaugcccaggcacu |
| | | gaacacccuggucaagcagcuguccuccaaguucggcgcc |
| | | aucagcucugugcugaacgauauccugagcagacuggacc |
| | | cuccugaggccgaggugcagaucgacagacugaucacagg |
| | | cagacugcagagccuccagacauacgugacccagcagcug |
| | | aucagagccgccgagauuagagccucugccaaucuggccg |
| | | ccaccaagaugucugagugugugcugggccagagcaagag |
| | | aguggacuuuugcggcaagggcuaccaccugaugagcuuc |
| | | ccucagucugccccucacggcgugguguuucugcacguga |
| | | cauaugugcccgcucaagagaagaauuucaccaccgcucc |
| | | agccaucugccacgacggcaaagcccacuuuccuagagaa |
| | | ggcguguucguguccaacggcacccauugguucgugacac |
| | | agcggaacuucuacgagccccagaucaucaccaccgacaa |
| | | caccuucgugucuggcaacugcgacgucgugaucggcauu |
| | | gugaacaauaccguguacgacccucugcagcccgagcugg |
| | | acagcuucaaagaggaacuggacaaguacuuuaagaacca |
| | | cacaagccccgacguggaccugggcgauaucagcggaauc |
| | | aaugccagcgucgugaacauccagaaagagaucgaccggc |
| | | ugaacgagguggccaagaaucugaacgagagccugaucga |
| | | ccugcaagaacuggggaaguacgagcaguacaucaagugg |
| | | cccugguacaucuggcugggcuuuaucgccggacugauug |
| | | ccaucgugauggucacaaucaugcuguguugcaugaccag |
| | | cugcuguagcugccugaagggcuguuguagcuguggcagc |
| | | ugcugcaaguucgacgaggacgauucugagcccgugcuga |
| | | agggcgugaaacugcacuacacaugaugacucgagcuggu |
| | | acugcaugcacgcaaugcuagcugccccuuucccguccug |
| | | gguaccccgagucucccccgaccucgggucccagguaugc |
| | | ucccaccuccaccugccccacucaccaccucugcuaguuc |
| | | cagacaccucccaagcacgcagcaaugcagcucaaaacgc |
| | | uuagccuagccacacccccacgggaaacagcagugauuaa |
| | | ccuuuagcaauaaacgaaaguuuaacuaagcuauacuaac |
| | | cccaggguuggucaauuucgugccagccacacccuggagc |
| | | uagcaaaaaaaaaaaaaaaaaaaaaaaaaaaaaagcauau |
| | | gacuaaaaaaaaaaaaaaaaaaaaaaaaaaaaaaaaaaaa |
| | | aaaaaaaaaaaaaaaaaaaaaaaaaaaaaaaaa |
| 153 | Full length DNA construct encoding a | agaataaactagtattcttctggtccccacagactcagag |
| | SARS-CoV-2 S protein from an | agaacccgccaccatgttcgtgttcctggtgctgctgcct |
| | Omicron BA.2.75 variant | ctggtgtccagccagtgtgtgaacctgatcaccagaacac |
| | | agtcatacaccaacagctttaccagaggcgtgtactaccc |
| | | cgacaaggtgttcagatccagcgtgctgcactctacccag |
| | | gacctgttcctgcctttcttcagcaacgtgacctggttcc |
| | | acgccatccacgtgtccggcaccaatggcaccaagagatt |
| | | cgacaaccccgtgctgcccttcaacgacggggtgtacttt |
| | | gccagcaccgagaagtccaacatcatcagaggctggatct |
| | | tcggcaccacactggacagcaagacccagagcctgctgat |
| | | cgtgaacaacgccaccaacgtggtcatcaaagtgtgcgag |
| | | ttccagttctgcaacgaccccttcctggacgtctactacc |
| | | acgagaacaacaagagcaggatggaaagcgagctccgggt |
| | | gtacagcagcgccaacaactgcaccttcgagtacgtgtcc |
| | | cagcctttcctgatggacctggaaggcaagcagggcaact |
| | | tcaagaacctgcgcgagttcgtgtttaagaacatcgacgg |
| | | ctacttcaagatctacagcaagcacacccctgtgaacctc |
| | | ggccgggatctgcctcagggcttctctgctctggaacccc |
| | | tggtggatctgcccatcggcatcaacatcacccggtttca |
| | | gacactgctggccctgcacagaagctacctgacacctggc |
| | | gatagcagcagcagctggacagctggtgccgccgcttact |
| | | atgtgggctacctgcagcctagaaccttcctgctgaagta |
| | | caacgagaacggcaccatcaccgacgccgtggattgtgct |
| | | ctggatcctctgagcgagacaaagtgcaccctgaagtcct |
| | | tcaccgtggaaaagggcatctaccagaccagcaacttccg |
| | | ggtgcagcccaccgaatccatcgtgcggttccccaatatc |
| | | accaatctgtgccccttccacgaggtgttcaatgccacca |
| | | gattcgcctctgtgtacgcctggaaccggaagcggatcag |
| | | caattgcgtggccgactactccgtgctgtacaacttcgcc |
| | | cccttcttcgcattcaagtgctacggcgtgtcccctacca |
| | | agctgaacgacctgtgcttcacaaacgtgtacgccgacag |
| | | cttcgtgatccggggaaacgaagtgtcacagattgcccct |
| | | ggacagacaggcaacatcgccgactacaactacaagctgc |
| | | ccgacgacttcaccggctgtgtgattgcctggaacagcaa |
| | | caagctggactccaaagtcagcggcaactacaattacctg |
| | | taccggctgttccggaagtccaagctgaagcccttcgagc |
| | | gggacatctccaccgagatctatcaggccggcaacaagcc |
| | | ttgtaacggcgtggcaggcttcaactgctacttcccactg |
| | | cagtcctacggctttaggcccacatacggcgtgggccacc |
| | | agccctacagagtggtggtgctgagcttcgaactgctgca |
| | | tgcccctgccacagtgtgcggccctaagaaaagcaccaat |
| | | ctcgtgaagaacaaatgcgtgaacttcaacttcaacggcc |
| | | tgaccggcaccggcgtgctgacagagagcaacaagaagtt |
| | | cctgccattccagcagtttggccgggatatcgccgatacc |
| | | acagacgccgttagagatccccagacactggaaatcctgg |
| | | acatcaccccttgcagcttcggcggagtgtctgtgatcac |
| | | ccctggcaccaacaccagcaatcaggtggcagtgctgtac |
| | | cagggcgtgaactgtaccgaagtgcccgtggccattcacg |
| | | ccgatcagctgacacctacatggcgggtgtactccaccgg |
| | | cagcaatgtgtttcagaccagagccggctgtctgatcgga |
| | | gccgagtacgtgaacaatagctacgagtgcgacatcccca |
| | | tcggcgctggaatctgcgccagctaccagacacagacaaa |
| | | gagccaccggagagccagaagcgtggccagccagagcatc |
| | | attgcctacacaatgtctctgggcgccgagaacagcgtgg |
| | | cctactccaacaactctatcgctatccccaccaacttcac |
| | | catcagcgtgaccacagagatcctgcctgtgtccatgacc |
| | | aagaccagcgtggactgcaccatgtacatctgcggcgatt |
| | | ccaccgagtgctccaacctgctgctgcagtacggcagctt |
| | | ctgcacccagctgaaaagagccctgacagggatcgccgtg |
| | | gaacaggacaagaacacccaagaggtgttcgcccaagtga |
| | | agcagatctacaagacccctcctatcaagtacttcggcgg |
| | | cttcaatttcagccagattctgcccgatcctagcaagccc |
| | | agcaagcggagcttcatcgaggacctgctgttcaacaaag |
| | | tgacactggccgacgccggcttcatcaagcagtatggcga |
| | | ttgtctgggcgacattgccgccagggatctgatttgcgcc |
| | | cagaagtttaacggactgacagtgctgcctcctctgctga |
| | | ccgatgagatgatcgcccagtacacatctgccctgctggc |
| | | cggcacaatcacaagcggctggacatttggagcaggcgcc |
| | | gctctgcagatcccctttgctatgcagatggcctaccggt |
| | | tcaacggcatcggagtgacccagaatgtgctgtacgagaa |
| | | ccagaagctgatcgccaaccagttcaacagcgccatcggc |
| | | aagatccaggacagcctgagcagcacagcaagcgccctgg |
| | | gaaagctgcaggacgtggtcaaccacaatgcccaggcact |
| | | gaacaccctggtcaagcagctgtcctccaagttcggcgcc |
| | | atcagctctgtgctgaacgatatcctgagcagactggacc |
| | | ctcctgaggccgaggtgcagatcgacagactgatcacagg |
| | | cagactgcagagcctccagacatacgtgacccagcagctg |
| | | atcagagccgccgagattagagcctctgccaatctggccg |
| | | ccaccaagatgtctgagtgtgtgctgggccagagcaagag |
| | | agtggacttttgcggcaagggctaccacctgatgagcttc |
| | | cctcagtctgcccctcacggcgtggtgtttctgcacgtga |
| | | catatgtgcccgctcaagagaagaatttcaccaccgctcc |
| | | agccatctgccacgacggcaaagcccactttcctagagaa |
| | | ggcgtgttcgtgtccaacggcacccattggttcgtgacac |
| | | agcggaacttctacgagccccagatcatcaccaccgacaa |
| | | caccttcgtgtctggcaactgcgacgtcgtgatcggcatt |
| | | gtgaacaataccgtgtacgaccctctgcagcccgagctgg |
| | | acagcttcaaagaggaactggacaagtactttaagaacca |
| | | cacaagccccgacgtggacctgggcgatatcagcggaatc |
| | | aatgccagcgtcgtgaacatccagaaagagatcgaccggc |
| | | tgaacgaggtggccaagaatctgaacgagagcctgatcga |
| | | cctgcaagaactggggaagtacgagcagtacatcaagtgg |
| | | ccctggtacatctggctgggctttatcgccggactgattg |
| | | ccatcgtgatggtcacaatcatgctgtgttgcatgaccag |
| | | ctgctgtagctgcctgaagggctgttgtagctgtggcagc |
| | | tgctgcaagttcgacgaggacgattctgagcccgtgctga |
| | | agggcgtgaaactgcactacacatgatgactcgagctggt |
| | | actgcatgcacgcaatgctagctgcccctttcccgtcctg |
| | | ggtaccccgagtctcccccgacctcgggtcccaggtatgc |
| | | tcccacctccacctgccccactcaccacctctgctagttc |
| | | cagacacctcccaagcacgcagcaatgcagctcaaaacgc |
| | | ttagcctagccacacccccacgggaaacagcagtgattaa |
| | | cctttagcaataaacgaaagtttaactaagctatactaac |
| | | cccagggttggtcaatttcgtgccagccacaccctggagc |
| | | tagcaaaaaaaaaaaaaaaaaaaaaaaaaaaaaagcatat |
| | | gactaaaaaaaaaaaaaaaaaaaaaaaaaaaaaaaaaaaa |
| | | aaaaaaaaaaaaaaaaaaaaaaaaaaaaaaaaa |

TABLE 13
Sequence of one embodiment of an exemplary Omicron BA.2.75.2-specific RNA vaccine

| SEQ ID NO. | Brief Description | Sequence |
|---|---|---|
| 154 | Amino acid sequence | MFVFLVLLPLVSSQCVNLITRTQSYTNSFTRGVYYPDKVFRSSVLHSTQDLFLPFFSNVTWFHAIHVSGTNGTKR |
| | of RNA-encoded SARS- | FDNPVLPFNDGVYFASTEKSNIIRGWIFGTTLDSKTQSLLIVNNATNVVIKVCEFQFCNDPFLDVYYHENNKSRM |
| | COV-2 S protein from | ESELRVYSSANNCTFEYVSQPFLMDLEGKQGNFKNLREFVFKNIDGYFKIYSKHTPVNLGRDLPQGFSALEPLVD |
| | an Omicron BA.2.75.2 | LPIGINITRFQTLLALHRSYLTPGDSSSSWTAGAAAYYVGYLQPRTFLLKYNENGTITDAVDCALDPLSETKCTLK |
| | variant (with PRO | SFTVEKGIYQTSNFRVQPTESIVRFPNITNLCPFHEVFNATTFASVYAWNRKRISNCVADYSVLYNFAPFFAFKCY |
| | mutations at positions | GVSPTKLNDLCFTNVYADSFVIRGNEVSQIAPGQTGNIADYNYKLPDDFTGCVIAWNSNKLDSKVSGNYNYLYR |
| | corresponding to K986P | LFRKSKLKPFERDISTEIYQAGNKPCNGVAGSNCYFPLQSYGFRPTYGVGHQPYRVVVLSFELLHAPATVCGPKK |
| | and V987P of SEQ ID | STNLVKNKCVNFNFNGLTGTGVLTESNKKFLPFQQFGRDIADTTDAVRDPQTLEILDITPCSFGGVSVITPGTNT |
| | NO: 1; i.e., PRO | SNQVAVLYQGVNCTEVPVAIHADQLTPTWRVYSTGSNVFQTRAGCLIGAEYVNNSYECDIPIGAGICASYQTQT |
| | mutations at | KSHRRARSVASQSIIAYTMSLGAENSVAYSNNSIAIPTNFTISVTTEILPVSMTKTSVDCTMYICGDSTECSNLLL |
| | positions 983 and | QYGSFCTQLKRALTGIAVEQDKNTQEVFAQVKQIYKTPPIKYFGGFNFSQILPDPSKPSKRSFIEDLLFNKVTLAD |
| | 984 of SEQ ID NO: 85) | AGFIKQYGDCLGDIAARDLICAQKFNGLTVLPPLLTDEMIAQYTSALLAGTITSGWTFGAGAALQIPFAMQMAYR |
| | | FNGIGVTQNVLYENQKLIANQFNSAIGKIQDSLSSTASALGKLQDVVNHNAQALNTLVKQLSSKFGAISSVLNDI |
| | | LSRLDPPEAEVQIDRLITGRLQSLQTYVTQQLIRAAEIRASANLAATKMSECVLGQSKRVDFCGKGYHLMSFPQS |
| | | APHGVVFLHVTYVPAQEKNFTTAPAICHDGKAHFPREGVFVSNGTHWFVTQRNFYEPQIITTDNTFVSGNCDV |
| | | VIGIVNNTVYDPLQPELDSFKEELDKYFKNHTSPDVDLGDISGINASVVNIQKEIDRLNEVAKNLNESLINLQELG |
| | | KYEQYIKWPWYIWLGFIAGLIAIVMVTIMLCCMTSCCSCLKGCCSCGSCCKFDEDDSEPVLKGVKLHYT |
| 155 | RNA sequence encoding | auguucguguuccuggugcugcugccucugguguccagccagugugugaaccugaucaccagaacacagucauacaccaacagc |
| | a SARS-COV-2 S | uuuaccagaggcguguacuaccccgacaagguguucagauccagcgugcugcacucuacccaggaccuguuccugccuuucuuca |
| | protein from an | gcaacgugaccugguuccacgccauccacguguccggcaccaauggcaccaagagauucgacaaccccgugcugcccuucaacgac |
| | Omicron BA.2.75.2 | gggguguacuuugccagcaccgagaaguccaacaucaucagaggcuggaucuucggcaccacacuggacagcaagacccagagcc |
| | variant | ugcugaucgugaacaacgccaccaacguggucaucaaagugugcgaguuccaguucugcaacgaccccuuccuggacgucuacua |
| | | ccacgagaacaacaagagcaggauggaaagcgagcuccggguguacagcagcgccaacaacugcaccuucgaguacgugucccag |
| | | ccuuuccugauggaccuggaaggcaagcagggcaacuucaagaaccugcgcgaguucguguuuaagaacaucgacggcuacuuc |
| | | aagaucuacagcaagcacaccccugugaaccucggccgggaucugccucagggcuucucugcucuggaaccccugguggaucugc |
| | | ccaucggcaucaacaucacccgguuucagacacugcuggcccugcacagaagcuaccugacaccuggcgauagcagcagcagcugg |
| | | acagcuggugccgccgcuuacuaugugggcuaccugcagccuagaaccuuccugcugaaguacaacgagaacggcaccaucaccg |
| | | acgccguggauugugcucuggauccucugagcgagacaaagugcacccugaaguccuucaccguggaaaagggcaucuaccagac |
| | | cagcaacuuccgggugcagcccaccgaauccaucgugcgguuccccaauaucaccaaucugugccccuuccacgagguguucaaug |
| | | ccaccaccuucgccucuguguacgccuggaaccggaagcggaucagcaauugcguggccgacuacuccgugcuguacaacuucgc |
| | | ccccuucuucgcauucaagugcuacggcguguccccuaccaagcugaacgaccugugcuucacaaacguguacgccgacagcuuc |
| | | gugauccggggaaacgaagugucacagauugccccuggacagacaggcaacaucgccgacuacaacuacaagcugcccgacgacu |
| | | ucaccggcugugugauugccuggaacagcaacaagcuggacuccaaagucagcggcaacuacaauuaccuguaccggcuguuccg |
| | | gaaguccaagcugaagcccuucgagcgggacaucuccaccgagaucuaucaggccggcaacaagccuuguaacggcguggcaggc |
| | | agcaacugcuacuucccacugcaguccuacggcuuuaggcccacauacggcgugggccaccagcccuacagagugguggugcuga |
| | | gcuucgaacugcugcaugccccugccacagugugcggcccuaagaaaagcaccaaucucgugaagaacaaaugcgugaacuucaa |
| | | cuucaacggccugaccggcaccggcgugcugacagagagcaacaagaaguuccugccauuccagcaguuuggccgggauaucgcc |
| | | gauaccacagacgccguuagagauccccagacacuggaaauccuggacaucaccccuugcagcuucggcggagugucugugauca |
| | | ccccuggcaccaacaccagcaaucagguggcagugcuguaccagggcgugaacuguaccgaagugcccguggccauucacgccga |
| | | ucagcugacaccuacauggcggguguacuccaccggcagcaauguguuucagaccagagccggcugucugaucggagccgaguac |
| | | gugaacaauagcuacgagugcgacauccccaucggcgcuggaaucugcgccagcuaccagacacagacaaagagccaccggagag |
| | | ccagaagcguggccagccagagcaucauugccuacacaaugucucugggcgccgagaacagcguggccuacuccaacaacucuau |
| | | cgcuauccccaccaacuucaccaucagcgugaccacagagauccugccuguguccaugaccaagaccagcguggacugcaccaugu |
| | | acaucugcggcgauuccaccgagugcuccaaccugcugcugcaguacggcagcuucugcacccagcugaaaagagcccugacagg |
| | | gaucgccguggaacaggacaagaacacccaagagguguucgcccaagugaagcagaucuacaagaccccuccuaucaaguacuuc |
| | | ggcggcuucaauuucagccagauucugcccgauccuagcaagcccagcaagcggagcuucaucgaggaccugcuguucaacaaag |
| | | ugacacuggccgacgccggcuucaucaagcaguauggcgauugucugggcgacauugccgccagggaucugauuugcgcccaga |
| | | aguuuaacggacugacagugcugccuccucugcugaccgaugagaugaucgcccaguacacaucugcccugcuggccggcacaau |
| | | cacaagcggcuggacauuuggagcaggcgccgcucugcagauccccuuugcuaugcagauggccuaccgguucaacggcaucgga |
| | | gugacccagaaugugcuguacgagaaccagaagcugaucgccaaccaguucaacagcgccaucggcaagauccaggacagccuga |
| | | gcagcacagcaagcgcccugggaaagcugcaggacguggucaaccacaaugcccaggcacugaacacccuggucaagcagcugucc |
| | | uccaaguucggcgccaucagcucugugcugaacgauauccugagcagacuggacccuccugaggccgaggugcagaucgacagac |
| | | ugaucacaggcagacugcagagccuccagacauacgugacccagcagcugaucagagccgccgagauuagagccucugccaaucu |
| | | ggccgccaccaagaugucugagugugugcugggccagagcaagagaguggacuuuugcggcaagggcuaccaccugaugagcuu |
| | | cccucagucugccccucacggcgugguguuucugcacgugacauaugugcccgcucaagagaagaauuucaccaccgcuccagcc |
| | | aucugccacgacggcaaagcccacuuuccuagagaaggcguguucguguccaacggcacccauugguucgugacacagcggaacu |
| | | ucuacgagccccagaucaucaccaccgacaacaccuucgugucuggcaacugcgacgucgugaucggcauugugaacaauaccgu |
| | | guacgacccucugcagcccgagcuggacagcuucaaagaggaacuggacaaguacuuuaagaaccacacaagccccgacguggac |
| | | cugggcgauaucagcggaaucaaugccagcgucgugaacauccagaaagagaucgaccggcugaacgagguggccaagaaucug |
| | | aacgagagccugaucaaccugcaagaacuggggaaguacgagcaguacaucaaguggcccugguacaucuggcugggcuuuauc |
| | | gccggacugauugccaucgugauggucacaaucaugcuguguugcaugaccagcugcuguagcugccugaagggcuguuguagc |
| | | uguggcagcugcugcaaguucgacgaggacgauucugagcccgugcugaagggcgugaaacugcacuacacaugauga |
| 156 | DNA sequence encoding | atgttcgtgttcctggtgctgctgcctctggtgtccagccagtgtgtgaacctgatcaccagaacacagtcatacaccaacagctttaccagagg |
| | a SARS-COV-2 S | cgtgtactaccccgacaaggtgttcagatccagcgtgctgcactctacccaggacctgttcctgcctttcttcagcaacgtgacctggttccacgc |
| | protein from an | catccacgtgtccggcaccaatggcaccaagagattcgacaaccccgtgctgcccttcaacgacggggtgtactttgccagcaccgagaagt |
| | Omicron BA.2.75.2 | ccaacatcatcagaggctggatcttcggcaccacactggacagcaagacccagagcctgctgatcgtgaacaacgccaccaacgtggtcatc |
| | variant | aaagtgtgcgagttccagttctgcaacgaccccttcctggacgtctactaccacgagaacaacaagagcaggatggaaagcgagctccggg |
| | | tgtacagcagcgccaacaactgcaccttcgagtacgtgtcccagcctttcctgatggacctggaaggcaagcagggcaacttcaagaacctg |
| | | cgcgagttcgtgtttaagaacatcgacggctacttcaagatctacagcaagcacacccctgtgaacctcggccgggatctgcctcagggcttct |
| | | ctgctctggaacccctggtggatctgcccatcggcatcaacatcacccggtttcagacactgctggccctgcacagaagctacctgacacctgg |
| | | cgatagcagcagcagctggacagctggtgccgccgcttactatgtgggctacctgcagcctagaaccttcctgctgaagtacaacgagaacg |
| | | gcaccatcaccgacgccgtggattgtgctctggatcctctgagcgagacaaagtgcaccctgaagtccttcaccgtggaaaagggcatctacc |
| | | agaccagcaacttccgggtgcagcccaccgaatccatcgtgcggttccccaatatcaccaatctgtgccccttccacgaggtgttcaatgccac |
| | | caccttcgcctctgtgtacgcctggaaccggaagcggatcagcaattgcgtggccgactactccgtgctgtacaacttcgcccccttcttcgcatt |
| | | caagtgctacggcgtgtcccctaccaagctgaacgacctgtgcttcacaaacgtgtacgccgacagcttcgtgatccggggaaacgaagtgt |
| | | cacagattgcccctggacagacaggcaacatcgccgactacaactacaagctgcccgacgacttcaccggctgtgtgattgcctggaacagc |
| | | aacaagctggactccaaagtcagcggcaactacaattacctgtaccggctgttccggaagtccaagctgaagcccttcgagcgggacatctc |
| | | caccgagatctatcaggccggcaacaagccttgtaacggcgtggcaggcagcaactgctacttcccactgcagtcctacggctttaggcccac |
| | | atacggcgtgggccaccagccctacagagtggtggtgctgagcttcgaactgctgcatgcccctgccacagtgtgcggccctaagaaaagca |
| | | ccaatctcgtgaagaacaaatgcgtgaacttcaacttcaacggcctgaccggcaccggcgtgctgacagagagcaacaagaagttcctgcc |
| | | attccagcagtttggccgggatatcgccgataccacagacgccgttagagatccccagacactggaaatcctggacatcaccccttgcagctt |
| | | cggcggagtgtctgtgatcacccctggcaccaacaccagcaatcaggtggcagtgctgtaccagggcgtgaactgtaccgaagtgcccgtg |
| | | gccattcacgccgatcagctgacacctacatggcgggtgtactccaccggcagcaatgtgtttcagaccagagccggctgtctgatcggagcc |
| | | gagtacgtgaacaatagctacgagtgcgacatccccatcggcgctggaatctgcgccagctaccagacacagacaaagagccaccggaga |
| | | gccagaagcgtggccagccagagcatcattgcctacacaatgtctctgggcgccgagaacagcgtggcctactccaacaactctatcgctatc |
| | | cccaccaacttcaccatcagcgtgaccacagagatcctgcctgtgtccatgaccaagaccagcgtggactgcaccatgtacatctgcggcgat |
| | | tccaccgagtgctccaacctgctgctgcagtacggcagcttctgcacccagctgaaaagagccctgacagggatcgccgtggaacaggaca |
| | | agaacacccaagaggtgttcgcccaagtgaagcagatctacaagacccctcctatcaagtacttcggcggcttcaatttcagccagattctgcc |
| | | cgatcctagcaagcccagcaagcggagcttcatcgaggacctgctgttcaacaaagtgacactggccgacgccggcttcatcaagcagtatg |
| | | gcgattgtctgggcgacattgccgccagggatctgatttgcgcccagaagtttaacggactgacagtgctgcctcctctgctgaccgatgagat |
| | | gatcgcccagtacacatctgccctgctggccggcacaatcacaagcggctggacatttggagcaggcgccgctctgcagatcccctttgctat |
| | | gcagatggcctaccggttcaacggcatcggagtgacccagaatgtgctgtacgagaaccagaagctgatcgccaaccagttcaacagcgcc |
| | | atcggcaagatccaggacagcctgagcagcacagcaagcgccctgggaaagctgcaggacgtggtcaaccacaatgcccaggcactgaa |
| | | caccctggtcaagcagctgtcctccaagttcggcgccatcagctctgtgctgaacgatatcctgagcagactggaccctcctgaggccgaggt |
| | | gcagatcgacagactgatcacaggcagactgcagagcctccagacatacgtgacccagcagctgatcagagccgccgagattagagcctct |
| | | gccaatctggccgccaccaagatgtctgagtgtgtgctgggccagagcaagagagtggacttttgcggcaagggctaccacctgatgagctt |
| | | ccctcagtctgcccctcacggcgtggtgtttctgcacgtgacatatgtgcccgctcaagagaagaatttcaccaccgctccagccatctgccacg |
| | | acggcaaagcccactttcctagagaaggcgtgttcgtgtccaacggcacccattggttcgtgacacagcggaacttctacgagccccagatca |
| | | tcaccaccgacaacaccttcgtgtctggcaactgcgacgtcgtgatcggcattgtgaacaataccgtgtacgaccctctgcagcccgagctgg |
| | | acagcttcaaagaggaactggacaagtactttaagaaccacacaagccccgacgtggacctgggcgatatcagcggaatcaatgccagcgt |
| | | cgtgaacatccagaaagagatcgaccggctgaacgaggtggccaagaatctgaacgagagcctgatcaacctgcaagaactggggaagt |
| | | acgagcagtacatcaagtggccctggtacatctggctgggctttatcgccggactgattgccatcgtgatggtcacaatcatgctgtgttgcatg |
| | | accagctgctgtagctgcctgaagggctgttgtagctgtggcagctgctgcaagttcgacgaggacgattctgagcccgtgctgaagggcgt |
| | | gaaactgcactacacatgatga |
| 157 | Full length RNA | agaauaaacuaguauucuucugguccccacagacucagagagaacccgccaccauguucguguuccuggugcugcugccucugg |
| | construct encoding a | uguccagccagugugugaaccugaucaccagaacacagucauacaccaacagcuuuaccagaggcguguacuaccccgacaaggu |
| | SARS-COV-2 S protein | guucagauccagcgugcugcacucuacccaggaccuguuccugccuuucuucagcaacgugaccugguuccacgccauccacgug |
| | from an Omicron | uccggcaccaauggcaccaagagauucgacaaccccgugcugcccuucaacgacgggguguacuuugccagcaccgagaagucca |
| | BA.2.75.2 variant | acaucaucagaggcuggaucuucggcaccacacuggacagcaagacccagagccugcugaucgugaacaacgccaccaacguggu |
| | | caucaaagugugcgaguuccaguucugcaacgaccccuuccuggacgucuacuaccacgagaacaacaagagcaggauggaaagc |
| | | gagcuccggguguacagcagcgccaacaacugcaccuucgaguacgugucccagccuuuccugauggaccuggaaggcaagcagg |
| | | gcaacuucaagaaccugcgcgaguucguguuuaagaacaucgacggcuacuucaagaucuacagcaagcacaccccugugaaccu |
| | | cggccgggaucugccucagggcuucucugcucuggaaccccugguggaucugcccaucggcaucaacaucacccgguuucagaca |
| | | cugcuggcccugcacagaagcuaccugacaccuggcgauagcagcagcagcuggacagcuggugccgccgcuuacuaugugggcu |
| | | accugcagccuagaaccuuccugcugaaguacaacgagaacggcaccaucaccgacgccguggauugugcucuggauccucugag |
| | | cgagacaaagugcacccugaaguccuucaccguggaaaagggcaucuaccagaccagcaacuuccgggugcagcccaccgaaucca |
| | | ucgugcgguuccccaauaucaccaaucugugccccuuccacgagguguucaaugccaccaccuucgccucuguguacgccuggaa |
| | | ccggaagcggaucagcaauugcguggccgacuacuccgugcuguacaacuucgcccccuucuucgcauucaagugcuacggcgug |
| | | uccccuaccaagcugaacgaccugugcuucacaaacguguacgccgacagcuucgugauccggggaaacgaagugucacagauug |
| | | ccccuggacagacaggcaacaucgccgacuacaacuacaagcugcccgacgacuucaccggcugugugauugccuggaacagcaac |
| | | aagcuggacuccaaagucagcggcaacuacaauuaccuguaccggcuguuccggaaguccaagcugaagcccuucgagcgggaca |
| | | ucuccaccgagaucuaucaggccggcaacaagccuuguaacggcguggcaggcagcaacugcuacuucccacugcaguccuacgg |
| | | cuuuaggcccacauacggcgugggccaccagcccuacagagugguggugcugagcuucgaacugcugcaugccccugccacagug |
| | | ugcggcccuaagaaaagcaccaaucucgugaagaacaaaugcgugaacuucaacuucaacggccugaccggcaccggcgugcuga |
| | | cagagagcaacaagaaguuccugccauuccagcaguuuggccgggauaucgccgauaccacagacgccguuagagauccccagac |
| | | acuggaaauccuggacaucaccccuugcagcuucggcggagugucugugaucaccccuggcaccaacaccagcaaucagguggca |
| | | gugcuguaccagggcgugaacuguaccgaagugcccguggccauucacgccgaucagcugacaccuacauggcggguguacucca |
| | | ccggcagcaauguguuucagaccagagccggcugucugaucggagccgaguacgugaacaauagcuacgagugcgacauccccau |
| | | cggcgcuggaaucugcgccagcuaccagacacagacaaagagccaccggagagccagaagcguggccagccagagcaucauugcc |
| | | uacacaaugucucugggcgccgagaacagcguggccuacuccaacaacucuaucgcuauccccaccaacuucaccaucagcgugac |
| | | cacagagauccugccuguguccaugaccaagaccagcguggacugcaccauguacaucugcggcgauuccaccgagugcuccaac |
| | | agguguucgcccaagugaagcagaucuacaagaccccuccuaucaaguacuucggcggcuucaauuucagccagauucugcccga |
| | | uccuagcaagcccagcaagcggagcuucaucgaggaccugcuguucaacaaagugacacuggccgacgccggcuucaucaagcag |
| | | uauggcgauugucugggcgacauugccgccagggaucugauuugcgcccagaaguuuaacggacugacagugcugccuccucug |
| | | cugaccgaugagaugaucgcccaguacacaucugcccugcuggccggcacaaucacaagcggcuggacauuuggagcaggcgccg |
| | | cucugcagauccccuuugcuaugcagauggccuaccgguucaacggcaucggagugacccagaaugugcuguacgagaaccagaa |
| | | gcugaucgccaaccaguucaacagcgccaucggcaagauccaggacagccugagcagcacagcaagcgcccugggaaagcugcag |
| | | gacguggucaaccacaaugcccaggcacugaacacccuggucaagcagcuguccuccaaguucggcgccaucagcucugugcuga |
| | | acgauauccugagcagacuggacccuccugaggccgaggugcagaucgacagacugaucacaggcagacugcagagccuccagac |
| | | auacgugacccagcagcugaucagagccgccgagauuagagccucugccaaucuggccgccaccaagaugucugagugugugcug |
| | | ggccagagcaagagaguggacuuuugcggcaagggcuaccaccugaugagcuucccucagucugccccucacggcgugguguuu |
| | | cugcacgugacauaugugcccgcucaagagaagaauuucaccaccgcuccagccaucugccacgacggcaaagcccacuuuccuag |
| | | agaaggcguguucguguccaacggcacccauugguucgugacacagcggaacuucuacgagccccagaucaucaccaccgacaac |
| | | accuucgugucuggcaacugcgacgucgugaucggcauugugaacaauaccguguacgacccucugcagcccgagcuggacagcu |
| | | ucaaagaggaacuggacaaguacuuuaagaaccacacaagccccgacguggaccugggcgauaucagcggaaucaaugccagcgu |
| | | cgugaacauccagaaagagaucgaccggcugaacgagguggccaagaaucugaacgagagccugaucaaccugcaagaacuggg |
| | | gaaguacgagcaguacaucaaguggcccugguacaucuggcugggcuuuaucgccggacugauugccaucgugauggucacaau |
| | | caugcuguguugcaugaccagcugcuguagcugccugaagggcuguuguagcuguggcagcugcugcaaguucgacgaggacg |
| | | auucugagcccgugcugaagggcgugaaacugcacuacacaugaugacucgagcugguacugcaugcacgcaaugcuagcugcc |
| | | ccuuucccguccuggguaccccgagucucccccgaccucgggucccagguaugcucccaccuccaccugccccacucaccaccucug |
| | | cuaguuccagacaccucccaagcacgcagcaaugcagcucaaaacgcuuagccuagccacacccccacgggaaacagcagugauua |
| | | accuuuagcaauaaacgaaaguuuaacuaagcuauacuaaccccaggguuggucaauuucgugccagccacacccuggagcuagc |
| | | aaaaaaaaaaaaaaaaaaaaaaaaaaaaaagcauaugacuaaaaaaaaaaaaaaaaaaaaaaaaaaaaaaaaaaaaaaaaaaaa |
| | | aaaaaaaaaaaaaaaaaaaaaaaaa |
| 158 | Full length DNA | agaataaactagtattcttctggtccccacagactcagagagaacccgccaccatgttcgtgttcctggtgctgctgcctctggtgtccagccag |
| | construct encoding | tgtgtgaacctgatcaccagaacacagtcatacaccaacagctttaccagaggcgtgtactaccccgacaaggtgttcagatccagcgtgctg |
| | a SARS-COV-2 S | cactctacccaggacctgttcctgcctttcttcagcaacgtgacctggttccacgccatccacgtgtccggcaccaatggcaccaagagattcga |
| | protein from an | caaccccgtgctgcccttcaacgacggggtgtactttgccagcaccgagaagtccaacatcatcagaggctggatcttcggcaccacactgga |
| | Omicron BA.2.75.2 | cagcaagacccagagcctgctgatcgtgaacaacgccaccaacgtggtcatcaaagtgtgcgagttccagttctgcaacgaccccttcctgga |
| | variant | cgtctactaccacgagaacaacaagagcaggatggaaagcgagctccgggtgtacagcagcgccaacaactgcaccttcgagtacgtgtcc |
| | | cagcctttcctgatggacctggaaggcaagcagggcaacttcaagaacctgcgcgagttcgtgtttaagaacatcgacggctacttcaagatc |
| | | tacagcaagcacacccctgtgaacctcggccgggatctgcctcagggcttctctgctctggaacccctggtggatctgcccatcggcatcaaca |
| | | tcacccggtttcagacactgctggccctgcacagaagctacctgacacctggcgatagcagcagcagctggacagctggtgccgccgcttact |
| | | atgtgggctacctgcagcctagaaccttcctgctgaagtacaacgagaacggcaccatcaccgacgccgtggattgtgctctggatcctctga |
| | | gcgagacaaagtgcaccctgaagtccttcaccgtggaaaagggcatctaccagaccagcaacttccgggtgcagcccaccgaatccatcgt |
| | | gcggttccccaatatcaccaatctgtgccccttccacgaggtgttcaatgccaccaccttcgcctctgtgtacgcctggaaccggaagcggatca |
| | | gcaattgcgtggccgactactccgtgctgtacaacttcgcccccttcttcgcattcaagtgctacggcgtgtcccctaccaagctgaacgacctgt |
| | | gcttcacaaacgtgtacgccgacagcttcgtgatccggggaaacgaagtgtcacagattgcccctggacagacaggcaacatcgccgacta |
| | | caactacaagctgcccgacgacttcaccggctgtgtgattgcctggaacagcaacaagctggactccaaagtcagcggcaactacaattacct |
| | | gtaccggctgttccggaagtccaagctgaagcccttcgagcgggacatctccaccgagatctatcaggccggcaacaagccttgtaacggcg |
| | | tggcaggcagcaactgctacttcccactgcagtcctacggctttaggcccacatacggcgtgggccaccagccctacagagtggtggtgctga |
| | | gcttcgaactgctgcatgcccctgccacagtgtgcggccctaagaaaagcaccaatctcgtgaagaacaaatgcgtgaacttcaacttcaacg |
| | | gcctgaccggcaccggcgtgctgacagagagcaacaagaagttcctgccattccagcagtttggccgggatatcgccgataccacagacgc |
| | | cgttagagatccccagacactggaaatcctggacatcaccccttgcagcttcggcggagtgtctgtgatcacccctggcaccaacaccagcaa |
| | | tcaggtggcagtgctgtaccagggcgtgaactgtaccgaagtgcccgtggccattcacgccgatcagctgacacctacatggcgggtgtactc |
| | | caccggcagcaatgtgtttcagaccagagccggctgtctgatcggagccgagtacgtgaacaatagctacgagtgcgacatccccatcggcg |
| | | ctggaatctgcgccagctaccagacacagacaaagagccaccggagagccagaagcgtggccagccagagcatcattgcctacacaatgt |
| | | ctctgggcgccgagaacagcgtggcctactccaacaactctatcgctatccccaccaacttcaccatcagcgtgaccacagagatcctgcctgt |
| | | gtccatgaccaagaccagcgtggactgcaccatgtacatctgcggcgattccaccgagtgctccaacctgctgctgcagtacggcagcttctg |
| | | cacccagctgaaaagagccctgacagggatcgccgtggaacaggacaagaacacccaagaggtgttcgcccaagtgaagcagatctaca |
| | | agacccctcctatcaagtacttcggcggcttcaatttcagccagattctgcccgatcctagcaagcccagcaagcggagcttcatcgaggacct |
| | | gctgttcaacaaagtgacactggccgacgccggcttcatcaagcagtatggcgattgtctgggcgacattgccgccagggatctgatttgcgc |
| | | ccagaagtttaacggactgacagtgctgcctcctctgctgaccgatgagatgatcgcccagtacacatctgccctgctggccggcacaatcac |
| | | aagcggctggacatttggagcaggcgccgctctgcagatcccctttgctatgcagatggcctaccggttcaacggcatcggagtgacccaga |
| | | atgtgctgtacgagaaccagaagctgatcgccaaccagttcaacagcgccatcggcaagatccaggacagcctgagcagcacagcaagcg |
| | | ccctgggaaagctgcaggacgtggtcaaccacaatgcccaggcactgaacaccctggtcaagcagctgtcctccaagttcggcgccatcagc |
| | | tctgtgctgaacgatatcctgagcagactggaccctcctgaggccgaggtgcagatcgacagactgatcacaggcagactgcagagcctcca |
| | | gacatacgtgacccagcagctgatcagagccgccgagattagagcctctgccaatctggccgccaccaagatgtctgagtgtgtgctgggcc |
| | | agagcaagagagtggacttttgcggcaagggctaccacctgatgagcttccctcagtctgcccctcacggcgtggtgtttctgcacgtgacata |
| | | tgtgcccgctcaagagaagaatttcaccaccgctccagccatctgccacgacggcaaagcccactttcctagagaaggcgtgttcgtgtccaa |
| | | cggcacccattggttcgtgacacagcggaacttctacgagccccagatcatcaccaccgacaacaccttcgtgtctggcaactgcgacgtcgt |
| | | gatcggcattgtgaacaataccgtgtacgaccctctgcagcccgagctggacagcttcaaagaggaactggacaagtactttaagaaccaca |
| | | caagccccgacgtggacctgggcgatatcagcggaatcaatgccagcgtcgtgaacatccagaaagagatcgaccggctgaacgaggtgg |
| | | ccaagaatctgaacgagagcctgatcaacctgcaagaactggggaagtacgagcagtacatcaagtggccctggtacatctggctgggcttt |
| | | atcgccggactgattgccatcgtgatggtcacaatcatgctgtgttgcatgaccagctgctgtagctgcctgaagggctgttgtagctgtggca |
| | | gctgctgcaagttcgacgaggacgattctgagcccgtgctgaagggcgtgaaactgcactacacatgatgactcgagctggtactgcatgca |
| | | cgcaatgctagctgcccctttcccgtcctgggtaccccgagtctcccccgacctcgggtcccaggtatgctcccacctccacctgccccactcacc |
| | | acctctgctagttccagacacctcccaagcacgcagcaatgcagctcaaaacgcttagcctagccacacccccacgggaaacagcagtgatt |
| | | aacctttagcaataaacgaaagtttaactaagctatactaaccccagggttggtcaatttcgtgccagccacaccctggagctagcaaaaaaa |

TABLE 14
Sequence of one embodiment of an exemplary Omicron BA.4.6/BF.7-specific RNA vaccine

| SEQ ID NO. | Brief Description | Sequence |
|---|---|---|
| 159 | Amino acid sequence of RNA-encoded | MFVFLVLLPLVSSQCVNLITRTQSYTNSFTRGVYYPDKVFRSSVLHSTQDLFLPFFSNVTWFHAISGTNGTKRFD |
| | SARS-COV-2 S protein from an | NPVLPFNDGVYFASTEKSNIIRGWIFGTTLDSKTQSLLIVNNATNVVIKVCEFQFCNDPFLDVYYHKNNKSWME |
| | Omicron BA.4.6/BF.7 variant (with | SEFRVYSSANNCTFEYVSQPFLMDLEGKQGNFKNLREFVFKNIDGYFKIYSKHTPINLGRDLPQGFSALEPLVDLP |
| | PRO mutations at positions | IGINITRFQTLLALHRSYLTPGDSSSGWTAGAAAYYVGYLQPRTFLLKYNENGTITDAVDCALDPLSETKCTLKSF |
| | corresponding to K986P and V987P of | TVEKGIYQTSNFRVQPTESIVRFPNITNLCPFDEVFNATTFASVYAWNRKRISNCVADYSVLYNFAPFFAFKCYG |
| | SEQ ID NO: 1; i.e., PRO mutations at | VSPTKLNDLCFTNVYADSFVIRGNEVRQIAPGQTGNIADYNYKLPDDFTGCVIAWNSNKLDSKVGGNYNYRYRL |
| | positions 981 and 982 of SEQ ID NO: | FRKSNLKPFERDISTEIYQAGNKPCNGVAGVNCYFPLQSYGFRPTYGVGHQPYRVVVLSFELLHAPATVCGPKKS |
| | 90) | TNLVKNKCVNFNFNGLTGTGVLTESNKKFLPFQQFGRDIADTTDAVRDPQTLEILDITPCSFGGVSVITPGTNTS |
| | | NQVAVLYQGVNCTEVPVAIHADQLTPTWRVYSTGSNVFQTRAGCLIGAEYVNNSYECDIPIGAGICASYQTQTK |
| | | SHRRARSVASQSIIAYTMSLGAENSVAYSNNSIAIPTNFTISVTTEILPVSMTKTSVDCTMYICGDSTECSNLLLQ |
| | | YGSFCTQLKRALTGIAVEQDKNTQEVFAQVKQIYKTPPIKYFGGFNFSQILPDPSKPSKRSFIEDLLFNKVTLADA |
| | | GFIKQYGDCLGDIAARDLICAQKFNGLTVLPPLLTDEMIAQYTSALLAGTITSGWTFGAGAALQIPFAMQMAYRF |
| | | NGIGVTQNVLYENQKLIANQFNSAIGKIQDSLSSTASALGKLQDVVNHNAQALNTLVKQLSSKFGAISSVLNDIL |
| | | SRLDPPEAEVQIDRLITGRLQSLQTYVTQQLIRAAEIRASANLAATKMSECVLGQSKRVDFCGKGYHLMSFPQSA |
| | | PHGVVFLHVTYVPAQEKNFTTAPAICHDGKAHFPREGVFVSNGTHWFVTQRNFYEPQIITTDNTFVSGNCDVVI |
| | | GIVNNTVYDPLQPELDSFKEELDKYFKNHTSPDVDLGDISGINASVVNIQKEIDRLNEVAKNLNESLIDLQELGKY |
| | | EQYIKWPWYIWLGFIAGLIAIVMVTIMLCCMTSCCSCLKGCCSCGSCCKFDEDDSEPVLKGVKLHYT |
| 160 | RNA sequence encoding a SARS-COV-2 | auguucguguuccuggugcugcugccucugguguccagccagugugugaaccugaucaccagaacacagucauacaccaacagc |
| | S protein from an Omicron BA.4.6/BF.7 | uuuaccagaggcguguacuaccccgacaagguguucagauccagcgugcugcacucuacccaggaccuguuccugccuuucuuca |
| | variant | gcaacgugaccugguuccacgccaucuccggcaccaauggcaccaagagauucgacaaccccgugcugcccuucaacgacggggug |
| | | uacuuugccagcaccgagaaguccaacaucaucagaggcuggaucuucggcaccacacuggacagcaagacccagagccugcuga |
| | | ucgugaacaacgccaccaacguggucaucaaagugugcgaguuccaguucugcaacgaccccuuccuggacgucuacuaccacaa |
| | | gaacaacaagagcuggauggaaagcgaguuccggguguacagcagcgccaacaacugcaccuucgaguacgugucccagccuuuc |
| | | cugauggaccuggaaggcaagcagggcaacuucaagaaccugcgcgaguucguguuuaagaacaucgacggcuacuucaagauc |
| | | uacagcaagcacaccccuaucaaccucggccgggaucugccucagggcuucucugcucuggaaccccugguggaucugcccaucgg |
| | | caucaacaucacccgguuucagacacugcuggcccugcacagaagcuaccugacaccuggcgauagcagcagcggauggacagcu |
| | | ggugccgccgcuuacuaugugggcuaccugcagccuagaaccuuccugcugaaguacaacgagaacggcaccaucaccgacgccg |
| | | uggauugugcucuggauccucugagcgagacaaagugcacccugaaguccuucaccguggaaaagggcaucuaccagaccagca |
| | | acuuccgggugcagcccaccgaauccaucgugcgguuccccaauaucaccaaucugugccccuucgacgagguguucaaugccacc |
| | | accuucgccucuguguacgccuggaaccggaagcggaucagcaauugcguggccgacuacuccgugcuguacaacuucgcccccu |
| | | ucuucgcauucaagugcuacggcguguccccuaccaagcugaacgaccugugcuucacaaacguguacgccgacagcuucgugau |
| | | ccggggaaacgaagugcggcagauugccccuggacagacaggcaacaucgccgacuacaacuacaagcugcccgacgacuucaccg |
| | | gcugugugauugccuggaacagcaacaagcuggacuccaaagucggcggcaacuacaauuacagguaccggcuguuccggaagu |
| | | ccaaucugaagcccuucgagcgggacaucuccaccgagaucuaucaggccggcaacaagccuuguaacggcguggcaggcgugaa |
| | | cugcuacuucccacugcaguccuacggcuuuaggcccacauacggcgugggccaccagcccuacagagugguggugcugagcuuc |
| | | gaacugcugcaugccccugccacagugugcggcccuaagaaaagcaccaaucucgugaagaacaaaugcgugaacuucaacuuca |
| | | acggccugaccggcaccggcgugcugacagagagcaacaagaaguuccugccauuccagcaguuuggccgggauaucgccgauac |
| | | cacagacgccguuagagauccccagacacuggaaauccuggacaucaccccuugcagcuucggcggagugucugugaucaccccu |
| | | ggcaccaacaccagcaaucagguggcagugcuguaccagggcgugaacuguaccgaagugcccguggccauucacgccgaucagc |
| | | ugacaccuacauggcggguguacuccaccggcagcaauguguuucagaccagagccggcugucugaucggagccgaguacguga |
| | | acaauagcuacgagugcgacauccccaucggcgcuggaaucugcgccagcuaccagacacagacaaagagccaccggagagccaga |
| | | agcguggccagccagagcaucauugccuacacaaugucucugggcgccgagaacagcguggccuacuccaacaacucuaucgcua |
| | | uccccaccaacuucaccaucagcgugaccacagagauccugccuguguccaugaccaagaccagcguggacugcaccauguacauc |
| | | ugcggcgauuccaccgagugcuccaaccugcugcugcaguacggcagcuucugcacccagcugaaaagagcccugacagggaucg |
| | | ccguggaacaggacaagaacacccaagagguguucgcccaagugaagcagaucuacaagaccccuccuaucaaguacuucggcgg |
| | | cuucaauuucagccagauucugcccgauccuagcaagcccagcaagcggagcuucaucgaggaccugcuguucaacaaagugaca |
| | | cuggccgacgccggcuucaucaagcaguauggcgauugucugggcgacauugccgccagggaucugauuugcgcccagaaguuu |
| | | aacggacugacagugcugccuccucugcugaccgaugagaugaucgcccaguacacaucugcccugcuggccggcacaaucacaa |
| | | gcggcuggacauuuggagcaggcgccgcucugcagauccccuuugcuaugcagauggccuaccgguucaacggcaucggaguga |
| | | cccagaaugugcuguacgagaaccagaagcugaucgccaaccaguucaacagcgccaucggcaagauccaggacagccugagcag |
| | | cacagcaagcgcccugggaaagcugcaggacguggucaaccacaaugcccaggcacugaacacccuggucaagcagcuguccucca |
| | | aguucggcgccaucagcucugugcugaacgauauccugagcagacuggacccuccugaggccgaggugcagaucgacagacuga |
| | | ucacaggcagacugcagagccuccagacauacgugacccagcagcugaucagagccgccgagauuagagccucugccaaucuggc |
| | | cgccaccaagaugucugagugugugcugggccagagcaagagaguggacuuuugcggcaagggcuaccaccugaugagcuuccc |
| | | ucagucugccccucacggcgugguguuucugcacgugacauaugugcccgcucaagagaagaauuucaccaccgcuccagccauc |
| | | ugccacgacggcaaagcccacuuuccuagagaaggcguguucguguccaacggcacccauugguucgugacacagcggaacuucu |
| | | acgagccccagaucaucaccaccgacaacaccuucgugucuggcaacugcgacgucgugaucggcauugugaacaauaccgugua |
| | | cgacccucugcagcccgagcuggacagcuucaaagaggaacuggacaaguacuuuaagaaccacacaagccccgacguggaccug |
| | | ggcgauaucagcggaaucaaugccagcgucgugaacauccagaaagagaucgaccggcugaacgagguggccaagaaucugaac |
| | | gagagccugaucgaccugcaagaacuggggaaguacgagcaguacaucaaguggcccugguacaucuggcugggcuuuaucgcc |
| | | ggacugauugccaucgugauggucacaaucaugcuguguugcaugaccagcugcuguagcugccugaagggcuguuguagcug |
| | | uggcagcugcugcaaguucgacgaggacgauucugagcccgugcugaagggcgugaaacugcacuacacaugauga |
| 161 | DNA sequence encoding a SARS-COV-2 | atgttcgtgttcctggtgctgctgcctctggtgtccagccagtgtgtgaacctgatcaccagaacacagtcatacaccaacagctttaccagagg |
| | S protein from an Omicron BA.4.6/BF.7 | cgtgtactaccccgacaaggtgttcagatccagcgtgctgcactctacccaggacctgttcctgcctttcttcagcaacgtgacctggttccacgc |
| | variant | catctccggcaccaatggcaccaagagattcgacaaccccgtgctgcccttcaacgacggggtgtactttgccagcaccgagaagtccaaca |
| | | tcatcagaggctggatcttcggcaccacactggacagcaagacccagagcctgctgatcgtgaacaacgccaccaacgtggtcatcaaagtg |
| | | tgcgagttccagttctgcaacgaccccttcctggacgtctactaccacaagaacaacaagagctggatggaaagcgagttccgggtgtacag |
| | | cagcgccaacaactgcaccttcgagtacgtgtcccagcctttcctgatggacctggaaggcaagcagggcaacttcaagaacctgcgcgagt |
| | | tcgtgtttaagaacatcgacggctacttcaagatctacagcaagcacacccctatcaacctcggccgggatctgcctcagggcttctctgctctg |
| | | gaacccctggtggatctgcccatcggcatcaacatcacccggtttcagacactgctggccctgcacagaagctacctgacacctggcgatagc |
| | | agcagcggatggacagctggtgccgccgcttactatgtgggctacctgcagcctagaaccttcctgctgaagtacaacgagaacggcaccat |
| | | caccgacgccgtggattgtgctctggatcctctgagcgagacaaagtgcaccctgaagtccttcaccgtggaaaagggcatctaccagacca |
| | | gcaacttccgggtgcagcccaccgaatccatcgtgcggttccccaatatcaccaatctgtgccccttcgacgaggtgttcaatgccaccaccttc |
| | | gcctctgtgtacgcctggaaccggaagcggatcagcaattgcgtggccgactactccgtgctgtacaacttcgcccccttcttcgcattcaagtg |
| | | ctacggcgtgtcccctaccaagctgaacgacctgtgcttcacaaacgtgtacgccgacagcttcgtgatccggggaaacgaagtgcggcaga |
| | | ttgcccctggacagacaggcaacatcgccgactacaactacaagctgcccgacgacttcaccggctgtgtgattgcctggaacagcaacaag |
| | | ctggactccaaagtcggcggcaactacaattacaggtaccggctgttccggaagtccaatctgaagcccttcgagcgggacatctccaccga |
| | | gatctatcaggccggcaacaagccttgtaacggcgtggcaggcgtgaactgctacttcccactgcagtcctacggctttaggcccacatacgg |
| | | cgtgggccaccagccctacagagtggtggtgctgagcttcgaactgctgcatgcccctgccacagtgtgcggccctaagaaaagcaccaatc |
| | | tcgtgaagaacaaatgcgtgaacttcaacttcaacggcctgaccggcaccggcgtgctgacagagagcaacaagaagttcctgccattccag |
| | | cagtttggccgggatatcgccgataccacagacgccgttagagatccccagacactggaaatcctggacatcaccccttgcagcttcggcgg |
| | | agtgtctgtgatcacccctggcaccaacaccagcaatcaggtggcagtgctgtaccagggcgtgaactgtaccgaagtgcccgtggccattc |
| | | acgccgatcagctgacacctacatggcgggtgtactccaccggcagcaatgtgtttcagaccagagccggctgtctgatcggagccgagtac |
| | | gtgaacaatagctacgagtgcgacatccccatcggcgctggaatctgcgccagctaccagacacagacaaagagccaccggagagccaga |
| | | agcgtggccagccagagcatcattgcctacacaatgtctctgggcgccgagaacagcgtggcctactccaacaactctatcgctatccccacc |
| | | aacttcaccatcagcgtgaccacagagatcctgcctgtgtccatgaccaagaccagcgtggactgcaccatgtacatctgcggcgattccacc |
| | | gagtgctccaacctgctgctgcagtacggcagcttctgcacccagctgaaaagagccctgacagggatcgccgtggaacaggacaagaac |
| | | acccaagaggtgttcgcccaagtgaagcagatctacaagacccctcctatcaagtacttcggcggcttcaatttcagccagattctgcccgatc |
| | | ctagcaagcccagcaagcggagcttcatcgaggacctgctgttcaacaaagtgacactggccgacgccggcttcatcaagcagtatggcgat |
| | | tgtctgggcgacattgccgccagggatctgatttgcgcccagaagtttaacggactgacagtgctgcctcctctgctgaccgatgagatgatcg |
| | | cccagtacacatctgccctgctggccggcacaatcacaagcggctggacatttggagcaggcgccgctctgcagatcccctttgctatgcaga |
| | | tggcctaccggttcaacggcatcggagtgacccagaatgtgctgtacgagaaccagaagctgatcgccaaccagttcaacagcgccatcgg |
| | | caagatccaggacagcctgagcagcacagcaagcgccctgggaaagctgcaggacgtggtcaaccacaatgcccaggcactgaacaccct |
| | | ggtcaagcagctgtcctccaagttcggcgccatcagctctgtgctgaacgatatcctgagcagactggaccctcctgaggccgaggtgcagat |
| | | cgacagactgatcacaggcagactgcagagcctccagacatacgtgacccagcagctgatcagagccgccgagattagagcctctgccaat |
| | | ctggccgccaccaagatgtctgagtgtgtgctgggccagagcaagagagtggacttttgcggcaagggctaccacctgatgagcttccctca |
| | | gtctgcccctcacggcgtggtgtttctgcacgtgacatatgtgcccgctcaagagaagaatttcaccaccgctccagccatctgccacgacggc |
| | | aaagcccactttcctagagaaggcgtgttcgtgtccaacggcacccattggttcgtgacacagcggaacttctacgagccccagatcatcacc |
| | | accgacaacaccttcgtgtctggcaactgcgacgtcgtgatcggcattgtgaacaataccgtgtacgaccctctgcagcccgagctggacagc |
| | | ttcaaagaggaactggacaagtactttaagaaccacacaagccccgacgtggacctgggcgatatcagcggaatcaatgccagcgtcgtga |
| | | acatccagaaagagatcgaccggctgaacgaggtggccaagaatctgaacgagagcctgatcgacctgcaagaactggggaagtacgag |
| | | cagtacatcaagtggccctggtacatctggctgggctttatcgccggactgattgccatcgtgatggtcacaatcatgctgtgttgcatgaccag |
| | | ctgctgtagctgcctgaagggctgttgtagctgtggcagctgctgcaagttcgacgaggacgattctgagcccgtgctgaagggcgtgaaact |
| | | gcactacacatgatga |
| 162 | Full length RNA construct encoding a | agaauaaacuaguauucuucugguccccacagacucagagagaacccgccaccauguucguguuccuggugcugcugccucugg |
| | SARS-COV-2 S protein from an | uguccagccagugugugaaccugaucaccagaacacagucauacaccaacagcuuuaccagaggcguguacuaccccgacaaggu |
| | Omicron BA.4.6/BF.7 variant | guucagauccagcgugcugcacucuacccaggaccuguuccugccuuucuucagcaacgugaccugguuccacgccaucuccggc |
| | | accaauggcaccaagagauucgacaaccccgugcugcccuucaacgacgggguguacuuugccagcaccgagaaguccaacauca |
| | | ucagaggcuggaucuucggcaccacacuggacagcaagacccagagccugcugaucgugaacaacgccaccaacguggucaucaa |
| | | agugugcgaguuccaguucugcaacgaccccuuccuggacgucuacuaccacaagaacaacaagagcuggauggaaagcgaguuc |
| | | cggguguacagcagcgccaacaacugcaccuucgaguacgugucccagccuuuccugauggaccuggaaggcaagcagggcaacu |
| | | ucaagaaccugcgcgaguucguguuuaagaacaucgacggcuacuucaagaucuacagcaagcacaccccuaucaaccucggccg |
| | | ggaucugccucagggcuucucugcucuggaaccccugguggaucugcccaucggcaucaacaucacccgguuucagacacugcug |
| | | gcccugcacagaagcuaccugacaccuggcgauagcagcagcggauggacagcuggugccgccgcuuacuaugugggcuaccugc |
| | | agccuagaaccuuccugcugaaguacaacgagaacggcaccaucaccgacgccguggauugugcucuggauccucugagcgagac |
| | | aaagugcacccugaaguccuucaccguggaaaagggcaucuaccagaccagcaacuuccgggugcagcccaccgaauccaucgug |
| | | cgguuccccaauaucaccaaucugugccccuucgacgagguguucaaugccaccaccuucgccucuguguacgccuggaaccgga |
| | | agcggaucagcaauugcguggccgacuacuccgugcuguacaacuucgcccccuucuucgcauucaagugcuacggcgugucccc |
| | | uaccaagcugaacgaccugugcuucacaaacguguacgccgacagcuucgugauccggggaaacgaagugcggcagauugccccu |
| | | ggacagacaggcaacaucgccgacuacaacuacaagcugcccgacgacuucaccggcugugugauugccuggaacagcaacaagc |
| | | uggacuccaaagucggcggcaacuacaauuacagguaccggcuguuccggaaguccaaucugaagcccuucgagcgggacaucuc |
| | | caccgagaucuaucaggccggcaacaagccuuguaacggcguggcaggcgugaacugcuacuucccacugcaguccuacggcuuu |
| | | aggcccacauacggcgugggccaccagcccuacagagugguggugcugagcuucgaacugcugcaugccccugccacagugugcg |
| | | gcccuaagaaaagcaccaaucucgugaagaacaaaugcgugaacuucaacuucaacggccugaccggcaccggcgugcugacaga |
| | | gagcaacaagaaguuccugccauuccagcaguuuggccgggauaucgccgauaccacagacgccguuagagauccccagacacug |
| | | gaaauccuggacaucaccccuugcagcuucggcggagugucugugaucaccccuggcaccaacaccagcaaucagguggcagugc |
| | | uguaccagggcgugaacuguaccgaagugcccguggccauucacgccgaucagcugacaccuacauggcggguguacuccaccgg |
| | | cagcaauguguuucagaccagagccggcugucugaucggagccgaguacgugaacaauagcuacgagugcgacauccccaucggc |
| | | gcuggaaucugcgccagcuaccagacacagacaaagagccaccggagagccagaagcguggccagccagagcaucauugccuacac |
| | | aaugucucugggcgccgagaacagcguggccuacuccaacaacucuaucgcuauccccaccaacuucaccaucagcgugaccacag |
| | | agauccugccuguguccaugaccaagaccagcguggacugcaccauguacaucugcggcgauuccaccgagugcuccaaccugcu |
| | | gcugcaguacggcagcuucugcacccagcugaaaagagcccugacagggaucgccguggaacaggacaagaacacccaagaggug |
| | | uucgcccaagugaagcagaucuacaagaccccuccuaucaaguacuucggcggcuucaauuucagccagauucugcccgauccua |
| | | gcaagcccagcaagcggagcuucaucgaggaccugcuguucaacaaagugacacuggccgacgccggcuucaucaagcaguaugg |
| | | cgauugucugggcgacauugccgccagggaucugauuugcgcccagaaguuuaacggacugacagugcugccuccucugcugac |
| | | cgaugagaugaucgcccaguacacaucugcccugcuggccggcacaaucacaagcggcuggacauuuggagcaggcgccgcucug |
| | | cagauccccuuugcuaugcagauggccuaccgguucaacggcaucggagugacccagaaugugcuguacgagaaccagaagcug |
| | | aucgccaaccaguucaacagcgccaucggcaagauccaggacagccugagcagcacagcaagcgcccugggaaagcugcaggacg |
| | | uggucaaccacaaugcccaggcacugaacacccuggucaagcagcuguccuccaaguucggcgccaucagcucugugcugaacga |
| | | uauccugagcagacuggacccuccugaggccgaggugcagaucgacagacugaucacaggcagacugcagagccuccagacauac |
| | | gugacccagcagcugaucagagccgccgagauuagagccucugccaaucuggccgccaccaagaugucugagugugugcugggcc |
| | | agagcaagagaguggacuuuugcggcaagggcuaccaccugaugagcuucccucagucugccccucacggcgugguguuucugc |
| | | acgugacauaugugcccgcucaagagaagaauuucaccaccgcuccagccaucugccacgacggcaaagcccacuuuccuagagaa |
| | | ggcguguucguguccaacggcacccauugguucgugacacagcggaacuucuacgagccccagaucaucaccaccgacaacaccu |
| | | ucgugucuggcaacugcgacgucgugaucggcauugugaacaauaccguguacgacccucugcagcccgagcuggacagcuucaa |
| | | agaggaacuggacaaguacuuuaagaaccacacaagccccgacguggaccugggcgauaucagcggaaucaaugccagcgucgu |
| | | gaacauccagaaagagaucgaccggcugaacgagguggccaagaaucugaacgagagccugaucgaccugcaagaacuggggaa |
| | | guacgagcaguacaucaaguggcccugguacaucuggcugggcuuuaucgccggacugauugccaucgugauggucacaaucau |
| | | gcuguguugcaugaccagcugcuguagcugccugaagggcuguuguagcuguggcagcugcugcaaguucgacgaggacgauu |
| | | cugagcccgugcugaagggcgugaaacugcacuacacaugaugacucgagcugguacugcaugcacgcaaugcuagcugccccuu |
| | | ucccguccuggguaccccgagucucccccgaccucgggucccagguaugcucccaccuccaccugccccacucaccaccucugcuag |
| | | uuccagacaccucccaagcacgcagcaaugcagcucaaaacgcuuagccuagccacacccccacgggaaacagcagugauuaaccu |
| | | uuagcaauaaacgaaaguuuaacuaagcuauacuaaccccaggguuggucaauuucgugccagccacacccuggagcuagcaaa |
| | | aaaaaaaaaaaaaaaaaaaaaaaaaaagcauaugacuaaaaaaaaaaaaaaaaaaaaaaaaaaaaaaaaaaaaaaaaaaaaaaa |
| | | aaaaaaaaaaaaaaaaaaaaaa |
| 163 | Full length DNA construct encoding a | agaataaactagtattcttctggtccccacagactcagagagaacccgccaccatgttcgtgttcctggtgctgctgcctctggtgtccagccag |
| | SARS-COV-2 S protein from an | tgtgtgaacctgatcaccagaacacagtcatacaccaacagctttaccagaggcgtgtactaccccgacaaggtgttcagatccagcgtgctg |
| | Omicron BA.4.6/BF.7 variant | cactctacccaggacctgttcctgcctttcttcagcaacgtgacctggttccacgccatctccggcaccaatggcaccaagagattcgacaaccc |
| | | cgtgctgcccttcaacgacggggtgtactttgccagcaccgagaagtccaacatcatcagaggctggatcttcggcaccacactggacagca |
| | | agacccagagcctgctgatcgtgaacaacgccaccaacgtggtcatcaaagtgtgcgagttccagttctgcaacgaccccttcctggacgtct |
| | | actaccacaagaacaacaagagctggatggaaagcgagttccgggtgtacagcagcgccaacaactgcaccttcgagtacgtgtcccagcc |
| | | tttcctgatggacctggaaggcaagcagggcaacttcaagaacctgcgcgagttcgtgtttaagaacatcgacggctacttcaagatctacag |
| | | caagcacacccctatcaacctcggccgggatctgcctcagggcttctctgctctggaacccctggtggatctgcccatcggcatcaacatcacc |
| | | cggtttcagacactgctggccctgcacagaagctacctgacacctggcgatagcagcagcggatggacagctggtgccgccgcttactatgt |
| | | gggctacctgcagcctagaaccttcctgctgaagtacaacgagaacggcaccatcaccgacgccgtggattgtgctctggatcctctgagcga |
| | | gacaaagtgcaccctgaagtccttcaccgtggaaaagggcatctaccagaccagcaacttccgggtgcagcccaccgaatccatcgtgcggt |
| | | tccccaatatcaccaatctgtgccccttcgacgaggtgttcaatgccaccaccttcgcctctgtgtacgcctggaaccggaagcggatcagcaa |
| | | ttgcgtggccgactactccgtgctgtacaacttcgcccccttcttcgcattcaagtgctacggcgtgtcccctaccaagctgaacgacctgtgctt |
| | | cacaaacgtgtacgccgacagcttcgtgatccggggaaacgaagtgcggcagattgcccctggacagacaggcaacatcgccgactacaa |
| | | ctacaagctgcccgacgacttcaccggctgtgtgattgcctggaacagcaacaagctggactccaaagtcggcggcaactacaattacaggt |
| | | accggctgttccggaagtccaatctgaagcccttcgagcgggacatctccaccgagatctatcaggccggcaacaagccttgtaacggcgtg |
| | | gcaggcgtgaactgctacttcccactgcagtcctacggctttaggcccacatacggcgtgggccaccagccctacagagtggtggtgctgagc |
| | | ttcgaactgctgcatgcccctgccacagtgtgcggccctaagaaaagcaccaatctcgtgaagaacaaatgcgtgaacttcaacttcaacggc |
| | | ctgaccggcaccggcgtgctgacagagagcaacaagaagttcctgccattccagcagtttggccgggatatcgccgataccacagacgccg |
| | | ttagagatccccagacactggaaatcctggacatcaccccttgcagcttcggcggagtgtctgtgatcacccctggcaccaacaccagcaatc |
| | | aggtggcagtgctgtaccagggcgtgaactgtaccgaagtgcccgtggccattcacgccgatcagctgacacctacatggcgggtgtactcc |
| | | accggcagcaatgtgtttcagaccagagccggctgtctgatcggagccgagtacgtgaacaatagctacgagtgcgacatccccatcggcgc |
| | | tggaatctgcgccagctaccagacacagacaaagagccaccggagagccagaagcgtggccagccagagcatcattgcctacacaatgtc |
| | | tctgggcgccgagaacagcgtggcctactccaacaactctatcgctatccccaccaacttcaccatcagcgtgaccacagagatcctgcctgtg |
| | | tccatgaccaagaccagcgtggactgcaccatgtacatctgcggcgattccaccgagtgctccaacctgctgctgcagtacggcagcttctgc |
| | | acccagctgaaaagagccctgacagggatcgccgtggaacaggacaagaacacccaagaggtgttcgcccaagtgaagcagatctacaa |
| | | gacccctcctatcaagtacttcggcggcttcaatttcagccagattctgcccgatcctagcaagcccagcaagcggagcttcatcgaggacctg |
| | | ctgttcaacaaagtgacactggccgacgccggcttcatcaagcagtatggcgattgtctgggcgacattgccgccagggatctgatttgcgcc |
| | | cagaagtttaacggactgacagtgctgcctcctctgctgaccgatgagatgatcgcccagtacacatctgccctgctggccggcacaatcaca |
| | | agcggctggacatttggagcaggcgccgctctgcagatcccctttgctatgcagatggcctaccggttcaacggcatcggagtgacccagaat |
| | | gtgctgtacgagaaccagaagctgatcgccaaccagttcaacagcgccatcggcaagatccaggacagcctgagcagcacagcaagcgcc |
| | | ctgggaaagctgcaggacgtggtcaaccacaatgcccaggcactgaacaccctggtcaagcagctgtcctccaagttcggcgccatcagctc |
| | | tgtgctgaacgatatcctgagcagactggaccctcctgaggccgaggtgcagatcgacagactgatcacaggcagactgcagagcctccag |
| | | acatacgtgacccagcagctgatcagagccgccgagattagagcctctgccaatctggccgccaccaagatgtctgagtgtgtgctgggcca |
| | | gagcaagagagtggacttttgcggcaagggctaccacctgatgagcttccctcagtctgcccctcacggcgtggtgtttctgcacgtgacatat |
| | | gtgcccgctcaagagaagaatttcaccaccgctccagccatctgccacgacggcaaagcccactttcctagagaaggcgtgttcgtgtccaac |
| | | ggcacccattggttcgtgacacagcggaacttctacgagccccagatcatcaccaccgacaacaccttcgtgtctggcaactgcgacgtcgtg |
| | | atcggcattgtgaacaataccgtgtacgaccctctgcagcccgagctggacagcttcaaagaggaactggacaagtactttaagaaccacac |
| | | aagccccgacgtggacctgggcgatatcagcggaatcaatgccagcgtcgtgaacatccagaaagagatcgaccggctgaacgaggtggc |
| | | caagaatctgaacgagagcctgatcgacctgcaagaactggggaagtacgagcagtacatcaagtggccctggtacatctggctgggcttta |
| | | tcgccggactgattgccatcgtgatggtcacaatcatgctgtgttgcatgaccagctgctgtagctgcctgaagggctgttgtagctgtggcagc |
| | | tgctgcaagttcgacgaggacgattctgagcccgtgctgaagggcgtgaaactgcactacacatgatgactcgagctggtactgcatgcacg |
| | | caatgctagctgcccctttcccgtcctgggtaccccgagtctcccccgacctcgggtcccaggtatgctcccacctccacctgccccactcaccac |
| | | ctctgctagttccagacacctcccaagcacgcagcaatgcagctcaaaacgcttagcctagccacacccccacgggaaacagcagtgattaa |
| | | cctttagcaataaacgaaagtttaactaagctatactaaccccagggttggtcaatttcgtgccagccacaccctggagctagcaaaaaaaaa |
| | | aaaaaaaaaaaaaaaaaaaaagcatatgactaaaaaaaaaaaaaaaaaaaaaaaaaaaaaaaaaaaaaaaaaaaaaaaaaaaaaa |
| | | aaaaaaaaaaaaaaa |

TABLE 15
Sequence of one embodiment of an exemplary Omicron XBB-specific RNA vaccine

| SEQ ID NO. | Brief Description | Sequence |
|---|---|---|
| 164 | Amino acid sequence | MFVFLVLLPLVSSQCVNLITRTQSYTNSFTRGVYYPDKVFRSSVLHSTQDLFLPFFSNVTWFHAIHVSGTNGTKR |
| | of RNA-encoded SARS- | FDNPALPFNDGVYFASTEKSNIIRGWIFGTTLDSKTQSLLIVNNATNVVIKVCEFQFCNDPFLDVYQKNNKSWM |
| | COV-2 S protein | ESEFRVYSSANNCTFEYVSQPFLMDLEGKEGNFKNLREFVFKNIDGYFKIYSKHTPINLERDLPQGFSALEPLVDL |
| | from an Omicron | PIGINITRFQTLLALHRSYLTPGDSSSGWTAGAAAYYVGYLQPRTFLLKYNENGTITDAVDCALDPLSETKCTLKS |
| | XBB variant (with | FTVEKGIYQTSNFRVQPTESIVRFPNITNLCPFHEVFNATTFASVYAWNRKRISNCVADYSVIYNFAPFFAFKCYG |
| | PRO mutations at | VSPTKLNDLCFTNVYADSFVIRGNEVSQIAPGQTGNIADYNYKLPDDFTGCVIAWNSNKLDSKPSGNYNYLYRL |
| | positions | FRKSKLKPFERDISTEIYQAGNKPCNGVAGSNCYSPLQSYGFRPTYGVGHQPYRVVVLSFELLHAPATVCGPKKS |
| | corresponding to | TNLVKNKCVNFNFNGLTGTGVLTESNKKFLPFQQFGRDIADTTDAVRDPQTLEILDITPCSFGGVSVITPGTNTS |
| | K986P and V987P of | NQVAVLYQGVNCTEVPVAIHADQLTPTWRVYSTGSNVFQTRAGCLIGAEYVNNSYECDIPIGAGICASYQTQTK |
| | SEQ ID NO: 1; i.e., | SHRRARSVASQSIIAYTMSLGAENSVAYSNNSIAIPTNFTISVTTEILPVSMTKTSVDCTMYICGDSTECSNLLLQ |
| | PRO mutations at | YGSFCTQLKRALTGIAVEQDKNTQEVFAQVKQIYKTPPIKYFGGFNFSQILPDPSKPSKRSFIEDLLFNKVTLADA |
| | positions 981 and | GFIKQYGDCLGDIAARDLICAQKFNGLTVLPPLLTDEMIAQYTSALLAGTITSGWTFGAGAALQIPFAMQMAYRF |
| | 982 of SEQ ID | NGIGVTQNVLYENQKLIANQFNSAIGKIQDSLSSTASALGKLQDVVNHNAQALNTLVKQLSSKFGAISSVLNDIL |
| | NO: 95) | SRLDPPEAEVQIDRLITGRLQSLQTYVTQQLIRAAEIRASANLAATKMSECVLGQSKRVDFCGKGYHLMSFPQSA |
| | | PHGVVFLHVTYVPAQEKNFTTAPAICHDGKAHFPREGVFVSNGTHWFVTQRNFYEPQIITTDNTFVSGNCDVVI |
| | | GIVNNTVYDPLQPELDSFKEELDKYFKNHTSPDVDLGDISGINASVVNIQKEIDRLNEVAKNLNESLIDLQELGKY |
| | | EQYIKWPWYIWLGFIAGLIAIVMVTIMLCCMTSCCSCLKGCCSCGSCCKFDEDDSEPVLKGVKLHYT |
| 165 | RNA sequence | auguucguguuccuggugcugcugccucugguguccagccagugugugaaccugaucaccagaacacagUCAuacaccaacagc |
| | encoding a SARS- | uuuaccagaggcguguacuaccccgacaagguguucagauccagcgugcugcacucuacccaggaccuguuccugccuuucuuca |
| | COV-2 S protein | gcaacgugaccugguuccacgccauccacguguccggcaccaauggcaccaagagauucgacaaccccgcccugcccuucaacgac |
| | from an Omicron | gggguguacuuugccagcaccgagaaguccaacaucaucagaggcuggaucuucggcaccacacuggacagcaagacccagagcc |
| | XBB variant | ugcugaucgugaacaacgccaccaacguggucaucaaagugugcgaguuccaguucugcaacgaccccuuccuggacgucuacca |
| | | gaagaacaacaagagcuggauggaaagcgaguuccggguguacagcagcgccaacaacugcaccuucgaguacgugucccagccu |
| | | uuccugauggaccuggaaggcaaggagggcaacuucaagaaccugcgcgaguucguguuuaagaacaucgacggcuacuucaag |
| | | aucuacagcaagcacaccccuaucaaccucgagcgggaucugccucagggcuucucugcucuggaaccccugguggaucugccca |
| | | ucggcaucaacaucacccgguuucagacacugcuggcccugcacagaagcuaccugacaccuggcgauagcagcagcggauggac |
| | | agcuggugccgccgcuuacuaugugggcuaccugcagccuagaaccuuccugcugaaguacaacgagaacggcaccaucaccgac |
| | | gccguggauugugcucuggauccucugagcgagacaaagugcacccugaaguccuucaccguggaaaagggcaucuaccagacca |
| | | gcaacuuccgggugcagcccaccgaauccaucgugcgguuccccaauaucaccaaucugugccccuuccacgagguguucaaugcc |
| | | accaccuucgccucuguguacgccuggaaccggaagcggaucagcaauugcguggccgacuacuccgugaucuacaacuucgccc |
| | | ccuucuucgcauucaagugcuacggcguguccccuaccaagcugaacgaccugugcuucacaaacguguacgccgacagcuucgu |
| | | gauccggggaaacgaagugucacagauugccccuggacagacaggcaacaucgccgacuacaacuacaagcugcccgacgacuuc |
| | | accggcugugugauugccuggaacagcaacaagcuggacuccaaacccagcggcaacuacaauuaccuguaccggcuguuccgga |
| | | aguccaagcugaagcccuucgagcgggacaucuccaccgagaucuaucaggccggcaacaagccuuguaacggcguggcaggcag |
| | | caacugcuacagcccacugcaguccuacggcuuuaggcccacauacggcgugggccaccagcccuacagagugguggugcugagc |
| | | uucgaacugcugcaugccccugccacagugugcggcccuaagaaaagcaccaaucucgugaagaacaaaugcgugaacuucaacu |
| | | ucaacggccugaccggcaccggcgugcugacagagagcaacaagaaguuccugccauuccagcaguuuggccgggauaucgccga |
| | | uaccacagacgccguuagagauccccagacacuggaaauccuggacaucaccccuugcagcuucggcggagugucugugaucacc |
| | | ccuggcaccaacaccagcaaucagguggcagugcuguaccagggcgugaacuguaccgaagugcccguggccauucacgccgauc |
| | | agcugacaccuacauggcggguguacuccaccggcagcaauguguuucagaccagagccggcugucugaucggagccgaguacg |
| | | ugaacaauagcuacgagugcgacauccccaucggcgcuggaaucugcgccagcuaccagacacagacaaagagccaccggagagc |
| | | cagaagcguggccagccagagcaucauugccuacacaaugucucugggcgccgagaacagcguggccuacuccaacaacucuauc |
| | | gcuauccccaccaacuucaccaucagcgugaccacagagauccugccuguguccaugaccaagaccagcguggacugcaccaugua |
| | | caucugcggcgauuccaccgagugcuccaaccugcugcugcaguacggcagcuucugcacccagcugaaaagagcccugacaggg |
| | | aucgccguggaacaggacaagaacacccaagagguguucgcccaagugaagcagaucuacaagaccccuccuaucaaguacuucg |
| | | gcggcuucaauuucagccagauucugcccgauccuagcaagcccagcaagcggagcuucaucgaggaccugcuguucaacaaagu |
| | | gacacuggccgacgccggcuucaucaagcaguauggcgauugucugggcgacauugccgccagggaucugauuugcgcccagaa |
| | | guuuaacggacugacagugcugccuccucugcugaccgaugagaugaucgcccaguacacaucugcccugcuggccggcacaauc |
| | | acaagcggcuggacauuuggagcaggcgccgcucugcagauccccuuugcuaugcagauggccuaccgguucaacggcaucgga |
| | | gugacccagaaugugcuguacgagaaccagaagcugaucgccaaccaguucaacagcgccaucggcaagauccaggacagccuga |
| | | gcagcacagcaagcgcccugggaaagcugcaggacguggucaaccacaaugcccaggcacugaacacccuggucaagcagcugucc |
| | | uccaaguucggcgccaucagcucugugcugaacgauauccugagcagacuggacccuccugaggccgaggugcagaucgacagac |
| | | ugaucacaggcagacugcagagccuccagacauacgugacccagcagcugaucagagccgccgagauuagagccucugccaaucu |
| | | ggccgccaccaagaugucugagugugugcugggccagagcaagagaguggacuuuugcggcaagggcuaccaccugaugagcuu |
| | | cccucagucugccccucacggcgugguguuucugcacgugacauaugugcccgcucaagagaagaauuucaccaccgcuccagcc |
| | | aucugccacgacggcaaagcccacuuuccuagagaaggcguguucguguccaacggcacccauugguucgugacacagcggaacu |
| | | ucuacgagccccagaucaucaccaccgacaacaccuucgugucuggcaacugcgacgucgugaucggcauugugaacaauaccgu |
| | | guacgacccucugcagcccgagcuggacagcuucaaagaggaacuggacaaguacuuuaagaaccacacaagccccgacguggac |
| | | cugggcgauaucagcggaaucaaugccagcgucgugaacauccagaaagagaucgaccggcugaacgagguggccaagaaucug |
| | | aacgagagccugaucgaccugcaagaacuggggaaguacgagcaguacaucaaguggcccugguacaucuggcugggcuuuauc |
| | | gccggacugauugccaucgugauggucacaaucaugcuguguugcaugaccagcugcuguagcugccugaagggcuguuguagc |
| | | uguggcagcugcugcaaguucgacgaggacgauucugagcccgugcugaagggcgugaaacugcacuacacaugauga |
| 166 | DNA sequence | atgttcgtgttcctggtgctgctgcctctggtgtccagccagtgtgtgaacctgatcaccagaacacagTCAtacaccaacagctttaccagag |
| | encoding a SARS- | gcgtgtactaccccgacaaggtgttcagatccagcgtgctgcactctacccaggacctgttcctgcctttcttcagcaacgtgacctggttccac |
| | COV-2 S protein | gccatccacgtgtccggcaccaatggcaccaagagattcgacaaccccgccctgcccttcaacgacggggtgtactttgccagcaccgagaa |
| | from an Omicron | gtccaacatcatcagaggctggatcttcggcaccacactggacagcaagacccagagcctgctgatcgtgaacaacgccaccaacgtggtc |
| | XBB variant | atcaaagtgtgcgagttccagttctgcaacgaccccttcctggacgtctaccagaagaacaacaagagctggatggaaagcgagttccgggt |
| | | gtacagcagcgccaacaactgcaccttcgagtacgtgtcccagcctttcctgatggacctggaaggcaaggagggcaacttcaagaacctgc |
| | | gcgagttcgtgtttaagaacatcgacggctacttcaagatctacagcaagcacacccctatcaacctcgagcgggatctgcctcagggcttctc |
| | | tgctctggaacccctggtggatctgcccatcggcatcaacatcacccggtttcagacactgctggccctgcacagaagctacctgacacctggc |
| | | gatagcagcagcggatggacagctggtgccgccgcttactatgtgggctacctgcagcctagaaccttcctgctgaagtacaacgagaacgg |
| | | caccatcaccgacgccgtggattgtgctctggatcctctgagcgagacaaagtgcaccctgaagtccttcaccgtggaaaagggcatctacca |
| | | gaccagcaacttccgggtgcagcccaccgaatccatcgtgcggttccccaatatcaccaatctgtgccccttccacgaggtgttcaatgccacc |
| | | accttcgcctctgtgtacgcctggaaccggaagcggatcagcaattgcgtggccgactactccgtgatctacaacttcgcccccttcttcgcattc |
| | | aagtgctacggcgtgtcccctaccaagctgaacgacctgtgcttcacaaacgtgtacgccgacagcttcgtgatccggggaaacgaagtgtc |
| | | acagattgcccctggacagacaggcaacatcgccgactacaactacaagctgcccgacgacttcaccggctgtgtgattgcctggaacagca |
| | | acaagctggactccaaacccagcggcaactacaattacctgtaccggctgttccggaagtccaagctgaagcccttcgagcgggacatctcc |
| | | accgagatctatcaggccggcaacaagccttgtaacggcgtggcaggcagcaactgctacagcccactgcagtcctacggctttaggcccac |
| | | atacggcgtgggccaccagccctacagagtggtggtgctgagcttcgaactgctgcatgcccctgccacagtgtgcggccctaagaaaagca |
| | | ccaatctcgtgaagaacaaatgcgtgaacttcaacttcaacggcctgaccggcaccggcgtgctgacagagagcaacaagaagttcctgcc |
| | | attccagcagtttggccgggatatcgccgataccacagacgccgttagagatccccagacactggaaatcctggacatcaccccttgcagctt |
| | | cggcggagtgtctgtgatcacccctggcaccaacaccagcaatcaggtggcagtgctgtaccagggcgtgaactgtaccgaagtgcccgtg |
| | | gccattcacgccgatcagctgacacctacatggcgggtgtactccaccggcagcaatgtgtttcagaccagagccggctgtctgatcggagcc |
| | | gagtacgtgaacaatagctacgagtgcgacatccccatcggcgctggaatctgcgccagctaccagacacagacaaagagccaccggaga |
| | | gccagaagcgtggccagccagagcatcattgcctacacaatgtctctgggcgccgagaacagcgtggcctactccaacaactctatcgctatc |
| | | cccaccaacttcaccatcagcgtgaccacagagatcctgcctgtgtccatgaccaagaccagcgtggactgcaccatgtacatctgcggcgat |
| | | tccaccgagtgctccaacctgctgctgcagtacggcagcttctgcacccagctgaaaagagccctgacagggatcgccgtggaacaggaca |
| | | agaacacccaagaggtgttcgcccaagtgaagcagattacaagacccctcctatcaagtacttcggcggcttcaatttcagccagattctgcc |
| | | cgatcctagcaagcccagcaagcggagcttcatcgaggacctgctgttcaacaaagtgacactggccgacgccggcttcatcaagcagtatg |
| | | gcgattgtctgggcgacattgccgccagggatctgatttgcgcccagaagtttaacggactgacagtgctgcctcctctgctgaccgatgagat |
| | | gatcgcccagtacacatctgccctgctggccggcacaatcacaagcggctggacatttggagcaggcgccgctctgcagatcccctttgctat |
| | | gcagatggcctaccggttcaacggcatcggagtgacccagaatgtgctgtacgagaaccagaagctgatcgccaaccagttcaacagcgcc |
| | | atcggcaagatccaggacagcctgagcagcacagcaagcgccctgggaaagctgcaggacgtggtcaaccacaatgcccaggcactgaa |
| | | caccctggtcaagcagctgtcctccaagttcggcgccatcagctctgtgctgaacgatatcctgagcagactggaccctcctgaggccgaggt |
| | | gcagatcgacagactgatcacaggcagactgcagagcctccagacatacgtgacccagcagctgatcagagccgccgagattagagcctct |
| | | gccaatctggccgccaccaagatgtctgagtgtgtgctgggccagagcaagagagtggacttttgcggcaagggctaccacctgatgagctt |
| | | ccctcagtctgcccctcacggcgtggtgtttctgcacgtgacatatgtgcccgctcaagagaagaatttcaccaccgctccagccatctgccacg |
| | | acggcaaagcccactttcctagagaaggcgtgttcgtgtccaacggcacccattggttcgtgacacagcggaacttctacgagccccagatca |
| | | tcaccaccgacaacaccttcgtgtctggcaactgcgacgtcgtgatcggcattgtgaacaataccgtgtacgaccctctgcagcccgagctgg |
| | | acagcttcaaagaggaactggacaagtactttaagaaccacacaagccccgacgtggacctgggcgatatcagcggaatcaatgccagcgt |
| | | cgtgaacatccagaaagagatcgaccggctgaacgaggtggccaagaatctgaacgagagcctgatcgacctgcaagaactggggaagt |
| | | acgagcagtacatcaagtggccctggtacatctggctgggctttatcgccggactgattgccatcgtgatggtcacaatcatgctgtgttgcatg |
| | | accagctgctgtagctgcctgaagggctgttgtagctgtggcagctgctgcaagttcgacgaggacgattctgagcccgtgctgaagggcgt |
| | | gaaactgcactacacatgatga |
| 167 | Full length RNA | agaauaaacuaguauucuucugguccccacagacucagagagaacccgccaccauguucguguuccuggugcugcugccucugg |
| | construct encoding | uguccagccagugugugaaccugaucaccagaacacagUCAuacaccaacagcuuuaccagaggcguguacuaccccgacaagg |
| | a SARS-COV-2 S | uguucagauccagcgugcugcacucuacccaggaccuguuccugccuuucuucagcaacgugaccugguuccacgccauccacgu |
| | protein from an | guccggcaccaauggcaccaagagauucgacaaccccgcccugcccuucaacgacgggguguacuuugccagcaccgagaagucca |
| | Omicron XBB variant | acaucaucagaggcuggaucuucggcaccacacuggacagcaagacccagagccugcugaucgugaacaacgccaccaacguggu |
| | | caucaaagugugcgaguuccaguucugcaacgaccccuuccuggacgucuaccagaagaacaacaagagcuggauggaaagcga |
| | | guuccggguguacagcagcgccaacaacugcaccuucgaguacgugucccagccuuuccugauggaccuggaaggcaaggagggc |
| | | aacuucaagaaccugcgcgaguucguguuuaagaacaucgacggcuacuucaagaucuacagcaagcacaccccuaucaaccucg |
| | | agcgggaucugccucagggcuucucugcucuggaaccccugguggaucugcccaucggcaucaacaucacccgguuucagacacu |
| | | gcuggcccugcacagaagcuaccugacaccuggcgauagcagcagcggauggacagcuggugccgccgcuuacuaugugggcuac |
| | | cugcagccuagaaccuuccugcugaaguacaacgagaacggcaccaucaccgacgccguggauugugcucuggauccucugagcg |
| | | agacaaagugcacccugaaguccuucaccguggaaaagggcaucuaccagaccagcaacuuccgggugcagcccaccgaauccauc |
| | | gugcgguuccccaauaucaccaaucugugccccuuccacgagguguucaaugccaccaccuucgccucuguguacgccuggaaccg |
| | | gaagcggaucagcaauugcguggccgacuacuccgugaucuacaacuucgcccccuucuucgcauucaagugcuacggcgugucc |
| | | ccuaccaagcugaacgaccugugcuucacaaacguguacgccgacagcuucgugauccggggaaacgaagugucacagauugccc |
| | | cuggacagacaggcaacaucgccgacuacaacuacaagcugcccgacgacuucaccggcugugugauugccuggaacagcaacaa |
| | | gcuggacuccaaacccagcggcaacuacaauuaccuguaccggcuguuccggaaguccaagcugaagcccuucgagcgggacauc |
| | | uccaccgagaucuaucaggccggcaacaagccuuguaacggcguggcaggcagcaacugcuacagcccacugcaguccuacggcu |
| | | uuaggcccacauacggcgugggccaccagcccuacagagugguggugcugagcuucgaacugcugcaugccccugccacagugug |
| | | cggcccuaagaaaagcaccaaucucgugaagaacaaaugcgugaacuucaacuucaacggccugaccggcaccggcgugcugaca |
| | | gagagcaacaagaaguuccugccauuccagcaguuuggccgggauaucgccgauaccacagacgccguuagagauccccagacac |
| | | uggaaauccuggacaucaccccuugcagcuucggcggagugucugugaucaccccuggcaccaacaccagcaaucagguggcagu |
| | | gcuguaccagggcgugaacuguaccgaagugcccguggccauucacgccgaucagcugacaccuacauggcggguguacuccacc |
| | | ggcagcaauguguuucagaccagagccggcugucugaucggagccgaguacgugaacaauagcuacgagugcgacauccccauc |
| | | ggcgcuggaaucugcgccagcuaccagacacagacaaagagccaccggagagccagaagcguggccagccagagcaucauugccu |
| | | acacaaugucucugggcgccgagaacagcguggccuacuccaacaacucuaucgcuauccccaccaacuucaccaucagcgugacc |
| | | acagagauccugccuguguccaugaccaagaccagcguggacugcaccauguacaucugcggcgauuccaccgagugcuccaacc |
| | | ugcugcugcaguacggcagcuucugcacccagcugaaaagagcccugacagggaucgccguggaacaggacaagaacacccaaga |
| | | gguguucgcccaagugaagcagaucuacaagaccccuccuaucaaguacuucggcggcuucaauuucagccagauucugcccgau |
| | | ccuagcaagcccagcaagcggagcuucaucgaggaccugcuguucaacaaagugacacuggccgacgccggcuucaucaagcagu |
| | | auggcgauugucugggcgacauugccgccagggaucugauuugcgcccagaaguuuaacggacugacagugcugccuccucugc |
| | | ugaccgaugagaugaucgcccaguacacaucugcccugcuggccggcacaaucacaagcggcuggacauuuggagcaggcgccgc |
| | | ucugcagauccccuuugcuaugcagauggccuaccgguucaacggcaucggagugacccagaaugugcuguacgagaaccagaa |
| | | gcugaucgccaaccaguucaacagcgccaucggcaagauccaggacagccugagcagcacagcaagcgcccugggaaagcugcag |
| | | gacguggucaaccacaaugcccaggcacugaacacccuggucaagcagcuguccuccaaguucggcgccaucagcucugugcuga |
| | | acgauauccugagcagacuggacccuccugaggccgaggugcagaucgacagacugaucacaggcagacugcagagccuccagac |
| | | auacgugacccagcagcugaucagagccgccgagauuagagccucugccaaucuggccgccaccaagaugucugagugugugcug |
| | | ggccagagcaagagaguggacuuuugcggcaagggcuaccaccugaugagcuucccucagucugccccucacggcgugguguuu |
| | | cugcacgugacauaugugcccgcucaagagaagaauuucaccaccgcuccagccaucugccacgacggcaaagcccacuuuccuag |
| | | agaaggcguguucguguccaacggcacccauugguucgugacacagcggaacuucuacgagccccagaucaucaccaccgacaac |
| | | accuucgugucuggcaacugcgacgucgugaucggcauugugaacaauaccguguacgacccucugcagcccgagcuggacagcu |
| | | ucaaagaggaacuggacaaguacuuuaagaaccacacaagccccgacguggaccugggcgauaucagcggaaucaaugccagcgu |
| | | cgugaacauccagaaagagaucgaccggcugaacgagguggccaagaaucugaacgagagccugaucgaccugcaagaacuggg |
| | | gaaguacgagcaguacaucaaguggcccugguacaucuggcugggcuuuaucgccggacugauugccaucgugauggucacaau |
| | | caugcuguguugcaugaccagcugcuguagcugccugaagggcuguuguagcuguggcagcugcugcaaguucgacgaggacg |
| | | auucugagcccgugcugaagggcgugaaacugcacuacacaugaugacucgagcugguacugcaugcacgcaaugcuagcugcc |
| | | ccuuucccguccuggguaccccgagucucccccgaccucgggucccagguaugcucccaccuccaccugccccacucaccaccucug |
| | | cuaguuccagacaccucccaagcacgcagcaaugcagcucaaaacgcuuagccuagccacacccccacgggaaacagcagugauua |
| | | accuuuagcaauaaacgaaaguuuaacuaagcuauacuaaccccaggguuggucaauuucgugccagccacacccuggagcuagc |
| | | aaaaaaaaaaaaaaaaaaaaaaaaaaaaaagcauaugacuaaaaaaaaaaaaaaaaaaaaaaaaaaaaaaaaaaaaaaaaaaaa |
| | | aaaaaaaaaaaaaaaaaaaaaaaaa |
| 168 | Full length DNA | agaataaactagtattcttctggtccccacagactcagagagaacccgccaccatgttcgtgttcctggtgctgctgcctctggtgtccagccag |
| | construct encoding | tgtgtgaacctgatcaccagaacacagTCAtacaccaacagctttaccagaggcgtgtactaccccgacaaggtgttcagatccagcgtgct |
| | a SARS-COV-2 S | gcactctacccaggacctgttcctgcctttcttcagcaacgtgacctggttccacgccatccacgtgtccggcaccaatggcaccaagagattcg |
| | protein from an | acaaccccgccctgcccttcaacgacggggtgtactttgccagcaccgagaagtccaacatcatcagaggctggatcttcggcaccacactgg |
| | Omicron XBB variant | acagcaagacccagagcctgctgatcgtgaacaacgccaccaacgtggtcatcaaagtgtgcgagttccagttctgcaacgaccccttcctgg |
| | | acgtctaccagaagaacaacaagagctggatggaaagcgagttccgggtgtacagcagcgccaacaactgcaccttcgagtacgtgtccca |
| | | gcctttcctgatggacctggaaggcaaggagggcaacttcaagaacctgcgcgagttcgtgtttaagaacatcgacggctacttcaagatcta |
| | | cagcaagcacacccctatcaacctcgagcgggatctgcctcagggcttctctgctctggaacccctggtggatctgcccatcggcatcaacatc |
| | | acccggtttcagacactgctggccctgcacagaagctacctgacacctggcgatagcagcagcggatggacagctggtgccgccgcttacta |
| | | tgtgggctacctgcagcctagaaccttcctgctgaagtacaacgagaacggcaccatcaccgacgccgtggattgtgctctggatcctctgag |
| | | cgagacaaagtgcaccctgaagtccttcaccgtggaaaagggcatctaccagaccagcaacttccgggtgcagcccaccgaatccatcgtgc |
| | | ggttccccaatatcaccaatctgtgccccttccacgaggtgttcaatgccaccaccttcgcctctgtgtacgcctggaaccggaagcggatcagc |
| | | aattgcgtggccgactactccgtgatctacaacttcgcccccttcttcgcattcaagtgctacggcgtgtcccctaccaagctgaacgacctgtgc |
| | | ttcacaaacgtgtacgccgacagcttcgtgatccggggaaacgaagtgtcacagattgcccctggacagacaggcaacatcgccgactaca |
| | | actacaagctgcccgacgacttcaccggctgtgtgattgcctggaacagcaacaagctggactccaaacccagcggcaactacaattacctgt |
| | | accggctgttccggaagtccaagctgaagcccttcgagcgggacatctccaccgagatctatcaggccggcaacaagccttgtaacggcgtg |
| | | gcaggcagcaactgctacagcccactgcagtcctacggctttaggcccacatacggcgtgggccaccagccctacagagtggtggtgctga |
| | | gcttcgaactgctgcatgcccctgccacagtgtgcggccctaagaaaagcaccaatctcgtgaagaacaaatgcgtgaacttcaacttcaacg |
| | | gcctgaccggcaccggcgtgctgacagagagcaacaagaagttcctgccattccagcagtttggccgggatatcgccgataccacagacgc |
| | | cgttagagatccccagacactggaaatcctggacatcaccccttgcagcttcggcggagtgtctgtgatcacccctggcaccaacaccagcaa |
| | | tcaggtggcagtgctgtaccagggcgtgaactgtaccgaagtgcccgtggccattcacgccgatcagctgacacctacatggcgggtgtactc |
| | | caccggcagcaatgtgtttcagaccagagccggctgtctgatcggagccgagtacgtgaacaatagctacgagtgcgacatccccatcggcg |
| | | ctggaatctgcgccagctaccagacacagacaaagagccaccggagagccagaagcgtggccagccagagcatcattgcctacacaatgt |
| | | ctctgggcgccgagaacagcgtggcctactccaacaactctatcgctatccccaccaacttcaccatcagcgtgaccacagagatcctgcctgt |
| | | gtccatgaccaagaccagcgtggactgcaccatgtacatctgcggcgattccaccgagtgctccaacctgctgctgcagtacggcagcttctg |
| | | cacccagctgaaaagagccctgacagggatcgccgtggaacaggacaagaacacccaagaggtgttcgcccaagtgaagcagatctaca |
| | | agacccctcctatcaagtacttcggcggcttcaatttcagccagattctgcccgatcctagcaagcccagcaagcggagcttcatcgaggacct |
| | | gctgttcaacaaagtgacactggccgacgccggcttcatcaagcagtatggcgattgtctgggcgacattgccgccagggatctgatttgcgc |
| | | ccagaagtttaacggactgacagtgctgcctcctctgctgaccgatgagatgatcgcccagtacacatctgccctgctggccggcacaatcac |
| | | aagcggctggacatttggagcaggcgccgctctgcagatcccctttgctatgcagatggcctaccggttcaacggcatcggagtgacccaga |
| | | atgtgctgtacgagaaccagaagctgatcgccaaccagttcaacagcgccatcggcaagatccaggacagcctgagcagcacagcaagcg |
| | | ccctgggaaagctgcaggacgtggtcaaccacaatgcccaggcactgaacaccctggtcaagcagctgtcctccaagttcggcgccatcagc |
| | | tctgtgctgaacgatatcctgagcagactggaccctcctgaggccgaggtgcagatcgacagactgatcacaggcagactgcagagcctcca |
| | | gacatacgtgacccagcagctgatcagagccgccgagattagagcctctgccaatctggccgccaccaagatgtctgagtgtgtgctgggcc |
| | | agagcaagagagtggacttttgcggcaagggctaccacctgatgagcttccctcagtctgcccctcacggcgtggtgtttctgcacgtgacata |
| | | tgtgcccgctcaagagaagaatttcaccaccgctccagccatctgccacgacggcaaagcccactttcctagagaaggcgtgttcgtgtccaa |
| | | cggcacccattggttcgtgacacagcggaacttctacgagccccagatcatcaccaccgacaacaccttcgtgtctggcaactgcgacgtcgt |
| | | gatcggcattgtgaacaataccgtgtacgaccctctgcagcccgagctggacagcttcaaagaggaactggacaagtactttaagaaccaca |
| | | caagccccgacgtggacctgggcgatatcagcggaatcaatgccagcgtcgtgaacatccagaaagagatcgaccggctgaacgaggtgg |
| | | ccaagaatctgaacgagagcctgatcgacctgcaagaactggggaagtacgagcagtacatcaagtggccctggtacatctggctgggcttt |
| | | atcgccggactgattgccatcgtgatggtcacaatcatgctgtgttgcatgaccagctgctgtagctgcctgaagggctgttgtagctgtggca |
| | | gctgctgcaagttcgacgaggacgattctgagcccgtgctgaagggcgtgaaactgcactacacatgatgactcgagctggtactgcatgca |
| | | cgcaatgctagctgcccctttcccgtcctgggtaccccgagtctcccccgacctcgggtcccaggtatgctcccacctccacctgccccactcacc |
| | | acctctgctagttccagacacctcccaagcacgcagcaatgcagctcaaaacgcttagcctagccacacccccacgggaaacagcagtgatt |
| | | aacctttagcaataaacgaaagtttaactaagctatactaaccccagggttggtcaatttcgtgccagccacaccctggagctagcaaaaaaa |
| | | aaaaaaaaaaaaaaaaaaaaaaagcatatgactaaaaaaaaaaaaaaaaaaaaaaaaaaaaaaaaaaaaaaaaaaaaaaaaaaaa |
| | | aaaaaaaaaaaaaaaaa |

TABLE 16
Sequence of one embodiment of an exemplary Omicron
BQ.1.1-specific RNA vaccine

| SEQ | | |
|---|---|---|
| ID | | |
| NO. | Brief Description | Sequence |
| 169 | Amino acid sequence of RNA-encoded | MFVFLVLLPLVSSQCVNLITRTQSYTNSFTRGVYYPDKVF |
| | SARS-CoV-2 S protein from an | RSSVLHSTQDLFLPFFSNVTWFHAISGTNGTKRFDNPVLP |
| | Omicron BQ.1.1 variant (with PRO | FNDGVYFASTEKSNIIRGWIFGTTLDSKTQSLLIVNNATN |
| | mutations at positions corresponding | VVIKVCEFQFCNDPFLDVYYHKNNKSWMESEFRVYSSANN |
| | to K986P and V987P of SEQ ID NO: 1; | CTFEYVSQPFLMDLEGKQGNFKNLREFVFKNIDGYFKIYS |
| | i.e., PRO mutations at positions 981 | KHTPINLGRDLPQGFSALEPLVDLPIGINITRFQTLLALH |
| | and 982 of SEQ ID NO: 100) | RSYLTPGDSSSGWTAGAAAYYVGYLQPRTFLLKYNENGTI |
| | | TDAVDCALDPLSETKCTLKSFTVEKGIYQTSNFRVQPTES |
| | | IVRFPNITNLCPFDEVFNATTFASVYAWNRKRISNCVADY |
| | | SVLYNFAPFFAFKCYGVSPTKLNDLCFTNVYADSFVIRGN |
| | | EVSQIAPGQTGNIADYNYKLPDDFTGCVIAWNSNKLDSTV |
| | | GGNYNYRYRLFRKSKLKPFERDISTEIYQAGNKPCNGVAG |
| | | VNCYFPLQSYGFRPTYGVGHQPYRVVVLSFELLHAPATVC |
| | | GPKKSTNLVKNKCVNFNFNGLTGTGVLTESNKKFLPFQQF |
| | | GRDIADTTDAVRDPQTLEILDITPCSFGGVSVITPGTNTS |
| | | NQVAVLYQGVNCTEVPVAIHADQLTPTWRVYSTGSNVFQT |
| | | RAGCLIGAEYVNNSYECDIPIGAGICASYQTQTKSHRRAR |
| | | SVASQSIIAYTMSLGAENSVAYSNNSIAIPTNFTISVTTE |
| | | ILPVSMTKTSVDCTMYICGDSTECSNLLLQYGSFCTQLKR |
| | | ALTGIAVEQDKNTQEVFAQVKQIYKTPPIKYFGGFNFSQI |
| | | LPDPSKPSKRSFIEDLLFNKVTLADAGFIKQYGDCLGDIA |
| | | ARDLICAQKFNGLTVLPPLLTDEMIAQYTSALLAGTITSG |
| | | WTFGAGAALQIPFAMQMAYRFNGIGVTQNVLYENQKLIAN |
| | | QFNSAIGKIQDSLSSTASALGKLQDVVNHNAQALNTLVKQ |
| | | LSSKFGAISSVLNDILSRLDPPEAEVQIDRLITGRLQSLQ |
| | | TYVTQQLIRAAEIRASANLAATKMSECVLGQSKRVDFCGK |
| | | GYHLMSFPQSAPHGVVFLHVTYVPAQEKNFTTAPAICHDG |
| | | KAHFPREGVFVSNGTHWFVTQRNFYEPQIITTDNTFVSGN |
| | | CDVVIGIVNNTVYDPLQPELDSFKEELDKYFKNHTSPDVD |
| | | LGDISGINASVVNIQKEIDRLNEVAKNLNESLIDLQELGK |
| | | YEQYIKWPWYIWLGFIAGLIAIVMVTIMLCCMTSCCSCLK |
| | | GCCSCGSCCKFDEDDSEPVLKGVKLHYT |
| 170 | RNA sequence encoding a SARS-CoV-2 | AUGUUCGUGUUCCUGGUGCUGCUGCCUCUGGUGUCCAGCC |
| | S protein from an Omicron BQ.1.1 | AGUGUGUGAACCUGAUCACCAGAACACAGUCAUACACCAA |
| | variant | CAGCUUUACCAGAGGCGUGUACUACCCCGACAAGGUGUUC |
| | | AGAUCCAGCGUGCUGCACUCUACCCAGGACCUGUUCCUGC |
| | | CUUUCUUCAGCAACGUGACCUGGUUCCACGCCAUCUCCGG |
| | | CACCAAUGGCACCAAGAGAUUCGACAACCCCGUGCUGCCC |
| | | UUCAACGACGGGGUGUACUUUGCCAGCACCGAGAAGUCCA |
| | | ACAUCAUCAGAGGCUGGAUCUUCGGCACCACACUGGACAG |
| | | CAAGACCCAGAGCCUGCUGAUCGUGAACAACGCCACCAAC |
| | | GUGGUCAUCAAAGUGUGCGAGUUCCAGUUCUGCAACGACC |
| | | CCUUCCUGGACGUCUACUACCACAAGAACAACAAGAGCUG |
| | | GAUGGAAAGCGAGUUCCGGGUGUACAGCAGCGCCAACAAC |
| | | UGCACCUUCGAGUACGUGUCCCAGCCUUUCCUGAUGGACC |
| | | UGGAAGGCAAGCAGGGCAACUUCAAGAACCUGCGCGAGUU |
| | | CGUGUUUAAGAACAUCGACGGCUACUUCAAGAUCUACAGC |
| | | AAGCACACCCCUAUCAACCUCGGCCGGGAUCUGCCUCAGG |
| | | GCUUCUCUGCUCUGGAACCCCUGGUGGAUCUGCCCAUCGG |
| | | CAUCAACAUCACCCGGUUUCAGACACUGCUGGCCCUGCAC |
| | | AGAAGCUACCUGACACCUGGCGAUAGCAGCAGCGGAUGGA |
| | | CAGCUGGUGCCGCCGCUUACUAUGUGGGCUACCUGCAGCC |
| | | UAGAACCUUCCUGCUGAAGUACAACGAGAACGGCACCAUC |
| | | ACCGACGCCGUGGAUUGUGCUCUGGAUCCUCUGAGCGAGA |
| | | CAAAGUGCACCCUGAAGUCCUUCACCGUGGAAAAGGGCAU |
| | | CUACCAGACCAGCAACUUCCGGGUGCAGCCCACCGAAUCC |
| | | AUCGUGCGGUUCCCCAAUAUCACCAAUCUGUGCCCCUUCG |
| | | ACGAGGUGUUCAAUGCCACCACCUUCGCCUCUGUGUACGC |
| | | CUGGAACCGGAAGCGGAUCAGCAAUUGCGUGGCCGACUAC |
| | | UCCGUGCUGUACAACUUCGCCCCCUUCUUCGCAUUCAAGU |
| | | GCUACGGCGUGUCCCCUACCAAGCUGAACGACCUGUGCUU |
| | | CACAAACGUGUACGCCGACAGCUUCGUGAUCCGGGGAAAC |
| | | GAAGUGUCACAGAUUGCCCCUGGACAGACAGGCAACAUCG |
| | | CCGACUACAACUACAAGCUGCCCGACGACUUCACCGGCUG |
| | | UGUGAUUGCCUGGAACAGCAACAAGCUGGACUCCACCGUC |
| | | GGCGGCAACUACAAUUACAGGUACCGGCUGUUCCGGAAGU |
| | | CCAAGCUGAAGCCCUUCGAGCGGGACAUCUCCACCGAGAU |
| | | CUAUCAGGCCGGCAACAAGCCUUGUAACGGCGUGGCAGGC |
| | | GUGAACUGCUACUUCCCACUGCAGUCCUACGGCUUUAGGC |
| | | CCACAUACGGCGUGGGCCACCAGCCCUACAGAGUGGUGGU |
| | | GCUGAGCUUCGAACUGCUGCAUGCCCCUGCCACAGUGUGC |
| | | GGCCCUAAGAAAAGCACCAAUCUCGUGAAGAACAAAUGCG |
| | | UGAACUUCAACUUCAACGGCCUGACCGGCACCGGCGUGCU |
| | | GACAGAGAGCAACAAGAAGUUCCUGCCAUUCCAGCAGUUU |
| | | GGCCGGGAUAUCGCCGAUACCACAGACGCCGUUAGAGAUC |
| | | CCCAGACACUGGAAAUCCUGGACAUCACCCCUUGCAGCUU |
| | | CGGCGGAGUGUCUGUGAUCACCCCUGGCACCAACACCAGC |
| | | AAUCAGGUGGCAGUGCUGUACCAGGGCGUGAACUGUACCG |
| | | AAGUGCCCGUGGCCAUUCACGCCGAUCAGCUGACACCUAC |
| | | AUGGCGGGUGUACUCCACCGGCAGCAAUGUGUUUCAGACC |
| | | AGAGCCGGCUGUCUGAUCGGAGCCGAGUACGUGAACAAUA |
| | | GCUACGAGUGCGACAUCCCCAUCGGCGCUGGAAUCUGCGC |
| | | CAGCUACCAGACACAGACAAAGAGCCACCGGAGAGCCAGA |
| | | AGCGUGGCCAGCCAGAGCAUCAUUGCCUACACAAUGUCUC |
| | | UGGGCGCCGAGAACAGCGUGGCCUACUCCAACAACUCUAU |
| | | CGCUAUCCCCACCAACUUCACCAUCAGCGUGACCACAGAG |
| | | AUCCUGCCUGUGUCCAUGACCAAGACCAGCGUGGACUGCA |
| | | CCAUGUACAUCUGCGGCGAUUCCACCGAGUGCUCCAACCU |
| | | GCUGCUGCAGUACGGCAGCUUCUGCACCCAGCUGAAAAGA |
| | | GCCCUGACAGGGAUCGCCGUGGAACAGGACAAGAACACCC |
| | | AAGAGGUGUUCGCCCAAGUGAAGCAGAUCUACAAGACCCC |
| | | UCCUAUCAAGUACUUCGGCGGCUUCAAUUUCAGCCAGAUU |
| | | CUGCCCGAUCCUAGCAAGCCCAGCAAGCGGAGCUUCAUCG |
| | | AGGACCUGCUGUUCAACAAAGUGACACUGGCCGACGCCGG |
| | | CUUCAUCAAGCAGUAUGGCGAUUGUCUGGGCGACAUUGCC |
| | | GCCAGGGAUCUGAUUUGCGCCCAGAAGUUUAACGGACUGA |
| | | CAGUGCUGCCUCCUCUGCUGACCGAUGAGAUGAUCGCCCA |
| | | GUACACAUCUGCCCUGCUGGCCGGCACAAUCACAAGCGGC |
| | | UGGACAUUUGGAGCAGGCGCCGCUCUGCAGAUCCCCUUUG |
| | | CUAUGCAGAUGGCCUACCGGUUCAACGGCAUCGGAGUGAC |
| | | CCAGAAUGUGCUGUACGAGAACCAGAAGCUGAUCGCCAAC |
| | | CAGUUCAACAGCGCCAUCGGCAAGAUCCAGGACAGCCUGA |
| | | GCAGCACAGCAAGCGCCCUGGGAAAGCUGCAGGACGUGGU |
| | | CAACCACAAUGCCCAGGCACUGAACACCCUGGUCAAGCAG |
| | | CUGUCCUCCAAGUUCGGCGCCAUCAGCUCUGUGCUGAACG |
| | | AUAUCCUGAGCAGACUGGACCCUCCUGAGGCCGAGGUGCA |
| | | GAUCGACAGACUGAUCACAGGCAGACUGCAGAGCCUCCAG |
| | | ACAUACGUGACCCAGCAGCUGAUCAGAGCCGCCGAGAUUA |
| | | GAGCCUCUGCCAAUCUGGCCGCCACCAAGAUGUCUGAGUG |
| | | UGUGCUGGGCCAGAGCAAGAGAGUGGACUUUUGCGGCAAG |
| | | GGCUACCACCUGAUGAGCUUCCCUCAGUCUGCCCCUCACG |
| | | GCGUGGUGUUUCUGCACGUGACAUAUGUGCCCGCUCAAGA |
| | | GAAGAAUUUCACCACCGCUCCAGCCAUCUGCCACGACGGC |
| | | AAAGCCCACUUUCCUAGAGAAGGCGUGUUCGUGUCCAACG |
| | | GCACCCAUUGGUUCGUGACACAGCGGAACUUCUACGAGCC |
| | | CCAGAUCAUCACCACCGACAACACCUUCGUGUCUGGCAAC |
| | | UGCGACGUCGUGAUCGGCAUUGUGAACAAUACCGUGUACG |
| | | ACCCUCUGCAGCCCGAGCUGGACAGCUUCAAAGAGGAACU |
| | | GGACAAGUACUUUAAGAACCACACAAGCCCCGACGUGGAC |
| | | CUGGGCGAUAUCAGCGGAAUCAAUGCCAGCGUCGUGAACA |
| | | UCCAGAAAGAGAUCGACCGGCUGAACGAGGUGGCCAAGAA |
| | | UCUGAACGAGAGCCUGAUCGACCUGCAAGAACUGGGGAAG |
| | | UACGAGCAGUACAUCAAGUGGCCCUGGUACAUCUGGCUGG |
| | | GCUUUAUCGCCGGACUGAUUGCCAUCGUGAUGGUCACAAU |
| | | CAUGCUGUGUUGCAUGACCAGCUGCUGUAGCUGCCUGAAG |
| | | GGCUGUUGUAGCUGUGGCAGCUGCUGCAAGUUCGACGAGG |
| | | ACGAUUCUGAGCCCGUGCUGAAGGGCGUGAAACUGCACUA |
| | | CACAUGAUGA |
| 171 | DNA sequence encoding a SARS-CoV-2 | ATGTTCGTGTTCCTGGTGCTGCTGCCTCTGGTGTCCAGCC |
| | S protein from an Omicron BQ.1.1 | AGTGTGTGAACCTGATCACCAGAACACAGTCATACACCAA |
| | variant | CAGCTTTACCAGAGGCGTGTACTACCCCGACAAGGTGTTC |
| | | AGATCCAGCGTGCTGCACTCTACCCAGGACCTGTTCCTGC |
| | | CTTTCTTCAGCAACGTGACCTGGTTCCACGCCATCTCCGG |
| | | CACCAATGGCACCAAGAGATTCGACAACCCCGTGCTGCCC |
| | | TTCAACGACGGGGTGTACTTTGCCAGCACCGAGAAGTCCA |
| | | ACATCATCAGAGGCTGGATCTTCGGCACCACACTGGACAG |
| | | CAAGACCCAGAGCCTGCTGATCGTGAACAACGCCACCAAC |
| | | GTGGTCATCAAAGTGTGCGAGTTCCAGTTCTGCAACGACC |
| | | CCTTCCTGGACGTCTACTACCACAAGAACAACAAGAGCTG |
| | | GATGGAAAGCGAGTTCCGGGTGTACAGCAGCGCCAACAAC |
| | | TGCACCTTCGAGTACGTGTCCCAGCCTTTCCTGATGGACC |
| | | TGGAAGGCAAGCAGGGCAACTTCAAGAACCTGCGCGAGTT |
| | | CGTGTTTAAGAACATCGACGGCTACTTCAAGATCTACAGC |
| | | AAGCACACCCCTATCAACCTCGGCCGGGATCTGCCTCAGG |
| | | GCTTCTCTGCTCTGGAACCCCTGGTGGATCTGCCCATCGG |
| | | CATCAACATCACCCGGTTTCAGACACTGCTGGCCCTGCAC |
| | | AGAAGCTACCTGACACCTGGCGATAGCAGCAGCGGATGGA |
| | | CAGCTGGTGCCGCCGCTTACTATGTGGGCTACCTGCAGCC |
| | | TAGAACCTTCCTGCTGAAGTACAACGAGAACGGCACCATC |
| | | ACCGACGCCGTGGATTGTGCTCTGGATCCTCTGAGCGAGA |
| | | CAAAGTGCACCCTGAAGTCCTTCACCGTGGAAAAGGGCAT |
| | | CTACCAGACCAGCAACTTCCGGGTGCAGCCCACCGAATCC |
| | | ATCGTGCGGTTCCCCAATATCACCAATCTGTGCCCCTTCG |
| | | ACGAGGTGTTCAATGCCACCACCTTCGCCTCTGTGTACGC |
| | | CTGGAACCGGAAGCGGATCAGCAATTGCGTGGCCGACTAC |
| | | TCCGTGCTGTACAACTTCGCCCCCTTCTTCGCATTCAAGT |
| | | GCTACGGCGTGTCCCCTACCAAGCTGAACGACCTGTGCTT |
| | | CACAAACGTGTACGCCGACAGCTTCGTGATCCGGGGAAAC |
| | | GAAGTGTCACAGATTGCCCCTGGACAGACAGGCAACATCG |
| | | CCGACTACAACTACAAGCTGCCCGACGACTTCACCGGCTG |
| | | TGTGATTGCCTGGAACAGCAACAAGCTGGACTCCACCGTC |
| | | GGCGGCAACTACAATTACAGGTACCGGCTGTTCCGGAAGT |
| | | CCAAGCTGAAGCCCTTCGAGCGGGACATCTCCACCGAGAT |
| | | CTATCAGGCCGGCAACAAGCCTTGTAACGGCGTGGCAGGC |
| | | GTGAACTGCTACTTCCCACTGCAGTCCTACGGCTTTAGGC |
| | | CCACATACGGCGTGGGCCACCAGCCCTACAGAGTGGTGGT |
| | | GCTGAGCTTCGAACTGCTGCATGCCCCTGCCACAGTGTGC |
| | | GGCCCTAAGAAAAGCACCAATCTCGTGAAGAACAAATGCG |
| | | TGAACTTCAACTTCAACGGCCTGACCGGCACCGGCGTGCT |
| | | GACAGAGAGCAACAAGAAGTTCCTGCCATTCCAGCAGTTT |
| | | GGCCGGGATATCGCCGATACCACAGACGCCGTTAGAGATC |
| | | CCCAGACACTGGAAATCCTGGACATCACCCCTTGCAGCTT |
| | | CGGCGGAGTGTCTGTGATCACCCCTGGCACCAACACCAGC |
| | | AATCAGGTGGCAGTGCTGTACCAGGGCGTGAACTGTACCG |
| | | AAGTGCCCGTGGCCATTCACGCCGATCAGCTGACACCTAC |
| | | ATGGCGGGTGTACTCCACCGGCAGCAATGTGTTTCAGACC |
| | | AGAGCCGGCTGTCTGATCGGAGCCGAGTACGTGAACAATA |
| | | GCTACGAGTGCGACATCCCCATCGGCGCTGGAATCTGCGC |
| | | CAGCTACCAGACACAGACAAAGAGCCACCGGAGAGCCAGA |
| | | AGCGTGGCCAGCCAGAGCATCATTGCCTACACAATGTCTC |
| | | TGGGCGCCGAGAACAGCGTGGCCTACTCCAACAACTCTAT |
| | | CGCTATCCCCACCAACTTCACCATCAGCGTGACCACAGAG |
| | | ATCCTGCCTGTGTCCATGACCAAGACCAGCGTGGACTGCA |
| | | CCATGTACATCTGCGGCGATTCCACCGAGTGCTCCAACCT |
| | | GCTGCTGCAGTACGGCAGCTTCTGCACCCAGCTGAAAAGA |
| | | GCCCTGACAGGGATCGCCGTGGAACAGGACAAGAACACCC |
| | | AAGAGGTGTTCGCCCAAGTGAAGCAGATCTACAAGACCCC |
| | | TCCTATCAAGTACTTCGGCGGCTTCAATTTCAGCCAGATT |
| | | CTGCCCGATCCTAGCAAGCCCAGCAAGCGGAGCTTCATCG |
| | | AGGACCTGCTGTTCAACAAAGTGACACTGGCCGACGCCGG |
| | | CTTCATCAAGCAGTATGGCGATTGTCTGGGCGACATTGCC |
| | | GCCAGGGATCTGATTTGCGCCCAGAAGTTTAACGGACTGA |
| | | CAGTGCTGCCTCCTCTGCTGACCGATGAGATGATCGCCCA |
| | | GTACACATCTGCCCTGCTGGCCGGCACAATCACAAGCGGC |
| | | TGGACATTTGGAGCAGGCGCCGCTCTGCAGATCCCCTTTG |
| | | CTATGCAGATGGCCTACCGGTTCAACGGCATCGGAGTGAC |
| | | CCAGAATGTGCTGTACGAGAACCAGAAGCTGATCGCCAAC |
| | | CAGTTCAACAGCGCCATCGGCAAGATCCAGGACAGCCTGA |
| | | GCAGCACAGCAAGCGCCCTGGGAAAGCTGCAGGACGTGGT |
| | | CAACCACAATGCCCAGGCACTGAACACCCTGGTCAAGCAG |
| | | CTGTCCTCCAAGTTCGGCGCCATCAGCTCTGTGCTGAACG |
| | | ATATCCTGAGCAGACTGGACCCTCCTGAGGCCGAGGTGCA |
| | | GATCGACAGACTGATCACAGGCAGACTGCAGAGCCTCCAG |
| | | ACATACGTGACCCAGCAGCTGATCAGAGCCGCCGAGATTA |
| | | GAGCCTCTGCCAATCTGGCCGCCACCAAGATGTCTGAGTG |
| | | TGTGCTGGGCCAGAGCAAGAGAGTGGACTTTTGCGGCAAG |
| | | GGCTACCACCTGATGAGCTTCCCTCAGTCTGCCCCTCACG |
| | | GCGTGGTGTTTCTGCACGTGACATATGTGCCCGCTCAAGA |
| | | GAAGAATTTCACCACCGCTCCAGCCATCTGCCACGACGGC |
| | | AAAGCCCACTTTCCTAGAGAAGGCGTGTTCGTGTCCAACG |
| | | GCACCCATTGGTTCGTGACACAGCGGAACTTCTACGAGCC |
| | | CCAGATCATCACCACCGACAACACCTTCGTGTCTGGCAAC |
| | | TGCGACGTCGTGATCGGCATTGTGAACAATACCGTGTACG |
| | | ACCCTCTGCAGCCCGAGCTGGACAGCTTCAAAGAGGAACT |
| | | GGACAAGTACTTTAAGAACCACACAAGCCCCGACGTGGAC |
| | | CTGGGCGATATCAGCGGAATCAATGCCAGCGTCGTGAACA |
| | | TCCAGAAAGAGATCGACCGGCTGAACGAGGTGGCCAAGAA |
| | | TCTGAACGAGAGCCTGATCGACCTGCAAGAACTGGGGAAG |
| | | TACGAGCAGTACATCAAGTGGCCCTGGTACATCTGGCTGG |
| | | GCTTTATCGCCGGACTGATTGCCATCGTGATGGTCACAAT |
| | | CATGCTGTGTTGCATGACCAGCTGCTGTAGCTGCCTGAAG |
| | | GGCTGTTGTAGCTGTGGCAGCTGCTGCAAGTTCGACGAGG |
| | | ACGATTCTGAGCCCGTGCTGAAGGGCGTGAAACTGCACTA |
| | | CACATGATGA |
| 172 | Full length RNA construct encoding a | Agaauaaacuaguauucuucugguccccacagacucagag |
| | SARS-CoV-2 S protein from an | agaacccgccaccAUGUUCGUGUUCCUGGUGCUGCUGCCU |
| | Omicron BQ.1.1 variant | CUGGUGUCCAGCCAGUGUGUGAACCUGAUCACCAGAACAC |
| | | AGUCAUACACCAACAGCUUUACCAGAGGCGUGUACUACCC |
| | | CGACAAGGUGUUCAGAUCCAGCGUGCUGCACUCUACCCAG |
| | | GACCUGUUCCUGCCUUUCUUCAGCAACGUGACCUGGUUCC |
| | | ACGCCAUCUCCGGCACCAAUGGCACCAAGAGAUUCGACAA |
| | | CCCCGUGCUGCCCUUCAACGACGGGGUGUACUUUGCCAGC |
| | | ACCGAGAAGUCCAACAUCAUCAGAGGCUGGAUCUUCGGCA |
| | | CCACACUGGACAGCAAGACCCAGAGCCUGCUGAUCGUGAA |
| | | CAACGCCACCAACGUGGUCAUCAAAGUGUGCGAGUUCCAG |
| | | UUCUGCAACGACCCCUUCCUGGACGUCUACUACCACAAGA |
| | | ACAACAAGAGCUGGAUGGAAAGCGAGUUCCGGGUGUACAG |
| | | CAGCGCCAACAACUGCACCUUCGAGUACGUGUCCCAGCCU |
| | | UUCCUGAUGGACCUGGAAGGCAAGCAGGGCAACUUCAAGA |
| | | ACCUGCGCGAGUUCGUGUUUAAGAACAUCGACGGCUACUU |
| | | CAAGAUCUACAGCAAGCACACCCCUAUCAACCUCGGCCGG |
| | | GAUCUGCCUCAGGGCUUCUCUGCUCUGGAACCCCUGGUGG |
| | | AUCUGCCCAUCGGCAUCAACAUCACCCGGUUUCAGACACU |
| | | GCUGGCCCUGCACAGAAGCUACCUGACACCUGGCGAUAGC |
| | | AGCAGCGGAUGGACAGCUGGUGCCGCCGCUUACUAUGUGG |
| | | GCUACCUGCAGCCUAGAACCUUCCUGCUGAAGUACAACGA |
| | | GAACGGCACCAUCACCGACGCCGUGGAUUGUGCUCUGGAU |
| | | CCUCUGAGCGAGACAAAGUGCACCCUGAAGUCCUUCACCG |
| | | UGGAAAAGGGCAUCUACCAGACCAGCAACUUCCGGGUGCA |
| | | GCCCACCGAAUCCAUCGUGCGGUUCCCCAAUAUCACCAAU |
| | | CUGUGCCCCUUCGACGAGGUGUUCAAUGCCACCACCUUCG |
| | | CCUCUGUGUACGCCUGGAACCGGAAGCGGAUCAGCAAUUG |
| | | CGUGGCCGACUACUCCGUGCUGUACAACUUCGCCCCCUUC |
| | | UUCGCAUUCAAGUGCUACGGCGUGUCCCCUACCAAGCUGA |
| | | ACGACCUGUGCUUCACAAACGUGUACGCCGACAGCUUCGU |
| | | GAUCCGGGGAAACGAAGUGUCACAGAUUGCCCCUGGACAG |
| | | ACAGGCAACAUCGCCGACUACAACUACAAGCUGCCCGACG |
| | | ACUUCACCGGCUGUGUGAUUGCCUGGAACAGCAACAAGCU |
| | | GGACUCCACCGUCGGCGGCAACUACAAUUACAGGUACCGG |
| | | CUGUUCCGGAAGUCCAAGCUGAAGCCCUUCGAGCGGGACA |
| | | UCUCCACCGAGAUCUAUCAGGCCGGCAACAAGCCUUGUAA |
| | | CGGCGUGGCAGGCGUGAACUGCUACUUCCCACUGCAGUCC |
| | | UACGGCUUUAGGCCCACAUACGGCGUGGGCCACCAGCCCU |
| | | ACAGAGUGGUGGUGCUGAGCUUCGAACUGCUGCAUGCCCC |
| | | UGCCACAGUGUGCGGCCCUAAGAAAAGCACCAAUCUCGUG |
| | | AAGAACAAAUGCGUGAACUUCAACUUCAACGGCCUGACCG |
| | | GCACCGGCGUGCUGACAGAGAGCAACAAGAAGUUCCUGCC |
| | | AUUCCAGCAGUUUGGCCGGGAUAUCGCCGAUACCACAGAC |
| | | GCCGUUAGAGAUCCCCAGACACUGGAAAUCCUGGACAUCA |
| | | CCCCUUGCAGCUUCGGCGGAGUGUCUGUGAUCACCCCUGG |
| | | CACCAACACCAGCAAUCAGGUGGCAGUGCUGUACCAGGGC |
| | | GUGAACUGUACCGAAGUGCCCGUGGCCAUUCACGCCGAUC |
| | | AGCUGACACCUACAUGGCGGGUGUACUCCACCGGCAGCAA |
| | | UGUGUUUCAGACCAGAGCCGGCUGUCUGAUCGGAGCCGAG |
| | | UACGUGAACAAUAGCUACGAGUGCGACAUCCCCAUCGGCG |
| | | CUGGAAUCUGCGCCAGCUACCAGACACAGACAAAGAGCCA |
| | | CCGGAGAGCCAGAAGCGUGGCCAGCCAGAGCAUCAUUGCC |
| | | UACACAAUGUCUCUGGGCGCCGAGAACAGCGUGGCCUACU |
| | | CCAACAACUCUAUCGCUAUCCCCACCAACUUCACCAUCAG |
| | | CGUGACCACAGAGAUCCUGCCUGUGUCCAUGACCAAGACC |
| | | AGCGUGGACUGCACCAUGUACAUCUGCGGCGAUUCCACCG |
| | | AGUGCUCCAACCUGCUGCUGCAGUACGGCAGCUUCUGCAC |
| | | CCAGCUGAAAAGAGCCCUGACAGGGAUCGCCGUGGAACAG |
| | | GACAAGAACACCCAAGAGGUGUUCGCCCAAGUGAAGCAGA |
| | | UCUACAAGACCCCUCCUAUCAAGUACUUCGGCGGCUUCAA |
| | | UUUCAGCCAGAUUCUGCCCGAUCCUAGCAAGCCCAGCAAG |
| | | CGGAGCUUCAUCGAGGACCUGCUGUUCAACAAAGUGACAC |
| | | UGGCCGACGCCGGCUUCAUCAAGCAGUAUGGCGAUUGUCU |
| | | GGGCGACAUUGCCGCCAGGGAUCUGAUUUGCGCCCAGAAG |
| | | UUUAACGGACUGACAGUGCUGCCUCCUCUGCUGACCGAUG |
| | | AGAUGAUCGCCCAGUACACAUCUGCCCUGCUGGCCGGCAC |
| | | AAUCACAAGCGGCUGGACAUUUGGAGCAGGCGCCGCUCUG |
| | | CAGAUCCCCUUUGCUAUGCAGAUGGCCUACCGGUUCAACG |
| | | GCAUCGGAGUGACCCAGAAUGUGCUGUACGAGAACCAGAA |
| | | GCUGAUCGCCAACCAGUUCAACAGCGCCAUCGGCAAGAUC |
| | | CAGGACAGCCUGAGCAGCACAGCAAGCGCCCUGGGAAAGC |
| | | UGCAGGACGUGGUCAACCACAAUGCCCAGGCACUGAACAC |
| | | CCUGGUCAAGCAGCUGUCCUCCAAGUUCGGCGCCAUCAGC |
| | | UCUGUGCUGAACGAUAUCCUGAGCAGACUGGACCCUCCUG |
| | | AGGCCGAGGUGCAGAUCGACAGACUGAUCACAGGCAGACU |
| | | GCAGAGCCUCCAGACAUACGUGACCCAGCAGCUGAUCAGA |
| | | GCCGCCGAGAUUAGAGCCUCUGCCAAUCUGGCCGCCACCA |
| | | AGAUGUCUGAGUGUGUGCUGGGCCAGAGCAAGAGAGUGGA |
| | | CUUUUGCGGCAAGGGCUACCACCUGAUGAGCUUCCCUCAG |
| | | UCUGCCCCUCACGGCGUGGUGUUUCUGCACGUGACAUAUG |
| | | UGCCCGCUCAAGAGAAGAAUUUCACCACCGCUCCAGCCAU |
| | | CUGCCACGACGGCAAAGCCCACUUUCCUAGAGAAGGCGUG |
| | | UUCGUGUCCAACGGCACCCAUUGGUUCGUGACACAGCGGA |
| | | ACUUCUACGAGCCCCAGAUCAUCACCACCGACAACACCUU |
| | | CGUGUCUGGCAACUGCGACGUCGUGAUCGGCAUUGUGAAC |
| | | AAUACCGUGUACGACCCUCUGCAGCCCGAGCUGGACAGCU |
| | | UCAAAGAGGAACUGGACAAGUACUUUAAGAACCACACAAG |
| | | CCCCGACGUGGACCUGGGCGAUAUCAGCGGAAUCAAUGCC |
| | | AGCGUCGUGAACAUCCAGAAAGAGAUCGACCGGCUGAACG |
| | | AGGUGGCCAAGAAUCUGAACGAGAGCCUGAUCGACCUGCA |
| | | AGAACUGGGGAAGUACGAGCAGUACAUCAAGUGGCCCUGG |
| | | UACAUCUGGCUGGGCUUUAUCGCCGGACUGAUUGCCAUCG |
| | | UGAUGGUCACAAUCAUGCUGUGUUGCAUGACCAGCUGCUG |
| | | UAGCUGCCUGAAGGGCUGUUGUAGCUGUGGCAGCUGCUGC |
| | | AAGUUCGACGAGGACGAUUCUGAGCCCGUGCUGAAGGGCG |
| | | UGAAACUGCACUACACAUGAUGAcucgagcugguacugca |
| | | ugcacgcaaugcuagcugccccuuucccguccuggguacc |
| | | ccgagucucccccgaccucgggucccagguaugcucccac |
| | | cuccaccugccccacucaccaccucugcuaguuccagaca |
| | | ccucccaagcacgcagcaaugcagcucaaaacgcuuagcc |
| | | uagccacacccccacgggaaacagcagugauuaaccuuua |
| | | gcaauaaacgaaaguuuaacuaagcuauacuaaccccagg |
| | | guuggucaauuucgugccagccacacccuggagcuagcaa |
| | | aaaaaaaaaaaaaaaaaaaaaaaaaaaagcauaugacuaa |
| | | aaaaaaaaaaaaaaaaaaaaaaaaaaaaaaaaaaaaaaaa |
| | | aaaaaaaaaaaaaaaaaaaaaaaaaaa |
| 173 | Full length DNA construct | Agaataaactagtattcttctggtccccacagactcagag |
| | encoding a SARS-CoV-2 S | agaacccgccaccATGTTCGTGTTCCTGGTGCTGCTGCCT |
| | protein from an Omicron | CTGGTGTCCAGCCAGTGTGTGAACCTGATCACCAGAACAC |
| | BQ.1.1 variant | AGTCATACACCAACAGCTTTACCAGAGGCGTGTACTACCC |
| | | CGACAAGGTGTTCAGATCCAGCGTGCTGCACTCTACCCAG |
| | | GACCTGTTCCTGCCTTTCTTCAGCAACGTGACCTGGTTCC |
| | | ACGCCATCTCCGGCACCAATGGCACCAAGAGATTCGACAA |
| | | CCCCGTGCTGCCCTTCAACGACGGGGTGTACTTTGCCAGC |
| | | ACCGAGAAGTCCAACATCATCAGAGGCTGGATCTTCGGCA |
| | | CCACACTGGACAGCAAGACCCAGAGCCTGCTGATCGTGAA |
| | | CAACGCCACCAACGTGGTCATCAAAGTGTGCGAGTTCCAG |
| | | TTCTGCAACGACCCCTTCCTGGACGTCTACTACCACAAGA |
| | | ACAACAAGAGCTGGATGGAAAGCGAGTTCCGGGTGTACAG |
| | | CAGCGCCAACAACTGCACCTTCGAGTACGTGTCCCAGCCT |
| | | TTCCTGATGGACCTGGAAGGCAAGCAGGGCAACTTCAAGA |
| | | ACCTGCGCGAGTTCGTGTTTAAGAACATCGACGGCTACTT |
| | | CAAGATCTACAGCAAGCACACCCCTATCAACCTCGGCCGG |
| | | GATCTGCCTCAGGGCTTCTCTGCTCTGGAACCCCTGGTGG |
| | | ATCTGCCCATCGGCATCAACATCACCCGGTTTCAGACACT |
| | | GCTGGCCCTGCACAGAAGCTACCTGACACCTGGCGATAGC |
| | | AGCAGCGGATGGACAGCTGGTGCCGCCGCTTACTATGTGG |
| | | GCTACCTGCAGCCTAGAACCTTCCTGCTGAAGTACAACGA |
| | | GAACGGCACCATCACCGACGCCGTGGATTGTGCTCTGGAT |
| | | CCTCTGAGCGAGACAAAGTGCACCCTGAAGTCCTTCACCG |
| | | TGGAAAAGGGCATCTACCAGACCAGCAACTTCCGGGTGCA |
| | | GCCCACCGAATCCATCGTGCGGTTCCCCAATATCACCAAT |
| | | CTGTGCCCCTTCGACGAGGTGTTCAATGCCACCACCTTCG |
| | | CCTCTGTGTACGCCTGGAACCGGAAGCGGATCAGCAATTG |
| | | CGTGGCCGACTACTCCGTGCTGTACAACTTCGCCCCCTTC |
| | | TTCGCATTCAAGTGCTACGGCGTGTCCCCTACCAAGCTGA |
| | | ACGACCTGTGCTTCACAAACGTGTACGCCGACAGCTTCGT |
| | | GATCCGGGGAAACGAAGTGTCACAGATTGCCCCTGGACAG |
| | | ACAGGCAACATCGCCGACTACAACTACAAGCTGCCCGACG |
| | | ACTTCACCGGCTGTGTGATTGCCTGGAACAGCAACAAGCT |
| | | GGACTCCACCGTCGGCGGCAACTACAATTACAGGTACCGG |
| | | CTGTTCCGGAAGTCCAAGCTGAAGCCCTTCGAGCGGGACA |
| | | TCTCCACCGAGATCTATCAGGCCGGCAACAAGCCTTGTAA |
| | | CGGCGTGGCAGGCGTGAACTGCTACTTCCCACTGCAGTCC |
| | | TACGGCTTTAGGCCCACATACGGCGTGGGCCACCAGCCCT |
| | | ACAGAGTGGTGGTGCTGAGCTTCGAACTGCTGCATGCCCC |
| | | TGCCACAGTGTGCGGCCCTAAGAAAAGCACCAATCTCGTG |
| | | AAGAACAAATGCGTGAACTTCAACTTCAACGGCCTGACCG |
| | | GCACCGGCGTGCTGACAGAGAGCAACAAGAAGTTCCTGCC |
| | | ATTCCAGCAGTTTGGCCGGGATATCGCCGATACCACAGAC |
| | | GCCGTTAGAGATCCCCAGACACTGGAAATCCTGGACATCA |
| | | CCCCTTGCAGCTTCGGCGGAGTGTCTGTGATCACCCCTGG |
| | | CACCAACACCAGCAATCAGGTGGCAGTGCTGTACCAGGGC |
| | | GTGAACTGTACCGAAGTGCCCGTGGCCATTCACGCCGATC |
| | | AGCTGACACCTACATGGCGGGTGTACTCCACCGGCAGCAA |
| | | TGTGTTTCAGACCAGAGCCGGCTGTCTGATCGGAGCCGAG |
| | | TACGTGAACAATAGCTACGAGTGCGACATCCCCATCGGCG |
| | | CTGGAATCTGCGCCAGCTACCAGACACAGACAAAGAGCCA |
| | | CCGGAGAGCCAGAAGCGTGGCCAGCCAGAGCATCATTGCC |
| | | TACACAATGTCTCTGGGCGCCGAGAACAGCGTGGCCTACT |
| | | CCAACAACTCTATCGCTATCCCCACCAACTTCACCATCAG |
| | | CGTGACCACAGAGATCCTGCCTGTGTCCATGACCAAGACC |
| | | AGCGTGGACTGCACCATGTACATCTGCGGCGATTCCACCG |
| | | AGTGCTCCAACCTGCTGCTGCAGTACGGCAGCTTCTGCAC |
| | | CCAGCTGAAAAGAGCCCTGACAGGGATCGCCGTGGAACAG |
| | | GACAAGAACACCCAAGAGGTGTTCGCCCAAGTGAAGCAGA |
| | | TCTACAAGACCCCTCCTATCAAGTACTTCGGCGGCTTCAA |
| | | TTTCAGCCAGATTCTGCCCGATCCTAGCAAGCCCAGCAAG |
| | | CGGAGCTTCATCGAGGACCTGCTGTTCAACAAAGTGACAC |
| | | TGGCCGACGCCGGCTTCATCAAGCAGTATGGCGATTGTCT |
| | | GGGCGACATTGCCGCCAGGGATCTGATTTGCGCCCAGAAG |
| | | TTTAACGGACTGACAGTGCTGCCTCCTCTGCTGACCGATG |
| | | AGATGATCGCCCAGTACACATCTGCCCTGCTGGCCGGCAC |
| | | AATCACAAGCGGCTGGACATTTGGAGCAGGCGCCGCTCTG |
| | | CAGATCCCCTTTGCTATGCAGATGGCCTACCGGTTCAACG |
| | | GCATCGGAGTGACCCAGAATGTGCTGTACGAGAACCAGAA |
| | | GCTGATCGCCAACCAGTTCAACAGCGCCATCGGCAAGATC |
| | | CAGGACAGCCTGAGCAGCACAGCAAGCGCCCTGGGAAAGC |
| | | TGCAGGACGTGGTCAACCACAATGCCCAGGCACTGAACAC |
| | | CCTGGTCAAGCAGCTGTCCTCCAAGTTCGGCGCCATCAGC |
| | | TCTGTGCTGAACGATATCCTGAGCAGACTGGACCCTCCTG |
| | | AGGCCGAGGTGCAGATCGACAGACTGATCACAGGCAGACT |
| | | GCAGAGCCTCCAGACATACGTGACCCAGCAGCTGATCAGA |
| | | GCCGCCGAGATTAGAGCCTCTGCCAATCTGGCCGCCACCA |
| | | AGATGTCTGAGTGTGTGCTGGGCCAGAGCAAGAGAGTGGA |
| | | CTTTTGCGGCAAGGGCTACCACCTGATGAGCTTCCCTCAG |
| | | TCTGCCCCTCACGGCGTGGTGTTTCTGCACGTGACATATG |
| | | TGCCCGCTCAAGAGAAGAATTTCACCACCGCTCCAGCCAT |
| | | CTGCCACGACGGCAAAGCCCACTTTCCTAGAGAAGGCGTG |
| | | TTCGTGTCCAACGGCACCCATTGGTTCGTGACACAGCGGA |
| | | ACTTCTACGAGCCCCAGATCATCACCACCGACAACACCTT |
| | | CGTGTCTGGCAACTGCGACGTCGTGATCGGCATTGTGAAC |
| | | AATACCGTGTACGACCCTCTGCAGCCCGAGCTGGACAGCT |
| | | TCAAAGAGGAACTGGACAAGTACTTTAAGAACCACACAAG |
| | | CCCCGACGTGGACCTGGGCGATATCAGCGGAATCAATGCC |
| | | AGCGTCGTGAACATCCAGAAAGAGATCGACCGGCTGAACG |
| | | AGGTGGCCAAGAATCTGAACGAGAGCCTGATCGACCTGCA |
| | | AGAACTGGGGAAGTACGAGCAGTACATCAAGTGGCCCTGG |
| | | TACATCTGGCTGGGCTTTATCGCCGGACTGATTGCCATCG |
| | | TGATGGTCACAATCATGCTGTGTTGCATGACCAGCTGCTG |
| | | TAGCTGCCTGAAGGGCTGTTGTAGCTGTGGCAGCTGCTGC |
| | | AAGTTCGACGAGGACGATTCTGAGCCCGTGCTGAAGGGCG |
| | | TGAAACTGCACTACACATGATGActcgagctggtactgca |
| | | tgcacgcaatgctagctgcccctttcccgtcctgggtacc |
| | | ccgagtctcccccgacctcgggtcccaggtatgctcccac |
| | | ctccacctgccccactcaccacctctgctagttccagaca |
| | | cctcccaagcacgcagcaatgcagctcaaaacgcttagcc |
| | | tagccacacccccacgggaaacagcagtgattaaccttta |
| | | gcaataaacgaaagtttaactaagctatactaaccccagg |
| | | gttggtcaatttcgtgccagccacaccctggagctagcaa |
| | | aaaaaaaaaaaaaaaaaaaaaaaaaaaagcatatgactaa |
| | | aaaaaaaaaaaaaaaaaaaaaaaaaaaaaaaaaaaaaaaa |
| | | aaaaaaaaaaaaaaaaaaaaaaaaaaa |

TABLE 16A
Sequence of one embodiment of an exemplary Omicron XBB.1.5-specific
RNA vaccine

| SEQ | | |
|---|---|---|
| ID | | |
| NO. | Brief Description | Sequence |
| 129 | Amino acid sequence of RNA-encoded | MFVFLVLLPLVSSQCVNLITRTQSYTNSFTRGVYYPDKVF |
| | SARS-CoV-2 S protein from an | RSSVLHSTQDLFLPFFSNVTWFHAIHVSGTNGTKRFDNPA |
| | Omicron XBB.1.5 variant (with PRO | LPFNDGVYFASTEKSNIIRGWIFGTTLDSKTQSLLIVNNA |
| | mutations at positions corresponding | TNVVIKVCEFQFCNDPFLDVYQKNNKSWMESEFRVYSSAN |
| | to K986P and V987P of SEQ ID NO: 1; | NCTFEYVSQPFLMDLEGKEGNFKNLREFVFKNIDGYFKIY |
| | i.e., PRO mutations at positions 982 | SKHTPINLERDLPQGFSALEPLVDLPIGINITRFQTLLAL |
| | and 983 of SEQ ID NO: 129) | HRSYLTPVDSSSGWTAGAAAYYVGYLQPRTFLLKYNENGT |
| | | ITDAVDCALDPLSETKCTLKSFTVEKGIYQTSNFRVQPTE |
| | | SIVRFPNITNLCPFHEVFNATTFASVYAWNRKRISNCVAD |
| | | YSVIYNFAPFFAFKCYGVSPTKLNDLCFTNVYADSFVIRG |
| | | NEVSQIAPGQTGNIADYNYKLPDDFTGCVIAWNSNKLDSK |
| | | PSGNYNYLYRLFRKSKLKPFERDISTEIYQAGNKPCNGVA |
| | | GPNCYSPLQSYGFRPTYGVGHQPYRVVVLSFELLHAPATV |
| | | CGPKKSTNLVKNKCVNFNFNGLTGTGVLTESNKKFLPFQQ |
| | | FGRDIADTTDAVRDPQTLEILDITPCSFGGVSVITPGTNT |
| | | SNQVAVLYQGVNCTEVPVAIHADQLTPTWRVYSTGSNVFQ |
| | | TRAGCLIGAEYVNNSYECDIPIGAGICASYQTQTKSHRRA |
| | | RSVASQSIIAYTMSLGAENSVAYSNNSIAIPTNFTISVTT |
| | | EILPVSMTKTSVDCTMYICGDSTECSNLLLQYGSFCTQLK |
| | | RALTGIAVEQDKNTQEVFAQVKQIYKTPPIKYFGGFNFSQ |
| | | ILPDPSKPSKRSFIEDLLFNKVTLADAGFIKQYGDCLGDI |
| | | AARDLICAQKFNGLTVLPPLLTDEMIAQYTSALLAGTITS |
| | | GWTFGAGAALQIPFAMQMAYRFNGIGVTQNVLYENQKLIA |
| | | NQFNSAIGKIQDSLSSTASALGKLQDVVNHNAQALNTLVK |
| | | QLSSKFGAISSVLNDILSRLDPPEAEVQIDRLITGRLQSL |
| | | QTYVTQQLIRAAEIRASANLAATKMSECVLGQSKRVDFCG |
| | | KGYHLMSFPQSAPHGVVFLHVTYVPAQEKNFTTAPAICHD |
| | | GKAHFPREGVFVSNGTHWFVTQRNFYEPQIITTDNTFVSG |
| | | NCDVVIGIVNNTVYDPLQPELDSFKEELDKYFKNHTSPDV |
| | | DLGDISGINASVVNIQKEIDRLNEVAKNLNESLIDLQELG |
| | | KYEQYIKWPWYIWLGFIAGLIAIVMVTIMLCCMTSCCSCL |
| | | KGCCSCGSCCKFDEDDSEPVLKGVKLHYT |
| 130 | RNA sequence encoding a SARS-CoV-2 | auguucguguuccuggugcugcugccucugguguccagcc |
| | S protein from an Omicron XBB.1.5 | agugugugaaccugaucaccagaacacagucauacaccaa |
| | variant | cagcuuuaccagaggcguguacuaccccgacaagguguuc |
| | | agauccagcgugcugcacucuacccaggaccuguuccugc |
| | | cuuucuucagcaacgugaccugguuccacgccauccacgu |
| | | guccggcaccaauggcaccaagagauucgacaaccccgcc |
| | | cugcccuucaacgacgggguguacuuugccagcaccgaga |
| | | aguccaacaucaucagaggcuggaucuucggcaccacacu |
| | | ggacagcaagacccagagccugcugaucgugaacaacgcc |
| | | accaacguggucaucaaagugugcgaguuccaguucugca |
| | | acgaccccuuccuggacgucuaccagaagaacaacaagag |
| | | cuggauggaaagcgaguuccggguguacagcagcgccaac |
| | | aacugcaccuucgaguacgugucccagccuuuccugaugg |
| | | accuggaaggcaaggagggcaacuucaagaaccugcgcga |
| | | guucguguuuaagaacaucgacggcuacuucaagaucuac |
| | | agcaagcacaccccuaucaaccucgagcgggaucugccuc |
| | | agggcuucucugcucuggaaccccugguggaucugcccau |
| | | cggcaucaacaucacccgguuucagacacugcuggcccug |
| | | cacagaagcuaccugacaccuguggauagcagcagcggau |
| | | ggacagcuggugccgccgcuuacuaugugggcuaccugca |
| | | gccuagaaccuuccugcugaaguacaacgagaacggcacc |
| | | aucaccgacgccguggauugugcucuggauccucugagcg |
| | | agacaaagugcacccugaaguccuucaccguggaaaaggg |
| | | caucuaccagaccagcaacuuccgggugcagcccaccgaa |
| | | uccaucgugcgguuccccaauaucaccaaucugugccccu |
| | | uccacgagguguucaaugccaccaccuucgccucugugua |
| | | cgccuggaaccggaagcggaucagcaauugcguggccgac |
| | | uacuccgugaucuacaacuucgcccccuucuucgcauuca |
| | | agugcuacggcguguccccuaccaagcugaacgaccugug |
| | | cuucacaaacguguacgccgacagcuucgugauccgggga |
| | | aacgaagugucacagauugccccuggacagacaggcaaca |
| | | ucgccgacuacaacuacaagcugcccgacgacuucaccgg |
| | | cugugugauugccuggaacagcaacaagcuggacuccaaa |
| | | cccagcggcaacuacaauuaccuguaccggcuguuccgga |
| | | aguccaagcugaagcccuucgagcgggacaucuccaccga |
| | | gaucuaucaggccggcaacaagccuuguaacggcguggca |
| | | ggccccaacugcuacagcccacugcaguccuacggcuuua |
| | | ggcccacauacggcgugggccaccagcccuacagaguggu |
| | | ggugcugagcuucgaacugcugcaugccccugccacagug |
| | | ugcggcccuaagaaaagcaccaaucucgugaagaacaaau |
| | | gcgugaacuucaacuucaacggccugaccggcaccggcgu |
| | | gcugacagagagcaacaagaaguuccugccauuccagcag |
| | | uuuggccgggauaucgccgauaccacagacgccguuagag |
| | | auccccagacacuggaaauccuggacaucaccccuugcag |
| | | cuucggcggagugucugugaucaccccuggcaccaacacc |
| | | agcaaucagguggcagugcuguaccagggcgugaacugua |
| | | ccgaagugcccguggccauucacgccgaucagcugacacc |
| | | uacauggcggguguacuccaccggcagcaauguguuucag |
| | | accagagccggcugucugaucggagccgaguacgugaaca |
| | | auagcuacgagugcgacauccccaucggcgcuggaaucug |
| | | cgccagcuaccagacacagacaaagagccaccggagagcc |
| | | agaagcguggccagccagagcaucauugccuacacaaugu |
| | | cucugggcgccgagaacagcguggccuacuccaacaacuc |
| | | uaucgcuauccccaccaacuucaccaucagcgugaccaca |
| | | gagauccugccuguguccaugaccaagaccagcguggacu |
| | | gcaccauguacaucugcggcgauuccaccgagugcuccaa |
| | | ccugcugcugcaguacggcagcuucugcacccagcugaaa |
| | | agagcccugacagggaucgccguggaacaggacaagaaca |
| | | cccaagagguguucgcccaagugaagcagaucuacaagac |
| | | cccuccuaucaaguacuucggcggcuucaauuucagccag |
| | | auucugcccgauccuagcaagcccagcaagcggagcuuca |
| | | ucgaggaccugcuguucaacaaagugacacuggccgacgc |
| | | cggcuucaucaagcaguauggcgauugucugggcgacauu |
| | | gccgccagggaucugauuugcgcccagaaguuuaacggac |
| | | ugacagugcugccuccucugcugaccgaugagaugaucgc |
| | | ccaguacacaucugcccugcuggccggcacaaucacaagc |
| | | ggcuggacauuuggagcaggcgccgcucugcagauccccu |
| | | uugcuaugcagauggccuaccgguucaacggcaucggagu |
| | | gacccagaaugugcuguacgagaaccagaagcugaucgcc |
| | | aaccaguucaacagcgccaucggcaagauccaggacagcc |
| | | ugagcagcacagcaagcgcccugggaaagcugcaggacgu |
| | | ggucaaccacaaugcccaggcacugaacacccuggucaag |
| | | cagcuguccuccaaguucggcgccaucagcucugugcuga |
| | | acgauauccugagcagacuggacccuccugaggccgaggu |
| | | gcagaucgacagacugaucacaggcagacugcagagccuc |
| | | cagacauacgugacccagcagcugaucagagccgccgaga |
| | | uuagagccucugccaaucuggccgccaccaagaugucuga |
| | | gugugugcugggccagagcaagagaguggacuuuugcggc |
| | | aagggcuaccaccugaugagcuucccucagucugccccuc |
| | | acggcgugguguuucugcacgugacauaugugcccgcuca |
| | | agagaagaauuucaccaccgcuccagccaucugccacgac |
| | | ggcaaagcccacuuuccuagagaaggcguguucgugucca |
| | | acggcacccauugguucgugacacagcggaacuucuacga |
| | | gccccagaucaucaccaccgacaacaccuucgugucuggc |
| | | aacugcgacgucgugaucggcauugugaacaauaccgugu |
| | | acgacccucugcagcccgagcuggacagcuucaaagagga |
| | | acuggacaaguacuuuaagaaccacacaagccccgacgug |
| | | gaccugggcgauaucagcggaaucaaugccagcgucguga |
| | | acauccagaaagagaucgaccggcugaacgagguggccaa |
| | | gaaucugaacgagagccugaucgaccugcaagaacugggg |
| | | aaguacgagcaguacaucaaguggcccugguacaucuggc |
| | | ugggcuuuaucgccggacugauugccaucgugauggucac |
| | | aaucaugcuguguugcaugaccagcugcuguagcugccug |
| | | aagggcuguuguagcuguggcagcugcugcaaguucgacg |
| | | aggacgauucugagcccgugcugaagggcgugaaacugca |
| | | cuacacaugauga |
| 131 | DNA sequence encoding a SARS-CoV-2 | atgttcgtgttcctggtgctgctgcctctggtgtccagcc |
| | S protein from an Omicron XBB.1.5 | agtgtgtgaacctgatcaccagaacacagtcatacaccaa |
| | variant | cagctttaccagaggcgtgtactaccccgacaaggtgttc |
| | | agatccagcgtgctgcactctacccaggacctgttcctgc |
| | | ctttcttcagcaacgtgacctggttccacgccatccacgt |
| | | gtccggcaccaatggcaccaagagattcgacaaccccgcc |
| | | ctgcccttcaacgacggggtgtactttgccagcaccgaga |
| | | agtccaacatcatcagaggctggatcttcggcaccacact |
| | | ggacagcaagacccagagcctgctgatcgtgaacaacgcc |
| | | accaacgtggtcatcaaagtgtgcgagttccagttctgca |
| | | acgaccccttcctggacgtctaccagaagaacaacaagag |
| | | ctggatggaaagcgagttccgggtgtacagcagcgccaac |
| | | aactgcaccttcgagtacgtgtcccagcctttcctgatgg |
| | | acctggaaggcaaggagggcaacttcaagaacctgcgcga |
| | | gttcgtgtttaagaacatcgacggctacttcaagatctac |
| | | agcaagcacacccctatcaacctcgagcgggatctgcctc |
| | | agggcttctctgctctggaacccctggtggatctgcccat |
| | | cggcatcaacatcacccggtttcagacactgctggccctg |
| | | cacagaagctacctgacacctgtggatagcagcagcggat |
| | | ggacagctggtgccgccgcttactatgtgggctacctgca |
| | | gcctagaaccttcctgctgaagtacaacgagaacggcacc |
| | | atcaccgacgccgtggattgtgctctggatcctctgagcg |
| | | agacaaagtgcaccctgaagtccttcaccgtggaaaaggg |
| | | catctaccagaccagcaacttccgggtgcagcccaccgaa |
| | | tccatcgtgcggttccccaatatcaccaatctgtgcccct |
| | | tccacgaggtgttcaatgccaccaccttcgcctctgtgta |
| | | cgcctggaaccggaagcggatcagcaattgcgtggccgac |
| | | tactccgtgatctacaacttcgcccccttcttcgcattca |
| | | agtgctacggcgtgtcccctaccaagctgaacgacctgtg |
| | | cttcacaaacgtgtacgccgacagcttcgtgatccgggga |
| | | aacgaagtgtcacagattgcccctggacagacaggcaaca |
| | | tcgccgactacaactacaagctgcccgacgacttcaccgg |
| | | ctgtgtgattgcctggaacagcaacaagctggactccaaa |
| | | cccagcggcaactacaattacctgtaccggctgttccgga |
| | | agtccaagctgaagcccttcgagcgggacatctccaccga |
| | | gatctatcaggccggcaacaagccttgtaacggcgtggca |
| | | ggccccaactgctacagcccactgcagtcctacggcttta |
| | | ggcccacatacggcgtgggccaccagccctacagagtggt |
| | | ggtgctgagcttcgaactgctgcatgcccctgccacagtg |
| | | tgcggccctaagaaaagcaccaatctcgtgaagaacaaat |
| | | gcgtgaacttcaacttcaacggcctgaccggcaccggcgt |
| | | gctgacagagagcaacaagaagttcctgccattccagcag |
| | | tttggccgggatatcgccgataccacagacgccgttagag |
| | | atccccagacactggaaatcctggacatcaccccttgcag |
| | | cttcggcggagtgtctgtgatcacccctggcaccaacacc |
| | | agcaatcaggtggcagtgctgtaccagggcgtgaactgta |
| | | ccgaagtgcccgtggccattcacgccgatcagctgacacc |
| | | tacatggcgggtgtactccaccggcagcaatgtgtttcag |
| | | accagagccggctgtctgatcggagccgagtacgtgaaca |
| | | atagctacgagtgcgacatccccatcggcgctggaatctg |
| | | cgccagctaccagacacagacaaagagccaccggagagcc |
| | | agaagcgtggccagccagagcatcattgcctacacaatgt |
| | | ctctgggcgccgagaacagcgtggcctactccaacaactc |
| | | tatcgctatccccaccaacttcaccatcagcgtgaccaca |
| | | gagatcctgcctgtgtccatgaccaagaccagcgtggact |
| | | gcaccatgtacatctgcggcgattccaccgagtgctccaa |
| | | cctgctgctgcagtacggcagcttctgcacccagctgaaa |
| | | agagccctgacagggatcgccgtggaacaggacaagaaca |
| | | cccaagaggtgttcgcccaagtgaagcagatctacaagac |
| | | ccctcctatcaagtacttcggcggcttcaatttcagccag |
| | | attctgcccgatcctagcaagcccagcaagcggagcttca |
| | | tcgaggacctgctgttcaacaaagtgacactggccgacgc |
| | | cggcttcatcaagcagtatggcgattgtctgggcgacatt |
| | | gccgccagggatctgatttgcgcccagaagtttaacggac |
| | | tgacagtgctgcctcctctgctgaccgatgagatgatcgc |
| | | ccagtacacatctgccctgctggccggcacaatcacaagc |
| | | ggctggacatttggagcaggcgccgctctgcagatcccct |
| | | ttgctatgcagatggcctaccggttcaacggcatcggagt |
| | | gacccagaatgtgctgtacgagaaccagaagctgatcgcc |
| | | aaccagttcaacagcgccatcggcaagatccaggacagcc |
| | | tgagcagcacagcaagcgccctgggaaagctgcaggacgt |
| | | ggtcaaccacaatgcccaggcactgaacaccctggtcaag |
| | | cagctgtcctccaagttcggcgccatcagctctgtgctga |
| | | acgatatcctgagcagactggaccctcctgaggccgaggt |
| | | gcagatcgacagactgatcacaggcagactgcagagcctc |
| | | cagacatacgtgacccagcagctgatcagagccgccgaga |
| | | ttagagcctctgccaatctggccgccaccaagatgtctga |
| | | gtgtgtgctgggccagagcaagagagtggacttttgcggc |
| | | aagggctaccacctgatgagcttccctcagtctgcccctc |
| | | acggcgtggtgtttctgcacgtgacatatgtgcccgctca |
| | | agagaagaatttcaccaccgctccagccatctgccacgac |
| | | ggcaaagcccactttcctagagaaggcgtgttcgtgtcca |
| | | acggcacccattggttcgtgacacagcggaacttctacga |
| | | gccccagatcatcaccaccgacaacaccttcgtgtctggc |
| | | aactgcgacgtcgtgatcggcattgtgaacaataccgtgt |
| | | acgaccctctgcagcccgagctggacagcttcaaagagga |
| | | actggacaagtactttaagaaccacacaagccccgacgtg |
| | | gacctgggcgatatcagcggaatcaatgccagcgtcgtga |
| | | acatccagaaagagatcgaccggctgaacgaggtggccaa |
| | | gaatctgaacgagagcctgatcgacctgcaagaactgggg |
| | | aagtacgagcagtacatcaagtggccctggtacatctggc |
| | | tgggctttatcgccggactgattgccatcgtgatggtcac |
| | | aatcatgctgtgttgcatgaccagctgctgtagctgcctg |
| | | aagggctgttgtagctgtggcagctgctgcaagttcgacg |
| | | aggacgattctgagcccgtgctgaagggcgtgaaactgca |
| | | ctacacatgatga |
| 132 | Full length RNA construct encoding a | Agaauaaacuaguauucuucugguccccacagacucagag |
| | SARS-CoV-2 S protein from an | agaacccgccaccauguucguguuccuggugcugcugccu |
| | Omicron XBB.1.5 variant | cugguguccagccagugugugaaccugaucaccagaacac |
| | | agucauacaccaacagcuuuaccagaggcguguacuaccc |
| | | cgacaagguguucagauccagcgugcugcacucuacccag |
| | | gaccuguuccugccuuucuucagcaacgugaccugguucc |
| | | acgccauccacguguccggcaccaauggcaccaagagauu |
| | | cgacaaccccgcccugcccuucaacgacgggguguacuuu |
| | | gccagcaccgagaaguccaacaucaucagaggcuggaucu |
| | | ucggcaccacacuggacagcaagacccagagccugcugau |
| | | cgugaacaacgccaccaacguggucaucaaagugugcgag |
| | | uuccaguucugcaacgaccccuuccuggacgucuaccaga |
| | | agaacaacaagagcuggauggaaagcgaguuccgggugua |
| | | cagcagcgccaacaacugcaccuucgaguacgugucccag |
| | | ccuuuccugauggaccuggaaggcaaggagggcaacuuca |
| | | agaaccugcgcgaguucguguuuaagaacaucgacggcua |
| | | cuucaagaucuacagcaagcacaccccuaucaaccucgag |
| | | cgggaucugccucagggcuucucugcucuggaaccccugg |
| | | uggaucugcccaucggcaucaacaucacccgguuucagac |
| | | acugcuggcccugcacagaagcuaccugacaccuguggau |
| | | agcagcagcggauggacagcuggugccgccgcuuacuaug |
| | | ugggcuaccugcagccuagaaccuuccugcugaaguacaa |
| | | cgagaacggcaccaucaccgacgccguggauugugcucug |
| | | gauccucugagcgagacaaagugcacccugaaguccuuca |
| | | ccguggaaaagggcaucuaccagaccagcaacuuccgggu |
| | | gcagcccaccgaauccaucgugcgguuccccaauaucacc |
| | | aaucugugccccuuccacgagguguucaaugccaccaccu |
| | | ucgccucuguguacgccuggaaccggaagcggaucagcaa |
| | | uugcguggccgacuacuccgugaucuacaacuucgccccc |
| | | uucuucgcauucaagugcuacggcguguccccuaccaagc |
| | | ugaacgaccugugcuucacaaacguguacgccgacagcuu |
| | | cgugauccggggaaacgaagugucacagauugccccugga |
| | | cagacaggcaacaucgccgacuacaacuacaagcugcccg |
| | | acgacuucaccggcugugugauugccuggaacagcaacaa |
| | | gcuggacuccaaacccagcggcaacuacaauuaccuguac |
| | | cggcuguuccggaaguccaagcugaagcccuucgagcggg |
| | | acaucuccaccgagaucuaucaggccggcaacaagccuug |
| | | uaacggcguggcaggccccaacugcuacagcccacugcag |
| | | uccuacggcuuuaggcccacauacggcgugggccaccagc |
| | | ccuacagagugguggugcugagcuucgaacugcugcaugc |
| | | cccugccacagugugcggcccuaagaaaagcaccaaucuc |
| | | gugaagaacaaaugcgugaacuucaacuucaacggccuga |
| | | ccggcaccggcgugcugacagagagcaacaagaaguuccu |
| | | gccauuccagcaguuuggccgggauaucgccgauaccaca |
| | | gacgccguuagagauccccagacacuggaaauccuggaca |
| | | ucaccccuugcagcuucggcggagugucugugaucacccc |
| | | uggcaccaacaccagcaaucagguggcagugcuguaccag |
| | | ggcgugaacuguaccgaagugcccguggccauucacgccg |
| | | aucagcugacaccuacauggcggguguacuccaccggcag |
| | | caauguguuucagaccagagccggcugucugaucggagcc |
| | | gaguacgugaacaauagcuacgagugcgacauccccaucg |
| | | gcgcuggaaucugcgccagcuaccagacacagacaaagag |
| | | ccaccggagagccagaagcguggccagccagagcaucauu |
| | | gccuacacaaugucucugggcgccgagaacagcguggccu |
| | | acuccaacaacucuaucgcuauccccaccaacuucaccau |
| | | cagcgugaccacagagauccugccuguguccaugaccaag |
| | | accagcguggacugcaccauguacaucugcggcgauucca |
| | | ccgagugcuccaaccugcugcugcaguacggcagcuucug |
| | | cacccagcugaaaagagcccugacagggaucgccguggaa |
| | | caggacaagaacacccaagagguguucgcccaagugaagc |
| | | agaucuacaagaccccuccuaucaaguacuucggcggcuu |
| | | caauuucagccagauucugcccgauccuagcaagcccagc |
| | | aagcggagcuucaucgaggaccugcuguucaacaaaguga |
| | | cacuggccgacgccggcuucaucaagcaguauggcgauug |
| | | ucugggcgacauugccgccagggaucugauuugcgcccag |
| | | aaguuuaacggacugacagugcugccuccucugcugaccg |
| | | augagaugaucgcccaguacacaucugcccugcuggccgg |
| | | cacaaucacaagcggcuggacauuuggagcaggcgccgcu |
| | | cugcagauccccuuugcuaugcagauggccuaccgguuca |
| | | acggcaucggagugacccagaaugugcuguacgagaacca |
| | | gaagcugaucgccaaccaguucaacagcgccaucggcaag |
| | | auccaggacagccugagcagcacagcaagcgcccugggaa |
| | | agcugcaggacguggucaaccacaaugcccaggcacugaa |
| | | cacccuggucaagcagcuguccuccaaguucggcgccauc |
| | | agcucugugcugaacgauauccugagcagacuggacccuc |
| | | cugaggccgaggugcagaucgacagacugaucacaggcag |
| | | acugcagagccuccagacauacgugacccagcagcugauc |
| | | agagccgccgagauuagagccucugccaaucuggccgcca |
| | | ccaagaugucugagugugugcugggccagagcaagagagu |
| | | ggacuuuugcggcaagggcuaccaccugaugagcuucccu |
| | | cagucugccccucacggcgugguguuucugcacgugacau |
| | | augugcccgcucaagagaagaauuucaccaccgcuccagc |
| | | caucugccacgacggcaaagcccacuuuccuagagaaggc |
| | | guguucguguccaacggcacccauugguucgugacacagc |
| | | ggaacuucuacgagccccagaucaucaccaccgacaacac |
| | | cuucgugucuggcaacugcgacgucgugaucggcauugug |
| | | aacaauaccguguacgacccucugcagcccgagcuggaca |
| | | gcuucaaagaggaacuggacaaguacuuuaagaaccacac |
| | | aagccccgacguggaccugggcgauaucagcggaaucaau |
| | | gccagcgucgugaacauccagaaagagaucgaccggcuga |
| | | acgagguggccaagaaucugaacgagagccugaucgaccu |
| | | gcaagaacuggggaaguacgagcaguacaucaaguggccc |
| | | ugguacaucuggcugggcuuuaucgccggacugauugcca |
| | | ucgugauggucacaaucaugcuguguugcaugaccagcug |
| | | cuguagcugccugaagggcuguuguagcuguggcagcugc |
| | | ugcaaguucgacgaggacgauucugagcccgugcugaagg |
| | | gcgugaaacugcacuacacaugaugacucgagcugguacu |
| | | gcaugcacgcaaugcuagcugccccuuucccguccugggu |
| | | accccgagucucccccgaccucgggucccagguaugcucc |
| | | caccuccaccugccccacucaccaccucugcuaguuccag |
| | | acaccucccaagcacgcagcaaugcagcucaaaacgcuua |
| | | gccuagccacacccccacgggaaacagcagugauuaaccu |
| | | uuagcaauaaacgaaaguuuaacuaagcuauacuaacccc |
| | | aggguuggucaauuucgugccagccacacccuggagcuag |
| | | caaaaaaaaaaaaaaaaaaaaaaaaaaaaaagcauaugac |
| | | uaaaaaaaaaaaaaaaaaaaaaaaaaaaaaaaaaaaaaaa |
| | | aaaaaaaaaaaaaaaaaaaaaaaaaaaaaa |
| 133 | Full length DNA construct encoding a | Agaataaactagtattcttctggtccccacagactcagag |
| | SARS-CoV-2 S protein from an | agaacccgccaccatgttcgtgttcctggtgctgctgcct |
| | Omicron XBB.1.5 variant | ctggtgtccagccagtgtgtgaacctgatcaccagaacac |
| | | agtcatacaccaacagctttaccagaggcgtgtactaccc |
| | | cgacaaggtgttcagatccagcgtgctgcactctacccag |
| | | gacctgttcctgcctttcttcagcaacgtgacctggttcc |
| | | acgccatccacgtgtccggcaccaatggcaccaagagatt |
| | | cgacaaccccgccctgcccttcaacgacggggtgtacttt |
| | | gccagcaccgagaagtccaacatcatcagaggctggatct |
| | | tcggcaccacactggacagcaagacccagagcctgctgat |
| | | cgtgaacaacgccaccaacgtggtcatcaaagtgtgcgag |
| | | ttccagttctgcaacgaccccttcctggacgtctaccaga |
| | | agaacaacaagagctggatggaaagcgagttccgggtgta |
| | | cagcagcgccaacaactgcaccttcgagtacgtgtcccag |
| | | cctttcctgatggacctggaaggcaaggagggcaacttca |
| | | agaacctgcgcgagttcgtgtttaagaacatcgacggcta |
| | | cttcaagatctacagcaagcacacccctatcaacctcgag |
| | | cgggatctgcctcagggcttctctgctctggaacccctgg |
| | | tggatctgcccatcggcatcaacatcacccggtttcagac |
| | | actgctggccctgcacagaagctacctgacacctgtggat |
| | | agcagcagcggatggacagctggtgccgccgcttactatg |
| | | tgggctacctgcagcctagaaccttcctgctgaagtacaa |
| | | cgagaacggcaccatcaccgacgccgtggattgtgctctg |
| | | gatcctctgagcgagacaaagtgcaccctgaagtccttca |
| | | ccgtggaaaagggcatctaccagaccagcaacttccgggt |
| | | gcagcccaccgaatccatcgtgcggttccccaatatcacc |
| | | aatctgtgccccttccacgaggtgttcaatgccaccacct |
| | | tcgcctctgtgtacgcctggaaccggaagcggatcagcaa |
| | | ttgcgtggccgactactccgtgatctacaacttcgccccc |
| | | ttcttcgcattcaagtgctacggcgtgtcccctaccaagc |
| | | tgaacgacctgtgcttcacaaacgtgtacgccgacagctt |
| | | cgtgatccggggaaacgaagtgtcacagattgcccctgga |
| | | cagacaggcaacatcgccgactacaactacaagctgcccg |
| | | acgacttcaccggctgtgtgattgcctggaacagcaacaa |
| | | gctggactccaaacccagcggcaactacaattacctgtac |
| | | cggctgttccggaagtccaagctgaagcccttcgagcggg |
| | | acatctccaccgagatctatcaggccggcaacaagccttg |
| | | taacggcgtggcaggccccaactgctacagcccactgcag |
| | | tcctacggctttaggcccacatacggcgtgggccaccagc |
| | | cctacagagtggtggtgctgagcttcgaactgctgcatgc |
| | | ccctgccacagtgtgcggccctaagaaaagcaccaatctc |
| | | gtgaagaacaaatgcgtgaacttcaacttcaacggcctga |
| | | ccggcaccggcgtgctgacagagagcaacaagaagttcct |
| | | gccattccagcagtttggccgggatatcgccgataccaca |
| | | gacgccgttagagatccccagacactggaaatcctggaca |
| | | tcaccccttgcagcttcggcggagtgtctgtgatcacccc |
| | | tggcaccaacaccagcaatcaggtggcagtgctgtaccag |
| | | ggcgtgaactgtaccgaagtgcccgtggccattcacgccg |
| | | atcagctgacacctacatggcgggtgtactccaccggcag |
| | | caatgtgtttcagaccagagccggctgtctgatcggagcc |
| | | gagtacgtgaacaatagctacgagtgcgacatccccatcg |
| | | gcgctggaatctgcgccagctaccagacacagacaaagag |
| | | ccaccggagagccagaagcgtggccagccagagcatcatt |
| | | gcctacacaatgtctctgggcgccgagaacagcgtggcct |
| | | actccaacaactctatcgctatccccaccaacttcaccat |
| | | cagcgtgaccacagagatcctgcctgtgtccatgaccaag |
| | | accagcgtggactgcaccatgtacatctgcggcgattcca |
| | | ccgagtgctccaacctgctgctgcagtacggcagcttctg |
| | | cacccagctgaaaagagccctgacagggatcgccgtggaa |
| | | caggacaagaacacccaagaggtgttcgcccaagtgaagc |
| | | agatctacaagacccctcctatcaagtacttcggcggctt |
| | | caatttcagccagattctgcccgatcctagcaagcccagc |
| | | aagcggagcttcatcgaggacctgctgttcaacaaagtga |
| | | cactggccgacgccggcttcatcaagcagtatggcgattg |
| | | tctgggcgacattgccgccagggatctgatttgcgcccag |
| | | aagtttaacggactgacagtgctgcctcctctgctgaccg |
| | | atgagatgatcgcccagtacacatctgccctgctggccgg |
| | | cacaatcacaagcggctggacatttggagcaggcgccgct |
| | | ctgcagatcccctttgctatgcagatggcctaccggttca |
| | | acggcatcggagtgacccagaatgtgctgtacgagaacca |
| | | gaagctgatcgccaaccagttcaacagcgccatcggcaag |
| | | atccaggacagcctgagcagcacagcaagcgccctgggaa |
| | | agctgcaggacgtggtcaaccacaatgcccaggcactgaa |
| | | caccctggtcaagcagctgtcctccaagttcggcgccatc |
| | | agctctgtgctgaacgatatcctgagcagactggaccctc |
| | | ctgaggccgaggtgcagatcgacagactgatcacaggcag |
| | | actgcagagcctccagacatacgtgacccagcagctgatc |
| | | agagccgccgagattagagcctctgccaatctggccgcca |
| | | ccaagatgtctgagtgtgtgctgggccagagcaagagagt |
| | | ggacttttgcggcaagggctaccacctgatgagcttccct |
| | | cagtctgcccctcacggcgtggtgtttctgcacgtgacat |
| | | atgtgcccgctcaagagaagaatttcaccaccgctccagc |
| | | catctgccacgacggcaaagcccactttcctagagaaggc |
| | | gtgttcgtgtccaacggcacccattggttcgtgacacagc |
| | | ggaacttctacgagccccagatcatcaccaccgacaacac |
| | | cttcgtgtctggcaactgcgacgtcgtgatcggcattgtg |
| | | aacaataccgtgtacgaccctctgcagcccgagctggaca |
| | | gcttcaaagaggaactggacaagtactttaagaaccacac |
| | | aagccccgacgtggacctgggcgatatcagcggaatcaat |
| | | gccagcgtcgtgaacatccagaaagagatcgaccggctga |
| | | acgaggtggccaagaatctgaacgagagcctgatcgacct |
| | | gcaagaactggggaagtacgagcagtacatcaagtggccc |
| | | tggtacatctggctgggctttatcgccggactgattgcca |
| | | tcgtgatggtcacaatcatgctgtgttgcatgaccagctg |
| | | ctgtagctgcctgaagggctgttgtagctgtggcagctgc |
| | | tgcaagttcgacgaggacgattctgagcccgtgctgaagg |
| | | gcgtgaaactgcactacacatgatgactcgagctggtact |
| | | gcatgcacgcaatgctagctgcccctttcccgtcctgggt |
| | | accccgagtctcccccgacctcgggtcccaggtatgctcc |
| | | cacctccacctgccccactcaccacctctgctagttccag |
| | | acacctcccaagcacgcagcaatgcagctcaaaacgctta |
| | | gcctagccacacccccacgggaaacagcagtgattaacct |
| | | ttagcaataaacgaaagtttaactaagctatactaacccc |
| | | agggttggtcaatttcgtgccagccacaccctggagctag |
| | | caaaaaaaaaaaaaaaaaaaaaaaa |

TABLE 16
B. Sequence of one embodiment of an exemplary Omicron XBB.1.16-specific RNA vaccine

| SEQ ID NO. | Brief Description | Sequence |
|---|---|---|
| 134 | Amino acid sequence of RNA-encoded | MFVFLVLLPLVSSQCVNLITRTQSYTNSFTRGVYYPDKVFRSSVLHSTQDLFLPFFSNVTWFHAIHVSGTNGTKR |
| | SARS-COV-2 S protein from an | FDNPALPFNDGVYFASTEKSNIIRGWIFGTTLDSKTQSLLIVNNATNVVIKVCEFQFCNDPFLDVYQKNNKSWM |
| | Omicron XBB.1.16 variant (with PRO | ESEFRVYSSANNCTFEYVSQPFLMDLVGKEGNFKNLREFVFKNIDGYFKIYSKHTPINLERDLPQGFSALEPLVDL |
| | mutations at positions corresponding | PIGINITRFQTLLALHRSYLTPVDSSSGWTAGAAAYYVGYLQPRTFLLKYNENGTITDAVDCALDPLSETKCTLKS |
| | to K986P and V987P of SEQ ID NO: 1; | FTVEKGIYQTSNFRVQPTESIVRFPNITNLCPFHEVFNATTFASVYAWNRKRISNCVADYSVIYNFAPFFAFKCYG |
| | i.e., PRO mutations at positions 982 | VSPTKLNDLCFTNVYADSFVIRGNEVSQIAPGQTGNIADYNYKLPDDFTGCVIAWNSNKLDSKPSGNYNYLYRL |
| | and 983 of SEQ ID NO: 134) | FRKSKLKPFERDISTEIYQAGNRPCNGVAGPNCYSPLQSYGFRPTYGVGHQPYRVVVLSFELLHAPATVCGPKKS |
| | | TNLVKNKCVNFNFNGLTGTGVLTESNKKFLPFQQFGRDIADTTDAVRDPQTLEILDITPCSFGGVSVITPGTNTS |
| | | NQVAVLYQGVNCTEVPVAIHADQLTPTWRVYSTGSNVFQTRAGCLIGAEYVNNSYECDIPIGAGICASYQTQTK |
| | | SHRRARSVASQSIIAYTMSLGAENSVAYSNNSIAIPTNFTISVTTEILPVSMTKTSVDCTMYICGDSTECSNLLLQ |
| | | YGSFCTQLKRALTGIAVEQDKNTQEVFAQVKQIYKTPPIKYFGGFNFSQILPDPSKPSKRSFIEDLLFNKVTLADA |
| | | GFIKQYGDCLGDIAARDLICAQKFNGLTVLPPLLTDEMIAQYTSALLAGTITSGWTFGAGAALQIPFAMQMAYRF |
| | | NGIGVTQNVLYENQKLIANQFNSAIGKIQDSLSSTASALGKLQDVVNHNAQALNTLVKQLSSKFGAISSVLNDIL |
| | | SRLDPPEAEVQIDRLITGRLQSLQTYVTQQLIRAAEIRASANLAATKMSECVLGQSKRVDFCGKGYHLMSFPQSA |
| | | PHGVVFLHVTYVPAQEKNFTTAPAICHDGKAHFPREGVFVSNGTHWFVTQRNFYEPQIITTDNTFVSGNCDVVI |
| | | GIVNNTVYDPLQPELDSFKEELDKYFKNHTSPDVDLGDISGINASVVNIQKEIDRLNEVAKNLNESLIDLQELGKY |
| | | EQYIKWPWYIWLGFIAGLIAIVMVTIMLCCMTSCCSCLKGCCSCGSCCKFDEDDSEPVLKGVKLHYT |
| 135 | RNA sequence encoding a SARS-COV-2 | Auguucguguuccuggugcugcugccucugguguccagccagugugugaaccugaucaccagaacacagucauacaccaacagc |
| | S protein from an Omicron XBB.1.16 | uuuaccagaggcguguacuaccccgacaagguguucagauccagcgugcugcacucuacccaggaccuguuccugccuuucuuca |
| | variant | gcaacgugaccugguuccacgccauccacguguccggcaccaauggcaccaagagauucgacaaccccgcccugcccuucaacgac |
| | | gggguguacuuugccagcaccgagaaguccaacaucaucagaggcuggaucuucggcaccacacuggacagcaagacccagagcc |
| | | ugcugaucgugaacaacgccaccaacguggucaucaaagugugcgaguuccaguucugcaacgaccccuuccuggacgucuacca |
| | | gaagaacaacaagagcuggauggaaagcgaguuccggguguacagcagcgccaacaacugcaccuucgaguacgugucccagccu |
| | | uuccugauggaccugGUGggcaaggagggcaacuucaagaaccugcgcgaguucguguuuaagaacaucgacggcuacuucaa |
| | | gaucuacagcaagcacaccccuaucaaccucgagcgggaucugccucagggcuucucugcucuggaaccccugguggaucugccc |
| | | aucggcaucaacaucacccgguuucagacacugcuggcccugcacagaagcuaccugacaccuguggauagcagcagcggaugga |
| | | cagcuggugccgccgcuuacuaugugggcuaccugcagccuagaaccuuccugcugaaguacaacgagaacggcaccaucaccga |
| | | cgccguggauugugcucuggauccucugagcgagacaaagugcacccugaaguccuucaccguggaaaagggcaucuaccagacc |
| | | agcaacuuccgggugcagcccaccgaauccaucgugcgguuccccaauaucaccaaucugugccccuuccacgagguguucaaugc |
| | | caccaccuucgccucuguguacgccuggaaccggaagcggaucagcaauugcguggccgacuacuccgugaucuacaacuucgcc |
| | | cccuucuucgcauucaagugcuacggcguguccccuaccaagcugaacgaccugugcuucacaaacguguacgccgacagcuucg |
| | | ugauccggggaaacgaagugucacagauugccccuggacagacaggcaacaucgccgacuacaacuacaagcugcccgacgacuu |
| | | caccggcugugugauugccuggaacagcaacaagcuggacuccaaacccagcggcaacuacaauuaccuguaccggcuguuccgg |
| | | aaguccaagcugaagcccuucgagcgggacaucuccaccgagaucuaucaggccggcaacAGGccuuguaacggcguggcaggc |
| | | cccaacugcuacagcccacugcaguccuacggcuuuaggcccacauacggcgugggccaccagcccuacagagugguggugcuga |
| | | gcuucgaacugcugcaugccccugccacagugugcggcccuaagaaaagcaccaaucucgugaagaacaaaugcgugaacuucaa |
| | | cuucaacggccugaccggcaccggcgugcugacagagagcaacaagaaguuccugccauuccagcaguuuggccgggauaucgcc |
| | | gauaccacagacgccguuagagauccccagacacuggaaauccuggacaucaccccuugcagcuucggcggagugucugugauca |
| | | ccccuggcaccaacaccagcaaucagguggcagugcuguaccagggcgugaacuguaccgaagugcccguggccauucacgccga |
| | | ucagcugacaccuacauggcggguguacuccaccggcagcaauguguuucagaccagagccggcugucugaucggagccgaguac |
| | | gugaacaauagcuacgagugcgacauccccaucggcgcuggaaucugcgccagcuaccagacacagacaaagagccaccggagag |
| | | ccagaagcguggccagccagagcaucauugccuacacaaugucucugggcgccgagaacagcguggccuacuccaacaacucuau |
| | | cgcuauccccaccaacuucaccaucagcgugaccacagagauccugccuguguccaugaccaagaccagcguggacugcaccaugu |
| | | acaucugcggcgauuccaccgagugcuccaaccugcugcugcaguacggcagcuucugcacccagcugaaaagagcccugacagg |
| | | gaucgccguggaacaggacaagaacacccaagagguguucgcccaagugaagcagaucuacaagaccccuccuaucaaguacuuc |
| | | ggcggcuucaauuucagccagauucugcccgauccuagcaagcccagcaagcggagcuucaucgaggaccugcuguucaacaaag |
| | | ugacacuggccgacgccggcuucaucaagcaguauggcgauugucugggcgacauugccgccagggaucugauuugcgcccaga |
| | | aguuuaacggacugacagugcugccuccucugcugaccgaugagaugaucgcccaguacacaucugcccugcuggccggcacaau |
| | | cacaagcggcuggacauuuggagcaggcgccgcucugcagauccccuuugcuaugcagauggccuaccgguucaacggcaucgga |
| | | gugacccagaaugugcuguacgagaaccagaagcugaucgccaaccaguucaacagcgccaucggcaagauccaggacagccuga |
| | | gcagcacagcaagcgcccugggaaagcugcaggacguggucaaccacaaugcccaggcacugaacacccuggucaagcagcugucc |
| | | uccaaguucggcgccaucagcucugugcugaacgauauccugagcagacuggacccuccugaggccgaggugcagaucgacagac |
| | | ugaucacaggcagacugcagagccuccagacauacgugacccagcagcugaucagagccgccgagauuagagccucugccaaucu |
| | | ggccgccaccaagaugucugagugugugcugggccagagcaagagaguggacuuuugcggcaagggcuaccaccugaugagcuu |
| | | cccucagucugccccucacggcgugguguuucugcacgugacauaugugcccgcucaagagaagaauuucaccaccgcuccagcc |
| | | aucugccacgacggcaaagcccacuuuccuagagaaggcguguucguguccaacggcacccauugguucgugacacagcggaacu |
| | | ucuacgagccccagaucaucaccaccgacaacaccuucgugucuggcaacugcgacgucgugaucggcauugugaacaauaccgu |
| | | guacgacccucugcagcccgagcuggacagcuucaaagaggaacuggacaaguacuuuaagaaccacacaagccccgacguggac |
| | | cugggcgauaucagcggaaucaaugccagcgucgugaacauccagaaagagaucgaccggcugaacgagguggccaagaaucug |
| | | aacgagagccugaucgaccugcaagaacuggggaaguacgagcaguacaucaaguggcccugguacaucuggcugggcuuuauc |
| | | gccggacugauugccaucgugauggucacaaucaugcuguguugcaugaccagcugcuguagcugccugaagggcuguuguagc |
| | | uguggcagcugcugcaaguucgacgaggacgauucugagcccgugcugaagggcgugaaacugcacuacacaugauga |
| 136 | DNA sequence encoding a SARS-COV-2 | Atgttcgtgttcctggtgctgctgcctctggtgtccagccagtgtgtgaacctgatcaccagaacacagtcatacaccaacagctttaccagagg |
| | S protein from an Omicron XBB.1.16 | cgtgtactaccccgacaaggtgttcagatccagcgtgctgcactctacccaggacctgttcctgcctttcttcagcaacgtgacctggttccacg |
| | variant | ccatccacgtgtccggcaccaatggcaccaagagattcgacaaccccgccctgcccttcaacgacggggtgtactttgccagcaccgagaagt |
| | | ccaacatcatcagaggctggatcttcggcaccacactggacagcaagacccagagcctgctgatcgtgaacaacgccaccaacgtggtcatc |
| | | aaagtgtgcgagttccagttctgcaacgaccccttcctggacgtctaccagaagaacaacaagagctggatggaaagcgagttccgggtgta |
| | | cagcagcgccaacaactgcaccttcgagtacgtgtcccagcctttcctgatggacctgGTGggcaaggagggcaacttcaagaacctgcgc |
| | | gagttcgtgtttaagaacatcgacggctacttcaagatctacagcaagcacacccctatcaacctcgagcgggatctgcctcagggcttctctg |
| | | ctctggaacccctggtggatctgcccatcggcatcaacatcacccggtttcagacactgctggccctgcacagaagctacctgacacctgtgga |
| | | tagcagcagcggatggacagctggtgccgccgcttactatgtgggctacctgcagcctagaaccttcctgctgaagtacaacgagaacggca |
| | | ccatcaccgacgccgtggattgtgctctggatcctctgagcgagacaaagtgcaccctgaagtccttcaccgtggaaaagggcatctaccaga |
| | | ccagcaacttccgggtgcagcccaccgaatccatcgtgcggttccccaatatcaccaatctgtgccccttccacgaggtgttcaatgccaccac |
| | | cttcgcctctgtgtacgcctggaaccggaagcggatcagcaattgcgtggccgactactccgtgatctacaacttcgcccccttcttcgcattca |
| | | agtgctacggcgtgtcccctaccaagctgaacgacctgtgcttcacaaacgtgtacgccgacagcttcgtgatccggggaaacgaagtgtca |
| | | cagattgcccctggacagacaggcaacatcgccgactacaactacaagctgcccgacgacttcaccggctgtgtgattgcctggaacagcaa |
| | | caagctggactccaaacccagcggcaactacaattacctgtaccggctgttccggaagtccaagctgaagcccttcgagcgggacatctcca |
| | | ccgagatctatcaggccggcaacAGGccttgtaacggcgtggcaggccccaactgctacagcccactgcagtcctacggctttaggcccac |
| | | atacggcgtgggccaccagccctacagagtggtggtgctgagcttcgaactgctgcatgcccctgccacagtgtgcggccctaagaaaagca |
| | | ccaatctcgtgaagaacaaatgcgtgaacttcaacttcaacggcctgaccggcaccggcgtgctgacagagagcaacaagaagttcctgcc |
| | | attccagcagtttggccgggatatcgccgataccacagacgccgttagagatccccagacactggaaatcctggacatcaccccttgcagctt |
| | | cggcggagtgtctgtgatcacccctggcaccaacaccagcaatcaggtggcagtgctgtaccagggcgtgaactgtaccgaagtgcccgtg |
| | | gccattcacgccgatcagctgacacctacatggcgggtgtactccaccggcagcaatgtgtttcagaccagagccggctgtctgatcggagcc |
| | | gagtacgtgaacaatagctacgagtgcgacatccccatcggcgctggaatctgcgccagctaccagacacagacaaagagccaccggaga |
| | | gccagaagcgtggccagccagagcatcattgcctacacaatgtctctgggcgccgagaacagcgtggcctactccaacaactctatcgctatc |
| | | cccaccaacttcaccatcagcgtgaccacagagatcctgcctgtgtccatgaccaagaccagcgtggactgcaccatgtacatctgcggcgat |
| | | tccaccgagtgctccaacctgctgctgcagtacggcagcttctgcacccagctgaaaagagccctgacagggatcgccgtggaacaggaca |
| | | agaacacccaagaggtgttcgcccaagtgaagcagatctacaagacccctcctatcaagtacttcggcggcttcaatttcagccagattctgcc |
| | | cgatcctagcaagcccagcaagcggagcttcatcgaggacctgctgttcaacaaagtgacactggccgacgccggcttcatcaagcagtatg |
| | | gcgattgtctgggcgacattgccgccagggatctgatttgcgcccagaagtttaacggactgacagtgctgcctcctctgctgaccgatgagat |
| | | gatcgcccagtacacatctgccctgctggccggcacaatcacaagcggctggacatttggagcaggcgccgctctgcagatcccctttgctat |
| | | gcagatggcctaccggttcaacggcatcggagtgacccagaatgtgctgtacgagaaccagaagctgatcgccaaccagttcaacagcgcc |
| | | atcggcaagatccaggacagcctgagcagcacagcaagcgccctgggaaagctgcaggacgtggtcaaccacaatgcccaggcactgaa |
| | | caccctggtcaagcagctgtcctccaagttcggcgccatcagctctgtgctgaacgatatcctgagcagactggaccctcctgaggccgaggt |
| | | gcagatcgacagactgatcacaggcagactgcagagcctccagacatacgtgacccagcagctgatcagagccgccgagattagagcctct |
| | | gccaatctggccgccaccaagatgtctgagtgtgtgctgggccagagcaagagagtggacttttgcggcaagggctaccacctgatgagctt |
| | | ccctcagtctgcccctcacggcgtggtgtttctgcacgtgacatatgtgcccgctcaagagaagaatttcaccaccgctccagccatctgccacg |
| | | acggcaaagcccactttcctagagaaggcgtgttcgtgtccaacggcacccattggttcgtgacacagcggaacttctacgagccccagatca |
| | | tcaccaccgacaacaccttcgtgtctggcaactgcgacgtcgtgatcggcattgtgaacaataccgtgtacgaccctctgcagcccgagctgg |
| | | acagcttcaaagaggaactggacaagtactttaagaaccacacaagccccgacgtggacctgggcgatatcagcggaatcaatgccagcgt |
| | | cgtgaacatccagaaagagatcgaccggctgaacgaggtggccaagaatctgaacgagagcctgatcgacctgcaagaactggggaagt |
| | | acgagcagtacatcaagtggccctggtacatctggctgggctttatcgccggactgattgccatcgtgatggtcacaatcatgctgtgttgcatg |
| | | accagctgctgtagctgcctgaagggctgttgtagctgtggcagctgctgcaagttcgacgaggacgattctgagcccgtgctgaagggcgt |
| | | gaaactgcactacacatgatga |
| 137 | Full length RNA construct encoding a | Agaauaaacuaguauucuucugguccccacagacucagagagaacccgccaccauguucguguuccuggugcugcugccucugg |
| | SARS-COV-2 S protein from an | uguccagccagugugugaaccugaucaccagaacacagucauacaccaacagcuuuaccagaggcguguacuaccccgacaaggu |
| | Omicron XBB.1.16 variant | guucagauccagcgugcugcacucuacccaggaccuguuccugccuuucuucagcaacgugaccugguuccacgccauccacgug |
| | | uccggcaccaauggcaccaagagauucgacaaccccgcccugcccuucaacgacgggguguacuuugccagcaccgagaaguccaa |
| | | caucaucagaggcuggaucuucggcaccacacuggacagcaagacccagagccugcugaucgugaacaacgccaccaacgugguc |
| | | aucaaagugugcgaguuccaguucugcaacgaccccuuccuggacgucuaccagaagaacaacaagagcuggauggaaagcgag |
| | | uuccggguguacagcagcgccaacaacugcaccuucgaguacgugucccagccuuuccugauggaccugGUGggcaaggagggc |
| | | aacuucaagaaccugcgcgaguucguguuuaagaacaucgacggcuacuucaagaucuacagcaagcacaccccuaucaaccucg |
| | | agcgggaucugccucagggcuucucugcucuggaaccccugguggaucugcccaucggcaucaacaucacccgguuucagacacu |
| | | gcuggcccugcacagaagcuaccugacaccuguggauagcagcagcggauggacagcuggugccgccgcuuacuaugugggcua |
| | | ccugcagccuagaaccuuccugcugaaguacaacgagaacggcaccaucaccgacgccguggauugugcucuggauccucugagc |
| | | gagacaaagugcacccugaaguccuucaccguggaaaagggcaucuaccagaccagcaacuuccgggugcagcccaccgaaucca |
| | | ucgugcgguuccccaauaucaccaaucugugccccuuccacgagguguucaaugccaccaccuucgccucuguguacgccuggaa |
| | | ccggaagcggaucagcaauugcguggccgacuacuccgugaucuacaacuucgcccccuucuucgcauucaagugcuacggcgug |
| | | uccccuaccaagcugaacgaccugugcuucacaaacguguacgccgacagcuucgugauccggggaaacgaagugucacagauug |
| | | ccccuggacagacaggcaacaucgccgacuacaacuacaagcugcccgacgacuucaccggcugugugauugccuggaacagcaac |
| | | aagcuggacuccaaacccagcggcaacuacaauuaccuguaccggcuguuccggaaguccaagcugaagcccuucgagcgggaca |
| | | ucuccaccgagaucuaucaggccggcaacAGGccuuguaacggcguggcaggccccaacugcuacagcccacugcaguccuacgg |
| | | cuuuaggcccacauacggcgugggccaccagcccuacagagugguggugcugagcuucgaacugcugcaugccccugccacagug |
| | | ugcggcccuaagaaaagcaccaaucucgugaagaacaaaugcgugaacuucaacuucaacggccugaccggcaccggcgugcuga |
| | | cagagagcaacaagaaguuccugccauuccagcaguuuggccgggauaucgccgauaccacagacgccguuagagauccccagac |
| | | acuggaaauccuggacaucaccccuugcagcuucggcggagugucugugaucaccccuggcaccaacaccagcaaucagguggca |
| | | gugcuguaccagggcgugaacuguaccgaagugcccguggccauucacgccgaucagcugacaccuacauggcggguguacucca |
| | | ccggcagcaauguguuucagaccagagccggcugucugaucggagccgaguacgugaacaauagcuacgagugcgacauccccau |
| | | cggcgcuggaaucugcgccagcuaccagacacagacaaagagccaccggagagccagaagcguggccagccagagcaucauugcc |
| | | uacacaaugucucugggcgccgagaacagcguggccuacuccaacaacucuaucgcuauccccaccaacuucaccaucagcgugac |
| | | cacagagauccugccuguguccaugaccaagaccagcguggacugcaccauguacaucugcggcgauuccaccgagugcuccaac |
| | | cugcugcugcaguacggcagcuucugcacccagcugaaaagagcccugacagggaucgccguggaacaggacaagaacacccaag |
| | | agguguucgcccaagugaagcagaucuacaagaccccuccuaucaaguacuucggcggcuucaauuucagccagauucugcccga |
| | | uccuagcaagcccagcaagcggagcuucaucgaggaccugcuguucaacaaagugacacuggccgacgccggcuucaucaagcag |
| | | uauggcgauugucugggcgacauugccgccagggaucugauuugcgcccagaaguuuaacggacugacagugcugccuccucug |
| | | cugaccgaugagaugaucgcccaguacacaucugcccugcuggccggcacaaucacaagcggcuggacauuuggagcaggcgccg |
| | | cucugcagauccccuuugcuaugcagauggccuaccgguucaacggcaucggagugacccagaaugugcuguacgagaaccagaa |
| | | gcugaucgccaaccaguucaacagcgccaucggcaagauccaggacagccugagcagcacagcaagcgcccugggaaagcugcag |
| | | gacguggucaaccacaaugcccaggcacugaacacccuggucaagcagcuguccuccaaguucggcgccaucagcucugugcuga |
| | | acgauauccugagcagacuggacccuccugaggccgaggugcagaucgacagacugaucacaggcagacugcagagccuccagac |
| | | auacgugacccagcagcugaucagagccgccgagauuagagccucugccaaucuggccgccaccaagaugucugagugugugcug |
| | | ggccagagcaagagaguggacuuuugcggcaagggcuaccaccugaugagcuucccucagucugccccucacggcgugguguuu |
| | | cugcacgugacauaugugcccgcucaagagaagaauuucaccaccgcuccagccaucugccacgacggcaaagcccacuuuccuag |
| | | agaaggcguguucguguccaacggcacccauugguucgugacacagcggaacuucuacgagccccagaucaucaccaccgacaac |
| | | accuucgugucuggcaacugcgacgucgugaucggcauugugaacaauaccguguacgacccucugcagcccgagcuggacagcu |
| | | ucaaagaggaacuggacaaguacuuuaagaaccacacaagccccgacguggaccugggcgauaucagcggaaucaaugccagcgu |
| | | cgugaacauccagaaagagaucgaccggcugaacgagguggccaagaaucugaacgagagccugaucgaccugcaagaacuggg |
| | | gaaguacgagcaguacaucaaguggcccugguacaucuggcugggcuuuaucgccggacugauugccaucgugauggucacaau |
| | | caugcuguguugcaugaccagcugcuguagcugccugaagggcuguuguagcuguggcagcugcugcaaguucgacgaggacg |
| | | auucugagcccgugcugaagggcgugaaacugcacuacacaugaugacucgagcugguacugcaugcacgcaaugcuagcugcc |
| | | ccuuucccguccuggguaccccgagucucccccgaccucgggucccagguaugcucccaccuccaccugccccacucaccaccucug |
| | | cuaguuccagacaccucccaagcacgcagcaaugcagcucaaaacgcuuagccuagccacacccccacgggaaacagcagugauua |
| | | accuuuagcaauaaacgaaaguuuaacuaagcuauacuaaccccaggguuggucaauuucgugccagccacacccuggagcuagc |
| | | aaaaaaaaaaaaaaaaaaaaaaaaaaaaaagcauaugacuaaaaaaaaaaaaaaaaaaaaaaaaaaaaaaaaaaaaaaaaaaaa |
| | | aaaaaaaaaaaaaaaaaaaaaaaaa |
| 138 | Full length DNA construct encoding a | Agaataaactagtattcttctggtccccacagactcagagagaacccgccaccatgttcgtgttcctggtgctgctgcctctggtgtccagccag |
| | SARS-COV-2 S protein from an | tgtgtgaacctgatcaccagaacacagtcatacaccaacagctttaccagaggcgtgtactaccccgacaaggtgttcagatccagcgtgctg |
| | Omicron XBB.1.16 variant | cactctacccaggacctgttcctgcctttcttcagcaacgtgacctggttccacgccatccacgtgtccggcaccaatggcaccaagagattcga |
| | | caaccccgccctgcccttcaacgacggggtgtactttgccagcaccgagaagtccaacatcatcagaggctggatcttcggcaccacactgga |
| | | cagcaagacccagagcctgctgatcgtgaacaacgccaccaacgtggtcatcaaagtgtgcgagttccagttctgcaacgaccccttcctgga |
| | | cgtctaccagaagaacaacaagagctggatggaaagcgagttccgggtgtacagcagcgccaacaactgcaccttcgagtacgtgtcccag |
| | | cctttcctgatggacctgGTGggcaaggagggcaacttcaagaacctgcgcgagttcgtgtttaagaacatcgacggctacttcaagatcta |
| | | cagcaagcacacccctatcaacctcgagcgggatctgcctcagggcttctctgctctggaacccctggtggatctgcccatcggcatcaacatc |
| | | acccggtttcagacactgctggccctgcacagaagctacctgacacctgtggatagcagcagcggatggacagctggtgccgccgcttactat |
| | | gtgggctacctgcagcctagaaccttcctgctgaagtacaacgagaacggcaccatcaccgacgccgtggattgtgctctggatcctctgagc |
| | | gagacaaagtgcaccctgaagtccttcaccgtggaaaagggcatctaccagaccagcaacttccgggtgcagcccaccgaatccatcgtgc |
| | | ggttccccaatatcaccaatctgtgccccttccacgaggtgttcaatgccaccaccttcgcctctgtgtacgcctggaaccggaagcggatcagc |
| | | aattgcgtggccgactactccgtgatctacaacttcgcccccttcttcgcattcaagtgctacggcgtgtcccctaccaagctgaacgacctgtg |
| | | cttcacaaacgtgtacgccgacagcttcgtgatccggggaaacgaagtgtcacagattgcccctggacagacaggcaacatcgccgactaca |
| | | actacaagctgcccgacgacttcaccggctgtgtgattgcctggaacagcaacaagctggactccaaacccagcggcaactacaattacctgt |
| | | accggctgttccggaagtccaagctgaagcccttcgagcgggacatctccaccgagatctatcaggccggcaacAGGccttgtaacggcgt |
| | | ggcaggccccaactgctacagcccactgcagtcctacggctttaggcccacatacggcgtgggccaccagccctacagagtggtggtgctga |
| | | gcttcgaactgctgcatgcccctgccacagtgtgcggccctaagaaaagcaccaatctcgtgaagaacaaatgcgtgaacttcaacttcaacg |
| | | gcctgaccggcaccggcgtgctgacagagagcaacaagaagttcctgccattccagcagtttggccgggatatcgccgataccacagacgc |
| | | cgttagagatccccagacactggaaatcctggacatcaccccttgcagcttcggcggagtgtctgtgatcacccctggcaccaacaccagcaa |
| | | tcaggtggcagtgctgtaccagggcgtgaactgtaccgaagtgcccgtggccattcacgccgatcagctgacacctacatggcgggtgtactc |
| | | caccggcagcaatgtgtttcagaccagagccggctgtctgatcggagccgagtacgtgaacaatagctacgagtgcgacatccccatcggcg |
| | | ctggaatctgcgccagctaccagacacagacaaagagccaccggagagccagaagcgtggccagccagagcatcattgcctacacaatgt |
| | | ctctgggcgccgagaacagcgtggcctactccaacaactctatcgctatccccaccaacttcaccatcagcgtgaccacagagatcctgcctgt |
| | | gtccatgaccaagaccagcgtggactgcaccatgtacatctgcggcgattccaccgagtgctccaacctgctgctgcagtacggcagcttctg |
| | | cacccagctgaaaagagccctgacagggatcgccgtggaacaggacaagaacacccaagaggtgttcgcccaagtgaagcagatctaca |
| | | agacccctcctatcaagtacttcggcggcttcaatttcagccagattctgcccgatcctagcaagcccagcaagcggagcttcatcgaggacct |
| | | gctgttcaacaaagtgacactggccgacgccggcttcatcaagcagtatggcgattgtctgggcgacattgccgccagggatctgatttgcgc |
| | | ccagaagtttaacggactgacagtgctgcctcctctgctgaccgatgagatgatcgcccagtacacatctgccctgctggccggcacaatcac |
| | | aagcggctggacatttggagcaggcgccgctctgcagatcccctttgctatgcagatggcctaccggttcaacggcatcggagtgacccaga |
| | | atgtgctgtacgagaaccagaagctgatcgccaaccagttcaacagcgccatcggcaagatccaggacagcctgagcagcacagcaagcg |
| | | ccctgggaaagctgcaggacgtggtcaaccacaatgcccaggcactgaacaccctggtcaagcagctgtcctccaagttcggcgccatcagc |
| | | tctgtgctgaacgatatcctgagcagactggaccctcctgaggccgaggtgcagatcgacagactgatcacaggcagactgcagagcctcca |
| | | gacatacgtgacccagcagctgatcagagccgccgagattagagcctctgccaatctggccgccaccaagatgtctgagtgtgtgctgggcc |
| | | agagcaagagagtggacttttgcggcaagggctaccacctgatgagcttccctcagtctgcccctcacggcgtggtgtttctgcacgtgacata |
| | | tgtgcccgctcaagagaagaatttcaccaccgctccagccatctgccacgacggcaaagcccactttcctagagaaggcgtgttcgtgtccaa |
| | | cggcacccattggttcgtgacacagcggaacttctacgagccccagatcatcaccaccgacaacaccttcgtgtctggcaactgcgacgtcgt |
| | | gatcggcattgtgaacaataccgtgtacgaccctctgcagcccgagctggacagcttcaaagaggaactggacaagtactttaagaaccaca |
| | | caagccccgacgtggacctgggcgatatcagcggaatcaatgccagcgtcgtgaacatccagaaagagatcgaccggctgaacgaggtgg |
| | | ccaagaatctgaacgagagcctgatcgacctgcaagaactggggaagtacgagcagtacatcaagtggccctggtacatctggctgggcttt |
| | | atcgccggactgattgccatcgtgatggtcacaatcatgctgtgttgcatgaccagctgctgtagctgcctgaagggctgttgtagctgtggca |
| | | gctgctgcaagttcgacgaggacgattctgagcccgtgctgaagggcgtgaaactgcactacacatgatgactcgagctggtactgcatgca |
| | | cgcaatgctagctgcccctttcccgtcctgggtaccccgagtctcccccgacctcgggtcccaggtatgctcccacctccacctgccccactcac |
| | | cacctctgctagttccagacacctcccaagcacgcagcaatgcagctcaaaacgcttagcctagccacacccccacgggaaacagcagtgatt |
| | | aacctttagcaataaacgaaagtttaactaagctatactaaccccagggttggtcaatttcgtgccagccacaccctggagctagcaaaaaaa |
| | | aaaaaaaaaaaaaaaaaaaaaaagcatatgactaaaaaaaaaaaaaaaaaaaaaaaaaaaaaaaaaaaaaaaaaaaaaaaaaaaa |
| | | aaaaaaaaaaaaaaaaa |

C. Sequence of one embodiment of an exemplary Omicron XBB.2.3-specific RNA vaccine

| SEQ ID NO. | Brief Description | Sequence |
|---|---|---|
| 139 | Amino acid sequence of RNA-encoded | MFVFLVLLPLVSSQCVNLITRTQSYTNSFTRGVYYPDKVFRSSVLHSTQDLFLPFFSNVTWFHAIHVSGTNGTKR |
| | SARS-COV-2 S protein from an | FDNPALPFNDGVYFASTEKSNIIRGWIFGTTLDSKTQSLLIVNNATNVVIKVCEFQFCNDPFLDVYQKNNKSWM |
| | Omicron XBB.2.3 variant (with PRO | ESEFRVYSSANNCTFEYVSQPFLMDLEGKEGNFKNLREFVFKNIDGYFKIYSKHTPINLERDLPQGFSALEPLVDL |
| | mutations at positions corresponding | PIGINITRFQTLLALHRSYLTPGGSSSGWTAGAAAYYVGYLQPRTFLLKYNENGTITDAVDCALDPLSETKCTLKS |
| | to K986P and V987P of SEQ ID NO: 1; | FTVEKGIYQTSNFRVQPTESIVRFPNITNLCPFHEVFNATTFASVYAWNRKRISNCVADYSVIYNFAPFFAFKCYG |
| | i.e., PRO mutations at positions 982 | VSPTKLNDLCFTNVYADSFVIRGNEVSQIAPGQTGNIADYNYKLPDDFTGCVIAWNSNKLDSKPSGNYNYLYRL |
| | and 983 of SEQ ID NO: 139) | FRKSKLKPFERDISTEIYQAGNKPCNGVAGPNCYSPLQSYGFRPTYGVGHQPYRVVVLSFELLHASATVCGPKKS |
| | | TNLVKNKCVNFNFNGLTGTGVLTESNKKFLPFQQFGRDIADTTDAVRDPQTLEILDITPCSFGGVSVITPGTNTS |
| | | NQVAVLYQGVNCTEVPVAIHADQLTPTWRVYSTGSNVFQTRAGCLIGAEYVNNSYECDIPIGAGICASYQTQTK |
| | | SHRRARSVASQSIIAYTMSLGAENSVAYSNNSIAIPTNFTISVTTEILPVSMTKTSVDCTMYICGDSTECSNLLLQ |
| | | YGSFCTQLKRALTGIAVEQDKNTQEVFAQVKQIYKTPPIKYFGGFNFSQILPDPSKPSKRSFIEDLLFNKVTLADA |
| | | GFIKQYGDCLGDIAARDLICAQKFNGLTVLPPLLTDEMIAQYTSALLAGTITSGWTFGAGAALQIPFAMQMAYRF |
| | | NGIGVTQNVLYENQKLIANQFNSAIGKIQDSLSSTASALGKLQDVVNHNAQALNTLVKQLSSKFGAISSVLNDIL |
| | | SRLDPPEAEVQIDRLITGRLQSLQTYVTQQLIRAAEIRASANLAATKMSECVLGQSKRVDFCGKGYHLMSFPQSA |
| | | PHGVVFLHVTYVPAQEKNFTTAPAICHDGKAHFPREGVFVSNGTHWFVTQRNFYEPQIITTDNTFVSGNCDVVI |
| | | GIVNNTVYDPLQPELDSFKEELDKYFKNHTSPDVDLGDISGINASVVNIQKEIDRLNEVAKNLNESLIDLQELGKY |
| | | EQYIKWPWYIWLGFIAGLIAIVMVTIMLCCMTSCCSCLKGCCSCGSCCKFDEDDSEPVLKGVKLHYT |
| 140 | RNA sequence encoding a SARS-COV-2 | auguucguguuccuggugcugcugccucugguguccagccagugugugaaccugaucaccagaacacagucauacaccaacagc |
| | S protein from an Omicron XBB.2.3 | uuuaccagaggcguguacuaccccgacaagguguucagauccagcgugcugcacucuacccaggaccuguuccugccuuucuuca |
| | variant | gcaacgugaccugguuccacgccauccacguguccggcaccaauggcaccaagagauucgacaaccccgcccugcccuucaacgac |
| | | gggguguacuuugccagcaccgagaaguccaacaucaucagaggcuggaucuucggcaccacacuggacagcaagacccagagcc |
| | | ugcugaucgugaacaacgccaccaacguggucaucaaagugugcgaguuccaguucugcaacgaccccuuccuggacgucuacca |
| | | gaagaacaacaagagcuggauggaaagcgaguuccggguguacagcagcgccaacaacugcaccuucgaguacgugucccagccu |
| | | uuccugauggaccuggaaggcaaggagggcaacuucaagaaccugcgcgaguucguguuuaagaacaucgacggcuacuucaag |
| | | aucuacagcaagcacaccccuaucaaccucgagcgggaucugccucagggcuucucugcucuggaaccccugguggaucugccca |
| | | ucggcaucaacaucacccgguuucagacacugcuggcccugcacagaagcuaccugacaccuggcggcagcagcagcggauggac |
| | | agcuggugccgccgcuuacuaugugggcuaccugcagccuagaaccuuccugcugaaguacaacgagaacggcaccaucaccgac |
| | | gccguggauugugcucuggauccucugagcgagacaaagugcacccugaaguccuucaccguggaaaagggcaucuaccagacca |
| | | gcaacuuccgggugcagcccaccgaauccaucgugcgguuccccaauaucaccaaucugugccccuuccacgagguguucaaugcc |
| | | accaccuucgccucuguguacgccuggaaccggaagcggaucagcaauugcguggccgacuacuccgugaucuacaacuucgccc |
| | | ccuucuucgcauucaagugcuacggcguguccccuaccaagcugaacgaccugugcuucacaaacguguacgccgacagcuucgu |
| | | gauccggggaaacgaagugucacagauugccccuggacagacaggcaacaucgccgacuacaacuacaagcugcccgacgacuuc |
| | | accggcugugugauugccuggaacagcaacaagcuggacuccaaacccagcggcaacuacaauuaccuguaccggcuguuccgga |
| | | aguccaagcugaagcccuucgagcgggacaucuccaccgagaucuaucaggccggcaacaagccuuguaacggcguggcaggccc |
| | | caacugcuacagcccacugcaguccuacggcuuuaggcccacauacggcgugggccaccagcccuacagagugguggugcugagc |
| | | uucgaacugcugcaugccucagccacagugugcggcccuaagaaaagcaccaaucucgugaagaacaaaugcgugaacuucaacu |
| | | ucaacggccugaccggcaccggcgugcugacagagagcaacaagaaguuccugccauuccagcaguuuggccgggauaucgccga |
| | | uaccacagacgccguuagagauccccagacacuggaaauccuggacaucaccccuugcagcuucggcggagugucugugaucacc |
| | | ccuggcaccaacaccagcaaucagguggcagugcuguaccagggcgugaacuguaccgaagugcccguggccauucacgccgauc |
| | | agcugacaccuacauggcggguguacuccaccggcagcaauguguuucagaccagagccggcugucugaucggagccgaguacg |
| | | ugaacaauagcuacgagugcgacauccccaucggcgcuggaaucugcgccagcuaccagacacagacaaagagccaccggagagc |
| | | cagaagcguggccagccagagcaucauugccuacacaaugucucugggcgccgagaacagcguggccuacuccaacaacucuauc |
| | | gcuauccccaccaacuucaccaucagcgugaccacagagauccugccuguguccaugaccaagaccagcguggacugcaccaugua |
| | | caucugcggcgauuccaccgagugcuccaaccugcugcugcaguacggcagcuucugcacccagcugaaaagagcccugacaggg |
| | | aucgccguggaacaggacaagaacacccaagagguguucgcccaagugaagcagaucuacaagaccccuccuaucaaguacuucg |
| | | gcggcuucaauuucagccagauucugcccgauccuagcaagcccagcaagcggagcuucaucgaggaccugcuguucaacaaagu |
| | | gacacuggccgacgccggcuucaucaagcaguauggcgauugucugggcgacauugccgccagggaucugauuugcgcccagaa |
| | | guuuaacggacugacagugcugccuccucugcugaccgaugagaugaucgcccaguacacaucugcccugcuggccggcacaauc |
| | | acaagcggcuggacauuuggagcaggcgccgcucugcagauccccuuugcuaugcagauggccuaccgguucaacggcaucgga |
| | | gugacccagaaugugcuguacgagaaccagaagcugaucgccaaccaguucaacagcgccaucggcaagauccaggacagccuga |
| | | gcagcacagcaagcgcccugggaaagcugcaggacguggucaaccacaaugcccaggcacugaacacccuggucaagcagcugucc |
| | | uccaaguucggcgccaucagcucugugcugaacgauauccugagcagacuggacccuccugaggccgaggugcagaucgacagac |
| | | ugaucacaggcagacugcagagccuccagacauacgugacccagcagcugaucagagccgccgagauuagagccucugccaaucu |
| | | ggccgccaccaagaugucugagugugugcugggccagagcaagagaguggacuuuugcggcaagggcuaccaccugaugagcuu |
| | | cccucagucugccccucacggcgugguguuucugcacgugacauaugugcccgcucaagagaagaauuucaccaccgcuccagcc |
| | | aucugccacgacggcaaagcccacuuuccuagagaaggcguguucguguccaacggcacccauugguucgugacacagcggaacu |
| | | ucuacgagccccagaucaucaccaccgacaacaccuucgugucuggcaacugcgacgucgugaucggcauugugaacaauaccgu |
| | | guacgacccucugcagcccgagcuggacagcuucaaagaggaacuggacaaguacuuuaagaaccacacaagccccgacguggac |
| | | cugggcgauaucagcggaaucaaugccagcgucgugaacauccagaaagagaucgaccggcugaacgagguggccaagaaucug |
| | | aacgagagccugaucgaccugcaagaacuggggaaguacgagcaguacaucaaguggcccugguacaucuggcugggcuuuauc |
| | | gccggacugauugccaucgugauggucacaaucaugcuguguugcaugaccagcugcuguagcugccugaagggcuguuguagc |
| | | uguggcagcugcugcaaguucgacgaggacgauucugagcccgugcugaagggcgugaaacugcacuacacaugauga |
| 141 | DNA sequence encoding a SARS-COV-2 | atgttcgtgttcctggtgctgctgcctctggtgtccagccagtgtgtgaacctgatcaccagaacacagtcatacaccaacagctttaccagagg |
| | S protein from an Omicron XBB.2.3 | cgtgtactaccccgacaaggtgttcagatccagcgtgctgcactctacccaggacctgttcctgcctttcttcagcaacgtgacctggttccacg |
| | variant | ccatccacgtgtccggcaccaatggcaccaagagattcgacaaccccgccctgcccttcaacgacggggtgtactttgccagcaccgagaagt |
| | | ccaacatcatcagaggctggatcttcggcaccacactggacagcaagacccagagcctgctgatcgtgaacaacgccaccaacgtggtcatc |
| | | aaagtgtgcgagttccagttctgcaacgaccccttcctggacgtctaccagaagaacaacaagagctggatggaaagcgagttccgggtgta |
| | | cagcagcgccaacaactgcaccttcgagtacgtgtcccagcctttcctgatggacctggaaggcaaggagggcaacttcaagaacctgcgcg |
| | | agttcgtgtttaagaacatcgacggctacttcaagatctacagcaagcacacccctatcaacctcgagcgggatctgcctcagggcttctctgct |
| | | ctggaacccctggtggatctgcccatcggcatcaacatcacccggtttcagacactgctggccctgcacagaagctacctgacacctggcggc |
| | | agcagcagcggatggacagctggtgccgccgcttactatgtgggctacctgcagcctagaaccttcctgctgaagtacaacgagaacggcac |
| | | catcaccgacgccgtggattgtgctctggatcctctgagcgagacaaagtgcaccctgaagtccttcaccgtggaaaagggcatctaccagac |
| | | cagcaacttccgggtgcagcccaccgaatccatcgtgcggttccccaatatcaccaatctgtgccccttccacgaggtgttcaatgccaccacct |
| | | tcgcctctgtgtacgcctggaaccggaagcggatcagcaattgcgtggccgactactccgtgatctacaacttcgcccccttcttcgcattcaag |
| | | tgctacggcgtgtcccctaccaagctgaacgacctgtgcttcacaaacgtgtacgccgacagcttcgtgatccggggaaacgaagtgtcaca |
| | | gattgcccctggacagacaggcaacatcgccgactacaactacaagctgcccgacgacttcaccggctgtgtgattgcctggaacagcaaca |
| | | agctggactccaaacccagcggcaactacaattacctgtaccggctgttccggaagtccaagctgaagcccttcgagcgggacatctccacc |
| | | gagatctatcaggccggcaacaagccttgtaacggcgtggcaggccccaactgctacagcccactgcagtcctacggctttaggcccacata |
| | | cggcgtgggccaccagccctacagagtggtggtgctgagcttcgaactgctgcatgcctcagccacagtgtgcggccctaagaaaagcacc |
| | | aatctcgtgaagaacaaatgcgtgaacttcaacttcaacggcctgaccggcaccggcgtgctgacagagagcaacaagaagttcctgccatt |
| | | ccagcagtttggccgggatatcgccgataccacagacgccgttagagatccccagacactggaaatcctggacatcaccccttgcagcttcgg |
| | | cggagtgtctgtgatcacccctggcaccaacaccagcaatcaggtggcagtgctgtaccagggcgtgaactgtaccgaagtgcccgtggcca |
| | | ttcacgccgatcagctgacacctacatggcgggtgtactccaccggcagcaatgtgtttcagaccagagccggctgtctgatcggagccgagt |
| | | acgtgaacaatagctacgagtgcgacatccccatcggcgctggaatctgcgccagctaccagacacagacaaagagccaccggagagcca |
| | | gaagcgtggccagccagagcatcattgcctacacaatgtctctgggcgccgagaacagcgtggcctactccaacaactctatcgctatcccca |
| | | ccaacttcaccatcagcgtgaccacagagatcctgcctgtgtccatgaccaagaccagcgtggactgcaccatgtacatctgcggcgattcca |
| | | ccgagtgctccaacctgctgctgcagtacggcagcttctgcacccagctgaaaagagccctgacagggatcgccgtggaacaggacaagaa |
| | | cacccaagaggtgttcgcccaagtgaagcagatctacaagacccctcctatcaagtacttcggcggcttcaatttcagccagattctgcccgat |
| | | cctagcaagcccagcaagcggagcttcatcgaggacctgctgttcaacaaagtgacactggccgacgccggcttcatcaagcagtatggcg |
| | | attgtctgggcgacattgccgccagggatctgatttgcgcccagaagtttaacggactgacagtgctgcctcctctgctgaccgatgagatgat |
| | | cgcccagtacacatctgccctgctggccggcacaatcacaagcggctggacatttggagcaggcgccgctctgcagatcccctttgctatgca |
| | | gatggcctaccggttcaacggcatcggagtgacccagaatgtgctgtacgagaaccagaagctgatcgccaaccagttcaacagcgccatc |
| | | ggcaagatccaggacagcctgagcagcacagcaagcgccctgggaaagctgcaggacgtggtcaaccacaatgcccaggcactgaacac |
| | | cctggtcaagcagctgtcctccaagttcggcgccatcagctctgtgctgaacgatatcctgagcagactggaccctcctgaggccgaggtgca |
| | | gatcgacagactgatcacaggcagactgcagagcctccagacatacgtgacccagcagctgatcagagccgccgagattagagcctctgcc |
| | | aatctggccgccaccaagatgtctgagtgtgtgctgggccagagcaagagagtggacttttgcggcaagggctaccacctgatgagcttccct |
| | | cagtctgcccctcacggcgtggtgtttctgcacgtgacatatgtgcccgctcaagagaagaatttcaccaccgctccagccatctgccacgacg |
| | | gcaaagcccactttcctagagaaggcgtgttcgtgtccaacggcacccattggttcgtgacacagcggaacttctacgagccccagatcatca |
| | | ccaccgacaacaccttcgtgtctggcaactgcgacgtcgtgatcggcattgtgaacaataccgtgtacgaccctctgcagcccgagctggaca |
| | | gcttcaaagaggaactggacaagtactttaagaaccacacaagccccgacgtggacctgggcgatatcagcggaatcaatgccagcgtcgt |
| | | gaacatccagaaagagatcgaccggctgaacgaggtggccaagaatctgaacgagagcctgatcgacctgcaagaactggggaagtacg |
| | | agcagtacatcaagtggccctggtacatctggctgggctttatcgccggactgattgccatcgtgatggtcacaatcatgctgtgttgcatgacc |
| | | agctgctgtagctgcctgaagggctgttgtagctgtggcagctgctgcaagttcgacgaggacgattctgagcccgtgctgaagggcgtgaa |
| | | actgcactacacatgatga |
| 142 | Full length RNA construct encoding a | Agaauaaacuaguauucuucugguccccacagacucagagagaacccgccaccauguucguguuccuggugcugcugccucugg |
| | SARS-COV-2 S protein from an | uguccagccagugugugaaccugaucaccagaacacagucauacaccaacagcuuuaccagaggcguguacuaccccgacaaggu |
| | Omicron XBB.2.3 variant | guucagauccagcgugcugcacucuacccaggaccuguuccugccuuucuucagcaacgugaccugguuccacgccauccacgug |
| | | uccggcaccaauggcaccaagagauucgacaaccccgcccugcccuucaacgacgggguguacuuugccagcaccgagaaguccaa |
| | | caucaucagaggcuggaucuucggcaccacacuggacagcaagacccagagccugcugaucgugaacaacgccaccaacgugguc |
| | | aucaaagugugcgaguuccaguucugcaacgaccccuuccuggacgucuaccagaagaacaacaagagcuggauggaaagcgag |
| | | uuccggguguacagcagcgccaacaacugcaccuucgaguacgugucccagccuuuccugauggaccuggaaggcaaggagggca |
| | | acuucaagaaccugcgcgaguucguguuuaagaacaucgacggcuacuucaagaucuacagcaagcacaccccuaucaaccucga |
| | | gcgggaucugccucagggcuucucugcucuggaaccccugguggaucugcccaucggcaucaacaucacccgguuucagacacug |
| | | cuggcccugcacagaagcuaccugacaccuggcggcagcagcagcggauggacagcuggugccgccgcuuacuaugugggcuacc |
| | | ugcagccuagaaccuuccugcugaaguacaacgagaacggcaccaucaccgacgccguggauugugcucuggauccucugagcga |
| | | gacaaagugcacccugaaguccuucaccguggaaaagggcaucuaccagaccagcaacuuccgggugcagcccaccgaauccaucg |
| | | ugcgguuccccaauaucaccaaucugugccccuuccacgagguguucaaugccaccaccuucgccucuguguacgccuggaaccgg |
| | | aagcggaucagcaauugcguggccgacuacuccgugaucuacaacuucgcccccuucuucgcauucaagugcuacggcguguccc |
| | | cuaccaagcugaacgaccugugcuucacaaacguguacgccgacagcuucgugauccggggaaacgaagugucacagauugcccc |
| | | uggacagacaggcaacaucgccgacuacaacuacaagcugcccgacgacuucaccggcugugugauugccuggaacagcaacaag |
| | | cuggacuccaaacccagcggcaacuacaauuaccuguaccggcuguuccggaaguccaagcugaagcccuucgagcgggacaucu |
| | | ccaccgagaucuaucaggccggcaacaagccuuguaacggcguggcaggccccaacugcuacagcccacugcaguccuacggcuuu |
| | | aggcccacauacggcgugggccaccagcccuacagagugguggugcugagcuucgaacugcugcaugccucagccacagugugcg |
| | | gcccuaagaaaagcaccaaucucgugaagaacaaaugcgugaacuucaacuucaacggccugaccggcaccggcgugcugacaga |
| | | gagcaacaagaaguuccugccauuccagcaguuuggccgggauaucgccgauaccacagacgccguuagagauccccagacacug |
| | | gaaauccuggacaucaccccuugcagcuucggcggagugucugugaucaccccuggcaccaacaccagcaaucagguggcagugc |
| | | uguaccagggcgugaacuguaccgaagugcccguggccauucacgccgaucagcugacaccuacauggggguguacuccaccgg |
| | | cagcaauguguuucagaccagagccggcugucugaucggagccgaguacgugaacaauagcuacgagugcgacauccccaucggc |
| | | gcuggaaucugcgccagcuaccagacacagacaaagagccaccggagagccagaagcguggccagccagagcaucauugccuacac |
| | | aaugucucugggcgccgagaacagcguggccuacuccaacaacucuaucgcuauccccaccaacuucaccaucagcgugaccacag |
| | | agauccugccuguguccaugaccaagaccagcguggacugcaccauguacaucugcggcgauuccaccgagugcuccaaccugcu |
| | | gcugcaguacggcagcuucugcacccagcugaaaagagcccugacagggaucgccguggaacaggacaagaacacccaagaggug |
| | | uucgcccaagugaagcagaucuacaagaccccuccuaucaaguacuucggcggcuucaauuucagccagauucugcccgauccua |
| | | gcaagcccagcaagcggagcuucaucgaggaccugcuguucaacaaagugacacuggccgacgccggcuucaucaagcaguaugg |
| | | cgauugucugggcgacauugccgccagggaucugauuugcgcccagaaguuuaacggacugacagugcugccuccucugcugac |
| | | cgaugagaugaucgcccaguacacaucugcccugcuggccggcacaaucacaagcggcuggacauuuggagcaggcgccgcucug |
| | | cagauccccuuugcuaugcagauggccuaccgguucaacggcaucggagugacccagaaugugcuguacgagaaccagaagcug |
| | | aucgccaaccaguucaacagcgccaucggcaagauccaggacagccugagcagcacagcaagcgcccugggaaagcugcaggacg |
| | | uggucaaccacaaugcccaggcacugaacacccuggucaagcagcuguccuccaaguucggcgccaucagcucugugcugaacga |
| | | uauccugagcagacuggacccuccugaggccgaggugcagaucgacagacugaucacaggcagacugcagagccuccagacauac |
| | | gugacccagcagcugaucagagccgccgagauuagagccucugccaaucuggccgccaccaagaugucugagugugugcugggcc |
| | | agagcaagagaguggacuuuugcggcaagggcuaccaccugaugagcuucccucagucugccccucacggcgugguguuucugc |
| | | acgugacauaugugcccgcucaagagaagaauuucaccaccgcuccagccaucugccacgacggcaaagcccacuuuccuagagaa |
| | | ggcguguucguguccaacggcacccauugguucgugacacagcggaacuucuacgagccccagaucaucaccaccgacaacaccu |
| | | ucgugucuggcaacugcgacgucgugaucggcauugugaacaauaccguguacgacccucugcagcccgagcuggacagcuucaa |
| | | agaggaacuggacaaguacuuuaagaaccacacaagccccgacguggaccugggcgauaucagcggaaucaaugccagcgucgu |
| | | gaacauccagaaagagaucgaccggcugaacgagguggccaagaaucugaacgagagccugaucgaccugcaagaacuggggaa |
| | | guacgagcaguacaucaaguggcccugguacaucuggcugggcuuuaucgccggacugauugccaucgugauggucacaaucau |
| | | gcuguguugcaugaccagcugcuguagcugccugaagggcuguuguagcuguggcagcugcugcaaguucgacgaggacgauu |
| | | cugagcccgugcugaagggcgugaaacugcacuacacaugaugacucgagcugguacugcaugcacgcaaugcuagcugccccuu |
| | | ucccguccuggguaccccgagucucccccgaccucgggucccagguaugcucccaccuccaccugccccacucaccaccucugcuag |
| | | uuccagacaccucccaagcacgcagcaaugcagcucaaaacgcuuagccuagccacacccccacgggaaacagcagugauuaaccu |
| | | uuagcaauaaacgaaaguuuaacuaagcuauacuaaccccaggguuggucaauuucgugccagccacacccuggagcuagcaaa |
| | | aaaaaaaaaaaaaaaaaaaaaaaaaaagcauaugacuaaaaaaaaaaaaaaaaaaaaaaaaaaaaaaaaaaaaaaaaaaaaaaa |
| | | aaaaaaaaaaaaaaaaaaaaaa |
| 143 | Full length DNA construct encoding a | Agaataaactagtattcttctggtccccacagactcagagagaacccgccaccatgttcgtgttcctggtgctgctgcctctggtgtccagccag |
| | SARS-COV-2 S protein from an | tgtgtgaacctgatcaccagaacacagtcatacaccaacagctttaccagaggcgtgtactaccccgacaaggtgttcagatccagcgtgctg |
| | Omicron XBB.2.3 variant | cactctacccaggacctgttcctgcctttcttcagcaacgtgacctggttccacgccatccacgtgtccggcaccaatggcaccaagagattcga |
| | | caaccccgccctgcccttcaacgacggggtgtactttgccagcaccgagaagtccaacatcatcagaggctggatcttcggcaccacactgga |
| | | cagcaagacccagagcctgctgatcgtgaacaacgccaccaacgtggtcatcaaagtgtgcgagttccagttctgcaacgaccccttcctgga |
| | | cgtctaccagaagaacaacaagagctggatggaaagcgagttccgggtgtacagcagcgccaacaactgcaccttcgagtacgtgtcccag |
| | | cctttcctgatggacctggaaggcaaggagggcaacttcaagaacctgcgcgagttcgtgtttaagaacatcgacggctacttcaagatctac |
| | | agcaagcacacccctatcaacctcgagcgggatctgcctcagggcttctctgctctggaacccctggtggatctgcccatcggcatcaacatca |
| | | cccggtttcagacactgctggccctgcacagaagctacctgacacctggcggcagcagcagcggatggacagctggtgccgccgcttactat |
| | | gtgggctacctgcagcctagaaccttcctgctgaagtacaacgagaacggcaccatcaccgacgccgtggattgtgctctggatcctctgagc |
| | | gagacaaagtgcaccctgaagtccttcaccgtggaaaagggcatctaccagaccagcaacttccgggtgcagcccaccgaatccatcgtgc |
| | | ggttccccaatatcaccaatctgtgccccttccacgaggtgttcaatgccaccaccttcgcctctgtgtacgcctggaaccggaagcggatcagc |
| | | aattgcgtggccgactactccgtgatctacaacttcgcccccttcttcgcattcaagtgctacggcgtgtcccctaccaagctgaacgacctgtg |
| | | cttcacaaacgtgtacgccgacagcttcgtgatccggggaaacgaagtgtcacagattgcccctggacagacaggcaacatcgccgactaca |
| | | actacaagctgcccgacgacttcaccggctgtgtgattgcctggaacagcaacaagctggactccaaacccagcggcaactacaattacctgt |
| | | accggctgttccggaagtccaagctgaagcccttcgagcgggacatctccaccgagatctatcaggccggcaacaagccttgtaacggcgtg |
| | | gcaggccccaactgctacagcccactgcagtcctacggctttaggcccacatacggcgtgggccaccagccctacagagtggtggtgctgag |
| | | cttcgaactgctgcatgcctcagccacagtgtgcggccctaagaaaagcaccaatctcgtgaagaacaaatgcgtgaacttcaacttcaacgg |
| | | cctgaccggcaccggcgtgctgacagagagcaacaagaagttcctgccattccagcagtttggccgggatatcgccgataccacagacgcc |
| | | gttagagatccccagacactggaaatcctggacatcaccccttgcagcttcggcggagtgtctgtgatcacccctggcaccaacaccagcaat |
| | | caggtggcagtgctgtaccagggcgtgaactgtaccgaagtgcccgtggccattcacgccgatcagctgacacctacatggcgggtgtactc |
| | | caccggcagcaatgtgtttcagaccagagccggctgtctgatcggagccgagtacgtgaacaatagctacgagtgcgacatccccatcggcg |
| | | ctggaatctgcgccagctaccagacacagacaaagagccaccggagagccagaagcgtggccagccagagcatcattgcctacacaatgt |
| | | ctctgggcgccgagaacagcgtggcctactccaacaactctatcgctatccccaccaacttcaccatcagcgtgaccacagagatcctgcctgt |
| | | gtccatgaccaagaccagcgtggactgcaccatgtacatctgcggcgattccaccgagtgctccaacctgctgctgcagtacggcagcttctg |
| | | cacccagctgaaaagagccctgacagggatcgccgtggaacaggacaagaacacccaagaggtgttcgcccaagtgaagcagatctaca |
| | | agacccctcctatcaagtacttcggcggcttcaatttcagccagattctgcccgatcctagcaagcccagcaagcggagcttcatcgaggacct |
| | | gctgttcaacaaagtgacactggccgacgccggcttcatcaagcagtatggcgattgtctgggcgacattgccgccagggatctgatttgcgc |
| | | ccagaagtttaacggactgacagtgctgcctcctctgctgaccgatgagatgatcgcccagtacacatctgccctgctggccggcacaatcac |
| | | aagcggctggacatttggagcaggcgccgctctgcagatcccctttgctatgcagatggcctaccggttcaacggcatcggagtgacccaga |
| | | atgtgctgtacgagaaccagaagctgatcgccaaccagttcaacagcgccatcggcaagatccaggacagcctgagcagcacagcaagcg |
| | | ccctgggaaagctgcaggacgtggtcaaccacaatgcccaggcactgaacaccctggtcaagcagctgtcctccaagttcggcgccatcagc |
| | | tctgtgctgaacgatatcctgagcagactggaccctcctgaggccgaggtgcagatcgacagactgatcacaggcagactgcagagcctcca |
| | | gacatacgtgacccagcagctgatcagagccgccgagattagagcctctgccaatctggccgccaccaagatgtctgagtgtgtgctgggcc |
| | | agagcaagagagtggacttttgcggcaagggctaccacctgatgagcttccctcagtctgcccctcacggcgtggtgtttctgcacgtgacata |
| | | tgtgcccgctcaagagaagaatttcaccaccgctccagccatctgccacgacggcaaagcccactttcctagagaaggcgtgttcgtgtccaa |
| | | cggcacccattggttcgtgacacagcggaacttctacgagccccagatcatcaccaccgacaacaccttcgtgtctggcaactgcgacgtcgt |
| | | gatcggcattgtgaacaataccgtgtacgaccctctgcagcccgagctggacagcttcaaagaggaactggacaagtactttaagaaccaca |
| | | caagccccgacgtggacctgggcgatatcagcggaatcaatgccagcgtcgtgaacatccagaaagagatcgaccggctgaacgaggtgg |
| | | ccaagaatctgaacgagagcctgatcgacctgcaagaactggggaagtacgagcagtacatcaagtggccctggtacatctggctgggcttt |
| | | atcgccggactgattgccatcgtgatggtcacaatcatgctgtgttgcatgaccagctgctgtagctgcctgaagggctgttgtagctgtggca |
| | | gctgctgcaagttcgacgaggacgattctgagcccgtgctgaagggcgtgaaactgcactacacatgatgactcgagctggtactgcatgca |
| | | cgcaatgctagctgcccctttcccgtcctgggtaccccgagtctcccccgacctcgggtcccaggtatgctcccacctccacctgccccactcac |
| | | acctctgctagttccagacacctcccaagcacgcagcaatgcagctcaaaacgcttagcctagccacacccccacgggaaacagcagtgatt |
| | | caacctttagcaataaacgaaagtttaactaagctatactaaccccagggttggtcaatttcgtgccagccacaccctggagctagcaaaaaaa |
| | | aaaaaaaaaaaaaaaaa |

TABLE 16D
Sequence of one embodiment of an exemplary Omicron XBB.2.3.2-specific RNA vaccine

| SEQ ID NO. | Brief Description | Sequence |
|---|---|---|
| 144 | Amino acid sequence of RNA-encoded | MFVFLVLLPLVSSQCVNLITRTQSYTNSFTRGVYYPDKVFRSSVLHSTQDLFLPFFSNVTWFHAIHVSGTNGTKR |
| | SARS-COV-2 S protein from an | FDNPALPFNDGVYFASTEKSNIIRGWIFGTTLDSKTQSLLIVNNATNVVIKVCEFQFCNDPFLDVYQKNNKSWM |
| | Omicron XBB.2.3.2 variant (with PRO | ESEFRVYSSANNCTFEYVSQPFLMDLEGKEVNFKNLREFVFKNIDGYFKIYSKHTPINLERDLPQGFSALEPLVDL |
| | mutations at positions corresponding | PIGINITRFQTLLALHRSYLTPGGSSSGWTAGAAAYYVGYLQPRTFLLKYNENGTITDAVDCALDPLSETKCTLKS |
| | to K986P and V987P of SEQ ID NO: 1; | FTVEKGIYQTSNFRVQPTESIVRFPNITNLCPFHEVFNATTFASVYAWNRKRISNCVADYSVIYNFAPFFAFKCYG |
| | i.e., PRO mutations at positions 982 | VSPTKLNDLCFTNVYADSFVIRGNEVSQIAPGQTGNIADYNYKLPDDFTGCVIAWNSNKLDSKPSGNYNYLYRL |
| | and 983 of SEQ ID NO: 144) | FRKSKLKPFERDISTEIYQAGNKPCNGVAGPNCYSPLQSYGFRPTYGVGHQPYRVVVLSFELLHASATVCGPKKS |
| | | TNLVKNKCVNFNFNGLTGTGVLTESNKKFLPFQQFGRDIADTTDAVRDPQTLEILDITPCSFGGVSVITPGTNTS |
| | | NQVAVLYQGVNCTEVPVAIHADQLTPTWRVYSTGSNVFQTRAGCLIGAEYVNNSYECDIPIGAGICASYQTQTK |
| | | SHRRARSVASQSIIAYTMSLGAENSVAYSNNSIAIPTNFTISVTTEILPVSMTKTSVDCTMYICGDSTECSNLLLQ |
| | | YGSFCTQLKRALTGIAVEQDKNTQEVFAQVKQIYKTPPIKYFGGFNFSQILPDPSKPSKRSFIEDLLFNKVTLADA |
| | | GFIKQYGDCLGDIAARDLICAQKFNGLTVLPPLLTDEMIAQYTSALLAGTITSGWTFGAGAALQIPFAMQMAYRF |
| | | NGIGVTQNVLYENQKLIANQFNSAIGKIQDSLSSTASALGKLQDVVNHNAQALNTLVKQLSSKFGAISSVLNDIL |
| | | SRLDPPEAEVQIDRLITGRLQSLQTYVTQQLIRAAEIRASANLAATKMSECVLGQSKRVDFCGKGYHLMSFPQSA |
| | | PHGVVFLHVTYVPAQEKNFTTAPAICHDGKAHFPREGVFVSNGTHWFVTQRNFYEPQIITTDNTFVSGNCDVVI |
| | | GIVNNTVYDPLQPELDSFKEELDKYFKNHTSPDVDLGDISGINASVVNIQKEIDRLNEVAKNLNESLIDLQELGKY |
| | | EQYIKWPWYIWLGFIAGLIAIVMVTIMLCCMTSCCSCLKGCCSCGSCCKFDEDDSEPVLKGVKLHYT |
| 145 | RNA sequence encoding a SARS-COV-2 | auguucguguuccuggugcugcugccucugguguccagccagugugugaaccugaucaccagaacacagucauacaccaacagc |
| | S protein from an Omicron XBB.2.3.2 | uuuaccagaggcguguacuaccccgacaagguguucagauccagcgugcugcacucuacccaggaccuguuccugccuuucuuca |
| | variant | gcaacgugaccugguuccacgccauccacguguccggcaccaauggcaccaagagauucgacaaccccgcccugcccuucaacgac |
| | | gggguguacuuugccagcaccgagaaguccaacaucaucagaggcuggaucuucggcaccacacuggacagcaagacccagagcc |
| | | ugcugaucgugaacaacgccaccaacguggucaucaaagugugcgaguuccaguucugcaacgaccccuuccuggacgucuacca |
| | | gaagaacaacaagagcuggauggaaagcgaguuccggguguacagcagcgccaacaacugcaccuucgaguacgugucccagccu |
| | | uuccugauggaccuggaaggcaaggaggugaacuucaagaaccugcgcgaguucguguuuaagaacaucgacggcuacuucaag |
| | | aucuacagcaagcacaccccuaucaaccucgagcgggaucugccucagggcuucucugcucuggaaccccugguggaucugccca |
| | | ucggcaucaacaucacccgguuucagacacugcuggcccugcacagaagcuaccugacaccuggcggcagcagcagcggauggac |
| | | agcuggugccgccgcuuacuaugugggcuaccugcagccuagaaccuuccugcugaaguacaacgagaacggcaccaucaccgac |
| | | gccguggauugugcucuggauccucugagcgagacaaagugcacccugaaguccuucaccguggaaaagggcaucuaccagacca |
| | | gcaacuuccgggugcagcccaccgaauccaucgugcgguuccccaauaucaccaaucugugccccuuccacgagguguucaaugcc |
| | | accaccuucgccucuguguacgccuggaaccggaagcggaucagcaauugcguggccgacuacuccgugaucuacaacuucgccc |
| | | ccuucuucgcauucaagugcuacggcguguccccuaccaagcugaacgaccugugcuucacaaacguguacgccgacagcuucgu |
| | | gauccggggaaacgaagugucacagauugccccuggacagacaggcaacaucgccgacuacaacuacaagcugcccgacgacuuc |
| | | accggcugugugauugccuggaacagcaacaagcuggacuccaaacccagcggcaacuacaauuaccuguaccggcuguuccgga |
| | | aguccaagcugaagcccuucgagcgggacaucuccaccgagaucuaucaggccggcaacaagccuuguaacggcguggcaggccc |
| | | caacugcuacagcccacugcaguccuacggcuuuaggcccacauacggcgugggccaccagcccuacagagugguggugcugagc |
| | | uucgaacugcugcaugccucagccacagugugcggcccuaagaaaagcaccaaucucgugaagaacaaaugcgugaacuucaacu |
| | | ucaacggccugaccggcaccggcgugcugacagagagcaacaagaaguuccugccauuccagcaguuuggccgggauaucgccga |
| | | uaccacagacgccguuagagauccccagacacuggaaauccuggacaucaccccuugcagcuucggcggagugucugugaucacc |
| | | ccuggcaccaacaccagcaaucagguggcagugcuguaccagggcgugaacuguaccgaagugcccguggccauucacgccgauc |
| | | agcugacaccuacauggcggguguacuccaccggcagcaauguguuucagaccagagccggcugucugaucggagccgaguacg |
| | | ugaacaauagcuacgagugcgacauccccaucggcgcuggaaucugcgccagcuaccagacacagacaaagagccaccggagagc |
| | | cagaagcguggccagccagagcaucauugccuacacaaugucucugggcgccgagaacagcguggccuacuccaacaacucuauc |
| | | gcuauccccaccaacuucaccaucagcgugaccacagagauccugccuguguccaugaccaagaccagcguggacugcaccaugua |
| | | caucugcggcgauuccaccgagugcuccaaccugcugcugcaguacggcagcuucugcacccagcugaaaagagcccugacaggg |
| | | aucgccguggaacaggacaagaacacccaagagguguucgcccaagugaagcagaucuacaagaccccuccuaucaaguacuucg |
| | | gcggcuucaauuucagccagauucugcccgauccuagcaagcccagcaagcggagcuucaucgaggaccugcuguucaacaaagu |
| | | gacacuggccgacgccggcuucaucaagcaguauggcgauugucugggcgacauugccgccagggaucugauuugcgcccagaa |
| | | guuuaacggacugacagugcugccuccucugcugaccgaugagaugaucgcccaguacacaucugcccugcuggccggcacaauc |
| | | acaagcggcuggacauuuggagcaggcgccgcucugcagauccccuuugcuaugcagauggccuaccgguucaacggcaucgga |
| | | gugacccagaaugugcuguacgagaaccagaagcugaucgccaaccaguucaacagcgccaucggcaagauccaggacagccuga |
| | | gcagcacagcaagcgcccugggaaagcugcaggacguggucaaccacaaugcccaggcacugaacacccuggucaagcagcugucc |
| | | uccaaguucggcgccaucagcucugugcugaacgauauccugagcagacuggacccuccugaggccgaggugcagaucgacagac |
| | | ugaucacaggcagacugcagagccuccagacauacgugacccagcagcugaucagagccgccgagauuagagccucugccaaucu |
| | | ggccgccaccaagaugucugagugugugcugggccagagcaagagaguggacuuuugcggcaagggcuaccaccugaugagcuu |
| | | cccucagucugccccucacggcgugguguuucugcacgugacauaugugcccgcucaagagaagaauuucaccaccgcuccagcc |
| | | aucugccacgacggcaaagcccacuuuccuagagaaggcguguucguguccaacggcacccauugguucgugacacagcggaacu |
| | | ucuacgagccccagaucaucaccaccgacaacaccuucgugucuggcaacugcgacgucgugaucggcauugugaacaauaccgu |
| | | guacgacccucugcagcccgagcuggacagcuucaaagaggaacuggacaaguacuuuaagaaccacacaagccccgacguggac |
| | | cugggcgauaucagcggaaucaaugccagcgucgugaacauccagaaagagaucgaccggcugaacgagguggccaagaaucug |
| | | aacgagagccugaucgaccugcaagaacuggggaaguacgagcaguacaucaaguggcccugguacaucuggcugggcuuuauc |
| | | gccggacugauugccaucgugauggucacaaucaugcuguguugcaugaccagcugcuguagcugccugaagggcuguuguagc |
| | | uguggcagcugcugcaaguucgacgaggacgauucugagcccgugcugaagggcgugaaacugcacuacacaugauga |
| 146 | DNA sequence encoding a SARS-COV-2 | atgttcgtgttcctggtgctgctgcctctggtgtccagccagtgtgtgaacctgatcaccagaacacagtcatacaccaacagctttaccagagg |
| | S protein from an Omicron XBB.2.3.2 | cgtgtactaccccgacaaggtgttcagatccagcgtgctgcactctacccaggacctgttcctgcctttcttcagcaacgtgacctggttccacg |
| | variant | ccatccacgtgtccggcaccaatggcaccaagagattcgacaaccccgccctgcccttcaacgacggggtgtactttgccagcaccgagaagt |
| | | ccaacatcatcagaggctggatcttcggcaccacactggacagcaagacccagagcctgctgatcgtgaacaacgccaccaacgtggtcatc |
| | | aaagtgtgcgagttccagttctgcaacgaccccttcctggacgtctaccagaagaacaacaagagctggatggaaagcgagttccgggtgta |
| | | cagcagcgccaacaactgcaccttcgagtacgtgtcccagcctttcctgatggacctggaaggcaaggaggtgaacttcaagaacctgcgcg |
| | | agttcgtgtttaagaacatcgacggctacttcaagatctacagcaagcacacccctatcaacctcgagcgggatctgcctcagggcttctctgct |
| | | ctggaacccctggtggatctgcccatcggcatcaacatcacccggtttcagacactgctggccctgcacagaagctacctgacacctggcggc |
| | | agcagcagcggatggacagctggtgccgccgcttactatgtgggctacctgcagcctagaaccttcctgctgaagtacaacgagaacggcac |
| | | catcaccgacgccgtggattgtgctctggatcctctgagcgagacaaagtgcaccctgaagtccttcaccgtggaaaagggcatctaccagac |
| | | cagcaacttccgggtgcagcccaccgaatccatcgtgcggttccccaatatcaccaatctgtgccccttccacgaggtgttcaatgccaccacct |
| | | tcgcctctgtgtacgcctggaaccggaagcggatcagcaattgcgtggccgactactccgtgatctacaacttcgcccccttcttcgcattcaag |
| | | tgctacggcgtgtcccctaccaagctgaacgacctgtgcttcacaaacgtgtacgccgacagcttcgtgatccggggaaacgaagtgtcaca |
| | | gattgcccctggacagacaggcaacatcgccgactacaactacaagctgcccgacgacttcaccggctgtgtgattgcctggaacagcaaca |
| | | agctggactccaaacccagcggcaactacaattacctgtaccggctgttccggaagtccaagctgaagcccttcgagcgggacatctccacc |
| | | gagatctatcaggccggcaacaagccttgtaacggcgtggcaggccccaactgctacagcccactgcagtcctacggctttaggcccacata |
| | | cggcgtgggccaccagccctacagagtggtggtgctgagcttcgaactgctgcatgcctcagccacagtgtgcggccctaagaaaagcacc |
| | | aatctcgtgaagaacaaatgcgtgaacttcaacttcaacggcctgaccggcaccggcgtgctgacagagagcaacaagaagttcctgccatt |
| | | ccagcagtttggccgggatatcgccgataccacagacgccgttagagatccccagacactggaaatcctggacatcaccccttgcagcttcgg |
| | | cggagtgtctgtgatcacccctggcaccaacaccagcaatcaggtggcagtgctgtaccagggcgtgaactgtaccgaagtgcccgtggcca |
| | | ttcacgccgatcagctgacacctacatggcgggtgtactccaccggcagcaatgtgtttcagaccagagccggctgtctgatcggagccgagt |
| | | acgtgaacaatagctacgagtgcgacatccccatcggcgctggaatctgcgccagctaccagacacagacaaagagccaccggagagcca |
| | | gaagcgtggccagccagagcatcattgcctacacaatgtctctgggcgccgagaacagcgtggcctactccaacaactctatcgctatcccca |
| | | ccaacttcaccatcagcgtgaccacagagatcctgcctgtgtccatgaccaagaccagcgtggactgcaccatgtacatctgcggcgattcca |
| | | ccgagtgctccaacctgctgctgcagtacggcagcttctgcacccagctgaaaagagccctgacagggatcgccgtggaacaggacaagaa |
| | | cacccaagaggtgttcgcccaagtgaagcagatctacaagacccctcctatcaagtacttcggcggcttcaatttcagccagattctgcccgat |
| | | cctagcaagcccagcaagcggagcttcatcgaggacctgctgttcaacaaagtgacactggccgacgccggcttcatcaagcagtatggcg |
| | | attgtctgggcgacattgccgccagggatctgatttgcgcccagaagtttaacggactgacagtgctgcctcctctgctgaccgatgagatgat |
| | | cgcccagtacacatctgccctgctggccggcacaatcacaagcggctggacatttggagcaggcgccgctctgcagatcccctttgctatgca |
| | | gatggcctaccggttcaacggcatcggagtgacccagaatgtgctgtacgagaaccagaagctgatcgccaaccagttcaacagcgccatc |
| | | ggcaagatccaggacagcctgagcagcacagcaagcgccctgggaaagctgcaggacgtggtcaaccacaatgcccaggcactgaacac |
| | | cctggtcaagcagctgtcctccaagttcggcgccatcagctctgtgctgaacgatatcctgagcagactggaccctcctgaggccgaggtgca |
| | | gatcgacagactgatcacaggcagactgcagagcctccagacatacgtgacccagcagctgatcagagccgccgagattagagcctctgcc |
| | | aatctggccgccaccaagatgtctgagtgtgtgctgggccagagcaagagagtggacttttgcggcaagggctaccacctgatgagcttccct |
| | | cagtctgcccctcacggcgtggtgtttctgcacgtgacatatgtgcccgctcaagagaagaatttcaccaccgctccagccatctgccacgacg |
| | | gcaaagcccactttcctagagaaggcgtgttcgtgtccaacggcacccattggttcgtgacacagcggaacttctacgagccccagatcatca |
| | | ccaccgacaacaccttcgtgtctggcaactgcgacgtcgtgatcggcattgtgaacaataccgtgtacgaccctctgcagcccgagctggaca |
| | | gcttcaaagaggaactggacaagtactttaagaaccacacaagccccgacgtggacctgggcgatatcagcggaatcaatgccagcgtcgt |
| | | gaacatccagaaagagatcgaccggctgaacgaggtggccaagaatctgaacgagagcctgatcgacctgcaagaactggggaagtacg |
| | | agcagtacatcaagtggccctggtacatctggctgggctttatcgccggactgattgccatcgtgatggtcacaatcatgctgtgttgcatgacc |
| | | agctgctgtagctgcctgaagggctgttgtagctgtggcagctgctgcaagttcgacgaggacgattctgagcccgtgctgaagggcgtgaa |
| | | actgcactacacatgatga |
| 147 | Full length RNA construct encoding a | Agaauaaacuaguauucuucugguccccacagacucagagagaacccgccaccauguucguguuccuggugcugcugccucugg |
| | SARS-COV-2 S protein from an | uguccagccagugugugaaccugaucaccagaacacagucauacaccaacagcuuuaccagaggcguguacuaccccgacaaggu |
| | Omicron XBB.2.3.2 variant | guucagauccagcgugcugcacucuacccaggaccuguuccugccuuucuucagcaacgugaccugguuccacgccauccacgug |
| | | uccggcaccaauggcaccaagagauucgacaaccccgcccugcccuucaacgacgggguguacuuugccagcaccgagaaguccaa |
| | | caucaucagaggcuggaucuucggcaccacacuggacagcaagacccagagccugcugaucgugaacaacgccaccaacgugguc |
| | | aucaaagugugcgaguuccaguucugcaacgaccccuuccuggacgucuaccagaagaacaacaagagcuggauggaaagcgag |
| | | uuccggguguacagcagcgccaacaacugcaccuucgaguacgugucccagccuuuccugauggaccuggaaggcaaggaggug |
| | | aacuucaagaaccugcgcgaguucguguuuaagaacaucgacggcuacuucaagaucuacagcaagcacaccccuaucaaccucg |
| | | agcgggaucugccucagggcuucucugcucuggaaccccugguggaucugcccaucggcaucaacaucacccgguuucagacacu |
| | | gcuggcccugcacagaagcuaccugacaccuggcggcagcagcagcggauggacagcuggugccgccgcuuacuaugugggcuac |
| | | cugcagccuagaaccuuccugcugaaguacaacgagaacggcaccaucaccgacgccguggauugugcucuggauccucugagcg |
| | | agacaaagugcacccugaaguccuucaccguggaaaagggcaucuaccagaccagcaacuuccgggugcagcccaccgaauccauc |
| | | gugcgguuccccaauaucaccaaucugugccccuuccacgagguguucaaugccaccaccuucgccucuguguacgccuggaaccg |
| | | gaagcggaucagcaauugcguggccgacuacuccgugaucuacaacuucgcccccuucuucgcauucaagugcuacggcgugucc |
| | | ccuaccaagcugaacgaccugugcuucacaaacguguacgccgacagcuucgugauccggggaaacgaagugucacagauugccc |
| | | cuggacagacaggcaacaucgccgacuacaacuacaagcugcccgacgacuucaccggcugugugauugccuggaacagcaacaa |
| | | gcuggacuccaaacccagcggcaacuacaauuaccuguaccggcuguuccggaaguccaagcugaagcccuucgagcgggacauc |
| | | uccaccgagaucuaucaggccggcaacaagccuuguaacggcguggcaggccccaacugcuacagcccacugcaguccuacggcuu |
| | | uaggcccacauacggcgugggccaccagcccuacagagugguggugcugagcuucgaacugcugcaugccucagccacagugugc |
| | | ggcccuaagaaaagcaccaaucucgugaagaacaaaugcgugaacuucaacuucaacggccugaccggcaccggcgugcugacag |
| | | agagcaacaagaaguuccugccauuccagcaguuuggccgggauaucgccgauaccacagacgccguuagagauccccagacacu |
| | | ggaaauccuggacaucaccccuugcagcuucggcggagugucugugaucaccccuggcaccaacaccagcaaucagguggcagug |
| | | cuguaccagggcgugaacuguaccgaagugcccguggccauucacgccgaucagcugacaccuacauggcggguguacuccaccg |
| | | gcagcaauguguuucagaccagagccggcugucugaucggagccgaguacgugaacaauagcuacgagugcgacauccccaucg |
| | | gcgcuggaaucugcgccagcuaccagacacagacaaagagccaccggagagccagaagcguggccagccagagcaucauugccua |
| | | cacaaugucucugggcgccgagaacagcguggccuacuccaacaacucuaucgcuauccccaccaacuucaccaucagcgugacca |
| | | cagagauccugccuguguccaugaccaagaccagcguggacugcaccauguacaucugcggcgauuccaccgagugcuccaaccu |
| | | gcugcugcaguacggcagcuucugcacccagcugaaaagagcccugacagggaucgccguggaacaggacaagaacacccaagag |
| | | guguucgcccaagugaagcagaucuacaagaccccuccuaucaaguacuucggcggcuucaauuucagccagauucugcccgauc |
| | | cuagcaagcccagcaagcggagcuucaucgaggaccugcuguucaacaaagugacacuggccgacgccggcuucaucaagcagua |
| | | uggcgauugucugggcgacauugccgccagggaucugauuugcgcccagaaguuuaacggacugacagugcugccuccucugcu |
| | | gaccgaugagaugaucgcccaguacacaucugcccugcuggccggcacaaucacaagcggcuggacauuuggagcaggcgccgcu |
| | | cugcagauccccuuugcuaugcagauggccuaccgguucaacggcaucggagugacccagaaugugcuguacgagaaccagaagc |
| | | ugaucgccaaccaguucaacagcgccaucggcaagauccaggacagccugagcagcacagcaagcgcccugggaaagcugcagga |
| | | cguggucaaccacaaugcccaggcacugaacacccuggucaagcagcuguccuccaaguucggcgccaucagcucugugcugaac |
| | | gauauccugagcagacuggacccuccugaggccgaggugcagaucgacagacugaucacaggcagacugcagagccuccagacau |
| | | acgugacccagcagcugaucagagccgccgagauuagagccucugccaaucuggccgccaccaagaugucugagugugugcuggg |
| | | ccagagcaagagaguggacuuuugcggcaagggcuaccaccugaugagcuucccucagucugccccucacggcgugguguuucu |
| | | gcacgugacauaugugcccgcucaagagaagaauuucaccaccgcuccagccaucugccacgacggcaaagcccacuuuccuagag |
| | | aaggcguguucguguccaacggcacccauugguucgugacacagcggaacuucuacgagccccagaucaucaccaccgacaacacc |
| | | uucgugucuggcaacugcgacgucgugaucggcauugugaacaauaccguguacgacccucugcagcccgagcuggacagcuuc |
| | | aaagaggaacuggacaaguacuuuaagaaccacacaagccccgacguggaccugggcgauaucagcggaaucaaugccagcgucg |
| | | ugaacauccagaaagagaucgaccggcugaacgagguggccaagaaucugaacgagagccugaucgaccugcaagaacugggga |
| | | aguacgagcaguacaucaaguggcccugguacaucuggcugggcuuuaucgccggacugauugccaucgugauggucacaauca |
| | | ugcuguguugcaugaccagcugcuguagcugccugaagggcuguuguagcuguggcagcugcugcaaguucgacgaggacgau |
| | | ucugagcccgugcugaagggcgugaaacugcacuacacaugaugacucgagcugguacugcaugcacgcaaugcuagcugccccu |
| | | uucccguccuggguaccccgagucucccccgaccucgggucccagguaugcucccaccuccaccugccccacucaccaccucugcua |
| | | guuccagacaccucccaagcacgcagcaaugcagcucaaaacgcuuagccuagccacacccccacgggaaacagcagugauuaacc |
| | | uuuagcaauaaacgaaaguuuaacuaagcuauacuaaccccaggguuggucaauuucgugccagccacacccuggagcuagcaa |
| | | aaaaaaaaaaaaaaaaaaaaaaaaaaaagcauaugacuaaaaaaaaaaaaaaaaaaaaaaaaaaaaaaaaaaaaaaaaaaaaaa |
| | | aaaaaaaaaaaaaaaaaaaaaaa |
| 148 | Full length DNA construct encoding a | Agaataaactagtattcttctggtccccacagactcagagagaacccgccaccatgttcgtgttcctggtgctgctgcctctggtgtccagccag |
| | SARS-COV-2 S protein from an | tgtgtgaacctgatcaccagaacacagtcatacaccaacagctttaccagaggcgtgtactaccccgacaaggtgttcagatccagcgtgctg |
| | Omicron XBB.2.3.2 variant | cactctacccaggacctgttcctgcctttcttcagcaacgtgacctggttccacgccatccacgtgtccggcaccaatggcaccaagagattcga |
| | | caaccccgccctgcccttcaacgacggggtgtactttgccagcaccgagaagtccaacatcatcagaggctggatcttcggcaccacactgga |
| | | cagcaagacccagagcctgctgatcgtgaacaacgccaccaacgtggtcatcaaagtgtgcgagttccagttctgcaacgaccccttcctgga |
| | | cgtctaccagaagaacaacaagagctggatggaaagcgagttccgggtgtacagcagcgccaacaactgcaccttcgagtacgtgtcccag |
| | | cctttcctgatggacctggaaggcaaggaggtgaacttcaagaacctgcgcgagttcgtgtttaagaacatcgacggctacttcaagatctac |
| | | agcaagcacacccctatcaacctcgagcgggatctgcctcagggcttctctgctctggaacccctggtggatctgcccatcggcatcaacatca |
| | | cccggtttcagacactgctggccctgcacagaagctacctgacacctggcggcagcagcagcggatggacagctggtgccgccgcttactat |
| | | gtgggctacctgcagcctagaaccttcctgctgaagtacaacgagaacggcaccatcaccgacgccgtggattgtgctctggatcctctgagc |
| | | gagacaaagtgcaccctgaagtccttcaccgtggaaaagggcatctaccagaccagcaacttccgggtgcagcccaccgaatccatcgtgc |
| | | ggttccccaatatcaccaatctgtgccccttccacgaggtgttcaatgccaccaccttcgcctctgtgtacgcctggaaccggaagcggatcagc |
| | | aattgcgtggccgactactccgtgatctacaacttcgcccccttcttcgcattcaagtgctacggcgtgtcccctaccaagctgaacgacctgtg |
| | | cttcacaaacgtgtacgccgacagcttcgtgatccggggaaacgaagtgtcacagattgcccctggacagacaggcaacatcgccgactaca |
| | | actacaagctgcccgacgacttcaccggctgtgtgattgcctggaacagcaacaagctggactccaaacccagcggcaactacaattacctgt |
| | | accggctgttccggaagtccaagctgaagcccttcgagcgggacatctccaccgagatctatcaggccggcaacaagccttgtaacggcgtg |
| | | gcaggccccaactgctacagcccactgcagtcctacggctttaggcccacatacggcgtgggccaccagccctacagagtggtggtgctgag |
| | | cttcgaactgctgcatgcctcagccacagtgtgcggccctaagaaaagcaccaatctcgtgaagaacaaatgcgtgaacttcaacttcaacgg |
| | | cctgaccggcaccggcgtgctgacagagagcaacaagaagttcctgccattccagcagtttggccgggatatcgccgataccacagacgcc |
| | | gttagagatccccagacactggaaatcctggacatcaccccttgcagcttcggcggagtgtctgtgatcacccctggcaccaacaccagcaat |
| | | caggtggcagtgctgtaccagggcgtgaactgtaccgaagtgcccgtggccattcacgccgatcagctgacacctacatggcgggtgtactc |
| | | caccggcagcaatgtgtttcagaccagagccggctgtctgatcggagccgagtacgtgaacaatagctacgagtgcgacatccccatcggcg |
| | | ctggaatctgcgccagctaccagacacagacaaagagccaccggagagccagaagcgtggccagccagagcatcattgcctacacaatgt |
| | | ctctgggcgccgagaacagcgtggcctactccaacaactctatcgctatccccaccaacttcaccatcagcgtgaccacagagatcctgcctgt |
| | | gtccatgaccaagaccagcgtggactgcaccatgtacatctgcggcgattccaccgagtgctccaacctgctgctgcagtacggcagcttctg |
| | | cacccagctgaaaagagccctgacagggatcgccgtggaacaggacaagaacacccaagaggtgttcgcccaagtgaagcagatctaca |
| | | agacccctcctatcaagtacttcggcggcttcaatttcagccagattctgcccgatcctagcaagcccagcaagcggagcttcatcgaggacct |
| | | gctgttcaacaaagtgacactggccgacgccggcttcatcaagcagtatggcgattgtctgggcgacattgccgccagggatctgatttgcgc |
| | | ccagaagtttaacggactgacagtgctgcctcctctgctgaccgatgagatgatcgcccagtacacatctgccctgctggccggcacaatcac |
| | | aagcggctggacatttggagcaggcgccgctctgcagatcccctttgctatgcagatggcctaccggttcaacggcatcggagtgacccaga |
| | | atgtgctgtacgagaaccagaagctgatcgccaaccagttcaacagcgccatcggcaagatccaggacagcctgagcagcacagcaagcg |
| | | ccctgggaaagctgcaggacgtggtcaaccacaatgcccaggcactgaacaccctggtcaagcagctgtcctccaagttcggcgccatcagc |
| | | tctgtgctgaacgatatcctgagcagactggaccctcctgaggccgaggtgcagatcgacagactgatcacaggcagactgcagagcctcca |
| | | gacatacgtgacccagcagctgatcagagccgccgagattagagcctctgccaatctggccgccaccaagatgtctgagtgtgtgctgggcc |
| | | agagcaagagagtggacttttgcggcaagggctaccacctgatgagcttccctcagtctgcccctcacggcgtggtgtttctgcacgtgacata |
| | | tgtgcccgctcaagagaagaatttcaccaccgctccagccatctgccacgacggcaaagcccactttcctagagaaggcgtgttcgtgtccaa |
| | | cggcacccattggttcgtgacacagcggaacttctacgagccccagatcatcaccaccgacaacaccttcgtgtctggcaactgcgacgtcgt |
| | | gatcggcattgtgaacaataccgtgtacgaccctctgcagcccgagctggacagcttcaaagaggaactggacaagtactttaagaaccaca |
| | | caagccccgacgtggacctgggcgatatcagcggaatcaatgccagcgtcgtgaacatccagaaagagatcgaccggctgaacgaggtgg |
| | | ccaagaatctgaacgagagcctgatcgacctgcaagaactggggaagtacgagcagtacatcaagtggccctggtacatctggctgggcttt |
| | | atcgccggactgattgccatcgtgatggtcacaatcatgctgtgttgcatgaccagctgctgtagctgcctgaagggctgttgtagctgtggca |
| | | gctgctgcaagttcgacgaggacgattctgagcccgtgctgaagggcgtgaaactgcactacacatgatgactcgagctggtactgcatgca |
| | | cgcaatgctagctgcccctttcccgtcctgggtaccccgagtctcccccgacctcgggtcccaggtatgctcccacctccacctgccccactcac |
| | | cacctctgctagttccagacacctcccaagcacgcagcaatgcagctcaaaacgcttagcctagccacacccccacgggaaacagcagtgatt |
| | | aacctttagcaataaacgaaagtttaactaagctatactaaccccagggttggtcaatttcgtgccagccacaccctggagctagcaaaaaaa |
| | | aaaaaaaaaaaaaaaaaaaaaaagcatatgactaaaaaaaaaaaaaaaaaaaaaaaaaaaaaaaaaaaaaaaaaaaaaaaaaaaa |
| | | aaaaaaaaaaaaaaaaa |

TABLE 17
Sequence of one embodiment of an A/Darwin/6/2021-specific Influenza RNA vaccine

| SEQ | | |
|---|---|---|
| ID | Brief | |
| NO. | Description | Sequence |
| 80 | Amino acid | MKTIIALSNILCLVFAQKIPGNDNSTATLCLGHHAVPNGTIVKTITNDRIEVTNATELVQNSSIGEICGSPHQ |
| | sequence of | ILDGGNCTLIDALLGDPQCDGFQNKEWDLFVERSRANSNCYPYDVPDYASLRSLVASSGTLEFKNESFNWTGV |
| | RNA-encoded | KQNGTSSACIRGSSSSFFSRLNWLTSLNNIYPAQNVTMPNKEQFDKLYIWGVHHPDTDKNQISLFAQSSGRIT |
| | Influenza HA | VSTKRSQQAVIPNIGSRPRIRDIPSRISIYWTIVKPGDILLINSTGNLIAPRGYFKIRSGKSSIMRSDAPIGK |
| | protein of | CKSECITPNGSIPNDKPFQNVNRITYGACPRYVKQSTLKLATGMRNVPEKQTRGIFGAIAGFIENGWEGMVDG |
| | A/Darwin/ | WYGFRHQNSEGRGQAADLKSTQAAIDQINGKLNRLIGKTNEKFHQIEKEFSEVEGRVQDLEKYVEDTKIDLWS |
| | 6/2021 | YNAELLVALENQHTIDLTDSEMNKLFEKTKKQLRENAEDMGNGCFKIYHKCDNACIGSIRNETYDHNVYRDEA |
| | | LNNRFQIKGVELKSGYKDWILWISFAMSCFLLCIALLGFIMWACQKGNIRCNICI |
| 81 | DNA sequence | atgaagaccatcattgccctgagcaacatcctgtgcctggtgttcgcccagaagatccccggcaacgacaata |
| | encoding an | gcaccgccacactgtgtctgggccatcacgctgtgcctaacggcaccatcgtgaaaaccatcaccaacgaccg |
| | influenza HA | gatcgaagtgaccaacgccacagagctggtgcagaacagcagcatcggcgagatctgtggcagccctcaccag |
| | protein of | attctggacggcggcaattgcaccctgatcgatgctctgctgggcgaccctcagtgtgacggcttccagaaca |
| | A/Darwin/ | aagaatgggacctgttcgtggaaagaagccgggccaacagcaactgctacccctacgatgtgcccgactacgc |
| | 6/2021 | cagcctgagatctctggtggcctctagcggcaccctggaattcaagaacgagagcttcaactggaccggcgtg |
| | | aagcagaatggcaccagcagcgcctgtatcagaggcagcagctccagcttcttcagcagactgaactggctga |
| | | ccagcctgaacaacatctaccccgctcagaacgtgaccatgcctaacaaagagcagttcgacaagctgtacat |
| | | ctggggcgtgcaccatcctgacaccgacaagaaccagatcagcctgtttgcccagagcagcggcagaatcacc |
| | | gtgtccactaagagaagccagcaggccgtgattcccaacatcggcagcagaccccggatcagagacatcccca |
| | | gccggatcagcatctactggacaatcgtgaagcccggcgacatcctgctgatcaacagcaccggcaatctgat |
| | | cgcccctcggggctacttcaagatcagaagcggcaagagcagcatcatgcggagcgacgcccctatcggcaag |
| | | tgcaagagcgagtgcatcaccccaaacggcagcatccccaacgacaagcccttccagaatgtgaaccggatca |
| | | cctacggcgcctgtcctagatacgtgaaacagagcaccctgaagctggccaccggcatgagaaacgtgccaga |
| | | gaagcagaccagaggcatcttcggagccattgccggcttcatcgagaacggctgggaaggcatggtggacgga |
| | | tggtacggcttcagacaccagaacagcgaaggcagaggacaggccgctgacctgaaatctacacaggccgcca |
| | | tcgaccagatcaacggcaagctgaaccggctgatcggcaagaccaacgagaagttccaccagatcgagaaaga |
| | | gttcagcgaggtcgagggcagagtgcaggacctcgagaaatacgtggaagataccaagatcgacctgtggtcc |
| | | tacaatgccgaactgctggtggccctggaaaaccagcacaccatcgacctgaccgacagcgagatgaacaagc |
| | | tgttcgaaaagaccaagaagcagctgcgcgagaacgccgaggatatgggcaacggctgctttaagatctacca |
| | | caagtgcgacaacgcctgcatcggctccatccggaacgagacatacgaccacaacgtgtacagagatgaggcc |
| | | ctgaacaatcggttccagatcaaaggcgtggaactgaagtccggctacaaggactggatcctgtggatcagct |
| | | tcgccatgagctgctttctgctgtgtatcgccctgctgggcttcatcatgtgggcctgccagaaaggcaacat |
| | | cagatgcaacatctgcatctgatga |
| 82 | RNA sequence | augaagaccaucauugcccugagcaacauccugugccugguguucgcccagaagauccccggcaacgacaaua |
| | encoding an | gcaccgccacacugugucugggccaucacgcugugccuaacggcaccaucgugaaaaccaucaccaacgaccg |
| | influenza HA | gaucgaagugaccaacgccacagagcuggugcagaacagcagcaucggcgagaucuguggcagcccucaccag |
| | protein of | auucuggacggcggcaauugcacccugaucgaugcucugcugggcgacccucagugugacggcuuccagaaca |
| | A/Darwin/ | aagaaugggaccuguucguggaaagaagccgggccaacagcaacugcuaccccuacgaugugcccgacuacgc |
| | 6/2021 | cagccugagaucucugguggccucuagcggcacccuggaauucaagaacgagagcuucaacuggaccggcgug |
| | | aagcagaauggcaccagcagcgccuguaucagaggcagcagcuccagcuucuucagcagacugaacuggcuga |
| | | ccagccugaacaacaucuaccccgcucagaacgugaccaugccuaacaaagagcaguucgacaagcuguacau |
| | | cuggggcgugcaccauccugacaccgacaagaaccagaucagccuguuugcccagagcagcggcagaaucacc |
| | | guguccacuaagagaagccagcaggccgugauucccaacaucggcagcagaccccggaucagagacaucccca |
| | | gccggaucagcaucuacuggacaaucgugaagcccggcgacauccugcugaucaacagcaccggcaaucugau |
| | | cgccccucggggcuacuucaagaucagaagcggcaagagcagcaucaugcggagcgacgccccuaucggcaag |
| | | ugcaagagcgagugcaucaccccaaacggcagcauccccaacgacaagcccuuccagaaugugaaccggauca |
| | | ccuacggcgccuguccuagauacgugaaacagagcacccugaagcuggccaccggcaugagaaacgugccaga |
| | | gaagcagaccagaggcaucuucggagccauugccggcuucaucgagaacggcugggaaggcaugguggacgga |
| | | ugguacggcuucagacaccagaacagcgaaggcagaggacaggccgcugaccugaaaucuacacaggccgcca |
| | | ucgaccagaucaacggcaagcugaaccggcugaucggcaagaccaacgagaaguuccaccagaucgagaaaga |
| | | guucagcgaggucgagggcagagugcaggaccucgagaaauacguggaagauaccaagaucgaccuguggucc |
| | | uacaaugccgaacugcugguggcccuggaaaaccagcacaccaucgaccugaccgacagcgagaugaacaagc |
| | | uguucgaaaagaccaagaagcagcugcgcgagaacgccgaggauaugggcaacggcugcuuuaagaucuacca |
| | | caagugcgacaacgccugcaucggcuccauccggaacgagacauacgaccacaacguguacagagaugaggcc |
| | | cugaacaaucgguuccagaucaaaggcguggaacugaaguccggcuacaaggacuggauccuguggaucagcu |
| | | ucgccaugagcugcuuucugcuguguaucgcccugcugggcuucaucaugugggccugccagaaaggcaacau |
| | | cagaugcaacaucugcaucugauga |
| 83 | Full length | agaataaactagtattcttctggtccccacagactcagagagaacccgccaccatgaagaccatcattgccct |
| | DNA | gagcaacatcctgtgcctggtgttcgcccagaagatccccggcaacgacaatagcaccgccacactgtgtctg |
| | construct | ggccatcacgctgtgcctaacggcaccatcgtgaaaaccatcaccaacgaccggatcgaagtgaccaacgcca |
| | sequence | cagagctggtgcagaacagcagcatcggcgagatctgtggcagccctcaccagattctggacggcggcaattg |
| | encoding | caccctgatcgatgctctgctgggcgaccctcagtgtgacggcttccagaacaaagaatgggacctgttcgtg |
| | an HA | gaaagaagccgggccaacagcaactgctacccctacgatgtgcccgactacgccagcctgagatctctggtgg |
| | protein of | cctctagcggcaccctggaattcaagaacgagagcttcaactggaccggcgtgaagcagaatggcaccagcag |
| | A/Darwin/ | cgcctgtatcagaggcagcagctccagcttcttcagcagactgaactggctgaccagcctgaacaacatctac |
| | 6/2021 | cccgctcagaacgtgaccatgcctaacaaagagcagttcgacaagctgtacatctggggcgtgcaccatcctg |
| | | acaccgacaagaaccagatcagcctgtttgcccagagcagcggcagaatcaccgtgtccactaagagaagcca |
| | | gcaggccgtgattcccaacatcggcagcagaccccggatcagagacatccccagccggatcagcatctactgg |
| | | acaatcgtgaagcccggcgacatcctgctgatcaacagcaccggcaatctgatcgcccctcggggctacttca |
| | | agatcagaagcggcaagagcagcatcatgcggagcgacgcccctatcggcaagtgcaagagcgagtgcatcac |
| | | cccaaacggcagcatccccaacgacaagcccttccagaatgtgaaccggatcacctacggcgcctgtcctaga |
| | | tacgtgaaacagagcaccctgaagctggccaccggcatgagaaacgtgccagagaagcagaccagaggcatct |
| | | tcggagccattgccggcttcatcgagaacggctgggaaggcatggtggacggatggtacggcttcagacacca |
| | | gaacagcgaaggcagaggacaggccgctgacctgaaatctacacaggccgccatcgaccagatcaacggcaag |
| | | ctgaaccggctgatcggcaagaccaacgagaagttccaccagatcgagaaagagttcagcgaggtcgagggca |
| | | gagtgcaggacctcgagaaatacgtggaagataccaagatcgacctgtggtcctacaatgccgaactgctggt |
| | | ggccctggaaaaccagcacaccatcgacctgaccgacagcgagatgaacaagctgttcgaaaagaccaagaag |
| | | cagctgcgcgagaacgccgaggatatgggcaacggctgctttaagatctaccacaagtgcgacaacgcctgca |
| | | tcggctccatccggaacgagacatacgaccacaacgtgtacagagatgaggccctgaacaatcggttccagat |
| | | caaaggcgtggaactgaagtccggctacaaggactggatcctgtggatcagcttcgccatgagctgctttctg |
| | | ctgtgtatcgccctgctgggcttcatcatgtgggcctgccagaaaggcaacatcagatgcaacatctgcatct |
| | | gatgactcgagctggtactgcatgcacgcaatgctagctgcccctttcccgtcctgggtaccccgagtctccc |
| | | ccgacctcgggtcccaggtatgctcccacctccacctgccccactcaccacctctgctagttccagacacctc |
| | | ccaagcacgcagcaatgcagctcaaaacgcttagcctagccacacccccacgggaaacagcagtgattaacct |
| | | ttagcaataaacgaaagtttaactaagctatactaaccccagggttggtcaatttcgtgccagccacaccctg |
| | | gagctagcaaaaaaaaaaaaaaaaaaaaaaaaaaaaaagcatatgactaaaaaaaaaaaaaaaaaaaaaaaaa |
| | | aaaaaaaaaaaaaaaaaaaaaaaaaaaaaaaaaaaaaaaaaaaa |
| 84 | Full length | agaauaaacuaguauucuucugguccccacagacucagagagaacccgccaccaugaagaccaucauugcccu |
| | RNA | gagcaacauccugugccugguguucgcccagaagauccccggcaacgacaauagcaccgccacacugugucug |
| | construct | ggccaucacgcugugccuaacggcaccaucgugaaaaccaucaccaacgaccggaucgaagugaccaacgcca |
| | sequence | cagagcuggugcagaacagcagcaucggcgagaucuguggcagcccucaccagauucuggacggcggcaauug |
| | encoding | cacccugaucgaugcucugcugggcgacccucagugugacggcuuccagaacaaagaaugggaccuguucgug |
| | an HA | gaaagaagccgggccaacagcaacugcuaccccuacgaugugcccgacuacgccagccugagaucucuggugg |
| | protein of | ccucuagcggcacccuggaauucaagaacgagagcuucaacuggaccggcgugaagcagaauggcaccagcag |
| | A/Darwin/ | cgccuguaucagaggcagcagcuccagcuucuucagcagacugaacuggcugaccagccugaacaacaucuac |
| | 6/2021 | cccgcucagaacgugaccaugccuaacaaagagcaguucgacaagcuguacaucuggggcgugcaccauccug |
| | | acaccgacaagaaccagaucagccuguuugcccagagcagcggcagaaucaccguguccacuaagagaagcca |
| | | gcaggccgugauucccaacaucggcagcagaccccggaucagagacauccccagccggaucagcaucuacugg |
| | | acaaucgugaagcccggcgacauccugcugaucaacagcaccggcaaucugaucgccccucggggcuacuuca |
| | | agaucagaagcggcaagagcagcaucaugcggagcgacgccccuaucggcaagugcaagagcgagugcaucac |
| | | cccaaacggcagcauccccaacgacaagcccuuccagaaugugaaccggaucaccuacggcgccuguccuaga |
| | | uacgugaaacagagcacccugaagcuggccaccggcaugagaaacgugccagagaagcagaccagaggcaucu |
| | | ucggagccauugccggcuucaucgagaacggcugggaaggcaugguggacggaugguacggcuucagacacca |
| | | gaacagcgaaggcagaggacaggccgcugaccugaaaucuacacaggccgccaucgaccagaucaacggcaag |
| | | cugaaccggcugaucggcaagaccaacgagaaguuccaccagaucgagaaagaguucagcgaggucgagggca |
| | | gagugcaggaccucgagaaauacguggaagauaccaagaucgaccugugguccuacaaugccgaacugcuggu |
| | | ggcccuggaaaaccagcacaccaucgaccugaccgacagcgagaugaacaagcuguucgaaaagaccaagaag |
| | | cagcugcgcgagaacgccgaggauaugggcaacggcugcuuuaagaucuaccacaagugcgacaacgccugca |
| | | ucggcuccauccggaacgagacauacgaccacaacguguacagagaugaggcccugaacaaucgguuccagau |
| | | caaaggcguggaacugaaguccggcuacaaggacuggauccuguggaucagcuucgccaugagcugcuuucug |
| | | cuguguaucgcccugcugggcuucaucaugugggccugccagaaaggcaacaucagaugcaacaucugcaucu |
| | | gaugacucgagcugguacugcaugcacgcaaugcuagcugccccuuucccguccuggguaccccgagucuccc |
| | | ccgaccucgggucccagguaugcucccaccuccaccugccccacucaccaccucugcuaguuccagacaccuc |
| | | ccaagcacgcagcaaugcagcucaaaacgcuuagccuagccacacccccacgggaaacagcagugauuaaccu |
| | | uuagcaauaaacgaaaguuuaacuaagcuauacuaaccccaggguuggucaauuucgugccagccacacccug |
| | | gagcuagcaaaaaaaaaaaaaaaaaaaaaaaaaaaaaagcauaugacuaaaaaaaaaaaaaaaaaaaaaaaaa |
| | | aaaaaaaaaaaaaaaaaaaaaaaaaaaaaaaaaaaaaaaaaaaa |

TABLE 18
Sequence of one embodiment of a B/Austria/1359417/2021-specific Influenza RNA vaccine

| SEQ | | |
|---|---|---|
| ID | Brief | |
| NO. | Description | Sequence |
| 85 | Amino acid | MKAIIVLLMVVTSNADRICTGITSSNSPHVVKTATQGEVNVTGVIPLTTTPTKSHFANLKGTETRGKLCPKCL |
| | sequence of | NCTDLDVALGRPKCTGKIPSARVSILHEVRPVTSGCFPIMHDRTKIRQLPNLLRGYEHVRLSTHNVINTEDAP |
| | RNA-encoded | GGPYEIGTSGSCLNITNGKGFFATMAWAVPKNKTATNPLTIEVPYICTEEEDQITVWGFHSDDETQMARLYGD |
| | Influenza HA | SKPQKFTSSANGVTTHYVSQIGGFPNQTEDGGLPQSGRIVVDYMVQKSGKTGTITYQRGILLPQKVWCASGKS |
| | protein of | KVIKGSLPLIGEADCLHEKYGGLNKSKPYYTGEHAKAIGNCPIWVKTPLKLANGTKYRPPAKLLKERGFFGAI |
| | B/Austria/ | AGFLEGGWEGMIAGWHGYTSHGAHGVAVAADLKSTQEAINKITKNLNSLSELEVKNLQRLSGAMDELHNEILE |
| | 1359417/2021 | LDEKVDDLRADTISSQIELAVLLSNEGIINSEDEHLLALERKLKKMLGPSAVEIGNGCFETKHKCNQTCLDRI |
| | | AAGTFDAGEFSLPTFDSLNITAASLNDDGLDNHTILLYYSTAASSLAVTLMIAIFVVYMVSRDNVSCSICL |
| 86 | DNA sequence | atgaaggccatcatcgtgctgctgatggtggtcaccagcaacgccgacagaatctgcaccggcatcaccagca |
| | encoding an | gcaacagccctcacgtggtcaagacagccacacagggcgaagtgaatgtgaccggcgtgatccctctgaccac |
| | influenza HA | cacacctaccaagagccacttcgccaacctgaagggcaccgagacaagaggcaagctgtgccccaagtgcctg |
| | protein of | aactgcaccgatctggatgtggccctgggcagacctaagtgtaccggcaagatccctagcgccagagtgtcca |
| | B/Austria/ | tcctgcacgaagtgcggcctgtgaccagcggctgctttcccattatgcacgaccggaccaagatcagacagct |
| | 1359417/2021 | gcccaatctgctgcggggctatgaacatgtgcggctgagcacccacaacgtgatcaacacagaggatgcccct |
| | | ggcggcccttacgagatcggaacatctggctcttgcctgaacattaccaacggcaagggcttcttcgccacca |
| | | tggcttgggccgtgcctaagaacaagaccgccaccaatccactgaccatcgaggtgccctacatctgtaccga |
| | | ggaagaagatcagatcaccgtctggggcttccacagcgacgacgaaacacagatggccagactgtacggcgac |
| | | agcaagcctcagaagttcaccagctctgccaacggcgtgaccacacactacgtgtcccagatcggcggcttcc |
| | | ccaatcagacagaagatggcggactgccccagagcggaagaatcgtggtggactacatggtgcagaagtccgg |
| | | caagaccggcacaatcacataccagcggggaatcctgctgcctcagaaagtttggtgcgccagcggcaagagc |
| | | aaagtgatcaagggcagcctgcctctgatcggcgaagccgattgtctgcacgagaagtacggcggcctgaaca |
| | | agagcaagccctactacacaggcgagcacgccaaggccatcggcaactgtcctatctgggtcaagacccctct |
| | | gaagctggccaacggcaccaagtatagacctccagccaagctgctgaaagagcggggcttctttggagctatc |
| | | gccggctttcttgaaggcggctgggagggaatgattgccggctggcatggctacacatctcatggcgcacatg |
| | | gcgtggcagtggccgctgatctgaagtctacacaagaggccatcaacaagatcaccaagaacctgaacagcct |
| | | gagcgagctggaagtgaagaacctgcagagactgtccggcgccatggacgagctgcacaacgagatcctggaa |
| | | ctggacgagaaggtggacgacctgagagccgataccatctccagccagattgagctggcagtgctgctgtcca |
| | | acgagggcatcatcaacagcgaggacgagcatctgctggccctggaacggaagctgaagaagatgctgggccc |
| | | aagcgccgtggaaatcggcaatggctgcttcgagacaaagcacaagtgcaaccagacctgcctggacagaatt |
| | | gccgccggaacatttgacgccggcgagtttagcctgcctaccttcgactccctgaacatcacagccgccagcc |
| | | tgaatgacgacggcctggacaatcacaccatcctgctgtactactccaccgccgcttctagcctggccgtgac |
| | | actgatgatcgccatctttgtggtgtacatggtgtccagagacaacgtgtcctgcagcatctgcctgtgatga |
| 87 | RNA sequence | augaaggccaucaucgugcugcugaugguggucaccagcaacgccgacagaaucugcaccggcaucaccagca |
| | encoding an | gcaacagcccucacguggucaagacagccacacagggcgaagugaaugugaccggcgugaucccucugaccac |
| | influenza HA | cacaccuaccaagagccacuucgccaaccugaagggcaccgagacaagaggcaagcugugccccaagugccug |
| | protein of | aacugcaccgaucuggauguggcccugggcagaccuaaguguaccggcaagaucccuagcgccagagugucca |
| | B/Austria/ | uccugcacgaagugcggccugugaccagcggcugcuuucccauuaugcacgaccggaccaagaucagacagcu |
| | 1359417/2021 | gcccaaucugcugcggggcuaugaacaugugcggcugagcacccacaacgugaucaacacagaggaugccccu |
| | | ggcggcccuuacgagaucggaacaucuggcucuugccugaacauuaccaacggcaagggcuucuucgccacca |
| | | uggcuugggccgugccuaagaacaagaccgccaccaauccacugaccaucgaggugcccuacaucuguaccga |
| | | ggaagaagaucagaucaccgucuggggcuuccacagcgacgacgaaacacagauggccagacuguacggcgac |
| | | agcaagccucagaaguucaccagcucugccaacggcgugaccacacacuacgugucccagaucggcggcuucc |
| | | ccaaucagacagaagauggcggacugccccagagcggaagaaucgugguggacuacauggugcagaaguccgg |
| | | caagaccggcacaaucacauaccagcggggaauccugcugccucagaaaguuuggugcgccagcggcaagagc |
| | | aaagugaucaagggcagccugccucugaucggcgaagccgauugucugcacgagaaguacggcggccugaaca |
| | | agagcaagcccuacuacacaggcgagcacgccaaggccaucggcaacuguccuaucugggucaagaccccucu |
| | | gaagcuggccaacggcaccaaguauagaccuccagccaagcugcugaaagagcggggcuucuuuggagcuauc |
| | | gccggcuuucuugaaggcggcugggagggaaugauugccggcuggcauggcuacacaucucauggcgcacaug |
| | | gcguggcaguggccgcugaucugaagucuacacaagaggccaucaacaagaucaccaagaaccugaacagccu |
| | | gagcgagcuggaagugaagaaccugcagagacuguccggcgccauggacgagcugcacaacgagauccuggaa |
| | | cuggacgagaagguggacgaccugagagccgauaccaucuccagccagauugagcuggcagugcugcugucca |
| | | acgagggcaucaucaacagcgaggacgagcaucugcuggcccuggaacggaagcugaagaagaugcugggccc |
| | | aagcgccguggaaaucggcaauggcugcuucgagacaaagcacaagugcaaccagaccugccuggacagaauu |
| | | gccgccggaacauuugacgccggcgaguuuagccugccuaccuucgacucccugaacaucacagccgccagcc |
| | | ugaaugacgacggccuggacaaucacaccauccugcuguacuacuccaccgccgcuucuagccuggccgugac |
| | | acugaugaucgccaucuuugugguguacaugguguccagagacaacguguccugcagcaucugccugugauga |
| 88 | Full length | agaataaactagtattcttctggtccccacagactcagagagaacccgccaccatgaaggccatcatcgtgct |
| | DNA construct | gctgatggtggtcaccagcaacgccgacagaatctgcaccggcatcaccagcagcaacagccctcacgtggtc |
| | sequence | aagacagccacacagggcgaagtgaatgtgaccggcgtgatccctctgaccaccacacctaccaagagccact |
| | encoding | tcgccaacctgaagggcaccgagacaagaggcaagctgtgccccaagtgcctgaactgcaccgatctggatgt |
| | an HA | ggccctgggcagacctaagtgtaccggcaagatccctagcgccagagtgtccatcctgcacgaagtgcggcct |
| | protein of | gtgaccagcggctgctttcccattatgcacgaccggaccaagatcagacagctgcccaatctgctgcggggct |
| | B/Austria/ | atgaacatgtgcggctgagcacccacaacgtgatcaacacagaggatgcccctggcggcccttacgagatcgg |
| | 1359417/2021 | aacatctggctcttgcctgaacattaccaacggcaagggcttcttcgccaccatggcttgggccgtgcctaag |
| | | aacaagaccgccaccaatccactgaccatcgaggtgccctacatctgtaccgaggaagaagatcagatcaccg |
| | | tctggggcttccacagcgacgacgaaacacagatggccagactgtacggcgacagcaagcctcagaagttcac |
| | | cagctctgccaacggcgtgaccacacactacgtgtcccagatcggcggcttccccaatcagacagaagatggc |
| | | ggactgccccagagcggaagaatcgtggtggactacatggtgcagaagtccggcaagaccggcacaatcacat |
| | | accagcggggaatcctgctgcctcagaaagtttggtgcgccagcggcaagagcaaagtgatcaagggcagcct |
| | | gcctctgatcggcgaagccgattgtctgcacgagaagtacggcggcctgaacaagagcaagccctactacaca |
| | | ggcgagcacgccaaggccatcggcaactgtcctatctgggtcaagacccctctgaagctggccaacggcacca |
| | | agtatagacctccagccaagctgctgaaagagcggggcttctttggagctatcgccggctttcttgaaggcgg |
| | | ctgggagggaatgattgccggctggcatggctacacatctcatggcgcacatggcgtggcagtggccgctgat |
| | | ctgaagtctacacaagaggccatcaacaagatcaccaagaacctgaacagcctgagcgagctggaagtgaaga |
| | | acctgcagagactgtccggcgccatggacgagctgcacaacgagatcctggaactggacgagaaggtggacga |
| | | cctgagagccgataccatctccagccagattgagctggcagtgctgctgtccaacgagggcatcatcaacagc |
| | | gaggacgagcatctgctggccctggaacggaagctgaagaagatgctgggcccaagcgccgtggaaatcggca |
| | | atggctgcttcgagacaaagcacaagtgcaaccagacctgcctggacagaattgccgccggaacatttgacgc |
| | | cggcgagtttagcctgcctaccttcgactccctgaacatcacagccgccagcctgaatgacgacggcctggac |
| | | aatcacaccatcctgctgtactactccaccgccgcttctagcctggccgtgacactgatgatcgccatctttg |
| | | tggtgtacatggtgtccagagacaacgtgtcctgcagcatctgcctgtgatgactcgagctggtactgcatgc |
| | | acgcaatgctagctgcccctttcccgtcctgggtaccccgagtctcccccgacctcgggtcccaggtatgctc |
| | | ccacctccacctgccccactcaccacctctgctagttccagacacctcccaagcacgcagcaatgcagctcaa |
| | | aacgcttagcctagccacacccccacgggaaacagcagtgattaacctttagcaataaacgaaagtttaacta |
| | | agctatactaaccccagggttggtcaatttcgtgccagccacaccctggagctagcaaaaaaaaaaaaaaaaa |
| | | aaaaaaaaaaaaagcatatgactaaaaaaaaaaaaaaaaaaaaaaaaaaaaaaaaaaaaaaaaaaaaaaaaaa |
| | | aaaaaaaaaaaaaaaaaaa |
| 89 | Full length | agaauaaacuaguauucuucugguccccacagacucagagagaacccgccaccaugaaggccaucaucgugcu |
| | RNA construct | gcugaugguggucaccagcaacgccgacagaaucugcaccggcaucaccagcagcaacagcccucacgugguc |
| | sequence | aagacagccacacagggcgaagugaaugugaccggcgugaucccucugaccaccacaccuaccaagagccacu |
| | encoding | ucgccaaccugaagggcaccgagacaagaggcaagcugugccccaagugccugaacugcaccgaucuggaugu |
| | an HA | ggcccugggcagaccuaaguguaccggcaagaucccuagcgccagaguguccauccugcacgaagugcggccu |
| | protein of | gugaccagcggcugcuuucccauuaugcacgaccggaccaagaucagacagcugcccaaucugcugcggggcu |
| | B/Austria/ | augaacaugugcggcugagcacccacaacgugaucaacacagaggaugccccuggcggcccuuacgagaucgg |
| | 1359417/2021 | aacaucuggcucuugccugaacauuaccaacggcaagggcuucuucgccaccauggcuugggccgugccuaag |
| | | aacaagaccgccaccaauccacugaccaucgaggugcccuacaucuguaccgaggaagaagaucagaucaccg |
| | | ucuggggcuuccacagcgacgacgaaacacagauggccagacuguacggcgacagcaagccucagaaguucac |
| | | cagcucugccaacggcgugaccacacacuacgugucccagaucggcggcuuccccaaucagacagaagauggc |
| | | ggacugccccagagcggaagaaucgugguggacuacauggugcagaaguccggcaagaccggcacaaucacau |
| | | accagcggggaauccugcugccucagaaaguuuggugcgccagcggcaagagcaaagugaucaagggcagccu |
| | | gccucugaucggcgaagccgauugucugcacgagaaguacggcggccugaacaagagcaagcccuacuacaca |
| | | ggcgagcacgccaaggccaucggcaacuguccuaucugggucaagaccccucugaagcuggccaacggcacca |
| | | aguauagaccuccagccaagcugcugaaagagcggggcuucuuuggagcuaucgccggcuuucuugaaggcgg |
| | | cugggagggaaugauugccggcuggcauggcuacacaucucauggcgcacauggcguggcaguggccgcugau |
| | | cugaagucuacacaagaggccaucaacaagaucaccaagaaccugaacagccugagcgagcuggaagugaaga |
| | | accugcagagacuguccggcgccauggacgagcugcacaacgagauccuggaacuggacgagaagguggacga |
| | | ccugagagccgauaccaucuccagccagauugagcuggcagugcugcuguccaacgagggcaucaucaacagc |
| | | gaggacgagcaucugcuggcccuggaacggaagcugaagaagaugcugggcccaagcgccguggaaaucggca |
| | | auggcugcuucgagacaaagcacaagugcaaccagaccugccuggacagaauugccgccggaacauuugacgc |
| | | cggcgaguuuagccugccuaccuucgacucccugaacaucacagccgccagccugaaugacgacggccuggac |
| | | aaucacaccauccugcuguacuacuccaccgccgcuucuagccuggccgugacacugaugaucgccaucuuug |
| | | ugguguacaugguguccagagacaacguguccugcagcaucugccugugaugacucgagcugguacugcaugc |
| | | acgcaaugcuagcugccccuuucccguccuggguaccccgagucucccccgaccucgggucccagguaugcuc |
| | | ccaccuccaccugccccacucaccaccucugcuaguuccagacaccucccaagcacgcagcaaugcagcucaa |
| | | aacgcuuagccuagccacacccccacgggaaacagcagugauuaaccuuuagcaauaaacgaaaguuuaacua |
| | | agcuauacuaaccccaggguuggucaauuucgugccagccacacccuggagcuagcaaaaaaaaaaaaaaaaa |
| | | aaaaaaaaaaaaagcauaugacuaaaaaaaaaaaaaaaaaaaaaaaaaaaaaaaaaaaaaaaaaaaaaaaaaa |
| | | aaaaaaaaaaaaaaaaaaa |

TABLE 19
Sequence of one embodiment of a A/Wisconsin/588/2019-specific Influenza RNA vaccine

| SEQ | | |
|---|---|---|
| ID | Brief | |
| NO. | Description | Sequence |
| 90 | Amino acid | MKAILVVMLYTFTTANADTLCIGYHANNSTDTVDTVLEKNVTVTHSVNLLEDKHNGKLCKLRGVAPLHLGKCNI |
| | sequence of | AGWILGNPECESLSTARSWSYIVETSNSDNGTCYPGDFINYEELREQLSSVSSFERFEIFPKTSSWPNHDSDNG |
| | RNA-encoded | VTAACPHAGAKSFYKNLIWLVKKGKSYPKINQTYINDKGKEVLVLWGIHHPPTIADQQSLYQNADAYVFVGTSR |
| | Influenza HA | YSKKFKPEIATRPKVRDQEGRMNYYWTLVEPGDKITFEATGNLVAPRYAFTMERDAGSGIIISDTPVHDCNTTC |
| | protein of | QTPEGAINTSLPFQNVHPITIGKCPKYVKSTKLRLATGLRNVPSIQSRGLFGAIAGFIEGGWTGMVDGWYGYHH |
| | A/Wisconsin/ | QNEQGSGYAADLKSTQNAIDKITNKVNSVIEKMNTQFTAVGKEFNHLEKRIENLNKKVDDGFLDIWTYNAELLV |
| | 588/2019 | LLENERTLDYHDSNVKNLYEKVRNQLKNNAKEIGNGCFEFYHKCDNTCMESVKNGTYDYPKYSEEAKLNREKID |
| | | GVKLDSTRIYQILAIYSTVASSLVLVVSLGAISFWMCSNGSLQCRICI |
| 91 | DNA sequence | atgaaggccatcctggtggtcatgctgtacaccttcaccaccgccaacgccgacacactgtgtatcggctacca |
| | encoding an | cgccaacaacagcaccgacaccgtggataccgtgctggaaaagaacgtgaccgtgacacacagcgtgaacctgc |
| | influenza HA | tggaagataagcacaacggcaagctgtgcaagctgagaggcgtggcacctctgcacctgggcaagtgtaatatc |
| | protein of | gccggctggatcctgggcaaccctgagtgtgaaagcctgagcaccgccagatcctggtcctacatcgtggaaac |
| | A/Wisconsin/ | cagcaacagcgacaacggcacatgctaccccggcgacttcatcaactacgaggaactgcgggaacagctgagca |
| | 588/2019 | gcgtgtccagcttcgagagattcgagatcttccccaagaccagcagctggcccaaccacgactctgacaatggc |
| | | gtgacagccgcctgtcctcatgccggcgctaagagcttctacaagaacctgatctggctggtcaagaagggcaa |
| | | gagctaccccaagatcaaccagacctacatcaacgacaagggcaaagaggtgctggtcctctggggcatccacc |
| | | atcctccaacaatcgccgatcagcagagcctgtaccagaacgccgatgcctatgtgttcgtgggcaccagccgg |
| | | tacagcaagaagttcaagcccgagatcgccaccaggcctaaagtgcgggatcaagagggcagaatgaactacta |
| | | ctggaccctggtggaacccggcgacaagatcacatttgaggccaccggcaatctggtggcccctagatacgcct |
| | | tcaccatggaaagagatgccggcagcggcatcatcatcagcgatacccctgtgcacgactgcaacaccacctgt |
| | | cagacacctgagggcgccatcaataccagcctgcctttccagaacgtgcaccccatcaccatcggcaagtgccc |
| | | caaatacgtgaagtccaccaagctgaggctggccacaggcctgagaaatgtgccctccatccagagcagaggcc |
| | | tgtttggagccattgccggctttatcgaaggcggctggacaggcatggtggacggatggtacggataccaccac |
| | | cagaacgagcaaggctctggctatgccgccgacctgaagtctacccagaatgccatcgataagatcaccaacaa |
| | | agtgaacagcgtgatcgagaagatgaacacccagttcaccgccgtgggaaaagagttcaaccacctggaaaagc |
| | | gcatcgagaacctgaacaagaaggtggacgacggcttcctggacatctggacctacaatgccgaactgctggtg |
| | | ctgctggagaacgagagaaccctggactaccacgacagcaacgtgaagaacctgtacgagaaagtgcgcaacca |
| | | gctgaagaacaacgccaaagagatcggcaacggctgcttcgagttctaccacaagtgcgacaatacctgcatgg |
| | | aaagcgtgaagaatggcacctacgactaccctaagtacagcgaggaagccaagctgaaccgcgagaagatcgac |
| | | ggcgtgaagctggatagcacccggatctaccagattctggccatctacagcaccgtggcctctagcctggtgct |
| | | ggtggtttctctgggcgctatcagcttctggatgtgcagcaatggcagcctgcagtgccggatctgcatctgat |
| | | ga |
| 92 | RNA sequence | augaaggccauccugguggucaugcuguacaccuucaccaccgccaacgccgacacacuguguaucggcuacca |
| | encoding an | cgccaacaacagcaccgacaccguggauaccgugcuggaaaagaacgugaccgugacacacagcgugaaccugc |
| | influenza HA | uggaagauaagcacaacggcaagcugugcaagcugagaggcguggcaccucugcaccugggcaaguguaauauc |
| | protein of | gccggcuggauccugggcaacccugagugugaaagccugagcaccgccagauccugguccuacaucguggaaac |
| | A/Wisconsin/ | cagcaacagcgacaacggcacaugcuaccccggcgacuucaucaacuacgaggaacugcgggaacagcugagca |
| | 588/2019 | gcguguccagcuucgagagauucgagaucuuccccaagaccagcagcuggcccaaccacgacucugacaauggc |
| | | gugacagccgccuguccucaugccggcgcuaagagcuucuacaagaaccugaucuggcuggucaagaagggcaa |
| | | gagcuaccccaagaucaaccagaccuacaucaacgacaagggcaaagaggugcugguccucuggggcauccacc |
| | | auccuccaacaaucgccgaucagcagagccuguaccagaacgccgaugccuauguguucgugggcaccagccgg |
| | | uacagcaagaaguucaagcccgagaucgccaccaggccuaaagugcgggaucaagagggcagaaugaacuacua |
| | | cuggacccugguggaacccggcgacaagaucacauuugaggccaccggcaaucugguggccccuagauacgccu |
| | | ucaccauggaaagagaugccggcagcggcaucaucaucagcgauaccccugugcacgacugcaacaccaccugu |
| | | cagacaccugagggcgccaucaauaccagccugccuuuccagaacgugcaccccaucaccaucggcaagugccc |
| | | caaauacgugaaguccaccaagcugaggcuggccacaggccugagaaaugugcccuccauccagagcagaggcc |
| | | uguuuggagccauugccggcuuuaucgaaggcggcuggacaggcaugguggacggaugguacggauaccaccac |
| | | cagaacgagcaaggcucuggcuaugccgccgaccugaagucuacccagaaugccaucgauaagaucaccaacaa |
| | | agugaacagcgugaucgagaagaugaacacccaguucaccgccgugggaaaagaguucaaccaccuggaaaagc |
| | | gcaucgagaaccugaacaagaagguggacgacggcuuccuggacaucuggaccuacaaugccgaacugcuggug |
| | | cugcuggagaacgagagaacccuggacuaccacgacagcaacgugaagaaccuguacgagaaagugcgcaacca |
| | | gcugaagaacaacgccaaagagaucggcaacggcugcuucgaguucuaccacaagugcgacaauaccugcaugg |
| | | aaagcgugaagaauggcaccuacgacuacccuaaguacagcgaggaagccaagcugaaccgcgagaagaucgac |
| | | ggcgugaagcuggauagcacccggaucuaccagauucuggccaucuacagcaccguggccucuagccuggugcu |
| | | ggugguuucucugggcgcuaucagcuucuggaugugcagcaauggcagccugcagugccggaucugcaucugau |
| | | ga |
| 93 | Full length | agaataaactagtattcttctggtccccacagactcagagagaacccgccaccatgaaggccatcctggtggtc |
| | DNA construct | atgctgtacaccttcaccaccgccaacgccgacacactgtgtatcggctaccacgccaacaacagcaccgacac |
| | sequence | cgtggataccgtgctggaaaagaacgtgaccgtgacacacagcgtgaacctgctggaagataagcacaacggca |
| | encoding | agctgtgcaagctgagaggcgtggcacctctgcacctgggcaagtgtaatatcgccggctggatcctgggcaac |
| | an HA | cctgagtgtgaaagcctgagcaccgccagatcctggtcctacatcgtggaaaccagcaacagcgacaacggcac |
| | protein of | atgctaccccggcgacttcatcaactacgaggaactgcgggaacagctgagcagcgtgtccagcttcgagagat |
| | A/Wisconsin/ | tcgagatcttccccaagaccagcagctggcccaaccacgactctgacaatggcgtgacagccgcctgtcctcat |
| | 588/2019 | gccggcgctaagagcttctacaagaacctgatctggctggtcaagaagggcaagagctaccccaagatcaacca |
| | | gacctacatcaacgacaagggcaaagaggtgctggtcctctggggcatccaccatcctccaacaatcgccgatc |
| | | agcagagcctgtaccagaacgccgatgcctatgtgttcgtgggcaccagccggtacagcaagaagttcaagccc |
| | | gagatcgccaccaggcctaaagtgcgggatcaagagggcagaatgaactactactggaccctggtggaacccgg |
| | | cgacaagatcacatttgaggccaccggcaatctggtggcccctagatacgccttcaccatggaaagagatgccg |
| | | gcagcggcatcatcatcagcgatacccctgtgcacgactgcaacaccacctgtcagacacctgagggcgccatc |
| | | aataccagcctgcctttccagaacgtgcaccccatcaccatcggcaagtgccccaaatacgtgaagtccaccaa |
| | | gctgaggctggccacaggcctgagaaatgtgccctccatccagagcagaggcctgtttggagccattgccggct |
| | | ttatcgaaggcggctggacaggcatggtggacggatggtacggataccaccaccagaacgagcaaggctctggc |
| | | tatgccgccgacctgaagtctacccagaatgccatcgataagatcaccaacaaagtgaacagcgtgatcgagaa |
| | | gatgaacacccagttcaccgccgtgggaaaagagttcaaccacctggaaaagcgcatcgagaacctgaacaaga |
| | | aggtggacgacggcttcctggacatctggacctacaatgccgaactgctggtgctgctggagaacgagagaacc |
| | | ctggactaccacgacagcaacgtgaagaacctgtacgagaaagtgcgcaaccagctgaagaacaacgccaaaga |
| | | gatcggcaacggctgcttcgagttctaccacaagtgcgacaatacctgcatggaaagcgtgaagaatggcacct |
| | | acgactaccctaagtacagcgaggaagccaagctgaaccgcgagaagatcgacggcgtgaagctggatagcacc |
| | | cggatctaccagattctggccatctacagcaccgtggcctctagcctggtgctggtggtttctctgggcgctat |
| | | cagcttctggatgtgcagcaatggcagcctgcagtgccggatctgcatctgatgactcgagctggtactgcatg |
| | | cacgcaatgctagctgcccctttcccgtcctgggtaccccgagtctcccccgacctcgggtcccaggtatgctc |
| | | ccacctccacctgccccactcaccacctctgctagttccagacacctcccaagcacgcagcaatgcagctcaaa |
| | | acgcttagcctagccacacccccacgggaaacagcagtgattaacctttagcaataaacgaaagtttaactaag |
| | | ctatactaaccccagggttggtcaatttcgtgccagccacaccctggagctagcaaaaaaaaaaaaaaaaaaaa |
| | | aaaaaaaaaagcatatgactaaaaaaaaaaaaaaaaaaaaaaaaaaaaaaaaaaaaaaaaaaaaaaaaaaaaaa |
| | | aaaaaaaaaaaaaaaa |
| 94 | Full length | agaauaaacuaguauucuucugguccccacagacucagagagaacccgccaccaugaaggccauccuggugguc |
| | RNA construct | augcuguacaccuucaccaccgccaacgccgacacacuguguaucggcuaccacgccaacaacagcaccgacac |
| | sequence | cguggauaccgugcuggaaaagaacgugaccgugacacacagcgugaaccugcuggaagauaagcacaacggca |
| | encoding | agcugugcaagcugagaggcguggcaccucugcaccugggcaaguguaauaucgccggcuggauccugggcaac |
| | an HA | ccugagugugaaagccugagcaccgccagauccugguccuacaucguggaaaccagcaacagcgacaacggcac |
| | protein of | augcuaccccggcgacuucaucaacuacgaggaacugcgggaacagcugagcagcguguccagcuucgagagau |
| | A/Wisconsin/ | ucgagaucuuccccaagaccagcagcuggcccaaccacgacucugacaauggcgugacagccgccuguccucau |
| | 588/2019 | gccggcgcuaagagcuucuacaagaaccugaucuggcuggucaagaagggcaagagcuaccccaagaucaacca |
| | | gaccuacaucaacgacaagggcaaagaggugcugguccucuggggcauccaccauccuccaacaaucgccgauc |
| | | agcagagccuguaccagaacgccgaugccuauguguucgugggcaccagccgguacagcaagaaguucaagccc |
| | | gagaucgccaccaggccuaaagugcgggaucaagagggcagaaugaacuacuacuggacccugguggaacccgg |
| | | cgacaagaucacauuugaggccaccggcaaucugguggccccuagauacgccuucaccauggaaagagaugccg |
| | | gcagcggcaucaucaucagcgauaccccugugcacgacugcaacaccaccugucagacaccugagggcgccauc |
| | | aauaccagccugccuuuccagaacgugcaccccaucaccaucggcaagugccccaaauacgugaaguccaccaa |
| | | gcugaggcuggccacaggccugagaaaugugcccuccauccagagcagaggccuguuuggagccauugccggcu |
| | | uuaucgaaggcggcuggacaggcaugguggacggaugguacggauaccaccaccagaacgagcaaggcucuggc |
| | | uaugccgccgaccugaagucuacccagaaugccaucgauaagaucaccaacaaagugaacagcgugaucgagaa |
| | | gaugaacacccaguucaccgccgugggaaaagaguucaaccaccuggaaaagcgcaucgagaaccugaacaaga |
| | | agguggacgacggcuuccuggacaucuggaccuacaaugccgaacugcuggugcugcuggagaacgagagaacc |
| | | cuggacuaccacgacagcaacgugaagaaccuguacgagaaagugcgcaaccagcugaagaacaacgccaaaga |
| | | gaucggcaacggcugcuucgaguucuaccacaagugcgacaauaccugcauggaaagcgugaagaauggcaccu |
| | | acgacuacccuaaguacagcgaggaagccaagcugaaccgcgagaagaucgacggcgugaagcuggauagcacc |
| | | cggaucuaccagauucuggccaucuacagcaccguggccucuagccuggugcuggugguuucucugggcgcuau |
| | | cagcuucuggaugugcagcaauggcagccugcagugccggaucugcaucugaugacucgagcugguacugcaug |
| | | cacgcaaugcuagcugccccuuucccguccuggguaccccgagucucccccgaccucgggucccagguaugcuc |
| | | ccaccuccaccugccccacucaccaccucugcuaguuccagacaccucccaagcacgcagcaaugcagcucaaa |
| | | acgcuuagccuagccacacccccacgggaaacagcagugauuaaccuuuagcaauaaacgaaaguuuaacuaag |
| | | cuauacuaaccccaggguuggucaauuucgugccagccacacccuggagcuagcaaaaaaaaaaaaaaaaaaaa |
| | | aaaaaaaaaagcauaugacuaaaaaaaaaaaaaaaaaaaaaaaaaaaaaaaaaaaaaaaaaaaaaaaaaaaaaa |
| | | aaaaaaaaaaaaaaaa |

TABLE 20
Sequence of one embodiment of a A/Cambodia/e0826360/2020-specific Influenza RNA vaccine

| SEQ | | |
|---|---|---|
| ID | Brief | |
| NO. | Description | Sequence |
| 95 | Amino acid | MKTIIALSYILCLVFAQKIPGNDNSTATLCLGHHAVPNGTIVKTITNDRIEVTNATELVQNSSIGEICDSPHQI |
| | sequence of | LDGGNCTLIDALLGDPQCDGFQNKEWDLFVERSRANSNCYPYDVPDYASLRSLVASSGTLEFKNESFNWTGVKQ |
| | RNA-encoded | NGTSSACIRGSSSSFFSRLNWLTHLNYTYPALNVTMPNNEQFDKLYIWGVHHPSTDKDQISLFAQPSGRITVST |
| | Influenza HA | KRSQQAVIPNIGSRPRIRDIPSRISIYWTIVKPGDILLINSTGNLIAPRGYFKIRSGKSSIMRSDAPIGKCKSE |
| | protein of | CITPNGSIPNDKPFQNVNRITYGACPRYVKQSTLKLATGMRNVPEKQTRGIFGAIAGFIENGWEGMVDGWYGFR |
| | A/Cambodia/ | HQNSEGRGQAADLKSTQAAIDQINGKLNRLIGKTNEKFHQIEKEFSEVEGRVQDLEKYVEDTKIDLWSYNAELL |
| | e0826360/2020 | VALENQHTIDLTDSEMNKLFEKTKKQLRENAEDMGNGCFKIYHKCDNACIGSIRNETYDHNVYRDEALNNRFQI |
| | | KGVELKSGYKDWILWISFAMSCFLLCIALLGFIMWACQKGNIRCNICI |
| 96 | DNA sequence | atgaagaccatcattgccctgagctacatcctgtgcctggtgttcgcccagaagatccccggcaacgacaatag |
| | encoding an | caccgccacactgtgtctgggccatcacgctgtgcctaacggcaccatcgtgaaaaccatcaccaacgaccgga |
| | influenza HA | tcgaagtgaccaacgccacagagctggtgcagaacagcagcatcggcgagatctgcgatagccctcaccagatc |
| | protein of | ctggacggcggcaattgcacactgatcgatgccctgctgggcgaccctcagtgtgatggcttccagaacaaaga |
| | A/Cambodia/ | atgggacctgttcgtggaaagaagccgggccaacagcaactgctacccctacgatgtgcccgactacgccagcc |
| | e0826360/2020 | tgagatctctggtggcctctagcggcaccctggaattcaagaacgagagcttcaactggaccggcgtgaagcag |
| | | aatggcaccagcagcgcctgtatcagaggcagcagctccagcttcttcagcagactgaactggctgacccacct |
| | | gaactacacataccccgctctgaacgtgaccatgcctaacaacgagcagttcgacaagctgtacatctggggcg |
| | | tgcaccatcctagcaccgacaaggatcagatcagcctgtttgcccagcctagcggcagaatcaccgtgtccact |
| | | aagagaagccagcaggccgtgattcccaacatcggcagcagaccccggatcagagacatccccagccggatcag |
| | | catctactggacaatcgtgaagcccggcgacatcctgctgatcaacagcaccggcaatctgatcgcccctcggg |
| | | gctacttcaagatcagaagcggcaagagcagcatcatgcggagcgacgcccctatcggcaagtgcaagagcgag |
| | | tgcatcaccccaaacggcagcatccccaacgacaagcccttccagaatgtgaaccggatcacctacggcgcctg |
| | | tcctagatacgtgaaacagagcaccctgaagctggccaccggcatgagaaacgtgccagagaagcagaccagag |
| | | gcatcttcggagccattgccggcttcatcgagaacggctgggaaggcatggtggacggatggtacggcttcaga |
| | | caccagaacagcgaaggcagaggacaggccgctgacctgaaatctacacaggccgccatcgaccagatcaacgg |
| | | caagctgaaccggctgatcggcaagaccaacgagaagttccaccagatcgagaaagagttcagcgaggtcgagg |
| | | gcagagtgcaggacctcgaaaaatacgtggaagataccaagatcgacctgtggtcctacaatgccgaactgctg |
| | | gtggccctggaaaaccagcacaccatcgacctgaccgacagcgagatgaacaagctgttcgaaaagaccaagaa |
| | | gcagctgcgcgagaacgccgaggatatgggcaacggctgctttaagatctaccacaagtgcgacaacgcctgca |
| | | tcggctccatccggaacgagacatacgaccacaacgtgtacagagatgaggccctgaacaaccggttccagatc |
| | | aaaggcgtggaactgaagtccggctacaaggactggatactgtggatcagcttcgccatgagctgctttctgct |
| | | gtgtatcgctctgctgggcttcatcatgtgggcctgccagaaaggcaacatccggtgcaacatctgcatctgat |
| | | ga |
| 97 | RNA sequence | augaagaccaucauugcccugagcuacauccugugccugguguucgcccagaagauccccggcaacgacaauag |
| | encoding an | caccgccacacugugucugggccaucacgcugugccuaacggcaccaucgugaaaaccaucaccaacgaccgga |
| | influenza HA | ucgaagugaccaacgccacagagcuggugcagaacagcagcaucggcgagaucugcgauagcccucaccagauc |
| | protein of | cuggacggcggcaauugcacacugaucgaugcccugcugggcgacccucagugugauggcuuccagaacaaaga |
| | A/Cambodia/ | augggaccuguucguggaaagaagccgggccaacagcaacugcuaccccuacgaugugcccgacuacgccagcc |
| | e0826360/2020 | ugagaucucugguggccucuagcggcacccuggaauucaagaacgagagcuucaacuggaccggcgugaagcag |
| | | aauggcaccagcagcgccuguaucagaggcagcagcuccagcuucuucagcagacugaacuggcugacccaccu |
| | | gaacuacacauaccccgcucugaacgugaccaugccuaacaacgagcaguucgacaagcuguacaucuggggcg |
| | | ugcaccauccuagcaccgacaaggaucagaucagccuguuugcccagccuagcggcagaaucaccguguccacu |
| | | aagagaagccagcaggccgugauucccaacaucggcagcagaccccggaucagagacauccccagccggaucag |
| | | caucuacuggacaaucgugaagcccggcgacauccugcugaucaacagcaccggcaaucugaucgccccucggg |
| | | gcuacuucaagaucagaagcggcaagagcagcaucaugcggagcgacgccccuaucggcaagugcaagagcgag |
| | | ugcaucaccccaaacggcagcauccccaacgacaagcccuuccagaaugugaaccggaucaccuacggcgccug |
| | | uccuagauacgugaaacagagcacccugaagcuggccaccggcaugagaaacgugccagagaagcagaccagag |
| | | gcaucuucggagccauugccggcuucaucgagaacggcugggaaggcaugguggacggaugguacggcuucaga |
| | | caccagaacagcgaaggcagaggacaggccgcugaccugaaaucuacacaggccgccaucgaccagaucaacgg |
| | | caagcugaaccggcugaucggcaagaccaacgagaaguuccaccagaucgagaaagaguucagcgaggucgagg |
| | | gcagagugcaggaccucgaaaaauacguggaagauaccaagaucgaccugugguccuacaaugccgaacugcug |
| | | guggcccuggaaaaccagcacaccaucgaccugaccgacagcgagaugaacaagcuguucgaaaagaccaagaa |
| | | gcagcugcgcgagaacgccgaggauaugggcaacggcugcuuuaagaucuaccacaagugcgacaacgccugca |
| | | ucggcuccauccggaacgagacauacgaccacaacguguacagagaugaggcccugaacaaccgguuccagauc |
| | | aaaggcguggaacugaaguccggcuacaaggacuggauacuguggaucagcuucgccaugagcugcuuucugcu |
| | | guguaucgcucugcugggcuucaucaugugggccugccagaaaggcaacauccggugcaacaucugcaucugau |
| | | ga |
| 98 | Full length | agaataaactagtattcttctggtccccacagactcagagagaacccgccaccatgaagaccatcattgccctg |
| | DNA construct | agctacatcctgtgcctggtgttcgcccagaagatccccggcaacgacaatagcaccgccacactgtgtctggg |
| | sequence | ccatcacgctgtgcctaacggcaccatcgtgaaaaccatcaccaacgaccggatcgaagtgaccaacgccacag |
| | encoding | agctggtgcagaacagcagcatcggcgagatctgcgatagccctcaccagatcctggacggcggcaattgcaca |
| | an HA | ctgatcgatgccctgctgggcgaccctcagtgtgatggcttccagaacaaagaatgggacctgttcgtggaaag |
| | protein of | aagccgggccaacagcaactgctacccctacgatgtgcccgactacgccagcctgagatctctggtggcctcta |
| | A/Cambodia/ | gcggcaccctggaattcaagaacgagagcttcaactggaccggcgtgaagcagaatggcaccagcagcgcctgt |
| | e0826360/2020 | atcagaggcagcagctccagcttcttcagcagactgaactggctgacccacctgaactacacataccccgctct |
| | | gaacgtgaccatgcctaacaacgagcagttcgacaagctgtacatctggggcgtgcaccatcctagcaccgaca |
| | | aggatcagatcagcctgtttgcccagcctagcggcagaatcaccgtgtccactaagagaagccagcaggccgtg |
| | | attcccaacatcggcagcagaccccggatcagagacatccccagccggatcagcatctactggacaatcgtgaa |
| | | gcccggcgacatcctgctgatcaacagcaccggcaatctgatcgcccctcggggctacttcaagatcagaagcg |
| | | gcaagagcagcatcatgcggagcgacgcccctatcggcaagtgcaagagcgagtgcatcaccccaaacggcagc |
| | | atccccaacgacaagcccttccagaatgtgaaccggatcacctacggcgcctgtcctagatacgtgaaacagag |
| | | caccctgaagctggccaccggcatgagaaacgtgccagagaagcagaccagaggcatcttcggagccattgccg |
| | | gcttcatcgagaacggctgggaaggcatggtggacggatggtacggcttcagacaccagaacagcgaaggcaga |
| | | ggacaggccgctgacctgaaatctacacaggccgccatcgaccagatcaacggcaagctgaaccggctgatcgg |
| | | caagaccaacgagaagttccaccagatcgagaaagagttcagcgaggtcgagggcagagtgcaggacctcgaaa |
| | | aatacgtggaagataccaagatcgacctgtggtcctacaatgccgaactgctggtggccctggaaaaccagcac |
| | | accatcgacctgaccgacagcgagatgaacaagctgttcgaaaagaccaagaagcagctgcgcgagaacgccga |
| | | ggatatgggcaacggctgctttaagatctaccacaagtgcgacaacgcctgcatcggctccatccggaacgaga |
| | | catacgaccacaacgtgtacagagatgaggccctgaacaaccggttccagatcaaaggcgtggaactgaagtcc |
| | | ggctacaaggactggatactgtggatcagcttcgccatgagctgctttctgctgtgtatcgctctgctgggctt |
| | | catcatgtgggcctgccagaaaggcaacatccggtgcaacatctgcatctgatgactcgagctggtactgcatg |
| | | cacgcaatgctagctgcccctttcccgtcctgggtaccccgagtctcccccgacctcgggtcccaggtatgctc |
| | | ccacctccacctgccccactcaccacctctgctagttccagacacctcccaagcacgcagcaatgcagctcaaa |
| | | acgcttagcctagccacacccccacgggaaacagcagtgattaacctttagcaataaacgaaagtttaactaag |
| | | ctatactaaccccagggttggtcaatttcgtgccagccacaccctggagctagcaaaaaaaaaaaaaaaaaaaa |
| | | aaaaaaaaaagcatatgactaaaaaaaaaaaaaaaaaaaaaaaaaaaaaaaaaaaaaaaaaaaaaaaaaaaaaa |
| | | aaaaaaaaaaaaaaaa |
| 99 | Full length | agaauaaacuaguauucuucugguccccacagacucagagagaacccgccaccaugaagaccaucauugcccug |
| | RNA construct | agcuacauccugugccugguguucgcccagaagauccccggcaacgacaauagcaccgccacacugugucuggg |
| | sequence | ccaucacgcugugccuaacggcaccaucgugaaaaccaucaccaacgaccggaucgaagugaccaacgccacag |
| | encoding | agcuggugcagaacagcagcaucggcgagaucugcgauagcccucaccagauccuggacggcggcaauugcaca |
| | an HA | cugaucgaugcccugcugggcgacccucagugugauggcuuccagaacaaagaaugggaccuguucguggaaag |
| | protein of | aagccgggccaacagcaacugcuaccccuacgaugugcccgacuacgccagccugagaucucugguggccucua |
| | A/Cambodia/ | gcggcacccuggaauucaagaacgagagcuucaacuggaccggcgugaagcagaauggcaccagcagcgccugu |
| | e0826360/2020 | aucagaggcagcagcuccagcuucuucagcagacugaacuggcugacccaccugaacuacacauaccccgcucu |
| | | gaacgugaccaugccuaacaacgagcaguucgacaagcuguacaucuggggcgugcaccauccuagcaccgaca |
| | | aggaucagaucagccuguuugcccagccuagcggcagaaucaccguguccacuaagagaagccagcaggccgug |
| | | auucccaacaucggcagcagaccccggaucagagacauccccagccggaucagcaucuacuggacaaucgugaa |
| | | gcccggcgacauccugcugaucaacagcaccggcaaucugaucgccccucggggcuacuucaagaucagaagcg |
| | | gcaagagcagcaucaugcggagcgacgccccuaucggcaagugcaagagcgagugcaucaccccaaacggcagc |
| | | auccccaacgacaagcccuuccagaaugugaaccggaucaccuacggcgccuguccuagauacgugaaacagag |
| | | cacccugaagcuggccaccggcaugagaaacgugccagagaagcagaccagaggcaucuucggagccauugccg |
| | | gcuucaucgagaacggcugggaaggcaugguggacggaugguacggcuucagacaccagaacagcgaaggcaga |
| | | ggacaggccgcugaccugaaaucuacacaggccgccaucgaccagaucaacggcaagcugaaccggcugaucgg |
| | | caagaccaacgagaaguuccaccagaucgagaaagaguucagcgaggucgagggcagagugcaggaccucgaaa |
| | | aauacguggaagauaccaagaucgaccugugguccuacaaugccgaacugcugguggcccuggaaaaccagcac |
| | | accaucgaccugaccgacagcgagaugaacaagcuguucgaaaagaccaagaagcagcugcgcgagaacgccga |
| | | ggauaugggcaacggcugcuuuaagaucuaccacaagugcgacaacgccugcaucggcuccauccggaacgaga |
| | | cauacgaccacaacguguacagagaugaggcccugaacaaccgguuccagaucaaaggcguggaacugaagucc |
| | | ggcuacaaggacuggauacuguggaucagcuucgccaugagcugcuuucugcuguguaucgcucugcugggcuu |
| | | caucaugugggccugccagaaaggcaacauccggugcaacaucugcaucugaugacucgagcugguacugcaug |
| | | cacgcaaugcuagcugccccuuucccguccuggguaccccgagucucccccgaccucgggucccagguaugcuc |
| | | ccaccuccaccugccccacucaccaccucugcuaguuccagacaccucccaagcacgcagcaaugcagcucaaa |
| | | acgcuuagccuagccacacccccacgggaaacagcagugauuaaccuuuagcaauaaacgaaaguuuaacuaag |
| | | cuauacuaaccccaggguuggucaauuucgugccagccacacccuggagcuagcaaaaaaaaaaaaaaaaaaaa |
| | | aaaaaaaaaagcauaugacuaaaaaaaaaaaaaaaaaaaaaaaaaaaaaaaaaaaaaaaaaaaaaaaaaaaaaa |
| | | aaaaaaaaaaaaaaaa |

TABLE 21
Sequence of one embodiment of a B/Washington/02/2019-specific Influenza RNA vaccine

| SEQ | | |
|---|---|---|
| ID | Brief | |
| NO. | Description | Sequence |
| 100 | Amino acid | MKAIIVLLMVVTSNADRICTGITSSNSPHVVKTATQGEVNVTGVIPLTTTPTKSHFANLKGTETRGKLCPKCL |
| | sequence of | NCTDLDVALGRPKCTGKIPSARVSILHEVRPVTSGCFPIMHDRTKIRQLPNLLRGYEHVRLSTHNVINAEDAP |
| | RNA-encoded | GRPYEIGTSGSCPNITNGNGFFATMAWAVPKNKTATNPLTIEVPYICTEGEDQITVWGFHSDNETQMAKLYGD |
| | Influenza HA | SKPQKFTSSANGVTTHYVSQIGGFPNQTEDGGLPQSGRIVVDYMVQKSGKTGTITYQRGILLPQKVWCASGRS |
| | protein of | KVIKGSLPLIGEADCLHEKYGGLNKSKPYYTGEHAKAIGNCPIWVKTPLKLANGTKYRPPAKLLKERGFFGAI |
| | B/Washington/ | AGFLEGGWEGMIAGWHGYTSHGAHGVAVAADLKSTQEAINKITKNLNSLSELEVKNLQRLSGAMDELHNEILE |
| | 02/2019 | LDEKVDDLRADTISSQIELAVLLSNEGIINSEDEHLLALERKLKKMLGPSAVEIGNGCFETKHKCNQTCLDRI |
| | | AAGTFDAGEFSLPTFDSLNITAASLNDDGLDNHTILLYYSTAASSLAVTLMIAIFVVYMVSRDNVSCSICL |
| 101 | DNA sequence | atgaaggccatcatcgtgctgctgatggtggtcaccagcaacgccgacagaatctgcaccggcatcaccagca |
| | encoding an | gcaacagccctcacgtggtcaagacagccacacagggcgaagtgaatgtgaccggcgtgatccctctgaccac |
| | influenza HA | cacacctaccaagagccacttcgccaacctgaagggcaccgagacaagaggcaagctgtgccccaagtgcctg |
| | protein of | aactgcaccgatctggatgtggccctgggcagacctaagtgtaccggcaagatccctagcgccagagtgtcca |
| | B/Washington/ | tcctgcacgaagtgcggcctgtgaccagcggctgctttcccattatgcacgaccggaccaagatcagacagct |
| | 02/2019 | gcccaatctgctgcggggctatgaacatgtgcggctgagcacccacaacgtgatcaacgccgaagatgcccct |
| | | ggcagaccatacgagatcggcacatctggctcttgccccaacattaccaacggcaacggcttcttcgccacca |
| | | tggcttgggccgtgcctaagaacaagaccgccaccaatccactgaccatcgaggtgccctacatctgtaccga |
| | | aggcgaggaccagatcaccgtgtggggcttccacagcgacaacgagacacagatggccaaactgtacggcgac |
| | | agcaagccccagaagttcaccagctctgccaacggcgtgaccacacactacgtgtcccagatcggcggcttcc |
| | | ccaatcagacagaagatggcggactgccccagagcggaagaatcgtggtggactacatggtgcagaagtccgg |
| | | caagaccggcacaatcacataccagcggggaatcctgctgcctcagaaagtttggtgcgccagcggccggtcc |
| | | aaagtgatcaaaggatcactgcctctgatcggcgaggccgattgtctgcacgagaaatacggcggcctgaaca |
| | | agagcaagccctactacacaggcgagcacgccaaggccatcggcaactgtcctatctgggtcaagacccctct |
| | | gaagctggccaacggcaccaagtatagacctccagccaagctgctgaaagagcggggcttctttggagctatc |
| | | gccggctttcttgaaggcggctgggagggaatgattgccggctggcatggctacacatctcatggcgcacatg |
| | | gcgtggcagtggccgctgatctgaagtctacacaagaggccatcaacaagatcaccaagaacctgaacagcct |
| | | gagcgagctggaagtgaagaacctgcagagactgtccggcgccatggacgagctgcacaacgagatcctggaa |
| | | ctggacgagaaggtggacgacctgagagccgataccatctccagccagattgagctggcagtgctgctgtcca |
| | | acgagggcatcatcaacagcgaggacgagcatctgctggccctggaacggaagctgaagaagatgctgggccc |
| | | aagcgccgtggaaatcggcaatggctgcttcgagacaaagcacaagtgcaaccagacctgcctggacagaatt |
| | | gccgccggaacatttgacgccggcgagtttagcctgcctaccttcgacagcctgaacatcacagccgccagcc |
| | | tgaatgacgacggcctggacaatcacaccatcctgctgtactactccaccgccgcatcttctctggccgtgac |
| | | actgatgatcgccatctttgtggtgtacatggtgtccagagacaacgtgtcctgcagcatctgcctgtagtga |
| 102 | RNA sequence | augaaggccaucaucgugcugcugaugguggucaccagcaacgccgacagaaucugcaccggcaucaccagca |
| | encoding an | gcaacagcccucacguggucaagacagccacacagggcgaagugaaugugaccggcgugaucccucugaccac |
| | influenza HA | cacaccuaccaagagccacuucgccaaccugaagggcaccgagacaagaggcaagcugugccccaagugccug |
| | protein of | aacugcaccgaucuggauguggcccugggcagaccuaaguguaccggcaagaucccuagcgccagagugucca |
| | B/Washington/ | uccugcacgaagugcggccugugaccagcggcugcuuucccauuaugcacgaccggaccaagaucagacagcu |
| | 02/2019 | gcccaaucugcugcggggcuaugaacaugugcggcugagcacccacaacgugaucaacgccgaagaugccccu |
| | | ggcagaccauacgagaucggcacaucuggcucuugccccaacauuaccaacggcaacggcuucuucgccacca |
| | | uggcuugggccgugccuaagaacaagaccgccaccaauccacugaccaucgaggugcccuacaucuguaccga |
| | | aggcgaggaccagaucaccguguggggcuuccacagcgacaacgagacacagauggccaaacuguacggcgac |
| | | agcaagccccagaaguucaccagcucugccaacggcgugaccacacacuacgugucccagaucggcggcuucc |
| | | ccaaucagacagaagauggcggacugccccagagcggaagaaucgugguggacuacauggugcagaaguccgg |
| | | caagaccggcacaaucacauaccagcggggaauccugcugccucagaaaguuuggugcgccagcggccggucc |
| | | aaagugaucaaaggaucacugccucugaucggcgaggccgauugucugcacgagaaauacggcggccugaaca |
| | | agagcaagcccuacuacacaggcgagcacgccaaggccaucggcaacuguccuaucugggucaagaccccucu |
| | | gaagcuggccaacggcaccaaguauagaccuccagccaagcugcugaaagagcggggcuucuuuggagcuauc |
| | | gccggcuuucuugaaggcggcugggagggaaugauugccggcuggcauggcuacacaucucauggcgcacaug |
| | | gcguggcaguggccgcugaucugaagucuacacaagaggccaucaacaagaucaccaagaaccugaacagccu |
| | | gagcgagcuggaagugaagaaccugcagagacuguccggcgccauggacgagcugcacaacgagauccuggaa |
| | | cuggacgagaagguggacgaccugagagccgauaccaucuccagccagauugagcuggcagugcugcugucca |
| | | acgagggcaucaucaacagcgaggacgagcaucugcuggcccuggaacggaagcugaagaagaugcugggccc |
| | | aagcgccguggaaaucggcaauggcugcuucgagacaaagcacaagugcaaccagaccugccuggacagaauu |
| | | gccgccggaacauuugacgccggcgaguuuagccugccuaccuucgacagccugaacaucacagccgccagcc |
| | | ugaaugacgacggccuggacaaucacaccauccugcuguacuacuccaccgccgcaucuucucuggccgugac |
| | | acugaugaucgccaucuuugugguguacaugguguccagagacaacguguccugcagcaucugccuguaguga |
| 103 | Full length | agaataaactagtattcttctggtccccacagactcagagagaacccgccaccatgaaggccatcatcgtgct |
| | DNA construct | gctgatggtggtcaccagcaacgccgacagaatctgcaccggcatcaccagcagcaacagccctcacgtggtc |
| | sequence | aagacagccacacagggcgaagtgaatgtgaccggcgtgatccctctgaccaccacacctaccaagagccact |
| | encoding | tcgccaacctgaagggcaccgagacaagaggcaagctgtgccccaagtgcctgaactgcaccgatctggatgt |
| | an HA | ggccctgggcagacctaagtgtaccggcaagatccctagcgccagagtgtccatcctgcacgaagtgcggcct |
| | protein of | gtgaccagcggctgctttcccattatgcacgaccggaccaagatcagacagctgcccaatctgctgcggggct |
| | B/Washington/ | atgaacatgtgcggctgagcacccacaacgtgatcaacgccgaagatgcccctggcagaccatacgagatcgg |
| | 02/2019 | cacatctggctcttgccccaacattaccaacggcaacggcttcttcgccaccatggcttgggccgtgcctaag |
| | | aacaagaccgccaccaatccactgaccatcgaggtgccctacatctgtaccgaaggcgaggaccagatcaccg |
| | | tgtggggcttccacagcgacaacgagacacagatggccaaactgtacggcgacagcaagccccagaagttcac |
| | | cagctctgccaacggcgtgaccacacactacgtgtcccagatcggcggcttccccaatcagacagaagatggc |
| | | ggactgccccagagcggaagaatcgtggtggactacatggtgcagaagtccggcaagaccggcacaatcacat |
| | | accagcggggaatcctgctgcctcagaaagtttggtgcgccagcggccggtccaaagtgatcaaaggatcact |
| | | gcctctgatcggcgaggccgattgtctgcacgagaaatacggcggcctgaacaagagcaagccctactacaca |
| | | ggcgagcacgccaaggccatcggcaactgtcctatctgggtcaagacccctctgaagctggccaacggcacca |
| | | agtatagacctccagccaagctgctgaaagagcggggcttctttggagctatcgccggctttcttgaaggcgg |
| | | ctgggagggaatgattgccggctggcatggctacacatctcatggcgcacatggcgtggcagtggccgctgat |
| | | ctgaagtctacacaagaggccatcaacaagatcaccaagaacctgaacagcctgagcgagctggaagtgaaga |
| | | acctgcagagactgtccggcgccatggacgagctgcacaacgagatcctggaactggacgagaaggtggacga |
| | | cctgagagccgataccatctccagccagattgagctggcagtgctgctgtccaacgagggcatcatcaacagc |
| | | gaggacgagcatctgctggccctggaacggaagctgaagaagatgctgggcccaagcgccgtggaaatcggca |
| | | atggctgcttcgagacaaagcacaagtgcaaccagacctgcctggacagaattgccgccggaacatttgacgc |
| | | cggcgagtttagcctgcctaccttcgacagcctgaacatcacagccgccagcctgaatgacgacggcctggac |
| | | aatcacaccatcctgctgtactactccaccgccgcatcttctctggccgtgacactgatgatcgccatctttg |
| | | tggtgtacatggtgtccagagacaacgtgtcctgcagcatctgcctgtagtgactcgagctggtactgcatgc |
| | | acgcaatgctagctgcccctttcccgtcctgggtaccccgagtctcccccgacctcgggtcccaggtatgctc |
| | | ccacctccacctgccccactcaccacctctgctagttccagacacctcccaagcacgcagcaatgcagctcaa |
| | | aacgcttagcctagccacacccccacgggaaacagcagtgattaacctttagcaataaacgaaagtttaacta |
| | | agctatactaaccccagggttggtcaatttcgtgccagccacaccctggagctagcaaaaaaaaaaaaaaaaa |
| | | aaaaaaaaaaaaagcatatgactaaaaaaaaaaaaaaaaaaaaaaaaaaaaaaaaaaaaaaaaaaaaaaaaaa |
| | | aaaaaaaaaaaaaaaaaaaa |
| 104 | Full length | agaauaaacuaguauucuucugguccccacagacucagagagaacccgccaccaugaaggccaucaucgugcu |
| | RNA construct | gcugaugguggucaccagcaacgccgacagaaucugcaccggcaucaccagcagcaacagcccucacgugguc |
| | sequence | aagacagccacacagggcgaagugaaugugaccggcgugaucccucugaccaccacaccuaccaagagccacu |
| | encoding | ucgccaaccugaagggcaccgagacaagaggcaagcugugccccaagugccugaacugcaccgaucuggaugu |
| | an HA | ggcccugggcagaccuaaguguaccggcaagaucccuagcgccagaguguccauccugcacgaagugcggccu |
| | protein of | gugaccagcggcugcuuucccauuaugcacgaccggaccaagaucagacagcugcccaaucugcugcggggcu |
| | B/Washington/ | augaacaugugcggcugagcacccacaacgugaucaacgccgaagaugccccuggcagaccauacgagaucgg |
| | 02/2019 | cacaucuggcucuugccccaacauuaccaacggcaacggcuucuucgccaccauggcuugggccgugccuaag |
| | | aacaagaccgccaccaauccacugaccaucgaggugcccuacaucuguaccgaaggcgaggaccagaucaccg |
| | | uguggggcuuccacagcgacaacgagacacagauggccaaacuguacggcgacagcaagccccagaaguucac |
| | | cagcucugccaacggcgugaccacacacuacgugucccagaucggcggcuuccccaaucagacagaagauggc |
| | | ggacugccccagagcggaagaaucgugguggacuacauggugcagaaguccggcaagaccggcacaaucacau |
| | | accagcggggaauccugcugccucagaaaguuuggugcgccagcggccgguccaaagugaucaaaggaucacu |
| | | gccucugaucggcgaggccgauugucugcacgagaaauacggcggccugaacaagagcaagcccuacuacaca |
| | | ggcgagcacgccaaggccaucggcaacuguccuaucugggucaagaccccucugaagcuggccaacggcacca |
| | | aguauagaccuccagccaagcugcugaaagagcggggcuucuuuggagcuaucgccggcuuucuugaaggcgg |
| | | cugggagggaaugauugccggcuggcauggcuacacaucucauggcgcacauggcguggcaguggccgcugau |
| | | cugaagucuacacaagaggccaucaacaagaucaccaagaaccugaacagccugagcgagcuggaagugaaga |
| | | accugcagagacuguccggcgccauggacgagcugcacaacgagauccuggaacuggacgagaagguggacga |
| | | ccugagagccgauaccaucuccagccagauugagcuggcagugcugcuguccaacgagggcaucaucaacagc |
| | | gaggacgagcaucugcuggcccuggaacggaagcugaagaagaugcugggcccaagcgccguggaaaucggca |
| | | auggcugcuucgagacaaagcacaagugcaaccagaccugccuggacagaauugccgccggaacauuugacgc |
| | | cggcgaguuuagccugccuaccuucgacagccugaacaucacagccgccagccugaaugacgacggccuggac |
| | | aaucacaccauccugcuguacuacuccaccgccgcaucuucucuggccgugacacugaugaucgccaucuuug |
| | | ugguguacaugguguccagagacaacguguccugcagcaucugccuguagugacucgagcugguacugcaugc |
| | | acgcaaugcuagcugccccuuucccguccuggguaccccgagucucccccgaccucgggucccagguaugcuc |
| | | ccaccuccaccugccccacucaccaccucugcuaguuccagacaccucccaagcacgcagcaaugcagcucaa |
| | | aacgcuuagccuagccacacccccacgggaaacagcagugauuaaccuuuagcaauaaacgaaaguuuaacua |
| | | agcuauacuaaccccaggguuggucaauuucgugccagccacacccuggagcuagcaaaaaaaaaaaaaaaaa |
| | | aaaaaaaaaaaaagcauaugacuaaaaaaaaaaaaaaaaaaaaaaaaaaaaaaaaaaaaaaaaaaaaaaaaaa |
| | | aaaaaaaaaaaaaaaaaaaa |

TABLE 22
Sequence of one embodiment of a B/Phuket/3073/2013-specific Influenza RNA vaccine

| SEQ | | |
|---|---|---|
| ID | Brief | |
| NO. | Description | Sequence |
| 105 | Amino acid | MKAIIVLLMVVTSNADRICTGITSSNSPHVVKTATQGEVNVTGVIPLTTTPTKSYFANLKGTRTRGKLCPDCL |
| | sequence of | NCTDLDVALGRPMCVGTTPSAKASILHEVRPVTSGCFPIMHDRTKIRQLPNLLRGYEKIRLSTQNVIDAEKAP |
| | RNA-encoded | GGPYRLGTSGSCPNATSKIGFFATMAWAVPKDNYKNATNPLTVEVPYICTEGEDQITVWGFHSDNKTQMKSLY |
| | Influenza HA | GDSNPQKFTSSANGVTTHYVSQIGDFPDQTEDGGLPQSGRIVVDYMMQKPGKTGTIVYQRGVLLPQKVWCASG |
| | protein of | RSKVIKGSLPLIGEADCLHEEYGGLNKSKPYYTGKHAKAIGNCPIWVKTPLKLANGTKYRPPAKLLKERGFFG |
| | B/Phuket/ | AIAGFLEGGWEGMIAGWHGYTSHGAHGVAVAADLKSTQEAINKITKNLNSLSELEVKNLQRLSGAMDELHNEI |
| | 3073/2013 | LELDEKVDDLRADTISSQIELAVLLSNEGIINSEDEHLLALERKLKKMLGPSAVDIGNGCFETKHKCNQTCLD |
| | | RIAAGTFNAGEFSLPTFDSLNITAASLNDDGLDNHTILLYYSTAASSLAVTLMLAIFIVYMVSRDNVSCSICL |
| 106 | DNA sequence | atgaaggccatcatcgtgctgctgatggtggtcaccagcaacgccgacagaatctgcaccggcatcaccagca |
| | encoding an | gcaacagccctcacgtggtcaagacagccacacagggcgaagtgaatgtgaccggcgtgatccctctgaccac |
| | influenza HA | cacacctaccaagagctacttcgccaacctgaagggcaccagaaccagaggcaagctgtgccccgattgcctg |
| | protein of | aactgcaccgatctggatgtggccctgggcagacctatgtgcgtgggaacaacacctagcgccaaggccagca |
| | B/Phuket/ | tcctgcatgaagtgcggcctgtgaccagcggctgcttccctattatgcacgaccggaccaagatcagacagct |
| | 3073/2013 | gcccaatctgctgcggggctacgagaagatcaggctgagcacccagaacgtgatcgacgccgaaaaagctcct |
| | | ggcggcccttacagactgggcacatctggctcttgccccaacgctacaagcaagatcggcttcttcgccacca |
| | | tggcctgggccgtgcctaaggacaactacaagaacgccaccaatcctctgaccgtggaagtgccctacatctg |
| | | taccgaaggcgaggaccagatcaccgtgtggggcttccacagcgacaacaagacccagatgaagtccctgtac |
| | | ggcgacagcaaccctcagaagtttaccagcagcgccaacggcgtgaccacacactatgtgtcccagatcggcg |
| | | acttccccgaccagacagaagatggcggactgcctcagagcggcagaatcgtggtggactacatgatgcagaa |
| | | gcccggcaagaccggcaccatcgtgtatcagagaggcgtcctgctgccacagaaagtttggtgcgccagcggc |
| | | cggtccaaagtgatcaaaggatcactgcctctgatcggcgaggccgactgtctgcacgaagaatatggcggcc |
| | | tgaacaagagcaagccctactacacaggcaagcacgccaaagccatcggcaactgccctatctgggtcaagac |
| | | ccctctgaagctggccaacggcaccaagtatagacctccagccaagctgctgaaagagcggggcttctttgga |
| | | gctatcgccggctttcttgaaggcggctgggagggaatgattgccggctggcatggctacacatctcatggcg |
| | | cacatggcgtggcagtggccgctgatctgaagtctacacaagaggccatcaacaagatcaccaagaacctgaa |
| | | cagcctgagcgagctggaagtgaagaacctgcagagactgtccggcgccatggacgagctgcacaacgagatc |
| | | ctggaactggacgagaaggtggacgacctgagagccgataccatctccagccagattgagctggcagtgctgc |
| | | tgtccaacgagggcatcatcaacagcgaggacgagcatctgctggccctggaacggaagctgaagaagatgct |
| | | gggacccagcgccgtggatatcggcaatggctgcttcgagacaaagcacaagtgcaaccagacctgcctggac |
| | | agaattgccgccggaacctttaacgccggcgagtttagcctgcctaccttcgacagcctgaacatcacagccg |
| | | ccagcctgaatgacgacggcctggacaatcacaccatcctgctgtactactccaccgccgcatcttctctggc |
| | | cgtgacactgatgctggctatcttcatcgtgtacatggtgtccagagacaacgtgtcctgcagcatctgcctg |
| | | tgatga |
| 107 | RNA sequence | augaaggccaucaucgugcugcugaugguggucaccagcaacgccgacagaaucugcaccggcaucaccagca |
| | encoding an | gcaacagcccucacguggucaagacagccacacagggcgaagugaaugugaccggcgugaucccucugaccac |
| | influenza HA | cacaccuaccaagagcuacuucgccaaccugaagggcaccagaaccagaggcaagcugugccccgauugccug |
| | protein of | aacugcaccgaucuggauguggcccugggcagaccuaugugcgugggaacaacaccuagcgccaaggccagca |
| | B/Phuket/ | uccugcaugaagugcggccugugaccagcggcugcuucccuauuaugcacgaccggaccaagaucagacagcu |
| | 3073/2013 | gcccaaucugcugcggggcuacgagaagaucaggcugagcacccagaacgugaucgacgccgaaaaagcuccu |
| | | ggcggcccuuacagacugggcacaucuggcucuugccccaacgcuacaagcaagaucggcuucuucgccacca |
| | | uggccugggccgugccuaaggacaacuacaagaacgccaccaauccucugaccguggaagugcccuacaucug |
| | | uaccgaaggcgaggaccagaucaccguguggggcuuccacagcgacaacaagacccagaugaagucccuguac |
| | | ggcgacagcaacccucagaaguuuaccagcagcgccaacggcgugaccacacacuaugugucccagaucggcg |
| | | acuuccccgaccagacagaagauggcggacugccucagagcggcagaaucgugguggacuacaugaugcagaa |
| | | gcccggcaagaccggcaccaucguguaucagagaggcguccugcugccacagaaaguuuggugcgccagcggc |
| | | cgguccaaagugaucaaaggaucacugccucugaucggcgaggccgacugucugcacgaagaauauggcggcc |
| | | ugaacaagagcaagcccuacuacacaggcaagcacgccaaagccaucggcaacugcccuaucugggucaagac |
| | | cccucugaagcuggccaacggcaccaaguauagaccuccagccaagcugcugaaagagcggggcuucuuugga |
| | | gcuaucgccggcuuucuugaaggcggcugggagggaaugauugccggcuggcauggcuacacaucucauggcg |
| | | cacauggcguggcaguggccgcugaucugaagucuacacaagaggccaucaacaagaucaccaagaaccugaa |
| | | cagccugagcgagcuggaagugaagaaccugcagagacuguccggcgccauggacgagcugcacaacgagauc |
| | | cuggaacuggacgagaagguggacgaccugagagccgauaccaucuccagccagauugagcuggcagugcugc |
| | | uguccaacgagggcaucaucaacagcgaggacgagcaucugcuggcccuggaacggaagcugaagaagaugcu |
| | | gggacccagcgccguggauaucggcaauggcugcuucgagacaaagcacaagugcaaccagaccugccuggac |
| | | agaauugccgccggaaccuuuaacgccggcgaguuuagccugccuaccuucgacagccugaacaucacagccg |
| | | ccagccugaaugacgacggccuggacaaucacaccauccugcuguacuacuccaccgccgcaucuucucuggc |
| | | cgugacacugaugcuggcuaucuucaucguguacaugguguccagagacaacguguccugcagcaucugccug |
| | | ugauga |
| 108 | Full length | agaataaactagtattcttctggtccccacagactcagagagaacccgccaccatgaaggccatcatcgtgct |
| | DNA construct | gctgatggtggtcaccagcaacgccgacagaatctgcaccggcatcaccagcagcaacagccctcacgtggtc |
| | sequence | aagacagccacacagggcgaagtgaatgtgaccggcgtgatccctctgaccaccacacctaccaagagctact |
| | encoding | tcgccaacctgaagggcaccagaaccagaggcaagctgtgccccgattgcctgaactgcaccgatctggatgt |
| | an HA | ggccctgggcagacctatgtgcgtgggaacaacacctagcgccaaggccagcatcctgcatgaagtgcggcct |
| | protein of | gtgaccagcggctgcttccctattatgcacgaccggaccaagatcagacagctgcccaatctgctgcggggct |
| | B/Phuket/ | acgagaagatcaggctgagcacccagaacgtgatcgacgccgaaaaagctcctggcggcccttacagactggg |
| | 3073/2013 | cacatctggctcttgccccaacgctacaagcaagatcggcttcttcgccaccatggcctgggccgtgcctaag |
| | | gacaactacaagaacgccaccaatcctctgaccgtggaagtgccctacatctgtaccgaaggcgaggaccaga |
| | | tcaccgtgtggggcttccacagcgacaacaagacccagatgaagtccctgtacggcgacagcaaccctcagaa |
| | | gtttaccagcagcgccaacggcgtgaccacacactatgtgtcccagatcggcgacttccccgaccagacagaa |
| | | gatggcggactgcctcagagcggcagaatcgtggtggactacatgatgcagaagcccggcaagaccggcacca |
| | | tcgtgtatcagagaggcgtcctgctgccacagaaagtttggtgcgccagcggccggtccaaagtgatcaaagg |
| | | atcactgcctctgatcggcgaggccgactgtctgcacgaagaatatggcggcctgaacaagagcaagccctac |
| | | tacacaggcaagcacgccaaagccatcggcaactgccctatctgggtcaagacccctctgaagctggccaacg |
| | | gcaccaagtatagacctccagccaagctgctgaaagagcggggcttctttggagctatcgccggctttcttga |
| | | aggcggctgggagggaatgattgccggctggcatggctacacatctcatggcgcacatggcgtggcagtggcc |
| | | gctgatctgaagtctacacaagaggccatcaacaagatcaccaagaacctgaacagcctgagcgagctggaag |
| | | tgaagaacctgcagagactgtccggcgccatggacgagctgcacaacgagatcctggaactggacgagaaggt |
| | | ggacgacctgagagccgataccatctccagccagattgagctggcagtgctgctgtccaacgagggcatcatc |
| | | aacagcgaggacgagcatctgctggccctggaacggaagctgaagaagatgctgggacccagcgccgtggata |
| | | tcggcaatggctgcttcgagacaaagcacaagtgcaaccagacctgcctggacagaattgccgccggaacctt |
| | | taacgccggcgagtttagcctgcctaccttcgacagcctgaacatcacagccgccagcctgaatgacgacggc |
| | | ctggacaatcacaccatcctgctgtactactccaccgccgcatcttctctggccgtgacactgatgctggcta |
| | | tcttcatcgtgtacatggtgtccagagacaacgtgtcctgcagcatctgcctgtgatgactcgagctggtact |
| | | gcatgcacgcaatgctagctgcccctttcccgtcctgggtaccccgagtctcccccgacctcgggtcccaggt |
| | | atgctcccacctccacctgccccactcaccacctctgctagttccagacacctcccaagcacgcagcaatgca |
| | | gctcaaaacgcttagcctagccacacccccacgggaaacagcagtgattaacctttagcaataaacgaaagtt |
| | | taactaagctatactaaccccagggttggtcaatttcgtgccagccacaccctggagctagcaaaaaaaaaaa |
| | | aaaaaaaaaaaaaaaaaaagcatatgactaaaaaaaaaaaaaaaaaaaaaaaaaaaaaaaaaaaaaaaaaaaa |
| | | aaaaaaaaaaaaaaaaaaaaaaaaaa |
| 109 | Full length | agaauaaacuaguauucuucugguccccacagacucagagagaacccgccaccaugaaggccaucaucgugcu |
| | RNA construct | gcugaugguggucaccagcaacgccgacagaaucugcaccggcaucaccagcagcaacagcccucacgugguc |
| | sequence | aagacagccacacagggcgaagugaaugugaccggcgugaucccucugaccaccacaccuaccaagagcuacu |
| | encoding | ucgccaaccugaagggcaccagaaccagaggcaagcugugccccgauugccugaacugcaccgaucuggaugu |
| | an HA | ggcccugggcagaccuaugugcgugggaacaacaccuagcgccaaggccagcauccugcaugaagugcggccu |
| | protein of | gugaccagcggcugcuucccuauuaugcacgaccggaccaagaucagacagcugcccaaucugcugcggggcu |
| | B/Phuket/ | acgagaagaucaggcugagcacccagaacgugaucgacgccgaaaaagcuccuggcggcccuuacagacuggg |
| | 3073/2013 | cacaucuggcucuugccccaacgcuacaagcaagaucggcuucuucgccaccauggccugggccgugccuaag |
| | | gacaacuacaagaacgccaccaauccucugaccguggaagugcccuacaucuguaccgaaggcgaggaccaga |
| | | ucaccguguggggcuuccacagcgacaacaagacccagaugaagucccuguacggcgacagcaacccucagaa |
| | | guuuaccagcagcgccaacggcgugaccacacacuaugugucccagaucggcgacuuccccgaccagacagaa |
| | | gauggcggacugccucagagcggcagaaucgugguggacuacaugaugcagaagcccggcaagaccggcacca |
| | | ucguguaucagagaggcguccugcugccacagaaaguuuggugcgccagcggccgguccaaagugaucaaagg |
| | | aucacugccucugaucggcgaggccgacugucugcacgaagaauauggcggccugaacaagagcaagcccuac |
| | | uacacaggcaagcacgccaaagccaucggcaacugcccuaucugggucaagaccccucugaagcuggccaacg |
| | | gcaccaaguauagaccuccagccaagcugcugaaagagcggggcuucuuuggagcuaucgccggcuuucuuga |
| | | aggcggcugggagggaaugauugccggcuggcauggcuacacaucucauggcgcacauggcguggcaguggcc |
| | | gcugaucugaagucuacacaagaggccaucaacaagaucaccaagaaccugaacagccugagcgagcuggaag |
| | | ugaagaaccugcagagacuguccggcgccauggacgagcugcacaacgagauccuggaacuggacgagaaggu |
| | | ggacgaccugagagccgauaccaucuccagccagauugagcuggcagugcugcuguccaacgagggcaucauc |
| | | aacagcgaggacgagcaucugcuggcccuggaacggaagcugaagaagaugcugggacccagcgccguggaua |
| | | ucggcaauggcugcuucgagacaaagcacaagugcaaccagaccugccuggacagaauugccgccggaaccuu |
| | | uaacgccggcgaguuuagccugccuaccuucgacagccugaacaucacagccgccagccugaaugacgacggc |
| | | cuggacaaucacaccauccugcuguacuacuccaccgccgcaucuucucuggccgugacacugaugcuggcua |
| | | ucuucaucguguacaugguguccagagacaacguguccugcagcaucugccugugaugacucgagcugguacu |
| | | gcaugcacgcaaugcuagcugccccuuucccguccuggguaccccgagucucccccgaccucgggucccaggu |
| | | augcucccaccuccaccugccccacucaccaccucugcuaguuccagacaccucccaagcacgcagcaaugca |
| | | gcucaaaacgcuuagccuagccacacccccacgggaaacagcagugauuaaccuuuagcaauaaacgaaaguu |
| | | uaacuaagcuauacuaaccccaggguuggucaauuucgugccagccacacccuggagcuagcaaaaaaaaaaa |
| | | aaaaaaaaaaaaaaaaaaagcauaugacuaaaaaaaaaaaaaaaaaaaaaaaaaaaaaaaaaaaaaaaaaaaa |
| | | aaaaaaaaaaaaaaaaaaaaaaaaaa |

Tables 8, and 10-16D show amino acid sequences of SARS-CoV-2 S proteins encoded by RNAs described herein from different variants with 2 proline substitutions at positions corresponding to K986P and V987P of SEQ ID NO: 1. In some embodiments, an RNA described herein encodes a SARS-CoV-2 S protein as described herein (e.g., as described in Tables 8, 10-16D) without the 2 proline substitutions at positions corresponding to K986P and V987P of SEQ ID NO: 1. In some embodiments, an RNA described herein encodes a SARS-CoV-2 S protein comprising one or more mutations characteristic of a variant described herein (e.g., as described in Table 2), and further comprising at least four (including, e.g., at least five, at least six, or more) proline mutations. In some embodiments, at least four of such proline mutations include mutations at positions corresponding to residues 817, 892, 899, and 942 of SEQ ID NO: 1, e.g., as described in WO 2021243122 A2, the entire contents of which are incorporated herein by reference in its entirety. In some embodiments, such a SARS-CoV-2 S protein comprising proline substitutions at positions corresponding to residues 817, 892, 899, and 942 of SEQ ID NO: 1, may further comprise proline substitutions at positions corresponding to residues 986 and 987 of SEQ ID NO: 1.

In some embodiments, an RNA encoding an antigen described herein (e.g., as shown in Tables 8, and 10-22) can be an unmodified RNA. In some embodiments, an RNA described herein (e.g., as shown in Tables 8, and 10-22) can be a modified RNA. For example, in some embodiments, an RNA described herein (e.g., as shown in Tables 8, and 10-22) can comprise modified uridines (e.g., as described herein) in place of uridines. In some embodiments, an RNA described herein (e.g., as shown in Tables 8, and 10-22) can be a self-amplifying RNA, e.g., using a construct as described herein.

Self-Amplifying RNA (saRNA)

The active principle of a self-amplifying RNA (saRNA) drug substance is a single-stranded RNA, which can self-amplify upon entering a cell, and an encoded antigen can be translated thereafter. In contrast to mRNA which preferably code for a single protein, the coding region of saRNA contains two open reading frames (ORFs). The 5′-ORF encodes an RNA-dependent RNA polymerase such as Venezuelan equine encephalitis virus (VEEV) RNA-dependent RNA polymerase (replicase). The replicase ORF is followed 3′ by a subgenomic promoter and a second ORF encoding the antigen. Furthermore, saRNA UTRs contain 5′ and 3′ conserved sequence elements (CSEs) required for self-amplification. In some embodiments, an saRNA contains common structural elements optimized for maximal efficacy of the RNA as the uRNA (5′-cap, 5′-UTR, 3′-UTR, poly(A)-tail). In some embodiments, saRNA preferably contains uridine. The preferred 5′ cap structure is beta-S-ARCA(D1) (m₂^{7,2′-O}GppSpG) for saRNA.

Cytoplasmic delivery of saRNA initiates an alphavirus-like life cycle. However, saRNA does not encode for alphaviral structural proteins that are required for genome packaging or cell entry; therefore generation of replication competent viral particles is very unlikely or not possible. Replication does not involve any intermediate steps that generate DNA. The use/uptake of saRNA therefore poses no risk of genomic integration or other permanent genetic modification within a target cell. Furthermore, saRNA itself prevents its persistent replication by effectively activating innate immune response via recognition of dsRNA intermediates.

In some embodiments, an saRNA described herein encodes a single antigen (e.g., one HA polypeptide, one NA polypeptide, or one SARS-CoV-2 S polypeptide). In some embodiments, an saRNA utilized in accordance with the present disclosure encodes two or more antigens (e.g., two or more HA polypeptides, two or more NA polypeptides, two or more SARS-CoV-2 S polypeptides, one or more HA polypeptides and one or more NA polypeptides, one or more HA polypeptide and one or more SARS-CoV-2 S polypeptides, or one or more NA polypeptides and one or more SARS-CoV-2 S polypeptides). In some embodiments, an saRNA encodes two HA polypeptides, each from a different influenza strain. In some embodiments, an saRNA encodes two NA polypeptides, each from a different influenza strain. In some embodiments, an saRNA encodes two SARS-CoV-2 S polypeptides, each from a different variant. In some embodiments, an saRNA encodes an HA polypeptide and an NA polypeptide, each from the same influenza strain (e.g., as described in “Pfizer Near-Term Launches+High-Value Pipeline Day”, published Dec. 12, 2022; https_//s28.q4cdn.com/781576035/files/doc_presentation/2022/12/B/Pfizer-Near-Term-Launches-High-Value-Pipeline-Day-Presentation_6 pm_v2.pdf).

In some embodiments, a nucleotide sequence encoding an antigenic polypeptide is located downstream of saRNA Replicase genes. In some embodiments, an saRNA comprises a nucleotide sequence encoding an HA antigen and a nucleotide sequence encoding an NA antigen, where the nucleotide sequence encoding the HA antigen is located upstream of the nucleotide sequence encoding the NA antigen.

In some embodiments, an saRNA platform can provide certain advantages as compared to other RNA platforms. For example, in some embodiments, saRNA can provide increased duration of expression of an antigen, lower dose levels, improved tolerability, and/or increased antigen capacity, while maintaining a robust antibody and T cell response.

Different embodiments of this platform are as follows:

### RBS004.1 (SEQ ID NO: 24; SEQ ID NO: 7)

Structure beta-S-ARCA(D1)-replicase-S1S2-PP-FI-A30L70
Encoded antigen Viral spike protein (S protein) of the SARS-CoV-2 (S1S2 full-length protein, sequence variant)

### RBS004.2 (SEQ ID NO: 25; SEQ ID NO: 7)

Structure beta-S-ARCA(D1)-replicase-S1S2-PP-FI-A30L70
Encoded antigen Viral spike protein (S protein) of the SARS-CoV-2 (S1S2 full-length protein, sequence variant)

### BNT162c1; RBS004.3 (SEQ ID NO: 26; SEQ ID NO: 5)

Structure beta-S-ARCA(D1)-replicase-RBD-GS-Fibritin-FI-A30L70
Encoded antigen Viral spike protein (S protein) of the SARS-CoV-2 (partial sequence, Receptor Binding Domain (RBD) of S1S2 protein)

### RBS004.4 (SEQ ID NO: 27; SEQ ID NO: 28)

Structure beta-S-ARCA(D1)-replicase-RBD-GS-Fibritin-TM-FI-A30L70
Encoded antigen Viral spike protein (S protein) of the SARS-CoV-2 (partial sequence, Receptor Binding Domain (RBD) of S1S2 protein)

In some embodiments, RNA described herein comprises a nucleotide sequence selected from the group consisting of SEQ ID NO: 15, 16, 17, 19, 20, 21, 24, 25, 26, 27, 30, and 32. A particularly preferred vaccine RNA described herein comprises a nucleotide sequence selected from the group consisting of SEQ ID NO: 15, 17, 19, 21, 25, 26, 30, and 32 such as selected from the group consisting of SEQ ID NO: 17, 19, 21, 26, 30, and 32.

In some embodiments, one or more RNA described herein can be partially or completely encapsulated in nanoparticles. In some embodiments, nanoparticles encapsulating one or more RNA comprise lipid nanoparticles, polyplexes (PLX), lipidated polyplexes (LPLX), liposomes, or polysaccharide nanoparticles. In some embodiments, one or more RNA described herein is formulated in lipid nanoparticles (LNP). In one embodiment, an LNP comprises a cationic lipid, a neutral lipid, a steroid, a polymer conjugated lipid; and one or more RNAs. In one embodiment, the cationic lipid is ALC-0315, the neutral lipid is DSPC, the steroid is cholesterol, and the polymer conjugated lipid is ALC-0159. The preferred mode of administration is intramuscular administration, more preferably in aqueous cryoprotectant buffer for intramuscular administration. The drug product is a preferably a preservative-free, sterile dispersion of RNA formulated in lipid nanoparticles (LNP) in aqueous cryoprotectant buffer for intramuscular administration.

In some embodiments, a drug product comprises the components shown below, preferably at the proportions or concentrations shown below:

| Component | Function | Proportion (mol %) |
|---|---|---|
| ALC-0315 [1] | Functional lipid | 47.5 |
| ALC-0159 [2] | Functional lipid | 1.8 |
| DSPC [3] | Structural lipid | 10.0 |
| Cholesterol, synthetic | Structural lipid | 40.7 |
| | | Concentration |
| Component | Function | (mg/mL) |
| Drug Substance | Active | 0.5 |
| ALC-0315 [1] | Functional lipid | 7.17 |
| ALC-0159 [2] | Functional lipid | 0.89 |
| DSPC [3] | Structural lipid | 1.56 |
| Cholesterol, synthetic | Structural lipid | 3.1 |
| Sucrose | Cryoprotectant | 102.69 |
| NaCl | Buffer | 6.0 |
| KCl | Buffer | 0.15 |
| Na₂HPO₄ | Buffer | 1.08 |
| KH₂PO₄ | Buffer | 0.18 |
| Water for injection | Solvent/Vehicle | q.s. |
| | | Concentration |
| Component | Function | (mg/mL) |
| Drug Substance | Active | 1.0 |
| ALC-0315 [1] | Functional lipid | 13.56 |
| ALC-0159 [2] | Functional lipid | 1.77 |
| DSPC [3] | Structural lipid | 3.11 |
| Cholesterol, synthetic | Structural lipid | 6.20 |
| Sucrose | Cryoprotectant | 102.69 |
| NaCl | Buffer | 6.0 |
| KCl | Buffer | 0.15 |
| Na₂HPO₄ | Buffer | 1.08 |
| KH₂PO₄ | Buffer | 0.15 |
| Water for injection | Solvent/Vehicle | q.s. |

[1] ALC-0315 = ((4-hydroxybutyl)azanediyl)bis(hexane-6,1-diyl)bis(2-hexyldecanoate)/6-[N-6-(2-hexyldecanoyloxy)hexyl-N-(4-hydroxybutyl)amino]hexyl 2-hexyldecanoate
[2] ALC-0159 = 2-[(polyethylene glycol)-2000]-N,N-ditetradecylacetamide/2-[2-(ω-methoxy(polyethyleneglycol2000) ethoxy]-N, N-ditetradecylacetamide
[3] DSPC = 1,2-Distearoyl-sn-glycero-3-phosphocholine
q.s. = quantum satis (as much as may suffice)
ALC-0315: 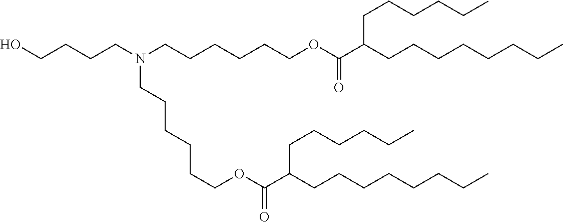 [SMILES: CCCCCCCCC(CCCCCC)C(=O)OCCCCCCN(CCCCO)CCCCCCOC(=O)C(CCCCCC)CCCCCCCC]
ALC-1059: 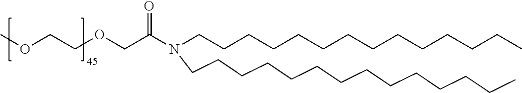
DSPC: 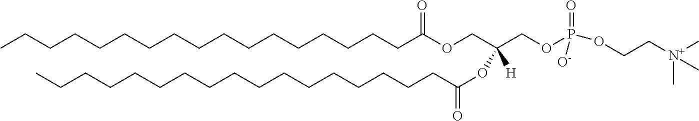 [SMILES: CCCCCCCCCCCCCCCCCC(=O)OC[C@H](COP(=O)([O-])OCC[N+](C)(C)C)OC(=O)CCCCCCCCCCCCCCCCC]
Cholesterol:

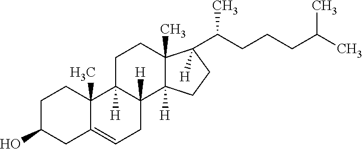

In some embodiments, particles disclosed herein are formulated in a solution comprising 10 mM Tris and 10% sucrose, and optionally having a pH of about 7.4.

In some embodiments, particles disclosed herein are formulated in a solution comprising about 103 mg/ml sucrose, about 0.20 mg/ml tromethamine (Tris base), and about 1.32 mg/ml Tris.

In some embodiments, a composition comprises: (a) about 0.1 mg/mL RNA comprising an open reading frame encoding a polypeptide that comprises a SARS-CoV-2 protein or an immunogenic fragment or variant thereof, (b) about 1.43 mg/ml ALC-0315, (c) about 0.18 mg/ml ALC-0159, (d) about 0.31 mg/ml DSPC, (e) about 0.62 mg/ml cholesterol, (f) about 103 mg/ml sucrose, (g) about 0.20 mg/ml tromethamine (Tris base), (h) about 1.32 mg/ml Tris (hydroxymethyl)aminomethane hydrochloride (Tris HCl), and (i) q.s. water.

In one embodiment, the ratio of mRNA to total lipid (N/P) is between 6.0 and 6.5 such as about 6.0 or about 6.3.

### Nucleic Acid Containing Particles

Nucleic acids described herein such as RNA encoding a vaccine antigen may be administered formulated as particles.

In the context of the present disclosure, the term “particle” relates to a structured entity formed by molecules or molecule complexes. In one embodiment, the term “particle” relates to a micro- or nano-sized structure, such as a micro- or nano-sized compact structure dispersed in a medium. In one embodiment, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In one embodiment, a nucleic acid particle is a nanoparticle.

As used in the present disclosure, “nanoparticle” refers to a particle having an average diameter suitable for parenteral administration. In some embodiments, nanoparticles encapsulating RNAs described eh rein may have an average diameter of about 50-150 nm.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations. In some embodiments, exemplary nanoparticles include lipid nanoparticles, polyplexes (PLX), lipidated polyplexes (LPLX), liposomes, or polysaccharide nanoparticles.

In some embodiments, an LNP comprises one or more cationically ionizable lipids; one or more neutral lipids (e.g., in some embodiments sterol such as, e.g., cholesterol; and/or phospholipids), and one or more polymer-conjugated lipids. In some embodiments, the formulation comprises ALC-0315 (4-hydroxybutyl)azanediyl)bis(hexane-6,1-diyl)bis(2-hexyldecanoate), ALC-0159 (2-[(polyethylene glycol)-2000]-N, N-ditetradecylacetamide), DSPC (1,2-distearoyl-sn-glycero-3-phosphocholine), cholesterol, sucrose, trometamol (Tris), trometamol hydrochloride and water.

RNA particles described herein include nanoparticles. In some embodiments, exemplary nanoparticles include lipid nanoparticles, lipoplex, polyplexes (PLX), lipidated polyplexes (LPLX), liposomes, or polysaccharide nanoparticles. Polyplexes (PLX), polysaccharide nanoparticles, and liposomes, are all delivery technologies that are well known to a person of skill in the art. See, e.g., Lächelt, Ulrich, and Ernst Wagner. “Nucleic acid therapeutics using polyplexes: a journey of 50 years (and beyond)” Chemical reviews 115.19 (2015): 11043-11078; Plucinski, Alexander, Zan Lyu, and Bernhard V K J Schmidt, “Polysaccharide nanoparticles: from fabrication to applications.” Journal of Materials Chemistry B (2021); and Tenchov, Rumiana, et al. “Lipid Nanoparticles—From Liposomes to mRNA Vaccine Delivery, a Landscape of Research Diversity and Advancement,” ACS nano 15.11 (2021): 16982-17015, respectively, the contents of each of which are hereby incorporated by reference herein in their entirety. In some embodiments, the concentration of RNA in a pharmaceutical RNA preparation is about 0.1 mg/ml. In some embodiments, the concentration of RNA in a pharmaceutical RNA preparation is about 30 μg/ml to about 100 μg/ml. In some embodiments, the concentration of RNA in a pharmaceutical RNA preparation is about 50 μg/ml to about 100 μg/ml.

Without intending to be bound by any theory, it is believed that the cationic or cationically ionizable lipid or lipid-like material and/or the cationic polymer combine together with nucleic acid to form aggregates, and this aggregation results in colloidally stable particles.

In one embodiment, particles described herein further comprise at least one lipid or lipid-like material other than a cationic or cationically ionizable lipid or lipid-like material, at least one polymer other than a cationic polymer, or a mixture thereof

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features.

Nucleic acid particles described herein may have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

With respect to RNA lipid particles, the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, RNA lipoplex particles described herein are obtainable by adding RNA to a colloidal liposome dispersion. Using the ethanol injection technique, such colloidal liposome dispersion is, in one embodiment, formed as follows: an ethanol solution comprising lipids, such as cationic lipids and additional lipids, is injected into an aqueous solution under stirring. In one embodiment, RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid such as polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

In some embodiments, a nucleic acid containing particle (e.g., a lipid nanoparticle (LNP)) comprises two or more RNA molecules, each comprising a different nucleic acid sequence. In some embodiments, a nucleic acid containing particle comprises two or more RNA molecules, each encoding a different immunogenic polypeptide or immunogenic fragment thereof. In some embodiments, two or more RNA molecules present in a nucleic acid containing particle comprise: a first RNA molecule encodes an immunogenic polypeptide or immunogenic fragment thereof from a coronavirus and a second RNA molecule encodes an immunogenic polypeptide or immunogenic fragment thereof from an infectious disease pathogen (e.g., virus, bacteria, parasite, etc.). For example, in some embodiments, two or more RNA molecules present in a nucleic acid containing particle comprise: a first RNA molecule encoding an immunogenic polypeptide or immunogenic fragment thereof from a coronavirus (e.g., in some embodiments SARS-CoV-2 Wuhan strain or a variant thereof, e.g., a SARS-CoV-2 having one or more mutations characteristic of an Omicron variant (e.g., a BA.1, BA.4/5, or XBB.1.5 Omicron variant)) and a second RNA molecule encoding an immunogenic polypeptide or immunogenic fragment thereof from an influenza virus. In some embodiments, two or more RNA molecules present in a nucleic acid containing particle comprise: a first RNA molecule encoding an immunogenic polypeptide or immunogenic fragment thereof from a first coronavirus (e.g., as described herein) and a second RNA molecule encoding an immunogenic polypeptide or immunogenic fragment thereof from a second coronavirus (e.g., as described herein). In some embodiments, a first coronavirus is different from a second coronavirus. In some embodiments, a first and/or second coronavirus is independently from a SARS-CoV-2 Wuhan strain or a variant thereof, e.g., a SARS-CoV-2 having one or more mutations characteristic of an Omicron variant (e.g., a BA.1, BA.4/5, or XBB.1.5 Omicron variant).

In some embodiments, two or more RNA molecules present in a nucleic acid containing particle each encode an immunogenic polypeptide or an immunogenic fragment thereof from the same and/or different strains and/or variants of coronavirus (e.g., in some embodiments SARS-CoV-2 strains or variants). For example, in some embodiments, two or more RNA molecules present in a nucleic acid containing particle each encode a different immunogenic polypeptide or immunogenic fragment thereof from a coronavirus membrane protein, a coronavirus nucleocapsid protein, a coronavirus spike protein, a coronavirus non-structural protein and/or a coronavirus accessory protein. In some embodiments, such immunogenic polypeptides or immunogenic fragments thereof may be from the same or a different coronavirus (e.g., in some embodiments a SARS-CoV-2 Wuhan strain or variants thereof, for example, in some embodiments a variant having one or more mutations characteristic of a prevalent variant such as an Omicron variant (e.g., a BA.1, BA.4/5, or XBB.1.5 Omicron variant)). In some embodiments, a nucleic acid containing particle comprises a first RNA molecule encoding a SARS-CoV-2 S protein or an immunogenic fragment thereof from a first strain or variant, and a second RNA molecule encoding a SARS-CoV-2 S protein or an immunogenic fragment thereof from a second strain or variant, wherein the second strain or variant is different from the first strain or variant.

In some embodiments, a nucleic acid containing particle (e.g., in some embodiments an LNP as described herein) comprises a first RNA molecule encoding a SARS-CoV-2 S protein from a Wuhan strain and a second RNA molecule encoding a SARS-CoV-2 S protein comprising one or more mutations that are characteristic of an Omicron variant (e.g., a BA.1, BA.2, BA.3, BA.4, BA.5, or XBB.1.5 Omicron variant).

In some embodiments, a nucleic acid containing particle comprises a first RNA molecule encoding a SARS-CoV-2 S protein from a Wuhan strain and a second RNA molecule encoding a SARS-CoV-2 S protein comprising one or more mutations that are characteristic of an Omicron BA.1 variant. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.1 variant is 1:1. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.1 variant is 1:2. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.1 variant is 1:3.

In some embodiments, a nucleic acid containing particle comprises a first RNA molecule encoding a SARS-CoV-2 S protein from a Wuhan strain and a second RNA molecule encoding a SARS-CoV-2 S protein comprising one or more mutations that are characteristic of an Omicron BA.2 variant. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.2 variant is 1:1. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.2 variant is 1:2. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.2 variant is 1:3.

In some embodiments, a nucleic acid containing particle comprises a first RNA molecule encoding a SARS-CoV-2 S protein from a Wuhan strain and a second RNA molecule encoding a SARS-CoV-2 S protein comprising one or more mutations that are characteristic of an Omicron BA.3 variant. In some embodiments, a nucleic acid containing particle comprises a first RNA molecule encoding a SARS-CoV-2 S protein from a Wuhan strain and a second RNA molecule encoding a SARS-CoV-2 S protein comprising one or more mutations that are characteristic of an Omicron BA.4 or BA.5 variant. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.4 or BA.5 variant is 1:1. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.4 or BA.5 variant is 1:2. In some embodiments, the ratio of the first RNA molecule encoding a SARS-CoV-2 S protein from a Wuhan strain and the second RNA molecule encoding a SARS-CoV-2 S protein comprising one or more mutations that are characteristic of an Omicron BA.4 or BA.5 variant is 1:3.

In some embodiments, a nucleic acid containing particle comprises a first RNA molecule encoding a SARS-CoV-2 S protein from a first Omicron variant and a second RNA molecule encoding a SARS-CoV-2 S protein comprising one or more mutations that are characteristic of a second Omicron variant.

In some embodiments, a nucleic acid containing particle comprises a first RNA molecule encoding a SARS-CoV-2 S protein from a BA.1 Omicron variant and a second RNA molecule encoding a SARS-CoV-2 S protein comprising one or more mutations that are characteristic of a BA.2 Omicron variant. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.2 variant is 1:1. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.2 variant is 1:2. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.2 variant is 1:3.

In some embodiments, a nucleic acid containing particle comprises a first RNA molecule encoding a SARS-CoV-2 S protein from a BA.1 Omicron variant and a second RNA molecule encoding a SARS-CoV-2 S protein comprising one or more mutations that are characteristic of a BA.3 Omicron variant. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.3 variant is 1:1. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.3 variant is 1:2. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.3 variant is 1:3.

In some embodiments, a nucleic acid containing particle comprises a first RNA molecule encoding a SARS-CoV-2 S protein from a BA.1 Omicron variant and a second RNA molecule encoding a SARS-CoV-2 S protein comprising one or more mutations that are characteristic of a BA.4 or BA.5 Omicron variant. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.4 or BA.5 variant is 1:1. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.4 or BA.5 variant is 1:2. In some embodiments, the ratio of the first RNA molecule encoding a SARS-CoV-2 S protein comprising one or more mutations characteristic of a BA.1 Omicron variant strain and the second RNA molecule encoding a SARS-CoV-2 S protein comprising one or more mutations that are characteristic of an Omicron BA.4 or BA.5 variant is 1:3.

In some embodiments, a nucleic acid containing particle comprises a first RNA molecule encoding a SARS-CoV-2 S protein from a BA.2 Omicron variant and a second RNA molecule encoding a SARS-CoV-2 S protein comprising one or more mutations that are characteristic of a BA.3 Omicron variant. In some embodiments, the ratio of the first RNA molecule encoding a SARS-CoV-2 S protein comprising one or more mutations characteristic of a BA.2 Omicron variant strain and the second RNA molecule encoding a SARS-CoV-2 S protein comprising one or more mutations that are characteristic of an Omicron BA.3 variant is 1:1. In some embodiments, the ratio of the first RNA molecule encoding a SARS-CoV-2 S protein comprising one or more mutations characteristic of a BA.2 Omicron variant strain and the second RNA molecule encoding a SARS-CoV-2 S protein comprising one or more mutations that are characteristic of an Omicron BA.3 variant is 1:2. In some embodiments, the ratio of the first RNA molecule encoding a SARS-CoV-2 S protein comprising one or more mutations characteristic of a BA.2 Omicron variant strain and the second RNA molecule encoding a SARS-CoV-2 S protein comprising one or more mutations that are characteristic of an Omicron BA.3 variant is 1:3.

In some embodiments, a nucleic acid containing particle comprises a first RNA molecule encoding a SARS-CoV-2 S protein from a BA.2 Omicron variant and a second RNA molecule encoding a SARS-CoV-2 S protein comprising one or more mutations that are characteristic of a BA.4 or BA.5 Omicron variant. In some embodiments, the ratio of the first RNA molecule encoding a SARS-CoV-2 S protein comprising one or more mutations characteristic of a BA.2 Omicron variant strain and the second RNA molecule encoding a SARS-CoV-2 S protein comprising one or more mutations that are characteristic of an Omicron BA.4 or BA.5 variant is 1:1. In some embodiments, the ratio of the first RNA molecule encoding a SARS-CoV-2 S protein comprising one or more mutations characteristic of a BA.2 Omicron variant strain and the second RNA molecule encoding a SARS-CoV-2 S protein comprising one or more mutations that are characteristic of an Omicron BA.4 or BA.5 variant is 1:2. In some embodiments, the ratio of the first RNA molecule encoding a SARS-CoV-2 S protein comprising one or more mutations characteristic of a BA.2 Omicron variant strain and the second RNA molecule encoding a SARS-CoV-2 S protein comprising one or more mutations that are characteristic of an Omicron BA.4 or BA.5 variant is 1:3.

In some embodiments, a nucleic acid containing particle comprises a first RNA molecule encoding a SARS-CoV-2 S protein from a BA.3 Omicron variant and a second RNA molecule encoding a SARS-CoV-2 S protein comprising one or more mutations that are characteristic of a BA.4 or BA.5 Omicron variant. In some embodiments, the ratio of the first RNA molecule encoding a SARS-CoV-2 S protein comprising one or more mutations characteristic of a BA.3 Omicron variant strain and the second RNA molecule encoding a SARS-CoV-2 S protein comprising one or more mutations that are characteristic of an Omicron BA.4 or BA.5 variant is 1:1. In some embodiments, the ratio of the first RNA molecule encoding a SARS-CoV-2 S protein comprising one or more mutations characteristic of a BA.3 Omicron variant strain and the second RNA molecule encoding a SARS-CoV-2 S protein comprising one or more mutations that are characteristic of an Omicron BA.4 or BA.5 variant is 1:2. In some embodiments, the ratio of the first RNA molecule encoding a SARS-CoV-2 S protein comprising one or more mutations characteristic of a BA.3 Omicron variant strain and the second RNA molecule encoding a SARS-CoV-2 S protein comprising one or more mutations that are characteristic of an Omicron BA.4 or BA.5 variant is 1:3.

In some embodiments, a nucleic acid containing particle (e.g., in some embodiments an LNP as described herein) comprises three or more RNA molecules, each encoding a SARS-CoV-2 S protein comprising mutations of a different SARS-CoV-2 variant. In some embodiments, a nucleic acid containing particle comprises a first RNA molecule encoding a SARS-CoV-2 S protein from a Wuhan strain, a second RNA molecule encoding a SARS-CoV-2 S protein comprising one or more mutations that are characteristic of an Omicron variant (e.g., a BA.1, BA.2, BA.3, BA.4, or BA.5 Omicron variant), and a third RNA molecule encoding a SARS-CoV-2 S protein comprising one or more mutations that are characteristic of an Omicron variant (e.g., a BA.1, BA.2, BA.3, BA.4, or BA.5 Omicron variant), wherein the second and third RNA molecules encode a SARS-CoV-2 S protein comprising one or mutations characteristic of different Omicron subvariants. In some embodiments, a nucleic acid containing particle comprises a first RNA molecule encoding a SARS-CoV-2 S protein from a Wuhan strain, a second RNA molecule encoding a SARS-CoV-2 S protein comprising one or more mutations that are characteristic of a BA.1 Omicron variant, and a third RNA molecule encoding a SARS-CoV-2 S protein comprising one or more mutations that are characteristic of a BA.2 Omicron variant. In some embodiments, a nucleic acid containing particle comprises a first RNA molecule encoding a SARS-CoV-2 S protein from a Wuhan strain, a second RNA molecule encoding a SARS-CoV-2 S protein comprising one or more mutations that are characteristic of a BA.1 Omicron variant, and a third RNA molecule encoding a SARS-CoV-2 S protein comprising one or more mutations that are characteristic of a BA.2 Omicron variant. In some embodiments, a nucleic acid containing particle comprises a first RNA molecule encoding a SARS-CoV-2 S protein from a Wuhan strain, a second RNA molecule encoding a SARS-CoV-2 S protein comprising one or more mutations that are characteristic of a BA.1 Omicron variant, and a third RNA molecule encoding a SARS-CoV-2 S protein comprising one or more mutations that are characteristic of a BA.4/5 Omicron variant. In some embodiments, a nucleic acid containing particle comprises a first RNA molecule encoding a SARS-CoV-2 S protein from a Wuhan strain, a second RNA molecule encoding a SARS-CoV-2 S protein comprising one or more mutations that are characteristic of a BA.2 Omicron variant, and a third RNA molecule encoding a SARS-CoV-2 S protein comprising one or more mutations that are characteristic of a BA.4/5 Omicron variant. In some embodiments, a nucleic acid containing particle comprises a first RNA molecule encoding a SARS-CoV-2 S protein comprising one or more mutations that are characteristic of a BA.1 Omicron variant, a second RNA molecule encoding a SARS-CoV-2 S protein comprising one or more mutations that are characteristic of a BA.2 Omicron variant, and a third RNA molecule encoding a SARS-CoV-2 S protein comprising one or more mutations that are characteristic of a BA.4/5 Omicron variant.

In some embodiments, a nucleic acid containing particle comprises two or more RNA molecules, each comprising a nucleotide sequence encoding an antigen (e.g., an HA protein) associated with a different influenza virus. In some embodiments, a nucleic acid containing particle comprises three or more RNA molecules, each comprising a nucleotide sequence encoding an antigen (e.g., an HA protein) associated with a different influenza virus. In some embodiments, a nucleic acid containing particle comprises four or more RNA molecules, each comprising a nucleotide sequence encoding an antigen (e.g., an HA protein) associated with a different influenza virus. In some embodiments, a nucleic acid containing particle comprises an RNA molecule comprising a nucleotide sequence encoding an antigenic polypeptide associated with an H1N1 influenza virus, an RNA molecule comprising a nucleotide sequence encoding an antigenic polypeptide associated with an H3N2 influenza virus, an RNA molecule comprising a nucleotide sequence encoding an antigenic polypeptide associated with a B/Victoria lineage influenza virus, and an RNA molecule comprising a nucleotide sequence encoding an antigenic polypeptide associated with a B/Yamagata influenza virus. In some embodiments, each RNA in a composition comprising a nucleotide sequence encoding an antigenic polypeptide associated with an influenza virus is formulated in the same nucleic acid containing particle. In some embodiments, each RNA in a composition comprising a nucleotide sequence encoding an antigenic polypeptide associated with an influenza virus is formulated in separate nucleic acid containing particles.

In some embodiments, a nucleic acid particle comprises (i) two or more RNAs, each comprising a nucleotide sequence encoding an antigenic polypeptide associated with a different coronavirus, and (i) two or more RNAS, each comprising a nucleotide sequence encoding an antigenic polypeptide associated with a different influenza virus.

In some embodiments, a nucleic acid particle comprises (i) two or more RNAS, each comprising a nucleotide sequence encoding an antigenic polypeptide associated with a different coronavirus, and (i) four or more RNAs, each comprising a nucleotide sequence encoding an antigenic polypeptide associated with a different influenza virus.

In some embodiments, RNAs comprising an antigenic polypeptide associated with a certain infectious agent are formulated in the same nucleic acid particle. For example, in some embodiments, a composition comprises (i) a first nucleic acid particle comprising two or more RNAs, each comprising a nucleotide sequence encoding an antigenic polypeptide associated with a different coronavirus, and (ii) a second nucleic acid particle comprising four or more RNAs, each comprising a nucleotide sequence encoding an antigenic polypeptide associated with a different influenza virus (e.g., an RNA comprising a nucleotide sequence encoding an HA protein associated with an H1N1 influenza virus, an RNA comprising a nucleotide sequence encoding an HA protein associated with an H3N2 influenza virus, an RNA comprising a nucleotide sequence encoding an HA protein associated with an B/Victoria lineage influenza virus, and an RNA comprising a nucleotide sequence encoding an HA protein associated with an B/Yamagata influenza virus).

In some embodiments, a nucleic acid containing particle (e.g., in some embodiments an LNP as described herein) comprising two or more RNA molecules, comprises each RNA molecule in the same amount (i.e., at a 1:1 ratio).

In some embodiments, a nucleic acid containing particle (e.g., in some embodiments an LNP as described herein) comprising two or more RNA molecules, comprises a different amount of each RNA molecule. For example, in some embodiments, a nucleic acid containing particle comprises a first RNA molecule and a second RNA molecule, where the first RNA molecule is present in an amount that is 0.01 to 100 times that of the second RNA molecule (e.g., wherein the amount of the first RNA molecule is 0.01 to 50, 0.01 to 4, 0.01 to 30, 0.01 to 25, 0.01 to 20, 0.01 to 15, 0.01 to 10, 0.01 to 9, 0.01 to 8, 0.01 to 7, 0.01 to 6, 0.01 to 5, 0.01 to 4, 0.01 to 3, 0.01 to 2, 0.01 to 1.5, 1 to 50, 1 to 4, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1 to 1.5 times higher than the second RNA molecule). In some embodiments, a nucleic acid containing particle comprises a first RNA molecule and a second RNA molecule, wherein the concentration of the first RNA molecule is 1 to 10 times that of the second RNA molecule. In some embodiments, a nucleic acid containing particle comprises a first RNA molecule and a second RNA molecule, wherein the concentration of the first RNA molecule is 1 to 5 times that of the second RNA molecule. In some embodiments, a nucleic acid containing particle comprises a first RNA molecule and a second RNA molecule, wherein the concentration of the first RNA molecule is 1 to 3 times that of the second RNA molecule. In some embodiments, a nucleic acid containing particle comprises a first RNA molecule and a second RNA molecule, wherein the concentration of the first RNA molecule is 2 times that of the second RNA molecule. In some embodiments, a nucleic acid containing particle comprises a first RNA molecule and a second RNA molecule, wherein the concentration of the first RNA molecule is 3 times that of the second RNA molecule.

In some embodiments, a nucleic acid containing particle (e.g., in some embodiments an LNP as described herein) comprising three RNA molecules, comprises each RNA molecule in the same amount (i.e., at a 1:1:1 ratio).

In some embodiments, a nucleic acid containing particle (e.g., in some embodiments an LNP as described herein) comprising three RNA molecules, comprises a different amount of each RNA molecule. For example, in some embodiments, the ratio of first RNA molecule:second RNA molecule:third RNA molecule is 1:0.01-100:0.01-100 (e.g., 1:0.01-50:0.01-50; 1:0.01-40:0.01-40; 1:0.01-30:0.01-25; 1:0.01-25:0.01-25; 1:0.01-20:0.01-20; 1:0.01-15:0.01-15; 1:0.01-10:0.01-9; 1:0.01-9:0.01-9; 1:0.01-8:0.01-8; 1:0.01-7:0.01-7; 1:0.01-6:0.01-6; 1:0.01-5:0.01-5; 1:0.01-4:0.01-4; 1:0.01-3:0.01-3; 1:0.01-2:0.01-2; or 1:0.01-1.5:0.01-1.5). In some embodiments, the ratio of first RNA molecule:second RNA molecule:third RNA molecule is 1:1:3. In some embodiments, the ratio of first RNA molecule:second RNA molecule:third RNA molecule is 1:3:3.

In some embodiments, a first RNA molecule encoding a SARS-CoV-2 S protein from a Wuhan strain comprises a nucleotide sequence that encodes the amino acid sequence of SEQ ID NO: 7. In some embodiments, a first RNA molecule encoding a SARS-CoV-2 S protein from a Wuhan strain comprises a nucleotide sequence that is at least 80% identical (e.g., at least 85%, at least 90%, at least 91%, at least 92%, at least 93%, at least 94%, at least 95%, at least 96%, at least 97%, at least 98%, or at least 99% identical) to SEQ ID NO: 9. In some embodiments, a first RNA molecule encoding a SARS-CoV-2 S protein from a Wuhan strain comprises a nucleotide sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 20. In some embodiments, a first RNA molecule encoding a SARS-CoV-2 S protein from a Wuhan strain comprises a nucleotide sequence that encodes an amino acid sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 7. In some embodiments, a second RNA molecule encoding a SARS-CoV-2 S protein having one or more mutations that are characteristic of an Omicron variant comprises a nucleotide sequence that encodes the amino acid sequence of SEQ ID NO: 49. In some embodiments, a second RNA molecule encoding a SARS-CoV-2 S protein comprising one or more mutations characteristic of an Omicron variant comprises a nucleotide sequence that is at least 80% identical (e.g., at least 85%, at least 90%, at least 91%, at least 92%, at least 93%, at least 94%, at least 95%, at least 96%, at least 97%, at least 98%, or at least 99% identical) to SEQ ID NO: 50. In some embodiments, a second RNA molecule encoding a SARS-CoV-2 S protein comprising one or more mutations characteristic of an Omicron variant comprises a nucleotide sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 51. In some embodiments, a second RNA molecule encoding a SARS-CoV-2 S protein comprising one or more mutations characteristic of an Omicron variant comprises a nucleotide sequence that encodes an amino acid sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 49.

In some embodiments, a second RNA molecule encoding a SARS-CoV-2 S protein having one or more mutations that are characteristic of an Omicron variant comprises a nucleotide sequence that encodes the amino acid sequence of SEQ ID NO: 64. In some embodiments, a second RNA molecule encoding a SARS-CoV-2 S protein comprising one or more mutations characteristic of an Omicron variant comprises a nucleotide sequence that is at least 80% identical (e.g., at least 85%, at least 90%, at least 91%, at least 92%, at least 93%, at least 94%, at least 95%, at least 96%, at least 97%, at least 98%, or at least 99% identical) to SEQ ID NO: 65. In some embodiments, a second RNA molecule encoding a SARS-CoV-2 S protein comprising one or more mutations characteristic of an Omicron variant comprises a nucleotide sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 67. In some embodiments, a second RNA molecule encoding a SARS-CoV-2 S protein comprising one or more mutations characteristic of an Omicron variant comprises a nucleotide sequence that encodes an amino acid sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 64.

In some embodiments, a second RNA molecule encoding a SARS-CoV-2 S protein having one or more mutations that are characteristic of an Omicron variant comprises a nucleotide sequence that encodes the amino acid sequence of SEQ ID NO: 69. In some embodiments, a second RNA molecule encoding a SARS-CoV-2 S protein comprising one or more mutations characteristic of an Omicron variant comprises a nucleotide sequence that is at least 80% identical (e.g., at least 85%, at least 90%, at least 91%, at least 92%, at least 93%, at least 94%, at least 95%, at least 96%, at least 97%, at least 98%, or at least 99% identical) to SEQ ID NO: 70. In some embodiments, a second RNA molecule encoding a SARS-CoV-2 S protein comprising one or more mutations characteristic of an Omicron variant comprises a nucleotide sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 72. In some embodiments, a second RNA molecule encoding a SARS-CoV-2 S protein comprising one or more mutations characteristic of an Omicron variant comprises a nucleotide sequence that encodes an amino acid sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 69.

In some embodiments, a second RNA molecule encoding a SARS-CoV-2 S protein having one or more mutations that are characteristic of an Omicron variant comprises a nucleotide sequence that encodes the amino acid sequence of SEQ ID NO: 74. In some embodiments, a second RNA molecule encoding a SARS-CoV-2 S protein comprising one or more mutations characteristic of an Omicron variant comprises a nucleotide sequence that is at least 80% identical (e.g., at least 85%, at least 90%, at least 91%, at least 92%, at least 93%, at least 94%, at least 95%, at least 96%, at least 97%, at least 98%, or at least 99% identical) to SEQ ID NO: 75. In some embodiments, a second RNA molecule encoding a SARS-CoV-2 S protein comprising one or more mutations characteristic of an Omicron variant comprises a nucleotide sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 77. In some embodiments, a second RNA molecule encoding a SARS-CoV-2 S protein comprising one or more mutations characteristic of an Omicron variant comprises a nucleotide sequence that encodes an amino acid sequence that is at least 80% identical to (e.g., at least 85%, at least 90%, at least 91%, at least 92%, at least 93%, at least 94%, at least 95%, at least 96%, at least 97%, at least 98%, or at least 99% identical to) SEQ ID NO: 74.

In some embodiments, a nucleic acid containing particle (e.g., in some embodiments an LNP as described herein) comprises: a first RNA molecule comprising a nucleotide sequence that encodes the amino acid sequence of SEQ ID NO: 7 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 7); and a second RNA molecule comprising a nucleotide sequence that encodes the amino acid sequence of SEQ ID NO: 49 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 49. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 49 or a sequence that is at least 80% identical to SEQ ID NO: 49 is 1:1. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 49 or a sequence that is at least 80% identical to SEQ ID NO: 49 is 1:2. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 49 or a sequence that is at least 80% identical to SEQ ID NO: 49 is 1:3.

In some embodiments, a nucleic acid containing particle (e.g., in some embodiments an LNP as described herein) comprises: a first RNA molecule comprising a nucleotide sequence of SEQ ID NO: 9 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 9); and a second RNA molecule comprising a nucleotide sequence of SEQ ID NO: 50 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 50. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 50 or a sequence that is at least 80% identical to SEQ ID NO: 50 is 1:1. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 50 or a sequence that is at least 80% identical to SEQ ID NO: 50 is 1:2. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 50 or a sequence that is at least 80% identical to SEQ ID NO: 50 is 1:3.

In some embodiments, a nucleic acid containing particle (e.g., in some embodiments an LNP as described herein) comprises: a first RNA molecule comprising a nucleotide sequence of SEQ ID NO: 20 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 20; and a second RNA molecule comprising a nucleotide sequence of SEQ ID NO: 51 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 51. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 51 or a sequence that is at least 80% identical to SEQ ID NO: 51 is 1:1. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 51 or a sequence that is at least 80% identical to SEQ ID NO: 51 is 1:2. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 51 or a sequence that is at least 80% identical to SEQ ID NO: 51 is 1:3.

In some embodiments, a nucleic acid containing particle (e.g., in some embodiments an LNP as described herein) comprises: a first RNA molecule comprising a nucleotide sequence that encodes the amino acid sequence of SEQ ID NO: 7 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 7); and a second RNA molecule comprising a nucleotide sequence that encodes the amino acid sequence of SEQ ID NO: 64 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 64. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 49 or a sequence that is at least 80% identical to SEQ ID NO: 64 is 1:1. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 64 or a sequence that is at least 80% identical to SEQ ID NO: 64 is 1:2. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 64 or a sequence that is at least 80% identical to SEQ ID NO: 64 is 1:3.

In some embodiments, a nucleic acid containing particle (e.g., in some embodiments an LNP as described herein) comprises: a first RNA molecule comprising a nucleotide sequence of SEQ ID NO: 9 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 9); and a second RNA molecule comprising a nucleotide sequence of SEQ ID NO: 65 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 65. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 65 or a sequence that is at least 80% identical to SEQ ID NO: 65 is 1:1. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 65 or a sequence that is at least 80% identical to SEQ ID NO: 65 is 1:2. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 65 or a sequence that is at least 80% identical to SEQ ID NO: 65 is 1:3.

In some embodiments, a nucleic acid containing particle (e.g., in some embodiments an LNP as described herein) comprises: a first RNA molecule comprising a nucleotide sequence of SEQ ID NO: 20 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 20; and a second RNA molecule comprising a nucleotide sequence of SEQ ID NO: 51 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 67. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 67 or a sequence that is at least 80% identical to SEQ ID NO: 67 is 1:1. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 67 or a sequence that is at least 80% identical to SEQ ID NO: 67 is 1:2. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 67 or a sequence that is at least 80% identical to SEQ ID NO: 67 is 1:3.

In some embodiments, a nucleic acid containing particle (e.g., in some embodiments an LNP as described herein) comprises: a first RNA molecule comprising a nucleotide sequence that encodes the amino acid sequence of SEQ ID NO: 7 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 7); and a second RNA molecule comprising a nucleotide sequence that encodes the amino acid sequence of SEQ ID NO: 69 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 69. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 69 or a sequence that is at least 80% identical to SEQ ID NO: 69 is 1:1. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 69 or a sequence that is at least 80% identical to SEQ ID NO: 69 is 1:2. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 69 or a sequence that is at least 80% identical to SEQ ID NO: 69 is 1:3.

In some embodiments, a nucleic acid containing particle (e.g., in some embodiments an LNP as described herein) comprises: a first RNA molecule comprising a nucleotide sequence of SEQ ID NO: 9 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 9); and a second RNA molecule comprising a nucleotide sequence of SEQ ID NO: 70 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 70. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 70 or a sequence that is at least 80% identical to SEQ ID NO: 70 is 1:1. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 70 or a sequence that is at least 80% identical to SEQ ID NO: 70 is 1:2. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 50 or a sequence that is at least 80% identical to SEQ ID NO: 70 is 1:3.

In some embodiments, a nucleic acid containing particle (e.g., in some embodiments an LNP as described herein) comprises: a first RNA molecule comprising a nucleotide sequence of SEQ ID NO: 20 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 20; and a second RNA molecule comprising a nucleotide sequence of SEQ ID NO: 72 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 72. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 72 or a sequence that is at least 80% identical to SEQ ID NO: 72 is 1:1. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 72 or a sequence that is at least 80% identical to SEQ ID NO: 72 is 1:2. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 72 or a sequence that is at least 80% identical to SEQ ID NO: 72 is 1:3.

In some embodiments, a nucleic acid containing particle (e.g., in some embodiments an LNP as described herein) comprises: a first RNA molecule comprising a nucleotide sequence that encodes the amino acid sequence of SEQ ID NO: 7 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 7); and a second RNA molecule comprising a nucleotide sequence that encodes the amino acid sequence of SEQ ID NO: 74 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 74. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 74 or a sequence that is at least 80% identical to SEQ ID NO: 74 is 1:1. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 74 or a sequence that is at least 80% identical to SEQ ID NO: 74 is 1:2. In some embodiments, the ratio of the first RNA molecule that encodes the amino acid sequence of SEQ ID NO: 7 or a sequence that is at least 80% identical to SEQ ID NO: 7 to the second RNA molecule that encodes the amino acid sequence of SEQ ID NO: 74 or a sequence that is at least 80% identical to SEQ ID NO: 74 is 1:3.

In some embodiments, a nucleic acid containing particle (e.g., in some embodiments an LNP as described herein) comprises: a first RNA molecule comprising a nucleotide sequence of SEQ ID NO: 9 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 9); and a second RNA molecule comprising a nucleotide sequence of SEQ ID NO: 75 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 75. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 75 or a sequence that is at least 80% identical to SEQ ID NO: 75 is 1:1. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 75 or a sequence that is at least 80% identical to SEQ ID NO: 75 is 1:2. In some embodiments, the ratio of the first RNA molecule comprising the nucleotide sequence of SEQ ID NO: 9 or a sequence that is at least 80% identical to SEQ ID NO: 9 to the second RNA molecule that comprises a nucleotide sequence of SEQ ID NO: 75 or a sequence that is at least 80% identical to SEQ ID NO: 75 is 1:3.

In some embodiments, a nucleic acid containing particle (e.g., in some embodiments an LNP as described herein) comprises: a first RNA molecule comprising a nucleotide sequence of SEQ ID NO: 20 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 20; and a second RNA molecule comprising a nucleotide sequence of SEQ ID NO: 77 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 77. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 77 or a sequence that is at least 80% identical to SEQ ID NO: 77 is 1:1. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 77 or a sequence that is at least 80% identical to SEQ ID NO: 77 is 1:2. In some embodiments, the ratio of the first RNA molecule comprising the nucleotide sequence of SEQ ID NO: 20 or a sequence that is at least 80% identical to SEQ ID NO: 20 to the second RNA molecule that comprises a nucleotide sequence of SEQ ID NO: 77 or a sequence that is at least 80% identical to SEQ ID NO: 77 is 1:3.

In some embodiments, a nucleic acid containing particle (e.g., in some embodiments an LNP as described herein) comprises: a first RNA molecule comprising a nucleotide sequence that encodes the amino acid sequence of SEQ ID NO: 7 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 7); and a second RNA molecule comprising a nucleotide sequence that encodes the amino acid sequence of SEQ ID NO: 55, 58, or 61 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 55, 58, or 61.

In some embodiments, a nucleic acid containing particle (e.g., in some embodiments an LNP as described herein) comprises: a first RNA molecule comprising a nucleotide sequence of SEQ ID NO: 9 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 9; and a second RNA molecule comprising a nucleotide sequence of SEQ ID NO: 56, 59, or 62 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 56, 59, or 62.

In some embodiments, a nucleic acid containing particle (e.g., in some embodiments an LNP as described herein) comprises: a first RNA molecule comprising a nucleotide sequence of SEQ ID NO: 20 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 20; and a second RNA molecule comprising a nucleotide sequence of SEQ ID NO: 57, 60, or 63 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 57, 60, or 63.

In some embodiments, a nucleic acid containing particle (e.g., in some embodiments an LNP as described herein) comprises: a first RNA molecule comprising a nucleotide sequence that encodes the amino acid sequence of SEQ ID NO: 58 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 58; and a second RNA molecule comprising a nucleotide sequence that encodes an amino acid sequence of SEQ ID NO: 49, 55, or 61 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 49, 55, or 61.

In some embodiments, a nucleic acid containing particle (e.g., in some embodiments an LNP as described herein) comprises: a first RNA molecule comprising a nucleotide sequence of SEQ ID NO: 59 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 59; and a second RNA molecule comprising a nucleotide sequence of SEQ ID NO: 50, 56, or 62, or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 50, 56, or 62.

In some embodiments, a nucleic acid containing particle (e.g., in some embodiments an LNP as described herein) comprises: a first RNA molecule comprising a nucleotide sequence of SEQ ID NO: 60 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 60; and a second RNA molecule comprising a nucleotide sequence of SEQ ID NO: 51, 57, or 63, or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 51, 57, or 63.

In some embodiments, a nucleic acid containing particle (e.g., in some embodiments an LNP as described herein) comprises: a first RNA molecule comprising a nucleotide sequence that encodes the amino acid sequence of SEQ ID NO: 49 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 49; and a second RNA molecule comprising a nucleotide sequence that encodes the amino acid sequence of SEQ ID NO: 55 or 61 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 55 or 61.

In some embodiments, a nucleic acid containing particle (e.g., in some embodiments an LNP as described herein) comprises: a first RNA molecule comprising a nucleotide sequence of SEQ ID NO: 50 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 50; and a second RNA molecule comprising a nucleotide sequence of SEQ ID NO: 56 or 62 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 56 or 62.

In some embodiments, a nucleic acid containing particle (e.g., in some embodiments an LNP as described herein) comprises: a first RNA molecule comprising a nucleotide sequence of SEQ ID NO: 51 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 51; and a second RNA molecule comprising a nucleotide sequence of SEQ ID NO: 57 or 63 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 57 or 63.

In some embodiments, a nucleic acid containing particle (e.g., in some embodiments an LNP as described herein) comprises: a first RNA molecule comprising a nucleotide sequence that encodes the amino acid sequence of SEQ ID NO: 55 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 55; and a second RNA molecule comprising a nucleotide sequence that encodes the amino acid sequence of SEQ ID NO: 61 or an amino acid sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 61.

In some embodiments, a nucleic acid containing particle (e.g., in some embodiments an LNP as described herein) comprises: a first RNA molecule comprising a nucleotide sequence of SEQ ID NO: 56 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 56; and a second RNA molecule comprising a nucleotide sequence of SEQ ID NO: 62, or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 62.

In some embodiments, a nucleic acid containing particle (e.g., in some embodiments an LNP as described herein) comprises: a first RNA molecule comprising a nucleotide sequence of SEQ ID NO: 57 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 57; and a second RNA molecule comprising a nucleotide sequence of SEQ ID NO: 63 or a nucleotide sequence that is at least 80% (e.g., at least 85%, at least 90%, at least 91%, at least 92%, at least 93%, at least 94%, at least 95%, at least 96%, at least 97%, at least 98%, at least 99% or higher) identical to SEQ ID NO: 63.

In some embodiments, a particle (e.g., in some embodiments an LNP) containing nucleic acids (e.g., RNAs) encoding different polypeptides can be formed by mixing a plurality of (e.g., at least two, at least three, or more) RNA molecules with particle-forming components (e.g., lipids). In some embodiments, nucleic acids (e.g., RNAs) encoding different polypeptides can be mixed (e.g., in some embodiments in substantially equal proportions, e.g., in some embodiments at a 1:1 ratio when two RNA molecules are present) prior to mixing with particle-forming components (e.g., lipids).

In some embodiments, two or more RNA molecules each encoding a different polypeptide (e.g., as described herein) can be mixed with particle-forming agents to form nucleic acid containing particles as described above. In alternative embodiments, two or more RNA molecules each encoding a different polypeptide (e.g., as described herein) can be formulated into separate particle compositions, which are then mixed together. For example, in some embodiments, individual populations of nucleic acid containing particles, each population comprising an RNA molecule encoding a different immunogenic polypeptide or immunogenic fragment thereof (e.g., as described herein), can be separately formed and then mixed together, for example, prior to filling into vials during a manufacturing process, or immediately prior to administration (e.g., by an administering health-care professional)).

Accordingly, in some embodiments, described herein is a composition comprises two or more populations of particles (e.g., in some embodiments, lipid nanoparticles), each population comprising at least one RNA molecule encoding a different immunogenic polypeptide or immunogenic fragment thereof (e.g., a SARS-CoV-2 S protein, or fragments thereof, from a different variant). In some embodiments, each population may be provided in a composition at a desirable proportion (e.g., in some embodiments, each population may be provided in a composition in an amount that provides the same amount of RNA molecules).

### Cationic Polymer

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneamine, in particular protamine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the present disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneamine comprises polyethylenimine and/or polypropylenimine, preferably polyethyleneimine. A preferred polyalkyleneamine is polyethyleneimine (PEI). The average molecular weight of PEI is preferably 0.75.102 to 107 Da, preferably 1000 to 105 Da, more preferably 10000 to 40000 Da, more preferably 15000 to 30000 Da, even more preferably 20000 to 25000 Da.

Preferred according to the present disclosure is linear polyalkyleneamine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipid and Lipid-Like Material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the present disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol. Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the present disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids or Lipid-Like Materials

The nucleic acid particles described herein may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological PH.

For purposes of the present disclosure, such “cationically ionizable” lipids or lipid-like materials are comprised by the term “cationic lipid or lipid-like material” unless contradicted by the circumstances.

In one embodiment, the cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl) cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2 (dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

### Additional Lipids or Lipid-Like Materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the nucleic acid particles include both a cationic lipid and an additional lipid.

In one embodiment, particles described herein include a polymer conjugated lipid such as a pegylated lipid. The term “pegylated lipid” refers to a molecule comprising both a lipid portion and a polyethylene glycol portion. Pegylated lipids are known in the art.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

### Lipoplex Particles

In certain embodiments of the present disclosure, RNA described herein may be present in RNA lipoplex particles.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE.

In one embodiment, a RNA lipoplex particle is a nanoparticle.

In certain embodiments, RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, RNA lipoplex particles have an average diameter that ranges from about 250 nm to about 700 nm.

In another embodiment, RNA lipoplex particles have an average diameter that ranges from about 300 nm to about 500 nm. In an exemplary embodiment, RNA lipoplex particles have an average diameter of about 400 nm.

RNA lipoplex particles and compositions comprising RNA lipoplex particles described herein are useful for delivery of RNA to a target tissue after parenteral administration, in particular after intravenous administration. RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of RNA lipoplex particles, RNA accumulation and/or RNA expression in the spleen occurs. Thus, RNA lipoplex particles of the present disclosure may be used for expressing RNA in the spleen. In an embodiment, after administration of RNA lipoplex particles, no or essentially no RNA accumulation and/or RNA expression in the lung and/or liver occurs. In one embodiment, after administration of RNA lipoplex particles, RNA accumulation and/or RNA expression in antigen presenting cells, such as professional antigen presenting cells in the spleen occurs. Thus, RNA lipoplex particles of the present disclosure may be used for expressing RNA in such antigen presenting cells. In one embodiment, the antigen presenting cells are dendritic cells and/or macrophages.

### Lipid Nanoparticles (LNPs)

In one embodiment, nucleic acid such as RNA described herein is administered in the form of lipid nanoparticles (LNPs). LNPs may comprise any lipid capable of forming a particle to which the one or more nucleic acid molecules are attached, or in which the one or more nucleic acid molecules are encapsulated.

In one embodiment, an LNP comprises one or more cationic lipids, and one or more stabilizing lipids. Stabilizing lipids include neutral lipids and pegylated lipids.

In one embodiment, an LNP comprises a cationic lipid, a neutral lipid, a steroid, a polymer conjugated lipid; and RNA, encapsulated within or associated with the lipid nanoparticle.

In one embodiment, an LNP comprises from 20 to 60 mol percent, 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationic lipid. In one embodiment, the LNP comprises about 47.0, 47.1, 47.2, 47.3, 47.4, 47.5, 47.6, 47.7, 47.8, 47.9 or 48.0 mol percent of the cationic lipid.

In one embodiment, the neutral lipid is present in a concentration ranging from 5 to 25 mol percent, 5 to 15 mol percent, from 7 to 13 mol percent, or from 9 to 11 mol percent. In one embodiment, the neutral lipid is present in a concentration of about 9.5, 10 or 10.5 mol percent.

In one embodiment, the steroid is present in a concentration ranging from 25 to 55 mol percent, 30 to 50 mol percent, from 35 to 45 mol percent or from 38 to 43 mol percent. In one embodiment, the steroid is present in a concentration of about 40, 41, 42, 43, 44, 45 or 46 mol percent.

In one embodiment, an LNP comprises from 0.5 to 15 mol percent, 1 to 10 mol percent, from 1 to 5 mol percent, or from 1 to 2.5 mol percent of the polymer conjugated lipid.

In one embodiment, an LNP comprises from 20 to 60 mol percent cationic or lipid, 5 to 25 mol percent non-cationic lipid (e.g., neutral lipid), 25 to 55 mol percent sterol or steroid, and 0.5 to 15 mol percent polymer-conjugated lipid (e.g., PEG-modified lipid).

In one embodiment, an LNP comprises from 40 to 50 mol percent a cationic lipid; from 5 to 15 mol percent of a neutral lipid; from 35 to 45 mol percent of a steroid; from 1 to 10 mol percent of a polymer conjugated lipid; and RNA, encapsulated within or associated with the lipid nanoparticle.

In one embodiment, the mol percent is determined based on total mol of lipid present in the lipid nanoparticle.

In one embodiment, the neutral lipid is selected from the group consisting of DSPC, DPPC, DMPC, DOPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

In one embodiment, the steroid is cholesterol.

In one embodiment, the polymer conjugated lipid is a pegylated lipid. In one embodiment, the pegylated lipid has the following structure:

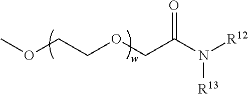

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In one embodiment, R¹² and R¹³ are each independently straight, saturated alkyl chains containing from 12 to 16 carbon atoms. In one embodiment, w has a mean value ranging from 40 to 55. In one embodiment, the average w is about 45. In one embodiment, R¹² and R¹³ are each independently a straight, saturated alkyl chain containing about 14 carbon atoms, and w has a mean value of about 45.

In one embodiment, the pegylated lipid is DMG-PEG 2000, e.g., having the following structure:

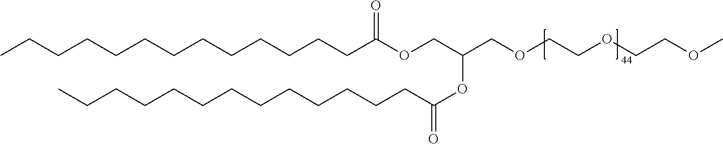

In some embodiments, the cationic lipid component of the LNPs has the structure of Formula (III):

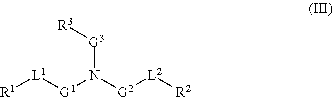

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR^{a}C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

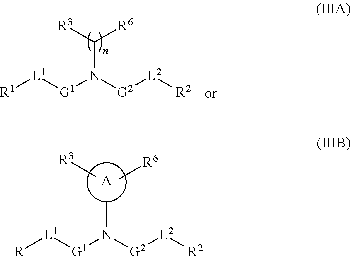

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

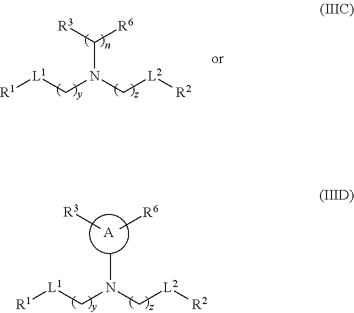

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is (C═O)O—. In some different embodiments of Formula (III), the lipid has one of the following structures (IIIE) or (IIIF):

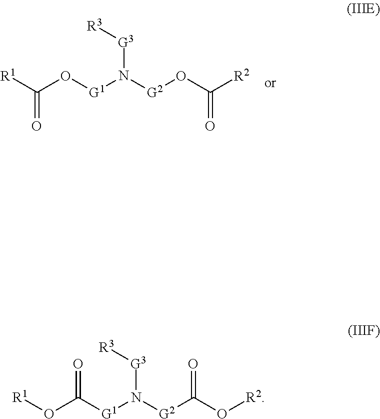

In some of the foregoing embodiments of Formula (III), the lipid has one of the following structures (IIIG), (IIIH), (IIII), or (IIIJ):

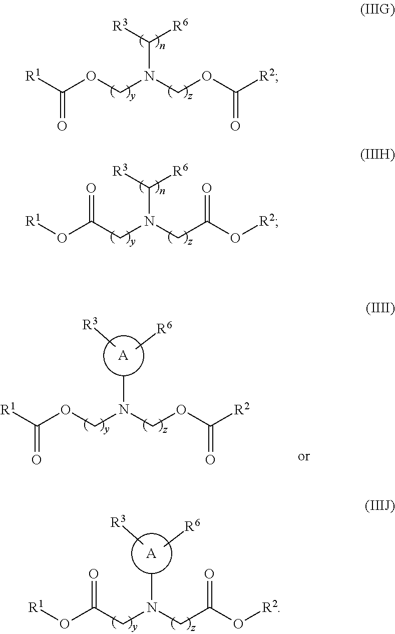

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

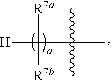

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈alkyl. For example, in some embodiments, C₁-C₈alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

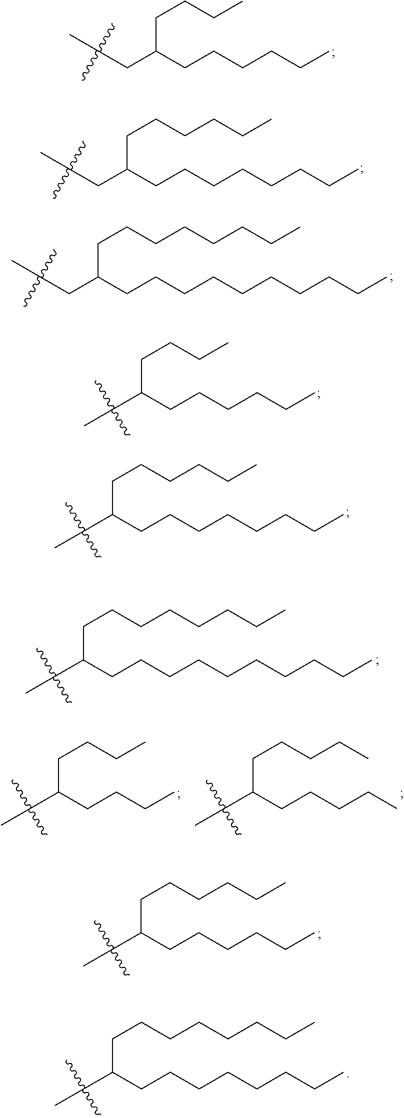

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in the table below.

TABLE 25
Representative Compounds of Formula (III).

| No. | Structure |
|---|---|
| III-1 | 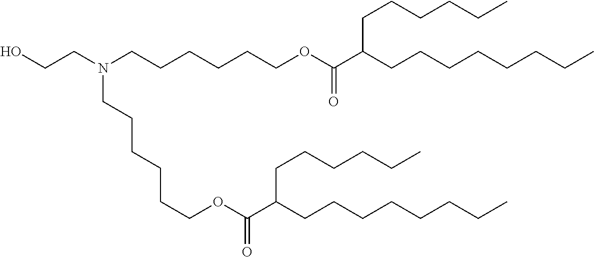 [SMILES: CCCCCCCCC(CCCCCC)C(=O)OCCCCCCN(CCO)CCCCCCOC(=O)C(CCCCCC)CCCCCCCC] |
| III-2 | 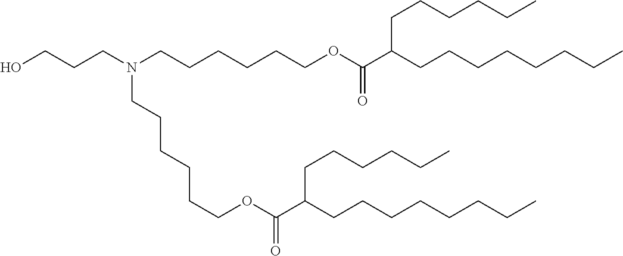 [SMILES: CCCCCCCCC(CCCCCC)C(=O)OCCCCCCN(CCCO)CCCCCCOC(=O)C(CCCCCC)CCCCCCCC] |
| III-3 | 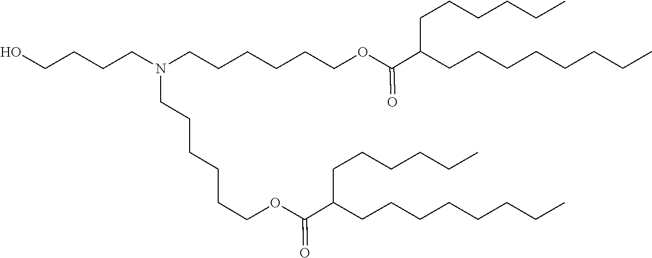 [SMILES: CCCCCCCCC(CCCCCC)C(=O)OCCCCCCN(CCCCO)CCCCCCOC(=O)C(CCCCCC)CCCCCCCC] |
| III-4 | 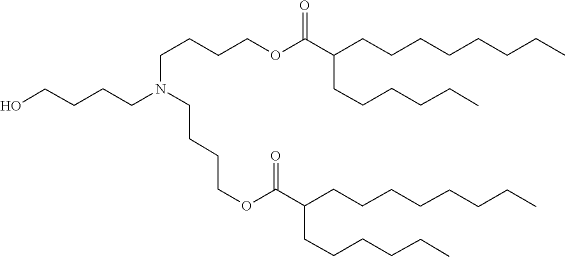 [SMILES: CCCCCCCCC(CCCCCC)C(=O)OCCCCN(CCCCO)CCCCOC(=O)C(CCCCCC)CCCCCCCC] |
| III-5 | 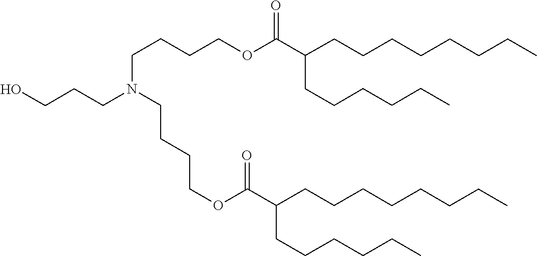 [SMILES: CCCCCCCCC(CCCCCC)C(=O)OCCCCN(CCCO)CCCCOC(=O)C(CCCCCC)CCCCCCCC] |
| III-6 | 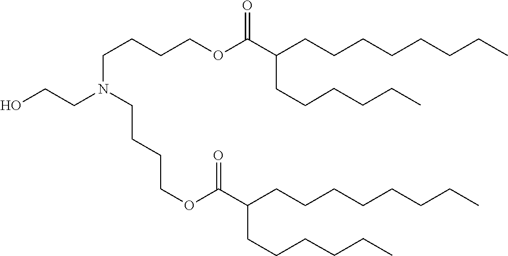 [SMILES: CCCCCCCCC(CCCCCC)C(=O)OCCCCN(CCO)CCCCOC(=O)C(CCCCCC)CCCCCCCC] |
| III-7 | 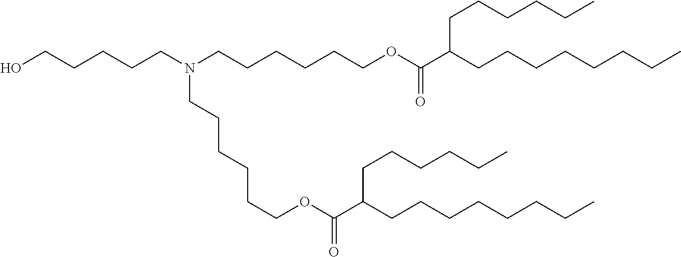 [SMILES: CCCCCCCCC(CCCCCC)C(=O)OCCCCCCN(CCCCCO)CCCCCCOC(=O)C(CCCCCC)CCCCCCCC] |
| III-8 | 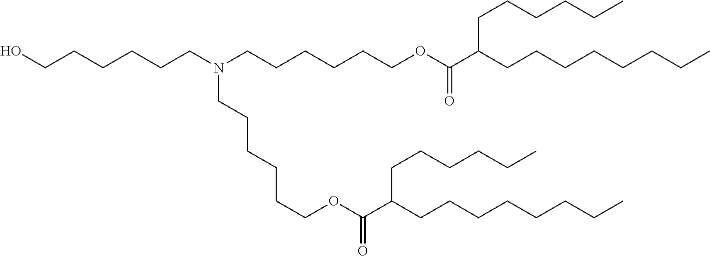 [SMILES: CCCCCCCCC(CCCCCC)C(=O)OCCCCCCN(CCCCCCO)CCCCCCOC(=O)C(CCCCCC)CCCCCCCC] |
| III-9 | 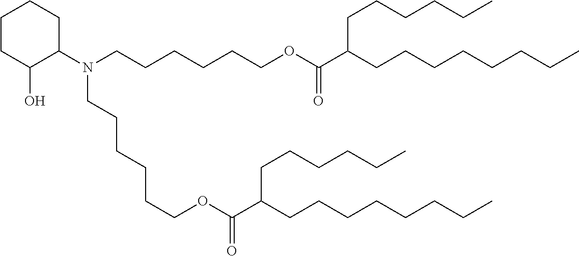 [SMILES: CCCCCCCCC(CCCCCC)C(=O)OCCCCCCN(CCCCCCOC(=O)C(CCCCCC)CCCCCCCC)C1CCCCC1O] |
| III-10 | 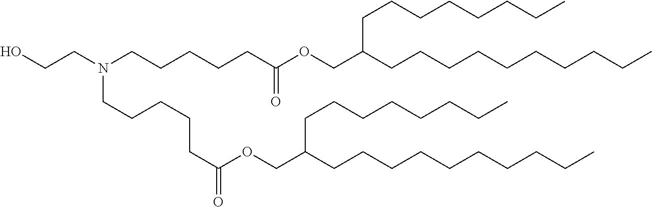 [SMILES: CCCCCCCCCCC(CCCCCCCC)COC(=O)CCCCCN(CCO)CCCCCC(=O)OCC(CCCCCCCC)CCCCCCCCCC] |
| III-11 | 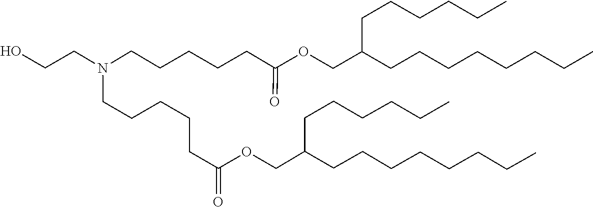 [SMILES: CCCCCCCCC(CCCCCC)COC(=O)CCCCCN(CCO)CCCCCC(=O)OCC(CCCCCC)CCCCCCCC] |
| III-12 | 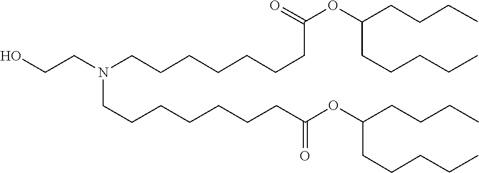 [SMILES: CCCCCC(CCCC)OC(=O)CCCCCCCN(CCO)CCCCCCCC(=O)OC(CCCC)CCCCC] |
| III-13 | 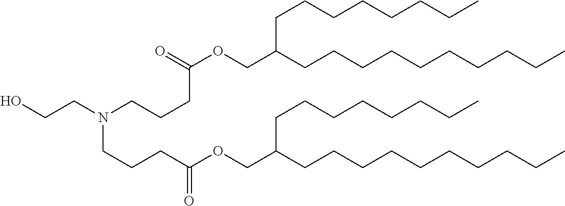 [SMILES: CCCCCCCCCCC(CCCCCCCC)COC(=O)CCCN(CCO)CCCC(=O)OCC(CCCCCCCC)CCCCCCCCCC] |
| III-14 | 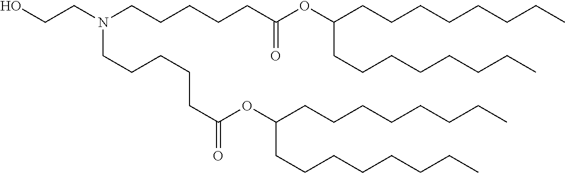 [SMILES: CCCCCCCCC(CCCCCCCC)OC(=O)CCCCCN(CCO)CCCCCC(=O)OC(CCCCCCCC)CCCCCCCC] |
| III-15 | 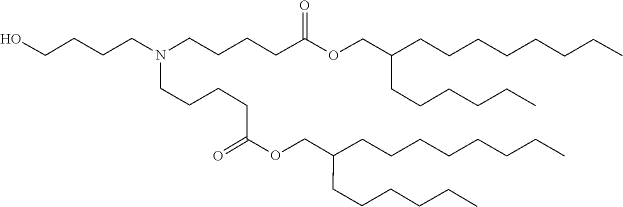 [SMILES: CCCCCCCCC(CCCCCC)COC(=O)CCCCN(CCCCO)CCCCC(=O)OCC(CCCCCC)CCCCCCCC] |
| III-16 | 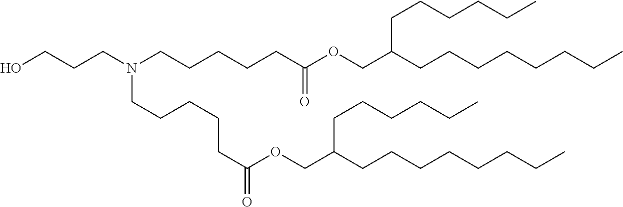 [SMILES: CCCCCCCCC(CCCCCC)COC(=O)CCCCCN(CCCO)CCCCCC(=O)OCC(CCCCCC)CCCCCCCC] |
| III-17 | 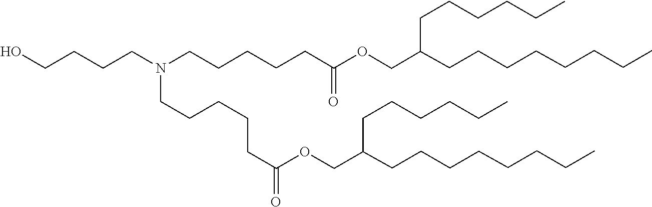 [SMILES: CCCCCCCCC(CCCCCC)COC(=O)CCCCCN(CCCCO)CCCCCC(=O)OCC(CCCCCC)CCCCCCCC] |
| III-18 | 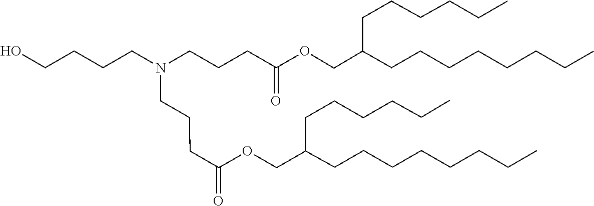 [SMILES: CCCCCCCCC(CCCCCC)COC(=O)CCCN(CCCCO)CCCC(=O)OCC(CCCCCC)CCCCCCCC] |
| III-19 | 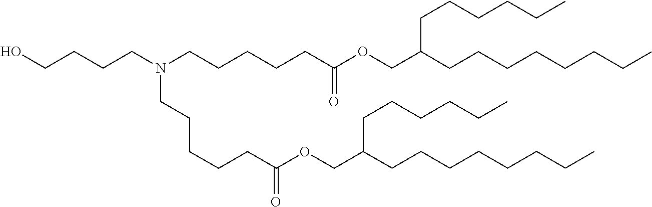 [SMILES: CCCCCCCCC(CCCCCC)COC(=O)CCCCCN(CCCCO)CCCCCC(=O)OCC(CCCCCC)CCCCCCCC] |
| III-20 | 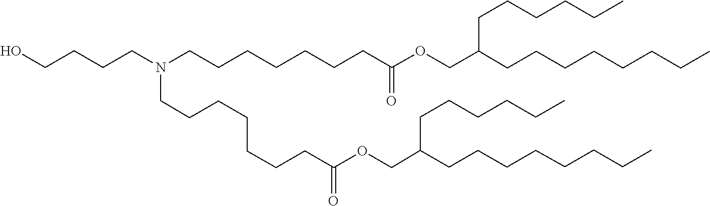 [SMILES: CCCCCCCCC(CCCCCC)COC(=O)CCCCCCCN(CCCCO)CCCCCCCC(=O)OCC(CCCCCC)CCCCCCCC] |
| III-21 | 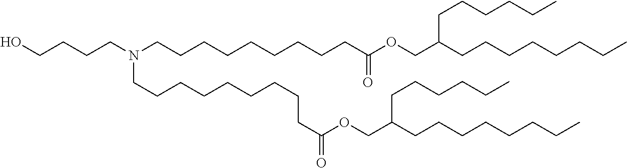 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 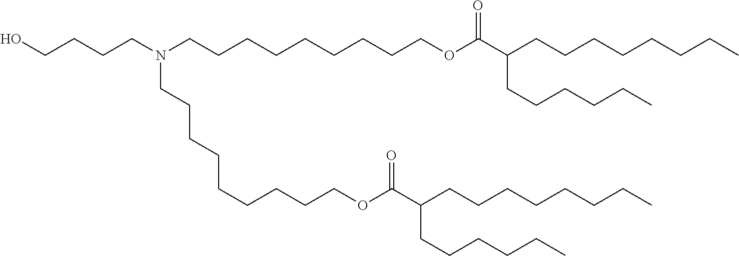 [SMILES: CCCCCCCCC(CCCCCC)C(=O)OCCCCCCCCCN(CCCCO)CCCCCCCCCOC(=O)C(CCCCCC)CCCCCCCC] |
| III-23 | 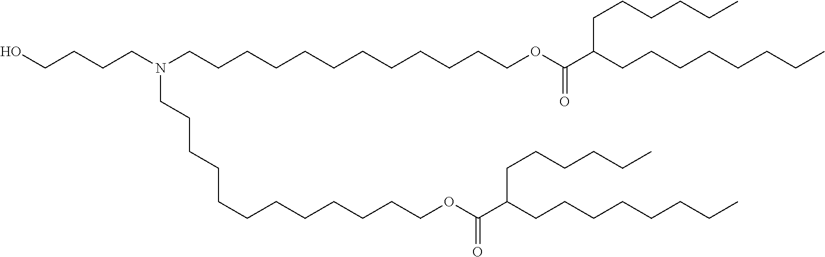 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 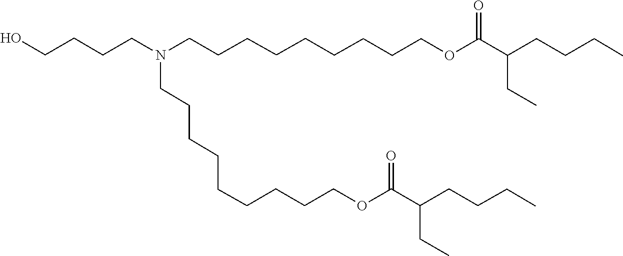 [SMILES: CCCCC(CC)C(=O)OCCCCCCCCCN(CCCCO)CCCCCCCCCOC(=O)C(CC)CCCC] |
| III-25 | 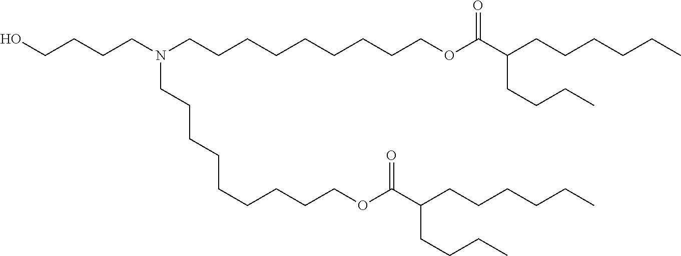 [SMILES: CCCCCCC(CCCC)C(=O)OCCCCCCCCCN(CCCCO)CCCCCCCCCOC(=O)C(CCCC)CCCCCC] |
| III-26 | 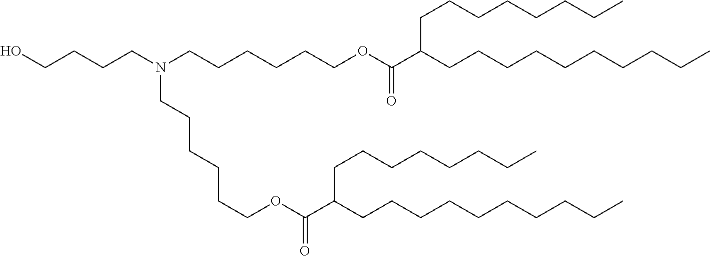 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 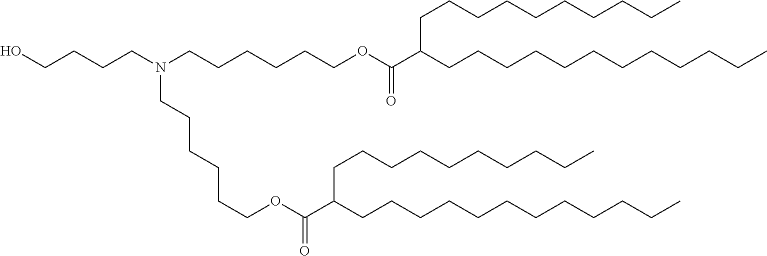 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 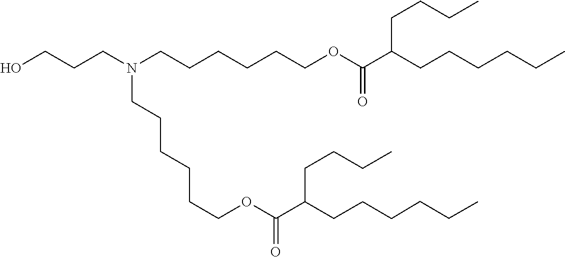 [SMILES: CCCCCCC(CCCC)C(=O)OCCCCCCN(CCCO)CCCCCCOC(=O)C(CCCC)CCCCCC] |
| III-29 | 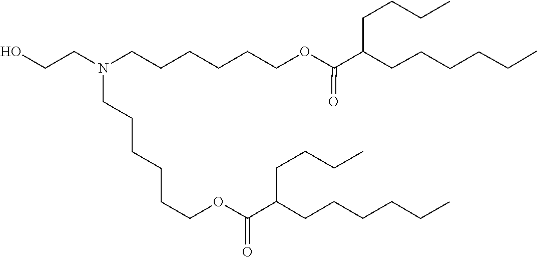 [SMILES: CCCCCCC(CCCC)C(=O)OCCCCCCN(CCO)CCCCCCOC(=O)C(CCCC)CCCCCC] |
| III-30 | 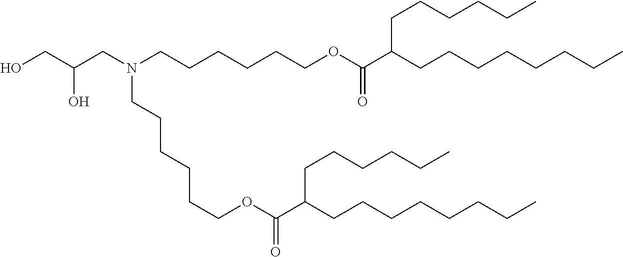 [SMILES: CCCCCCCCC(CCCCCC)C(=O)OCCCCCCN(CCCCCCOC(=O)C(CCCCCC)CCCCCCCC)CC(O)CO] |
| III-31 | 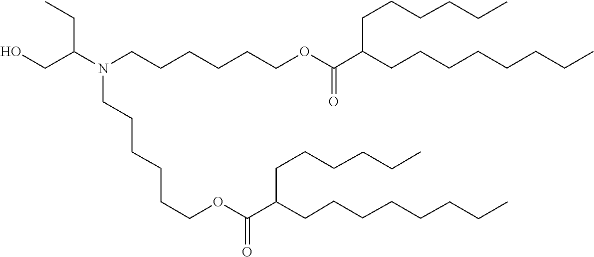 [SMILES: CCCCCCCCC(CCCCCC)C(=O)OCCCCCCN(CCCCCCOC(=O)C(CCCCCC)CCCCCCCC)C(CC)CO] |
| III-32 | 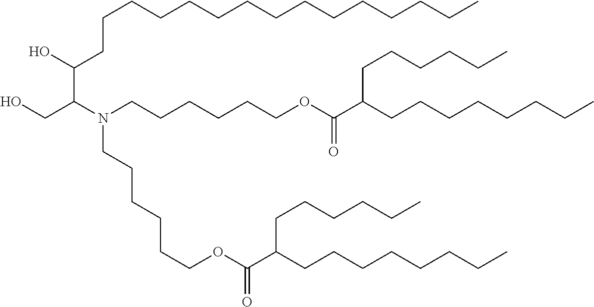 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 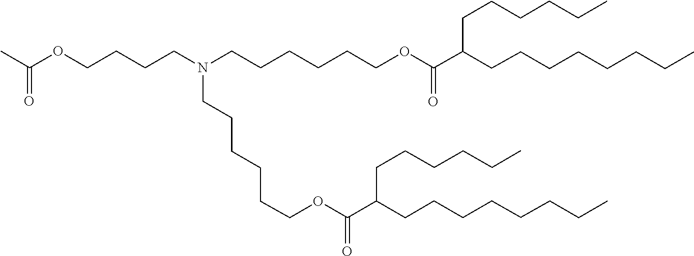 [SMILES: CCCCCCCCC(CCCCCC)C(=O)OCCCCCCN(CCCCCCOC(=O)C(CCCCCC)CCCCCCCC)CCCCOC(C)=O] |
| III-34 | 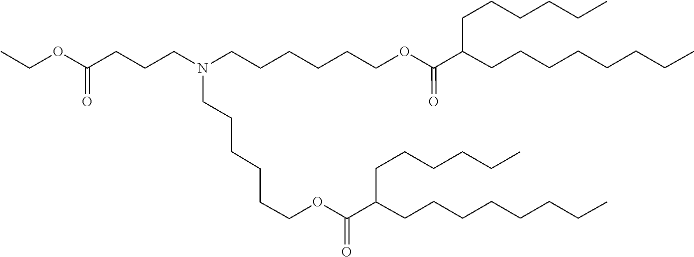 [SMILES: CCCCCCCCC(CCCCCC)C(=O)OCCCCCCN(CCCCCCOC(=O)C(CCCCCC)CCCCCCCC)CCCC(=O)OCC] |
| III-35 | 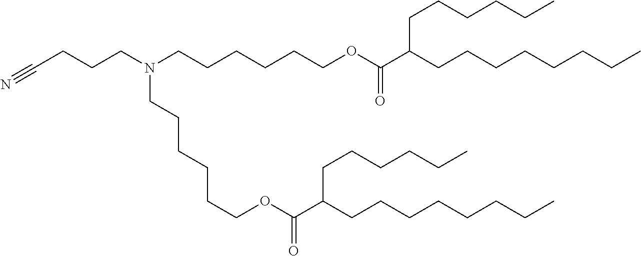 [SMILES: CCCCCCCCC(CCCCCC)C(=O)OCCCCCCN(CCCC#N)CCCCCCOC(=O)C(CCCCCC)CCCCCCCC] |
| III-36 | 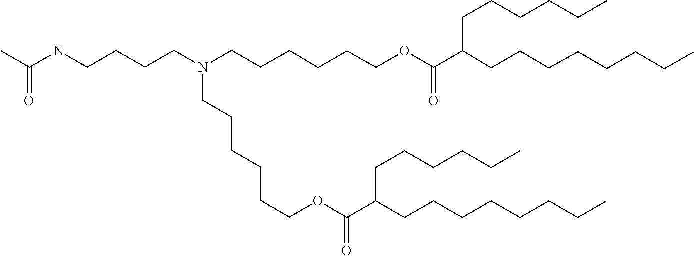 |

In some embodiments, the LNP comprises a lipid of Formula (III), RNA, a neutral lipid, a steroid and a pegylated lipid. In some embodiments, the lipid of Formula (III) is compound III-3. In some embodiments, the neutral lipid is DSPC. In some embodiments, the steroid is cholesterol. In some embodiments, the pegylated lipid is ALC-0159. In some embodiments, the cationic lipid is present in the LNP in an amount from about 40 to about 50 mole percent. In one embodiment, the neutral lipid is present in the LNP in an amount from about 5 to about 15 mole percent. In one embodiment, the steroid is present in the LNP in an amount from about 35 to about 45 mole percent. In one embodiment, the pegylated lipid is present in the LNP in an amount from about 1 to about 10 mole percent. In some embodiments, the LNP comprises compound III-3 in an amount from about 40 to about 50 mole percent, DSPC in an amount from about 5 to about 15 mole percent, cholesterol in an amount from about 35 to about 45 mole percent, and ALC-0159 in an amount from about 1 to about 10 mole percent.

In some embodiments, the LNP comprises compound III-3 in an amount of about 47.5 mole percent, DSPC in an amount of about 10 mole percent, cholesterol in an amount of about 40.7 mole percent, and ALC-0159 in an amount of about 1.8 mole percent.

In various different embodiments, the cationic lipid has one of the structures set forth in the table below.

TABLE 26
Representative cationic lipids.

| No. | Structure |
|---|---|
| A | 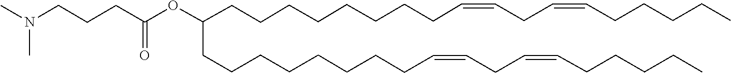 [SMILES: CCCCC/C=C\C/C=C\CCCCCCCCC(CCCCCCCC/C=C\C/C=C\CCCCC)OC(=O)CCCN(C)C] |
| B | 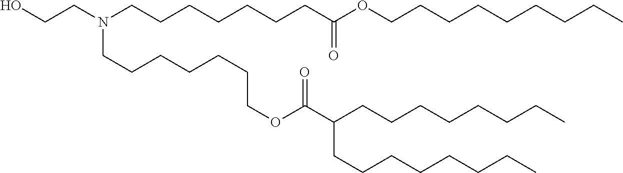 [SMILES: CCCCCCCCCOC(=O)CCCCCCCN(CCO)CCCCCCCOC(=O)C(CCCCCCCC)CCCCCCCC] |
| C | 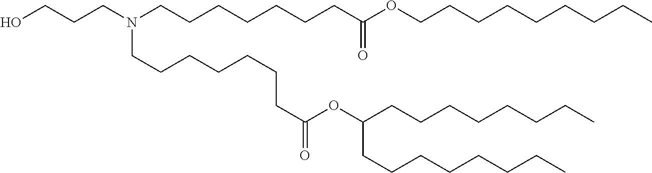 [SMILES: CCCCCCCCCOC(=O)CCCCCCCN(CCCO)CCCCCCCC(=O)OC(CCCCCCCC)CCCCCCCC] |
| D | 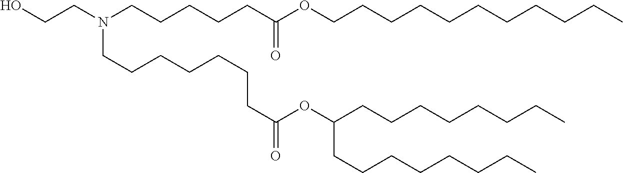 [SMILES: CCCCCCCCCCCOC(=O)CCCCCN(CCO)CCCCCCCC(=O)OC(CCCCCCCC)CCCCCCCC] |
| E | 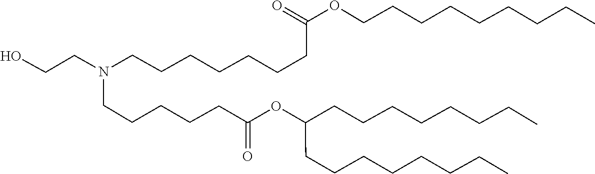 [SMILES: CCCCCCCCCOC(=O)CCCCCCCN(CCO)CCCCCC(=O)OC(CCCCCCCC)CCCCCCCC] |
| F | 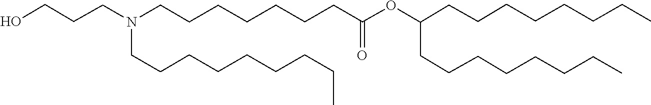 [SMILES: CCCCCCCCCN(CCCO)CCCCCCCC(=O)OC(CCCCCCCC)CCCCCCCC] |

In some embodiments, the LNP comprises a cationic lipid shown in the above table, e.g., a cationic lipid of Formula (B) or Formula (D), in particular a cationic lipid of Formula (D), RNA, a neutral lipid, a steroid and a pegylated lipid. In some embodiments, the neutral lipid is DSPC. In some embodiments, the steroid is cholesterol. In some embodiments, the pegylated lipid is DMG-PEG 2000.

In one embodiment, the LNP comprises a cationic lipid that is an ionizable lipid-like material (lipidoid). In one embodiment, the cationic lipid has the following structure:

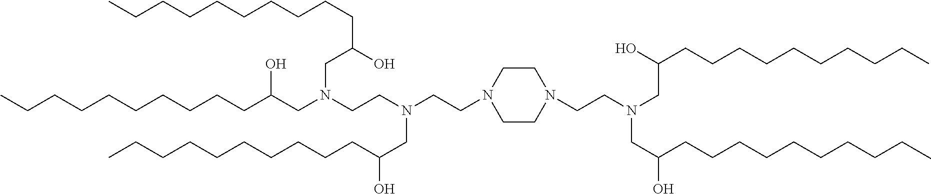 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

The N/P value is preferably at least about 4. In some embodiments, the N/P value ranges from 4 to 20, 4 to 12, 4 to 10, 4 to 8, or 5 to 7. In one embodiment, the N/P value is about 6.

LNP described herein may have an average diameter that in one embodiment ranges from about 30 nm to about 200 nm, or from about 60 nm to about 120 nm.

### RNA Targeting

Some aspects of the present disclosure involve the targeted delivery of RNA disclosed herein (e.g., RNA encoding vaccine antigens and/or immunostimulants).

In one embodiment, the present disclosure involves targeting lung. Targeting lung is in particular preferred if the RNA administered is RNA encoding vaccine antigen. RNA may be delivered to lung, for example, by administering the RNA which may be formulated as particles as described herein, e.g., lipid particles, by inhalation.

In one embodiment, the present disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of RNA lipoplex particles, RNA accumulation and/or RNA expression in the spleen occurs. Thus, RNA lipoplex particles of the present disclosure may be used for expressing RNA in the spleen. In an embodiment, after administration of RNA lipoplex particles, no or essentially no RNA accumulation and/or RNA expression in the lung and/or liver occurs. In one embodiment, after administration of RNA lipoplex particles, RNA accumulation and/or RNA expression in antigen presenting cells, such as professional antigen presenting cells in the spleen occurs. Thus, RNA lipoplex particles of the present disclosure may be used for expressing RNA in such antigen presenting cells. In one embodiment, the antigen presenting cells are dendritic cells and/or macrophages.

The electric charge of RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2, or about 1.6:2 to about 1:2, or about 1.6:2 to about 1.1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

Immunostimulants may be provided to a subject by administering to the subject RNA encoding an immunostimulant in a formulation for preferential delivery of RNA to liver or liver tissue. The delivery of RNA to such target organ or tissue is preferred, in particular, if it is desired to express large amounts of the immunostimulant and/or if systemic presence of the immunostimulant, in particular in significant amounts, is desired or required.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses.

Examples of suitable immunostimulants for targeting liver are cytokines involved in T cell proliferation and/or maintenance. Examples of suitable cytokines include IL2 or IL7, fragments and variants thereof, and fusion proteins of these cytokines, fragments and variants, such as extended-PK cytokines.

In another embodiment, RNA encoding an immunostimulant may be administered in a formulation for preferential delivery of RNA to the lymphatic system, in particular secondary lymphoid organs, more specifically spleen. The delivery of an immunostimulant to such target tissue is preferred, in particular, if presence of the immunostimulant in this organ or tissue is desired (e.g., for inducing an immune response, in particular in case immunostimulants such as cytokines are required during T-cell priming or for activation of resident immune cells), while it is not desired that the immunostimulant is present systemically, in particular in significant amounts (e.g., because the immunostimulant has systemic toxicity).

Examples of suitable immunostimulants are cytokines involved in T cell priming. Examples of suitable cytokines include IL12, IL15, IFN-α, or IFN-β, fragments and variants thereof, and fusion proteins of these cytokines, fragments and variants, such as extended-PK cytokines.

### Immunostimulants

In one embodiment, RNA encoding vaccine antigen may be non-immunogenic. In this and other embodiments, RNA encoding a vaccine antigen may be co-administered with an immunostimulant or RNA encoding an immunostimulant. The methods and agents described herein are particularly effective if the immunostimulant is attached to a pharmacokinetic modifying group (hereafter referred to as “extended-pharmacokinetic (PK)” immunostimulant). The methods and agents described herein are particularly effective if the immunostimulant is administered in the form of RNA encoding an immunostimulant. In one embodiment, said RNA is targeted to the liver for systemic availability. Liver cells can be efficiently transfected and are able to produce large amounts of protein.

An “immunostimulant” is any substance that stimulates the immune system by inducing activation or increasing activity of any of the immune system's components, in particular immune effector cells. The immunostimulant may be pro-inflammatory.

According to one aspect, the immunostimulant is a cytokine or a variant thereof. Examples of cytokines include interferons, such as interferon-alpha (IFN-α) or interferon-gamma (IFN-γ), interleukins, such as IL2, IL7, IL12, IL15 and IL23, colony stimulating factors, such as M-CSF and GM-CSF, and tumor necrosis factor. According to another aspect, the immunostimulant includes an adjuvant-type immunostimulatory agent such as APC Toll-like Receptor agonists or costimulatory/cell adhesion membrane proteins. Examples of Toll-like Receptor agonists include costimulatory/adhesion proteins such as CD80, CD86, and ICAM-1.

Cytokines are a category of small proteins (˜5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumour necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

According to the present disclosure, a cytokine may be a naturally occurring cytokine or a functional fragment or variant thereof. A cytokine may be human cytokine and may be derived from any vertebrate, especially any mammal. One particularly preferred cytokine is interferon-α.

### Interferons

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses.

Based on the type of receptor through which they signal, interferons are typically divided among three classes: type I interferon, type II interferon, and type III interferon.

All type I interferons bind to a specific cell surface receptor complex known as the IFN-α/β receptor (IFNAR) that consists of IFNAR1 and IFNAR2 chains.

The type I interferons present in humans are IFNα, IFNβ, IFNε, IFNκ and IFNω. In general, type I interferons are produced when the body recognizes a virus that has invaded it. They are produced by fibroblasts and monocytes.

Once released, type I interferons bind to specific receptors on target cells, which leads to expression of proteins that will prevent the virus from producing and replicating its RNA and DNA.

The IFNα proteins are produced mainly by plasmacytoid dendritic cells (pDCs). They are mainly involved in innate immunity against viral infection. The genes responsible for their synthesis come in 13 subtypes that are called IFNA1, IFNA2, IFNA4, IFNA5, IFNA6, IFNA7, IFNA8, IFNA10, IFNA13, IFNA14, IFNA16, IFNA17, IFNA21. These genes are found together in a cluster on chromosome 9.

The IFNβ proteins are produced in large quantities by fibroblasts. They have antiviral activity that is involved mainly in innate immune response. Two types of IFNβ have been described, IFNβ1 and IFNβ3. The natural and recombinant forms of IFNβ1 have antiviral, antibacterial, and anticancer properties.

Type II interferon (IFNγ in humans) is also known as immune interferon and is activated by IL12. Furthermore, type II interferons are released by cytotoxic T cells and T helper cells.

Type III interferons signal through a receptor complex consisting of IL10R2 (also called CRF2-4) and IFNLR1 (also called CRF2-12). Although discovered more recently than type I and type II IFNs, recent information demonstrates the importance of type III IFNs in some types of virus or fungal infections.

In general, type I and II interferons are responsible for regulating and activating the immune response.

According to the present disclosure, a type I interferon is preferably IFNα or IFNβ, more preferably IFNα.

According to the present disclosure, an interferon may be a naturally occurring interferon or a functional fragment or variant thereof. An interferon may be human interferon and may be derived from any vertebrate, especially any mammal.

### Interleukins

Interleukins (ILs) are a group of cytokines (secreted proteins and signal molecules) that can be divided into four major groups based on distinguishing structural features. However, their amino acid sequence similarity is rather weak (typically 15-25% identity). The human genome encodes more than 50 interleukins and related proteins. According to the present disclosure, an interleukin may be a naturally occurring interleukin or a functional fragment or variant thereof. An interleukin may be human interleukin and may be derived from any vertebrate, especially any mammal.

### Extended-PK Group

Immunostimulant polypeptides described herein can be prepared as fusion or chimeric polypeptides that include an immunostimulant portion and a heterologous polypeptide (i.e., a polypeptide that is not an immunostimulant). The immunostimulant may be fused to an extended-PK group, which increases circulation half-life. Non-limiting examples of extended-PK groups are described infra. It should be understood that other PK groups that increase the circulation half-life of immunostimulants such as cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16(7):903-15 which is herein incorporated by reference in its entirety. As used herein, an “extended-PK” immunostimulant refers to an immunostimulant moiety in combination with an extended-PK group. In one embodiment, the extended-PK immunostimulant is a fusion protein in which an immunostimulant moiety is linked or fused to an extended-PK group.

In certain embodiments, the serum half-life of an extended-PK immunostimulant is increased relative to the immunostimulant alone (i.e., the immunostimulant not fused to an extended-PK group). In certain embodiments, the serum half-life of the extended-PK immunostimulant is at least 20, 40, 60, 80, 100, 120, 150, 180, 200, 400, 600, 800, or 1000% longer relative to the serum half-life of the immunostimulant alone. In certain embodiments, the serum half-life of the extended-PK immunostimulant is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the immunostimulant alone. In certain embodiments, the serum half-life of the extended-PK immunostimulant is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

As used herein, “half-life” refers to the time taken for the serum or plasma concentration of a compound such as a peptide or protein to reduce by 50%, in vivo, for example due to degradation and/or clearance or sequestration by natural mechanisms. An extended-PK immunostimulant suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular an immunostimulant. The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In one embodiment, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver, that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the present disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used. In other embodiments, the albumin fusion protein has a therapeutic protein fused to both the N-terminus and the C-terminus of albumin. In a preferred embodiment, the therapeutic proteins fused at the N- and C-termini are the same therapeutic proteins. In another preferred embodiment, the therapeutic proteins fused at the N- and C-termini are different therapeutic proteins. In one embodiment, the different therapeutic proteins are both cytokines.

In one embodiment, the therapeutic protein(s) is (are) joined to the albumin through (a) peptide linker(s). A linker peptide between the fused portions may provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

In certain aspects, the extended-PK immunostimulant, suitable for use according to the present disclosure, can employ one or more peptide linkers. As used herein, the term “peptide linker” refers to a peptide or polypeptide sequence which connects two or more domains (e.g., the extended-PK moiety and an immunostimulant moiety) in a linear amino acid sequence of a polypeptide chain. For example, peptide linkers may be used to connect an immunostimulant moiety to a HSA domain.

Linkers suitable for fusing the extended-PK group to e.g. an immunostimulant are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

In addition to, or in place of, the heterologous polypeptides described above, an immunostimulant polypeptide described herein can contain sequences encoding a “marker” or “reporter”. Examples of marker or reporter genes include β-lactamase, chloramphenicol acetyltransferase (CAT), adenosine deaminase (ADA), aminoglycoside phosphotransferase, dihydrofolate reductase (DHFR), hygromycin-B-phosphotransferase (HPH), thymidine kinase (TK), β-galactosidase, and xanthine guanine phosphoribosyltransferase (XGPRT).

### Pharmaceutical Compositions

Agents described herein may be administered in pharmaceutical compositions or medicaments and may be administered in the form of a suitable pharmaceutical composition.

In some embodiments, the pharmaceutical composition described herein is an immunogenic composition for inducing immune responses against antigenic polypeptides associated with at least two infectious agents (including, e.g., at least three infectious agents). In some embodiments, at least two infectious agents may comprise a coronavirus and a non-coronvirus respiratory virus. In some embodiments, at least two infectious agents may comprise a coronavirus and an influenza virus. In some embodiments, at least two infectious agents may comprise a coronavirus, an influenza virus, and a respiratory syncytial virus.

In some embodiments, a pharmaceutical composition may comprise two or more distinct compositions—for example a first composition that is immunogenic with respect to a first infectious agent (e.g., SARS-CoV-2) and a second composition that is immunogenic with respect to a second infectious agent (e.g., influenza).

Alternatively or additionally, in some embodiments, a pharmaceutical composition (and, in some embodiments, an individual composition of a plurality of compositions that together comprise a pharmaceutical composition for use in accordance with the present disclosure) may comprise two or more active agents (e.g., two or more RNAs, each of which encodes a different immunogenic polypeptide).

In one embodiment, a pharmaceutical composition described herein is an immunogenic composition for inducing an immune response against coronavirus and influenza in a subject. In some embodiments, such an immunogenic composition comprises one or more RNAs, each encoding one of more antigenic polypeptides associated with a coronavirus. In some embodiments, such an immunogenic composition comprises or delivers one or more antigenic polypeptides associated with one or more influenza viruses. In some embodiments, an immunogenic composition comprises a commercially approved influenza vaccine (e.g., in some embodiments, an immunogenic composition comprises (i) one or more RNAs, each encoding one or more antigenic polypeptides associated with a coronavirus, and (ii) a commercially approved influenza vaccine). In some embodiments, an immunogenic composition comprises (i) an inactivated influenza virus (e.g., Fluzone®, Fluzone high-dose Quadrivalent®, Fluzone Quadrivalent®, Fluzone intradermal Quardivalent®, Fluzone quadrivalent southern Hemisphere®, Fluad®, Fluad Quadrivalent®, Afluria Quardivalent®, Fluarix Quadrivalent®, FluLaval Quadrivalent®, or Flucelvax Quadrivalent®), a recombinant influenza vaccine (e.g., Flublok Quadrivalent®), a live attenuated influenza vaccine (e.g., FluMist Quadrivalent®), a non-adjuvanted influenza vaccine, an adjuvanted influenza vaccine, or a subunit or split vaccine, and (ii) one or more RNAs, each encoding one or more antigenic polypeptides associated with a coronavirus. In some embodiments, an immunogenic composition comprises (i) an inactivated influenza virus (e.g., Fluzone®, Fluzone high-dose Quadrivalent®, Fluzone Quadrivalent®, Fluzone intradermal Quardivalent®, Fluzone quadrivalent southern Hemisphere®, Fluad®, Fluad Quadrivalent®, Afluria Quardivalent®, Fluarix Quadrivalent®, FluLaval Quadrivalent®, or Flucelvax Quadrivalent®) and (ii) one or more RNAs, each encoding one or more antigenic polypeptides associated with a coronavirus. In some embodiments, an immunogenic composition comprises (i) Fluzone®, Fluzone high-dose Quadrivalent®, Fluzone Quadrivalent®, Fluzone intradermal Quardivalent®, or Fluzone quadrivalent southern Hemisphere® and (ii) one or more RNAS, each encoding one or more antigenic polypeptides associated with a coronavirus. In some embodiments, an immunogenic composition comprises (i) a recombinant influenza vaccine (e.g., Flublok Quadrivalent®), and (ii) one or more RNAs, each encoding one or more antigenic polypeptides associated with a coronavirus.

In one embodiment, a pharmaceutical composition described herein is an immunogenic composition for inducing an immune response against coronavirus and RSV in a subject. In some embodiments, an immunogenic composition comprises (i) an RSV vaccine known in the art (e.g., an RSV vaccine described herein) and (ii) one or more RNAS, each encoding one or more antigenic polypeptides associated with a coronavirus. In some embodiments, an immunogenic composition comprises (i) one or more RNAs, each encoding one or more antigenic polypeptides associated with a coronavirus, and (ii) an RSV vaccine (e.g., RSVpreF).

In one embodiment, a pharmaceutical composition described herein is an immunogenic composition for inducing an immune response against coronavirus, influenza, and RSV in a subject. In some embodiments, an immunogenic composition comprises (i) an RSV vaccine known in the art (e.g., an RSV vaccine described herein), (ii) one or more RNAs, each encoding one or more antigenic polypeptides associated with a coronavirus, and (iii) one or more RNAs, each encoding one or more antigenic polypeptides associated with an influenza virus. In some embodiments, an immunogenic composition comprises (i) an RSV vaccine known in the art (e.g., RSVpreF), (ii) one or more RNAS, each encoding one or more antigenic polypeptides associated with a coronavirus, and (iii) one or more RNAs, each encoding one or more antigenic polypeptides associated with an influenza virus. In some embodiments, an immunogenic composition comprises (i) an RSV vaccine known in the art (e.g., an RSV vaccine described herein), (ii) one or more RNAs, each encoding one or more antigenic polypeptides associated with a coronavirus, and (iii) a commercially available influenza vaccine (e.g., an influenza vaccine described herein).

In some embodiments, an immunogenic composition comprises (i) RSVpreF, (ii) one or more RNAs, each encoding one or more antigenic polypeptides associated with a coronavirus, and (iii) Fluzone®, Fluzone high-dose Quadrivalent®, Fluzone Quadrivalent®, Fluzone intradermal Quardivalent®, or Fluzone quadrivalent southern Hemisphere®. In some embodiments, an immunogenic composition comprises (i) RSVpreF, (ii) one or more RNAS, each encoding one or more antigenic polypeptides associated with a coronavirus, and (iii) a recombinant influenza vaccine (e.g., Flublok).

In some embodiments, an immunogenic composition comprises (i) one or more RNAs, each encoding one or more antigenic polypeptides associated with a coronavirus, and (ii) an RSV vaccine (e.g., RSVpreF).

In some embodiments, an immunogenic composition comprises one or more RNAs, each encoding one or more antigenic polypeptides associated with an influenza virus. In some embodiments, an immunogenic composition comprises one or more RNAs, each encoding one or more antigenic polypeptides associated with an influenza virus, where the one or more RNAs are described herein. In some embodiments, an immunogenic composition comprises one or more RNAs, each encoding one or more antigenic polypeptides associated with an influenza vaccine, that are known in the art (e.g., an RNA described in Feldman, Robert A., et al. “mRNA vaccines against H10N8 and H7N9 influenza viruses of pandemic potential are immunogenic and well tolerated in healthy adults in phase 1 randomized clinical trials.” Vaccine 37.25 (2019): 3326-3334, the contents of which are incorporated by reference herein in their entirety).

In embodiments in which an RSV vaccine is co-administered or co-formulated with (i) a SARS-CoV-2 vaccine or (ii) SARS-CoV-2 and influenza vaccines (e.g., embodiments in which an RSV vaccine (e.g., RSVpreF) is co-administered or co-formulated with (i) one or more RNAs, each encoding a SARS-CoV-2 S protein comprising one or more mutations associated with a SARS-CoV-2 strain or variant, or an immunogenic fragment thereof, (ii) one or more RNAs, each encoding a SARS-CoV-2 S protein comprising one or more mutations associated with a SARS-CoV-2 strain or variant, or an immunogenic fragment thereof, and one or more RNAs, each encoding an antigen of a different influenza strain, or (iii) one or more RNAs, each encoding a SARS-CoV-2 S protein comprising one or more mutations associated with a SARS-CoV-2 strain or variant, or an immunogenic fragment thereof, and a commercially available influenza vaccine (e.g., a commercially available vaccine described here)), the RSV vaccine can be administered at a dose that (a) is a standard or approved dose, (b) has been tested in a clinical trial, (c) is planned to be tested in a clinical trial, and/or (d) has shown efficacy in a clinical trial. For example, in some embodiments, RSVpreF can be co-administered or co-formulated in an amount of about 60 μg, about 120 μg, or about 240 μg of total antigen. In some embodiments, an RSV vaccine (e.g., RSVpreF) is administered with a SARS-CoV-2 vaccine (e.g., a SARS-CoV-2 bivalent vaccine described herein) in a combined amount of about 0.8 mL. In some embodiments, an RSV vaccine is co-administered to a subject 60 years and older. In some embodiments, an RSV vaccine is co-administered to a subject 65 years and older. In some embodiments, co-administration of an RSV vaccine (e.g., RSVpreF administered at a dose of about 60 μg, about 120 μg, or about 240 μg) with a SARS-CoV-2 vaccine described herein, or a SARS-CoV-2/influenza combination described herein results in an increase in neutralization titers against an RSV A and/or RSV B strain (e.g., as measured 28 days after administration). In some embodiments, co-administration of an RSV vaccine (e.g., RSVpreF administered at a dose of about 60 μg, about 120 μg, or about 240 μg) with a SARS-CoV-2 vaccine described herein, or a SARS-CoV-2/influenza combination described herein results in neutralization titers that are not inferior to those induced by the RSV vaccine, SARS-CoV-2 vaccine, or influenza vaccine administered separately. In some embodiments, an RSV vaccine (e.g., RSVpreF administered at a dose of about 60 μg, about 120 μg, or about 240 μg) is co-administered with a SARS-CoV-2 vaccine described herein, or a SARS-CoV-2/influenza combination described herein to a subject who has previously been administered 3 or more doses of a SARS-CoV-2 vaccine (e.g., an FDA-approved SARS-CoV-2 vaccine, e.g., as described herein), with the most recent dose being an updated booster vaccine that was administered at least about 2 months prior (e.g., at least 3 months, at least 5 months, at least 6 months, or at least 150 days prior).

In some embodiments, an immunogenic composition described herein comprises at least one RNA encoding an antigenic polypeptide associated with an influenza virus and comprises or delivers at least one antigenic polypeptide associated with a coronavirus. In some embodiments, an immunogenic composition comprises an approved or authorized SARS-CoV-2 vaccine (e.g., an mRNA-1273 vaccine, an Ad26.CoV2.S vaccine, a ChAdxOx1 vaccine, an NVX-CoV2373 vaccine, a CvnCoV vaccine, a GAM-COVID0Vac vaccine, a CoronaVac vaccine, a BBIBP-CorV vaccine, an Ad5-nCOV vaccine, a zf2001 vaccine, a SCB-2019 vaccine, a JNJ 78436735 vaccine, or other approved mRNA or adenovector vaccines, etc.).

In some embodiments, immunogenic compositions described herein are vaccines.

In one embodiment of all aspects of the present disclosure, the components described herein such as RNA encoding a vaccine antigen may be administered in a pharmaceutical composition which may comprise a pharmaceutically acceptable carrier and may optionally comprise one or more adjuvants, stabilizers etc. In one embodiment, the pharmaceutical composition is for therapeutic or prophylactic treatments, e.g., for use in treating or preventing a coronavirus infection.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, IL10, IL12, IFNα, IFNγ, GM-CSF, LT-a. Further known adjuvants are aluminium hydroxide, squalene, MF59, AddaVax, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration.

As used herein, “parenteral administration” refers to the administration in any manner other than through the gastrointestinal tract, such as by intravenous injection. In a preferred embodiment, the pharmaceutical composition is formulated for intramuscular administration. In another embodiment, the pharmaceutical composition is formulated for systemic administration, e.g., for intravenous administration.

The term “co-administering” as used herein means a process whereby different compounds or compositions (e.g., RNA encoding an antigen and RNA encoding an immunostimulant) are administered to the same patient. The different compounds or compositions may be administered simultaneously, at essentially the same time, or sequentially.

The pharmaceutical compositions and products described herein may be provided as a frozen concentrate for solution for injection, e.g., at a concentration of 0.50 mg/mL. In one embodiment, for preparation of solution for injection, a drug product is thawed and diluted with isotonic sodium chloride solution (e.g., 0.9% NaCl, saline), e.g., by a one-step dilution process. In some embodiments, bacteriostatic sodium chloride solution (e.g., 0.9% NaCl, saline) cannot be used as a diluent. In some embodiments, a diluted drug product is an off-white suspension. The concentration of the final solution for injection varies depending on the respective dose level to be administered. In one embodiment, administration is performed within 6 h after begin of preparation due to the risk of microbial contamination and considering the multiple-dose approach of the preparation process. In one embodiment, in this period of 6 h, two conditions are allowed: room temperature for preparation, handling and transfer as well as 2 to 8° C. for storage.

Compositions described herein may be shipped and/or stored under temperature-controlled conditions, e.g., temperature conditions of about 4-5° C. or below, about −20° C. or below, −70° C.±10° C. (e.g., −80° C. to −60° C.), e.g., utilizing a cooling system (e.g., that may be or include dry ice) to maintain the desired temperature. In one embodiment, compositions described herein are shipped in temperature-controlled thermal shippers. Such shippers may contain a GPS-enabled thermal sensor to track the location and temperature of each shipment. The compositions can be stored by refilling with, e.g., dry ice.

The present disclosure also provides, in some embodiments, a vessel comprising two or more recently admixed vaccines.

In some embodiments, a vessel is a container that is suitable for holding and/or administering a vaccine formulation (e.g., a container that does not cause substantial degradation of a vaccine product under suitable storage conditions, that can maintain sterility of a vaccine formulation, and/or that can be used to administer a vaccine formulation by injection). In some embodiments, a vessel is made primarily of glass or plastic. In some embodiments, a vessel is a vial (e.g., a glass or plastic vial). In some embodiments, a vessel is a syringe (e.g., a glass or plastic syringe). In some embodiments, a vessel is a dual-component vial system (e.g., an “ACT-O-VIAL” system).

As used herein, the term “admixture” refers to a combination produced by combining two or more drug products, and the term “admixed vaccines” refers to a combination produced by combining two or more vaccine formulations (e.g., a SARS-CoV-2 vaccine and an influenza vaccine; a SARS-CoV-2 vaccine and an RSV vaccine; an influenza vaccine and an RSV vaccine; or an influenza vaccine, a SARS-CoV-2 vaccine, and an RSV vaccine). In some embodiments, an admixture comprises two or more drug products that are not substantially mixed prior to administering to a subject. In some embodiments, an admixture comprises two or more drug products that are mixed prior to administering to a subject. In some embodiments, an admixture comprises two, three, four, or five drug products. In some embodiments, an admixture can be produced by combining two or more liquid drug products. In some embodiments, an admixture can be produced by (i) obtaining an amount of a first vaccine in a syringe, and (ii) using the same syringe, and without dispersing the first vaccine, obtaining an amount of a second vaccine (e.g., using a method similar to that shown in FIG. 3(A)). In some embodiments, an admixture can be produced by (i) obtaining an amount of a first vaccine in a syringe, and (ii) using the same syringe, and without dispersing the first vaccine, obtaining an amount of a second vaccine, and (iii) using the same syringe, and without dispersing the first or second vaccine, obtaining a third vaccine (e.g., using a method similar to that shown in FIG. 3(B)). In some embodiments, an admixture can be formed by resuspending a lyophilized drug product with a liquid drug product (e.g., reconstituting a lyophilized RSV vaccine with (i) a liquid SARS-CoV-2 vaccine, (ii) a liquid influenza vaccine, or (iii) a liquid SARS-CoV-2/influenza vaccine). In some embodiments, a vaccine admixture can be formed by (i) resuspending a lyophilized vaccine with an appropriate solvent (e.g., sterile water), and (ii) combining the resuspended vaccine with one or more liquid vaccines (e.g., by combining in a vial or combining in a syringe, using a method described herein). In some embodiments, an admixed vaccine comprises two or more vaccines, each in an amount that has previously been shown to induce an immune response, previously been shown to reduce infection and/or harmful symptoms associated with a disease, and/or that has been approved by a relevant regulatory body (e.g., the USFDA or the EMA) for vaccinating against a relevant disease.

In some embodiments, a lyophilized vaccine can be reconstituted and/or admixed using an appropriate dual component vial system. As used herein, a “dual component vial system” refers to a vial that comprises two or more compartments, each of which can be used to hold a drug product or an appropriate solvent, and which comprises a mechanism for mixing the two compartments. Such vial systems are known in the art, and include, e.g., the “ACT-O-VIAL” system, produced by Pfizer Inc. A description of the ACT-O-vial system, and appropriate use thereof is provided, e.g., at https_//www.youtube.com/watch?v=2bbcgIor0Jg.

In some embodiments, a lyophilized vaccine is resuspended in an appropriate solvent (e.g., sterile water or sterile buffered solution) using a dual component vial system and then admixed with one or more liquid or lyophilized vaccines (e.g., using methods described herein). In some embodiments, a lyophilized vaccine (e.g., an RSV vaccine) is admixed with one or more liquid vaccines (e.g., a SARS-CoV-2 vaccine and/or an influenza vaccine) using a dual component vial system (e.g., one or more lyophilized vaccines are held in a first compartment and one or more liquid vaccines are held in a second compartment, and the two are admixed by combining the contents of the two compartments using an appropriate mechanism of the dual component vial system).

In some embodiments, two or more vaccines are admixed using a dual-chamber vial (e.g., (i) a vial containing a first chamber containing one or more liquid vaccines, and (ii) a second chamber containing one or more liquid vaccines; or (ii) a vial containing a first chamber containing one or more liquid vaccines, and (ii) a second chamber containing one or more lyophilized vaccines).

In some embodiments, two or more vaccines are admixed by a clinician (e.g., by a clinician before administering to a subject). In some embodiments, recently admixed vaccines are combined by a clinician before administering to a subject (e.g., about 24 hours or less, about 12 hours or less, about 6 hours or less, about 4 hours or less, about 2 hours or less, about 30 minutes or less, about 20 minutes or less, about 15 minutes or less, about 10 minutes or less, about 5 minutes or less, or about 1 minute or less before administering to a subject). In some embodiments, recently admixed vaccines are combined the same day that a subject is to be administered the admixed vaccines. In some embodiments, for an admixed vaccine that comprises one or more vaccines that must be prepared (e.g., thawed, diluted, and/or resuspended) prior to administering to a subject, the two or more vaccines are combined within an amount of time that is not greater than the maximum amount of time the one or more vaccines have shown to be stable once prepared and/or the maximum amount of time the one or more vaccines have been approved to be stored following preparation. In some embodiments, an admixture comprises two or more vaccines that must be prepared prior to administering to a subject, and the admixture is combined within an amount of time that is not greater than the maximum amount of time that any one of the two or more vaccines have been shown to be stable once prepared and/or the maximum amount of time that any one of the two or more vaccines have been approved to be stored following preparation (e.g., if an admixture comprises a first vaccine and a second vaccine, each of which must be prepared prior to administering to a subject, where the first vaccine has been shown to be stable for at most 24 hours, and the second vaccine has been shown to be stable for at most 48 hours, a recently admixed combination comprising the first vaccine and the second vaccine is admixed 24 hours or less prior to administering to a subject).

The present disclosure also provides, in some embodiments, a method for simultaneously administering two or more vaccines (e.g., SARS-CoV-2 and influenza, SARS-CoV-2 and RSV; or SARS-CoV-2, influenza, and RSV). In some embodiments, such methods result in vaccinating against and/or inducing an immune response against two or more diseases. In some embodiments, as used herein, two or more vaccines are considered to have been administered simultaneously if they are administered in a single shot. In some embodiments, two or more vaccines can be administered simultaneously using a method similar to that shown in FIG. 3. In some embodiments, two or more vaccines can be administered simultaneously using a vessel that can hold two or more vaccines in separate compartments, and which also has means for administering both vaccines to a subject (e.g., a dual barrel syringe (e.g., as described herein)).

### Treatments

The present disclosure provides methods and agents for inducing an adaptive immune response against coronavirus, influenza virus, or RSV, or any combination thereof (including, e.g., coronavirus and influenza virus, coronavirus and RSV, and coronavirus, influenza virus, and RSV) in a subject comprising administering an effective amount of a composition comprising RNA encoding a coronavirus vaccine antigen and RNA encoding an influenza antigen described herein.

In some embodiments, methods, compositions, or combinations described herein are administered to an older adult (e.g., a subject 60 years and older, or 65 years and older) at increased risk of severe disease caused by RSV infection (e.g., having one of the risk factors described herein). In some embodiments, methods, compositions, or combinations described herein are administered to an older adult (e.g., a subject 60 years and older, or 65 years and older) at increased risk of severe disease caused by RSV infection (e.g., having one of the risk factors described herein). In some embodiments, methods, compositions, or combinations described herein are administered to an infant or young child at increased risk of severe disease caused by RSV infection (e.g., having one of the risk factors described herein). In some embodiments, methods, compositions, or combinations described herein are administered to a subject having a condition or that can be exacerbated by RSV infection (e.g., having one of the conditions described herein).

In one embodiment, the methods and agents described herein provide immunity in a subject to coronavirus, coronavirus infection, or to a disease or disorder associated with coronavirus and/or to influenza, influenza infection, or to a disease or disorder associated with influenza. The present disclosure thus provides methods and agents for treating or preventing the infection, disease, or disorder associated with coronavirus and/or influenza.

In one embodiment, the methods and agents described herein are administered to a subject having an infection, disease, or disorder associated with coronavirus and/or influenza. In one embodiment, the methods and agents described herein are administered to a subject at risk for developing the infection, disease, or disorder associated with a coronavirus and/or an influenza virus. For example, the methods and agents described herein may be administered to a subject who is at risk for being in contact with a coronavirus and/or an influenza virus. In one embodiment, the methods and agents described herein are administered to a subject who lives in, traveled to, or is expected to travel to a geographic region in which coronavirus and/or influenza is prevalent. In one embodiment, the methods and agents described herein are administered to a subject who is in contact with or expected to be in contact with another person who lives in, traveled to, or is expected to travel to a geographic region in which coronavirus and/or influenza is prevalent. In one embodiment, the methods and agents described herein are administered to a subject who has knowingly been exposed to coronavirus and/or influenza through their occupation, or other contact. In one embodiment, a coronavirus is SARS-CoV-2. In some embodiments, methods and agents described herein are administered to a subject with evidence of prior exposure to and/or infection with SARS-CoV-2 and/or influenza and/or an antigen or epitope thereof or cross-reactive therewith. For example, in some embodiments, methods and agents described herein are administered to a subject in whom antibodies, B cells, and/or T cells reactive with one or more epitopes of a SARS-CoV-2 spike protein and/or an HA protein are detectable and/or have been detected.

For a composition to be useful as a vaccine, the composition must induce an immune response against a disease antigen (e.g., a coronavirus antigen and/or an influenza antigen) in a cell, tissue or subject (e.g., a human). In some embodiments, a composition induces an immune response against a coronavirus antigen in a cell, tissue or subject (e.g., a human). In some embodiments, a composition induces an immune response against an influenza antigen in a cell, tissue or subject (e.g., a human). In some instances, the vaccine induces a protective immune response in a mammal. The therapeutic compounds or compositions of the present disclosure may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present disclosure, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The term “dose” as used herein refers in general to a “dose amount” which relates to the amount of RNA administered per administration, i.e., per dosing.

In some embodiments, administration of an immunogenic composition or vaccine of the present disclosure may be performed by single administration or boosted by multiple administrations.

In some embodiments, a regimen described herein includes at least one dose. In some embodiments, a regimen includes a first dose and at least one subsequent dose. In some embodiments, the first dose is the same amount as at least one subsequent dose. In some embodiments, the first dose is the same amount as all subsequent doses. In some embodiments, the first dose is a different amount as at least one subsequent dose. In some embodiments, the first dose is a different amount than all subsequent doses. In some embodiments, a regimen comprises two doses. In some embodiments, a provided regimen consists of two doses. In some embodiments, a regimen comprises three doses.

In some embodiments, a provided regimen (e.g., a primary regimen or a booster regimen) comprises two doses, wherein the two doses are administered about 3 weeks apart from one another. In some embodiments, a provided regimen (e.g., a primary regimen or a booster regimen) comprises two doses, wherein the two doses are administered about 8 weeks apart from one another.

In some embodiments, a provided regimen (e.g., a primary regimen or a booster regimen) comprises three doses, where the second dose is adminstered about 3 weeks after the first dose, and the third dose is administered about 8 weeks after the second dose. In some embodiments a provided regimen (e.g., a primary regimen or a booster regimen) comprises four doses, where the second dose is adminstered about 3 weeks after the first dose, the third dose is administered about 8 weeks after the second dose, and the fourth dose is administered about 3 months or more (e.g., 6 months or more) after the third dose.

In one embodiment, the present disclosure envisions administration of a single dose. In one embodiment, the present disclosure envisions administration of a priming dose followed by one or more booster doses. The booster dose or the first booster dose may be administered 7 to 28 days or 14 to 24 days following administration of the priming dose. In some embodiments, a first booster dose may be administered 1 week to 3 months (e.g., 1 week, 2 weeks, 3 weeks, 4 weeks, 5 weeks, 6 weeks, 7 weeks, 8 weeks, 9 weeks, 10 weeks, 11 weeks, 12 weeks) following administration of a priming dose. In some embodiments, a subsequent booster dose may be administered at least 1 week or longer, including, e.g., at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, or longer, following a preceding booster dose. In some embodiments, subsequent booster doses may be administered about 5-9 weeks or 6-8 weeks apart. In some embodiments, at least one subsequent booster dose (e.g., after a first booster dose) may be administered at least 3 months or longer, including, e.g., at least 4 months, at least 5 months, at least 6 months, at least 7 months, at least 8 months, at least 9 months, at least 10 months, or longer, following a preceding dose.

In some embodiments, a dose comprises a total amount of RNA of 0.1 μg to 300 μg, 0.5 μg to 200 μg, or 1 μg to 100 μg, such as about 1 μg, about 2 μg, about 3 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg. In some embodiments, a dose comprises a total amount of RNA (e.g., modRNA) of up to about 100 μg. In some embodiments, a dose comprises 0.1 μg to 100 μg of one or more first RNAs and 0.1 μg to 100 μg of one or more second RNAs, wherein the one or more first RNAs each comprise a nucleotide sequence encoding an antigenic polypeptide associated with a first infectious agent (e.g., a coronavirus), and the one or more second RNAs each comprise a nucleotide sequence encoding an antigenic polypeptide associated with a second infectious agent (e.g., influenza). In some embodiments, a dose comprises 3 to 60 μg of one or more first RNAs and 3 to 90 μg of one or more second RNAs. In some embodiments, a dose comprises 3 to 60 μg of one or more first RNAs and 3 to 90 μg of one or more second RNAs, wherein the dose comprises up to 100 μg of RNA total. In some embodiments, a dose comprises 3 to 30 μg of one or more first RNAs and 3 to 60 μg of one or more second RNAs, wherein the dose comprises up to 100 μg of RNA total. In some embodiments, a dose comprises 3 μg of one or more first RNAs and 3 μg of one or more second RNAs. In some embodiments, a dose comprises 3 μg of one or more first RNAs and 6 μg of one or more second RNAs. In some embodiments, a dose comprises 10 μg of one or more first RNAs and 10 μg of one or more second RNAs. In some embodiments, a dose comprises 10 μg of one or more first RNAs and 20 μg of one or more second RNAs. In some embodiments, a dose comprises 30 μg of one or more first RNAs and 30 μg of one or more second RNAs. In some embodiments, a dose comprises 30 μg of one or more first RNAs and 60 μg of one or more second RNAs. In some embodiments, a dose comprises 60 μg of one or more first RNAs and 30 μg of one or more second RNAs.

In some embodiments, a dose comprises a monovalent or bivalent SARS-CoV-2 RNA vaccine comprising 3 to 60 μg of total RNA and a tetravalent influenza RNA vaccine comprising 3 to 60 μg of total RNA. In some embodiments, a dose comprises a monovalent or bivalent SARS-CoV-2 RNA vaccine and a tetravalent influenza RNA vaccine, and comprises 100 μg of total RNA. In some embodiments, a dose comprises a monovalent or bivalent SARS-CoV-2 RNA vaccine and a tetravalent influenza RNA vaccine, and comprises about 90 μg of total RNA. In some embodiments, a dose comprises a monovalent or bivalent SARS-CoV-2 RNA vaccine and a tetravalent influenza RNA vaccine, and comprises about 60 μg of total RNA. In some embodiments, a dose comprises about a monovalent or bivalent SARS-CoV-2 RNA vaccine comprising 30 μg of total RNA and a tetravalent influenza RNA vaccine comprising 30 μg of total RNA. In some embodiments, a dose comprises a monovalent or bivalent SARS-CoV-2 RNA vaccine comprising 30 μg of total RNA and a tetravalent influenza RNA vaccine comprising 60 μg of total RNA. In some embodiments, a dose comprises a monovalent or bivalent SARS-CoV-2 RNA vaccine comprising 60 μg of total RNA and a tetravalent influenza RNA vaccine comprising about 30 μg of total RNA.

In some embodiments, a dose comprises a monovalent or bivalent SARS-CoV-2 RNA vaccine comprising 3 μg of total RNA (e.g., 3 μg of an RNA encoding a SARS-CoV-2 S protein of an Omicron variant (e.g., a BA.1, BA.4/5, or XBB.1.5 variant), or 1.5 μg of an RNA encoding a SARS-CoV-2 S protein of a Wuhan strain and 1.5 μg of an RNA encoding a SARS-CoV-2 S protein of an Omicron variant (e.g., a BA.1, BA.4/5, or XBB.1.5 Omicron variant)) and a tetravalent influenza RNA vaccine comprising 3 μg of total RNA (e.g., 0.75 μg of an RNA encoding an HA protein of an H1N1 influenza strain, 0.75 μg of an RNA encoding an HA protein of an H3N2 influenza strain, 0.75 μg of an RNA encoding an HA protein of a B/Victoria influenza lineage, and 0.75 μg of an RNA encoding an HA protein of a B/Yamagata influenza lineage).

In some embodiments, a dose comprises a monovalent or bivalent SARS-CoV-2 RNA vaccine comprising 3 μg of total RNA (e.g., 3 μg of an RNA encoding a SARS-CoV-2 S protein of an Omicron variant (e.g., a BA.1, BA.4/5, or XBB.1.5 variant), or 1.5 μg of an RNA encoding a SARS-CoV-2 S protein of a Wuhan strain and 1.5 μg of an RNA encoding a SARS-CoV-2 S protein of an Omicron variant (e.g., a BA.1, BA.4/5, or XBB.1.5 Omicron variant)) and a tetravalent influenza RNA vaccine comprising 6 μg of total RNA (e.g., 1.5 μg of an RNA encoding an HA protein of an H1N1 influenza strain, 1.5 μg of an RNA encoding an HA protein of an H3N2 influenza strain, 1.5 μg of an RNA encoding an HA protein of a B/Victoria influenza lineage, and 1.5 μg of an RNA encoding an HA protein of a B/Yamagata influenza lineage).

In some embodiments, a dose comprises a monovalent or bivalent SARS-CoV-2 RNA vaccine comprising 6 μg of total RNA (e.g., 6 μg of an RNA encoding a SARS-CoV-2 S protein of an Omicron variant (e.g., a BA.1, BA.4/5, or XBB.1.5 variant), or 3 μg of an RNA encoding a SARS-CoV-2 S protein of a Wuhan strain and 3 μg of an RNA encoding a SARS-CoV-2 S protein of an Omicron variant (e.g., a BA.1, BA.4/5, or XBB.1.5 Omicron variant)) and a tetravalent influenza RNA vaccine comprising 3 μg of total RNA (e.g., 0.75 μg of an RNA encoding an HA protein of an H1N1 influenza strain, 0.75 μg of an RNA encoding an HA protein of an H3N2 influenza strain, 0.75 μg of an RNA encoding an HA protein of a B/Victoria influenza lineage, and 0.75 μg of an RNA encoding an HA protein of a B/Yamagata influenza lineage).

In some embodiments, a dose comprises a monovalent or bivalent SARS-CoV-2 RNA vaccine comprising 10 μg of total RNA (e.g., 10 μg of an RNA encoding a SARS-CoV-2 S protein of an Omicron variant (e.g., a BA.1, BA.4/5, or XBB.1.5 variant), or 5 μg of an RNA encoding a SARS-CoV-2 S protein of a Wuhan strain and 5 μg of an RNA encoding a SARS-CoV-2 S protein of an Omicron variant (e.g., a BA.1, BA.4/5, or XBB.1.5 Omicron variant)) and a tetravalent influenza RNA vaccine comprising 10 μg of total RNA (e.g., 2.5 μg of an RNA encoding an HA protein of an H1N1 influenza strain, 2.5 μg of an RNA encoding an HA protein of an H3N2 influenza strain, 2.5 μg of an RNA encoding an HA protein of a B/Victoria influenza lineage, and 2.5 μg of an RNA encoding an HA protein of a B/Yamagata influenza lineage).

In some embodiments, a dose comprises a monovalent or bivalent SARS-CoV-2 RNA vaccine comprising 10 μg of total RNA (e.g., 10 μg of an RNA encoding a SARS-CoV-2 S protein of an Omicron variant (e.g., a BA.1, BA.4/5, or XBB.1.5 variant), or 5 μg of an RNA encoding a SARS-CoV-2 S protein of a Wuhan strain and 5 μg of an RNA encoding a SARS-CoV-2 S protein of an Omicron variant (e.g., a BA.1, BA.4/5, or XBB.1.5 Omicron variant)) and a tetravalent influenza RNA vaccine comprising 20 μg of RNA (e.g., 5 μg of an RNA encoding an HA protein of an H1N1 influenza strain, 5 μg of an RNA encoding an HA protein of an H3N2 influenza strain, 5 μg of an RNA encoding an HA protein of a B/Victoria influenza lineage, and 5 μg of an RNA encoding an HA protein of a B/Yamagata influenza lineage).

In some embodiments, a dose comprises a monovalent or bivalent SARS-CoV-2 RNA vaccine comprising 20 μg of total RNA (e.g., 20 μg of an RNA encoding a SARS-CoV-2 S protein of an Omicron variant (e.g., a BA.1, BA.4/5, or XBB.1.5 variant), or 10 μg of an RNA encoding a SARS-CoV-2 S protein of a Wuhan strain and 10 μg of an RNA encoding a SARS-CoV-2 S protein of an Omicron variant (e.g., a BA.1, BA.4/5, or XBB.1.5 Omicron variant)) and a tetravalent influenza RNA vaccine comprising 10 μg of total RNA (e.g., 2.5 μg of an RNA encoding an HA protein of an H1N1 influenza strain, 2.5 μg of an RNA encoding an HA protein of an H3N2 influenza strain, 2.5 μg of an RNA encoding an HA protein of a B/Victoria influenza lineage, and 2.5 μg of an RNA encoding an HA protein of a B/Yamagata influenza lineage).

In some embodiments, a dose comprises a monovalent or bivalent SARS-CoV-2 RNA vaccine comprising 30 μg of total RNA (e.g., 30 μg of an RNA encoding a SARS-CoV-2 S protein of an Omicron variant (e.g., a BA.1, BA.4/5, or XBB.1.5 variant), or 15 μg of an RNA encoding a SARS-CoV-2 S protein of a Wuhan strain and 15 μg of an RNA encoding a SARS-CoV-2 S protein of an Omicron variant (e.g., a BA.1, BA.4/5, or XBB.1.5 Omicron variant)) and a tetravalent influenza RNA vaccine comprising 30 μg of total RNA (e.g., 7.5 μg of an RNA encoding an HA protein of an H1N1 influenza strain, 7.5 μg of an RNA encoding an HA protein of an H3N2 influenza strain, 7.5 μg of an RNA encoding an HA protein of a B/Victoria influenza lineage, and 7.5 μg of an RNA encoding an HA protein of a B/Yamagata influenza lineage).

In some embodiments, a dose comprises a monovalent or bivalent SARS-CoV-2 RNA vaccine comprising 30 μg of total RNA (e.g., 30 μg of an RNA encoding a SARS-CoV-2 S protein of an Omicron variant (e.g., a BA.1, BA.4/5, or XBB.1.5 variant), or 15 μg of an RNA encoding a SARS-CoV-2 S protein of a Wuhan strain and 15 μg of an RNA encoding a SARS-CoV-2 S protein of an Omicron variant (e.g., a BA.1, BA.4/5, or XBB.1.5 Omicron variant)) and a tetravalent influenza RNA vaccine comprising 60 μg of total RNA (e.g., 15 μg of an RNA encoding an HA protein of an H1N1 influenza strain, 15 μg of an RNA encoding an HA protein of an H3N2 influenza strain, 15 μg of an RNA encoding an HA protein of a B/Victoria influenza lineage, and 15 μg of an RNA encoding an HA protein of a B/Yamagata influenza lineage).

In some embodiments, a dose comprises a monovalent or bivalent SARS-CoV-2 RNA vaccine comprising 60 μg of total RNA (e.g., 60 μg of an RNA encoding a SARS-CoV-2 S protein of an Omicron variant (e.g., a BA.1, BA.4/5, or XBB.1.5 variant), or 30 μg of an RNA encoding a SARS-CoV-2 S protein of a Wuhan strain and 30 μg of an RNA encoding a SARS-CoV-2 S protein of an Omicron variant (e.g., a BA.1, BA.4/5, or XBB.1.5 Omicron variant)) and a tetravalent influenza RNA vaccine comprising 30 μg of total RNA (e.g., 7.5 μg of an RNA encoding an HA protein of an H1N1 influenza strain, 7.5 μg of an RNA encoding an HA protein of an H3N2 influenza strain, 7.5 μg of an RNA encoding an HA protein of a B/Victoria influenza lineage, and 7.5 μg of an RNA encoding an HA protein of a B/Yamagata influenza lineage).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) or a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 90, 7.5 μg of an RNA encoding an HA protein of an H3N2 influenza strain and comprising a sequence that is at least 85% identical to SEQ ID NO: 95, 7.5 μg of an RNA encoding an HA protein of a B/Victoria influenza lineage and comprising a sequence that is at least 85% identical to SEQ ID NO: 100, and 7.5 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 60 μg of total RNA (e.g., comprising 15 μg of an RNA encoding an HA protein of an H1N1 influenza strain and comprising a sequence that is at least 85% identical to SEQ ID NO: 90, 15 μg of an RNA encoding an HA protein of an H3N2 influenza strain and comprising a sequence that is at least 85% identical to SEQ ID NO: 95, 15 μg of an RNA encoding an HA protein of a B/Victoria influenza lineage and comprising a sequence that is at least 85% identical to SEQ ID NO: 100, and 15 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 30 μg of an RNA encoding a SARS-CoV-2 S protein of a Wuhan strain comprising a sequence that is at least 85% identical to SEQ ID NO: 7 and 30 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 90, 7.5 μg of an RNA encoding an HA protein of an H3N2 influenza strain and comprising a sequence that is at least 85% identical to SEQ ID NO: 95, 7.5 μg of an RNA encoding an HA protein of a B/Victoria influenza lineage and comprising a sequence that is at least 85% identical to SEQ ID NO: 100, and 7.5 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising about 30 μg of total RNA (e.g., 7.5 μg of an RNA encoding an HA protein of an H1N1 influenza strain and comprising a sequence that is at least 85% identical to SEQ ID NO: 92, 7.5 μg of an RNA encoding an HA protein of an H3N2 influenza strain and comprising a sequence that is at least 85% identical to SEQ ID NO: 97, 7.5 μg of an RNA encoding an HA protein of a B/Victoria influenza lineage and comprising a sequence that is at least 85% identical to SEQ ID NO: 102, and 7.5 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising about 60 μg of total RNA (e.g., 15 μg of an RNA encoding an HA protein of an H1N1 influenza strain and comprising a sequence that is at least 85% identical to SEQ ID NO: 92, 15 μg of an RNA encoding an HA protein of an H3N2 influenza strain and comprising a sequence that is at least 85% identical to SEQ ID NO: 97, 15 μg of an RNA encoding an HA protein of a B/Victoria influenza lineage and comprising a sequence that is at least 85% identical to SEQ ID NO: 102, and 15 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 30 μg of an RNA encoding a SARS-CoV-2 S protein of a Wuhan strain and comprising a sequence that is at least 85% identical to SEQ ID NO: 9 and 30 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 92, 7.5 μg of an RNA encoding an HA protein of an H3N2 influenza strain and comprising a sequence that is at least 85% identical to SEQ ID NO: 97, 7.5 μg of an RNA encoding an HA protein of a B/Victoria influenza lineage and comprising a sequence that is at least 85% identical to SEQ ID NO: 102, and 7.5 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 132, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising about 30 μg of total RNA (e.g., 7.5 μg of an RNA encoding an HA protein of an H1N1 influenza strain and comprising a sequence that is at least 85% identical to SEQ ID NO: 94, 7.5 μg of an RNA encoding an HA protein of an H3N2 influenza strain and comprising a sequence that is at least 85% identical to SEQ ID NO: 99, 7.5 μg of an RNA encoding an HA protein of a B/Victoria influenza lineage and comprising a sequence that is at least 85% identical to SEQ ID NO: 104, and 7.5 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 132, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 60 μg of total RNA (e.g., 15 μg of an RNA encoding an HA protein of an H1N1 influenza strain and comprising a sequence that is at least 85% identical to SEQ ID NO: 94, 15 μg of an RNA encoding an HA protein of an H3N2 influenza strain and comprising a sequence that is at least 85% identical to SEQ ID NO: 99, 15 μg of an RNA encoding an HA protein of a B/Victoria influenza lineage and comprising a sequence that is at least 85% identical to SEQ ID NO: 104, and 15 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 132, or (b) a bivalent SARS-CoV-2 RNA vaccine comprising 30 μg of an RNA encoding a SARS-CoV-2 S protein of a Wuhan strain and comprising a sequence that is at least 85% identical to SEQ ID NO: 20 and 30 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 94, 7.5 μg of an RNA encoding an HA protein of an H3N2 influenza strain and comprising a sequence that is at least 85% identical to SEQ ID NO: 99, 7.5 μg of an RNA encoding an HA protein of a B/Victoria influenza lineage and comprising a sequence that is at least 85% identical to SEQ ID NO: 104, and 7.5 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 90, 7.5 μg of an RNA encoding an HA protein of an H3N2 influenza strain and comprising a sequence that is at least 85% identical to SEQ ID NO: 80, 7.5 μg of an RNA encoding an HA protein of a B/Victoria influenza lineage and comprising a sequence that is at least 85% identical to SEQ ID NO: 85, and 7.5 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 60 μg of total RNA (e.g., 15 μg of an RNA encoding an HA protein of an H1N1 influenza strain and comprising a sequence that is at least 85% identical to SEQ ID NO: 90, 15 μg of an RNA encoding an HA protein of an H3N2 influenza strain and comprising a sequence that is at least 85% identical to SEQ ID NO: 80, 15 μg of an RNA encoding an HA protein of a B/Victoria influenza lineage and comprising a sequence that is at least 85% identical to SEQ ID NO: 85, and 15 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 30 μg of an RNA encoding a SARS-CoV-2 S protein of a Wuhan strain comprising a sequence that is at least 85% identical to SEQ ID NO: 7 and 30 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 90, 7.5 μg of an RNA encoding an HA protein of an H3N2 influenza strain and comprising a sequence that is at least 85% identical to SEQ ID NO: 80, 7.5 μg of an RNA encoding an HA protein of a B/Victoria influenza lineage and comprising a sequence that is at least 85% identical to SEQ ID NO: 85, and 7.5 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 92, 7.5 μg of an RNA encoding an HA protein of an H3N2 influenza strain and comprising a sequence that is at least 85% identical to SEQ ID NO: 82, 7.5 μg of an RNA encoding an HA protein of a B/Victoria influenza lineage and comprising a sequence that is at least 85% identical to SEQ ID NO: 87, and 7.5 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 60 μg of total RNA (e.g., 15 μg of an RNA encoding an HA protein of an H1N1 influenza strain and comprising a sequence that is at least 85% identical to SEQ ID NO: 92, 15 μg of an RNA encoding an HA protein of an H3N2 influenza strain and comprising a sequence that is at least 85% identical to SEQ ID NO: 82, 15 μg of an RNA encoding an HA protein of a B/Victoria influenza lineage and comprising a sequence that is at least 85% identical to SEQ ID NO: 87, and 15 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 30 μg of an RNA encoding a SARS-CoV-2 S protein of a Wuhan strain and comprising a sequence that is at least 85% identical to SEQ ID NO: 9 and 30 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 92, 7.5 μg of an RNA encoding an HA protein of an H3N2 influenza strain and comprising a sequence that is at least 85% identical to SEQ ID NO: 82, 7.5 μg of an RNA encoding an HA protein of a B/Victoria influenza lineage and comprising a sequence that is at least 85% identical to SEQ ID NO: 87, and 7.5 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 131, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 94, 7.5 μg of an RNA encoding an HA protein of an H3N2 influenza strain and comprising a sequence that is at least 85% identical to SEQ ID NO: 84, 7.5 μg of an RNA encoding an HA protein of a B/Victoria influenza lineage and comprising a sequence that is at least 85% identical to SEQ ID NO: 89, and 7.5 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 131, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 60 μg of total RNA (e.g., 15 μg of an RNA encoding an HA protein of an H1N1 influenza strain and comprising a sequence that is at least 85% identical to SEQ ID NO: 94, 15 μg of an RNA encoding an HA protein of an H3N2 influenza strain and comprising a sequence that is at least 85% identical to SEQ ID NO: 84, 15 μg of an RNA encoding an HA protein of a B/Victoria influenza lineage and comprising a sequence that is at least 85% identical to SEQ ID NO: 104, and 15 μg of an RNA encoding an HA protein of a B/Yamagata influenza lineage and comprising a sequence that is at least 85% identical to SEQ ID NO: 89).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 131, or (b) a bivalent SARS-CoV-2 RNA vaccine comprising 30 μg of an RNA encoding a SARS-CoV-2 S protein of a Wuhan strain and comprising a sequence that is at least 85% identical to SEQ ID NO: 20 and 30 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 30 μg of total RNA (e.g., 7.5 μg of an RNA encoding an HA protein of an H1N1 influenza strain and comprising a sequence that is at least 85% identical to SEQ ID NO: 94, 7.5 μg of an RNA encoding an HA protein of an H3N2 influenza strain and comprising a sequence that is at least 85% identical to SEQ ID NO: 84, 7.5 μg of an RNA encoding an HA protein of a B/Victoria influenza lineage and comprising a sequence that is at least 85% identical to SEQ ID NO: 89, and 7.5 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) or a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 45 μg of total RNA (e.g., 11.25 μg of an RNA encoding an HA protein of an H1N1 influenza strain and comprising a sequence that is at least 85% identical to SEQ ID NO: 90, 11.25 μg of an RNA encoding an HA protein of an H3N2 influenza strain and comprising a sequence that is at least 85% identical to SEQ ID NO: 95, 11.25 μg of an RNA encoding an HA protein of a B/Victoria influenza lineage and comprising a sequence that is at least 85% identical to SEQ ID NO: 100, and 11.25 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising about 45 μg of total RNA (e.g., 11.25 μg of an RNA encoding an HA protein of an H1N1 influenza strain and comprising a sequence that is at least 85% identical to SEQ ID NO: 92, 11.25 μg of an RNA encoding an HA protein of an H3N2 influenza strain and comprising a sequence that is at least 85% identical to SEQ ID NO: 97, 11.25 μg of an RNA encoding an HA protein of a B/Victoria influenza lineage and comprising a sequence that is at least 85% identical to SEQ ID NO: 102, and 11.25 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 132, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising about 45 μg of total RNA (e.g., 11.25 μg of an RNA encoding an HA protein of an H1N1 influenza strain and comprising a sequence that is at least 85% identical to SEQ ID NO: 94, 11.25 μg of an RNA encoding an HA protein of an H3N2 influenza strain and comprising a sequence that is at least 85% identical to SEQ ID NO: 99, 11.25 μg of an RNA encoding an HA protein of a B/Victoria influenza lineage and comprising a sequence that is at least 85% identical to SEQ ID NO: 104, and 11.25 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 45 μg of total RNA (e.g., 11.25 ug of an RNA encoding an HA protein of an H1N1 influenza strain and comprising a sequence that is at least 85% identical to SEQ ID NO: 90, 11.25 μg of an RNA encoding an HA protein of an H3N2 influenza strain and comprising a sequence that is at least 85% identical to SEQ ID NO: 80, 11.25 μg of an RNA encoding an HA protein of a B/Victoria influenza lineage and comprising a sequence that is at least 85% identical to SEQ ID NO: 85, and 11.25 ug of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 45 μg of total RNA (e.g., 11.25 μg of an RNA encoding an HA protein of an H1N1 influenza strain and comprising a sequence that is at least 85% identical to SEQ ID NO: 92, 11.25 μg of an RNA encoding an HA protein of an H3N2 influenza strain and comprising a sequence that is at least 85% identical to SEQ ID NO: 82, 11.25 μg of an RNA encoding an HA protein of a B/Victoria influenza lineage and comprising a sequence that is at least 85% identical to SEQ ID NO: 87, and 11.25 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 131, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 ug of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 45 μg of total RNA (e.g., 11.25 μg of an RNA encoding an HA protein of an H1N1 influenza strain and comprising a sequence that is at least 85% identical to SEQ ID NO: 94, 11.25 μg of an RNA encoding an HA protein of an H3N2 influenza strain and comprising a sequence that is at least 85% identical to SEQ ID NO: 84, 11.25 μg of an RNA encoding an HA protein of a B/Victoria influenza lineage and comprising a sequence that is at least 85% identical to SEQ ID NO: 89, and 11.25 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) or a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 60 μg of total RNA (e.g., 5 μg of an RNA encoding an HA protein of an H1N1 influenza strain and comprising a sequence that is at least 85% identical to SEQ ID NO: 90, 5 μg of an RNA encoding an HA protein of an H3N2 influenza strain and comprising a sequence that is at least 85% identical to SEQ ID NO: 95, 25 μg of an RNA encoding an HA protein of a B/Victoria influenza lineage and comprising a sequence that is at least 85% identical to SEQ ID NO: 100, and 25 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 60 μg of total RNA (e.g., 5 μg of an RNA encoding an HA protein of an H1N1 influenza strain and comprising a sequence that is at least 85% identical to SEQ ID NO: 92, 5 μg of an RNA encoding an HA protein of an H3N2 influenza strain and comprising a sequence that is at least 85% identical to SEQ ID NO: 97, 25 μg of an RNA encoding an HA protein of a B/Victoria influenza lineage and comprising a sequence that is at least 85% identical to SEQ ID NO: 102, and 25 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 132, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 60 μg of total RNA (e.g., 5 μg of an RNA encoding an HA protein of an H1N1 influenza strain and comprising a sequence that is at least 85% identical to SEQ ID NO: 94, 5 μg of an RNA encoding an HA protein of an H3N2 influenza strain and comprising a sequence that is at least 85% identical to SEQ ID NO: 99, 25 μg of an RNA encoding an HA protein of a B/Victoria influenza lineage and comprising a sequence that is at least 85% identical to SEQ ID NO: 104, and 25 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 60 μg of total RNA (e.g., 5 μg of an RNA encoding an HA protein of an H1N1 influenza strain and comprising a sequence that is at least 85% identical to SEQ ID NO: 90, 5 μg of an RNA encoding an HA protein of an H3N2 influenza strain and comprising a sequence that is at least 85% identical to SEQ ID NO: 80, 25 μg of an RNA encoding an HA protein of a B/Victoria influenza lineage and comprising a sequence that is at least 85% identical to SEQ ID NO: 85, and 25 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 60 μg of total RNA (e.g., 5 μg of an RNA encoding an HA protein of an H1N1 influenza strain and comprising a sequence that is at least 85% identical to SEQ ID NO: 92, 5 μg of an RNA encoding an HA protein of an H3N2 influenza strain and comprising a sequence that is at least 85% identical to SEQ ID NO: 82, 25 μg of an RNA encoding an HA protein of a B/Victoria influenza lineage and comprising a sequence that is at least 85% identical to SEQ ID NO: 87, and 25 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 131, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 60 μg of total RNA (e.g., 5 μg of an RNA encoding an HA protein of an H1N1 influenza strain and comprising a sequence that is at least 85% identical to SEQ ID NO: 94, 5 μg of an RNA encoding an HA protein of an H3N2 influenza strain and comprising a sequence that is at least 85% identical to SEQ ID NO: 84, 25 μg of an RNA encoding an HA protein of a B/Victoria influenza lineage and comprising a sequence that is at least 85% identical to SEQ ID NO: 89, and 25 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) or a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 90, 2.5 μg of an RNA encoding an HA protein of an H3N2 influenza strain and comprising a sequence that is at least 85% identical to SEQ ID NO: 95, 12.5 μg of an RNA encoding an HA protein of a B/Victoria influenza lineage and comprising a sequence that is at least 85% identical to SEQ ID NO: 100, and 12.5 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 92, 2.5 μg of an RNA encoding an HA protein of an H3N2 influenza strain and comprising a sequence that is at least 85% identical to SEQ ID NO: 97, 12.5 μg of an RNA encoding an HA protein of a B/Victoria influenza lineage and comprising a sequence that is at least 85% identical to SEQ ID NO: 102, and 12.5 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 132, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 94, 2.5 μg of an RNA encoding an HA protein of an H3N2 influenza strain and comprising a sequence that is at least 85% identical to SEQ ID NO: 99, 12.5 μg of an RNA encoding an HA protein of a B/Victoria influenza lineage and comprising a sequence that is at least 85% identical to SEQ ID NO: 104, and 12.5 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 15 μg of an RNA encoding a SARS-CoV-2 S protein of a Wuhan strain comprising a sequence that is at least 85% identical to SEQ ID NO: 7 and 15 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 30 μg of total RNA (e.g., 2.5 ug of an RNA encoding an HA protein of an H1N1 influenza strain and comprising a sequence that is at least 85% identical to SEQ ID NO: 90, 2.5 μg of an RNA encoding an HA protein of an H3N2 influenza strain and comprising a sequence that is at least 85% identical to SEQ ID NO: 80, 12.5 μg of an RNA encoding an HA protein of a B/Victoria influenza lineage and comprising a sequence that is at least 85% identical to SEQ ID NO: 85, and 12.5 ug of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 9 and 15 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 92, 2.5 μg of an RNA encoding an HA protein of an H3N2 influenza strain and comprising a sequence that is at least 85% identical to SEQ ID NO: 82, 12.5 μg of an RNA encoding an HA protein of a B/Victoria influenza lineage and comprising a sequence that is at least 85% identical to SEQ ID NO: 87, and 12.5 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 30 μg of an RNA encoding a SARS-CoV-2 S protein of an XBB.1.5 variant comprising a sequence that is at least 85% identical to SEQ ID NO: 131, or (b) a bivalent SARS-CoV-2 RNA vaccine comprising 15 μg of an RNA encoding a SARS-CoV-2 S protein of a Wuhan strain and comprising a sequence that is at least 85% identical to SEQ ID NO: 20 and 15 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 94, 2.5 μg of an RNA encoding an HA protein of an H3N2 influenza strain and comprising a sequence that is at least 85% identical to SEQ ID NO: 84, 12.5 μg of an RNA encoding an HA protein of a B/Victoria influenza lineage and comprising a sequence that is at least 85% identical to SEQ ID NO: 89, and 12.5 μg of an RNA encoding an HA protein of a B/Yamagata influenza lineage and comprising a sequence that is at least 85% identical to SEQ ID NO: 109).

In some embodiments, a dose comprises (i) (a) 30 μg of a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 129, or (b) or a bivalent SARS-CoV-2 RNA vaccine comprising 30 μg of an RNA encoding a SARS-CoV-2 S protein of a Wuhan strain comprising a sequence that is at least 85% identical to SEQ ID NO: 7 and 30 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 90, 2.5 μg of an RNA encoding an HA protein of an H3N2 influenza strain and comprising a sequence that is at least 85% identical to SEQ ID NO: 95, 12.5 μg of an RNA encoding an HA protein of a B/Victoria influenza lineage and comprising a sequence that is at least 85% identical to SEQ ID NO: 100, and 12.5 μg of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 30 μg of an RNA encoding a SARS-CoV-2 S protein of a Wuhan strain and comprising a sequence that is at least 85% identical to SEQ ID NO: 9 and 30 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 92, 2.5 μg of an RNA encoding an HA protein of an H3N2 influenza strain and comprising a sequence that is at least 85% identical to SEQ ID NO: 97, 12.5 μg of an RNA encoding an HA protein of a B/Victoria influenza lineage and comprising a sequence that is at least 85% identical to SEQ ID NO: 102, and 12.5 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 132, or (b) a bivalent SARS-CoV-2 RNA vaccine comprising 30 μg of an RNA encoding a SARS-CoV-2 S protein of a Wuhan strain and comprising a sequence that is at least 85% identical to SEQ ID NO: 20 and 30 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 94, 2.5 μg of an RNA encoding an HA protein of an H3N2 influenza strain and comprising a sequence that is at least 85% identical to SEQ ID NO: 99, 12.5 μg of an RNA encoding an HA protein of a B/Victoria influenza lineage and comprising a sequence that is at least 85% identical to SEQ ID NO: 104, and 12.5 μg of an RNA encoding an HA protein of a B/Yamagata influenza lineage and comprising a sequence that is at least 85% identical to SEQ ID NO: 109).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 129, or (b) a bivalent SARS-CoV-2 RNA vaccine comprising 30 μg of an RNA encoding a SARS-CoV-2 S protein of a Wuhan strain comprising a sequence that is at least 85% identical to SEQ ID NO: 7 and 30 μg of an RNA encoding a SARS-CoV-2 S protein of a BA.4/5 Omicron variant comprising a sequence that is at least 85% identical to SEQ ID NO: 69) and (ii) a tetravalent influenza RNA vaccine comprising 30 μg of total RNA (e.g., 2.5 ug of an RNA encoding an HA protein of an H1N1 influenza strain and comprising a sequence that is at least 85% identical to SEQ ID NO: 90, 2.5 μg of an RNA encoding an HA protein of an H3N2 influenza strain and comprising a sequence that is at least 85% identical to SEQ ID NO: 80, 12.5 μg of an RNA encoding an HA protein of a B/Victoria influenza lineage and comprising a sequence that is at least 85% identical to SEQ ID NO: 85, and 12.5 ug of an RNA encoding an HA protein of a B/Yamagata influenza lineage and comprising a sequence that is at least 85% identical to SEQ ID NO: 105).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 130, or (b) a bivalent SARS-CoV-2 RNA vaccine comprising 30 μg of an RNA encoding a SARS-CoV-2 S protein of a Wuhan strain and comprising a sequence that is at least 85% identical to SEQ ID NO: 9 and 30 μg of an RNA encoding a SARS-CoV-2 S protein of a BA.4/5 Omicron variant and comprising a sequence that is at least 85% identical to SEQ ID NO: 70) and (ii) a tetravalent influenza RNA vaccine comprising 30 μg of total RNA (e.g., 2.5 μg of an RNA encoding an HA protein of an H1N1 influenza strain and comprising a sequence that is at least 85% identical to SEQ ID NO: 92, 2.5 μg of an RNA encoding an HA protein of an H3N2 influenza strain and comprising a sequence that is at least 85% identical to SEQ ID NO: 82, 12.5 μg of an RNA encoding an HA protein of a B/Victoria influenza lineage and comprising a sequence that is at least 85% identical to SEQ ID NO: 87, and 12.5 μg of an RNA encoding an HA protein of a B/Yamagata influenza lineage and comprising a sequence that is at least 85% identical to SEQ ID NO: 107).

In some embodiments, a dose comprises (i) (a) a monovalent SARS-CoV-2 RNA vaccine comprising 60 μg of an RNA encoding a SARS-CoV-2 S protein of an XBB.1.5 variant comprising a sequence that is at least 85% identical to SEQ ID NO: 131, or (b) a bivalent SARS-CoV-2 RNA vaccine comprising 30 μg of an RNA encoding a SARS-CoV-2 S protein of a Wuhan strain and comprising a sequence that is at least 85% identical to SEQ ID NO: 20 and 30 μg of an RNA encoding a SARS-CoV-2 S protein of a BA.4/5 Omicron variant and comprising a sequence that is at least 85% identical to SEQ ID NO: 72) and (ii) a tetravalent influenza RNA vaccine comprising 30 μg of RNA (e.g., 2.5 μg of an RNA encoding an HA protein of an H1N1 influenza strain and comprising a sequence that is at least 85% identical to SEQ ID NO: 94, 2.5 μg of an RNA encoding an HA protein of an H3N2 influenza strain and comprising a sequence that is at least 85% identical to SEQ ID NO: 84, 12.5 μg of an RNA encoding an HA protein of a B/Victoria influenza lineage and comprising a sequence that is at least 85% identical to SEQ ID NO: 89, and 12.5 μg of an RNA encoding an HA protein of a B/Yamagata influenza lineage and comprising a sequence that is at least 85% identical to SEQ ID NO: 109).

In some embodiments, a composition comprising one or more first RNAs, each comprising a nucleotide sequence encoding an antigenic polypeptide associated with a coronavirus and a composition comprising one or more second RNAs, each encoding an antigenic polypeptide associated with an influenza virus (e.g., a composition comprising an at least bivalent SARS-CoV-2 vaccine and an at least tetravalent influenza vaccine) is administered as a booster dose to a subject who has previously received a vaccine against coronavirus and/or influenza (e.g., a vaccine described herein). In some embodiments, such a booster dose is administered at least two months after a previous vaccination (e.g., at least two months after previous vaccination against SARS-CoV-2 or at least two months after previous vaccination against coronavirus). In some embodiments, such a booster dose is administered as part of a regular (e.g., annual) dosing regimen.

In some embodiments, a composition comprising one or more first RNAs, each comprising a nucleotide sequence encoding an antigenic polypeptide associated with a coronavirus, is administered in combination with a composition that comprises or delivers one or more antigenic polypeptides associated with an influenza virus. In some embodiments, the one or more first RNAs and the composition comprising or delivering one or more antigenic polypeptides associated with an influenza virus are administered separately (e.g., I.M. administered at different injection sites). In some embodiments, the composition comprising or delivering one or more antigenic polypeptides associated with an influenza virus comprises one or more RNAs, each encoding one or more antigenic polypeptides. In some embodiments, the composition comprising or delivering one or more antigenic polypeptides associated with an influenza virus is a commercially approved influenza vaccine. In some embodiments, the composition comprising or delivering one or more antigenic polypeptides associated with an influenza virus is an inactivated influenza virus (e.g., Fluzone®, Fluzone high-dose Quadrivalent®, Fluzone Quadrivalent®, Fluzone intradermal Quardivalent®, Fluzone quadrivalent southern Hemisphere®, Fluad®, Fluad Quadrivalent®, Afluria Quardivalent®, Fluarix Quadrivalent®, FluLaval Quadrivalent®, or Flucelvax Quadrivalent®), a recombinant influenza vaccine (e.g., Flublok Quadrivalent®), a live attenuated influenza vaccine (e.g., FluMist Quadrivalent®), an unadjuvanted influenza vaccine, an adjuvant influenza vaccine, or a subunit or split vaccine. In some embodiments, the composition comprising or delivering one or more antigenic polypeptides associated with an influenza virus is a nanoparticle vaccine, e.g., as described in Boyoglu-Bamum, Seyhan, et al. “Quadrivalent influenza nanoparticle vaccines induce broad protection,” Nature 592.7855 (2021): 623-628, the contents of which are incorporated by reference herein in their entirety.

In some embodiments, a composition comprising one or more second RNAs, each encoding an antigenic polypeptide associated with an influenza virus is administered in combination with a composition that comprises or delivers one or more antigenic polypeptides associated with a coronavirus. In some embodiments, the one or more second RNAs and the composition comprising or delivering one or more antigenic polypeptides associated with a coronavirus are administered separately (e.g., via I.M. injection at different injection sites). In some embodiments, the composition comprising or delivering one or more antigenic polypeptides associated with a coronavirus is a BNT162b2 vaccine (including, e.g., a variant adapted-BNT162b2, as described herein, an mRNA-1273 vaccine, an Ad26.CoV2.S vaccine, a ChAdxOx1 vaccine, an NVX-CoV2373 vaccine, a CvnCoV vaccine, a GAM-COVID0Vac vaccine, a CoronaVac vaccine, a BBIBP-CorV vaccine, an Ad5-nCOV vaccine, a zf2001 vaccine, a SCB-2019 vaccine, a JNJ 78436735 vaccine, or other approved mRNA or adenovector vaccines, etc.

In some embodiments, a composition comprising one or more first RNAs, each comprising a nucleotide sequence encoding an antigenic polypeptide associated with a coronavirus and a composition comprising one or more second RNAs, each encoding an antigenic polypeptide associated with an influenza virus (e.g., a composition comprising an at least bivalent SARS-CoV-2 vaccine and an at least tetravalent influenza vaccine) is administered as to a vaccine naïve subject (i.e., a patient who has not previously received a vaccine against SARS-CoV-2 and/or influenza). In such embodiments, the composition may be administered in two doses (e.g., two doses administered about 7 to about 28 days apart).

In some embodiments, a subsequent dose given to an individual (e.g., as part of a primary regimen or booster regimen) can have the same amount of RNA as previously given to the individual. In some embodiments, a subsequent dose given to an individual (e.g., as part of a primary regimen or booster regimen) can differ in the amount of RNA, as compared to the amount previously given to the individual. For example, in some embodiments, a subsequent dose can be higher or lower than the prior dose, for example, based on consideration of various factors, including, e.g., immunogenicity and/or reactogenicity induced by the prior dose, prevalence of the disease, etc. In some embodiments, a subsequent dose can be higher than a prior dose by at least 30%, at least 40%, at least 50%, at least 60%, at least 70%, at least 80%, at least 90%, or higher. In some embodiments, a subsequent dose can be higher than a prior dose by at least 1.5-fold, at least 2-fold, at least 2.5 fold, at least 3-fold, or higher. In some embodiments, a subsequent dose can be higher than a prior dose by at least 30%, at least 40%, at least 50%, at least 60%, at least 70%, at least 80%, at least 90%, or higher. In some embodiments, a subsequent dose can be lower than a prior dose by at least 10%, at least 20%, at least 30%, at least 40%, at least 50%, at least 60%, at least 70% or lower. In some embodiments, an amount the RNA described herein from 0.1 μg to 300 μg, 0.5 μg to 200 μg, or 1 μg to 100 μg, such as about 1 μg, about 2 μg, about 3 μg, about 10 μg, about 15 μg, about 20 μg, about 25 μg, about 30 μg, about 35 μg, about 40 μg, about 45 μg, about 50 μg, about 55 μg, about 60 μg, about 70 μg, about 80 μg, about 90 μg, or about 100 μg may be administered per dose (e.g., the total amount of RNA administered in a given dose).

In some embodiments, an amount of RNA described herein of 60 μg or lower, 55 μg or lower, 50 μg or lower, 45 μg or lower, 40 μg or lower, 35 μg or lower, 30 μg or lower, 25 μg or lower, 20 μg or lower, 15 μg or lower, 10 μg or lower, 5 μg or lower, 3 μg or lower, 2.5 μg or lower, or 1 μg or lower may be administered per dose (e.g., the total amount of RNA administered in a given dose).

In some embodiments, an amount of RNA described herein of at least 0.25 μg, at least 0.5 μg, at least 1 μg, at least 2 μg, at least 3 μg, at least 4 μg, at least 5 μg, at least 10 μg, at least 15 μg, at least 20 μg, at least 25 μg, at least 30 μg, at least 40 μg, at least 50 μg, or at least 60 μg may be administered per dose (e.g., the total amount of RNA administered in a given dose). In some embodiments, an amount of RNA described herein of at least 3 ug may be administered in at least one of given doses. In some embodiments, an amount of RNA described herein of at least 10 ug may be administered in at least one of given doses. In some embodiments, an amount of RNA described herein of at least 15 ug may be administered in at least one of given doses. In some embodiments, an amount of RNA described herein of at least 20 ug may be administered in at least one of given doses. In some embodiments, an amount of RNA described herein of at least 25 ug may be administered in at least one of given doses. In some embodiments, an amount of RNA described herein of at least 30 ug may be administered in at least one of given doses. In some embodiments, an amount of RNA described herein of at least 50 ug may be administered in at least one of given doses. In some embodiments, an amount of RNA described herein of at least 60 ug may be administered in at least one of given doses. In some embodiments, combinations of aforementioned amounts may be administered in a regimen comprising two or more doses (e.g., a prior dose and a subsequent dose can be of different amounts as described herein). In some embodiments, combinations of aforementioned amounts may be administered in a primary regimen and a booster regimen (e.g., different doses can be given in a primary regimen and a booster regimen).

In some embodiments, an amount of an RNA described herein of 0.25 μg to 60 μg, 0.5 μg to 55 μg, 1 μg to 50 μg, 5 μg to 40 μg, or 10 μg to 30 μg may be administered per dose (e.g., the total amount of RNA administered in a given dose). In some embodiments, an amount of RNA described herein of 3 μg to 30 μg may be administered in at least one of given doses. In some embodiments, an amount of RNA described herein of 3 μg to 20 μg may be administered in at least one of given doses. In some embodiments, an amount of RNA described herein of 3 μg to μg may be administered in at least one of given doses. In some embodiments, an amount of RNA described herein of 3 μg to 10 μg may be administered in at least one of given doses. In some embodiments, an amount of RNA described herein of 10 μg to 30 μg may be administered in at least one of given doses.

In some embodiments, a regimen administered to a subject may comprise a plurality of doses (e.g., at least two doses, at least three doses, or more). In some embodiments, a regimen administered to a subject may comprise a first dose and a second dose, which are given at least 2 weeks apart, at least 3 weeks apart, at least 4 weeks apart, or more. In some embodiments, such doses may be at least 1 month, at least 2 months, at least 3 months, at least 4 months, at least 5 months, at least 6 months, at least 7 months, at least 8 months, at least 9 months, at least 10 months, at least 11 months, at least 12 months, or more apart. In some embodiments, doses may be administered days apart, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more days apart. In some embodiments, doses may be administered about 1 to about 3 weeks apart, or about 1 to about 4 weeks apart, or about 1 to about 5 weeks apart, or about 1 to about 6 weeks apart, or about 1 to more than 6 weeks apart. In some embodiments, doses may be separated by a period of about 7 to about 60 days, such as for example about 14 to about 48 days, etc. In some embodiments, a minimum number of days between doses may be about 1, 2, 3, 4, 5, 6, 7, 8, 9, 10, 11, 12, 13, 14, 15, 16, 17, 18, 19, 20, 21 or more. In some embodiments, a maximum number of days between doses may be about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or fewer. In some embodiments, doses may be about 21 to about 28 days apart. In some embodiments, doses may be about 19 to about 42 days apart. In some embodiments, doses may be about 7 to about 28 days apart. In some embodiments, doses may be about 14 to about 24 days. In some embodiments, doses may be about 21 to about 42 days.

In some embodiments, a vaccination regimen comprises a first dose and a second dose. In some embodiments, a first dose and a second dose are administered by at least 21 days apart. In some embodiments, a first dose and a second dose are administered by at least 28 days apart.

In some embodiments, a vaccination regimen comprises a first dose and a second dose, wherein the amount of RNA administered in the first dose is the same as the amount of RNA administered in the second dose. In some embodiments, a vaccination regimen comprises a first dose and a second dose wherein the amount of RNA administered in the first dose differs from that administered in the second dose.

In some embodiments, a vaccination regimen comprises a first dose and a second dose, wherein the amount of RNA administered in the first dose is less than that administered in the second dose. In some embodiments, the amount of RNA administered in the first dose is 10%-90% of the second dose. In some embodiments, the amount of RNA administered in the first dose is 10%-50% of the second dose. In some embodiments, the amount of RNA administered in the first dose is 10%-20% of the second dose. In some embodiments, the first dose and the second dose are administered at least 2 weeks apart, including, at least 3 weeks apart, at least 4 weeks apart, at least 5 weeks apart, at least 6 weeks apart or longer. In some embodiments, the first dose and the second dose are administered at least 3 weeks apart.

In some embodiments, a first dose comprises less than about 30 ug of total RNA and a second dose comprises at least about 30 ug of total RNA. In some embodiments, a first dose comprises about 1 to less than about 30 ug of total RNA (e.g., about 0.1, about 1, about 3, about 5, about 10, about 15, about 20, about 25, or less than about 30 ug of total RNA) and a second dose comprises about 30 to about 100 ug of total RNA (e.g., about 30, about 40, about 50, or about 60 ug of total RNA). In some embodiments, a first dose comprises about 1 to about 20 ug of total RNA, about 1 to about 10 ug of total RNA, or about 1 to about 5 ug of total RNA and a second dose comprises about 30 to about 60 ug of total RNA.

In some embodiments, a first dose comprises about 1 to about 10 ug of total RNA (e.g., about 1, about 2, about 3, about 4, about 5, about 6, about 7, about 8, about 9, or about 10 ug of total RNA) and a second dose comprises about 30 to about 60 ug of total RNA (e.g., about 30, about 35, about 40, about 45, about 50, about 55, or about 60 ug of total RNA).

In some embodiments, a first dose comprises about 1 ug of total RNA and a second dose comprises about 30 ug of total RNA. In some embodiments, a first dose comprises about 3 ug of total RNA and a second dose comprises about 30 ug of total RNA. In some embodiments, a first dose comprises about 5 ug of total RNA and a second dose comprises about 30 ug of total RNA. In some embodiments, a first dose comprises about 10 ug of total RNA and a second dose comprises about 30 ug of total RNA. In some embodiments, a first dose comprises about 15 ug of total RNA and a second dose comprises about 30 ug of total RNA.

In some embodiments, a first dose comprises about 1 ug of total RNA and a second dose comprises about 60 ug of total RNA. In some embodiments, a first dose comprises about 3 ug of total RNA and a second dose comprises about 60 ug of total RNA. In some embodiments, a first dose comprises about 5 ug of total RNA and a second dose comprises about 60 ug of total RNA. In some embodiments, a first dose comprises about 6 ug of total RNA and a second dose comprises about 60 ug of total RNA. In some embodiments, a first dose comprises about 10 ug of total RNA and a second dose comprises about 60 ug of total RNA. In some embodiments, a first dose comprises about 15 ug of total RNA and a second dose comprises about 60 ug of total RNA. In some embodiments, a first dose comprises about 20 ug of total RNA and a second dose comprises about 60 ug of total RNA. In some embodiments, a first dose comprises about 25 ug of total RNA and a second dose comprises about 60 ug of total RNA. In some embodiments, a first dose comprises about 30 ug of total RNA and a second dose comprises about 60 ug of total RNA.

In some embodiments, a first dose comprises less than about 10 ug of total RNA and a second dose comprises at least about 10 ug of total RNA. In some embodiments, a first dose comprises about 0.1 to less than about 10 ug of total RNA (e.g., about 0.1, about 0.5, about 1, about 2, about 3, about 4, about 5, about 6, about 7, about 8, or less than about 10 ug of total RNA) and a second dose comprises about 10 to about 30 ug of total RNA (e.g., about 10, about 15, about 20, about 25, or about 30 ug of total RNA). In some embodiments, a first dose comprises about 0.1 to about 10 ug of total RNA, about 1 to about 5 ug of total RNA, or about 0.1 to about 3 ug of total RNA and a second dose comprises about 10 to about 30 ug of total RNA.

In some embodiments, a first dose comprises about 0.1 to about 5 ug of total RNA (e.g., about 0.1, about 0.5, about 1, about 2, about 3, about 4, about 5 ug of total RNA) and a second dose comprises about 10 to about 20 ug of total RNA (e.g., about 10, about 12, about 14, about 16, about 18, about 20 ug of RNA).

In some embodiments, a first dose comprises about 0.1 ug of total RNA and a second dose comprises about 10 ug of total RNA. In some embodiments, a first dose comprises about 0.3 ug of total RNA and a second dose comprises about 10 ug of total RNA. In some embodiments, a first dose comprises about 1 ug of total RNA and a second dose comprises about 10 ug of total RNA. In some embodiments, a first dose comprises about 3 ug of total RNA and a second dose comprises about 10 ug of total RNA.

In some embodiments, a first dose comprises less than about 3 ug of total RNA and a second dose comprises at least about 3 ug of total RNA. In some embodiments, a first dose comprises about 0.1 to less than about 3 ug of total RNA (e.g., about 0.1, about 0.2, about 0.3, about 0.5, about 0.6, about 0.7, about 0.8, about 0.9, about 1.0, about 1.5, about 2.0, or about 2.5 ug of total RNA) and a second dose comprises about 3 to about 10 ug of total RNA (e.g., about 3, about 4, about 5, about 6, or about 7, about 8, about 9, or about 10 ug of total RNA). In some embodiments, a first dose comprises about 0.1 to about 3 ug of total RNA, about 0.1 to about 1 ug of total RNA, or about 0.1 to about 0.5 ug of total RNA and a second dose comprises about 3 to about 10 ug of total RNA. In some embodiments, a first dose comprises about 0.1 to about 1.0 ug of total RNA (e.g., about 0.1, about 0.2, about 0.3, about 0.4, about 0.5, about 0.6, about 0.7, about 0.8, about 0.9, or about 1.0 ug of total RNA) and a second dose comprises about 1 to about 3 ug of total RNA (e.g., about 1.0, about 1.5, about 2.0, about 2.5, or about 3.0 ug of total RNA).

In some embodiments, a first dose comprises about 0.1 ug of total RNA and a second dose comprises about 3 ug of total RNA. In some embodiments, a first dose comprises about 0.3 ug of total RNA and a second dose comprises about 3 ug of total RNA. In some embodiments, a first dose comprises about 0.5 ug of total RNA and a second dose comprises about 3 ug of total RNA. In some embodiments, a first dose comprises about 1 ug of total RNA and a second dose comprises about 3 ug of total RNA.

In some embodiments, a vaccination regimen comprises a first dose and a second dose, wherein the amount of RNA administered in the first dose is greater than that administered in the second dose. In some embodiments, the amount of RNA administered in the second dose is 10%-90% of the first dose. In some embodiments, the amount of RNA administered in the second dose is 10%-50% of the first dose. In some embodiments, the amount of RNA administered in the second dose is 10%-20% of the first dose. In some embodiments, the first dose and the second dose are administered at least 2 weeks apart, including, at least 3 weeks apart, at least 4 weeks apart, at least 5 weeks apart, at least 6 weeks apart or longer. In some embodiments, the first dose and the second dose are administered at least 3 weeks apart.

In some embodiments, a first dose comprises at least about 30 ug of total RNA and a second dose comprises less than about 30 ug of total RNA. In some embodiments, a first dose comprises about 30 to about 100 ug of total RNA (e.g., about 30, about 40, about 50, or about 60 ug of total RNA) and a second dose comprises about 1 to about 30 ug of total RNA (e.g., about 0.1, about 1, about 3, about 5, about 10, about 15, about 20, about 25, or about 30 ug of total RNA). In some embodiments, a second dose comprises about 1 to about 20 ug of total RNA, about 1 to about 10 ug of total RNA, or about 1 to 5 ug of total RNA. In some embodiments, a first dose comprises about 30 to about 60 ug of total RNA and a second dose comprises about 1 to about 20 ug of total RNA, about 1 to about 10 ug of total RNA, or about 0.1 to about 3 ug of total RNA.

In some embodiments, a first dose comprises about 30 to about 60 ug of total RNA (e.g., about 30, about 35, about 40, about 45, about 50, about 55, or about 60 ug of total RNA) and a second dose comprises about 1 to about 10 ug of total RNA (e.g., about 1, about 2, about 3, about 4, about 5, about 6, about 7, about 8, about 9, or about 10 ug of RNA).

In some embodiments, a first dose comprises about 30 ug of total RNA and a second dose comprises about 1 ug of total RNA. In some embodiments, a first dose comprises about 30 ug of total RNA and a second dose comprises about 3 ug of total RNA. In some embodiments, a first dose comprises about 30 ug of total RNA and a second dose comprises about 5 ug of total RNA. In some embodiments, a first dose comprises about 30 ug of total RNA and a second dose comprises about 10 ug of total RNA. In some embodiments, a first dose comprises about 30 ug of total RNA and a second dose comprises about 15 ug of total RNA.

In some embodiments, a first dose comprises about 60 ug of total RNA and a second dose comprises about 1 ug of total RNA. In some embodiments, a first dose comprises about 60 ug of total RNA and a second dose comprises about 3 ug of total RNA. In some embodiments, a first dose comprises about 60 ug of total RNA and a second dose comprises about 5 ug of total RNA. In some embodiments, a first dose comprises about 60 ug of total RNA and a second dose comprises about 6 ug of total RNA. In some embodiments, a first dose comprises about 60 ug of total RNA and a second dose comprises about 10 ug of total RNA. In some embodiments, a first dose comprises about 60 ug of total RNA and a second dose comprises about 15 ug of total RNA. In some embodiments, a first dose comprises about 60 ug of total RNA and a second dose comprises about 20 ug of total RNA. In some embodiments, a first dose comprises about 60 ug of total RNA and a second dose comprises about 25 ug of total RNA. In some embodiments, a first dose comprises about 60 ug of total RNA and a second dose comprises about 30 ug of total RNA.

In some embodiments, a first dose comprises at least about 10 ug of total RNA and a second dose comprises less than about 10 ug of total RNA. In some embodiments, a first dose comprises about 10 to about 30 ug of total RNA (e.g., about 10, about 15, about 20, about 25, or about 30 ug of total RNA) and a second dose comprises about 0.1 to less than about 10 ug of total RNA (e.g., about 0.1, about 0.5, about 1, about 2, about 3, about 4, about 5, about 6, about 7, about 8, or less than about 10 ug of total RNA). In some embodiments, a first dose comprises about 10 to about 30 ug of total RNA, or about 0.1 to about 3 ug of total RNA and a second dose comprises about 1 to about 10 ug of total RNA, or about 1 to about 5 ug of total RNA.

In some embodiments, a first dose comprises about 10 to about 20 ug of total RNA (e.g., about 10, about 12, about 14, about 16, about 18, about 20 ug of total RNA) and a second dose comprises about 0.1 to about 5 ug of total RNA (e.g., about 0.1, about 0.5, about 1, about 2, about 3, about 4, or about 5 ug of total RNA).

In some embodiments, a first dose comprises about 10 ug of total RNA and a second dose comprises about 0.1 ug of total RNA. In some embodiments, a first dose comprises about 10 ug of total RNA and a second dose comprises about 0.3 ug of total RNA. In some embodiments, a first dose comprises about 10 ug of total RNA and a second dose comprises about 1 ug of total RNA. In some embodiments, a first dose comprises about 10 ug of total RNA and a second dose comprises about 3 ug of total RNA.

In some embodiments, a first dose comprises at least about 3 ug of total RNA and a second dose comprises less than about 3 ug of total RNA. In some embodiments, a first dose comprises about 3 to about 10 ug of total RNA (e.g., about 3, about 4, about 5, about 6, or about 7, about 8, about 9, or about 10 ug of total RNA) and a second dose comprises 0.1 to less than about 3 ug of total RNA (e.g., about 0.1, about 0.2, about 0.3, about 0.5, about 0.6, about 0.7, about 0.8, about 0.9, about 1.0, about 1.5 about 2.0, or about 2.5 ug of total RNA). In some embodiments, a first dose comprises about 3 to about 10 ug of total RNA and a second dose comprises about 0.1 to about 3 ug of total RNA, about 0.1 to about 1 ug of total RNA, or about 0.1 to about 0.5 ug of total RNA.

In some embodiments, a first dose comprises about 1 to about 3 ug of total RNA (e.g., about 1, about 1.5, about 2.0, about 2.5, or about 3.0 ug of total RNA) and a second dose comprises about 0.1 to 0.3 ug of total RNA (e.g., about 0.1, about 0.2, about 0.3, about 0.4, about 0.5, about 0.6, about 0.7, about 0.8, about 0.9, or about 1.0 ug of total RNA).

In some embodiments, a first dose comprises about 3 ug of total RNA and a second dose comprises about 0.1 ug of total RNA. In some embodiments, a first dose comprises about 3 ug of total RNA and a second dose comprises about 0.3 ug of total RNA. In some embodiments, a first dose comprises about 3 ug of total RNA and a second dose comprises about 0.6 ug of total RNA. In some embodiments, a first dose comprises about 3 ug of total RNA and a second dose comprises about 1 ug of total RNA.

In some embodiments, a vaccination regimen comprises at least two doses, including, e.g., at least three doses, at least four doses or more. In some embodiments, a vaccination regimen comprises three doses. In some embodiments, the time interval between the first dose and the second dose can be the same as the time interval between the second dose and the third dose. In some embodiments, the time interval between the first dose and the second dose can be longer than the time interval between the second dose and the third dose, e.g., by days or weeks (including, e.g., at least 3 days, at least 4 days, at least 5 days, at least 6 days, at least 1 week, at least 2 weeks, at least 3 weeks, at least 4 weeks, at least 5 weeks, at least 6 weeks, or longer). In some embodiments, the time interval between the first dose and the second dose can be shorter than the time interval between the second dose and the third dose, e.g., by days or weeks (including, e.g., at least 3 days, at least 4 days, at least 5 days, at least 6 days, at least 1 week, at least 2 weeks, at least 3 weeks, at least 4 weeks, at least 5 weeks, at least 6 weeks, or longer). In some embodiments, the time interval between the first dose and the second dose can be shorter than the time interval between the second dose and the third dose, e.g., by at least 1 month (including, e.g., at least 2 months, at least 3 months, at least 4 months, at least 5 months, at least 6 months, at least 7 months, at least 8 months, at least 9 months, at least 10 months, at least 11 months, at least 12 months, or longer).

In some embodiments, a last dose of a primary regimen and a first dose of a booster regimen are given at least 2 months, at least 3 months, at least 4 months, at least 5 months, at least 6 months, at least 7 months, at least 8 months, at least 9 months, at least 10 months, at least 11 months, at least 12 months, or more apart. In some embodiments, a primary regimen may comprises two doses. In some embodiments, a primary regimen may comprises three doses.

In some embodiments, a first dose and a second dose (and/or other subsequent dose) may be administered by intramuscular injection. In some embodiments, a first dose and a second dose (and/or other subsequent dose) may be administered in the deltoid muscle. In some embodiments, a first dose and a second dose (and/or other subsequent dose) may be administered in the same arm.

In some embodiments, an mRNA composition described herein is administered (e.g., by intramuscular injection) as a series of two doses (e.g., 0.3 mL each) 21 days apart. In some embodiments, an mRNA composition described herein is administered (e.g., by intramuscular injection) as a series of two doses (e.g., 0.2 mL each) 21 days apart. In some embodiments, an mRNA composition described herein is administered (e.g., by intramuscular injection) as a series of three doses (e.g., 0.3 mL or lower including, e.g., 0.2 mL), wherein doses are given at least 3 weeks apart. In some embodiments, the first and second doses may be administered 3 weeks apart, while the second and third doses may be administered at a longer time interval than that between the first and the second doses, e.g., at least 4 weeks apart or longer (including, at least 5 weeks, at least 6 weeks, at least 7 weeks, at least 8 weeks, at least 9 weeks, or longer). In some embodiments, each dose comprises about 60 ug of total RNA. In some embodiments, each dose comprises about 50 ug of total RNA. In some embodiments, each dose comprises about 30 ug of total RNA. In some embodiments, each dose comprises about 25 ug of total RNA. In some embodiments, each dose comprises about 20 ug of total RNAI. In some embodiments, each dose comprises about 15 ug of total RNA. In some embodiments, each dose comprises about 10 ug of total RNA. In some embodiments, each dose comprise about 3 ug of total RNA.

In some embodiments, at least one dose given in a vaccination regimen (e.g., a primary vaccination regimen and/or a booster vaccination regimen) comprises about 60 ug of total RNA. In some embodiments, at least one dose given in a vaccination regimen (e.g., a primary vaccination regimen and/or a booster vaccination regimen) comprises about 50 ug of total RNA. In some embodiments, at least one dose given in a vaccination regimen (e.g., a primary vaccination regimen and/or a booster vaccination regimen) comprises about 30 ug of total RNA. In some embodiments, at least one dose given in a vaccination regimen (e.g., a primary vaccination regimen and/or a booster vaccination regimen) comprises about 25 ug of total RNA. In some embodiments, at least one dose given in a vaccination regimen (e.g., a primary vaccination regimen and/or a booster vaccination regimen) comprises about 20 ug of total RNA. In some embodiments, at least one dose given in a vaccination regimen (e.g., a primary vaccination regimen and/or a booster vaccination regimen) comprises about 15 ug of total RNA. In some embodiments, at least one dose given in a vaccination regimen (e.g., a primary vaccination regimen and/or a booster vaccination regimen) comprises about 10 ug of total RNA. In some embodiments, at least one dose given in a vaccination regimen (e.g., a primary vaccination regimen and/or a booster vaccination regimen) comprises about 3 ug of total RNA.

In one embodiment, an amount of RNA described herein of about 60 μg is administered per dose. In one embodiment, an amount of RNA described herein of about 50 μg is administered per dose. In one embodiment, an amount of RNA described herein of about 30 μg is administered per dose. In one embodiment, an amount of RNA described herein of about 25 μg is administered per dose. In one embodiment, an amount of RNA described herein of about 20 μg is administered per dose. In one embodiment, an amount of RNA described herein of about 15 μg is administered per dose. In one embodiment, an amount of RNA described herein of about 10 μg is administered per dose. In one embodiment, an amount of RNA described herein of about 5 μg is administered per dose. In one embodiment, an amount of RNA described herein of about 3 μg is administered per dose. In one embodiment, at least two of such doses are administered. For example, a second dose may be administered about 21 days following administration of the first dose.

In some embodiments, the efficacy of an RNA vaccine described herein (e.g., administered in two doses, wherein a second dose may be administered about 21 days following administration of the first dose, and administered, for example, in an amount of about 30 μg per dose) is at least 70%, at least 80%, at least 90, or at least 95% beginning 7 days after administration of the second dose (e.g., beginning 28 days after administration of the first dose if a second dose is administered 21 days following administration of the first dose). In some embodiments, such efficacy is observed in populations of age of at least 50, at least 55, at least 60, at least 65, at least 70, or older. In some embodiments, the efficacy of an RNA vaccine described herein (e.g., administered in two doses, wherein a second dose may be administered about 21 days following administration of the first dose, and administered, for example, in an amount of about 30 μg per dose) beginning 7 days after administration of the second dose (e.g., beginning 28 days after administration of the first dose if a second dose is administered 21 days following administration of the first dose) in populations of age of at least 65, such as 65 to 80, 65 to 75, or 65 to 70, is at least 90%, at least 91%, at least 92%, at least 93%, at least 94%, or at least 95%. Such efficacy may be observed over time periods of up to 1 month, 2 months, 3 months, 6 months or even longer.

In one embodiment, vaccine efficacy is defined as the percent reduction in the number of subjects with evidence of infection (vaccinated subjects vs. non-vaccinated subjects).

In one embodiment, efficacy is assessed through surveillance for potential cases of COVID-19 and/or influenza. If, at any time, a patient develops acute respiratory illness, for the purposes herein, the patient can be considered to potentially have COVID-19 illness and/or influenza illness. The assessments can include a nasal (midturbinate) swab, which may be tested using a reverse transcription-polymerase chain reaction (RT-PCR) test to detect SARS-CoV-2 or influenza. In addition, clinical information and results from local standard-of-care tests can be assessed. In some embodiments, efficacy assessments may utilize a definition of SARS-CoV-2-related cases wherein:
Confirmed COVID-19: presence of at least 1 of the following symptoms and SARS-CoV-2 NAAT (nucleic acid amplification-based test) positive during, or within 4 days before or after, the symptomatic period: fever; new or increased cough; new or increased shortness of breath; chills; new or increased muscle pain; new loss of taste or smell; sore throat; diarrhea; vomiting.

Alternatively or additionally, in some embodiments, efficacy assessments may utilize a definition of SARS-CoV-2-related cases wherein one or more of the following additional symptoms defined by the CDC can be considered: fatigue; headache; nasal congestion or runny nose; nausea.

In some embodiments, efficacy assessments may utilize a definition of SARS-CoV-2-related severe cases
Confirmed severe COVID-19: confirmed COVID-19 and presence of at least 1 of the following: clinical signs at rest indicative of severe systemic illness (e.g., RR≥30 breaths per minute, HR≥125 beats per minute, SpO₂≤93% on room air at sea level, or PaO₂/FiO₂<300 mm Hg); respiratory failure (which can be defined as needing high-flow oxygen, noninvasive ventilation, mechanical ventilation, or ECMO); evidence of shock (e.g., SBP<90 mm Hg, DBP<60 mm Hg, or requiring vasopressors); significant acute renal, hepatic, or neurologic dysfunction; admission to an ICU; death.

Alternatively or additionally, in some embodiments a serological definition can be used for patients without clinical presentation of COVID-19: e.g., confirmed seroconversion to SARS-CoV-2 without confirmed COVID-19: e.g., positive N-binding antibody result in a patient with a prior negative N-binding antibody result.

In some embodiments, any or all of the following assays can be performed on serum samples: SARS-CoV-2 neutralization assay; S1-binding IgG level assay; RBD-binding IgG level assay; N-binding antibody assay.

In one embodiment, methods and agents described herein are administered to a paediatric population. In various embodiments, the paediatric population comprises or consists of subjects under 18 years, e.g., 5 to less than 18 years of age, 12 to less than 18 years of age, 16 to less than 18 years of age, 12 to less than 16 years of age, 5 to less than 12 years of age, or 6 months to less than 12 years of age. In various embodiments, the paediatric population comprises or consists of subjects under 5 years, e.g., 2 to less than 5 years of age, 12 to less than 24 months of age, 7 to less than 12 months of age, or less than 6 months of age. In some such embodiments, an mRNA composition described herein is administered to subjects of less than 2 years old, for example, 6 months to less than 2 years old. In some such embodiments, an mRNA composition described herein is administered to subjects of less than 6 months old, for example, 1 month to less than 4 months old. In some embodiments, a dosing regimen (e.g., doses and/or dosing schedule) for a paediatric population may vary for different age groups. For example, in some embodiments, a subject 6 months through 4 years of age may be administered according to a primary regimen comprising at least three doses, in which the initial two doses are administered at least 3 weeks (including, e.g., at least 4 weeks, at least 5 weeks, at least 6 weeks, or longer) apart followed by a third dose administered at least 8 weeks (including, e.g., at least 9 weeks, at least 10 weeks, at least 11 weeks, at least 12 weeks, or longer) after the second dose. In some such embodiments, at least one dose administered comprises 3 μg of total RNA (e.g., of one or more RNAs described herein). In some embodiments, a subject 5 years of age and older may be administered according to a primary regimen comprising at least two doses, in which the two doses are administered at least 3 weeks (including, e.g., at least 3 weeks, at least 4 weeks, at least 5 weeks, at least 6 weeks, or longer) apart. In some such embodiments, at least one dose administered comprises 10 μg of total RNA (e.g., of one or more RNAs described herein). In some embodiments, a subject 5 years of age and older who are immunocompromised (e.g., in some embodiments subjects who have undergone solid organ transplantation, or who are diagnosed with conditions that are considered to have an equivalent of immunocompromise) may be administered according to a primary regimen comprising at least three doses, in which the initial two doses are administered at least 3 weeks (including, e.g., at least 3 weeks, at least 4 weeks, at least 5 weeks, at least 6 weeks, or longer) apart, followed by a third dose administered at least 4 weeks (including, e.g., at least 5 weeks, at least 6 weeks, at least 7 weeks, at least 8 weeks, at least 9 weeks, at least 10 weeks, at least 11 weeks, at least 12 weeks, or longer) after the second dose.

In some embodiments, an mRNA composition described herein is administered to subjects of age 12 or older and each dose comprises about 30 ug of total RNA. In some embodiments, an mRNA composition described herein is administered to subjects of age 12 or older (including, e.g., age 18 or older) and each dose comprises greater than 30 ug of total RNA, including, e.g., 35 ug, 40 ug, 45 ug, 50 ug, 55 ug, 60 ug, 65 ug, 70 ug, or higher. In some such embodiments, an mRNA composition described herein is administered to subjects of age 12 or older and each dose comprises about 60 ug of total RNA. In some such embodiments, an mRNA composition described herein is administered to subjects of age 12 or older and each dose comprises about 50 ug of total RNA. In one embodiment, the paediatric population comprises or consists of subjects 12 to less than 18 years of age including subjects 16 to less than 18 years of age and/or subjects 12 to less than 16 years of age. In this embodiment, treatments may comprise 2 vaccinations 21 days apart, wherein, in one embodiment, the vaccine is administered in an amount of 30 μg RNA per dose, e.g., by intramuscular administration. In some embodiments, higher doses are administered to older pediatric patients and adults, e.g., to patients 12 years or older, compared to younger children or infants, e.g. 2 to less than 5 years old, 6 months to less than 2 years old, or less than 6 months old. In some embodiments, higher doses are administered to children who are 2 to less than 5 years old, as compared to toddlers and/or infants, e.g., who are 6 months to less than 2 years old, or less than 6 months old.

In one embodiment, the paediatric population comprises or consists of subjects 5 to less than 18 years of age including subjects 12 to less than 18 years of age and/or subjects 5 to less than 12 years of age. In this embodiment, treatments may comprise 2 vaccinations 21 days apart, wherein, in various embodiments, the vaccine is administered in an amount of 10 μg, 20 μg, or 30 μg of total RNA per dose, e.g., by intramuscular administration.

In some such embodiments, an mRNA composition described herein is administered to subjects of age 5 to 11 and each dose is about 10 μg per dose.

In some embodiments, each dose comprises about 5 ug of RNA encoding a SARS-CoV-2 S protein of a first variant and about 5 ug of RNA encoding a SARS-CoV-2 S protein of a second variant. In some embodiments, each dose comprises about 5 ug of RNA encoding a SARS-CoV-2 S protein of a Wuhan strain and about 5 ug of RNA encoding a SARS-CoV-2 S protein of an Omicron variant (e.g., a BA.1, BA.4/5, or XBB.1.5 Omicron variant). In some embodiments, each dose comprises about 5 ug of RNA encoding a SARS-CoV-2 S protein of a Wuhan strain (e.g., RNA comprising SEQ ID NO: 20) and about 5 ug of RNA encoding a SARS-CoV-2 S protein of a BA.1 Omicron variant (e.g., RNA comprising SEQ ID NO: 51). In some embodiments, each dose comprises about 5 ug of RNA encoding a SARS-CoV-2 S protein of a Wuhan strain (e.g., RNA comprising SEQ ID NO: 20) and about 5 ug of RNA encoding a SARS-CoV-2 S protein of a BA.4/5 Omicron variant (e.g., RNA comprising SEQ ID NO: 72).

In one embodiment, the paediatric population comprises or consists of subjects less than 5 years of age including subjects 2 to less than 5 years of age, subjects 12 to less than 24 months of age, subjects 7 to less than 12 months of age, subjects 6 to less than 12 months of age and/or subjects less than 6 months of age. In this embodiment, treatments may comprise 2 vaccinations, e.g., 21 to 42 days apart, e.g., 21 days apart, wherein, in various embodiments, the vaccine is administered in an amount of 3 μg, 6 μg, 10 μg, 20 μg, or 30 μg RNA per dose, e.g., by intramuscular administration. In some such embodiments, an mRNA composition described herein is administered to subjects of age 2 to less than 5 and each dose comprises about 3 ug of mRNA. In some such embodiments, an mRNA composition described herein is administered to subjects of about 6 months to less than about 5 years and each dose comprises about 3 ug of mRNA. In some such embodiments, an mRNA composition described herein is administered to subjects of age 2 to less than 5 and each dose comprises about 6 ug of mRNA.

In some such embodiments, an mRNA composition described herein is administered to subjects of about 6 months to less than about 5 years and each dose comprises about 6 ug of mRNA. In some such embodiments, an mRNA composition described herein is administered to subjects of age 2 to less than 5 and each dose comprises about 10 ug of mRNA. In some such embodiments, an mRNA composition described herein is administered to subjects of about 6 months to less than about 5 years and each dose comprises about 10 ug of mRNA.

In some embodiments, each dose of a combination vaccine comprises about 1.5 ug of RNA encoding a SARS-CoV-2 S protein of a first variant and about 1.5 ug of RNA encoding a SARS-CoV-2 S protein of a second variant. In some embodiments, each dose comprises about 1.5 ug of RNA encoding a SARS-CoV-2 S protein of a Wuhan strain and about 1.5 ug of RNA encoding a SARS-CoV-2 S protein of an Omicron variant (e.g., a BA.1, BA.4/5, or XBB.1.5 Omicron variant). In some embodiments, each dose comprises about 1.5 ug of RNA encoding a SARS-CoV-2 S protein of a Wuhan strain (e.g., RNA comprising SEQ ID NO: 20) and about 1.5 ug of RNA encoding a SARS-CoV-2 S protein of a BA.1 Omicron variant (e.g., RNA comprising SEQ ID NO: 51). In some embodiments, each dose comprises about 1.5 ug of RNA encoding a SARS-CoV-2 S protein of a Wuhan strain (e.g., RNA comprising SEQ ID NO: 20) and about 1.5 ug of RNA encoding a SARS-CoV-2 S protein of a BA.4/5 Omicron variant (e.g., RNA comprising SEQ ID NO: 72).

In some embodiments, each dose of a combination vaccine comprises about 3 ug of RNA encoding a SARS-CoV-2 S protein of a first variant and about 3 ug of RNA encoding a SARS-CoV-2 S protein of a second variant. In some embodiments, each dose comprises about 3 ug of RNA encoding a SARS-CoV-2 S protein of a Wuhan strain and about 3 ug of RNA encoding a SARS-CoV-2 S protein of an Omicron variant (e.g., a BA.1, BA.4/5, or XBB.1.5 Omicron variant). In some embodiments, each dose comprises about 3 ug of RNA encoding a SARS-CoV-2 S protein of a Wuhan strain (e.g., RNA comprising SEQ ID NO: 20) and about 3 ug of RNA encoding a SARS-CoV-2 S protein of a BA.1 Omicron variant (e.g., RNA comprising SEQ ID NO: 51). In some embodiments, each dose comprises about 3 ug of RNA encoding a SARS-CoV-2 S protein of a Wuhan strain (e.g., RNA comprising SEQ ID NO: 20) and about 6 ug of RNA encoding a SARS-CoV-2 S protein of a BA.4/5 Omicron variant (e.g., RNA comprising SEQ ID NO: 72). In some embodiments, each dose comprises about 5 ug of RNA encoding a SARS-CoV-2 S protein of a first variant and about 5 ug of RNA encoding a SARS-CoV-2 S protein of a second variant. In some embodiments, each dose comprises about 5 ug of RNA encoding a SARS-CoV-2 S protein of a Wuhan strain and about 5 ug of RNA encoding a SARS-CoV-2 S protein of an Omicron variant (e.g., a BA.1, BA.4/5, or XBB.1.5 Omicron variant). In some embodiments, each dose comprises about 5 ug of RNA encoding a SARS-CoV-2 S protein of a Wuhan strain (e.g., RNA comprising SEQ ID NO: 20) and about 5 ug of RNA encoding a SARS-CoV-2 S protein of a BA.1 Omicron variant (e.g., RNA comprising SEQ ID NO: 51). In some embodiments, each dose comprises about 5 ug of RNA encoding a SARS-CoV-2 S protein of a Wuhan strain (e.g., RNA comprising SEQ ID NO: 20) and about 5 ug of RNA encoding a SARS-CoV-2 S protein of a BA.4/5 Omicron variant (e.g., RNA comprising SEQ ID NO: 72).

In some embodiments, an mRNA composition described herein is administered to subjects of age 12 or older and at least one dose given in a vaccination regimen (e.g., a primary vaccination regimen and/or a booster vaccination regimen) comprises about 60 ug of mRNA. In some embodiments, an mRNA composition described herein is administered to subjects of age 12 or older and at least one dose given in a vaccination regimen (e.g., a primary vaccination regimen and/or a booster vaccination regimen) comprises about 30 ug of mRNA. In some embodiments, an mRNA composition described herein is administered to subjects of age 12 or older and at least one dose given in a vaccination regimen (e.g., a primary vaccination regimen and/or a booster vaccination regimen) comprises about 90 ug of mRNA. In some embodiments, an mRNA composition described herein is administered to subjects of age 12 or older and at least one dose given in a vaccination regimen (e.g., a primary vaccination regimen and/or a booster vaccination regimen) comprises about 75 ug of mRNA. In some embodiments, an mRNA composition described herein is administered to subjects of age 12 or older and at least one dose given in a vaccination regimen (e.g., a primary vaccination regimen and/or a booster vaccination regimen) comprises about 15 ug of mRNA. In some embodiments, an mRNA composition described herein is administered to subjects of age 5 to less than 12 years of age and at least one dose given in a vaccination regimen (e.g., a primary vaccination regimen and/or a booster vaccination regimen) comprises about 10 ug of mRNA. In some embodiments, an mRNA composition described herein is administered to subjects of age 2 to less than 5 and at least one dose given in a vaccination regimen (e.g., a primary vaccination regimen and/or a booster vaccination regimen) comprises about 3 ug of mRNA.

In some embodiments, an mRNA composition described herein is administered to subjects of 6 months to less than age 2 and at least one dose given in a vaccination regimen (e.g., a primary vaccination regimen and/or a booster vaccination regimen) comprises about 3 ug or less, including, e.g., 2 ug, 1 ug, or less) of mRNA. In some embodiments, an mRNA composition described herein is administered to infants of less than 6 months and at least one dose given in a vaccination regimen (e.g., a primary vaccination regimen and/or a booster vaccination regimen) comprises about 3 ug or less (including, e.g., 2 ug, 1 ug, 0.5 ug, or less) of mRNA.

In some embodiments, an mRNA composition described herein is administered (e.g., by intramuscular injection) as a single dose. In some embodiments, a single dose comprise a single RNA encoding a SARS-CoV-2 S protein or an immunogenic fragment thereof (e.g., an RBD domain). In some embodiments, a single dose comprise at least two RNAs described herein, for example, each RNA encoding a SARS-CoV-2 S protein or an immunogenic fragment thereof (e.g., an RBD domain) from different strains. In some embodiments, such at least two RNAs described herein can be administered as a single mixture. For example, in some such embodiments, two separate RNA compositions described herein can be mixed to generate a single mixture prior to injection. In some embodiments, such at least two RNAs described herein can be administered as two separate compositions, which, for example, can be administered at different injection sites (e.g., on different arms, or different sites on the same arm).

In some embodiments, a dose administered to subjects in need thereof may comprise administration of a single mRNA composition described herein.

In some embodiments, a dose administered to subjects in need thereof may comprise administration of at least two or more (including, e.g., at least three or more) different drug products/formulations. For example, in some embodiments, at least two or more different drug products/formulations may comprise at least two different mRNA compositions described herein (e.g., in some embodiments each comprising a different RNA construct).

In some embodiments, an mRNA composition disclosed herein may be administered in conjunction with a vaccine targeting a different infectious agent. In some embodiments, the different infectious agent is one that increases the likelihood of a subject experiencing deleterious symptoms when co-infected with SARS-CoV-2 and the infectious agent. In some embodiments, the infectious agent is one that increases the infectivity of SARS-CoV-2 when a subject is co-infected with SARS-CoV-2 and the infectious agent. In some embodiments, at least one mRNA composition described herein may be administered in combination with a vaccine that targets influenza. In some embodiments, at least two or more different drug products/formulations may comprise at least one mRNA composition described herein and a vaccine targeting a different infectious agent (e.g., an influenza vaccine). In some embodiments, different drug products/formulations are separately administered. In some embodiments, such different drug product/formulations are separately administered at the same time (e.g., at the same vaccination session) at different sites of a subject (e.g., at different arms of the subject).

In one embodiment, at least two doses are administered. For example, a second dose may be administered about 21 days following administration of the first dose.

In some embodiments, compositions and methods disclosed herein can be used in accordance with an exemplary vaccination regimen as illustrated in FIG. 1.

### Exemplary Primary Dosing Regimens

In some embodiments, subjects are administered a primary dosing regimen. A primary dosing regimen can comprise one or more doses. For example, in some embodiments, a primary dosing regimen comprises a single dose (PD₁). In some embodiments a primary dosing regimen comprises a first dose (PD₁) and a second dose (PD₂). In some embodiments, a primary dosing regimen comprises a first dose, a second dose, and a third dose (PD₃). In some embodiments, a primary dosing regimen comprises a first dose, a second dose, a third dose, and one or more additional doses (PDₙ) of any one of the pharmaceutical compositions described herein.

In some embodiments, PD₁ comprises administering 1 to 100 ug of RNA. In some embodiments, PD₁ comprises administering 1 to 60 ug of RNA In some embodiments, PD₁ comprises administering 1 to 50 ug of RNA. In some embodiments, PD₁ comprises administering 1 to 30 ug of RNA. In some embodiments, PD₁ comprises administering about 3 ug of RNA. In some embodiments, PD₁ comprises administering about 5 ug of RNA. In some embodiments, PD₁ comprises administering about 10 ug of RNA. In some embodiments, PD₁ comprises administering about 15 ug of RNA. In some embodiments, PD₁ comprises administering about 20 ug of RNA. In some embodiments, PD₁ comprises administering about 30 ug of RNA. In some embodiments, PD₁ comprises administering about 50 ug of RNA. In some embodiments, PD₁ comprises administering about 60 ug of RNA.

In some embodiments, PD₂ comprises administering 1 to 100 ug of RNA. In some embodiments, PD₂ comprises administering 1 to 60 ug of RNA. In some embodiments, PD₂ comprises administering 1 to 50 ug of RNA. In some embodiments, PD₂ comprises administering 1 to 30 ug of RNA. In some embodiments, PD₂ comprises administering about 3 ug. In some embodiments, PD₂ comprises administering about 5 ug of RNA. In some embodiments, PD₂ comprises administering about 10 ug of RNA. In some embodiments, PD₂ comprises administering about 15 ug of RNA. In some embodiments, PD₂ comprises administering about 20 ug RNA. In some embodiments, PD₂ comprises administering about 30 ug of RNA. In some embodiments, PD₂ comprises administering about 50 ug of RNA. In some embodiments, PD₂ comprises administering about 60 ug of RNA.

In some embodiments, PD₃ comprises administering 1 to 100 ug of RNA. In some embodiments, PD₃ comprises administering 1 to 60 ug of RNA. In some embodiments, PD₃ comprises administering 1 to 50 ug of RNA. In some embodiments, PD₃ comprises administering 1 to 30 ug of RNA. In some embodiments, PD₃ comprises administering about 3 ug of RNA. In some embodiments, PD₃ comprises administering about 5 ug of RNA. In some embodiments, PD₃ comprises administering about 10 ug of RNA. In some embodiments, PD₃ comprises administering about 15 ug of RNA. In some embodiments, PD₃ comprises administering about 20 ug of RNA. In some embodiments, PD₃ comprises administering about 30 ug of RNA. In some embodiments, PD₃ comprises administering about 50 ug of RNA. In some embodiments, PD₃ comprises administering about 60 ug of RNA.

In some embodiments, PDₙ comprises administering 1 to 100 ug of RNA. In some embodiments, PDₙ comprises administering 1 to 60 ug of RNA. In some embodiments, PDₙ comprises administering 1 to 50 ug of RNA. In some embodiments, PDₙ comprises administering 1 to 30 ug of RNA. In some embodiments, PDₙ comprises administering about 3 ug of RNA. In some embodiments, PDₙ comprises administering about 5 ug of RNA. In some embodiments, PDₙ comprises administering about 10 ug of RNA. In some embodiments, PDₙ comprises administering about 15 ug of RNA. In some embodiments, PDₙ comprises administering about 20 ug of RNA. In some embodiments, PDₙ comprises administering about 30 ug of RNA. In some embodiments, PDₙ comprises administering about 50 ug of RNA. In some embodiments, PDₙ comprises administering about 60 ug of RNA.

In some embodiments, PD₁ comprises an RNA encoding a SARS-CoV-2 Spike protein or an immunogenic fragment thereof from the Wuhan strain. In some embodiments, PD₁ comprises an RNA encoding a Spike protein or an immunogenic fragment thereof from a SARS-CoV-2 strain that is prevalent and/or spreading rapidly in a relevant jurisdiction. In some embodiments, PD₁ comprises an RNA encoding a SARS-CoV-2 Spike protein or an immunogenic fragment thereof comprising one or more mutations from an alpha variant. In some embodiments, PD₁ comprises an RNA encoding a SARS-CoV-2 Spike protein or an immunogenic fragment thereof comprising one or more mutations from a delta variant. In some embodiments, PD₁ comprises an RNA encoding a SARS-CoV-2 Spike protein or an immunogenic fragment thereof comprising one or more mutations from a beta variant. In some embodiments, PD₁ comprises an RNA encoding a SARS-CoV-2 Spike protein or an immunogenic fragment thereof comprising one or more mutations from an Omicron variant. In some embodiments, PD₁ comprises an RNA encoding a SARS-CoV-2 Spike protein or an immunogenic fragment thereof from the Wuhan strain and one or more additional RNAs encoding a Spike protein or an immunogenic fragment thereof from a SARS-CoV-2 strain that is prevalent and/or spreading rapidly in a relevant jurisdiction. In some embodiments, PD₁ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an alpha variant. In some embodiments, PD₁ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, PD₁ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, PD₁ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, PD₂ comprises an RNA encoding a SARS-CoV-2 Spike protein or an immunogenic fragment thereof from the Wuhan strain. In some embodiments, PD₂ comprises an RNA encoding a Spike protein or an immunogenic fragment thereof from a SARS-CoV-2 strain that is prevalent and/or spreading rapidly in a relevant jurisdiction. In some embodiments, PD₂ comprises an RNA encoding a SARS-CoV-2 Spike protein or an immunogenic fragment thereof comprising one or more mutations from an alpha variant. In some embodiments, PD₂ comprises an RNA encoding a SARS-CoV-2 Spike protein or an immunogenic fragment thereof comprising one or more mutations from a delta variant. In some embodiments, PD₂ comprises an RNA encoding a SARS-CoV-2 Spike protein or an immunogenic fragment thereof comprising one or more mutations from a beta variant. In some embodiments, PD₂ comprises an RNA encoding a SARS-CoV-2 Spike protein or an immunogenic fragment thereof comprising one or more mutations from an Omicron variant. In some embodiments, PD₂ comprises an RNA encoding a SARS-CoV-2 Spike protein or an immunogenic fragment thereof from the Wuhan strain and one or more additional RNAs encoding a Spike protein or an immunogenic fragment thereof from a SARS-CoV-2 strain that is prevalent and/or spreading rapidly in a relevant jurisdiction. In some embodiments, PD₂ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an alpha variant.

In some embodiments, PD₂ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, PD₂ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, PD₂ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, PD₃ comprises an RNA encoding a SARS-CoV-2 Spike protein or an immunogenic fragment thereof from the Wuhan strain. In some embodiments, PD₃ comprises an RNA encoding a Spike protein or an immunogenic fragment thereof from a SARS-CoV-2 strain that is prevalent and/or spreading rapidly in a relevant jurisdiction. In some embodiments, PD₃ comprises an RNA encoding a SARS-CoV-2 Spike protein or an immunogenic fragment thereof comprising one or more mutations from an alpha variant. In some embodiments, PD₃ comprises an RNA encoding a SARS-CoV-2 Spike protein or an immunogenic fragment thereof comprising one or more mutations from a delta variant. In some embodiments, PD₃ comprises an RNA encoding a SARS-CoV-2 Spike protein or an immunogenic fragment thereof comprising one or more mutations from a beta variant. In some embodiments, PD₃ comprises an RNA encoding a SARS-CoV-2 Spike protein or an immunogenic fragment thereof comprising one or more mutations from an Omicron variant. In some embodiments, PD₃ comprises an RNA encoding a SARS-CoV-2 Spike protein or an immunogenic fragment thereof from the Wuhan strain and one or more additional RNAs encoding a Spike protein or an immunogenic fragment thereof from a SARS-CoV-2 strain that is prevalent and/or spreading rapidly in a relevant jurisdiction. In some embodiments, PD₃ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an alpha variant.

In some embodiments, PD₃ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, PD₃ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, PD₃ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, PDₙ comprises an RNA encoding a SARS-CoV-2 Spike protein or an immunogenic fragment thereof from the Wuhan strain. In some embodiments, PDₙ comprises an RNA encoding a Spike protein or an immunogenic fragment thereof from a SARS-CoV-2 strain that is prevalent and/or spreading rapidly in a relevant jurisdiction. In some embodiments, PDₙ comprises an RNA encoding a SARS-CoV-2 Spike protein or an immunogenic fragment thereof comprising one or more mutations from an alpha variant. In some embodiments, PDₙ comprises an RNA encoding a SARS-CoV-2 Spike protein or an immunogenic fragment thereof comprising one or more mutations from a delta variant. In some embodiments, PDₙ comprises an RNA encoding a SARS-CoV-2 Spike protein or an immunogenic fragment thereof comprising one or more mutations from a beta variant. In some embodiments, PDₙ comprises an RNA encoding a SARS-CoV-2 Spike protein or an immunogenic fragment thereof comprising one or more mutations from an Omicron variant. In some embodiments, PDₙ comprises an RNA encoding a SARS-CoV-2 Spike protein or an immunogenic fragment thereof from the Wuhan strain and one or more additional RNAs encoding a Spike protein or an immunogenic fragment thereof from a SARS-CoV-2 strain that is prevalent and/or spreading rapidly in a relevant jurisdiction. In some embodiments, PDₙ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an alpha variant. In some embodiments, PDₙ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, PDₙ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, PDₙ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, PD₁, PD₂, PD₃, and PDₙ can each independently comprise a plurality of (e.g., at least two) mRNA compositions described herein. In some embodiments PD₁, PD₂, PD₃, and PDₙ can each independently comprise a first and a second mRNA composition. In some embodiments, at least one of a plurality of mRNA compositions comprises BNT162b2 (e.g., as described herein). In some embodiments, at least one of a plurality of mRNA compositions comprises an mRNA encoding a SARS-CoV-2 S protein or an immunogenic fragment thereof from a different SARS-CoV-2 variant. In some embodiments, at least one of a plurality of mRNA compositions comprises an mRNA encoding a SARS-CoV-2 S protein or an immunogenic fragment thereof from a Wuhan strain of SARS-CoV-2. In some embodiments, at least one of a plurality of mRNA compositions comprises an RNA encoding a SARS-CoV-2 S protein or an immunogenic fragment thereof comprising one or more mutations from a variant that is prevalent and/or spreading rapidly in a relevant jurisdiction. In some embodiments, at least one of a plurality of mRNA compositions comprises an mRNA encoding a SARS-CoV-2 S protein or an immunogenic fragment thereof comprising one or more mutations from an alpha variant. In some embodiments, at least one of a plurality of mRNA compositions comprises an mRNA encoding a SARS-CoV-2 S protein or an immunogenic fragment thereof comprising one or more mutations from a delta variant. In some embodiments, at least one of a plurality of mRNA compositions comprises an mRNA encoding a SARS-CoV-2 S protein or an immunogenic fragment thereof comprising one or more mutations from an Omicron variant.

In some embodiments, a plurality of mRNA compositions given in PD₁, PD₂, PD₃, and/or PDₙ can each independently comprise at least two different mRNA constructs (e.g., differing in at protein-encoding sequences). For example, in some embodiments a plurality of mRNA compositions given in PD₁, PD₂, PD₃, and/or PDₙ can each independently comprise an mRNA encoding a SARS-CoV-2 S protein or an immunogenic fragment thereof from a Wuhan strain of SARS-CoV-2 and an mRNA encoding a SARS-CoV-2 S protein or an immunogenic fragment thereof comprising one or more mutations from a variant that is prevalent and/or spreading rapidly in a relevant jurisdiction. In some embodiments a plurality of mRNA compositions given in PD₁, PD₂, PD₃, and/or PDₙ can each independently comprise an mRNA encoding a SARS-CoV-2 S protein or an immunogenic fragment thereof derived from a Wuhan strain of SARS-CoV-2 and an mRNA encoding a SARS-CoV-2 S protein or an immunogenic fragment thereof comprising one or more mutations from a variant that is prevalent and/or spreading rapidly in a relevant jurisdiction. In some such embodiments, a variant can be an alpha variant. In some such embodiments, a variant can be a delta variant. In some such embodiments a variant can be an Omicron variant.

In some embodiments, each of a plurality of mRNA compositions given in PD₁, PD₂, PD₃, and/or PDₙ can independently comprise at least two mRNAs, each encoding a SARS-CoV-2 S protein or an immunogenic fragment thereof comprising one or more mutations from a distinct variant that is prevalent and/or spreading rapidly in a relevant jurisdiction. In some embodiments, each of a plurality of mRNA compositions given in PD₁, PD₂, PD₃, and/or PDₙ can independently comprise an mRNA encoding a SARS-CoV-2 S protein or an immunogenic fragment thereof from an alpha variant and an mRNA encoding a SARS-CoV-2 S protein or an immunogenic fragment thereof comprising one or more mutations from a delta variant. In some embodiments, each of a plurality of mRNA compositions given in PD₁, PD₂, PD₃, and/or PDₙ can independently comprise an mRNA encoding a SARS-CoV-2 S protein or an immunogenic fragment thereof from an alpha variant and an mRNA encoding a SARS-CoV-2 S protein or an immunogenic fragment thereof comprising one or more mutations from an Omicron variant. In some embodiments, each of a plurality of mRNA compositions given in PD₁, PD₂, PD₃, and/or PDₙ can independently comprise an mRNA encoding a SARS-CoV-2 S protein or an immunogenic fragment thereof from a delta variant and an mRNA encoding a SARS-CoV-2 S protein or an immunogenic fragment thereof comprising one or more mutations from an Omicron variant.

In some embodiments, PD₁, PD₂, PD₃, and/or PDₙ each comprise a plurality of mRNA compositions, wherein each mRNA composition is separately administered to a subject. For example, in some embodiments each mRNA composition is administered via intramuscular injection at different injection sites. For example, in some embodiments, a first and second mRNA composition given in PD₁, PD₂, PD₃, and/or PDₙ are separately administered to different arms of a subject via intramuscular injection.

In some embodiments, PD₁, PD₂, PD₃, and/or PDₙ comprise administering a plurality of RNA molecules, wherein each RNA molecule encodes a Spike protein comprising mutations from a different SARS-CoV-2 variant, and wherein the plurality of RNA molecules are administered to the subject in a single formulation. In some embodiments, the single formulation comprises an RNA encoding a Spike protein or an immunogenic variant thereof from the Wuhan strain and an RNA encoding a SARS-CoV-2 Spike protein or an immunogenic fragment thereof comprising one or more mutations from an alpha variant. In some embodiments, the single formulation comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, the single formulation comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, the single formulation comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, the length of time between PD₁ and PD₂ (PI₁) is at least about 1 week, at least about 2 weeks, at least about 3 weeks, or at least about 4 weeks. In some embodiments, PI₁ is about 1 week to about 12 weeks. In some embodiments, PI₁ is about 1 week to about 10 weeks. In some embodiments, PI₁ is about 2 weeks to about 10 weeks. In some embodiments, PI₁ is about 2 weeks to about 8 weeks. In some embodiments, PI₁ is about 3 weeks to about 8 weeks. In some embodiments, PI₁ is about 4 weeks to about 8 weeks. In some embodiments, PI₁ is about 6 weeks to about 8 weeks. In some embodiments PI₁ is about 3 to about 4 weeks. In some embodiments, PI₁ is about 1 week. In some embodiments, PI₁ is about 2 weeks. In some embodiments, PI₁ is about 3 weeks. In some embodiments, PI₁ is about 4 weeks. In some embodiments, PI₁ is about 5 weeks. In some embodiments, PI₁ is about 6 weeks. In some embodiments, PI₁ is about 7 weeks. In some embodiments, PI₁ is about 8 weeks. In some embodiments, PI₁ is about 9 weeks. In some embodiments, PI₁ is about 10 weeks. In some embodiments, PI₁ is about 11 weeks. In some embodiments, PI₁ is about 12 weeks.

In some embodiments, the length of time between PD₂ and PD₃ (PI₂) is at least about 1 week, at least about 2 weeks, or at least about 3 weeks. In some embodiments, PI₂ is about 1 week to about 12 weeks. In some embodiments, PI₂ is about 1 week to about 10 weeks. In some embodiments, PI₂ is about 2 weeks to about 10 weeks. In some embodiments, PI₂ is about 2 weeks to about 8 weeks. In some embodiments, PI₂ is about 3 weeks to about 8 weeks. In some embodiments, PI₂ is about 4 weeks to about 8 weeks. In some embodiments, PI₂ is about 6 weeks to about 8 weeks. In some embodiments PI₂ is about 3 to about 4 weeks. In some embodiments, PI₂ is about 1 week. In some embodiments, PI₂ is about 2 weeks. In some embodiments, PI₂ is about 3 weeks. In some embodiments, PI₂ is about 4 weeks. In some embodiments, PI₂ is about 5 weeks. In some embodiments, PI₂ is about 6 weeks. In some embodiments, PI₂ is about 7 weeks. In some embodiments, PI₂ is about 8 weeks. In some embodiments, PI₂ is about 9 weeks. In some embodiments, PI₂ is about 10 weeks. In some embodiments, PI₂ is about 11 weeks. In some embodiments, PI₂ is about 12 weeks.

In some embodiments, the length of time between PD₃ and a subsequent dose that is part of the Primary Dosing Regimen, or between doses for any dose beyond PD₃ (PIₙ) is each separately and independently selected from: about 1 week or more, about 2 weeks or more, or about 3 weeks or more. In some embodiments, PIₙ is about 1 week to about 12 weeks. In some embodiments, PIₙ is about 1 week to about 10 weeks. In some embodiments, PIₙ is about 2 weeks to about 10 weeks. In some embodiments, PIₙ is about 2 weeks to about 8 weeks. In some embodiments, PIₙ is about 3 weeks to about 8 weeks. In some embodiments, PIₙ is about 4 weeks to about 8 weeks. In some embodiments, PIₙ is about 6 weeks to about 8 weeks. In some embodiments PIₙ is about 3 to about 4 weeks. In some embodiments, PI₂ is about 1 week. In some embodiments, PIₙ is about 2 weeks. In some embodiments, PIₙ is about 3 weeks. In some embodiments, PIₙ is about 4 weeks. In some embodiments, PIₙ is about 5 weeks. In some embodiments, PIₙ is about 6 weeks. In some embodiments, PIₙ is about 7 weeks. In some embodiments, PIₙ is about 8 weeks. In some embodiments, PIₙ is about 9 weeks. In some embodiments, PIₙ is about 10 weeks. In some embodiments, PIₙ is about 11 weeks. In some embodiments, PIₙ is about 12 weeks.

In some embodiments, one or more compositions adminstered in PD₁ are formulated in a Tris buffer. In some embodiments, one or more compositions administered in PD₂ are formulated in a Tris buffer. In some embodiments, one or more compositions administering in PD₃ are formulated in a Tris buffer. In some embodiments, one or more compositions administered in PDₙ are formulated in a Tris buffer.

In some embodiments, the primary dosing regimen comprises administering two or more mRNA compositions described herein, and at least two of the mRNA compositions have different formulations. In some embodiments, the primary dosing regimen comprises PD₁ and PD₂, where PD₁ comprises administering an mRNA formulated in a Tris buffer and PD₂ comprises administering an mRNA formulated in a PBS buffer. In some embodiments, the primary dosing regimen comprises PD₁ and PD₂, where PD₁ comprises administering an mRNA formulated in a PBS buffer and PD₂ comprises administering an mRNA formulated in a Tris buffer.

In some embodiments, one or more mRNA compositions given in PD₁, PD₂, PD₃, and/or PDₙ can be administered in combination with another vaccine. In some embodiments, another vaccine is for a disease that is not COVID-19. In some embodiments, the disease is one that increases deleterious effects of SARS-CoV-2 when a subject is coinfected with the disease and SARS-CoV-2. In some embodiments, the disease is one that increases the transmission rate of SARS-CoV-2 when a subject is coinfected with the disease and SARS-CoV-2. In some embodiments, another vaccine is a different commerically available vaccine. In some embodiments, the different commercially available vaccine is an RNA vaccine. In some embodiments, the different commercially available vaccine is a polypeptide-based vaccine. In some embodiments, another vaccine (e.g., as described herein) and one or more mRNA compositions given in PD₁, PD₂, PD₃, and/or PDₙ are separately administered, for example, in some embodiments via intramuscular injection, at different injection sites. For example, in some embodiments, an influenza vaccine and one or more SARS-CoV-2 mRNA compositions described herein given in PD₁, PD₂, PD₃, and/or PDₙ are separately administered to different arms of a subject via intramuscular injection.

### Exemplary Booster Dosing Regimens

In some embodiments, methods of vaccination disclosed herein comprise one or more Booster Dosing Regimens. The Booster Dosing Regimens disclosed herein comprise one or more doses. In some embodiments, a Booster Dosing Regimen is administered to patients who have been administered a Primary Dosing Regimen (e.g., as described herein). In some embodiments a Booster Dosing Regimen is administered to patients who have not received a pharmaceutical composition disclosed herein. In some embodiments a Booster Dosing Regimen is administered to patients who have been previously vaccinated with a COVID-19 vaccine that is different from the vaccine administered in a Primary Dosing Regimen.

In some embodiments, the length of time between the Primary Dosing Regimen and the Booster Dosing Regimen is at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 2 months, at least 3 months, at least 4 months, at least 5 months, at least 6 months, at least 7 months, at least 8 months, at least 9 months, at least 10 months, at least 11 months, or at least 12 months or longer. In some embodiments, the length of time between the Primary Dosing Regimen and the Booster Dosing Regimen is about 1 month. In some embodiments, the length of time between the Primary Dosing Regimen and the Booster Dosing Regimen is at least about 2 months. In some embodiments, the length of time between the Primary Dosing Regimen and the Booster Dosing Regimen is at least about 3 months. In some embodiments, the length of time between the Primary Dosing Regimen and the Booster Dosing Regimen is at least about 4 months. In some embodiments, the length of time between the Primary Dosing Regimen and the Booster Dosing Regimen is at least about 5 months. In some embodiments, the length of time between the Primary Dosing Regimen and the Booster Dosing Regimen is at least about 6 months. In some embodiments, the length of time between the Primary Dosing Regimen and the Booster Dosing Regimen is from about 1 month to about 48 months. In some embodiments, the length of time between the Primary Dosing Regimen and the Booster Dosing Regimen is from about 1 month to about 36 months. In some embodiments, the length of time between the primary dosing regimen and the Booster Dosing Regimen is from about 1 month to about 24 months. In some embodiments, the length of time between the Primary Dosing Regimen and the Booster Dosing Regimen is from about 2 months to about 24 months. In some embodiments, the length of time between the Primary Dosing Regimen and the Booster Dosing Regimen is from about 3 months to about 24 months. In some embodiments, the length of time between the primary dosing regimen and the Booster Dosing Regimen is from about 3 months to about 18 months. In some embodiments, the length of time between the primary dosing regimen and the Booster Dosing Regimen is from about 3 months to about 12 months. In some embodiments, the length of time between the primary dosing regimen and the Booster Dosing Regimen is from about 6 months to about 12 months. In some embodiments, the length of time between the Primary Dosing Regimen and the Booster Dosing Regimen is from about 3 months to about 9 months. In some embodiments, the length of time between the Primary Dosing Regimen and the Booster Dosing Regimen is from about 5 months to about 7 months. In some embodiments, the length of time between the Primary Dosing Regimen and the Booster Dosing Regimen is about 6 months.

In some embodiments, subjects are administered a Booster Dosing Regimen. A Booster dosing regimen can comprise one or more doses. For example, in some embodiments, a Booster Dosing Regimen comprises a single dose (BD₁). In some embodiments a Booster Dosing Regimen comprises a first dose (BD₁) and a second dose (BD₂). In some embodiments, a Booster Dosing Regimen comprises a first dose, a second dose, and a third dose (BD₃). In some embodiments, a Booster Dosing Regimen comprises a first dose, a second dose, a third dose, and one or more additional doses (BDₙ) of any one of the pharmaceutical compositions described herein.

In some embodiments, BD₁ comprises administering 1 to 100 ug of RNA. In some embodiments, BD₁ comprises administering 1 to 60 ug of RNA. In some embodiments, BD₁ comprises administering 1 to 50 ug of RNA. In some embodiments, BD₁ comprises administering 1 to 30 ug of RNA. In some embodiments, BD₁ comprises administering about 3 ug of RNA. In some embodiments, BD₁ comprises administering about 5 ug of RNA. In some embodiments, BD₁ comprises administering about 10 ug of RNA. In some embodiments, BD₁ comprises administering about 15 ug of RNA. In some embodiments, BD₁ comprises administering about 20 ug of RNA. In some embodiments, BD₁ comprises administering about 30 ug of RNA. In some embodiments, BD₁ comprises administering about 50 ug of RNA. In some embodiments, BD₁ comprises administering about 60 ug of RNA.

In some embodiments, BD₂ comprises administering 1 to 100 ug of RNA. In some embodiments, BD₂ comprises administering 1 to 60 ug of RNA. In some embodiments, BD₂ comprises administering 1 to 50 ug of RNA. In some embodiments, BD₂ comprises administering 1 to 30 ug of RNA. In some embodiments, BD₂ comprises administering about 3 ug. In some embodiments, BD₂ comprises administering about 5 ug of RNA. In some embodiments, BD₂ comprises administering about 10 ug of RNA. In some embodiments, BD₂ comprises administering about 15 ug of RNA. In some embodiments, BD₂ comprises administering about 20 ug RNA. In some embodiments, BD₂ comprises administering about 30 ug of RNA. In some embodiments, BD₂ comprises administering about 50 ug of RNA. In some embodiments, BD₂ comprises administering about 60 ug of RNA.

In some embodiments, BD₃ comprises administering 1 to 100 ug of RNA. In some embodiments, BD₃ comprises administering 1 to 60 ug of RNA. In some embodiments, BD₃ comprises administering 1 to 50 ug of RNA. In some embodiments, BD₃ comprises administering 1 to 30 ug of RNA. In some embodiments, BD₃ comprises administering about 3 ug of RNA. In some embodiments, BD₃ comprises administering about 5 ug of RNA. In some embodiments, BD₃ comprises administering about 10 ug of RNA. In some embodiments, BD₃ comprises administering about 15 ug of RNA. In some embodiments, BD₃ comprises administering about 20 ug of RNA. In some embodiments, BD₃ comprises administering about 30 ug of RNA. In some embodiments, BD₃ comprises administering about 50 ug of RNA. In some embodiments, BD₃ comprises administering about 60 ug of RNA.

In some embodiments, BDₙ comprises administering 1 to 100 ug of RNA. In some embodiments, BDₙ comprises administering 1 to 60 ug of RNA. In some embodiments, BDₙ comprises administering 1 to 50 ug of RNA. In some embodiments, BDₙ comprises administering 1 to 30 ug of RNA. In some embodiments, BDₙ comprises administering about 3 ug of RNA. In some embodiments, BDₙ comprises administering about 5 ug of RNA. In some embodiments, BDₙ comprises administering about 10 ug of RNA. In some embodiments, BDₙ comprises administering about 15 ug of RNA. In some embodiments, BDₙ comprises administering about 20 ug of RNA. In some embodiments, BDₙ comprises administering about 30 ug of RNA. In some embodiments, BDₙ comprises administering about 60 ug of RNA. In some embodiments, BDₙ comprises administering about 50 ug of RNA.

In some embodiments, BD₁ comprises an RNA encoding a SARS-CoV-2 Spike protein or an immunogenic fragment thereof from the Wuhan strain. In some embodiments, BD₁ comprises an RNA encoding a Spike protein or an immunogenic fragment thereof from a SARS-CoV-2 strain that is prevalent and/or spreading rapidly in a relevant jurisdiction. In some embodiments, BD₁ comprises an RNA encoding a SARS-CoV-2 Spike protein or an immunogenic fragment thereof comprising one or more mutations from an alpha variant. In some embodiments, BD₁ comprises an RNA encoding a SARS-CoV-2 Spike protein or an immunogenic fragment thereof comprising one or more mutations from a delta variant. In some embodiments, BD₁ comprises an RNA encoding a SARS-CoV-2 Spike protein or an immunogenic fragment thereof comprising one or more mutations from a beta variant. In some embodiments, BD₁ comprises an RNA encoding a SARS-CoV-2 Spike protein or an immunogenic fragment thereof comprising one or more mutations from an Omicron variant.

In some embodiments, BD₁ comprises an RNA encoding a SARS-CoV-2 Spike protein or an immunogenic fragment thereof from the Wuhan strain and one or more RNA encoding a Spike protein or an immunogenic fragment thereof from a SARS-CoV-2 strain that is prevalent and/or spreading rapidly in a relevant jurisdiction. In some embodiments, BD₁ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a alpha variant. In some embodiments, BD₁ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, BD₁ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, BD₁ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, BD₂ comprises an RNA encoding a SARS-CoV-2 Spike protein or an immunogenic fragment thereof from the Wuhan strain. In some embodiments, BD₂ comprises an RNA encoding a Spike protein or an immunogenic fragment thereof from a SARS-CoV-2 strain that is prevalent and/or spreading rapidly in a relevant jurisdiction. In some embodiments, BD₂ comprises an RNA encoding a SARS-CoV-2 Spike protein or an immunogenic fragment thereof comprising one or more mutations from an alpha variant. In some embodiments, BD₂ comprises an RNA encoding a SARS-CoV-2 Spike protein or an immunogenic fragment thereof comprising one or more mutations from a delta variant. In some embodiments, BD₂ comprises an RNA encoding a SARS-CoV-2 Spike protein or an immunogenic fragment thereof comprising one or more mutations from a beta variant. In some embodiments, BD₂ comprises an RNA encoding a SARS-CoV-2 Spike protein or an immunogenic fragment thereof comprising one or more mutations from an Omicron variant. In some embodiments, BD₂ comprises an RNA encoding a SARS-CoV-2 Spike protein or an immunogenic fragment thereof from the Wuhan strain and one or more RNA encoding a Spike protein or an immunogenic fragment thereof from a SARS-CoV-2 strain that is prevalent and/or spreading rapidly in a relevant jurisdiction. In some embodiments, BD₂ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a alpha variant.

In some embodiments, BD₂ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, BD₂ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, BD₂ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, BD₃ comprises an RNA encoding a SARS-CoV-2 Spike protein or an immunogenic fragment thereof from the Wuhan strain. In some embodiments, BD₃ comprises an RNA encoding a Spike protein or an immunogenic fragment thereof from a SARS-CoV-2 strain that is prevalent and/or spreading rapidly in a relevant jurisdiction. In some embodiments, BD₃ comprises an RNA encoding a SARS-CoV-2 Spike protein or an immunogenic fragment thereof comprising one or more mutations from an alpha variant. In some embodiments, BD₃ comprises an RNA encoding a SARS-CoV-2 Spike protein or an immunogenic fragment thereof comprising one or more mutations from a delta variant. In some embodiments, BD₃ comprises an RNA encoding a SARS-CoV-2 Spike protein or an immunogenic fragment thereof comprising one or more mutations from a beta variant. In some embodiments, BD₃ comprises an RNA encoding a SARS-CoV-2 Spike protein or an immunogenic fragment thereof comprising one or more mutations from an Omicron variant.

In some embodiments, BD₃ comprises an RNA encoding a SARS-CoV-2 Spike protein or an immunogenic fragment thereof from the Wuhan strain and one or more RNA encoding a Spike protein or an immunogenic fragment thereof from a SARS-CoV-2 strain that is prevalent and/or spreading rapidly in a relevant jurisdiction. In some embodiments, BD₃ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a alpha variant. In some embodiments, BD₃ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, BD₃ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, BD₃ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, BDₙ comprises an RNA encoding a SARS-CoV-2 Spike protein or an immunogenic fragment thereof from the Wuhan strain. In some embodiments, BDₙ comprises an RNA encoding a Spike protein or an immunogenic fragment thereof from a SARS-CoV-2 strain that is prevalent and/or spreading rapidly in a relevant jurisdiction. In some embodiments, BDₙ comprises an RNA encoding a SARS-CoV-2 Spike protein or an immunogenic fragment thereof comprising one or more mutations from an alpha variant. In some embodiments, BDₙ comprises an RNA encoding a SARS-CoV-2 Spike protein or an immunogenic fragment thereof comprising one or more mutations from a delta variant. In some embodiments, BDₙ comprises an RNA encoding a SARS-CoV-2 Spike protein or an immunogenic fragment thereof comprising one or more mutations from a beta variant. In some embodiments, BDₙ comprises an RNA encoding a SARS-CoV-2 Spike protein or an immunogenic fragment thereof comprising one or more mutations from an Omicron variant.

In some embodiments, BDₙ comprises an RNA encoding a SARS-CoV-2 Spike protein or an immunogenic fragment thereof from the Wuhan strain and one or more RNA encoding a Spike protein or an immunogenic fragment thereof from a SARS-CoV-2 strain that is prevalent and/or spreading rapidly in a relevant jurisdiction. In some embodiments, BDₙ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a alpha variant. In some embodiments, BDₙ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a delta variant. In some embodiments, BDₙ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from a beta variant. In some embodiments, BDₙ comprises an RNA encoding a SARS-CoV-2 Spike protein or an immunogenic fragment thereof from the Wuhan strain and an RNA encoding a SARS-CoV-2 Spike protein or an immunogenic fragment thereof comprising one or more mutations from an Omicron variant.

In some embodiments, BD₁, BD₂, BD₃, and BDₙ can each independently comprise a plurality of (e.g., at least two) mRNA compositions described herein. In some embodiments BD₁, BD₂, BD₃, and BDₙ can each independently comprise a first and a second mRNA composition. In some embodiments, BD₁, BD₂, BD₃, and BDₙ can each independently comprise a plurality of (e.g., at least two) mRNA compositions, wherein, at least one of the plurality of mRNA compositions comprises BNT162b2 (e.g., as described herein). In some embodiments, at least one of a plurality of mRNA compositions comprises an mRNA encoding a SARS-CoV-2 S protein or an immunogenic fragment thereof from a different SARS-CoV-2 variant (e.g., a variant that is prevalent or rapidly spreading in a relevant jurisdiction, e.g., a variant disclosed herein). In some embodiments, at least one of a plurality of mRNA compositions comprises an mRNA encoding a SARS-CoV-2 S protein or an immunogenic fragment thereof from a Wuhan strain of SARS-CoV-2. In some embodiments, at least one of a plurality of mRNA compositions comprises an RNA encoding a SARS-CoV-2 S protein or an immunogenic fragment thereof comprising one or more mutations from a variant that is prevalent and/or spreading rapidly in a relevant jurisdiction. In some embodiments, at least one of a plurality of mRNA compositions comprises an mRNA encoding a SARS-CoV-2 S protein or an immunogenic fragment thereof comprising one or more mutations from an alpha variant. In some embodiments, at least one of a plurality of mRNA compositions comprises an mRNA encoding a SARS-CoV-2 S protein or an immunogenic fragment thereof comprising one or more mutations from a delta variant. In some embodiments, at least one of a plurality of mRNA compositions comprises an mRNA encoding a SARS-CoV-2 S protein or an immunogenic fragment thereof comprising one or more mutations from an Omicron variant.

In some embodiments, a plurality of mRNA compositions given in BD₁, BD₂, BD₃, and/or BDₙ can each independently comprise at least two different mRNA constructs (e.g., mRNA constructs having differing protein-encoding sequences). For example, in some embodiments a plurality of mRNA compositions given in BD₁, BD₂, BD₃, and/or BDₙ can each independently comprise an mRNA encoding a SARS-CoV-2 S protein or an immunogenic fragment thereof from a Wuhan strain of SARS-CoV-2 and an mRNA encoding a SARS-CoV-2 S protein or an immunogenic fragment thereof comprising one or more mutations from a variant that is prevalent and/or spreading rapidly in a relevant jurisdiction. In some embodiments a plurality of mRNA compositions given in BD₁, BD₂, BD₃, and/or BDₙ can each independently comprise an mRNA encoding a SARS-CoV-2 S protein or an immunogenic fragment thereof derived from a Wuhan strain of SARS-CoV-2 and an mRNA encoding a SARS-CoV-2 S protein or an immunogenic fragment thereof comprising one or more mutations from a variant that is prevalent and/or spreading rapidly in a relevant jurisdiction. In some such embodiments, a variant can be an alpha variant. In some such embodiments, a variant can be a delta variant. In some such embodiments a variant can be an Omicron variant (e.g., a BA.4/5, BA.1, BA.2, XBB, XBB.1, XBB.1.5, XBB.1.16, XBB.2.3, XBB.2.3.2, or BQ.1 Omicron variant).

In some embodiments, a plurality of mRNA compositions given in BD₁, BD₂, BD₃, and/or BDₙ can each independently comprise at least two mRNAs each encoding a SARS-CoV-2 S protein or an immunogenic fragment thereof comprising one or more mutations from a distinct variant that is prevalent and/or spreading rapidly in a relevant jurisdiction. In some embodiments a plurality of mRNA compositions given in BD₁, BD₂, BD₃, and/or BDₙ can each independently comprise an mRNA encoding a SARS-CoV-2 S protein or an immunogenic fragment thereof from an alpha variant and an mRNA encoding a SARS-CoV-2 S protein or an immunogenic fragment thereof comprising one or more mutations from a delta variant. In some embodiments a plurality of mRNA compositions given in BD₁, BD₂, BD₃, and/or BDₙ can each independently comprise an mRNA encoding a SARS-CoV-2 S protein or an immunogenic fragment thereof from an alpha variant and an mRNA encoding a SARS-CoV-2 S protein or an immunogenic fragment thereof comprising one or more mutations from an Omicron variant (e.g., a BA.4/5, BA.1, BA.2, XBB, XBB.1, XBB.1.5, XBB.1.16, XBB.2.3, XBB.2.3.2, or BQ.1 Omicron variant). In some embodiments a plurality of mRNA compositions given in BD₁, BD₂, BD₃, and/or BDₙ can each independently comprise an mRNA encoding a SARS-CoV-2 S protein or an immunogenic fragment thereof derived from a Wuhan strain of SARS-CoV-2 and an mRNA encoding a SARS-CoV-2 S protein or an immunogenic fragment thereof comprising one or more mutations from a variant that is prevalent and/or spreading rapidly in a relevant jurisdiction. In some such embodiments, a variant can be an alpha variant. In some such embodiments, a variant can be a delta variant. In some such embodiments a variant can be an Omicron variant.

In some embodiments, a plurality of mRNA compositions given in BD₁, BD₂, BD₃, and/or BDₙ can each independently comprise at least two mRNAs each encoding a SARS-CoV-2 S protein or an immunogenic fragment thereof comprising one or more mutations from a distinct variant that is prevalent and/or spreading rapidly in a relevant jurisdiction. In some embodiments a plurality of mRNA compositions given in BD₁, BD₂, BD₃, and/or BDₙ can each independently comprise an mRNA encoding a SARS-CoV-2 S protein or an immunogenic fragment thereof from an alpha variant and an mRNA encoding a SARS-CoV-2 S protein or an immunogenic fragment thereof comprising one or more mutations from a delta variant. In some embodiments a plurality of mRNA compositions given in BD₁, BD₂, BD₃, and/or BDₙ can each independently comprise an mRNA encoding a SARS-CoV-2 S protein or an immunogenic fragment thereof from an alpha variant and an mRNA encoding a SARS-CoV-2 S protein or an immunogenic fragment thereof comprising one or more mutations from an Omicron variant. In some embodiments a plurality of mRNA compositions given in BD₁, BD₂, BD₃, and/or BDₙ can each independently comprise an mRNA encoding a SARS-CoV-2 S protein or an immunogenic fragment thereof from a delta variant and an mRNA encoding a SARS-CoV-2 S protein or an immunogenic fragment thereof comprising one or more mutations from an Omicron variant. In some embodiments, a plurality of mRNA compositions given in BD₁, BD₂, BD₃, and/or BDₙ are separately administered to a subject, for example, in some embodiments via intramuscular injection, at different injection sites. For example, in some embodiments, a first and second mRNA composition given in BD₁, BD₂, BD₃, and/or BDₙ are separately administered to different arms of a subject via intramuscular injection.

In some embodiments, the length of time between BD₁ and BD₂ (BI₁) is at least about 1 week, at least about 2 weeks, at least about 3 weeks, or at least about 4 weeks. In some embodiments, BI₁ is about 1 week to about 12 weeks. In some embodiments, BI₁ is about 1 week to about 10 weeks. In some embodiments, BI₁ is about 2 weeks to about 10 weeks. In some embodiments, BI₁ is about 2 weeks to about 8 weeks. In some embodiments, BI₁ is about 3 weeks to about 8 weeks. In some embodiments, BI₁ is about 4 weeks to about 8 weeks. In some embodiments, BI₁ is about 6 weeks to about 8 weeks. In some embodiments BI₁ is about 3 to about 4 weeks. In some embodiments, BI₁ is about 1 week. In some embodiments, BI₁ is about 2 weeks. In some embodiments, BI₁ is about 3 weeks. In some embodiments, BI₁ is about 4 weeks. In some embodiments, BI₁ is about 5 weeks. In some embodiments, BI₁ is about 6 weeks. In some embodiments, BI₁ is about 7 weeks. In some embodiments, BI₁ is about 8 weeks. In some embodiments, BI₁ is about 9 weeks. In some embodiments, BI₁ is about 10 weeks.

In some embodiments, the length of time between BD₂ and BD₃ (BI₂) is at least about 1 week, at least about 2 weeks, or at least about 3 weeks. In some embodiments, BI₂ is about 1 week to about 12 weeks. In some embodiments, BI₂ is about 1 week to about 10 weeks. In some embodiments, BI₂ is about 2 weeks to about 10 weeks. In some embodiments, BI₂ is about 2 weeks to about 8 weeks. In some embodiments, BI₂ is about 3 weeks to about 8 weeks. In some embodiments, BI₂ is about 4 weeks to about 8 weeks. In some embodiments, BI₂ is about 6 weeks to about 8 weeks. In some embodiments BI₂ is about 3 to about 4 weeks. In some embodiments, BI₂ is about 1 week. In some embodiments, BI₂ is about 2 weeks. In some embodiments, BI₂ is about 3 weeks. In some embodiments, BI₂ is about 4 weeks. In some embodiments, BI₂ is about 5 weeks. In some embodiments, BI₂ is about 6 weeks. In some embodiments, BI₂ is about 7 weeks. In some embodiments, BI₂ is about 8 weeks. In some embodiments, BI₂ is about 9 weeks. In some embodiments, BI₂ is about 10 weeks.

In some embodiments, the length of time between BD₃ and a subsequent dose that is part of the Booster Dosing Regimen, or between doses for any dose beyond BD₃ (BIₙ) is each separately and independently selected from: about 1 week or more, about 2 weeks or more, or about 3 weeks or more. In some embodiments, BIₙ is about 1 week to about 12 weeks. In some embodiments, BIₙ is about 1 week to about 10 weeks. In some embodiments, BIₙ is about 2 weeks to about 10 weeks. In some embodiments, BIₙ is about 2 weeks to about 8 weeks. In some embodiments, BIₙ is about 3 weeks to about 8 weeks. In some embodiments, BIₙ is about 4 weeks to about 8 weeks. In some embodiments, BIₙ is about 6 weeks to about 8 weeks. In some embodiments BIₙ is about 3 to about 4 weeks. In some embodiments, BIₙ is about 1 week. In some embodiments, BIₙ is about 2 weeks. In some embodiments, BIₙ is about 3 weeks. In some embodiments, BIₙ is about 4 weeks. In some embodiments, BIₙ is about 5 weeks. In some embodiments, BIₙ is about 6 weeks. In some embodiments, BIₙ is about 7 weeks. In some embodiments, BIₙ is about 8 weeks. In some embodiments, BIₙ is about 9 weeks. In some embodiments, BIₙ is about 10 weeks.

In some embodiments, one or more compositions adminstered in BD₁ are formulated in a Tris buffer. In some embodiments, one or more compositions administered in BD₂ are formulated in a Tris buffer. In some embodiments, one or more compositions administering in BD₃ are formulated in a Tris buffer. In some embodiments, one or more compositions administered in BD₃ are formulated in a Tris buffer.

In some embodiments, the Booster dosing regimen comprises administering two or more mRNA compositions described herein, and at least two of the mRNA compositions have different formulations. In some embodiments, the Booster dosing regimen comprises BD₁ and BD₂, where BD₁ comprises administering an mRNA formulated in a Tris buffer and BD₂ comprises administering an mRNA formulated in a PBS buffer. In some embodiments, the Booster dosing regimen comprises BD₁ and BD₂, where BD₁ comprises administering an mRNA formulated in a PBS buffer and BD₂ comprises administering an mRNA formulated in a Tris buffer.

In some embodiments, one or more mRNA compositions given in BD₁, BD₂, BD₃, and/or BDₙ can be administered in combination with another vaccine. In some embodiments, another vaccine is for a disease that is not COVID-19.

In some embodiments, the disease is one that increases deleterious effects of SARS-CoV-2 when a subject is coinfected with the disease and SARS-CoV-2. In some embodiments, the disease is one that increases the transmission rate of SARS-CoV-2 when a subject is coinfected with the disease and SARS-CoV-2. In some embodiments, another vaccine is a different commerically available vaccine. In some embodiments, the different commercially available vaccine is an RNA vaccine. In some embodiments, the different commercially available vaccine is a polypeptide-based vaccine. In some embodiments, another vaccine (e.g., as described herein) and one or more mRNA compositions given in BD₁, BD₂, BD₃, and/or BDₙ are separately administered, for example, in some embodiments via intramuscular injection, at different injection sites. For example, in some embodiments, an influenza vaccine and one or more SARS-CoV-2 mRNA compositions described herein given in BD₁, BD₂, BD₃, and/or BDₙ are separately administered to different arms of a subject via intramuscular injection.

### Exemplary Additional Booster Regimens

In some embodiments, methods of vaccination disclosed herein comprise administering more than one Booster Dosing Regimen. In some embodiments, more than one Booster Dosing Regimen may need to be administered to increase neutralizing antibody response. In some embodiments, more than one booster dosing regimen may be needed to counteract a SARS-CoV-2 strain that has been shown to have a high likelihood of evading immune response elicited by vaccines that a patient has previously received. In some embodiments, an additional Booster Dosing Regimen is administered to a patient who has been determined to produce low concentrations of neutralizing antibodies. In some embodiments, an additional booster dosing regimen is administered to a patient who has been determined to have a high likelihood of being susceptible to SARS-CoV-2 infection, despite previous vaccination (e.g., an immunocompromised patient, a cancer patient, and/or an organ transplant patient).

The description provided above for the first Booster Dosing Regimen also describes the one or more additional Booster Dosing Regimens. The interval of time between the first Booster Dosing Regimen and a second Booster Dosing Regimen, or between subsequent Booster Dosing Regimens can be any of the acceptable intervals of time described above between the Primary Dosing Regimen and the First Booster Dosing Regimen.

In some embodiments, a dosing regimen comprises a primary regimen and a booster regimen, wherein at least one dose given in the primary regimen and/or the booster regimen comprises a composition comprising an RNA that encodes a SARS-CoV-2 S protein or an immunogenic fragment thereof from a SARS-CoV-2 variant that is prevalent or is spreading rapidly in a relevant jurisdiction (e.g., an Omicron variant as described herein). For example, in some embodiments, a primary regimen comprises at least 2 doses of BNT162b2 (e.g., encoding a Wuhan strain), for example, given at least 3 weeks apart, and a booster regimen comprises at least 1 dose of a composition comprising RNA that encodes a S protein or immunogenic fragment thereof from a variant that is prevalent or is spreading rapidly in a relevant jurisdiction (e.g., Omicron variant as described herein). For example, in some embodiments, a primary regimen comprises at least 2 doses of BNT162b2 (e.g., encoding a Wuhan strain), for example, given at least 3 weeks apart, and a booster regimen comprises at least 1 dose of a composition comprising RNA that encodes a S protein or immunogenic fragment thereof from a variant that is prevalent or is spreading rapidly in a relevant jurisdiction (e.g., Omicron variant as described herein). In some such embodiments, such a dose of a booster regimen may further comprise an RNA that encodes a S protein or immunogenic fragment thereof from a Wuhan strain, which can be administered with an RNA that encodes a S protein or immunogenic fragment thereof from a variant that is prevalent or is spreading rapidly in a relevant jurisdiction (e.g., Omicron variant as described herein), as a single mixture, or as two separate compositions, for example, in 1:1 weight ratio. In some embodiments, a booster regimen can also comprise at least 1 dose of BNT162b2, which can be administered as a first booster dose or a subsequent booster dose.

In some embodiments, an RNA composition described herein is given as a booster at a dose that is higher than the doses given during a primary regimen (primary doses) and/or the dose given for a first booster, if any. For example, in some embodiments, such a dose may be 60 ug; or in some embodiments such a dose may be higher than 30 ug and lower than 60 ug (e.g., 55 ug, 50 ug, or lower). In some embodiments, an RNA composition described herein is given as a booster at least 3-12 months or 4-12 months, or 5-12 months, or 6-12 months after the last dose (e.g., the last dose of a primary regimen or a first dose of a booster regimen). In some embodiments, the primary doses and/or the first booster dose (if any) may comprise BNT162b2, for example at 30 ug per dose.

In some embodiments, an RNA composition described herein comprises an RNA encoding a polypeptide as set forth in SEQ ID NO: 49 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 49). In some embodiments, an RNA composition comprises an RNA that includes the sequence of SEQ ID NO: 50 or a variant thereof (e.g., having at least 70% or more, including, e.g., at least 80%, at least 85%, at least 90%, at least 95%, at least 96%, at least 97%, at least 98%, or higher, identity to SEQ ID NO: 50). In some embodiments, an RNA composition comprises an RNA that includes the sequence of SEQ ID NO: 51 or a variant thereof (e.g., having at least 70% or more, including, e.g., at least 80%, at least 85%, at least 90%, at least 95%, at least 96%, at least 97%, at least 98%, or higher, identity to SEQ ID NO: 51).

In some embodiments, an RNA composition described herein comprises an RNA encoding a polypeptide as set forth in SEQ ID NO: 55 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 55. In some embodiments, an RNA composition comprises an RNA that includes the sequence of SEQ ID NO: 56 or a variant thereof (e.g., having at least 70% or more, including, e.g., at least 80%, at least 85%, at least 90%, at least 95%, at least 96%, at least 97%, at least 98%, or higher, identity to SEQ ID NO: 56). In some embodiments, an RNA composition comprises an RNA that includes the sequence of SEQ ID NO: 57 or a variant thereof (e.g., having at least 70% or more, including, e.g., at least 80%, at least 85%, at least 90%, at least 95%, at least 96%, at least 97%, at least 98%, or higher, identity to SEQ ID NO: 57).

In some embodiments, an RNA composition described herein comprises an RNA encoding a polypeptide as set forth in SEQ ID NO: 58 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 58). In some embodiments, an RNA composition comprises an RNA that includes the sequence of SEQ ID NO: 59 or a variant thereof (e.g., having at least 70% or more, including, e.g., at least 80%, at least 85%, at least 90%, at least 95%, at least 96%, at least 97%, at least 98%, or higher, identity to SEQ ID NO: 59). In some embodiments, an RNA composition comprises an RNA that includes the sequence of SEQ ID NO: 60 or a variant thereof (e.g., having at least 70% or more, including, e.g., at least 80%, at least 85%, at least 90%, at least 95%, at least 96%, at least 97%, at least 98%, or higher, identity to SEQ ID NO: 60).

In some embodiments, an RNA composition described herein comprises an RNA encoding a polypeptide as set forth in SEQ ID NO: 61 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 61). In some embodiments, an RNA composition comprises an RNA that includes the sequence of SEQ ID NO: 62 or a variant thereof (e.g., having at least 70% or more, including, e.g., at least 80%, at least 85%, at least 90%, at least 95%, at least 96%, at least 97%, at least 98%, or higher, identity to SEQ ID NO: 62). In some embodiments, an RNA composition comprises an RNA that includes the sequence of SEQ ID NO: 63 or a variant thereof (e.g., having at least 70% or more, including, e.g., at least 80%, at least 85%, at least 90%, at least 95%, at least 96%, at least 97%, at least 98%, or higher, identity to SEQ ID NO: 63).

In some embodiments, an RNA composition described herein comprises an RNA encoding a polypeptide as set forth in SEQ ID NO: 129 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 129). In some embodiments, an RNA composition comprises an RNA that includes the sequence of SEQ ID NO: 130 or a variant thereof (e.g., having at least 70% or more, including, e.g., at least 80%, at least 85%, at least 90%, at least 95%, at least 96%, at least 97%, at least 98%, or higher, identity to SEQ ID NO: 130). In some embodiments, an RNA composition comprises an RNA that includes the sequence of SEQ ID NO: 132 or a variant thereof (e.g., having at least 70% or more, including, e.g., at least 80%, at least 85%, at least 90%, at least 95%, at least 96%, at least 97%, at least 98%, or higher, identity to SEQ ID NO: 132).

In some embodiments, an RNA composition described herein (e.g., a monovalent, bivalent, trivalent, or quadrivalent composition) comprises an RNA encoding a polypeptide as set forth in SEQ ID NO: 134 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 134). In some embodiments, an RNA composition comprises an RNA that includes the sequence of SEQ ID NO: 135 or a variant thereof (e.g., having at least 70% or more, including, e.g., at least 80%, at least 85%, at least 90%, at least 95%, at least 96%, at least 97%, at least 98%, or higher, identity to SEQ ID NO: 135). In some embodiments, an RNA composition comprises an RNA that includes the sequence of SEQ ID NO: 137 or a variant thereof (e.g., having at least 70% or more, including, e.g., at least 80%, at least 85%, at least 90%, at least 95%, at least 96%, at least 97%, at least 98%, or higher, identity to SEQ ID NO: 137).

In some embodiments, an RNA composition described herein (e.g., a monovalent, bivalent, trivalent, or quadrivalent composition) comprises an RNA encoding a polypeptide as set forth in SEQ ID NO: 139 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 139). In some embodiments, an RNA composition comprises an RNA that includes the sequence of SEQ ID NO: 140 or a variant thereof (e.g., having at least 70% or more, including, e.g., at least 80%, at least 85%, at least 90%, at least 95%, at least 96%, at least 97%, at least 98%, or higher, identity to SEQ ID NO: 140). In some embodiments, an RNA composition comprises an RNA that includes the sequence of SEQ ID NO: 142 or a variant thereof (e.g., having at least 70% or more, including, e.g., at least 80%, at least 85%, at least 90%, at least 95%, at least 96%, at least 97%, at least 98%, or higher, identity to SEQ ID NO: 142).

In some embodiments, an RNA composition described herein (e.g., a monovalent, bivalent, trivalent, or quadrivalent composition) comprises an RNA encoding a polypeptide as set forth in SEQ ID NO: 144 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 144). In some embodiments, an RNA composition comprises an RNA that includes the sequence of SEQ ID NO: 145 or a variant thereof (e.g., having at least 70% or more, including, e.g., at least 80%, at least 85%, at least 90%, at least 95%, at least 96%, at least 97%, at least 98%, or higher, identity to SEQ ID NO: 145). In some embodiments, an RNA composition comprises an RNA that includes the sequence of SEQ ID NO: 147 or a variant thereof (e.g., having at least 70% or more, including, e.g., at least 80%, at least 85%, at least 90%, at least 95%, at least 96%, at least 97%, at least 98%, or higher, identity to SEQ ID NO: 147).

In some embodiments, the formulations disclosed herein can be used to carry out any of the dosing regimens described in Table 27 (below).

TABLE 27
Exemplary Dosing Regimens:

| | | | | | | Time between the last | | | | |
|---|---|---|---|---|---|---|---|---|---|---|
| | Primary Regimen | | | | | dose of a Primary | Booster Regimen | | | |
| | | | | | Dose 1 | regimen and a first | | | | Dose 1 |
| | Dose 1 | Dose 2 | Time Between | | and Dose 2 | dose of Booster | Dose 1 | Dose 2 | Time Between | and Dose 2 |
| # | (μg RNA) | (μg RNA) | Doses 1 and 2 | | Formulation | Regimen | (μg RNA) | (μg RNA) | Doses 1 and 2 | Formulation |
| 1 | 30 | 30 | 2 to 8 weeks | | PBS | At least 2 months | 30 | N/A¹ | N/A | PBS |
| 2 | 30 | 30 | 2 to 8 weeks | | PBS | At least 3 months | 30 | N/A¹ | N/A | PBS |
| 3 | 30 | 30 | 2 to 8 weeks | | PBS | 6 to 12 months | 30 | N/A¹ | N/A | PBS |
| 4 | 30 | 30 | 2 to 8 weeks | | PBS or Tris | 4 to 12 months | 15 | N/A¹ | N/A | PBS or Tris |
| 5 | 30 | 30 | 2 to 8 weeks | | PBS or Tris | 4 to 12 months | 10 | N/A¹ | N/A | PBS or Tris |
| 6 | 30 | 30 | 2 to 8 weeks | | PBS or Tris | 4 to 12 months | 30 | 30 | 4 to 12 months | PBS or Tris |
| 7 | 30 | 30 | 2 to 8 weeks | | PBS or Tris | 4 to 12 months | 30 | 15 | 4 to 12 months | PBS or Tris |
| 8 | 30 | 30 | 2 to 8 weeks | | PBS or Tris | 4 to 12 months | 30 | 10 | 4 to 12 months | PBS or Tris |
| 9 | 30 | 30 | 2 to 8 weeks | | PBS or Tris | 4 to 12 months | 30 | 60 | 4 to 12 months | PBS or Tris |
| 10 | 30 | 30 | 2 to 8 weeks | | PBS or Tris | 4 to 12 months | 30 | >30 to <60 | 4 to 12 months | PBS or Tris |
| 11 | 30 | 30 | 2 to 8 weeks | | PBS or Tris | 4 to 12 months | 30 | 50 | 4 to 12 months | PBS or Tris |
| 12 | 30 | 30 | 2 to 8 weeks | | PBS | At least 6 months | 30 | N/A¹ | N/A | PBS |
| 13 | 30 | 30 | ~21 | days | PBS | At least 2 months | 30 | N/A¹ | N/A | PBS |
| 14 | 30 | 30 | ~21 | days | PBS | At least 3 months | 30 | N/A¹ | N/A | PBS |
| 15 | 30 | 30 | ~21 | days | PBS | 6 to 12 months | 30 | N/A¹ | N/A | PBS |
| 16 | 30 | 30 | ~21 | days | PBS | At least 6 months | 30 | N/A¹ | N/A | PBS |
| 17 | 30 | 30 | 21 | days | PBS | At least 6 months | 15 | 15 | ~21 days | PBS |
| 18 | 30 | 30 | 21 | days | PBS | At least 6 months | 15 | 15 | ~21 days | PBS |
| 19 | 30 | 30 | 2 to 8 weeks | | PBS | At least 2 months | 30 | N/A¹ | N/A | Tris |
| 20 | 30 | 30 | 2 to 8 weeks | | PBS | At least 3 months | 30 | N/A¹ | N/A | Tris |
| 21 | 30 | 30 | 2 to 8 weeks | | PBS | 6 to 12 months | 30 | N/A¹ | N/A | Tris |
| 22 | 30 | 30 | 2 to 8 weeks | | PBS | At least 6 months | 30 | N/A¹ | N/A | Tris |
| 23 | 30 | 30 | ~21 | days | PBS | At least 2 months | 30 | N/A¹ | N/A | Tris |
| 24 | 30 | 30 | ~21 | days | PBS | At least 3 months | 30 | N/A¹ | N/A | Tris |
| 25 | 30 | 30 | ~21 | days | PBS | 6 to 12 months | 30 | N/A¹ | N/A | Tris |
| 26 | 30 | 30 | ~21 | days | PBS | At least 6 months | 30 | N/A¹ | N/A | Tris |
| 27 | 30 | 30 | 21 | days | PBS | At least 6 months | 15 | 15 | ~21 days | Tris |
| 28 | 30 | 30 | 21 | days | PBS | At least 6 months | 15 | 15 | ~21 days | Tris |
| 29 | 30 | 30 | 2 to 8 weeks | | Tris | At least 2 months | 30 | N/A¹ | N/A | Tris |
| 30 | 30 | 30 | 2 to 8 weeks | | Tris | At least 3 months | 30 | N/A¹ | N/A | Tris |
| 31 | 30 | 30 | 2 to 8 weeks | | Tris | 6 to 12 months | 30 | N/A¹ | N/A | Tris |
| 32 | 30 | 30 | 2 to 8 weeks | | Tris | At least 6 months | 30 | N/A¹ | N/A | Tris |
| 33 | 30 | 30 | ~21 | days | Tris | At least 2 months | 30 | N/A¹ | N/A | Tris |
| 34 | 30 | 30 | ~21 | days | Tris | At least 3 months | 30 | N/A¹ | N/A | Tris |
| 35 | 30 | 30 | ~21 | days | Tris | 6 to 12 months | 30 | N/A¹ | N/A | Tris |
| 36 | 30 | 30 | ~21 | days | Tris | At least 6 months | 30 | N/A¹ | N/A | Tris |
| 37 | 30 | 30 | 21 | days | Tris | At least 6 months | 15 | 15 | ~21 days | Tris |
| 38 | 30 | 30 | 21 | days | Tris | At least 6 months | 15 | 15 | ~21 days | Tris |
| 39 | 10 | 10 | 2 to 8 weeks | | Tris | At least 2 months | 10 | N/A¹ | N/A | Tris |
| 40 | 10 | 10 | 2 to 8 weeks | | Tris | At least 3 months | 10 | N/A¹ | N/A | Tris |
| 41 | 10 | 10 | 2 to 8 weeks | | Tris | 6 to 12 months | 10 | N/A¹ | N/A | Tris |
| 42 | 10 | 10 | 2 to 8 weeks | | Tris | At least 6 months | 10 | N/A¹ | N/A | Tris |
| 43 | 10 | 10 | ~21 | days | Tris | At least 2 months | 10 | N/A¹ | N/A | Tris |
| 44 | 10 | 10 | ~21 | days | Tris | At least 3 months | 10 | N/A¹ | N/A | Tris |
| 45 | 10 | 10 | ~21 | days | Tris | 6 to 12 months | 10 | N/A¹ | N/A | Tris |
| 46 | 10 | 10 | ~21 | days | Tris | At least 6 months | 10 | N/A¹ | N/A | Tris |
| 47 | 3 | 3 | 2 to 8 weeks | | Tris | At least 2 months | 3 | N/A¹ | N/A | Tris |
| 48 | 3 | 3 | 2 to 8 weeks | | Tris | At least 3 months | 3 | N/A¹ | N/A | Tris |
| 49 | 3 | 3 | 2 to 8 weeks | | Tris | 6 to 12 months | 3 | N/A¹ | N/A | Tris |
| 50 | 3 | 3 | 2 to 8 weeks | | Tris | At least 6 months | 3 | N/A¹ | N/A | Tris |
| 51 | 3 | 3 | ~21 | days | Tris | At least 2 months | 3 | N/A¹ | N/A | Tris |
| 52 | 3 | 3 | ~21 | days | Tris | At least 3 months | 3 | N/A¹ | N/A | Tris |
| 53 | 3 | 3 | ~21 | days | Tris | 6 to 12 months | 3 | N/A¹ | N/A | Tris |
| 54 | 3 | 3 | ~21 | days | Tris | At least 6 months | 3 | N/A¹ | N/A | Tris |

¹N/A refers to no dose necessary.

In some embodiments of certain exemplary dosing regimens as described in Table 27 above, an RNA composition described herein (e.g., comprising RNA encoding a variant described herein) is given in a first dose of a primary regimen. In some embodiments of certain exemplary dosing regimens as described in Table 27 above, an RNA composition described herein (e.g., comprising RNA encoding a variant described herein) is given in a second dose of a primary regimen. In some embodiments of certain exemplary dosing regimens as described in Table 27 above, an RNA composition described herein (e.g., comprising RNA encoding a variant described herein) is given in a first dose and a second dose of a primary regimen. In some embodiments of certain exemplary dosing regimens as described in Table 27 above, an RNA composition described herein (e.g., comprising RNA encoding a variant described herein) is given in a first dose of a booster regimen. In some embodiments of certain exemplary dosing regimens as described in Table 27 above, an RNA composition described herein (e.g., comprising RNA encoding a variant described herein) is given in a second dose of a booster regimen. In some embodiments of certain exemplary dosing regimens as described in Table 27 above, an RNA composition described herein (e.g., comprising RNA encoding a variant described herein) is given in a first dose and a second dose of a booster regimen. In some embodiments of certain exemplary dosing regimens as described in Table 27 above, an RNA composition described herein (e.g., comprising RNA encoding a variant described herein) is given in a first dose and a second dose of a primary regimen and also in at least one dose of a booster regimen. In some embodiments of certain exemplary dosing regimens as described in Table 27 above, an RNA composition described herein (e.g., comprising RNA encoding a variant described herein) is given in at least one dose (including, e.g., at least two doses) of a booster regimen and BNT162b2 is given in a primary regimen. In some embodiments of certain exemplary dosing regimens as described in Table 27 above, an RNA composition described herein (e.g., comprising RNA encoding a variant described herein) is given in a second dose of a booster regimen and BNT162b2 is given in a primary regimen and in a first dose of a booster regimen. In some embodiments, an RNA composition described herein (e.g., comprising RNA encoding a variant described herein) comprises an RNA encoding a polypeptide as set forth in SEQ ID NO: 49 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 49). In some embodiments, an RNA composition described herein (e.g., comprising RNA encoding a variant described herein) comprises an RNA that includes the sequence of SEQ ID NO: 50 or a variant thereof (e.g., having at least 70% or more, including, e.g., at least 80%, at least 85%, at least 90%, at least 95%, at least 96%, at least 97%, at least 98%, or higher, identity to SEQ ID NO: 50). In some embodiments, an RNA composition described herein (e.g., comprising RNA encoding a variant described herein) comprises an RNA that includes the sequence of SEQ ID NO: 51 or a variant thereof (e.g., having at least 70% or more, including, e.g., at least 80%, at least 85%, at least 90%, at least 95%, at least 96%, at least 97%, at least 98%, or higher, identity to SEQ ID NO: 51).

In some embodiments, an RNA composition described herein comprises an RNA encoding a polypeptide as set forth in SEQ ID NO: 55 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 55). In some embodiments, an RNA composition comprises an RNA that includes the sequence of SEQ ID NO: 56 or a variant thereof (e.g., having at least 70% or more, including, e.g., at least 80%, at least 85%, at least 90%, at least 95%, at least 96%, at least 97%, at least 98%, or higher, identity to SEQ ID NO: 56). In some embodiments, an RNA composition comprises an RNA that includes the sequence of SEQ ID NO: 57 or a variant thereof (e.g., having at least 70% or more, including, e.g., at least 80%, at least 85%, at least 90%, at least 95%, at least 96%, at least 97%, at least 98%, or higher, identity to SEQ ID NO: 57).

In some embodiments, an RNA composition described herein comprises an RNA encoding a polypeptide as set forth in SEQ ID NO: 58 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 58). In some embodiments, an RNA composition comprises an RNA that includes the sequence of SEQ ID NO: 59 or a variant thereof (e.g., having at least 70% or more, including, e.g., at least 80%, at least 85%, at least 90%, at least 95%, at least 96%, at least 97%, at least 98%, or higher, identity to SEQ ID NO: 59). In some embodiments, an RNA composition comprises an RNA that includes the sequence of SEQ ID NO: 60 or a variant thereof (e.g., having at least 70% or more, including, e.g., at least 80%, at least 85%, at least 90%, at least 95%, at least 96%, at least 97%, at least 98%, or higher, identity to SEQ ID NO: 60).

In some embodiments, an RNA composition described herein comprises an RNA encoding a polypeptide as set forth in SEQ ID NO: 61 or an immunogenic fragment thereof, or a variant thereof (e.g., having at least 70% or more, including, e.g., at least 80%, at least 85%, at least 90%, at least 95%, at least 96%, at least 97%, at least 98%, or higher, identity to SEQ ID NO: 61). In some embodiments, an RNA composition comprises an RNA that includes the sequence of SEQ ID NO: 62 or a variant thereof (e.g., having at least 70% or more, including, e.g., at least 80%, at least 85%, at least 90%, at least 95%, at least 96%, at least 97%, at least 98%, or higher, identity to SEQ ID NO: 62). In some embodiments, an RNA composition comprises an RNA that includes the sequence of SEQ ID NO: 63 or a variant thereof (e.g., having at least 70% or more, including, e.g., at least 80%, at least 85%, at least 90%, at least 95%, at least 96%, at least 97%, at least 98%, or higher, identity to SEQ ID NO: 63).

In some embodiments, such an RNA composition described herein (e.g., comprising RNA encoding a variant described herein) can further comprise RNA encoding a S protein or an immunogenic fragment thereof of a different strain (e.g., a Wuhan strain). By way of example, in some embodiments, a second dose of a booster regimen of Regimens #9-11 as described in Table 27 above can comprise an RNA composition described herein (e.g., comprising RNA encoding a variant described herein such as Omicron, for example, in one embodiment RNA as described in this Example) and a BNT162b2 construct, for example, in 1:1 weight ratio.

In some embodiments of Regimen #6 as described in Table 27 above, a first dose and a second dose of a primary regimen and a first dose and a second dose of a booster regimen each comprise an RNA composition described herein (e.g., comprising RNA encoding a variant described herein such as Omicron, for example, in one embodiment RNA as described in this Example). In some such embodiments, a second dose of a booster regimen may not be necessary.

In some embodiments of Regimen #6 as described in Table 27 above, a first dose and a second dose of a primary regimen and a first dose and a second dose of a booster regimen each comprise an RNA composition described herein (e.g., comprising RNA encoding a variant described herein such as Omicron, for example, in one embodiment RNA as described in this Example). In some such embodiments, a second dose of a booster regimen may not be necessary.

In some embodiments of Regimen #6 as described in Table 27 above, a first dose and a second dose of a primary regimen each comprise a BNT162b2 construct, and a first dose and a second dose of a booster regimen each comprise an RNA composition described herein (e.g., comprising RNA encoding a variant described herein such as Omicron, for example, in one embodiment RNA as described in this Example). In some such embodiments, a second dose of a booster regimen may not be necessary.

In some embodiments of Regimen #6 as described in Table 27 above, a first dose and a second dose of a primary regimen and a first dose of a booster regimen each comprise a BNT162b2 construct, and a second dose of a booster regimen comprises an RNA composition described herein (e.g., comprising RNA encoding a variant described herein such as Omicron, for example, in one embodiment RNA as described in this Example).

Furthermore, the present disclosure provides an insight that, given similarities among S protein sequences of BA.2 and BA.4/5 variants, combining vaccination doses that comprise or deliver BA.4 and/or BA.5 variant spike sequences with those of that comprise or deliver Wuhan spike sequences may also achieve particularly broad immunization (i.e., synergistic immunization as described herein).

In some embodiments, compositions described herein can comprise two or more SARS-CoV-2 antigens, wherein the two or more antigens are from synergistic categories of coronavirus strain and/or variant sequences (e.g., SARS-CoV-2 strain and/or variant sequences). In some embodiments, synergistic categories can be defined based on shared amino acid alterations in the S glycoprotein of a coronavirus strain and/or a coronavirus variant. For example, while many of amino acid changes in the RBD are shared between Omicron sub-lineages (e.g., BA.1, BA.2, BA.2.12.1, and BA.4/5), alterations within the NTD of BA.2 and BA.2-derived sub-lineages including BA.4/5 are mostly distinct from those found in BA.1. Therefore, in some embodiments, synergistic categories of coronavirus strain and/or variant sequences (e.g., SARS-CoV-2 strain and/or variant sequences) can be defined based on the degree of shared amino acid mutations present with the NTD of a S protein. For example, in some embodiments where two SARS-CoV-2 strain and/or variant sequences share at least 50% (including, e.g., at least 60%, at least 70%, at least 80%, at least 90%, or more) of the amino acid mutations present in the NTD of a S protein, both SARS-CoV-2 strain and variant sequences can be grouped into the same category. In some embodiments where two SARS-CoV-2 strain and/or variant sequences share no more 50% (including, e.g., no more than 45%, no more than 40%, no more than 30%, or lower) of the amino acid mutations present in the NTD of a S protein, both SARS-CoV-2 strain and variant sequences can be grouped into different categories. Among other things, the present findings provide insights that exposing subjects (e.g., via infection and/or vaccination) to at least two antigens that are of different synergistic categories (e.g., as shown in the table below) can produce a more robust immune response (e.g., broadening the spectrum of cross-neutralization against different variants and/or producing an immune response that is less prone to immune escape).

| Category I | Category II |
|---|---|
| Wuhan strain | Omicron BA.2 |
| Alpha variant | Omicron BA.2.11.2 |
| Beta variant | Omicron BA.4 |
| Delta variant | Omicron BA.5 |
| Omicron BA.1 | XBB.1.5 |
| Sublineages derived from | BA.2.75.2 |
| any one of the above | |
| | BQ.1.1 |
| | Sublineages derived from |
| | any one of the above |

For example, in some embodiments, vaccine-naïve subjects without prior infection may be administered a combination of vaccines, at least two of which are each adapted to a SARS-CoV-2 strain of different synergistic categories (e.g., as described herein). In some embodiments, such vaccines in a combination may be administered at different times, for example, in some embodiments as a first dose and a second dose administered apart by a pre-determined period of time (e.g., according to certain dosing regimens as described herein). In some embodiments, such vaccines in a combination may be administered as a multivalent vaccine. In some embodiments, subject infected or vaccinated with a SARS-CoV-2 strain of one category may be administered with a vaccine adapted to a SARS-CoV-2 strain of a different category (e.g., as described herein). In some embodiments, such a vaccine may be a polypeptide-based or RNA-based vaccine.

In some embodiments, a dose comprising one or more vaccines of Category II is administered to a subject previously administered one or more vaccines of Category I. In some embodiments, a dose comprising one or more vaccines of Category I and one or more vaccines of Category II is administered to a subject to whom one or more vaccines of Category I was previously administered (e.g., in some embodiments, the dose comprises at least one vaccine that was previously administered to the subject).

In some embodiments, a combination of vaccines comprising a vaccine from Category I and a BA.4/5 vaccine may provide a particularly superior immune response (e.g., an immune response with particularly strong cross-neutralization effects).

Due to the large number of differences between XBB and its variants, in some embodiments, a particularly improved synergistic effect can be produced by a combination comprising a vaccine of Category I and an XBB vaccine. In some embodiments, a particularly improved synergistic effect can be produced by a combination comprising a BA.4/5 vaccine and an XBB vaccine.

While the present findings are based on retrospective analyses of samples derived from different studies, using relatively small samples sizes and cohorts that are not fully aligned regarding immunization intervals and demographic characteristics such as age and sex of individuals, the present findings provide useful insights for vaccine design and vaccination strategies for improving cross-neutralization against a broader spectrum of SARS-COV-2 variants.

In some embodiments, a vaccine can comprise a polypeptide (e.g., a non-natural polypeptide, e.g., a chimeric polypeptide) comprising one or more mutations that are characteristic of one or more different SARS-CoV-2 variants, or a nucleic acid (e.g., in some embodiments an RNA) encoding the polypeptide. In some embodiments, a vaccine can comprise a polypeptide (e.g., a non-natural polypeptide, e.g., a chimeric polypeptide) comprising one or more mutations that are characteristic of a first SARS-CoV-2 variant and one or more mutations that are characteristic of a second SARS-CoV-2 variant, or a nucleic acid (e.g., in some embodiments an RNA) encoding the polypeptide. In some embodiments, a first SARS-CoV-2 variant can be a SARS-CoV-2 strain/variant from Category I of the table above, while a second SARS-CoV-2 variant can be a SARS-CoV-2 strain/variant from Category II of the table above. For example, in some embodiments, a vaccine can comprise a polypeptide that comprises an RBD comprising one or more mutations that are characteristic of a first SARS-CoV-2 variant and an NTD comprising one or more mutations that are characteristic of a second SARS-CoV-2 variant, or a nucleic acid (e.g., in some embodiments an RNA) encoding the polypeptide.

In some embodiments, a vaccine can comprise a polypeptide that comprises an RBD comprising one or more mutations characteristic of a BA.1 Omicron variant and an NTD comprising one or more mutations characteristic of a second SARS-CoV-2 variant that is not a BA.1 Omicron variant, or a nucleic acid (e.g., in some embodiments an RNA) encoding the polypeptide. In some embodiments, a vaccine can comprise or encode a polypeptide comprising an NTD comprising one or more mutations characteristic of a BA.1 Omicron variant and an RBD comprising one or more mutations characteristic of a second SARS-CoV-2 variant that is not a BA.1 Omicron variant. In some embodiments, a vaccine can comprise a polypeptide that comprises an RBD comprising one or more mutations characteristic of a BA.1 Omicron variant and an NTD comprising one or more mutations characteristic of a BA.2 Omicron variant, or a nucleic acid (e.g., in some embodiments an RNA) encoding the polypeptide. In some embodiments, a vaccine can comprise a polypeptide that comprises an RBD comprising one or more mutations characteristic of a BA.1 Omicron variant and an NTD comprising one or more mutations characteristic of a BA.4/5 Omicron variant, or a nucleic acid (e.g., in some embodiments an RNA) encoding the polypeptide.

In some embodiments, a vaccine can comprise a polypeptide that comprises an NTD comprising one or more mutations characteristic of a BA.1 Omicron variant and an RBD comprising one or more mutations characteristic of a second SARS-CoV-2 variant that is not a BA.1 Omicron variant, or a nucleic acid (e.g., in some embodiments an RNA) encoding the polypeptide. In some embodiments, a vaccine can comprise a polypeptide that comprises an RBD comprising one or more mutations characteristic of a BA.1 Omicron variant and an NTD comprising one or more mutations characteristic of a second SARS-CoV-2 variant that is not a BA.1 Omicron variant, or a nucleic acid (e.g., in some embodiments an RNA) encoding the polypeptide. In some embodiments, a vaccine can comprise a polypeptide that comprises an NTD comprising one or more mutations characteristic of a BA.1 Omicron variant and an RBD comprising one or more mutations characteristic of a BA.2 Omicron variant, or a nucleic acid (e.g., in some embodiments an RNA) encoding the polypeptide. In some embodiments, a vaccine can comprise a polypeptide that comprises one or more mutations characteristic of an NTD of a BA.1 Omicron variant and an RBD comprising one or more mutations characteristic of a BA.4/5 Omicron variant, or a nucleic acid (e.g., in some embodiments an RNA) encoding the polypeptide.

In some embodiments, a vaccine can comprise a polypeptide that comprises an RBD comprising one or more mutations characteristic of a BA.1 Omicron variant and an NTD of a Wuhan S protein, or a nucleic acid (e.g., in some embodiments an RNA) encoding the polypeptide. In some embodiments, a vaccine can comprise a polypeptide that comprises an RBD comprising one or more mutations characteristic of a BA.2 Omicron variant and an NTD of a Wuhan S protein, or a nucleic acid (e.g., in some embodiments an RNA) encoding the polypeptide. In some embodiments, a vaccine can comprise a polypeptide that comprises an RBD comprising one or more mutations characteristic of a BA.4/5 Omicron variant and an NTD of a Wuhan S protein, or a nucleic acid (e.g., in some embodiments an RNA) encoding the polypeptide.

In some embodiments, a vaccine can comprise a polypeptide that comprises an NTD comprising one or more mutations characteristic of a BA.1 Omicron variant and an RBD of a Wuhan S protein, or a nucleic acid (e.g., in some embodiments an RNA) encoding the polypeptide. In some embodiments, a vaccine can comprise a polypeptide that comprises an NTD comprising one or more mutations characteristic of a BA.2 Omicron variant and an RBD of a Wuhan S protein, or a nucleic acid (e.g., in some embodiments an RNA) encoding the polypeptide. In some embodiments, a vaccine can comprise a polypeptide that comprises an NTD comprising one or more mutations characteristic of a BA.4/5 Omicron variant and an RBD of a Wuhan S protein, or a nucleic acid (e.g., in some embodiments an RNA) encoding the polypeptide.

In some embodiments, an RNA composition described herein (e.g., an RNA composition comprising (a) one or more RNAs, each encoding an S protein of a different SARS-CoV-2 strain or variant, or an immunogenic fragment thereof, and (b) (i) one or more RNAs, each encoding an antigen of a different influenza strain or (ii) a commercial influenza vaccine (e.g., a commercial vaccine described herein) comprises RNA(s) encoding an S protein of a SARS-CoV-2 strain or variant or a combination of SARS-CoV-2 strains/variants listed in the below Table D.

TABLE D
S proteins of SARS-CoV-2 Variant or Strains, or Strain/Variant
Combinations for Delivering in Combination Vaccines

| | Variant or Strain Combination of |
|---|---|
| | Variants/Strains |
| 1 | Wuhan + Omicron BA.4/5 |
| 2 | Wuhan + XBB.1.5 |
| 3 | Wuhan + BQ.1.1 |
| 4 | Wuhan + BA.2.75.2 |
| 5 | XBB.1.5 |
| 6 | XBB.1.5 + BA.4/5 |
| 7 | XBB.1.5 + BA.2.75.2 |
| 8 | XBB.1.5 + BQ.1.1 |
| 9 | BA.2.75.2 |
| 10 | BA.2.75.2 + BQ.1.1 |
| 11 | BQ.1.1 |
| 12 | Wuhan + BA.4/5 + XBB.1.5 |
| 13 | Wuhan + BA.4/5 + BQ.1.1 |
| 14 | Wuhan + BA.4/5 + BA.2.75.2 |
| 15 | BA.4/5 + BA.2.75.2 |
| 16 | BA.4/5 + BQ.1.1 |
| 17 | XBB.1.16 |
| 18 | XBB.1.16 + BA.4/5 |
| 19 | XBB.1.16 + Wuhan |
| 20 | XBB.2.3 |
| 21 | XBB.2.3 + BA.4/5 |
| 22 | XBB.2.3 + Wuhan |
| 23 | XBB.2.3.2 |
| 24 | XBB.2.3.2 + BA.4/5 |
| 25 | XBB.2.3.2 + Wuhan |

In some embodiments, an RNA composition comprising RNA(s) encoding a SARS-CoV-2 S protein of a strain or variant listed in Table D, or a SARS-CoV-2 S protein of each strain/variant in a combination listed in Table D can produce an improved immune response (e.g., an immune response with improved cross-neutralization and/or higher neutralization titers against one or more SARS-CoV-2 variants of concern), e.g., relative to an RNA composition comprising an RNA encoding a SARS-CoV-2 S protein of a Wuhan strain.

In some embodiments, an RNA composition comprising RNA(s) encoding a SARS-CoV-2 S protein of a strain or variant listed in Table D, or a SARS-CoV-2 S protein of each strain/variant in a combination listed in Table D is administered to a subject who has not previously received a SARS-CoV-2 vaccine (e.g., as a first dose and/or as part of a priming vaccination regimen (e.g., (i) two doses of such a composition, administered approximately 21 days apart, or (ii) three doses of such a composition, where the first and the second doses are administered approximately 21 days apart and the second and the third dose are administered about 28 days apart).

In some embodiments, an RNA composition that comprises RNA(s) encoding a SARS-CoV-2 S protein of a strain or variant listed in Table D, or a SARS-CoV-2 S protein of each strain/variant in a combination listed in Table D is administered to a subject who has previously been exposed to a SARS-CoV-2 antigen (e.g., a subject who has previously received at least one dose (e.g., a complete dosing regimen) of a SARS-CoV-2 vaccine and/or previously been infected one or more times with SARS-CoV-2). In some embodiments, an RNA composition that comprises RNA(s) encoding one or more S protein(s) of a variant or combination of strain/variants listed in Table D is administered as a booster dose.

In some embodiments, an RNA composition comprising RNA(s) that encodes a SARS-CoV-2 S protein of a strain/variant listed in Table D, or a SARS-CoV-2 S protein of each strain/variant in a combination listed in Table D is administered to a subject previously administered one or more doses of an RNA composition that delivers a SARS-CoV-2 S protein of a Wuhan strain. In some embodiments, an RNA composition that comprises RNA(s) that encode a SARS-CoV-2 S protein of a strain/variant listed in Table D, or a SARS-CoV-2 S protein of each strain/variant in a combination listed in Table D is administered as a booster dose to a subject previously administered a priming dosing regimen of a composition (e.g., an RNA composition) that delivers a SARS-CoV-2 S protein of a Wuhan strain (e.g., a subject previously administered (i) two doses of an RNA vaccine that encodes a SARS-CoV-2 S protein of a Wuhan strain, where the first and the second doses are administered about 21 days apart, or (ii) three doses of an RNA vaccine that encodes a SARS-CoV-2 S protein of a Wuhan strain, where the first and the second dose are administered about 21 days apart and the third dose is administered about 28 days after the second dose). In some embodiments, an RNA composition that comprises RNA(s) encoding a SARS-CoV-2 S protein of a strain/variant listed in Table D, or each strain/variant in a combination listed in Table D is administered as a further booster dose to a subject previously administered a priming dosing regimen and one or more booster doses of a composition (e.g., an RNA composition) that delivers a SARS-CoV-2 S protein of a Wuhan strain (e.g., a subject previously administered (i) three doses of an RNA vaccine that encodes a SARS-CoV-2 S protein of a Wuhan strain, where the first and the second doses are administered about 21 days apart, and the third dose is administered at least about 2 months after the second dose, (ii) four doses of an RNA vaccine that encodes a SARS-CoV-2 S protein of a Wuhan strain, where the first and the second dose are administered about 21 days apart, the third dose is administered about 28 days after the second dose, and the fourth dose is administered at least about three months after the second dose, or (iii) four doses of an RNA vaccine that encodes a SARS-CoV-2 S protein of a Wuhan strain, where the first and the second dose are administered about 21 days apart, the third dose is administered at least about 2 months after the second dose, and the fourth dose is administered at least about two months after the second dose).

In some embodiments, an RNA composition that comprises RNA(s) encoding a SARS-CoV-2 S protein of a strain/variant listed in Table D, or a SARS-CoV-2 S protein of each strain/variant in a combination listed in Table D is administered to a subject previously administered one or more doses of a bivalent composition (e.g., an RNA composition) that delivers a SARS-CoV-2 S protein of a Wuhan strain and a SARS-CoV-2 S protein of an Omicron BA.4/5 variant. In some embodiments, an RNA composition that comprises RNA(s) encoding one or more SARS-CoV-2 S protein(s) of a strain/variant or combination of strain/variants listed in Table D is administered as a booster dose to a subject previously administered a priming dosing regimen of a bivalent composition (e.g., an RNA composition) that delivers a SARS-CoV-2 S protein of a Wuhan strain and a SARS-CoV-2 S protein of an Omicron BA.4/5 variant (e.g., a subject previously administered (i) two doses of a bivalent RNA vaccine, where the first and the second doses are administered about 21 days apart, or (ii) three doses of a bivalent RNA vaccine, where the first and the second dose are administered about 21 days apart and the third dose is administered about 28 days after the second dose). In some embodiments, an RNA composition that comprises RNA(s) encoding a SARS-CoV-2 S protein of a strain/variant listed in Table D, or a SARS-CoV-2 S protein of each strain/variant in a combination listed in Table D is administered as a further booster dose to a subject previously administered a priming dosing regimen and one or more booster doses of a bivalent composition (e.g., an RNA composition) that delivers a SARS-CoV-2 S protein of a Wuhan strain and a SARS-CoV-2 S protein of an Omicron BA.4/5 variant (e.g., a subject previously administered (i) three doses of a bivalent RNA vaccine, where the first and the second doses are administered about 21 days apart, and the third dose is administered at least about 2 months after the second dose, (ii) four doses of a bivalent RNA vaccine, where the first and the second dose are administered about 21 days apart, the third dose is administered about 28 days after the second dose, and the fourth dose is administered at least about three months after the second dose, or (iii) four doses of a bivalent RNA vaccine, where the first and the second dose are administered about 21 days apart, the third dose is administered at least about 2 months after the second dose, and the fourth dose is administered at least about two months after the second dose).

In some embodiments, an RNA composition that comprises RNA(s) encoding a SARS-CoV-2 S protein of a strain/variant listed in Table D, or a SARS-CoV-2 S protein of each strain/variant in a combination listed in Table D is administered to a subject previously administered (i) one or more doses of a composition that delivers a SARS-CoV-2 S protein of a Wuhan strain and (ii) one or more doses of a bivalent composition (e.g., an RNA composition) that delivers a SARS-CoV-2 S protein of a Wuhan strain and a SARS-CoV-2 S protein of an Omicron BA.4/5 variant. In some embodiments, an RNA composition that comprises RNA(s) encoding a SARS-CoV-2 S protein of a strain/variant listed in Table D, or a SARS-CoV-2 S protein of each strain/variant in a combination listed in Table D is administered as a booster dose to a subject previously administered a priming dosing regimen of a composition (e.g., an RNA composition) that delivers a SARS-CoV-2 S protein of a Wuhan strain and at least one booster dose of a bivalent composition that delivers a SARS-CoV-2 S protein of a Wuhan strain and a SARS-CoV-2 S protein of an Omicron BA.4/5 variant.

In some embodiments, an RNA composition that comprises RNA(s) encoding a SARS-CoV-2 S protein of a strain/variant listed in Table D, or a SARS-CoV-2 S protein of each strain/variant in a combination listed in Table D is administered as a booster dose to a subject previously administered:
A first dose of an RNA vaccine that delivers a SARS-CoV-2 S protein of a Wuhan strain, and a second dose of a bivalent composition comprising a first RNA encoding a SARS-CoV-2 S protein of a Wuhan strain and a second RNA encoding a SARS-CoV-2 S protein of an Omicron BA.4/5 variant, where the bivalent composition is administered about 21 days after the most recent dose of a composition that delivers a SARS-CoV-2 S protein of a Wuhan strain;
two doses (administered about 21 days apart) of an RNA vaccine that delivers a SARS-CoV-2 S protein of a Wuhan strain, and a dose of a bivalent composition comprising a first RNA encoding a SARS-CoV-2 S protein of a Wuhan strain and a second RNA encoding a SARS-CoV-2 S protein of an Omicron BA.4/5 variant, where the bivalent composition is administered at least about two months after the most recent dose of a composition that delivers a SARS-CoV-2 S protein of a Wuhan strain;
three doses of an RNA vaccine that delivers a SARS-CoV-2 S protein of a Wuhan strain (where the first and the second dose are administered about 21 days apart and the third dose is administered about 28 days after the second dose) and at least one dose of a bivalent vaccine comprising a first RNA encoding a SARS-CoV-2 S protein of a Wuhan strain and a second RNA encoding a SARS-CoV-2 S protein of an Omicron BA.4/5 variant, where the bivalent composition is administered at least about 2 months after the most recent dose of a composition that delivers a SARS-CoV-2 S protein of a Wuhan strain;
three doses of an RNA vaccine that delivers a SARS-CoV-2 S protein of a Wuhan strain (where the first and the second dose are administered about 21 days apart and the third dose is administered at least about 2 months after the second dose) and at least one dose of a bivalent vaccine comprising a first RNA encoding a SARS-CoV-2 S protein of a Wuhan strain and a second RNA encoding a SARS-CoV-2 S protein of an Omicron BA.4/5 variant, where the bivalent composition is administered at least about 2 months after the most recent dose of a composition that delivers a SARS-CoV-2 S protein of a Wuhan strain.
four doses of an RNA vaccine that delivers a SARS-CoV-2 S protein of a Wuhan strain (where the first and the second dose are administered about 21 days apart, the third dose is administered about 28 days after the second dose, and the fourth dose is administered at least about 2 months after the third dose) and at least one dose of a bivalent vaccine comprising a first RNA encoding a SARS-CoV-2 S protein of a Wuhan strain and a second RNA encoding a SARS-CoV-2 S protein of an Omicron BA.4/5 variant, where the bivalent composition is administered at least about 2 months after the most recent dose of a composition that delivers a SARS-CoV-2 S protein of a Wuhan strain; or
four doses of an RNA vaccine that delivers a SARS-CoV-2 S protein of a Wuhan strain (where the first and the second dose are administered about 21 days apart, the third dose is administered at least about 2 months after the second dose, and the fourth dose is administered at least about 4 months after the third dose) and at least one dose of a bivalent vaccine comprising a first RNA encoding a SARS-CoV-2 S protein of a Wuhan strain and a second RNA encoding a SARS-CoV-2 S protein of an Omicron BA.4/5 variant, where the bivalent composition is administered at least about 2 months after the most recent dose of a composition that delivers a SARS-CoV-2 S protein of a Wuhan strain

In some embodiments, a subject is administered a dosing regimen comprising doses of both a SARS-CoV-2 vaccine and a SARS-CoV-2/influenza combination vaccine (e.g., a dosing regimen listed in the below Table E).

TABLE E
Exemplary Dosing Regimens comprising 3 doses

| | | 3^{rd} dose (SARS-CoV-2/ |
|---|---|---|
| | | Influenza combination vaccine, |
| 1^{st} Dose | 2^{nd} Dose | delivering SARS-CoV-2 |
| (SARS-CoV-2 | (SARS-CoV-2 | antigens from the below- |
| vaccine) | vaccine) | listed variants/strains) |
| BNT162b2 | BNT162b2 + BA.4/5 (bivalent) | BNT162b2 + BA.4/5 |
| BNT162b2 | BNT162b2 + BA.4/5 (bivalent) | XBB.1.5 |
| BNT162b2 | BNT162b2 + BA.4/5 (bivalent) | BA.2.75.2 |
| BNT162b2 | BNT162b2 + BA.4/5 (bivalent) | BQ.1.1 |
| BNT162b2 | BNT162b2 + BA.4/5 (bivalent) | BNT162b2 + BA.4/5 + XBB.1.5 |
| BNT162b2 | BNT162b2 + BA.4/5 (bivalent) | BNT162b2 + BA.4/5 + BA.2.75.2 |
| BNT162b2 | BNT162b2 + BA.4/5 (bivalent) | BA.4/5 + XBB.1.5 |
| BNT162b2 | BNT162b2 + BA.4/5 (bivalent) | BA.4/5 + BA.2.75.2 |
| BNT162b2 | BNT162b2 + BA.4/5 (bivalent) | BA.4/5 + BQ.1.1 |

In some embodiments, a subject is administered a dosing regimen comprising doses of both a SARS-CoV-2 vaccine and a SARS-CoV-2/influenza combination vaccine (e.g., a dosing regimen listed in the below Table F).

TABLE F
Exemplary Dosing Regimens comprising 4 doses

| | | | 4^{th} dose (SARS-CoV-2/ |
|---|---|---|---|
| | | | Influenza combination vaccine, |
| 1^{st} Dose | 2^{nd} Dose | 3^{rd} Dose | delivering SARS-CoV-2 |
| (SARS-CoV-2 | (SARS-CoV-2 | (SARS-CoV-2 | antigens from the below- |
| vaccine) | vaccine) | vaccine) | listed variants/strains) |
| BNT162b2 | BNT162b2 | BNT162b2 + BA.4/5 (bivalent) | XBB.1.5 |
| BNT162b2 | BNT162b2 | BNT162b2 + BA.4/5 (bivalent) | BA.2.75.2 |
| BNT162b2 | BNT162b2 | BNT162b2 + BA.4/5 (bivalent) | BQ.1.1 |
| BNT162b2 | BNT162b2 | BNT162b2 + BA.4/5 (bivalent) | BA.4/5 + XBB.1.5 |
| BNT162b2 | BNT162b2 | BNT162b2 + BA.4/5 (bivalent) | BA.4/5 + BA.2.75.2 |
| BNT162b2 | BNT162b2 | BNT162b2 + BA.4/5 (bivalent) | BA.4/5 + BQ.1.1 |

In some embodiments, a dosing regimen listed in Table E or F is administered to a vaccine naïve subject. In some embodiments, a first dose and a second dose listed in Table E or F are administered about 21 days apart. In some embodiments, a third dose listed in Table E or F is administered at least about 2 months after a second dose. In some embodiments, a fourth dose listed in Table F is administered at least about two months after a third dose.

In some embodiments, at least one single dose is administered. In some embodiments, such single dose is administered to subjects, for example, who may have previously received one or more doses of, or a complete regimen of, a SARS-CoV-2 vaccine (e.g., of a BNT162b2 vaccine [including, e.g., as described herein], an mRNA-1273 vaccine, an Ad26.CoV2.S vaccine, a ChAdxOx1 vaccine, an NVX-CoV2373 vaccine, a CvnCoV vaccine, a GAM-COVID0Vac vaccine, a CoronaVac vaccine, a BBIBP-CorV vaccine, an Ad5-nCOV vaccine, a zf2001 vaccine, a SCB-2019 vaccine, a JNJ 78436735 vaccine, or other approved mRNA or adenovector vaccines, etc. In some embodiments, at least one single dose is administered to subjects who may have previously received one or more doses of, or a complete regimen of a SARS-CoV-2 RNA vaccine, e.g., as described in WO2021/154763, WO2021156267A1, or WO2021239880A1. In some embodiments, at least one single dose is administered to a subject who has previously been administered at least three doses of US-authorized COVID-19 vaccine(s) (e.g., an mRNA vaccine, e.g., wherein the most recent dose has been a variant adapted booster shot). In some embodiments, at least one single dose is administered to a subject who has previously received one or more doses of, or a complete regimen of, an influenza vaccine (e.g., Fluzone®, Fluzone High-Dose®, Fluarix®, FluAd®, Flucelvax®, FluBlok Quadrivalent®, FluMist Quadrivalent®, or other approved vaccines). In some embodiments, at least one single dose is administered to a subject who has previously received one or more doses of, or a complete regimen of, an RNA influenza vaccine, e.g., as described in WO2017070620A2 or WO2021239880A1. Alternatively or additionally, in some embodiments, a single dose is administered to subjects who have been exposed to and/or infected by SARS-CoV-2 and/or influenza. In some embodiments, at least one single dose is administered to subjects who both have received one or more doses of, or a complete regimen of, a SARS-CoV-2 vaccine and an influenza vaccine and have been exposed to and/or infected with SARS-CoV-2 and/or influenza.

In some particular embodiments where at least one single dose is administered to subjects who have received one or more doses of a prior SARS-CoV-2 vaccine, such prior SARS-CoV-2 vaccine is a different vaccine, or a different form (e.g., formulation) and/or dose of a vaccine with the same active (e.g., BNT162b2); in some such embodiments, such subjects have not received a complete regimen of such prior vaccine and/or have experienced one or more undesirable reactions to or effects of one or more received doses of such prior vaccine. In some particular embodiments, such prior vaccine is or comprises higher dose(s) of the same active (e.g., BNT162b2). Alternatively or additionally, in some such embodiments, such subjects were exposed to and/or infected by SARS-CoV-2 prior to completion (but, in some embodiments, after initiation) of a full regimen of such prior vaccine.

In one embodiment, at least two doses are administered. For example, a second dose may be administered about 21 days following administration of the first dose.

In some embodiments, an mRNA composition described herein is administered to a subject who has previously been administered at least two doses of BNT162b2 (e.g., two doses of BNT162b2 administered about 21 days apart).

In some embodiment, an RNA composition described herein is administered to a subject who has previously been administered a vaccine that delivers an antigen of a SARS-CoV-2 variant (e.g., an Omicron BA.4/5 variant (e.g., a vaccine described herein)).

In one embodiment, at least three doses are administered. In some embodiments, such third dose is administered a period of time after the second dose that is comparable to (e.g., the same as) the period of time between the first and second doses. For example, in some embodiments, a third dose may be administered about 21 days following administration of the second dose. In some embodiments, a third dose is administered after a longer period of time relative to the second dose than the second dose was relative to the first dose. In some embodiments, a three-dose regimen is administered to an immunocompromised patient, e.g., a cancer patient, an HIV patient, a patient who has received and/or is receiving immunosuppressant therapy (e.g., an organ transplant patient). In some embodiments, the length of time between the second and third dose (e.g., a second and third dose administered to an immunocompromised patient) is at least about 21 days (e.g., at least about 28 days). In some embodiments, a vaccination regimen comprises administering the same amount of RNA in different doses (e.g., in first and/or second and/or third and/or subsequent doses). In some embodiments, a vaccination regimen comprises administering different amounts of RNA in different doses. In some embodiments, one or more later doses is larger than one or more earlier doses (e.g., in situations where waning of vaccine efficacy from one or more earlier doses is observed and/or immune escape by a variant (e.g., one described herein) that is prevalent or rapidly spreading is observed in a relevant jurisdiction at the time of administration is observed). In some embodiments, one or more later doses may be larger than one or more earlier doses by at least 30%, at least 40%, at least 50%, at least 60%, at least 70%, at least 80%, at least 90%, at least 95%, or higher, provided that safety and/or tolerability of such a dose is clinically acceptable. In some embodiments, one or more later doses may be larger than one or more earlier doses by at least 1.1-fold, at least 1.5-fold, at least 2-fold, at least 3-fold, at least 4-fold, or higher provided that safety and/or tolerability of such a dose is clinically acceptable. In some embodiments, one or more later doses is smaller than one or more earlier doses (e.g., in a negative reaction was experienced after one or more earlier doses and/or if exposure to and/or infection by SARS-CoV-2 between an earlier dose and a subsequent dose). In some embodiments, one or more later doses may be smaller than one or more earlier doses by at least 10%, at least 20%, at least 30%, at least 40%, at least 50%, at least 60%, or higher. In some embodiments, where different doses are utilized, they are related to one another by identity with and/or dilution of a common stock as described herein.

In some embodiments, where at least two or more doses are administered (e.g., at least two doses administered in a primary regimen, at least two doses administered in a booster regimen, or at least one dose administered in a primary regimen and at least one dose in a booster regimen), the same RNA compositions described herein may be administered in such doses and each of such doses can be the same or different (as described herein). In some embodiments, where at least two or more doses are administered (e.g., at least two doses administered in a primary regimen, at least two doses administered in a booster regimen, or at least one dose administered in a primary regimen and at least one dose in a booster regimen), different RNA compositions described herein (e.g., different encoded viral polypeptides, e.g., from different coronavirus clades, or from different strains of the same coronavirus clade; different construct elements such as 5′ cap, 3′ UTR, 5′ UTR, etc.; different formulations, e.g., different excipients and/or buffers (e.g., PBS vs. Tris); different LNP compositions; or combinations thereof) may be administered in such doses and each of such doses can be the same or different (e.g., as described herein).

In some embodiments, a subject is administered two or more RNAs (e.g., as part of either a primary regimen or a booster regimen), wherein the two or more RNAs are administered on the same day or same visit. In some embodiments, the two or more RNAs are administered in separate compositions, e.g., by administering each RNA to a separate part of the subject (e.g., by intramuscular administration to different arms of the subject or to different sites of the same arm of the subject). In some embodiments, the two or more RNAs are mixed prior to administration (e.g., mixed immediately prior to administration, e.g., by the administering practitioner). In some embodiments, the two or more RNAs are formulated together (e.g., by (a) mixing separate populations of LNPs, each population comprising a different RNA; or (b) by mixing two or more RNAs prior to LNP formulation, so that each LNP comprises two or more RNAs). In some embodiments, the two or more RNAs comprise an RNA that encode a coronavirus S protein or immunogenic fragment thereof (e.g., RBD or other relevant domains) from one strain (e.g., Wuhan strain) and a variant that is prevalent or rapidly spreading in a relevant jurisdiction at the time of administration (e.g., a variant described herein). In some embodiments, such a variant is an Omicron variant (e.g., a BA.1, BA.2, BA.3, BA.4/5, or XBB.1.5 variant). In some embodiments, the two or more RNAs comprise a first RNA and a second RNA that have been shown to elicit a broad immune response in subject. In some embodiments the two or more RNAs comprise an RNA encoding a SARS-CoV-2 S protein from a Wuhan strain and an RNA encoding a SARS-CoV-2 S protein from a BA.1 Omicron variant. In some embodiments the two or more RNAs comprise an RNA encoding a SARS-CoV-2 S protein from a Wuhan strain and an RNA encoding a SARS-CoV-2 S protein from a BA.2 Omicron variant. In some embodiments the two or more RNAs comprise an RNA encoding a SARS-CoV-2 S protein from a Wuhan strain and an RNA encoding a SARS-CoV-2 S protein from a BA.4 or BA.5 Omicron variant. In some embodiments the two or more RNAs comprise an RNA encoding a SARS-CoV-2 S protein from a BA.1 Omicron variant and an RNA encoding a SARS-CoV-2 S protein from a BA.2 Omicron variant. In some embodiments the two or more RNAs comprise an RNA encoding a SARS-CoV-2 S protein from a BA.1 Omicron variant and an RNA encoding a SARS-CoV-2 S protein from a BA.4 or BA.5 Omicron variant. In some embodiments the two or more RNAs comprise an RNA encoding a SARS-CoV-2 S protein from a BA.2 Omicron variant and an RNA encoding a SARS-CoV-2 S protein from a BA.4 or 5 Omicron variant. In some embodiments the two or more RNAs comprise an RNA encoding a SARS-CoV-2 S protein from a Wuhan strain, an alpha variant, a beta variant, or a delta variant, or sublineages derived therefrom; and an RNA encoding a SARS-CoV-2 S protein from a BA.2, BA.4 or 5 Omicron variant, or sublineages derived therefrom.

In some embodiments, a subject may be administered any one of combinations 1 to 66, listed in the below table. In some embodiments, such combinations can be administered using an LNP formulation, where the first RNA and the second RNA are encapsulated in the same LNP or in separate LNPs. In some embodiments, such combinations can be administered as separate LNP formulations (e.g., by administering at separate sites to a subject).

| | SARS-CoV-2 S protein | SARS-CoV-2 S protein |
|---|---|---|
| Combination | encoded by a first RNA¹ | encoded by a second RNA¹ |
| 1 | Wuhan | Alpha |
| 2 | Wuhan | Beta |
| 3 | Wuhan | Delta |
| 4 | Wuhan | BA.1 |
| 5 | Wuhan | BA.2 |
| 6 | Wuhan | BA.2.12.1 |
| 7 | Wuhan | BA.3 |
| 8 | Wuhan | BA.4/5 |
| 9 | Wuhan | XBB |
| 10 | Wuhan | XBB variant (e.g., |
| | | XBB.1, XBB.2, XBB.1.3) |
| 11 | Wuhan | BQ.1.1 |
| 12 | Alpha | Beta |
| 13 | Alpha | Delta |
| 14 | Alpha | BA.1 |
| 15 | Alpha | BA.2 |
| 16 | Alpha | BA.2.12.1 |
| 17 | Alpha | BA.3 |
| 18 | Alpha | BA.4/5 |
| 19 | Alpha | XBB |
| 20 | Alpha | XBB variant (e.g., |
| | | XBB.1, XBB.2, XBB.1.3) |
| 21 | Alpha | BQ.1.1 |
| 22 | Beta | Delta |
| 23 | Beta | BA.1 |
| 24 | Beta | BA.2 |
| 25 | Beta | BA.2.12.1 |
| 26 | Beta | BA.3 |
| 27 | Beta | BA.4/5 |
| 28 | Beta | XBB |
| 29 | Beta | XBB variant (e.g., |
| | | XBB.1, XBB.2, XBB.1.3) |
| 30 | Beta | BQ.1.1 |
| 31 | Delta | BA.1 |
| 32 | Delta | BA.2 |
| 33 | Delta | BA.2.12.1 |
| 34 | Delta | BA.3 |
| 35 | Delta | BA.4/5 |
| 36 | Delta | XBB |
| 37 | Delta | XBB variant (e.g., |
| | | XBB.1, XBB.2, XBB.1.3) |
| 38 | Delta | BQ.1.1 |
| 39 | BA.1 | BA.2 |
| 40 | BA.1 | BA.2.12.1 |
| 41 | BA.1 | BA.3 |
| 42 | BA.1 | BA.4/5 |
| 43 | BA.1 | XBB |
| 44 | BA.1 | XBB variant (e.g., |
| | | XBB.1, XBB.2, XBB.1.3) |
| 45 | BA.1 | BQ.1.1 |
| 46 | BA.2 | BA.2.12.1 |
| 47 | BA.2 | BA.3 |
| 48 | BA.2 | BA.4/5 |
| 49 | BA.2 | XBB |
| 50 | BA.2 | XBB variant (e.g., |
| | | XBB.1, XBB.2, XBB.1.3) |
| 51 | BA.2 | BQ.1.1 |
| 52 | BA.2.12.1 | BA.3 |
| 53 | BA.2.12.1 | BA.4/5 |
| 54 | BA.2.12.1 | XBB |
| 55 | BA.2.12.1 | XBB variant (e.g., |
| | | XBB.1, XBB.2, XBB.1.3) |
| 56 | BA.2.12.1 | BQ.1.1 |
| 57 | BA.3 | BA.4/5 |
| 58 | BA.3 | XBB |
| 59 | BA.3 | XBB variant (e.g., |
| | | XBB.1, XBB.2, XBB.3) |
| 60 | BA.3 | BQ.1.1 |
| 61 | BA.4/5 | XBB |
| 62 | BA.4/5 | XBB variant (e.g., |
| | | XBB.1, XBB.2, XBB.1.3) |
| 63 | BA.4/5 | BQ.1.1 |
| 64 | XBB | XBB variant (e.g., |
| | | XBB.1, XBB.2, XBB.1.3) |
| 65 | XBB | BQ.1.1 |
| 66 | XBB variant (e.g., | BQ.1.1 |
| | XBB.1, XBB.2, XBB.1.3) | |

¹Listed RNAs encode a SARS-CoV-2 S protein having mutations characteristic of the indicated SARS-CoV-2 variant.

In some embodiments, a subject is administered or a composition comprises one or more first RNAs and one or more second RNAs, each in the same amount (i.e., at a 1:1 ratio).

In some embodiments, a subject is administered or a composition comprises one or more first RNAs and one or more second RNAs, each in a different amount. For example, in some embodiments, a subject is administered or a composition comprises one or more first RNAs in an amount that is 0.01 to 100 times that of one or more second RNAs (e.g., wherein the amount of the one or more first RNAs is 0.01 to 50, 0.01 to 4, 0.01 to 30, 0.01 to 25, 0.01 to 20, 0.01 to 15, 0.01 to 10, 0.01 to 9, 0.01 to 8, 0.01 to 7, 0.01 to 6, 0.01 to 5, 0.01 to 4, 0.01 to 3, 0.01 to 2, 0.01 to 1.5, 1 to 50, 1 to 4, 1 to 30, 1 to 25, 1 to 20, 1 to 15, 1 to 10, 1 to 9, 1 to 8, 1 to 7, 1 to 6, 1 to 5, 1 to 4, 1 to 3, 1 to 2, or 1 to 1.5 times that of the one or more second RNAs). In some embodiments, a subject is administered or a composition comprises one or more first RNAs and one or more second RNAs, wherein the concentration of the one or more first RNAs is 1 to 10 times that of the one or more second RNAs. In some embodiments, a subject is administered or a composition comprises one or more first RNAs and one or more second RNAs, wherein the amount of the one or more first RNAs is 1 to 5 times that of the one or more second RNAs. In some embodiments, a subject is administered or a composition comprises one or more first RNAs and one or more second RNAs, wherein the concentration of the one or more first RNAs is 1 to 3 times that of the one or more second RNAs. In some embodiments, a subject is administered or a composition comprises one or more first RNAs and one or more second RNAs, wherein the amount of the one or more first RNAs is 2 times that of the one or more second RNAs. In some embodiments, a subject is administered or a composition comprises one or more first RNAs and one or more second RNAs, wherein the concentration of the one or more first RNAs is 3 times that of the one or more second RNAs.

In some embodiments, a subject is administered or a composition comprises two first RNAs, each encoding a SARS-CoV-2 S protein of a different SARS-CoV-2 strain or variant, and each in the same amount (i.e., at a 1:1 ratio).

In some embodiments, a subject is administered or a composition comprises two first RNAs, each encoding an S protein of a different SARS-CoV-2 strain or variant, wherein the amount of each RNA is not the same. For example, in some embodiments, the ratio of the two first RNAs is 1:0.01-100 (e.g., 1:0.01-50; 1:0.01-40; 1:0.01-30; 1:0.01-25; 1:0.01-20; 1:0.01-15; 1:0.01-10; 1:0.01-9; 1:0.01-8; 1:0.01-7; 1:0.01-6; 1:0.01-5; 1:0.01-4; 1:0.01-3; 1:0.01-2; 1:0.01-1.5, 1:0.1-10, 1:0.1-5, 1:0.1-3, 1:2-10, 1:2-5, or 1:2-3). In some embodiments, a subject is administered or a composition comprises two first RNAs at a ratio of 1:3. In some embodiments, a subject is administered or a composition comprises two first RNAs at a ratio of 1:2.

In some embodiments, a subject is administered or a composition comprises three first RNAs, each encoding a SARS-CoV-2 S protein of a different SARS-CoV-2 strain or variant, and each in the same amount (i.e., at a 1:1:1 ratio).

In some embodiments, a subject is administered or a composition comprises three first RNAs, each encoding an S protein of a different SARS-CoV-2 strain or variant, wherein the amount of each RNA is not the same (e.g., one RNA is present in an amount that is different than the other two RNA, or all three RNAs are present in different amounts). For example, in some embodiments, the ratio of the three first RNAs is 1:0.01-100:0.01-100 (e.g., 1:0.01-50:0.01-50; 1:0.01-40:0.01-40; 1:0.01-30:0.01-30; 1:0.01-25:0.01-25; 1:0.01-20:0.01-20; 1:0.01-15:0.01-15; 1:0.01-10:0.01-10; 1:0.01-9:0.01-9; 1:0.01-8:0.01-8; 1:0.01-7:0.01-7; 1:0.01-6:0.01-6; 1:0.01-5:0.01-5; 1:0.01-4:0.01-4; 1:0.01-3:0.01-3; 1:0.01-2:0.01-2; 1:0.01-1.5:0.01-1.5; 1:0.1-10:0.1-10, 1:0.1-5:0.1-5, 1:0.1-3:0.1-3, 1:2-10:2-10, 1:2-5:2-5, or 1:2-3:2-3). In some embodiments, a subject is administered or a composition comprises three first RNAs at a ratio of 1:1:3. In some embodiments, a subject is administered or a composition comprises three first RNAs at a ratio of 1:3:3.

In some embodiments, a subject is administered or a composition comprises two or more second RNAs, one or more of which encode an HA protein of a Type A influenza virus, and one or more of which encode an HA protein of a Type B influenza virus. In some embodiments, the one or more second RNAs that encode an HA protein of a Type A influenza virus and the one or more second RNAs that encode an HA protein of a Type B influenza virus are present or are administered in the same amount (i.e., at a ratio of 1:1). In some embodiments, the one more second RNAs that encode an HA protein of a Type A influenza virus and the one or more second RNAs that encode an HA protein of a Type B influenza virus are administered in different amounts (e.g., in a ratio of between 1:10 and 10:1, or in a ratio of 1:2, 1:3, 1:4, 1:5, 2:1, 3:1, 4:1, or 5:1 (total RNA encoding an A antigen: total RNA encoding a B antigen).

In some embodiments, a subject is administered or a composition comprises two second RNAs, each encoding an HA protein of a different influenza virus type (e.g., a second RNA encoding an HA protein of a Type A influenza virus and a second RNA encoding an HA protein of a Type B influenza virus). In some embodiments, the second RNAs are administered or are present in the same amount (i.e., at a 1:1 ratio). In some embodiments, the second RNAs are administered or are present in different amounts (e.g., in a ratio of between 1:10 and 10:1, or in a ratio of 1:2, 1:3, 1:4, 1:5, 2:1, 3:1, 4:1, or 5:1 (A: B)).

In some embodiments, a subject is administered or a composition comprises three second RNAs, each encoding an HA protein of a different influenza virus subtype (e.g., an HA protein of an A/Wisconsin (H1N1) virus, an A/Darwin (H3N2) virus, and a B/Austria (Victoria) virus). In some embodiments, a subject is administered or a composition comprises each of the three second RNAs in the same amount (i.e., at a 1:1:1 ratio). In some embodiments, a subject is administered or a composition comprises a different amount of one or more of the three second RNAs (e.g., in a ratio of between 1:1:2 and 1:1:10 (e.g., in a ratio of 1:1:2, 1:1:3, 1:1:4, or 1:1:5), or in a ratio of between 2:2:1 and 2:2:10, (e.g., in a ratio of 2:2:1, 3:3:1, 4:4:1, or 5:5:1). In some embodiments, a subject is administered or a composition comprises three second RNAs, two of which encode HA proteins of different influenza type A virus, and one of which encodes an HA protein of an influenza type B virus. In some such embodiments, the second RNA encoding an HA protein of an influenza type B virus is present or is administered in a higher amount as compared to either second RNA encoding an HA protein from a type A virus (e.g., in some embodiments, the ratios of the two second RNAs encoding HA proteins from type A influenza viruses relative to the second RNA encoding an HA protein from a type B influenza virus is 1:1:1-10, 1:1:2, 1:1:3, 1:1:4, or 1:1:5 (A:A:B)). In some embodiments, a subject is administered or a composition comprises three second RNAs, two encoding an HA protein of an influenza type A virus and one encoding an HA protein of an influenza type B virus, wherein the ratio of the three second RNAs 1:1:4 (A:A:B). In some embodiments, the two second RNAs encoding an HA protein of an influenza type A viruses are each present or are each administered in a higher amount as compared to the second RNA encoding an HA protein from a type B virus (e.g., in some embodiments, the ratios of the two second RNAs encoding HA proteins from type A influenza viruses relative to the second RNA encoding an HA protein from a type B influenza virus is 1-10:1-10:1, 2:2:1, 3:3:1, 4:4:1, or 5:5:1 (A:A:B)).

In some embodiments, a subject is administered or a composition comprises four second RNAs, each encoding an HA protein of a different influenza virus subtype. In some such embodiments, the four second RNAs comprise two second RNAs encoding HA proteins of different influenza type A viruses and two second RNAs encoding HA proteins of different influenza type B virus (e.g., an HA protein of an H1N1 virus, an HA protein of an H3N2 virus, an HA protein of a B/Victoria lineage virus, and an HA protein of a B/Yamagata lineage virus). In some embodiments, each of the two second RNAs encoding an HA protein of an influenza type A virus and each of the two second RNAs encoding an HA protein of an influenza type B virus are present in the same amount (i.e., the ratio of the four second RNAs is 1:1:1:1). In some embodiments, the two second RNAs encoding an HA protein of an influenza type B virus are each administered or are each present in a higher amount as compared to either second RNA encoding an HA protein from a type A virus (e.g., in some embodiments, the ratios of the two second RNAs encoding HA proteins from type A influenza viruses relative to the two second RNAs encoding an HA protein from a type B influenza virus is 1:1:2-10:2-10, 1:1:2-5:2-5, 1:1:2:2, 1:1:3:3, 1:1:4:4, 1:1:5:5, 1:1:6:6, 1:1:7:7, 1:1:8:8, 1:1:9:9, 1:1:10:10 (A:A:B: B)). In some embodiments, a subject is administered or a composition comprises four second RNAs, two encoding an HA protein of an influenza type A virus and two encoding an HA protein of an influenza type B virus, wherein the ratio of the four second RNAs 1:1:5:5 (A:A:B: B). In some embodiments, the two second RNAs encoding an HA protein of an influenza type A virus are each administered or are each present in a higher amount as compared to either second RNA encoding an HA protein from a type B virus (e.g., in some embodiments, the ratios of the two second RNAs encoding HA proteins from type A influenza viruses relative to the two second RNAs encoding an HA protein from a type B influenza virus is 2-10:2-10:1:1, 2-5:2-5:1:1, 2:2:1:1, 3:3:1:1, 4:4:1:1, 5:5:1:1, 6:6:1:1, 7:7:1:1, 8:8:1:1, 9:9:1:1, 10:10:1:1 (A:A:B: B)).

In some embodiments, a composition comprises or a subject is administered four second RNAs, comprising three second RNAs that encode HA proteins of different influenza type A viruses and one second RNA encoding an HA protein of an influenza type B virus (e.g., A/Wisconsin (H1N1), A/Darwin (H3N2), A/Cambodia (H3N2), and B/Austria (Victoria)). In some such embodiments, each of the four second RNAs is administered or is present in the same amount (i.e., at a 1:1:1:1 ratio). In some embodiments, the amount of second RNA encoding an HA protein of an influenza type B virus is higher than any one of the second RNAs encoding an HA protein of an influenza type A virus (e.g., in some embodiments, the ratio of second RNAs is 1:1:1:1-10, 1:1:1:1-5,1:1:1:2, 1:1:1:3, 1:1:1:4, or 1:1:1:5 (A:A:A:B)). In some embodiments, the ratio of second RNAs administered or in a composition is 1:1:1:5 (A:A:A:B). In some embodiments, the amount of each of the second RNAs encoding an HA protein of an influenza type A virus is higher than that of the second RNA encoding an HA protein of an influenza type B virus (e.g., in some embodiments, the ratio of second RNAs is 1-10:1-10:1-10:1, 1-5:1-5:1-5:1, 2:2:2:1, 3:3:3:1, 4:4:4:1, or 5:5:5:1 (A:A:A:B)).

In some embodiments, a subject is administered or a composition comprises one or more second RNAs encoding an HA protein of an influenza virus (e.g., two second RNAs, three second RNAs, or four second RNAs, each encoding an HA protein of a different influenza virus) in a total amount of 0.1 to 100 μg (e.g., 1 to 90 μg, 3 to 90 μg, 1 to 60 μg, 3 to 60 μg, 5 to 60 μg, 10 to 60 μg, 30 to 60 μg, 3 to 30 μg). In some embodiments, a subject is administered or a composition comprises one or more second RNAs encoding an HA protein of an influenza virus in a total amount of 3 μg, 5 μg, 6 μg, 10 μg, 15 μg, 20 μg, 25 μg, 30 μg, 45 μg, 60 μg, 75 μg, or 90 μg.

In some embodiments, a subject is administered or a composition comprises three or four second RNAs, each encoding an HA antigen of a different influenza strain, in one of the amounts listed in the below Table C (each “Influenza Component” corresponding to a second RNA encoding an HA antigen (e.g., a second RNA as described herein).

TABLE C
Exemplary Amounts of Second RNAs Encoding HA Antigens

| Combination | Influenza | | Influenza | | Influenza | | Influenza | | |
|---|---|---|---|---|---|---|---|---|---|
| # | Component 1 | | Component 2 | | Component 3 | | Component 4 | | Total |
| 1 | 7.5 | μg (A type) | 7.5 | μg (A type) | 7.5 | μg (B type) | 7.5 | μg (B type) | 30 μg |
| 2 | 15 | μg (A type) | 15 | μg (A type) | 15 | μg (B type) | 15 | μg (B type) | 60 μg |
| 3 | 11.25 | μg (A type) | 11.25 | μg (A type) | 11.25 | μg (B type) | 11.25 | μg (B type) | 45 μg |
| 4 | 5 | μg (A type) | 5 | μg (A type) | 25 | μg (B type) | 25 | μg (B type) | 60 μg |
| 5 | 2.5 | μg (A type) | 2.5 | μg (A type) | 12.5 | μg (B type) | 12.5 | μg (B type) | 30 μg |
| 6 | 7.5 | μg (A type) | 7.5 | μg (A type) | 30 | μg (B type) | — | | 45 μg |
| 7 | 7.5 | μg (A type) | 7.5 | μg (A type) | 7.5 | μg (A type) | 7.5 | μg (B type) | 30 μg |

In some embodiments, a subject is administered or a composition comprises three or four RNAs encoding an influenza HA protein in one of the amounts listed in Table C, and one or more RNAs encoding a SARS-CoV-2 S protein. In some embodiments, a subject is administered or a composition comprises three or four RNAs encoding an influenza HA protein in one of the amounts listed in Table C, and two or more RNAs encoding a SARS-CoV-2 antigen (e.g., two or more RNAs, each encoding an S protein of a different SARS-CoV-2 strain or variant). In some embodiments, a combination vaccine comprises three or four RNAs in one of the amounts listed in Table C, and 30 μg of a bivalent vaccine (e.g., a bivalent vaccine comprising: (i) 15 μg of an RNA encoding an S protein of a first SARS-CoV-2 strain or variant and 15 μg of a second strain or variant (e.g., wherein the first and second strains or variants are recommended for inclusion in an updated vaccine by an appropriate health agency); (ii) 15 μg of an RNA encoding a SARS-CoV-2 S protein of an Omicron BA.4/5 variant and 15 μg of an RNA encoding an S protein of a second SARS-CoV-2 strain or variant; or (iii) 15 μg of an RNA encoding a SARS-CoV-2 S protein of an Omicron BA.4/5 variant and 15 μg of an RNA encoding an S protein of a SARS-CoV-2 Wuhan strain). In some embodiments, a combination vaccine comprises three or four RNAs in one of the amounts listed in Table C, and 60 μg of a bivalent vaccine (e.g., a bivalent vaccine comprising: (i) 30 μg of an RNA encoding an S protein of a first SARS-CoV-2 strain or variant and 30 μg of a second strain or variant (e.g., wherein the first and second strains or variants are recommended for inclusion in an updated vaccine by an appropriate health agency); (ii) 30 μg of an RNA encoding a SARS-CoV-2 S protein of an Omicron BA.4/5 variant and 30 μg of an RNA encoding an S protein of a second SARS-CoV-2 strain or variant; or (iii) 30 μg of an RNA encoding a SARS-CoV-2 S protein of an Omicron BA.4/5 variant and 30 μg of an RNA encoding an S protein of a SARS-CoV-2 Wuhan strain).

In some embodiments, a vaccination regimen comprises a first vaccination regimen (e.g., a primary regimen) that includes at least two doses of an RNA composition as described herein, e.g., wherein the second dose may be administered about 21 days following administration of the first dose, and a second vaccination (e.g., a booster regimen) that comprises a single dose or multiple doses, e.g., two doses, of an RNA composition as described herein. In some embodiments, doses of a booster regimen are related to those of a primary regimen by identity with or dilution from a common stock as described herein. In various embodiments, a booster regimen is administered (e.g., is initiated) at least 1 week, at least 2 weeks, at least 3 weeks, at least 1 month, at least 2 months, at least 3 months, at least 4 months, at least 5 months, or at least 6 months, at least 7 months, at least 8 months, at least 9 months, at least 10 months, at least 11 months, at least 12 months, or longer, after administration of a primary regimen, e.g., after completion of a primary regimen comprising at least two doses. In various embodiments, a booster regimen is administered (e.g., is initiated) 1-12 months, 2-12 months, 3-12 months, 4-12 months, 6-12 months, 1-6 months, 1-5 months, 1-4 months, 1-3 months, or 2-3 months after administration of a primary regimen, e.g., after completion of a primary regimen comprising at least two doses. In various embodiments, a booster regimen is administered (e.g., is initiated) 1 to 60 months, 2 to 48 months, 2 to 24 months, 3 to 24 months, 6 to 18 months, 6 to 12 months, or 5 to 7 months after administration of a primary regimen, e.g., after completion of a two-dose primary regimen. In some embodiments, each dose of a primary regimen is about 60 μg per dose. In some embodiments, each dose of a primary regimen is about 50 μg per dose. In some embodiments, each dose of a primary regimen is about 30 μg per dose. In some embodiments, each dose of a primary regimen is about 25 μg per dose. In some embodiments, each dose of a primary regimen is about 20 μg per dose. In some embodiments, each dose of a primary regimen is about 15 μg per dose. In some embodiments, each dose of a primary regimen is about 10 μg per dose. In some embodiments, each dose of a primary regimen is about 3 μg per dose. In some embodiments, each dose of a booster regimen is the same as that of the primary regimen. In some embodiments, each dose of a booster regimen comprises the same amount of RNA as a dose administered in a primary regimen. In some embodiments, at least one dose of a booster regimen is the same as that of the primary regimen. In some embodiments, at least one dose of a booster regimen comprises the same amount of RNA as at least one dose of a primary regimen. In some embodiments, at least one dose of a booster regimen is lower than that of the primary regimen. In some embodiments, at least one dose of a booster regimen comprises an amount of RNA that is lower than that of a primary regimen. In some embodiments, at least one dose of a booster regimen is higher than that of the primary regimen. In some embodiments, at least one dose of a booster regimen comprises an amount of RNA that is higher than that of a primary regimen.

In some embodiments, a booster regimen (e.g., as described herein) is administered to a pediatric patient (e.g., a patient aged 2 through 5 years old, a patient aged 5 through 11 years old, or a patient aged 12 through 15 years old). In some embodiments, a booster regimen is administered to a pediatric patient who is 6 months old to less than 2 years old. In some embodiments, a booster regimen is administered to a pediatric patient who is less than 6 months old. In some embodiments, a booster regimen is administered to a pediatric patient who is 6 months old to less than 5 years old. In some embodiments, a booster regimen is administered to a pediatric patient who is 2 years old to less than 5 years old. In some embodiments, a booster regimen is administered to a pediatric patient who is 5 years old to less than 12 years old. In some embodiments, a booster regimen is administered to a pediatric patient who is 12 years old to less than 16 years old. In some embodiments, each dose of a pediatric booster regimen comprises about 3 μg of RNA. In some embodiments, each dose of a pediatric booster regimen comprises about 10 μg of RNA. In some embodiments, each dose of a pediatric booster regimen comprises about 15 μg of RNA. In some embodiments, each dose of a pediatric booster regimen comprises about 20 μg of RNA. In some embodiments, each dose of a pediatric booster regimen comprises about 25 μg of RNA. In some embodiments, each dose of a pediatric booster regimen comprises about 30 μg of RNA. In some embodiments, a booster regimen is administered to a non-pediatric patient (e.g., a patient 16 years or older, a patient aged 18 through 64 years old, and/or a patient 65 years and older). In some embodiments, each dose of a non-pediatric booster regimen comprises about 3 μg of RNA, about 10 μg of RNA, about 25 μg or RNA, about 30 μg of RNA, about 40 μg of RNA, about 45 μg of RNA, about 50 μg of RNA, about 55 μg of RNA, or about 60 μg of RNA. In some embodiments, the same booster regimen may be administered to both pediatric and non-pediatric patients (e.g., to a patient 12 years or older). In some embodiments, a booster regimen that is administered to a non-pediatric patient is administered in a formulation and dose that is related to that of a primary regimen previously received by the patient by identity with or by dilution as described herein. In some embodiments, a non-pediatric patient who receives a booster regimen at a lower dose than a primary regimen may have experienced an adverse reaction to one or more doses of such primary regimen and/or may have been exposed to and/or infected by SARS-CoV-2 between such primary regimen and such booster regimen, or between doses of such primary regimen and/or of such booster regimen. In some embodiments, pediatric and non-pediatric patients may receive a booster regimen at a higher dose than a primary regimen when waning of vaccine efficacy at lower doses is observed, and/or when immune escape of a variant that is prevalent and/or spreading rapidly at a relevant jurisdiction at the time of administration is observed.

In some embodiments one or more doses of a booster regimen differs from that of a primary regimen. For example, in some embodiments, an administered dose may correspond to a subject's age and a patient may age out of one treatment age group and into a next. Alternatively or additionally, in some embodiments, an administered dose may correspond to a patient's condition (e.g., immunocompromised state) and a different dose may be selected for one or more doses of a booster regimen than for a primary regimen (e.g., due to intervening cancer treatment, infection with HIV, receipt of immunosuppressive therapy, for example associated with an organ transplant. In some embodiments, at least one dose of a booster regimen may comprise an amount of RNA that is higher than at least one dose administered in a primary regimen (e.g., in situations where waning of vaccine efficacy from one or more earlier doses is observed and/or immune escape by a variant (e.g., one described herein) that is prevalent or rapidly spreading is observed in a relevant jurisdiction at the time of administration).

In some embodiments, a primary regimen may involve one or more 3 ug doses and a booster regimen may involve one or more 10 ug doses, and/or one or more 20 ug doses, or one or more 30 ug doses. In some embodiments, a primary regimen may involve one or more 3 ug doses and a booster regimen may involve one or more 3 ug doses. In some embodiments, a primary regimen may involve two or more 3 ug doses (e.g., at least two doses, each comprising 3 ug of RNA, and administered about 21 days after one another) and a booster regimen may involve one or more 3 ug doses. In some embodiments, a primary regimen may involve one or more 10 ug doses and a booster regimen may involve one or more 20 ug doses, and/or one or more 30 ug doses. In some embodiments, a primary regimen may involve one or more 10 ug doses and a booster regimen may involve one or more 10 ug doses. In some embodiments, a primary regimen may involve two or more 10 ug doses (e.g., two doses, each comprising 10 ug of RNA, administered about 21 days apart) and a booster regimen may involve one or more 10 ug doses. In some embodiments, a primary regimen may involve one or more 20 ug doses and a booster regimen may involve one or more 30 ug doses. In some embodiments, a primary regimen may involve one or more 20 ug doses and a booster regimen may involve one or more 20 ug doses. In some embodiments, a primary regimen may involve one or more 30 ug doses, and a booster regimen may also involve one or more 30 ug doses. In some embodiments, a primary regimen may involve two or more 30 ug doses (e.g., two doses, each comprising 30 ug of RNA, administered about 21 days apart), and a booster regimen may also involve one or more 30 ug doses. In some embodiments, a primary regimen may involve two or more 30 ug doses (e.g., two doses, each comprising 30 ug of RNA, administered about 21 days apart), and a booster regimen may involve one or more 50 ug doses. In some embodiments, a primary regimen may involve two or more 30 ug doses (e.g., two doses, each comprising 30 ug of RNA, administered about 21 days apart), and a booster regimen may involve one or more 60 ug doses.

In some embodiments, a subject is administered a booster regimen comprising at least one 30 ug dose of RNA. In some embodiments, a subject is administered a booster regimen comprising at least one 30 ug dose of RNA encoding a SARS-CoV-2 S protein from a Wuhan strain of SARS-CoV-2 (e.g., BNT162b2). In some embodiments, a subject is administered a booster regimen comprising at least one dose of 30 ug of RNA encoding a SARS-CoV-2 S protein having one or more mutations that are characteristic of a SARS-CoV-2 variant (e.g., a variant described herein). In some embodiments, a subject is administered a booster regimen comprising at least one dose of 30 ug of RNA encoding a SARS-CoV-2 S protein having one or more mutations that are characteristic of an Omicron variant (e.g., a BA.1, BA.2, BA.3, BA.4/BA.5, or XBB.1.5 Omicron variant). In some embodiments, a subject is administered a booster regimen comprising at least one dose of 30 ug of RNA, wherein the 30 ug of RNA comprises RNA encoding a SARS-CoV-2 S protein from a Wuhan strain and RNA encoding a SARS-CoV-2 S protein comprising mutations that are characteristic of a SARS-CoV-2 variant (e.g., in some embodiments, a subject is administered a booster regimen comprising at least one dose comprising 15 ug of RNA encoding a SARS-CoV-2 S protein from a Wuhan strain and 15 ug of RNA encoding a SARS-CoV-2 S protein having one or more mutations that are characteristic of an Omicron variant (e.g., a BA.1, BA.4/5, or XBB.1.5 Omicron variant)). In some embodiments, a subject is administered a booster regimen comprising at least one dose comprising 15 ug of RNA encoding a SARS-CoV-2 S protein from a Wuhan strain and 15 ug of RNA encoding a SARS-CoV-2 S protein having one or more mutations that are characteristic of a BA.1 Omicron variant. In some embodiments, a subject is administered a booster regimen comprising at least one dose comprising 10 ug of RNA encoding a SARS-CoV-2 S protein from a Wuhan strain and 20 ug of RNA encoding a SARS-CoV-2 S protein having one or more mutations that are characteristic of a BA.1 Omicron variant. In some embodiments, a subject is administered a booster regimen comprising at least one dose comprising 7.5 ug of RNA encoding a SARS-CoV-2 S protein from a Wuhan strain and 22.5 ug of RNA encoding a SARS-CoV-2 S protein having one or more mutations that are characteristic of a BA.1 Omicron variant. In some embodiments, a subject is administered a booster regimen comprising at least one dose comprising 15 ug of RNA encoding a SARS-CoV-2 S protein from a Wuhan strain and 15 ug of RNA encoding a SARS-CoV-2 S protein having one or more mutations that are characteristic of a BA.2 Omicron variant. In some embodiments, a subject is administered a booster regimen comprising at least one dose comprising 10 ug of RNA encoding a SARS-CoV-2 S protein from a Wuhan strain and 20 ug of RNA encoding a SARS-CoV-2 S protein having one or more mutations that are characteristic of a BA.2 Omicron variant. In some embodiments, a subject is administered a booster regimen comprising at least one dose comprising 7.5 ug of RNA encoding a SARS-CoV-2 S protein from a Wuhan strain and 22.5 ug of RNA encoding a SARS-CoV-2 S protein having one or more mutations that are characteristic of a BA.2 Omicron variant. In some embodiments, a subject is administered a booster regimen comprising at least one dose comprising 15 ug of RNA encoding a SARS-CoV-2 S protein from a Wuhan strain and 15 ug of RNA encoding a SARS-CoV-2 S protein having one or more mutations that are characteristic of a BA.3 Omicron variant. In some embodiments, a subject is administered a booster regimen comprising at least one dose comprising 10 ug of RNA encoding a SARS-CoV-2 S protein from a Wuhan strain and 20 ug of RNA encoding a SARS-CoV-2 S protein having one or more mutations that are characteristic of a BA.3 Omicron variant. In some embodiments, a subject is administered a booster regimen comprising at least one dose comprising 7.5 ug of RNA encoding a SARS-CoV-2 S protein from a Wuhan strain and 22.5 ug of RNA encoding a SARS-CoV-2 S protein having one or more mutations that are characteristic of a BA.3 Omicron variant. In some embodiments, a subject is administered a booster regimen comprising at least one dose comprising 15 ug of RNA encoding a SARS-CoV-2 S protein from a Wuhan strain and 15 ug of RNA encoding a SARS-CoV-2 S protein having one or more mutations that are characteristic of a BA.4 or BA.5 Omicron variant. In some embodiments, a subject is administered a booster regimen comprising at least one dose comprising 10 ug of RNA encoding a SARS-CoV-2 S protein from a Wuhan strain and 20 ug of RNA encoding a SARS-CoV-2 S protein having one or more mutations that are characteristic of a BA.4 or BA.5 Omicron variant. In some embodiments, a subject is administered a booster regimen comprising at least one dose comprising 7.5 ug of RNA encoding a SARS-CoV-2 S protein from a Wuhan strain and 22.5 ug of RNA encoding a SARS-CoV-2 S protein having one or more mutations that are characteristic of a BA.4 or BA.5 Omicron variant.

In some embodiments, a subject is administered a booster regimen comprising at least one dose comprising 15 ug of RNA encoding a SARS-CoV-2 S protein from a BA.1 Omicron variant and 15 ug of RNA encoding a SARS-CoV-2 S protein having one or more mutations that are characteristic of a BA.2 Omicron variant. In some embodiments, a subject is administered a booster regimen comprising at least one dose comprising 10 ug of RNA encoding a SARS-CoV-2 S protein from a BA.1 Omicron variant and 20 ug of RNA encoding a SARS-CoV-2 S protein having one or more mutations that are characteristic of a BA.2 Omicron variant. In some embodiments, a subject is administered a booster regimen comprising at least one dose comprising 7.5 ug of RNA encoding a SARS-CoV-2 S protein from a BA.1 Omicron variant and 22.5 ug of RNA encoding a SARS-CoV-2 S protein having one or more mutations that are characteristic of a BA.2 Omicron variant. In some embodiments, a subject is administered a booster regimen comprising at least one dose comprising 15 ug of RNA encoding a SARS-CoV-2 S protein from a BA.1 Omicron variant and 15 ug of RNA encoding a SARS-CoV-2 S protein having one or more mutations that are characteristic of a BA.3 Omicron variant. In some embodiments, a subject is administered a booster regimen comprising at least one dose comprising 10 ug of RNA encoding a SARS-CoV-2 S protein from a BA.1 Omicron variant and 20 ug of RNA encoding a SARS-CoV-2 S protein having one or more mutations that are characteristic of a BA.3 Omicron variant. In some embodiments, a subject is administered a booster regimen comprising at least one dose comprising 7.5 ug of RNA encoding a SARS-CoV-2 S protein from a BA.1 Omicron variant and 22.5 ug of RNA encoding a SARS-CoV-2 S protein having one or more mutations that are characteristic of a BA.3 Omicron variant. In some embodiments, a subject is administered a booster regimen comprising at least one dose comprising 15 ug of RNA encoding a SARS-CoV-2 S protein from a BA.1 Omicron variant and 15 ug of RNA encoding a SARS-CoV-2 S protein having one or more mutations that are characteristic of a BA.4 or BA.5 Omicron variant. In some embodiments, a subject is administered a booster regimen comprising at least one dose comprising 10 ug of RNA encoding a SARS-CoV-2 S protein from a BA.1 Omicron variant and 20 ug of RNA encoding a SARS-CoV-2 S protein having one or more mutations that are characteristic of a BA.4 or BA.5 Omicron variant. In some embodiments, a subject is administered a booster regimen comprising at least one dose comprising 7.5 ug of RNA encoding a SARS-CoV-2 S protein from a BA.1 Omicron variant and 22.5 ug of RNA encoding a SARS-CoV-2 S protein having one or more mutations that are characteristic of a BA.4 or BA.5 Omicron variant.

In some embodiments, a subject is administered a booster regimen comprising at least one dose comprising 15 ug of RNA encoding a SARS-CoV-2 S protein from a BA.2 Omicron variant and 15 ug of RNA encoding a SARS-CoV-2 S protein having one or more mutations that are characteristic of a BA.4 or BA.5 Omicron variant. In some embodiments, a subject is administered a booster regimen comprising at least one dose comprising 10 ug of RNA encoding a SARS-CoV-2 S protein from a BA.2 Omicron variant and 20 ug of RNA encoding a SARS-CoV-2 S protein having one or more mutations that are characteristic of a BA.4 or BA.5 Omicron variant. In some embodiments, a subject is administered a booster regimen comprising at least one dose comprising 7.5 ug of RNA encoding a SARS-CoV-2 S protein from a BA.2 Omicron variant and 22.5 ug of RNA encoding a SARS-CoV-2 S protein having one or more mutations that are characteristic of a BA.4 or BA.5 Omicron variant. In some embodiments, a subject is administered a booster regimen comprising at least one dose comprising 15 ug of RNA encoding a SARS-CoV-2 S protein from a BA.3 Omicron variant and 15 ug of RNA encoding a SARS-CoV-2 S protein having one or more mutations that are characteristic of a BA.4 or BA.5 Omicron variant. In some embodiments, a subject is administered a booster regimen comprising at least one dose comprising 10 ug of RNA encoding a SARS-CoV-2 S protein from a BA.3 Omicron variant and 20 ug of RNA encoding a SARS-CoV-2 S protein having one or more mutations that are characteristic of a BA.4 or BA.5 Omicron variant. In some embodiments, a subject is administered a booster regimen comprising at least one dose comprising 7.5 ug of RNA encoding a SARS-CoV-2 S protein from a BA.3 Omicron variant and 22.5 ug of RNA encoding a SARS-CoV-2 S protein having one or more mutations that are characteristic of a BA.4 or BA.5 Omicron variant.

In some embodiments, a subject is administered a booster regimen comprising two or more doses of 30 ug of RNA, administered at least two months apart from each other. For example, in some embodiments, subjects are administered a booster regimen comprising two doses of 30 ug of RNA encoding a SARS-CoV-2 S protein having one or more mutations that are characteristic of an Omicron variant (e.g., a BA.1, BA.2, or BA.4, BA.5, or XBB.1.5 Omicron variant).

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 30 ug of RNA encoding a SARS-CoV-2 S protein having one or mutations that are characteristic of an Omicron variant of SARS-CoV-2 (e.g., a BA.1, BA.2, BA.3, BA.4/5, or XBB.1.5 Omicron variant), wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 30 ug dose of RNA comprising 15 ug RNA encoding a SARS-CoV-2 S protein from a Wuhan strain and 15 ug of RNA encoding a SARS-CoV-2 S protein having one or more mutations that are characteristic of an Omicron variant (e.g., a BA.1, BA.4/5, or XBB.1.5 Omicron variant).

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 30 ug of RNA encoding a SARS-CoV-2 S protein having one or mutations that are characteristic of a BA.1 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 30 ug dose of RNA comprising 15 ug RNA encoding a SARS-CoV-2 S protein from a Wuhan strain and 15 ug of RNA encoding a SARS-CoV-2 S protein having one or more mutations that are characteristic of a BA.1 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 30 ug of RNA encoding a SARS-CoV-2 S protein having one or mutations that are characteristic of a BA.2 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 30 ug dose of RNA comprising 15 ug RNA encoding a SARS-CoV-2 S protein from a Wuhan strain and 15 ug of RNA encoding a SARS-CoV-2 S protein having one or more mutations that are characteristic of a BA.2 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 30 ug of RNA encoding a SARS-CoV-2 S protein having one or mutations that are characteristic of a BA.4 or BA.5 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 30 ug dose of RNA comprising 15 ug RNA encoding a SARS-CoV-2 S protein from a Wuhan strain and 15 ug of RNA encoding a SARS-CoV-2 S protein having one or more mutations that are characteristic of a BA.4 or BA.5 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 30 ug dose of RNA comprising 15 ug RNA encoding a SARS-CoV-2 S protein from a BA.1 Omicron variant and 15 ug of RNA encoding a SARS-CoV-2 S protein having one or more mutations that are characteristic of a BA.2 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 30 ug dose of RNA comprising 15 ug RNA encoding a SARS-CoV-2 S protein from a BA.1 Omicron variant and 15 ug of RNA encoding a SARS-CoV-2 S protein having one or more mutations that are characteristic of a BA.4 or BA.5 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 30 ug dose of RNA comprising 15 ug RNA encoding a SARS-CoV-2 S protein from a BA.2 Omicron variant and 15 ug of RNA encoding a SARS-CoV-2 S protein having one or more mutations that are characteristic of a BA.4 or BA.5 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising at least one 30 ug dose of RNA encoding a SARS-CoV-2 S protein from a non-BA.1 Omicron variant.

In some embodiments, a subject is administered a composition comprising one or more first RNAs, each encoding an antigenic polypeptide associated with a coronavirus, and one or more second RNAs, each encoding an antigenic polypeptide associated with an influenza virus, as part of a primary vaccination regimen. In such embodiments, a primary vaccination regimen may comprise two doses, administered about 7 to about 28 days apart. In some embodiments, each dose of the primary vaccination regimen comprises about 30 ug of the one or more first RNAs. In some embodiments, each dose of the primary vaccination regimen comprises about 30 ug of the one or more second RNAs. In some embodiments, each dose of the primary vaccination regimen comprises about 60 ug of the one or more second RNAs.

In some embodiments, a subject is administered a composition comprising one or more first RNAs, each encoding an antigenic polypeptide associated with a coronavirus, and one or more second RNAs, each encoding an antigenic polypeptide associated with an influenza virus, as a booster dose (e.g., to a subject who has previously received one or more doses of a coronavirus vaccine). In such embodiments, a booster dose may be administered at least two months after a previous dose of a coronavirus vaccine. In some embodiments, the booster dose comprises about 30 ug of the one or more first RNAs and about 30 ug of the one or more second RNAs. In some embodiments, the booster dose comprises about 30 ug of the one or more first RNAs and about 60 ug of the one or more second RNAs. In some embodiments, such a booster dose may be administered to a subject who has previously received a single dose of a coronavirus vaccine, two doses of a coronavirus vaccine (e.g., a complete primary vaccination dosing regimen), three doses of a coronavirus vaccine (e.g., a complete primary vaccination dosing regimen and a booster dose), four doses of a coronavirus vaccine (e.g., a complete primary vaccination dosing regimen and two booster doses), or five or more doses of a coronavirus vaccine (e.g., a complete primary vaccination dosing regimen and three or more booster doses). In some embodiments, one of the previously received coronavirus vaccines (e.g., the most recently received coronavirus vaccine) is a bivalent vaccine (e.g., a vaccine comprising an RNA encoding a SARS-CoV-2 S protein of a Wuhan strain and an RNA encoding a SARS-CoV-2 S protein comprising one or more mutations characteristic of a BA.4/5 Omicron variant). In some embodiments, a booster dose is administered to a subject who has previously received four doses of a coronavirus vaccine (e.g., a complete primary vaccination dosing regimen and two booster doses), the most recently received of which was a bivalent vaccine (e.g., a vaccine comprising an RNA encoding a SARS-CoV-2 S protein of a Wuhan strain and an RNA encoding a SARS-CoV-2 S protein comprising one or more mutations characteristic of a BA.4/5 Omicron variant). In some embodiments, a booster dose is administered to a subject who has previously received five doses of a coronavirus vaccine (e.g., a complete primary vaccination dosing regimen and three booster doses), the most recently received of which was a bivalent vaccine (e.g., a vaccine comprising an RNA encoding a SARS-CoV-2 S protein of a Wuhan strain and an RNA encoding a SARS-CoV-2 S protein comprising one or more mutations characteristic of a BA.4/5 Omicron variant).

In some embodiments, a subject is administered (i) a primary regimen comprising two 30 ug doses of RNA encoding a SARS-CoV-2 S protein from a Wuhan strain, wherein the two doses are administered at least approximately 21 days apart, and (ii) a booster regimen comprising at least two 30 ug doses of RNA encoding a SARS-CoV-2 S protein having one or more mutations characteristic of an Omicron variant (e.g., a BA.1, BA.4/5, or XBB.1.5 Omicron variant), wherein the booster regimen is administered at least two months (including, e.g., at least three months, at least four months, at least five months, at least six months, or more) after completion of the primary regimen, and the two booster doses are administered at least two months apart from each other.

In some embodiments, a subject is administered a booster regimen comprising at least one 50 ug dose of RNA. In some embodiments, a subject is administered a booster regimen comprising at least one dose of 50 ug of RNA encoding a SARS-CoV-2 S protein from a Wuhan strain (e.g., BNT162b2). In some embodiments, a subject is administered a booster regimen comprising at least one dose of 50 ug of RNA encoding a SARS-CoV-2 S protein having one or more mutations that are characteristic of a SARS-CoV-2 variant (e.g., a variant described herein). In some embodiments, a subject is administered a booster regimen comprising at least one dose of 50 ug of RNA encoding a SARS-CoV-2 S protein having one or more mutations that are characteristic of an Omicron variant (e.g., a BA.1, BA.4/5, or XBB.1.5 Omicron variant). In some embodiments, a subject is administered a booster regimen comprising at least one 50 ug dose of RNA, wherein the 50 ug of RNA comprises RNA encoding a SARS-CoV-2 S protein from a Wuhan strain and RNA encoding a SARS-CoV-2 S protein comprising mutations that are characteristic of a SARS-CoV-2 variant (e.g., in some embodiments, a subject is administered a booster regimen comprising a 50 ug dose of RNA comprising 25 ug of RNA encoding a SARS-CoV-2 S protein from a Wuhan strain and 25 ug of RNA encoding a SARS-CoV-2 S protein having one or more mutations that are characteristic of an Omicron variant (e.g., a BA.1, BA.4/5, or XBB.1.5 Omicron variant)).

In some embodiments, a subject is administered a booster regimen comprising at least one dose comprising 25 ug of RNA encoding a SARS-CoV-2 S protein from a Wuhan strain and 25 ug of RNA encoding a SARS-CoV-2 S protein having one or more mutations that are characteristic of a BA.1 Omicron variant. In some embodiments, a subject is administered a booster regimen comprising at least one dose comprising 25 ug of RNA encoding a SARS-CoV-2 S protein from a Wuhan strain and 25 ug of RNA encoding a SARS-CoV-2 S protein having one or more mutations that are characteristic of a BA.2 Omicron variant. In some embodiments, a subject is administered a booster regimen comprising at least one dose comprising 25 ug of RNA encoding a SARS-CoV-2 S protein from a Wuhan strain and 25 ug of RNA encoding a SARS-CoV-2 S protein having one or more mutations that are characteristic of a BA.3 Omicron variant. In some embodiments, a subject is administered a booster regimen comprising at least one dose comprising 25 ug of RNA encoding a SARS-CoV-2 S protein from a Wuhan strain and 25 ug of RNA encoding a SARS-CoV-2 S protein having one or more mutations that are characteristic of a BA.4 or BA.5 Omicron variant. In some embodiments, a subject is administered a booster regimen comprising at least one dose comprising 25 ug of RNA encoding a SARS-CoV-2 S protein from a BA.1 Omicron variant and 25 ug of RNA encoding a SARS-CoV-2 S protein having one or more mutations that are characteristic of a BA.2 Omicron variant. In some embodiments, a subject is administered a booster regimen comprising at least one dose comprising 25 ug of RNA encoding a SARS-CoV-2 S protein from a BA.1 Omicron variant and 25 ug of RNA encoding a SARS-CoV-2 S protein having one or more mutations that are characteristic of a BA.3 Omicron variant. In some embodiments, a subject is administered a booster regimen comprising at least one dose comprising 25 ug of RNA encoding a SARS-CoV-2 S protein from a BA.1 Omicron variant and 25 ug of RNA encoding a SARS-CoV-2 S protein having one or more mutations that are characteristic of a BA.4 or BA.5 Omicron variant.

In some embodiments, a subject is administered a booster regimen comprising at least one dose comprising 25 ug of RNA encoding a SARS-CoV-2 S protein from a BA.2 Omicron variant and 25 ug of RNA encoding a SARS-CoV-2 S protein having one or more mutations that are characteristic of a BA.4 or BA.5 Omicron variant. In some embodiments, a subject is administered a booster regimen comprising at least one dose comprising 25 ug of RNA encoding a SARS-CoV-2 S protein from a BA.3 Omicron variant and 25 ug of RNA encoding a SARS-CoV-2 S protein having one or more mutations that are characteristic of a BA.4 or BA.5 Omicron variant.

In some embodiments, a subject is administered (i) a primary regimen comprising two 30 ug doses of RNA encoding a SARS-CoV-2 S protein from a Wuhan strain, wherein the two doses are administered approximately 21 days apart, and (ii) a booster regimen comprising at least one 50 ug dose of RNA encoding a SARS-CoV-2 S protein having one or mutations that are characteristic of an Omicron variant of SARS-CoV-2 (e.g., a BA.1, BA.2, BA.4/5, or XBB.1.5 Omicron variant), wherein the booster regimen is administered at least two months (including, e.g., at least three months, at least four months, at least five months, at least six months, or more) after completion of the primary regimen. In some embodiments, a subject is administered (i) a primary regimen comprising two 30 ug doses of RNA encoding a SARS-CoV-2 S protein from a Wuhan strain, wherein the two doses are administered approximately 21 days apart, and (ii) a booster regimen comprising at least one 50 ug dose of RNA, wherein the 50 ug of RNA comprises 25 ug of RNA encoding a SARS-CoV-2 S protein of a Wuhan strain and 25 ug of RNA encoding a SARS-CoV-2 S protein having one or mutations that are characteristic of an Omicron variant (e.g., a BA.1, BA.2, BA.4/5, or XBB.1.5 variant), wherein the booster regimen is administered at least two months (including, e.g., at least three months, at least four months, at least five months, at least six months, or more) after completion of a first booster regimen.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 50 ug of RNA encoding a SARS-CoV-2 S protein having one or mutations that are characteristic of a BA.1 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 50 ug dose of RNA comprising 25 ug RNA encoding a SARS-CoV-2 S protein from a Wuhan strain and 25 ug of RNA encoding a SARS-CoV-2 S protein having one or more mutations that are characteristic of a BA.1 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 50 ug of RNA encoding a SARS-CoV-2 S protein having one or mutations that are characteristic of a BA.2 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 50 ug dose of RNA comprising 25 ug RNA encoding a SARS-CoV-2 S protein from a Wuhan strain and 25 ug of RNA encoding a SARS-CoV-2 S protein having one or more mutations that are characteristic of a BA.2 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 50 ug of RNA encoding a SARS-CoV-2 S protein having one or mutations that are characteristic of a BA.4 or BA.5 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 50 ug dose of RNA comprising 25 ug RNA encoding a SARS-CoV-2 S protein from a Wuhan strain and 25 ug of RNA encoding a SARS-CoV-2 S protein having one or more mutations that are characteristic of a BA.4 or BA.5 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 50 ug dose of RNA comprising 25 ug RNA encoding a SARS-CoV-2 S protein from a BA.1 Omicron variant and 25 ug of RNA encoding a SARS-CoV-2 S protein having one or more mutations that are characteristic of a BA.2 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 50 ug dose of RNA comprising 25 ug RNA encoding a SARS-CoV-2 S protein from a BA.1 Omicron variant and 25 ug of RNA encoding a SARS-CoV-2 S protein having one or more mutations that are characteristic of a BA.4 or BA.5 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 50 ug dose of RNA comprising 25 ug RNA encoding a SARS-CoV-2 S protein from a BA.2 Omicron variant and 25 ug of RNA encoding a SARS-CoV-2 S protein having one or more mutations that are characteristic of a BA.4 or BA.5 Omicron variant.

In some embodiments, a subject is administered a booster regimen comprising at least one 60 ug dose of RNA. In some embodiments, a subject is administered a booster regimen comprising 60 ug of RNA encoding a SARS-CoV-2 S protein from a Wuhan variant. In some embodiments, a subject is administered 60 ug of RNA encoding a SARS-CoV-2 S protein having one or more mutations that are characteristic of a SARS-CoV-2 variant (e.g., a variant described herein). In some embodiments, a subject is administered a booster regimen comprising 60 ug of RNA encoding a SARS-CoV-2 S protein having one or more mutations that are characteristic of an Omicron variant (e.g., a BA.1, BA.2, BA.4/5, or XBB.1.5 Omicron variant). In some embodiments, a subject is administered a booster regimen comprising 60 ug of RNA, wherein the RNA comprises a first RNA encoding a SARS-CoV-2 S protein from a Wuhan strain, and at least one additional RNA encoding a SARS-CoV-2 S protein comprising mutations that are characteristic of a SARS-CoV-2 variant (e.g., in some embodiments, a subject is administered a booster regimen comprising 30 ug of RNA encoding a SARS-CoV-2 S protein from a Wuhan strain and 30 ug of RNA encoding a SARS-CoV-2 S protein having one or more mutations that are characteristic of an Omicron variant (e.g., a BA.1, BA.2, BA.4/5, or XBB.1.5 variant).

In some embodiments, a subject is administered a booster regimen comprising at least one dose comprising 30 ug of RNA encoding a SARS-CoV-2 S protein from a Wuhan strain and 30 ug of RNA encoding a SARS-CoV-2 S protein having one or more mutations that are characteristic of a BA.1 Omicron variant. In some embodiments, a subject is administered a booster regimen comprising at least one dose comprising 20 ug of RNA encoding a SARS-CoV-2 S protein from a Wuhan strain and 40 ug of RNA encoding a SARS-CoV-2 S protein having one or more mutations that are characteristic of a BA.1 Omicron variant. In some embodiments, a subject is administered a booster regimen comprising at least one dose comprising 15 ug of RNA encoding a SARS-CoV-2 S protein from a Wuhan strain and 45 ug of RNA encoding a SARS-CoV-2 S protein having one or more mutations that are characteristic of a BA.1 Omicron variant. In some embodiments, a subject is administered a booster regimen comprising at least one dose comprising 30 ug of RNA encoding a SARS-CoV-2 S protein from a Wuhan strain and 30 ug of RNA encoding a SARS-CoV-2 S protein having one or more mutations that are characteristic of a BA.2 Omicron variant. In some embodiments, a subject is administered a booster regimen comprising at least one dose comprising 20 ug of RNA encoding a SARS-CoV-2 S protein from a Wuhan strain and 40 ug of RNA encoding a SARS-CoV-2 S protein having one or more mutations that are characteristic of a BA.2 Omicron variant. In some embodiments, a subject is administered a booster regimen comprising at least one dose comprising 15 ug of RNA encoding a SARS-CoV-2 S protein from a Wuhan strain and 45 ug of RNA encoding a SARS-CoV-2 S protein having one or more mutations that are characteristic of a BA.2 Omicron variant. In some embodiments, a subject is administered a booster regimen comprising at least one dose comprising 30 ug of RNA encoding a SARS-CoV-2 S protein from a Wuhan strain and 30 ug of RNA encoding a SARS-CoV-2 S protein having one or more mutations that are characteristic of a BA.3 Omicron variant. In some embodiments, a subject is administered a booster regimen comprising at least one dose comprising 20 ug of RNA encoding a SARS-CoV-2 S protein from a Wuhan strain and 40 ug of RNA encoding a SARS-CoV-2 S protein having one or more mutations that are characteristic of a BA.3 Omicron variant. In some embodiments, a subject is administered a booster regimen comprising at least one dose comprising 15 ug of RNA encoding a SARS-CoV-2 S protein from a Wuhan strain and 45 ug of RNA encoding a SARS-CoV-2 S protein having one or more mutations that are characteristic of a BA.3 Omicron variant. In some embodiments, a subject is administered a booster regimen comprising at least one dose comprising 30 ug of RNA encoding a SARS-CoV-2

S protein from a Wuhan strain and 30 ug of RNA encoding a SARS-CoV-2 S protein having one or more mutations that are characteristic of a BA.4 or BA.5 Omicron variant. In some embodiments, a subject is administered a booster regimen comprising at least one dose comprising 20 ug of RNA encoding a SARS-CoV-2 S protein from a Wuhan strain and 40 ug of RNA encoding a SARS-CoV-2 S protein having one or more mutations that are characteristic of a BA.4 or BA.5 Omicron variant. In some embodiments, a subject is administered a booster regimen comprising at least one dose comprising 15 ug of RNA encoding a SARS-CoV-2 S protein from a Wuhan strain and 45 ug of RNA encoding a SARS-CoV-2 S protein having one or more mutations that are characteristic of a BA.4 or BA.5 Omicron variant.

In some embodiments, a subject is administered a booster regimen comprising at least one dose comprising 30 ug of RNA encoding a SARS-CoV-2 S protein from a BA.1 Omicron variant and 30 ug of RNA encoding a SARS-CoV-2 S protein having one or more mutations that are characteristic of a BA.2 Omicron variant. In some embodiments, a subject is administered a booster regimen comprising at least one dose comprising 20 ug of RNA encoding a SARS-CoV-2 S protein from a BA.1 Omicron variant and 40 ug of RNA encoding a SARS-CoV-2 S protein having one or more mutations that are characteristic of a BA.2 Omicron variant. In some embodiments, a subject is administered a booster regimen comprising at least one dose comprising 15 ug of RNA encoding a SARS-CoV-2 S protein from a BA.1 Omicron variant and 45 ug of RNA encoding a SARS-CoV-2 S protein having one or more mutations that are characteristic of a BA.2 Omicron variant. In some embodiments, a subject is administered a booster regimen comprising at least one dose comprising 30 ug of RNA encoding a SARS-CoV-2 S protein from a BA.1 Omicron variant and 30 ug of RNA encoding a SARS-CoV-2 S protein having one or more mutations that are characteristic of a BA.3 Omicron variant. In some embodiments, a subject is administered a booster regimen comprising at least one dose comprising 20 ug of RNA encoding a SARS-CoV-2 S protein from a BA.1 Omicron variant and 40 ug of RNA encoding a SARS-CoV-2 S protein having one or more mutations that are characteristic of a BA.3 Omicron variant. In some embodiments, a subject is administered a booster regimen comprising at least one dose comprising 15 ug of RNA encoding a SARS-CoV-2 S protein from a BA.1 Omicron variant and 45 ug of RNA encoding a SARS-CoV-2 S protein having one or more mutations that are characteristic of a BA.3 Omicron variant. In some embodiments, a subject is administered a booster regimen comprising at least one dose comprising 30 ug of RNA encoding a SARS-CoV-2 S protein from a BA.1 Omicron variant and 30 ug of RNA encoding a SARS-CoV-2 S protein having one or more mutations that are characteristic of a BA.4 or BA.5 Omicron variant. In some embodiments, a subject is administered a booster regimen comprising at least one dose comprising 20 ug of RNA encoding a SARS-CoV-2 S protein from a BA.1 Omicron variant and 40 ug of RNA encoding a SARS-CoV-2 S protein having one or more mutations that are characteristic of a BA.4 or BA.5 Omicron variant. In some embodiments, a subject is administered a booster regimen comprising at least one dose comprising 15 ug of RNA encoding a SARS-CoV-2 S protein from a BA.1 Omicron variant and 45 ug of RNA encoding a SARS-CoV-2 S protein having one or more mutations that are characteristic of a BA.4 or BA.5 Omicron variant.

In some embodiments, a subject is administered a booster regimen comprising at least one dose comprising 30 ug of RNA encoding a SARS-CoV-2 S protein from a BA.2 Omicron variant and 30 ug of RNA encoding a SARS-CoV-2

S protein having one or more mutations that are characteristic of a BA.4 or BA.5 Omicron variant. In some embodiments, a subject is administered a booster regimen comprising at least one dose comprising 20 ug of RNA encoding a SARS-CoV-2 S protein from a BA.2 Omicron variant and 40 ug of RNA encoding a SARS-CoV-2 S protein having one or more mutations that are characteristic of a BA.4 or BA.5 Omicron variant. In some embodiments, a subject is administered a booster regimen comprising at least one dose comprising 15 ug of RNA encoding a SARS-CoV-2 S protein from a BA.2 Omicron variant and 45 ug of RNA encoding a SARS-CoV-2 S protein having one or more mutations that are characteristic of a BA.4 or BA.5 Omicron variant. In some embodiments, a subject is administered a booster regimen comprising at least one dose comprising 30 ug of RNA encoding a SARS-CoV-2 S protein from a BA.3 Omicron variant and 30 ug of RNA encoding a SARS-CoV-2 S protein having one or more mutations that are characteristic of a BA.4 or BA.5 Omicron variant. In some embodiments, a subject is administered a booster regimen comprising at least one dose comprising 20 ug of RNA encoding a SARS-CoV-2 S protein from a BA.3 Omicron variant and 40 ug of RNA encoding a SARS-CoV-2 S protein having one or more mutations that are characteristic of a BA.4 or BA.5 Omicron variant. In some embodiments, a subject is administered a booster regimen comprising at least one dose comprising 15 ug of RNA encoding a SARS-CoV-2 S protein from a BA.3 Omicron variant and 45 ug of RNA encoding a SARS-CoV-2 S protein having one or more mutations that are characteristic of a BA.4 or BA.5 Omicron variant.

In some embodiments, a subject is administered (i) a primary regimen comprising two 30 ug doses of RNA encoding a SARS-CoV-2 S protein from a Wuhan strain of SARS-CoV-2, wherein the two doses are administered approximately 21 days apart, and (ii) a booster regimen comprising at least one 60 ug dose of RNA encoding a SARS-CoV-2 S protein from a Wuhan strain of SARS-CoV-2, wherein the booster regimen is administered at least two months (including, e.g., at least three months, at least four months, at least five months, at least six months, or more) after completion of the primary regimen.

In some embodiments, a subject is administered (i) a primary regimen comprising two 30 ug doses of RNA encoding a SARS-CoV-2 S protein from a Wuhan strain, wherein the two doses are administered approximately 21 days apart, and (ii) a booster regimen comprising at least one 60 ug dose of RNA encoding a SARS-CoV-2 S protein having one or mutations that are characteristic of an Omicron variant of SARS-CoV-2 (e.g., a BA.1, BA.2, BA.4/5, or XBB.1.5 Omicron variant), wherein the booster regimen is administered at least two months (including, e.g., at least three months, at least four months, at least five months, at least six months, or more) after completion of the primary regimen. In some embodiments, a subject is administered (i) a primary regimen comprising two 30 ug doses of RNA encoding a SARS-CoV-2 S protein from a Wuhan strain, wherein the two doses are administered approximately 21 days apart, and (iii) a booster regimen comprising at least one 60 ug dose of RNA comprising 30 ug of RNA encoding a SARS-CoV-2 S protein having one or mutations that are characteristic of an Omicron variant of SARS-CoV-2 (e.g., a BA.1, BA.4/5, or XBB.1.5 Omicron variant) and 30 ug of RNA encoding a SARS-CoV-2 S protein from a Wuhan strain, wherein a second booster regimen is administered at least two months (including, e.g., at least three months, at least four months, at least five months, at least six months, or more) after completion of a first booster regimen.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 60 ug of RNA encoding a SARS-CoV-2 S protein having one or mutations that are characteristic of a BA.1 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 60 ug dose of RNA comprising 30 ug RNA encoding a SARS-CoV-2 S protein from a Wuhan strain and 30 ug of RNA encoding a SARS-CoV-2 S protein having one or more mutations that are characteristic of a BA.1 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 60 ug of RNA encoding a SARS-CoV-2 S protein having one or mutations that are characteristic of a BA.2 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 60 ug dose of RNA comprising 30 ug RNA encoding a SARS-CoV-2 S protein from a Wuhan strain and 30 ug of RNA encoding a SARS-CoV-2 S protein having one or more mutations that are characteristic of a BA.2 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a dose of 60 ug of RNA encoding a SARS-CoV-2 S protein having one or mutations that are characteristic of a BA.4 or BA.5 Omicron variant of SARS-CoV-2, wherein the booster regimen is administered at least two months (including, e.g., at least three months, at least four months, at least five months, at least six months, or more) after completion of the primary regimen. In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 60 ug dose of RNA comprising 30 ug RNA encoding a SARS-CoV-2 S protein from a Wuhan strain and 30 ug of RNA encoding a SARS-CoV-2 S protein having one or more mutations that are characteristic of a BA.4/5 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 60 ug dose of RNA comprising 30 ug RNA encoding a SARS-CoV-2 S protein from a BA.1 Omicron variant and 30 ug of RNA encoding a SARS-CoV-2 S protein having one or more mutations that are characteristic of a BA.2 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 60 ug dose of RNA comprising 30 ug RNA encoding a SARS-CoV-2 S protein from a BA.1 Omicron variant and 30 ug of RNA encoding a SARS-CoV-2 S protein having one or more mutations that are characteristic of a BA.4 or BA.5 Omicron variant.

In some embodiments, a subject is administered (i) a primary regimen comprising at least two 30 ug doses of RNA encoding a SARS-CoV-2 S protein from a Wuhan strain, wherein the two doses are administered at least approximately 21 days apart, and (ii) a booster regimen comprising a 60 ug dose of RNA comprising 30 ug RNA encoding a SARS-CoV-2 S protein from a BA.2 Omicron variant and 30 ug of RNA encoding a SARS-CoV-2 S protein having one or more mutations that are characteristic of a BA.4 or BA.5 Omicron variant.

In some embodiments, a patient is administered a primary regimen comprising two 30 ug doses, administered approximately 21 days apart, and a booster regimen comprising at least one 60 ug dose of RNA (e.g., 60 ug of RNA encoding a SARS-CoV-2 S protein from a Wuhan strain, 60 ug of RNA encoding a SARS-CoV-2 S protein having one or more mutations that are characteristic of an Omicron variant (e.g., a BA.1, BA.2, BA.4/5, or XBB.1.5 Omicron variant), or 30 ug of RNA encoding a SARS-CoV-2 S protein from a Wuhan strain and 30 ug of RNA encoding a SARS-CoV-2 S protein having one or more mutations that are characteristic of an Omicron variant (e.g., a BA.1, BA.4/5, or XBB.1.5 Omicron variant)). In some embodiments, a patient is administered a primary regimen comprising two 30 ug doses, administered approximately 21 days apart, and a booster regimen comprising at least one 50 ug dose of RNA (e.g., 50 ug of RNA encoding a SARS-CoV-2 S protein from a Wuhan strain, 50 ug of RNA encoding a SARS-CoV-2 S protein having one or more mutations that are characteristic of an Omicron variant (e.g., a BA.1, BA.4/5, or XBB.1.5 Omicron variant), or 25 ug of RNA encoding a SARS-CoV-2 S protein from a Wuhan strain and 25 ug of RNA encoding a SARS-CoV-2 S protein having one or more mutations that are characteristic of an Omicron variant (e.g., a BA.1, BA.4/5, or XBB.1.5 Omicron variant)). In some embodiments, a patient is administered a primary regimen comprising two 30 ug doses, administered approximately 21 days apart, and a booster regimen comprising at least one 30 ug dose of RNA (e.g., 30 ug of RNA encoding a SARS-CoV-2 S protein from a Wuhan strain, 30 ug of RNA encoding a SARS-CoV-2 S protein having one or more mutations that are characteristic of an Omicron variant (e.g., a BA.1, BA.4/5, or XBB.1.5 Omicron variant), or 15 ug of RNA encoding a SARS-CoV-2 S protein from a Wuhan strain and 15 ug of RNA encoding a SARS-CoV-2 S protein having one or more mutations that are characteristic of an Omicron variant (e.g., a BA.1, BA.4/5, or XBB.1.5 Omicron variant)).

In some embodiments, a primary regimen may involve one or more 30 ug doses and a booster regimen may involve one or more 20 ug doses, one or more 10 ug doses, and/or one or more 3 ug doses. In some embodiments, a primary regimen may involve one or more 20 ug doses and a booster regimen may involve one or more 10 ug doses, and/or one or more 3 ug doses. In some embodiments, a primary regimen may involve one or more 10 ug doses and a booster regimen may involve one or more 3 ug doses. In some embodiments, a primary regimen may involve one or more 3 ug doses, and a booster regimen may also involve one or more 3 ug doses.

In some embodiments, a booster regimen comprises a single dose, e.g., for patients who experienced an adverse reaction while receiving the primary regimen.

In some embodiments, the same RNA as used in a primary regimen is used in a booster regimen. In some embodiment, an RNA used in primary and booster regimens is BNT162b2.

In some embodiments, a different RNA is used in a booster regimen relative to that used in a primary regimen administered to the same subject. In some embodiments, BNT162b2 is used in a primary regimen but not in a booster regimen. In some embodiments, BNT162b2 is used in a booster regimen but not in a primary regimen. In some embodiments, a similar BNT162b2 construct can be used in a primary regimen and in a booster regimen, except that the RNA constructs used in the primary and booster regimens encode a SARS-CoV-2 S protein (or an immunogenic portion thereof) of different SARS-CoV-2 strains (e.g., as described herein).

In some embodiments, where BNT162b2 is used for a primary regimen or a booster regimen but not both, and a different RNA is used in the other, such different RNA may be an RNA encoding the same SARS-CoV-2 S protein but with different codon optimization or other different RNA sequence. In some embodiments, such different RNA may encode a SARS-CoV-2 S protein (or an immunogenic portion thereof) of a different SARS-CoV-2 strain, e.g., of a variant strain discussed herein. In some such embodiments, such variant strain that is prevalent or rapidly spreading in a relevant jurisdiction. In some embodiments, such different RNA may be an RNA encoding a SARS-CoV-2 S protein or variant thereof (or immunogenic portion of either) comprising one or more mutations described herein for S protein variants such as SARS-CoV-2 S protein variants, in particular naturally occurring S protein variants; in some such embodiments, a SARS-CoV-2 variant may be selected from the group consisting of VOC-202012/01, 501.V2, Cluster 5 and B.1.1.248. In some embodiments, a SARS-CoV-2 variant may be selected from the group consisting of VOC-202012/01, 501.V2, Cluster 5 and B.1.1.248, B.1.1.7, B.1.617.2, and B.1.1.529. In some embodiments, a booster regimen comprises at least one dose of RNA that encodes a SARS-CoV-2 S protein (or an immunogenic fragment thereof) of a variant that is spreading rapidly in a relevant jurisdiction at the time of administration. In some such embodiments, a variant that is encoded by RNA administered in a booster regimen may be different from that encoded by RNA administered in a primary regimen.

In some embodiments, a booster regimen comprises administering (i) a dose of RNA encoding the same SARS-CoV-2 S protein (or an immunogenic fragment thereof) as the RNA administered in the primary regimen (e.g., an RNA encoding a SARS-CoV-2 the S protein (or an immunogenic fragment thereof) from the SARS-CoV-2 Wuhan strain) and (ii) a dose of RNA encoding a SARS-CoV-2 S protein (or an immunogenic fragment thereof) of a variant that is spreading rapidly in a relevant jurisdiction at the time of administration (e.g., a SARS-CoV-2 S protein (or an immunogenic fragment thereof) from one of the SARS-CoV-2 variants discussed herein).

In some embodiments, a booster regimen comprises multiple doses (e.g., at least two doses, at least three doses, or more). For example, in some embodiments, a first dose of a booster regimen may comprise an RNA encoding the same SARS-CoV-2 S protein (or an immunogenic fragment thereof) administered in the primary regimen and a second dose of a booster regimen may comprise the RNA encoding a SARS-CoV-2 S protein of a variant that is spreading rapidly in a relevant jurisdiction at the time of administration. In some embodiments, a first dose of a booster regimen may comprise RNA encoding a SARS-CoV-2 S protein (or an immunogenic fragment thereof) of a variant that is spreading rapidly in a relevant jurisdiction at the time of administration and a second dose of a booster regimen may comprise RNA encoding the same SARS-CoV-2 S protein (or an immunogenic fragment thereof) administered in the primary regimen. In some embodiments, the booster regimen comprises multiple doses, and the RNA encoding the S protein of a variant that is spreading rapidly in a relevant jurisdiction is administered in a first dose and the RNA encoding the S protein administered in the primary regimen is administered in a second dose.

In some embodiments, doses (e.g., a first and a second dose or any two consecutive doses) in a booster regimen are administered at least 2 weeks apart, including, e.g., at least 3 weeks, at least 4 weeks, at least 5 weeks, at least 6 weeks, at least 7 week, at least 8 weeks, at least 9 weeks, at least 10 weeks, at least 11 weeks, at least 12 weeks, at least 13 weeks, at least 14 weeks, at least 15 weeks, at least 16 weeks, or longer, apart. In some embodiments, doses (e.g., a first and a second dose or any two consecutive doses) in a booster regimen are administered approximately 2 to 168 weeks apart. In some embodiments, doses (e.g., a first and a second dose or any two consecutive doses) in a booster regimen are administered approximately 3 to 12 weeks apart. In some embodiments, doses (e.g., a first and a second dose or any two consecutive doses) in a booster regimen are administered approximately 4 to 10 weeks apart. In some embodiments, doses (e.g., a first and a second dose or any two consecutive doses) in a booster regimen are administered approximately 6 to 8 weeks apart. (e.g., about 21 days apart, or about 6 to 8 weeks apart). In some embodiments, the first and second dose are administered on the same day (e.g., by intramuscular injection at different sites on the subject).

In such embodiments, the booster regimen can optionally further comprise a third and fourth dose, administered approximately 2 to 8 weeks after the first and second dose (e.g., about 21 days after the first and second dose, or about 6 weeks to about 8 weeks after the first and second dose), where the third and fourth dose are also administered on the same day (e.g., by intramuscular injection at different sites on the subject), and comprise the same RNAs administered in the first and second doses of the booster regimen.

In some embodiments, multiple booster regimens may be administered. In some embodiments, a booster regimen is administered to a patient who has previously been administered a booster regimen.

In some embodiments, a second booster regimen is administered to a patient who has previously received a first booster regimen, and the amount of RNA administered in at least one dose of a second booster regimen is higher than the amount of RNA administered in at least one dose of a first booster regimen.

In some embodiments, a second booster regimen comprises administering at least one dose of 3 ug of RNA. In some embodiments, a second booster regimen comprises administering at least one dose of 5 ug of RNA. In some embodiments, a second booster regimen comprises administering at least one dose of 10 ug of RNA. In some embodiments, a second booster regimen comprises administering at least one dose of 15 ug of RNA. In some embodiments, a second booster regimen comprises administering at least one dose of 20 ug of RNA. In some embodiments, a second booster regimen comprises administering at least one dose of 25 ug of RNA. In some embodiments, a second booster regimen comprises administering at least one dose of 30 ug of RNA. In some embodiments, a second booster regimen comprises administering at least one dose of 50 ug of RNA. In some embodiments, a second booster regimen comprises administering at least one dose of 60 ug of RNA.

In some embodiments, a subject is administered a primary regimen that comprises two doses of 30 ug of RNA, administered approximately 21 days apart, and a booster regimen comprising at least one dose of approximately 30 ug of RNA. In some embodiments, a subject is administered a primary regimen that comprises two doses of 30 ug of RNA, administered approximately 21 days apart, and a booster regimen comprising at least one dose of approximately 50 ug of RNA. In some embodiments, a subject is administered a primary regimen that comprises two doses of 30 ug of RNA, administered approximately 21 days apart, and a booster regimen comprising at least one dose of approximately 60 ug of RNA.

In some embodiments, a subject is administered a primary regimen that comprises two doses of 30 ug of RNA, administered approximately 21 days apart, a first booster regimen comprising at least one dose of approximately 30 ug of RNA, and a second booster regimen comprising at least one dose of approximately 30 ug of RNA. In some embodiments, a subject is administered a primary regimen that comprises two doses of 30 ug of RNA, administered approximately 21 days apart, a first booster regimen comprising at least one dose of approximately 30 ug of RNA, and a second booster regimen comprising at least one dose of approximately 50 ug of RNA. In some embodiments, a subject is administered a primary regimen that comprises two doses of 30 ug of RNA, administered approximately 21 days apart, a first booster regimen comprising at least one dose of approximately 30 ug of RNA, and a second booster regimen comprising at least one dose of approximately 60 ug of RNA. In some embodiments, a first booster regimen comprises two doses of RNA, wherein each dose comprises an RNA encoding a Spike protein from a different SARS-CoV-2 variant. In some embodiments, a first booster regimen comprises two doses of RNA, wherein each dose comprises an RNA encoding a Spike protein from a different SARS-CoV-2 variant, and wherein the two doses of RNA are administered on the same day. In some embodiments, the two doses of RNA are administered in a single composition (e.g., by mixing a first composition comprising an RNA encoding a Spike protein from a first SARS-CoV-2 variant with a second composition comprising an RNA encoding a Spike protein from a second SARS-CoV-2 variant).

In some embodiments, a subject is administered a booster regimen comprising a first dose comprising an RNA that encodes a Spike protein from a Wuhan strain of SARS-CoV-2 and a second dose comprising an RNA that encodes a Spike protein comprising mutations from a variant that is prevalent and/or rapidly spreading in a relevant jurisdiction at the time of administering the booster regimen, wherein the first dose and the second dose of RNA may be administered on the same day. In some embodiments, a subject is administered a booster regimen comprising a first dose comprising an RNA that encodes a Spike protein from a Wuhan strain of SARS-CoV-2 and a second dose comprising an RNA that encodes a Spike protein comprising mutations from an alpha variant of SARS-CoV-2, wherein the first dose and the second dose may be administered on the same day. In some embodiments, a subject is administered a booster regimen comprising a first dose comprising an RNA that encodes a Spike protein from a Wuhan strain of SARS-CoV-2 and a second dose comprising an RNA that encodes a Spike protein comprising mutations from a beta variant of SARS-CoV-2, wherein the first dose and the second dose may be administered on the same day. In some embodiments, a subject is administered a booster regimen comprising a first dose comprising an RNA that encodes a Spike protein from a Wuhan strain of SARS-CoV-2 and a second dose comprising an RNA that encodes a Spike protein comprising mutations from a delta variant of SARS-CoV-2, wherein the first dose and the second dose may be administered on the same day. In some embodiments, a subject is administered a booster regimen comprising a first dose comprising an RNA that encodes a Spike protein from a Wuhan strain of SARS-CoV-2 and a second dose comprising an RNA that encodes a Spike protein comprising mutations from an Omicron variant of SARS-CoV-2 (e.g., a BA.1, BA.4/5, or XBB.1.5 Omicron variant), wherein the first dose and the second dose may be administered on the same day. Such booster regimens may be administered, e.g., to a subject previously administered a primary dosing regimen and/or to a subject previously administered a primary dosing regimen and a booster regimen.

In some embodiments, a subject is administered a first booster regimen comprising a first dose of 15 ug of RNA encoding a Spike protein from a Wuhan variant and a second dose of 15 ug of RNA encoding a Spike protein from an Omicron variant of SARS-CoV-2 (e.g., a BA.1, BA.4/5, or XBB.1.5 Omicron variant), where the first and the second dose are administered on the same day (e.g., wherein compositions comprising the RNA are mixed prior to administration, and the mixture is then administered to a patient). In some embodiments, a subject is administered a first booster regimen comprising a first dose of 25 ug of RNA encoding a Spike protein from a Wuhan variant and a second dose of 25 ug of RNA encoding a Spike protein from an Omicron variant of SARS-CoV-2 (e.g., a BA.1,

BA.4/5, or XBB.1.5 Omicron variant). In some embodiments, the first and the second doses are optionally administered on the same day. In some embodiments, a subject is administered a first booster regimen comprising a first dose of 25 ug of RNA encoding a Spike protein from a Wuhan variant and a second dose of 25 ug of RNA encoding a Spike protein from an Omicron variant of SARS-CoV-2 (e.g., a BA.1, BA.4/5, or XBB.1.5 Omicron variant). In some embodiments, the first and the second doses are administered on the same day. In some embodiments, a subject is administered a first booster regimen comprising a first dose of 30 ug of RNA encoding a Spike protein from a Wuhan variant and a second dose of 30 ug of RNA encoding a Spike protein from an Omicron variant of SARS-CoV-2 (e.g., a BA.1, BA.4/5, or XBB.1.5 Omicron variant), wherein the first and the second dose are optionally administered on the same day (e.g., in separate administrations or as administration of a multivalent vaccine). In some embodiments, such a first booster regimen is administered to a subject previously administered a primary regimen comprising two doses of 30 ug of RNA, administered about 21 days apart wherein the first booster regimen is administered at least 3 months (e.g., at least 4, at least 5, or at least 6 months) after administration of a primary regimen.

In some embodiments, a subject is administered a second booster regimen comprising a first dose of 15 ug of RNA encoding a Spike protein from a Wuhan variant and a second dose of 15 ug of RNA encoding a Spike protein from an Omicron variant of SARS-CoV-2 (e.g., a BA.1, BA.4/5, or XBB.1.5 Omicron variant), where the first and the second dose are administered on the same day (e.g., wherein compositions comprising the RNA are mixed prior to administration to form a multivalent vaccine, and the mixture is then administered to a patient). In some embodiments, a subject is administered a second booster regimen comprising a first dose of 25 ug of RNA encoding a Spike protein from a Wuhan variant and a second dose of 25 ug of RNA encoding a Spike protein from an Omicron variant of SARS-CoV-2 (e.g., a BA.1, BA.4/5, or XBB.1.5 Omicron variant), wherein the first dose and the second dose are optionally administered on the same day (e.g., via administration of a multivalent vaccine or via administration of separate compositions). In some embodiments, a subject is administered a second booster regimen comprising a first dose of 25 ug of RNA encoding a Spike protein from a Wuhan variant and a second dose of 25 ug of RNA encoding a Spike protein from an Omicron variant of SARS-CoV-2 (e.g., a BA.1, BA.4/5, or XBB.1.5 Omicron variant). In some embodiments, a subject is administered a second booster regimen comprising a first dose of 30 ug of RNA encoding a Spike protein from a Wuhan variant and a second dose of 30 ug of RNA encoding a Spike protein from an Omicron variant of SARS-CoV-2 (e.g., a BA.1, BA.4/5, or XBB.1.5 Omicron variant), wherein the first dose and the second dose are optionally administered on the same day (e.g., via administration of a multivalent vaccine or via administration of separate compositions). In some embodiments, such a second booster regimen is administered to a subject previously administered a primary regimen comprising two doses of 30 ug of RNA, administered about 21 days apart. In some embodiments, such a second booster regimen is administered to a subject previously administered a primary regimen comprising two doses of 30 ug of RNA, administered about 21 days apart, and a first booster regimen comprising a dose of 30 ug of RNA, wherein the second booster regimen is administered at least 3 months (e.g., at least 4, at least 5, or at least 6 months) after administration of a first booster regimen.

In some embodiments, patients receiving dose(s) of RNA compositions as described herein are monitored for one or more particular conditions, e.g., following administration of one or more doses. In some embodiments, such condition(s) may be or comprise allergic reaction(s) (particularly in subject(s) with a history of relevant allergies or allergic reactions), myocarditis (inflammation of the heart muscle, particularly where the subject is a young male and/or may have experienced prior such inflammation), pericarditis (inflammation of the lining outside the heart, particularly where the subject is a young males and/or may have experienced prior such inflammation), fever, bleeding (particularly where the subject is known to have a bleeding disorder or to be receiving therapy with a blood thinner). Alternatively or additionally, patients who may receive closer monitoring may be or include patients who are immunocompromised or are receiving therapy with a medicine that affects the immune system, are pregnant or planning to become pregnant, are breastfeeding, have received another COVID-19 vaccine and/or another influenza vaccine, and/or have ever fainted in association with an injection. In some embodiments, patients are monitored for myocarditis following administration of one of the compositions disclosed herein. In some embodiments, patients are monitored for pericarditis following administration of one of the compositions disclosed herein. Patients may be monitored and/or treated for the condition using current standards of care.

In some embodiments, efficacy for mRNA compositions described in pediatric populations (e.g., described herein) may be assessed by various metrics described herein (including, e.g., but not limited to COVID-19 incidence per 1000 person-years in subjects with no serological or virological evidence of past SARS-CoV-2 infection, or COVID-19 and/or influenza incidence per 1000 person-years in subjects regardless of evidence of past infection; geometric mean ratio (GMR) of SARS COV-2 neutralizing titers and/or influenza virus neutralizing titers measured, e.g., 7 days after a second dose; etc.)

In some embodiments, pediatric populations described herein (e.g., from 12 to less than 16 years of age) may be monitored for occurrence of multisystem inflammatory syndrome (MIS) (e.g., inflammation in different body parts such as, e.g., heart, lung, kidneys, brain, skin, eyes, and/or gastrointestinal organs), after administration of an RNA composition (e.g., mRNA) described herein. Exemplary symptoms of MIS in children may include, but are not limited to fever, abdominal pain, vomiting, diarrhea, neck pain, rash, bloodshot eyes, feeling extra tried, and combinations thereof.

In one embodiment, RNA administered as described above is nucleoside modified messenger RNA (modRNA) described herein as BNT162b1 (RBP020.3), BNT162b2 (RBP020.1 or RBP020.2), or BNT162b3 (e.g., BNT162b3c). In one embodiment, RNA administered as described above is nucleoside modified messenger RNA (modRNA) described herein as RBP020.2. In one embodiment, RNA encoding a vaccine antigen is nucleoside modified messenger RNA (modRNA) described herein as BNT162b3 (e.g., BNT162b3c).

In one embodiment, RNA administered as described above is nucleoside modified messenger RNA (modRNA) and (i) comprises the nucleotide sequence of SEQ ID NO: 21, a nucleotide sequence having at least 99%, 98%, 97%, 96%, 95%, 90%, 85%, or 80% identity to the nucleotide sequence of SEQ ID NO: 21, and/or (ii) encodes an amino acid sequence comprising the amino acid sequence of SEQ ID NO: 5, or an amino acid sequence having at least 99%, 98%, 97%, 96%, 95%, 90%, 85%, or 80% identity to the amino acid sequence of SEQ ID NO: 5. In one embodiment, RNA administered as described above is nucleoside modified messenger RNA (modRNA) and (i) comprises the nucleotide sequence of SEQ ID NO: 21; and/or (ii) encodes an amino acid sequence comprising the amino acid sequence of SEQ ID NO: 5.

In one embodiment, RNA administered as described above is nucleoside modified messenger RNA (modRNA) and (i) comprises the nucleotide sequence of SEQ ID NO: 19, or 20, a nucleotide sequence having at least 99%, 98%, 97%, 96%, 95%, 90%, 85%, or 80% identity to the nucleotide sequence of SEQ ID NO: 19, or 20, and/or (ii) encodes an amino acid sequence comprising the amino acid sequence of SEQ ID NO: 7, or an amino acid sequence having at least 99%, 98%, 97%, 96%, 95%, 90%, 85%, or 80% identity to the amino acid sequence of SEQ ID NO: 7. In one embodiment, RNA administered as described above is nucleoside modified messenger RNA (modRNA) and (i) comprises the nucleotide sequence of SEQ ID NO: 19, or 20; and/or (ii) encodes an amino acid sequence comprising the amino acid sequence of SEQ ID NO: 7.

In one embodiment, RNA administered as described above is nucleoside modified messenger RNA (modRNA) and (i) comprises the nucleotide sequence of SEQ ID NO: 20, a nucleotide sequence having at least 99%, 98%, 97%, 96%, 95%, 90%, 85%, or 80% identity to the nucleotide sequence of SEQ ID NO: 20, and/or (ii) encodes an amino acid sequence comprising the amino acid sequence of SEQ ID NO: 7, or an amino acid sequence having at least 99%, 98%, 97%, 96%, 95%, 90%, 85%, or 80% identity to the amino acid sequence of SEQ ID NO: 7. In one embodiment, RNA administered as described above is nucleoside modified messenger RNA (modRNA) and (i) comprises the nucleotide sequence of SEQ ID NO: 20; and/or (ii) encodes an amino acid sequence comprising the amino acid sequence of SEQ ID NO: 7.

In one embodiment, RNA administered as described above is nucleoside modified messenger RNA (modRNA) and (i) comprises the nucleotide sequence of SEQ ID NO: 30, a nucleotide sequence having at least 99%, 98%, 97%, 96%, 95%, 90%, 85%, or 80% identity to the nucleotide sequence of SEQ ID NO: 30, and/or (ii) encodes an amino acid sequence comprising the amino acid sequence of SEQ ID NO: 29, or an amino acid sequence having at least 99%, 98%, 97%, 96%, 95%, 90%, 85%, or 80% identity to the amino acid sequence of SEQ ID NO: 29. In one embodiment, RNA administered as described above is nucleoside modified messenger RNA (modRNA) and (i) comprises the nucleotide sequence of SEQ ID NO: 30; and/or (ii) encodes an amino acid sequence comprising the amino acid sequence of SEQ ID NO: 29.

In one embodiment, RNA administered is nucleoside modified messenger RNA (modRNA), (i) comprises the nucleotide sequence of SEQ ID NO: 20; and/or (ii) encodes an amino acid sequence comprising the amino acid sequence of SEQ ID NO: 7, and is administered in an amount of about 30 μg per dose. In one embodiment, at least two of such doses are administered. For example, a second dose may be administered about 21 days following administration of the first dose.

In some embodiments, populations to be treated with RNA described herein comprise, essentially consist of, or consist of subjects of age of at least 50, at least 55, at least 60, or at least 65. In some embodiments, populations to be treated with RNA described herein comprise, essentially consist of, or consist of subjects of age of between 55 to 90, 60 to 85, or 65 to 85.

In some embodiments, the period of time between the doses administered is at least 7 days, at least 14 days, or at least 21 days. In some embodiments, the period of time between the doses administered is between 7 days and 28 days such as between 14 days and 23 days.

In some embodiments, no more than 5 doses, no more than 4 doses, or no more than 3 doses of RNA described herein may be administered to a subject.

In some embodiments, the methods and agents described herein are administered (in a regimen, e.g., at a dose, frequency of doses and/or number of doses) such that adverse events (AE), i.e., any unwanted medical occurrence in a patient, e.g., any unfavourable and unintended sign, symptom, or disease associated with the use of a medicinal product, whether or not related to the medicinal product, are mild or moderate in intensity. In some embodiments, the methods and agents described herein are administered such that adverse events (AE) can be managed with interventions such as treatment with, e.g., paracetamol or other drugs that provide analgesic, antipyretic (fever-reducing) and/or anti-inflammatory effects, e.g., nonsteroidal anti-inflammatory drugs (NSAIDs), e.g., aspirin, ibuprofen, and naproxen. Paracetamol or “acetaminophen” which is not classified as a NSAID exerts weak anti-inflammatory effects and can be administered as analgesic according to the present disclosure.

In some embodiments, the methods and agents described herein provide a neutralizing effect in a subject to coronavirus, coronavirus infection, or to a disease or disorder associated with coronavirus.

In some embodiments, the methods and agents described herein following administration to a subject induce an immune response that blocks or neutralizes coronavirus in the subject. In some embodiments, the methods and agents described herein following administration to a subject induce the generation of antibodies such as IgG antibodies that block or neutralize coronavirus in the subject. In some embodiments, the methods and agents described herein following administration to a subject induce an immune response that blocks or neutralizes coronavirus S protein binding to ACE2 in the subject. In some embodiments, the methods and agents described herein following administration to a subject induce the generation of antibodies that block or neutralize coronavirus S protein binding to ACE2 in the subject.

In some embodiments, the methods and agents described herein following administration to a subject induce geometric mean concentrations (GMCs) of RBD domain-binding antibodies such as IgG antibodies of at least 500 U/ml, 1000 U/ml, 2000 U/ml, 3000 U/ml, 4000 U/ml, 5000 U/ml, 10000 U/ml, 15000 U/ml, 20000 U/ml, 25000 U/ml, 30000 U/ml or even higher. In some embodiments, the elevated GMCs of RBD domain-binding antibodies persist for at least 14 days, 21 days, 28 days, 1 month, 3 months, 6 months, 12 months or even longer.

In some embodiments, the methods and agents described herein following administration to a subject induce geometric mean titers (GMTs) of neutralizing antibodies such as IgG antibodies of at least 100 U/ml, 200 U/ml, 300 U/ml, 400 U/ml, 500 U/ml, 1000 U/ml, 1500 U/ml, or even higher. In some embodiments, the elevated GMTs of neutralizing antibodies persist for at least 14 days, 21 days, 28 days, 1 month, 3 months, 6 months, 12 months or even longer.

As used herein, the term “neutralization” refers to an event in which binding agents such as antibodies bind to a biological active site of a virus such as a receptor binding protein, thereby inhibiting the viral infection of cells. As used herein, the term “neutralization” with respect to coronavirus, in particular coronavirus S protein, refers to an event in which binding agents such as antibodies bind to the RBD domain of the S protein, thereby inhibiting the viral infection of cells. In particular, the term “neutralization” refers to an event in which binding agents eliminate or significantly reduce virulence (e.g. ability of infecting cells) of viruses of interest.

The type of immune response generated in response to an antigenic challenge can generally be distinguished by the subset of T helper (Th) cells involved in the response. Immune responses can be broadly divided into two types: Th1 and Th2. Th1 immune activation is optimized for intracellular infections such as viruses, whereas Th2 immune responses are optimized for humoral (antibody) responses. Th1 cells produce interleukin 2 (IL-2), tumor necrosis factor (TNFα) and interferon gamma (IFNγ). Th2 cells produce IL-4, IL-5, IL-6, IL-9, IL-10 and IL-13. Th1 immune activation is the most highly desired in many clinical situations. Vaccine compositions specialized in eliciting Th2 or humoral immune responses are generally not effective against most viral diseases.

In some embodiments, the methods and agents described herein following administration to a subject induce or promote a Th1-mediated immune response in the subject. In some embodiments, the methods and agents described herein following administration to a subject induce or promote a cytokine profile that is typical for a Th1-mediated immune response in the subject. In some embodiments, the methods and agents described herein following administration to a subject induce or promote the production of interleukin 2 (IL-2), tumor necrosis factor (TNFα) and/or interferon gamma (IFNγ) in the subject. In some embodiments, the methods and agents described herein following administration to a subject induce or promote the production of interleukin 2 (IL-2) and interferon gamma (IFNγ) in the subject. In some embodiments, the methods and agents described herein following administration to a subject do not induce or promote a Th2-mediated immune response in the subject, or induce or promote a Th2-mediated immune response in the subject to a significant lower extent compared to the induction or promotion of a Th1-mediated immune response. In some embodiments, the methods and agents described herein following administration to a subject do not induce or promote a cytokine profile that is typical for a Th2-mediated immune response in the subject, or induce or promote a cytokine profile that is typical for a Th2-mediated immune response in the subject to a significant lower extent compared to the induction or promotion of a cytokine profile that is typical for a Th1-mediated immune response. In some embodiments, the methods and agents described herein following administration to a subject do not induce or promote the production of IL-4, IL-5, IL-6, IL-9, IL-10 and/or IL-13, or induce or promote the production of IL-4, IL-5, IL-6, IL-9, IL-10 and/or IL-13 in the subject to a significant lower extent compared to the induction or promotion of interleukin 2 (IL-2), tumor necrosis factor (TNFα) and/or interferon gamma (IFNγ) in the subject. In some embodiments, the methods and agents described herein following administration to a subject do not induce or promote the production of IL-4, or induce or promote the production of IL-4 in the subject to a significant lower extent compared to the induction or promotion of interleukin 2 (IL-2) and interferon gamma (IFNγ) in the subject.

In some embodiments, a composition described herein is characterized in that it produces a seroconversion rate for at least 3 influenza strains that is equal to or greater than that produced by a reference vaccine (e.g., wherein the reference vaccine is a quadrivalent influenza RNA vaccine administered alone, or a commercially approved (non-RNA) influenza vaccine administered in combination with an approved SARS-CoV-2 vaccine).

In some embodiments, a composition described herein is characterized in that it produces influenza neutralizing antibody titers that are within at least two fold of those produced by a reference vaccine for each influenza virus that it encodes antigens of (e.g., wherein the reference vaccine is a quadrivalent influenza RNA vaccine administered alone, or an approved (non-RNA) influenza vaccine administered in combination with an approved SARS-CoV-2 vaccine).

In some embodiments, a composition described herein is characterized in that it produces SARS-CoV-2 neutralizing antibody titers that are within at least two fold of those produced by a reference vaccine (e.g., an approved (non-RNA) influenza vaccine administered in combination with an approved SARS-CoV-2 vaccine).

In some embodiments, a composition described herein is characterized in that it produces local or systemic adverse events at a frequency that is less than 2-fold that of a reference vaccine (e.g., wherein the reference vaccine is a quadrivalent influenza RNA vaccine administered alone, or a commercial (non-RNA) vaccine administered in combination with a SARS-CoV-2 RNA vaccine).

In some embodiments, the methods and agents described herein following administration to a subject induce an antibody response, in particular a neutralizing antibody response, in the subject that targets a panel of different S protein variants such as SARS-CoV-2 S protein variants, in particular naturally occurring S protein variants. In some embodiments, the panel of different S protein variants comprises at least 5, at least 10, at least 15, or even more S protein variants. In some embodiments, such S protein variants comprise variants having amino acid modifications in the RBD domain and/or variants having amino acid modifications outside the RBD domain. In one embodiment, such S protein variant comprises SARS-CoV-2 S protein or a naturally occurring variant thereof wherein the amino acid corresponding to position 321 (Q) in SEQ ID NO: 1 is S. In one embodiment, such S protein variant comprises SARS-CoV-2 S protein or a naturally occurring variant thereof wherein the amino acid corresponding to position 321 (Q) in SEQ ID NO: 1 is L. In one embodiment, such S protein variant comprises SARS-CoV-2 S protein or a naturally occurring variant thereof wherein the amino acid corresponding to position 341 (V) in SEQ ID NO: 1 is I. In one embodiment, such S protein variant comprises SARS-CoV-2 S protein or a naturally occurring variant thereof wherein the amino acid corresponding to position 348 (A) in SEQ ID NO: 1 is T. In one embodiment, such S protein variant comprises SARS-CoV-2 S protein or a naturally occurring variant thereof wherein the amino acid corresponding to position 354 (N) in SEQ ID NO: 1 is D. In one embodiment, such S protein variant comprises SARS-CoV-2 S protein or a naturally occurring variant thereof wherein the amino acid corresponding to position 359 (S) in SEQ ID NO: 1 is N. In one embodiment, such S protein variant comprises SARS-CoV-2 S protein or a naturally occurring variant thereof wherein the amino acid corresponding to position 367 (V) in SEQ ID NO: 1 is F. In one embodiment, such S protein variant comprises SARS-CoV-2 S protein or a naturally occurring variant thereof wherein the amino acid corresponding to position 378 (K) in SEQ ID NO: 1 is S. In one embodiment, such S protein variant comprises SARS-CoV-2 S protein or a naturally occurring variant thereof wherein the amino acid corresponding to position 378 (K) in SEQ ID NO: 1 is R. In one embodiment, such S protein variant comprises SARS-CoV-2 S protein or a naturally occurring variant thereof wherein the amino acid corresponding to position 408 (R) in SEQ ID NO: 1 is I. In one embodiment, such S protein variant comprises SARS-CoV-2 S protein or a naturally occurring variant thereof wherein the amino acid corresponding to position 409 (Q) in SEQ ID NO: 1 is E. In one embodiment, such S protein variant comprises SARS-CoV-2 S protein or a naturally occurring variant thereof wherein the amino acid corresponding to position 435 (A) in SEQ ID NO: 1 is S. In one embodiment, such S protein variant comprises SARS-CoV-2 S protein or a naturally occurring variant thereof wherein the amino acid corresponding to position 439 (N) in SEQ ID NO: 1 is K. In one embodiment, such S protein variant comprises SARS-CoV-2 S protein or a naturally occurring variant thereof wherein the amino acid corresponding to position 458 (K) in SEQ ID NO: 1 is R. In one embodiment, such S protein variant comprises SARS-CoV-2 S protein or a naturally occurring variant thereof wherein the amino acid corresponding to position 472 (I) in SEQ ID NO: 1 is V. In one embodiment, such S protein variant comprises SARS-CoV-2 S protein or a naturally occurring variant thereof wherein the amino acid corresponding to position 476 (G) in SEQ ID NO: 1 is S. In one embodiment, such S protein variant comprises SARS-CoV-2 S protein or a naturally occurring variant thereof wherein the amino acid corresponding to position 477 (S) in SEQ ID NO: 1 is N. In one embodiment, such S protein variant comprises SARS-CoV-2 S protein or a naturally occurring variant thereof wherein the amino acid corresponding to position 483 (V) in SEQ ID NO: 1 is A. In one embodiment, such S protein variant comprises SARS-CoV-2 S protein or a naturally occurring variant thereof wherein the amino acid corresponding to position 508 (Y) in SEQ ID NO: 1 is H. In one embodiment, such S protein variant comprises SARS-CoV-2 S protein or a naturally occurring variant thereof wherein the amino acid corresponding to position 519 (H) in SEQ ID NO: 1 is P. In one embodiment, such S protein variant comprises SARS-CoV-2 S protein or a naturally occurring variant thereof wherein the amino acid corresponding to position 614 (D) in SEQ ID NO: 1 is G.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a mutation at a position corresponding to position 501 (N) in SEQ ID NO: 1. In one embodiment, the amino acid corresponding to position 501 (N) in SEQ ID NO: 1 is Y.

Said S protein variant comprising a mutation at a position corresponding to position 501 (N) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 614 (D), 681 (P), 716 (T), 982 (S), 1118 (D), 80 (D), 215 (D), 484 (E), 701 (A), 18 (L), 246 (R), 417 (K), 242 (L), 243 (A), and 244 (L). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted.

In some embodiments, the methods and agents described herein following administration to a subject induce an antibody response, in particular a neutralizing antibody response, in the subject that targets VOC-202012/01.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: deletion 69-70, deletion 144, N501Y, A570D, D614G, P681H, T716I, S982A, and D1118H.

In some embodiments, the methods and agents described herein following administration to a subject induce an antibody response, in particular a neutralizing antibody response, in the subject that targets 501.V2.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y and A701V, and optionally: L18F, R246I, K417N, and deletion 242-244. Said S protein variant may also comprise a D->G mutation at a position corresponding to position 614 in SEQ ID NO: 1.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a deletion at a position corresponding to positions 69 (H) and 70 (V) in SEQ ID NO: 1.

In some embodiments, a S protein variant comprising a deletion at a position corresponding to positions 69 (H) and 70 (V) in SEQ ID NO: 1 may comprise one or more further mutations. Such one or more further mutations may be selected from mutations at positions corresponding to the following positions in SEQ ID NO: 1: 144 (Y), 501 (N), 570 (A), 614 (D), 681 (P), 716 (T), 982 (S), 1118 (D), 80 (D), 215 (D), 484 (E), 701 (A), 18 (L), 246 (R), 417 (K), 242 (L), 243 (A), 244 (L), 453 (Y), 692 (I), 1147 (S), and 1229 (M). In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A.

In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I.

In some embodiments, the methods and agents described herein following administration to a subject induce an antibody response, in particular a neutralizing antibody response, in the subject that targets VOC-202012/01.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: deletion 69-70, deletion 144, N501Y, A570D, D614G, P681H, T716I, S982A, and D1118H.

In some embodiments, the methods and agents described herein following administration to a subject induce an antibody response, in particular a neutralizing antibody response, in the subject that targets “Cluster 5”.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: deletion 69-70, Y453F, I692V, M1229I, and optionally S1147L.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a mutation at a position corresponding to position 614 (D) in SEQ ID NO: 1. In one embodiment, the amino acid corresponding to position 614 (D) in SEQ ID NO: 1 is G.

In some embodiments, a S protein variant comprising a mutation at a position corresponding to position 614 (D) in SEQ ID NO: 1 may comprise one or more further mutations. Such one or more further mutations may be selected from mutations at positions corresponding to the following positions in SEQ ID NO: 1: 69 (H), 70 (V), 144 (Y), 501 (N), 570 (A), 681 (P), 716 (T), 982 (S), 1118 (D), 80 (D), 215 (D), 484 (E), 701 (A), 18 (L), 246 (R), 417 (K), 242 (L), 243 (A), 244 (L), 453 (Y), 692 (I), 1147 (S), and 1229 (M). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I.

In some embodiments, the methods and agents described herein following administration to a subject induce an antibody response, in particular a neutralizing antibody response, in the subject that targets VOC-202012/01.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: deletion 69-70, deletion 144, N501Y, A570D, D614G, P681H, T716I, S982A, and D1118H.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, D614G and A701V, and optionally: L18F, R246I, K417N, and deletion 242-244.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a mutation at positions corresponding to positions 501 (N), 484 (E), and 614 (D) in SEQ ID NO: 1. In one embodiment, the amino acid corresponding to position 501 (N) in SEQ ID NO: 1 is Y, the amino acid corresponding to position 484 (E) in SEQ ID NO: 1 is K, and the amino acid corresponding to position 614 (D) in SEQ ID NO: 1 is G.

In some embodiments, a S protein variant comprising a mutation at positions corresponding to positions 501 (N) and 614 (D) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 681 (P), 716 (T), 982 (S), 1118 (D), 80 (D), 215 (D), 484 (E), 701 (A), 18 (L), 246 (R), 417 (K), 242 (L), 243 (A), 244 (L), 453 (Y), 692 (I), 1147 (S), and 1229 (M). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I.

In some embodiments, the methods and agents described herein following administration to a subject induce an antibody response, in particular a neutralizing antibody response, in the subject that targets VOC-202012/01.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: deletion 69-70, deletion 144, N501Y, A570D, D614G, P681H, T716I, S982A, and D1118H.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, D614G and A701V, and optionally: L18F, R246I, K417N, and deletion 242-244.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a mutation at a position corresponding to position 484 (E) in SEQ ID NO: 1. In one embodiment, the amino acid corresponding to position 484 (E) in SEQ ID NO: 1 is K.

In some embodiments, a S protein variant comprising a mutation at a position corresponding to position 484 (E) in SEQ ID NO: 1 may comprise one or more further mutations. Such one or more further mutations may be selected from mutations at positions corresponding to the following positions in SEQ ID NO: 1: 69 (H), 70 (V), 144 (Y), 501 (N), 570 (A), 614 (D), 681 (P), 716 (T), 982 (S), 1118 (D), 80 (D), 215 (D), 701 (A), 18 (L), 246 (R), 417 (K), 242 (L), 243 (A), 244 (L), 453 (Y), 692 (I), 1147 (S), 1229 (M), 20 (T), 26 (P), 138 (D), 190 (R), 417 (K), 655 (H), 1027 (T), and 1176 (V). In one embodiment, the amino acid corresponding to position 69 (H) in SEQ ID NO: 1 is deleted.

In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 417 (K) in SEQ ID NO: 1 is T. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the methods and agents described herein following administration to a subject induce an antibody response, in particular a neutralizing antibody response, in the subject that targets 501.V2.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y and A701V, and optionally: L18F, R246I, K417N, and deletion 242-244. Said S protein variant may also comprise a D->G mutation at a position corresponding to position 614 in SEQ ID NO: 1.

In some embodiments, the methods and agents described herein following administration to a subject induce an antibody response, in particular a neutralizing antibody response, in the subject that targets “B.1.1.28”.

In some embodiments, the methods and agents described herein following administration to a subject induce an antibody response, in particular a neutralizing antibody response, in the subject that targets “B.1.1.248”.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: L18F, T20N, P26S, D138Y, R190S, K417T, E484K, N501Y, H655Y, T1027I, and V1176F.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a mutation at positions corresponding to positions 501 (N) and 484 (E) in SEQ ID NO: 1. In one embodiment, the amino acid corresponding to position 501 (N) in SEQ ID NO: 1 is Y and the amino acid corresponding to position 484 (E) in SEQ ID NO: 1 is K.

In some embodiments, a S protein variant comprising a mutation at positions corresponding to positions 501 (N) and 484 (E) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 614 (D), 681 (P), 716 (T), 982 (S), 1118 (D), 80 (D), 215 (D), 701 (A), 18 (L), 246 (R), 417 (K), 242 (L), 243 (A), 244 (L), 453 (Y), 692 (I), 1147 (S), 1229 (M), 20 (T), 26 (P), 138 (D), 190 (R), 417 (K), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 417 (K) in SEQ ID NO: 1 is T. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the methods and agents described herein following administration to a subject induce an antibody response, in particular a neutralizing antibody response, in the subject that targets 501.V2.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y and A701V, and optionally: L18F, R246I, K417N, and deletion 242-244. Said S protein variant may also comprise a D->G mutation at a position corresponding to position 614 in SEQ ID NO: 1.

In some embodiments, the methods and agents described herein following administration to a subject induce an antibody response, in particular a neutralizing antibody response, in the subject that targets “B.1.1.248”.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: L18F, T20N, P26S, D138Y, R190S, K417T, E484K, N501Y, H655Y, T1027I, and V1176F.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a mutation at positions corresponding to positions 501 (N), 484 (E) and 614 (D) in SEQ ID NO: 1. In one embodiment, the amino acid corresponding to position 501 (N) in SEQ ID NO: 1 is Y, the amino acid corresponding to position 484 (E) in SEQ ID NO: 1 is K and the amino acid corresponding to position 614 (D) in SEQ ID NO: 1 is G.

In some embodiments, a S protein variant comprising a mutation at positions corresponding to positions 501 (N), 484 (E) and 614 (D) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 681 (P), 716 (T), 982 (S), 1118 (D), 80 (D), 215 (D), 701 (A), 18 (L), 246 (R), 417 (K), 242 (L), 243 (A), 244 (L), 453 (Y), 692 (I), 1147 (S), 1229 (M), 20 (T), 26 (P), 138 (D), 190 (R), 417 (K), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted.

In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 417 (K) in SEQ ID NO: 1 is T. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, A701V, and D614G, and optionally: L18F, R246I, K417N, and deletion 242-244.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a deletion at a position corresponding to positions 242 (L), 243 (A) and 244 (L) in SEQ ID NO: 1.

In some embodiments, a S protein variant comprising a deletion at a position corresponding to positions 242 (L), 243 (A) and 244 (L) in SEQ ID NO: 1 may comprise one or more further mutations. Such one or more further mutations may be selected from mutations at positions corresponding to the following positions in SEQ ID NO: 1: 69 (H), 70 (V), 144 (Y), 501 (N), 570 (A), 614 (D), 681 (P), 716 (T), 982 (S), 1118 (D), 80 (D), 215 (D), 484 (E), 701 (A), 18 (L), 246 (R), 417 (K), 453 (Y), 692 (I), 1147 (S), 1229 (M), 20 (T), 26 (P), 138 (D), 190 (R), 417 (K), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted.

In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 417 (K) in SEQ ID NO: 1 is N. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 417 (K) in SEQ ID NO: 1 is T. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the methods and agents described herein following administration to a subject induce an antibody response, in particular a neutralizing antibody response, in the subject that targets 501.V2.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, A701V and deletion 242-244, and optionally: L18F, R246I, and K417N. Said S protein variant may also comprise a D->G mutation at a position corresponding to position 614 in SEQ ID NO: 1.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a mutation at a position corresponding to position 417 (K) in SEQ ID NO: 1. In one embodiment, the amino acid corresponding to position 417 (K) in SEQ ID NO: 1 is N. In one embodiment, the amino acid corresponding to position 417 (K) in SEQ ID NO: 1 is T.

In some embodiments, a S protein variant comprising a mutation at a position corresponding to position 417 (K) in SEQ ID NO: 1 may comprise one or more further mutations. Such one or more further mutations may be selected from mutations at positions corresponding to the following positions in SEQ ID NO: 1: 69 (H), 70 (V), 144 (Y), 501 (N), 570 (A), 614 (D), 681 (P), 716 (T), 982 (S), 1118 (D), 80 (D), 215 (D), 484 (E), 701 (A), 18 (L), 246 (R), 242 (L), 243 (A), 244 (L), 453 (Y), 692 (I), 1147 (S), 1229 (M), 20 (T), 26 (P), 138 (D), 190 (R), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 501 (N) in SEQ ID NO: 1 is Y. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 484 (E) in SEQ ID NO: 1 is K. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the methods and agents described herein following administration to a subject induce an antibody response, in particular a neutralizing antibody response, in the subject that targets 501.V2.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, A701V, and K417N, and optionally: L18F, R246I, and deletion 242-244. Said S protein variant may also comprise a D->G mutation at a position corresponding to position 614 in SEQ ID NO: 1.

In some embodiments, the methods and agents described herein following administration to a subject induce an antibody response, in particular a neutralizing antibody response, in the subject that targets “B.1.1.248”.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: L18F, T20N, P26S, D138Y, R190S, K417T, E484K, N501Y, H655Y, T1027I, and V1176F.

In some embodiments, the methods and agents described herein following administration to a subject induce an antibody response, in particular a neutralizing antibody response, in the subject that targets a S protein variant such as SARS-CoV-2 S protein variant, in particular naturally occurring S protein variant comprising a mutation at positions corresponding to positions 417 (K) and 484 (E) and/or 501 (N) in SEQ ID NO: 1. In one embodiment, the amino acid corresponding to position 417 (K) in SEQ ID NO: 1 is N, and the amino acid corresponding to position 484 (E) in SEQ ID NO: 1 is K and/or the amino acid corresponding to position 501 (N) in SEQ ID NO: 1 is Y. In one embodiment, the amino acid corresponding to position 417 (K) in SEQ ID NO: 1 is T, and the amino acid corresponding to position 484 (E) in SEQ ID NO: 1 is K and/or the amino acid corresponding to position 501 (N) in SEQ ID NO: 1 is Y.

In some embodiments, a S protein variant comprising a mutation at positions corresponding to positions 417 (K) and 484 (E) and/or 501 (N) in SEQ ID NO: 1 may comprise one or more further mutations. Such one or more further mutations may be selected from mutations at positions corresponding to the following positions in SEQ ID NO: 1: 69 (H), 70 (V), 144 (Y), 570 (A), 614 (D), 681 (P), 716 (T), 982 (S), 1118 (D), 80 (D), 215 (D), 701 (A), 18 (L), 246 (R), 242 (L), 243 (A), 244 (L), 453 (Y), 692 (I), 1147 (S), 1229 (M), 20 (T), 26 (P), 138 (D), 190 (R), 655 (H), 1027 (T), and 1176 (V). In one embodiment, the amino acid corresponding to position 69 (H) in SEQ ID NO: 1 is deleted. In one embodiment, the amino acid corresponding to position 70 (V) in SEQ ID NO: 1 is deleted. In one embodiment, the amino acid corresponding to position 144 (Y) in SEQ ID NO: 1 is deleted. In one embodiment, the amino acid corresponding to position 570 (A) in SEQ ID NO: 1 is D. In one embodiment, the amino acid corresponding to position 614 (D) in SEQ ID NO: 1 is G. In one embodiment, the amino acid corresponding to position 681 (P) in SEQ ID NO: 1 is H. In one embodiment, the amino acid corresponding to position 716 (T) in SEQ ID NO: 1 is I. In one embodiment, the amino acid corresponding to position 982 (S) in SEQ ID NO: 1 is A. In one embodiment, the amino acid corresponding to position 1118 (D) in SEQ ID NO: 1 is H. In one embodiment, the amino acid corresponding to position 80 (D) in SEQ ID NO: 1 is A. In one embodiment, the amino acid corresponding to position 215 (D) in SEQ ID NO: 1 is G. In one embodiment, the amino acid corresponding to position 701 (A) in SEQ ID NO: 1 is V. In one embodiment, the amino acid corresponding to position 18 (L) in SEQ ID NO: 1 is F. In one embodiment, the amino acid corresponding to position 246 (R) in SEQ ID NO: 1 is I. In one embodiment, the amino acid corresponding to position 242 (L) in SEQ ID NO: 1 is deleted. In one embodiment, the amino acid corresponding to position 243 (A) in SEQ ID NO: 1 is deleted. In one embodiment, the amino acid corresponding to position 244 (L) in SEQ ID NO: 1 is deleted. In one embodiment, the amino acid corresponding to position 453 (Y) in SEQ ID NO: 1 is F. In one embodiment, the amino acid corresponding to position 692 (I) in SEQ ID NO: 1 is V. In one embodiment, the amino acid corresponding to position 1147 (S) in SEQ ID NO: 1 is L. In one embodiment, the amino acid corresponding to position 1229 (M) in SEQ ID NO: 1 is I. In one embodiment, the amino acid corresponding to position 20 (T) in SEQ ID NO: 1 is N. In one embodiment, the amino acid corresponding to position 26 (P) in SEQ ID NO: 1 is S. In one embodiment, the amino acid corresponding to position 138 (D) in SEQ ID NO: 1 is Y. In one embodiment, the amino acid corresponding to position 190 (R) in SEQ ID NO: 1 is S. In one embodiment, the amino acid corresponding to position 655 (H) in SEQ ID NO: 1 is Y. In one embodiment, the amino acid corresponding to position 1027 (T) in SEQ ID NO: 1 is I. In one embodiment, the amino acid corresponding to position 1176 (V) in SEQ ID NO: 1 is F.

In some embodiments, the methods and agents described herein following administration to a subject induce an antibody response, in particular a neutralizing antibody response, in the subject that targets 501.V2.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: D80A, D215G, E484K, N501Y, A701V, and K417N and optionally: L18F, R246I, and deletion 242-244. Said S protein variant may also comprise a D->G mutation at a position corresponding to position 614 in SEQ ID NO: 1.

In some embodiments, the methods and agents described herein following administration to a subject induce an antibody response, in particular a neutralizing antibody response, in the subject that targets “B.1.1.248”.

In some embodiments, the methods and agents described herein following administration to a subject induce an antibody response, in particular a neutralizing antibody response, in the subject that targets a S protein variant comprising the following mutations at positions corresponding to the following positions in SEQ ID NO: 1: L18F, T20N, P26S, D138Y, R190S, K417T, E484K, N501Y, H655Y, T1027I, and V1176F.

In some embodiments, the methods and agents described herein following administration to a subject induce an antibody response, in particular a neutralizing antibody response, in the subject that targets the Omicron (B.1.1.529) variant.

In some embodiments, the methods and agents described herein following administration to a subject induce an antibody response, in particular a neutralizing antibody response, in the subject that targets a S protein variant comprising at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37 of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, N501Y, S375F, Y505H, V143del, H69del, V70del, N211del, L212I, ins214EPE, G142D, Y144del, Y145del, L141del, Y144F, Y145D, G142del, as compared to SEQ ID NO: 1.

In some embodiments, the methods and agents described herein following administration to a subject induce an antibody response, in particular a neutralizing antibody response, in the subject that targets a S protein variant comprising at least 10, at least 15, at least 20, at least 21, at least 22, at least 23, at least 24, or all of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, as compared to SEQ ID NO: 1. Said S protein variant may include at least 1, at least 2, at least 3, at least 4, at least 5, or all of the following mutations: N501Y, S375F, Y505H, V143del, H69del, V70del, as compared to SEQ ID NO: 1 and/or may include at least 1, at least 2, at least 3, at least 4, at least 5, or all of the following mutations: N211del, L212I, ins214EPE, G142D, Y144del, Y145del, as compared to SEQ ID NO: 1. In some embodiments, said S protein variant may include at least 1, at least 2, at least 3, or all of the following mutations: L141del, Y144F, Y145D, G142del, as compared to SEQ ID NO: 1.

In some embodiments, the methods and agents described herein following administration to a subject induce an antibody response, in particular a neutralizing antibody response, in the subject that targets a S protein variant comprising at least 10, at least 15, at least 20, at least 21, at least 22, at least 23, at least 24, at least 25, at least 26, at least 27, at least 28, at least 29, at least 30, at least 31, at least 32, or at least 33 of the following mutations: A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the methods and agents described herein following administration to a subject induce an antibody response, in particular a neutralizing antibody response, in the subject that targets a S protein variant comprising the following mutations:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the methods and agents described herein following administration to a subject induce an antibody response, in particular a neutralizing antibody response, in the subject that targets a S protein variant comprising the following mutations:
A67V, Δ69-70, T95I, G142D, Δ143-145, Δ211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the methods and agents described herein following administration to a subject induce an immune response (cellular and/or antibody response, in particular neutralizing antibody response) in the subject that targets the Omicron (B.1.1.529) variant.

In some embodiments, the methods and agents described herein following administration to a subject induce an immune response (cellular and/or antibody response, in particular neutralizing antibody response) in the subject that targets a S protein variant comprising at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37 of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, N501Y, S375F, Y505H, V143del, H69del, V70del, N211del, L212I, ins214EPE, G142D, Y144del, Y145del, L141del, Y144F, Y145D, G142del, as compared to SEQ ID NO: 1.

In some embodiments, the methods and agents described herein following administration to a subject induce an immune response (cellular and/or antibody response, in particular neutralizing antibody response) in the subject that targets a S protein variant comprising at least 10, at least 15, at least 20, at least 21, at least 22, at least 23, at least 24, or all of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, as compared to SEQ ID NO: 1. Said S protein variant may include at least 1, at least 2, at least 3, at least 4, at least 5, or all of the following mutations: N501Y, S375F, Y505H, V143del, H69del, V70del, as compared to SEQ ID NO: 1 and/or may include at least 1, at least 2, at least 3, at least 4, at least 5, or all of the following mutations: N211del, L212I, ins214EPE, G142D, Y144del, Y145del, as compared to SEQ ID NO: 1. In some embodiments, said S protein variant may include at least 1, at least 2, at least 3, or all of the following mutations: L141del, Y144F, Y145D, G142del, as compared to SEQ ID NO: 1.

In some embodiments, the methods and agents described herein following administration to a subject induce an immune response (cellular and/or antibody response, in particular neutralizing antibody response) in the subject that targets a S protein variant comprising at least 10, at least 15, at least 20, at least 21, at least 22, at least 23, at least 24, at least 25, at least 26, at least 27, at least 28, at least 29, at least 30, at least 31, at least 32, or at least 33 of the following mutations:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the methods and agents described herein following administration to a subject induce an immune response (cellular and/or antibody response, in particular neutralizing antibody response) in the subject that targets a S protein variant comprising the following mutations:
A67V, 469-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, the methods and agents described herein following administration to a subject induce an immune response (cellular and/or antibody response, in particular neutralizing antibody response) in the subject that targets a S protein variant comprising the following mutations:
A67V, Δ69-70, T95I, G142D, Δ143-145, Δ211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, methods and agents described herein upon administration to a subject induce an antibody response, in particular a neutralizing antibody response, in the subject that targets one or more S protein variants comprising the mutations associated with a given Omicron variant listed in Table 2 (e.g., (i) an XBB.1.5 variant, (ii) an XBB.1.5, XBB.1.16, and XBB.2.3 variant, and/or (iii) one or more XBB variants (e.g., each of the XBB variants listed in Table 2).

The term “amino acid corresponding to position . . . ” as used herein refers to an amino acid position number corresponding to an amino acid position number in SARS-CoV-2 S protein, in particular the amino acid sequence shown in SEQ ID NO: 1. The phrase “as compared to SEQ ID NO: 1” is equivalent to “at positions corresponding to the following positions in SEQ ID NO: 1”. Corresponding amino acid positions in other coronavirus S protein variants such as SARS-CoV-2 S protein variants may be found by alignment with SARS-CoV-2 S protein, in particular the amino acid sequence shown in SEQ ID NO: 1. It is considered well-known in the art how to align a sequence or segment in a sequence and thereby determine the corresponding position in a sequence to an amino acid position according to the present disclosure. Standard sequence alignment programs such as ALIGN, ClustalW or similar, typically at default settings may be used.

In some embodiments, the panel of different S protein variants to which an antibody response is targeted comprises at least 5, at least 10, at least 15, or even more S protein variants selected from the group consisting of the Q321S, V341I, A348T, N354D, S359N, V367F, K378S, R408I, Q409E, A435S, K458R, I472V, G476S, V483A, Y508H, H519P and D614G variants described above. In some embodiments, the panel of different S protein variants to which an antibody response is targeted comprises all S protein variants from the group consisting of the Q321S, V341I, A348T, N354D, S359N, V367F, K378S, R408I, Q409E, A435S, K458R, 1472V, G476S, V483A, Y508H, H519P and D614G variants described above.

In some embodiments, the panel of different S protein variants to which an antibody response is targeted comprises at least 5, at least 10, at least 15, or even more S protein variants selected from the group consisting of the Q321L, V341I, A348T, N354D, S359N, V367F, K378R, R408I, Q409E, A435S, N439K, K458R, 1472V, G476S, S477N, V483A, Y508H, H519P and D614G variants described above. In some embodiments, the panel of different S protein variants to which an antibody response is targeted comprises all S protein variants from the group consisting of the Q321L, V341I, A348T, N354D, S359N, V367F, K378R, R408I, Q409E, A435S, N439K, K458R, 1472V, G476S, S477N, V483A, Y508H, H519P and D614G variants described above.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises one or more of the mutations described herein for S protein variants such as SARS-CoV-2 S protein variants, in particular naturally occurring S protein variants. In one embodiment, a SARS-CoV-2 S protein, an immunogenic variant thereof, or an immunogenic fragment of the SARS-CoV-2 S protein or the immunogenic variant thereof, e.g., as encoded by RNA described herein, comprises a mutation at a position corresponding to position 501 (N) in SEQ ID NO: 1. In one embodiment, the amino acid corresponding to position 501 (N) in SEQ ID NO: 1 is Y. In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises one or more mutations, such as all mutations, of a SARS-CoV-2 S protein of a SARS-CoV-2 variant selected from the group consisting of VOC-202012/01, 501.V2, Cluster 5 and B.1.1.248. In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises an amino acid sequence with alanine substitution at position 80, glycine substitution at position 215, lysine substitution at position 484, tyrosine substitution at position 501, valine substitution at position 701, phenylalanine substitution at position 18, isoleucine substitution at position 246, asparagine substitution at position 417, glycine substitution at position 614, deletions at positions 242 to 244, and proline substitutions at positions 986 and 987 of SEQ ID NO:1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, or at least 37 of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, N501Y, S375F, Y505H, V143del, H69del, V70del, N211del, L212I, ins214EPE, G142D, Y144del, Y145del, L141del, Y144F, Y145D, G142del, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises at least 10, at least 15, at least 20, at least 21, at least 22, at least 23, at least 24, or all of the following mutations: T547K, H655Y, D614G, N679K, P681H, N969K, S373P, S371L, N440K, G339D, G446S, N856K, N764K, K417N, D796Y, Q954H, T95I, A67V, L981F, S477N, G496S, T478K, Q498R, Q493R, E484A, as compared to SEQ ID NO: 1. Said SARs-COV-2 S protein, variant, or fragment may include at least 1, at least 2, at least 3, at least 4, at least 5, or all of the following mutations: N501Y, S375F, Y505H, V143del, H69del, V70del, as compared to SEQ ID NO: 1 and/or may include at least 1, at least 2, at least 3, at least 4, at least 5, or all of the following mutations: N211del, L212I, ins214EPE, G142D, Y144del, Y145del, as compared to SEQ ID NO: 1. In some embodiments, said SARs-CoV-2 S protein, variant, or fragment may include at least 1, at least 2, at least 3, or all of the following mutations: L141del, Y144F, Y145D, G142del, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises at least 10, at least 15, at least 20, at least 21, at least 22, at least 23, at least 24, at least 25, at least 26, at least 27, at least 28, at least 29, at least 30, at least 31, at least 32, or at least 33 of the following mutations:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1. In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises the following mutations:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises the following mutations:
A67V, 469-70, T95I, G142D, Δ143-145, Δ211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, and L981F, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises the following mutations:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises the following mutations:
A67V, Δ69-70, T95I, G142D, Δ143-145, Δ211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises the following mutations:

In some embodiments, the spike changes in Omicron BA.2 variant include T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, K986P, and V987P, as compared to SEQ ID NO: 1.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprises the following mutations:
T19I, Δ24-26, A27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, K986P, and V987P, as compared to SEQ ID NO: 1. In some embodiments, administration of a variant specific vaccine (e.g., a variant specific vaccine disclosed herein) may result in an improved immune response in a patient as compared to administration of vaccine encoding or comprising a SARS-CoV-2 S protein from a Wuhan strain, or an immunogenic fragment thereof. In some embodiments, administration of a variant-specific vaccine may result in induction of a broader immune response in a subject as compared to a patient administered a vaccine comprising or encoding a SARS-CoV-2 S protein from a Wuhan strain (or an immunogenic fragment thereof) (e.g., induce a stronger neutralization response against a greater number of SARS-CoV-2 variants and/or a neutralization response that recognizes epitopes in a greater number of SARS-CoV-2 variants). In particular embodiments, a broader immune response may be induced when a variant specific vaccine is administered in combination with a vaccine comprising or encoding a SARS-CoV-2 S protein from a different variant or from a Wuhan strain (e.g., in some embodiments, a broader immune response may be induced when a variant specific vaccine is administered in combination with a vaccine comprising or encoding a SARS-CoV-2 S protein from a Wuhan strain or a vaccine comprising or encoding a SARS-CoV-2 S protein comprising mutations characteristic of a different SARS-CoV-2 variant). For example, a broader immune response may be induced when an RNA vaccine encoding a SARS-CoV-2 S protein from a Wuhan strain is administered in combination with an RNA vaccine encoding a SARS-CoV-2 S protein having mutations characteristic of an Omicron variant (e.g., a BA.1, BA.4/5, or XBB.1.5 Omicron variant). In another embodiment, a broader immune response may be induced when an RNA vaccine encoding a SARS-CoV-2 S protein comprising one or more mutations characteristic of a delta variant is administered in combination with an RNA vaccine encoding a SARS-CoV-2 S protein comprising one or more mutations characteristic of an Omicron variant (e.g., a BA.1, BA.4/5, or XBB.1.5 Omicron variant). In such embodiments, a “broader” immune response may be defined relative to a patient administered a vaccine comprising or encoding a SARS-CoV-2 S protein from a single variant (e.g., an RNA vaccine encoding a SARS-CoV-2 S protein from a Wuhan strain). Vaccines comprising or encoding S proteins from different SARS-CoV-2 variants, or immunogenic fragments thereof, may be administered in combination by administering at different time points (e.g., administering a vaccine encoding a SARS-CoV-2 S protein from a Wuhan strain and a vaccine encoding a SARS-CoV-2 S protein having one or more mutations characteristic of a variant strain at different time points, e.g., both administered as part of a primary regimen or part of a booster regimen; or one is administered as part of a primary regimen while another is administered as part of a booster regimen). In some embodiments, vaccines comprising or encoding S proteins from different SARS-CoV-2 variants, or immunogenic fragments thereof, may be administered in combination by administering a multivalent vaccine (e.g., a composition comprising RNA encoding a SARS-CoV-2 S protein from a Wuhan strain and RNA encoding a SARS-CoV-2 S protein having mutations characteristic of an Omicron variant (e.g., a BA.1, BA.4/5, or XBB.1.5 Omicron variant)). In some embodiments, a variant specific vaccine may induce a superior immune response (e.g., inducing higher concentrations of neutralizing antibodies) against a variant against which the vaccine is specifically designed to immunize, and an immune response against one or more other variants. In some such embodiments, an immune response against other variant(s) may be comparable to or higher than that as observed with a vaccine that encodes or comprises a SARS-CoV-2 S protein from a Wuhan strain.

In some embodiments, the geometric mean ratio (GMR) or geometric mean fold rise (GMFR) of neutralization antibodies induced by a variant specific vaccine is at least 1.1, 1.2, 1.3, 1.4, 1.5, 1.6, 1.7, 1.8, 1.9, or 2.0 (e.g., 1.1 to 4, 1.1 to 3.5, 1.1 to 3, 1.5 to 3, or 1.1 to 1.5) fold higher than that induced by a non-variant specific vaccine (e.g., as measured 1 day to 3 months after immunization, 7 days to 2 months after administration, about 7 days, or about 1 month after administration).

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises the amino acid sequence of SEQ ID NO: 49, an amino acid sequence having at least 99.5%, 99%, 98.5%, 98%, 98.5%, 97%, 96%, 95%, 90%, 85%, or 80% identity to the amino acid sequence of SEQ ID NO: 49, or an immunogenic fragment of the amino acid sequence of SEQ ID NO: 49, or the amino acid sequence having at least 99.5%, 99%, 98.5%, 98%, 98.5%, 97%, 96%, 95%, 90%, 85%, or 80% identity to the amino acid sequence of SEQ ID NO: 49. In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises the amino acid sequence of SEQ ID NO: 49.

In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises the amino acid sequence of SEQ ID NO: 52, an amino acid sequence having at least 99.5%, 99%, 98.5%, 98%, 98.5%, 97%, 96%, 95%, 90%, 85%, or 80% identity to the amino acid sequence of SEQ ID NO: 52, or an immunogenic fragment of the amino acid sequence of SEQ ID NO: 52, or the amino acid sequence having at least 99.5%, 99%, 98.5%, 98%, 98.5%, 97%, 96%, 95%, 90%, 85%, or 80% identity to the amino acid sequence of SEQ ID NO: 52. In some embodiments, a SARS-CoV-2 S protein, an immunogenic variant thereof, or an immunogenic fragment of the SARS-CoV-2 S protein or the immunogenic variant thereof, e.g., as encoded by RNA described herein, comprising said mutations comprises the amino acid sequence of SEQ ID NO: 52.

In some embodiments, the methods and agents, e.g., mRNA compositions, described herein following administration to a subject induce a cell-mediated immune response (e.g., CD4+ and/or CD8+ T cell response). In some embodiments, T cells are induced that recognize one or more epitopes (e.g., MHC class I-restricted epitopes) selected from the group consisting of LPFNDGVYF (SEQ ID NO: 42), GVYFASTEK (SEQ ID NO: 43), YLOPRTFLL (SEQ ID NO: 35), QPTESIVRF (SEQ ID NO: 40), CVADYSVLY (SEQ ID NO: 44), KCYGVSPTK (SEQ ID NO: 45), NYNYLYRLF (SEQ ID NO: 38), FQPTNGVGY (SEQ ID NO: 46), IPFAMQMAY (SEQ ID NO: 41), RLQSLQTYV (SEQ ID NO: 36), GTHWFVTQR (SEQ ID NO: 47), VYDPLQPEL (SEQ ID NO: 48), QYIKWPWYI (SEQ ID NO: 37), and KWPWYIWLGF (SEQ ID NO: 39). In one embodiment, T cells are induced that recognize the epitope YLQPRTFLL (SEQ ID NO: 35). In one embodiment, T cells are induced that recognize the epitope NYNYLYRLF (SEQ ID NO: 38). In one embodiment, T cells are induced that recognize the epitope QYIKWPWYI (SEQ ID NO: 37). In one embodiment, T cells are induced that recognize the epitope KCYGVSPTK (SEQ ID NO: 45). In one embodiment, T cells are induced that recognize the epitope RLQSLQTYV (SEQ ID NO: 36). In some embodiments, the methods and agents, e.g., mRNA compositions, described herein are administered according to a regimen which achieves such induction of T cells.

In some embodiments, the methods and agents, e.g., mRNA compositions, described herein following administration to a subject induce a cell-mediated immune response (e.g., CD4+ and/or CD8+ T cell response) that is detectable 15 weeks or later, 16 weeks or later, 17 weeks or later, 18 weeks or later, 19 weeks or later, 20 weeks or later, 21 weeks or later, 22 weeks or later, 23 weeks or later, 24 weeks or later or 25 weeks or later after administration, e.g., using two doses of RNA described herein (wherein the second dose may be administered about 21 days following administration of the first dose). In some embodiments, the methods and agents, e.g., mRNA compositions, described herein are administered according to a regimen which achieves such induction of a cell-mediated immune response.

In one embodiment, vaccination against Coronavirus described herein, e.g., using RNA described herein which may be administered in the amounts and regimens described herein, e.g., at two doses of 30 μg per dose e.g. administered 21 days apart, may be repeated after a certain period of time, e.g., once it is observed that protection against Coronavirus infection diminishes, using the same or a different vaccine as used for the first vaccination. Such certain period of time may be at least 6 months, 1 year, two years etc. In one embodiment, the same RNA as used for the first vaccination is used for the second or further vaccination, however, at a lower dose or a lower frequency of administration. For example, the first vaccination may comprise vaccination using a dose of about 30 ug per dose, wherein in one embodiment, at least two of such doses are administered, (for example, a second dose may be administered about 21 days following administration of the first dose) and the second or further vaccination may comprise vaccination using a dose of less than about 30 μg per dose, wherein in one embodiment, only one of such doses is administered. In one embodiment, a different RNA as used for the first vaccination is used for the second or further vaccination, e.g., BNT162b2 is used for the first vaccination and BNT162B1 or BNT162b3 is used for the second or further vaccination.

In one embodiment, the vaccination regimen comprises a first vaccination using at least two doses of compositions described herein, e.g., two doses of compositions described herein (wherein the second dose may be administered about 21 days following administration of the first dose), and a second vaccination using a single dose or multiple doses, e.g., two doses, of compositions described herein. In various embodiments, the second vaccination is administered 3 to 24 months, 6 to 18 months, 6 to 12 months, or 5 to 7 months after administration of the first vaccination, e.g., after the initial two-dose regimen. The total amount of RNA used in each dose of the second vaccination may be equal or different to the amount of RNA used in each dose of the first vaccination. In one embodiment, the total amount of RNA used in each dose of the second vaccination is equal to the total amount of RNA used in each dose of the first vaccination. In one embodiment, the total amount of RNA used in each dose of the second vaccination and the total amount of RNA used in each dose of the first vaccination is about 60 μg per dose. In one embodiment, the total amount of RNA used in each dose of the second vaccination and the total amount of RNA used in each dose of the first vaccination is about 90 μg per dose. In one embodiment, the same RNA as used for the first vaccination is used for the second vaccination. In one embodiment, the first vaccination and the second vaccination both administer a dose of a composition comprising an at least bivalent SARS-CoV-2 vaccine and an at least tetravalent influenza vaccine. In some embodiments, each of the doses of the first vaccination and the second vaccination comprises 30 μg of the at least bivalent SARS-CoV-2 vaccine and 30 μg of the at least tetravalent influenza vaccine. In some embodiments, each of the doses of the first vaccination and the second vaccination comprises 30 μg of the at least bivalent SARS-CoV-2 vaccine and 60 μg of the at least tetravalent influenza vaccine.

In one embodiment, the composition used for the first vaccination and for the second vaccination comprises BNT162b2.

In some embodiments, when the composition used for the first vaccination and for the second vaccination comprises BNT162b2, the aim is to induce an immune response that targets SARS-CoV-2 variants including, but not limited to, the Omicron (B.1.1.529) variant. Accordingly, in some embodiments, when the composition used for the first vaccination and for the second vaccination comprises BNT162b2, the aim is to protect a subject from infection with SARS-CoV-2 variants including, but not limited to, the Omicron (B.1.1.529) variant.

In one embodiment, a different composition as used for the first vaccination is used for the second vaccination. In one embodiment, a composition used for the first vaccination comprises BNT162b2 and the composition used for the second vaccination comprises RNA encoding a SARS-CoV-2 S protein of a SARS-CoV-2 variant strain, e.g., a strain discussed herein. In one embodiment, the composition used for the first vaccination comprises BNT162b2 and the composition used for the second vaccination comprises RNA encoding a SARS-CoV-2 S protein of a SARS-CoV-2 variant strain that is prevalent or rapidly spreading at the time of the second vaccination. In one embodiment, the composition used for the first vaccination comprises BNT162b2 and the composition used for the second vaccination comprises RNA encoding a SARS-CoV-2 S protein, an immunogenic variant thereof, or an immunogenic fragment of the SARS-CoV-2 S protein or the immunogenic variant thereof comprising one or more of the mutations described herein for S protein variants such as SARS-CoV-2 S protein variants, in particular naturally occurring S protein variants. In one embodiment, the composition used for the first vaccination comprises BNT162b2 and the composition used for the second vaccination comprises RNA encoding a SARS-CoV-2 S protein, an immunogenic variant thereof, or an immunogenic fragment of the SARS-CoV-2 S protein or the immunogenic variant thereof comprising one or more mutations, such as all mutations, of a SARS-CoV-2 S protein of a SARS-CoV-2 variant selected from the group consisting of VOC-202012/01, 501.V2, Cluster 5, B.1.1.248, and Omicron (B.1.1.529).

In one embodiment, the composition used for the first vaccination comprises an RNA comprising a nucleotide sequence encoding a polypeptide comprising an amino acid sequence with proline residue substitutions at positions corresponding to 986 and 987 of SEQ ID NO:1 and the composition used for the second vaccination comprises RNA comprising a nucleotide sequence that encodes a polypeptide comprising an amino acid sequence with alanine substitution at position 80, glycine substitution at position 215, lysine substitution at position 484, tyrosine substitution at position 501, valine substitution at position 701, phenylalanine substitution at position 18, isoleucine substitution at position 246, asparagine substitution at position 417, glycine substitution at position 614, deletions at positions 242 to 244, and proline substitutions at positions 986 and 987 of SEQ ID NO:1.

In one embodiment, the composition used for the first vaccination comprises an RNA encoding a polypeptide comprising an amino acid sequence with proline residue substitutions at positions 986 and 987 of SEQ ID NO:1 and the composition used for the second vaccination comprises an RNA encoding a polypeptide comprising an amino acid sequence with the following mutations in SEQ ID NO:1:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, as compared to SEQ ID NO: 1.

In one embodiment, the composition used for the first vaccination comprises an RNA that encodes a polypeptide comprising an amino acid sequence with following mutations in SEQ ID NO: 1: residue substitutions at positions 986 and 987 of SEQ ID NO:1 and the composition used for the second vaccination comprises RNA encoding a polypeptide comprising an amino acid sequence with the following mutations in SEQ ID NO:1:
T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, K986P, and V987P, as compared to SEQ ID NO: 1.

In one embodiment, the composition used for the first vaccination comprises RNA encoding a polypeptide comprising an amino acid sequence with proline residue substitutions at positions 986 and 987 of SEQ ID NO: 1 and the composition used for the second vaccination comprises RNA encoding a polypeptide comprising an amino acid sequence with the following mutations in SEQ ID NO:1:
T19I, Δ24-26, A27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, K986P, and V987P, as compared to SEQ ID NO: 1.

In one embodiment, the composition used for the first vaccination encodes a polypeptide comprising an amino acid sequence with following mutations in SEQ ID NO: 1:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, and the composition used for the second vaccination comprises an RNA that encodes a polypeptide comprising an amino acid sequence with the following mutations in SEQ ID NO: 1:
T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, K986P, and V987P, as compared to SEQ ID NO: 1.

In one embodiment, the composition used for the first vaccination encodes a polypeptide comprising an amino acid sequence with following mutations in SEQ ID NO: 1:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, and the composition used for the second vaccination comprises an RNA that encodes a polypeptide comprising an amino acid sequence with the following mutations in SEQ ID NO: 1:
T19I, Δ24-26, A27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, K986P, and V987P, as compared to SEQ ID NO: 1.

In one embodiment, the composition used for the first vaccination encodes a polypeptide comprising an amino acid sequence with following mutations in SEQ ID NO: 1:
T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, K986P, and V987P, as compared to SEQ ID NO: 1, and the composition used for the second vaccination comprises an RNA that encodes a polypeptide comprising an amino acid sequence with the following mutations in SEQ ID NO: 1:
T19I, Δ24-26, A27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, K986P, and V987P, as compared to SEQ ID NO: 1.

In one embodiment, the composition used for the first vaccination comprises an RNA that encodes a polypeptide comprising the amino acid sequence of SEQ ID NO: 7 and the composition used for the second vaccination comprises an RNA encoding a polypeptide comprising the amino acid sequence of SEQ ID NO: 49.

In one embodiment, the composition used for the first vaccination comprises an RNA comprising the nucleotide sequence of SEQ ID NO: 20 and the composition used for the second vaccination comprises an RNA comprising the nucleotide sequence of SEQ ID NO: 51.

In one embodiment, the composition used for the first vaccination comprises an RNA that encodes a polypeptide comprising the amino acid sequence of SEQ ID NO: 7 and the composition used for the second vaccination comprises RNA encoding a polypeptide comprising the amino acid sequence of SEQ ID NO: 55, 58 or 61.

In one embodiment, the composition used for the first vaccination comprises an RNA that comprises the nucleotide sequence of SEQ ID NO: 20 and the composition used for the second vaccination comprises an RNA comprising the nucleotide sequence of SEQ ID NO: 57, 60, or 63.

In one embodiment, the composition used for the first vaccination comprises an RNA that encodes a polypeptide comprising the amino acid sequence of SEQ ID NO: 58 and the composition used for the second vaccination comprises RNA encoding a polypeptide comprising the amino acid sequence of SEQ ID NO: 49, 55 or 61.

In one embodiment, the composition used for the first vaccination comprises RNA that comprises the nucleotide sequence of SEQ ID NO: 60 and the composition used for the second vaccination RNA comprising the nucleotide sequence of SEQ ID NO: 51, 57, or 63.

In one embodiment, the composition used for the first vaccination comprises an RNA that encodes a polypeptide comprising the amino acid sequence of SEQ ID NO: 49 and the composition used for the second vaccination comprises RNA encoding a polypeptide comprising the amino acid sequence of SEQ ID NO: 55 or 61.

In one embodiment, the composition used for the first vaccination comprises RNA comprising the nucleotide sequence of SEQ ID NO: 51 and the composition used for the second vaccination comprises RNA comprising the nucleotide sequence of SEQ ID NO: 57 or 63.

In one embodiment, the composition used for the first vaccination comprises RNA that encodes a polypeptide comprising the amino acid sequence of SEQ ID NO: 55 and the composition used for the second vaccination comprises RNA encoding a polypeptide comprising the amino acid sequence of SEQ ID NO: 61.

In one embodiment, the composition used for the first vaccination comprises RNA comprising the nucleotide sequence of SEQ ID NO: 57 and the composition used for the second vaccination comprises RNA comprising the nucleotide sequence of SEQ ID NO: 63.

In one embodiment, the composition used for the first vaccination comprises RNA that encodes a polypeptide comprising an amino acid sequence with proline residue substitutions at positions 986 and 987 of SEQ ID NO:1 and the composition used for the second vaccination comprises RNA encoding a polypeptide comprising an amino acid sequence with the following mutations in SEQ ID NO:1:
A67V, Δ69-70, T95I, G142D, Δ143-145, Δ211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, as compared to SEQ ID NO: 1. In some embodiments, the polypeptide encoded by the RNA used in the second vaccination further comprises proline residue substitutions at positions corresponding to 986 and 987 of SEQ ID NO:1.

In one embodiment, the composition used for the first vaccination comprises RNA that encodes a polypeptide comprising the amino acid sequence of SEQ ID NO: 7 and the composition used for the second vaccination comprises RNA encoding a polypeptide comprising the amino acid sequence of SEQ ID NO: 52.

In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 30 μg of RNA comprising a sequence encoding a polypeptide comprising the amino acid sequence of SEQ ID NO: 7, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7.

In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 50 μg of RNA comprising a sequence encoding a polypeptide comprising the amino acid sequence of SEQ ID NO: 7, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7.

In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 60 μg of RNA comprising a sequence encoding a polypeptide comprising the amino acid sequence of SEQ ID NO: 7, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7. In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 30 μg of RNA comprising a sequence encoding a polypeptide comprising the amino acid sequence of SEQ ID NO: 49, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7.

In one embodiment, a subject is administered a primary regimen comprising at least two 30 ug doses of RNA comprising a sequence encoding a polypeptide comprising the amino acid sequence of SEQ ID NO: 7, and a booster regimen comprising at least two doses of 30 μg of RNA comprising a sequence encoding a polypeptide comprising the amino acid sequence of SEQ ID NO: 49, wherein in some embodiments the two doses of the booster regimen are administered at least 2 months apart from each other (e.g., at least 3 months, at least 4 months, at least 5 months, or at least 6 months apart from each other). In some embodiments, such a subject may have previously been administered a 30 ug dose of RNA comprising a sequence encoding a polypeptide comprising the amino acid sequence of SEQ ID NO: 7 as a booster dose.

In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 50 μg of RNA comprising a nucleotide sequence that encodes the amino acid sequence of SEQ ID NO: 49, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7.

In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 60 μg of RNA comprising a nucleotide sequence that encodes the amino acid sequence of SEQ ID NO: 49, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7.

In some embodiments, a subject is administered a primary regimen comprising two doses of 30 ug of RNA (administered, e.g., about 21 days after one another), wherein each 30 ug dose of RNA comprises 15 ug of RNA comprising a nucleotide sequence encoding the amino acid sequence of SEQ ID NO: 7 and 15 ug of RNA comprising a nucleotide sequence encoding the amino acid sequence of SEQ ID NO: 49. In some embodiments, such a primary regimen is administered to a vaccine naive subject.

In some embodiments, a subject is administered a primary regimen comprising two doses of 30 ug of RNA (administered, e.g., about 21 days after one another), wherein each 30 ug dose of RNA comprises a nucleotide sequence encoding the amino acid sequence of SEQ ID NO: 49. In some embodiments, such a primary regimen is administered to a vaccine naive subject.

In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 30 ug of RNA, wherein the 30 ug of RNA comprises 15 ug of RNA comprising a nucleotide sequence encoding the amino acid sequence of SEQ ID NO: 7 and 15 ug of RNA comprising a nucleotide sequence encoding the amino acid sequence of SEQ ID NO: 49, wherein the two RNAs are optionally administered in the same composition, and wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7. In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 50 ug of RNA, wherein the 50 ug of RNA comprises 25 ug of RNA comprising a nucleotide sequence encoding the amino acid sequence of SEQ ID NO: 7 and 25 ug of RNA comprising a nucleotide sequence encoding the amino acid sequence of SEQ ID NO: 49, wherein the two RNAs are optionally administered in the same composition (e.g., a formulation comprising both RNAs), and wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7.

In one embodiment, a subject is administered a primary regimen comprising two 30 ug doses of RNA comprising a sequence encoding a polypeptide comprising the amino acid sequence of SEQ ID NO: 7, and a booster regimen comprising at least one dose of 60 ug of RNA, wherein the 60 ug of RNA comprises 30 ug of RNA comprising a nucleotide sequence encoding the amino acid sequence of SEQ ID NO: 7 and 30 ug of an RNA comprising a nucleotide sequence encoding the amino acid sequence of SEQ ID NO: 49, wherein the two RNAs are optionally administered in the same composition, and wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a sequence encoding a polypeptide comprising the amino acid sequence of SEQ ID NO: 7.

In one embodiment, the RNA used for the first vaccination comprises the nucleotide sequence of SEQ ID NO: 20 and the RNA used for the second vaccination is RNA comprising the nucleotide sequence of SEQ ID NO: 54.

In one embodiment, a subject is administered a primary regimen comprising two 30 ug doses of RNA comprising a nucleotide sequence of SEQ ID NO: 20, and a booster regimen comprising at least one dose of 30 ug of RNA comprising a nucleotide sequence of SEQ ID NO: 20, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 50 ug of RNA comprising a nucleotide sequence of SEQ ID NO: 20, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20 . . . .

In one embodiment, a subject is administered a primary regimen comprising two 30 ug doses of RNA comprising a nucleotide sequence of SEQ ID NO: 20, and a booster regimen comprising at least one dose of 60 ug of RNA comprising a nucleotide sequence of SEQ ID NO: 20, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 30 ug of RNA comprising a nucleotide sequence of SEQ ID NO: 51, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 50 ug of RNA comprising a nucleotide sequence of SEQ ID NO: 51, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 60 ug of RNA comprising a nucleotide sequence of SEQ ID NO: 51, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 30 ug of RNA, wherein the 30 ug of RNA comprises 15 ug of RNA comprising a nucleotide sequence of SEQ ID NO: 20 and 15 ug of RNA comprising a nucleotide sequence of SEQ ID NO: 51, wherein the two RNAs are optionally administered in the same composition, and wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose comprising 50 ug of a RNA, wherein the 50 ug of RNA comprises 25 ug of RNA comprising a nucleotide sequence of SEQ ID NO: 20 and 25 ug of RNA comprising a nucleotide sequence of SEQ ID NO: 51, wherein the two RNAs are optionally administered in the same composition, and wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose comprising 60 ug of RNA, wherein the 60 ug of RNA comprises 30 ug of an RNA comprising a nucleotide sequence of SEQ ID NO: 20 and 30 ug of an RNA comprising a nucleotide sequence of SEQ ID NO: 51, wherein the two RNAs are optionally administered in the same composition, and wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 30 ug of RNA comprising a nucleotide sequence of SEQ ID NO: 57, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 50 ug of RNA comprising a nucleotide sequence of SEQ ID NO: 57, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 60 ug of RNA comprising a nucleotide sequence of SEQ ID NO: 57, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 30 ug of RNA comprising a nucleotide sequence of SEQ ID NO: 60, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 50 ug of RNA comprising a nucleotide sequence of SEQ ID NO: 60, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 60 ug of RNA comprising a nucleotide sequence of SEQ ID NO: 60, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 30 ug of RNA comprising a nucleotide sequence of SEQ ID NO: 63, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 20, and a booster regimen comprising at least one dose of 50 ug of RNA comprising a nucleotide sequence of SEQ ID NO: 63, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, a subject is administered a primary regimen comprising two 30 ug doses of RNA comprising a nucleotide sequence of SEQ ID NO: 63, and a booster regimen comprising at least one dose of 60 ug of RNA comprising a nucleotide sequence of SEQ ID NO: 57, wherein the booster regimen is administered at least 2 months (e.g., at least 3 months, at least 4 months, at least 5 months, or at least 6 months) after administration of the primary regimen, and wherein the subject has optionally previously been administered a first booster regimen comprising a 30 ug dose of RNA comprising a nucleotide sequence of SEQ ID NO: 20.

In one embodiment, the vaccination regimen comprises a first vaccination using two doses of RNA encoding a polypeptide comprising an amino acid sequence with proline residue substitutions at positions 986 and 987 of SEQ ID NO: 1 administered about 21 days apart and a second vaccination using a single dose or multiple doses of RNA encoding a polypeptide comprising an amino acid sequence with proline residue substitutions at positions 986 and 987 of SEQ ID NO:1 administered about 4 to 12 months, 5 to 12 months, or 6 to 12 months after administration of the first vaccination, i.e., after the initial two-dose regimen. In one embodiment, each RNA dose comprises 30 ug RNA. In this embodiment, the aim in one embodiment is to induce an immune response that targets SARS-CoV-2 variants including, but not limited to, the Omicron (B.1.1.529) variant. Accordingly, in this embodiment, the aim in one embodiment is to protect a subject from infection with SARS-CoV-2 variants including, but not limited to, the Omicron (B.1.1.529) variant.

In one embodiment, the vaccination regimen comprises a first vaccination using two doses of RNA encoding a polypeptide comprising an amino acid sequence with proline residue substitutions at positions 986 and 987 of SEQ ID NO: 1 administered about 21 days apart and a second vaccination using a single dose or multiple doses of RNA encoding a polypeptide comprising an amino acid sequence with alanine substitution at position 80, glycine substitution at position 215, lysine substitution at position 484, tyrosine substitution at position 501, valine substitution at position 701, phenylalanine substitution at position 18, isoleucine substitution at position 246, asparagine substitution at position 417, glycine substitution at position 614, deletions at positions 242 to 244, and proline substitutions at positions 986 and 987 of SEQ ID NO:1 administered about 6 to 12 months after administration of the first vaccination, i.e., after the initial two-dose regimen. In one embodiment, each RNA dose comprises 30 μg RNA.

In one embodiment, the vaccination regimen comprises a first vaccination using two doses of RNA encoding a polypeptide comprising an amino acid sequence with proline residue substitutions at positions 986 and 987 of SEQ ID NO: 1 administered about 21 days apart and a second vaccination using a single dose or multiple doses of RNA encoding a polypeptide comprising an amino acid sequence with the following mutations in SEQ ID NO:1:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, as compared to SEQ ID NO: 1, administered after, e.g., about 6 to 12 months after administration of the first vaccination, i.e., after the initial two-dose regimen.

In one embodiment, each RNA dose comprises 30 μg RNA.

In one embodiment, a vaccination regimen comprises a first vaccination using two doses of RNA encoding a polypeptide comprising an amino acid sequence with proline residue substitutions at positions 986 and 987 of SEQ ID NO: 1 administered about 21 days apart and a second vaccination using a single dose or multiple doses of RNA encoding a polypeptide comprising an amino acid sequence with the following mutations in SEQ ID NO:1:
A67V, Δ69-70, T95I, G142D, Δ143-145, Δ211, L212I, ins214EPE, G339D, S371L, S373P, S375F, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, as compared to SEQ ID NO: 1, administered after, e.g., about 6 to 12 months after administration of the first vaccination, i.e., after the initial two-dose regimen. In one embodiment, each RNA dose comprises 30 μg RNA. In some embodiments, the encoded polypeptide further comprises proline residue substitutions at positions corresponding to 986 and 987 of SEQ ID NO:1.

In one embodiment, the vaccination regimen comprises a first vaccination involving at least two doses of RNA encoding a polypeptide comprising an amino acid sequence with proline residue substitutions at positions 986 and 987 of SEQ ID NO: 1 administered about 21 days apart and a second vaccination involving a single dose or multiple doses of RNA encoding a polypeptide comprising an amino acid sequence with the following mutations in SEQ ID NO: 1:
T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, K986P, and V987P, as compared to SEQ ID NO: 1, administered after, e.g., about 6 to 12 months after administration of the first vaccination, i.e., after the initial two-dose regimen. In one embodiment, each or at least one RNA dose comprises 30 μg RNA.

In one embodiment, the vaccination regimen comprises a first vaccination involving at least two doses of RNA encoding a polypeptide comprising an amino acid sequence with proline residue substitutions at positions 986 and 987 of SEQ ID NO: 1 administered about 21 days apart and a second vaccination involving a single dose or multiple doses of RNA encoding a polypeptide comprising an amino acid sequence with the following mutations in SEQ ID NO: 1:
T19I, Δ24-26, A27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, K986P, and V987P, as compared to SEQ ID NO: 1 administered after, e.g., about 6 to 12 months after administration of the first vaccination, i.e., after the initial two-dose regimen. In one embodiment, each or at least one RNA dose comprises 30 μg RNA.

In one embodiment, the vaccination regimen comprises a first vaccination involving at least two doses of RNA encoding a polypeptide comprising an amino acid sequence with the following mutations in SEQ ID NO:1:
A67V, Δ69-70, T95I, G142D, Δ143-145, Δ211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, wherein the two doses of the first vaccination are administered about 21 days apart and wherein the vaccination regimen comprises a second vaccination involving a single dose or multiple doses of RNA encoding a polypeptide comprising an amino acid sequence with the following mutations in SEQ ID NO:1:
T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, K986P, and V987P, as compared to SEQ ID NO: 1 administered after, e.g., about 6 to 12 months after administration of the first vaccination, i.e., after the initial two-dose regimen. In one embodiment, each or at least one RNA dose comprises 30 μg RNA.

In one embodiment, the vaccination regimen comprises a first vaccination involving at least two doses of RNA encoding a polypeptide comprising an amino acid sequence with the following mutations in SEQ ID NO:1:
A67V, Δ69-70, T95I, G142D, Δ143-145, Δ211, L212I, ins214EPE, G339D, S371L, S373P, S375F, K417N, N440K,
G446S, S477N, T478K, E484A, Q493R, G496S, Q498R, N501Y, Y505H, T547K, D614G, H655Y, N679K, P681H, N764K, D796Y, N856K, Q954H, N969K, L981F, K986P and V987P, wherein the two doses of the first vaccination are administered about 21 days apart and wherein the vaccination regimen comprises a second vaccination involving a single dose or multiple doses of RNA encoding a polypeptide comprising an amino acid sequence with the following mutations in SEQ ID NO:1:
T19I, Δ24-26, A27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, K986P, and V987P, as compared to SEQ ID NO: 1 administered after, e.g., about 6 to 12 months after administration of the first vaccination, i.e., after the initial two-dose regimen. In one embodiment, each or at least one RNA dose comprises 30 μg RNA.

In one embodiment, the vaccination regimen comprises a first vaccination involving at least two doses of RNA encoding a polypeptide comprising an amino acid sequence with the following mutations in SEQ ID NO:1:
T19I, Δ24-26, A27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, K986P, and V987P, as compared to SEQ ID NO: 1, wherein the two doses of the first vaccination are administered about 21 days apart and wherein the vaccination regimen comprises a second vaccination involving a single dose or multiple doses of RNA encoding a polypeptide comprising an amino acid sequence with the following mutations in SEQ ID NO:1:
T19I, Δ24-26, A27S, Δ69/70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, K986P, and V987P, as compared to SEQ ID NO: 1 administered after, e.g., about 6 to 12 months after administration of the first vaccination, i.e., after the initial two-dose regimen. In one embodiment, each or at least one RNA dose comprises 30 μg RNA.

In one embodiment, a vaccination regimen comprises (i) a first vaccination comprising at least three doses of an RNA described herein (e.g., where each dose comprises about 30 ug of an RNA comprising a nucleotide sequence of SEQ ID NO: 20), wherein a second dose may be administered about 21 days following administration of a first dose, and a third dose may be administered at least about 1, 2, 3, 4, 5, 6, 7, 8, 9, 10, 11, or 12 months after a second dose; and (ii) a second vaccination comprising at least one dose of an RNA described herein (e.g., wherein each dose comprises about 30 μg RNA per dose). In some embodiments, a second vaccination comprises at least one dose of a bivalent vaccine described herein, e.g., about 30 μg total of a bivalent vaccine, e.g., a bivalent vaccine comprising about 15 μg RNA encoding a SARS-CoV-2 S protein from a Wuhan strain and about 15 μg RNA encoding a SARS-CoV-2 S protein comprising mutations characteristic of an Omicron variant (b2+Omi). In some embodiments, the bivalent vaccine comprises about 15 μg RNA encoding a SARS-CoV-2 S protein from a Wuhan strain and about 15 μg RNA encoding a SARS-CoV-2 S protein comprising mutations characteristic of a BA.1 Omicron variant (e.g., 15 μg RNA comprising a sequence of SEQ ID NO: 20 and 15 μg of RNA comprising a sequence of SEQ ID NO: 51). In some embodiments, the bivalent vaccine comprises about 15 μg RNA encoding a SARS-CoV-2 S protein from a Wuhan strain and about 15 μg RNA encoding a SARS-CoV-2 S protein comprising mutations characteristic of a BA.4/5 Omicron variant (e.g., 15 μg RNA comprising a sequence of SEQ ID NO: 20 and 15 μg of RNA comprising a sequence of SEQ ID NO: 72). In some embodiments, the vaccination regimen is administered to a subject who is at least about 12 years old. In some embodiments, the vaccination regimen is administered to a subject who is at least about 6 months old to less than about 12 years old.

In one embodiment, the second vaccination results in a boosting of the immune response.

In one embodiment, RNA described herein is co-administered with other vaccines. In some embodiments, RNA described herein is co-administered with a composition comprising one or more T-cell epitopes of SARS-CoV-2 or RNA encoding the same. In some embodiments, RNA described herein is co-administered with one or more T-cell epitopes, or RNA encoding the same, derived from an M protein, an N protein, and/or an ORF1ab protein of SARS-CoV-2, e.g., a composition disclosed in WO2021188969, the contents of which is incorporated by reference herein in its entirety. In some embodiments, compositions described herein (e.g., compositions comprising RNA encoding one or more SARS-CoV-2 S proteins and RNA encoding one or more influenza HA proteins) comprise and/or are co-administered with a T-string construct described in WO2021188969 (e.g., an RNA encoding SEQ ID NO: RS C7p2full of WO2021/188969). In some embodiments, compositions described herein and a T-string construct described in WO2021188969 are administered in a combination of up to about 100 ug RNA total. In some embodiments, subjects are administered with at least 2 doses of compositions described herein (e.g., in some embodiments at 60 g or 90 ug total RNA for each dose) in combination with a T-string construct (e.g., an RNA encoding SEQ ID NO: RS C7p2full of WO2021/188969), e.g., each dose of a combination of RNA described herein and an RNA encoding SEQ ID NO: RS C7p2full of up to about 100 ug RNA total, wherein the two doses are administered, for example, at least 4 weeks or longer (including, e.g., at least 5 weeks, at least 6 weeks, at least 7 weeks, at least 8 weeks, at least 9 weeks, at least 10 weeks, at least 11 weeks, or at least 12 weeks, or longer) apart from one another. In some embodiments, subjects are administered at least 3 doses of compositions described herein (e.g., in some embodiments at 60 ug or 90 ug total RNA for each dose) in combination with a T-string construct (e.g., an RNA encoding SEQ ID NO: RS C7p2full of WO2021/188969), e.g., each dose of a combination of RNA described herein and an RNA encoding SEQ ID NO: RS C7p2full of up to about 100 ug RNA total, wherein the first and the second doses and the second and third doses are each independently administered at least 4 weeks or longer (including, e.g., at least 5 weeks, at least 6 weeks, at least 7 weeks, at least 8 weeks, at least 9 weeks, at least 10 weeks, at least 11 weeks, or at least 12 weeks, or longer) apart from one another. In some embodiments, RNA described herein and the T-string construct may be co-administered as separate formulations (e.g., formulations administered on the same day to separate injection sites). In some embodiments, RNA described herein and the T-string construct may be co-administered as a co-formulation (e.g., a formulation comprising RNA described herein and the T-string construct as separate LNP formulations or as LNP formulations comprising both a T-string construct and RNA described herein).

In some embodiments, an RNA composition described herein is co-administered with one or more vaccines against a non-SARS-CoV-2 disease. In some embodiments, an RNA composition described herein is co-administered with one or more vaccines against a non-SARS-CoV-2 viral disease. In some embodiments, an RNA composition described herein is co-administered with one or more vaccines against a non-SARS-CoV-2 respiratory disease. In some embodiments, the non-SARS-CoV-2 respiratory disease is caused by a non-SARS-CoV-2 Coronavirus, an Influenza virus, a Pneumoviridae virus, or a Paramyxoviridae virus. In some embodiments, the Pneumoviridae virus is a Respiratory syncytial virus (RSV) or a Metapneumovirus. In some embodiments, the Metapneumovirus is a human metapneumovirus (hMPV). In some embodiments, the Paramyxoviridae virus is a Parainfluenza virus or a Henipavirus. In some embodiments the parainfluenzavirus is PIV3. In some embodiments, the non-SAR-COV-2 coronavirus is a betacoronavirus (e.g., SARS-CoV-1). In come embodiments the non-SARS-CoV-2 coronavirus is a Merbecovirus (e.g., a MERS-COV virus).

In some embodiments, an RNA composition described herein (e.g., (a) an RNA composition comprising one or more RNAs, each encoding a SARS-CoV-2 S protein, or (b) an RNA composition comprising (i) one or more RNAs, each encoding an antigen of a SARS-CoV-2 virus, and (ii) one or more RNAs, each encoding an antigen of an influenza virus) is co-administered with or further comprises an RSV vaccine (e.g., a vaccine that delivers an antigen of an RSV A and/or RSV B virus). In some embodiments, an RSV vaccine comprises an RSV fusion protein (F), an RSV attachment protein (G), an RSV small hydrophobic protein (SH), an RSV matrix protein (M), an RSV nucleoprotein (N), an RSV M2-1 protein, an RSV Large polymerase (L), and/or an RSV phosphoprotein (P), or an immunogenic fragment of immunogenic variant thereof, or a nucleic acid (e.g., RNA), encoding any one of the same or an immunogenic fragment or immunogenic variant of any one of the same.

Numerous RSV vaccines are known in the art, any one of which can be combined and/or co-administered with an RNA composition described herein. See, for example, the list of RSV vaccines provided on the website of PATH, a global health organization (see https_//www.path.org/resources/rsv-vaccine-and-mab-snapshot/), as well as in Mazur, Natalie I., et al, “The respiratory syncytial virus vaccine landscape: lessons from the graveyard and promising candidates,” The Lancet Infectious Diseases 18.10 (2018): e295-e311, the contents of each of which is incorporated by reference herein. In some embodiments, an RNA composition described herein is co-administered or combined with an RSV vaccine that has been previously published on (e.g., an RSV vaccine described on the PATH website page linked to above, or in Mazur et al.). In some embodiments, an RNA composition described herein is co-administered or combined with a live-attenuated or chimeric vaccine (e.g., rBCG-N-hRSV (developed by Ponteificia Uinersidad Catolica de Chile), RSV D46 cp ΔM202 (developed by Sanofi Pasteur/LID/NIAD/NIH), RSV LID ΔM2-2 1030s (developed by Sanofi Pasteur/LID/NIAD/NIH), RSV ΔNS2 Δ1313/I1314L (developed by Sanofi Pasteur/LID/NIAD/NIH), RSV D46 ΔNS2 N ΔM2-2 HindIII (developed by Sanofi Pasteur/LID/NIAD/NIH) or RSV LID ΔM2-2 1030s (developed by Sanofi Pasteur/LID/NIAD/NIH), MV-012-968 (developed by Meissa Vaccines), SP0125 (developed by Sanofi), blb201 (developed by Blue lake), CodaVax™-RSV (developed by Cadagenix), RSVDeltaG (developed by Intravacc), or SeVRSV (developed by SIHPL and St. Jude hospital), a particle based vaccine (e.g., RSV F nanoparticle (developed by Novavax) or SynGEM (developed by Mucosis), Icosavzx (developed by IVX-121), or V-306 (developed by Virometix)), a subunit vaccine (e.g., GSK RSV F (developed by GSK), Arexvy (developed by GSK), DPX-RSV (developed by Dalousie University, Immunovaccine, and VIB), RSV F DS-Cav1 (developed by NIH/NIAID/VRC), MEDI-7510 (developed by MedImmune), RSVpreF (developed by Pfizer, also known as Abrysvo™), ADV110 (developed by Advaccine), VN-0200 (developed by Daiichi Sankyo, Inc.)), a vector vaccine (e.g., MVA-BN RSV (developed by Banarian Nordic), VXA-RSVf oral (developed by Vaxart), Ad26.RSV.pref (developed by Janssen), ChAd155-RSV (developed by GSK) Immunovaccine, DPX-RSV (developed by VIB), or DS-Cav1 (developed by NIH/NIAID/VRC) or a nucleic acid vaccine (e.g., an mRNA vaccine being developed by CureVac (currently unnamed) or mRNA-1345 (developed by Moderna), or SP0274 (developed by Sanofi)).

In some embodiments, an RSV vaccine delivers an F protein (e.g., a prefusion stabilized F protein), or an immunogenic fragment thereof (e.g., an ectodomain) of a single RSV subtype (e.g., an RSV A subtype or an RSV B subtype). In some embodiments, an RSV vaccine delivers an F protein (e.g., a prefusion stabilized F protein), or an immunogenic fragment thereof, of two or more RSV subtypes (e.g., one or more RSV A subtypes and/or one or more RSV B subtypes)). In some embodiments, an RSV vaccine is a bivalent vaccine that delivers an F protein of an A subtype and an F protein of a B subtype, or immunogenic fragments thereof.

Mutations that stabilize a prefusion confirmation of an RSV F protein are known in the art (e.g., as described in WO2017/109629A1; WO2020/026147A1; McLellan, Jason S., et al. “Structure-based design of a fusion glycoprotein vaccine for respiratory syncytial virus,” science 342.6158 (2013): 592-598; and Che, Ye, et al. “Rational design of a highly immunogenic prefusion-stabilized F glycoprotein antigen for a respiratory syncytial virus vaccine,” Science Translational Medicine 15.693 (2023): eade6422; and Crank, Michelle C., et al. “A proof of concept for structure-based vaccine design targeting RSV in humans.” Science 365.6452 (2019): 505-509; the contents of each of which are incorporated by reference herein in their entirety). In some embodiments, an RSV F protein, or an immunogenic fragment thereof, comprises one or more of: one or more stabilizing engineered disulfide bonds (e.g., mutations corresponding to T103C-I148C), one or more cavity filling mutations (e.g., a mutation at a position corresponding to S190 (e.g., S190I)), and one or more charge neutralization mutations (e.g., a mutation at a position corresponding to D486 (e.g., D486S)). In some embodiments, an RSV vaccine comprise one or more stabilizing engineered disulfide bonds (e.g., mutations corresponding to S155C-S290C), and two cavity-filing mutations (e.g., mutations at positions corresponding to S190 and V296 (e.g., S190F and V296F). In some embodiments, an RSV F protein, or an immunogenic fragment thereof comprises one or more modifications present in RSVpreF (e.g., as described in Che, Ye, et al. “Rational design of a highly immunogenic prefusion-stabilized F glycoprotein antigen for a respiratory syncytial virus vaccine.” Science Translational Medicine 15.693 (2023): eade6422.) In some embodiments, an RSV F protein, or an immunogenic fragment thereof, comprises one or modifications present in DSCav-1 (e.g., as described in Crank, Michelle C., et al. “A proof of concept for structure-based vaccine design targeting RSV in humans.” Science 365.6452 (2019): 505-509).

In some embodiments, an RSV vaccine delivers one or more F proteins (e.g., one or more prefusion stabilized F proteins), or immunogenic fragments thereof, each of which comprises a heterologous domain that can induce trimerization (e.g., a T4 fibritin-derived trimization domain (also known as a “foldon” domain)).

In some embodiments, an RSV vaccine delivers one or more F proteins, or immunogenic fragments thereof, each of which comprise a mutation that disrupts a furin cleavage site and/or a pep27 cleavage site.

In some embodiments, an RSV vaccine comprises one or more antigens described in WO2017/109629A1, WO2020/026147A1, WO2010/149745A1, WO2010/149743A2, WO2010/006447A1, WO2009/079796A1, WO2008/114149A2, and WO2007/068907A2, the contents of each of which are incorporated by reference herein in their entirety.

In some embodiments an RSV vaccine does not include an adjuvant (e.g., an adjuvant separate from an RNA and/or an LNP that can also be present). In some embodiments, an RSV vaccine does include an adjuvant. In some embodiments, an RSV vaccine includes an adjuvant (e.g., 3-O-desacyl-4′-monophosphoryl lipid A (MPL) from Salmonella Minnesota, a saponin adjuvant (e.g., QS-21), or a combination thereof (e.g., where the combination is provided in a liposomal formulation)). In some embodiments, an RSV vaccine includes AS01E as an adjuvant. In some embodiments, an RSV vaccine includes an adjuvant as described in WO2022/248353A1, WO2018/104313A1, or WO2005/117958A1, the contents of each of which are hereby incorporated by reference here in their entirety.

In some embodiments, an RSV vaccine delivers a prefusion stabilized F protein or an immunogenic fragment thereof (e.g., an ectodomain stabilized in a prefusion confirmation). In some embodiments, an RSV vaccine is a bivalent vaccine that delivers stabilized prefusion F proteins, or immunogenic fragments thereof, from two RSV subtypes (e.g., comprising a sequence based on RSV A and RSV B subtypes, e.g., RSVpreF (also known as Abrysvo™), as described in Falsey A., et al. J. Infect Dis 2022; 225(12):2056-2066; Walsh E., et al. J. Infect Dis 2022; 225(8):1357-1366; Che, Ye, et al. “Rational design of a highly immunogenic prefusion-stabilized F glycoprotein antigen for a respiratory syncytial virus vaccine.” Science Translational Medicine 15.693 (2023): eade6422; and/or Baber J., et al. J. Infect Dis 2022 May 11; jiac189, the contents of each of which are hereby incorporated by reference in their entirety). In some embodiments, an RSV vaccine is a monovalent vaccine that delivers a prefusion stabilized F protein, or an immunogenic fragment thereof, of a single RSV subtype, and optionally further comprises an adjuvant (e.g., an RSV A or RSV B subtype (e.g., Arexvy™)).

In some embodiments, an RNA composition described herein (e.g., a composition comprising one or more RNAS, each encoding a SARS-CoV-2 S protein) is co-administered with an influenza vaccine (e.g., a non-RNA influenza vaccine). In some embodiments, the influenza vaccine is an alphainfluenza virus, a betainfluenza virus, a gammainfluenza virus or a deltainfluenza virus vaccine. In some embodiments the vaccine is an Influenza A virus, an Influenza B virus, an Influenza C virus, or an Influenza D virus vaccine. In some embodiments, the influenza A virus vaccine comprises a hemagglutinin selected from H1, H2, H3, H4, H5, H6, H7, H8, H9, H10, H11, H12, H13, H14, H15, H16, H17, and H18, or an immunogenic fragment or variant of the same, or a nucleic acid (e.g., RNA) encoding any one of the same. In some embodiments the influenza A vaccine comprises or encodes a neuraminidase (NA) selected from N1, N2, N3, N4, N5, N6, N7, N8, N9, N10, and N11, or an immunogenic fragment or variant of the same, or a nucleic acid (e.g., RNA) encoding any one of the same. In some embodiments, the influenza vaccine comprises at least one Influenza virus hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, and/or polymerase basic protein 2 (PB2), or an immunogenic fragment or variant thereof, or a nucleic acid (e.g., RNA) encoding any of one of the same.

In some embodiments, an RNA composition provided herein and other injectable vaccine(s) are administered at different times. In some embodiments, an RNA composition provided herein is administered at the same time as other injectable vaccine(s). In some such embodiments, an RNA composition provided herein and at least one another injectable vaccine(s) are administered at different injection sites. In some embodiments, an RNA composition provided herein is not mixed with any other vaccine in the same syringe. In some embodiments, an RNA composition provided herein is not combined with other coronavirus vaccines as part of vaccination against coronavirus, e.g., SARS-CoV-2. In some embodiments, an RNA composition provided herein and at least one other injectable vaccine(s) are administered at the same injection site.

In some embodiments, two or more vaccines (e.g., a SARS-CoV-2 vaccine and influenza vaccine described herein; a SARS-CoV-2 vaccine and an RSV vaccine described herein; or a SARS-CoV-2 vaccine, influenza vaccine, and an RSV vaccine described herein) are mixed immediately before administering to a subject. In some embodiments, two or more vaccines described herein are administered in a single shot and are not substantially mixed prior to injection (e.g., are administered using a method similar to that shown in FIG. 3). In some embodiments, two or more vaccines described herein are administered in a single shot using a dual chamber syringe (e.g., as described in Sousa, Liliana B., et al. “Brief report on double-chamber syringes patents and implications for infusion therapy safety and efficiency, International Journal of Environmental Research and Public Health 17.21 (2020): 8209, the contents of which are incorporated by referenced herein in their entirety). In some embodiments, the double chamber syringe comprises two vertical chambers (e.g., the Dual Chamber Reconstitution Syringe, provided by Credence MedSystems (e.g., as described at https_//www.credencemed.com/dual-chamber/).

In some embodiments, one or more vaccines to be administered to a subject are lyophilized. In such embodiments, the one or more lyophilized vaccines may be reconstituted in a liquid vaccine that is to be coadministered (e.g., in some embodiments, a lyophilized RSV vaccine is coadministered with a liquid SARS-CoV-2 vaccine and/or a liquid influenza vaccine, and the vaccines are coadministered by reconstituting the lyophilized RSV vaccine with the liquid SARS-CoV-2 and/or liquid influenza vaccine, and then administering to a subject).

Indeed, without wishing to be bound by any particular theory, the present disclosure proposes that, in some embodiments, certain advantages may be achieved by administering a plurality of immunogens (e.g., antigenic polypeptides and/or nucleic acids, e.g., RNAs, that encode them) to the same site and/or at the same time—e.g., in a single composition which may, in some embodiments, be a recently combined and/or mixed composition.

Those skilled in the art will appreciate various immunological rationales that might favor separate administration (e.g., in separate compositions and/or at separate sites and/or at different times) of different vaccines and/or vaccine components—for example to reduce immunological competition between or among immunogens. However, the present disclosure provides that, in many embodiments, different vaccines (and/or different components thereof), and in some embodiments all administered vaccines, may desirably be administered together, at the same site, at the same time (e.g., by passage through a common needle or port).

In some embodiments, an RNA composition provided herein (e.g., a SARS-CoV-2 RNA vaccine) and at least one other vaccine (e.g., a commercially available influenza vaccine) are administered at the same time, using the same syringe (e.g., administered by (i) taking a first composition (e.g., a SARS-CoV-2 RNA vaccine or a commercially available influenza vaccine) from a first container in a syringe, (ii) taking a second composition (e.g., a SARS-CoV-2 RNA vaccine or a commercially available influenza vaccine) from a second container in the same syringe (still comprising the first composition), and (iii) administering to a subject (e.g., administering without mixing and/or inverting).

In some embodiments, an RNA composition provided herein (e.g., a SARS-CoV-2 RNA vaccine) and an RSV vaccine are administered at the same time, using the same syringe (e.g., administered by (i) taking a first composition (e.g., a SARS-CoV-2 RNA vaccine or an RSV vaccine (e.g., RSVpref) from a first container in a syringe, (ii) taking a second composition (e.g., a SARS-CoV-2 RNA vaccine or an RSV vaccine (e.g., RSVpref) from a second container in the same syringe (still comprising the first composition), and (iii) administering to a subject (e.g., administering without mixing and/or inverting).

In some embodiments, an RNA composition comprising (i) one or more RNAs, each encoding a SARS-CoV-2 S protein, and (ii) one or more RNAs, reaching encoding an influenza HA antigen, is administered with at least one other vaccine (e.g., an RSV vaccine) at the same time, using the same syringe (e.g., administered by (a) taking the RNA composition comprising (i) one or more RNAs, each encoding a SARS-CoV-2 S protein and (ii) one or more RNAs, each encoding an influenza HA antigen from a first container in a syringe, and (b) taking an RSV vaccine (e.g., RSVpref) from a second container in the same syringe (still comprising the RNA composition), and (c) administering to a subject (e.g., administering without mixing and/or inverting). In some embodiments, an RNA composition comprising (i) one or more RNAs, each encoding a SARS-CoV-2 S protein, and (ii) one or more RNAS, reaching encoding an influenza HA antigen, is administered with at least one other vaccine (e.g., an RSV vaccine) at the same time, using the same syringe (e.g., administered by (a) taking an RSV vaccine (e.g., RSVpref) from a first container in a syringe, and (b) taking the RNA composition comprising (i) one or more RNAs, each encoding a SARS-CoV-2 S protein and (ii) one or more RNAs, each encoding an influenza HA antigen from a second container in the same syringe (still comprising the RNA composition), and (c) administering to a subject (e.g., administering without mixing and/or inverting).

In some embodiments, an RNA composition provided herein (e.g., a SARS-CoV-2 RNA vaccine) and at least two other vaccines (e.g., a commercially available influenza vaccine and an RSV vaccine (e.g., RSVpref)) are administered at the same time, using the same syringe (e.g., administered by (i) taking a first composition (e.g., a SARS-CoV-2 RNA vaccine, a commercially available influenza vaccine, or an RSV vaccine (e.g., RSVpref) from a first container in a syringe, (ii) taking a second composition (e.g., a SARS-CoV-2 RNA vaccine, a commercially available influenza vaccine, or an RSV vaccine (e.g., RSVpref) from a second container in the same syringe (still comprising the first composition), (iii) taking a third composition (e.g., a SARS-CoV-2 RNA vaccine, a commercially available influenza vaccine, or an RSV vaccine (e.g., RSVpref)) from a third container in the same syringe (still comprising the first and second compositions), and (iv) administering to a subject (e.g., administering without mixing and/or inverting).

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder but may or may not have the disease or disorder. In many embodiments, the individual is a human being. Unless otherwise stated, the terms “individual” and “subject” do not denote a particular age, and thus encompass adults, elderlies, children, and newborns. In some embodiments, the term “subject” includes humans of age of at least 50, at least 55, at least 60, at least 65, at least 70, or older. In some embodiments, the term “subject” includes humans of age of at least 65, such as 65 to 80, 65 to 75, or 65 to 70. In embodiments of the present disclosure, the “individual” or “subject” is a “patient”.

The term “patient” means an individual or subject for treatment, in particular a diseased individual or subject.

In one embodiment of the present disclosure, the aim is to provide an immune response against coronavirus, and to prevent or treat coronavirus infection.

A pharmaceutical composition comprising RNA encoding a peptide or protein comprising an epitope may be administered to a subject to elicit an immune response against an antigen comprising said epitope in the subject which may be therapeutic or partially or fully protective. A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

As used herein, “immune response” refers to an integrated bodily response to an antigen or a cell expressing an antigen and refers to a cellular immune response and/or a humoral immune response. The immune system is divided into a more primitive innate immune system, and acquired or adaptive immune system of vertebrates, each of which contains humoral and cellular components.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to immune effector cells, in particular to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as virus-infected cells, preventing the production of more diseased cells.

An immune effector cell includes any cell which is responsive to vaccine antigen. Such responsiveness includes activation, differentiation, proliferation, survival and/or indication of one or more immune effector functions. The cells include, in particular, cells with lytic potential, in particular lymphoid cells, and are preferably T cells, in particular cytotoxic lymphocytes, preferably selected from cytotoxic T cells, natural killer (NK) cells, and lymphokine-activated killer (LAK) cells. Upon activation, each of these cytotoxic lymphocytes triggers the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells.

The term “effector functions” in the context of the present disclosure includes any functions mediated by components of the immune system that result, for example, in the neutralization of a pathogenic agent such as a virus and/or in the killing of diseased cells such as virus-infected cells. In one embodiment, the effector functions in the context of the present disclosure are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

The term “immune effector cell” or “immunoreactive cell” in the context of the present disclosure relates to a cell which exerts effector functions during an immune reaction. An “immune effector cell” in one embodiment is capable of binding an antigen such as an antigen presented in the context of MHC on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present disclosure, “immune effector cells” are T cells, preferably CD4⁺ and/or CD8⁺ T cells, most preferably CD8⁺ T cells. According to the present disclosure, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

A “lymphoid cell” is a cell which is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted and preferably exerts effector functions of T cells.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4+ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8+ T cells since they express the CD8 glycoprotein on their surface. These cells recognize their targets by binding to antigen associated with MHC class I, which is present on the surface of nearly every cell of the body.

A majority of T cells have a T cell receptor (TCR) existing as a complex of several proteins. The TCR of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells. “Humoral immunity” or “humoral immune response” is the aspect of immunity that is mediated by macromolecules found in extracellular fluids such as secreted antibodies, complement proteins, and certain antimicrobial peptides. It contrasts with cell-mediated immunity. Its aspects involving antibodies are often called antibody-mediated immunity.

Humoral immunity refers to antibody production and the accessory processes that accompany it, including: Th2 activation and cytokine production, germinal center formation and isotype switching, affinity maturation and memory cell generation. It also refers to the effector functions of antibodies, which include pathogen neutralization, classical complement activation, and opsonin promotion of phagocytosis and pathogen elimination.

In humoral immune response, first the B cells mature in the bone marrow and gain B-cell receptors (BCR's) which are displayed in large number on the cell surface. These membrane-bound protein complexes have antibodies which are specific for antigen detection. Each B cell has a unique antibody that binds with an antigen. The mature B cells migrate from the bone marrow to the lymph nodes or other lymphatic organs, where they begin to encounter pathogens. When a B cell encounters an antigen, the antigen is bound to the receptor and taken inside the B cell by endocytosis. The antigen is processed and presented on the B cell's surface again by MHC-II proteins. The B cell waits for a helper T cell (TH) to bind to the complex. This binding will activate the TH cell, which then releases cytokines that induce B cells to divide rapidly, making thousands of identical clones of the B cell. These daughter cells either become plasma cells or memory cells. The memory B cells remain inactive here; later when these memory B cells encounter the same antigen due to reinfection, they divide and form plasma cells. On the other hand, the plasma cells produce a large number of antibodies which are released free into the circulatory system. These antibodies will encounter antigens and bind with them. This will either interfere with the chemical interaction between host and foreign cells, or they may form bridges between their antigenic sites hindering their proper functioning, or their presence will attract macrophages or killer cells to attack and phagocytose them.

The term “antibody” includes an immunoglobulin comprising at least two heavy (H) chains and two light (L) chains inter-connected by disulfide bonds. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. An antibody binds, preferably specifically binds with an antigen.

Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “antibody heavy chain”, as used herein, refers to the larger of the two types of polypeptide chains present in antibody molecules in their naturally occurring conformations.

An “antibody light chain”, as used herein, refers to the smaller of the two types of polypeptide chains present in antibody molecules in their naturally occurring conformations, K and A light chains refer to the two major antibody light chain isotypes.

The present disclosure contemplates an immune response that may be protective, preventive, prophylactic and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”.

The term “immunotherapy” relates to the treatment of a disease or condition by inducing, or enhancing an immune response. The term “immunotherapy” includes antigen immunization or antigen vaccination.

The terms “immunization” or “vaccination” describe the process of administering an antigen to an individual with the purpose of inducing an immune response, for example, for therapeutic or prophylactic reasons.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen” refers to any disease which implicates an antigen, e.g. a disease which is characterized by the presence of an antigen. The disease involving an antigen can be an infectious disease. As mentioned above, the antigen may be a disease-associated antigen, such as a viral antigen. In one embodiment, a disease involving an antigen is a disease involving cells expressing an antigen, preferably on the cell surface.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

### CERTAIN EXEMPLARY EMBODIMENTS

Embodiment 1. A vessel comprising a recently admixed combination comprising:
(a) a SARS-CoV-2 vaccine; and
(b) an influenza vaccine; wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the influenza vaccine: (i) is a nanoparticle (e.g., LNP) formulated RNA vaccine, or (ii) comprises one or more antigenic polypeptides (e.g., an HA protein) of one or more influenza virus strains.

Embodiment 2. A vessel comprising a recently admixed combination comprising:
(a) a SARS-CoV-2 vaccine; and
(b) an RSV vaccine;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains.

Embodiment 3. A vessel comprising a recently admixed combination comprising:
(a) a SARS-CoV-2 vaccine;
(b) an RSV vaccine;
(c) an influenza vaccine;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains; and
wherein the influenza vaccine: (i) is a nanoparticle (e.g., LNP) formulated RNA vaccine, or (ii) comprises one or more antigenic polypeptides (e.g., an HA protein) of one or more influenza virus strains.

Embodiment 4. The vessel of any one of embodiments 1-3, wherein the SARS-CoV-2 vaccine is a monovalent or bivalent vaccine.

Embodiment 5. The vessel of any one of embodiments 1, 3, or 4, wherein the influenza vaccine is a quadrivalent vaccine.

Embodiment 6. The vessel of any one of embodiments 1, 3, 4, or 5, wherein the influenza vaccine is an inactivated influenza virus, a recombinant influenza vaccine, a live attenuated influenza vaccine, a non-adjuvanted influenza vaccine, an adjuvanted influenza vaccine, or a subunit or split vaccine.

Embodiment 7. The vessel of any one of embodiments 2-6, wherein the RSV vaccine comprises a prefusion-stabilized F protein (or an immunogenic fragment thereof) of one or more RSV strains.

Embodiment 8. The vessel of any one of embodiments 1-7, wherein the vessel is a syringe or a vial.

Embodiment 9. A method of simultaneously vaccinating a human subject against each of SARS-CoV-2 and influenza, the method comprising:
simultaneously administering a SARS-CoV-2 vaccine composition and an influenza vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the influenza vaccine: (i) is a nanoparticle (e.g., LNP) formulated RNA vaccine, or (ii) comprises one or more antigenic polypeptides (e.g., an HA protein) of one or more influenza virus strains.

Embodiment 10. A method of simultaneously vaccinating a human subject against each of SARS-CoV-2 and RSV, the method comprising:
simultaneously administering a SARS-CoV-2 vaccine composition and an RSV vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains.

Embodiment 11. A method of simultaneously vaccinating a human subject against each of SARS-CoV-2, influenza, and RSV, the method comprising:
simultaneously administering a SARS-CoV-2 vaccine composition, an influenza vaccine composition, and an RSV vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs));
wherein the influenza vaccine: (i) is a nanoparticle (e.g., LNP) formulated RNA vaccine, or (ii) comprises one or more antigenic polypeptides (e.g., an HA protein) of one or more influenza virus strains; and
wherein the RSV vaccine comprises one or more antigenic polypeptides (e.g., an F protein or an immunogenic fragment thereof) associated with one or more RSV strains.

Embodiment 12. The method of embodiment 9, wherein the step of administering comprises injecting a composition through a needle or port; and
wherein the injected composition includes both the SARS-CoV-2 vaccine composition and the influenza vaccine composition; and
wherein the SARS-CoV-2 vaccine composition and the influenza vaccine composition are optionally administered using a syringe (e.g., a dual chamber syringe).

Embodiment 13. The method of embodiment 10, wherein the step of administering comprises injecting a composition through a needle or port; and
wherein the injected composition includes both the SARS-CoV-2 vaccine composition and the RSV vaccine composition; and
wherein the SARS-CoV-2 vaccine composition and the RSV vaccine composition are optionally administered using a syringe (e.g., a dual chamber syringe).

Embodiment 14. The method of embodiment 11, wherein the step of administering comprises injecting a composition through a needle or port;
wherein the injected composition includes each of the SARS-CoV-2 vaccine composition, the influenza vaccine composition, and the RSV vaccine composition; and
wherein the SARS-CoV-2 vaccine composition, the RSV vaccine composition, and the influenza vaccine composition are optionally administered using a syringe (e.g., a dual chamber syringe).

Embodiment 15. The method of embodiment 9 or 12, further comprising a step, prior to the step of administering, of admixing the SARS-CoV-2 vaccine composition and the influenza vaccine composition.

Embodiment 16. The method of embodiment 10 or 13, further comprising a step, prior to the step of administering, of admixing the SARS-CoV-2 vaccine composition and the RSV vaccine composition.

Embodiment 17. The method of embodiment 11 or 14, further comprising a step, prior to the step of administering, of admixing the SARS-CoV-2 vaccine composition, the influenza vaccine composition, and the RSV vaccine composition.

Embodiment 18. The method of any one of embodiments 15-17, wherein the step of admixing is performed within a period of time of the step of administering, which period of time is not more than 2 hours (e.g., not more than 1 hour, 30 minutes, 20 minutes, 15 minutes, 10 minutes, or 5 minutes).

Embodiment 19. The vessel of any one of embodiments 1-8 or the method of any one of embodiments 9-18, wherein the SARS-CoV-2 vaccine composition comprises two or more RNAs, each encoding an S protein of a different SARS-CoV-2 strain or variant, and wherein the two or more RNAs are encapsulated in separate populations of LNPs.

Embodiment 20. The vessel of any one of embodiments 1 and 3-8 or the method of any one of embodiments 9 and 11-18, or the vessel or method of embodiment 19, wherein the influenza vaccine comprises two or more RNAs (e.g., four RNAs), each encoding an antigenic polypeptide (e.g., HA protein) of a different influenza strain, and wherein the two or more RNAs are encapsulated in separate populations of LNPs.

Embodiment 21. The vessel of any one of embodiments 1-8, or the method of any one of embodiments 9-18, or the vessel or method of embodiment 19 or 20, wherein the SARS-CoV-2 vaccine comprises:
(a) (i) an RNA comprising a nucleotide sequence that includes modified uridines and encodes a first SARS-CoV-2 Spike (S) polypeptide from a SARS-CoV-2 strain, wherein the RNA encodes a polypeptide comprising an amino acid sequence that is at least 85% identical to SEQ ID NO: 7, and/or comprises a nucleotide sequence that is at least 85% identical to SEQ ID NO: 20 and/or a nucleotide sequence that is at least 85% identical to SEQ ID NO: 9, and (ii) an RNA comprising a nucleotide sequence that includes modified uridines and encodes a S polypeptide from an Omicron BA.4/5 SARS-CoV-2 variant, wherein the RNA comprises a nucleotide sequence that encodes a polypeptide comprising a sequence that is at least 85% identical to SEQ ID NO: 69 and/or wherein the RNA comprises a nucleotide sequence that is at least 85% identical to SEQ ID NO: 72 and/or a nucleotide sequence that is at least 85% identical to SEQ ID NO: 70; or
(b) an RNA comprising a nucleotide sequence that includes modified uridines and encodes a SARS-CoV-2 Spike (S) polypeptide, wherein the RNA comprises a nucleotide sequence that encodes a polypeptide comprising a sequence that is at least 85% identical to SEQ ID NO: 129 and/or wherein the RNA comprises a nucleotide sequence that is at least 85% identical to SEQ ID NO: 132 and/or a nucleotide sequence that is at least 85% identical to SEQ ID NO: 130.

Embodiment 22. The vessel of any one of embodiments 1-8, or the method of any one of embodiments 9-18, or the vessel or method of embodiment 19 or 20, wherein the influenza vaccine comprises:
(a) (i) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94 and/or a nucleotide sequence that is at least 85% identical to SEQ ID NO: 92; (ii) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99 and/or a nucleotide sequence that is at least 85% identical to SEQ ID NO: 97; (iii) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104 and/or a nucleotide sequence that is at least 85% identical to SEQ ID NO: 102; and (iv) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109 and/or a nucleotide sequence that is at least 85% identical to SEQ ID NO: 107; or
(b) (i) an RNA comprising a nucleotide sequence that includes modified uridines and encodes an influenza hemagglutinin antigen from an influenza A H1N1 strain, wherein the nucleotide sequence is at least 85% identical to SEQ ID NO: 92 and/or at least 85% identical to SEQ ID NO: 94; (ii) an RNA comprising a nucleotide sequence that includes modified uridines and encodes an influenza hemagglutinin antigen from an influenza A H3N2 strain, wherein the nucleotide sequence is at least 85% identical to SEQ ID NO: 82 and/or at least 85% identical to SEQ ID NO: 84; (iii) an RNA comprising a nucleotide sequence that includes modified uridines and encodes an influenza hemagglutinin antigen from an influenza B Victoria strain, wherein the nucleotide sequence is at least 85% identical to SEQ ID NO: 87 and/or that is at least 85% identical to SEQ ID NO: 89; and (iv) an RNA comprising a nucleotide sequence that includes modified uridines and encodes an influenza hemagglutinin antigen from an influenza B Yamagata strain, wherein the nucleotide sequence is at least 85% identical to SEQ ID NO: 107 and/or that is at least 85% identical to SEQ ID NO: 109.

Embodiment 23. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 9;
(ii) an RNA comprising a second nucleotide sequence that includes modified uridines and encodes a second SARS-CoV-2 Spike (S) polypeptide from a variant of the SARS-CoV-2 strain, wherein the second nucleotide sequence is at least 85% identical to SEQ ID NO: 70;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 92;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 97;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 102; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 107.

Embodiment 24. A composition comprising:
(i) an RNA comprising a nucleotide sequence that includes modified uridines and encodes a first SARS-CoV-2 Spike (S) polypeptide from a SARS-CoV-2 strain, wherein the RNA comprises a nucleotide sequence that is at least 85% identical to SEQ ID NO: 20;
(ii) an RNA comprising a nucleotide sequence that includes modified uridines and encodes a second SARS-CoV-2 Spike (S) polypeptide from a variant of the SARS-CoV-2 strain, wherein the RNA comprises a nucleotide sequence that is at least 85% identical to SEQ ID NO: 72;
(iii) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94;
(iv) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99;
(v) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104; and
(vi) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109.

Embodiment 25. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 9;
(ii) an RNA comprising a second nucleotide sequence that includes modified uridines and encodes a second SARS-CoV-2 Spike (S) polypeptide from a variant of the SARS-CoV-2 strain, wherein the second nucleotide sequence is at least 85% identical to SEQ ID NO: 70;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 92;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 82;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 87; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 107.

Embodiment 26. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 20;
(ii) an RNA comprising a second nucleotide sequence that includes modified uridines and encodes a second SARS-CoV-2 Spike (S) polypeptide from a SARS-CoV-2 strain, wherein the first nucleotide sequence is at least 85% identical to SEQ ID NO: 72;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 94;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 84;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 89; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 109.

Embodiment 27. The composition of any one of embodiments 23-26, wherein:
(a) the mass ratio of RNAS (i)-(ii) to RNAs (iii)-(vi) is 1:5 to 5:1, 1:4 to 4:1, 1:3 to 3:1, 1:2 to 2:1;
(b) the mass ratio of RNAS (iii)-(iv) to RNAs (v)-(vi) is 1:1 to 1:5; and/or
(c) the mass ratio of RNA (i) to RNA (ii) is 1:1.

Embodiment 28. The composition of any one of embodiments 23-27, wherein RNAs (iii), (iv), (v), and (vi) are present in a mass ratio of 1:1:1:1 or 1:1:5:5.

Embodiment 29. The composition of any one of embodiment 23-28, wherein the combined mass of RNAs (i)-(vi) is about 30 ug to about 100 ug.

Embodiment 30. The composition of any one of embodiments 23-29, wherein:
the combined mass of RNAs (i)-(ii) is about 3 μg to about 60 μg (e.g., about 3 μg, about 10 μg, about 30 μg, or about 60 μg); and/or
wherein the combined mass of RNAs (iii)-(vi) is about 30 μg to about 60 μg (e.g., about 30 μg or about 60 μg).

Embodiment 31. The composition of any one of embodiments 23-29, wherein:
(a) RNA (i) and (ii) are each present in an amount of about 15 μg, and RNAs (iii)-(vi) are each present in an amount of about 7.5 μg;
(b) RNA (i) and (ii) are each present in an amount of about 30 μg, and RNAs (iii)-(vi) are each present in an amount of about 7.5 μg;
(c) RNA (i) and (ii) are each present in an amount of about 15 μg, and RNAs (iii)-(vi) are each present in an amount of about 11.25 μg;
(d) RNA (i) and (ii) are each present in an amount of about 15 μg, RNAs (iii) and (iv) are each present in an amount of about 5 μg, and RNAs (v) and (vi) are each present in an amount of about 25 μg;
(e) RNA (i) and (ii) are each present in an amount of about 15 μg, RNAs (iii) and (iv) are each present in an amount of about 2.5 μg, and RNAs (v) and (vi) are each present in an amount of about 12.5 μg;
(f) RNA (i) and (ii) are each present in an amount of about 30 μg, RNAs (iii) and (iv) are each present in an amount of about 2.5 μg, and RNAs (v) and (vi) are each present in an amount of about 12.5 μg; or
(g) RNA (i)-(vi) are each present in an amount of about 15 μg.

Embodiment 32. A composition comprising:
(i) an RNA comprising a first nucleotide sequence that includes modified uridines and encodes a SARS-CoV-2 Spike (S) polypeptide, wherein the first nucleotide sequence is at least 85% identical to SEQ ID NO: 129;
(ii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 92;
(iii) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 99;
(iv) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 102; and
(v) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 107.

Embodiment 33. A composition comprising:
(i) an RNA comprising a nucleotide sequence that includes modified uridines and encodes a SARS-CoV-2 Spike (S) polypeptide, wherein the RNA comprises a nucleotide sequence that is at least 85% identical to SEQ ID NO: 132;
(ii) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94;
(iii) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99;
(iv) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104; and
(v) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109.

Embodiment 34. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 130;
(ii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 92;
(iii) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 82;
(iv) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 87; and
(v) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 107.

Embodiment 35. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 132;
(ii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 94;
(iii) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 84;
(iv) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 89; and
(v) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 109.

Embodiment 36. The composition of any one of embodiments 32-35, wherein:
RNA (i) and RNAs (ii)-(v) are present in a mass ratio of 1:5 to 5:1, 1:4 to 4:1, 1:3 to 3:1, or 1:2 to 2:1;
RNAs (ii) and (iii) and RNAs (iv) and (v) are present in a mass ratio of 1:1 to 1:5;
wherein RNAs (ii), (iii), (iv), and (v) are present in a mass ratio of 1:1:1:1 or 1:1:5:5.

Embodiment 37. The composition of any one of embodiment 24-36, wherein the combined mass of RNAs (i)-(v) is 30 ug to 100 ug.

Embodiment 38. The composition of any one of embodiments 32-37, wherein the mass of RNA (i) is about 3 μg to about 60 μg (e.g., about 3 μg, about 10 μg, about 30 μg, or about 60 μg), and/or wherein the combined mass of RNAs (ii)-(v) is about 30 μg to about 60 μg (e.g., about 30 μg or about 60 μg).

Embodiment 39. The composition of any one of embodiments 32-38, wherein:
(a) RNA (i) is present in an amount of about 30 μg, and RNAs (ii)-(v) are each present in an amount of about 7.5 μg;
(b) RNA (i) is present in an amount of about 60 μg, and RNAs (ii)-(v) are each present in an amount of about 7.5 μg;
(c) RNA (i) is present in an amount of about 30 μg, and RNAs (ii)-(v) are each present in an amount of about 11.25 μg;
(d) RNA (i) is present in an amount of about 30 μg, RNAs (ii) and (iii) are each present in an amount of about 5 μg, and RNAs (iv) and (v) are each present in an amount of about 25 μg;
(e) RNA (i) is present in an amount of about 30 μg, RNAs (ii) and (iii) are each present in an amount of about 2.5 μg, and RNAs (iv) and (v) are each present in an amount of about 12.5 μg;
(f) RNA (i) is present in an amount of about 30 μg, RNAs (ii) and (iii) are each present in an amount of about 2.5 μg, and RNAs (iv) and (v) are each present in an amount of about 12.5 μg; or
(g) RNA (i) is present in an amount of about 30 μg, and RNAs (ii)-(v) are each present in an amount of about 15 μg.

Embodiment 40. The composition of any one of embodiments 23-39, wherein the influenza A H1N1 strain is Influenza A/Wisconsin/588/2019 and wherein the influenza B Yamagata strain is Influenza B/PHUKET/3073/2013.

Embodiment 41. The composition of any one of embodiments 23, 24, 27-31, 33, 34, and 37-40, wherein the influenza A H3N2 strain is Influenza A/Cambodia/e0826360/2020, and wherein the influenza B Victoria strain is Influenza B/Washington/02/2019.

Embodiment 42. The composition of any one of embodiments 25-31 and 34-39, wherein the influenza A H3N2 strain is Influenza A/Darwin/6/2021

Embodiment 43. The composition of any one of embodiments 25-31 and 34-40, wherein the influenza A H3N2 strain is Influenza A/Darwin/6/2021 and/or wherein the influenza B Victoria strain is Influenza B/Austria/1359417/2021.

Embodiment 44. The composition of any one of embodiments 23-31, wherein the first SARS-CoV-2 Spike (S) polypeptide is from a Wuhan strain and wherein the second SARS-CoV-2 S polypeptide is from an Omicron BA.4/5 variant.

Embodiment 45. The composition of any one of embodiments 32-43, wherein the SARS-CoV-2 Spike (S) polypeptide is from an XBB.1.5 variant.

Embodiment 46. The composition of any one of embodiments 23-45, wherein each of the RNAs in the composition comprises the same non-coding elements that include the same 5′ cap, cap proximal sequence, 5′ UTR sequence, 3′ UTR sequence, and polyA sequence.

Embodiment 47. A composition comprising:
(i) a coronavirus RNA vaccine comprising one or more RNAs, each comprising a nucleotide sequence that encodes a SARS-CoV-2 antigen; and
(ii) an influenza RNA vaccine comprising one or more RNAs, each comprising one or more nucleotide sequences that encode an influenza antigen, wherein the influenza RNA vaccine encodes at least four influenza antigens, and wherein each influenza antigen is from a distinct influenza virus strain that is predicted to circulate during a flu season of a particular hemisphere;
wherein each RNA in the composition comprises the same non-coding elements, including the same 5′ cap, cap proximal sequence, 5′ UTR sequence, 3′ UTR sequence, and polyA sequence.

Embodiment 48. The composition of embodiment 47, wherein each of the one or more RNAs in the coronavirus RNA vaccine and each of the one or more RNAs in the influenza RNA vaccine include one or more modified uridines.

Embodiment 49. The composition of embodiment 47 or 48, wherein the at least four influenza antigens each are or comprise a hemagglutinin antigen from a distinct influenza virus strain predicted to circulate during a flu season of a particular hemisphere.

Embodiment 50. The composition of embodiment 49, wherein the distinct influenza virus is predicted to circulate during a flu season based on human serology data from the Northern or Southern hemisphere.

Embodiment 51. The composition of any one of embodiments 47-50, wherein the at least four influenza antigens are each encoded by a separate RNA.

Embodiment 52. The composition of any one of embodiments 47-51, wherein the coronavirus RNA vaccine encodes at least two SARS-CoV-2 antigens, each from a distinct SARS-CoV-2 strain or variant.

Embodiment 53. The composition of embodiment 52, wherein the at least two SARS-CoV-2 antigens are or comprise a SARS-CoV-2 Spike (S) polypeptide from a SARS-CoV-2 strain and a SARS-CoV-2 S polypeptide from a variant of the SARS-CoV-2 strain.

Embodiment 54. The composition of embodiment 52 or 53, wherein the at least two SARS-CoV-2 antigens are each encoded by a separate RNA.

Embodiment 55. The composition of any one of embodiments 47-54, wherein the RNAs in the coronavirus vaccine and the RNAs in the influenza vaccine are present in a mass ratio of 1:5 to 5:1, 1:4 to 4:1, 1:3 to 3:1, or 1:2 to 2:1.

Embodiment 56. The composition of any one of embodiments 47-55, wherein the at least four influenza antigens comprise at least two hemagglutinin antigens from influenza A viruses and at least two hemagglutinin antigens from influenza B viruses.

Embodiment 57. The composition of embodiment 56, wherein the RNAs that encode hemagglutinin antigens from influenza A viruses and the RNAs that encode hemagglutinin antigens from influenza B viruses are present in a mass ratio of 1:1 to 1:5 (e.g., 1:1 or 1:5).

Embodiment 58. The composition of any one of embodiments 54-57, wherein the at least two RNAs in the coronavirus vaccine are in a mass ratio of 1:1.

Embodiment 59. The composition of any one of embodiments 51-58, wherein the at least four RNAs in the influenza vaccine are present in a mass ratio of 1:1:1:1.

Embodiment 60. The composition of any one of embodiments 47-59, wherein the total amount of RNA in the composition is about 30 ug to about 100 ug (e.g., about 30 ug, about 45 ug, about 60 ug, about 75 ug, or about 90 ug).

Embodiment 61. A composition comprising:
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent, wherein the second infectious agent is different from the first infectious agent;
wherein each of the first and second RNAs in the composition comprises the same non-coding elements, including the same 5′ cap, cap proximal sequence, 5′ UTR sequence, 3′ UTR sequence, and polyA sequence, and
wherein at least one of the same non-coding elements is or comprises:
   (i) a 5′-UTR sequence that is or comprises a modified human alpha-globin 5′-UTR;
   (ii) a 3′-UTR sequence that is or comprises a first sequence from the amino terminal enhancer of split (AES) messenger RNA and a second sequence from the mitochondrial encoded 12S ribosomal RNA;
   (iii) a polyA sequence comprising at least 100 A nucleotides, wherein the first RNA and the second RNA each do not comprise a stretch of at least 30 contiguous C nucleotides between the 3′ UTR and the polyA sequence;
   (iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
   (v) a 5′ cap comprising a Cap1 structure and a cap proximal sequence comprising positions +1, +2, +3, +4, and +5 of the RNA; wherein:
      (a) the Cap1 structure comprises m7(3′OMeG)(5′)ppp(5′)(2′OMeA₁)pG₂, wherein A₁ is position +1 of the RNA, and G₂ is position +2 of the RNA; and
      (b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

Embodiment 62. A composition comprising:
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent that is different from the first infectious agent;
wherein each of the first and second RNAs in the composition comprises the same non-coding elements including the same 5′ cap, cap proximal sequence, 5′ UTR sequence, 3′ UTR sequence, and polyA sequence, and
wherein each of the first and second RNAs is characterized in that:
   (i) an immune response induced by the RNA in the composition has a level that is at least 80% of a level of an immune response induced by the same RNA when it is administered alone; and/or
   (ii) an immune response induced by the RNA in the composition has a level that is at least 80% of a level of an immune response induced by the same RNA when it is administered separately from the other RNAs at a different location of a subject's body; and/or
   (iii) an immune response induced by the RNA in the composition has a level that is at least 80% of a level of an immune response induced by a respective reference composition.

Embodiment 63. The composition of embodiment 62, wherein the respective reference composition is an inactivated virus vaccine.

Embodiment 64. The composition of embodiment 62 or 63, wherein the immune response induced by the one or more first RNA(s) and the one or more second RNA(s) are each at least 100% of a level of an immune response induced by the same RNA when the one or more first RNA(s) and the one or more second RNA(s) are administered separately.

Embodiment 65. The composition of any one of embodiments 62-64, wherein the immune response induced by the one or more first RNA(s) and the one or more second RNA(s) are each greater than an immune response induced by the same RNAs administered separately.

Embodiment 66. The composition of any one of embodiments 62-64, wherein the one or more first RNA(s) and the one or more second RNA(s) are each present at a dose that is lower than that of the same RNAs administered separately, wherein the immune response induced by the lower dose of the one or more first RNA(s) and the one or more second RNA(s) are each substantially comparable to or greater than the immune response induced by a greater dose of the same RNAs administered separately.

Embodiment 67. A composition comprising:
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent that is different from the first infectious agent;
wherein each of the first and second RNAs in the composition comprises the same non-coding elements, including the same 5′ cap, cap proximal sequence, 5′ UTR sequence, 3′ UTR sequence, and polyA sequence,
wherein each of the first and second RNAs is encapsulated, separately or together, in nanoparticles (e.g., each of the first RNAs is encapsulated in a first population of nanoparticles and each of the second RNAs is encapsulated in a second population of nanoparticles; or each of the first RNAs and each of the second RNAs is encapsulated in the same population of nanoparticles); and
wherein the composition is characterized in that:
   (i) RNA content of the composition is at least 95% that of the initial RNA content after storing for 24 hours;
   (ii) RNA encapsulation remains at least 95% that of the initial RNA encapsulation after storing for 24 hours;
   (iii) the nanoparticles encapsulating the first and second RNAs have maintained substantially the same size after storing for 24 hours;
   (iv) the nanoparticles encapsulating the first and second RNAs have maintained a polydispersity of no more than 0.3 after 24 hours; and/or
   (v) the mass ratio of the first RNA and the second RNA remains substantially the same after storing for 24 hours.

Embodiment 68. The composition of embodiment 67, wherein the nanoparticles comprise lipid nanoparticles, polyplexes (PLX), lipidated polyplexes (LPLX), liposomes, or polysaccharide nanoparticles.

Embodiment 69. The composition of embodiment 68, wherein the nanoparticles comprise lipid nanoparticles.

Embodiment 70. The composition of embodiment 69, wherein the lipid nanoparticles comprise: a cationically ionizable lipid, one or more neutral lipids, and a polymer-conjugated lipid.

Embodiment 71. The composition of embodiment 70, wherein the polymer-conjugated lipid comprises a PEG-conjugated lipid.

Embodiment 72. The composition of any one of embodiments 67-71, wherein the nanoparticles have an average diameter of about 50-150 nm.

Embodiment 73. The composition of any one of embodiments 67-72, wherein, for each of (i)-(v), the first 12 hours of storing is at 30° C. and the remaining 12 hours of storing is at 2-8° C.

Embodiment 74. The composition of any one of embodiments 67-73, wherein the one or more first RNAs comprise at least two first RNAs, each comprising a nucleotide sequence that encodes one or more antigenic polypeptides associated with a different strain or variant of the first infectious agent.

Embodiment 75. The composition of any one of embodiments 67-74, wherein the one or more second RNAs comprise at least two second RNAs, each comprising a nucleotide sequence that encodes one or more antigenic polypeptides associated with a different strain or variant of the second infectious agent.

Embodiment 76. The composition of any one of embodiments 67-75, wherein the one or more second RNAs comprise at least three second RNAs, each comprising a nucleotide sequence that encodes one or more antigenic polypeptides associated with a different strain or variant of the second infectious agent.

Embodiment 77. The composition of any one of embodiments 67-76, wherein the one or more second RNAs comprise at least four second RNAs, each comprising a nucleotide sequence that encodes one or more antigenic polypeptides associated with a different variant or strain of the second infectious agent.

Embodiment 78. A composition comprising:
a plurality of first RNAs each comprising a nucleotide sequence that encodes one or more antigenic polypeptides associated with a first infectious agent of a different strain and/or variant thereof;
one or more second RNAs each comprising a nucleotide sequence that encodes one or more antigenic polypeptides associated with a second infectious agent that is different from the first infectious agent;
and wherein each of the first and second RNAs is formulated, either separately or together, in the same nanoparticle formulation;
wherein (i) the first RNAs and the second RNAs are present in a mass ratio of 1:2 to 2:1 and/or (ii) the first RNAs and second RNAs are present in the total amount of about 10 μg to about 100 μg per dose; and
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent, wherein the second infectious agent is different from the first infectious agent;
wherein each of the first and second RNAs in the composition comprises the same non-coding elements, including the same 5′ cap, cap proximal sequence, 5′ UTR sequence, 3′ UTR sequence, and polyA sequence, and
wherein at least one of the same non-coding elements is or comprises:
   (i) a 5′-UTR sequence that is or comprises a modified human alpha-globin 5′-UTR;
   (ii) a 3′-UTR sequence that is or comprises a first sequence from the amino terminal enhancer of split (AES) messenger RNA and a second sequence from the mitochondrial encoded 12S ribosomal RNA;
   (iii) a polyA sequence comprising at least 100 A nucleotides, wherein the first RNA and the second RNA each do not comprise a stretch of at least 30 contiguous C nucleotides between the 3′ UTR and the polyA sequence;
   (iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
   (v) a 5′ cap comprising a Cap1 structure and a cap proximal sequence comprising positions +1, +2, +3, +4, and +5 of the RNA; wherein:
      (a) the Cap1 structure comprises m7(3′OMeG)(5′)ppp(5′)(2′OMeA₁)pG₂, wherein A₁ is position +1 of the RNA, and G₂ is position +2 of the RNA; and
(b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

Embodiment 79. The composition of any one of embodiments 61-78, wherein each of the first RNAs is co-formulated in the same nanoparticle formulation or wherein each of the first RNAs is formulated in separate nanoparticle formulations.

Embodiment 80. The composition of any one of embodiments 61-79, wherein each of the second RNAs is co-formulated in the same nanoparticle formulation or wherein each of the second RNAs is formulated in separate nanoparticle formulations.

Embodiment 81. The composition of any one of embodiments 61-80, wherein the first RNAs and the second RNAs are formulated in separate populations of nanoparticles.

Embodiment 82. The composition of any one of embodiments 61-81, wherein the first RNAs and the second RNAs are all co-formulated in the same nanoparticle formulation.

Embodiment 83. The composition of any one of embodiments 61-82, wherein the first infectious agent is or comprises a coronavirus.

Embodiment 84. The composition of embodiment 83, wherein the one or more first RNAs comprises (i) an RNA comprising a nucleotide sequence that encodes one or more antigenic polypeptides associated with a first coronavirus and (ii) an RNA comprising a nucleotide sequence that encodes one or more antigenic polypeptides associated with a second coronavirus.

Embodiment 85. The composition of any one of embodiments 61-84, wherein the one or more second RNAs comprise a plurality of second RNAs, each comprising a nucleotide sequence that encodes one or more antigenic polypeptides associated with a second infectious agent of a different strain and/or variant thereof.

Embodiment 86. The composition of any one of embodiments 61-85, wherein the one or more second RNAs comprise at least two second RNAs, each comprising a nucleotide sequence that encodes one or more antigenic polypeptides associated with a second infectious agent of a different strain and/or variant thereof.

Embodiment 87. The composition of any one of embodiments 61-86, wherein the one or more second RNAs comprise at least three second RNAs, each comprising a nucleotide sequence that encodes one or more antigenic polypeptides associated with a second infectious agent of a different strain and/or variant thereof.

Embodiment 88. The composition of any one of embodiments 61-87, wherein the one or more second RNAs comprise at least four second RNAs, each comprising a nucleotide sequence that encodes one or more antigenic polypeptides associated with a second infectious agent of a different strain and/or variant thereof.

Embodiment 89. The composition of any one of embodiments 61-88, wherein the second infectious agent is or comprises a bacterial infectious agent.

Embodiment 90. The composition of embodiment 89, wherein the bacterial infectious agent is Streptococcus pneumoniae.

Embodiment 91. The composition of any one of embodiments 61-88, wherein the second infectious agent is or comprises a viral infectious agent.

Embodiment 92. The composition of embodiment 91, wherein the second infectious agent is a viral infectious agent that induces an infectious respiratory disease.

Embodiment 93. The composition of embodiment 92, wherein the viral infectious agent is or comprises an influenza virus, a pneumoviridae virus, or a Paramyxoviridae virus.

Embodiment 94. The composition of embodiment 93, wherein the Pneumoviridae virus is a Respiratory syncytial virus (RSV).

Embodiment 95. The composition of embodiment 93, wherein the infectious respiratory disease is or comprises an influenza type A, type B, and/or type C virus.

Embodiment 96. The composition of embodiment 95, wherein the infectious respiratory disease is or comprises an influenza type A, and/or type B virus.

Embodiment 97. The composition of embodiment 96, wherein the one or more second RNAs comprise (i) at least one RNA comprising a nucleotide sequence that encodes one or more antigenic polypeptides associated with influenza type A virus and (ii) at least one RNA comprising a nucleotide sequence that encodes one or more antigenic polypeptides associated with influenza type B virus.

Embodiment 98. The composition of embodiment 96 or 97, wherein the one or more second RNAs comprise (i) at least two RNAs, each comprising a nucleotide sequence that encodes one or more antigenic polypeptides associated with a different strain of an influenza type A virus, and (ii) at least two RNAs, each comprising a nucleotide sequence that encodes one or more antigenic polypeptides associated with a different strain of an influenza type B virus.

Embodiment 99. The composition of any one of embodiments 95-98, wherein the antigenic polypeptide(s) associated with each influenza virus is/are each independently a Hemagglutinin (HA) polypeptide, a neuraminidase (NA) polypeptide, or combinations thereof, or immunogenic fragments thereof.

Embodiment 100. The composition of any one of embodiments 96-99, wherein the strain(s) of the influenza type A and influenza type B viruses have each been predicted to be or is a circulating strain in the coming flu season, for example, based on human serology data.

Embodiment 101. The composition of any one of embodiments 96-100, wherein the strain(s) of the influenza A virus are selected from an H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, and an H10N8 virus.

Embodiment 102. The composition of embodiment 101, wherein the strain(s) of the influenza type A virus is selected from an H1N1, H3N2, H5N1, and an H5N8 virus.

Embodiment 103. The composition of any one of embodiments 98-102, wherein the one or more second RNAs comprise an RNA comprising a nucleotide sequence encoding an antigenic polypeptide associated with an H1N1 virus.

Embodiment 104. The composition of embodiment 103, where the H1N1 virus is A/Wisconsin/588/2019.

Embodiment 105. The composition of embodiment 104, wherein the antigenic polypeptide associated with A/Wisconsin/588/2019 is an HA polypeptide and comprises an amino acid sequence that is at least 85% identical to SEQ ID NO: 90.

Embodiment 106. The composition of embodiment 104 or 105, wherein the antigenic polypeptide associated with A/Wisconsin/588/2019 is an HA polypeptide and the RNA encoding the HA polypeptide comprises a nucleotide sequence that is at least 85% identical to SEQ ID NO: 92.

Embodiment 107. The composition of any one of embodiments 98-106, wherein the one or more second RNAs comprise an RNA comprising a nucleotide sequence encoding an antigenic polypeptide associated with an H3N2 virus.

Embodiment 108. The composition of embodiment 107, wherein the H3N2 virus is A/Cambodia/e0826360/2020.

Embodiment 109. The composition of embodiment 108, wherein the antigenic polypeptide associated with A/Cambodia/e0826360/2020 is an HA polypeptide and comprises an amino acid sequence that is at least 85% identical to SEQ ID NO: 95.

Embodiment 110. The composition of embodiment 108 or 109, wherein the antigenic polypeptide associated with A/Cambodia/e0826360/2020 is an HA polypeptide, and the RNA encoding the HA polypeptide comprises a nucleotide sequence that is at least 85% identical to SEQ ID NO: 92.

Embodiment 111. The composition of embodiment 107, wherein the H3N2 virus is A/Darwin/6/2021.

Embodiment 112. The composition of embodiment 111, wherein the antigenic polypeptide associated with A/Darwin/6/2021 is an HA polypeptide and comprises an amino acid sequence that is at least 85% identical to SEQ ID NO: 80.

Embodiment 113. The composition of embodiment 111 or 112, wherein the antigenic polypeptide associated with A/Darwin/6/2021 is an HA polypeptide and the RNA encoding the HA polypeptide comprises a nucleotide sequence that is at least 85% identical to SEQ ID NO: 82.

Embodiment 114. The composition of any one of embodiments 98-113, wherein the one or more second RNAs comprise an RNA comprising a nucleotide sequence encoding an antigenic polypeptide associated with a B/Yamagata or B/Victoria lineage virus.

Embodiment 115. The composition of embodiment 114, where the B/Victoria lineage influenza virus is B/Washington/02/2019.

Embodiment 116. The composition of embodiment 115, wherein the antigenic polypeptide associated with B/Washington/02/2019 is an HA polypeptide and comprises a sequence that is at least 85% identical to SEQ ID NO: 100.

Embodiment 117. The composition of embodiment 115 or 116, wherein the antigenic polypeptide associated with B/Washington/02/2019 is an HA polypeptide, and the RNA encoding the HA polypeptide comprises a nucleotide sequence that is at least 85% identical to SEQ ID NO: 102.

Embodiment 118. The composition of embodiment 114, where the B/Victoria lineage influenza virus is B/Austria/1359417/2021.

Embodiment 119. The composition of embodiment 118, wherein the antigenic polypeptide associated with B/Austria/1359417/2021 is an HA polypeptide and comprises a sequence that is at least 85% identical to SEQ ID NO: 85.

Embodiment 120. The composition of embodiment 118 or 119, wherein the antigenic polypeptide associated with B/Austria/1359417/2021 is an HA polypeptide and the RNA encoding the HA polypeptide comprises a nucleotide sequence that is at least 85% identical to SEQ ID NO: 87.

Embodiment 121. The composition of embodiment 114, where the B/Yamagata lineage influenza virus is B/Phuket/3073/2013.

Embodiment 122. The composition of embodiment 121, wherein the antigenic polypeptide associated with B/Phuket/3073/2013 is an HA polypeptide and comprises an amino acid sequence that is at least 85% identical to SEQ ID NO: 105.

Embodiment 123. The composition of embodiment 121 or 122, wherein the antigenic polypeptide associated with B/Phuket/3073/2013 is an HA polypeptide, and the RNA encoding the HA polypeptide comprises a nucleotide sequence that is at least 85% identical to SEQ ID NO: 107.

Embodiment 124. The composition of any one of embodiments 61-123, wherein the first infectious agent is a coronavirus.

Embodiment 125. The composition of embodiment 124, wherein the coronavirus is an alphacoronavirus, a betacoronavirus, a gammacoronavirus, or a deltacoronavirus.

Embodiment 126. The composition of embodiment 125, wherein the coronavirus is a betacoronavirus.

Embodiment 127. The composition of embodiment 126, wherein the betacoronavirus is a sarbecovirus, a merbecovirus, an embecorvius, a nobecovirus, or a hibecorvirus.

Embodiment 128. The composition of embodiment 127, wherein the sarbecovirus is SARS-CoV-1 or SARS-CoV-2.

Embodiment 129. The composition of embodiment 128, wherein the sarbecovirus is SARS-CoV-2.

Embodiment 130. The composition of embodiment 127, wherein the merbecovirus is MERS-COV.

Embodiment 131. The composition of embodiment 129, wherein the one or more first RNAs comprise an RNA comprising a nucleotide sequence that encodes one or more antigenic polypeptides associated with a SARS-CoV-2 variant that is prevalent or has been identified as a variant of concern in a relevant population at the time of administration.

Embodiment 132. The composition of 129, wherein the one or more first RNAs comprise an RNA comprising a nucleotide sequence that encodes one or more antigenic polypeptides associated with an Omicron SARS-CoV-2 variant (e.g., a BA.1, BA.2, BA.4/5, or XBB.1.5 variant).

Embodiment 133. The composition of embodiment 129, wherein the one or more first RNAs comprise (i) an RNA comprising a nucleotide sequence that encodes one or more antigenic polypeptides associated with a first SARS-CoV-2 strain, wherein the first SARS-CoV-2 strain is a SARS-CoV-2 ancestral strain (Wuhan strain) and (ii) an RNA comprising a nucleotide sequence that encodes one or more antigenic polypeptides associated with a second SARS-CoV-2 variant, wherein the second SARS-CoV-2 is a variant of the SARS-CoV-2 ancestral strain, and is prevalent or has been identified as a variant of concern in a relevant population at the time of administration.

Embodiment 134. The composition of embodiment 129, wherein the one or more first RNAs comprise (i) an RNA comprising a nucleotide sequence that encodes one or more antigenic polypeptides associated with a first SARS-CoV-2 variant and (ii) an RNA comprising a nucleotide sequence that encodes one or more antigenic polypeptides associated with a second SARS-CoV-2 variant, wherein the first and the second SARS-CoV-2 variant are each prevalent or have been identified as a variant of concern in a relevant population at the time of administration.

Embodiment 135. The composition of embodiment 133 or 134, wherein the second SARS-CoV-2 variant is an Omicron variant of SARS-CoV-2.

Embodiment 136. The composition of embodiment 135, wherein the Omicron variant of SARS-CoV-2 is or comprises an Omicron BA.1, BA.2, BA.4/5, or XBB.1.5 variant.

Embodiment 137. The composition of any one of embodiments 124-136, wherein the antigenic polypeptide(s) associated with the coronavirus is a Spike (S) polypeptide, or a immunogenic fragment or variant thereof.

Embodiment 138. The composition of embodiment 137, wherein the S polypeptide is a prefusion stabilized S polypeptide.

Embodiment 139. The composition of embodiment 138, wherein the prefusion stabilized S polypeptide comprises at least two proline substitutions.

Embodiment 140. The composition of embodiment 139, wherein the two proline substitutions comprises proline residues at positions corresponding to residues 986 and 987 of SEQ ID NO: 1.

Embodiment 141. The composition of any one of embodiments 138-140, wherein the prefusion stabilized S polypeptide comprises at least six proline substitutions.

Embodiment 142. The composition of embodiment 141, wherein four of the at least six proline substitutions comprises proline residues at positions corresponding to residues 817, 892, 899, and 942 of SEQ ID NO: 1.

Embodiment 143. The composition of 132, wherein the RNA encoding one or more antigenic polypeptides associated with an Omicron SARS-CoV-2 variant encodes an S protein associated with an XBB.1.5 strain and comprising an amino acid sequence that is at least 85% identical to SEQ ID NO: 129.

Embodiment 144. The composition of any one of embodiments 133-142, wherein the RNA encoding one or more antigenic polypeptides associated with a SARS-CoV-2 ancestral strain encodes an S protein associated with a Wuhan strain and comprising an amino acid sequence that is at least 85% identical to SEQ ID NO: 7.

Embodiment 145. The composition of embodiment 144, wherein the RNA encoding SEQ ID NO: 7 comprises a nucleotide sequence that is at least 85% identical to SEQ ID NO: 9.

Embodiment 146. The composition of any one of embodiments 133-145, wherein the RNA encoding one or more antigenic polypeptides associated with a second SARS-CoV-2 variant encodes an S protein associated with a BA.4/5 variant, and comprises an amino acid sequence that is at least 85% identical to SEQ ID NO: 69.

Embodiment 147. The composition of embodiment 146, wherein the RNA encoding SEQ ID NO: 69 comprises a nucleotide sequence that is at least 85% identical to SEQ ID NO: 70.

Embodiment 148. The composition of any one of embodiments 61-147, wherein:
the one or more first RNAs comprise:
   (a) an RNA comprising a nucleotide sequence that encodes a SARS-CoV-2 Spike (S) polypeptide from an Omicron variant of SARS-CoV-2 (e.g., a BA.1, BA.2, BA.4/5, or XBB.1.5 variant); or
   (b) an RNA comprising a nucleotide sequence that encodes a SARS-CoV-2 Spike (S) polypeptide from a SARS-CoV-2 ancestral strain (Wuhan strain) and an RNA comprising a nucleotide sequence that encodes a SARS-CoV-2 Spike (S) polypeptide from an Omicron variant of SARS-CoV-2 (e.g., a BA.1, BA.2, BA.4/5, or XBB.1.5 variant); and
wherein the one or more second RNAs comprise: (i) an RNA comprising a nucleotide sequence that encodes an HA polypeptide from an influenza A/H1N1 virus, (ii) an RNA comprising a nucleotide sequence that encodes an HA polypeptide from an influenza A/H3N2 virus, (iii) an RNA comprising a nucleotide sequence that encodes an HA polypeptide from an influenza B/Victoria lineage virus, and (iv) an RNA comprising a nucleotide sequence that encodes an HA polypeptide from an influenza B/Yamagata virus.

Embodiment 149. The composition of embodiment 148, where the H1N1 virus is A/Wisconsin/588/2019.

Embodiment 150. The composition of embodiment 149, wherein the HA polypeptide associated with A/Wisconsin/588/2019 comprises a sequence that is at least 85% identical to SEQ ID NO: 90.

Embodiment 151. The composition of embodiment 149 or 150, wherein the RNA comprising a nucleotide sequence encoding an HA polypeptide associated with A/Wisconsin/588/2019 comprises a sequence that is at least 85% identical to SEQ ID NO: 92.

Embodiment 152. The composition of any one of embodiments 148-151, where the H3N2 virus is A/Cambodia/e0826360/2020.

Embodiment 153. The composition of embodiment 152, wherein the HA polypeptide associated with A/Cambodia/e0826360/2020 comprises a sequence that is at least 85% identical to SEQ ID NO: 95.

Embodiment 154. The composition of embodiment 152 or 153, wherein the first RNA comprising a sequence encoding an HA polypeptide associated with A/Cambodia/e0826360/2020 comprises a sequence that is at least 85% identical to SEQ ID NO: 97.

Embodiment 155. The composition of any one of embodiments 148-154, where the B/Victoria lineage influenza virus is B/Washington/02/2019.

Embodiment 156. The composition of embodiment 155, wherein the HA polypeptide associated with B/Washington/02/2019 comprises a sequence that is at least 85% identical to SEQ ID NO: 100.

Embodiment 157. The composition of embodiment 155 or 156, wherein the RNA comprising a nucleotide sequence encoding an HA polypeptide associated with B/Washington/02/2019 comprises a sequence that is at least 85% identical to SEQ ID NO: 102.

Embodiment 158. The composition of any one of embodiments 148-157, where the B/Yamagata lineage influenza virus is B/Phuket/3073/2013.

Embodiment 159. The composition of embodiment 158, wherein the HA polypeptide associated with B/Phuket/3073/2013 comprises a sequence that is at least 85% identical to SEQ ID NO: 105.

Embodiment 160. The composition of embodiment 158 or 159, wherein the RNA comprising a sequence encoding an HA polypeptide associated with B/Phuket/3073/2013 comprises a nucleotide sequence that is at least 85% identical to SEQ ID NO: 107.

Embodiment 161. The composition of any one of embodiments 148-160, wherein the S polypeptide associated with a Wuhan strain comprises a sequence that is at least 85% identical to SEQ ID NO: 7.

Embodiment 162. The composition of any one of embodiments 148-161, wherein the RNA comprising a nucleotide sequence encoding an S polypeptide associated with a Wuhan strain comprises a sequence that is at least 85% identical to SEQ ID NO: 70.

Embodiment 163. The composition of any one of embodiments 148-162, wherein the Omicron variant is a BA.4/5 variant.

Embodiment 164. The composition of embodiment 163, wherein the S polypeptide associated with the BA.4/5 Omicron variant comprises a sequence that is at least 85% identical to SEQ ID NO: 69.

Embodiment 165. The composition of embodiment 163 or 164, wherein the RNA comprising a sequence encoding an S polypeptide associated with a BA.4/5 Omicron variant comprises a sequence that is at least 85% identical to SEQ ID NO: 70.

Embodiment 166. The composition of any one of embodiments 148-160, wherein the Omicron variant is an XBB.1.5 variant.

Embodiment 167. The composition of embodiment 166, wherein the S polypeptide associated with the XBB.1.5 Omicron variant comprises a sequence that is at least 85% identical to SEQ ID NO: 129.

Embodiment 168. The composition of embodiment 166 or 167, wherein the RNA comprising a sequence encoding an S polypeptide associated with an XBB.1.5 Omicron variant comprises a sequence that is at least 85% identical to SEQ ID NO: 130.

Embodiment 169. The composition of any one of embodiments 47-60 and 62-77, wherein at least one of the non-coding elements is or comprises:
(i) a 5′-UTR sequence that is or comprises a modified human alpha-globin 5′-UTR;
(ii) a 3′-UTR sequence that is or comprises a first sequence from the amino terminal enhancer of split (AES) messenger RNA and a second sequence from the mitochondrial encoded 12S ribosomal RNA;
(iii) a polyA sequence comprising at least 100 A nucleotides, wherein the first RNA and the second RNA each do not comprise a stretch of at least 30 contiguous C nucleotides between the 3′ UTR and the polyA sequence;
(iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
(v) a 5′ cap comprising a Cap1 structure and a cap proximal sequence comprising positions +1, +2, +3, +4, and +5 of the RNA; wherein:
   (a) the Cap1 structure comprises m7(3′OMeG)(5′)ppp(5′)(2′OMeA₁)pG₂, wherein A₁ is position +1 of the RNA, and G₂ is position +2 of the RNA; and
   (b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

Embodiment 170. The composition of any one of embodiments 47-169, wherein at least one of the same non-coding elements is or comprises:
(i) a 5′-UTR sequence that is or comprises a modified human alpha-globin 5′-UTR;
(ii) a 3′-UTR sequence that is or comprises a first sequence from the amino terminal enhancer of split (AES) messenger RNA and a second sequence from the mitochondrial encoded 12S ribosomal RNA;
(iii) a polyA sequence comprising at least 100 A nucleotides, wherein the first RNA and the second RNA each do not comprise a stretch of at least 30 contiguous C nucleotides between the 3′ UTR and the polyA sequence;
(iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; and
(v) a 5′ cap comprising a Cap1 structure and a cap proximal sequence comprising positions +1, +2, +3, +4, and +5 of the RNA; wherein:
   (a) the Cap1 structure comprises m7(3′OMeG)(5′)ppp(5′)(2′OMeA₁)pG₂, wherein A₁ is position +1 of the RNA, and G₂ is position +2 of the RNA; and
   (b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

Embodiment 171. The composition of any one of embodiments 1-46, wherein each RNA comprises:
(i) a 5′ cap, wherein the 5′ cap optionally comprises a cap1 structure;
(ii) a cap proximal sequence;
(iii) a 5′ UTR sequence, wherein the 5′ UTR is optionally a modified human alpha-globin 5′-UTR;
(iv) a 3′ UTR sequence, wherein the 3′ UTR sequence optionally comprises a first sequence from the amino terminal enhancer of split (AES) messenger RNA and a second sequence from the mitochondrial encoded 12S ribosomal RNA; and/or
(v) a polyA sequence, wherein the polyA sequence optionally comprises 30 adenine nucleotides (SEQ ID NO: 174) followed by 70 adenine nucleotides (SEQ ID NO: 175), wherein the 30 adenine nucleotides (SEQ ID NO: 174) and the 70 adenine nucleotides (SEQ ID NO: 175) are separated by a linker sequence.

Embodiment 172. The composition of any one of embodiments 47-171, wherein the 5′ cap, cap proximal sequence, 5′ UTR sequence, 3′ UTR sequence, and polyA sequence are in 5′ to 3′ order.

Embodiment 173. The composition any one of embodiments 1-172, wherein each RNA comprises a 5′-cap that is or comprises m₂^{7,3′-O}Gppp(m₁^{2′-O})ApG.

Embodiment 174. The composition of any one of embodiments 47-169 and 171-173, wherein the 5′ UTR comprises or consists of a human alpha-globin 5′-UTR.

Embodiment 175. The composition of embodiment 174, wherein the human alpha-globin 5′-UTR comprises SEQ ID NO: 12.

Embodiment 176. The composition of any one of embodiments 47-169, and 171-175, wherein the 3′ UTR comprises or consists of a first sequence from the amino terminal enhancer of split (AES) messenger RNA and a second sequence from the mitochondrial encoded 12S ribosomal RNA

Embodiment 177. The composition of embodiment 176, wherein the 3′ UTR comprises or consists of a sequence according to SEQ ID NO: 13.

Embodiment 178. The composition of any one of embodiments 47-177, wherein the polyA tail sequence is an interrupted polyA tail sequence.

Embodiment 179. The composition of embodiment 178, wherein the interrupted polyA tail sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence.

Embodiment 180. The composition of embodiment 179, wherein the interrupted polyA tail sequence comprises or consists of a ribonucleic acid sequence according to SEQ ID NO: 14.

Embodiment 181. The composition of any one of embodiments 47-180, wherein the sequence at the 3′ end of the 3′UTR (e.g., the sequence immediately adjacent to the polyA tail sequence) is CUXGAGCUAGC (SEQ ID NO: 176), wherein X is G, C, A, or U.

Embodiment 182. The composition of any one of embodiments 47-181, wherein the sequence at the 3′ end of the 3′UTR (e.g., the sequence immediately adjacent to the polyA tail) is GAGACCUGGUCCAGAGUCGCUAGCCGCGUCGCU (SEQ ID NO: 177).

Embodiment 183. The composition of any one of embodiments 47-182, wherein the sequence at the 3′ end of the 3′UTR (e.g., the sequence immediately adjacent to a sequence encoding an antigenic polypeptide) is CUCGAG or GGAUCCGAU.

Embodiment 184. The composition of any one of embodiments 1-183, wherein each RNA in the composition includes modified uridines in place of all uridines.

Embodiment 185. The composition of embodiment 184, wherein the modified uridines are each N1-methyl-pseudouridine.

Embodiment 186. The composition of any one of embodiments 47 to 185, wherein the first RNAs and second RNAs are present in a mass ratio of 1:5 to 5:1, 1:4 to 4:1, 1:3 to 3:1, or 1:2 to 2:1.

Embodiment 187. The composition of any one of embodiments 1 to 186, wherein each of the RNAs in the composition is formulated in nanoparticles.

Embodiment 188. The composition of any one of embodiments 47 to 187, wherein all of the first RNAs are co-formulated together in the same population of nanoparticles and all of the second RNAs are co-formulated together in the same population of nanoparticles, and wherein the first RNAs and the second RNAs are formulated in separate populations of nanoparticles.

Embodiment 189. The composition of any one of embodiments 47 to 188, wherein the first RNAs and the second RNAs are all co-formulated together in the same population of nanoparticles.

Embodiment 190. The composition of any one embodiments 1-60 and 96-190, wherein:
(a) each RNA encoding an antigenic polypeptide of an influenza A virus is coformulated in a first population of nanoparticles, and each RNA encoding an antigenic polypeptide of an influenza B virus is coformulated in a second population of nanoparticles;
(b) each RNA encoding an antigenic polypeptide of an influenza virus is formulated in a separate population of nanoparticles; or
(c) each RNA encoding an antigenic polypeptide of an influenza A virus is coformulated in a first population of nanoparticles, and each RNA encoding an antigenic polypeptide of an influenza B virus is formulated in separate nanoparticles.

Embodiment 191. The composition of any one of embodiments 187-190, wherein the nanoparticles comprise lipid nanoparticles, polyplexes (PLX), lipidated polyplexes (LPLX), liposomes, or polysaccharide nanoparticles.

Embodiment 192. The composition of embodiment 191, wherein the nanoparticles comprise lipid nanoparticles.

Embodiment 193. The composition of embodiment 192, wherein the lipid nanoparticles each comprise: a cationically ionizable lipid; and one or more neutral lipids, and a polymer-conjugated lipid.

Embodiment 194. The composition of embodiment 193, wherein the polymer-conjugated lipid comprises a PEG-conjugated lipid.

Embodiment 195. The composition of any one of embodiments 188-194, wherein the nanoparticles have an average diameter of about 50-150 nm.

Embodiment 196. The composition of any one of embodiments 1-195, further comprising:
(a) one or more third RNAs, each comprising a nucleotide sequence that encodes one or more antigenic polypeptides associated with a third infectious agent that is different from the first infectious agent and the second infectious agent; or
(b) one or more polypeptides of a third infectious agent.

Embodiment 197. The composition of embodiment 196, wherein the third infectious agent is a respiratory virus (e.g., a respiratory virus that is not a SARS-CoV-2 virus or an influenza virus).

Embodiment 198. The composition of embodiment 197, wherein the third infectious agent is a respiratory syncytial virus (RSV).

Embodiment 199. The composition of embodiment 198, wherein:
(i) the composition comprises one or more RNAs, each encoding an RSV polypeptide; or
(ii) the composition comprises one or more RSV polypeptides.

Embodiment 200. The composition of embodiment 199, wherein:
(i) the composition comprises one or more RNAs, each encoding an RSV F protein, a variant thereof, or an immunogenic fragment of an RSV F protein or a variant thereof; or
(ii) the composition comprises one or more RSV F proteins, an immunogenic variant thereof, or an immunogenic fragment of an RSV F protein or a variant thereof.

Embodiment 201. The composition of embodiment 199 or 200, wherein:
(i) the composition comprises one or more RNAs, each encoding a polypeptide of an RSV subtype A virus (e.g., an F protein of an RSV subtype A virus, a variant thereof, or an immunogenic fragment of an F protein of an RSV subtype B virus or a variant thereof), and one or more RNAs, each encoding a polypeptide of an RSV subtype B virus (e.g., an F protein of an RSV subtype B virus, a variant thereof, or an immunogenic fragment of an F protein of an RSV subtype B virus or a variant thereof); or
(ii) the composition comprises one or more polypeptides of an RSV subtype A virus (e.g., an F protein, a variant thereof, or an immunogenic fragment of an F protein or a variant thereof) and one or more polypeptides of an RSV subtype B virus (e.g., an F protein, a variant thereof, or an immunogenic fragment of an F protein or a variant thereof).

Embodiment 202. The composition of embodiment 200 or 201, wherein the RSV F protein, the variant, or the immunogenic fragment is stabilized in a prefusion confirmation.

Embodiment 203. The composition of any one of 196-202, comprising Arexvy™ or Abrysvo™.

Embodiment 204.A pharmaceutical composition comprising the composition of any one of embodiments 1-203 and at least one pharmaceutically acceptable excipient.

Embodiment 205. The pharmaceutical composition of embodiment 204, comprising a cryoprotectant, optionally wherein the cryoprotectant is or comprises sucrose.

Embodiment 206. The pharmaceutical composition of embodiment 204 or 205, wherein the pharmaceutical comprises an aqueous buffered solution, optionally wherein the aqueous buffered solution comprises one or more of Tris base, Tris HCl, NaCl, KCl, Na₂HPO₄, and KH₂PO₄.

Embodiment 207. The pharmaceutical composition of any one of embodiments 204-206, formulated to provide a dose of 100 μg or less of total RNA.

Embodiment 208. The pharmaceutical composition of embodiment 207, formulated to provide a dose of 90 μg of total RNA.

Embodiment 209. The pharmaceutical composition of embodiment 207, formulated to provide a dose of 60 μg of total RNA.

Embodiment 210. The pharmaceutical composition of embodiment 208, formulated to provide a dose of 30 μg of one or more first RNAs and a dose of 60 μg of one or more second RNAs.

Embodiment 211. The pharmaceutical composition of embodiment 208, formulated to provide a dose of 60 μg of one or more first RNAs and a dose of 30 μg of one or more second RNAs.

Embodiment 212. The pharmaceutical composition of embodiment 209, formulated to provide a dose of 30 μg of one or more first RNAs and a dose of 30 μg of one or more second RNAs.

Embodiment 213. The pharmaceutical composition of embodiment 208 or 210, comprising four second RNAs, each comprising a nucleotide sequence that encodes an antigenic polypeptide associated with a different influenza virus, and wherein the pharmaceutical composition is formulated to provide a dose of 15 μg of each second RNA.

Embodiment 214. The pharmaceutical composition of embodiment 209 or 211, comprising four second RNAs, each comprising a nucleotide sequence that encodes an antigenic polypeptide associated with a different influenza virus, and wherein the pharmaceutical composition is formulated to provide a dose of 7.5 μg of each second RNA.

Embodiment 215. The pharmaceutical composition of any one of embodiments 208-210 and 212-214, comprising two first RNAs, each comprising a nucleotide sequence that encodes an antigenic polypeptide associated with a different coronavirus virus, and wherein the pharmaceutical composition is formulated to provide a dose of 15 μg of each first RNA.

Embodiment 216. The pharmaceutical composition of embodiment 211, comprising two first RNAs, each comprising a nucleotide sequence that encodes an antigenic polypeptide associated with a different coronavirus virus, and wherein the pharmaceutical composition is formulated to provide a dose of 30 μg of each first RNA.

Embodiment 217.A method comprising administering to a subject one or more doses of the composition of any one of embodiments 1-213 or one or more doses of the pharmaceutical composition of any one of embodiments 204-216.

Embodiment 218. The method of embodiment 217, wherein the method is a method of treating a coronavirus disease and influenza disease.

Embodiment 219. The method of embodiment 217, wherein the method is a method of (i) preventing a coronavirus disease and an influenza disease or (ii) inducing an immune response against a coronavirus and/or an influenza virus.

Embodiment 220. The method of any one of embodiments 217-219, wherein each the one or more doses of the composition or each of the one or more doses of the pharmaceutical composition is co-administered with a vaccine against a third infectious agent.

Embodiment 221. The method of embodiment 220, wherein the third infectious agent is a virus that can cause a respiratory disease.

Embodiment 222. The method of embodiment 220, wherein the third infectious agent is RSV.

Embodiment 223. The method of embodiment 222, wherein the vaccine against the third infectious agent is Arexvy™ or Abrysvo™.

Embodiment 224. The method of any one of embodiments 220-223, wherein:
the vaccine against the third infectious agent is mixed with the one or more doses of the composition or the one or more doses of the pharmaceutical compositions immediately before administering to the subject; or
the vaccine against the third infectious agent is administered separately from the one or more doses of the composition or the one or more doses of the pharmaceutical compositions (e.g., wherein the vaccine against the third infectious agent and the one or more doses of the composition or the one or more doses of the pharmaceutical composition are administered to the subject at separate injection sites (e.g., on opposite arms)).

Embodiment 225. The composition of any one of embodiments 1-203 or the pharmaceutical composition of any one of embodiments 204-216, for use in the treatment of a coronavirus disease and an influenza disease, wherein the use comprises administering one or more doses of the composition or pharmaceutical composition to a subject.

Embodiment 226. The composition of any one of embodiments 1-203 or the pharmaceutical composition of any one of embodiments 204-216, for use in:
(a) the prevention of a coronavirus disease and an influenza disease, or
(b) inducing an immune response against a coronavirus and an influenza virus,
wherein the use comprises administering one or more doses of the composition or pharmaceutical composition to a subject.

Embodiment 227. The method of any one of embodiments 217-224, or the composition or pharmaceutical composition for use of embodiment 225 or 226, wherein the method or the use comprises administering two or more doses of the composition or pharmaceutical composition to the subject.

Embodiment 228. The method or composition or pharmaceutical composition for use of embodiment 227, wherein the two doses are administered at least about 21 days apart.

Embodiment 229. The method of any one of embodiments 217-224, or the composition or pharmaceutical composition for use of embodiment 227 or 228, wherein the method or the use comprises administering three or more doses of the composition or pharmaceutical composition to the subject.

Embodiment 230. The method of any one of embodiments 217-224, or the composition or pharmaceutical composition for use of embodiment 225 or 226, wherein the subject has previously been exposed to a coronavirus and/or an influenza virus (e.g., by vaccination and/or by infection).

Embodiment 231. The method or composition or pharmaceutical composition for use of any one of embodiments 217-230, wherein the method or use induces an immune response in the subject against one or coronaviruses and one or more influenza viruses.

Embodiment 232. The method or composition or pharmaceutical composition for use of embodiment 231, wherein the immune response comprises a B-cell response.

Embodiment 233. The method or composition or pharmaceutical composition for use of embodiment 232, wherein the B cell response comprises production of antibodies directed against the one or more antigens.

Embodiment 234. The method or composition or pharmaceutical composition for use of embodiment 232 or 233, wherein the immune response comprises a T cell response.

Embodiment 235. The method or composition or pharmaceutical composition for use of embodiment 234, wherein the T-cell response is or comprises a CD4+ T cell response.

Embodiment 236. The method or composition or pharmaceutical composition for use of embodiment 234 or 235, wherein the T-cell response is or comprises a CD8+ T cell response.

Embodiment 237. Use of the composition of any one of embodiments 1-203, or the pharmaceutical composition of any one of embodiments 204-216 in the treatment of a coronavirus disease and an influenza disease in a subject.

Embodiment 238. Use of the composition of any one of embodiments 1-203 or the pharmaceutical composition of any one of embodiments 204-217 in the prevention of a coronavirus disease and an influenza disease in a subject.

Embodiment 239. Use of the composition of any one of embodiments 1-203 or the pharmaceutical composition of any one of embodiments 204-217 in inducing an immune response in a subject against one or more coronaviruses and one or more influenza viruses.

Embodiment 240. A method for inducing an immune response against a first infectious agent and a second infectious agent, wherein the method comprises administering
(i) a first nanoparticle (e.g., LNP) formulated RNA comprising a nucleotide sequence encoding an antigenic polypeptide associated with a first infectious agent; and
(ii) a second nanoparticle (e.g., LNP) formulated RNA comprising a nucleotide sequence encoding an antigenic polypeptide associated with a second infectious agent,
wherein the immune response induced against each of the first and the second infectious agents is greater than the immune response induced when the nanoparticles are administered separately.

Embodiment 241. A method for reducing the amount of a first nanoparticle (e.g. LNP) formulated RNA required to produce an immune response against a first infectious agent, wherein the RNA of the first nanoparticle-formulated RNA comprises a nucleotide sequence encoding one or more antigenic polypeptides associated with a first infectious agent,
wherein the method comprises co-administering a second nanoparticle (e.g., LNP)-formulated RNA comprising a nucleotide sequence encoding one or more antigenic polypeptides associated with a second infectious agent, and
wherein the first infectious agent differs from the second infectious agent.

Embodiment 242.A composition comprising:
one or more RNAs, each encoding a polypeptide of a first infectious agent; and
one or more polypeptides of a second infectious agent.

Embodiment 243. The composition of embodiment 242, wherein the first infectious agent is a coronavirus.

Embodiment 244. The composition of embodiment 243, wherein the coronavirus is a SARS-CoV-2 virus.

Embodiment 245. The composition of embodiment 244, wherein the one or more RNAs each encode a SARS-CoV-2 S protein, a variant thereof, or an immunogenic fragment of a SARS-CoV-2 S protein or variant thereof.

Embodiment 246. The composition of embodiment 245, comprising one or more RNAs, each encoding a SARS-CoV-2 S protein, a variant thereof, or an immunogenic fragment of a SARS-CoV-2 S protein or variant thereof of a Wuhan strain or a SARS-CoV-2 variant (e.g., an Omicron variant (e.g., an Omicron BA.1, BA.2, BA.4/5, or an XBB.1.5 variant (e.g., an RNA described herein))).

Embodiment 247. The composition of any one of embodiments 242-246, wherein the second infectious agent is an influenza virus.

Embodiment 248. The composition of embodiment 247, wherein the composition comprises one or more polypeptides of one or more influenza viruses (e.g., one or more polypeptides of two or more influenza virus strains (e.g., one or more polypeptides of four or more influenza virus strains that are prevalent or which have been predicted to be prevalent in a relevant jurisdiction)).

Embodiment 249. The composition of embodiment 247 or 248, comprising a commercially available influenza virus (e.g., a recombinant commercially available influenza virus described herein).

Embodiment 250. The composition of embodiment 249, wherein the commercially available influenza virus is Flublok.

Embodiment 251. The composition of any one of embodiments 241-246, wherein the second infectious agent is an RSV.

Embodiment 252. The composition of embodiment 251, comprising one or more polypeptides associated with a first RSV subtype and one or more polypeptides of a second RSV subtype.

Embodiment 253. The composition of embodiment 251 or 252, wherein the polypeptide of the second infectious agent is an RSV F protein, a variant thereof, or an immunogenic fragment of either of an RSV F protein or a variant thereof.

Embodiment 254. The composition of embodiment 253, wherein the RSV F protein, the variant, or the immunogenic fragment comprises one or more mutations that stabilize a prefusion confirmation of the F protein.

Embodiment 255. The composition of embodiment 254, comprising Arexvy™ or ABRYSVO™.

Embodiment 256. The composition of any one of embodiments 241-255, further comprising one or more polypeptides of a third infectious agent.

Embodiment 257. The composition of embodiment 256, comprising:
one or more RNAs, each encoding one or more polypeptides of a coronavirus (e.g., a SARS-CoV-2 S protein, a variant thereof, or an immunogenic fragment of either of the foregoing);
one or more polypeptides of one or more influenza viruses; and
one or more polypeptides of one or more RSVs.

Embodiment 258. The composition of embodiment 257, comprising:
an RNA encoding a SARS-CoV-2 S protein of an Omicron variant (e.g., an RNA encoding an S protein of an Omicron BA.1, BA.4/5, or XBB.1.5 variant described herein);
a recombinant influenza vaccine (e.g., as described herein (e.g., a FluBlok vaccine)); and
an RSV vaccine comprising a prefusion-stabilized F protein or an immunogenic fragment thereof (e.g., an RSV vaccine described herein (e.g., Arexvy™ or ABRYSVO™)).

Embodiment 259.A combination comprising a SARS-CoV-2 vaccine comprising one or more mRNAs encoding a prefusion stabilized SARS-CoV-2 spike protein or a variant thereof; and
(a) an influenza vaccine comprising (i) one or more mRNAs encoding an HA protein of an influenza virus, or (ii) one or HA polypeptides, and/or
(b) an RSV vaccine comprising one or more prefusion stabilized RSV F proteins or an immunogenic fragment thereof.

Embodiment 260. The combination of embodiment 259, wherein the one or more mRNAs encoding a prefusion stabilized SARS-CoV-2 spike protein or a variant thereof is formulated as an LNP.

Embodiment 261. The combination of embodiment 259 or 260, wherein the one or more mRNAs encoding an HA protein of an influenza virus is formulated as an LNP.

Embodiment 262. The combination of any one of embodiments 259-261, wherein the (1) SARS-CoV-2 vaccine and the (2) influenza vaccine or RSV vaccine are provided in separate containers (e.g., vials or syringes).

Embodiment 263. The combination of any one of embodiments 259-261, wherein the (1) SARS-CoV-2 vaccine and the (2) influenza vaccine or RSV vaccine are provided in a single containers (e.g., vial or syringe).

Embodiment 264. The combination of any one of embodiments 259-263, comprising the SARS-CoV-2 vaccine, the influenza vaccine, and the RSV vaccine.

Embodiment 265. The combination of embodiment 264, wherein the SARS-CoV-2 vaccine, the influenza vaccine, and the RSV vaccine are provided in a single container (e.g., a vial or syringe).

Embodiment 266. The combination of embodiment 264, wherein the SARS-CoV-2 vaccine, the influenza vaccine, and the RSV vaccine are provided in separate containers (e.g., separate vials and/or syringes).

Embodiment 267. The combination of embodiment 264, wherein:
(a) the SARS-CoV-2 vaccine and the influenza vaccine are provided in a single container, and the RSV vaccine is provided in a separate container; or
(b) the SARS-CoV-2 vaccine and RSV vaccine are provided in a single container, and the influenza vaccine is provided in a separate container.

Embodiment 268. The combination any one of embodiments 259-267, wherein the SARS-CoV-2 vaccine is BNT162b2 (e.g., a monovalent or bivalent vaccine described herein).

Embodiment 269. The combination any one of embodiments 259-268, wherein the influenza vaccine is a recombinant influenza vaccine (e.g., as described herein (e.g., a FluBlok vaccine)); or comprises an inactivated influenza virus (e.g., Fluzone).

Embodiment 270. The combination of any one of embodiments 259-269, wherein the RSV vaccine comprises a prefusion-stabilized F protein or an immunogenic fragment thereof (e.g., an RSV vaccine described herein (e.g., Arexvy™ or ABRYSVO™)).

Citation of documents and studies referenced herein is not intended as an admission that any of the foregoing is pertinent prior art. All statements as to the contents of these documents are based on the information available to the applicants and do not constitute any admission as to the correctness of the contents of these documents.

The contents of each of the references cited herein are incorporated by reference in their entirety for purposes described herein.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Influenza RNA Vaccine Clinical Trial

The present Example describes an exemplary clinical trial protocol that is currently in progress, and which investigates an RNA influenza vaccine (in particular a modRNA, tetravalent modRNA vaccine). The described clinical trial protocol comprises a first Phase 1/2 randomized, observer-blinded (Sponsor-unblinded) study, which is ongoing and which is designed to evaluate the safety, tolerability, and immunogenicity of a modRNA vaccine against influenza in healthy individuals.

Following completion of the Phase 1/2 study, a Phase 3 will then be performed.

### Phase 1/2 Study

A Phase 1/2 study to describe the safety, tolerability, and immunogenicity of a modRNA vaccine against influenza in healthy individuals was initiated in September 2021 in participants 65 through 85 years of age. This study is being conducted across

2 substudies—Substudy A and Substudy B. Substudy A describes the safety and immunogenicity of mIRV (monovalent influenza RNA Vaccine) A or B at 4 dose levels, bIRV (binovalent influenza RNA Vaccine) encoding both A and B strains in 4 dose-level combinations, and qIRV (quadravalent influenza RNA Vaccine) encoding 2 A strains and 2 B strains at a dose level of 7.5 μg per strain. Additionally, Substudy A describes:
The immune response elicited by licensed QIV following prior receipt of a modRNA vaccine, to assess potential priming of the immune response, and
The immune response elicited by mIRV A or B following prior receipt of licensed QIV, to assess if the immune response following QIV may be enhanced.

Substudy B describes the safety and immunogenicity of the following vaccination schedules:

In participants 65 through 85 years of age:
2 Doses of qIRV encoding 2 A strains and 2 B strains at a dose level of 7.5 μg per strain, administered 21 days apart.
2 Doses of licensed QIV, administered 21 days apart (as a control group).
A dose of licensed QIV following by a dose of bIRV encoding 2 A strains at a dose level of either 15 or 30 μg per strain, administered 21 days apart.
A dose of licensed QIV administered concurrently in the opposite arm with bIRV encoding 2 A strains at a dose level of either 15 or 30 μg per strain.
A dose of bIRV encoding 2 A strains at a dose level of 15 μg per strain administered concurrently in the opposite arm with bIRV encoding 2 B strains at a dose level of 15 μg per strain.
A dose of qIRV encoding 2 A strains and 2 B strains at the following dose-level combinations:
   15 μg per strain
   7.5 μg per A strain, and 22.5 μg per B strain
   7.5 μg per A strain, and 37.5 μg per B strain
A dose of licensed QIV (as a control group).

In participants 18 through 64 years of age:
A dose of qIRV encoding 2 A strains and 2 B strains at a dose level of 7.5 μg per strain.
A dose of qIRV encoding 2 A strains and 2 B strains at a dose level of 15 μg per strain.

Preliminary results indicated that 30 μg and 60 μg qIRV were well tolerated with mainly mild to moderate local reactions and systemic events consistent with rates observed with BNT162b2 and support the initiation of a Phase 3 study. Immunogenicity data available to date indicated that:
For A/Wisconsin and A/Cambodia, the proportion of participants achieving seroconversion 4 weeks following either 1 or 2 doses of qIRV, at a total dose level of 30 μg per dose, was higher than the proportion of participants achieving seroconversion following either 1 or 2 doses of QIV. Additionally, 4 weeks following QIV administered with bIRV encoding 2 A strains at a total dose of 30 or 60 μg (15 or 30 μg per strain), either concurrently or 21 days apart, also appeared to be higher than the proportion of participants achieving seroconversion following either 1 or 2 doses of QIV.
For B/Phuket and B/Washington, the proportion of participants achieving seroconversion 4 weeks following study intervention in all groups, including 1- and 2-dose QIV recipients, was lower compared to the proportion of participants achieving seroconversion against A/Wisconsin and A/Cambodia.

Based on the safety and immunogenicity data from the phase 1/2 study, 1 dose of qIRV encoding 2 A strains and 2 B strains was selected for study in Phase 3.

### Phase 3 Study

Based on Phase 1/2 safety and immunogenicity data collected, the Phase 3 trial is designed to test a quadrivalent influenza vaccine at a 60-μg dose (comprising 15-μg of RNA for two type A and two type B strains) for participants ≥65 years of age. Phase 1/2 data analyses for adults 18 to 64 years of age are reviewed to determine if the Phase 3 dose level in this age group can be 60 μg or 30-μg (including 7.5-μg each of the 2 type A and 2 type B strains). Table 35, below, summarizes doses being investigated in the Phase 3 study.

TABLE 35
modRNA Influenza Vaccine Dose Levels
and Regimens by Age Group

| Age Group | Dose Level | Vaccine Regimen |
|---|---|---|
| ≥65 years of age | 60-μg | qIRV single dose intended |
| | | as annual booster |
| 18 to 64 years | 30-μg or 60-μg | qIRV single dose intended |
| of age | | as annual booster |

The Phase 3 study is a randomized, observer-blinded, multicenter study to evaluate efficacy, safety, tolerability, and immunogenicity of a quadravalent influenza RNA vaccine (qIRV, the composition of which is selected based on prior Phase 1/2 evaluations) in individuals ≥18 years of age. The study randomizes approximately 25,000 participants at a 1:1 ratio to receive a single dose of qIRV or an approved quadravalent influenza vaccine (QIV, administered at an approved dose). Enrollment is stratified by age to include approximately 13,600 participants ≥65 years of age and approximately 11,400 participants 18 to 64 years of age.

A 60-μg dose was selected for Phase 3 study in participants ≥65 years of age. The dose to be tested for adults 18 to 64 years of age is either 30 μg or 60-μg, depending on results from an ongoing Phase 1/2 data for this age group.

Planned clinical assessments in the Phase 3 study include efficacy (e.g., demonstration of superiority of qIRV versus QIV based on laboratory-confirmed influenza illness incidences), immunogenicity (e.g., demonstration of noninferiority and/or superiority of qIRV versus QIV based on HAI titers), and safety evaluations (e.g., reactogenicity and adverse event [AE] reporting).

### Example 2: Overview of Nonclinical Testing Strategy

The primary pharmacology of influenza modRNA and COVID-19 modRNA vaccines described herein have been evaluated independently, e.g., as described in the previous Examples. These studies demonstrated in vitro expression of vaccine antigens from modRNA constructs in cultured cells, in vivo immunogenicity in several animal species, and a SARS-CoV-2 challenge study in nonhuman primates to assess protection against infection and to demonstrate lack of disease enhancement.

ADME characterization included non-GLP in vivo testing of an LNP-formulated modRNA encoding luciferase to examine biodistribution in BALB/c mice and Wistar Han rats after IM injection. Potential biodistribution of an influenza modRNA-bivalent COVID-19 vaccine was assessed using luciferase expression as a surrogate reporter or radiolabeled [3H]-CHE, a nonexchangeable, nonmetabolizable lipid marker.

Toxicity of an influenza modRNA-LNP vaccine and COVID-19 vaccine have been well-characterized separately, including in multiple GLP-compliant repeat-dose toxicity studies and GLP-compliant developmental and reproductive toxicity (DART) studies performed in Wistar Han rats. An intramuscular (IM) route of administration was selected for these studies. Rationale for an IM route of administration and species selection (rats) for nonclinical safety evaluation of both influenza modRNA-LNP vaccine and COVID-19 vaccines is consistent with WHO guidance (World Health Organization. Annex 1. WHO Guidelines on the non-clinical evaluation of vaccines. WHO Technical Report Series No. 927. Geneva, Switzerland; World Health Organization; 2005:31-6.). On the basis of this previously collected toxicity data, doses of a combination vaccine is clinically supported up to at least 100 μg of total RNA.

### Pharmacology

### Brief Summary

Primary pharmacology of influenza modRNA and COVID-19 modRNA vaccines have been evaluated independently in their respective vaccine programs. These studies demonstrated in vitro expression of vaccine antigen from modRNA constructs in cultured cells, in vivo immunogenicity of modRNA-LNP vaccines in different animal species, and included a SARS-CoV-2 challenge study in nonhuman primates to assess protection against infection and to demonstrate lack of disease enhancement. These in vitro and in vivo studies demonstrated that both a SARS-CoV-2 modRNA vaccine and an Influenza modRNA vaccine can induce an immune response characterized by a strong functional antibody response and a Th1-type CD4+ and an IFNγ+CD8+ T-cell response.

### Pharmacokinetics

### Brief Summary

Assessment of the ADME profile of a modRNA-LNP formulation was characterized for both a COVID-19 vaccine and an influenza vaccine. These studies included evaluating the potential biodistribution of an influenza modRNA-bivalent COVID-19 vaccine using luciferase expression as a surrogate reporter in BALB/c mice. The luciferase reporter was used as it was a readily available reporter that has been widely used to develop an understanding of protein/organ expression (Chen et al, 2020; Elia et al, 2020; Fukuchi et al, 2020; Hassett et al, 2019; Truong et al, 2019; Barry et al, 2012; Jeon et al, 2006). Mice were administered a luciferase expressing modRNA formulated like an influenza modRNA-bivalent COVID-19 vaccine, with identical lipid composition. Luciferase expression was measured in vivo following luciferin application. Luciferase expression was identified at the injection site at 6 hours after injection and was not detected after 9 days. Expression in the liver was also present to a lesser extent at 6 hours after injection and was not detected by 48 hours after injection. Distribution was also examined in male and female Wistar Han rats using LNPs with a comparable lipid composition to an influenza modRNA-bivalent COVID-19 vaccine but with a surrogate luciferase RNA and containing trace amounts of radiolabeled [3H]-CHE, a nonexchangeable, nonmetabolizable lipid marker. The greatest mean concentration of LNP was found remaining in the injection site in both sexes. Total recovery (% of injected dose) of LNP outside of the injection site was greatest in the liver and was much less in the spleen, adrenal glands, and ovaries.

### Distribution

In an in vivo study for a COVID-19 and an influenza vaccine, biodistribution was assessed using luciferase as a surrogate marker protein, with RNA encoding luciferase formulated in an LNP (e.g., using an LNP formulation disclosed herein). LNP-formulated luciferase-encoding modRNA was administered to BALB/c mice by IM injection of 1 μg each in the right and left hind leg (for a total of 2 μg). Using in vivo bioluminescence after injection of luciferin substrate, luciferase protein expression was detected at different timepoints at the site of injection and to a lesser extent, and more transiently, in the liver. Distribution to the liver is likely mediated by LNPs entering the blood stream. The luciferase expression at the injection sites dropped to background levels after 9 days. Repeat-dose toxicity studies in rats, using the BNT162 vaccines with the same modRNA-LNP platform, showed no evidence of liver injury.

Without wishing to be bound by a particular theory, biodistribution of antigen encoded by the RNA component of an influenza modRNA-bivalent COVID-19 vaccine can in part depend on LNP distribution.

### Toxicology

Briefly, repeat-dose and DART toxicity results were similar for influenza modRNA and COVID-19 vaccines. The non-clinical safety of the modRNA-LNP platform was well characterized previously in the development of COVID-19 vaccines. These previous studies evaluated up to 4 vaccine candidates in 2 GLP-compliant repeat-dose toxicity studies and a GLP compliant combined DART study. No adverse findings were observed in these studies.

Nonadverse findings were generally consistent with immune/inflammatory responses to a vaccine. The similar toxicology findings of these vaccine candidates support the platform-based safety. Indeed, in a separate program, similar nonclinical safety profile was observed with several influenza vaccine candidates using the same modRNA-LNP platform.

In the repeat-dose toxicity study, once biweekly IM administration of influenza modRNA vaccine targeting the A/Wisconsin/588/2019, A/Cambodia/e0826360/2020, B/Phuket/3073/2013, and B/Washington/02/2019 strains for a total of 2 doses up to 30 μg RNA/dose in rats was tolerated with no evidence of systemic toxicities. Similar nonadverse findings to those of BNT162 vaccine candidates (BNT162b1, BNT162b2 [V8 or V9], or BNT162b3) were noted. Expected inflammatory responses to the vaccine were observed, such as erythema and edema at the injection sites, body temperature increases, elevations in WBCs and acute phase reactants, lower A/G ratios, enlarged draining iliac lymph nodes and spleen, and microscopic inflammation at injection sites and surrounding tissues as well as increased cellularity in the draining (iliac) and inguinal lymph nodes, bone marrow, and spleen. The animals administered modRNA vaccine exhibited a hemagglutinin inhibition response to all relevant influenza strains in the vaccine.

Similarly, once weekly IM administrations of 4 BNT162 vaccine candidates (using the same modRNA-LNP platform) for a total of 3 weekly doses up to 100 μg/dose to Wistar Han rats were tolerated with no evidence of systemic toxicities. Expected inflammatory responses to the vaccine were observed, such as erythema and edema at the injection sites, body temperature increases, elevations in WBCs and acute phase reactants, increased alpha-2 macroglobulin, and lower A: G ratios, enlarged draining iliac and/or inguinal lymph nodes and spleen, and microscopic inflammation at injection sites and surrounding tissues as well as increased cellularity in the draining (iliac) and/or inguinal lymph nodes, bone marrow, and spleen. A robust SARS-CoV-2 S-specific antibody response was elicited to all BNT162 antigens.

In the DART study, IM administration of influenza modRNA vaccine at 30 μg RNA/dose before and during gestation to Wistar Han female rats was tolerated with no indication of maternal systemic toxicity. There were no influenza modRNA vaccine-related effects on fertility, gestation, or lactation in female rats or on embryo-fetal or postnatal survival, growth, or development in the F1 offspring. Vaccine-induced hemagglutinin titers to each of the 4 influenza strains were observed at all timepoints (DS 22, GD 21, and LD 21) in the F0 females and in their F1 offspring (fetuses or pups).

Similarly, IM administration of 3 BNT162 vaccine candidates to female rats twice before the start of mating and twice during gestation at the human clinical dose (30 μg RNA/dosing day) showed no effects on mating performance, fertility, or any ovarian or uterine parameters in the F0 female rats or on embryo-fetal or postnatal survival, growth, or development in the F1 offspring through the end of lactation. A SARS-CoV-2 neutralizing antibody response was confirmed in F0 female rats prior to mating, at the end of gestation, and at the end of lactation. The neutralizing antibodies were also detectable in the F1 offspring (fetuses and pups).

Combination toxicity studies with influenza modRNA vaccine and bivalent COVID-19 vaccine have not been conducted. However, findings in both studies were similar and included lack of systemic toxicity with expected inflammatory responses to the vaccine (erythema and edema at the injection sites, body temperature increases, elevations in WBCs and acute phase reactants, and lower A/G ratios). Additionally, the total RNA in the combination vaccine is no greater than the maximally tested previously (100 μg RNA). All findings from all studies for both vaccines were considered nonadverse and transient in nature. Due to similar findings among both programs, the findings would only be expected to be of greater magnitude in a combination study than those if administered alone at their individual equivalent dose.

### Target Organ Toxicity

Combination toxicity studies with influenza modRNA vaccine and bivalent COVID-19 vaccine have not been conducted. However, findings in both studies were similar and included lack of systemic toxicity with expected inflammatory responses to the vaccine.

Increased dose increased the magnitude of findings in both programs. Target organs included the injection site, immune organs (draining and regional lymph nodes, spleen, and bone marrow), and liver in rats for this modality. All findings were nonadverse and mostly reversible during the recovery period. Injection site findings included transient erythema and edema and microscopic mixed cell inflammation with edema. Immune organ effects included macroscopic enlargement of the draining and regional lymph nodes correlating microscopically to increased germinal center cellularity and increased plasma cells; increased spleen weights correlating to germinal center cellularity and increased hematopoiesis; and increased bone marrow hematopoiesis. All immune organ effects were the result of immune stimulation and/or inflammation associated with vaccine administration. Hepatocellular vacuolation was minimal and reversible without evidence of hepatocyte injury and was likely associated with the LNP uptake. There may be an increase in reactogenicity events compared to the administration of a single vaccine. Additionally, the total RNA in the combination vaccine is no greater than the maximally tested previously (100 μg RNA).

### Metabolism

Without wishing to be bound by a particular theory, protein encoded by RNA in an influenza modRNA-bivalent COVID-19 vaccine may be proteolytically degraded like other endogenous proteins. RNA is degraded by cellular RNases and subjected to nucleic acid metabolism. Nucleotide metabolism occurs continuously within the cell, with the nucleoside being degraded to waste products and excreted or recycled for nucleotide synthesis.

### Integrated Overview And Conclusions

Influenza modRNA and COVID-19 vaccines were evaluated for immunogenicity independently during development of Covid and Flu vaccines. Both vaccine candidates have been previously shown to be immunogenic in preclinical animal models. In vitro and in vivo studies established a similar mechanism-of-action by the modRNA-LNP vaccine candidates, which is to encode the vaccine antigen (influenza HA or SARS-CoV-2 Spike protein) that induces an immune response characterized by both a strong functional antibody response and a Th1-type CD4+ and an IFNγ+CD8+ T-cell response.

Biodistribution of influenza modRNA and COVID-19 vaccines was assessed using luciferase expressing modRNA, formulated similar to the influenza modRNA-bivalent COVID-19 vaccine characterized in Example (i.e., with identical lipid composition) 21 in BALB/c mice. After IM injection of the LNP-formulated RNA encoding luciferase, expression of luciferase was demonstrated at the site of injection measured at 6 hours post dose and was not detected after 9 days. Luciferase was detected to a lesser extent in the liver where expression was present at 6 hours after injection and was not detected by 48 hours after injection. Biodistribution was also assessed after IM administration of a non-metabolizable radiolabeled LNP-mRNA with a comparable lipid composition to the influenza modRNA-bivalent COVID-19 vaccine in Wistar Han rats. Similarly in this study, the percent of administered dose was also greatest at the injection site. Outside of the injection site, total recovery of radioactivity was greatest in the liver and much lower in the spleen, with very little recovery in the adrenal glands and ovaries.

Nonclinical safety of the influenza modRNA-LNP vaccine and the COVID-19 vaccine have been well-characterized separately, including with multiple GLP-compliant repeat-dose toxicity studies and GLP-compliant developmental and reproductive toxicity (DART) studies performed in Wistar Han rats. Although combination toxicity studies with modRNA vaccine and bivalent COVID-19 vaccine have not been conducted, findings in both programs and studies were similar, and included lack of systemic toxicity with expected inflammatory responses to the vaccine. Target organs included the injection site, immune organs (draining and regional lymph nodes, spleen, and bone marrow), and liver in rats. All findings were nonadverse and mostly reversible. Injection site findings included transient erythema and edema, and microscopic mixed cell inflammation with edema. Immune organ effects included macroscopic enlargement of the draining and regional lymph nodes correlating microscopically to increased germinal center cellularity and increased plasma cells; increased spleen weights correlating to germinal center cellularity and increased hematopoiesis; and increased bone marrow hematopoiesis. All immune organ effects were the result of immune stimulation and/or inflammation associated with vaccine administration.

Hepatocellular vacuolation was minimal and reversible without evidence of hepatocyte injury and was likely associated with the LNP uptake. There may be an increase in reactogenicity events compared to the administration of a single vaccine.

### REFERENCES CITED IN EXAMPLE 2

Barry M A, May S, Weaver E A. Imaging luciferase-expressing viruses. Methods Mol Biol 2012; 797:79-87.
Chen C-Y, Tran D M, Cavedon A, et al. Treatment of Hemophilia A Using Factor VIII Messenger RNA Lipid Nanoparticles. Mol Ther Nucleic Acids 2020; 20:534-44.
Elia U, Ramishetti S, Dammes N, et al. Design of SARS-CoV-2 RBD mRNA Vaccine Using Novel Ionizable Lipids. bioRxiv 2020.10.15.341537.
Fukuchi M, Saito R, Maki S, et al. Visualization of activity-regulated BDNF expression in the living mouse brain using non-invasive near-infrared bioluminescence imaging. Mol Brain 2020; 13(1):122.
Hassett K J, Benenato K E, Jacquinet E et al. Optimization of lipid nanoparticles for intramuscular administration of mRNA vaccines. Molecular Therapy Nucleic Acids 2019; 15:1-11.
Jeon Y H, Choi Y, Kang J H, et al. In vivo monitoring of DNA vaccine gene expression using firefly luciferase as a naked DNA. Vaccine 2006; 24(16):3057-62.
Truong B, Allegri G, Liu X-B, et al. Lipid nanoparticle-targeted mRNA therapy as a treatment for the inherited metabolic liver disorder arginase deficiency. Proc Natl Acad Sci USA 2019; 116(42):21150-9.
WHO. Annex 1. Guidelines on the nonclinical evaluation of vaccines. In: Technical Report Series No 927. Geneva, Switzerland: World Health Organization (WHO); 2005. p. 31-63. https_//www.who.int/biologicals/vaccines/nonclinial_evaluation_of_vaccines/en/.

### Example 3: Pharmaceutical Development (Mixtures)

A COVID-19+Influenza mRNA combination vaccine can be prepared by mixing a COVID-19 RNA vaccine (e.g., an LNP-formulated Monovalent (e.g., XBB.1.5-adapted) or Bivalent vaccine (e.g., Wuhan and Omicron (BA.4/BA.5) variant) and Quadrivalent Influenza vaccine (Wisconsin, Phuket, Austria and Darwin) drug product. In the present Example, a COVID-19 Bivalent vaccine and Quadrivalent Influenza vaccine were mixed, but a person of skill in the art will understand that the present Example demonstrates that, in general, different nanoparticle (e.g., LNP) formulated populations can be mixed to form a stable composition.

Both vaccine drug products were preservative-free, sterile dispersions of lipid nanoparticles (LNPs) in aqueous cryoprotectant buffer formulated for intramuscular injection. COVID-19 Monovalent or Bivalent modRNA vaccine can be formulated at 0.1 mg/ml RNA in 10 mM Tris buffer, 10.3% w/v (300 mM) sucrose, pH 7.4. Quadrivalent Influenza modRNA vaccine can be formulated at 0.06 mg/ml RNA in 10 mM Tris buffer, 300 mM sucrose, pH 7.4. Exemplary formulations for each vaccine are provided in the below Tables 36 and 37.

TABLE 36
Composition of the BNT162b2 Bivalent [Original and Omicron (BA.4/BA.5) Variant]
Drug Product, 30 μg total RNA Dose in 0.3 mL Injection Volume, Multi-dose Vial (6 doses)

| | | | | Amount per | | | |
|---|---|---|---|---|---|---|---|
| | | | | 2.25 mL | | | |
| | | | Concen- | Vial^{a} | | | |
| | Reference to | | tration | (MDV = 6 | | Amount per | |
| Name of Ingredient | Standard | Function | (mg/mL) | doses) | | 30 μg Dose | |
| BNT162b2 (Original) drug | In-house | Active | 0.05 | 113 | μg | 15 | μg |
| substance | specification | ingredient | | | | | |
| BNT162b2 Omicron | In-house | Active | 0.05 | 113 | μg | 15 | μg |
| BA.4/BA.5 drug substance | specification | ingredient | | | | | |
| ALC-0315 | In-house | Functional lipid | 1.43 | 3.22 | mg | 0.43 | mg |
| | specification | | | | | | |
| ALC-0159 | In-house | Functional lipid | 0.18 | 0.41 | mg | 0.05 | mg |
| | specification | | | | | | |
| DSPC | In-house | Structural lipid | 0.31 | 0.70 | mg | 0.09 | mg |
| | specification | | | | | | |
| Cholesterol | USP-NF and/or | Structural lipid | 0.62 | 1.40 | mg | 0.19 | mg |
| | Ph. Eur. | | | | | | |
| Sucrose | USP-NF, Ph. | Cryoprotectant | 103 | 231.8 | mg | 31 | mg |
| | Eur. | | | | | | |
| Tromethamine (Tris base)^{b} | USP-NF, Ph. | Buffer | 0.20 | 0.45 | mg | 0.06 | mg |
| | Eur. | component | | | | | |
| Tris (hydroxymethyl) | In-house | Buffer | 1.32 | 2.97 | mg | 0.4 | mg |
| aminomethane | specification | component | | | | | |
| hydrochloride (Tris HCl)^{c} | | | | | | | |

| | | | | | |
|---|---|---|---|---|---|
| Water for Injection | USP-NF, Ph. | Solvent/vehicle | q.s. | q.s. | q.s. |
| | Eur. | | | | |

^{a}Values are rounded to maintain the same level of precision as the label claim, with trailing decimals not shown, where applicable.
^{b}Also known as Trometamol
^{c}Also known as Tromethamine HCl and Trometamol HCl
Abbreviations:
ALC-0315 = ((4-hydroxybutyl)azanediyl)bis(hexane-6,1-diyl)bis(2-hexyldecanoate);
ALC-0159 = 2-[(polyethylene glycol)-2000]-N,N-ditetradecylacetamide;
DSPC = 1,2-distearoyl-sn-glycero-3-phosphocholine;
q.s. = quantum satis (as much as may suffice)

TABLE 37
Composition of 0.06 mg/mL Quadrivalent Drug Product

| | | | | | Nominal |
|---|---|---|---|---|---|
| | | | | | Amount or |
| | | | | Filled | Net |
| | | | Unit | Amount | Quantity |
| | Grade/Quality | | Formula | (Total | (Net |
| Name of Ingredient | Standard | Function | (mg/mL) | mg/vial) | mg/vial) |
| Influenza Drug Substance | In-house | Active | 0.015 | 0.010 | 0.008 |
| (Wisconsin) | specification | ingredient | | | |
| Influenza Drug Substance | In-house | Active | 0.015 | 0.010 | 0.008 |
| (Darwin) | specification | ingredient | | | |
| Influenza Drug Substance | In-house | Active | 0.015 | 0.010 | 0.008 |
| (Austria) | specification | ingredient | | | |
| Influenza Drug Substance | In-house | Active | 0.015 | 0.010 | 0.008 |
| (Phuket) | specification | ingredient | | | |
| ALC-0315^{a} | In-house | Functional lipid | 0.86 | 0.58 | 0.43 |
| | specification | | | | |
| ALC-0159^{b} | In-house | Functional lipid | 0.11 | 0.07 | 0.06 |
| | specification | | | | |
| DSPC^{c} | In-house | Structural lipid | 0.19 | 0.13 | 0.10 |
| | specification | | | | |
| Cholesterol | Ph. Eur., NF | Structural lipid | 0.37 | 0.25 | 0.19 |
| Sucrose | Ph. Eur., NF | Cryoprotectant | 102.69 | 69.83 | 51.35 |
| Tromethamine (Tris base) | Ph. Eur., USP | Buffer | 0.20 | 0.14 | 0.10 |
| | | component | | | |
| Tris (hydroxymethyl) | In-house | Buffer | 1.32 | 0.90 | 0.66 |
| aminomethane | specification | component | | | |
| hydrochloride (Tris HCl) | | | | | |
| Water for Injection | Ph. Eur., USP, JP | Solvent | q.s.^{d} to | q.s.^{d} to | q.s.^{d} to |
| | | | 1.00 mL | 0.68 mL | 0.50 mL |

^{a}ALC-0315 = ((4-hydroxybutyl)azanediyl)bis(hexane-6,1-diyl)bis(2-hexyldecanoate)
^{b}ALC-0159 = 2-[(polyethylene glycol)-2000]-N,N-ditetradecylacetamide
^{c}DSPC = 1,2-Distearoyl-sn-glycero-3-phosphocholine
^{d}q.s. is an abbreviation for quantum satis meaning as much as is sufficient.

### Clinical Done Preparation

For the present study, in some embodiments, the lowest concentration of an individual prepared drug product was 0.023 mg/ml, and the lowest possible combined concentration was 0.069 mg/ml. In some embodiments, the highest concentration of an individual drug product following preparation was 0.055 mg/ml, with a highest possible combined concentration of 0.082 mg/ml. The planned ratios between the drug product doses was 2:1, 1:2, and 1:1. Doses were prepared by mixing the appropriate volumes of the individual vaccines and administering as a single injection of the combined vaccines. Mixing may be done in syringes or glass vials. Details of planned clinical doses are provided in Table 38, below.

TABLE 38
Planned Clinical Doses and Concentrations

| | 60 μg Dose (Group 3) | | | 90 μg Dose (Group 2) | | | 90 μg Dose (Group 1) | | |
|---|---|---|---|---|---|---|---|---|---|
| | (1:1 Influenza:COVID-19) | | | (2:1 Influenza:COVID-19) | | | (1:2 Influenza:COVID-19) | | |
| | Final | Dose | Final | Final | Dose | Final | Final | Dose | Final |
| | Dose | Volume | concentration | Dose | Volume | concentration | Dose | Volume | concentration |
| | (ug) | (mL) | (mg/mL)^{a} | (ug) | (mL) | (mg/mL)^{a} | (ug) | (mL) | (mg/mL)^{a} |
| Quadrivalent | 30 | 0.5 | 0.038 | 60 | 1 | 0.046 | 30 | 0.5 | 0.027 |
| influenza | | | | | | | | | |
| (0.06 mg/mL) | | | | | | | | | |
| COVID Bivalent | 30 | 0.3 | 0.038 | 30 | 0.3 | 0.023 | 60 | 0.6 | 0.055 |
| (0.1 mg/mL) | | | | | | | | | |
| Total | 60 | 0.8 | 0.076 | 90 | 1.3 | 0.069 | 90 | 1.1 | 0.082 |

^{a}The total concentration is calculated by the sum of individual components in the final mixture volume

### Dosage Verification Studies

Dosage verification studies were performed to demonstrate that a Quadrivalent influenza mRNA vaccine drug product and COVID-19 bivalent mRNA vaccine drug product are compatible when mixed for administration, and that all drug product and dosing solutions are compatible with administration components for a period of time adequate to perform dose preparation and administration operations. The dosage verification study design is detailed in the Table 39, below, and is based on a bracketing approach designed to support individual drug product concentrations of 0.02 to 0.067 mg/mL, and combined concentrations of 0.04 to 0.1 mg/mL, as well as the three planned dosing ratios. The stability of the combined vaccine over 24 hours at 2 to 8° C. and 12 hours at 30° C. in dosing syringes was assessed.

TABLE 39
Dosage Verification Study Design

| | Influenza | Covid-19 | | |
|---|---|---|---|---|
| | Quadrivalent | Bivalent | | |
| | Vaccine | Vaccine | Total mRNA | |
| Study Group | Concentration | Concentration | concentration | Ratio |
| Group 1 | 0.033 mg/mL | 0.067 mg/mL | 0.1 mg/mL | 1:2 |
| Group 2 | 0.067 mg/mL | 0.033 mg/mL | 0.1 mg/mL | 2:1 |
| Group 3 | 0.02 mg/mL | 0.02 mg/mL | 0.04 mg/mL | 1:1 |

Samples were tested according to a pre-determined experimental plan and analyzed for appearance, sub-visible particles, pH, RNA content, RNA integrity, RNA encapsulation, LNP size, LNP polydispersity, and ratio (ddPCR) detailed in the below Table 40. Three ratios between the vaccine drug products were tested to bracket the proposed dosing ranges, as described in Table 39.

TABLE 40
Assays and Exemplary Results for the
Administration Simulation Studies

| Assay | Exemplary results |
|---|---|
| Appearance | Visual: White to off-white suspension |
| | Particulate Matter: Essentially Free of |
| | Visible Particles (EFVP) |
| pH | 6.9-7.9 |
| RNA Content (RiboGreen) | T0: Report Result |
| | All other samples: +/−10% of T0 |
| RNA Integrity (CGE by | ≥50% Intact RNA |
| fragment analyzer) | |
| RNA Encapsulation (RiboGreen) | ≥80% |
| LNP Size (DLS) | 40-120 nm |
| LNP PDI (DLS) | ≤0.3 |
| Sequence Ratio (ddPCR) | T0 only; Report Result |

Abbreviations:
CGE = capillary gel electrophoresis;
LNP = lipid nanoparticle;
DLS = Dynamic Light Scattering;
PDI = Polydispersity Index

The results are summarized in Table 41 to Table 43 and demonstrate that the dosing solutions of the combined vaccine drug products are stable for 24 hours, with 12 hours at 30° C. and the remaining time at 2-8° C. in the administration components.

TABLE 41
Dosing Verification Results of Group 1 (1:2 ratio)

| | | | T12 Hours | | T24 Hours | |
|---|---|---|---|---|---|---|
| Quality | Acceptance | T0 | 30° C. | | 2-8° C. | |
| Attribute | Criteria | NA | PP syringe | PC syringe | PP syringe | PC syringe |
| Appearance | Appearance | White to off- | White to off- | White to off- | White to off- | White to off- |
| | Visual: White | white | white | white | white | white |
| | to off-white | suspension | suspension | suspension | suspension | suspension |
| | suspension | | | | | |
| | Appearance | EFVP | EFVP | EFVP | EFVP | EFVP |
| | (Particles): | | | | | |
| | Essentially | | | | | |
| | Free of | | | | | |
| | Visible | | | | | |
| | Particles | | | | | |
| pH | 6.9-7.9 | 7.5 | 7.5 | 7.5 | 7.5 | 7.5 |
| RNA Content | T0: Report | 0.099 | 0.099 (±0%) | 0.098 (−1%) | 0.091 (−8%) | 0.098 (−1%) |
| (mg/mL) | Result | | | | | |
| | All other | | | | | |
| | samples: +/− | | | | | |
| | 10% of T0 | | | | | |
| RNA Integrity | ≥50% Intact | 78 | 77 | 78 | 77 | 78 |
| (%) | RNA | | | | | |
| RNA | ≥80% | 97 | 97 | 96 | 96 | 97 |
| Encapsulation | | | | | | |
| (%) | | | | | | |
| LNP Size (nm) | 40-120 nm | 70 | 70 | 70 | 70 | 70 |
| LNP | ≤0.3 | 0.2 | 0.1 | 0.2 | 0.2 | 0.2 |
| Polydispersity | | | | | | |
| Sequence | Report Result | Total | NT | NT | NT | NT |
| Ratio (%) * | | influenza | | | | |
| | | mRNAs: 40 | | | | |
| | | Total COVID | | | | |
| | | mRNAs: 60 | | | | |

Abbreviations:
EFVP = Essentially Free of Visible Particles;
LNP = lipid nanoparticle;
NT = Not Tested
* Sequence ratio was determined on T0 sample only

TABLE 42
Dosing Verification Results of Group 2 (2:1 ratio)

| | | | T12 Hours | | T24 Hours | |
|---|---|---|---|---|---|---|
| Quality | Acceptance | T0 | 30° C. | | 2-8° C. | |
| Attribute | Criteria | NA | PP syringe | PC syringe | PP syringe | PC syringe |
| Appearance | Appearance | White to off- | White to off- | White to off- | White to off- | White to off- |
| | Visual: White | white | white | white | white | white |
| | to off-white | suspension | suspension | suspension | suspension | suspension |
| | suspension | | | | | |
| | Appearance | EFVP | EFVP | EFVP | EFVP | EFVP |
| | (Particles): | | | | | |
| | Essentially | | | | | |
| | Free of | | | | | |
| | Visible | | | | | |
| | Particles | | | | | |
| pH | 6.9-7.9 | 7.5 | 7.5 | 7.5 | 7.5 | 7.5 |
| RNA Content | T0: Report | 0.102 | 0.098 (−4%) | 0.099 (−3%) | 0.097 (−5%) | 0.102 (±0%) |
| (mg/mL) | Result | | | | | |
| | All other | | | | | |
| | samples: +/− | | | | | |
| | 10% of T0 | | | | | |
| RNA Integrity | ≥50% Intact | 82 | 82 | 82 | 82 | 83 |
| (%) | RNA | | | | | |
| RNA | ≥80% | 96 | 96 | 96 | 95 | 96 |
| Encapsulation | | | | | | |
| (%) | | | | | | |
| LNP Size (nm) | 40-120 nm | 70 | 71 | 70 | 70 | 71 |
| LNP | ≤0.3 | 0.2 | 0.2 | 0.2 | 0.2 | 0.2 |
| Polydispersity | | | | | | |
| Sequence | Report Result | Total | NT | NT | NT | NT |
| Ratio (%) * | | influenza | | | | |
| | | mRNAs: 72 | | | | |
| | | Total COVID | | | | |
| | | mRNAs: 28 | | | | |

Abbreviations:
EFVP = Essentially Free of Visible Particles;
LNP = lipid nanoparticle;
NT = Not Tested
* Sequence ratio was determined on T0 sample only

TABLE 43
Dosing Verification Results of Group 3 (1:1 ratio)

| | | | T12 Hours | | T24 Hours | |
|---|---|---|---|---|---|---|
| Quality | Acceptance | T0 | 30° C. | | 2-8° C. | |
| Attribute | Criteria | NA | PP syringe | PC syringe | PP syringe | PC syringe |
| Appearance | Appearance | White to off- | White to off- | White to off- | White to off- | White to off- |
| | Visual: White | white | white | white | white | white |
| | to off-white | suspension | suspension | suspension | suspension | suspension |
| | suspension | | | | | |
| | Appearance | EFVP | EFVP | EFVP | EFVP | EFVP |
| | (Particles): | | | | | |
| | Essentially | | | | | |
| | Free of | | | | | |
| | Visible | | | | | |
| | Particles | | | | | |
| pH | 6.9-7.9 | 7.5 | 7.5 | 7.6 | 7.5 | 7.5 |
| RNA Content | T0: Report | 0.036 | 0.037 (+3%) | 0.037 (+3%) | 0.038 (+6%) | 0.037 (+3%) |
| (mg/mL) | Result | | | | | |
| | All other | | | | | |
| | samples: +/− | | | | | |
| | 10% of T0 | | | | | |
| RNA Integrity | ≥50% Intact | 80 | 81 | 80 | 81 | 81 |
| (%) | RNA | | | | | |
| RNA | ≥80% | 96 | 96 | 96 | 96 | 96 |
| Encapsulation | | | | | | |
| (%) | | | | | | |
| LNP Size (nm) | 40-120 nm | 70 | 70 | 71 | 71 | 72 |
| LNP | ≤0.3 | 0.2 | 0.2 | 0.2 | 0.2 | 0.2 |
| Polydispersity | | | | | | |
| Sequence | Report Result | Total | NT | NT | NT | NT |
| Ratio (%) * | | influenza | | | | |
| | | mRNAs: 57 | | | | |
| | | Total COVID | | | | |
| | | mRNAs: 43 | | | | |

Abbreviations:
EFVP = Essentially Free of Visible Particles;
LNP = lipid nanoparticle;
NT = Not Tested
* Sequence ratio was determined on T0 sample only

### CONCLUSIONS

Dose preparations should be carried out using aseptic technique in an appropriate environment. Based on the dosage verification studies results described in the present Example, and data from a microbial challenge study conducted on an RNA vaccine having the same formulation, dosing solutions prepared in a controlled and validated aseptic environment should be used within 24 hours from first stopper puncture, with no more than 12 hours at up to 30° C. and the remainder at 2-8° C. If not prepared in a controlled and validated aseptic environment, dosing solutions should be used within 6 hours from first stopper puncture.

### Example 4: Clinical Trial Design for a SARS-CoV-2/Influenza Combination Vaccine

The present Example describes exemplary clinical trial protocols to study combination COVID-19 & influenza RNA vaccines. Specifically, the clinical trial protocols described in the present Example are designed to investigate combinations comprising a bivalent COVID-19 modRNA vaccine and a multivalent influenza modRNA vaccine. The tested combination vaccines are intended for immunization for the prevention of COVID-19 and influenza caused by influenza A and/or B subtype viruses. The exemplary clinical trial protocols focus on participants 18 to 64 years of age to start, and comprise a Phase 1 dose level-finding substudy followed by expanded Phase 2/3 substudies.

Following completion of these substudies, a Phase 2/3 substudy of participants ≥65 years of age is performed. A summary of the design of each substudy is provided below, along with the objectives and endpoints of each phase.

Phase 1: randomized, open-label study of the safety and tolerability and immunogenicity of a combination modRNA vaccine tested at different dose levels in healthy adults 18 to 64 years of age (in the present Example, conducted in the northern hemisphere).

Phase 2/3: evaluation of combination dose levels identified in Phase 1 as compared to current standard of care. Phase 2 to evaluate participants 18 to 64 years of age and 65 years and older. In the present Example, both phases are conducted in the northern hemisphere.

Phase 3: further substudy to be initiated after review of Phase 2 data and to be performed in participants ≥65 years of age (in the present Example, conducted in the southern hemisphere).

### Development Rationale and Plan

Annual vaccine programs against both influenza and COVID-19 are likely to be conducted at a similar time of year in the future. Successful development of a combined vaccine targeting both diseases would likely generate improved vaccination rates for both diseases and allow for more convenient vaccination scheduling, as compared to administration of the vaccines separately.

Separate influenza and COVID-19 vaccines are currently available commercially, but have limitations and there are currently no authorized combination products. Further discussion is provided below regarding the current landscape with regards to Influenza and COVID-19 vaccines.

### Limitations of Current Vaccines

COVID-19 vaccines authorized in the US include two RNA-based products, one adenoviral vector product, and one adjuvanted protein subunit product. Due to observed waning in neutralizing antibody titers over time after a primary series, booster dosing is recommended to restore robust immunity and disease protection. Table 44, below, lists currently approved COVID-19 vaccines.

TABLE 44
Currently Authorized COVID-19 Vaccine
Products in the United States

| Category | Product Name | Manufacturer |
|---|---|---|
| modRNA | BNT162b2; Pfizer-BioNTech | Pfizer/BioNTech^{1, 2} |
| | COVID-19 Vaccine; COMIRNATY ® | |
| | mRNA-1273; Moderna COVID-19 | Moderna³ |
| | Vaccine; Spikevax ™ | |
| adenoviral | JNJ-78436735; Janssen | Janssen⁴* |
| vector | COVID-19 Vaccine* | |
| protein | NVX-CoV2373; Novavax COVID-19 | Novavax⁵* |
| subunit | Vaccine, Adjuvanted* | |

*Note:
Janssen and Novavax COVID-19 vaccines are currently available in the US under emergency use authorization (not licensed) only at this time. Pfizer/BioNTech and Moderna vaccines are licensed as a primary series, and boosters are currently available under emergency use authorization, in the US. Pfizer-BioNTech bivalent vaccine Original & Omicron BA.4/BA.5 booster is currently available in the US under emergency use authorization.
¹Pfizer-BioNTech COVID-19 Vaccine EUA Fact Sheet for Healthcare Providers, Bivalent Booster. Available at: https_//www.fda.gov/media/161327/download/.
²Comirnaty (COVID-19 Vaccine, mRNA) Suspension for Injection for Intramuscular Use. Package Insert. Pfizer (New York, NY, US). Available at: https_//www.fda.gov/media/154834/download.
³Spikevax (COVID-19 Vaccine, mRNA) Suspension for Injection for Intramuscular Use. Package Insert. Moderna (Cambridge, MA, US). Available at: https_//www.fda.gov/media/155675/download.
⁴Janssen COVID-19 Vaccine EUA Fact Sheet for Healthcare Providers. Available at: https_//www.fda.gov/media/146304/download.
⁵Novavax COVID-19 Vaccine, Adjuvanted EUA Fact Sheet for Healthcare Providers. Available at: https_//www.fda.gov/media/159897/download.

Currently approved dosing regimens for BNT162b2 are shown in Table 45, below

TABLE 45
BNT162b2 COVID-19 Vaccine Dose Levels and Regimens by Age Group

| Age Group | Dose Level | Vaccine Regimen |
|---|---|---|
| 6 months to <2 years of age | 3-μg | BNT162b2 three-dose primary series |
| 2 to <5 years of age | 3-μg | BNT162b2 three-dose primary series |
| 5 to <12 years of age | 10-μg | BNT162b2 two-dose primary series and booster |
| ≥12 years of age | 30-μg | BNT162b2 two-dose primary series and booster |
| ≥12 years of age | 30-μg | BNT162b2 bivalent Original & Omicron BA.4/BA.5 booster |

RNA vaccines can require shorter manufacture times than vaccine vectors and traditional vaccine technologies, thus making it easier to generate vaccines against an influenza strain that is prevalent at the time of administration. Moreover, while approximately 70% of eligible individuals in the US have taken a primary series of COVID-19 vaccines, booster dose uptake is significantly lower, and has been reported for <50% of eligible individuals. A combination vaccine can increase rates of booster dosing among eligible individuals.

### Influenza Vaccines

Currently approved influenza vaccines include inactivated, recombinant, and live attenuated influenza vaccines (LAIV) (approved influenza vaccines listed in Table 46, below). Inactivated vaccines are grown in eggs or cell culture before inactivation and may be combined with adjuvant such as MF59 to enhance protection. Vaccines generally contain antigen prepared from three or four influenza viruses (H1N1, H3N2, B Yamagata, and B Victoria) and are termed trivalent or quadrivalent (also known as tetravalent). Potency of inactivated and recombinant vaccines can be expressed in terms of HA content. Standard inactivated vaccines generally contain 15 μg of each HA for adult IM injection, although high-dose inactivated vaccines can contain 60 μg of each HA. LAIV can be delivered by intranasal spray, which is generally used in pediatric populations, and is approved in individuals 2 to 49 years of age in the US and 2 to 17 years of age in Europe.

TABLE 46
Examples of Currently Approved Influenza Vaccine Products
in the United States (Traditional or Accelerated)

| Category | Trade Name | Manufacturer |
|---|---|---|
| Egg-based, inactivated | Fluzone ® | Sanofi Pasteur¹ |
| | Fluzone High-Dose ® | Sanofi Pasteur² |
| | Fluarix ® | GSK³ |
| Egg-based, inactivated, | FluAd ® | Seqirus⁴ |
| with adjuvant (MF59) | | |
| Mammalian cell-based, | Flucelvax ® | Seqirus⁵ |
| inactivated | | |
| Recombinant | FluBlok Quadrivalent ® | Protein Sciences⁶ |
| Live attenuated (LAIV) | FluMist Quadrivalent ® | MedImmune⁷ |

¹Fluzone (Influenza Vaccine) Suspension for Intramuscular Injection. Package Insert. Sanofi Pasteur (Swiftwater, PA, US). Available at: https_//www.fda.gov/media/116102/download.
²Fluzone High-Dose (Influenza Vaccine) Suspension for Intramuscular Injection. Package Insert. Sanofi Pasteur (Swiftwater, PA, US). Available at: https_//www.fda.gov/media/119870/download.
³Fluarix (Influenza Vaccine) Suspension for Intramuscular Injection. Package Insert. GlaxoSmithkline Biologicals (Rixensart, Belgium). Available at: https_//www.fda.gov/media/84804/download.
⁴FluAd (Influenza Vaccine, Adjuvanted) Injectable Emulsion for Intramuscular Use. Package Insert. Seqirus Inc. (Holy Springs, NC, US). Available at: https_//www.fda.gov/media/94583/download.
⁵Flucelvax (Influenza Vaccine) Suspension for Intramuscular Injection. Package Insert. Seqirus Inc. (Holy Springs, NC, US). Available at: https_//www.fda.gov/media/85322/download.
⁶Flublok Quadrivalent (Influenza Vaccine) Sterile Solution for Intramuscular Injection. Package Insert. Protein Sciences Corporation (Meriden, CT, US). Available at: https_//www.fda.gov/media/123144/download.
⁷FluMist Quadrivalent (Influenza Vaccine Live, Intranasal) Intranasal Spray. Package Insert. MedImmune, LLC (Gaithersburg, MD, US). Available at: https_//www.fda.gov/media/120689/download.

Existing influenza vaccines have several limitations, including, among others, relatively long manufacture times, complexity of manufacturing, and limited effectiveness (Bresee et al.). An influenza RNA (e.g., modRNA) vaccine has the potential to address many of these limitations. Specifically, an RNA (e.g., modRNA) vaccine can provide reduced manufacturing time, which allows for a later decision as to the viral strains to include in a seasonal booster campaign, and improve the chances of a vaccine matching the most prevalent circulating strains and offers the potential for no reassortment step (i.e., the improved manufacture time reduces the probability of a vaccine being mismatched with the seasonal circulating strain). SARS-CoV-2 RNA vaccines (e.g., BNT162b2, a modRNA), have also demonstrated high VE against COVID-19, suggesting that influenza RNA vaccines have the potential for higher VE against influenza as compared to currently approved influenza vaccines. In particular, RNA vaccines can offer improved efficacy relative to currently approved vaccines through induction of strong CD4+ and CD8+ T cell responses, similar to those seen with some approved COVID-19 vaccines (e.g., BNT162b2).

Recent CDC data on the 2021-2022 influenza season estimated that as of March 2022, <50% of US adults ≥18 years of age had received an annual influenza vaccination, with uptake in adults ≥65 years of age observed at a higher incidence of nearly 60%.

Advantages of a Combination COVID-19 & Influenza mRNA Vaccine

The combination vaccines studied in the present Example include modRNA active components, which can provide a blunted innate immune sensor activating capacity and thus augmented expression of encoded antigens (e.g., as compared to unmodified RNA and/or saRNA). The modRNA administered in the present Example is encapsulated in LNPs, which enables transfection of the RNA into host cells after IM injection. LNP encapsulation also protects RNA from degradation by RNAses. In the present example, the LNP formulation used for SARS-CoV-2 and influenza components is the same, and composed of four different lipids in a defined ratio.

The combination vaccines of the present Example comprise a bivalent Covid-19 vaccine, comprising two RNAs, each encoding a different SARS-CoV-2 Spike protein antigen (specifically, wildtype (Wuhan) Spike protein and a Spike protein of a SARS-CoV-2 variant that is recommended for priming and/or booster vaccination (in the present Example, an Omicron BA.4/5 variant)). For the multivalent influenza RNA vaccine portion of the combination vaccines, modRNA constructs each encode an HA antigen of a strain recommended for cell-based or recombinant vaccines.

Experience with modRNA-based COVID-19 vaccines supports use of modRNA for rapid development of vaccines, including combination vaccines encoding antigens of emerging SARS-CoV-2 variants and seasonally adapted HAS for prevention of influenza. Potential advantages of RNA (e.g., modRNA) vaccines compared to other types of vaccines include, among others, accelerated manufacturing (thereby allowing a later decision as to the viral strains to include in a seasonal booster campaign, which can increase the chances that a booster vaccine will match the relevant influenza strain). RNA vaccines developed against SARS-CoV-2 (e.g., BNT162b2) have also provided evidence of high VE against COVID-19, suggesting that RNA vaccines have the potential for higher VE against influenza relative to currently licensed influenza vaccines.

Further advantages of RNA-based vaccines as compared to other vaccine approaches are listed below:
RNA-based vaccines do not carry a risk of infection.
RNA-based vaccines can mimic antigen expression during natural infection by directing expression of a pathogen antigen with high precision and flexibility of antigen design.
RNA occurs naturally in the body, is metabolized and eliminated by the body's natural mechanisms, does not integrate into the genome, and is transiently expressed.

RNA-based vaccines can be manufactured using a cell-free in vitro transcription process, which can allow for easy and rapid production and the potential for producing high numbers of vaccine doses within a shorter time-period than can be achieved with conventional vaccine approaches. This capability is important for enabling efficient delivery of a seasonally updated vaccine.

Clinical investigation of a combination modRNA vaccine targeting influenza and COVID-19 is justified given (1) continued public health threat posed by the ongoing global SARS COV 2 outbreak and emergence of variants of concern, (2) need for boosters to overcome waning immunity, and (3) potential advantages to individuals including convenience of receiving a combined booster vaccine against both influenza and SARS CoV-2 that may be best matched to identified circulating strains. In some embodiments, combination vaccines can offer protection against emerging SARS-CoV-2 variants and seasonally adapted HAs for prevention of influenza, which could decrease the number of vaccinations needed, simplify programmatic challenges, and ultimately increase adherence to a recommended schedule for influenza and COVID-19 vaccination (e.g., as compared to vaccines administered separately). Each of these advantages (separately or together) could result in an increased proportion of individuals being vaccinated, leading to an improved effect on serious outcomes of influenza and COVID-19.

### General Approach to Combination Vaccine Development

The present study comprises a first Phase I substudy designed to evaluate and select an optimal dose level of a combination vaccine comprising a quadravalent influenza vaccine and a bivalent COVID-19 modRNA vaccine, for later-phase development. Phase 2/3 and Phase 3 substudies can be initiated once data are available from the Phase 1 substudy, and are designed to further evaluate selected combination dose levels for safety and noninferiority of immune response as compared to current standard of care (co-administered quadrivalent influenza vaccine and bivalent vaccine).

### Planned Phase 1 Study (Substudy A)

Substudy A is a Phase 1 randomized, open-label study to evaluate the safety and immunogenicity of a combination COVID-19 & influenza mRNA vaccine administered IM at different dose level combinations in healthy adults 18 to 64 years of age (dosages listed in Table 47). In this study, 30 participants each are randomized 1:1:1:1:1:1 to one of six groups to receive a single study vaccination as follows:
Combination vaccine: qIRV encoding antigens from four selected strains from 2022/2023 influenza season (22/23) combined with a bivalent COVID-19 vaccine (BNT162b2) encoding an antigen from the original (Wuhan) strain and an antigen from the Omicron (OMI) BA.4/BA.5 variant of SARS-CoV-2, at one of three dose level combinations (Groups #1, #2, and #3; dose levels listed in Table 47).
Influenza modRNA vaccine: 30-μg qIRV (22/23) (Group #4)
Influenza modRNA vaccine: 60-μg qIRV (22/23) (Group #5)
Standard of care: co-administered licensed QIV+bivalent BNT162b2 Wuhan+OMI BA.4/BA.5 at a 30-μg dose, vaccines administered concurrently in opposite arms (Group #6)

Phase 1 enrollment includes at most 10 participants to be vaccinated on the first day with further vaccinations no sooner than 24 hours thereafter. Dose level combinations #1 and/or #3 with a total dose of 90-μg modRNA may be omitted from this study depending on review of emerging data. Clinical data from prior studies of BNT162b2 at dose levels of 30-μg or 60-μg are planned to be used as an historical control in the combination vaccine study analysis.

TABLE 47
Phase 1 Study Interventions and Dose Level Combinations

| Group | N per | qIRV | BNT162b2 Bivalent | Total Dose |
|---|---|---|---|---|
| Number | Group | (22/23) Dose | (Original/OMI BA.4/BA.5) Dose | modRNA |
| 1 | 30 | 30-μg | 60-μg | 90-μg |
| | | 2 type A strains + 2 type B strains, | 30-μg of BNT162b2 Original + | |
| | | dose of 7.5-μg per strain | 30-μg of BNT162b2 OMI BA.4/BA.5 | |
| 2 | 30 | 30-μg | 30-μg | 60-μg |
| | | 2 type A strains + 2 type B strains, | 15-μg of BNT162b2 Original + | |
| | | dose of 7.5-μg per strain | 15-μg of BNT162b2 OMI BA.4/BA.5 | |
| 3 | 30 | 60-μg | 30-μg | 90-μg |
| | | 2 type A strains + 2 type B strains, | 15-μg of BNT162b2 Original + | |
| | | dose of 15-μg per strain | 15-μg of BNT162b2 OMI BA.4/BA.5 | |
| 4 | 30 | 30-μg | NA | 30-μg |
| | | 2 type A strains + 2 type B strains, | | |
| | | dose of 7.5-μg per strain | | |
| 5 | 30 | 60-μg | NA | 60-μg |
| | | 2 type A strains + 2 type B strains, | | |
| | | dose of 15-μg per strain | | |
| 6 | 30 | NA | 30-μg | 30-μg |
| (standard | | | 15-μg of BNT162b2 Original + | |
| of care) | | | 15-μg of OMI BA.4/BA.5 administered | |
| | | | concurrently in the opposite arm to | |
| | | | licensed QIV | |

NA = not applicable; OMI BA.4/BA.5 = SARS-CoV-2 Omicron variant sublineage; qIRV = quadrivalent influenza modRNA vaccine; QIV = quadrivalent influenza vaccine; 22/23 = 2022/2023 influenza season recommended strains.

TABLE 48
Objectives, Endpoints, and Estimands of Substudy A Phase 1:

| Objectives | Estimands | Endpoints |
|---|---|---|
| Primary Safety: | Primary Safety: | Primary Safety: |
| To describe the safety and | In participants receiving at least 1 dose of | Local reactions (pain at the |
| tolerability of qIRV | study intervention, the percentage of | injection site, redness, and |
| (22/23)/bivalent | participants reporting: | swelling) |
| BNT162b2 (original/Omi | Local reactions for up to 7 days | Systemic events (fever, fatigue, |
| BA.4/BA.5) at various | following vaccination | headache, chills, vomiting, |
| dose-level combinations | Systemic events for up to 7 days | diarrhea, new or worsened |
| | following vaccination | muscle pain, and new or |
| | AEs from the first vaccination through | worsened joint pain) |
| | 4 weeks after vaccination | AEs |
| | SAEs from the first vaccination through | SAEs |
| | 6 months after vaccination | |
| | The percentage of participants with: | Troponin I laboratory parameters |
| | Abnormal troponin I laboratory values | |
| | 2 days and 1 week after vaccination | |
| | The percentage of participants with: | ECG abnormalities consistent with |
| | New ECG abnormalities 2 days and 1 | probable or possible myocarditis or |
| | week after vaccination | pericarditis |
| Secondary: | Secondary: | Secondary: |
| To describe the immune | In participants complying with the key | HAI titers for the 2022/2023 seasonal |
| responses elicited by qIRV | protocol criteria (evaluable participants): | strains (2 × A, 2 × B) recommended by |
| (22/23)/bivalent | GMTs before vaccination and at 1, 4, | WHO for recombinant or cell-based |
| BNT162b2 (original/Omi | and 8 weeks after vaccination | influenza vaccines |
| BA.4/BA.5) at various | GMFR from before vaccination to 1, 4, | |
| dose-level combinations | and 8 weeks after vaccination | |
| | The proportion of participants achieving | |
| | HAI seroconversion^{a} for each strain at | |
| | 1, 4, and 8 weeks after vaccination | |
| | The percentage of participants with HAI | |
| | titers ≥1:40 for each strain before | |
| | vaccination and at 1, 4, and 8 weeks | |
| | after vaccination | |
| | The percentage of participants | |
| | achieving HAI seroconversion for all | |
| | strains at 1, 4, and 8 weeks after | |
| | vaccination | |
| | The percentage of participants with HAI | |
| | titers ≥1:40 for all strains at 1, 4, and | |
| | 8 weeks after vaccination | |
| | In participants having received qIRV | SARS-CoV-2 Omicron |
| | (22/23)/bivalent BNT162b2 (original/Omi | (BA.4/BA.5)-neutralizing titers |
| | BA.4/BA.5) complying with the key | SARS-CoV-2 reference-strain- |
| | protocol criteria (evaluable participants): | neutralizing titers |
| | GMTs before vaccination and at 1, 4, | |
| | and 8 weeks after vaccination for each | |
| | strain-specific neutralizing titer. | |
| | GMFR from before vaccination to 1, 4, | |
| | and 8 weeks after vaccination for each | |
| | strain-specific neutralizing titer. | |
| | Percentages of participants with | |
| | seroresponse^{b} at 1, 4, and 8 weeks | |
| | after vaccination for each strain-specific | |
| | neutralizing titer | |
| Exploratory | Exploratory | Exploratory |
| To describe the immune | | SARS-CoV-2-neutralizing titers |
| response to emerging | | for VOCs not already specified |
| VOCs. | | |

^{a}Seroconversion is defined as an HAI titer <1:10 prior to vaccination and ≥1:40 at the time point of interest, or an HAI titer of >1:10 prior to vaccination with a 4-fold rise at the time point of interest.
^{b}Seroresponse is defined as achieving a ≥4-fold rise from baseline (before the study vaccination). If the baseline measurement is below the LLOQ, the postvaccination measure of ≥4 × LLOQ is considered seroresponse.

### Justification for Dose

qIRV (22/23)/bivalent BNT162b2 (original/Omi BA.4/BA.5) at dose-level combinations not exceeding 90 μg is evaluated in the study described in the present Example.

The following conditions may allow a participant to be randomized once the conditions have resolved and the participant is otherwise considered eligible. Participants meeting these criteria at Visit 1 are considered screen failures if enrollment has closed once the condition(s) has/have resolved.
1. A positive SARS-CoV-2 test result (NAAT or rapid antigen test) within the previous 28 days.
2. Current febrile illness (oral temperature ≥38.0° C. [≥100.4° F.]) or other acute illness within 48 hours before study intervention administration. This includes symptoms that could represent a potential COVID-19 illness.
   Note: The participant should be directed to seek additional testing through his/her primary healthcare provider at a licensed clinical laboratory when exhibiting potential COVID-19 symptoms or otherwise receiving a positive result and counseled on whether to take any precautionary measures pending confirmatory testing, as per local guidance.
3. Receipt of any nonstudy vaccine within 28 days before study intervention administration at Visit 1.
4. Anticipated receipt of any nonstudy vaccine within 28 days after study intervention administration at Visit 1.
5. Receipt of short-term (<14 days) systemic corticosteroids. Study intervention administration should be delayed until systemic corticosteroid use has been discontinued for at least 28 days. Inhaled/nebulized, intra-articular, intrabursal, or topical (skin or eyes) corticosteroids are permitted.

### Antipyretic Medication

The use of antipyretic medication to treat symptoms associated with study intervention administration is recorded in the reactogenicity e-diary daily during the reporting period (Day 1 through Day 7).

For Substudy A, the following concomitant medications and vaccinations are recorded in the CRF:
Prior receipt of any COVID-19 vaccine.
Prior receipt of any pneumococcal vaccine.
Licensed influenza vaccine, if received during the prior calendar year.
Any vaccinations received from 28 days prior to study enrollment until the last visit (Visit 6).
Prohibited medications (listed below), if taken, are recorded and include start and stop dates, name of the medication, dose, unit, route, and frequency.

### Prohibited During the Study

Receipt of the following vaccines and medications during the time periods listed below may exclude a participant from the per-protocol analysis from that point onward; however, it is anticipated that the participant would not be withdrawn from the study. Medications should not be withheld if required for a participant's medical care.
Unless considered medically necessary, no vaccines other than study intervention should be administered within 28 days before and 28 days after study vaccination at Visit 1.
Receipt of any other (nonstudy) coronavirus vaccine from enrollment through Visit 5 (8-week follow-up visit) is prohibited.
Receipt of any other (nonstudy) seasonal influenza vaccine from enrollment through Visit 5 (8-week follow-up visit) is prohibited.
Receipt of chronic systemic treatment with known immunosuppressant medications, or radiotherapy, within 60 days before enrollment through conclusion of the study is prohibited.
Receipt of systemic corticosteroids (≥20 mg/day of prednisone or equivalent) for ≥14 days is prohibited from 28 days prior to enrollment through 28 days after administration of the study intervention.
Receipt of blood/plasma products, immunoglobulins, or monoclonal antibodies, from 60 days before study intervention administration through conclusion of the study.
Prophylactic antipyretics and other pain medication to prevent symptoms associated with study intervention administration are not permitted. However, if a participant is taking a medication for another condition, even if it may have antipyretic or pain-relieving properties, it should not be withheld prior to study vaccination.

### Permitted During the Study

Medication other than that described as prohibited (listed above) required for treatment of preexisting conditions or acute illness is permitted.
Inhaled, topical, or localized injections of corticosteroids (e.g., intra-articular or intrabursal administration) are permitted.

### Immunogenicity Assessments

Samples are collected at time points as specified above from all participants, and the following assays run:
HAI titers against seasonal strains (2×A, 2×B) recommended by WHO for recombinant or cell-based influenza vaccines
SARS-CoV-2 neutralization assay (reference strain)
SARS-CoV-2 neutralization assays (Omicron BA.4, Omicron BA.5; other VOCs of interest, including other Omicron sublineages, may also be evaluated)

### N-Binding Antibody Test

The N-binding antibody test is performed by the central laboratory on each blood sample to establish prior exposure to SARS-CoV-2 up to each time point. These data are used for study analyses.

### Substudy A (Phase 1) Study Population

### Inclusion Criteria

Participants are eligible to be included in Substudy A only if all of the following criteria apply:

### Age and Sex:

1. Male or female participants 18 through 64 years of age at Visit 1 (Day 1).
2. Participants who are willing and able to comply with all scheduled visits, investigational plan, laboratory tests, lifestyle considerations, and other study procedures.
3. Healthy participants who are determined by medical history, physical examination (if required), and clinical judgment of the investigator to be eligible for inclusion in the study.
   Note: Healthy participants with preexisting stable disease, defined as disease not requiring significant change in therapy or hospitalization for worsening disease during the 6 weeks before enrollment, can be included.
4. Capable of giving signed informed consent as described in the protocol, which includes compliance with the requirements and restrictions listed in the ICD and in this protocol.
5. Participants who have received 3 prior doses of 30 μg BNT162b2, with the last dose being 150 to 365 days before Visit 1 (Day 1).
   Note: Documented confirmation of prior doses of BNT162b2 received must be obtained prior to randomization.

### Exclusion Criteria

Participants are excluded from Substudy A if any of the following criteria apply:

### Medical Conditions:

1. History of severe adverse reaction associated with any vaccine and/or severe allergic reaction (e.g., anaphylaxis) to any component of the study intervention(s).
2. Immunocompromised individuals with known or suspected immunodeficiency, as determined by history and/or laboratory/physical examination.
3. Bleeding diathesis or condition associated with prolonged bleeding that would, in the opinion of the investigator, contraindicate intramuscular injection.
4. Women who are pregnant or breastfeeding.
5. Allergy to egg proteins (egg or egg products) or chicken proteins.
6. Other medical or psychiatric condition including recent (within the past year) or active suicidal ideation/behavior or laboratory abnormality that may increase the risk of study participation or, in the investigator's judgment, make the participant inappropriate for the study.

### Prior/Concomitant Therapy:

7. Receipt of chronic systemic treatment with known immunosuppressant medications (including cytotoxic agents or systemic corticosteroids), or radiotherapy, within 60 days before enrollment through conclusion of the study.
   Note: Systemic corticosteroids are defined as those administered for ≥14 days at a dose of ≥20 mg/day of prednisone or equivalent (e.g., for cancer or an autoimmune disease) or planned receipt throughout the study. Inhaled/nebulized, intra-articular, intrabursal, or topical (skin or eyes) corticosteroids are permitted.
8. Receipt of blood/plasma products, immunoglobulin, or monoclonal antibodies, from 60 days before study intervention administration, or planned receipt throughout the study.
9. Vaccination with any investigational or licensed influenza vaccine within 6 months (175 days) before study intervention administration.

### Prior/Concurrent Clinical Study Experience:

10. Participation in other studies involving a study intervention within 28 days before randomization. Anticipated participation in other studies within 28 days after receipt of study intervention in this study.

### Other Exclusion Criteria:

11. Investigator site staff directly involved in the conduct of the study and their family members, site staff otherwise supervised by the investigator, and sponsor and sponsor delegate employees directly involved in the conduct of the study and their family members.
12. Participation in strenuous or endurance exercise through Visit 3.
13. Prior history of heart disease.
14. Any abnormal screening troponin I laboratory value.
15. Screening 12-lead ECG that, as judged by the investigator, is consistent with probable or possible myocarditis or pericarditis, or demonstrates clinically relevant abnormalities that may affect participant safety or interpretation of study results. Participants with a screening 12-lead ECG that shows an average QTcF interval >450 msec, complete left bundle branch block, signs of an acute or indeterminate-age myocardial infarction, ST-T interval changes suggestive of myocardial ischemia, second- or third-degree AV block, or serious bradyarrhythmias or tachyarrhythmias should be excluded from study participation.

### Study Arms and Duration:

### Study Interventions Administered

### Substudy A (Phase 1)

| | | | | |
|---|---|---|---|---|
| Intervention | Bivalent BNT162b2 | qIRV (22/23) | qIRV (22/23)/bivalent | QIV |
| Name | (original/Omi BA.4/BA.5) | | BNT162b2 | |
| | (original BNT162b2 and | | (original/Omi BA.4/BA.5) | |
| | BNT162b2 Omicron | | | |
| | [B.1.1.529 sublineage | | | |
| | BA.4/BA.5]) | | | |
| | Preformulated as a | | | |
| | single vial (no dilution | | | |
| | required) | | | |
| Arm Name | qIRV (22/23)/bivalent | qIRV (22/23)/bivalent | qIRV (22/23)/bivalent | |
| (group of | BNT162b2 | BNT162b2 | BNT162b2 | |
| participants | (original^{a}/Omi BA.4/BA.5) | (original/Omi BA.4/BA.5) | (original/Omi BA.4/BA.5) | |
| receiving a | or Licensed QIV + | or | | |
| specific study | bivalent BNT162b2 | 30 μg qIRV (22/23) | | |
| intervention | (original/Omi BA.4/BA.5) | Or | | |
| or no study | | 60 μg qIRV (22/23) | | |
| intervention) | | | | |
| Type | Vaccine | Vaccine | Vaccine | Vaccine |
| Dose | modRNA | modRNA | modRNA | |
| Formulation | | | | |
| Unit Dose | 30 μg/0.3 mL | 30 μg/0.3 mL | Dose-level combinations 1 | As detailed in the IPM |
| Strength(s) | 60 μg/0.6 mL | 60 μg/0.6 mL | and 3 | |
| | | | 90 μg/0.9 mL | |
| | | | Dose-level combination 2 | |
| | | | 60 μg/0.6 mL | |
| Dosage | 30 μg or 60 μg | 30 μg | Dose-level combination 1 | |
| Level(s) | (15 μg original | (7.5 μg per strain) | 90 μg | |
| | BNT162b2 and | 60 μg | qIRV (22/23) 30 μg | |
| | 15 μg BNT162b2 | (15 μg per strain) | (7.5 μg per strain) | |
| | Omicron | | BNT162b2 60 μg (30 μg | |
| | [B.1.1.529 sublineage | | original BNT162b2 and | |
| | BA.4/BA.5]) | | 30 μg BNT162b2 Omicron | |
| | (30 μg original | | [B.1.1.529 sublineage | |
| | BNT162b2 and | | BA.4/BA.5]) | |
| | 30 μg BNT162b2 | | Dose-level combination 2 | |
| | Omicron | | 60 μg | |
| | [B.1.1.529 sublineage | | qIRV (22/23) 30 μg | |
| | BA.4/BA.5]) | | (7.5 μg per strain) | |
| | | | BNT162b2 30 μg (15 μg | |
| | | | original BNT162b2 and | |
| | | | 15 μg BNT162b2 Omicron | |
| | | | [B.1.1.529 sublineage | |
| | | | BA.4/BA.5]) | |
| | | | Dose-level combination 3 | |
| | | | 90 μg | |
| | | | qIRV (22/23) 60 μg | |
| | | | (15 μg per strain) | |
| | | | BNT162b2 30 μg (15 μg | |
| | | | original BNT162b2 and | |
| | | | 15 μg BNT162b2 Omicron | |
| | | | [B.1.1.529 sublineage | |
| | | | BA.4/BA.5]) | |
| Route of | Intramuscular | Intramuscular | Intramuscular | Intramuscular |
| Administration | injection | injection | injection | injection |
| Use | Experimental | Experimental | Experimental | Comparator |
| Sourcing | Provided centrally | Provided centrally | Study intervention is | Provided centrally |
| Packaging | Study intervention is | Study intervention is | generated by mixing the | Study intervention is |
| and Labeling | provided in a glass | provided in a glass | following at the site at the | provided as either |
| | vial as open-label | vial as open-label | dose-level combinations | a PFS or a glass |
| | supply. Each vial is | supply. Each vial is | detailed above: | vial as open-label |
| | labeled per country | labeled per country | qIRV (22/23) | supply. Each vial is |
| | requirement. | requirement. | Bivalent BNT162b2 | labeled per country |
| | | | (original/Omi BA.4/BA.5) | requirement. |

^{a}Original refers to the ancestral strain (Wuhan-Hu-1; USA-WA1/2020), also referred to herein as the reference strain.

Study Arms

| Group | | | | | | |
|---|---|---|---|---|---|---|
| Number | 1 | 2 | 3 | 4 | 5 | 6 |
| Arm Title | qIRV | qIRV | qIRV | 30 μg qIRV | 60 μg qIRV | Licensed |
| | (22/23)/bivalent | (22/23)/bivalent | (22/23)/bivalent | (22/23) | (22/23) | QIV+ |
| | BNT162b2 | BNT162b2 | BNT162b2 | | | bivalent |
| | (original/Omi | (original/Omi | (original/Omi | | | BNT162b2 |
| | BA.4/BA.5) | BA.4/BA.5) | BA.4/BA.5) | | | (original/Omi |
| | Dose-level | Dose-level | Dose-level | | | BA.4/BA.5) |
| | combination 1 | combination 2 | combination 3 | | | |
| Arm Type | Experimental | Experimental | Experimental | Experimental | Experimental | Experimental |
| Arm | Participants | Participants | Participants | Participants | Participants | Participants |
| Description | receive qIRV | receive IRV | receive qIRV | receive 30 | receive 60 | receive 30 |
| | (22/23)/bivalent | (22/23)/bivalent | (22/23)/bivalent | μg of qIRV | μg of qIRV | μg of |
| | BNT162b2 | BNT162b2 | BNT162b2 | (22/23) | (22/23) | bivalent |
| | (original/Omi | (original/Omi | (original/Omi | | | BNT162b2 |
| | BA.4/BA.5) | BA.4/BA.5) | BA.4/BA.5) | | | (original/Omi |
| | at dose-level | at dose-level | at dose-level | | | BA.4/BA.5) |
| | combination 1 | combination 2 | combination 3 | | | administered |
| | | | | | | concurrently |
| | | | | | | in the |
| | | | | | | opposite arm |
| | | | | | | to QIV |
| Associated | qIRV | qIRV | qIRV | qIRV | qIRV | Bivalent |
| Intervention | (22/23)/bivalent | (22/23)/bivalent | (22/23)/bivalent | (22/23) | (22/23) | BNT162b2 |
| Labels | BNT162b2 | BNT162b2 | BNT162b2 | | | (original/Omi |
| | (original/Omi | (original/Omi | (original/Omi | | | BA.4/BA.5) |
| | BA.4/BA.5) | BA.4/BA.5) | BA.4/BA.5) | | | and |
| | | | | | | QIV |

### Statistical Methods:

### Substudy A (Phase 1)

Since this substudy is descriptive in nature, the planned sample size for the substudy is not based on any statistical hypothesis testing.

Safety and immunogenicity data from studies previously conducted in participants of a similar age range having received 1 dose of BNT162b2 at a dose level of 30 μg and 60 μg may be used as a control during the study analysis.

The primary safety objective for the study is evaluated by descriptive summary statistics for local reactions, systemic events, and AEs/SAEs for each vaccine group. Abnormal troponin I laboratory parameters and ECG abnormalities consistent with probable or possible myocarditis or pericarditis is also descriptively summarized for the primary safety objective.

The secondary (immunogenicity) objective is evaluated descriptively by GMT and GMFR of both HAI and SARS-CoV-2 neutralizing titers, as well as:
the percentage of participants achieving seroconversion measured by HAI, and proportion of participants with HAI titers ≥1:40, for each strain at the various time points.
the percentage of participants with seroresponse of SARS-CoV-2 neutralizing titers at the various time points.

### Phase 2/3 Study

The Phase 2/3 substudy is initiated after review of Phase 1 data and is designed to evaluate safety and noninferiority of immune responses elicited by the combination vaccine at the selected dose levels versus standard of care. Phase 2 evaluation includes participants ≥18 years of age, including 30 or 120 participants per group. Phase 3 evaluations include participants 18 to 64 years of age randomized at a 2:1 ratio to include 3000 participants in the combination vaccine group and 1500 participants in the standard of care comparator group. This Phase 2/3 study is conducted in the northern hemisphere in winter 2022/2023. Dosages to be tested in this Phase 2/3 study, are listed in the below Tables 49A and 49B.

TABLE 49A
Exemplary dosing regimens for investigation in a Phase 2/3 Study (for subjects 18-64 years old)

| | | | Injection 2 | |
|---|---|---|---|---|
| Group | N | Injection 1 | (Alternate Arm) | Formulation |
| 1 | 30 | Flu¹/COVID²: 30 | N/A | Bedside mix (influenza and Covid |
| | | μg/30 μg | | components mixed immediately |
| | | Flu: 7.5 μg each | | prior to administration) |
| | | 2 × As, 2 × Bs | | |
| 2 | 120 | Flu¹/COVID²: 60 | N/A | |
| | | μg/30 μg | | |
| | | Flu: 15 μg each | | |
| | | 2 × As, 2 × Bs | | |
| 3 | 120 | Flu¹/COVID²: 60 | N/A | Co-formulated (single LNP co- |
| | | μg/30 μg | | encapsulating all RNAs) |
| | | Flu: 15 μg each | | |
| | | 2 × As, 2 × Bs | | |
| 4 | 30 | Flu¹: 30 μg | N/A | |
| | | Flu: 7.5 μg each | | |
| | | 2 × As, 2 × Bs | | |
| 5 | 30 | QIV (Flucelvax) | COVID² (30 μg) | N/A |
| 6 | 30 | QIV(Flucelvax) | Flu AA/COVID: 15 μg/ | |
| | | | 30 μg | |
| | | | Flu: 7.5 μg each 2 × As | |
| 7 | 30 | Flu¹/COVID²: 30/30 | N/A | Bedside mix |
| | | Flu: 7.5 μg each | | |
| | | 2 × As, 2 × Bs | | |
| 8 | 30 | Flu¹/COVID²: 45/30 | N/A | |
| | | Flu: 11.25 μg each | | |
| | | 2 × As, 2 × Bs | | |
| 9 | 30 | Flu/COVID²: 60 μg/ | N/A | |
| | | 30 μg | | |
| | | Flu: 5 μg 2 × As, | | |
| | | 25 μg 2 × Bs | | |
| 10 | 30 | Flu/COVID²: 30 μg/ | N/A | |
| | | 30 μg | | |
| | | Flu: 2.5 μg 2 × As, | | |
| | | 12.5 μg 2 × Bs | | |
| 11 | 30 | Flu/COVID²: 30 μg/ | N/A | |
| | | 60 μg | | |
| | | Flu: 2.5 μg 2 × As, | | |
| | | 12.5 μg 2 × Bs | | |
| 12 | 30 | Flu/COVID²: 45 μg/30 | N/A | |
| | | μg. | | |
| | | Flu (AAB): 7.5 μg | | |
| | | 2 × As, 30 μg 1 × B | | |
| 13 | 30 | Flu/COVID²: 30 μg/ | N/A | |
| | | 30 μg | | |
| | | Flu: AAAB (7.5 μg | | |
| | | 3 × As, 1 × B) | | |

¹Quadrivalent influenza vaccine administered, comprising 4 RNAs, two encoding an HA polypeptide from an influenza type A strain and two encoding an HA polypeptide from an influenza type B strain.
²A bivalent SARS-CoV-2 vaccine is administered, comprising a first RNA encoding an S protein from a first SARS-CoV-2 strain or variant (Wuhan in the present Example), and a second RNA encoding an S protein from a second SARS-CoV-2 strain or variant (Omicron BA.4/5 in the present Example). In some embodiments, the two RNAs are present at a 1:1 ratio.

TABLE 49B
Exemplary dosing regimens for investigation in a
Phase 2/3 Study (for subjects 65 years and older)

| | | | Injection 2 | |
|---|---|---|---|---|
| Group | N | Injection 1 | (Alternate Arm) | Formulation |
| 1 | 30 | Flu¹/COVID²: 30 | N/A | |
| | | μg/30 μg | | |
| 2 | 30 | Flu¹/COVID²: 60 | N/A | Bedside mix (influenza and Covid |
| | | μg/30 μg | | components mixed immediately |
| | | | | prior to administration) |
| 3 | 30 | Flu¹/COVID²: 60 | N/A | Co-formulated (single LNP co- |
| | | μg/30 μg | | encapsulating all RNAs) |
| 4 | 30 | Flu¹: 60 μg | N/A | |
| 5 | 30 | QIV (Flucelvax) | COVID² (30 μg) | Bedside mix |
| 6 | 30 | QIV(Flucelvax) | Flu AA/COVID: | |
| | | | 15 μg/30 μg | |
| 7 | 30 | Flu/COVID²: 45 μg/ | N/A | |
| | | 30 μg | | |
| | | Flu: 11.25 μg each | | |
| | | 2 × As, 2 × Bs | | |
| 8 | 30 | Flu/COVID²: 60 μg/ | N/A | |
| | | 30 μg | | |
| | | Flu: 5 μg 2 × As, | | |
| | | 25 μg 2 × Bs | | |
| 9 | 30 | Flu/COVID²: 30 μg/ | N/A | |
| | | 30 μg | | |
| | | Flu: 2.5 μg 2 × As, | | |
| | | 12.5 μg 2 × Bs | | |
| 10 | 30 | Flu/COVID²: 30 μg/ | N/A | |
| | | 60 μg | | |
| | | Flu: 2.5 μg 2 × As, | | |
| | | 12.5 μg 2 × Bs | | |
| 11 | 30 | Flu/COVID²: 45 μg/ | N/A | |
| | | 30 μg. | | |
| | | Flu (AAB): 7.5 μg | | |
| | | 2 × As, 30 μg 1 × B | | |
| 12 | 30 | Flu/COVID²: 30 μg/ | N/A | |
| | | 30 μg | | |
| | | Flu: AAAB (7.5 μg | | |
| | | 3 × As, 1 × B) | | |

¹Quadrivalent influenza vaccine administered, comprising 4 RNAs, two encoding an HA polypeptide from an influenza type A strain and two encoding an HA polypeptide from an influenza type B strain.
²A bivalent SARS-CoV-2 vaccine is administered, comprising a first RNA encoding an S protein from a first SARS-CoV-2 strain or variant, and a second RNA encoding an S protein from a second SARS-CoV-2 strain or variant. In some embodiments, the two RNAs can be present at a 1:1 ratio.

In some embodiments, each RNA in a composition can be encapsulated in a separate LNP. In some embodiments, one or more RNAs encoding antigens from a first disease can be co-encapsulated in a first LNP, and one or more RNAs encoding antigens from a second disease can be co-encapsulated in a second LNP (e.g., for a composition comprising one or more RNAs encoding a SARS-CoV-2 antigen and one or more antigens encoding an influenza antigen, each RNA encoding a SARS-CoV-2 antigen can be co-formulated in a first LNP, and each RNA encoding an influenza antigen can be co-formulated in a second LNP, and the first and second LNPs can be mixed prior to administering). In some embodiments, each RNA encoding an antigen from a first disease can be co-formulated in the same LNP, and each RNA encoding an antigen from a second disease can be co-formulated in separate LNPs (e.g., for a composition comprising two or more RNAs encoding influenza antigens, and two or more RNAs encoding SARS-CoV-2 antigens, the two or more RNAs encoding influenza antigens can each be formulated in separate LNPs, and the two or more RNAs encoding SARS-CoV-2 antigens can be co-formulated in the same LNP, and all the LNPs can be mixed prior to administering).

### Phase 3 Study

A further Phase 3 substudy is designed after review of Phase 2 data to evaluate safety and noninferiority of immune responses elicited by the combination vaccine versus standard of care in participants ≥65 years of age, randomized at a 2:1 ratio to include 3000 participants in the combination vaccine group and 1500 participants in the standard of care comparator group. This Phase 3 study is conducted in the southern hemisphere.

### Overview of Biopharmaceutics

The combination COVID-19 & influenza mRNA vaccine tested in the present example includes two vaccines: qIRV (22/23) and bivalent BNT162b2 (Wuhan+OMI BA.4/BA.5). The drug product (DP) formulation for each vaccine in the developmental combination product is summarized below. In the Phase 1 and 2 studies, DP can be mixed at the study site or a co-formulation can be used. In Phase 3, a co-formulation DP can be used. Formulations of the individual vaccines characterized in the present Example are provided below:
Bivalent COVID-19 vaccine (BNT162b2 Wuhan+OMI BA.4/BA.5) is a preservative-free, sterile dispersion of LNPs in aqueous cryoprotectant buffer for IM administration. The vaccine DP can be formulated at a concentration of 0.1 mg/mL RNA in 10 mM Tris buffer, 300 mM sucrose, pH 7.4.
Quadrivalent Influenza modRNA vaccine (qIRV) is a preservative-free, sterile dispersion of LNPs in aqueous cryoprotectant buffer for IM administration. The vaccine DP can be formulated at a concentration of 0.1 mg/mL RNA in 10 mM Tris buffer, 300 mM sucrose, pH 7.4.

In the bivalent COVID-19 vaccine component of the combination vaccine tested in the present Example, constructs each encode one of two SARS-CoV-2 spike protein antigens, the wildtype plus a variant matching a strain in circulation and recommended for primary or booster vaccination. In the qIRV component of the combination vaccines, modRNA constructs each encode one of four influenza HA antigens that match annually recommended vaccine components for cell-based or recombinant vaccines.

In some embodiments, both influenza and SARS COV-2 components of a combination vaccine can be matched to identified circulating strains. In some embodiments, this vaccine can offer protection against emerging SARS-CoV-2 variants and influenza strains recommended for vaccination for a given season, and thus decrease the number of vaccinations needed.

Planned Efficacy Evaluation: Combination COVID-19 & Influenza mRNA Vaccine

The Phase 1 substudy secondary immunogenicity objective is to describe immune responses elicited by combination qIRV (22/23)/bivalent BNT162b2 (Original/OMI BA.4/BA.5) vaccines at various dose level combinations. Secondary immunogenicity endpoints include HAI titer for 2022/2023 seasonal strains (2×A, 2×B) recommended by the World Health Organization (WHO) for recombinant or cell-based influenza vaccines, and SARS-CoV-2 neutralizing titers against reference (Wuhan) strain and Omicron BA.4/BA.5 sublineage. Sera are tested pre-vaccination and at 1, 4, and 8 weeks post vaccination to determine geometric mean titers (GMTs), geometric mean fold-rises (GMFRs), proportions of participants achieving seroconversion/seroresponse, and proportions of participants with HAI titers ≥1:40 for each strain or for all strains.

Note: influenza seroconversion is defined in the present study as HAI titer <1:10 prior to vaccination and ≥1:40 at the time point of interest, or HAI titer ≥1:10 pre-vaccination and a 4 fold rise post-vaccination. SARS-CoV-2 seroresponse is defined as achieving ≥4-fold rise from baseline (pre-vaccination) or, if the baseline measurement is below LLOQ, post-vaccination measure of ≥4×LLOQ.

Exploratory immunogenicity objectives can include describing immune responses elicited by the combination vaccines to heterologous influenza strains at various dose level combinations, evaluated as HAI titer for heterologous strains not matched to the WHO-recommended 2022/23 seasonal strains (2×A, 2×B). A vaccine-matched (antigenically similar) strain is defined in the present study as inducing ≤4-fold difference in HAI titer relative to a reference serum. Additionally, an exploratory immunogenicity objective is to describe the immune response to emerging SARS-CoV-2 variants of concern, based on SARS-CoV-2 neutralizing titers for Omicron sublineages and variants not already specified.

Phase 2/3 and Phase 3 substudies include analyses to demonstrate noninferiority of immune responses elicited by combination vaccines versus standard of care, with statistical criteria for immunogenicity endpoint analyses to be specified in the final statistical analysis plan.

Both the COVID-19 and influenza RNA vaccines characterized in this study can elicit a neutralizing antibody response. RNA vaccines can also induce a T cell response and innate responses can contribute to protection.

Immunogenicity, particularly influenza HA and SARS COV-2 spike-protein specific responses, can be an early marker of an appropriate immune response, supporting decision making on candidate and dose level selections, and provide evidence of noninferiority to an appropriate comparator vaccine to support future licensure.

### Geometric Mean Titers (GMTs)

GMTs can be calculated as the mean of assay results after making a logarithm transformation and exponentiating the mean to express results on the original scale.

Two-sided 95% CIs can be obtained by taking log transforms of assay results, calculating the 95% CI with reference to Student's t distribution, and exponentiating the confidence limits.

### Geometric Mean Fold Rises (GMFRs)

Fold rises can be defined as ratios of results after vaccination to results before vaccination. Calculation of fold rises is limited to participants with nonmissing values at both time points.

GMFRs can be calculated as the mean of the difference of logarithmically transformed assay results (later time point minus earlier time point) and exponentiating the mean. The associated 2-sided 95% CIs can be obtained by constructing CIs using Student's t distribution for the mean difference on the logarithm scale and exponentiating the confidence limits.

### Overview of Safety

Nonclinical studies from BNT162b2 and influenza modRNA programs support effectiveness of the vaccines at clinically tested dose levels up to a total of at least 100-μg modRNA.

Planned Safety Evaluation: Combination COVID-19 & Influenza mRNA Vaccine

Overall, potential risks to be considered for combination COVID-19 & influenza mRNA vaccines include those typically associated with all vaccines administered via IM injection, such as local reactions and systemic events that might arise from the active vaccine component (mRNA) or other components in the final drug product (LNPs).

### Benefit-Risk Assessment

A favorable benefit/risk profile in support of clinical development of combination COVID-19 & influenza mRNA vaccines can be based on: (1) safety and immunogenicity from ongoing clinical study of the bivalent BNT162b2 COVID-19 vaccine (the results of which are described in the previous Examples), (2) extensive real-world effectiveness and safety data for BNT162b2, (3) ongoing clinical study of qIRV, and (4) nonclinical studies of BNT162b2 and influenza modRNA vaccines.

Overall, potential risks to be considered for a combination COVID-19 & influenza mRNA vaccine include those typically associated with all vaccines administered via IM injection, such as local reactions and systemic events that might arise from the active vaccine component (mRNA) or other components in the final drug product (LNPs).

A combination RNA vaccine can address important public health needs in the ongoing SARS-CoV-2 pandemic outbreak and provides a long-term potential advantage to individuals in enabling them to receive one combined vaccine best matched to current circulating strains of each virus, so as to optimally protect against both influenza and COVID-19.

Collectively, the available clinical and nonclinical data support certain benefits of a combined COVID-19 and influenza RNA vaccine to outweigh the potential associated risks in healthy patients.

A primary objective of the Phase 1 Substudy A is to describe the safety and tolerability of the combination vaccine at different dose level combinations. Phase 2/3 and Phase 3 substudies include the same safety assessments. Safety evaluations include:
Reactogenicity recorded in an electronic diary (e-diary) up to 7 days after each vaccination
Adverse events (AEs) reported from first vaccination to 4 weeks after last vaccination
Serious AEs (SAEs) reported from first vaccination to 4 weeks after last vaccination

Reactogenicity includes prompted local reactions and systemic events recorded daily in an e-diary for a 7-day period after each dose. Local reactions include injection site redness, swelling, and pain. Systemic events include fever, fatigue, headache, chills, vomiting, diarrhea, new or worsened muscle pain, and new or worsened joint pain. Additional Phase 1 assessments are intended to identify abnormalities consistent with probable or possible myo/pericarditis, based on percentage of participants with abnormal troponin I laboratory values 2 days after the last vaccination or percentage of participants with new electrocardiogram (ECG) abnormalities 2 days and 1 week after vaccination.

### Certain Benefits and Risks

In some embodiments, the combination COVID-19 & influenza mRNA vaccine provide effective protection against influenza and COVID-19 illness through induction of neutralizing antibodies and T cell responses to the vaccine encoded viral antigens. Reactogenicity and AEs typically associated with IM administered vaccines may be observed and have been observed in nonclinical toxicology studies and clinical studies of the modRNA influenza and COVID-19 vaccines, developed on the same vaccine platform.

### Example 5: Flu/COVID modRNA LNP Animal Study Design

The objective of the experiment described in the present example was to measure the immunogenicity of combination vaccines containing a QIRV vaccine (quadrivalent influenza RNA)+COVID-19 bivalent RNA vaccine (WT+Omicron variant, e.g., WT+BA.4/5 Omicron) and to assess whether administering the vaccines in combination would interfere with immunogenicity of the encoded HA and/or S polypeptides (as compared to influenza or SARS-CoV-2 vaccines administered separately).

Data provided in the present Example demonstrate that, in some embodiments, a combination Flu+COVID-19 vaccine can elicit an immune response that is at least not reduced as compared to immune responses induced by standalone Flu and COVID-19 vaccines, and which may be comparable or even greater than that induced by standalone Flu and COVID-19 vaccines.

Approach: Mice were immunized with standalone or combo LNP_Flu and LNP_COVID-19 modRNA materials. Sera were collected at Day 21 post prime and at Day 42 (14 days post boost) and were evaluated by serology testing (Flu HAI & microneutralization (MNT), and SARS-CoV-2 neutralization assays). 10 mice were tested per group.

Significance: This study assessed the feasibility of a combo Flu+COVID-19 modRNA vaccine approach.

Material Needs: Flu Vaccine comparator=NH22-23 licensed comparators in-house (Fluad, Fluzone HD). In the present example, Fluad NH 22-23 and Fluzone HD were used, and compared to a quadrivalent modRNA vaccine encoding HA polypeptides of the following strains:
A/Wisconsin/588/2019 (H1N1)
A/Darwin/6/2021 (H3N2)
B/Austria/1359417/2021 (B-Victoria)
B/Phuket/3073/2013 (B-Yamagata)

Fluad and Fluzone HD comprised antigens from the following strains:
A/Victoria/2570/2019 IVR-215
A/Darwin/6/2021 IVR-227
B/Austria/1359417/2021 BVR-26
B/Phuket/3073/2013 BVR-1b

The present study used a bivalent modRNA COVID-19 vaccine, comprising BNT162b2 (encoding WT SARS-CoV-2 S protein) and BNT162b2 OMI (encoding a SARS-CoV-2 S protein comprising mutations characteristic of a BA.4/5 variant). Both RNAs in the bivalent COVID-19 vaccine encoded a prefusion stabilized S protein, comprising proline mutations at positions corresponding to residues 986 and 987 of SEQ ID NO: 1.

Table 49, below, summarizes certain experimental details of the present study.

| | | | | | | Vax | Bleed |
|---|---|---|---|---|---|---|---|
| Gp# | Mice | RNA DP Description | Dose (μg) | Dose Vol / Route | | (Day) | (Day) |
| 1 | 10 | Saline | — | 50 | μl / IM | 0, 28 | 21, 42 |
| 2 | 10 | Quadrivalent modFlu HA | 0.8 total | 50 | μl / IM | 0, 28 | 21, 42 |
| | | (4x HA premix¹) | (0.2 μg ea) | | | | |
| 3 | 10 | Bivalent modCOVID S (WT + | 0.8 total | 50 | μl / IM | 0, 28 | 21, 42 |
| | | Omicron BA.4/5_P2) | (0.4 μg ea.) | | | | |
| 4 | 10 | Quad modFlu and Bivalent | 1.6 ug total | 50 | μl / IM | 0, 28 | 21, 42 |
| | | (WT + Omicron | (0.2 μg each Flu | | | | |
| | | BA.4/5_P2) modCOVID - | HA + 0.4 each | | | | |
| | | (postmix¹) | COVID | | | | |
| | | | modRNA) | | | | |
| 5 | 10 | Quad modFlu and Bivalent | 0.8 ug total | 50 | μl / IM | 0, 28 | 21, 42 |
| | | (WT + Omicron | (0.1 μg each Flu | | | | |
| | | BA.4/5_P2) modCOVID | HA + 0.2 each | | | | |
| | | (postmix¹) | COVID | | | | |
| | | | modRNA) | | | | |
| 6 | 10 | Quad modFlu and Bivalent | 1.6 ug total | 50 μl / IM Flu | | 0, 28 | 21, 42 |
| | | (WT + Omicron | 0.8 (0.2 μg each | leg #1 + | | | |
| | | BA.4/5_P2) modCOVID | Flu HA) + 0.8 | 50 μl COVID / IM | | | |
| | | (separate admin in each | (0.4 each | leg #2 | | | |
| | | leg) | COVID | | | | |
| | | | modRNA) | | | | |
| 7 | 10 | Fluad (NH 22-23) + | 2.4 ug Fluad + | 20 μl Flu leg #1 + | | 0, 28 | 21, 42 |
| | | Bivalent (WT + Omicron | 0.8 μg Covid (0.4 | 50 μl/ IM COVID | | | |
| | | BA.4/5_P2) modCOVID | each COVID | leg#2 | | | |
| | | (separate admin in each | modRNA) | | | | |
| | | leg) | | | | | |
| 8 | 10 | Fluad (NH 22-23) | 2.4 μg | 20 | μl/IM | 0, 28 | 21, 42 |
| | | (60 μg/0.5 mL) | | | | | |
| 9 | 10 | Fluad (NH 22-23) | 2.4 μg (diluted) | 50 | μl/IM | 0, 28 | 21, 42 |
| | | (60 μg/0.5 mL) | | | | | |
| 10 | 10 | Fluzone HD (NH 22-23) - | 10.3 μg | 30 | μl/IM | 0, 28 | 21, 42 |

240 μg/0.7 mL

¹Postmix = formulate individual drug substances and mix together individual LNPs. Premix = combine drug substances and then co-formulate into LNP

Table 50, below, summarizes further details regarding the animals
to be studied in the present experiment.

| | |
|---|---|
| Animal species | Mice |
| Animal strain | Balb/c |
| Animal gender | Female |
| Animal vendor | Jackson Laboratory |
| Age at arrival | 5-7 weeks |
| Age at study start | 10-12 weeks |
| Animals in study | 100 |
| Vax prep | Sterile glass vials |
| Delivery | 0.3 mL insulin syringes |
| | 1/animal^{a} (groups 1-5, & 8-10) |
| | 2/animal^{a} (groups 6 & 7) |
| | Prime (groups 1-5 & 8-10) - left leg |
| | Boost (groups 1-5 & 8-10) - right leg |
| | Prime (groups 6 & 7) - leg #1 left leg, |
| | leg #2 right leg |
| | Boost (groups 6 & 7) - leg #1 right leg, |
| | leg #2 left leg |

^{a}1/animal indicates that a dose was administered as one injection. 2/animal indicates that a dose was administered as two injections.

TABLE 51
Study Schedule

| Day | Procedure |
|---|---|
| 0 | Vaccination #1 |
| 21 | Submandibular Bleed |
| 28 | Vaccination #2 |
| 42 | Terminal bleed (cardian puncture) |

On days 21 and 42, HIA and microneutralization titers (MNT) assays were performed on serum samples collected for groups 1, 2, and 4-10, and SARS-CoV-2 pVNT were collected for groups 1, 3-7.

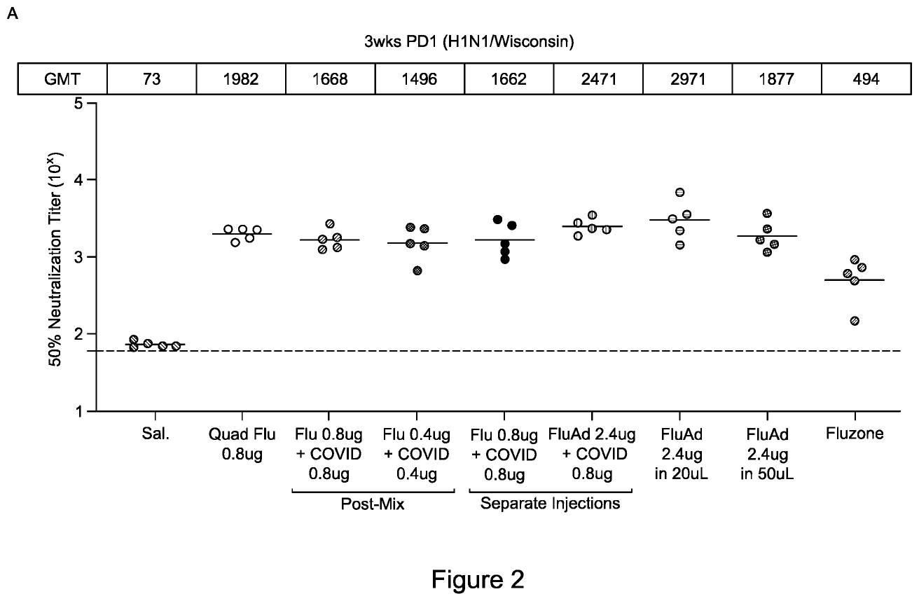

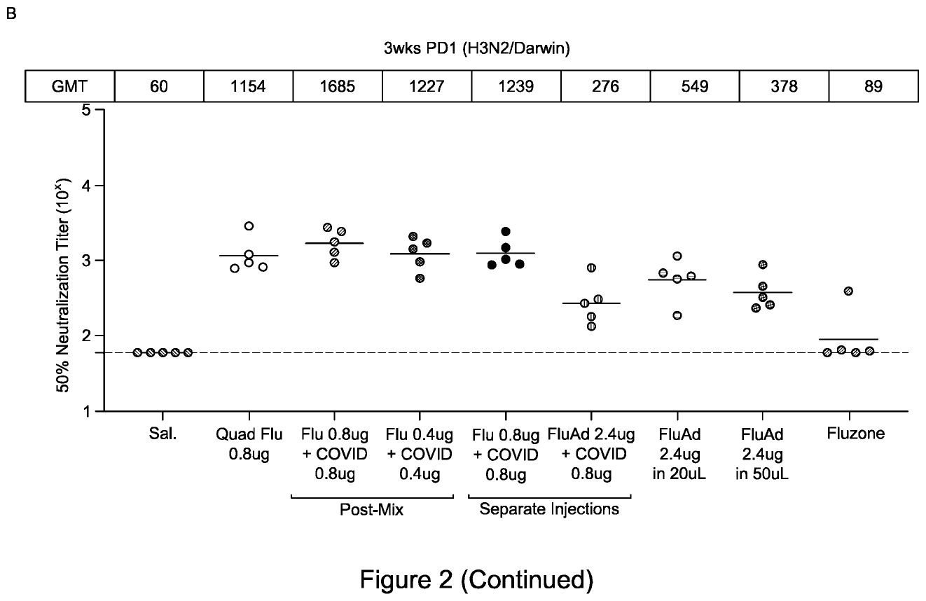

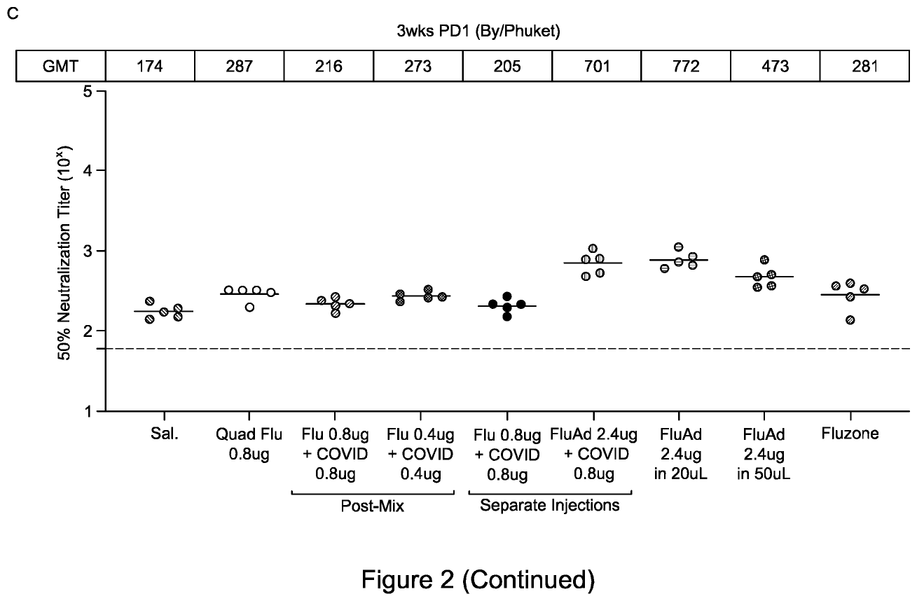

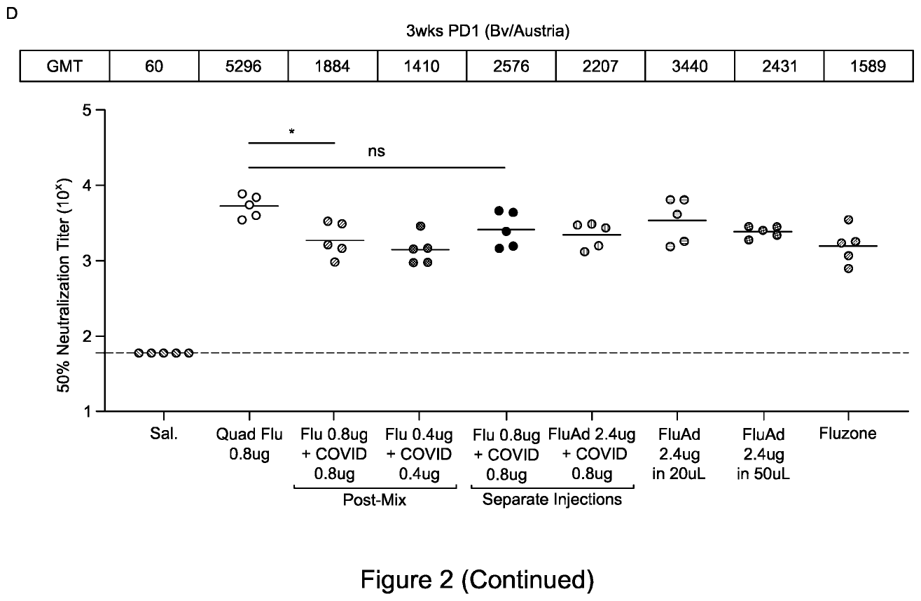

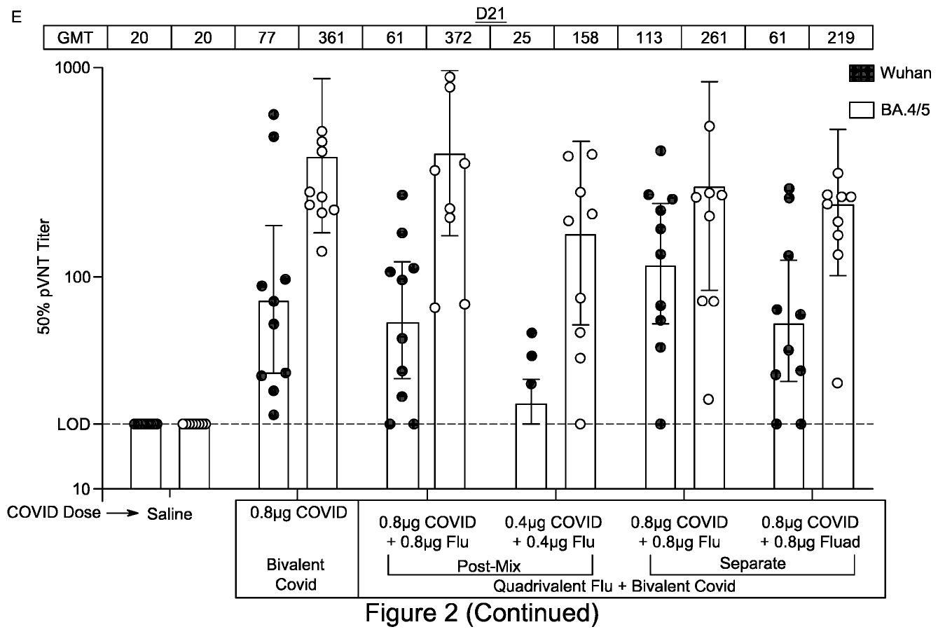

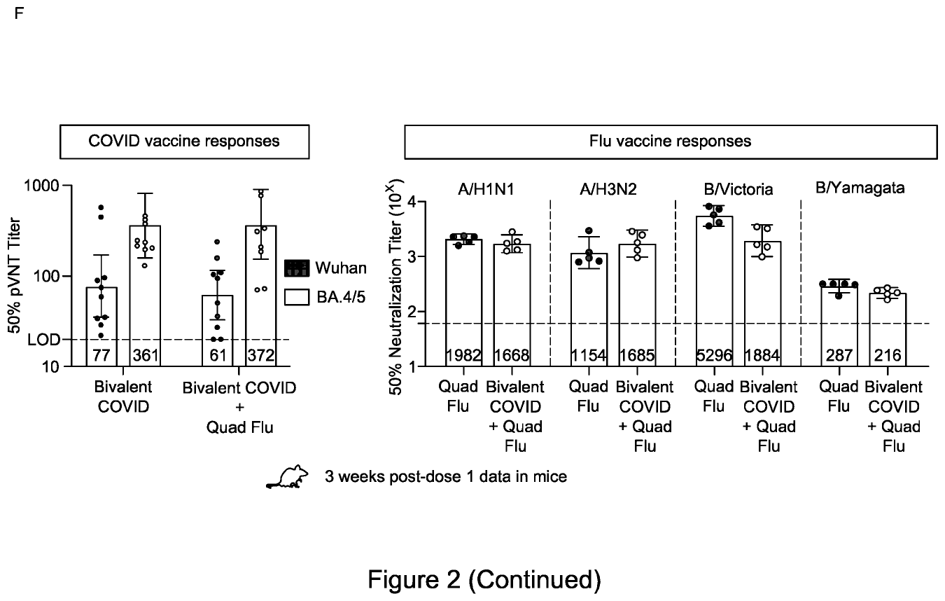

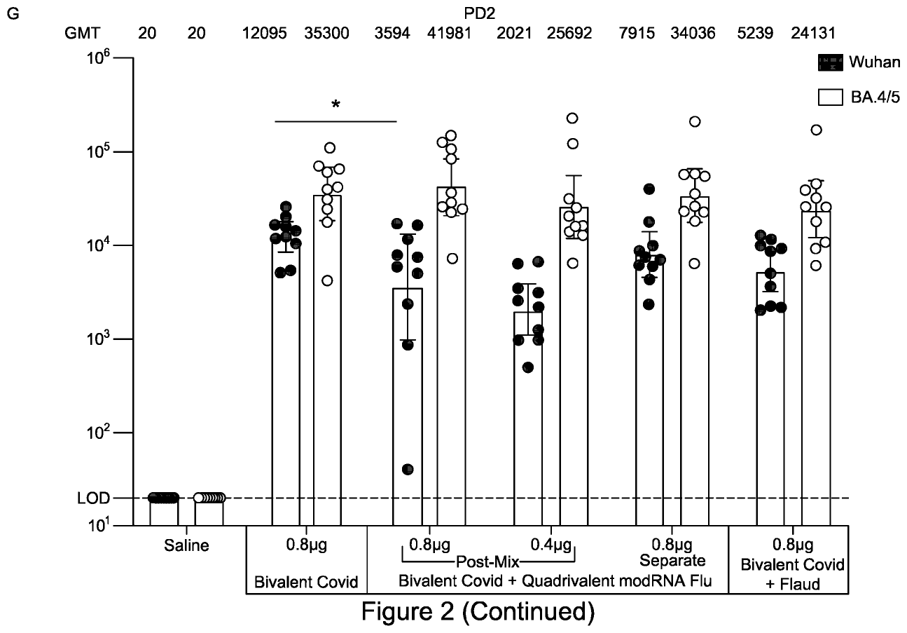

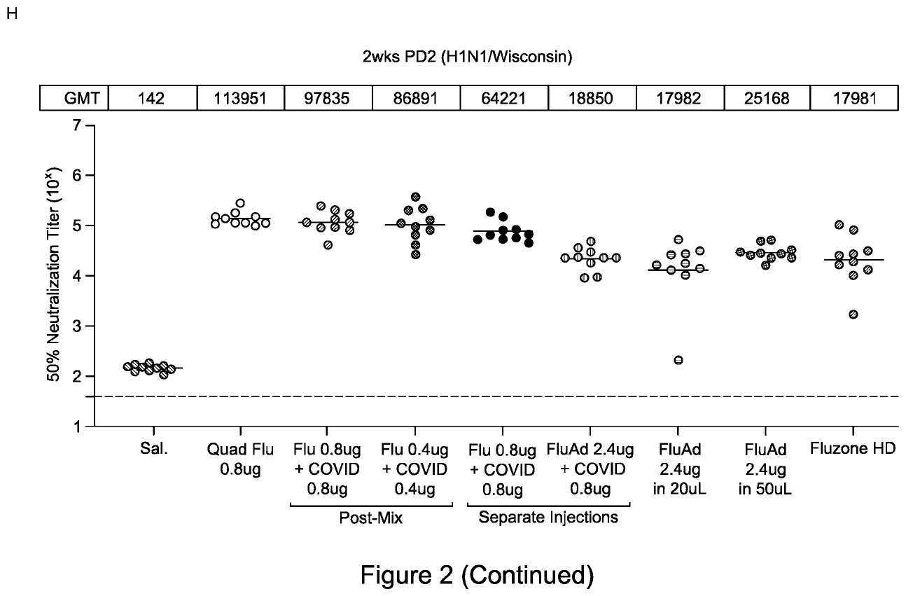

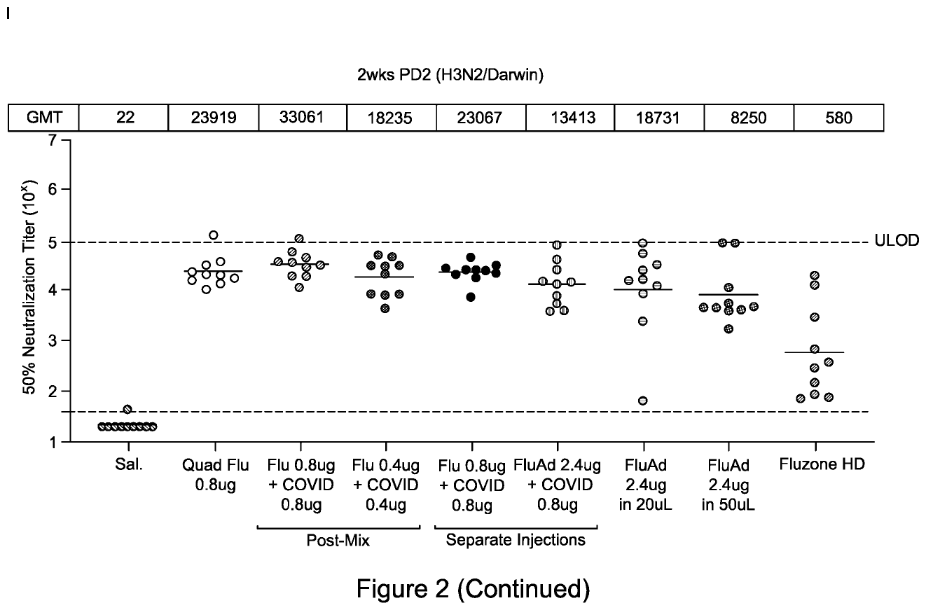

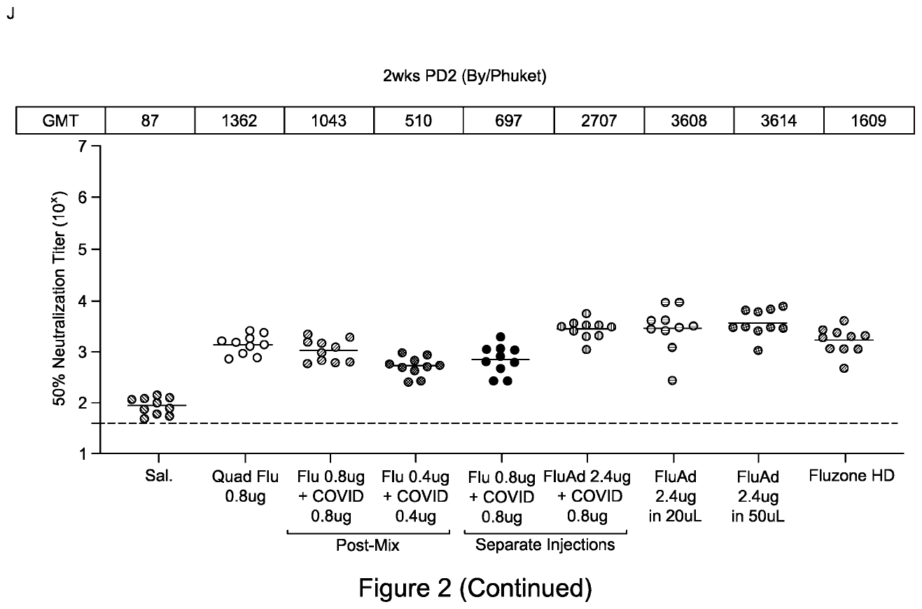

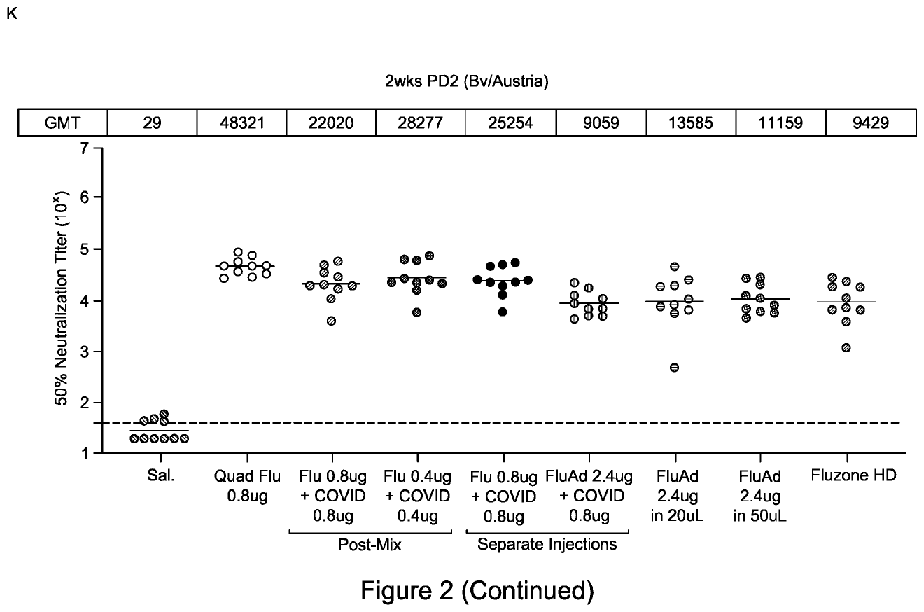

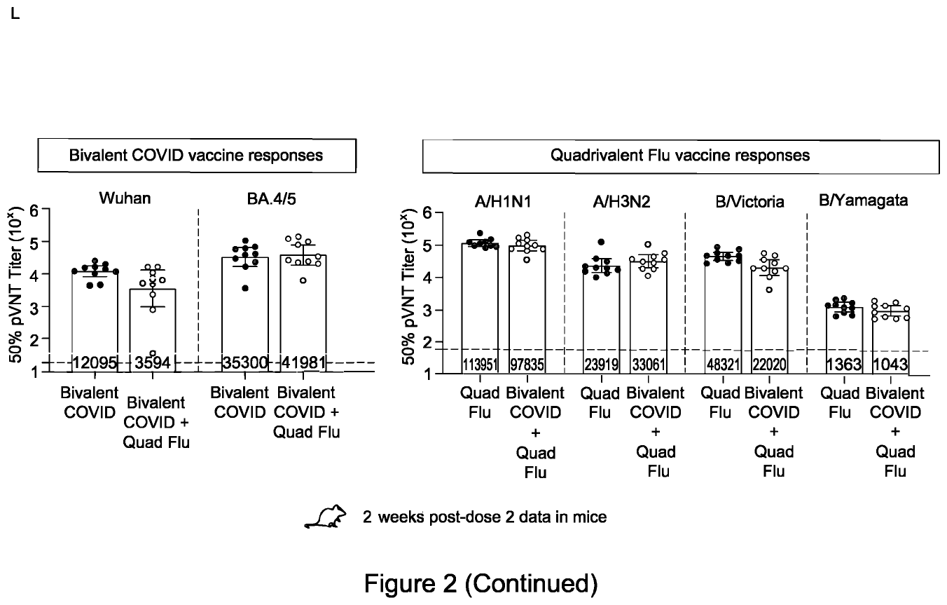

Post-Dose 1 (PD1) and Post-Dose 2 (PD2) results are shown in FIGS. 2(A)-(L). FIGS. 2(A)-(D) show MNT assay results for an H1N1/Wisconsin, H3N2/Darwin, B/Phuket, and B/Austria strain, respectively, 3 weeks after administering a first dose of a vaccine. As shown in the figures, no significant interference in immune response against A/Wisconsin and A/Darwin was observed after a first dose when the influenza and COVID-19 modRNA vaccines were administered in combination, while a slight interference against B/Austria was observed. B/Phuket titers were reduced PD₁ when the influenza and COVID modRNA vaccines were administered in combination, but titers were too low to determine whether an interference was observed.

FIGS. 2(H)-(K) show MNT neutralization results against H1N1/Wisconsin, H3N2/Darwin, By/Phuket, and Bv/Austria, respectively, 2 weeks after administering a second dose of a vaccine. As shown in the Figures, administering two doses of a COVID-19 and influenza modRNA combination produced similar neutralization responses as administering two doses of an influenza modRNA vaccine alone for H1N1/Wisconsin, H3N2/Darwin, and By/Phuket, while the combination was found to produce a slight decrease in titers for Bv/Austria. For all groups, neutralization titers were higher after a second dose of vaccine as compared to after a first dose.

FIG. 2(E) shows neutralization titers against a pseudovirus comprising a SARS-CoV-2 S protein of a Wuhan strain or a SARS-CoV-2 S protein comprising mutations characteristic of an Omicron BA.4/5 variant, in sera collected 3 weeks after administration of a first dose of a vaccine. As shown in the Figure, immunogenicity of a Bivalent COVID-19 vaccine was not affected when administered in combination with either a quadrivalent Influenza modRNA vaccine or Fluad.

FIG. 2(G) shows neutralization titers against a pseudovirus comprising a SARS-CoV-2 S protein of a Wuhan strain or a SARS-CoV-2 S protein comprising mutations characteristic of an Omicron BA.4/5 variant, in sera collected 2 weeks after administration of a second dose of a vaccine. Similar to post-dose 1, no or minimal interference was observed when the COVID-19 vaccine was administered in combination with the influenza vaccine, as compared to when it was administered alone. For all groups, administering a second dose of vaccine increased neutralization responses.

FIG. 2(F) summarizes the SARS-CoV-2 immune responses shown in FIGS. 2(A)-(E). As can be seen in the figure, no or minimal inference was seen in combination vaccines as compared to standalone vaccines.

### Example 6: Clinical and Non-Clinical Data for Influenza RNA Vaccine

The present Example describes data from clinical and non-clinical studies that characterize an investigational influenza modRNA vaccine intended to prevent disease caused by influenza viruses. The vaccines characterized in the present Example comprise up to 8 modRNAs, each encoding a single influenza HA or NA antigen. RNA modifications in the tested modRNAs blunt innate immune sensor activating capacity, thereby decreasing the rate of RNA degradation and promoting antigen expression. In the vaccines studied in the present example, modRNA is encapsulated in LNPs, which enable transfection of modRNA into host cells after intramuscular (IM) injection. The LNP formulation used in the present Example contains 2 functional lipids, ALC-0315 and ALC-0159, and 2 structural lipids, DSPC and cholesterol. Potency of modRNA vaccines can be improved by LNP encapsulation, which protects RNA from degradation by extracellular RNases. The influenza modRNA-LNP platform characterized in the present study is identical to the platform used in the SARS-CoV-2 vaccine, BNT162b2 (Comirnaty®), which was authorized for emergency use in the US on 11 Dec. 2020 for individuals ≥16 years of age and on 10 May 2021 for individuals ≥12 years of age.

### Summary of Non-Clinical Studies

Non-clinical studies summarized in the present Example demonstrated that influenza modRNA vaccine candidates are immunogenic in mice and rats. These in vitro and in vivo studies also established a mechanism-of-action for influenza modRNA vaccines—encoded influenza HA antigens were shown to induce an immune response characterized by both a strong functional antibody response and a Th1-type CD4+ and an IFNg+ CD8+ T-cell response. This mechanism of action is similar to that established for BNT162b2.

Immunogenicity studies in mice, benchmarked against a licensed, adjuvanted inactivated influenza vaccine, supported potential use of a multivalent influenza modRNA vaccine formulation to target 4 different influenza virus strains. Initial immunogenicity studies in mice of an octavalent HA/NA modRNA vaccine indicated no interference for influenza A strains and modest reductions of antibody responses for influenza B strains in comparison to monovalent control vaccines.

Biodistribution was assessed using luciferase expression as a surrogate reporter formulated using the same LNP-formulation as the influenza modRNA vaccines characterized in the present Example, with an identical lipid composition. After IM injection of LNP-formulated RNA encoding luciferase in BALB/c mice, luciferase expression was demonstrated at the site of injection 6 hours post dose and was not detected after 9 days. Luciferase was detected to a lesser extent in the liver; expression was present at 6 hours after injection and was not detected by 48 hours after injection. After IM administration of a radiolabeled LNP-mRNA with a comparable lipid composition to the influenza modRNA vaccine to rats, the percent of administered dose was also greatest at the injection site. Outside of the injection site, total recovery of radioactivity was greatest in the liver and much lower in the spleen, with very little recovery in the adrenal glands and ovaries.

The first-in-human (FIH) study of influenza modRNA vaccine was supported by established nonclinical safety of the modRNA-LNP platform and clinical safety of the BNT162b2 vaccine and vaccines encoding HA antigens. Nonclinical safety of the modRNA-LNP platform used with the influenza vaccines characterized in the present Example has been well-characterized in the COVID-19 vaccine program with a favorable safety profile demonstrated in 4 BNT162 modRNA vaccine candidates at up to 100 μg mRNA per dose for up to 3 doses in Wistar Han rats. The toxicology findings of all 4 BNT162 modRNA vaccine candidates were comparable, supporting the notion that vaccines with the same modRNA-LNP platform share equivalent safety profiles. The influenza modRNA vaccine characterized in the present study uses the same RNA manufacturing process and is in the same LNP formulation as in BNT62 vaccines.

In addition, the safety of the HA antigens that the modRNA encodes are well-documented in licensed vaccines and clinical trials with a large clinical safety data set.

Repeat-dose toxicity studies in rats with an influenza modRNA monovalent HA vaccine candidate (targeting the A/Wisconsin/588/2019 strain) or an influenza modRNA quadrivalent HA vaccine (Quad modRNA) targeting the A/Wisconsin/588/2019, A/Cambodia/e0826360/2020, B/Phuket/3073/2013, and B/Washington/02/2019 strains, confirmed that the vaccine candidates shared a similar toxicity profile to the BNT162 modRNA vaccine candidates. In addition, a GLP-compliant DART study with a modRNA quadrivalent HA vaccine candidate revealed no vaccine-related effects on fertility, gestation, or lactation in female rats or on embryo-fetal or postnatal survival, growth, or development in the F1 offspring.

Based on the nonclinical experience with modRNA-LNP platform derived from BNT162b2 vaccine candidates as well as the repeat-dose toxicity study results with the influenza modRNA monovalent or quadrivalent HA vaccine candidates, target organs in rats for this modality were the injection site, immune organs (draining and regional lymph nodes, spleen, and bone marrow), and liver. All findings were nonadverse and most were related to immune/inflammatory responses to the vaccine, including injection site erythema and edema and microscopic mixed cell inflammation, increased cellularity in draining lymph nodes and spleen, and increased hematopoiesis in the bone marrow and spleen. LNP uptake-associated hepatocellular vacuolation was of low severity and reversible without evidence of hepatocyte injury.

### Summary of Clinical Studies

Experience with BNT162b2 in clinical studies and post-approval use indicated that a modRNA-LNP platform has a good safety profile and the ability to induce immune responses.

Therefore, the nonclinical and clinical packages of BNT162b2, in addition to the nonclinical toxicology and immunogenicity data with influenza modRNA vaccine candidates, supported the clinical study evaluation of influenza modRNA vaccine candidates using a modRNA-LNP platform at up to 100 μg total RNA for a total of 3 IM administrations.

Safety and immunogenicity data, obtained up to 4 weeks following vaccination with monovalent, bivalent, or quadrivalent influenza modRNA vaccines also indicated that doses of 30 μg and 60 μg were safe and likely to elicit an immune response at least similar to licensed quadrivalent influenza vaccine. These data supported further clinical development, including the study of doses >30 μg. In some embodiments, commercialized influenza vaccines can be in a liquid formulation stable at 2° C. to 8° C. for ≥9 months, with dose level and number of doses to be determined in a Phase 1 dose-finding study.

One (1)- and 2-dose vaccination regimens were explored, including combinations of modRNA influenza vaccines with Fluzone HD.

Further studies on co-administration with other vaccines, including pneumococcal vaccine and COVID-19 vaccine, are planned, if annual booster doses of COVID-19 vaccine are found to be required in the future.

Influenza strains that were prevalent in the 2021/2022 flu season, and which are predicted to be prevalent in the 2022/2023 flu season are listed below

### 2021/2022 Northern Hemisphere Strains:

Wisconsin modRNA—A/WISCONSIN/588/2019 (H1N1)
Phuket modRNA—B/PHUKET/3073/2013 (B YAMAGATA)
Washington modRNA—B/WASHINGTON/02/2019 (B VICTORIA)
Cambodia modRNA—A/CAMBODIA/e0826360/2020 (H3N2)

### 2022/2023 Northern Hemisphere Strains:

Wisconsin modRNA—A/WISCONSIN/588/2019 (H1N1)
Darwin modRNA—A/Darwin/6/2021 (H3N2)
Phuket modRNA—B/PHUKET/3073/2013 (B YAMAGATA)
Austria modRNA—B/AUSTRIA/1359417/2021 (B VICTORIA)

### 2023 Southern Hemisphere Strains:

Sydney modRNA—A/Sydney/5/2021 (H1N1)
Darwin modRNA—A/Darwin/6/2021 (H3N2)
Phuket modRNA—B/PHUKET/3073/2013 (B YAMAGATA)
Austria modRNA—B/AUSTRIA/1359417/2021 (B VICTORIA)

Four monovalent drug products (DPs) and two quadrivalent DPs were tested (listed below). Additional DPs may be produced (e.g., DPs adapted to match an influenza strain that is prevalent and/or predicted-to-be prevalent in a relevant location).
1. Monovalent: Wisconsin (PF-07829855) DP
2. Monovalent: Phuket (PF-07836259) DP
3. Monovalent: Washington (PF-07836261) DP
4. Monovalent: Cambodia (PF-07836258) DP
5. Quadrivalent: Wisconsin (PF-07829855), Phuket (PF-07836259), Washington (PF-07836261), Cambodia (PF-07836258) DP
6. Quadrivalent: Wisconsin (PF-07829855), Phuket (PF-07836259), Darwin (PF-07871853), Austria (PF-07872963) DP

The influenza modRNA vaccines discussed in the present Example comprise modRNA encoding strain-specific full length, codon-optimized HA envelope glycoprotein (the protein responsible for viral binding to target cells and mediating cell entry) and/or NA (the protein responsible for enabling a virus to be released from a host cell). ModRNA influenza vaccine DP characterized in the present study was a preservative-free, sterile dispersion of LNPs in aqueous cryoprotectant buffer for IM administration. The vaccine DP was formulated at 0.1 mg/ml RNA in 10 mM Tris buffer, 300 mM sucrose, pH 7.4 as a single-dose vial with 0.5 mL/vial fill volume, and 0.3 mL nominal volume.

### Drug Substance

Specific construct (i.e., Wisconsin modRNA, Phuket modRNA, Washington modRNA, Cambodia modRNA, Darwin modRNA, and Austria modRNA) drug substance materials were the only active ingredient(s) used in combination to manufacture the quadrivalent DPs discussed in the present Example. Drug substance was formulated in 10 mM HEPES buffer, 0.1 mM EDTA at pH 7.0 and stored at −20±5° C. in HDPE bottles EVA flexible containers.

### Excipients

The excipients Tromethamine (Tris base) and Tris Hydrochloride (HCl) were present in DP.

### Phase 1/2 Study

For monovalent dosing, influenza RNA vaccines can be dosed in the range of 3.75 to 30 μg per dose with an injection volume of 0.3 mL. Except for the 30-μg dose, use of the formulations described in the present Example required dilution with sterile 0.9% sodium chloride (normal saline). The 4 dose levels were:
3.75 μg
7.5 μg
15 μg
30 μg

A bivalent vaccine was dosed by mixing 2 monovalent vaccines in a total delivered volume of 0.3 mL. Dosing ranges (total RNA) and ratios of RNAs in bivalent vaccines were:

$$15 μg at 1 : 1 ( 7.5 μg + 7.5 μg or either A / A or A / B )$$

$$30 μg at 1 : 1 ( 15 μg + 15 μg or either A / A or A / B )$$

$$60 μg at 1 : 1 ( 30 μg + 30 μg of A / A )$$

$$22.5 μg at 1 : 2 ( 7.5 μg A + 15 μg B )$$

$$18.75 μg at 1 : 4 ( 3.75 μg A + 15 μg B )$$

Immunogenicity in Mice of a Multivalent Influenza modRNA Vaccine

Current licensed seasonal influenza vaccines are designed to protect against up to 4 different influenza viruses, including 2 influenza A viruses (H1N1 and H3N2 subtypes) and 2 influenza B viruses (B Yamagata and B Victoria lineages). To evaluate the feasibility of a multivalent formulation of a modRNA influenza vaccine, modRNAs encoding 4 different HA proteins and 4 different NA proteins were generated. Immune responses elicited by mice vaccinated with LNP-formulated modRNA encoding a single strain-specific HA or NA were compared to groups vaccinated with an octavalent HA/NA modRNA formulation. Octavalent formulation methods were compared by separately formulating each modRNA expressing HA or NA in LNPs and then mixing the 8 LNPs together in equal ratios, or by pre-mixing the 8 modRNAs followed by a single co-formulation in LNPs. A licensed, adjuvanted trivalent inactivated influenza vaccine (FluAd®, Seqirus) was included as a benchmark in the study.

BALB/c mice were immunized IM with 2 μg of each HA- and NA-expressing modRNA either as a monovalent or octavalent vaccine formulation in LNPs on Days 0 and 28. Robust antibody and T-cell responses were elicited by LNP-formulated modRNA to all HA and NA components, at levels similar to or greater than the licensed vaccine comparator. Similar HAI and neutralizing responses were observed on Day 49 (21 days after the second boost) for individual HA and octavalent formulations for influenza A strains; however, some interference was observed with B strains in the octavalent formulation. Antibodies measured against NA showed a similar trend as HA. Similar CD8+ and CD4+ T-cell responses were observed for monovalent HA and octavalent formulations for influenza strains, with some interference also noted for the B strains. In summary, HA/NA octavalent RNA vaccine formulations elicited similar antibody responses and T-cell responses as matched monovalent HA formulations for H1N1 and H3N2 strains, but reduced responses for HA of influenza B viruses. These initial immunogenicity studies in mice of an octavalent HA/NA mRNA vaccine suggest modest reductions of antibody responses in comparison to monovalent control vaccines. No difference in immunogenicity was observed with the 2 different multivalent formulation methods.

These data were obtained with influenza modRNA vaccine candidates encoding the HA and NA proteins derived from virus strains recommended for the 2018-2019 Northern hemisphere influenza season and formulated with LNPs that are related, but not identical, to those that are used in the clinical study. These initial mouse immunogenicity data support use of a multivalent modRNA formulation and are confirmed pre-clinically with the influenza modRNA vaccines against the 2020-2021 Northern hemisphere strains. These data also demonstrate the potential to supplement or combine an influenza modRNA vaccine expressing HA proteins with modRNA expressing NA proteins. NA may serve as a desirable vaccine antigen due to evidence that NA plays a role in reducing disease severity and inducing cross-protection.

Influenza modRNA Quadrivalent HA Vaccine—Repeated Dose Toxicity

In a 17-day, repeat-dose GLP-compliant toxicity study, IM administration of an influenza Quad modRNA HA vaccine (30 μg/dose, administered once every 2 weeks for a total of 2 doses [Days 1 and 15]), targeting the A/Wisconsin/588/2019, A/Cambodia/e0826360/2020, B/Phuket/3073/2013, and B/Washington/02/2019 strains, was tolerated without any evidence of systemic toxicity. Nonadverse changes were observed and consistent with an expected response to vaccines and/or secondary to inflammation similar to those described for monovalent modRNA, except that hepatocyte vacuolation was interpreted to reflect hepatocyte uptake of the lipid in the LNP formulation. Partial to full recovery of all findings occurred by the end of the recovery phase except for globulin and A/G ratio. Rats administered an influenza Quad modRNA HA vaccine exhibited a hemagglutinin inhibition response. At the end of the dosing phase, nonadverse Flu Quad HA modRNA vaccine-related microscopic observations occurred at the injection site (inflammation and edema), which correlated with the observation of dark color macroscopically, in the draining and inguinal lymph nodes (increased cellularity of plasma cells and germinal centers), spleen (increased cellularity of hematopoietic cells and germinal centers) which correlated with enlarged spleens and higher spleen weights, bone marrow (increased cellularity of hematopoietic cells), and liver (periportal hepatocyte vacuolation). At the end of the recovery phase full recovery of findings occurred, except for partial recovery of the macroscopic finding of enlarged draining lymph nodes and microscopic findings of inflammation at the injection site, and increased cellularity of plasma cells and germinal centers in the draining and inguinal lymph nodes.

During the dosing phase, nonadverse Flu Quad HA modRNA vaccine-related lower reticulocyte and platelet counts, and higher WBC, neutrophil, monocyte, eosinophil, and LUC, hypersegmented neutrophils in peripheral blood smears, prolonged PT and higher fibrinogen, lower albumin and A/G ratio and higher globulin, lower cholesterol, and higher A1AGP and A2M were observed. All clinical pathology findings were fully recovered except for higher globulin and lower A/G ratio which did not recover.

Flu Quad HA modRNA vaccine-related nonadverse lower mean body weight and decreased mean food consumption occurred transiently and recovered by the end of the first week of the study, and higher mean body temperature occurred following each dose.

Administration of Flu Quad HA modRNA resulted in a hemagglutinin inhibition response to each of the 4 influenza strains in the vaccine at the end of the dosing and recovery phase of study. The mean titers for each of the 4 influenza strains was higher at the end of the recovery phase than at the end of the dosing phase. At the end of the recovery phase, the mean titers from highest to lowest for each strain was A/Wisconsin/588/2019>B/Phuket/3073/2013>B/Washington/02/2019>A/Cambodia/e0826360/2020.

Influenza modRNA Vaccine Reproductive and Developmental Toxicity

The effects of Flu Quad HA modRNA on fertility, embryo-fetal (including an evaluation of teratogenicity), and pre- and postnatal development in the pregnant and lactating Wistar han rat was evaluated. Flu Quad HA modRNA was administered intramuscularly into the quadriceps muscle of female rats at a dose of 30 μg on 2 occasions (21 and 14 days) before cohabitation and on 2 occasions (GD 9 and 20) during the gestation phase.

There were no Flu Quad HA modRNA vaccine-related clinical signs or effects on body weights or food consumption. There were no vaccine-related macroscopic observations. There were no vaccine-related effects on estrous cycling, mating, fertility and pregnancy indices, or any ovarian, uterine, or litter parameter, including F1 survival, growth or external, visceral, skeletal malformations or variations, or effects on pinna detachment, eye opening, auditory startle response, or pupil constriction in the F1 pups. Flu Quad HA modRNA vaccine administration resulted in vaccine-induced hemagglutinin titers to each of the 4 influenza strains at all timepoints (DS 22, GD 21, and LD 21) in the F0 females and in their F1 offspring (fetuses or pups).

In conclusion, intramuscular administration of Flu Quad HA modRNA vaccine before and during gestation to Wistar Han female rats was tolerated with no indication of maternal systemic toxicity. There were no Flu Quad HA modRNA vaccine-related effects on fertility, gestation, or lactation in female rats or on embryo-fetal or postnatal survival, growth, or development in the F1 offspring.

### Effects in Human

### Substudy A (Phase 1)

A randomized, observer-blinded (sponsor-unblinded) substudy was performed to evaluate the safety and immunogenicity of monovalent influenza RNA vaccines (A/Wisconsin or B/Phuket in the present Example) at 4 dose levels (3.75, 7.5, 15, or 30 μg), bivalent RNA vaccines (A/Wisconsin and B/Phuket in the present Example) at 4 dose-level combinations, and qIRV at a dose level of 7.5 μg per HA for 2 A strains (A/Wisconsin and A/Cambodia in the present Example) and 2 B strains (B/Phuket and B/Washington in the present Example). Additionally, Substudy A described the immune response elicited by licensed QIV (Fluzone HD in the present Example) following prior receipt of a modRNA vaccine, to assess potential priming of the immune response, and the immune response elicited by mIRV (A/Wisconsin or B/Phuket in the present Example) following prior receipt of a licensed QIV, to assess if the immune response following QIV may be enhanced.

Based upon preliminary immunogenicity data 1 week following Vaccination 1, it was decided not to expand enrollment from 255 to 615 participants as planned in the study protocol. Therefore, 254 participants were enrolled in Substudy A.

All participants enrolled in Substudy A have reached 4 weeks after Vaccination 1.

The safety and immunogenicity data available to-date from Substudy A (i.e., at 1 and 4 weeks following vaccination) are described below.

Based on these data, the protocol in Substudy B was designed to include increased doses of influenza modRNA vaccine, with a total dose not exceeding 100 μg. This is supported by an acceptable tolerability profile for a single dose up to 30 μg and opportunity to increase the immune response.

### Safety

The population for an analysis of safety included participants who received vaccine and had any safety data. A total of 60 participants (15 participants per dose level/dose level combination) each were randomized to the mIRV-A, mIRV-B, and bIRV-A and -B groups, and 15 participants were randomized to a qIRV group. All the participants received the vaccination, except for 1 participant in the mIRV-A 30-μg group. One participant in the mIRV-B 15-μg group discontinued after vaccination due to protocol deviation but continued in the study for safety follow-up. Numerically, there were more female participants across all vaccine groups with the exception of the mIRV-A vaccine group, where there were more male participants. The majority of the participants were White. The mean age at vaccination across all vaccination groups was approximately 70 years of age.

### Local Reactions

Local reactions within 7 days after vaccination for each vaccine group are described below. A licensed QIV was used as a comparator.

mIRV-A

Among participants receiving mIRV-A, 25 participants reported mild-to-moderate pain at the injection site. No redness or swelling was reported at any mIRV-A dose level. A slight dose-related increase in reactogenicity (pain at the injection site) was noted.

mIRV-B

Among participants receiving mIRV-B, 23 participants reported any local reaction. Pain at the injection site (n=22) was the most frequently reported local reaction followed by redness (n=4) and swelling (n=3). All the local reactions were mild to moderate in severity and the median duration was 1 to 4 days. No dose-related increase in reactogenicity was apparent.

bIRV—A and B

Among participants receiving bIRV-A and B, 27 participants reported any local reaction. Pain at the injection site (n=27) was the most frequently reported local reaction, followed by redness and swelling (n=4 each). All the local reactions were mild to moderate in severity and the median duration was 1 to 5 days. No dose-related increase in reactogenicity was apparent.

qIRV

Among participants receiving qIRV, 13 participants reported any local reaction. Pain at the injection site (n=13) was the most frequently reported local reaction, followed by swelling (n=1). Both of these local reactions were mild to moderate in severity, and the median duration was 2 days.

### Systemic Events

Systemic events within 7 days after vaccination for each vaccine group are described below. A licensed QIV was used as a comparator.

mIRV-A

Among participants receiving mIRV-A, 19 participants reported any systemic event. One participant from the 30-μg vaccine group reported a fever of ≥38.0° C. to 38.4° C. for 1 day. Fatigue (n=14) was the most frequently reported systemic event, followed by headache (n=8), new or worsened muscle pain (n=5), chills and diarrhea (n=3 each). All of the systemic events were mild to moderate in severity and the median duration was 1 to 6 days.

mIRV-B

Among participants receiving mIRV-B, 18 participants reported any systemic event. One participant from the 3.75-μg vaccine group reported a fever of >38.4° C. to 38.9° C. Fatigue (n=13) was the most frequently reported systemic event, followed by headache (n=12), chills and new or worsened muscle pain (n=6 each), new or worsened joint pain (n=4), and diarrhea (n=2). All systemic events were mild to moderate in severity and the median duration was 1 to 8 days.

bIRV—A and B

Among participants receiving bIRV-A and B, 27 participants reported any systemic event. Fatigue (n=22) was the most frequently reported systemic event, followed by new or worsened muscle pain (n=11), headache (n=10), new or worsened joint pain (n=8), chills and diarrhea (n=5 each), and vomiting (n=1). Of note, no systemic event of fever was reported. All systemic events were mild to moderate in severity and the median duration was 1 to 5.5 days.

qIRV

Among participants receiving qIRV, 7 participants reported any systemic event. Fatigue (n=6) was the most frequently reported systemic event, followed by headache (n=5), chills (n=2), and new or worsened muscle pain and new or worsened joint pain (n=1 each). All systemic events were mild to moderate in severity and the median duration was 1 to 5 days.

### Adverse Events

AEs within 4 weeks after vaccination for each vaccine group are described below. A licensed QIV was used as a comparator. No deaths were reported in the study.

Of note, no ECG or safety laboratory abnormalities of clinical concern were noted following vaccination.

mIRV-A

Among participants receiving mIRV-A, 7 participants reported at least 1 occurrence of any AE. One participant each from the 3.75-μg (malaise) and 7.5-μg (pruritus) vaccine groups reported AEs related to the vaccine. One participant from the 15-μg vaccine group reported severe viral gastroenteritis 8 days following vaccination which was judged unrelated to study intervention.

mIRV-B

Among participants receiving mIRV-B, 12 participants reported at least 1 occurrence of any AE. One participant from the 30-μg vaccine group reported an AE (C-reactive protein increased) related to the vaccine. One participant from the 3.75-μg vaccine group reported an SAE of hip fracture, which was not related to the vaccine.

bIRV—A and B

Among participants receiving bIRV-A and B, 15 participants reported at least 1 occurrence of any AE. Three participants from the A 7.5-μg+B 15-μg vaccine group reported AEs (C-reactive protein increased [n=2] and ophthalmic migraine [n=1]) related to the vaccine. One participant from the A 3.75-μg+B 15-μg vaccine group reported an SAE of rib fracture, which was not related to the vaccine.

qIRV

Among the participants receiving qIRV, 2 participants reported at least 1 occurrence of any AE (injection site pruritus [n=1] and haemoglobin decreased [n=1]) both of which were assessed as related to the vaccine.

### Quadrivalent Immunogenicity

In participants who received qIRV, HAI GMTs and GMFRs for all strains at 4 weeks following vaccination were elevated relative to baseline, comparably to participants who received licensed QIV.

The proportion of participants achieving seroconversion for A/Wisconsin and A/Cambodia at 4 weeks following administration of qIRV was higher than in participants who received licensed QIV, with the difference in the proportion of participants achieving seroconversion for these strains being 20.0% and 13.3% respectively. The proportion of participants achieving seroconversion for B/Phuket 4 weeks following administration of qIRV was 14.3% (1.8, 42.8), compared to 26.7% (7.8, 55.1) of participants having received licensed QIV; while 20.0% of participants achieved seroconversion for B/Washington 4 weeks following administration of either qIRV or licensed QIV.

The proportion of participants achieving seroconversion 1 and 4 weeks following administration of qIRV is shown in Table 52.

TABLE 52
Difference in Proportion of Participants Achieving HAI Seroconversion
for Each Strain at Each Time Point After Vaccination - qIRV - Licensed
QIV - Evaluable Immunogenicity Population

| | | Vaccine Group (as Randomized) | | |
|---|---|---|---|---|
| Sampling | | qIRV^{a} N^{c} | qIRV - Licensed QIV N^{c} | |
| Time Point^{b} | Strain | n^{d} (%) (95% CIe) | nd (%) (95% CIe) | Difference % |
| 4 Weeks | B/Washington | 15 1 (6.7) (0.2, 31.9) | 15 0 (0.0) (0.0, 21.8) | 20.0 |
| | A/Wisconsin | 15 13 (86.7) (59.5, 98.3) | 15 10 (66.7) (38.4, 88.2) | 13.3 |
| | A/Cambodia | 15 8 (53.3) (26.6, 78.7) | 15 6 (40.0) (16.3, 67.7) | −12.4 |
| | B/Phuket | 14 2 (14.3) (1.8, 42.8) | 15 4 (26.7) (7.8, 55.1 | 6.7 |
| | B/Washington | 15 3 (20.0) (4.3, 48.1) | 15 3 (20.0) (4.3, 48.1) | 0.0 |

Abbreviations: HAI = hemagglutination inhibition assay; modRNA = nucleoside-modified messenger ribonucleic acid; qIRV = quadrivalent influenza modRNA vaccine; QIV = quadrivalent influenza vaccine.
Note:
Seroconversion is defined as an HAI titer <1:10 prior to vaccination and ≥1:40 at the time point of interest, or an HAI titer of ≥1:10 prior to vaccination with a 4-fold rise at the time point of interest.
Note:
The paired samples collected at Day 1 prior to vaccination were selected for seroconversion calculation.
^{a}qIRV at a dose level of 7.5 μg for each of the following encoded strains: A/Wisconsin, A/Cambodia, B/Phuket, and B/Washington.
^{b}Protocol-specified timing for blood sample collection.
^{c}N = number of participants with valid and determinate assay results for the specified assay both before vaccination and at the dose/sampling time point.
^{d}n = Number of participants with valid and determinate assay results for the specified assay achieving HAI seroconversion at the dose/sampling time point

### Substudy B (Phase 1/2) for Elderly Subjects

The following section describes a randomized, single-blinded (sponsor-unblinded) substudy to evaluate the safety and immunogenicity of the vaccination schedules detailed below. Participants 65 to 85 years of age were randomized to one of the following vaccination schedules and were blinded to which 1-visit or 2-visit vaccination schedules they receive:

### 2-Visit Schedules

2 Doses of qIRV encoding 2 A strains and 2 B strains at a dose level of 7.5 μg per strain, administered 21 days apart.
2 Doses of licensed QIV, administered 21 days apart (as a control group).
A dose of licensed QIV following by a dose of bIRV encoding 2 A strains at a dose level of either 15 or 30 μg per strain, administered 21 days apart.

### 1-Visit Schedules

A dose of licensed QIV administered concurrently in the opposite arm with bIRV encoding 2 A strains at a dose level of either 15 or 30 μg per strain.
A dose of bIRV encoding 2 A strains at a dose level of 15 μg per strain administered concurrently in the opposite arm with bIRV encoding 2 B strains at a dose level of 15 μg per strain.
A dose of qIRV encoding 2 A strains and 2 B strains at the following dose level combinations:
   15 μg per strain
   7.5 μg per A strain, and 22.5 μg per B strain
   7.5 μg per A strain, and 37.5 μg per B strain
   A dose of licensed QIV (as a control group).

### Initial Enrolment

### 2-Visit Schedule

A total of 63 participants were randomized to a 2-visit schedule of which 1 participant from qIRV 30 μg/qIRV 30 μg dosing group was not vaccinated. Therefore, a total of 62 participants received 1 dose of study intervention and 58 participants received 2 doses of study intervention. Fourteen participants were vaccinated with the first dose of study intervention in qIRV 30 μg/qIRV 30 μg group and 16 participants each were vaccinated in Licensed QIV/bIRV A 30 μg group, Licensed QIV/bIRV A 60 μg, and Licensed QIV/licensed QIV group. The mean age across the 2-visit groups was approximately 70 years of age.

### 1-Visit Schedule

A total of 64 participants were randomized to a 1-visit schedule and all participants received 1 dose of study intervention. One participant was randomized to Licensed QIV+bIRV A 30 μg (1-visit schedule) but received Licensed QIV/bIRV A 30 μg (2-visit schedule) due to a medication error. Sixteen participants each were vaccinated in the Licensed QIV+bIRV A 30 μg group, Licensed QIV+bIRV A 60 μg group, qIRV 60 μg group, and Licensed QIV group. The mean age across the 1-visit group was approximately 70 years of age.

### Local Reactions

Local reactions within 7 days after vaccination for each vaccine group are described below. A licensed QIV was used as a comparator.

### 2-Visit Schedule

Among participants who received a first dose of study intervention, 24 participants reported any local reaction. Pain at injection site (n=23) was the most frequently reported local reaction followed by swelling (n=3) and redness (n=2). Except 1 participant reporting severe redness, all other local reactions were mild to moderate in severity and the median duration was 1 to 3 days.

Among participants who received second doses of study intervention, 17 participants reported any local reaction. Pain at injection site (n=17) was the most frequently reported local reaction followed by redness and swelling (n=3 each). All the local reactions were mild to moderate in severity and the median duration was 1 to 4 days.

### 1-Visit Schedule

A total of 27 participants reported local reactions. Pain at injection site (n=26) was the most frequently reported local reaction followed by redness (n=4) and swelling (n=3). All local reactions were mild to moderate in severity and the median duration was 1 to 7 days.

### Systemic Events

Systemic events within 7 days after the vaccination for each vaccine group are described below. A licensed QIV was used as a comparator.

### 2-Visit Schedule

Among participants who received the first dose of study intervention, 19 participants reported any systemic event. One participant reported fever of >38.4° C. to 38.9° C., which lasted for 1 day. Fatigue (n=17) was the most frequently reported systemic event followed by headache (n=12), chills (n=7), new or worsened joint pain (n=6), diarrhea (n=4), and new or worsened muscle pain (n=3). All of the reported systemic events were mild to moderate in severity and the median duration was 1 to 8 days.

Among the participants who received a second dose of study intervention, 21 participants reported any systemic event. One participant reported fever of >38.0° C. to 38.4° C., which lasted for 1 day. Fatigue and headache (n=11 each) were the most frequently reported systemic events followed by chills, new or worsened muscle pain (n=8 each), new or worsened joint pain (n=5), diarrhea (n=3), and vomiting (n=1). All of the systemic events were mild to moderate in severity and the median duration was 1 to 7 days.

### 1-Visit Schedule

A total of 25 participants reported any system event after the single dose of vaccination. One participant reported fever of >38.0° C. to 38.4° C. Fatigue (n=19) was the most frequently reported systemic event followed by headache (n=15), new or worsened muscle pain (n=13), chills (n=7), new or worsened joint pain (n=5), and vomiting (n=1). All of the systemic events were mild to moderate in severity and the median duration was 1 to 4 days.

### Adverse Events (Initial Enrolment)

AEs within 4 weeks after vaccination are described below. A licensed QIV was used as a comparator. No deaths were reported in the study.

### 2-Visit Schedule

Among the participants who received 2 doses of study intervention, 6 participants reported at least 1 AE. These were AEs of arthralgia, arthritis, chest pain, electrocardiogram ST segment depression, fatigue, skin laceration, supraventricular tachycardia, and tooth abscess. One participant from Licensed QIV/Licensed QIV group reported an AE of arthralgia, which was related to the vaccine.

Routinely scheduled post-vaccination ECGs showed 8 new changes distributed among the four 2-dose groups, these included first degree AV block, ectopic atrial rhythm, premature ventricular complexes in 2 participants, ST depression, supraventricular tachycardia, and T wave flattening in 2 participants. Of these, only the ST depression, which was in an asymptomatic participant, was initially deemed clinically significant, and yielded follow up ECGs, which were deemed not significant; this finding was ultimately deemed unrelated to vaccine administration. There were no abnormal troponin values noted post-vaccination.

### 1-Visit Schedule

A total of 3 participants reported at least 1 AE. These were cholelithiasis, cough, medical device site reaction, and rhinorrhea. One participant reported an SAE of cholelithiasis in Licensed QIV+bIRV A 30 μg vaccine group. Routinely scheduled post-vaccination ECGs showed 6 new changes distributed among three of the one-dose groups (participants in the licensed QIV+bIRV A 60 μg group had no ECG changes identified); these included left anterior fascicular block, premature ventricular complexes, right bundle branch block, sinus bradycardia in 2 participants, and T wave flattening, none of which were deemed clinically significant.

### Immunogenicity

A dose-dependent increase in HAI GMTs was observed 4 weeks following administration of vaccine, which was also reflected in the GMFR relative to baseline at this timepoint.

Regarding seroconversion 4 weeks following administration of last dose of vaccine:
For A/Wisconsin, the proportion of participants achieving seroconversion following either 1 or 2 doses of qIRV, at a total dose level of 30 μg per dose, was higher than the proportion of participants achieving seroconversion following either 1 or 2 doses of QIV. Additionally, for A/Wisconsin, 4 weeks following QIV administered with bIRV encoding 2 A strains at a total dose of 30 or 60 μg (15 or 30 μg per strain), either concurrently, or 21 days apart, the proportion of participants achieving seroconversion also appeared to be higher than the proportion of participants achieving seroconversion following either 1 or 2 doses of QIV (see Table 47). For A/Cambodia, the proportion of participants achieving seroconversion following 1 dose of qIRV, at 30 μg per dose, was higher than the proportion of participants achieving seroconversion following QIV; this response was similar to QIV after the second dose. Additionally for A/Cambodia, the proportion of participants achieving seroconversion 4 weeks following QIV administered concurrently with bIRV encoding 2 A strains at a total dose of 30 or 60 μg (15 or 30 μg per strain) appeared higher than QIV alone.
For B/Phuket and B/Washington, the proportion of participants achieving seroconversion 4 weeks following study intervention in all groups, including 1 and 2 dose QIV recipients, was lower compared to the proportion of participants achieving seroconversion against A/Wisconsin and A/Cambodia. For B/Phuket and B/Washington, the proportion of participants achieving seroconversion 4 weeks following either 1 or 2 doses of qIRV, at a total dose level of 30 μg per dose, or following QIV administered with bIRV encoding 2 A strains at a total dose level of either 30 or 60 μg (15 or 30 μg per strain), either concurrently, or 21 days apart, appeared to be generally less than or equal to the proportion of participants achieving seroconversion following either 1 or 2 doses of QIV (see Table 53).

TABLE 53
Proportion of Participants Achieving HAI Seroconversion for Each
Strain at Each Timepoint - Evaluable Immunogenicity Population

| | | Vaccine Group (as Randomized) | | | |
|---|---|---|---|---|---|
| | | 2-Visit Schedule | | | |
| | | | | Licensed | |
| | | | | QIV/ | Licensed |
| | | | Licensed | bIRV A | QIV/ |
| | | qIRV (30 μg)/ | QIV/ bIRV | (60 μg) | Licensed |
| Sampling | | qIRV (30 μg) | A (30 μg) | N^{b} n^{c} | QIV |
| Time | | N^{b} n^{c} (%) | N^{b} n^{c} (%) | (%) (95% | N^{b} n^{c} (%) |
| Point^{a} | Strain | (95% CI^{d}) | (95% CI^{d}) | CI^{d})) | (95% CI^{d}) |
| Prior to | A/Wisconsin | 10 9 (90.0) | 11 7 (63.6) | 11 7 (63.6) | 13 7 (53.8) |
| Vaccination | | (55.5, 99.7) | (30.8, 89.1) | (30.8, 89.1) | (25.1, 80.8) |
| 2 | A/Cambodia | 10 7 (70.0) | 11 5 (45.5) | 11 5 (45.5) | 13 7 (53.8) |
| | | (34.8, 93.3) | (16.7, 76.6) | (16.7, 76.6) | (25.1, 80.8) |
| | B/Phuket | 10 1 (10.0) | 11 3 (27.3) | 11 5 (45.5) | 13 7 (53.8) |
| | | (0.3, 44.5) | (6.0, 61.0) | (16.7, 76.6) | (25.1, 80.8) |
| | B/Washington | 10 2 (20.0) | 11 4 (36.4) | 11 4 (36.4) | 13 6 (46.2) |
| | | (2.5, 55.6) | (10.9, 69.2) | (10.9, 69.2) | (19.2, 74.9) |
| 1 Week | A/Wisconsin | 10 9 (90.0) | 11 9 (81.8) | 11 10 | 13 7 (53.8) |
| after | | (55.5, 99.7) | (48.2, 97.7) | (90.9) | (25.1, 80.8) |
| the last | | | | (58.7, 99.8) | |
| vaccination | A/Cambodia | 10 8 (80.0) | 11 8 (72.7) | 11 7 (63.6) | 13 6 (46.2) |
| | | (44.4, 97.5) | (39.0, 94.0) | (30.8, 89.1) | (19.2, 74.9) |
| | B/Phuket | 10 4 (40.0) | 11 3 (27.3) | 11 5 (45.5) | 13 7 (53.8) |
| | | (12.2, 73.8) | (6.0, 61.0) | (16.7, 76.6) | (25.1, 80.8) |
| | B/Washington | 10 2 (20.0) | 11 3 (27.3) | 11 4 (36.4) | 13 6 (46.2) |
| | | (2.5, 55.6) | (6.0, 61.0) | (10.9, 69.2) | (19.2, 74.9) |
| 4 Weeks | A/Wisconsin | 10 9 (90.0) | 11 9 (81.8) | 11 10 | 13 5 (38.5) |
| after | | (55.5, 99.7) | (48.2, 97.7) | (90.9) | (13.9, 68.4) |
| the last | | | | (58.7, 99.8) | |
| vaccination | A/Cambodia | 10 4 (40.0) | 11 3 (27.3) | 11 5 (45.5) | 13 6 (46.2) |
| | | (12.2, 73.8) | (6.0, 61.0) | (16.7, 76.6) | (19.2, 74.9) |
| | B/Phuket | 10 5 (50.0) | 11 3 (27.3) | 11 3 (27.3) | 13 7 (53.8) |
| | | (18.7, 81.3) | (6.0, 61.0) | (6.0, 61.0) | (25.1, 80.8) |
| | B/Washington | 10 3 (30.0) | 11 2 (18.2) | 11 2 (18.2) | 13 5 (38.5) |
| | | (6.7, 65.2) | (2.3, 51.8) | (2.3, 51.8) | (13.9, 68.4) |

| | | Vaccine Group (as Randomized) | | | |
|---|---|---|---|---|---|
| | | 1-Visit Schedule | | | |
| | | Licensed | Licensed | | |
| | | QIV + | QIV + | | |
| | | bIRV A | bIRV A | qIRV | Licensed |
| | | (30 μg) | (60 μg) N^{b} | (60 μg) | QIV |
| Sampling | | N^{b} n^{c} (%) | n^{c} (%) | N^{b} | N^{b} |
| Time | | (95% | (95% | n^{c} (%) | n^{c} (%) |
| Point^{a} | Strain | CI^{d}) | CI^{d}) | (95% CI^{d}) | (95% CI^{d}) |
| Prior to | A/Wisconsin | NA | NA | NA | NA |
| Vaccination | A/Cambodia | NA | NA | NA | NA |
| 2 | B/Phuket | NA | NA | NA | NA |
| | B/Washington | NA | NA | NA | NA |
| 1 Week | A/Wisconsin | 12 9 (75.0) | 13 7 (53.8) | 15 10 (66.7) | 16 6 (37.5) |
| after | | (42.8, | (25.1, | (38.4, 88.2) | (15.2, 64.6) |
| the last | | 94.5) | 80.8) | | |
| vaccination | A/Cambodia | 12 2 (16.7) | 13 3 | 15 4 (26.7) | 16 4 (25.0) |
| | | (2.1, 48.4) | (23.1) | (7.8, 55.1) | (7.3, 52.4) |
| | | | (5.0, 53.8) | | |
| | B/Phuket | 12 4 (33.3) | 13 4 | 15 1 (6.7) (0.2, | 16 3 (18.8) |
| | | (9.9, 65.1) | (30.8) | 31.9) | (4.0, 45.6) |
| | | | (9.1, 61.4) | | |
| | B/Washington | 12 3 (25.0) | 13 4 | 15 0 (0.0) (0.0, | 16 3 (18.8) |
| | | (5.5, 57.2) | (30.8) | 21.8) | (4.0, 45.6) |
| | | | (9.1, 61.4) | | |
| 4 Weeks | A/Wisconsin | 12 7 (58.3) | 13 7 | 15 12 (80.0) | 16 6 (37.5) |
| after | | (27.7, 84.8) | (53.8) | (51.9, 95.7) | (15.2, 64.6) |
| the last | | | (25.1, | | |
| vaccination | | | 80.8) | | |
| | A/Cambodia | 12 7 (58.3) | 13 8 | 15 11 (73.3) | 16 4 (25.0) |
| | | (27.7, 84.8) | (61.5) | (44.9, 92.2) | (7.3, 52.4) |
| | | | (31.6, | | |
| | | | 86.1) | | |
| | B/Phuket | 12 6 (50.0) | 13 5 | 15 5 (33.3) | 16 2 (12.5) |
| | | (21.1, 78.9) | (38.5) | (11.8, 61.6) | (1.6, 38.3) |
| | | | (13.9, | | |
| | | | 68.4) | | |
| | B/Washington | 12 3 (25.0) | 13 5 | 15 3 (20.0) | 16 5 (31.3) |
| | | (5.5, 57.2) | (38.5) | (4.3, 48.1) | (11.0, 58.7) |
| | | | (13.9, | | |
| | | | 68.4) | | |

A/Cambodia.
Note:
The paired samples collected at Day 1 prior to Vaccination 1 were selected for seroconversion calculation.
Note:
Seroconversion is defined as an HAI titer <1:10 prior to first vaccination and ≥1:40 at the time point of interest, or an HAI titer of ≥1:10 prior to first vaccination with a 4-fold rise at the time point of interest.
^{a}Protocol-specified timing for blood sample collection.
^{b}N = number of participants with valid and determinate assay results for the specified assay both before first vaccination and at the dose/sampling time point.
^{c}n = Number of participants with valid and determinate assay results for the specified assay achieving HAI seroconversion at the dose/sampling time point.
^{d}Exact 2-sided CI based on the Clopper and Pearson method.

### Expanded Enrolment

### Local Reactions

Among the participants who received qIRV 30 μg, 55 participants reported any local reaction. Pain at injection site (n=50) was the most frequently reported local reaction followed by swelling (n=12) and redness (n=4). Except 1 participant reporting severe swelling, all other local reactions were mild to moderate in severity and the median duration was 1 to 2 days.

Among the participants who received qIRV 60 μg, 65 participants reported any local reaction. Pain at injection site (n=63) was the most frequently reported local reaction followed by swelling (n=12) and redness (n=9). Except 2 participants reporting severe pain at injection site, all other local reactions were mild to moderate in severity and the median duration was 1 to 2 days.

### Systemic Events

Among the participants who received qIRV 30 μg, 51 participants reported any systemic event. Four participants reported fever of ≥38.0° C. for 1 day. Fatigue (n=38) was the most frequently reported systemic event followed by headache (n=24), new or worsened muscle pain (n=19), new or worsened joint pain (n=15), chills (n=8), and diarrhea (n=5). One participant each reported severe fatigue, headache, and new or worsened joint pain. All other systemic events were mild to moderate in severity. The median duration was 1 day.

Among the participants who received qIRV 60 μg, 63 participants reported any systemic event. Seven participants reported fever of ≥38.0° C. for 1 day. Fatigue (n=49) was the most frequently reported systemic event followed by headache (n=32), new or worsened muscle pain (n=25), chills (n=21), new or worsened joint pain (n=20), diarrhea (n=5), and vomiting (n=3). Two participants reported severe fatigue, 1 participant each reported severe headache, chills, new or worsened muscle pain, and new or worsened joint pain. All other systemic events were mild to moderate in severity. The median duration was 1 to 1.5 days.

### Adverse Events

AEs within 4 weeks after vaccination for each vaccine group are described below. No SAEs or deaths were reported.

Among the participants who received qIRV 30 μg, a total of 8 participants reported any AE. The most frequently reported AE (≥2 participants) was injection site pain reported by 2 participants. Three participants reported AEs of diarrhea, hemorrhage subcutaneous, injection site pain, injection site pruritus, which were assessed as related to the study intervention.

Among the participants who received qIRV 60 μg, a total of 11 participants reported any AE. One participant reported severe AE. Three participants reported AEs of injection site pain, myocarditis, pericarditis, tinnitus, troponin abnormal, which were assessed as related to the study intervention. One participant had the events of myocarditis, pericarditis, and troponin abnormal.

The events of troponin abnormality, pericarditis, and myocarditis occurred in a 66-year-old African-American female with a medical history of hypertension, Chronic Obstructive

### Immunogenicity

A dose-dependent increase in HAI GMTs was observed 4 weeks following administration of vaccine.

For A/Wisconsin and A/Cambodia, the proportion of participants achieving seroconversion 4 weeks following qIRV 30 μg and qIRV 60 μg was higher than the proportion of participants achieving seroconversion following licensed QIV (Table 54).

For B/Phuket and B/Washington, the proportion of participants achieving seroconversion 4 weeks following qIRV 30 μg and qIRV 60 μg was lower compared to the proportion of participants achieving seroconversion following licensed QIV (Table 54).

TABLE 54
Proportion of Participants Achieving HAI Seroconversion for Each
Strain at Each Timepoint - Evaluable Immunogenicity Population
Vaccine Group (as Randomized)

| Sampling | | qIRV (30 μg) N^{b} | qIRV (60 μg) N^{b} | Licensed QIV N^{b} |
|---|---|---|---|---|
| Time Point^{a} | Strain | n^{c} (%) (95% CI^{d}) | n^{c} (%) (95% CI^{d}) | n^{c} (%) (95% CI^{d}) |
| 1 Week after | A/Wisconsin | 100 38 (38.0) | 107 48 (44.9) | 107 41 (38.3) |
| vaccination | | (28.5, 48.3) | (35.2, 54.8) | (29.1, 48.2) |
| | A/Cambodia | 98 21 (21.4) | 106 33 (31.1) | 107 23 (21.5) |
| | | (13.8, 30.9) | (22.5, 40.9) | (14.1, 30.5) |
| | B/Phuket | 99 8 (8.1) | 108 13 (12.0) | 107 31 (29.0) |
| | | (3.6, 15.3) | (6.6, 19.7) | (20.6, 38.5) |
| | B/Washington | 100 3 (3.0) | 108 8 (7.4) | 107 32 (29.9) |
| | | (0.6, 8.5) | (3.3, 14.1) | (21.4, 39.5) |
| 4 Weeks after | A/Wisconsin | 102 56 (54.9) | 108 67 (62.0) | 107 51 (47.7) |
| vaccination | | (44.7, 64.8) | (52.2, 71.2) | (37.9, 57.5) |
| | A/Cambodia | 101 42 (41.6) | 106 61 (57.5) | 107 31 (29.0) |
| | | (31.9, 51.8) | (47.6, 67.1) | (20.6, 38.5) |
| | B/Phuket | 101 12 (11.9) | 109 22 (20.2) | 107 34 (31.8) |
| | | (6.3, 19.8) | (13.1, 28.9) | (23.1, 41.5) |
| | B/Washington | 100 6 (6.0) | 109 13 (11.9) | 106 35 (33.0) |
| | | (2.2, 12.6) | (6.5, 19.5) | (24.2, 42.8) |

Abbreviations: HAI = hemagglutination inhibition; modRNA = modified RNA; qIRV = quadrivalent influenza modRNA vaccine; QIV = quadrivalent influenza vaccine.
Note:
qIRV at a dose level of 7.5 μg or 15 μg (total dose level is 30 μg or 60 μg respectively) for each of the following encoded strains: A/Wisconsin, A/Cambodia, B/Phuket, and B/Washington.
Note:
Seroconversion is defined as an HAI titer <1:10 prior to first vaccination and ≥1:40 at the time point of interest, or an HAI titer of ≥1:10 prior to first vaccination with a 4-fold rise at the time point of interest.
^{a}Protocol-specified timing for blood sample collection.
^{b}N = number of participants with valid and determinate assay results for the specified assay both before first vaccination and at the dose/sampling time point.
^{c}n = Number of participants with valid and determinate assay results for the specified assay achieving HAI seroconversion at the dose/sampling time point.
^{d}Exact 2-sided CI based on the Clopper and Pearson method.

TABLE 55
Difference in Proportion of Participants Achieving HAI Seroconversion for Each
Strain at 4 Weeks After Vaccination - Evaluable Immunogenicity Population

| | Vaccine Group (as Randomized) | | | | |
|---|---|---|---|---|---|
| | qIRV (30 μg) | qIRV (60 μg) | Licensed | Difference^{d} % (95% CI^{e}) | |
| | N^{a} n^{b} (%) | N^{a} n^{b} (%) | QIV N^{a} n^{b} (%) | qIRV (30 μg) | qIRV (60 μg) |
| Strain | (95% CI^{c}) | (95% CI^{c}) | (95% CI^{c}) | vs. Licensed QIV | vs. Licensed QIV |
| A/Wisconsin | 102 56 (54.9) | 108 67 (62.0) | 107 51 (47.7) | 7.2 | 14.4 |
| | (44.7, 64.8) | (52.2, 71.2) | (37.9, 57.5) | (−6.3, 20.5) | (1.0, 27.2) |
| A/Cambodia | 101 42 (41.6) | 106 61 (57.5) | 107 31 (29.0) | 12.6 | 28.6 |
| | (31.9, 51.8) | (47.6, 67.1) | (20.6, 38.5) | (−0.4, 25.3) | (15.4, 40.8) |
| B/Phuket | 101 12 (11.9) | 109 22 (20.2) | 107 34 (31.8) | −19.9 | −11.6 |
| | (6.3, 19.8) | (13.1, 28.9) | (23.1, 41.5) | (−30.7, −8.9) | (−23.2, 0.1) |
| B/Washington | 100 6 (6.0) | 109 13 (11.9) | 106 35 (33.0) | −27.0 | −21.1 |
| | (2.2, 12.6) | (6.5, 19.5) | (24.2, 42.8) | (−37.3, −16.9) | (−31.9, −10.2) |

Abbreviations: HAI = hemagglutination inhibition; modRNA = modified RNA; qIRV = quadrivalent influenza modRNA vaccine; QIV = quadrivalent influenza vaccine.
Note:
qIRV at a dose level of 7.5 μg or 15 μg (total dose level is 30 μg or 60 μg respectively) for each of the following encoded strains: A/Wisconsin, A/Cambodia, B/Phuket, and B/Washington.
Note:
Seroconversion is defined as an HAI titer <1:10 prior to first vaccination and ≥1:40 at the time point of interest, or an HAI titer of ≥1:10 prior to first vaccination with a 4-fold rise at the time point of interest.
^{a}N = number of participants with valid and determinate assay results for the specified assay both before first vaccination and at the dose/sampling time point.
^{b}n = Number of participants with valid and determinate assay results for the specified assay achieving HAI seroconversion at the dose/sampling time point.
^{c}Exact 2-sided CI based on the Clopper and Pearson method. d. Difference in proportions of participants achieving seroconversion, active vaccine groups -Licensed QIV as reference, expressed as a percentage.
^{e}2-Sided CI based on the Miettinen and Nurminen method for the difference in proportions (active vaccine groups - Licensed QIV) expressed as a percentage.

TABLE 56
Proportion of Participants Achieving HAI Seroconversion for All
Strains at Each Timepoint - Evaluable Immunogenicity Population

| | Vaccine Group (as Randomized) | | |
|---|---|---|---|
| Sampling | qIRV (30 μg) N^{b} n^{c} | qIRV (60 μg) N^{b} n^{c} | Licensed QIV N^{b} n^{c} |
| Time Point^{a} | (%) (95% CI^{d}) | (%) (95% CI^{d}) | (%) (95% CI^{d}) |
| 1 Week after | 97 0 (0.0) | 105 3 (2.9) | 107 11 (10.3) |
| vaccination | (0.0, 3.7) | (0.6, 8.1) | (5.2, 17.7) |
| 4 Weeks after | 98 3 (3.1) | 105 8 (7.6) | 106 14 (13.2) |
| vaccination | (0.6, 8.7) | (3.3, 14.5) | (7.4, 21.2) |

Abbreviations: HAI = hemagglutination inhibition; modRNA = modified RNA; qIRV = quadrivalent influenza modRNA vaccine; QIV = quadrivalent influenza vaccine.
Note:
qIRV at a dose level of 7.5 μg or 15 μg (total dose level is 30 μg or 60 μg respectively) for each of the following encoded strains: A/Wisconsin, A/Cambodia, B/Phuket, and B/Washington.
Note:
Seroconversion is defined as an HAI titer <1:10 prior to first vaccination and ≥1:40 at the time point of interest, or an HAI titer of ≥1:10 prior to first vaccination with a 4-fold rise at the time point of interest.
^{a}Protocol-specified timing for blood sample collection.
^{b}N = number of participants with valid and determinate assay results for all strains both before first vaccination and at the dose/sampling time point.
^{c}n = Number of participants with valid and determinate assay results for all strains achieving HAI seroconversion at the dose/sampling time point.
^{d}Exact 2-sided CI based on the Clopper and Pearson method.

### Summary of Data and Guidance for the Investigator

### Posology and Method of Administration

Influenza modRNA vaccine can be a suspension for injection containing RNA encapsulated in LNPs; the LNP formulation can comprise 2 functional lipids, ALC-0315 and ALC-01592, and 2 structural lipids, DSPC (1,2-distearoyl-sn-glycero-3-phosphocholine) and cholesterol. During each influenza season, 1 or 2 doses totaling less than 100 μg may be studied in individuals ≥18 years of age.

Influenza modRNA vaccine can be administered via IM injection into the deltoid region of the upper arm. The current injection volume is 0.3 mL or 0.5 mL.

### Special Warnings and Precautions for Use

### Concurrent Illness

Vaccination should be postponed in individuals suffering from acute severe febrile illness or acute infection. The presence of a minor infection and/or low-grade fever should not delay vaccination.

### Thrombocytopenia and Coagulation Disorders

As with other IM injections, the vaccine should be given with caution in individuals receiving anticoagulant therapy or those with thrombocytopenia or any coagulation disorder (such as hemophilia) because bleeding or bruising may occur following an IM administration in these individuals.

### Myocarditis and Pericarditis

Very rare cases of myocarditis and pericarditis have been reported following vaccination with mRNA COVID-19 vaccines. Typically, the cases have occurred more often in male adolescents and young adults after the second dose of the vaccine within several days after vaccination. These are generally mild cases and individuals tend to recover within a short time following standard treatment and rest. Patients can usually return to their normal daily activities after their symptoms improve. They should speak with their doctor about returning to exercise or sports. Healthcare professionals should be alerted to the signs and symptoms of myocarditis and pericarditis in vaccine recipients. Information is not yet available about potential long-term sequelae.

The mechanism by which mRNA COVID-19 vaccines could trigger cases of myocarditis and pericarditis has not been elucidated. If it relates to the modRNA or lipid nanoparticle formulation, rather than the encoded antigen(s), it is plausible that the same risk, risk factors, and prognosis may exist for influenza modRNA vaccines.

### Immunocompromised Individuals

The immunogenicity and efficacy of influenza modRNA vaccine may be lower in immunosuppressed individuals.

### Example 7: Clinical Trial to Study the Coadministration RSV/Covid/Influenza Vaccines

The present Example describes a clinical study performed to characterize an LNP-RNA vaccine and recombinant protein vaccine administered in combination (in the present Example, a bivalent SARS-CoV-2 vaccine (Wuhan+BA.4/5 BNT162b2) and a bivalent RSV vaccine (RSVpreF)). The purpose of the clinical trial summarized in the present Example was to characterize the safety and immunological effects of a combined vaccine for RSV and COVID-19, when administered with (i) a seasonal flu vaccine or (ii) alone. The data described in the present Example demonstrates that, when the two or three vaccines are co-administered, neutralization titers induced against each of the targeted infectious agents (SARS-CoV-2 Wuhan and encoded variants and RSV subtypes) are not inferior to neutralization titers induced by the vaccines administered alone.

### Design Study

Eligibility Criteria: Subjects meeting the following criteria were included in the study

### Inclusion Criteria:

Male or female participants ≥65 years of age at Visit 1 (Day 1, corresponding to the day interventions/treatments were administered);
Willing and able to comply with all scheduled visits, investigational plan, laboratory tests, lifestyle considerations, and other study procedures;
Healthy participants as determined by medical history, physical examination (if required), and clinical judgment of the investigator to be eligible for inclusion in the study;
Previously received at least 3 prior US authorized mRNA COVID 19 vaccines, with the last dose being an updated (bivalent) vaccine given at least 150 days before Day 1; and

### Exclusion Criteria:

A confirmed diagnosis of COVID 19, RSV infection, or influenza within 120 days of Day 1;
History of severe adverse reaction associated with any vaccine and/or severe allergic reaction (e.g., anaphylaxis) to any component of the study intervention(s);
Immunocompromised individuals with known or suspected immunodeficiency, as determined by history and/or laboratory/physical examination;
Bleeding diathesis or condition associated with prolonged bleeding that would, in the opinion of the investigator, contraindicate intramuscular injection;
Allergy to egg proteins (egg or egg products) or chicken proteins;
Any medical or psychiatric condition including recent (within the past year) or active suicidal ideation/behavior or laboratory abnormality that may increase the risk of study participation or, in the investigator's judgment, would have made the participant inappropriate for the study.
Receipt of chronic systemic treatment with known immunosuppressant medications (including cytotoxic agents or systemic corticosteroids), or radiotherapy, within 60 days before enrollment through conclusion of the study;
Receipt of blood/plasma products, immunoglobulin, or monoclonal antibodies, from 60 days before study intervention administration, or planned receipt throughout the study;
Receipt of any RSV vaccine at any time prior to enrollment, or planned receipt throughout the study;
Receipt of any influenza vaccine within 120 days before study enrollment;
Participation in other studies involving a study intervention within 28 days before randomization. Anticipated participation in other studies within 28 days after receipt of study intervention in this study.

Subjects meeting the above eligibility criteria were randomly divided into groups and administered a therapy as described in the below table.

| Group | N | Injection 1 (left arm) | Injection 2 (right arm) | Injection 3 (right arm) |
|---|---|---|---|---|
| 1 | 150 | [RSVpreF + BNT162b2] | QIV (Fluzone HD) | N/A |
| 2 | 150 | [RSVpreF + BNT162b2] | Placebo | N/A |
| 3 | 150 | BNT162b2 | Placebo | N/A |
| 4 | 150 | RSVpreF | Placebo | N/A |
| 5 | 150 | Licensed QIV | Placebo | N/A |
| 6^{a} | 150 | BNT162b2 | Placebo | RSVpreF |
| 7^{a} | 150 | BNT162b2 | QIV (Fluzone HD) | RSVpreF |

^{a}Separate administeration sites on right arm by 1 inch.

About 1 month after vaccination (28-35 days), blood samples were collected for neutralization titer analysis. About 6 months after vaccination, subjects were contacted by phone for follow-up questions.

In the present study, a bivalent SARS-CoV-2 RNA vaccine was administered (corresponding to BNT162b2, and delivering a full-length, prefusion-stabilized S protein of a Wuhan strain and an Omicron BA.4/5 variant). A person of skill in the art will understand, however, that, in some embodiments, alternative valencies may be administered (e.g., a monovalent, trivalent, or tetravalent SARS-CoV-2 vaccine). In some embodiments, the SARS-CoV-2 vaccine may deliver an S protein of one or more strains or variants that are prevalent in a relevant jurisdiction and/or which have been predicted by health authorities to be variants of concern.

Each vaccine, whether administered with placebo or in combination with other vaccines, was administered at the dose approved for the relevant population (patients 65 years and older). In the present study, BNT162b2 was administered at a dose comprising 30 μg of RNA (in 0.3 mL), RSVpreF was administered at a dose comprising 120 μg of RSV F antigen (in 0.5 mL) and Fluzone HD was administered at a dose comprising 180 μg of total HA protein (administered in 0.5 mL).

[RSVpreF+BNT162b2] indicates an admixture of the RSVpreF and BNT162b2 vaccines. In the present Example, the two vaccines were admixed immediately before administering to a subject (e.g., using a procedure as depicted in FIG. 3), so that the two vaccines were administered in a single shot. A person of skill in the art will understand, that (i) vaccines to be administered can be mixed prior to the clinic (e.g., during manufacturing), and (ii) if the vaccines are to be mixed immediately prior to administration, any suitable method known in the art may be used to admix the vaccines.

### Outcome Measures

### Primary Outcome Measures:

Percentage of participants reporting local reactions (for up to 7 days following vaccination)
Pain at the injection site, redness, and swelling
Percentage of participants reporting systemic events (for up to 7 days following vaccination)
Fever, fatigue, headache, vomiting, diarrhea, chills, new or worsened muscle pain, and new or worsened joint pain
Percentage of participants reporting adverse events (within 1 month of vaccination)
Percentage of participants reporting serious adverse events (within 6 months of vaccination)
Group 1 vs Group 4 noninferiority analysis: Geometric mean ratios (GMRs) of RSV A and RSV B neutralizing titers (NTs) (1 month after vaccination)
Group 1 vs Group 3 noninferiority analysis: Geometric mean ratios (GMRs) of SARS-CoV-2 Omicron BA.4/BA.5-strain neutralizing titers (NTs) (1 month after vaccination (Day 28))
Group 1 vs Group 3 noninferiority analysis: Geometric mean ratios (GMRs) of SARS-CoV-2 reference-strain neutralizing titers (NTs) (1 month after vaccination))
Group 1 vs Group 5 noninferiority analysis: Influenza strain-specific geometric mean ratios (GMRs) of hemagglutination inhibition (HAI) titers (1 month after vaccination (Day 28)).
Group 2 vs Group 4 noninferiority analysis: Geometric mean ratios (GMRs) of RSV A and RSV B neutralizing titers (NTs) (1 month after vaccination)
Group 2 vs Group 3 noninferiority analysis: Geometric mean ratios (GMRs) of SARS-CoV-2 Omicron BA.4/BA.5-strain neutralizing titers (NTs) (1 month after vaccination)
Group 2 vs Group 3 noninferiority analysis: Geometric mean ratios (GMRs) of SARS-CoV-2 reference-strain neutralizing titers NTs (1 month after vaccination)

### Secondary Outcome Measures:

Group 7 vs Group 4 noninferiority analysis: Geometric mean ratios (GMRs) of RSV A and RSV B neutralizing titers (NTs) (1 month after vaccination).
Group 7 vs Group 3 noninferiority analysis: Geometric mean ratios (GMRs) of SARS-CoV-2 Omicron BA.4/BA.5-strain neutralizing titers (NTs) (1 month after vaccination).
Group 7 vs Group 3 noninferiority analysis: Geometric mean ratios (GMRs) of SARS-CoV-2 reference-strain neutralizing titers (NTs) (1 month after vaccination)
Group 7 vs Group 5 noninferiority analysis: Influenza strain-specific geometric mean ratio (GMR) of hemagglutination inhibition (HAI) titers (1 month after vaccination)
Group 6 vs Group 4 noninferiority analysis: Geometric mean ratios (GMRs) of RSV A and RSV B neutralizing titers (NTs) (1 month after vaccination)
Group 6 vs Group 3 noninferiority analysis: Geometric mean ratios (GMRs) of SARS-CoV-2 Omicron BA.4/BA.5-strain neutralizing titers (NTs) (1 month after vaccination)
Group 6 vs Group 3 noninferiority analysis: Geometric mean ratios (GMRs) of SARS-CoV-2 reference-strain neutralizing titers (NTs) (1 month after vaccination)

### Results

Neutralization titers observed for each of Groups 2, 3, and 4 are shown in Table 57, on the following page. GMT ratios are shown in FIG. 4.

TABLE 57
Comparison of GMTs for [RSVpreF + BNT162b2] Coadministered With Placebo
vs Each Vaccine Administered Alone, 1 Month After Vaccination

| | | | | | | Comparison | | | |
|---|---|---|---|---|---|---|---|---|---|
| | | NT50 | | NT50 | NT50 | NT50 | | Comparison | Comparison |
| | | (Omicron | | (RSV A) | (RSV B) | (Omicron | | NT50 | NT50 |
| | | BA.4/5) | NT50 (Wuhan) | GMT^{b} | GMT^{b} | BA.4/5) | Comparison | (RSV A) | (RSV B) |
| | | GMT^{b} | GMT^{b} | (97.5% | (97.5% | GMR^{c} | NT50 | GMR^{c} | GMR^{c} |
| Group | Intervention | (97.5% CI) | (97.5% CI^{b}) | CI^{b}) | CI^{b}) | (97.5% CI^{c}) | (Wuhan) | (97.5% CI^{c}) | (97.5% CI^{c}) |
| 2 | 30 μg of | 3069 (2397.5, | 10470 (8472.7, | 18304 | 17963 | 0.83 (0.585, | 0.84 | 0.99 (0.764, | 1.08 (0.825, |
| | [BNT162b2 + | 3927.4) | 12938.1) | (15069.7, | (14766.4, | 1.173) | (0.625, | 1.281) | 1.407) |
| | RSVpreF] + | N^{a} = 146 | N^{a} = 147 | 22233.2) | 21851.0) | | 1.117) | | |
| | Placebo | | | N^{a} = 147 | N^{a} = 147 | | | | |
| 3 | 30 μg of | 3705 (2889.7, | 12,530 | N/A | N/A | N/A | N/A | N/A | N/A |
| | BNT162b2 + | 4749.1) | (10253.1, | | | | | | |
| | Placebo | N^{a} = 144 | 15312.3) | | | | | | |
| | | | N^{a} = 144 | | | | | | |
| 4 | RSVpreF + | N/A | N/A | 18498 | 16677 | N/A | N/A | N/A | N/A |
| | Placebo | | | (155570.0, | (13884, | | | | |
| | | | | 21975.9) | 20031.4) | | | | |
| | | | | N^{a} = 144 | N^{a} = 144 | | | | |

Abbreviations:
GMR = geometirc mean ratio;
GMT = geometric mean titer;
LLOQ = lower limit of quanitation;
NT50 = 50% neutralization titter;
[RSVpreF + BNT162b2] denotes admixture of RSVpreF and bivalent BNT162b2 (Wuhan + Omi BA.4/5) vaccines
LLOQ values were 242 for RSV A NT50, 99 for RSV B NT50, 71 for SARS-CoV-2 BA.4/5 NT50, and 87 for SARS-CoV-2 Wuhan NT50. Assay results below the LLOQ were set to 0.5 LLOQ.
^{a}N = number of participats with valid and determinate assay results for the specified assay in evaluable immunogenicity population
^{b}GMTs and the corresponding 2-sided Cis were calculated by exponentiating the mean logarithm of the titers and the corresponding Cis (based on the Student t distribution).
^{c}GMRs and 2-sided Cis were caculated by exponentiating the mean difference of the logarithms of the titers and the corresponding Cis (based on the Student t distribution).

As shown in Table 57 and FIG. 4, SARS-CoV-2 and RSV neutralization titers were found to be non-inferior for the [RSVpreF+BNT162b2] admixture as compared to the RSVpreF and BNT162b2 vaccines administered separately (based on a 2-fold margin). Similar results were seen for the admixed vaccine whether given with the concomitant QIV or administered alone (data not shown for subjects given a concomitant QIV). Similar results were also seen when the two admixed vaccine groups (Groups 1 and 2) were combined and analyzed, with narrower confidence intervals. For the 2 RSV antigens, the lower bounds remained greater than 0.667 (equivalent to a 1.5-fold margin). For the 2 SARS-CoV-2 antigens, the lower bounds were slightly below 0.667 (0.643 for Omicron BA.4/5 and 0.659 for the Wuhan strain).

From these results, it was concluded that non-inferiority of neutralization titers based on a 2-fold margin was met for both SARS-CoV-2 antigens and both RSV antigens for the admixed vaccine given with placebo, compared to the individual vaccines administered alone. For the 2 RSV antigens, non-inferiority of neutralization titers was also met based on a 1.5-fold margin. Similar results were seen for the admixed vaccine administered with concomitant QIV.

1. A vessel comprising a recently admixed combination comprising:
(a) a SARS-CoV-2 vaccine; and
(b) an influenza vaccine; wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the influenza vaccine: (i) is a nanoparticle (e.g., LNP) formulated RNA vaccine, or (ii) comprises one or more antigenic polypeptides (e.g., an HA protein) of one or more influenza virus strains.

2. A vessel comprising a recently admixed combination comprising:
(a) a SARS-CoV-2 vaccine; and
(b) an RSV vaccine;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains.

3. A vessel comprising a recently admixed combination comprising:
(a) a SARS-CoV-2 vaccine;
(b) an RSV vaccine;
(c) an influenza vaccine;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains; and
wherein the influenza vaccine: (i) is a nanoparticle (e.g., LNP) formulated RNA vaccine, or (ii) comprises one or more antigenic polypeptides (e.g., an HA protein) of one or more influenza virus strains.

4. The vessel of any one of claims 1-3, wherein the SARS-CoV-2 vaccine is a monovalent or bivalent vaccine.

5. The vessel of any one of claim 1, 3, or 4, wherein the influenza vaccine is a quadrivalent vaccine.

6. The vessel of any one of claim 1, 3, 4, or 5, wherein the influenza vaccine is an inactivated influenza virus, a recombinant influenza vaccine, a live attenuated influenza vaccine, a non-adjuvanted influenza vaccine, an adjuvanted influenza vaccine, or a subunit or split vaccine.

7. The vessel of any one of claims 2-6, wherein the RSV vaccine comprises a prefusion-stabilized F protein or an immunogenic fragment thereof of one or more RSV strains.

8. The vessel of any one of claims 1-7, wherein the vessel is a syringe or a vial.

9. A method of simultaneously vaccinating a human subject against each of SARS-CoV-2 and influenza, the method comprising:
simultaneously administering a SARS-CoV-2 vaccine composition and an influenza vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the influenza vaccine: (i) is a nanoparticle (e.g., LNP) formulated RNA vaccine, or (ii) comprises one or more antigenic polypeptides (e.g., an HA protein) of one or more influenza virus strains.

10. A method of simultaneously vaccinating a human subject against each of SARS-CoV-2 and RSV, the method comprising:
simultaneously administering a SARS-CoV-2 vaccine composition and an RSV vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs)); and
wherein the RSV vaccine comprises one or more antigenic polypeptides (e.g., an F protein or an immunogenic fragment thereof) associated with one or more RSV strains.

11. A method of simultaneously vaccinating a human subject against each of SARS-CoV-2, influenza, and RSV, the method comprising:
simultaneously administering a SARS-CoV-2 vaccine composition, an influenza vaccine composition, and an RSV vaccine composition to the same site;
wherein the SARS-CoV-2 vaccine comprises one or more RNAs that encode an immunogenic portion of a SARS-CoV-2 Spike (S) protein and which are formulated in nanoparticles (e.g., lipid nanoparticles (LNPs));
wherein the influenza vaccine: (i) is a nanoparticle (e.g., LNP) formulated RNA vaccine, or (ii) comprises one or more antigenic polypeptides (e.g., an HA protein) of one or more influenza virus strains; and
wherein the RSV vaccine comprises one or more antigenic polypeptides (e.g., an F protein or an immunogenic fragment thereof) associated with one or more RSV strains.

12. The method of claim 9, wherein the step of administering comprises injecting a composition through a needle or port; and
wherein the injected composition includes both the SARS-CoV-2 vaccine composition and the influenza vaccine composition; and
wherein the SARS-CoV-2 vaccine composition and the influenza vaccine composition are optionally administered using a syringe (e.g., a dual chamber syringe).

13. The method of claim 10, wherein the step of administering comprises injecting a composition through a needle or port; and
wherein the injected composition includes both the SARS-CoV-2 vaccine composition and the RSV vaccine composition; and
wherein the SARS-CoV-2 vaccine composition and the RSV vaccine composition are optionally administered using a syringe (e.g., a dual chamber syringe).

14. The method of claim 11, wherein the step of administering comprises injecting a composition through a needle or port;
wherein the injected composition includes each of the SARS-CoV-2 vaccine composition, the influenza vaccine composition, and the RSV vaccine composition; and
wherein the SARS-CoV-2 vaccine composition, the RSV vaccine composition, and the influenza vaccine composition are optionally administered using a syringe (e.g., a dual chamber syringe).

15. The method of claim 9 or 12, further comprising a step, prior to the step of administering, of admixing the SARS-CoV-2 vaccine composition and the influenza vaccine composition.

16. The method of claim 10 or 13, further comprising a step, prior to the step of administering, of admixing the SARS-CoV-2 vaccine composition and the RSV vaccine composition.

17. The method of claim 11 or 14, further comprising a step, prior to the step of administering, of admixing the SARS-CoV-2 vaccine composition, the influenza vaccine composition, and the RSV vaccine composition.

18. The method of any one of claims 15-17, wherein the step of admixing is performed within a period of time of the step of administering, which period of time is not more than 2 hours (e.g., not more than 1 hour, 30 minutes, 20 minutes, 15 minutes, 10 minutes, or 5 minutes).

19. The vessel of any one of claims 1-8 or the method of any one of claims 9-18, wherein the SARS-CoV-2 vaccine composition comprises two or more RNAs, each encoding an S protein of a different SARS-CoV-2 strain or variant, and wherein the two or more RNAs are encapsulated in separate populations of LNPs.

20. The vessel of any one of claims 1 and 3-8 or the method of any one of claims 9 and 11-18, or the vessel or method of claim 19, wherein the influenza vaccine comprises two or more RNAs (e.g., four RNAs), each encoding an antigenic polypeptide (e.g., HA protein) of a different influenza strain, and wherein the two or more RNAs are encapsulated in separate populations of LNPs.

21. The vessel of any one of claims 1-8, or the method of any one of claims 9-18, or the vessel or method of claim 19 or 20, wherein the SARS-CoV-2 vaccine comprises:
(a) (i) an RNA comprising a nucleotide sequence that includes modified uridines and encodes a first SARS-CoV-2 Spike (S) polypeptide from a SARS-CoV-2 strain, wherein the RNA encodes a polypeptide comprising an amino acid sequence that is at least 85% identical to SEQ ID NO: 7, and/or comprises a nucleotide sequence that is at least 85% identical to SEQ ID NO: 20 and/or a nucleotide sequence that is at least 85% identical to SEQ ID NO: 9, and (ii) an RNA comprising a nucleotide sequence that includes modified uridines and encodes a S polypeptide from an Omicron BA.4/5 SARS-CoV-2 variant, wherein the RNA comprises a nucleotide sequence that encodes a polypeptide comprising a sequence that is at least 85% identical to SEQ ID NO: 69 and/or wherein the RNA comprises a nucleotide sequence that is at least 85% identical to SEQ ID NO: 72 and/or a nucleotide sequence that is at least 85% identical to SEQ ID NO: 70; or
(b) an RNA comprising a nucleotide sequence that includes modified uridines and encodes a SARS-CoV-2 Spike (S) polypeptide, wherein the RNA comprises a nucleotide sequence that encodes a polypeptide comprising a sequence that is at least 85% identical to SEQ ID NO: 129 and/or wherein the RNA comprises a nucleotide sequence that is at least 85% identical to SEQ ID NO: 132 and/or a nucleotide sequence that is at least 85% identical to SEQ ID NO: 130.

22. The vessel of any one of claims 1-8, or the method of any one of claims 9-18, or the vessel or method of claim 19 or 20, wherein the influenza vaccine comprises:
(a) (i) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94 and/or a nucleotide sequence that is at least 85% identical to SEQ ID NO: 92; (ii) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99 and/or a nucleotide sequence that is at least 85% identical to SEQ ID NO: 97; (iii) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104 and/or a nucleotide sequence that is at least 85% identical to SEQ ID NO: 102; and (iv) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109 and/or a nucleotide sequence that is at least 85% identical to SEQ ID NO: 107; or
(b) (i) an RNA comprising a nucleotide sequence that includes modified uridines and encodes an influenza hemagglutinin antigen from an influenza A H1N1 strain, wherein the nucleotide sequence is at least 85% identical to SEQ ID NO: 92 and/or at least 85% identical to SEQ ID NO: 94; (ii) an RNA comprising a nucleotide sequence that includes modified uridines and encodes an influenza hemagglutinin antigen from an influenza A H3N2 strain, wherein the nucleotide sequence is at least 85% identical to SEQ ID NO: 82 and/or at least 85% identical to SEQ ID NO: 84; (iii) an RNA comprising a nucleotide sequence that includes modified uridines and encodes an influenza hemagglutinin antigen from an influenza B Victoria strain, wherein the nucleotide sequence is at least 85% identical to SEQ ID NO: 87 and/or that is at least 85% identical to SEQ ID NO: 89; and (iv) an RNA comprising a nucleotide sequence that includes modified uridines and encodes an influenza hemagglutinin antigen from an influenza B Yamagata strain, wherein the nucleotide sequence is at least 85% identical to SEQ ID NO: 107 and/or that is at least 85% identical to SEQ ID NO: 109.

23. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 9;
(ii) an RNA comprising a second nucleotide sequence that includes modified uridines and encodes a second SARS-CoV-2 Spike (S) polypeptide from a variant of the SARS-CoV-2 strain, wherein the second nucleotide sequence is at least 85% identical to SEQ ID NO: 70;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 92;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 97;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 102; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 107.

24. A composition comprising:
(i) an RNA comprising a nucleotide sequence that includes modified uridines and encodes a first SARS-CoV-2 Spike (S) polypeptide from a SARS-CoV-2 strain, wherein the RNA comprises a nucleotide sequence that is at least 85% identical to SEQ ID NO: 20;
(ii) an RNA comprising a nucleotide sequence that includes modified uridines and encodes a second SARS-CoV-2 Spike (S) polypeptide from a variant of the SARS-CoV-2 strain, wherein the RNA comprises a nucleotide sequence that is at least 85% identical to SEQ ID NO: 72;
(iii) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94;
(iv) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99;
(v) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104; and
(vi) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109.

25. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 9;
(ii) an RNA comprising a second nucleotide sequence that includes modified uridines and encodes a second SARS-CoV-2 Spike (S) polypeptide from a variant of the SARS-CoV-2 strain, wherein the second nucleotide sequence is at least 85% identical to SEQ ID NO: 70;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 92;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 82;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 87; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 107.

26. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 20;
(ii) an RNA comprising a second nucleotide sequence that includes modified uridines and encodes a second SARS-CoV-2 Spike (S) polypeptide from a SARS-CoV-2 strain, wherein the first nucleotide sequence is at least 85% identical to SEQ ID NO: 72;
(iii) an RNA comprising a third nucleotide sequence that includes modified uridines and encodes an influenza hemagglutinin antigen from an influenza A H1N1 strain, wherein the third nucleotide sequence is at least 85% identical to SEQ ID NO: 94;
(iv) an RNA comprising a fourth nucleotide sequence that includes modified uridines and encodes an influenza hemagglutinin antigen from an influenza A H3N2 strain, wherein the fourth nucleotide sequence is at least 85% identical to SEQ ID NO: 84;
(v) an RNA comprising a fifth nucleotide sequence that includes modified uridines and encodes an influenza hemagglutinin antigen from an influenza B Victoria strain, wherein the fifth nucleotide sequence is at least 85% identical to SEQ ID NO: 89; and
(vi) an RNA comprising a sixth nucleotide sequence that includes modified uridines and encodes an influenza hemagglutinin antigen from an influenza B Yamagata strain, wherein the sixth nucleotide sequence is at least 85% identical to SEQ ID NO: 109.

27. The composition of any one of claims 23-26, wherein:
(a) the mass ratio of RNAs (i)-(ii) to RNAs (iii)-(vi) is 1:5 to 5:1, 1:4 to 4:1, 1:3 to 3:1, 1:2 to 2:1;
(b) the mass ratio of RNAS (iii)-(iv) to RNAs (v)-(vi) is 1:1 to 1:5; and/or
(c) the mass ratio of RNA (i) to RNA (ii) is 1:1.

28. The composition of any one of claims 23-27, wherein RNAs (iii), (iv), (v), and (vi) are present in a mass ratio of 1:1:1:1 or 1:1:5:5.

29. The composition of any one of claim 23-28, wherein the combined mass of RNAs (i)-(vi) is about 30 μg to about 100 ug.

30. The composition of any one of claims 23-29, wherein:
the combined mass of RNAs (i)-(ii) is about 3 μg to about 60 μg (e.g., about 3 μg, about 10 μg, about 30 μg, or about 60 μg); and/or
wherein the combined mass of RNAs (iii)-(vi) is about 30 μg to about 60 μg (e.g., about 30 μg or about 60 μg).

31. The composition of any one of claims 23-29, wherein:
(a) RNA (i) and (ii) are each present in an amount of about 15 μg, and RNAs (iii)-(vi) are each present in an amount of about 7.5 μg;
(b) RNA (i) and (ii) are each present in an amount of about 30 μg, and RNAs (iii)-(vi) are each present in an amount of about 7.5 μg;
(c) RNA (i) and (ii) are each present in an amount of about 15 μg, and RNAs (iii)-(vi) are each present in an amount of about 11.25 μg;
(d) RNA (i) and (ii) are each present in an amount of about 15 μg, RNAs (iii) and (iv) are each present in an amount of about 5 μg, and RNAs (v) and (vi) are each present in an amount of about 25 μg;
(e) RNA (i) and (ii) are each present in an amount of about 15 μg, RNAs (iii) and (iv) are each present in an amount of about 2.5 μg, and RNAs (v) and (vi) are each present in an amount of about 12.5 μg;
(f) RNA (i) and (ii) are each present in an amount of about 30 μg, RNAs (iii) and (iv) are each present in an amount of about 2.5 μg, and RNAs (v) and (vi) are each present in an amount of about 12.5 μg; or
(g) RNA (i)-(vi) are each present in an amount of about 15 μg.

32. A composition comprising:
(i) an RNA comprising a first nucleotide sequence that includes modified uridines and encodes a SARS-CoV-2 Spike (S) polypeptide, wherein the first nucleotide sequence is at least 85% identical to SEQ ID NO: 129;
(ii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 92;
(iii) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 99;
(iv) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 102; and
(v) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 107.

33. A composition comprising:
(i) an RNA comprising a nucleotide sequence that includes modified uridines and encodes a SARS-CoV-2 Spike (S) polypeptide, wherein the RNA comprises a nucleotide sequence that is at least 85% identical to SEQ ID NO: 132;
(ii) an RNA comprising a nucleotide sequence that includes modified uridines and encodes an influenza hemagglutinin antigen from an influenza A H1N1 strain, wherein the RNA comprises a nucleotide sequence that is at least 85% identical to SEQ ID NO: 94;
(iii) an RNA comprising a nucleotide sequence that includes modified uridines and encodes an influenza hemagglutinin antigen from an influenza A H3N2 strain, wherein the RNA comprises a nucleotide sequence that is at least 85% identical to SEQ ID NO: 99;
(iv) an RNA comprising a nucleotide sequence that includes modified uridines and encodes an influenza hemagglutinin antigen from an influenza B Victoria strain, wherein the RNA comprises a nucleotide sequence that is at least 85% identical to SEQ ID NO: 104; and
(v) an RNA comprising a nucleotide sequence that includes modified uridines and encodes an influenza hemagglutinin antigen from an influenza B Yamagata strain, wherein the RNA comprises a nucleotide sequence that is at least 85% identical to SEQ ID NO: 109.

34. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 130;
(ii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 92;
(iii) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 82;
(iv) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 87; and
(v) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 107.

35. A composition comprising:
(i) an RNA comprising a first nucleotide sequence that includes modified uridines and encodes a first SARS-CoV-2 Spike (S) polypeptide from a SARS-CoV-2 strain, wherein the first nucleotide sequence is at least 85% identical to SEQ ID NO: 132;
(ii) an RNA comprising a second nucleotide sequence that includes modified uridines and encodes an influenza hemagglutinin antigen from an influenza A H1N1 strain, wherein the second nucleotide sequence is at least 85% identical to SEQ ID NO: 94;
(iii) an RNA comprising a third nucleotide sequence that includes modified uridines and encodes an influenza hemagglutinin antigen from an influenza A H3N2 strain, wherein the third nucleotide sequence is at least 85% identical to SEQ ID NO: 84;
(iv) an RNA comprising a fourth nucleotide sequence that includes modified uridines and encodes an influenza hemagglutinin antigen from an influenza B Victoria strain, wherein the fourth nucleotide sequence is at least 85% identical to SEQ ID NO: 89; and
(v) an RNA comprising a fifth nucleotide sequence that includes modified uridines and encodes an influenza hemagglutinin antigen from an influenza B Yamagata strain, wherein the fifth nucleotide sequence is at least 85% identical to SEQ ID NO: 109.

36. The composition of any one of claims 32-35, wherein:
RNA (i) and RNAS (ii)-(v) are present in a mass ratio of 1:5 to 5:1, 1:4 to 4:1, 1:3 to 3:1, or 1:2 to 2:1;
RNAs (ii) and (iii) and RNAs (iv) and (v) are present in a mass ratio of 1:1 to 1:5;
wherein RNAs (ii), (iii), (iv), and (v) are present in a mass ratio of 1:1:1:1 or 1:1:5:5.

37. The composition of any one of claim 24-36, wherein the combined mass of RNAS (i)-(v) is 30 ug to 100 ug.

38. The composition of any one of claims 32-37, wherein the mass of RNA (i) is about 3 μg to about 60 μg (e.g., about 3 μg, about 10 μg, about 30 μg, or about 60 μg), and/or wherein the combined mass of RNAS (ii)-(v) is about 30 μg to about 60 μg (e.g., about 30 μg or about 60 μg).

39. The composition of any one of claims 32-38, wherein:
(a) RNA (i) is present in an amount of about 30 μg, and RNAS (ii)-(v) are each present in an amount of about 7.5 μg;
(b) RNA (i) is present in an amount of about 60 μg, and RNAs (ii)-(v) are each present in an amount of about 7.5 μg;
(c) RNA (i) is present in an amount of about 30 μg, and RNAs (ii)-(v) are each present in an amount of about 11.25 μg;
(d) RNA (i) is present in an amount of about 30 μg, RNAs (ii) and (iii) are each present in an amount of about 5 μg, and RNAs (iv) and (v) are each present in an amount of about 25 μg;
(e) RNA (i) is present in an amount of about 30 μg, RNAs (ii) and (iii) are each present in an amount of about 2.5 μg, and RNAs (iv) and (v) are each present in an amount of about 12.5 μg;
(f) RNA (i) is present in an amount of about 30 μg, RNAs (ii) and (iii) are each present in an amount of about 2.5 μg, and RNAs (iv) and (v) are each present in an amount of about 12.5 μg; or
(g) RNA (i) is present in an amount of about 30 μg, and RNAS (ii)-(v) are each present in an amount of about 15 μg.

40. The composition of any one of claims 23-39, wherein the influenza A H1N1 strain is Influenza A/Wisconsin/588/2019 and wherein the influenza B Yamagata strain is Influenza B/PHUKET/3073/2013.

41. The composition of any one of claims 23, 24, 27-31, 33, 34, and 37-40, wherein the influenza A H3N2 strain is Influenza A/Cambodia/e0826360/2020, and wherein the influenza B Victoria strain is Influenza B/Washington/02/2019.

42. The composition of any one of claims 25-31 and 34-39, wherein the influenza A H3N2 strain is Influenza A/Darwin/6/2021

43. The composition of any one of claims 25-31 and 34-40, wherein the influenza A H3N2 strain is Influenza A/Darwin/6/2021 and/or wherein the influenza B Victoria strain is Influenza B/Austria/1359417/2021.

44. The composition of any one of claims 23-31, wherein the first SARS-CoV-2 Spike (S) polypeptide is from a Wuhan strain and wherein the second SARS-CoV-2 S polypeptide is from an Omicron BA.4/5 variant.

45. The composition of any one of claims 32-43, wherein the SARS-CoV-2 Spike (S) polypeptide is from an XBB.1.5 variant.

46. The composition of any one of claims 23-45, wherein each of the RNAs in the composition comprises the same non-coding elements that include the same 5′ cap, cap proximal sequence, 5′ UTR sequence, 3′ UTR sequence, and polyA sequence.

47. A composition comprising:
(i) a coronavirus RNA vaccine comprising one or more RNAs, each comprising a nucleotide sequence that encodes a SARS-CoV-2 antigen; and
(ii) an influenza RNA vaccine comprising one or more RNAs, each comprising one or more nucleotide sequences that encode an influenza antigen, wherein the influenza RNA vaccine encodes at least four influenza antigens, and wherein each influenza antigen is from a distinct influenza virus strain that is predicted to circulate during a flu season of a particular hemisphere;
wherein each RNA in the composition comprises the same non-coding elements, including the same 5′ cap, cap proximal sequence, 5′ UTR sequence, 3′ UTR sequence, and polyA sequence.

48. The composition of claim 47, wherein each of the one or more RNAs in the coronavirus RNA vaccine and each of the one or more RNAs in the influenza RNA vaccine include one or more modified uridines.

49. The composition of claim 47 or 48, wherein the at least four influenza antigens each are or comprise a hemagglutinin antigen from a distinct influenza virus strain predicted to circulate during a flu season of a particular hemisphere.

50. The composition of claim 49, wherein the distinct influenza virus is predicted to circulate during a flu season based on human serology data from the Northern or Southern hemisphere.

51. The composition of any one of claims 47-50, wherein the at least four influenza antigens are each encoded by a separate RNA.

52. The composition of any one of claims 47-51, wherein the coronavirus RNA vaccine encodes at least two SARS-CoV-2 antigens, each from a distinct SARS-CoV-2 strain or variant.

53. The composition of claim 52, wherein the at least two SARS-CoV-2 antigens are or comprise a SARS-CoV-2 Spike (S) polypeptide from a SARS-CoV-2 strain and a SARS-CoV-2 S polypeptide from a variant of the SARS-CoV-2 strain.

54. The composition of claim 52 or 53, wherein the at least two SARS-CoV-2 antigens are each encoded by a separate RNA.

55. The composition of any one of claims 47-54, wherein the RNAs in the coronavirus vaccine and the RNAs in the influenza vaccine are present in a mass ratio of 1:5 to 5:1, 1:4 to 4:1, 1:3 to 3:1, or 1:2 to 2:1.

56. The composition of any one of claims 47-55, wherein the at least four influenza antigens comprise at least two hemagglutinin antigens from influenza A viruses and at least two hemagglutinin antigens from influenza B viruses.

57. The composition of claim 56, wherein the RNAs that encode hemagglutinin antigens from influenza A viruses and the RNAs that encode hemagglutinin antigens from influenza B viruses are present in a mass ratio of 1:1 to 1:5 (e.g., 1:1 or 1:5).

58. The composition of any one of claims 54-57, wherein the at least two RNAs in the coronavirus vaccine are in a mass ratio of 1:1.

59. The composition of any one of claims 51-58, wherein the at least four RNAs in the influenza vaccine are present in a mass ratio of 1:1:1:1.

60. The composition of any one of claims 47-59, wherein the total amount of RNA in the composition is about 30 ug to about 100 ug (e.g., about 30 ug, about 45 ug, about 60 ug, about 75 ug, or about 90 ug).

61. A composition comprising:
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent, wherein the second infectious agent is different from the first infectious agent;
wherein each of the first and second RNAs in the composition comprises the same non-coding elements, including the same 5′ cap, cap proximal sequence, 5′ UTR sequence, 3′ UTR sequence, and polyA sequence, and
wherein at least one of the same non-coding elements is or comprises:
(i) a 5′-UTR sequence that is or comprises a modified human alpha-globin 5′-UTR;
(ii) a 3′-UTR sequence that is or comprises a first sequence from the amino terminal enhancer of split (AES) messenger RNA and a second sequence from the mitochondrial encoded 12S ribosomal RNA;
(iii) a polyA sequence comprising at least 100 A nucleotides, wherein the first RNA and the second RNA each do not comprise a stretch of at least 30 contiguous C nucleotides between the 3′ UTR and the polyA sequence;
(iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
(v) a 5′ cap comprising a Cap1 structure and a cap proximal sequence comprising positions +1, +2, +3, +4, and +5 of the RNA; wherein:
(a) the Cap1 structure comprises m7(3′OMeG)(5′)ppp(5′)(2′OMeA₁)pG₂, wherein A₁ is position +1 of the RNA, and G₂ is position +2 of the RNA; and
(b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

62. A composition comprising:
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent that is different from the first infectious agent;
wherein each of the first and second RNAs in the composition comprises the same non-coding elements including the same 5′ cap, cap proximal sequence, 5′ UTR sequence, 3′ UTR sequence, and polyA sequence, and
wherein each of the first and second RNAs is characterized in that:
(i) an immune response induced by the RNA in the composition has a level that is at least 80% of a level of an immune response induced by the same RNA when it is administered alone; and/or
(ii) an immune response induced by the RNA in the composition has a level that is at least 80% of a level of an immune response induced by the same RNA when it is administered separately from the other RNAs at a different location of a subject's body; and/or
(iii) an immune response induced by the RNA in the composition has a level that is at least 80% of a level of an immune response induced by a respective reference composition.

63. The composition of claim 62, wherein the respective reference composition is an inactivated virus vaccine.

64. The composition of claim 62 or 63, wherein the immune response induced by the one or more first RNA(s) and the one or more second RNA(s) are each at least 100% of a level of an immune response induced by the same RNA when the one or more first RNA(s) and the one or more second RNA(s) are administered separately.

65. The composition of any one of claims 62-64, wherein the immune response induced by the one or more first RNA(s) and the one or more second RNA(s) are each greater than an immune response induced by the same RNAs administered separately.

66. The composition of any one of claims 62-64, wherein the one or more first RNA(s) and the one or more second RNA(s) are each present at a dose that is lower than that of the same RNAs administered separately, wherein the immune response induced by the lower dose of the one or more first RNA(s) and the one or more second RNA(s) are each substantially comparable to or greater than the immune response induced by a greater dose of the same RNAs administered separately.

67. A composition comprising:
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent that is different from the first infectious agent;
wherein each of the first and second RNAs in the composition comprises the same non-coding elements, including the same 5′ cap, cap proximal sequence, 5′ UTR sequence, 3′ UTR sequence, and polyA sequence,
wherein each of the first and second RNAs is encapsulated, separately or together, in nanoparticles (e.g., each of the first RNAs is encapsulated in a first population of nanoparticles and each of the second RNAs is encapsulated in a second population of nanoparticles; or each of the first RNAs and each of the second RNAs is encapsulated in the same population of nanoparticles); and
wherein the composition is characterized in that:
(i) RNA content of the composition is at least 95% that of the initial RNA content after storing for 24 hours;
(ii) RNA encapsulation remains at least 95% that of the initial RNA encapsulation after storing for 24 hours;
(iii) the nanoparticles encapsulating the first and second RNAs have maintained substantially the same size after storing for 24 hours;
(iv) the nanoparticles encapsulating the first and second RNAs have maintained a polydispersity of no more than 0.3 after 24 hours; and/or
(v) the mass ratio of the first RNA and the second RNA remains substantially the same after storing for 24 hours.

68. The composition of claim 67, wherein the nanoparticles comprise lipid nanoparticles, polyplexes (PLX), lipidated polyplexes (LPLX), liposomes, or polysaccharide nanoparticles.

69. The composition of claim 68, wherein the nanoparticles comprise lipid nanoparticles.

70. The composition of claim 69, wherein the lipid nanoparticles comprise: a cationically ionizable lipid, one or more neutral lipids, and a polymer-conjugated lipid.

71. The composition of claim 70, wherein the polymer-conjugated lipid comprises a PEG-conjugated lipid.

72. The composition of any one of claims 67-71, wherein the nanoparticles have an average diameter of about 50-150 nm.

73. The composition of any one of claims 67-72, wherein, for each of (i)-(v), the first 12 hours of storing is at 30° C. and the remaining 12 hours of storing is at 2-8° C.

74. The composition of any one of claims 67-73, wherein the one or more first RNAs comprise at least two first RNAs, each comprising a nucleotide sequence that encodes one or more antigenic polypeptides associated with a different strain or variant of the first infectious agent.

75. The composition of any one of claims 67-74, wherein the one or more second RNAs comprise at least two second RNAs, each comprising a nucleotide sequence that encodes one or more antigenic polypeptides associated with a different strain or variant of the second infectious agent.

76. The composition of any one of claims 67-75, wherein the one or more second RNAs comprise at least three second RNAs, each comprising a nucleotide sequence that encodes one or more antigenic polypeptides associated with a different strain or variant of the second infectious agent.

77. The composition of any one of claims 67-76, wherein the one or more second RNAs comprise at least four second RNAs, each comprising a nucleotide sequence that encodes one or more antigenic polypeptides associated with a different variant or strain of the second infectious agent.

78. A composition comprising:
a plurality of first RNAs each comprising a nucleotide sequence that encodes one or more antigenic polypeptides associated with a first infectious agent of a different strain and/or variant thereof;
one or more second RNAs each comprising a nucleotide sequence that encodes one or more antigenic polypeptides associated with a second infectious agent that is different from the first infectious agent;
and wherein each of the first and second RNAs is formulated, either separately or together, in the same nanoparticle formulation;
wherein (i) the first RNAs and the second RNAs are present in a mass ratio of 1:2 to 2:1 and/or (ii) the first RNAs and second RNAs are present in the total amount of about 10 ug to about 100 ug per dose; and
one or more first RNAs, each comprising a nucleotide sequence that encodes one or more antigenic polypeptides associated with a first infectious agent;
one or more second RNAs, each comprising a nucleotide sequence that encodes one or more antigenic polypeptides associated with a second infectious agent, wherein the second infectious agent is different from the first infectious agent;
wherein each of the first and second RNAs in the composition comprises the same non-coding elements, including the same 5′ cap, cap proximal sequence, 5′ UTR sequence, 3′ UTR sequence, and polyA sequence, and
wherein at least one of the same non-coding elements is or comprises:
(i) a 5′-UTR sequence that is or comprises a modified human alpha-globin 5′-UTR;
(ii) a 3′-UTR sequence that is or comprises a first sequence from the amino terminal enhancer of split (AES) messenger RNA and a second sequence from the mitochondrial encoded 12S ribosomal RNA;
(iii) a polyA sequence comprising at least 100 A nucleotides, wherein the first RNA and the second RNA each do not comprise a stretch of at least 30 contiguous C nucleotides between the 3′ UTR and the polyA sequence;
(iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
(v) a 5′ cap comprising a Cap1 structure and a cap proximal sequence comprising positions +1, +2, +3, +4, and +5 of the RNA; wherein:
(a) the Cap1 structure comprises m7(3′OMeG)(5′)ppp(5′)(2′OMeA₁)pG₂, wherein A₁ is position +1 of the RNA, and G₂ is position +2 of the RNA; and
(b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

79. The composition of any one of claims 61-78, wherein each of the first RNAs is co-formulated in the same nanoparticle formulation or wherein each of the first RNAs is formulated in separate nanoparticle formulations.

80. The composition of any one of claims 61-79, wherein each of the second RNAs is co-formulated in the same nanoparticle formulation or wherein each of the second RNAs is formulated in separate nanoparticle formulations.

81. The composition of any one of claims 61-80, wherein the first RNAs and the second RNAs are formulated in separate populations of nanoparticles.

82. The composition of any one of claims 61-81, wherein the first RNAs and the second RNAs are all co-formulated in the same nanoparticle formulation.

83. The composition of any one of claims 61-82, wherein the first infectious agent is or comprises a coronavirus.

84. The composition of claim 83, wherein the one or more first RNAs comprises (i) an RNA comprising a nucleotide sequence that encodes one or more antigenic polypeptides associated with a first coronavirus and (ii) an RNA comprising a nucleotide sequence that encodes one or more antigenic polypeptides associated with a second coronavirus.

85. The composition of any one of claims 61-84, wherein the one or more second RNAs comprise a plurality of second RNAs, each comprising a nucleotide sequence that encodes one or more antigenic polypeptides associated with a second infectious agent of a different strain and/or variant thereof.

86. The composition of any one of claims 61-85, wherein the one or more second RNAs comprise at least two second RNAs, each comprising a nucleotide sequence that encodes one or more antigenic polypeptides associated with a second infectious agent of a different strain and/or variant thereof.

87. The composition of any one of claims 61-86, wherein the one or more second RNAs comprise at least three second RNAs, each comprising a nucleotide sequence that encodes one or more antigenic polypeptides associated with a second infectious agent of a different strain and/or variant thereof.

88. The composition of any one of claims 61-87, wherein the one or more second RNAs comprise at least four second RNAs, each comprising a nucleotide sequence that encodes one or more antigenic polypeptides associated with a second infectious agent of a different strain and/or variant thereof.

89. The composition of any one of claims 61-88, wherein the second infectious agent is or comprises a bacterial infectious agent.

90. The composition of claim 89, wherein the bacterial infectious agent is Streptococcus pneumoniae.

91. The composition of any one of claims 61-88, wherein the second infectious agent is or comprises a viral infectious agent.

92. The composition of claim 91, wherein the second infectious agent is a viral infectious agent that induces an infectious respiratory disease.

93. The composition of claim 92, wherein the viral infectious agent is or comprises an influenza virus, a pneumoviridae virus, or a Paramyxoviridae virus.

94. The composition of claim 93, wherein the Pneumoviridae virus is a Respiratory syncytial virus (RSV).

95. The composition of claim 93, wherein the infectious respiratory disease is or comprises an influenza type A, type B, and/or type C virus.

96. The composition of claim 95, wherein the infectious respiratory disease is or comprises an influenza type A, and/or type B virus.

97. The composition of claim 96, wherein the one or more second RNAs comprise (i) at least one RNA comprising a nucleotide sequence that encodes one or more antigenic polypeptides associated with influenza type A virus and (ii) at least one RNA comprising a nucleotide sequence that encodes one or more antigenic polypeptides associated with influenza type B virus.

98. The composition of claim 96 or 97, wherein the one or more second RNAs comprise (i) at least two RNAs, each comprising a nucleotide sequence that encodes one or more antigenic polypeptides associated with a different strain of an influenza type A virus, and (ii) at least two RNAs, each comprising a nucleotide sequence that encodes one or more antigenic polypeptides associated with a different strain of an influenza type B virus.

99. The composition of any one of claims 95-98, wherein the antigenic polypeptide(s) associated with each influenza virus is/are each independently a Hemagglutinin (HA) polypeptide, a neuraminidase (NA) polypeptide, or combinations thereof, or immunogenic fragments thereof.

100. The composition of any one of claims 96-99, wherein the strain(s) of the influenza type A and influenza type B viruses have each been predicted to be or is a circulating strain in the coming flu season, for example, based on human serology data.

101. The composition of any one of claims 96-100, wherein the strain(s) of the influenza A virus are selected from an H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, H10N7, and an H10N8 virus.

102. The composition of claim 101, wherein the strain(s) of the influenza type A virus is selected from an H1N1, H3N2, H5N1, and an H5N8 virus.

103. The composition of any one of claims 98-102, wherein the one or more second RNAs comprise an RNA comprising a nucleotide sequence encoding an antigenic polypeptide associated with an H1N1 virus.

104. The composition of claim 103, where the H1N1 virus is A/Wisconsin/588/2019.

105. The composition of claim 104, wherein the antigenic polypeptide associated with A/Wisconsin/588/2019 is an HA polypeptide and comprises an amino acid sequence that is at least 85% identical to SEQ ID NO: 90.

106. The composition of claim 104 or 105, wherein the antigenic polypeptide associated with A/Wisconsin/588/2019 is an HA polypeptide and the RNA encoding the HA polypeptide comprises a nucleotide sequence that is at least 85% identical to SEQ ID NO: 92.

107. The composition of any one of claims 98-106, wherein the one or more second RNAs comprise an RNA comprising a nucleotide sequence encoding an antigenic polypeptide associated with an H3N2 virus.

108. The composition of claim 107, wherein the H3N2 virus is A/Cambodia/e0826360/2020.

109. The composition of claim 108, wherein the antigenic polypeptide associated with A/Cambodia/e0826360/2020 is an HA polypeptide and comprises an amino acid sequence that is at least 85% identical to SEQ ID NO: 95.

110. The composition of claim 108 or 109, wherein the antigenic polypeptide associated with A/Cambodia/e0826360/2020 is an HA polypeptide, and the RNA encoding the HA polypeptide comprises a nucleotide sequence that is at least 85% identical to SEQ ID NO: 92.

111. The composition of claim 107, wherein the H3N2 virus is A/Darwin/6/2021.

112. The composition of claim 111, wherein the antigenic polypeptide associated with A/Darwin/6/2021 is an HA polypeptide and comprises an amino acid sequence that is at least 85% identical to SEQ ID NO: 80.

113. The composition of claim 111 or 112, wherein the antigenic polypeptide associated with A/Darwin/6/2021 is an HA polypeptide and the RNA encoding the HA polypeptide comprises a nucleotide sequence that is at least 85% identical to SEQ ID NO: 82.

114. The composition of any one of claims 98-113, wherein the one or more second RNAs comprise an RNA comprising a nucleotide sequence encoding an antigenic polypeptide associated with a B/Yamagata or B/Victoria lineage virus.

115. The composition of claim 114, where the B/Victoria lineage influenza virus is B/Washington/02/2019.

116. The composition of claim 115, wherein the antigenic polypeptide associated with B/Washington/02/2019 is an HA polypeptide and comprises a sequence that is at least 85% identical to SEQ ID NO: 100.

117. The composition of claim 115 or 116, wherein the antigenic polypeptide associated with B/Washington/02/2019 is an HA polypeptide, and the RNA encoding the HA polypeptide comprises a nucleotide sequence that is at least 85% identical to SEQ ID NO: 102.

118. The composition of claim 114, where the B/Victoria lineage influenza virus is B/Austria/1359417/2021.

119. The composition of claim 118, wherein the antigenic polypeptide associated with B/Austria/1359417/2021 is an HA polypeptide and comprises a sequence that is at least 85% identical to SEQ ID NO: 85.

120. The composition of claim 118 or 119, wherein the antigenic polypeptide associated with B/Austria/1359417/2021 is an HA polypeptide and the RNA encoding the HA polypeptide comprises a nucleotide sequence that is at least 85% identical to SEQ ID NO: 87.

121. The composition of claim 114, where the B/Yamagata lineage influenza virus is B/Phuket/3073/2013.

122. The composition of claim 121, wherein the antigenic polypeptide associated with B/Phuket/3073/2013 is an HA polypeptide and comprises an amino acid sequence that is at least 85% identical to SEQ ID NO: 105.

123. The composition of claim 121 or 122, wherein the antigenic polypeptide associated with B/Phuket/3073/2013 is an HA polypeptide, and the RNA encoding the HA polypeptide comprises a nucleotide sequence that is at least 85% identical to SEQ ID NO: 107.

124. The composition of any one of claims 61-123, wherein the first infectious agent is a coronavirus.

125. The composition of claim 124, wherein the coronavirus is an alphacoronavirus, a betacoronavirus, a gammacoronavirus, or a deltacoronavirus.

126. The composition of claim 125, wherein the coronavirus is a betacoronavirus.

127. The composition of claim 126, wherein the betacoronavirus is a sarbecovirus, a merbecovirus, an embecorvius, a nobecovirus, or a hibecorvirus.

128. The composition of claim 127, wherein the sarbecovirus is SARS-CoV-1 or SARS-CoV-2.

129. The composition of claim 128, wherein the sarbecovirus is SARS-CoV-2.

130. The composition of claim 127, wherein the merbecovirus is MERS-COV.

131. The composition of claim 129, wherein the one or more first RNAs comprise an RNA comprising a nucleotide sequence that encodes one or more antigenic polypeptides associated with a SARS-CoV-2 variant that is prevalent or has been identified as a variant of concern in a relevant population at the time of administration.

132. The composition of 129, wherein the one or more first RNAs comprise an RNA comprising a nucleotide sequence that encodes one or more antigenic polypeptides associated with an Omicron SARS-CoV-2 variant (e.g., a BA.1, BA.2, BA.4/5, or XBB.1.5 variant).

133. The composition of claim 129, wherein the one or more first RNAs comprise (i) an RNA comprising a nucleotide sequence that encodes one or more antigenic polypeptides associated with a first SARS-CoV-2 strain, wherein the first SARS-CoV-2 strain is a SARS-CoV-2 ancestral strain (Wuhan strain) and (ii) an RNA comprising a nucleotide sequence that encodes one or more antigenic polypeptides associated with a second SARS-CoV-2 variant, wherein the second SARS-CoV-2 is a variant of the SARS-CoV-2 ancestral strain, and is prevalent or has been identified as a variant of concern in a relevant population at the time of administration.

134. The composition of claim 129, wherein the one or more first RNAs comprise (i) an RNA comprising a nucleotide sequence that encodes one or more antigenic polypeptides associated with a first SARS-CoV-2 variant and (ii) an RNA comprising a nucleotide sequence that encodes one or more antigenic polypeptides associated with a second SARS-CoV-2 variant, wherein the first and the second SARS-CoV-2 variant are each prevalent or have been identified as a variant of concern in a relevant population at the time of administration.

135. The composition of claim 133 or 134, wherein the second SARS-CoV-2 variant is an Omicron variant of SARS-CoV-2.

136. The composition of claim 135, wherein the Omicron variant of SARS-CoV-2 is or comprises an Omicron BA.1, BA.2, BA.4/5, or XBB.1.5 variant.

137. The composition of any one of claims 124-136, wherein the antigenic polypeptide(s) associated with the coronavirus is a Spike (S) polypeptide, or a immunogenic fragment or variant thereof.

138. The composition of claim 137, wherein the S polypeptide is a prefusion stabilized S polypeptide.

139. The composition of claim 138, wherein the prefusion stabilized S polypeptide comprises at least two proline substitutions.

140. The composition of claim 139, wherein the two proline substitutions comprises proline residues at positions corresponding to residues 986 and 987 of SEQ ID NO: 1.

141. The composition of any one of claims 138-140, wherein the prefusion stabilized S polypeptide comprises at least six proline substitutions.

142. The composition of claim 141, wherein four of the at least six proline substitutions comprises proline residues at positions corresponding to residues 817, 892, 899, and 942 of SEQ ID NO: 1.

143. The composition of 132, wherein the RNA encoding one or more antigenic polypeptides associated with an Omicron SARS-CoV-2 variant encodes an S protein associated with an XBB.1.5 strain and comprising an amino acid sequence that is at least 85% identical to SEQ ID NO: 129.

144. The composition of any one of claims 133-142, wherein the RNA encoding one or more antigenic polypeptides associated with a SARS-CoV-2 ancestral strain encodes an S protein associated with a Wuhan strain and comprising an amino acid sequence that is at least 85% identical to SEQ ID NO: 7.

145. The composition of claim 144, wherein the RNA encoding SEQ ID NO: 7 comprises a nucleotide sequence that is at least 85% identical to SEQ ID NO: 9.

146. The composition of any one of claims 133-145, wherein the RNA encoding one or more antigenic polypeptides associated with a second SARS-CoV-2 variant encodes an S protein associated with a BA.4/5 variant, and comprises an amino acid sequence that is at least 85% identical to SEQ ID NO: 69.

147. The composition of claim 146, wherein the RNA encoding SEQ ID NO: 69 comprises a nucleotide sequence that is at least 85% identical to SEQ ID NO: 70.

148. The composition of any one of claims 61-147, wherein:
the one or more first RNAs comprise:
(a) an RNA comprising a nucleotide sequence that encodes a SARS-CoV-2 Spike (S) polypeptide from an Omicron variant of SARS-CoV-2 (e.g., a BA.1, BA.2, BA.4/5, or XBB.1.5 variant); or
(b) an RNA comprising a nucleotide sequence that encodes a SARS-CoV-2 Spike (S) polypeptide from a SARS-CoV-2 ancestral strain (Wuhan strain) and an RNA comprising a nucleotide sequence that encodes a SARS-CoV-2 Spike (S) polypeptide from an Omicron variant of SARS-CoV-2 (e.g., a BA.1, BA.2, BA.4/5, or XBB.1.5 variant); and
wherein the one or more second RNAs comprise: (i) an RNA comprising a nucleotide sequence that encodes an HA polypeptide from an influenza A/H1N1 virus, (ii) an RNA comprising a nucleotide sequence that encodes an HA polypeptide from an influenza A/H3N2 virus, (iii) an RNA comprising a nucleotide sequence that encodes an HA polypeptide from an influenza B/Victoria lineage virus, and (iv) an RNA comprising a nucleotide sequence that encodes an HA polypeptide from an influenza B/Yamagata virus.

149. The composition of claim 148, where the H1N1 virus is A/Wisconsin/588/2019.

150. The composition of claim 149, wherein the HA polypeptide associated with A/Wisconsin/588/2019 comprises a sequence that is at least 85% identical to SEQ ID NO: 90.

151. The composition of claim 149 or 150, wherein the RNA comprising a nucleotide sequence encoding an HA polypeptide associated with A/Wisconsin/588/2019 comprises a sequence that is at least 85% identical to SEQ ID NO: 92.

152. The composition of any one of claims 148-151, where the H3N2 virus is A/Cambodia/e0826360/2020.

153. The composition of claim 152, wherein the HA polypeptide associated with A/Cambodia/e0826360/2020 comprises a sequence that is at least 85% identical to SEQ ID NO: 95.

154. The composition of claim 152 or 153, wherein the first RNA comprising a sequence encoding an HA polypeptide associated with A/Cambodia/e0826360/2020 comprises a sequence that is at least 85% identical to SEQ ID NO: 97.

155. The composition of any one of claims 148-154, where the B/Victoria lineage influenza virus is B/Washington/02/2019.

156. The composition of claim 155, wherein the HA polypeptide associated with B/Washington/02/2019 comprises a sequence that is at least 85% identical to SEQ ID NO: 100.

157. The composition of claim 155 or 156, wherein the RNA comprising a nucleotide sequence encoding an HA polypeptide associated with B/Washington/02/2019 comprises a sequence that is at least 85% identical to SEQ ID NO: 102.

158. The composition of any one of claims 148-157, where the B/Yamagata lineage influenza virus is B/Phuket/3073/2013.

159. The composition of claim 158, wherein the HA polypeptide associated with B/Phuket/3073/2013 comprises a sequence that is at least 85% identical to SEQ ID NO: 105.

160. The composition of claim 158 or 159, wherein the RNA comprising a sequence encoding an HA polypeptide associated with B/Phuket/3073/2013 comprises a nucleotide sequence that is at least 85% identical to SEQ ID NO: 107.

161. The composition of any one of claims 148-160, wherein the S polypeptide associated with a Wuhan strain comprises a sequence that is at least 85% identical to SEQ ID NO: 7.

162. The composition of any one of claims 148-161, wherein the RNA comprising a nucleotide sequence encoding an S polypeptide associated with a Wuhan strain comprises a sequence that is at least 85% identical to SEQ ID NO: 70.

163. The composition of any one of claims 148-162, wherein the Omicron variant is a BA.4/5 variant.

164. The composition of claim 163, wherein the S polypeptide associated with the BA.4/5 Omicron variant comprises a sequence that is at least 85% identical to SEQ ID NO: 69.

165. The composition of claim 163 or 164, wherein the RNA comprising a sequence encoding an S polypeptide associated with a BA.4/5 Omicron variant comprises a sequence that is at least 85% identical to SEQ ID NO: 70.

166. The composition of any one of claims 148-160, wherein the Omicron variant is an XBB.1.5 variant.

167. The composition of claim 166, wherein the S polypeptide associated with the XBB.1.5 Omicron variant comprises a sequence that is at least 85% identical to SEQ ID NO: 129.

168. The composition of claim 166 or 167, wherein the RNA comprising a sequence encoding an S polypeptide associated with an XBB.1.5 Omicron variant comprises a sequence that is at least 85% identical to SEQ ID NO: 130.

169. The composition of any one of claims 47-60 and 62-77, wherein at least one of the non-coding elements is or comprises:
(i) a 5′-UTR sequence that is or comprises a modified human alpha-globin 5′-UTR;
(ii) a 3′-UTR sequence that is or comprises a first sequence from the amino terminal enhancer of split (AES) messenger RNA and a second sequence from the mitochondrial encoded 12S ribosomal RNA;
(iii) a polyA sequence comprising at least 100 A nucleotides, wherein the first RNA and the second RNA each do not comprise a stretch of at least 30 contiguous C nucleotides between the 3′ UTR and the polyA sequence;
(iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; or
(v) a 5′ cap comprising a Cap1 structure and a cap proximal sequence comprising positions +1, +2, +3, +4, and +5 of the RNA; wherein:
(a) the Cap1 structure comprises m7(3′OMeG)(5′)ppp(5′)(2′OMeA₁)pG₂, wherein A₁ is position +1 of the RNA, and G₂ is position +2 of the RNA; and
(b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

170. The composition of any one of claims 47-169, wherein at least one of the same non-coding elements is or comprises:
(i) a 5′-UTR sequence that is or comprises a modified human alpha-globin 5′-UTR;
(ii) a 3′-UTR sequence that is or comprises a first sequence from the amino terminal enhancer of split (AES) messenger RNA and a second sequence from the mitochondrial encoded 12S ribosomal RNA;
(iii) a polyA sequence comprising at least 100 A nucleotides, wherein the first RNA and the second RNA each do not comprise a stretch of at least 30 contiguous C nucleotides between the 3′ UTR and the polyA sequence;
(iv) a polyA sequence comprising an interrupted sequence of A nucleotides, optionally wherein the interrupted sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence; and
(v) a 5′ cap comprising a Cap1 structure and a cap proximal sequence comprising positions +1, +2, +3, +4, and +5 of the RNA; wherein:
(a) the Cap1 structure comprises m7(3′OMeG)(5′)ppp(5′)(2′OMeA₁)pG₂, wherein A₁ is position +1 of the RNA, and G₂ is position +2 of the RNA; and
(b) the cap proximal sequence comprises A₁ and G₂ of the Cap1 structure, and a sequence comprising: A₃N₄N₅ at positions +3, +4 and +5 respectively of the RNA, wherein N₄ and N₅ are each independently selected from A, G, C, and U.

171. The composition of any one of claims 1-46, wherein each RNA comprises:
(i) a 5′ cap, wherein the 5 cap optionally comprises a cap1 structure;
(ii) a cap proximal sequence;
(iii) a 5′ UTR sequence, wherein the 5′ UTR is optionally a modified human alpha-globin 5-UTR;
(iv) a 3′ UTR sequence, wherein the 3′ UTR sequence optionally comprises a first sequence from the amino terminal enhancer of split (AES) messenger RNA and a second sequence from the mitochondrial encoded 12S ribosomal RNA; and/or
(v) a polyA sequence, wherein the polyA sequence optionally comprises 30 adenine nucleotides (SEQ ID NO: 174) followed by 70 adenine nucleotides (SEQ ID NO: 175), wherein the 30 adenine nucleotides (SEQ ID NO: 174) and the 70 adenine nucleotides (SEQ ID NO: 175) are separated by a linker sequence.

172. The composition of any one of claims 47-171, wherein the 5′ cap, cap proximal sequence, 5′ UTR sequence, 3′ UTR sequence, and polyA sequence are in 5′ to 3′ order.

173. The composition any one of claims 1-172, wherein each RNA comprises a 5′-cap that is or comprises m₂^{7,3′-O}Gppp(m₁^{2′-O})ApG.

174. The composition of any one of claims 47-169 and 171-173, wherein the 5′ UTR comprises or consists of a human alpha-globin 5′-UTR.

175. The composition of claim 174, wherein the human alpha-globin 5′-UTR comprises SEQ ID NO: 12.

176. The composition of any one of claims 47-169, and 171-175, wherein the 3′ UTR comprises or consists of a first sequence from the amino terminal enhancer of split (AES) messenger RNA and a second sequence from the mitochondrial encoded 12S ribosomal RNA

177. The composition of claim 176, wherein the 3′ UTR comprises or consists of a sequence according to SEQ ID NO: 13.

178. The composition of any one of claims 47-177, wherein the polyA tail sequence is an interrupted polyA tail sequence.

179. The composition of claim 178, wherein the interrupted polyA tail sequence comprises 30 adenine nucleotides (SEQ ID NO: 174) followed by 70 adenine nucleotides (SEQ ID NO: 175), wherein the 30 adenine nucleotides (SEQ ID NO: 174) and 70 adenine nucleotides (SEQ ID NO: 175) are separated by a linker sequence.

180. The composition of claim 179, wherein the interrupted polyA tail sequence comprises or consists of a ribonucleic acid sequence according to SEQ ID NO: 14.

181. The composition of any one of claims 47-180, wherein the sequence at the 3′ end of the 3′UTR (e.g., the sequence immediately adjacent to a sequence encoding an antigenic polypeptide) is CUCGAG or GGAUCCGAU.

182. The composition of any one of claims 1-181, wherein each RNA in the composition includes modified uridines in place of all uridines.

183. The composition of claim 182, wherein the modified uridines are each N1-methyl-pseudouridine.

184. The composition of any one of claims 47 to 183, wherein the first RNAs and second RNAs are present in a mass ratio of 1:5 to 5:1, 1:4 to 4:1, 1:3 to 3:1, or 1:2 to 2:1.

185. The composition of any one of claims 1 to 184, wherein each of the RNAs in the composition is formulated in nanoparticles.

186. The composition of any one of claims 47 to 185, wherein all of the first RNAs are co-formulated together in the same population of nanoparticles and all of the second RNAs are co-formulated together in the same population of nanoparticles, and wherein the first RNAs and the second RNAs are formulated in separate populations of nanoparticles.

187. The composition of any one of claims 47 to 186, wherein the first RNAs and the second RNAs are all co-formulated together in the same population of nanoparticles.

188. The composition of any one claims 1-60 and 96-186, wherein:
(a) each RNA encoding an antigenic polypeptide of an influenza A virus is coformulated in a first population of nanoparticles, and each RNA encoding an antigenic polypeptide of an influenza B virus is coformulated in a second population of nanoparticles;
(b) each RNA encoding an antigenic polypeptide of an influenza virus is formulated in a separate population of nanoparticles; or
(c) each RNA encoding an antigenic polypeptide of an influenza A virus is coformulated in a first population of nanoparticles, and each RNA encoding an antigenic polypeptide of an influenza B virus is formulated in separate nanoparticles.

189. The composition of any one of claims 185-188, wherein the nanoparticles comprise lipid nanoparticles, polyplexes (PLX), lipidated polyplexes (LPLX), liposomes, or polysaccharide nanoparticles.

190. The composition of claim 189, wherein the nanoparticles comprise lipid nanoparticles.

191. The composition of claim 190, wherein the lipid nanoparticles each comprise: a cationically ionizable lipid; and one or more neutral lipids, and a polymer-conjugated lipid.

192. The composition of claim 191, wherein the polymer-conjugated lipid comprises a PEG-conjugated lipid.

193. The composition of any one of claims 186-192, wherein the nanoparticles have an average diameter of about 50-150 nm.

194. The composition of any one of claims 1-193, further comprising:
(a) one or more third RNAs, each comprising a nucleotide sequence that encodes one or more antigenic polypeptides associated with a third infectious agent that is different from the first infectious agent and the second infectious agent; or
(b) one or more polypeptides of a third infectious agent.

195. The composition of claim 194, wherein the third infectious agent is a respiratory virus (e.g., a respiratory virus that is not a SARS-CoV-2 virus or an influenza virus).

196. The composition of claim 195, wherein the third infectious agent is a respiratory syncytial virus (RSV).

197. The composition of claim 196, wherein:
(i) the composition comprises one or more RNAs, each encoding an RSV polypeptide; or
(ii) the composition comprises one or more RSV polypeptides.

198. The composition of claim 197, wherein:
(i) the composition comprises one or more RNAS, each encoding an RSV F protein, a variant thereof, or an immunogenic fragment of an RSV F protein or a variant thereof; or
(ii) the composition comprises one or more RSV F proteins, an immunogenic variant thereof, or an immunogenic fragment of an RSV F protein or a variant thereof.

199. The composition of claim 197 or 198, wherein:
(i) the composition comprises one or more RNAs, each encoding a polypeptide of an RSV subtype A virus (e.g., an F protein of an RSV subtype A virus, a variant thereof, or an immunogenic fragment of an F protein of an RSV subtype B virus or a variant thereof), and one or more RNAs, each encoding a polypeptide of an RSV subtype B virus (e.g., an F protein of an RSV subtype B virus, a variant thereof, or an immunogenic fragment of an F protein of an RSV subtype B virus or a variant thereof); or
(ii) the composition comprises one or more polypeptides of an RSV subtype A virus (e.g., an F protein, a variant thereof, or an immunogenic fragment of an F protein or a variant thereof) and one or more polypeptides of an RSV subtype B virus (e.g., an F protein, a variant thereof, or an immunogenic fragment of an F protein or a variant thereof).

200. The composition of claim 198 or 199, wherein the RSV F protein, the variant, or the immunogenic fragment is stabilized in a prefusion confirmation.

201. The composition of any one of 194-200, comprising Arexvy™ or Abrysvo™.

202. A pharmaceutical composition comprising the composition of any one of claims 1-201 and at least one pharmaceutically acceptable excipient.

203. The pharmaceutical composition of claim 202, comprising a cryoprotectant, optionally wherein the cryoprotectant is or comprises sucrose.

204. The pharmaceutical composition of claim 202 or 203, wherein the pharmaceutical comprises an aqueous buffered solution, optionally wherein the aqueous buffered solution comprises one or more of Tris base, Tris HCl, NaCl, KCl, Na₂HPO₄, and KH₂PO₄.

205. The pharmaceutical composition of any one of claims 202-204, formulated to provide a dose of 100 μg or less of total RNA.

206. The pharmaceutical composition of claim 205, formulated to provide a dose of 90 μg of total RNA.

207. The pharmaceutical composition of claim 205, formulated to provide a dose of 60 μg of total RNA.

208. The pharmaceutical composition of claim 206, formulated to provide a dose of 30 μg of one or more first RNAs and a dose of 60 μg of one or more second RNAs.

209. The pharmaceutical composition of claim 206, formulated to provide a dose of 60 μg of one or more first RNAs and a dose of 30 μg of one or more second RNAs.

210. The pharmaceutical composition of claim 207, formulated to provide a dose of 30 μg of one or more first RNAs and a dose of 30 μg of one or more second RNAS.

211. The pharmaceutical composition of claim 206 or 208, comprising four second RNAs, each comprising a nucleotide sequence that encodes an antigenic polypeptide associated with a different influenza virus, and wherein the pharmaceutical composition is formulated to provide a dose of 15 μg of each second RNA.

212. The pharmaceutical composition of claim 207 or 209, comprising four second RNAs, each comprising a nucleotide sequence that encodes an antigenic polypeptide associated with a different influenza virus, and wherein the pharmaceutical composition is formulated to provide a dose of 7.5 μg of each second RNA.

213. The pharmaceutical composition of any one of claims 206-208 and 210-212, comprising two first RNAs, each comprising a nucleotide sequence that encodes an antigenic polypeptide associated with a different coronavirus virus, and wherein the pharmaceutical composition is formulated to provide a dose of 15 μg of each first RNA.

214. The pharmaceutical composition of claim 209, comprising two first RNAs, each comprising a nucleotide sequence that encodes an antigenic polypeptide associated with a different coronavirus virus, and wherein the pharmaceutical composition is formulated to provide a dose of 30 μg of each first RNA.

215. A method comprising administering to a subject one or more doses of the composition of any one of claims 1-201 or one or more doses of the pharmaceutical composition of any one of claims 202-214.

216. The method of claim 215, wherein the method is a method of treating a coronavirus disease and influenza disease.

217. The method of claim 215, wherein the method is a method of (i) preventing a coronavirus disease and an influenza disease or (ii) inducing an immune response against a coronavirus and/or an influenza virus.

218. The method of any one of claims 215-217, wherein each the one or more doses of the composition or each of the one or more doses of the pharmaceutical composition is co-administered with a vaccine against a third infectious agent.

219. The method of claim 218, wherein the third infectious agent is a virus that can cause a respiratory disease.

220. The method of claim 219, wherein the third infectious agent is RSV.

221. The method of claim 220, wherein the vaccine against the third infectious agent is Arexvy™ or Abrysvo™.

222. The method of any one of claims 218-221, wherein:
the vaccine against the third infectious agent is mixed with the one or more doses of the composition or the one or more doses of the pharmaceutical compositions immediately before administering to the subject; or
the vaccine against the third infectious agent is administered separately from the one or more doses of the composition or the one or more doses of the pharmaceutical compositions (e.g., wherein the vaccine against the third infectious agent and the one or more doses of the composition or the one or more doses of the pharmaceutical composition are administered to the subject at separate injection sites (e.g., on opposite arms)).

223. The composition of any one of claims 1-201 or the pharmaceutical composition of any one of claims 202-214, for use in the treatment of a coronavirus disease and an influenza disease, wherein the use comprises administering one or more doses of the composition or pharmaceutical composition to a subject.

224. The composition of any one of claims 1-201 or the pharmaceutical composition of any one of claims 202-214, for use in:
(a) the prevention of a coronavirus disease and an influenza disease, or
(b) inducing an immune response against a coronavirus and an influenza virus,
wherein the use comprises administering one or more doses of the composition or pharmaceutical composition to a subject.

225. The method of any one of claims 215-222, or the composition or pharmaceutical composition for use of claim 223 or 224, wherein the method or the use comprises administering two or more doses of the composition or pharmaceutical composition to the subject.

226. The method or composition or pharmaceutical composition for use of claim 225, wherein the two doses are administered at least about 21 days apart.

227. The method of any one of claims 215-222, or the composition or pharmaceutical composition for use of claim 225 or 226, wherein the method or the use comprises administering three or more doses of the composition or pharmaceutical composition to the subject.

228. The method of any one of claims 215-222, or the composition or pharmaceutical composition for use of claim 223 or 224, wherein the subject has previously been exposed to a coronavirus and/or an influenza virus (e.g., by vaccination and/or by infection).

229. The method or composition or pharmaceutical composition for use of any one of claims 215-228, wherein the method or use induces an immune response in the subject against one or coronaviruses and one or more influenza viruses.

230. The method or composition or pharmaceutical composition for use of claim 229, wherein the immune response comprises a B-cell response.

231. The method or composition or pharmaceutical composition for use of claim 230, wherein the B cell response comprises production of antibodies directed against the one or more antigens.

232. The method or composition or pharmaceutical composition for use of claim 230 or 231, wherein the immune response comprises a T cell response.

233. The method or composition or pharmaceutical composition for use of claim 232, wherein the T-cell response is or comprises a CD4+ T cell response.

234. The method or composition or pharmaceutical composition for use of claim 232 or 233, wherein the T-cell response is or comprises a CD8+ T cell response.

235. Use of the composition of any one of claims 1-201, or the pharmaceutical composition of any one of claims 202-214 in the treatment of a coronavirus disease and an influenza disease in a subject.

236. Use of the composition of any one of claims 1-201 or the pharmaceutical composition of any one of claims 202-215 in the prevention of a coronavirus disease and an influenza disease in a subject.

237. Use of the composition of any one of claims 1-201 or the pharmaceutical composition of any one of claims 202-215 in inducing an immune response in a subject against one or more coronaviruses and one or more influenza viruses.

238. A method for inducing an immune response against a first infectious agent and a second infectious agent, wherein the method comprises administering
(i) a first nanoparticle (e.g., LNP) formulated RNA comprising a nucleotide sequence encoding an antigenic polypeptide associated with a first infectious agent; and
(ii) a second nanoparticle (e.g., LNP) formulated RNA comprising a nucleotide sequence encoding an antigenic polypeptide associated with a second infectious agent,
wherein the immune response induced against each of the first and the second infectious agents is greater than the immune response induced when the nanoparticles are administered separately.

239. A method for reducing the amount of a first nanoparticle (e.g. LNP) formulated RNA required to produce an immune response against a first infectious agent, wherein the RNA of the first nanoparticle-formulated RNA comprises a nucleotide sequence encoding one or more antigenic polypeptides associated with a first infectious agent,
wherein the method comprises co-administering a second nanoparticle (e.g., LNP)-formulated RNA comprising a nucleotide sequence encoding one or more antigenic polypeptides associated with a second infectious agent, and
wherein the first infectious agent differs from the second infectious agent.

240. A composition comprising:
one or more RNAs, each encoding a polypeptide of a first infectious agent; and
one or more polypeptides of a second infectious agent.

241. The composition of claim 240, wherein the first infectious agent is a coronavirus.

242. The composition of claim 241, wherein the coronavirus is a SARS-CoV-2 virus.

243. The composition of claim 242, wherein the one or more RNAs each encode a SARS-CoV-2 S protein, a variant thereof, or an immunogenic fragment of a SARS-CoV-2 S protein or variant thereof.

244. The composition of claim 243, comprising one or more RNAs, each encoding a SARS-CoV-2 S protein, a variant thereof, or an immunogenic fragment of a SARS-CoV-2 S protein or variant thereof of a Wuhan strain or a SARS-CoV-2 variant (e.g., an Omicron variant (e.g., an Omicron BA.1, BA.2, BA.4/5, or an XBB.1.5 variant (e.g., an RNA described herein))).

245. The composition of any one of claims 240-244, wherein the second infectious agent is an influenza virus.

246. The composition of claim 245, wherein the composition comprises one or more polypeptides of one or more influenza viruses (e.g., one or more polypeptides of two or more influenza virus strains (e.g., one or more polypeptides of four or more influenza virus strains that are prevalent or which have been predicted to be prevalent in a relevant jurisdiction)).

247. The composition of claim 245 or 246, comprising a commercially available influenza virus (e.g., a recombinant commercially available influenza virus described herein).

248. The composition of claim 247, wherein the commercially available influenza virus is Flublok.

249. The composition of any one of claims 239-244, wherein the second infectious agent is an RSV.

250. The composition of claim 249, comprising one or more polypeptides associated with a first RSV subtype and one or more polypeptides of a second RSV subtype.

251. The composition of claim 249 or 250, wherein the polypeptide of the second infectious agent is an RSV F protein, a variant thereof, or an immunogenic fragment of either of an RSV F protein or a variant thereof.

252. The composition of claim 251, wherein the RSV F protein, the variant thereof, or the immunogenic fragment thereof, comprises one or more mutations that stabilize a prefusion confirmation of the F protein.

253. The composition of claim 252, comprising Arexvy™ or ABRYSVO™.

254. The composition of any one of claims 239-253, further comprising one or more polypeptides of a third infectious agent.

255. The composition of claim 254, comprising:
one or more RNAs, each encoding one or more polypeptides of a coronavirus (e.g., a SARS-CoV-2 S protein, a variant thereof, or an immunogenic fragment of either of the foregoing);
one or more polypeptides of one or more influenza viruses; and
one or more polypeptides of one or more RSVs.

256. The composition of claim 255, comprising:
an RNA encoding a SARS-CoV-2 S protein of an Omicron variant (e.g., an RNA encoding an S protein of an Omicron BA.1, BA.4/5, or XBB.1.5 variant described herein);
a recombinant influenza vaccine (e.g., as described herein (e.g., a FluBlok vaccine)); and
an RSV vaccine comprising a prefusion-stabilized F protein, or a variant or immunogenic fragment thereof (e.g., an RSV vaccine described herein (e.g., Arexvy™ or ABRYSVO™)).

257. A combination comprising a SARS-CoV-2 vaccine comprising one or more mRNAs encoding a prefusion stabilized SARS-CoV-2 spike protein or a variant thereof; and
(a) an influenza vaccine comprising (i) one or more mRNAs encoding an HA protein of an influenza virus, or (ii) one or HA polypeptides, and/or
(b) an RSV vaccine comprising one or more prefusion stabilized RSV F proteins, or a variant or immunogenic fragment thereof.

258. The combination of claim 257, wherein the one or more mRNAs encoding a prefusion stabilized SARS-CoV-2 spike protein or a variant thereof is formulated as an LNP.

259. The combination of claim 257 or 258, wherein the one or more mRNAs encoding an HA protein of an influenza virus is formulated as an LNP.

260. The combination of any one of claims 257-259, wherein the (1) SARS-CoV-2 vaccine and the (2) influenza vaccine or RSV vaccine are provided in separate containers (e.g., vials or syringes).

261. The combination of any one of claims 257-259, wherein the (1) SARS-CoV-2 vaccine and the (2) influenza vaccine or RSV vaccine are provided in a single containers (e.g., vial or syringe).

262. The combination of any one of claims 257-261, comprising the SARS-CoV-2 vaccine, the influenza vaccine, and the RSV vaccine.

263. The combination of claim 262, wherein the SARS-CoV-2 vaccine, the influenza vaccine, and the RSV vaccine are provided in a single container (e.g., a vial or syringe).

264. The combination of claim 262, wherein the SARS-CoV-2 vaccine, the influenza vaccine, and the RSV vaccine are provided in separate containers (e.g., separate vials and/or syringes).

265. The combination of claim 262, wherein:
(a) the SARS-CoV-2 vaccine and the influenza vaccine are provided in a single container, and the RSV vaccine is provided in a separate container; or
(b) the SARS-CoV-2 vaccine and RSV vaccine are provided in a single container, and the influenza vaccine is provided in a separate container.

266. The combination any one of claims 257-265, wherein the SARS-CoV-2 vaccine is BNT162b2 (e.g., a monovalent or bivalent vaccine described herein).

267. The combination any one of claims 257-266, wherein the influenza vaccine is a recombinant influenza vaccine (e.g., as described herein (e.g., a FluBlok vaccine)); or comprises an inactivated influenza virus (e.g., Fluzone).

268. The combination of any one of claims 257-267, wherein the RSV vaccine comprises a prefusion-stabilized F protein or a variant or immunogenic fragment thereof (e.g., an RSV vaccine described herein (e.g., Arexvy™ or ABRYSVO™)).
